(19) United States
(12) Patent Application Publication
(10) Pub. No.: US 2025/0197387 A1
(43) Pub. Date: Jun. 19, 2025

(54) BCL-XL DEGRADERS AND USES THEREOF

(71) Applicant: Kymera Therapeutics, Inc., Watertown, MA (US)

(72) Inventors: Robert AVERSA, Watertown, MA (US); Bin YANG, Watertown, MA (US); Huijun DONG, Watertown, MA (US)

(21) Appl. No.: 18/692,584

(22) PCT Filed: Sep. 16, 2022
(86) PCT No.: PCT/US2022/043865

§ 371 Date: Mar. 15, 2024

Related U.S. Application Data
(60) Provisional application No. 63/245,637, filed on Sep. 17, 2021.
(60) Provisional application No. 63/350,208, filed on Jun. 8, 2022.

(51) Int. Cl. C07D 417/12; A61K 31/427; A61K 31/4725; A61P 35/00; C07D 417/14

(52) U.S. Cl. CPC C07D 417/12; A61K 31/427; A61K 31/4725; A61P 35/00; C07D 417/14

(57) ABSTRACT

The present application relates novel bifunctional compounds, which function to recruit BCL-XL protein to E3 ubiquitin ligase for degradation, and methods of preparation and uses thereof. In particular, the present disclosure provides bifunctional compounds, which find utility as modulators of targeted ubiquitination of BCL-XL protein, which are then degraded and/or otherwise inhibited by the bifunctional compounds as described herein. Also provided are monovalent compounds, which find utility as inducers of targeted ubiquitination of BCL-XL protein, which are then degraded and/or otherwise inhibited by the monovalent compounds as described herein.

### CROSS-REFERENCE TO RELATED APPLICATIONS

This application claims the benefit of priority to U.S. Provisional Appl. No. 63/245,637, filed Sep. 17, 2021, and U.S. Provisional Appl. No. 63/350,208, filed Jun. 8, 2022, the entirety of each of which is herein incorporated by reference.

### TECHNICAL FIELD OF THE INVENTION

The present invention relates to compounds and methods useful for the modulation of B-cell lymphoma-extra large (BCL-XL) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”: Berndsen et al. (Nat. Struct. Mol. Biol., 2014, 21, 301-307) titled “New insights into ubiquitin E3 ligase mechanism”: Deshaies et al. (Ann. Rev. Biochem., 2009, 78, 399-434) titled “RING domain E3 ubiquitin ligases.”: Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy; in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups; (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17 (6): 551-555: Schnnekloth JS Jr., Chembiochem, 2005, 6 (1): 40-46).

The BCL-2 (B-cell lymphoma-2) family of proteins is a group of regulator proteins that plays a central role in regulating cell death by either inducing (pro-apoptotic) or inhibiting (anti-apoptotic) apoptosis. The anti-apoptotic BCL-2 family of proteins, such as BCL-2, BCL-XL, BCL-W, and MCL-1, are attractive target for the development of novel anti-cancer agents.

There is an ongoing need in the art for effective treatments for disease, especially cancer. As such, small molecule therapeutic agents that leverage E3 ligase mediated protein degradation to cancer associated proteins such as B-cell lymphoma-extra large (BCL-XL) hold promise as therapeutic agents. Accordingly, there remains a need to find compounds that are BCL-XL degraders useful as therapeutic agents.

### SUMMARY OF THE INVENTION

The present application relates novel bifunctional compounds, which function to recruit BCL-XL protein to E3 ubiquitin ligase for degradation, and methods of preparation and uses thereof. In particular, the present disclosure provides bifunctional compounds, which find utility as modulators of targeted ubiquitination of BCL-XL protein, which are then degraded and/or otherwise inhibited by the bifunctional compounds as described herein. Also provided are monovalent compounds, which find utility as inducers of targeted ubiquitination of BCL-XL protein, which are then degraded and/or otherwise inhibited by the monovalent compounds as described herein. An advantage of the compounds provided herein is that a broad range of pharmacological activities is possible, consistent with the degradation/inhibition of BCL-XL protein. In addition, the description provides methods of using an effective amount of the compounds as described herein for the treatment or amelioration of a disease condition, such as cancer.

The present application further relates to targeted degradation of BCL-XL protein through the use of bifunctional molecules, including bifunctional molecules that link a cereblon-binding moiety to a ligand that binds BCL-XL protein.

It has now been found that compounds of this invention, and pharmaceutically acceptable compositions thereof, are effective as degraders of BCL-XL protein. Such compounds have the general formula I:

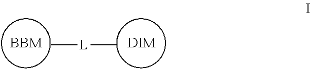

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating BCL-XL protein. Such diseases, disorders, or conditions include those described herein.

Compounds provided by this invention are also useful for the study of BCL-XL protein in biological and pathological phenomena; the study of intracellular signal transduction pathways occurring in bodily tissues; and the comparative evaluation of new BCL-XL inhibitors or BCL-XL degraders or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention:

Compounds of the present invention, and compositions thereof, are useful as degraders and/or inhibitors of BCL-XL protein.

In certain embodiments, the present invention provides a compound of formula I:

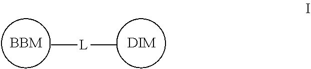

or a pharmaceutically acceptable salt thereof, wherein:
BBM is a BCL-XL binding moiety capable of binding to BCL-XL;
L is a bivalent moiety that connects BBM to DIM; and
DIM is a degradation inducing moiety selected from an E3 ubiquitin ligase binding moeity (LBM), lysine mimetic, and hydrogen.

### 2. Compounds and Definitions:

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Without limitation, a bridged bicyclic group may contain two or more bridges, e.g., adamantanyl. Exemplary bridged bicyclics include:

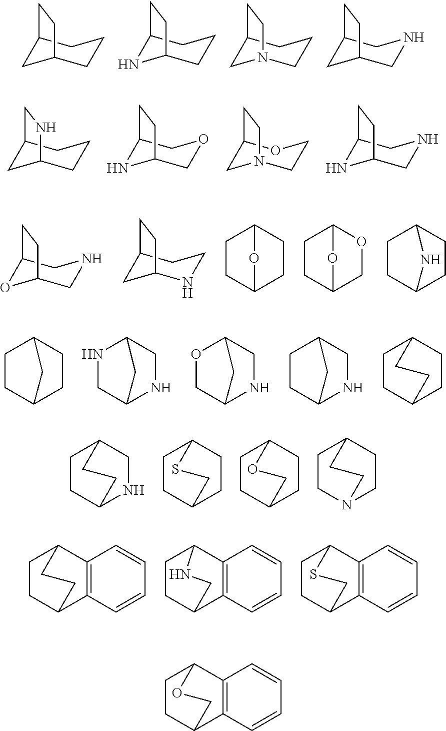

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

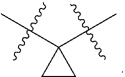 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms: having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring.” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or ⁺NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle.” “heterocyclyl.” “heterocyclyl ring.” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable.” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen: —(CH₂)₀₋₄R^{o}; —(CH₂)₀₋₄OR^{o}; —O(CH₂)₀₋₄R^{o}; —O—(CH₂)₀₋₄C(O)OR^{o}; —(CH₂)₀₋₄CH(OR^{o}₂; —(CH₂)₀₋₄SR^{o}; —(CH₂)⁰⁻⁴Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN: —N₃; —(CH₂)₀₋₄N(R^{o}₂)₂; (CH₂)₀₋₄N(R^{o}C(O)R^{o}; —N(R^{o}C(S)R^{o}; —(CH₂)₀₋₄N(R^{o}C(O)NR^{o}₂; —N(R^{o}C(S)NR^{o}₂; —(CH₂)₀₋₄N(R^{o}C(O)OR^{o}; —N(R^{o}N(R^{o}C(O)R^{o}); —N(R^{o}N(R^{o}C(O)NR^{o}₂); —N(R^{o}N(R^{o}C(O)OR^{o}; —(CH₂)₀₋₄C(O)R^{o}; —C(S) R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o}S(O)₂NR^{o}₂; —N(R^{o}S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene) C(O)O—N(R^{o})₂, wherein each R^{o} may be substituted as defined below and is independently hydrogen. C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂—; S—, wherein each independent occurrence of R* is selected from hydrogen. C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{o}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include-R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S) NR^{†}₂, —C(NH)NR^{†}₂, or —N(R^{†})S(O)²R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example S. M. Berge et al., describe pharmaceutically acceptable salts in detail in J. Pharmaceutical Sciences. 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide. 2-hydroxy-ethanesulfonate, lactobionate, lactate, laurate, lauryl sulfate, malate, maleate, malonate, methanesulfonate. 2-naphthalenesulfonate, nicotinate, nitrate, oleate, oxalate, palmitate, pamoate, pectinate, persulfate. 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or case of purification, e.g., using an acidic or basic mobile phase during chromatography. Salts forms of the provided compounds formed during chromotagraphic purification are comtemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure: for example, the R and S configurations for each asymmetric center. Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

The term “prodrug” refers to a compound that is made more active in vivo. The present compounds can also exist as prodrugs, as described in Hydrolysis in Drug and Prodrug Metabolism: Chemistry Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug.” refers to those prodrugs or zwitterions which are suitable for use in contact with the tissues of patients without undue toxicity, irritation, and allergic response, are commensurate with a reasonable benefit/risk ratio, and are effective for their intended use.

As used herein, the term “inhibitor” is defined as a compound that binds to and/or inhibits an BCL-XL protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both an BCL-XL protein and an E3 ligase with measurable affinity resulting in the ubiquitination and subsequent degradation of the BCL-XL protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium. ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”. “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G. BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a BCL-XL protein activity between a sample comprising a compound of the present invention, or composition thereof, and a BCL-XL protein, and an equivalent sample comprising a BCL-XL protein, in the absence of said compound, or composition thereof.

### 3. Description of Exemplary Embodiments:

As described above, in certain embodiments, the present invention provides a compound of formula I:

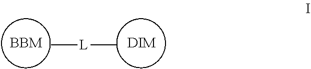

or a pharmaceutically acceptable salt thereof, wherein:
BBM is a BCL-XL protein binding moiety capable of binding to BCL-XL;
L is a bivalent moiety that connects BBM to DIM; and
DIM is a degradation inducing moiety selected from an E3 ubiquitin ligase binding moeity (LBM), lysine mimetic, and hydrogen.

### BCL-XL Binding Moiety (BBM)

As described and defined herein, BBM is a BCL-XL protein binding moiety. In some embodiments, BBM is a selective BCL-XL protein binding moiety. In some embodiments, BBM binds selectively to BCL-XL over other anti-apoptotic BCL-2 family proteins, such as BCL-2.

In some embodiments, BBM binds other anti-apoptotic BCL-2 family proteins, such as BCL-2. In some embodiments, BBM is a selective BCL-2 protein binding moiety. In some embodiments, BBM binds selectively to BCL-2 over other anti-apoptotic BCL-2 family proteins, such as BCL-XL.

Such binders are well known to one of ordinary skill in the art and including ABT-737 (US 20070072860), navitoclax (ABT-263, WO 2009155386), venetoclax (ABT-199, WO 2010138588), obatoclax (GX 15-070, WO 2004106328), pelcitoclax (APG-1252), (−)-gossypol (AT-101, WO 2002097053), sabutoclax (BI-97C1, WO 2010120943), TW-37 (WO 2006023778), BM-1252 (APG-1252), A-1155463 (WO 2010080503 and WO 2010080478), A-1293102, A-1331852 (WO 2013055897 and WO 2013055895), AZD4320 (WO 2012017251), WEHI-539 (Lessene, Guillaume, et al. “Structure-guided design of a selective BCL-XL inhibitor.” Nature Chem. Bio. 2013, 9 (6):390-397), and other binders disclosed by the University of Michighan (WO 2018027097), Genentech (WO 2008061208), Novartis (WO2011029842A1), and Servier (WO 2021018857 and WO 2021018858), the entirety of each or which is herein incorporated by reference.

As defined herein and described below, wherein a formula is depicted using square brackets, e.g.,

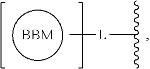

L is attached to a modifiable carbon, oxygen, or nitrogen atom within BBM including substitution or replacement of a defined group in BBM.

In certain embodiments, the present invention provides a compound of formula I, wherein BBM is a BCL-XL binding moiety thereby forming a compound of formula I-aa:

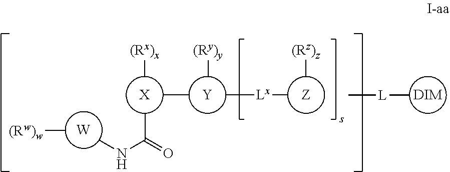

or a pharmaceutically acceptable salt, wherein L and DIM are as defined above and described in embodiments herein, and wherein;
Ring W and Ring Z are, independently, a ring selected from phenyl, naphthyl, a 5-10 membered heteroaryl containing 1-4 heteroatoms independently selected from nitrogen, oxygen, selenium, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring X is a bicyclic ring selected from a 9-11 membered partially unsaturated heterocyclylenyl with 1-4 heteroatoms independently selected from nitrogen, oxygen, and sulfur, and a 9-10 membered heteroarylenyl containing 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{w}, R^{x}, R^{y}, and R^{z}, are, independently, hydrogen, R^{A}, halogen, —CN, —NO₂, —OR, —SR, —N(R)₂, —Si(R)₂, —S(O) ¿R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of L^{x} are independently replaced by an optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s is 0 or 1; and
w, x, y, and z are, independently, 0, 1, 2, 3, or 4.

In certain embodiments, the present invention provides a compound of formula I, wherein BBM is a BCL-XL binding moiety thereby forming a compound of formula I-bb:

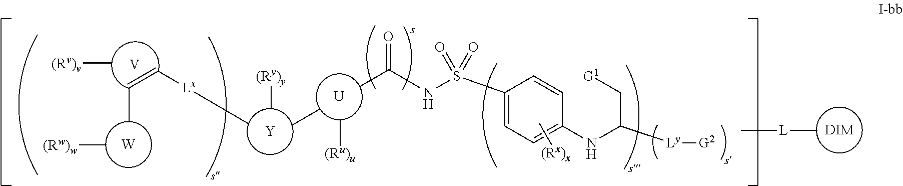

or a pharmaceutically acceptable salt, wherein L and DIM are as defined above and described in embodiments herein, and wherein;
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
Ring V is a bivalent ring selected from a 5-6 membered partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
R^{u}, R^{v}, R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R, S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G¹ is —S-aryl, —S-heteroaryl, or —R^{A};
G² is hydrogen or

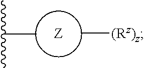

Ring Z is a ring selected from phenyl, naphthyl, a 5-10 membered heteroaryl containing 1-4 heteroatoms independently selected from nitrogen, oxygen, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur; L^{x} and L^{y} are, independently, a covalent bond or a bivalent, saturated or partially unsaturated, straight or branched C₁₋₅ hydrocarbon chain, wherein 0-3 methylene units of L^{x} or L^{y} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
S, s″, and s″ are, independently, 0 or 1;
s′ is 1 or 2; and
u, v, w, X, y, and z are, independently, 0, 1, 2, 3, or 4.

In certain embodiments, the present invention provides a compound of formula I, wherein BBM is a BCL-XL binding moiety thereby forming a compound of formula I-bb′:

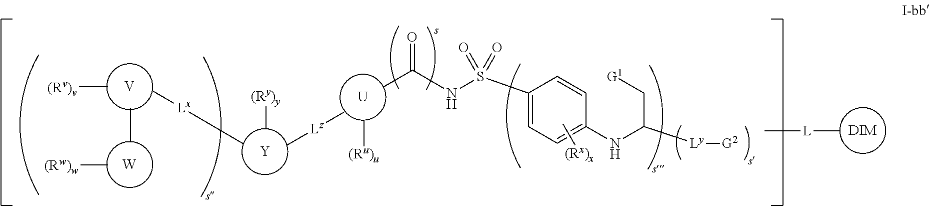

or a pharmaceutically acceptable salt, wherein L and DIM are as defined above and described in embodiments herein, and wherein;
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
R^{u}, R^{v}, R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R, S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G¹ is —S-aryl, —S-heteroaryl, or —R^{A};
G² is hydrogen or

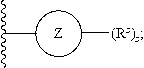

Ring Z is a ring selected from phenyl, naphthyl, a 5-10 membered heteroaryl containing 1-4 heteroatoms independently selected from nitrogen, oxygen, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{y}, and L^{z} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
S, s″, and s″ are, independently, 0 or 1;
s′ is 1 or 2; and
u, v, w, x, y, and z are, independently, 0, 1, 2, 3, or 4.

In certain embodiments, the present invention provides a compound of formula I, wherein BBM is a BCL-XL binding moiety thereby forming a compound of formula I-cc:

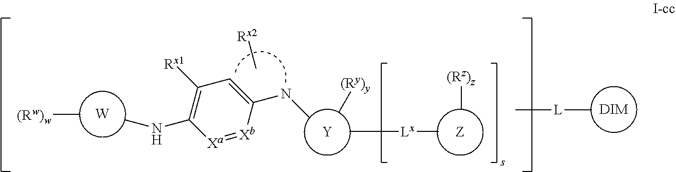

or a pharmaceutically acceptable salt, wherein L and DIM are as defined above and described in embodiments herein, and wherein:
Ring W and Ring Z are, independently, a ring selected from phenyl, naphthyl, a 5-10 membered heteroaryl containing 1-4 heteroatoms independently selected from nitrogen, oxygen, selenium, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;

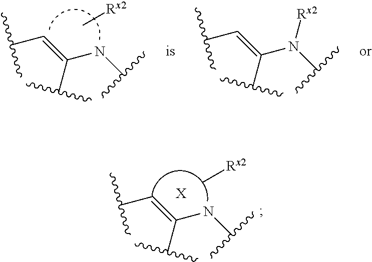

Ring X is a fused ring selected from a 5-6 membered saturated or partially unsaturated heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{w}, R^{x1}, R^{x2}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R, S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of L^{x} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
X^{a} and X^{b} are, independently, a carbon atom or a nitrogen atom;
s is 0 or 1; and
w, y, and z are, independently, 0, 1, 2, 3, or 4.

In certain embodiments, the present invention provides a compound of formula I, wherein BBM is a BCL-XL binding moiety thereby forming a compound of formula I-dd:

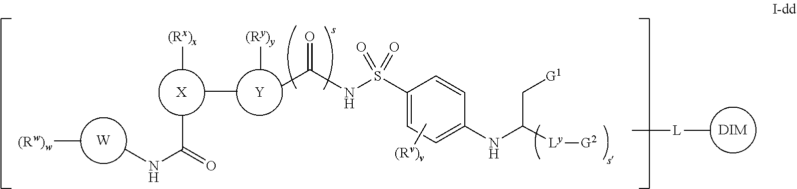

or a pharmaceutically acceptable salt, wherein L and DIM are as defined above and described in embodiments herein, and wherein:
Ring W is a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring X is a bicyclic ring selected from a 9-11 membered partially unsaturated heterocyclylenyl with 1-4 heteroatoms independently selected from nitrogen, oxygen, and sulfur, and a 9-10 membered heteroarylenyl containing 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{v}, R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O) ¿N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{y} is a covalent bond or a bivalent, saturated or partially unsaturated, straight or branched C₁₋₅ hydrocarbon chain, wherein 0-3 methylene units of L^{x} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
G¹ is —S-aryl, —S-heteroaryl, or —R^{A};
G² is hydrogen or

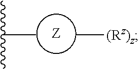

Ring Z is a ring selected from phenyl, naphthyl, a 5-10 membered heteroaryl containing 1-4 heteroatoms independently selected from nitrogen, oxygen, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
s is 0 or 1;
s′ is 1 or 2; and
v, w, x, y, and z are, independently, 0, 1, 2, 3, or 4.

As defined above and described herein, Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur.

In some embodiments, Ring U is phenylenyl. In some embodiments, Ring U is a 5-15 membered saturated or partially unsaturated heterocyclylenyl with 1-4 heteroatoms independently selected from nitrogen, oxygen, and sulfur. In some embodiments, Ring U is a 5-10 membered heteroarylenyl containing 1-4 heteroatoms independently selected from nitrogen, oxygen, and sulfur.

In some embodiments, Ring U is

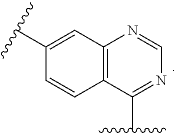 [SMILES: *c1ccc2c(*)ncnc2c1]

In some embodiments, Ring U is

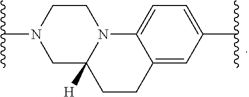 [SMILES: *c1ccc2c(c1)CC[C@@H]1CN(*)CCN21]

In some embodiments, Ring U is

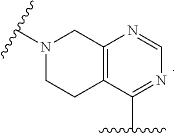 [SMILES: *c1ncnc2c1CCN(*)C2]

In some embodiments, Ring U is selected from those depicted in Table 1, below.

As defined above and described herein, Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur.

In some embodiments, Ring V is a 5-6 membered saturated or partially unsaturated carbocyclylenyl. In some embodiments, Ring V is a 5-6 membered saturated or partially unsaturated heterocyclylenyl with 1-4 heteroatoms independently selected from nitrogen, oxygen, and sulfur. In some embodiments, Ring V is a 5-6 membered heterocyclylenyl with 1-4 heteroatoms independently selected from nitrogen, oxygen, and sulfur. In some embodiments, Ring V is a 5-6 membered heteroarylenyl with 1-4 heteroatoms independently selected from nitrogen, oxygen, and sulfur.

In some embodiments, Ring V is cyclohexenyl. In some embodiments, Ring V is pyrrolylenyl.

In some embodiments, Ring V is selected from those depicted in Table 1, below.

As defined above and described herein, Ring W and Ring Z are, independently, a ring selected from phenyl, naphthyl, a 5-10 membered heteroaryl containing 1-4 heteroatoms independently selected from nitrogen, oxygen, selenium, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur.

In some embodiments, Ring W is phenyl. In some embodiments, Ring W is naphthyl. In some embodiments, Ring W is a 5-10 membered heteroaryl containing 1-4 heteroatoms independently selected from nitrogen, oxygen, selenium, and sulfur. In some embodiments, Ring W is a 3-11 membered saturated or partially unsaturated monocyclic, bicyclic, bridged bicylic, or spirocyclic carbocyclyl. In some embodiments, Ring W is a 3-11 membered saturated or partially unsaturated monocyclic, bicyclic, bridged bicylic, or spirocyclic heterocyclyl with 1-4 heteroatoms independently selected from nitrogen, oxygen, and sulfur.

In some embodiments, Ring W is thiazoyl. In some embodiments, Ring W is pyridyl. In some embodiments, Ring W is benzothiazolyl. In some embodiments, Ring W is thiazolopyridyl.

In some embodiments, Ring W is

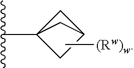

In some embodiments, Ring W is

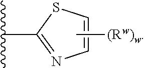

In some embodiments, Ring W is

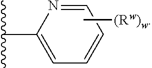

In some embodiments, Ring W is

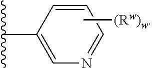

In some embodiments, Ring W is

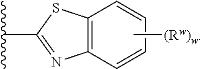

In some embodiments, Ring W is

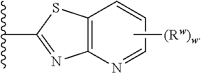

In some embodiments, Ring W is

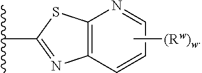

In some embodiments, Ring Z is phenyl. In some embodiments, Ring Z is naphthyl. In some embodiments, Ring Z is a 5-10 membered heteroaryl containing 1-4 heteroatoms independently selected from nitrogen, oxygen, selenium, and sulfur. In some embodiments, Ring Z is a 3-11 membered saturated or partially unsaturated monocyclic, bicyclic, bridged bicylic, or spirocyclic carbocyclyl. In some embodiments, Ring Z is a 3-11 membered saturated or partially unsaturated monocyclic, bicyclic, bridged bicylic, or spirocyclic heterocyclyl with 1-4 heteroatoms independently selected from nitrogen, oxygen, and sulfur.

In some embodiments, Ring Z is azetidinyl. In some embodiments, Ring Z is cyclopropyl. In some embodiments, Ring Z is cyclopentyl. In some embodiments, Ring Z is cyclohexyl. In some embodiments, Ring Z is piperzinyl. In some embodiments, Ring Z is morpholinyl. In some embodiments, Ring Z is pyridyl. In some embodiments, Ring Z is pyrazolyl. In some embodiments, Ring Z is

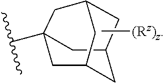

In some embodiments, Ring Z is

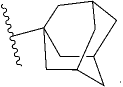 [SMILES: *C12CC3CC(CC(C3)C1)C2]

In some embodiments, Ring W and Ring Z are selected from those depicted in Table 1, below.

As defined above and described herein,

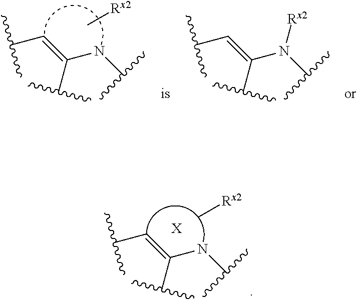

In some embodiments,

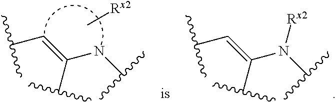

In some embodiments,

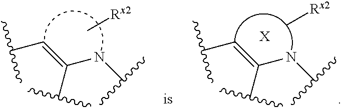

In some embodiments,

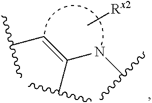

is selected from those depicted in Table 1, below.

As defined above and described herein, Ring X is a fused ring selected from a 5-6 membered saturated or partially unsaturated heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur.

In some embodiments, Ring X is a 5-6 membered saturated or partially unsaturated heterocyclylenyl with 1-4 heteroatoms independently selected from nitrogen, oxygen, and sulfur. In some embodiments, Ring X is a 5-6 membered heteroarylenyl containing 1-4 heteroatoms independently selected from nitrogen, oxygen, and sulfur. In some embodiments, Ring X is

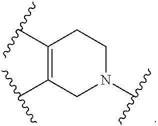 [SMILES: *C1=C(*)CN(*)CC1]

In some embodiments, Ring X is

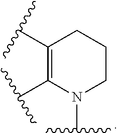 [SMILES: *C1=C(*)N(*)CCC1]

In some embodiments, Ring X is selected from those depicted in Table 1, below.

As defined above and described herein, Ring Y is a bivalent ring selected from phenylenyl, a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur.

In some embodiments, Ring Y is phenylenyl. In some embodiments, Ring Y is a 5-10 membered heteroarylenyl containing 1-4 heteroatoms independently selected from nitrogen, oxygen, and sulfur. In some embodiments, Ring Y is a 5-7 membered saturated or partially unsaturated carbocyclylenyl. In some embodiments, Ring Y is a 5-7 membered saturated or partially unsaturated heterocyclylenyl with 1-4 heteroatoms independently selected from nitrogen, oxygen, and sulfur. In some embodiments, Ring Y is a 5-6 membered heteroarylenyl containing 1-4 heteroatoms independently selected from nitrogen, oxygen, and sulfur.

In some embodiments, Ring Y is pyridylenyl. In some embodiments, Ring Y is thiazoylenyl. In some embodiments, Ring Y is piperzinylenyl.

In some embodiments, Ring Y is selected from those depicted in Table 1, below.

As defined above and described herein, G¹ is —S-aryl, —S-heteroaryl, or —R^{A}.

In some embodiments, G¹ is —S-aryl. In some embodiments, G¹ is —S-heteroaryl. In some embodiments, G¹ is —R^{A}.

In some embodiments, G¹ is —SPh.

In some embodiments, G¹ is selected from those depicted in Table 1, below.

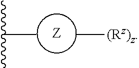

As defined above and described herein, G² is hydrogen or

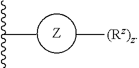

In some embodiments, G² is hydrogen. In some embodiments, G² is

In some embodiments, G² is selected from those depicted in Table 1, below.

As defined above and described herein, R^{u}, R^{v}, R^{w}, R^{x}, R^{x1}, R^{x2}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R:

In some embodiments, one or more of R^{u}, R^{v}, R^{w}, R^{x}, R^{x1}, R^{x2}, R^{y}, and R^{z} are hydrogen. In some embodiments, one or more of R^{u}, R^{v}, R^{w}, R^{x}, R^{x1}, R^{x2}, R^{y}, and R^{z} are R^{A}. In some embodiments, one or more of R^{u}, R^{v}, R^{w}, R^{x}, R^{x1}, R^{x2}, R^{y}, and R^{z} are C₁₋₆alkyl (e.g., methyl, ethyl, isopropyl, etc.). In some embodiments, one or more of R^{u}, R^{v}, R^{w}, R^{x}, R^{x1}, R^{x2}, R^{y}, and R^{z} are C₁₋₆haloalkyl (e.g., —CF₃, —CHF₂, —CH₂F, etc.). In some embodiments, one or more of R^{u}, R^{v}, R^{w}, R^{x}, R^{x1}, R^{x2}, R^{y}, and R^{z} are halogen. In some embodiments, one or more of R^{u}, R^{v}, R^{w}, R^{x}, R^{x1}, R^{x2}, R^{y}, and R^{z} are —CN. In some embodiments, one or more of R^{u}, R^{v}, R^{w}, R^{x}, R^{x1}, R^{x2}, R^{y}, and R^{z} are —NO₂. In some embodiments, one or more of R^{u}, R^{v}, R^{w}, R^{x}, R^{x1}, R^{x2}, R^{y}, and R^{z} are —OR. In some embodiments, one or more of R^{u}, R^{v}, R^{w}, R^{x}, R^{x1}, R^{x2}, R^{y}, and R^{z}, are —SR. In some embodiments, one or more of R^{u}, R^{v}, R^{w}, R^{x}, R^{x1}, R^{x2}, R^{y}, and R^{z} are —N(R)₂. In some embodiments, one or more of R^{u}, R^{v}, R^{w}, R^{x}, R^{x1}, R^{x2}, R^{y}, and R^{z} are —Si(R)₃. In some embodiments, one or more of R^{u}, R^{v}, R^{w}, R^{x}, R^{x1}, R^{x2}, R^{y}, and R^{z} are —S(O)₂R. In some embodiments, one or more of R^{u}, R^{v}, R^{w}, R^{x}, R^{x1}, R^{x2}, R^{y}, and R^{z} are —S(O); N(R)₂. In some embodiments, one or more of R^{u}, R^{v}, R^{w}, R^{x}, R^{x1}, R$2, R^{y}, and R^{z} are —S(O)₂NRC(O)R. In some embodiments, one or more of R^{u}, R^{v}, R^{w}, R^{x}, R^{x1}, R^{x2}, R^{y}, and Rare —S(O)R. In some embodiments, one or more of R^{u}, R^{v}, R^{w}, R^{x}, R^{x1}, R^{x2}, R^{y}, and R^{z} are —S(O)₂OR. In some embodiments, one or more of R^{u}, R^{v}, R^{w}, R^{y}, R^{x1}, R^{x2}, R^{y}, and R^{z} are —C(O)R. In some embodiments, one or more of R^{u}, R^{v}, R^{w}, R^{x}, R^{x1}, R^{x2}, R^{y}, and Rare —C(O)OR. In some embodiments, one or more of R^{u}, R^{v}, R^{w}, R^{x}, R^{x1}, R^{x2}, R^{y}, and R^{z} are —C(O)N(R)₂. In some embodiments, one or more of R^{u}, R^{v}, R^{w}, R^{x}, R^{x1}, R^{x2}, R^{y}, and R^{z} are —C(O)NROR. In some embodiments, one or more of R^{u}, R^{v}, R^{w}, R^{x}, R^{x1}. R$2, R^{y}, and R^{z} are —C(O)NRC(O)R. In some embodiments, one or more of R^{u}, R^{v}, R^{w}, R^{x}, R^{x1}, R^{x2}, R^{y}, and R7 are —C(O)NRS(O)₂R. In some embodiments, one or more of R^{u}, R^{v}, R^{w}, R^{x}, R^{x1}, R^{x2}, R^{y}, and R^{z}, are —OC(O)R. In some embodiments, one or more of R^{u}, R^{v}, R^{w}, R^{x}, R^{x1}, R^{x2}, R^{y}, and R^{z} are —OC(O)N(R)₂. In some embodiments, one or more of R^{u}, R^{v}, R^{w}, R^{x}, R^{x1}, R^{x2}, R^{y}, and R^{z} are —OP(O)(R)₂. In some embodiments, one or more of R^{u}, R^{v}, R^{w}, R^{x}, R^{x1}, R^{x2}, R^{y}, and R^{z} are —OP(O)(OR)₂. In some embodiments, one or more of R^{u}, R^{v}, R^{w}, R^{x}, R^{x1}, R^{x2}, R^{y}, and R7 are —OP(O)(OR)N(R)₂. In some embodiments, one or more of R^{u}, R^{v}, R^{w}, R^{x}, R^{x1}, R^{x2}, R^{y}, and R^{z} are —OP(O)(N(R)₂)₂. In some embodiments, one or more of R^{u}, R^{v}, R^{w}, R^{x}, R^{x1}, R^{x2}. R^{y}, and R^{z} are —NRC(O)OR. In some embodiments, one or more of R^{u}, R^{v}, R^{w}, R^{y}, R^{x1}. R^{x2}, R^{y}, and R^{z} are —NRC(O)R. In some embodiments, one or more of R^{u}, R^{v}, R^{w}, R^{x}, R^{x1}, R^{x2}, R^{y}, and R^{z} are —NRC(O)N(R)₂. In some embodiments, one or more of R^{u}, R^{v}, R^{w}, R^{x}, R^{x1}, R^{x2}, R^{y}, and R^{z} are —NRS(O)₂R. In some embodiments, one or more of R^{u}, R^{v}, R^{w}, R^{x}, R^{x1}, R^{x2}, R^{y}, and R^{z} are —NP(O)(R)₂. In some embodiments, one or more of R^{u}, R^{v}, R^{w}, R^{x}, R^{x1}, R^{x2}, R^{y}, and R^{z} are —NRP(O)(OR)₂. In some embodiments, one or more of R^{u}, R^{v}, R^{w}, R^{x}, R^{x1}, R^{x2}, R^{y}, and R7 are —NRP(O)(OR)N(R)₂. In some embodiments, one or more of R^{u}, R^{v}, R^{w}, R^{x}, R^{x1}, R^{x2}, R^{y}, and R^{z} are —NRP(O)(N(R)₂)₂. In some embodiments, one or more of R^{u}, R^{v}, R^{w}, R^{x}, R^{x1}, R^{x2}, R^{y}, and R7 are —NRS(O)₂R.

In some embodiments, R^{v} is methyl. In some embodiments, R^{v} is —NO₂. In some embodiments, R^{v} is —S(O)₂CF₃.

In some embodiments, R^{w} is chloro.

In some embodiments, R^{x1} is methyl.

In some embodiments, R^{x} is —NO₂. In some embodiments, R^{x} is —S(O)₂CF₃. In some embodiments, R^{x} is

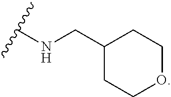 [SMILES: *NCC1CCOCC1]

In some embodiments, Ring R^{x} is

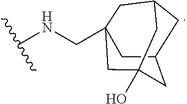 [SMILES: *NCC12CC3CC(CC(O)(C3)C1)C2]

In some embodiments, R^{y} is —CO₂H. In some embodiments, R^{y} is a carboxylic acid isostere known in the art, e.g., Ballatore et al., “Carboxylic acid (bio) isosteres in drug design.” ChemMedChem 2012, 8(3):385.

In some embodiments, R^{z} is fluoro. In some embodiments, R^{z} is acetyl. In some embodiments, R^{z} is

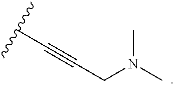 [SMILES: *C#CCN(C)C]

In some embodiments, R^{z} is methyl. In some embodiments, R^{z} is ethyl. In some embodiments, R^{z} is isopropyl. In some embodiments, R^{z} is —CF₂H. In some embodiments, R^{z} is —CH₂NH₂. In some embodiments, R^{z}—(CH₂)₂CO₂H. In some embodiments, R^{z} is —N(Me)iPr. In some embodiments, R^{z} is

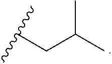 [SMILES: *CC(C)C]

In some embodiments, R^{z} is

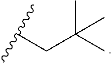 [SMILES: *CC(C)(C)C]

In some embodiments, R^{z} is

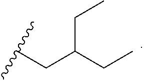 [SMILES: *CC(CC)CC]

In some embodiments, R^{z} is

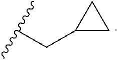 [SMILES: *CC1CC1]

In some embodiments, R^{z} is

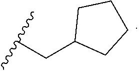 [SMILES: *CC1CCCC1]

In some embodiments, R^{z} is

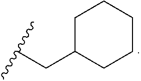 [SMILES: *CC1CCCCC1]

In some embodiments, R^{z} is

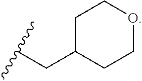 [SMILES: *CC1CCOCC1]

In some embodiments, R^{z} is

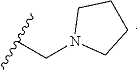 [SMILES: *CN1CCCC1]

In some embodiments, R^{z} is

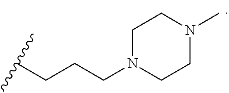 [SMILES: *CCCN1CCN(C)CC1]

In some embodiments, R^{z} is

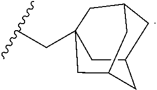 [SMILES: *CC12CC3CC(CC(C3)C1)C2]

In some embodiments, R^{z} is

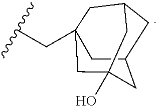 [SMILES: *CC12CC3CC(CC(O)(C3)C1)C2]

In some embodiments, R^{z} is —OH. In some embodiments, R^{z} is -OiPr.

In some embodiments, R^{u}, R^{v}, R^{w}, R^{x}, R$1, R$2, R^{y}, and R^{z} are selected from those depicted in Table 1, below.

As defined above and described herein, each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur.

In some embodiments, R is hydrogen. In some embodiments, R is optionally substituted C₁₋₆ aliphatic. In some embodiments, R is C₁₋₆alkyl (e.g., methyl, ethyl, isopropyl, etc.). In some embodiments, R is C₁₋₆haloalkyl (e.g., —CF₃, —CHF₂, —CH₂F, etc.). In some embodiments, R is optionally substituted phenyl. In some embodiments, R is optionally substituted 3-7 membered saturated or partially unsaturated heterocyclic ring having 1-2 heteroatoms independently selected from nitrogen, oxygen, and sulfur. In some embodiments, R is optionally substituted 5-6 membered heteroaryl ring having 1-4 heteroatoms independently selected from nitrogen, oxygen, and sulfur. In some embodiments, two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur.

In some embodiments, Ring R is selected from those depicted in Table 1, below.

As defined above and described herein, each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is independently an optionally substituted C₁₋₆ aliphatic. In some embodiments, R^{A} is C₁₋₆alkyl (e.g., methyl, ethyl, isopropyl, etc.). In some embodiments, R^{A} is C₁₋₆haloalkyl (e.g., —CF₃, —CHF₂, —CH₂F, etc.). In some embodiments, R^{A} is independently an optionally substituted phenyl. In some embodiments, R^{A} is independently an optionally substituted 3-10 membered saturated or partially unsaturated carbocyclic ring. In some embodiments, R^{A} is independently an optionally substituted 3-7 membered saturated or partially unsaturated heterocyclic ring having 1-2 heteroatoms independently selected from nitrogen, oxygen, and sulfur. In some embodiments, R^{A} is independently an optionally substituted 5-6 membered heteroaryl ring having 1-4 heteroatoms independently selected from nitrogen, oxygen, and sulfur.

In some embodiments, R^{A} is

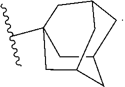 [SMILES: *C12CC3CC(CC(C3)C1)C2]

In some embodiments, Ring R^{A} is

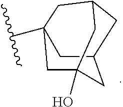 [SMILES: *C12CC3CC(C1)CC(O)(C3)C2]

In some embodiments, R^{A} is selected from those depicted in Table 1, below.

As defined above and described herein, L^{x}, L^{y}, and L^{z} are, independently, a covalent bond or a bivalent, saturated or partially unsaturated, straight or branched C₁-s hydrocarbon chain, wherein 0-3 methylene units of L¹ are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—.

In some embodiments, L¹ is a covalent bond. In some embodiments, L¹ is a covalent bond. In some embodiments, L^{z} is a covalent bond. In some embodiments, L^{x} is a bivalent, saturated or partially unsaturated, straight or branched C₁₋₅ hydrocarbon chain, wherein 0-3 methylene units of L^{x} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—. In some embodiments, L¹ is a bivalent, saturated or partially unsaturated, straight or branched C₁₋₅ hydrocarbon chain, wherein 0-3 methylene units of L^{y} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—. In some embodiments, L^{z} is a bivalent, saturated or partially unsaturated, straight or branched C₁₋₅ hydrocarbon chain, wherein 0-3 methylene units of L^{y} are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—.

In some embodiments, L^{x} is —CH₂—. In some embodiments, L^{x} is

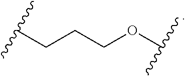 [SMILES: *CCCO*]

In some embodiments, L^{x} is

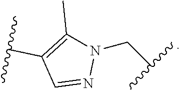 [SMILES: *Cn1ncc(*)c1C]

In some embodiments, L^{x} is

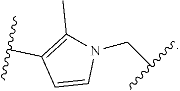 [SMILES: *Cn1ccc(*)c1C]

In some embodiments, L^{x} is

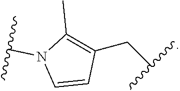 [SMILES: *Cc1ccn(*)c1C]

In some embodiments, L^{x} is

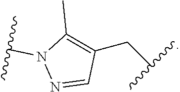 [SMILES: *Cc1cnn(*)c1C]

In some embodiments, L^{x} is

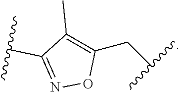 [SMILES: *Cc1onc(*)c1C]

In some embodiments, L^{x} is

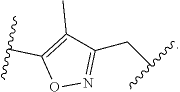 [SMILES: *Cc1noc(*)c1C]

In some embodiments, L^{y} is —CH₂—. In some embodiments, L^{y} is

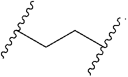 [SMILES: *CC*]

In some embodiments, L^{z} is —CH₂—. In some embodiments, L^{z} is —O—.

In some embodiments, L^{x}, L^{y}, and L^{z} are selected from those depicted in Table 1, below.

As defined above and described herein, X^{a} and X^{b} are, independently, a carbon atom or a nitrogen atom.

In some embodiments, X^{a} is a carbon atom. In some embodiments, X^{a} is a nitrogen atom. In some embodiments, X^{b} is a carbon atom. In some embodiments, X^{b} is a nitrogen atom.

In some embodiments, X^{a} and X^{b} are selected from those depicted in Table 1, below.

As defined above and described herein, s, s′, and s″ are, independently, 0, 1, or 2.

In some embodiments, s is 0. In some embodiments, s′ is 0. In some embodiments, s″ is 0. In some embodiments, s is 1. In some embodiments, s′ is 1. In some embodiments, s″ is 1. In some embodiments, s is 2. In some embodiments, s′ is 2. In some embodiments, s″ is 2.

In some embodiments, s, s′, and s″ are selected from those depicted in Table 1, below.

As defined above and described herein, u, v, w, x, y, and z are, independently, 0, 1, 2, 3, or 4.

In some embodiments, u is 0. In some embodiments, u is 1. In some embodiments, u is 2. In some embodiments, u is 3. In some embodiments, u is 4. In some embodiments, v is 0. In some embodiments, v is 1. In some embodiments, v is 2. In some embodiments, v is 3. In some embodiments, v is 4. In some embodiments, w is 0. In some embodiments, w is 1. In some embodiments, w is 2. In some embodiments, w is 3. In some embodiments, w is 4, some embodiments, x is 0. In some embodiments, x is 1. In some embodiments, x is 2. In some embodiments, x is 3. In some embodiments, x is 4. In some embodiments, y is 0. In some embodiments, y is 1. In some embodiments, y is 2. In some embodiments, y is 3. In some embodiments, y is 4. In some embodiments, z is 0. In some embodiments, z is 1. In some embodiments, z is 2. In some embodiments, z is 3. In some embodiments, z is 4.

In some embodiments, u, v, w, y, and z are selected from those depicted in Table 1, below.

In some embodiments, BBM is

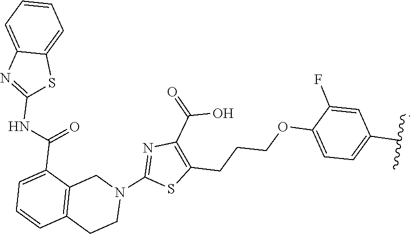 [SMILES: *c1ccc(OCCCc2sc(N3CCc4cccc(C(=O)Nc5nc6ccccc6s5)c4C3)nc2C(=O)O)c(F)c1]

In some embodiments, BBM is

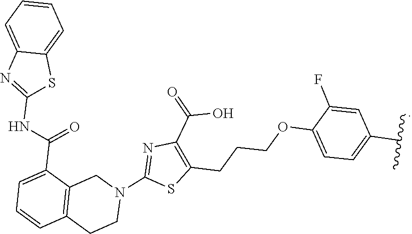 [SMILES: *c1ccc(OCCCc2sc(N3CCc4cccc(C(=O)Nc5nc6ccccc6s5)c4C3)nc2C(=O)O)c(F)c1]

In some embodiments, BBM is

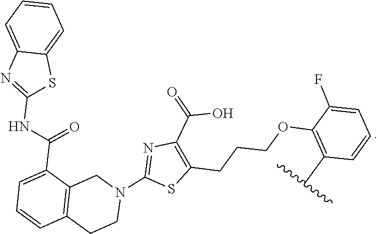 [SMILES: *c1cccc(F)c1OCCCc1sc(N2CCc3cccc(C(=O)Nc4nc5ccccc5s4)c3C2)nc1C(=O)O]

In some embodiments, BBM is

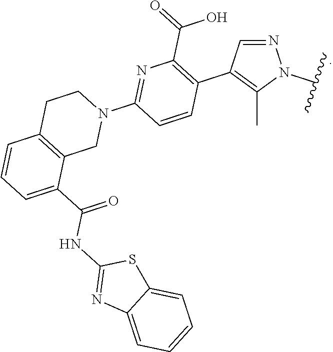 [SMILES: *n1ncc(-c2ccc(N3CCc4cccc(C(=O)Nc5nc6ccccc6s5)c4C3)nc2C(=O)O)c1C]

In some embodiments, BBM is

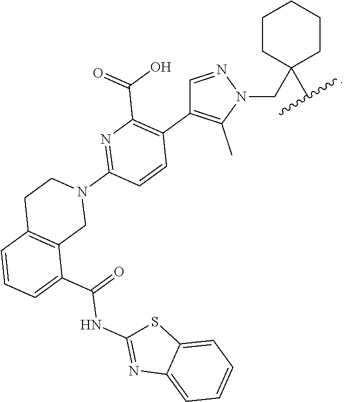 [SMILES: *C1(Cn2ncc(-c3ccc(N4CCc5cccc(C(=O)Nc6nc7ccccc7s6)c5C4)nc3C(=O)O)c2C)CCCCC1]

In some embodiments, BBM is

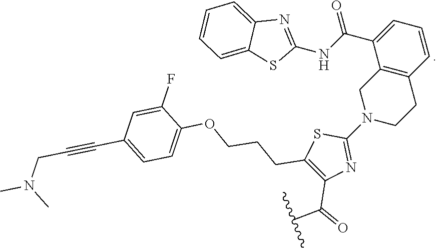 [SMILES: *C(=O)c1nc(N2CCc3cccc(C(=O)Nc4nc5ccccc5s4)c3C2)sc1CCCOc1ccc(C#CCN(C)C)cc1F]

In some embodiments, BBM is

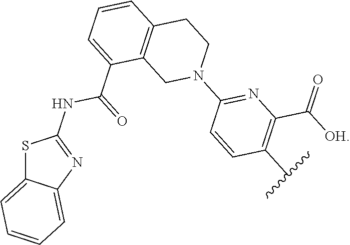 [SMILES: *c1ccc(N2CCc3cccc(C(=O)Nc4nc5ccccc5s4)c3C2)nc1C(=O)O]

In some embodiments, BBM is

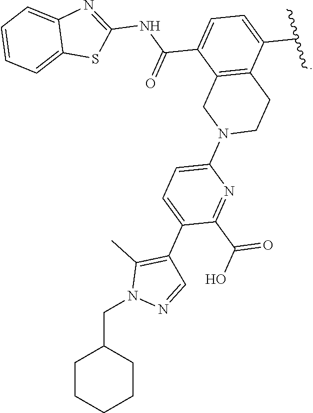 [SMILES: *c1ccc(C(=O)Nc2nc3ccccc3s2)c2c1CCN(c1ccc(-c3cnn(CC4CCCCC4)c3C)c(C(=O)O)n1)C2]

In some embodiments, BBM is

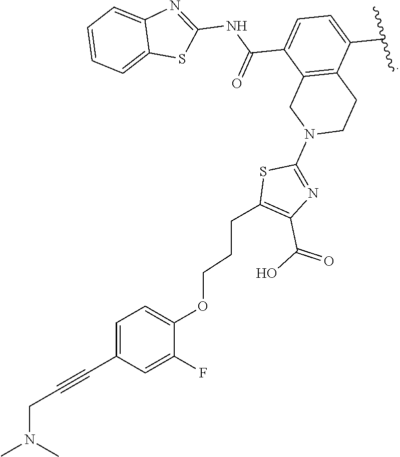 [SMILES: *c1ccc(C(=O)Nc2nc3ccccc3s2)c2c1CCN(c1nc(C(=O)O)c(CCCOc3ccc(C#CCN(C)C)cc3F)s1)C2]

In some embodiments, BBM is

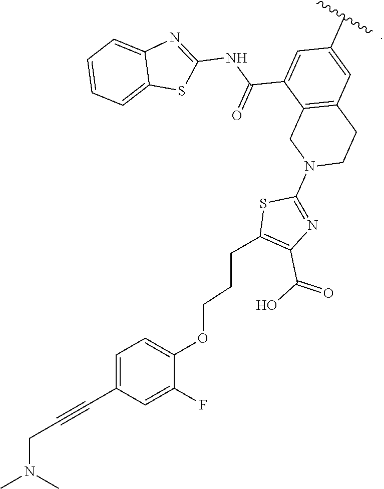 [SMILES: *c1cc2c(c(C(=O)Nc3nc4ccccc4s3)c1)CN(c1nc(C(=O)O)c(CCCOc3ccc(C#CCN(C)C)cc3F)s1)CC2]

In some embodiments, BBM is

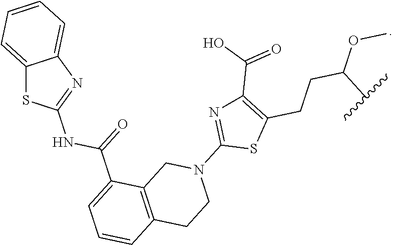 [SMILES: *C(CCc1sc(N2CCc3cccc(C(=O)Nc4nc5ccccc5s4)c3C2)nc1C(=O)O)OC]

In some embodiments, BBM is

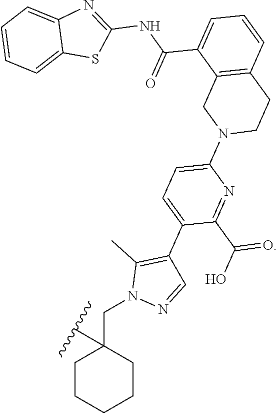 [SMILES: *C1(Cn2ncc(-c3ccc(N4CCc5cccc(C(=O)Nc6nc7ccccc7s6)c5C4)nc3C(=O)O)c2C)CCCCC1]

In some embodiments, BBM is

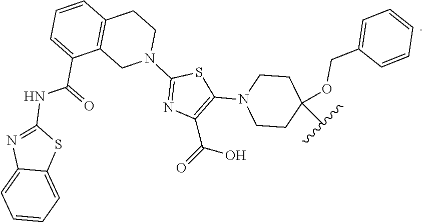 [SMILES: *C1(OCc2ccccc2)CCN(c2sc(N3CCc4cccc(C(=O)Nc5nc6ccccc6s5)c4C3)nc2C(=O)O)CC1]

In some embodiments, BBM is

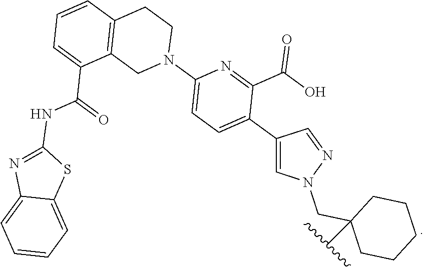 [SMILES: *C1(Cn2cc(-c3ccc(N4CCc5cccc(C(=O)Nc6nc7ccccc7s6)c5C4)nc3C(=O)O)cn2)CCCCC1]

In some embodiments, BBM is

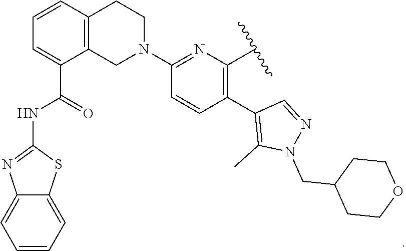 [SMILES: *c1nc(N2CCc3cccc(C(=O)Nc4nc5ccccc5s4)c3C2)ccc1-c1cnn(CC2CCOCC2)c1C]

In some embodiments, BBM is

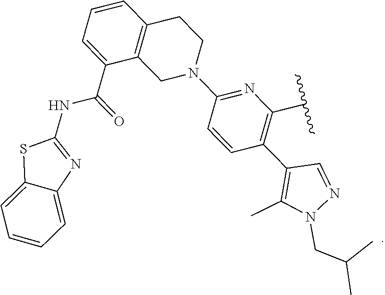 [SMILES: *c1nc(N2CCc3cccc(C(=O)Nc4nc5ccccc5s4)c3C2)ccc1-c1cnn(CC(C)C)c1C]

In some embodiments, BBM is

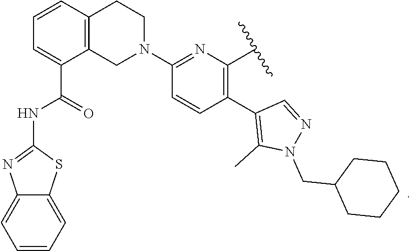 [SMILES: *c1nc(N2CCc3cccc(C(=O)Nc4nc5ccccc5s4)c3C2)ccc1-c1cnn(CC2CCCCC2)c1C]

In some embodiments, BBM is

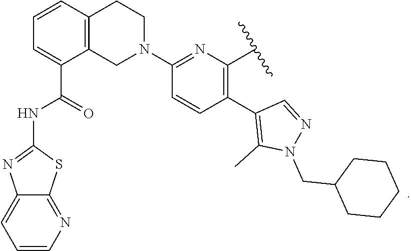 [SMILES: *c1nc(N2CCc3cccc(C(=O)Nc4nc5cccnc5s4)c3C2)ccc1-c1cnn(CC2CCCCC2)c1C]

In some embodiments, BBM is

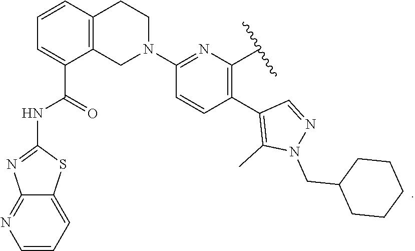 [SMILES: *c1nc(N2CCc3cccc(C(=O)Nc4nc5ncccc5s4)c3C2)ccc1-c1cnn(CC2CCCCC2)c1C]

In some embodiments, BBM is

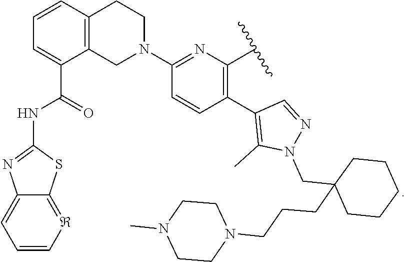

In some embodiments, BBM is

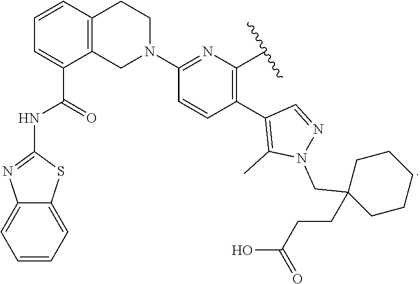 [SMILES: *c1nc(N2CCc3cccc(C(=O)Nc4nc5ccccc5s4)c3C2)ccc1-c1cnn(CC2(CCC(=O)O)CCCCC2)c1C]

In some embodiments, BBM is

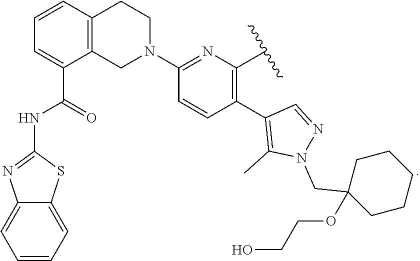 [SMILES: *c1nc(N2CCc3cccc(C(=O)Nc4nc5ccccc5s4)c3C2)ccc1-c1cnn(CC2(OCCO)CCCCC2)c1C]

In some embodiments, BBM is

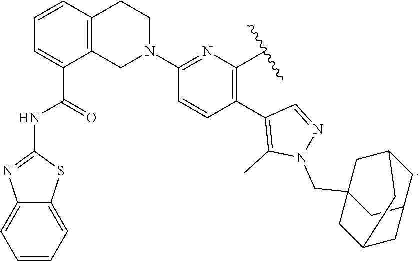 [SMILES: *c1nc(N2CCc3cccc(C(=O)Nc4nc5ccccc5s4)c3C2)ccc1-c1cnn(CC23CC4CC(CC(C4)C2)C3)c1C]

In some embodiments, BBM is

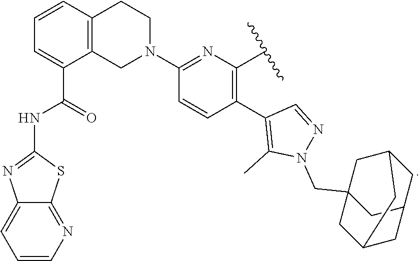 [SMILES: *c1nc(N2CCc3cccc(C(=O)Nc4nc5cccnc5s4)c3C2)ccc1-c1cnn(CC23CC4CC(CC(C4)C2)C3)c1C]

In some embodiments, BBM is

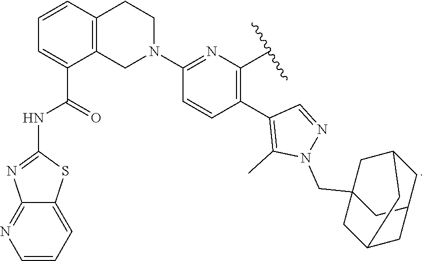 [SMILES: *c1nc(N2CCc3cccc(C(=O)Nc4nc5ncccc5s4)c3C2)ccc1-c1cnn(CC23CC4CC(CC(C4)C2)C3)c1C]

In some embodiments, BBM is

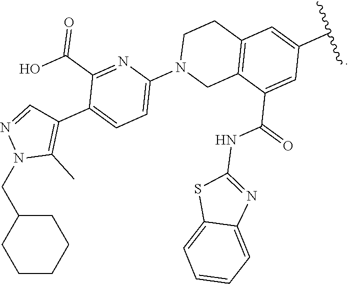 [SMILES: *c1cc2c(c(C(=O)Nc3nc4ccccc4s3)c1)CN(c1ccc(-c3cnn(CC4CCCCC4)c3C)c(C(=O)O)n1)CC2]

In some embodiments, BBM is

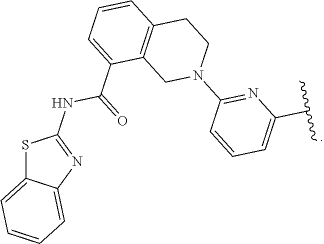 [SMILES: *c1cccc(N2CCc3cccc(C(=O)Nc4nc5ccccc5s4)c3C2)n1]

In some embodiments, BBM is

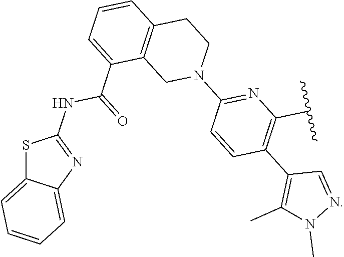 [SMILES: *c1nc(N2CCc3cccc(C(=O)Nc4nc5ccccc5s4)c3C2)ccc1-c1cnn(C)c1C]

In some embodiments, BBM is

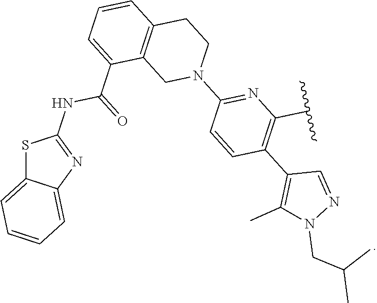 [SMILES: *c1nc(N2CCc3cccc(C(=O)Nc4nc5ccccc5s4)c3C2)ccc1-c1cnn(CC(C)C)c1C]

In some embodiments, BBM is

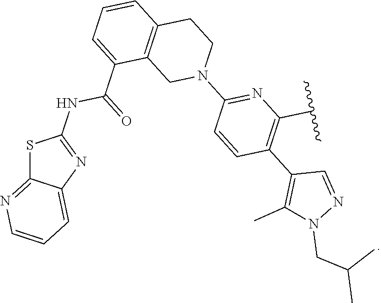 [SMILES: *c1nc(N2CCc3cccc(C(=O)Nc4nc5cccnc5s4)c3C2)ccc1-c1cnn(CC(C)C)c1C]

In some embodiments, BBM is

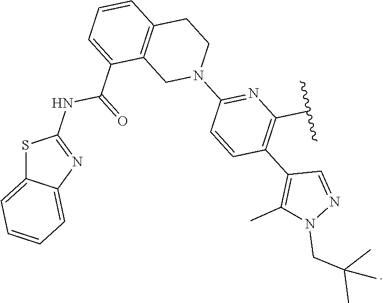 [SMILES: *c1nc(N2CCc3cccc(C(=O)Nc4nc5ccccc5s4)c3C2)ccc1-c1cnn(CC(C)(C)C)c1C]

In some embodiments, BBM is

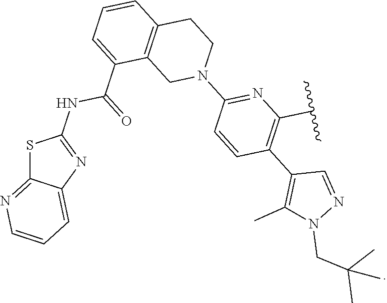 [SMILES: *c1nc(N2CCc3cccc(C(=O)Nc4nc5cccnc5s4)c3C2)ccc1-c1cnn(CC(C)(C)C)c1C]

In some embodiments, BBM is

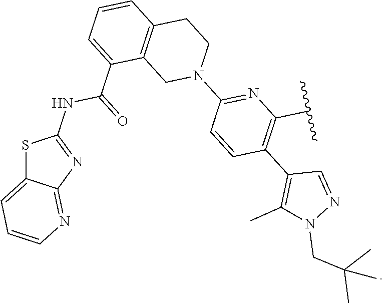 [SMILES: *c1nc(N2CCc3cccc(C(=O)Nc4nc5ncccc5s4)c3C2)ccc1-c1cnn(CC(C)(C)C)c1C]

In some embodiments, BBM is

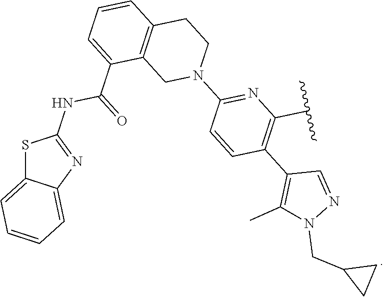 [SMILES: *c1nc(N2CCc3cccc(C(=O)Nc4nc5ccccc5s4)c3C2)ccc1-c1cnn(CC2CC2)c1C]

In some embodiments, BBM is

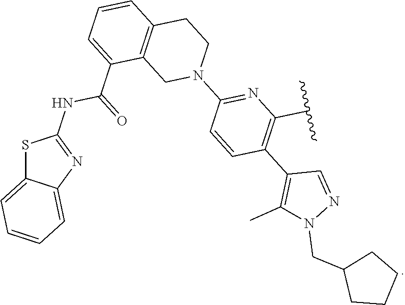 [SMILES: *c1nc(N2CCc3cccc(C(=O)Nc4nc5ccccc5s4)c3C2)ccc1-c1cnn(CC2CCCC2)c1C]

In some embodiments, BBM is

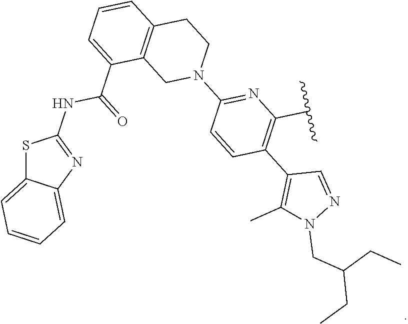 [SMILES: *c1nc(N2CCc3cccc(C(=O)Nc4nc5ccccc5s4)c3C2)ccc1-c1cnn(CC(CC)CC)c1C]

In some embodiments, BBM is

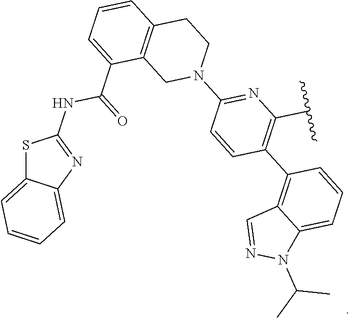 [SMILES: *c1nc(N2CCc3cccc(C(=O)Nc4nc5ccccc5s4)c3C2)ccc1-c1cccc2c1cnn2C(C)C]

In some embodiments, BBM is

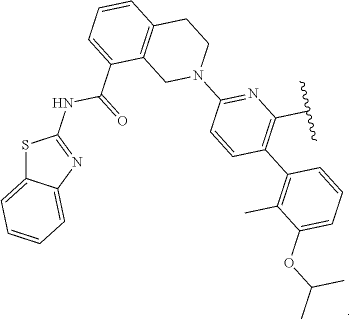 [SMILES: *c1nc(N2CCc3cccc(C(=O)Nc4nc5ccccc5s4)c3C2)ccc1-c1cccc(OC(C)C)c1C]

In some embodiments, BBM is

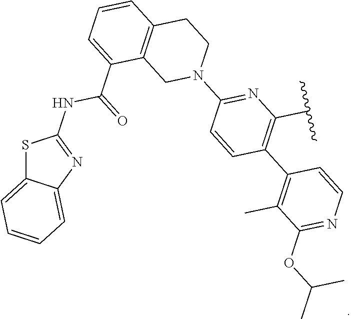 [SMILES: *c1nc(N2CCc3cccc(C(=O)Nc4nc5ccccc5s4)c3C2)ccc1-c1ccnc(OC(C)C)c1C]

In some embodiments, BBM is

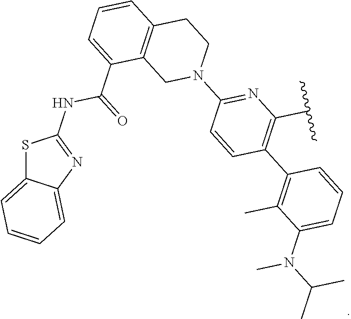 [SMILES: *c1nc(N2CCc3cccc(C(=O)Nc4nc5ccccc5s4)c3C2)ccc1-c1cccc(N(C)C(C)C)c1C]

In some embodiments, BBM is

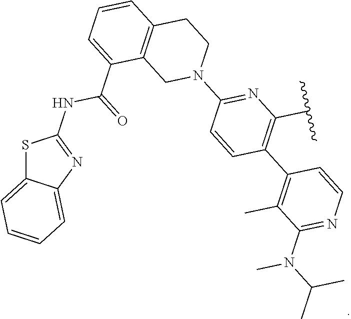 [SMILES: *c1nc(N2CCc3cccc(C(=O)Nc4nc5ccccc5s4)c3C2)ccc1-c1ccnc(N(C)C(C)C)c1C]

In some embodiments, BBM is

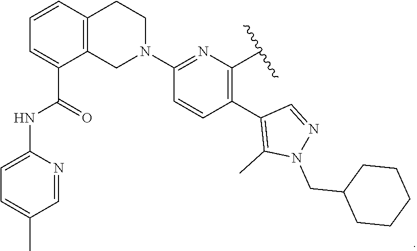 [SMILES: *c1nc(N2CCc3cccc(C(=O)Nc4ccc(C)cn4)c3C2)ccc1-c1cnn(CC2CCCCC2)c1C]

In some embodiments, BBM is

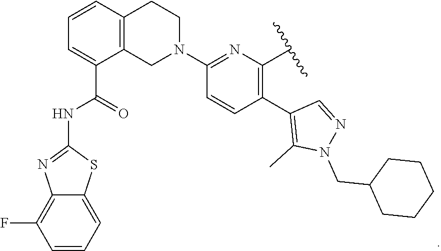 [SMILES: *c1nc(N2CCc3cccc(C(=O)Nc4nc5c(F)cccc5s4)c3C2)ccc1-c1cnn(CC2CCCCC2)c1C]

In some embodiments, BBM is

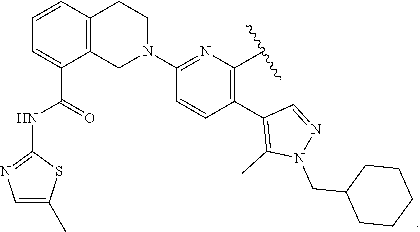 [SMILES: *c1nc(N2CCc3cccc(C(=O)Nc4ncc(C)s4)c3C2)ccc1-c1cnn(CC2CCCCC2)c1C]

In some embodiments, BBM is

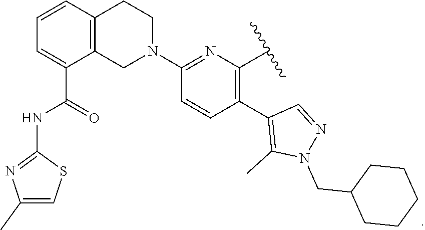 [SMILES: *c1nc(N2CCc3cccc(C(=O)Nc4nc(C)cs4)c3C2)ccc1-c1cnn(CC2CCCCC2)c1C]

In some embodiments, BBM is

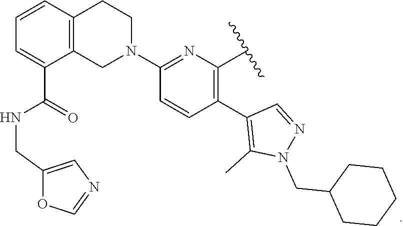 [SMILES: *c1nc(N2CCc3cccc(C(=O)NCc4cnco4)c3C2)ccc1-c1cnn(CC2CCCCC2)c1C]

In some embodiments, BBM is

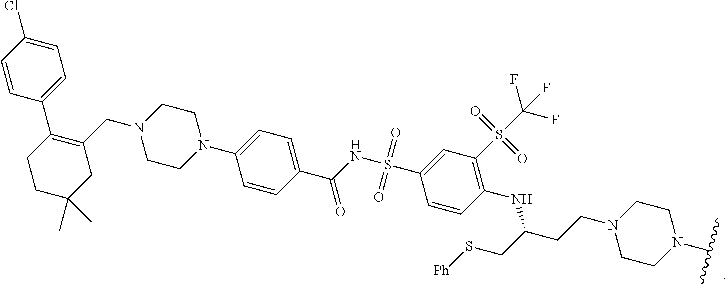 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

In some embodiments, BBM is

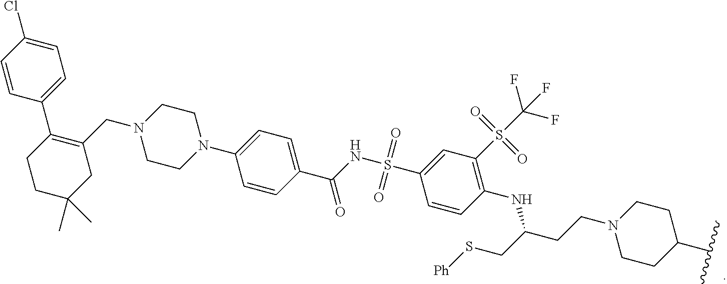 [SMILES: *C1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

In some embodiments, BBM is

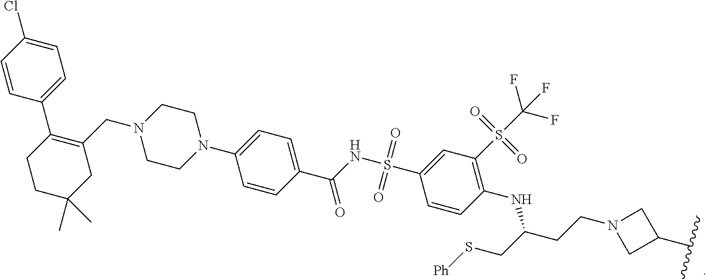 [SMILES: *C1CN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)C1]

In some embodiments, BBM is

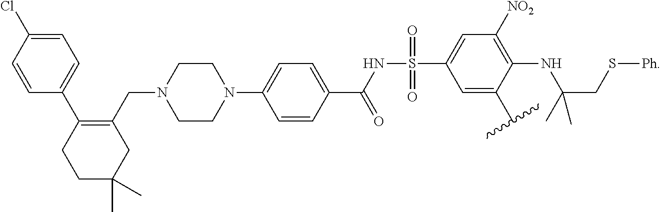 [SMILES: *c1cc(S(=O)(=O)NC(=O)c2ccc(N3CCN(CC4=C(c5ccc(Cl)cc5)CCC(C)(C)C4)CC3)cc2)cc([N+](=O)[O-])c1NC(C)(C)CSc1ccccc1]

In some embodiments, BBM is

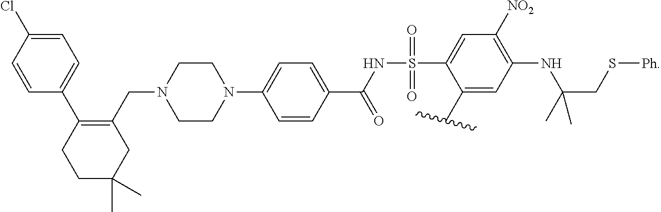 [SMILES: *c1cc(NC(C)(C)CSc2ccccc2)c([N+](=O)[O-])cc1S(=O)(=O)NC(=O)c1ccc(N2CCN(CC3=C(c4ccc(Cl)cc4)CCC(C)(C)C3)CC2)cc1]

In some embodiments, BBM is

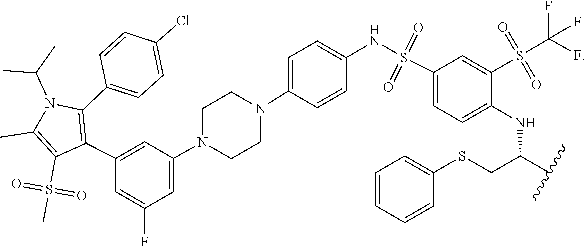 [SMILES: *[C@H](CSc1ccccc1)Nc1ccc(S(=O)(=O)Nc2ccc(N3CCN(c4cc(F)cc(-c5c(S(C)(=O)=O)c(C)n(C(C)C)c5-c5ccc(Cl)cc5)c4)CC3)cc2)cc1S(=O)(=O)C(F)(F)F]

In some embodiments, BBM is

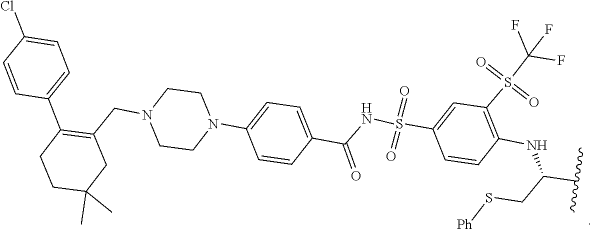 [SMILES: *[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

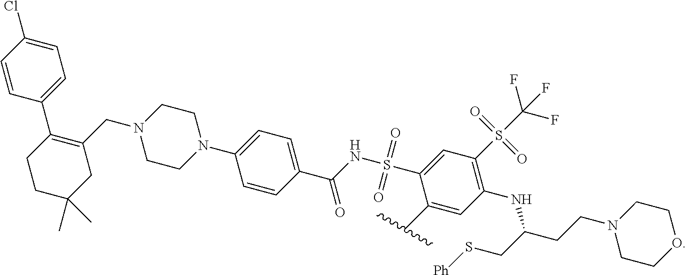 [SMILES: *c1cc(N[C@H](CCN2CCOCC2)CSc2ccccc2)c(S(=O)(=O)C(F)(F)F)cc1S(=O)(=O)NC(=O)c1ccc(N2CCN(CC3=C(c4ccc(Cl)cc4)CCC(C)(C)C3)CC2)cc1]

In some embodiments, BBM is

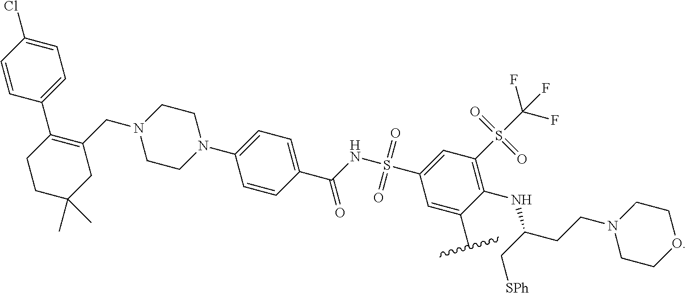

In some embodiments, BBM is

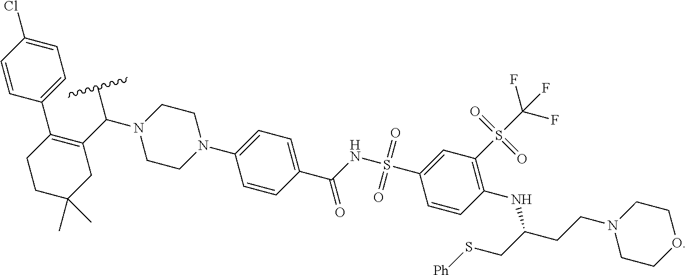 [SMILES: *C(C1=C(c2ccc(Cl)cc2)CCC(C)(C)C1)N1CCN(c2ccc(C(=O)NS(=O)(=O)c3ccc(N[C@H](CCN4CCOCC4)CSc4ccccc4)c(S(=O)(=O)C(F)(F)F)c3)cc2)CC1]

In some embodiments, BBM is

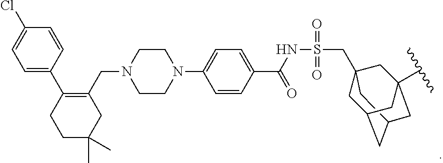 [SMILES: *C12CC3CC(C1)CC(CS(=O)(=O)NC(=O)c1ccc(N4CCN(CC5=C(c6ccc(Cl)cc6)CCC(C)(C)C5)CC4)cc1)(C3)C2]

In some embodiments, BBM is

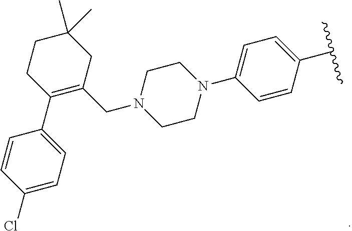 [SMILES: *c1ccc(N2CCN(CC3=C(c4ccc(Cl)cc4)CCC(C)(C)C3)CC2)cc1]

In some embodiments, BBM is

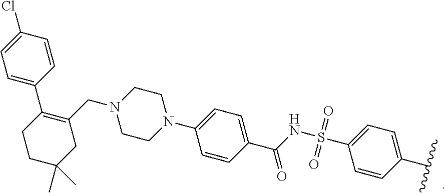 [SMILES: *c1ccc(S(=O)(=O)NC(=O)c2ccc(N3CCN(CC4=C(c5ccc(Cl)cc5)CCC(C)(C)C4)CC3)cc2)cc1]

In some embodiments, BBM is

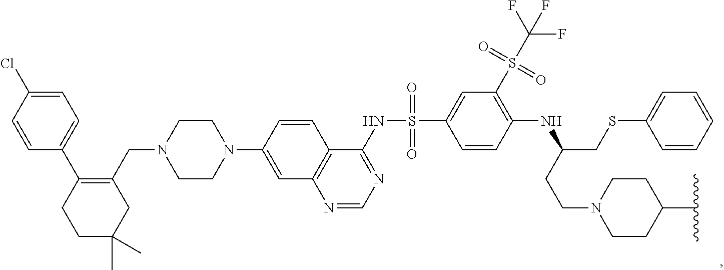 [SMILES: *C1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2S(=O)(=O)C(F)(F)F)CC1]

In some embodiments, BBM is

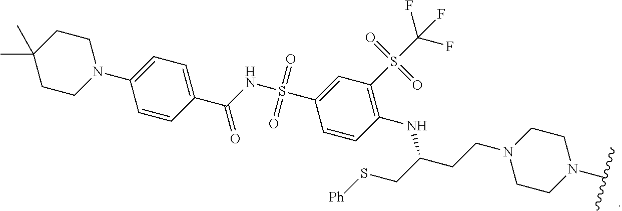 [SMILES: *N1CCN(CC[C@H](CSc2ccccc2)Nc2ccc(S(=O)(=O)NC(=O)c3ccc(N4CCC(C)(C)CC4)cc3)cc2S(=O)(=O)C(F)(F)F)CC1]

In some embodiments, BBM is

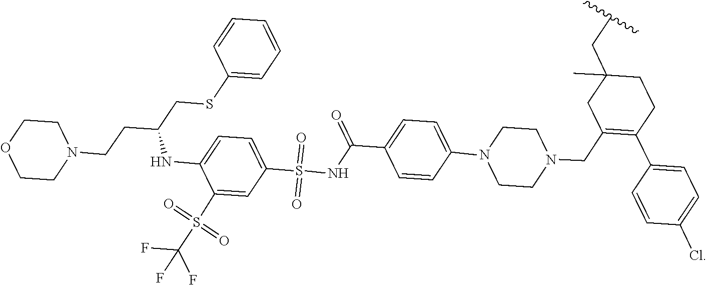 [SMILES: *CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, BBM is

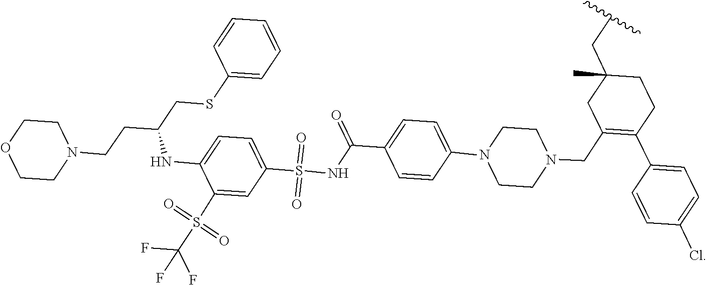 [SMILES: *C[C@]1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, BBM is

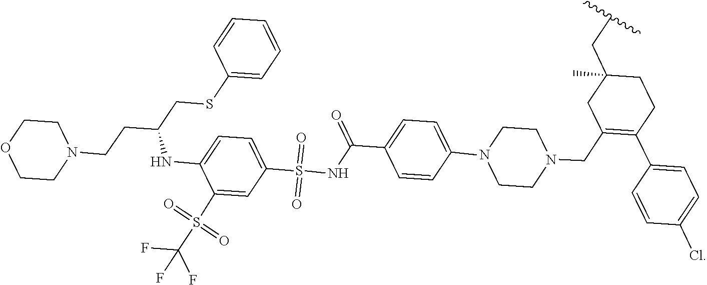 [SMILES: *C[C@@]1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, BBM is

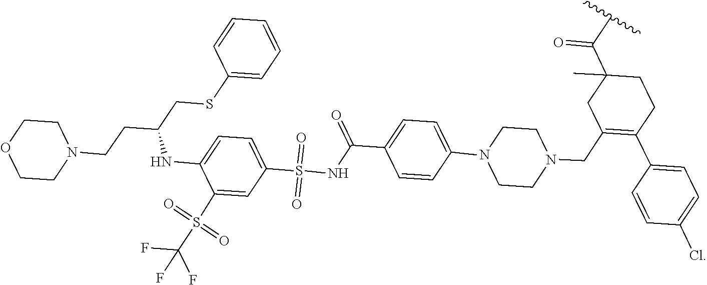 [SMILES: *C(=O)C1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, BBM is

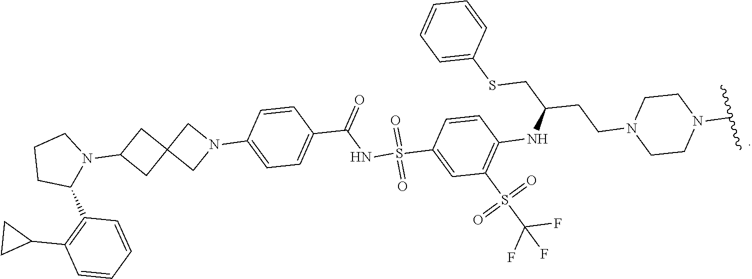 [SMILES: *N1CCN(CC[C@H](CSc2ccccc2)Nc2ccc(S(=O)(=O)NC(=O)c3ccc(N4CC5(CC(N6CCC[C@H]6c6ccccc6C6CC6)C5)C4)cc3)cc2S(=O)(=O)C(F)(F)F)CC1]

In some embodiments, BBM is

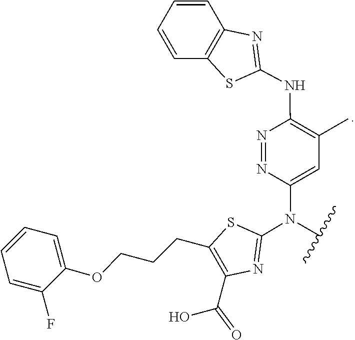 [SMILES: *N(c1cc(C)c(Nc2nc3ccccc3s2)nn1)c1nc(C(=O)O)c(CCCOc2ccccc2F)s1]

In some embodiments, BBM is

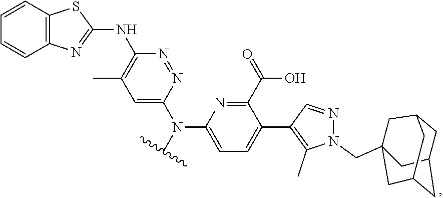 [SMILES: *N(c1cc(C)c(Nc2nc3ccccc3s2)nn1)c1ccc(-c2cnn(CC34CC5CC(CC(C5)C3)C4)c2C)c(C(=O)O)n1]

In some embodiments, BBM is

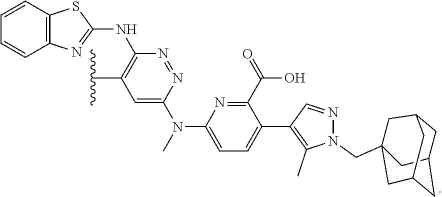 [SMILES: *c1cc(N(C)c2ccc(-c3cnn(CC45CC6CC(CC(C6)C4)C5)c3C)c(C(=O)O)n2)nnc1Nc1nc2ccccc2s1]

In some embodiments, BBM is

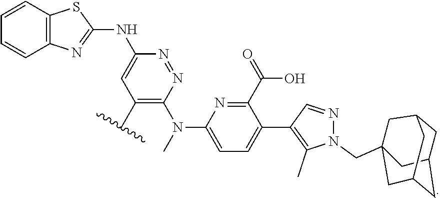 [SMILES: *c1cc(Nc2nc3ccccc3s2)nnc1N(C)c1ccc(-c2cnn(CC34CC5CC(CC(C5)C3)C4)c2C)c(C(=O)O)n1]

In some embodiments, BBM is

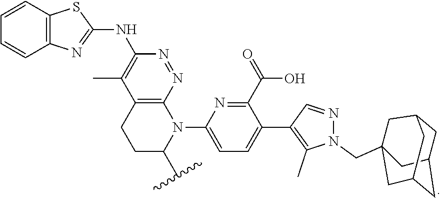 [SMILES: *C1CCc2c(nnc(Nc3nc4ccccc4s3)c2C)N1c1ccc(-c2cnn(CC34CC5CC(CC(C5)C3)C4)c2C)c(C(=O)O)n1]

In some embodiments, BBM is

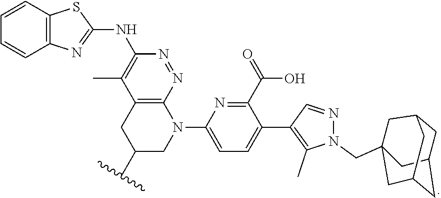 [SMILES: *C1Cc2c(nnc(Nc3nc4ccccc4s3)c2C)N(c2ccc(-c3cnn(CC45CC6CC(CC(C6)C4)C5)c3C)c(C(=O)O)n2)C1]

In some embodiments, BBM is

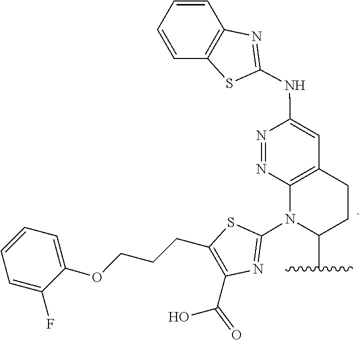 [SMILES: *C1CCc2cc(Nc3nc4ccccc4s3)nnc2N1c1nc(C(=O)O)c(CCCOc2ccccc2F)s1]

In some embodiments, BBM is

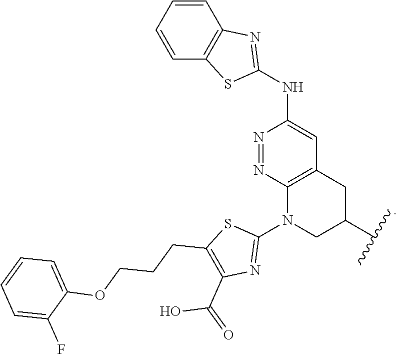 [SMILES: *C1Cc2cc(Nc3nc4ccccc4s3)nnc2N(c2nc(C(=O)O)c(CCCOc3ccccc3F)s2)C1]

In some embodiments, BBM is

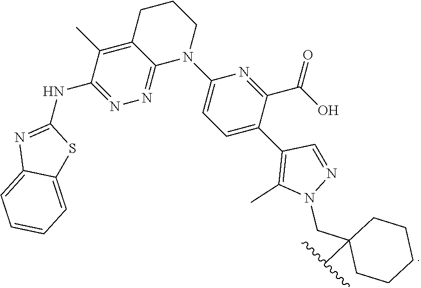 [SMILES: *C1(Cn2ncc(-c3ccc(N4CCCc5c4nnc(Nc4nc6ccccc6s4)c5C)nc3C(=O)O)c2C)CCCCC1]

In some embodiments, BBM is

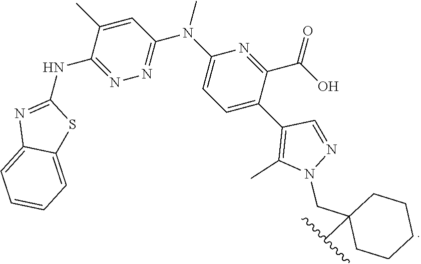 [SMILES: *C1(Cn2ncc(-c3ccc(N(C)c4cc(C)c(Nc5nc6ccccc6s5)nn4)nc3C(=O)O)c2C)CCCCC1]

In some embodiments, BBM is

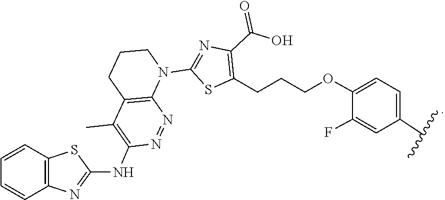 [SMILES: *c1ccc(OCCCc2sc(N3CCCc4c3nnc(Nc3nc5ccccc5s3)c4C)nc2C(=O)O)c(F)c1]

In some embodiments, BBM is

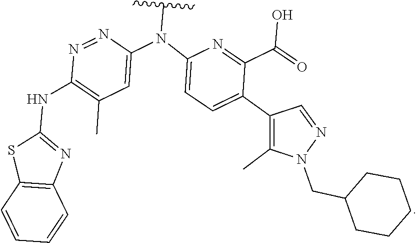 [SMILES: *N(c1cc(C)c(Nc2nc3ccccc3s2)nn1)c1ccc(-c2cnn(CC3CCCCC3)c2C)c(C(=O)O)n1]

In some embodiments, BBM is

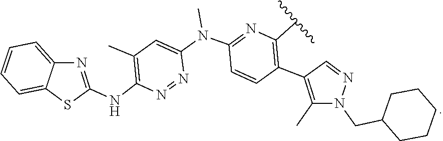 [SMILES: *c1nc(N(C)c2cc(C)c(Nc3nc4ccccc4s3)nn2)ccc1-c1cnn(CC2CCCCC2)c1C]

In some embodiments, BBM is

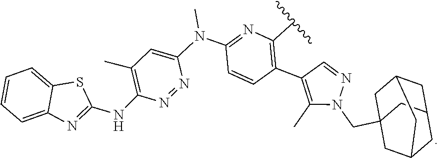 [SMILES: *c1nc(N(C)c2cc(C)c(Nc3nc4ccccc4s3)nn2)ccc1-c1cnn(CC23CC4CC(CC(C4)C2)C3)c1C]

In some embodiments, BBM is

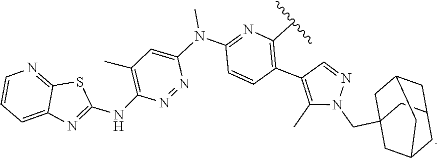 [SMILES: *c1nc(N(C)c2cc(C)c(Nc3nc4cccnc4s3)nn2)ccc1-c1cnn(CC23CC4CC(CC(C4)C2)C3)c1C]

In some embodiments, BBM is

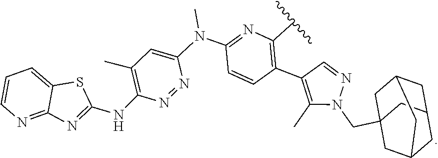 [SMILES: *c1nc(N(C)c2cc(C)c(Nc3nc4ncccc4s3)nn2)ccc1-c1cnn(CC23CC4CC(CC(C4)C2)C3)c1C]

In some embodiments, BBM is

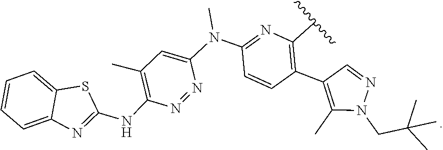 [SMILES: *c1nc(N(C)c2cc(C)c(Nc3nc4ccccc4s3)nn2)ccc1-c1cnn(CC(C)(C)C)c1C]

in some embodiments, BBM is

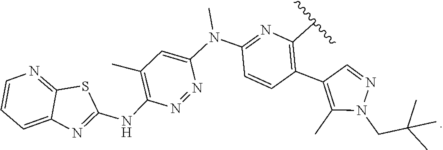 [SMILES: *c1nc(N(C)c2cc(C)c(Nc3nc4cccnc4s3)nn2)ccc1-c1cnn(CC(C)(C)C)c1C]

In some embodiments, BBM is

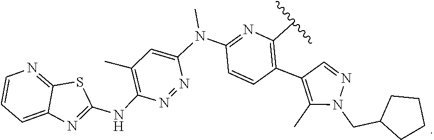 [SMILES: *c1nc(N(C)c2cc(C)c(Nc3nc4cccnc4s3)nn2)ccc1-c1cnn(CC2CCCC2)c1C]

In some embodiments, BBM is

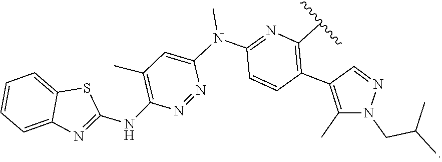 [SMILES: *c1nc(N(C)c2cc(C)c(Nc3nc4ccccc4s3)nn2)ccc1-c1cnn(CC(C)C)c1C]

In some embodiments, BBM is

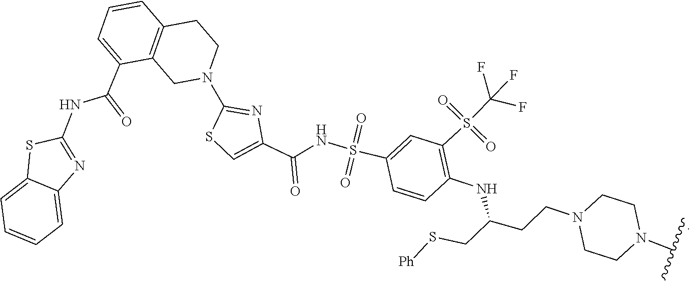 [SMILES: *N1CCN(CC[C@H](CSc2ccccc2)Nc2ccc(S(=O)(=O)NC(=O)c3csc(N4CCc5cccc(C(=O)Nc6nc7ccccc7s6)c5C4)n3)cc2S(=O)(=O)C(F)(F)F)CC1]

In some embodiments, BBM is

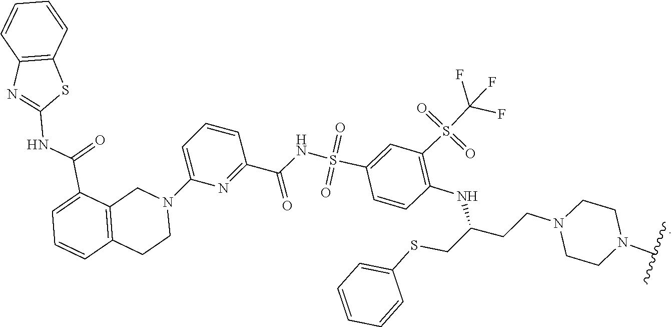 [SMILES: *N1CCN(CC[C@H](CSc2ccccc2)Nc2ccc(S(=O)(=O)NC(=O)c3cccc(N4CCc5cccc(C(=O)Nc6nc7ccccc7s6)c5C4)n3)cc2S(=O)(=O)C(F)(F)F)CC1]

In some embodiments, the present invention provides a compound of formula I, wherein BBM is AZD-4320 thereby forming a compound of formula I-ee:

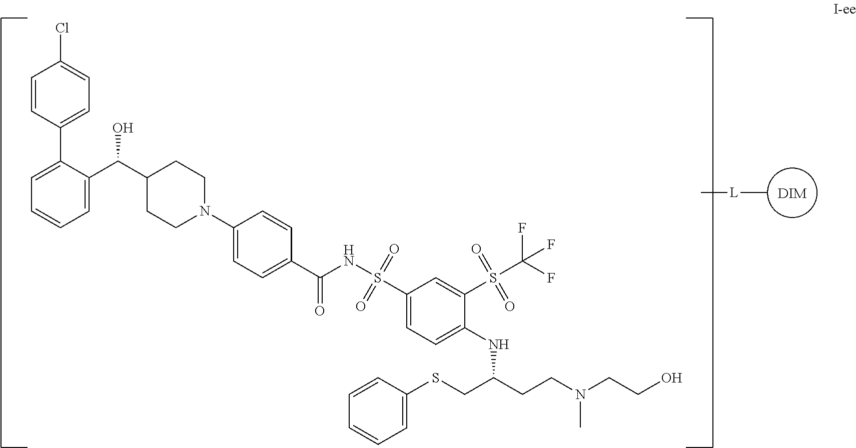

or a pharmaceutically acceptable salt, wherein L and DIM are as defined above and described in embodiments herein.

### Ligase Binding Moiety (LBM)

In some embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, WO 2017/197046, WO 2017/197051, WO 2017/197055, and WO 2017/197056, the entirety of each of which is herein incorporated by reference.

As defined herein and described below, wherein a formula is depicted using square brackets, e.g.,

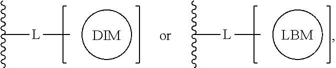

L is attached to a modifiable carbon, oxygen, or nitrogen atom within DIM or LBM including substitution or replacement of a defined group in DIM or LBM.

In certain embodiments, the present invention provides a compound of formula I, wherein LBM is an IMiD-based (immunomodulatory imide drug-based) cereblon E3 ubiquitin ligase binding moiety thereby forming a compound of formula I-a-1, I-a-2, I-a-3, I-a-4, I-a-5, I-a-6, I-a-7, I-a-8, I-a-9, or I-a-10 respectively:

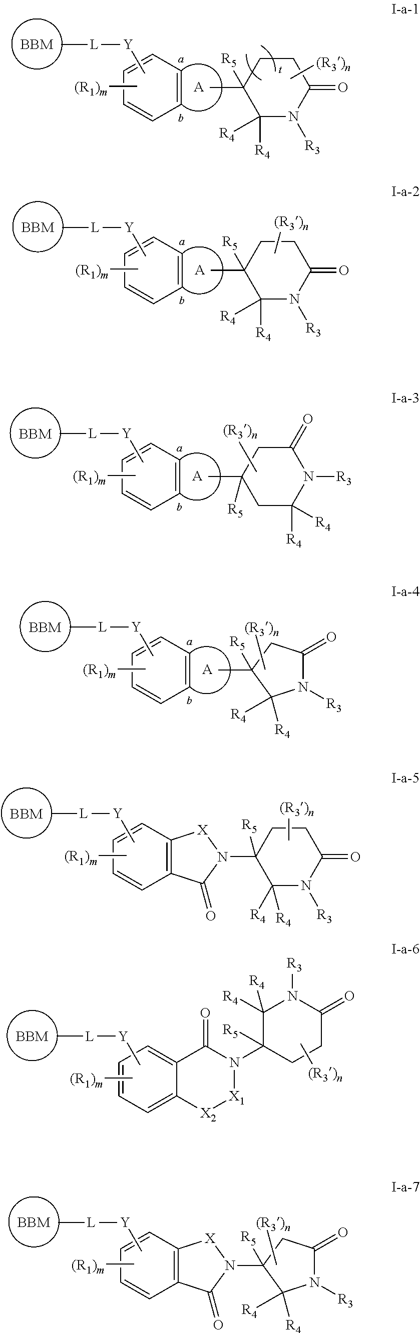

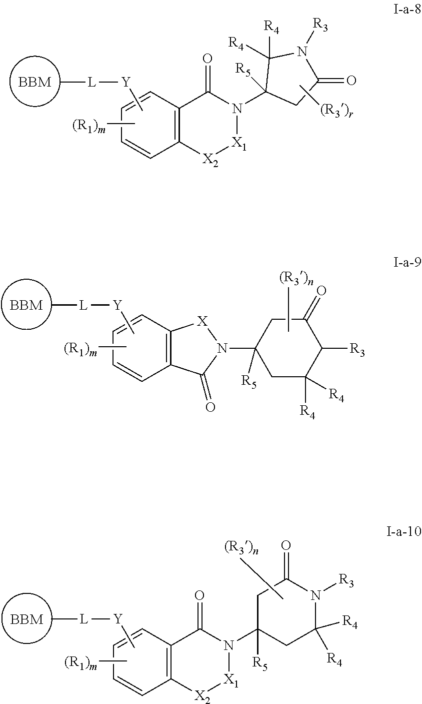

or a compound of formula I-a′-1, I-a′-2, I-a′-3, I-a′-4, I-a′-5, I-a′-6, I-a′-7, I-a′-8, I-a′-9, or I-a′-10 respectively:

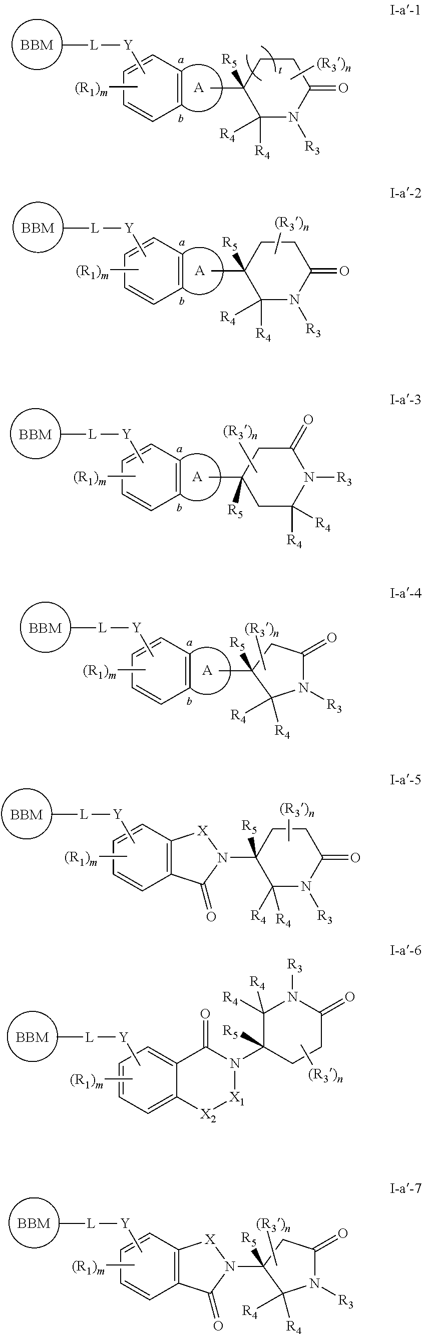

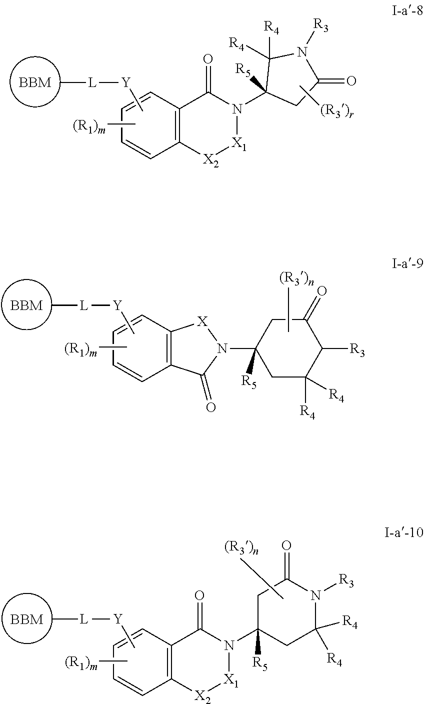

or a compound of formula I-a″-1. I-a″-2. I-a″-3. I-a″-4. I-a″-5. I-a″-6. I-a″-7. I-a″-8. I-a″-9, or I-a″-10 respectively:

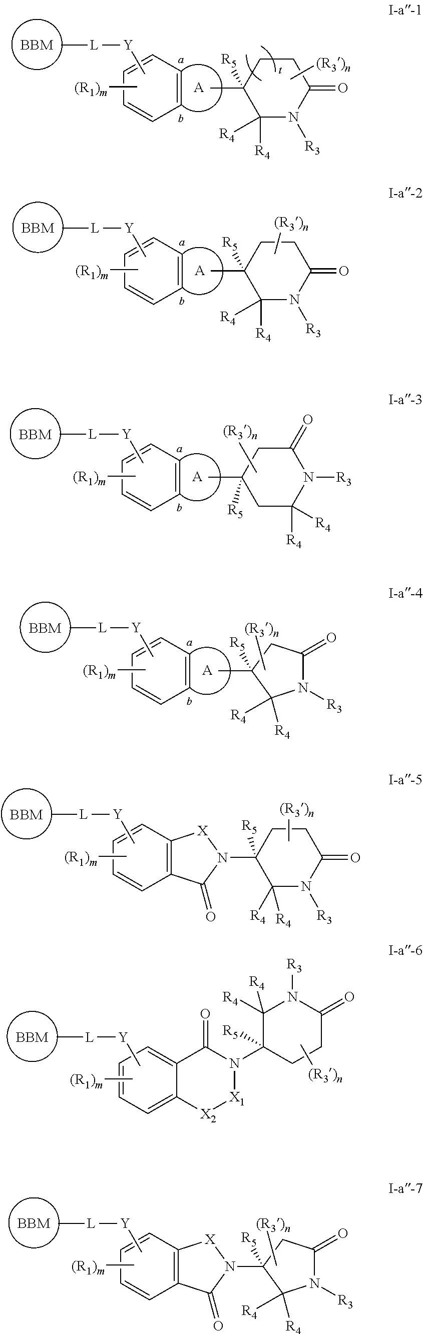

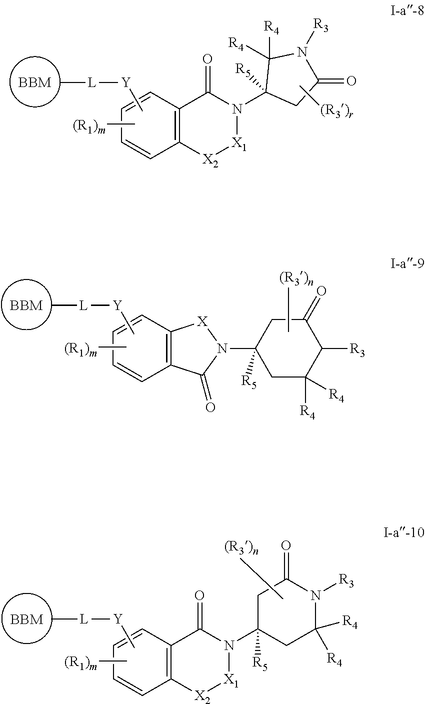

or a pharmaceutically acceptable salt thereof, wherein L and BBM are as defined above and described in embodiments herein, and wherein:

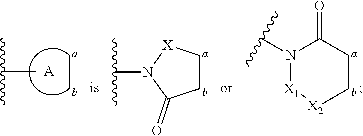

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁—X₂ is C(R₃)═N or C(R₃)₂—C(R₃)₂;
each R¹ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R^{a} is independently H or C₁-C₃ alkyl optionally substituted with C₆-C₁₀ aryl or 5- to 10-membered heteroaryl;
each Rₐ′ is independently C₁-C₃ alkyl;
each R^{a} is independently H or C₁-C₃ alkyl: or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or C₁;
each Rₐ independently is H or C₁-C₆ alkyl;
Rb is H or tosyl;
t is 0 or 1;
m is 0, 1, 2 or 3; and
n is 0, 1 or 2.

In some embodiments, LBM is

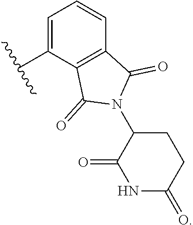 [SMILES: *c1cccc2c1C(=O)N(C1CCC(=O)NC1=O)C2=O]

In some embodiments, LBM is

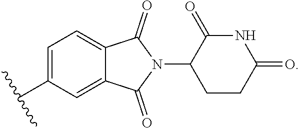 [SMILES: *c1ccc2c(c1)C(=O)N(C1CCC(=O)NC1=O)C2=O]

In some embodiments, LBM is

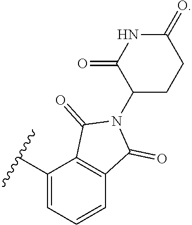 [SMILES: *c1cccc2c1C(=O)N(C1CCC(=O)NC1=O)C2=O]

In some embodiments, LBM is

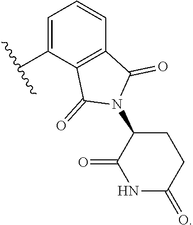 [SMILES: *c1cccc2c1C(=O)N([C@H]1CCC(=O)NC1=O)C2=O]

In some embodiments, LBM is

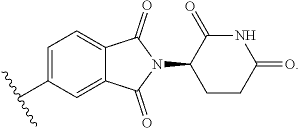 [SMILES: *c1ccc2c(c1)C(=O)N([C@@H]1CCC(=O)NC1=O)C2=O]

In some embodiments, LBM is

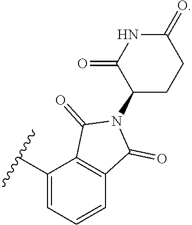 [SMILES: *c1cccc2c1C(=O)N([C@@H]1CCC(=O)NC1=O)C2=O]

In some embodiments, LBM is

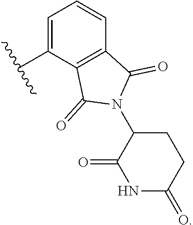 [SMILES: *c1cccc2c1C(=O)N(C1CCC(=O)NC1=O)C2=O]

In some embodiments, LBM is

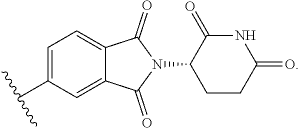 [SMILES: *c1ccc2c(c1)C(=O)N([C@H]1CCC(=O)NC1=O)C2=O]

In some embodiments, LBM is

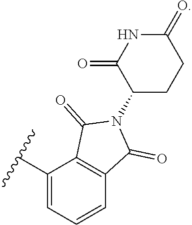 [SMILES: *c1cccc2c1C(=O)N([C@H]1CCC(=O)NC1=O)C2=O]

In certain embodiments, the present invention provides a compound of formula I, wherein LBM is a cereblon E3 ubiquitin ligase binding moiety thereby forming a compound of formula I-b:

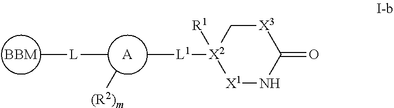

or a pharmaceutically acceptable salt thereof, wherein L and BBM are as defined above and described herein, and wherein:
X₁ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

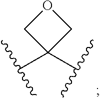 [SMILES: *C1(*)COC1]

X₂ is a carbon atom or silicon atom;
X₃ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂) OR, —P(O)(NR₂)₂, —Si(OH)₂R, —Si(OH)(R)₂, —Si(R)₃, or an optionally substituted C₁₋₄ aliphatic;
each R₂ is independently hydrogen, deuterium, —R₆, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R) OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

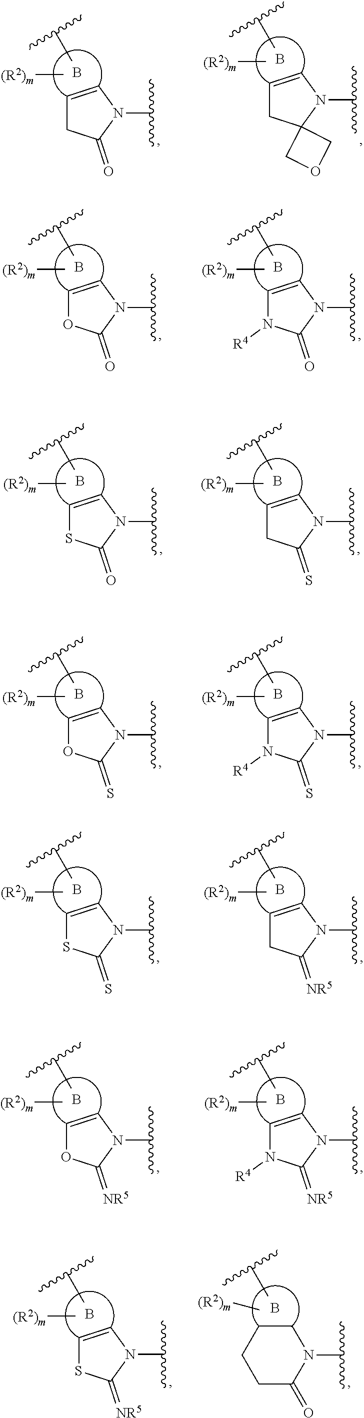

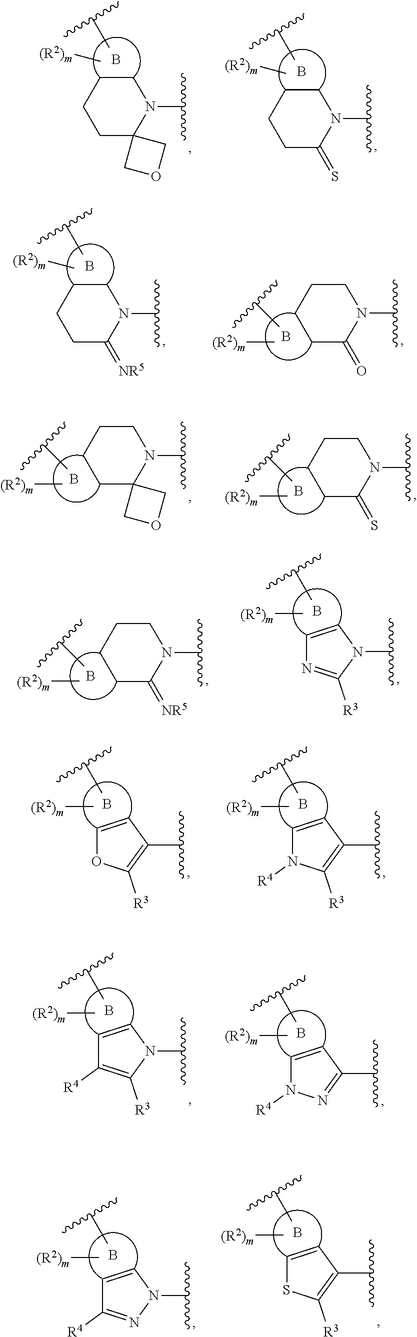

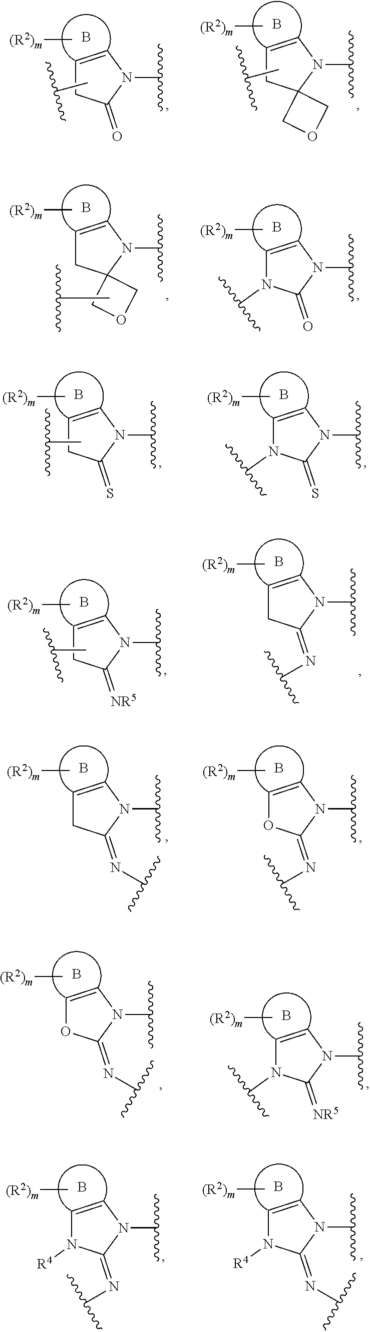

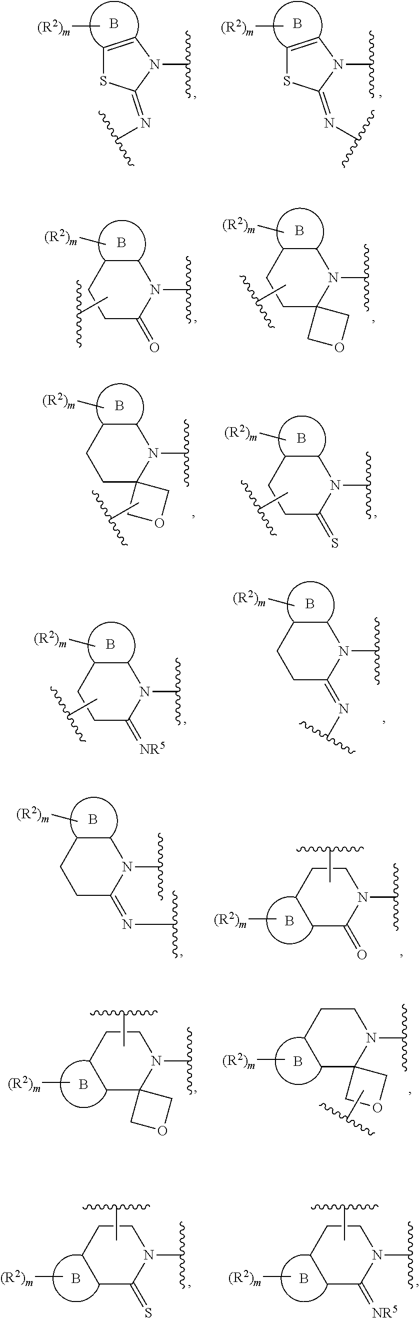

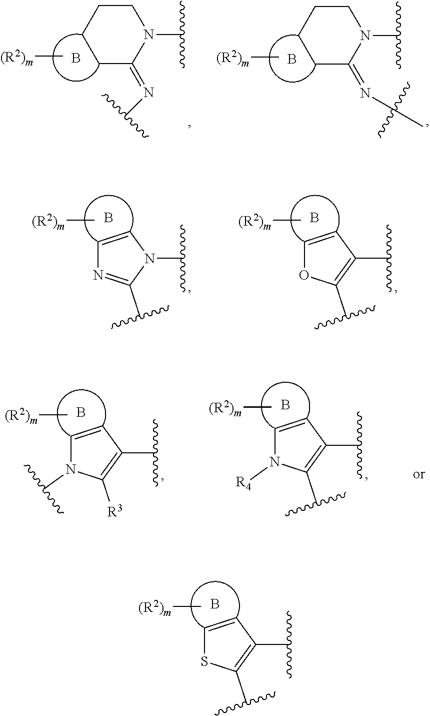

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R₃ is selected from hydrogen, halogen, —OR, —N(R)₂, or —SR;
each R⁴ is independently hydrogen, —R₆, halogen, —CN, —NO₂, —OR, —SR—NR₂, —S(O)₂R, —S(O)₂NR₂, —S(O)R, —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R;
R₅ is hydrogen, C₁₋₄ aliphatic, or —CN;
each R₆ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)—CH—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, a compound of formula I-b above is provided as a compound of formula I-b-1 or formula I-b-2:

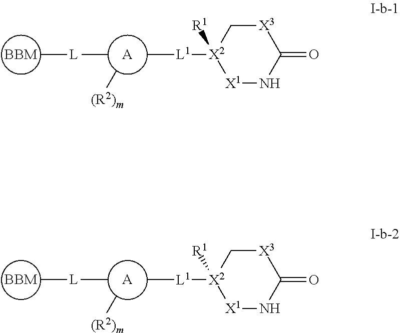

or a pharmaceutically acceptable salt thereof, wherein:
each of BBM, Ring A, L, L¹, R¹, R², X¹, X², X³, and m is as defined above.

In some embodiments, a compound of formula I-b above is provided as a compound of formula I-b-3:

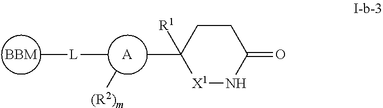

or a pharmaceutically acceptable salt thereof, wherein:
each of BBM, Ring A, L, R¹, R², X¹, and m is as defined above.

In certain embodiments, the present invention provides a compound of formula I, wherein LBM is a cereblon E3 ubiquitin ligase binding moiety thereby forming a compound of formula I-d:

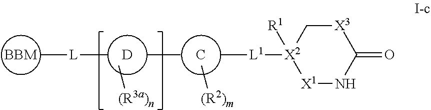

or a pharmaceutically acceptable salt thereof, wherein, L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

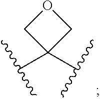 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —NR₂, —P(O)(OR)₂, —P(O)(NR₂) OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

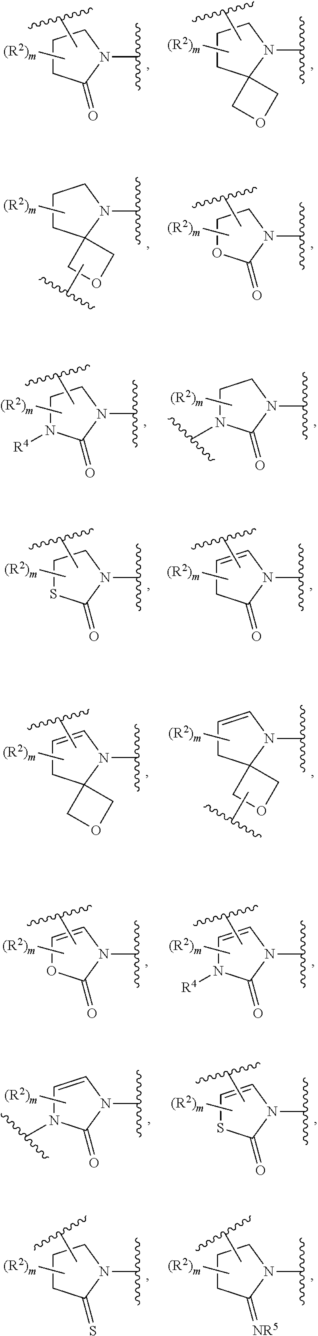

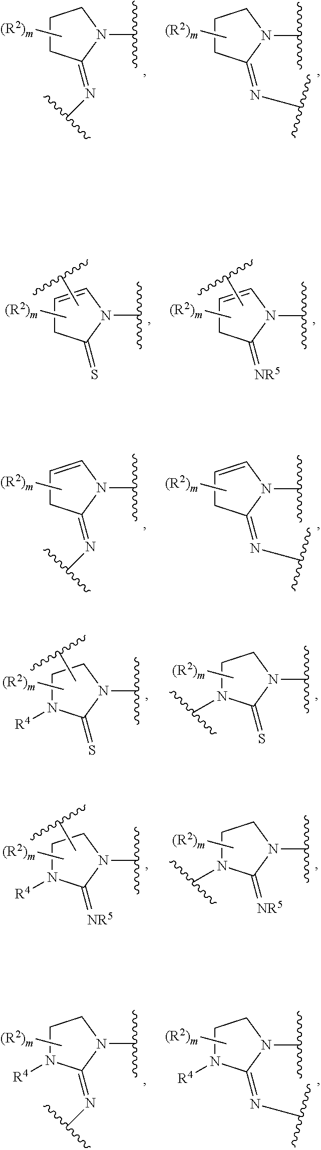

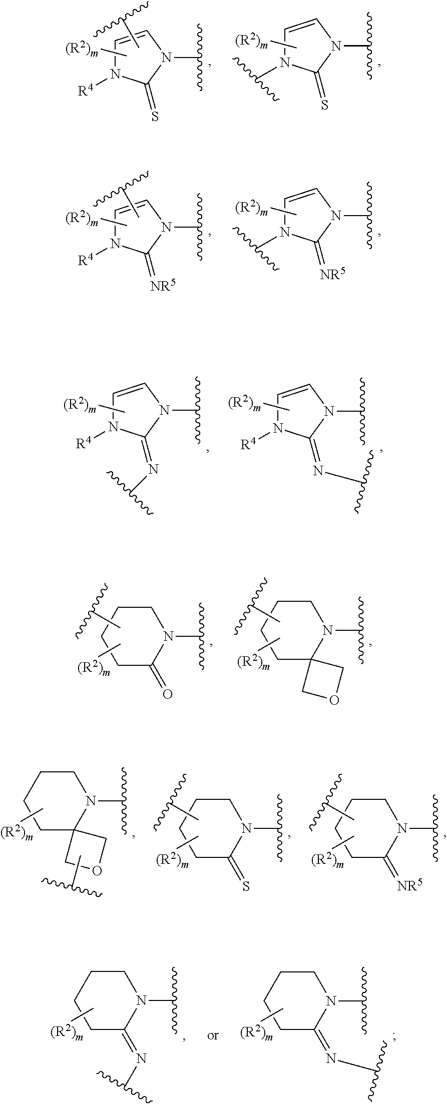

each of R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R) OR, —C(R)₂N(R)C(O)R, —C(R)₂N(R)C(O)N(R)₂, —OC(O)R, —OC(O)N(R)₂, —OP(O)R², —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R², —N(R)P(O)(OR)₂, —N(R)P(O)(OR)(NR₂), —N(R)P(O)(NR₂)₂, or —N(R)S(O)₂R;
Ring D is selected from a 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1, wherein when p is 0, the bond connecting Ring C and Ring D is connected to

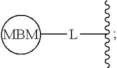

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-c above is provided as a compound of formula I-c-1 or formula I-c-2:

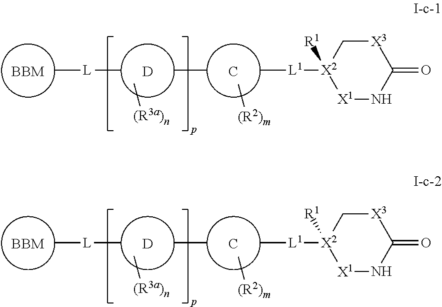

or a pharmaceutically acceptable salt thereof, wherein:
each of BBM, Ring C, Ring D, L, L¹, R¹, R², R^{3a}, X¹, X², X³, n, m, and p is as defined above.

In some embodiments, a compound of formula I-c above is provided as a compound of formula I-c-3:

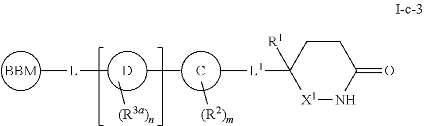

or a pharmaceutically acceptable salt thereof, wherein:
each of BBM, Ring C, Ring D, L, R¹, R², R^{3a}, X¹, n, m, and p is as defined above.

In certain embodiments, the present invention provides a compound of formula I, wherein LBM is a cereblon E3 ubiquitin ligase binding moiety thereby forming a compound of formula I-d:

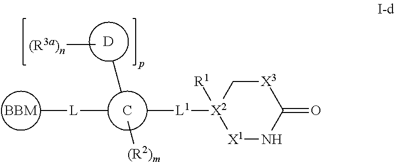

or a pharmaceutically acceptable salt thereof, wherein L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

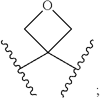 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

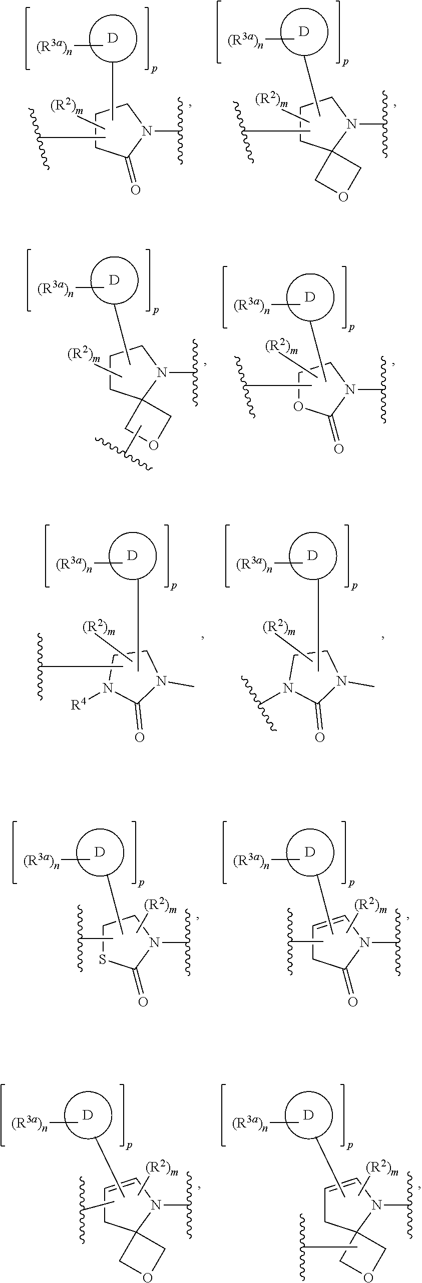

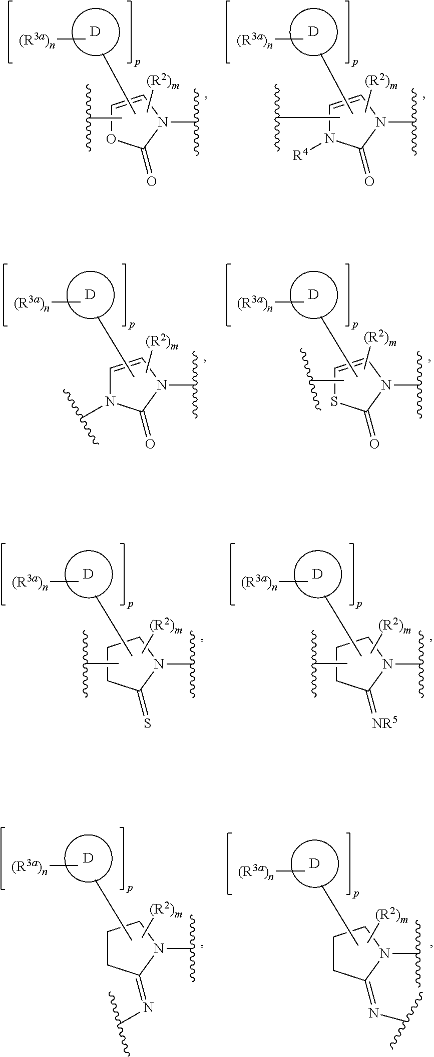

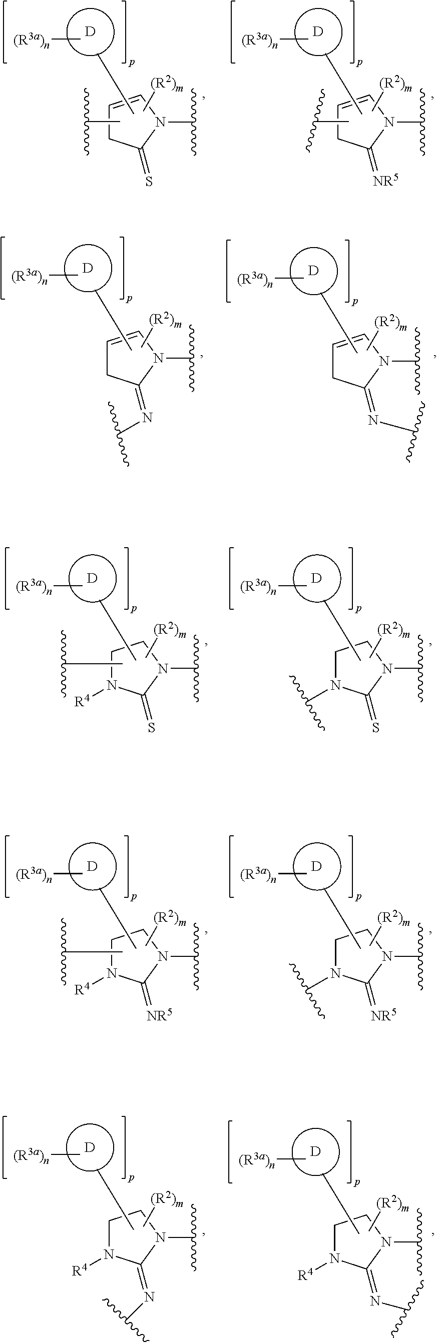

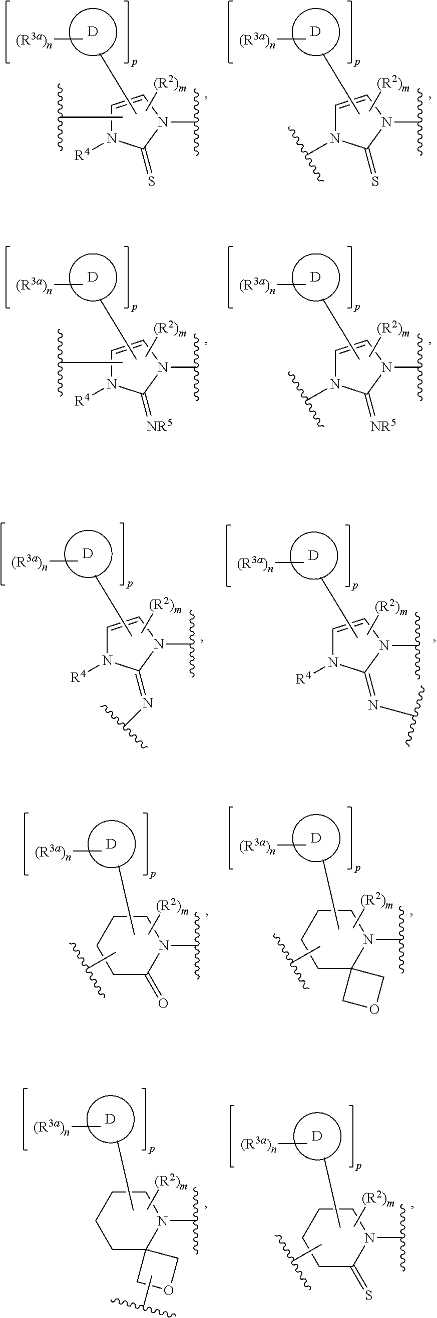

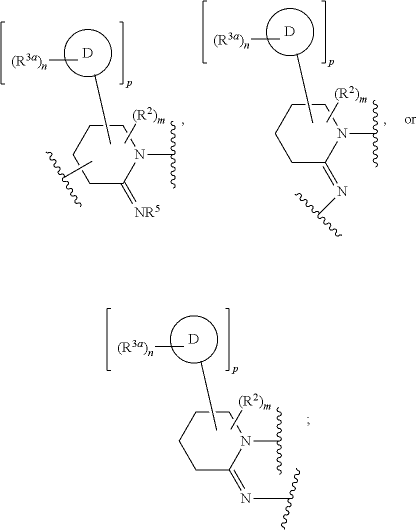

each or R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R) OR, —C(R)₂N(R)C(O)R, —C(R)₂N(R)C(O)N(R)₂, —OC(O)R, —OC(O)N(R)₂, —OP(O)R², —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R², —N(R)P(OX(OR)₂, —N(R)P(O)(OR)(NR₂), —N(R)P(O)(NR₂)₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR,

C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-d above is provided as a compound of formula I-d-1 or formula I-d-2:

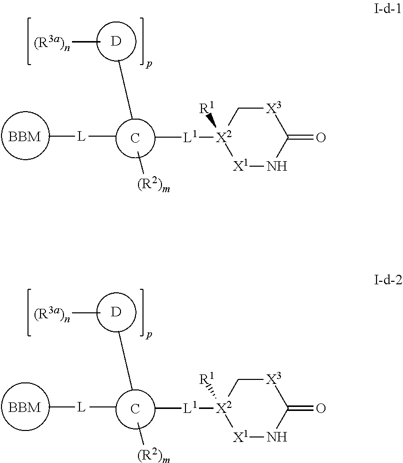

or a pharmaceutically acceptable salt thereof, wherein:
each of BBM, Ring C, Ring D, L, L¹, R¹, R², R^{3a}, X¹, X², X³, m, n, and p is as defined above.

In some embodiments, a compound of formula I-d above is provided as a compound of formula I-d-3:

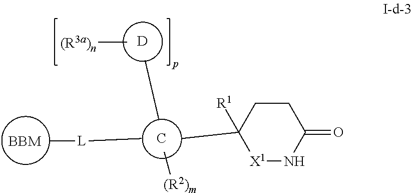

or a pharmaceutically acceptable salt thereof, wherein:
each of BBM, Ring C, Ring D, L, LR¹, R², R^{3a}, X¹, m, n, and p is as defined above.

In certain embodiments, the present invention provides a compound of formula I, wherein LBM is a cereblon E3 ubiquitin ligase binding moiety thereby forming a compound of formula I-e:

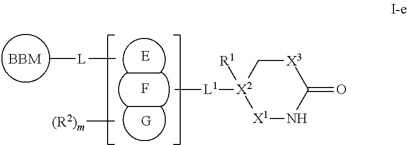

or a pharmaceutically acceptable salt thereof, wherein L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

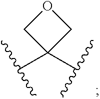 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —N(R)₂, —P(O)(OR)₂, —P(O)(NR₂) OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R) OR, —C(R) ¿N(R)C(O)R, —C(R)₂N(R)C(O)N(R)₂, —OC(O)R, —OC(O)N(R)₂, —OP(O)R², —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R², —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)—CH—; and
m is 0, 1, 2, 3, 4, 5, 6, 7, 8, 9, 10, 11, 12, 13, 14, 15, or 16.

Where a point of attachment of

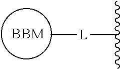

is depicted on Ring E, Ring F, or Ring G, it is intended, and one of ordinary skill in the art would appreciate, that the point of attachment of

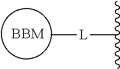

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

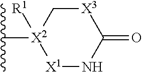

is depicted on Ring E, Ring F, or Ring G, it is intended, and one of ordinary skill in the art would appreciate, that the point of attachment of

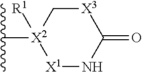

may be on any available carbon or nitrogen atom on Ring E, Ring F, or Ring G, including the carbon atom to which Ring E or Ring G are fused to Ring F.

In some embodiments, a compound of formula I-e above is provided as a compound of formula I-e-1 or formula I-e-2:

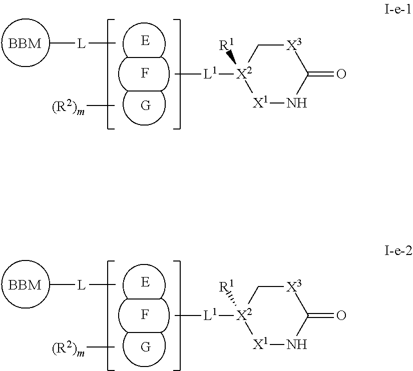

or a pharmaceutically acceptable salt thereof, wherein:
each of BBM, Ring E, Ring F, Ring G, L, L¹, R¹, R², X¹, X², X³, and m is as defined above.

In some embodiments, a compound of formula I-e above is provided as a compound of formula

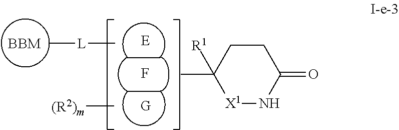

or a pharmaceutically acceptable salt thereof, wherein:
each of BBM, Ring E, Ring F, Ring G, L, R¹, R², X¹, and m is as defined above.

In certain embodiments, the present invention provides a compound of formula I, wherein LBM is an E3 ubiquitin ligase (cereblon) binding moiety thereby forming a compound of formula I-f:

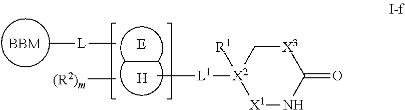

or a pharmaceutically acceptable salt thereof, wherein L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

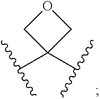 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —N(R)₂, —P(O)(OR)₂, —P(O)(NR₂) OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R) OR, —C(R)₂N(R)C(O)R, —C(R)₂N(R)C(O)N(R)₂, —OC(O)R, —OC(O)N(R)₂, —OP(O)R², —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R², —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring H is a fused ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)—CH—;
m is 0, 1, 2, 3, or 4.

Where a point of attachment of

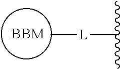

is depicted on Ring E or Ring H, it is intended, and one of ordinary skill in the art would appreciate, that the point of attachment of

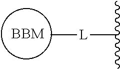

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

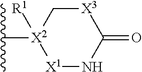

is depicted on Ring E and Ring H, it is intended, and one of ordinary skill in the art would appreciate, that the point of attachment of

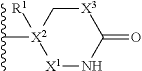

may be on any available carbon or nitrogen atom on Ring E or Ring H including the carbon atom to which Ring E and Ring H are fused.

In some embodiments, a compound of formula I-f above is provided as a compound of formula I-f-1 or formula I-f-2:

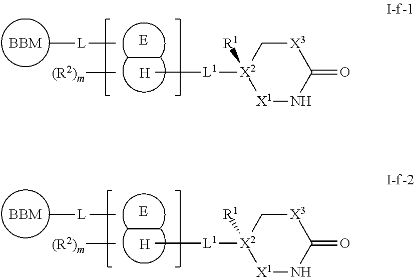

or a pharmaceutically acceptable salt thereof, wherein:
each of BBM, Ring E, Ring H, L, L¹, R¹, R², X¹, X², X³, and m is as defined above.

In some embodiments, a compound of formula I-f above is provided as a compound of formula I-f-3:

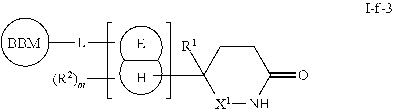

or a pharmaceutically acceptable salt thereof, wherein:
each of BBM, Ring E, Ring H, L, R¹, R², X¹, and m is as defined above.

In certain embodiments, the present invention provides a compound of formula I, wherein LBM is an E3 ubiquitin ligase (cereblon) binding moiety thereby forming a compound of formula I-g:

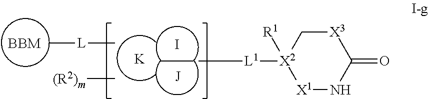

or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

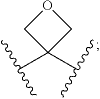 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —NR₂, —P(O)(OR)₂, —P(O)(NR₂) OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R) OR, —C(R)₂N(R)C(O)R, —C(R); N(R)C(O)N(R)₂, —OC(O)R, —OC(O)N(R)₂, —OP(O)R², —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R², —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring K is a fused ring selected from a 6-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and m is 0, 1, 2, 3, or 4.

Where a point of attachment of

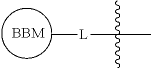

is depicted on Ring I, Ring J, and Ring K, it is intended, and one of ordinary skill in the art would appreciate, that the point of attachment of

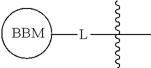

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

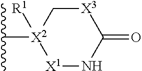

is depicted on Ring I, Ring J, and Ring K, it is intended, and one of ordinary skill in the art would appreciate, that the point of attachment of

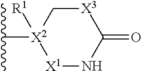

may be on any available carbon or nitrogen atom on Ring I, Ring J, or Ring K, including the carbon atom to which Ring I, Ring J, and Ring K are fused.

In some embodiments, a compound of formula I-g above is provided as a compound of formula I-g-1 or formula I-g-2:

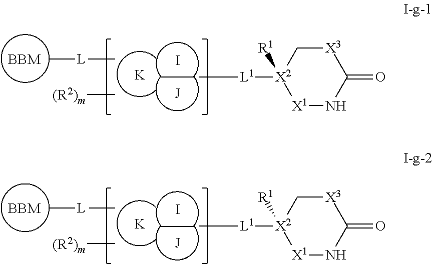

or a pharmaceutically acceptable salt thereof, wherein:
each of BBM, Ring I, Ring J, Ring K, L, L¹, R¹, R², X¹, X², X³, and m is as defined above.

In some embodiments, a compound of formula I-g above is provided as a compound of formula I-g-3:

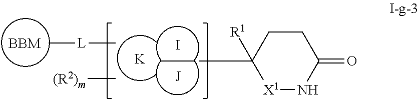

or a pharmaceutically acceptable salt thereof, wherein:
each of BBM, Ring I, Ring J, Ring K, L, R¹, R², X¹, and m is as defined above.

In certain embodiments, the present invention provides a compound of formula I, wherein LBM is an E3 ubiquitin ligase (cereblon) binding moiety thereby forming a compound of formula I-h-1 or I-h-2:

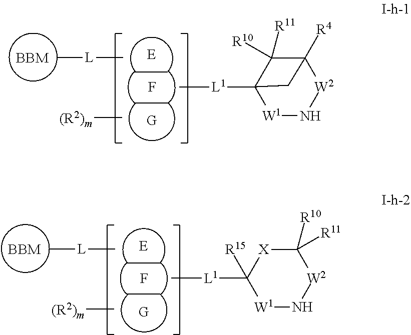

or a pharmaceutically acceptable salt thereof, wherein L and BBM are as defined above and described in embodiments herein, and wherein:
each R² is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R) OR, —C(R)₂N(R)C(O)R, —C(R)₂N(R)C(O)N(R)₂, —OC(O)R, —OC(O)N(R)₂, —OP(O)R², —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R², —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)—CH—;
m is 0, 1, 2, 3, 4, 5, 6, 7, 8, 9, 10, 11, 12, 13, 14, 15, or 16; and
R⁴, R¹⁰, R¹¹, R¹⁵, W¹, W², and X is as defined in WO 2019/099868, the entirety of each of which is herein incorporated by reference.

Where a point of attachment of

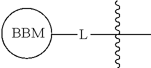

is depicted on Ring E, Ring F, or Ring G, it is intended, and one of ordinary skill in the art would appreciate, that the point of attachment of

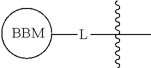

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of is

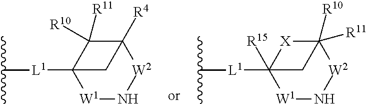

depicted on Ring E, Ring F, or Ring G, it is intended, and one of ordinary skill in the art would appreciate, that the point of attachment of

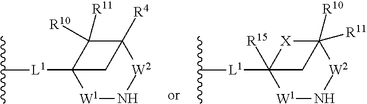

may be on any available carbon or nitrogen atom on Ring E, Ring F, or Ring G, including the carbon atom to which Ring E or Ring G are fused to Ring F.

As described above, in another aspect, the present invention provides a compound of formula I, wherein said compound is a compound of formula I-h-3:

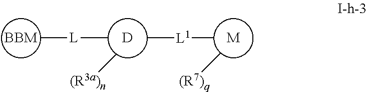

or a pharmaceutically acceptable salt thereof, wherein:
Ring M is selected from

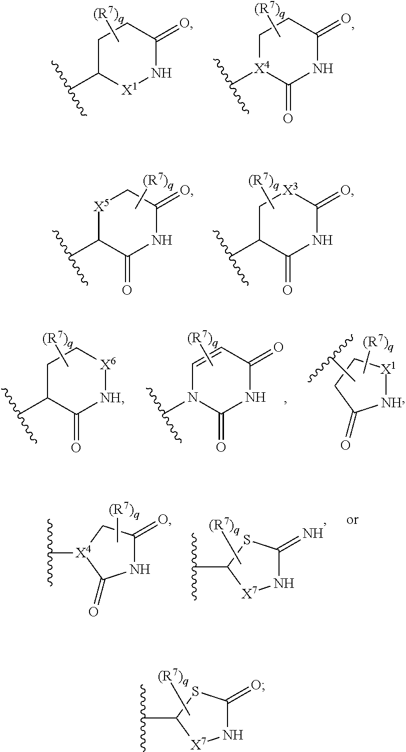

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

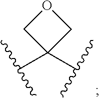 [SMILES: *C1(*)COC1]

each of X³ and X⁵ is independently a bivalent moiety selected from a covalent bond, —CR₂—, —NR—, —O—, —S—, or —SiR₂—;

X⁴ is a trivalent moiety selected from

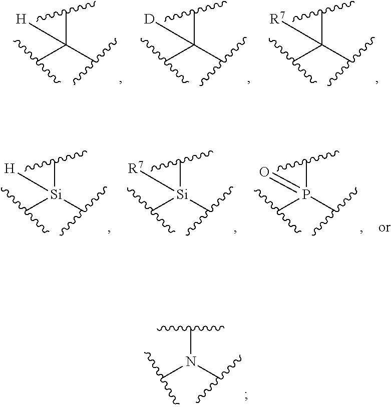

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R) OR, —C(R)₂N(R)C(O)R, —C(R); N(R)C(O)N(R)₂, —OC(O)R, —OC(O)N(R)₂, —OP(O)R², —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R², —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, deuterium, halogen, —CN, —OR, —SR, —S(O)R, —S(O)₂R, —NR₂, —P(O)(OR)₂, —P(O)(NR₂) OR, —P(O)(NR₂)₂, —Si(OH) R², —Si(OH)₂R, —SiR₃, or an optionally substituted C₁₋₄ aliphatic: or
   R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur;
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —(C)═CH—;
n is 0, 1, 2, 3, or 4; and
q is 0, 1, 2, 3, or 4.

As defined above and described herein, each of X¹, X⁶, and X⁷ is independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

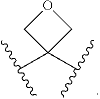 [SMILES: *C1(*)COC1]

In some embodiments, X¹, X⁶, and/or X⁷ is a covalent bond. In some embodiments, X¹, X⁶, and/or X⁷ is —CH₂—. In some embodiments, X¹, X⁶, and/or X⁷ is —CR₂—. In some embodiments, X¹, X⁶, and/or X⁷ is —C(O)—. In some embodiments, X¹, X⁶, and/or X⁷ is —C(S)—. In some embodiments, X¹, X⁶, and/or X⁷ is —CH(R)—. In some embodiments, X¹, X⁶, and/or X⁷ is —CH(CF₃)—. In some embodiments, X¹, X⁶, and/or X⁷ is —P(O)(OR)—. In some embodiments, X¹, X⁶, and/or X⁷ is —P(O)(R)—. In some embodiments, X¹, X⁶, and/or X⁷ is —P(O)NR₂—. In some embodiments, X¹, X⁶, and/or X⁷ is —S(O)—. In some embodiments, X¹, X⁶, and/or X⁷ is —S(O)₂—. In some embodiments, X¹, X⁶, and/or X⁷ is

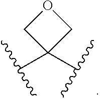 [SMILES: *C1(*)COC1]

In some embodiments, each of X¹, X⁶, and X⁷ are independently selected from those depicted in Table 1 below.

As defined above and described herein, X² is a carbon atom or silicon atom.

In some embodiments, X² is a carbon atom. In some embodiments, X² is a silicon atom.

In some embodiments, X² is selected from those depicted in Table 1, below.

As defined above and described herein, each of X³ and X⁵ is independently a bivalent moiety selected from —CH₂—, —CR₂—, —NR—, —CF₂—, —CHF—, —S—, —CH(R)—, —SiR₂—, or —O—.

In some embodiments, X³ and/or X⁵ is —CH₂—. In some embodiments, X³ and/or X⁵ is —CR₂—. In some embodiments, X³ and/or X⁵ is —NR—. In some embodiments, X³ and/or X⁵ is —CF₂—. In some embodiments, X³ and/or X⁵ is —CHF—. In some embodiments, X³ and/or X⁵ is —S—. In some embodiments, X³ and/or X⁵ is —CH(R)—. In some embodiments, X³ and/or X⁵ is —SiR₂—. In some embodiments, X³ and/or X⁵ is —O—.

In some embodiments, each of X³ and X⁵ is independently selected from those depicted in Table 1 below.

As defined above and described herein, X⁴ is a trivalent moiety selected from

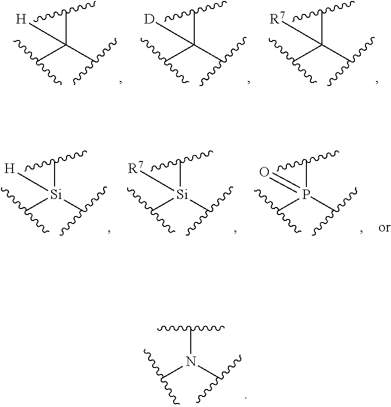

In some embodiments, X⁴ is

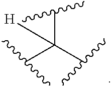 [SMILES: *C(*)*]

In some embodiments, X⁴ is

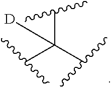 [SMILES: *C(*)(*)[2H]]

In some embodiments, X⁴ is

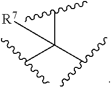

In some embodiments, X⁴ is

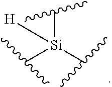 [SMILES: *[SiH](*)*]

In some embodiments, X⁴ is

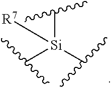

In some embodiments, X⁴ is

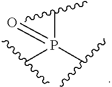 [SMILES: *P(*)(*)=O]

In some embodiments, X⁴ is

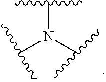 [SMILES: *N(*)*]

In some embodiments, X⁴ is selected from those depicted in Table 1 below.

As defined above and described herein, R¹ is hydrogen, deuterium, halogen, —CN, —OR, —SR, —S(O)R, —S(O)₂R, —NR₂, —P(O)(OR)₂, —P(O)(NR₂) OR, —P(O)(NR₂)₂, —Si(OH)₂R, —Si(OH)(R)₂, —Si(R)₃, an optionally substituted C₁₋₄ aliphatic, or R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R is —S(O)R. In some embodiments, R¹ is —S(O)₂R. In some embodiments, R¹ is —NR₂. In some embodiments, R¹ is —P(O)(OR)₂. In some embodiments, R¹ is —P(O)(NR₂) OR. In some embodiments, R¹ is —P(O)(NR₂)₂. In some embodiments, R¹ is —Si(OH)₂R. In some embodiments, R¹ is —Si(OH)(R)₂. In some embodiments, R¹ is —Si(R)₃. In some embodiments, R¹ is an optionally substituted C₁₋₄ aliphatic. In some embodiments, R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is selected from those depicted in Table 1, below.

As defined above and described herein, each R is independently hydrogen, deuterium, or an optionally substituted group selected from C₁₋₆ aliphatic, phenyl, a 4-7 membered saturated or partially unsaturated heterocyclic having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur, or two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is hydrogen. In some embodiments, R is deuterium. In some embodiments, R is optionally substituted C₁₋₆ aliphatic. In some embodiments, R is optionally substituted phenyl. In some embodiments, R is optionally substituted 4-7 membered saturated or partially unsaturated heterocyclic having 1-3 heteroatoms independently selected from boron, nitrogen, oxygen, silicon, and sulfur. In some embodiments, R is optionally substituted 5-6 membered heteroaryl ring having 1-4 heteroatoms independently selected from boron, nitrogen, oxygen, silicon, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is selected from those depicted in Table 1, below.

As defined above and described herein, each of R² and R^{3a} is independently hydrogen, deuterium, —R⁶, halogen, n, —CN, —NO₂, —OR, —Si(OH)₂R, —Si(OH) R², —SR, —NR₂, —SiR₃, —S(O)₂R, —S(O); NR₂, —S(O)R, —C(O)R, —C(O)OR, —C(O)NR₂, —C(O)N(R) OR, —C(R)₂N(R)C(O)R, —C(R) ¿N(R)C(O)NR₂, —OC(O)R, —OC(O)NR₂, —OP(O)R², —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R², —N(R)P(O)(OR)₂, —N(R)P(O)(OR)NR₂, —N(R)P(O)(NR₂)₂, or —N(R)S(O)₂R.

In some embodiments, R² and/or R^{3a} is hydrogen. In some embodiments, R² and/or R^{3a} is deuterium. In some embodiments, R² and/or R^{3a} is —R⁶. In some embodiments, R² and/or R^{3a} is halogen. In some embodiments, R² and/or R^{3a} is —CN. In some embodiments, R² and/or R^{3a} is —NO₂. In some embodiments, R² and/or R^{3a} is —OR. In some embodiments, R² and/or R^{3a} is —Si(OH)₂R. In some embodiments, R² and/or R^{3a} is —Si(OH) R². In some embodiments, R² and/or R^{3a} is —SR. In some embodiments, R² and/or R^{3a} is —NR₂. In some embodiments, R² and/or R^{3a} is —SiR₃. In some embodiments. R² and/or R^{3a} is —S(O)₂R. In some embodiments, R² and/or R^{3a} is —S(O)₂NR₂. In some embodiments, R² and/or R^{3a} is —S(O)R. In some embodiments, R² and/or R^{3a} is —C(O)R. In some embodiments, R² and/or R^{3a} is —C(O)OR. In some embodiments, R² and/or R^{3a} is —C(O)NR₂. In some embodiments, R² and/or R^{3a} is —C(O)N(R) OR. In some embodiments, R² and/or R^{3a} is —C(R)₂N(R)C(O)R. In some embodiments, R² and/or R^{3a} is —C(R)₂N(R)C(O)NR₂. In some embodiments, R² and/or R^{3a} is —OC(O)R. In some embodiments, R² and/or R^{3a} is —OC(O)NR₂. In some embodiments, R² and/or R^{3a} is —OP(O)R². In some embodiments, R² and/or R^{3a} is —OP(O)(OR)₂. In some embodiments, R² and/or R^{3a} is —OP(O)(OR)NR₂. In some embodiments, R² and/or R^{3a} is —OP(O)(NR₂)₂—. In some embodiments, R² and/or R^{3a} is —N(R)C(O)OR. In some embodiments, R² and R^{3a} is independently —N(R)C(O)R. In some embodiments. R² and/or R^{3a} is —N(R)C(O)NR₂. In some embodiments, R² and/or R^{3a} is —NP(O)R². In some embodiments. R² and/or R^{3a} is —N(R)P(O)(OR)₂. In some embodiments, R² and/or R^{3a} is —N(R)P(O)(OR)NR₂. In some embodiments, R² and/or R^{3a} is —N(R)P(O)(NR₂)₂. In some embodiments, R² and/or R^{3a} is —N(R)S(O)₂R.

In some embodiments, R² and R^{3a} is independently —OH. In some embodiments, R² and R^{3a} is independently —NH₂. In some embodiments, R² and R^{3a} is independently —CH₂NH₂. In some embodiments. R² and R^{a} is independently —CH₂NHCOMe. In some embodiments, R² and R^{3a} is independently —CH₂NHCONHMe. In some embodiments, R² and R^{3a} is independently —NHCOMe. In some embodiments. R² and R^{3a} is independently —NHCONHEt. In some embodiments, R² and R^{3a} is independently —SiMe₃. In some embodiments, R² and R^{3a} is independently —SiMe₂OH. In some embodiments, R² and R^{3a} is independently —SiMe(OH)₂. In some embodiments R² and/or R^{3a} is

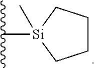 [SMILES: *[Si]1(C)CCCC1]

In some embodiments, R² and/or R^{3a} is Br. In some embodiments, R² and/or R^{3a} is C₁. In some embodiments, R² and/or R^{3a} is F. In some embodiments, R² and/or R^{3a} is Me. In some embodiments, R² and/or R^{3a} is —NHMe. In some embodiments, R² and/or R^{3a} is —NMe₂. In some embodiments, R² and/or R^{3a} is —NHCO₂Et. In some embodiments, R² and/or R^{3a} is —CN. In some embodiments, R² and/or R^{3a} is —CH₂Ph. In some embodiments, R² and/or R^{3a} is —NHCO₂/Bu. In some embodiments, R² and/or R^{3a} is —CO₂/Bu. In some embodiments, R² and/or R^{3a} is —OMe. In some embodiments, R² and/or R^{3a} is —CF₃.

In some embodiments, R² and R^{3a} are selected from those depicted in Table 1, below.

As defined above and described herein. R³ is hydrogen, deuterium, halogen, —CN, —NO₂, —OR. —NR₂, —SR, —S(O)₂R, —S(O)₂NR₂, —S(O)R, —C(O)R, —C(O)OR, —C(O)NR₂, —C(O)NR(OR), —OC(O)R, —OC(O)NR₂, —OP(O)(OR)₂, —OP(O)(NR₂)₂, —OP(O)(OR)NR₂, —N(R)C(O)R, N(R)C(O)OR, —N(R)C(O)NR₂, —N(R)S(O)₂R, —N(R)S(O)₂NR₂, —N(R)P(O)(OR)₂, —N(R)P(O)(OR)NR₂, —P(O)(OR)₂, —P(O)(NR₂) OR, —P(O)(NR₂)₂, —Si(OH)₂R, —Si(OH)(R)₂, or —Si(R)₃.

In some embodiments, R³ is hydrogen. In some embodiments, R³ is deuterium. In some embodiments, R³ is halogen. In some embodiments, R³ is —CN. In some embodiments, R³ is —NO₂. In some embodiments, R³ is —OR. In some embodiments, R³ is —NR₂. In some embodiments, R³ is —SR. In some embodiments, R³ is —S(O)₂R. In some embodiments, R³ is —S(O)₂NR₂. In some embodiments, R³ is —S(O)R. In some embodiments, R³ is —C(O)R. In some embodiments, R³ is —C(O)OR. In some embodiments. R³ is —C(O)NR₂. In some embodiments, R³ is —C(O)NR(OR). In some embodiments, R³ is —OC(O)R. In some embodiments, R³ is —OC(O)NR₂. In some embodiments, R³ is —OP(O)(OR)₂. In some embodiments. R³ is —OP(O)(NR₂)₂. In some embodiments, R³ is —OP(O)(OR)NR₂. In some embodiments, R³ is —N(R)C(O)R. In some embodiments, R³ is —N(R)C(O)OR. In some embodiments, R³ is —N(R)C(O)NR₂. In some embodiments, R³ is —N(R)S(O)₂R. In some embodiments, R³ is —N(R)S(O)₂NR₂. In some embodiments, R³ is —N(R)P(O)(OR)₂. In some embodiments, R³ is —N(R)P(O)(OR)NR₂. In some embodiments, R³ is —P(O)(OR)₂. In some embodiments, R³ is —P(O)(NR₂) OR. In some embodiments, R³ is —P(O)(NR₂)₂. In some embodiments, R³ is —Si(OH)₂R. In some embodiments, R³ is —Si(OH)(R)₂. In some embodiments, R³ is —Si(R)₃.

In some embodiments, R³ is methyl. In some embodiments, R³ is —OCH₃. In some embodiments, R³ is chloro.

In some embodiments, R³ is selected from those depicted in Table 1, below.

As defined above and described herein, each R⁴ is independently hydrogen, deuterium, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, —N(R)S(O)₂R, —P(O)(OR)₂, —P(O)(NR₂) OR, or —P(O)(NR₂)₂.

In some embodiments, R⁴ is hydrogen. In some embodiments, R⁴ is —R⁶. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R) OR. In some embodiments, R⁴ is —OC(O)R. In some embodiments, R⁴ is —OC(O)NR₂. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)S(O)₂R. In some embodiments, R⁴ is —P(O)(OR)₂. In some embodiments, R⁴ is —P(O)(NR₂) OR. In some embodiments, R⁴ is —P(O)(NR₂)₂.

In some embodiments, R⁴ is methyl. In some embodiments, R⁴ is ethyl. In some embodiments, R⁴ is cyclopropyl.

In some embodiments, R⁴ is selected from those depicted in Table 1, below.

As defined above and described herein, R⁵ is hydrogen, deuterium, an optionally substitute C₁₋₄ aliphatic, or —CN.

In some embodiments, R⁵ is hydrogen. In some embodiments, R⁵ is deuterium. In some embodiments, R⁵ is an optionally substituted C₁₋₄ aliphatic. In some embodiments, R⁵ is —CN.

In some embodiments, R⁵ is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur.

In some embodiments, R⁶ is an optionally substituted C₁₋₆ aliphatic. In some embodiments, R⁶ is an optionally substituted phenyl. In some embodiments, R⁶ is an optionally substituted 4-7 membered saturated or partially unsaturated heterocyclic ring having 1-3 heteroatoms independently selected from boron, nitrogen, oxygen, silicon, and sulfur. In some embodiments, R⁶ is an optionally substituted 5-6 membered heteroaryl ring having 1-4 heteroatoms independently selected from boron, nitrogen, oxygen, silicon, and sulfur.

In some embodiments, R⁶ is selected from those depicted in Table 1, below.

As defined generally above, each R⁷ is independently hydrogen, deuterium, halogen, —CN, —OR, —SR, —S(O)R, —S(O)₂R, —N(R)₂, —P(O)(R)₂, —P(O)(OR)₂, —P(O)(NR₂) OR, —P(O)(NR₂)₂, —Si(OH) R², —Si(OH)₂R, —SiR₃, or an optionally substituted C₁₋₄ aliphatic, or R¹ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur, or two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur, or two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is hydrogen. In some embodiments, R⁷ is deuterium. In some embodiments, R⁷ is halogen. In some embodiments, R⁷ is —CN. In some embodiments, R⁷ is —OR. In some embodiments, R⁷ is —SR. In some embodiments, R⁷ is —S(O)R. In some embodiments, R⁷ is —S(O)₂R. In some embodiments, R⁷ is —NR₂. In some embodiments, R⁷ is —Si(R)₂. In some embodiments, R⁷ is —P(O)(R)₂. In some embodiments, R⁷ is —P(O)(OR)₂. In some embodiments, R⁷ is —P(O)(NR₂) OR. In some embodiments, R⁷ is —P(O)(NR₂)₂. In some embodiments, R⁷ is —Si(OH) R². In some embodiments, R⁷ is —Si(OH)₂R. In some embodiments, R⁷ is an optionally substituted C₁₋₄ aliphatic. In some embodiments, R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur. In some embodiments, two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur. In some embodiments, two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur. In some embodiments, two R⁷ groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is selected from hydrogen, halogen, —CN, —OR, —NR₂, or C₁₋₄ alkyl. In some embodiments, R⁷ is selected from hydrogen, halogen, —CN, or C₁₋₄ alkyl. In some embodiments. R⁷ is fluoro. In some embodiments, two R⁷ groups on the same carbon are optionally taken together with their intervening atoms to form a 3- or 4-membered spiro fused ring.

In some embodiments, R⁷ is selected from those depicted in Table 1 below.

As defined above and described herein, Ring A is a bi- or tricyclic ring selected from

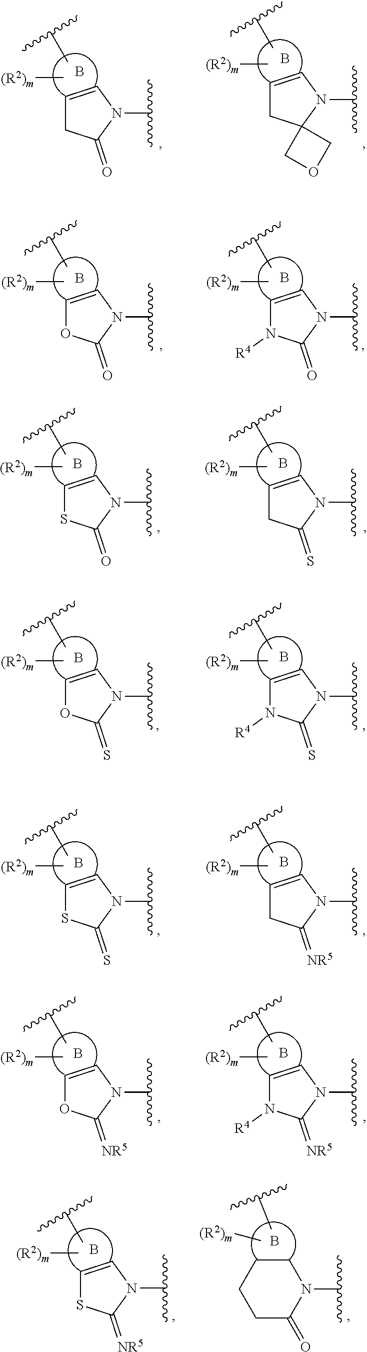

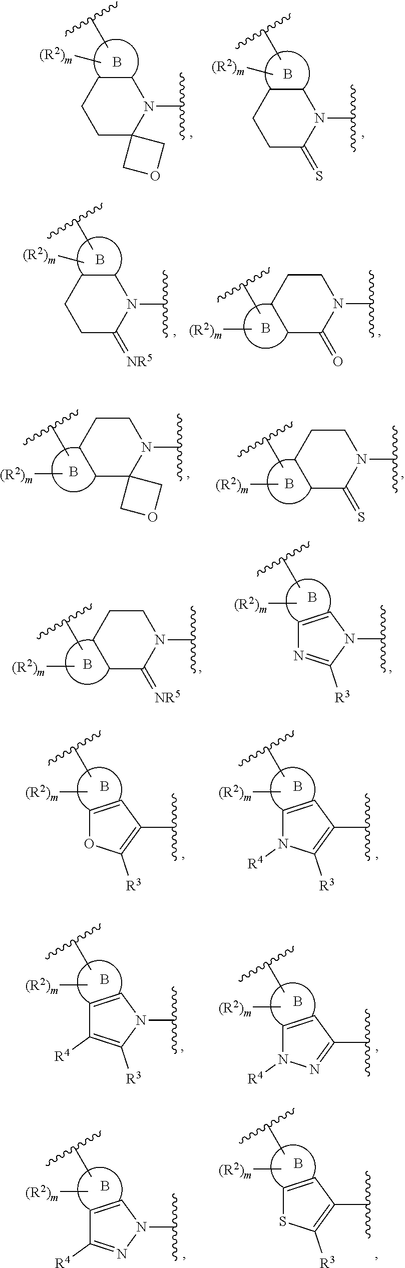

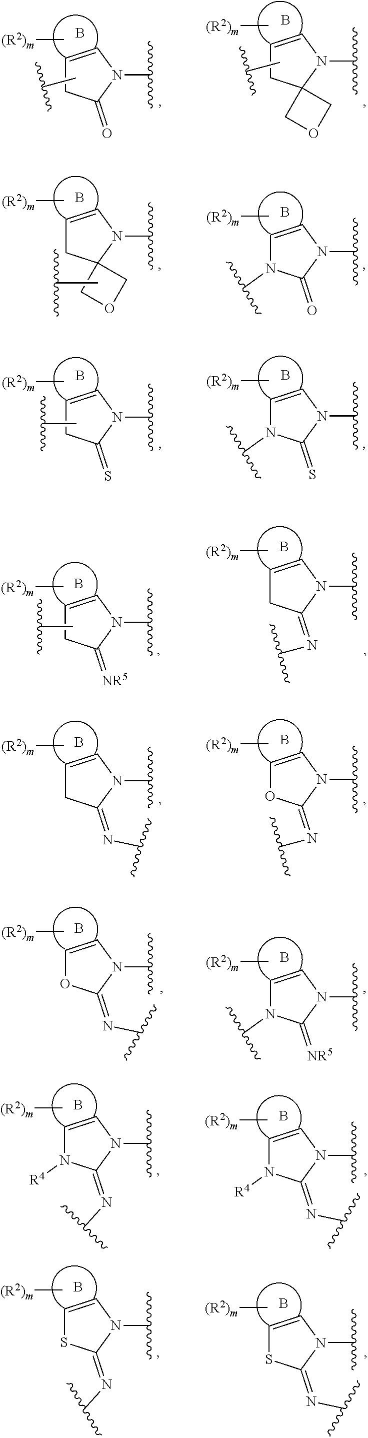

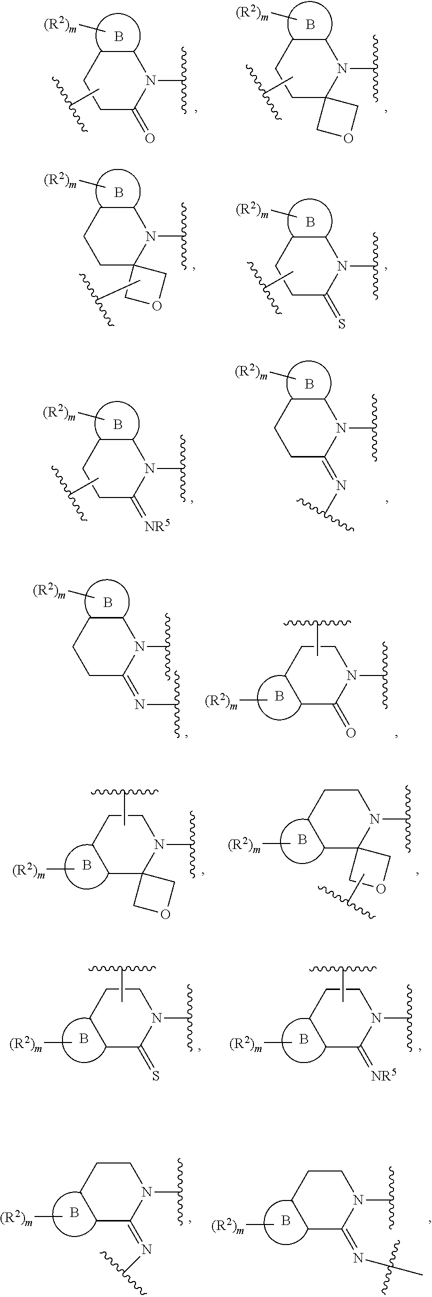

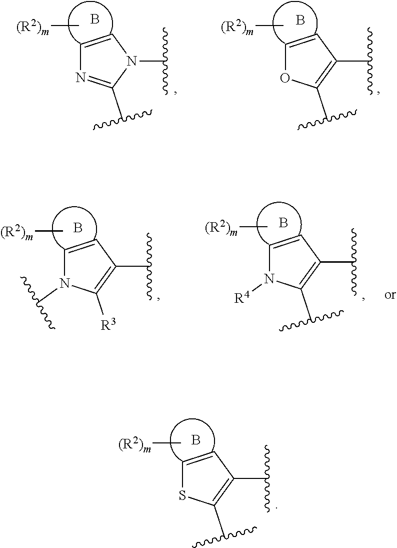

In some embodiments, Ring A is

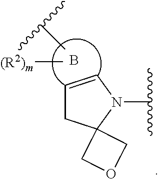

In some embodiments, Ring A is

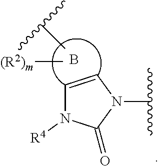

In some embodiments, Ring A is

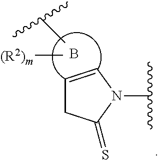

In some embodiments, Ring A is

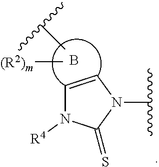

In some embodiments, Ring A is

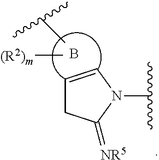

In some embodiments, Ring A is

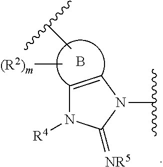

In some embodiments, Ring A is

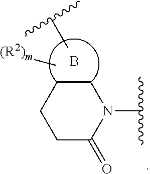

In some embodiments, Ring A is

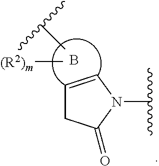

In some embodiments, Ring A is

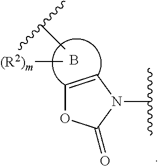

In some embodiments, Ring A is

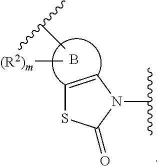

In some embodiments, Ring A is

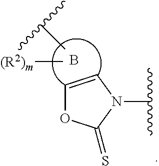

In some embodiments, Ring A is

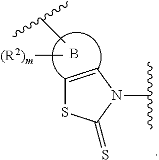

In some embodiments, Ring A is

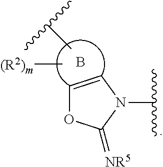

In some embodiments, Ring A is

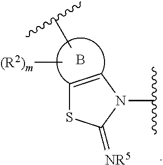

In some embodiments, Ring A is

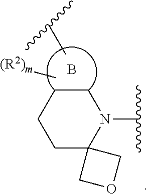

In some embodiments, Ring A is

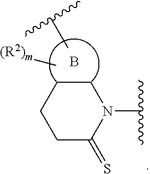

In some embodiments, Ring A is

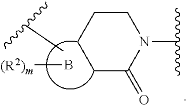

In some embodiments, Ring A is

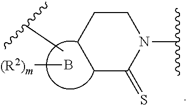

In some embodiments, Ring A is

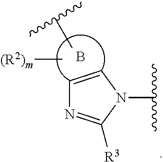

In some embodiments, Ring A is

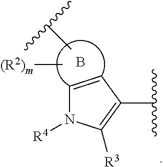

In some embodiments, Ring A is

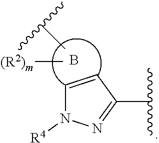

In some embodiments, Ring A is

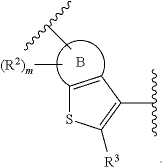

In some embodiments, Ring A is

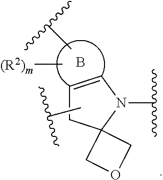

In some embodiments, Ring A is

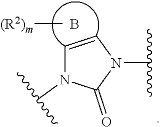

In some embodiments, Ring A is

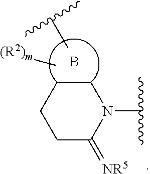

In some embodiments, Ring A is

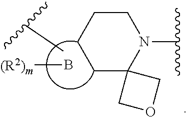

In some embodiments, Ring A is

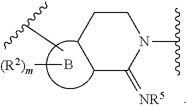

In some embodiments, Ring A is

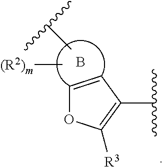

In some embodiments, Ring A is

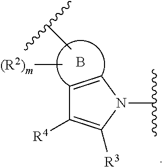

In some embodiments, Ring A is

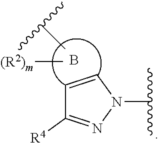

In some embodiments, Ring A is

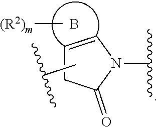

In some embodiments, Ring A is

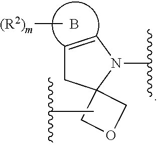

In some embodiments, Ring A is

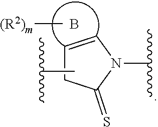

In some embodiments, Ring A is

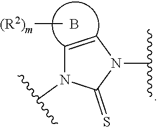

In some embodiments, Ring A is

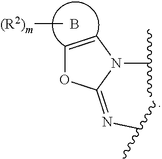

In some embodiments, Ring A is

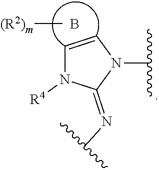

In some embodiments, Ring A is

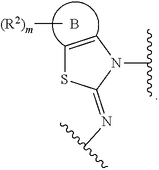

In some embodiments, Ring A is

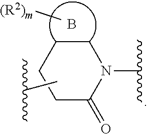

In some embodiments, Ring A is

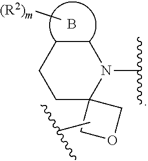

In some embodiments, Ring A is

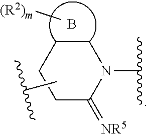

In some embodiments, Ring A is

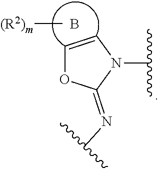

In some embodiments, Ring A is

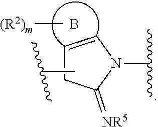

In some embodiments, Ring A is

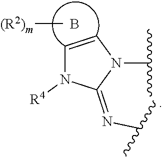

In some embodiments, Ring A is

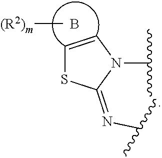

In some embodiments, Ring A is

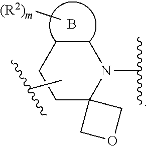

In some embodiments, Ring A is

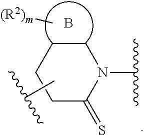

In some embodiments, Ring A is

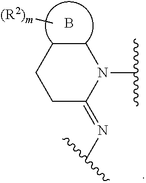

In some embodiments, Ring A is

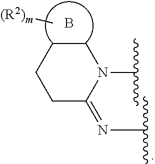

In some embodiments, Ring A is

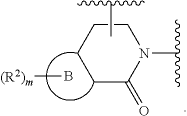

In some embodiments, Ring A is

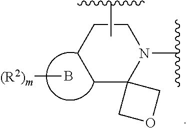

In some embodiments, Ring A is

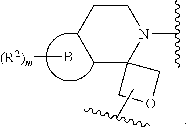

In some embodiments, Ring A is

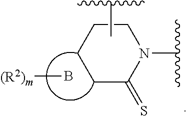

In some embodiments, Ring A is

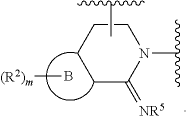

In some embodiments, Ring A is

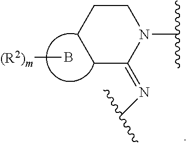

In some embodiments, Ring A is

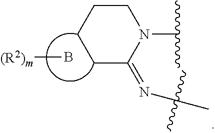

In some embodiments, Ring A is

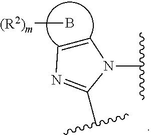

In some embodiments, Ring A is

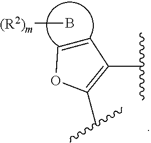

In some embodiments, Ring A is

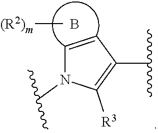

In some embodiments, Ring A is

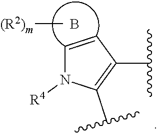

In some embodiments, Ring A is

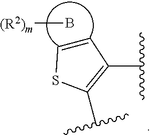

In some embodiments, Ring A is selected from those depicted in Table 1, below.

As defined above and described herein, Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring B is a fused 6-membered aryl. In some embodiments, Ring B is a fused 6-membered heteroaryl containing 1-4 heteroatoms independently selected from nitrogen, oxygen, and sulfur. In some embodiments, Ring B is a fused 5 to 7-membered saturated or partially unsaturated carbocyclyl. In some embodiments, Ring B is fused 5 to 7-membered saturated or partially saturated heterocyclyl with 1-3 heteroatoms independently selected from boron, nitrogen, oxygen, silicon, and sulfur. In some embodiments, Ring B is fused 5-membered heteroaryl with 1-4 heteroatoms independently selected from boron, nitrogen, oxygen, silicon, and sulfur.

In some embodiments, Ring B is

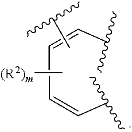

In some embodiments, Ring B is

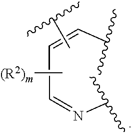

In some embodiments, Ring B is

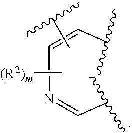

In some embodiments, Ring B is selected from those depicted in Table 1, below.

In some embodiments, Ring A and Ring B is

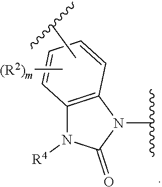

In some embodiments, Ring A and Ring B is

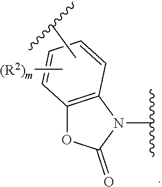

In some embodiments, Ring A and Ring B is

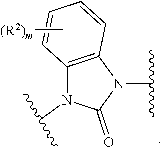

In some embodiments, Ring A and Ring B is

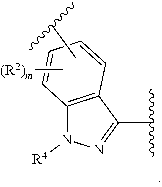

As defined above and described herein, Ring C is a mono- or bicyclic ring selected from

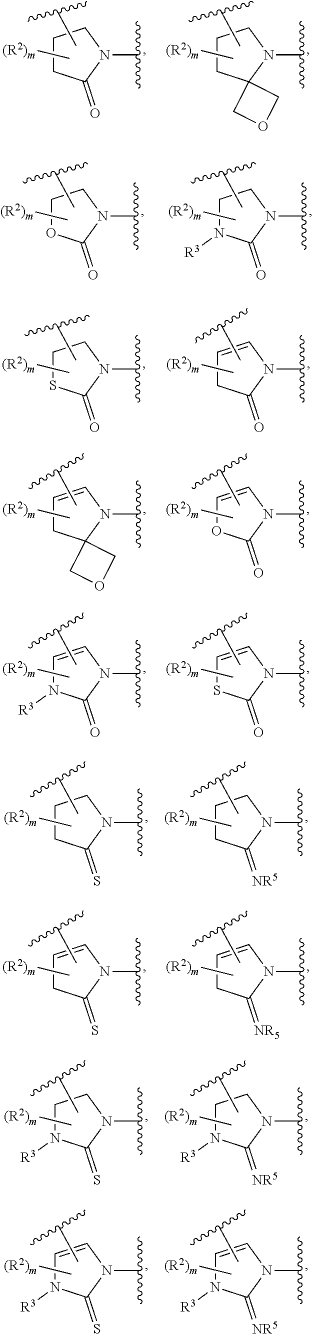

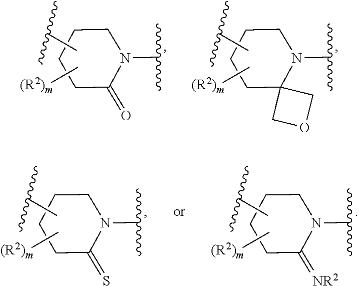

In some embodiments, Ring C is

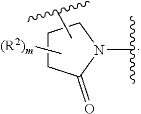

In some embodiments, Ring C is

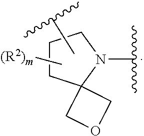

In some embodiments, Ring C is

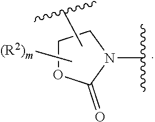

In some embodiments, Ring C is

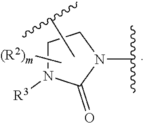

In some embodiments, Ring C is

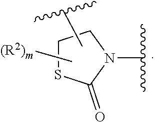

In some embodiments, Ring C is

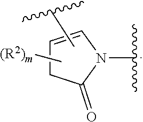

In some embodiments, Ring C is

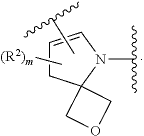

In some embodiments, Ring C is

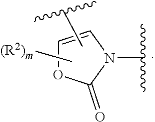

In some embodiments, Ring C is

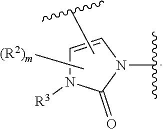

In some embodiments, Ring C is

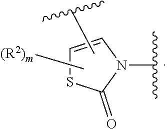

In some embodiments, Ring C is

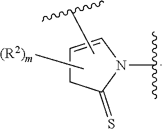

In some embodiments, Ring C is

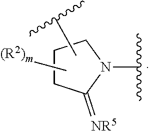

In some embodiments, Ring C is

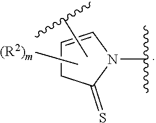

In some embodiments, Ring C is

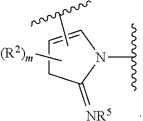

In some embodiments, Ring C is

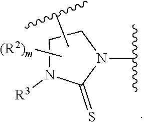

In some embodiments, Ring C is

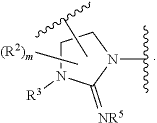

In some embodiments, Ring C is

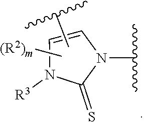

In some embodiments, Ring C is

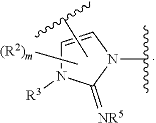

In some embodiments, Ring C is

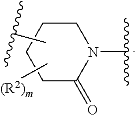

In some embodiments, Ring C is

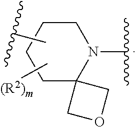

In some embodiments, Ring C is

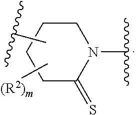

In some embodiments, Ring C is

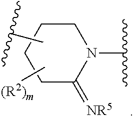

In some embodiments, Ring C is

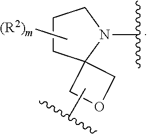

In some embodiments, Ring C is

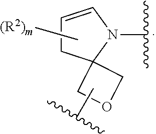

In some embodiments, Ring C is

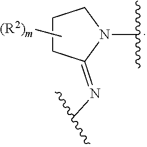

In some embodiments, Ring C is

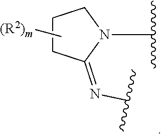

In some embodiments, Ring C is

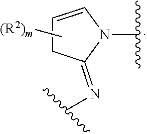

In some embodiments, Ring C is

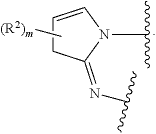

In some embodiments, Ring C is

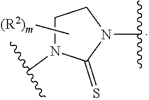

In some embodiments, Ring C is

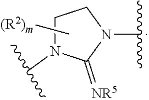

In some embodiments, Ring C is

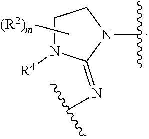

In some embodiments, Ring C is

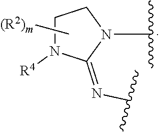

In some embodiments, Ring C is

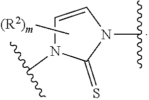

In some embodiments, Ring C is

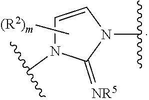

In some embodiments, Ring C is

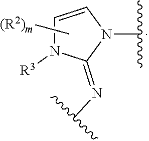

In some embodiments, Ring C is

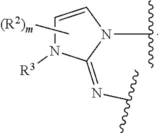

In some embodiments, Ring C is

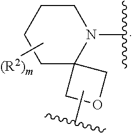

In some embodiments, Ring C is

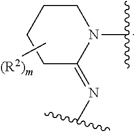

In some embodiments, Ring C is

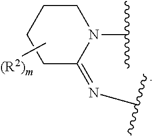

In some embodiments, Ring C is a mono- or bicyclic ring selected from

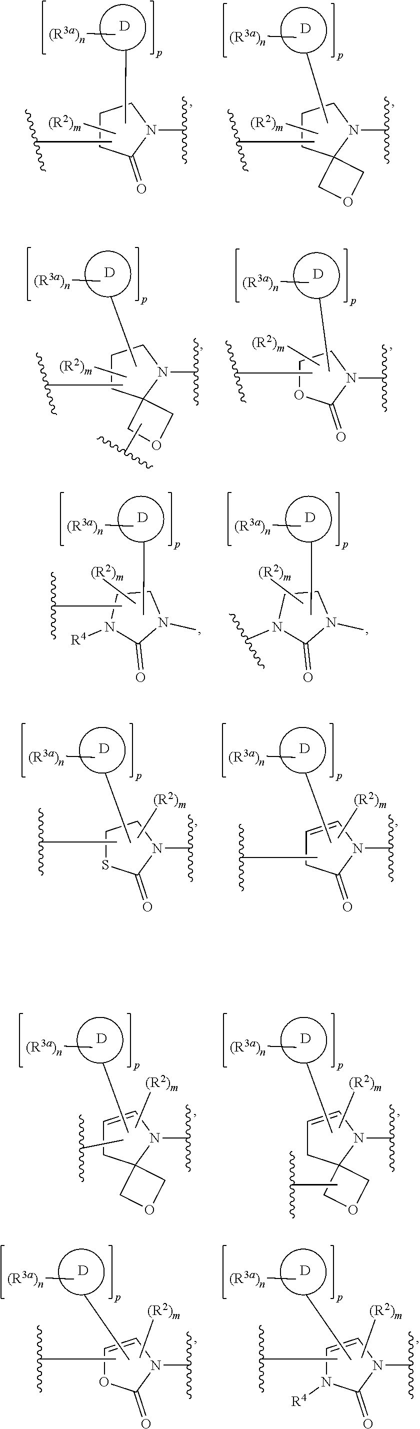

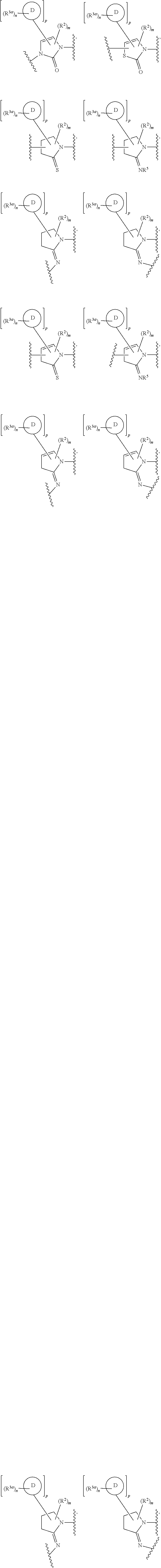

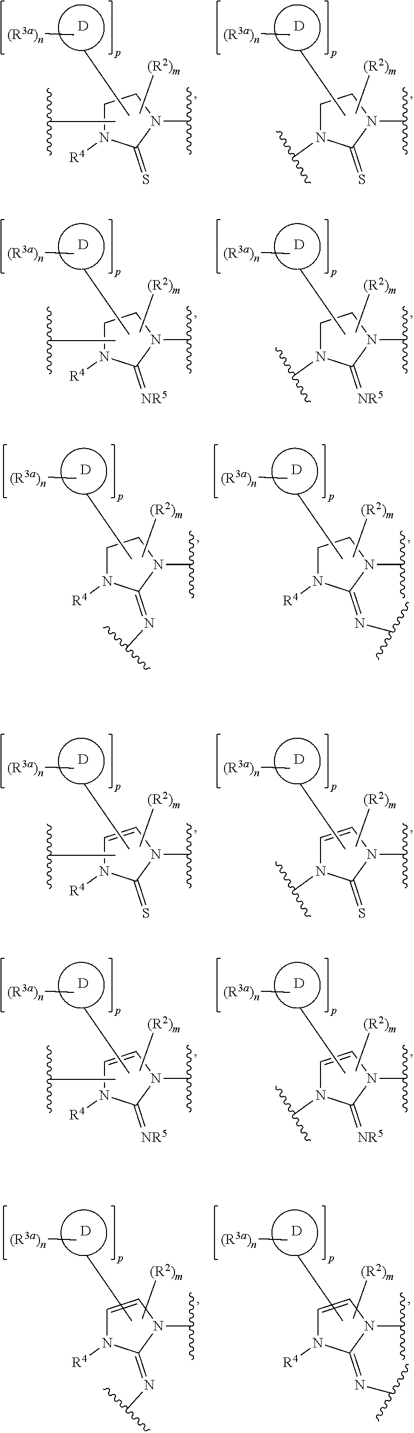

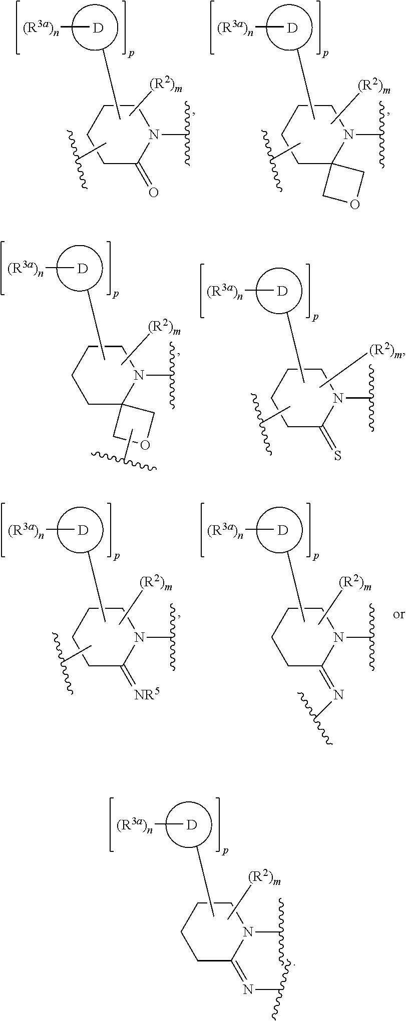

In some embodiments, Ring C is selected from

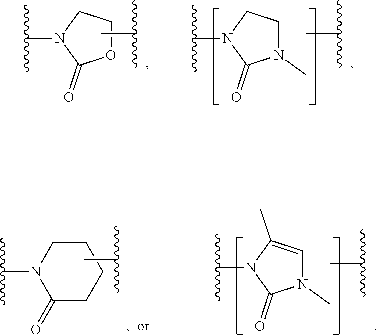

In some embodiments, Ring C is selected from

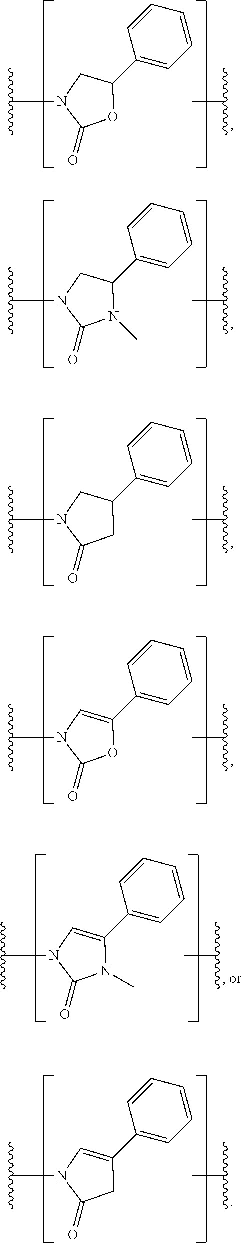

In some embodiments, Ring C is selected from those depicted in Table 1, below.

As defined above and described herein, Ring D is a ring selected from a 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring D is a 6 to 10-membered aryl. In some embodiments, Ring D is a 6 to 10-membered heteroaryl containing 1-4 heteroatoms independently selected from nitrogen, oxygen, and sulfur. In some embodiments, Ring D is a 5 to 7-membered saturated or partially unsaturated carbocyclyl. In some embodiments, Ring D is 5 to 7-membered saturated or partially saturated heterocyclyl with 1-3 heteroatoms independently selected from boron, nitrogen, oxygen, silicon, and sulfur. In some embodiments, Ring D is 5-membered heteroaryl with 1-4 heteroatoms independently selected from boron, nitrogen, oxygen, silicon, and sulfur.

In some embodiments, Ring D is quinoline. In some embodiments, Ring D is isoquinoline. In some embodiments, Ring D is imidazo[1,2-a]pyridine. In some embodiments, Ring D is indazole.

In some embodiments, Ring D is selected from those depicted in Table 1 below.

As defined above and described herein, each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups.

In some embodiments, one or more of Ring E, Ring F, and Ring G is a 6-membered aryl. In some embodiments, one or more of Ring E, Ring F, and Ring G is a 6-membered heteroaryl containing 1-4 heteroatoms independently selected from nitrogen, oxygen, and sulfur. In some embodiments, one or more of Ring E, Ring F, and Ring G is a 5 to 7-membered saturated or partially unsaturated carbocyclyl. In some embodiments, one or more of Ring E, Ring F, and Ring G is a 5 to 7-membered saturated or partially unsaturated heterocyclyl with 1-3 heteroatoms independently selected from boron, nitrogen, oxygen, silicon, and sulfur. In some embodiments, one or more of Ring E, Ring F, and Ring G is a 5-membered heteroaryl with 1-4 heteroatoms independently selected from nitrogen, oxygen and sulfur. In some embodiments, one or more of Ring E, Ring F, and Ring G is and optionally further substituted with 1-2 oxo groups.

In some embodiments, Ring E, Ring F, and Ring G are selected from those depicted in Table 1, below.

As defined above and described herein, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups.

In some embodiments, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring E and Ring H is selected from those depicted in Table 1, below.

As defined above and described herein, each of Ring I and Ring J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur

In some embodiments, each of Ring I and Ring J is independently a 6-membered aryl. In some embodiments, each of Ring I and Ring J is independently a 6-membered heteroaryl containing 1-4 heteroatoms independently selected from nitrogen, oxygen, and sulfur. In some embodiments, each of Ring I and Ring J is independently a 5 to 7-membered saturated or partially unsaturated carbocyclyl. In some embodiments, each of Ring I and Ring J is independently a 5 to 7-membered saturated or partially unsaturated heterocyclyl ring with 1-3 heteroatoms independently selected from boron, nitrogen, oxygen, silicon, and sulfur. In some embodiments, each of Ring I and Ring J is independently a 5-membered heteroaryl with 1-3 heteroatoms independently selected from nitrogen, oxygen and sulfur.

As defined above and described herein, Ring K is a fused ring selected from a 6-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring K is a fused ring selected from a 6-12 membered saturated or partially unsaturated carbocyclyl. In some embodiments, Ring K is a 6-12 membered saturated or partially unsaturated heterocyclyl ring with 1-3 heteroatoms independently selected from boron, nitrogen, oxygen, silicon, and sulfur. In some embodiments, Ring K is optionally further substituted with 1-2 oxo groups.

In some embodiments, Ring I, Ring J, and Ring K is selected from those depicted in Table 1, below.

As defined above and described herein, Ring M is selected from

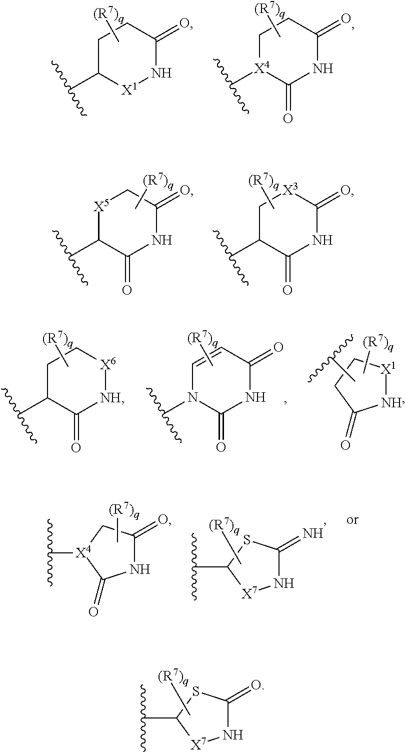

In some embodiments, Ring M is

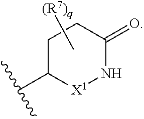

In some embodiments, Ring M is

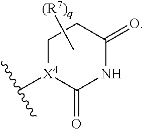

In some embodiments, Ring M is

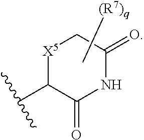

In some embodiments, Ring M is

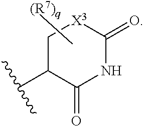

In some embodiments, Ring M is

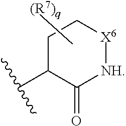

In some embodiments, Ring M is

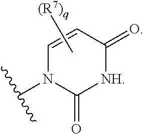

In some embodiments, Ring M is

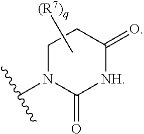

In some embodiments, Ring M is

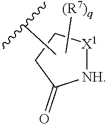

In some embodiments, Ring M is

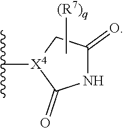

In some embodiments, Ring M is

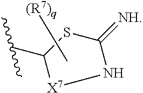

In some embodiments, Ring M is

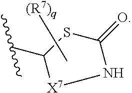

In some embodiments, Ring M is selected from those depicted in Table 1 below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;

In some embodiments, L¹ is a covalent bond. In some embodiments, L¹ is a C₁₋₃ aliphatic. In some embodiments, L¹ is —CH₂—. In some embodiments, L¹ is —C(D)(H)—. In some embodiments, L¹ is —C(D)₂—. In some embodiments, L¹ is —CH₂CH₂—. In some embodiments, L¹ is —NR—. In some embodiments, L¹ is —CH₂NR—. In some embodiments, L¹ is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—.

In some embodiments, Ring L¹ is selected from those depicted in Table 1, below.

As defined above and described herein, [symbol] is a single or double bond.

In some embodiments, [symbol] is a single bond. In some embodiments, [symbol] is a double bond.

In some embodiments, [symbol] is selected from those depicted in Table 1, below.

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1, below.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In some embodiments, n is selected from those depicted in Table 1, below.

As defined above and described herein, p is 0 or 1.

In some embodiments, p is 0. In some embodiments, p is 1.

In some embodiments, p is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiments, LBM is

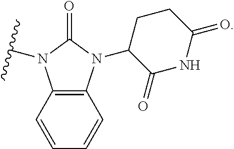 [SMILES: *n1c(=O)n(C2CCC(=O)NC2=O)c2ccccc21]

In some embodiments, LBM is

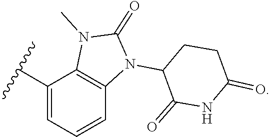 [SMILES: *c1cccc2c1n(C)c(=O)n2C1CCC(=O)NC1=O]

In some embodiments, LBM is

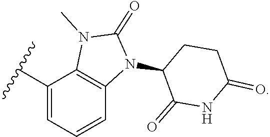 [SMILES: *c1cccc2c1n(C)c(=O)n2[C@H]1CCC(=O)NC1=O]

In some embodiments, LBM is

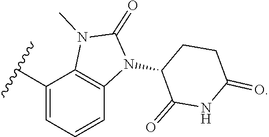 [SMILES: *c1cccc2c1n(C)c(=O)n2[C@@H]1CCC(=O)NC1=O]

In some embodiments, LBM is

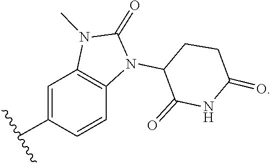 [SMILES: *c1ccc2c(c1)n(C)c(=O)n2C1CCC(=O)NC1=O]

In some embodiments, LBM is

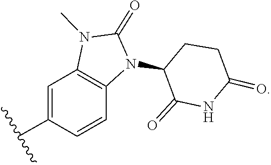 [SMILES: *c1ccc2c(c1)n(C)c(=O)n2[C@H]1CCC(=O)NC1=O]

In some embodiments, LBM is

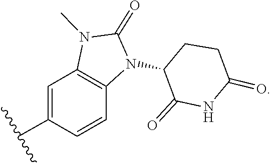 [SMILES: *c1ccc2c(c1)n(C)c(=O)n2[C@@H]1CCC(=O)NC1=O]

In some embodiments, LBM is

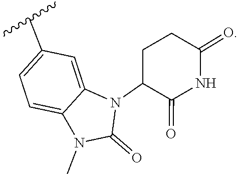 [SMILES: *c1ccc2c(c1)n(C1CCC(=O)NC1=O)c(=O)n2C]

In some embodiments, LBM is

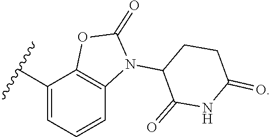 [SMILES: *c1cccc2c1oc(=O)n2C1CCC(=O)NC1=O]

In some embodiments, LBM is

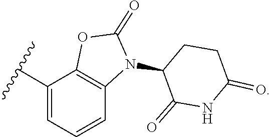 [SMILES: *c1cccc2c1oc(=O)n2[C@H]1CCC(=O)NC1=O]

In some embodiments, LBM is

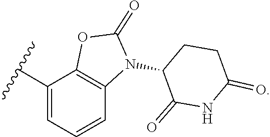 [SMILES: *c1cccc2c1oc(=O)n2[C@@H]1CCC(=O)NC1=O]

In some embodiments, LBM is

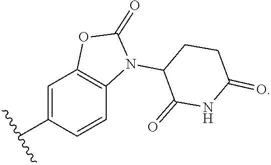 [SMILES: *c1ccc2c(c1)oc(=O)n2C1CCC(=O)NC1=O]

In some embodiments, LBM is

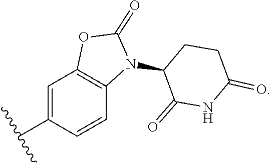 [SMILES: *c1ccc2c(c1)oc(=O)n2[C@H]1CCC(=O)NC1=O]

In some embodiments, LBM is

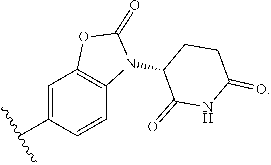 [SMILES: *c1ccc2c(c1)oc(=O)n2[C@@H]1CCC(=O)NC1=O]

In some embodiments, LBM is

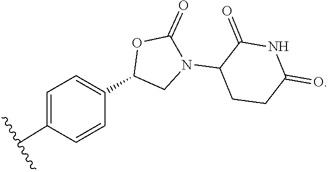 [SMILES: *c1ccc([C@H]2CN(C3CCC(=O)NC3=O)C(=O)O2)cc1]

In some embodiments, LBM is

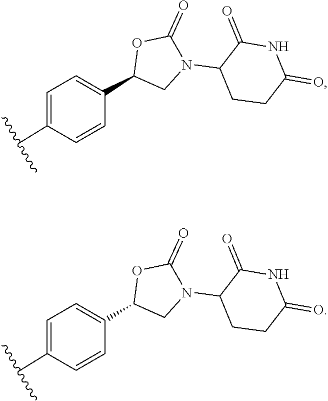 [SMILES: *c1ccc([C@@H]2CN(C3CCC(=O)NC3=O)C(=O)O2)cc1 ; *c1ccc([C@H]2CN(C3CCC(=O)NC3=O)C(=O)O2)cc1]

In some embodiments, LBM is

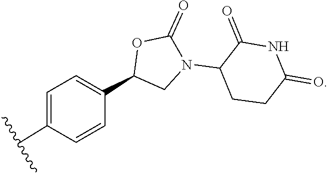 [SMILES: *c1ccc([C@@H]2CN(C3CCC(=O)NC3=O)C(=O)O2)cc1]

In some embodiments, LBM is

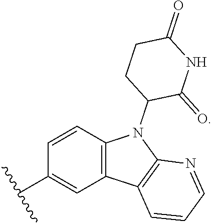 [SMILES: *c1ccc2c(c1)c1cccnc1n2C1CCC(=O)NC1=O]

In some embodiments, LBM is

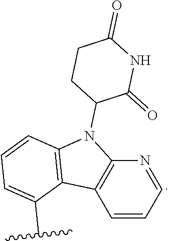 [SMILES: *c1cccc2c1c1cccnc1n2C1CCC(=O)NC1=O]

In some embodiments, LBM is

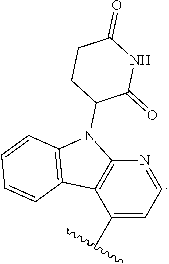 [SMILES: *c1ccnc2c1c1ccccc1n2C1CCC(=O)NC1=O]

In some embodiments, LBM is

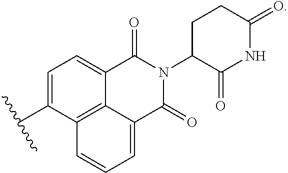 [SMILES: *c1ccc2c3c(cccc13)C(=O)N(C1CCC(=O)NC1=O)C2=O]

In some embodiments, LBM is

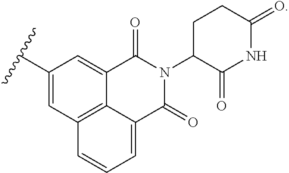 [SMILES: *c1cc2c3c(cccc3c1)C(=O)N(C1CCC(=O)NC1=O)C2=O]

In some embodiments, LBM is

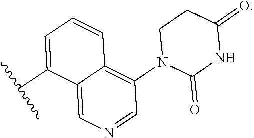 [SMILES: *c1cccc2c(N3CCC(=O)NC3=O)cncc12]

In some embodiments, LBM is

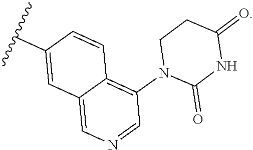 [SMILES: *c1ccc2c(N3CCC(=O)NC3=O)cncc2c1]

In some embodiments, LBM is

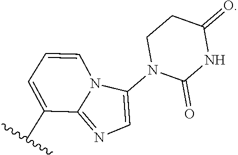 [SMILES: *c1cccn2c(N3CCC(=O)NC3=O)cnc12]

In some embodiments, LBM is

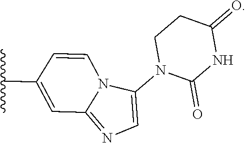 [SMILES: *c1ccn2c(N3CCC(=O)NC3=O)cnc2c1]

In some embodiments, LBM is

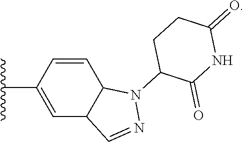 [SMILES: *C1=CC2C=NN(C3CCC(=O)NC3=O)C2C=C1]

In some embodiments, LBM is

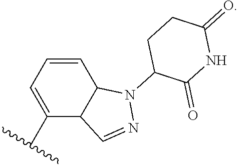 [SMILES: *C1=CC=CC2C1C=NN2C1CCC(=O)NC1=O]

In some embodiments, LBM is

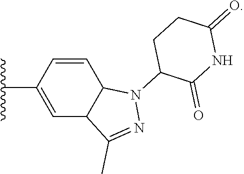 [SMILES: *C1=CC2C(C)=NN(C3CCC(=O)NC3=O)C2C=C1]

In some embodiments, LBM is

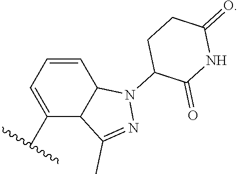 [SMILES: *C1=CC=CC2C1C(C)=NN2C1CCC(=O)NC1=O]

In certain embodiments, the present invention provides a compound of Formula I, wherein LBM is a MDM2 (i.e. human double minute 2 or HDM2) E3 ligase binding moiety thereby forming a compound of formula I-i-1, I-i-2, I-i-3, I-i-4, I-i-5, I-i-6, I-i-7, I-i-8, I-i-9, I-i-10, I-i-11, I-i-12, I-i-13, I-i-14, I-i-15, I-i-16, I-i-17, or I-i-18 respectively:

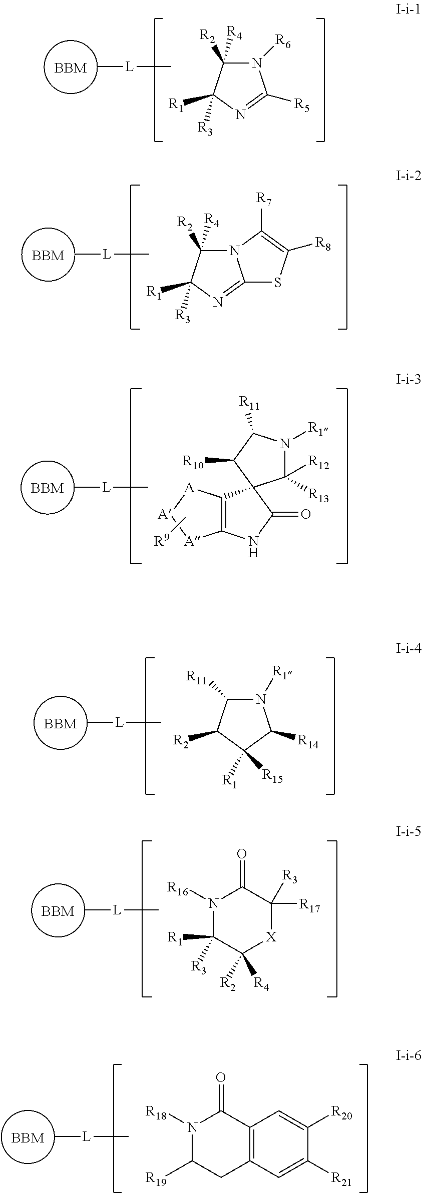

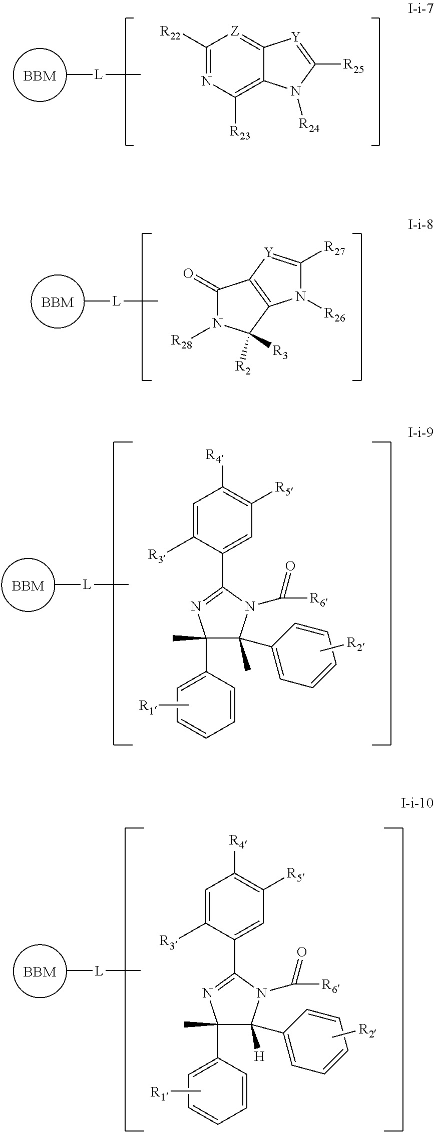

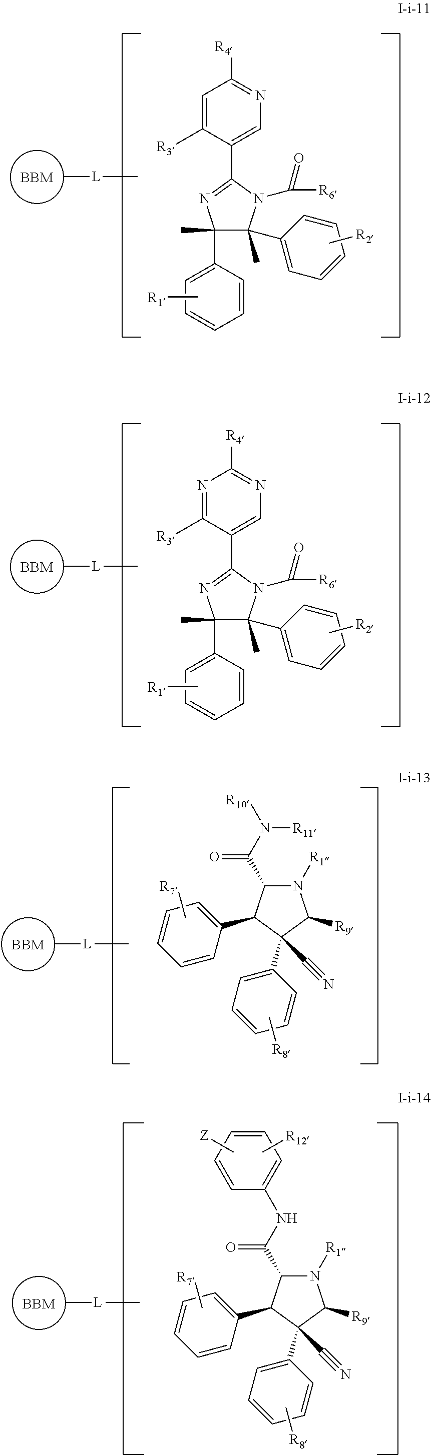

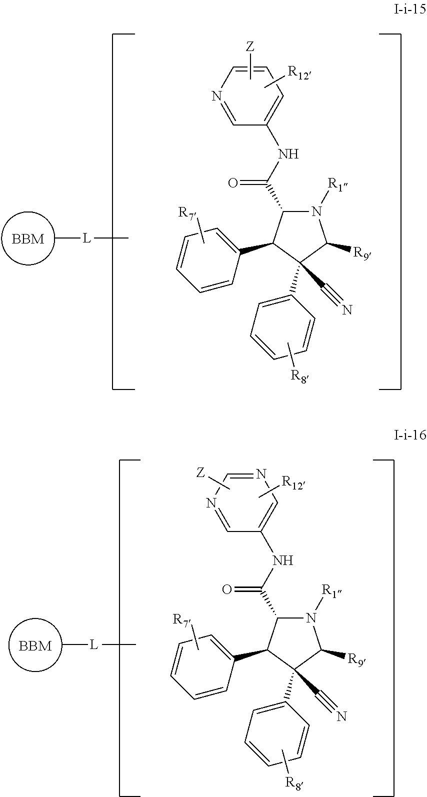

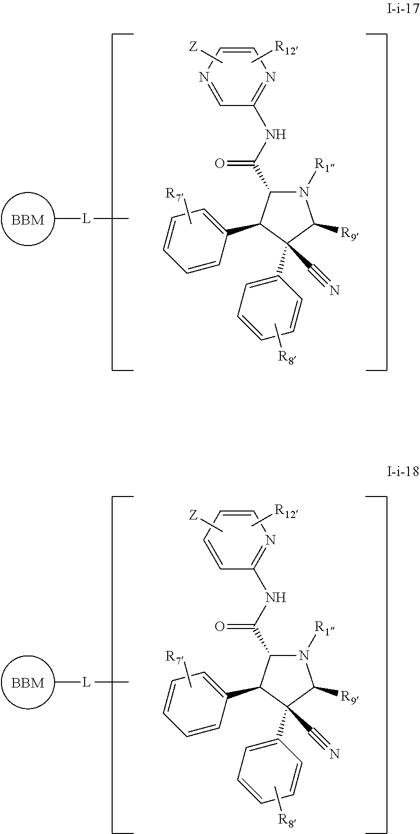

or a pharmaceutically acceptable salt thereof, wherein L and BBM are as defined above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═; Ring W is fused ring selected from benzo and a 5-6 membered heteroaryl with 1-4 heteroatoms independently selected from nitrogen, oxygen and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
   R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen, R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen, R^{A}, halogen, —C(O)R, —C(O)OR, —C(O)NR₂, —NR₂, —OR, —S(O)R, —S(O)₂R, —S(O)₂NR₂;
R²⁴, R²⁶ and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides a compound of Formula I, wherein LBM is a MDM2 (i.e. human double minute 2 or HDM2) E3 ligase binding moiety thereby forming a compound of formula I-i-19, I-i-20, or I-i-21 respectively:

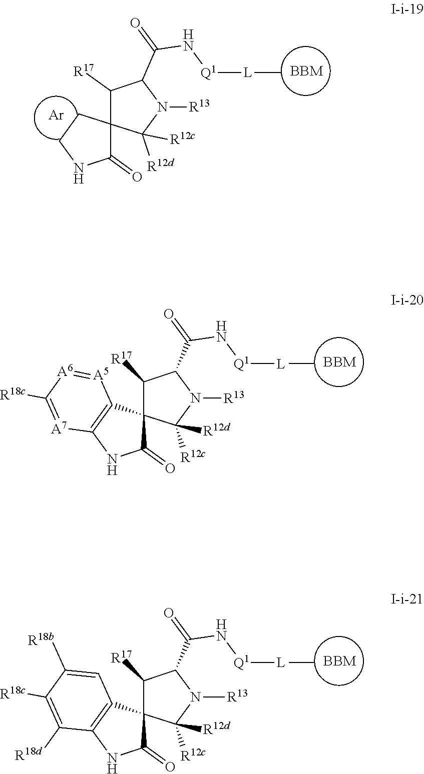

or a pharmaceutically acceptable salt thereof, wherein L and BBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹² and R¹³ are each independently selected from hydrogen and R^{A}, or:
   R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A5 is selected from —C(R^{18a})═ and —N═;
A6 is selected from —C(R^{18b})═ and —N═;
A7 is selected from —C(R^{18d})═ and —N═;
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen and sulfur; and
Q¹ is and optionally substituted bivalent group selected from alkylenyl, phenylenyl, heteroarylenyl, cycloalkylenyl, and heterocyclenyl.

In certain embodiments, the present invention provides a compound of formula I, wherein LBM is an IAP E3 ubiquitin ligase binding moiety thereby forming a compound of formula I-j-1, I-j-2, I-j-3, or I-j-4 respectively:

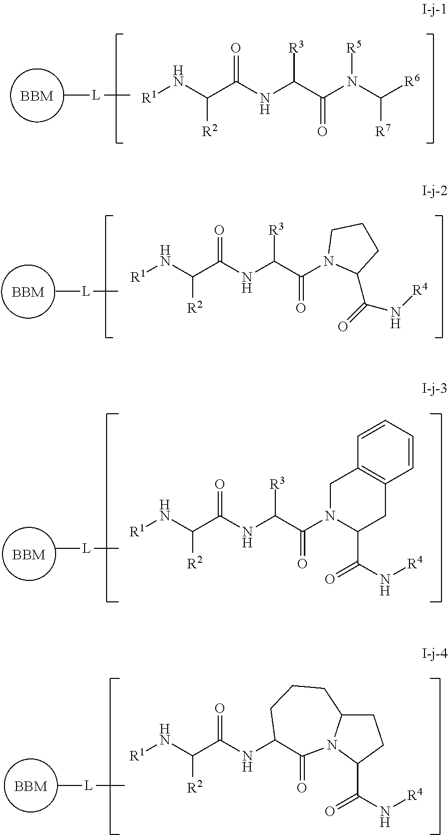

or a pharmaceutically acceptable salt thereof, wherein L and BBM are as defined above and described in embodiments herein, and wherein each of the variables R¹, R², R³, R⁴, R⁵, R⁶, and R⁷, is as defined and described in WO 2017/011590 and US 2017/0037004, the entirety of each of which is herein incorporated by reference.

In certain embodiments, the present invention provides a compound of formula I, wherein LBM is an IAP binding moiety thereby forming a compound of formula I-k-1:

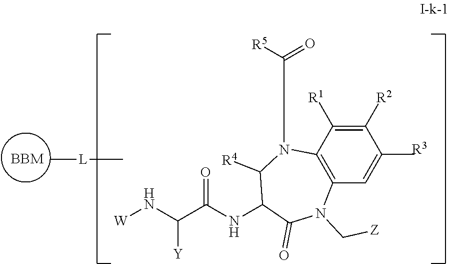

or a pharmaceutically acceptable salt thereof, wherein L and BBM are as defined above and described in embodiments herein, and wherein each of the variables W, Y, Z, R¹, R², R³, R⁴, and R⁵ is as described and defined in WO 2014/044622, US 2015/0225449. WO 2015/071393, and US 2016/0272596, the entirety of each of which is herein incorporated by reference.

In certain embodiments, the present invention provides a compound of formula I, wherein LBM is a DCAF16 binding moiety thereby forming a compound of formula I-k-2:

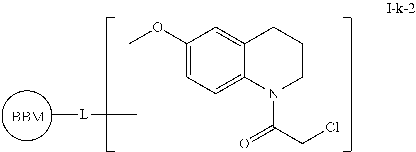

or a pharmaceutically acceptable salt thereof as described and defined in Zhang, X, et al., bioRxiv (doi: https://doi.org/10.1101/443804), the entirety of each of which is herein incorporated by reference, and wherein L and BBM are as defined above and described in embodiments herein.

In certain embodiments, the present invention provides a compound of formula I, wherein LBM is a RNF114 binding moiety thereby forming a compound of formula I-k-3:

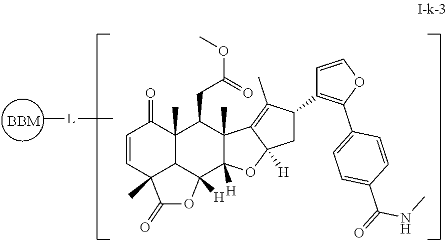

or a pharmaceutically acceptable salt thereof, as described and defined in Spradin, J. N. et al., bioRxiv (doi: https://doi.org/10.1101/436998), the entirety of each of which is herein incorporated by reference, and wherein L and BBM are as defined above and described in embodiments herein.

In certain embodiments, the present invention provides a compound of formula I, wherein LBM is a RNF4 binding moiety thereby forming a compound of formula I-k-4:

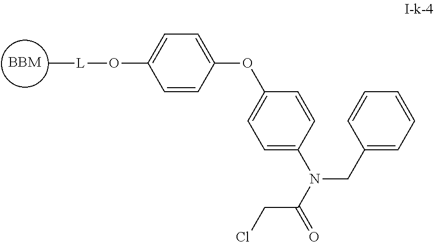

or a pharmaceutically acceptable salt thereof, as described and defined in Ward, C. C., et al., bioRxiv (doi: https://doi.org/10.1101/439125), the entirety of each of which is herein incorporated by reference, and wherein L and BBM are as defined above and described in embodiments herein.

In certain embodiments, the present invention provides a compound of formula I, wherein LBM is a E3 ubiquitin ligase (cereblon) binding moiety thereby forming a compound of formula I-I-1, I-I-2, I-1-3, or I-I-4:

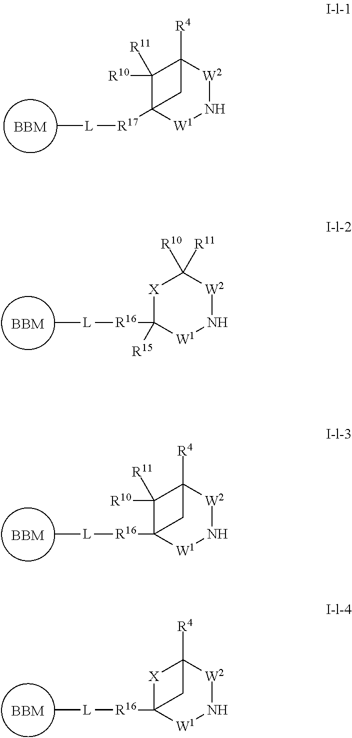

or a pharmaceutically acceptable salt thereof, wherein L and BBM are as defined above and described herein, and wherein each of the variables R⁴, R¹⁰, R¹¹, R¹⁵, R¹⁶, R¹⁷, W¹, W², and X is as defined in WO 2019/099868 which is herein incorporated by reference in its entirety, and wherein

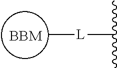

is attached to R¹⁷ or R¹⁶ at the site of attachment of R¹² as defined in WO 2018/237026, such that

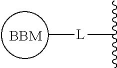

takes the place of the R¹² substituent.

In certain embodiments, the present invention provides a compound of formula I, wherein LBM is a cereblon E3 ubiquitin ligase binding moiety, a DCAF15 E3 ubiquitin ligase binding moiety, or a VHL E3 ubiquitin ligase binding moiety; thereby forming a compound of formula I-m-1, I-m-2, or I-m-3:

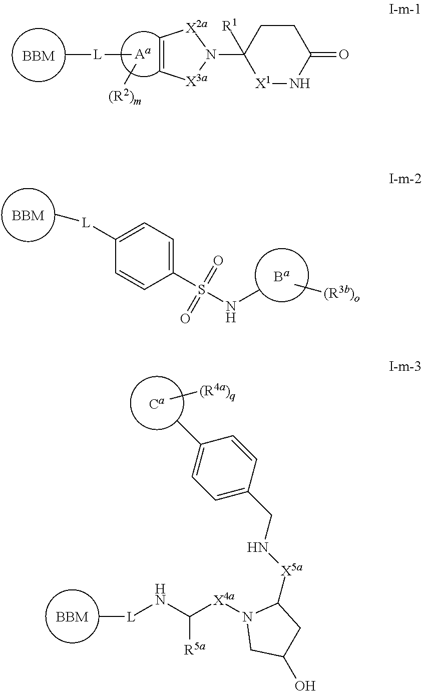

or a pharmaceutically acceptable salt thereof, wherein L and BBM is as defined above and described in embodiments herein, and wherein:
each of X¹, X^{2a}, and X^{3a} is independently a bivalent moiety selected from a covalent bond, —CH₂—, —C(O)—, —C(S)—, or

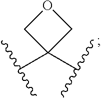 [SMILES: *C1(*)COC1]

each of X^{4a} and X^{5a} is independently a bivalent moiety selected from —CH₂—, —C(O)—, —C(S)—, or

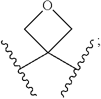 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur;
Ring B^{a} is selected from 6-membered aryl containing 0-2 nitrogen atoms or a 8-10 membered bicyclic heteroaryl having 1-5 heteroatoms independently selected from nitrogen, oxygen, and sulfur;
Ring C^{a} is a selected from 6-membered aryl containing 0-2 nitrogen atoms or a 5-membered heteroaryl with 1-3 heteroatoms independently selected from nitrogen, oxygen and sulfur;
m is 0, 1, 2, 3 or 4;
is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of formula I-m-1, wherein LBM is an E3 ubiquitin ligase (cereblon) binding moiety thereby forming a compound of formula I-m-4 or I-m-5:

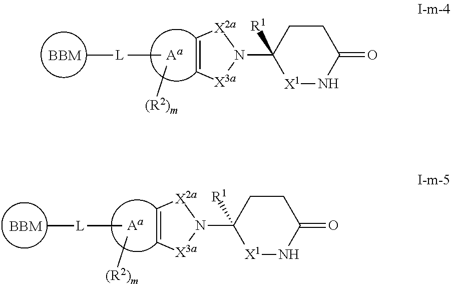

or a pharmaceutically acceptable salt thereof, wherein BBM, L, Ring Aa, X¹, X²a, X³a, R¹, R² and m are as described above.

As defined above and described herein, each of X¹, X^{2a}, and X^{3a} is independently a bivalent moiety selected from a covalent bond, —CH₂—, —C(O)—, —C(S)—, or

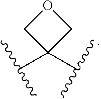 [SMILES: *C1(*)COC1]

In some embodiments, X¹ is a covalent bond, —CH₂—, —C(O)—, —C(S)—, or

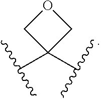 [SMILES: *C1(*)COC1]

In some embodiments, X¹ is selected from those depicted in Table 1, below.

In some embodiments, X^{2a} is a covalent bond, —CH₂—, —C(O)—, —C(S)—, or

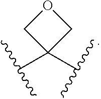 [SMILES: *C1(*)COC1]

In some embodiments, X^{2a} is selected from those depicted in Table 1, below.

In some embodiments, X^{3a} is a covalent bond, —CH₂—, —C(O)—, —C(S)—, or

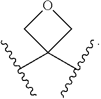 [SMILES: *C1(*)COC1]

In some embodiments, X^{3a} is selected from those depicted in Table 1, below.

As defined above and described herein, each of X^{4a} and X^{5a} is independently a bivalent moiety selected from —CH₂—, —C(O)—, —C(S)—, or

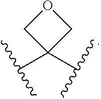 [SMILES: *C1(*)COC1]

In some embodiments, X^{4a} is —CH₂—, —C(O)—, —C(S)—, or

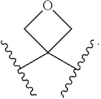 [SMILES: *C1(*)COC1]

In some embodiments, X^{4a} is selected from those depicted in Table 1, below.

In some embodiments, X^{5a} is —CH₂—, —C(O)—, —C(S)—, or

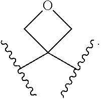 [SMILES: *C1(*)COC1]

In some embodiments, X^{5a} is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{5a} is hydrogen or C₁₋₆ aliphatic.

In some embodiments, R^{5a} is t-butyl.

In some embodiments, R^{5a} is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring Aa is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen and sulfur. In some embodiments Ring A^{a} is a fused 5-membered heteroaryl with 1-3 heteroatoms independently selected from nitrogen, oxygen and sulfur.

In some embodiments, Ring A^{a} is a fused phenyl.

In some embodiments, Ring A^{a} is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, and sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, and sulfur.

In some embodiments, Ring B^{a} is

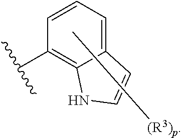

In some embodiments, Ring B^{a} is selected from those depicted in Table 1, below.

As defined above and described herein, Ring Ca is selected from 6-membered aryl containing 0-2 nitrogen atoms or a 5-membered heteroaryl with 1-3 heteroatoms independently selected from nitrogen, oxygen and sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen and sulfur.

In some embodiments, Ring C^{a} is

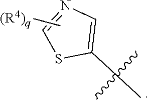

In some embodiments, Ring C^{a} is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of formula I, wherein LBM is a VHL E3 ubiquitin ligase binding moiety, thereby forming a compound of formula I-n:

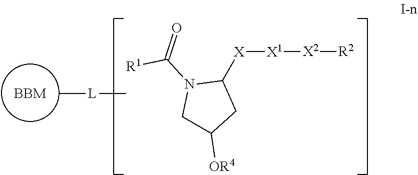

or a pharmaceutically acceptable salt thereof, wherein L and BBM is as defined above and described in embodiments herein, and wherein:
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
R^{A} is an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R² is hydrogen, halogen, —CN,

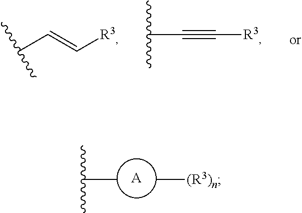

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R) OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R; or
   two R³ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R², —P(O)(OR)₂, —(CR₂)₁₋₃P(O)R², —(CR₂)₁₋₃P(O)(OR)₂, —(CR₂)₁₋₃OP(O)R², —(CR₂)₁₋₃OP(O)(OR)₂, —C(O)(CR₂)₁₋₃P(O)R², —C(O)(CR₂)₁₋₃P(O)(OR)₂, —C(O)(CR₂)₁₋₃OP(O)R², —C(O)(CR₂)₁₋₃OP(O)(OR)₂, or R^{A};
n is 0, 1, 2, 4, or 5.

As defined above and described herein, in some embodiments, X is —C(O)—, —C(O)NR—, —SO₂—, —SO₂NR—, or an optionally substituted 5-membered heterocyclic ring.

In some embodiments, X is —C(O)—. In some embodiments, X is —C(O)NR—. In some embodiments, X is —SO₂—. In some embodiments, X is —SO₂NR—. In some embodiments, X is an optionally substituted 5-membered heterocyclic ring.

In some embodiments, X is —C(O)NH—. In some embodiments, X is

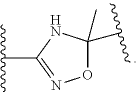 [SMILES: *C1=NOC(*)(C)N1]

In some embodiments, X is selected from those depicted in Table 1, below.

As defined above and described herein, in some embodiments, X¹ is a bivalent group selected from a covalent bond, —O—, —C(O)—, —C(S)—, —C(R)₂—, —NR—, —S(O)—, or —SO₂—.

In some embodiments, X¹ is a covalent bond. In some embodiments, X¹ is —O—. In some embodiments, X¹ is —C(O)—. In some embodiments, X¹ is —C(S)—. In some embodiments, X¹ is —C(R)₂—. In some embodiments, X¹ is —NR—. In some embodiments, X¹ is —S(O)—. In some embodiments, X¹ is —SO₂—.

In some embodiments, X¹ is

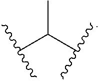 [SMILES: *C(*)C]

In some embodiments, X¹ is

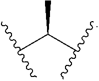

In some embodiments, X¹ is

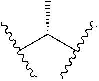

In some embodiments, X¹ is

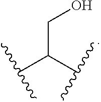 [SMILES: *C(*)CO]

In some embodiments, X¹ is

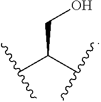

In some embodiments, X¹ is

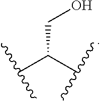

In some embodiments, X¹ is

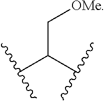 [SMILES: *C(*)COC]

In some embodiments, X¹ is

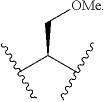

In some embodiments, X¹ is

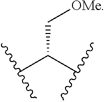

In some embodiments, X¹ is

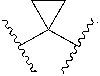 [SMILES: *C1(*)CC1]

In some embodiments, X¹ is selected from those depicted in Table 1, below.

As defined above and described herein, in some embodiments, X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, X² is an optionally substituted C₁₋₆ saturated or unsaturated alkylene. In some embodiments, X² is an optionally substituted phenylenyl. In some embodiments, X² is an optionally substituted 5-9 membered heteroarylenyl containing 1-4 heteroatoms independently selected from nitrogen, oxygen, and sulfur. In some embodiments, X² is an optionally substituted 4-11 membered saturated or partially unsaturated monocyclic, bicyclic, bridged bicyclic, or spirocyclic carbocyclylenyl. In some embodiments, X² is an optionally substituted 4-11 membered saturated or partially unsaturated monocyclic, bicyclic, bridged bicyclic, or spirocyclic heterocyclylenyl with 1-3 heteroatoms independently selected from nitrogen, oxygen, and sulfur.

In some embodiments, X² is

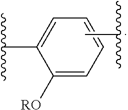

In some embodiments, X² is

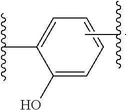

In some embodiments, X² is

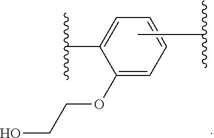

In some embodiments, X² is

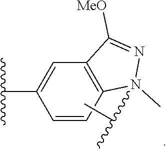

In some embodiments, X² is

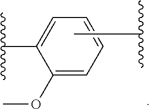

In some embodiments, X² is

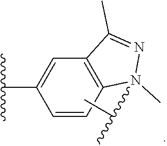

In some embodiments, X² is selected from those depicted in Table 1, below.

As defined above and described herein, in some embodiments, R¹ is R^{z}, —C(R)₂R², —OR, —SR, —N(R)₂, —C(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R.

In some embodiments, R¹ is R². In some embodiments, R¹ is —C(R)₂R^{z}. In some embodiments, R¹ is —OR. In some embodiments, R¹ is —SR. In some embodiments, R¹ is —N(R)₂. In some embodiments, R¹ is —C(R)₂OR. In some embodiments, R¹ is —C(R)₂N(R)₂. In some embodiments, R¹ is —C(R)₂NRC(O)R. In some embodiments, R¹ is —C(R)₂NRC(O)N(R)₂. In some embodiments, R¹ is —NRC(O)OR. In some embodiments, R¹ is —NRC(O)R. In some embodiments, R¹ is —NRC(O)N(R)₂. In some embodiments, R¹ is —NRSO₂R.

In some embodiments, R¹ is

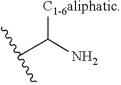

In some embodiments, R¹ is

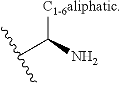

In some embodiments, R¹ is

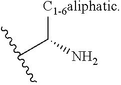

In some embodiments, R¹ is

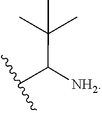 [SMILES: *C(N)C(C)(C)C]

In some embodiments, R¹ is

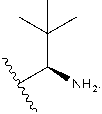 [SMILES: *[C@H](N)C(C)(C)C]

In some embodiments, R¹ is

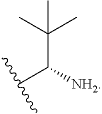 [SMILES: *[C@@H](N)C(C)(C)C]

In some embodiments, R¹ is

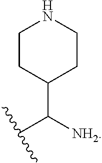 [SMILES: *C(N)C1CCNCC1]

In some embodiments, R¹ is

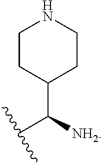 [SMILES: *[C@H](N)C1CCNCC1]

In some embodiments, R¹ is

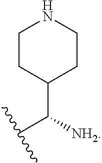 [SMILES: *[C@@H](N)C1CCNCC1]

In some embodiments, R¹ is

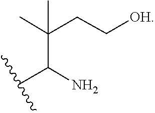 [SMILES: *C(N)C(C)(C)CCO]

In some embodiments, R¹ is

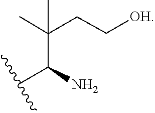 [SMILES: *[C@H](N)C(C)(C)CCO]

In some embodiments, R¹ is

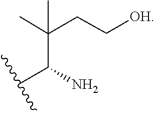 [SMILES: *[C@@H](N)C(C)(C)CCO]

In some embodiments, R¹ is

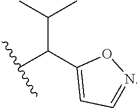 [SMILES: *C(c1ccno1)C(C)C]

In some embodiments, R¹ is

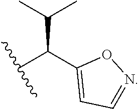 [SMILES: *[C@H](c1ccno1)C(C)C]

In some embodiments, R¹ is

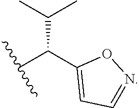 [SMILES: *[C@@H](c1ccno1)C(C)C]

In some embodiments, R¹ is

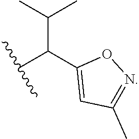 [SMILES: *C(c1cc(C)no1)C(C)C]

In some embodiments, R¹ is

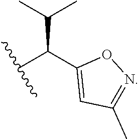 [SMILES: *[C@H](c1cc(C)no1)C(C)C]

In some embodiments, R¹ is

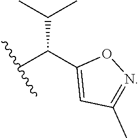 [SMILES: *[C@@H](c1cc(C)no1)C(C)C]

In some embodiments, R¹ is

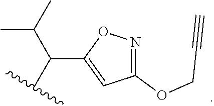 [SMILES: *C(c1cc(OCC#C)no1)C(C)C]

In some embodiments, R¹ is

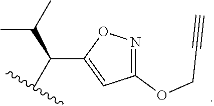 [SMILES: *[C@H](c1cc(OCC#C)no1)C(C)C]

In some embodiments, R¹ is

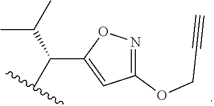 [SMILES: *[C@@H](c1cc(OCC#C)no1)C(C)C]

In some embodiments, R¹ is

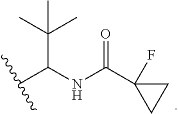 [SMILES: *C(NC(=O)C1(F)CC1)C(C)(C)C]

In some embodiments, R¹ is

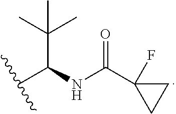 [SMILES: *[C@H](NC(=O)C1(F)CC1)C(C)(C)C]

In some embodiments, R¹ is

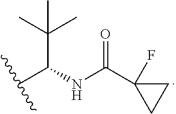 [SMILES: *[C@@H](NC(=O)C1(F)CC1)C(C)(C)C]

In some embodiments, R¹ is

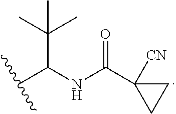 [SMILES: *C(NC(=O)C1(C#N)CC1)C(C)(C)C]

In some embodiments, R¹ is

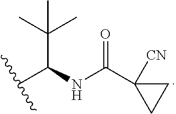 [SMILES: *[C@H](NC(=O)C1(C#N)CC1)C(C)(C)C]

In some embodiments, R¹ is

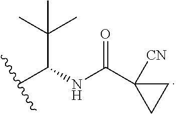 [SMILES: *[C@@H](NC(=O)C1(C#N)CC1)C(C)(C)C]

In some embodiments, R¹ is

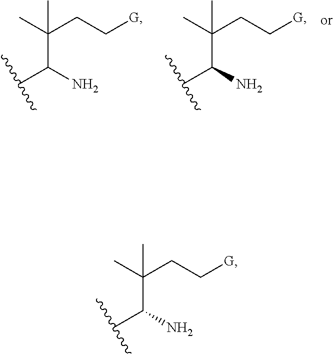

wherein G is —OH, —O(CH₂)₁₋₅CO₂R (e.g., —OCH₂CO₂H, etc.)), —OP(O)(OR)₂ (e.g., —OP(O)(OH)₂, etc.)), —O(CH₂)₁—SP(O)(OR)₂ (e.g., —O(CH₂)₂P(O)(OH)₂, etc.)),

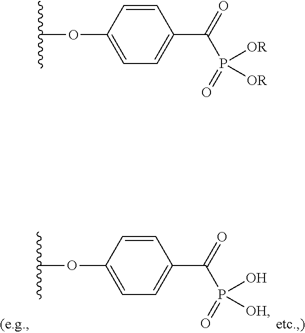

or —NR₂ (e.g., —NMe₂,

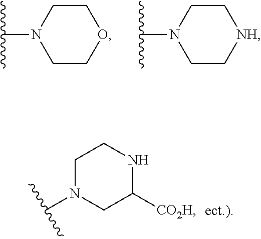

In some embodiments, R¹ is

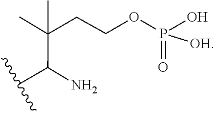 [SMILES: *C(N)C(C)(C)CCOP(=O)(O)O]

In some embodiments, R¹ is

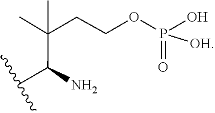 [SMILES: *[C@H](N)C(C)(C)CCOP(=O)(O)O]

In some embodiments, R¹ is

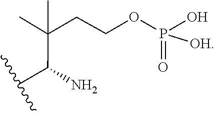 [SMILES: *[C@@H](N)C(C)(C)CCOP(=O)(O)O]

In some embodiments, R¹ is selected from those depicted in Table 1, below.

As defined above and described herein, in some embodiments, R² is hydrogen, halogen, —CN,

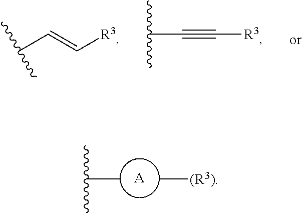

In some embodiments, R² is hydrogen. In some embodiments, R² is halogen. In some embodiments, R² is —CN. In some embodiments, R² is

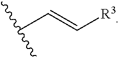

In some embodiments, R² is

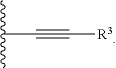

In some embodiments, R² is

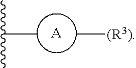

In some embodiments, R² is fluoro. In some embodiments, R² is chloro. In some embodiments, R² is

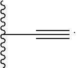 [SMILES: *C#C]

In some embodiments, R² is selected from those depicted in Table 1, below.

As defined above and described herein, in some embodiments, Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring A is phenyl. In some embodiments, Ring A is a 5-6 membered heteroaryl containing 1-4 heteroatoms independently selected from nitrogen, oxygen, and sulfur. In some embodiments, Ring A is 4 to 9-membered saturated or partially unsaturated monocyclic, bicyclic, bridged bicyclic, or spirocyclic carbocyclyl. In some embodiments, Ring A is a 4 to 9-membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring A is

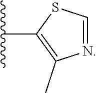 [SMILES: *c1scnc1C]

In some embodiments, Ring A is

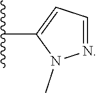 [SMILES: *c1ccnn1C]

In some embodiments, Ring A is selected from those depicted in Table 1, below.

As defined above and described herein, in some embodiments, each of R³ is independently hydrogen, R^{z}, halogen, —CN, —NO₂, —OR, —SR, —N(R)₂, —Si(R)₃, —SO₂R, —SO₂NR₂, —S(O)R, —C(O)R, —C(O)OR, —C(O)N(R)₂, —C(O)N(R) OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R, or two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is hydrogen. In some embodiments, R³ is R². In some embodiments, R³ is halogen. In some embodiments, R³ is —CN. In some embodiments, R³ is —NO₂. In some embodiments, R³ is —OR. In some embodiments, R³ is —SR. In some embodiments, R³ is —N(R)₂. In some embodiments, R³ is —Si(R)₃. In some embodiments, R³ is —SO₂R. In some embodiments, R³ is —SO₂NR₂. In some embodiments, R³ is —S(O)R. In some embodiments, R³ is —C(O)R. In some embodiments, R³ is —C(O)OR. In some embodiments, R³ is —C(O)N(R)₂. In some embodiments, R³ is —C(O)N(R) OR. In some embodiments, R³ is —C(R)₂NRC(O)R. In some embodiments, R³ is —C(R)₂NRC(O)N(R)₂. In some embodiments, R³ is —OC(O)R. In some embodiments, R³ is —OC(O)N(R)₂. In some embodiments, R³ is —OP(O)(R)₂. In some embodiments, R³ is —OP(O)(OR)₂. In some embodiments, R³ is —OP(O)(OR)N(R)₂. In some embodiments, R³ is —OP(O)(N(R)₂)₂—. In some embodiments, R³ is —N(R)C(O)OR. In some embodiments, R³ is —N(R)C(O)R. In some embodiments, R³ is —NRC(O)N(R)₂. In some embodiments, R³ is —N(R)SO₂R. In some embodiments, R³ is —NP(O)(R)₂. In some embodiments, R³ is —N(R)P(O)(OR)₂. In some embodiments, R³ is —N(R)P(O)(OR)N(R)₂. In some embodiments, R³ is —N(R)P(O)(N(R)₂)₂. In some embodiments, R³ is —N(R)SO₂R. In some embodiments, two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is methyl.

In some embodiments, R³ is selected from those depicted in Table 1, below.

As defined above and described herein, R⁴ is hydrogen, —C(O)R, —C(O)OR, —C(O)NR₂, —P(O)R₂, —P(O)(OR)₂, —(CR₂)₁—; P(O)R₂, —(CR₂)₁—; P(O)(OR)₂, —(CR₂)₁—; OP(O)R₂, —(CR₂)₁—; OP(O)(OR)₂, —C(O)(CR₂)₁—; P(O)R₂, —C(O)(CR₂)₁—; P(O)(OR)₂, —C(O)(CR₂)₁—; OP(O)R₂, —C(O)(CR₂)₁—; OP(O)(OR)₂, or R^{A}.

In some embodiments, R⁴ is hydrogen. In some embodiments, R⁴ is —C(O)R. In some embodiments, R⁴ is —C(O)OR. In some embodiments, R⁴ is —C(O)NR₂. In some embodiments, R⁴ is —P(O)R₂. In some embodiments, R⁴ is —P(O)(OR)₂. In some embodiments, R⁴ is —(CR₂)₁—; P(O)R₂. In some embodiments, R⁴ is —(CR₂)₁—; P(O)(OR)₂. In some embodiments, R⁴ is —(CR₂)₁—; OP(O)R₂. In some embodiments, R⁴ is —(CR₂)₁—; OP(O)(OR)₂. In some embodiments, R⁴ is —C(O)(CR₂)₁—; P(O)R₂. In some embodiments, R⁴ is —C(O)(CR₂)₁—; P(O)(OR)₂. In some embodiments, R⁴ is —C(O)(CR₂)₁—; OP(O)R₂. In some embodiments, R⁴ is —C(O)(CR₂)₁—; OP(O)(OR)₂. In some embodiments, R⁴ is R^{A}.

In some embodiments, R⁴ is —C(O)(CH₂)₂P(O)(OH)₂.

In some embodiments, R⁴ is selected from those depicted in Table 1, below.

As defined above and described herein, in some embodiments, n is 0, 1, 2, 4, or 5.

In some embodiments, n is 0. In some embodiments, n is 1. In some embodiments, n is 2. In some embodiments, n is 3. In some embodiments, n is 4. In some embodiments, n is 5.

In some embodiments, n is selected from those depicted in Table 1, below.

In certain embodiments, the present invention provides a compound of formula I-aa-1:

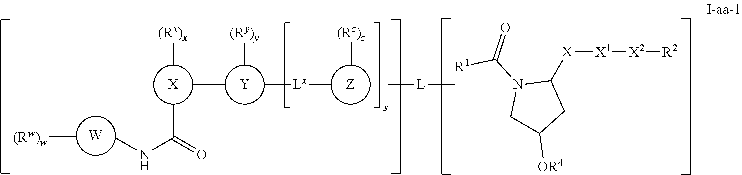

or a pharmaceutically acceptable salt, wherein:
Ring W and Ring Z are, independently, a ring selected from phenyl, naphthyl, a 5-10 membered heteroaryl containing 1-4 heteroatoms independently selected from nitrogen, oxygen, selenium, and sulfur, and a 4-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring X is a bicyclic ring selected from a 9-11 membered partially unsaturated heterocyclylenyl with 1-4 heteroatoms independently selected from nitrogen, oxygen, and sulfur, and a 9-10 membered heteroarylenyl containing 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R—S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of L¹ are independently replaced by optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s is 0 or 1; and
w, x, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

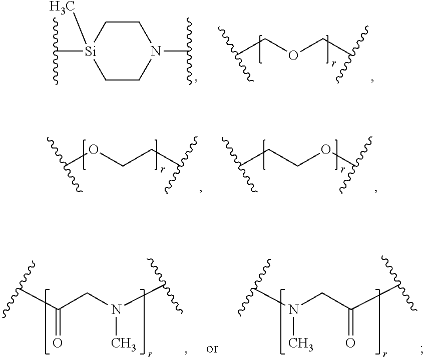

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R) ¿R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R₂ is hydrogen, halogen, —CN,

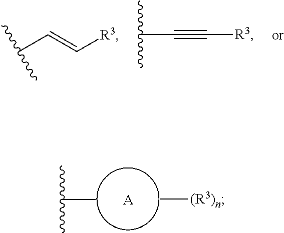

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R₃ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R) OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R: or
   two R₃ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃P(O)R₂, —(CR₂)₁₋₃P(O)(OR)₂, —(CR₂)₁₋₃OP(O)R₂, —(CR₂)₁₋₃OP(O)(OR)₂, —C(O)(CR₂)₁₋₃P(O)R₂, —C(O)(CR₂)₁₋₃P(O)(OR)₂, —C(O)(CR₂)₁₋₃OP(O)R₂, —C(O)(CR₂)₁₋₃OP(O)(OR)₂, or R^{A};
n is 0, 1, 2, 4, or 5.

In certain embodiments, the present invention provides a compound of formula I-aa-1, wherein

R¹ is

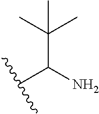 [SMILES: *C(N)C(C)(C)C]

(where one of the hydrogen atoms of the NH₂ group is replaced with -L-) as shown, to provide a compound of formula I-aa-2:

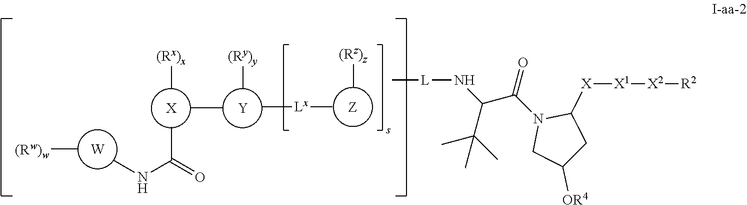

or a pharmaceutically acceptable salt thereof, wherein each of L, L^{x}, X, X¹, X², R², R⁴, R^{w}, R^{x}, R^{y}, R^{z}, Ring W, Ring X, Ring Y, Ring Z, s, v, w, x, y, and z is as defined above and described in embodiments herein, both singly and in combination.

In certain embodiments, the present invention provides a compound of formula I-aa-1, wherein

### R¹ is

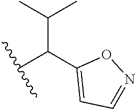 [SMILES: *C(c1ccno1)C(C)C]

(where one of the hydrogen atoms of the isoxazolyl group is replaced with -L-) as shown, to provide a compound of formula I-aa-3:

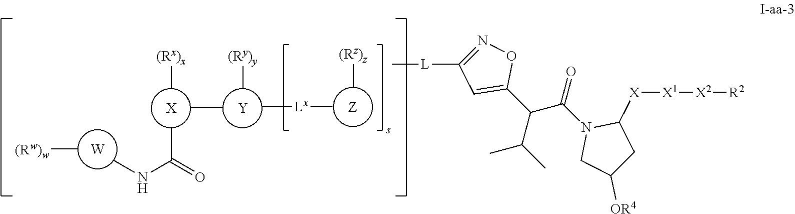

or a pharmaceutically acceptable salt thereof, wherein each of L, L^{x}, X, X¹, X², R², R⁴, R^{w}, R^{x}, R^{y}, R^{z}, Ring W, Ring X, Ring Y, Ring Z, s, v, w, x, y, and z is as defined above and described in embodiments herein, both singly and in combination.

In certain embodiments, the present invention provides a compound of formula I-aa-1, wherein R¹ is

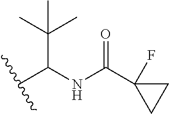 [SMILES: *C(NC(=O)C1(F)CC1)C(C)(C)C]

and X² is phenylenyl as shown, to provide a compound of formula I-aa-4:

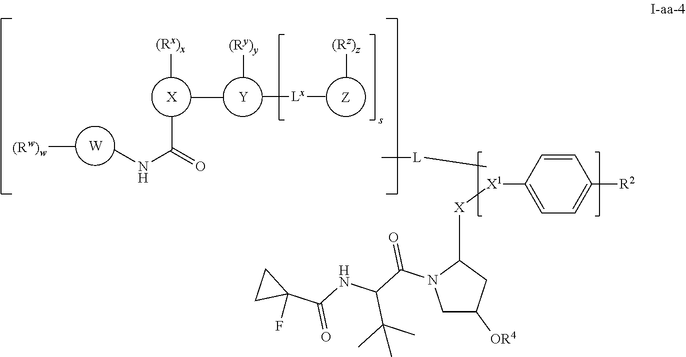

or a pharmaceutically acceptable salt thereof, wherein each of L, L^{x}, X, X¹, R², R₄, R^{w}, R^{x}, R^{y}, R^{z}, Ring W, Ring X, Ring Y, Ring Z, s, v, w, x, y, and z is as defined above and described in embodiments herein, both singly and in combination.

In certain embodiments, the present invention provides a compound of formula I-aa-1, wherein R¹ is

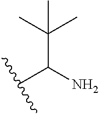 [SMILES: *C(N)C(C)(C)C]

(where one of the hydrogen atoms of the NH₂ group is replaced with -L-), Ring X is 1,2,3,4-tetrahydroisoquinolinylenyl, and Ring W is benzothiazolyl as shown, to provide a compound of formula I-aa-5:

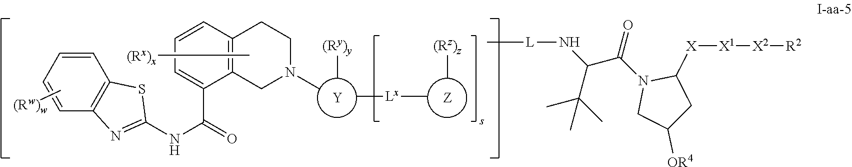

or a pharmaceutically acceptable salt thereof, wherein each of L, L^{x}, X, X¹, X², R², R⁴, R^{w}, R^{x}, R^{y}, R^{z}, Ring Y, Ring Z, s, v, w, x, y, and z is as defined above and described in embodiments herein, both singly and in combination.

In certain embodiments, the present invention provides a compound of formula I-aa-1, wherein R¹ is

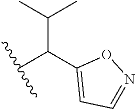 [SMILES: *C(c1ccno1)C(C)C]

(where one of the hydrogen atoms of the NH₂ group is replaced with -L-), Ring X is 1,2,3,4-tetrahydroisoquinolinylenyl, and Ring W is benzothiazolyl as shown, to provide a compound of formula I-aa-6:

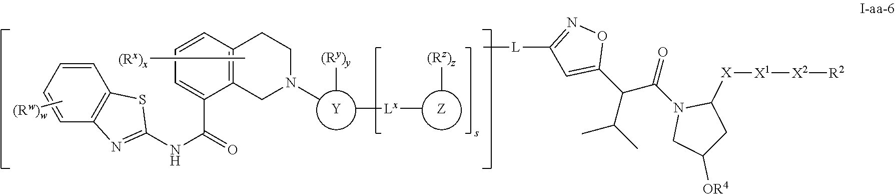

or a pharmaceutically acceptable salt thereof, wherein each of L, L^{x}, X, X¹, X², R², R⁴, R^{w}, R^{x}, R^{y}, R^{z}, Ring Y, Ring Z, s, v, w, x, y, and z is as defined above and described in embodiments herein, both singly and in combination.

In certain embodiments, the present invention provides a compound of formula I-aa-1, wherein R¹ is

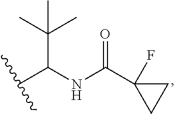 [SMILES: *C(NC(=O)C1(F)CC1)C(C)(C)C]

X² is phenylenyl, Ring X is 1,2,3,4-tetrahydroisoquinolinylenyl, and Ring W is benzothiazolyl as shown, to provide a compound of formula I-aa-7:

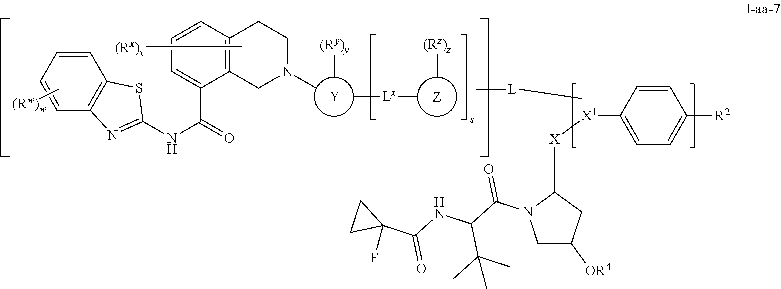

or a pharmaceutically acceptable salt thereof, wherein each of L, L^{x}, X, X¹, R², R₄, R^{w}, R^{x}, R^{y}, R^{z}, Ring Y, Ring Z, s, v, w, x, y, and z is as defined above and described in embodiments herein, both singly and in combination.

In certain embodiments, the present invention provides a compound of formula I-aa-1, wherein R¹ is

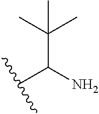 [SMILES: *C(N)C(C)(C)C]

(where one of the hydrogen atoms of the NH₂ group is replaced with -L-), Ring X is 1,2,3,4-tetrahydroisoquinolinylenyl, and Ring W is benzothiazolyl as shown, to provide a compound of formula I-aa-8:

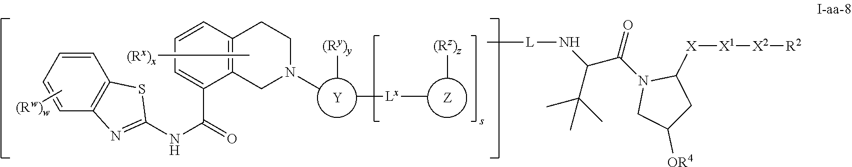

or a pharmaceutically acceptable salt thereof, wherein:
L does not connect (e.g., covalently bond) to BBM through

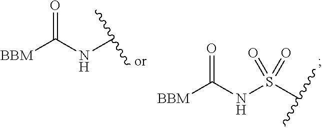

and each of L, L^{x}, X, X¹, X², R², R⁴, R^{w}, R^{x}, R^{y}, R², Ring Y, Ring Z, s, v, w, x, y, and z is as defined above and described in embodiments herein, both singly and in combination.

In certain embodiments, the present invention provides a compound of formula I-aa-1, wherein R¹ is

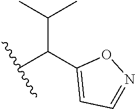 [SMILES: *C(c1ccno1)C(C)C]

(where one of the hydrogen atoms of the NH₂ group is replaced with -L-), Ring X is 1,2,3,4-tetrahydroisoquinolinylenyl, and Ring W is benzothiazolyl as shown, to provide a compound of formula I-aa-9:

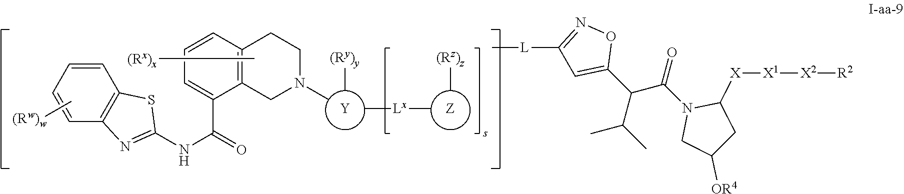

or a pharmaceutically acceptable salt thereof, wherein:
L does not connect (e.g., covalently bond) to BBM through

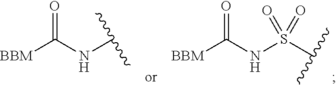

and each of L, L^{x}, X, X¹, X², R², R⁴, R^{w}, R^{x}, R^{y}, R^{z}, Ring Y, Ring Z, s, v, w, x, y, and z is as defined above and described in embodiments herein, both singly and in combination.

In certain embodiments, the present invention provides a compound of formula I-aa-1, wherein R¹ is

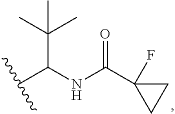 [SMILES: *C(NC(=O)C1(F)CC1)C(C)(C)C]

X² is phenylenyl, Ring X is 1,2,3,4-tetrahydroisoquinolinylenyl, and Ring W is benzothiazolyl as shown, to provide a compound of formula I-aa-10:

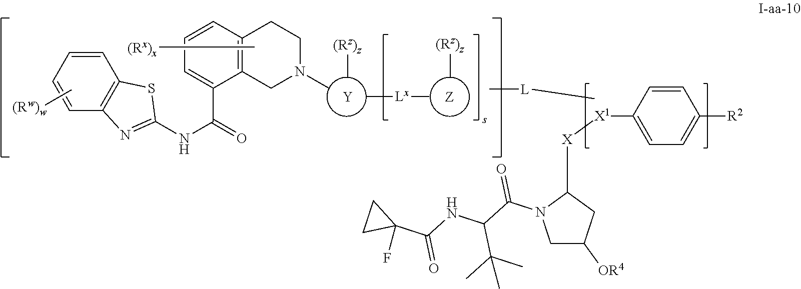

or a pharmaceutically acceptable salt thereof, wherein:
L does not connect (e.g., covalently bond) to BBM through

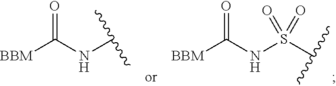

and each of L, L^{x}, X, X¹, R², R₄, R^{w}, R^{x}, R^{y}, R², Ring Y, Ring Z, s, v, w, x, y, and z is as defined above and described in embodiments herein, both singly and in combination.

In some embodiments, L does not connect (e.g., covalently bond) to BBM through

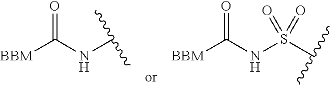

on one or more of Ring X, Ring Z, or R_{z}.

In certain embodiments, the present invention provides a compound of formula I-aa-1, wherein R¹ is

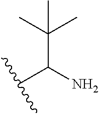 [SMILES: *C(N)C(C)(C)C]

(where one of the hydrogen atoms of the NH₂ group is replaced with -L-) and s is 1 as shown, to provide a compound of formula I-aa-11:

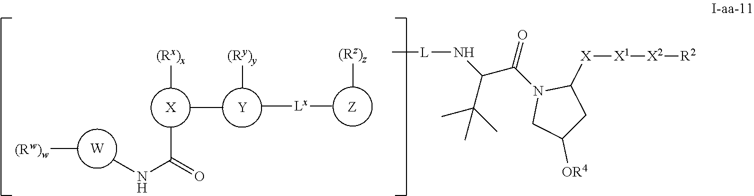

or a pharmaceutically acceptable salt thereof, wherein
Ring Z is a ring selected from phenyl, naphthyl, a 5-10 membered heteroaryl containing 1-4 heteroatoms independently selected from nitrogen, oxygen, selenium, and sulfur, and a 5-9 membered saturated or partially unsaturated monocyclic, bicyclic, bridged bicylic, or spirocyclic carbocyclyl or heterocyclyl with 1-4 heteroatoms independently selected from nitrogen, oxygen, and sulfur; and
wherein each of L, L^{x}, X, X¹, X², R², R⁴, R^{w}, R^{x}, R^{y}, R^{z}, Ring W, Ring X, Ring Y, v, w, x, y, and z is as defined above and described in embodiments herein, both singly and in combination.

In certain embodiments, the present invention provides a compound of formula I-aa-1, wherein

R¹ is

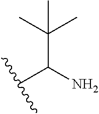 [SMILES: *C(N)C(C)(C)C]

(where one of the hydrogen atoms of the NH₂ group is replaced with -L-), R₂ is

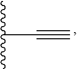 [SMILES: *C#C]

and s is 1 as shown, to provide a compound of formula I-aa-12:

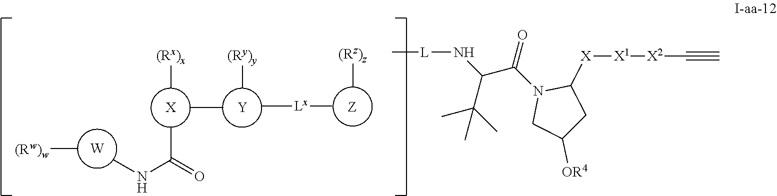

or a pharmaceutically acceptable salt thereof, wherein
Ring Z is a ring selected from phenyl, naphthyl, a 5-10 membered heteroaryl containing 1-4 heteroatoms independently selected from nitrogen, oxygen, selenium, and sulfur, and a 5-9 membered saturated or partially unsaturated monocyclic, bicyclic, bridged bicylic, or spirocyclic carbocyclyl or heterocyclyl with 1-4 heteroatoms independently selected from nitrogen, oxygen, and sulfur; and
wherein each of L, L^{x}, X, X¹, X², R⁴, R^{w}, R^{x}, R^{y}, R^{z}, Ring W, Ring X, Ring Y, v, w, x, y, and z is as defined above and described in embodiments herein, both singly and in combination.

In certain embodiments, the present invention provides a compound of formula I-aa-1, wherein R¹ is

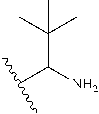 [SMILES: *C(N)C(C)(C)C]

(where one of the hydrogen atoms of the NH₂ group is replaced with -L-), s is 1, and Ring Z is cyclohexyl as shown, to provide a compound of formula I-aa-13:

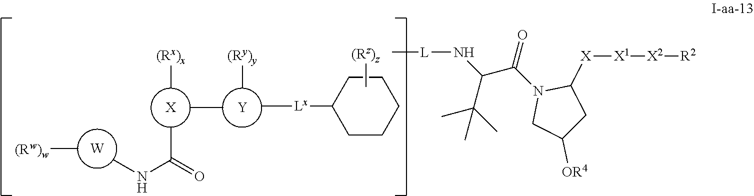

or a pharmaceutically acceptable salt thereof, wherein each of L, L^{x}, X, X¹, X², R², R⁴, R^{w}, R^{x}, R^{y}, R^{z}, Ring W, Ring X, Ring Y, v, w, x, y, and z is as defined above and described in embodiments herein, both singly and in combination.

In certain embodiments, the present invention provides a compound of formula I-aa-1, wherein R¹ is

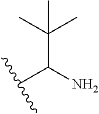 [SMILES: *C(N)C(C)(C)C]

(where one of the hydrogen atoms of the NH₂ group is replaced with -L-), s is 1, and Ring Z is cyclohexyl as shown, to provide a compound of formula I-aa-14:

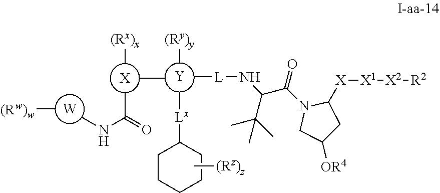

or a pharmaceutically acceptable salt thereof, wherein each of L, L^{x}, X, X¹, X², R², R⁴, R^{w}, R^{x}, R^{y}, R^{z}, Ring W, Ring X, Ring Y, v, w, x, y, and z is as defined above and described in embodiments herein, both singly and in combination.

In certain embodiments, the present invention provides a compound of formula I-aa-1, wherein

R¹ is

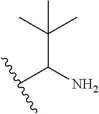 [SMILES: *C(N)C(C)(C)C]

(where one of the hydrogen atoms of the NH₂ group is replaced with -L-), R₂ is

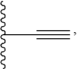 [SMILES: *C#C]

is 1, and Ring Z is cyclohexyl as shown, to provide a compound of formula I-aa-15:

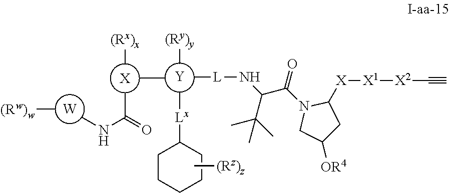

or a pharmaceutically acceptable salt thereof, wherein each of L, L^{x}, X, X¹, X², R⁴, R^{w}, R^{x}, R^{y}, R^{z}, Ring W, Ring X, Ring Y, v, w, x, y, and z is as defined above and described in embodiments herein, both singly and in combination.

In certain embodiments, the present invention provides a compound of formula I-aa-1, wherein

R¹ is

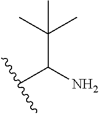 [SMILES: *C(N)C(C)(C)C]

(where one of the hydrogen atoms of the NH₂ group is replaced with -L-), Ring X is 1,2,3,4-tetrahydroisoquinolinylenyl, Ring W is benzothiazolyl, and s is I as shown, to provide a compound of formula I-aa-16:

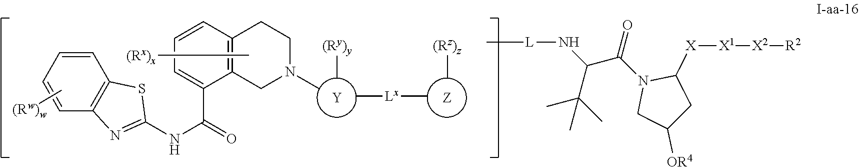

or a pharmaceutically acceptable salt thereof, wherein
Ring Z is a ring selected from phenyl, naphthyl, a 5-10 membered heteroaryl containing 1-4 heteroatoms independently selected from nitrogen, oxygen, selenium, and sulfur, and a 5-9 membered saturated or partially unsaturated monocyclic, bicyclic, bridged bicylic, or spirocyclic carbocyclyl or heterocyclyl with 1-4 heteroatoms independently selected from nitrogen, oxygen, and sulfur; and
wherein each of L, L^{x}, X, X¹, X², R², R⁴, R^{w}, R^{x}, R^{y}, R^{z}, Ring Y, v, w, x, y, and z is as defined above and described in embodiments herein, both singly and in combination.

In certain embodiments, the present invention provides a compound of formula I-aa-1, wherein R¹ is

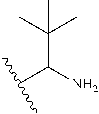 [SMILES: *C(N)C(C)(C)C]

(where one of the hydrogen atoms of the NH₂ group is replaced with -L-), Ring X is 1,2,3,4-tetrahydroisoquinolinylenyl, Ring W is benzothiazolyl, s is 1, and Ring Z is cyclohexyl as shown, to provide a compound of formula I-aa-17:

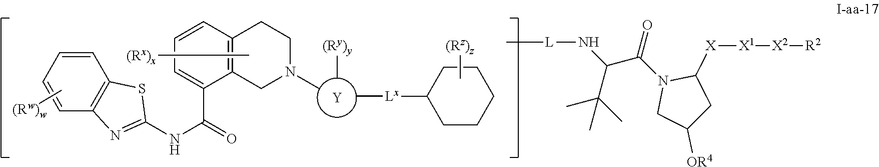

or a pharmaceutically acceptable salt thereof, wherein each of L, L^{x}, X, X¹, X², R², R⁴, R^{w}, R^{x}, R^{y}, R^{z}, Ring W, v, w, x, y, and z is as defined above and described in embodiments herein, both singly and in combination.

In certain embodiments, the present invention provides a compound of formula I-aa-1, wherein R¹ is

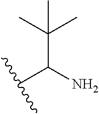 [SMILES: *C(N)C(C)(C)C]

(where one of the hydrogen atoms of the NH₂ group is replaced with -L-), Ring X is 1,2,3,4-tetrahydroisoquinolinylenyl, Ring W is benzothiazolyl, s is 1, and Ring Z is cyclohexyl as shown, to provide a compound of formula I-aa-18:

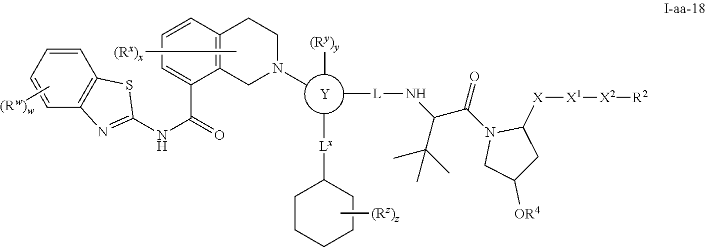

or a pharmaceutically acceptable salt thereof, wherein each of L, L^{x}, X, X¹, X², R², R⁴, R^{w}, R^{x}, R^{y}, R^{z}, Ring Y, v, w, x, y, and z is as defined above and described in embodiments herein, both singly and in combination.

In certain embodiments, the present invention provides a compound of formula I-aa-1, wherein R¹ is

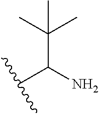 [SMILES: *C(N)C(C)(C)C]

(where one of the hydrogen atoms of the NH₂ group is replaced with -L-), Ring X is 1,2,3,4-tetrahydroisoquinolinylenyl, Ring W is benzothiazolyl, R₂ is

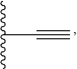 [SMILES: *C#C]

s is 1, and Ring Z is cyclohexyl as shown, to provide a compound of formula I-aa-19:

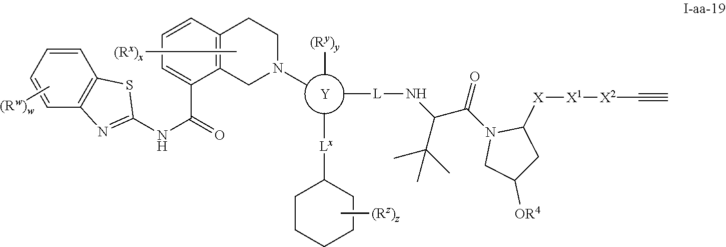

or a pharmaceutically acceptable salt thereof, wherein each of L, L^{x}, X, X¹, X², R⁴, R^{w}, R^{x}, R^{y}, R^{z}, Ring Y, v, w, x, y, and z is as defined above and described in embodiments herein, both singly and in combination.

In certain embodiments, the present invention provides a compound of formula I-bb-1:

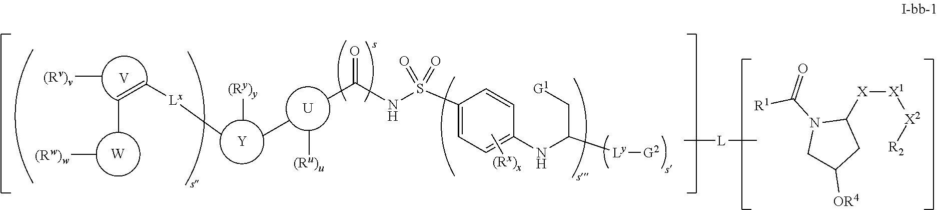

or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
Ring V is a bivalent ring selected from a 5-6 membered partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Rᵤ, Rᵥ, R^{w}, R^{x1}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G¹ is —S-aryl, —S-heteroaryl, or —R^{A};
G² is hydrogen or

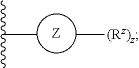

Ring Z is a ring selected from phenyl, naphthyl, a 5-10 membered heteroaryl containing 1-4 heteroatoms independently selected from nitrogen, oxygen, and sulfur, and a 4-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
L^{x} and L^{y} are, independently, a covalent bond or a bivalent, saturated or partially unsaturated, straight or branched C₁-s hydrocarbon chain, wherein 0-3 methylene units of L^{x} are independently replaced by optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
S, s″, and s″ are, independently, 0 or 1;
s′ is 1 or 2;
u, v, x, w, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

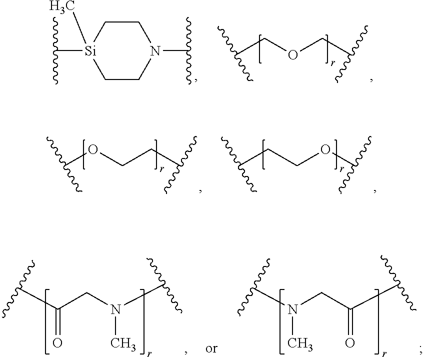

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R₂ is hydrogen, halogen, —CN,

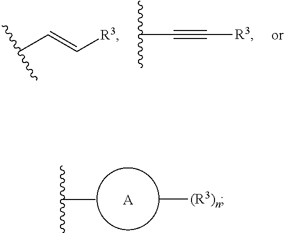

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R₃ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R) OR, —C(R)₂NRC(O)R, —C(R) ¿NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R: or
   two R₃ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃P(O)R₂, —(CR₂)₁₋₃P(O)(OR)₂, —(CR₂)₁₋₃OP(O)R₂, —(CR₂)₁₋₃OP(O)(OR)₂, —C(O)(CR₂)₁₋₃P(O)R₂, —C(O)(CR₂)₁₋₃P(O)(OR)₂, —C(O)(CR₂)₁₋₃OP(O)R₂, —C(O)(CR₂)₁₋₃OP(O)(OR)₂, or R^{A};
n is 0, 1, 2, 4, or 5.

In certain embodiments, the present invention provides a compound of formula I-bb-1, wherein R¹ is

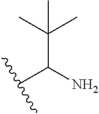 [SMILES: *C(N)C(C)(C)C]

(where one of the hydrogen atoms of the NH₂ group is replaced with -L-), s″ and s″ are 1, and G¹ is —SPh as shown, to provide a compound of formula I-bb-2:

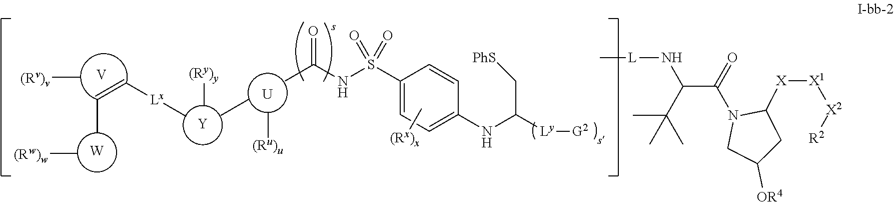

or a pharmaceutically acceptable salt thereof, wherein each of L, L^{x}, L^{y}, X, X¹, X², R², R⁴, R^{u}, R^{v}, R^{w}, R^{x}, R^{y}, Ring U, Ring V, Ring W, Ring Y, G², s, s′, u, v, w, x, and y is as defined above and described in embodiments herein, both singly and in combination.

In certain embodiments, the present invention provides a compound of formula I-bb-1, wherein R¹ is

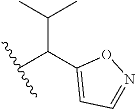 [SMILES: *C(c1ccno1)C(C)C]

(where one of the hydrogen atoms of the isoxazolyl group is replaced with -L-), s″ and s′″ are 1, and G¹ is —SPh as shown, to provide a compound of formula I-bb-3:

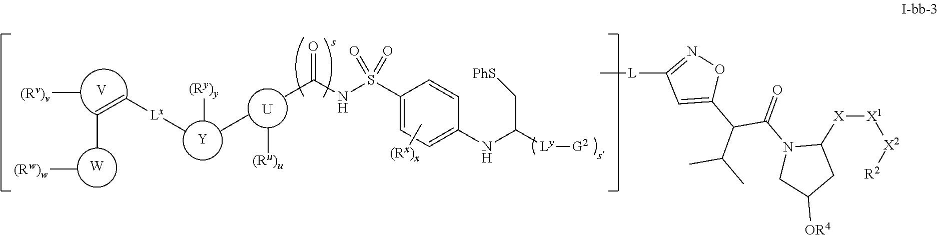

or a pharmaceutically acceptable salt thereof, wherein each of L, L^{x}, L^{y}, X^{z}, X¹, X², R², R⁴, R^{u}, R^{v}, R^{w}, R^{x}, R^{y}, Ring U, Ring V, Ring W, Ring Y, G², s, s′, u, v, w, x, and y is as defined above and described in embodiments herein, both singly and in combination.

In certain embodiments, the present invention provides a compound of formula I-bb-1, wherein R¹ is

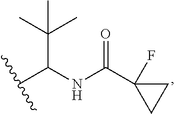 [SMILES: *C(NC(=O)C1(F)CC1)C(C)(C)C]

X² is phenylenyl, s″ and s′″ are 1, and G¹ is —SPh as shown, to provide a compound of formula I-bb-4:

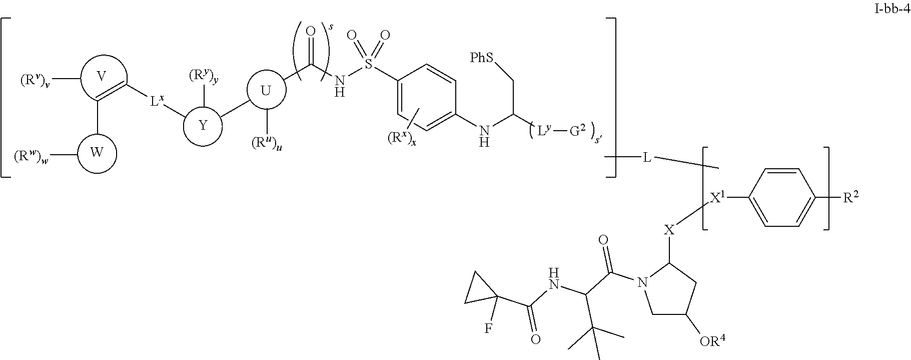

or a pharmaceutically acceptable salt thereof, wherein each of L, L^{x}, L^{y}, X, X¹, R², R⁴, R^{u}, R^{v}, R^{w}, R^{x}, R^{y}, Ring U, Ring V, Ring W, Ring Y, G², s, s′, u, v, w, x, and y is as defined above and described in embodiments herein, both singly and in combination.

In certain embodiments, the present invention provides a compound of formula I-bb-1, wherein R¹ is

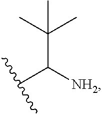 [SMILES: *C(N)C(C)(C)C]

Ring V is cyclohexenyl, Ring U and X² are phenylenyl, Ring W is phenyl, s″ and s′″ are 1, and G¹ is —SPh as shown, to provide a compound of formula I-bb-5:

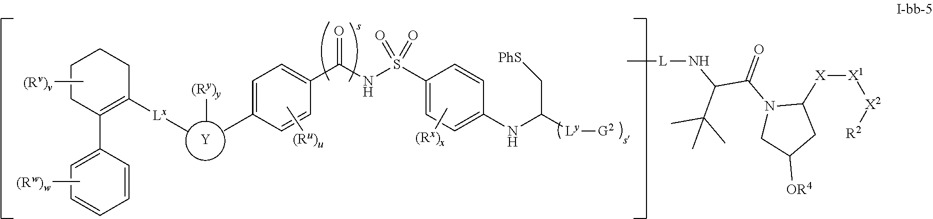

or a pharmaceutically acceptable salt thereof, wherein each of L, L^{x}, L^{y}, X, X¹, X², R², R⁴, R^{u}, R^{v}, R^{w}, R^{x}, R^{y}, Ring Y, G², s, s′, u, v, w, x, and y is as defined above and described in embodiments herein, both singly and in combination.

In certain embodiments, the present invention provides a compound of formula I-bb-1, wherein R¹ is

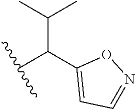 [SMILES: *C(c1ccno1)C(C)C]

(where one of the hydrogen atoms of the isoxazolyl group is replaced with -L-), Ring V is cyclohexenyl, Ring U and X² are phenylenyl, Ring W is phenyl, s″ and s′″ are 1, and G¹ is —SPh as shown, to provide a compound of formula I-bb-6:

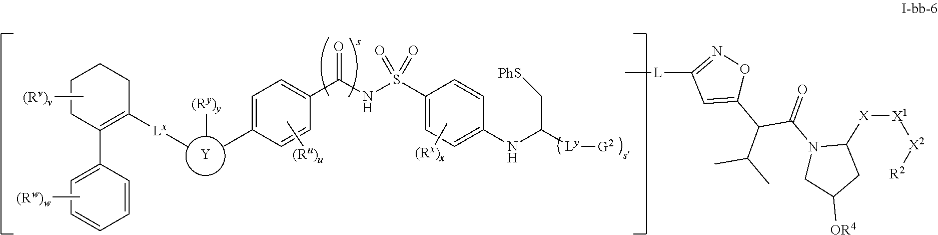

or a pharmaceutically acceptable salt thereof, wherein each of L, L^{x}, L^{y}, X, X¹, X², R², R⁴, R^{u}, R^{v}, R^{w}, R^{x}, R^{y}, Ring Y, G², s, s′, u, v, w, x, and y is as defined above and described in embodiments herein, both singly and in combination.

In certain embodiments, the present invention provides a compound of formula I-bb-1, wherein R¹ is

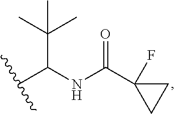 [SMILES: *C(NC(=O)C1(F)CC1)C(C)(C)C]

Ring V is cyclohexenyl, Ring U and X² are phenylenyl, Ring W is phenyl, s″ and s′″ are 1, and G¹ is —SPh as shown, to provide a compound of formula I-bb-7:

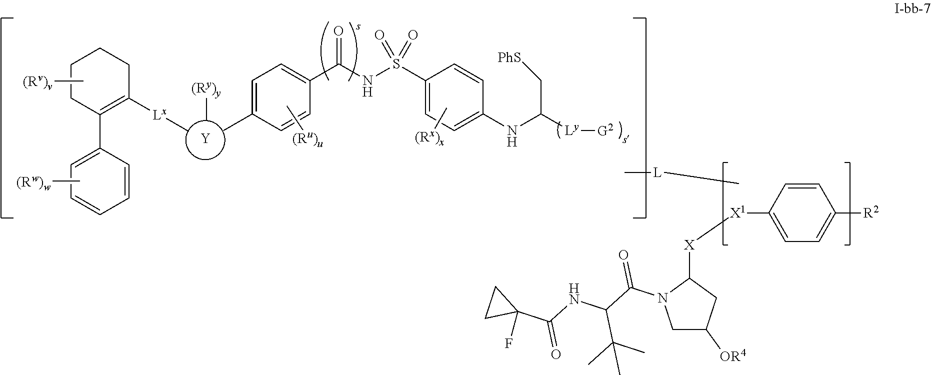

or a pharmaceutically acceptable salt thereof, wherein each of L, L^{x}, L^{y}, X, X¹, R², R₄, R^{u}, R^{v}, R^{w}, R^{x}, R^{y}, Ring Y, G², s, s′, u, v, w, x, and y is as defined above and described in embodiments herein, both singly and in combination.

In certain embodiments, the present invention provides a compound of formula I-cc-1:

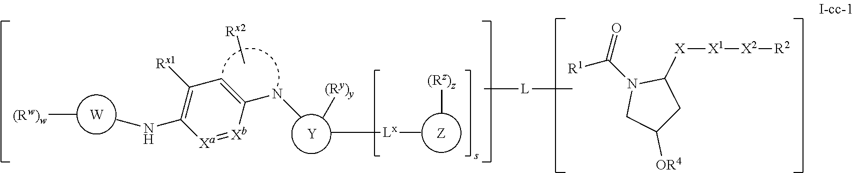

or a pharmaceutically acceptable salt, wherein:
Ring W and Ring Z are, independently, a ring selected from phenyl, naphthyl, a 5-10 membered heteroaryl containing 1-4 heteroatoms independently selected from nitrogen, oxygen, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;

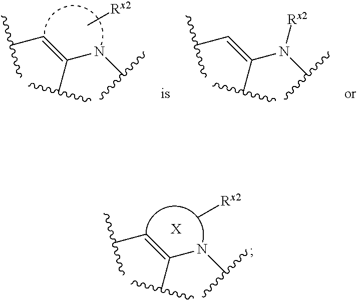

Ring X is a fused ring selected from a 5-6 membered saturated or partially unsaturated heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;

R^{w}, R^{x1}, R^{x2}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a bivalent, saturated or partially unsaturated, straight or branched C₁₋₅ hydrocarbon chain, wherein 0-3 methylene units of L¹ are independently replaced by optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
X^{a} and X^{b} are, independently, a carbon atom or a nitrogen atom;
s is 0 or 1;
w, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O); N(R)—, —N(R)C(O)—, —C(O)N(R)—, —OC(O)N(R)—, —N(R)C(O)O—,

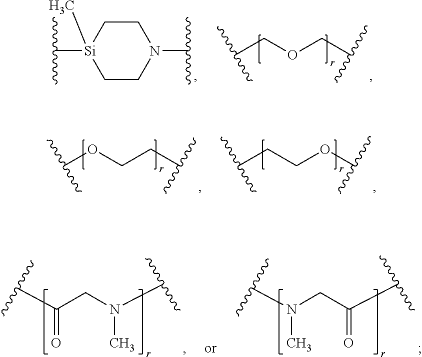

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R₂ is hydrogen, halogen, —CN,

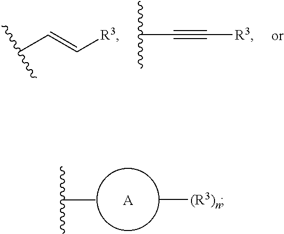

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R₃ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R) OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R: or two R₃ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃P(O)R₂, —(CR₂)₁₋₃P(O)(OR)₂, —(CR₂)₁₋₃OP(O)R₂, —(CR₂)₁₋₃OP(O)(OR)₂, —C(O)(CR₂)₁₋₃P(O)R₂, —C(O)(CR₂)₁₋₃P(O)(OR)₂, —C(O)(CR₂)₁₋₃OP(O)R₂, —C(O)(CR₂)₁₋₃OP(O)(OR)₂, or R^{A};
n is 0, 1, 2, 4, or 5.

In certain embodiments, the present invention provides a compound of formula I-cc-1, wherein R¹ is

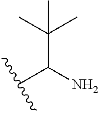 [SMILES: *C(N)C(C)(C)C]

(where one of the H groups of the NH₂ group is replaced with -L-) as shown, to provide a compound of formula I-cc-2:

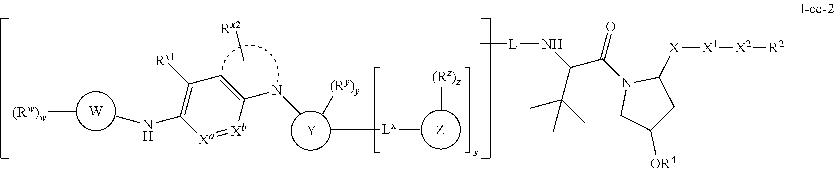

or a pharmaceutically acceptable salt thereof, wherein each of L, L^{x}, X, X¹, X², X^{a}, X^{b}, R₂, R₄, R^{w}, R^{x1}, R^{x2}, R^{y}, R^{z},

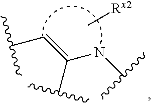

Ring W, Ring Y, Ring Z, s, w, y, and z is as defined above and described in embodiments herein, both singly and in combination.

In certain embodiments, the present invention provides a compound of formula I-cc-1, wherein R¹ is

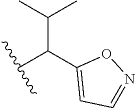 [SMILES: *C(c1ccno1)C(C)C]

(where one of the H groups of the isoxazolyl group is replaced with -L-) as shown, to provide a compound of formula I-cc-3:

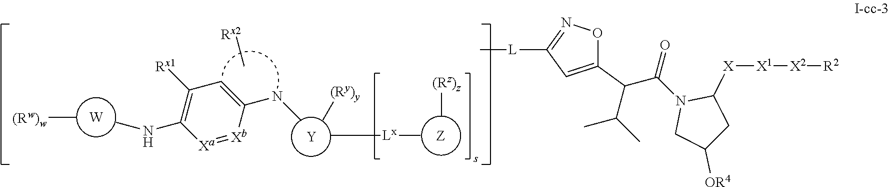

or a pharmaceutically acceptable salt thereof, wherein each of L, L^{x}, X, X¹, X², X^{a}, X^{b}, R₂, R₄, R_{w}, R^{x1}, R^{x2}, R^{y}, R²,

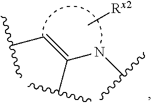

Ring W, Ring Y, Ring Z, s, w, y, and z is as defined above and described in embodiments herein, both singly and in combination.

In certain embodiments, the present invention provides a compound of formula I-cc-1, wherein R¹ is

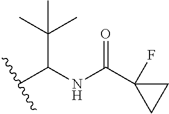 [SMILES: *C(NC(=O)C1(F)CC1)C(C)(C)C]

and X² is phenylenyl as shown, to provide a compound of formula I-cc-4:

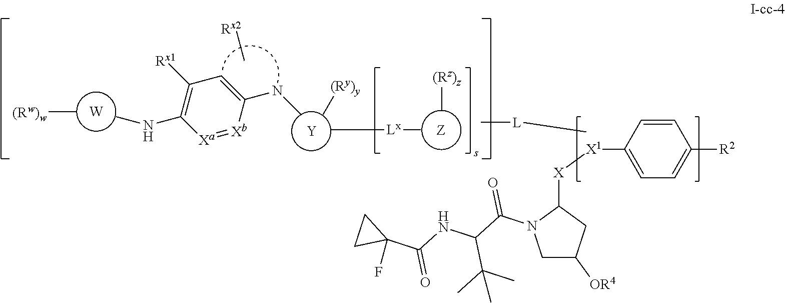

or a pharmaceutically acceptable salt thereof, wherein each of L, LA, X, X¹, X^{a}, X^{b}, R₂, R₄, R^{w}, R^{x1}, R^{x2}, R^{y}, R^{z},

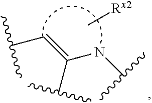

Ring W, Ring Y, Ring Z, s, w, y, and z is as defined above and described in embodiments herein, both singly and in combination.

In certain embodiments, the present invention provides a compound of formula I-cc-1, wherein R¹ is

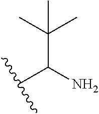 [SMILES: *C(N)C(C)(C)C]

(where one of the H groups of the NH₂ group is replaced with -L-), Ring W is benzothiazolyl, and X^{a} and X^{b} are nitrogen as shown, to provide a compound of formula I-cc-5:

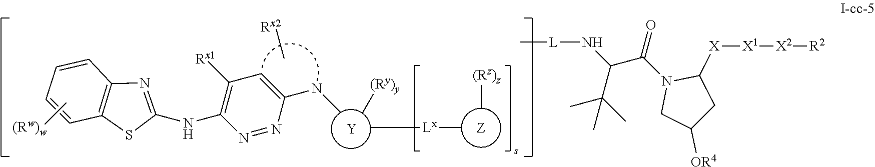

or a pharmaceutically acceptable salt thereof, wherein each of L, L^{x}, X, X¹, X², R², R⁴, R^{w}, R^{x1}, R^{x2}, R^{y}, R^{z},

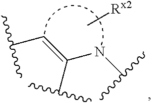

Ring Y, Ring Z, s, w, y, and z is as defined above and described in embodiments herein, both singly and in combination.

In certain embodiments, the present invention provides a compound of formula I-cc-1, wherein R¹ is

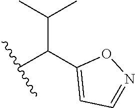 [SMILES: *C(c1ccno1)C(C)C]

(where one of the H groups of the isoxazolyl group is replaced with -L-), Ring W is benzothiazolyl, and X^{a} and X^{b} are nitrogen as shown, to provide a compound of formula I-cc-6:

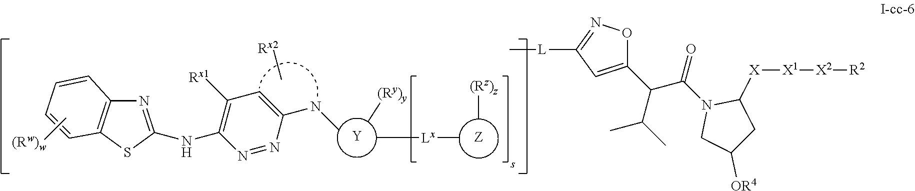

or a pharmaceutically acceptable salt thereof, wherein each of L, L^{x}, X, X¹, X², R², R⁴, R^{w}, R^{x1}, R^{x2}, R^{y}, R^{z},

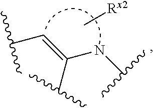

Ring Y, Ring Z, s, w, y, and z is as defined above and described in embodiments herein, both singly and in combination.

In certain embodiments, the present invention provides a compound of formula I-cc-1, wherein R¹ is

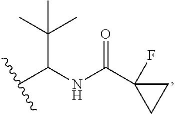 [SMILES: *C(NC(=O)C1(F)CC1)C(C)(C)C]

X² is phenylenyl, Ring W is benzothiazolyl, and X^{a} and X^{b} are nitrogen as shown, to provide a compound of formula I-cc-7:

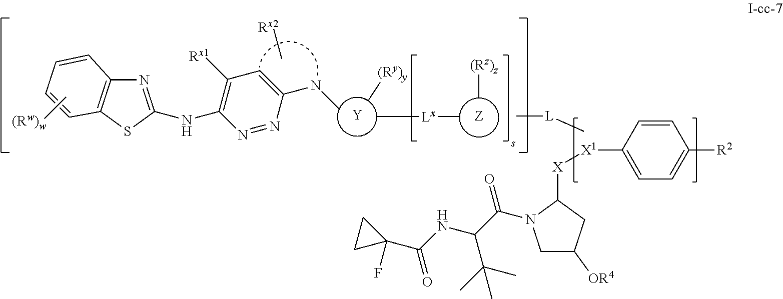

or a pharmaceutically acceptable salt thereof, wherein each of L, L^{x}, X, X¹, R², R₄, R^{w}, R^{x1}, R^{x2}, R^{y}, R^{z},

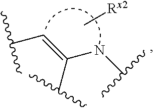

Ring Y, Ring Z, s, w, y, and z is as defined above and described in embodiments herein, both singly and in combination.

In certain embodiments, the present invention provides a compound of formula I-cc-1, wherein R¹ is

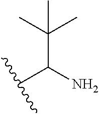 [SMILES: *C(N)C(C)(C)C]

(where one of the H groups of the NH₂ group is replaced with -L-), R₂ is

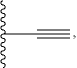 [SMILES: *C#C]

and s is 1, as shown, to provide a compound of formula I-cc-8:

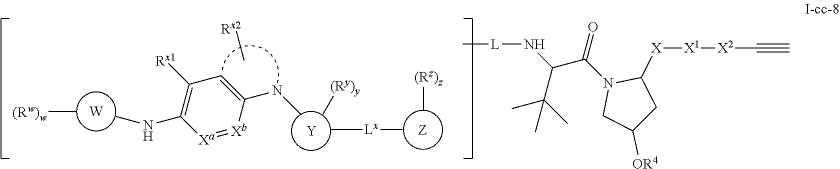

or a pharmaceutically acceptable salt thereof, wherein each of L, L^{x}, X, X¹, X², X^{a}, X^{b}, R₄, R^{w}, R^{x1}, R^{x2}, R^{y}, R₂,

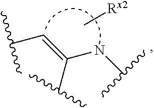

Ring W, Ring Y, Ring Z, w, y, and z is as defined above and described in embodiments herein, both singly and in combination.

In certain embodiments, the present invention provides a compound of formula I-cc-1, wherein R¹ is

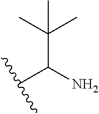 [SMILES: *C(N)C(C)(C)C]

(where one of the H groups of the NH₂ group is replaced with -L-), R₂ is

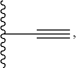 [SMILES: *C#C]

s is 1,

Ring W is benzothiazolyl, and X^{a} and X′ are nitrogen as shown, to provide a compound of formula I-cc-9:

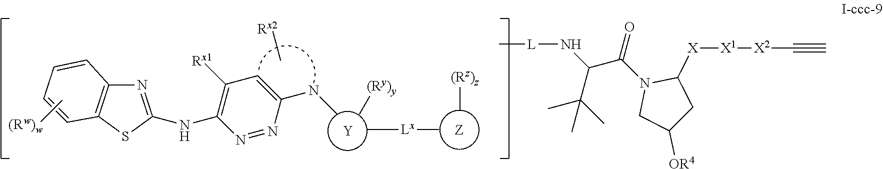

or a pharmaceutically acceptable salt thereof, wherein each of L, L^{x}, X, X¹, X², R⁴, R^{w}, R^{x1}, R^{x2}, R^{y}, R²,

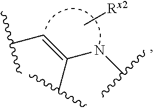

Ring Y, Ring Z, w, y, and z is as defined above and described in embodiments herein, both singly and in combination.

In certain embodiments, the present invention provides a compound of formula I-cc-1, wherein R¹ is

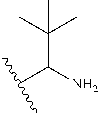 [SMILES: *C(N)C(C)(C)C]

(where one of the H groups of the NH₂ group is replaced with -L-), R₂ is

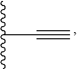 [SMILES: *C#C]

and s is 1, as shown, to provide a compound of formula I-cc-10:

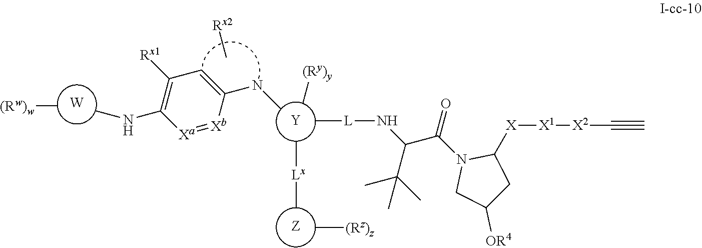

or a pharmaceutically acceptable salt thereof, wherein each of L, L^{x}, X, X¹, X², X^{a}, X^{b}, R₄, R^{w}, R^{x1}, R^{x2}, R^{y}, R_{z},

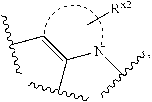

Ring W, Ring Y, Ring Z, w, y, and z is as defined above and described in embodiments herein, both singly and in combination.

In certain embodiments, the present invention provides a compound of formula I-cc-1, wherein R¹ is

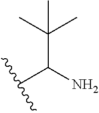 [SMILES: *C(N)C(C)(C)C]

(where one of the H groups of the NH₂ group is replaced with -L-), R₂ is

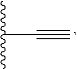 [SMILES: *C#C]

s is 1,

Ring W is benzothiazolyl, and X^{a} and X^{b} are nitrogen as shown, to provide a compound of formula I-cc-11:

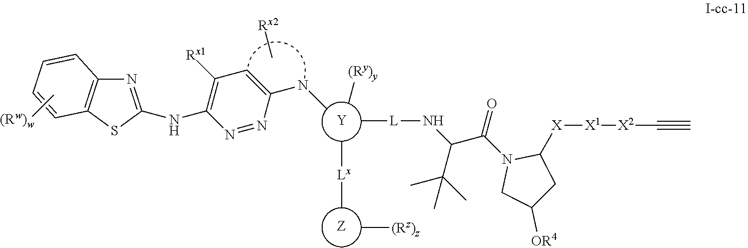

or a pharmaceutically acceptable salt thereof, wherein each of L, L^{x}, X, X¹, X², R⁴, R_{w}, R^{x1}, R^{x2}, R^{y}, R^{z},

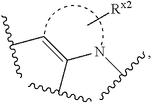

Ring Y, Ring Z, w, y, and z is as defined above and described in embodiments herein, both singly and in combination.

In certain embodiments, the present invention provides a compound of formula I-cc-1, wherein R¹ is

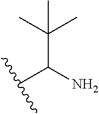 [SMILES: *C(N)C(C)(C)C]

(where one of the H groups of the NH₂ group is replaced with -L-), R₂ is

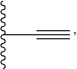 [SMILES: *C#C]

s is 1, Ring W is benzothiazolyl,

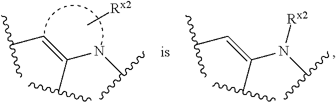

and X^{a} and X^{b} are nitrogen as shown, to provide a compound of formula I-cc-12:

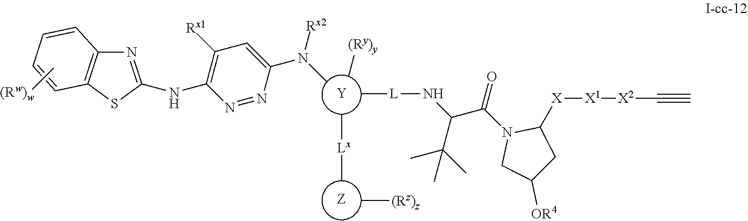

or a pharmaceutically acceptable salt thereof, wherein each of L, L^{x}, X, X¹, X², R⁴, R^{w}, R^{x1}, R^{x2}, R^{y}, R^{z}, Ring Y, Ring Z, w, y, and z is as defined above and described in embodiments herein, both singly and in combination.

In certain embodiments, the present invention provides a compound of formula I-cc-1, wherein R¹ is

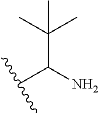 [SMILES: *C(N)C(C)(C)C]

(where one of the H groups of the NH₂ group is replaced with -L-), R₂ is

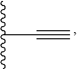 [SMILES: *C#C]

s is 1, Ring W is benzothiazolyl,

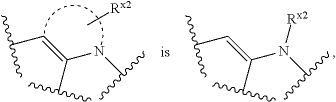

### Ring Z is

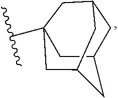 [SMILES: *C12CC3CC(CC(C3)C1)C2]

z is 0, and X^{a} and X^{b} are nitrogen as shown, to provide a compound of formula I-cc-13:

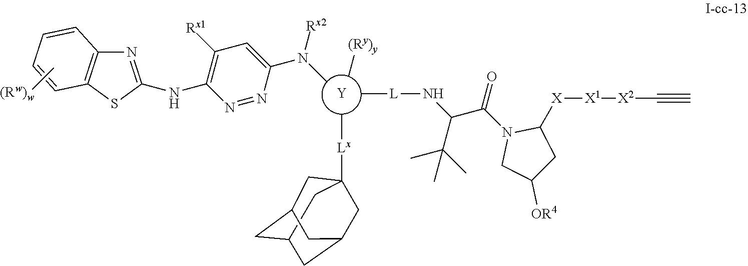

or a pharmaceutically acceptable salt thereof, wherein each of L, L^{x}, X, X¹, X², R⁴, R^{w}, R^{x1}, R^{x2}, R^{y}, Ring Y, w, and y is as defined above and described in embodiments herein, both singly and in combination.

In certain embodiments, the present invention provides a compound of formula I-cc-1, wherein R¹ is

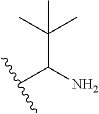 [SMILES: *C(N)C(C)(C)C]

(where one of the H groups of the NH₂ group is replaced with -L-), R₂ is

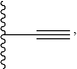 [SMILES: *C#C]

s is 1, Ring W is benzothiazolyl,

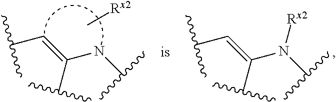

L is —CONHSO₂—(CH₂)₁₋₁₀—CO—, and X^{a} and X^{b} are nitrogen as shown, to provide a compound of formula I-cc-14:

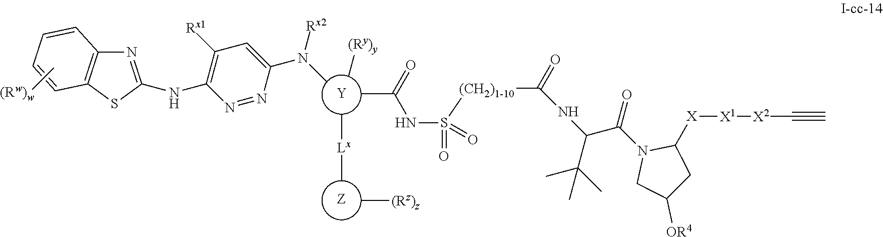

or a pharmaceutically acceptable salt thereof, wherein each of LA, X, X¹, X², R⁴, R^{w}, Rₓ₁, R^{x2}, R^{y}, R^{z}, Ring Y, Ring Z, w, y, and z is as defined above and described in embodiments herein, both singly and in combination.

In certain embodiments, the present invention provides a compound of formula I-dd-1:

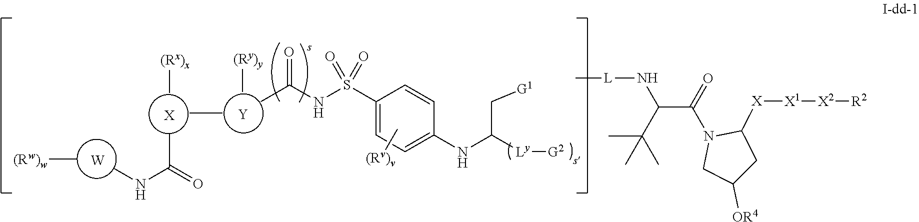

or a pharmaceutically acceptable salt, wherein:
Ring W is a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur; Ring X is a bicyclic ring selected from a 9-11 membered partially unsaturated heterocyclylenyl with 1-4 heteroatoms independently selected from nitrogen, oxygen, and sulfur, and a 9-10 membered heteroarylenyl containing 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{v}, R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R—S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{y} is a covalent bond or a bivalent, saturated or partially unsaturated, straight or branched C₁₋₅ hydrocarbon chain, wherein 0-3 methylene units of L¹ are independently replaced by a optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
G¹ is —S-aryl, —S-heteroaryl, or —R^{A};
G² is hydrogen or

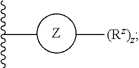

Ring Z is a ring selected from phenyl, naphthyl, a 5-10 membered heteroaryl containing 1-4 heteroatoms independently selected from nitrogen, oxygen, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
s is 0 or 1;
s′ is 1 or 2;
v, w, x, y, and z are, independently, 0, 1, 2, 3, or 4; L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

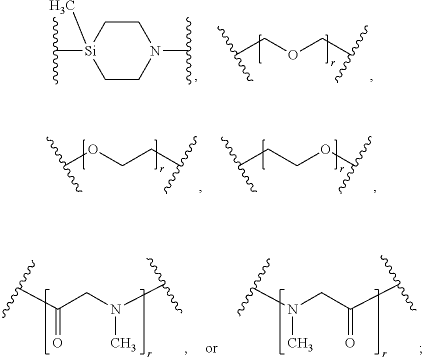

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R) ¿R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R₂ is hydrogen, halogen, —CN,

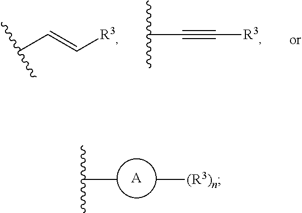

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R₃ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R) OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R; or two R₃ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃P(O)R₂, —(CR₂)₁₋₃P(O)(OR)₂, —(CR₂)₁₋₃OP(O)R₂, —(CR₂)₁₋₃OP(O)(OR)₂, —C(O)(CR₂)₁₋₃P(O)R₂, —C(O)(CR₂)₁₋₃P(O)(OR)₂, —C(O)(CR₂)₁₋₃OP(O)R₂, —C(O)(CR₂)₁₋₃OP(O)(OR)₂, or R^{A};
n is 0, 1, 2, 4, or 5.

In certain embodiments, the present invention provides a compound of formula I-dd-1, wherein

R¹ is

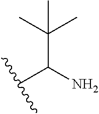 [SMILES: *C(N)C(C)(C)C]

(where one of the hydrogen atoms of the NH₂ group is replaced with -L-) and G′ is —SPh as shown, to provide a compound of formula I-dd-2:

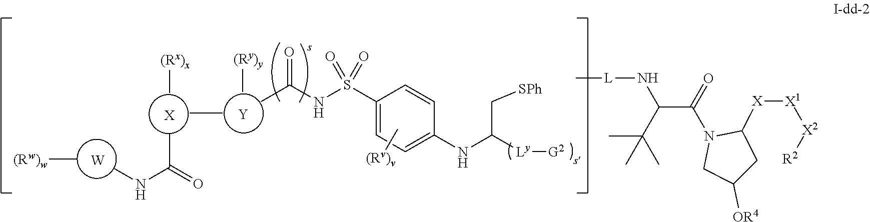

or a pharmaceutically acceptable salt thereof, wherein each of L, L^{y}, X, X¹, X², R², R⁴, R^{v}, R^{w}, R^{x}, R^{y}, Ring W, Ring X, Ring Y, G², s, s′, v, w, x, and y is as defined above and described in embodiments herein, both singly and in combination.

In certain embodiments, the present invention provides a compound of formula I-dd-1, wherein R¹ is

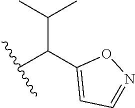 [SMILES: *C(c1ccno1)C(C)C]

(where one of the hydrogen atoms of the isoxazolyl group is replaced with -L-) and G′ is —SPh as shown, to provide a compound of formula I-dd-3:

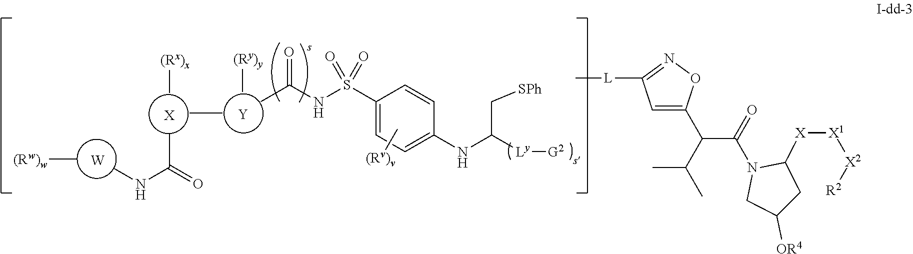

or a pharmaceutically acceptable salt thereof, wherein each of L, L^{y}, X, X¹, X², R², R⁴, R^{v}, R^{w}, R^{x}, R^{y}, Ring W, Ring X, Ring Y, G², s, s′, v, w, x, and y is as defined above and described in embodiments herein, both singly and in combination.

In certain embodiments, the present invention provides a compound of formula I-dd-1, wherein

R¹ is

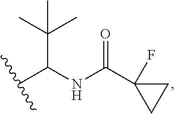 [SMILES: *C(NC(=O)C1(F)CC1)C(C)(C)C]

X² is phenylenyl, and G′ is —SPh as shown, to provide a compound of formula I-dd-4:

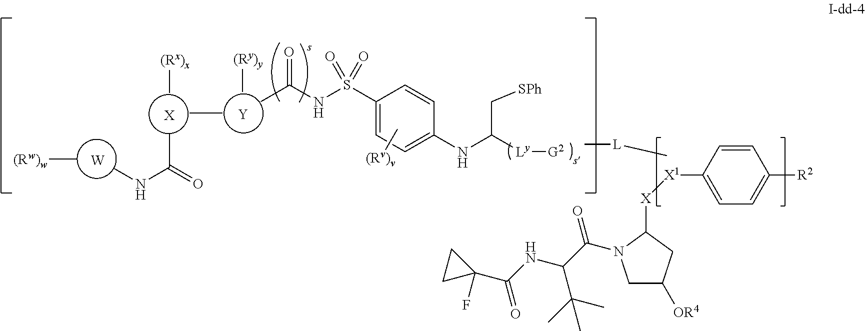

or a pharmaceutically acceptable salt thereof, wherein each of L, L^{y}, X, X¹, R², R₄, R^{v}, R^{w}, R^{x}, R^{y}, Ring W, Ring X, Ring Y, G², s, s′, v, w, x, and y is as defined above and described in embodiments herein, both singly and in combination.

In certain embodiments, the present invention provides a compound of formula I-dd-1, wherein R¹ is

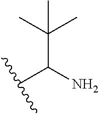 [SMILES: *C(N)C(C)(C)C]

(where one of the hydrogen atoms of the NH₂ group is replaced with -L-), Ring X is 1,2,3,4-tetrahydroisoquinolinylenyl, and Ring W is benzothiazolyl, and is —SPh as shown, to provide a compound of formula I-dd-5:

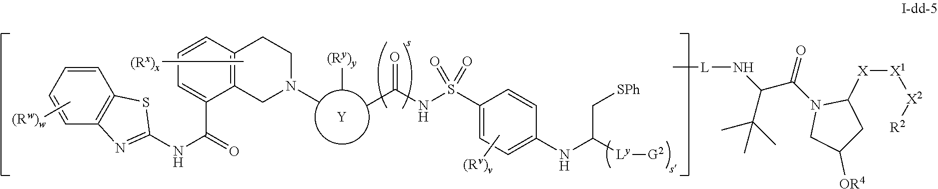

or a pharmaceutically acceptable salt thereof, wherein each of L, L^{y}, X, X¹, X², R², R⁴, R^{v}, R^{w}, R^{x}, R^{y}, Ring Y, G², s, s′, v, w, x, and y is as defined above and described in embodiments herein, both singly and in combination.

In certain embodiments, the present invention provides a compound of formula I-dd-1, wherein R¹ is

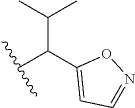 [SMILES: *C(c1ccno1)C(C)C]

(where one of the hydrogen atoms of the isoxazolyl group is replaced with -L-), Ring X is 1,2,3,4-tetrahydroisoquinolinylenyl, and Ring W is benzothiazolyl, and G¹ is —SPh as shown, to provide a compound of formula I-dd-6:

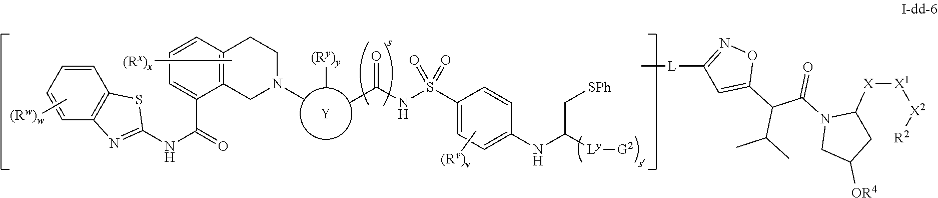

or a pharmaceutically acceptable salt thereof, wherein each of L, L^{y}, X, X¹, X², R², R⁴, R^{v}, R^{w}, R^{x}, R^{y}, Ring Y, G², s, s′, v, w, x, and y is as defined above and described in embodiments herein, both singly and in combination.

In certain embodiments, the present invention provides a compound of formula I-dd-1, wherein R¹ is

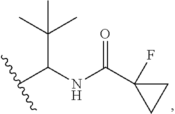 [SMILES: *C(NC(=O)C1(F)CC1)C(C)(C)C]

X² is phenylenyl, Ring X is 1,2,3,4-tetrahydroisoquinolinylenyl, and Ring W is benzothiazolyl, and G¹ is —SPh as shown, to provide a compound of formula I-dd-7:

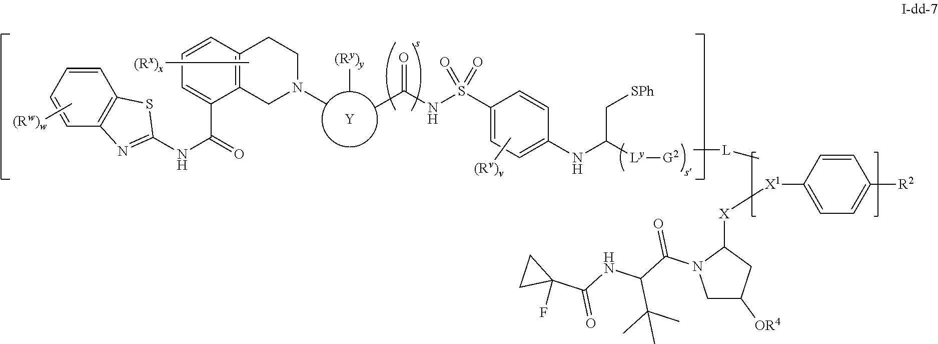

or a pharmaceutically acceptable salt thereof, wherein each of L, L^{y}, X, X¹, R², R⁴, R^{v}, R^{w}, R^{x}, R^{y}, Ring Y, G², s, s′, v, w, x, and y is as defined above and described in embodiments herein, both singly and in combination.

In some embodiments, LBM is

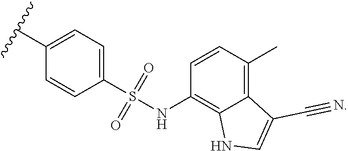 [SMILES: *c1ccc(S(=O)(=O)Nc2ccc(C)c3c(C#N)c[nH]c23)cc1]

In some embodiments, LBM is

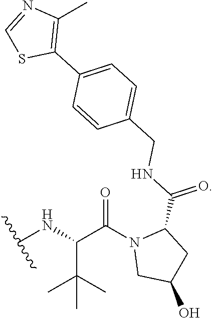 [SMILES: *N[C@H](C(=O)N1C[C@H](O)C[C@H]1C(=O)NCc1ccc(-c2scnc2C)cc1)C(C)(C)C]

In some embodiments, LBM is

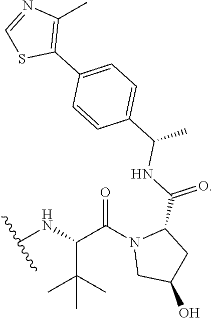 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

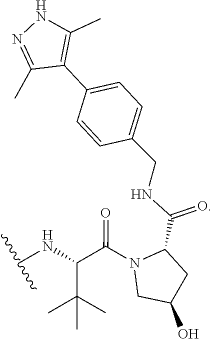 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

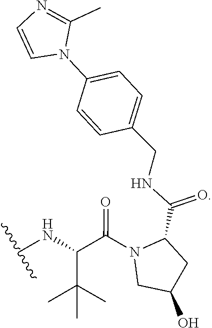 [SMILES: *N[C@H](C(=O)N1C[C@H](O)C[C@H]1C(=O)NCc1ccc(-n2ccnc2C)cc1)C(C)(C)C]

In some embodiments, LBM is

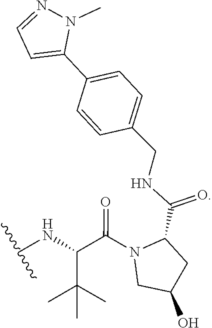 [SMILES: *N[C@H](C(=O)N1C[C@H](O)C[C@H]1C(=O)NCc1ccc(-c2ccnn2C)cc1)C(C)(C)C]

In some embodiments, LBM is

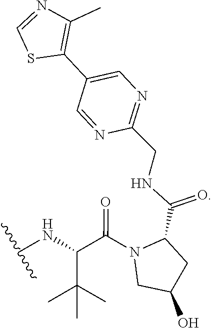 [SMILES: *N[C@H](C(=O)N1C[C@H](O)C[C@H]1C(=O)NCc1ncc(-c2scnc2C)cn1)C(C)(C)C]

In some embodiments, LBM is

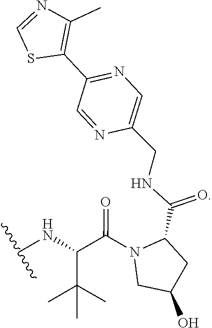 [SMILES: *N[C@H](C(=O)N1C[C@H](O)C[C@H]1C(=O)NCc1cnc(-c2scnc2C)cn1)C(C)(C)C]

In some embodiments, LBM is

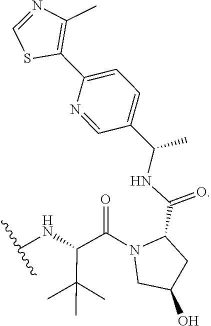 [SMILES: *N[C@H](C(=O)N1C[C@H](O)C[C@H]1C(=O)N[C@@H](C)c1ccc(-c2scnc2C)nc1)C(C)(C)C]

In some embodiments, LBM is

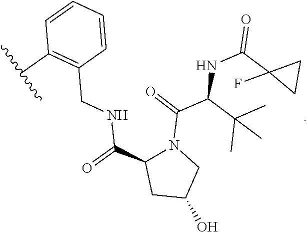 [SMILES: *c1ccccc1CNC(=O)[C@@H]1C[C@@H](O)CN1C(=O)[C@@H](NC(=O)C1(F)CC1)C(C)(C)C]

In some embodiments, LBM is

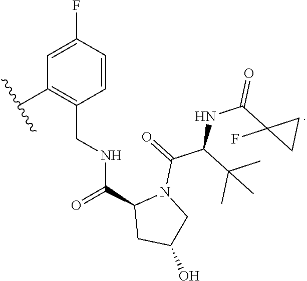 [SMILES: *c1cc(F)ccc1CNC(=O)[C@@H]1C[C@@H](O)CN1C(=O)[C@@H](NC(=O)C1(F)CC1)C(C)(C)C]

In some embodiments, LBM is

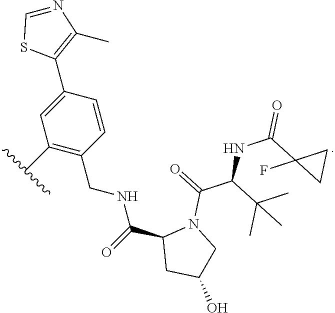 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

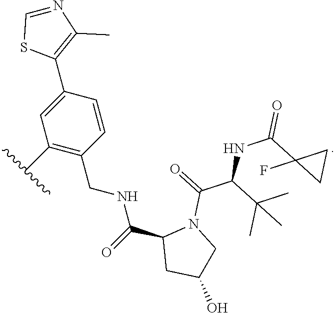 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

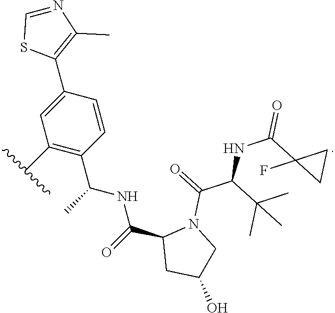 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C]

In some embodiments, LBM is

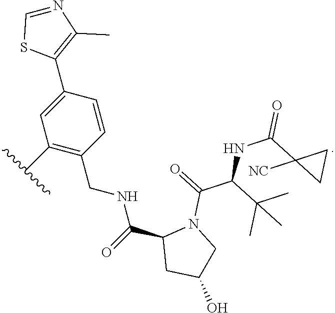 [SMILES: *c1cc(-c2scnc2C)ccc1CNC(=O)[C@@H]1C[C@@H](O)CN1C(=O)[C@@H](NC(=O)C1(C#N)CC1)C(C)(C)C]

In some embodiments, LBM is

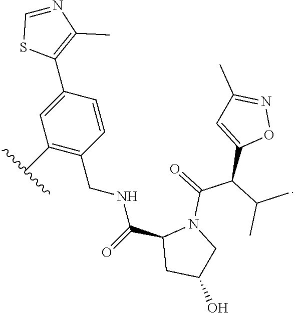 [SMILES: *c1cc(-c2scnc2C)ccc1CNC(=O)[C@@H]1C[C@@H](O)CN1C(=O)[C@@H](c1cc(C)no1)C(C)C]

In some embodiments, LBM is

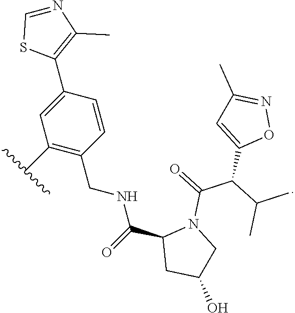 [SMILES: *c1cc(-c2scnc2C)ccc1CNC(=O)[C@@H]1C[C@@H](O)CN1C(=O)[C@H](c1cc(C)no1)C(C)C]

In some embodiments, LBM is

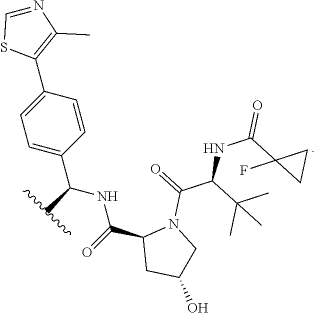 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

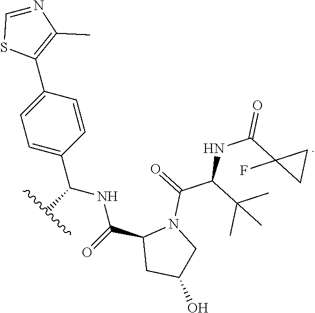 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

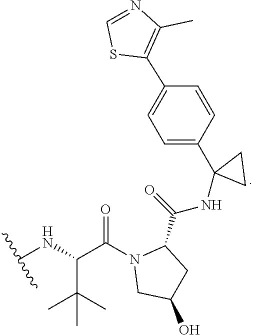 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

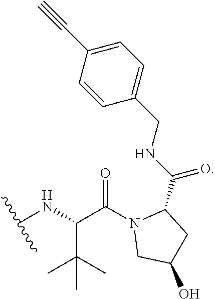 [SMILES: *N[C@H](C(=O)N1C[C@H](O)C[C@H]1C(=O)NCc1ccc(C#C)cc1)C(C)(C)C]

In some embodiments, LBM is

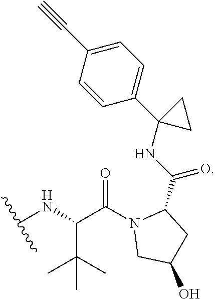 [SMILES: *N[C@H](C(=O)N1C[C@H](O)C[C@H]1C(=O)NC1(c2ccc(C#C)cc2)CC1)C(C)(C)C]

In some embodiments, LBM is

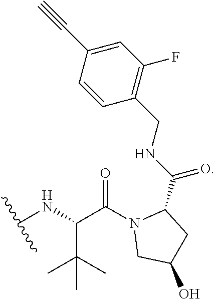 [SMILES: *N[C@H](C(=O)N1C[C@H](O)C[C@H]1C(=O)NCc1ccc(C#C)cc1F)C(C)(C)C]

In some embodiments, LBM is

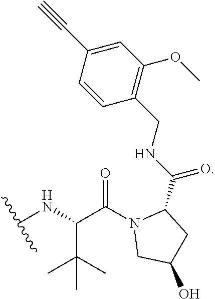 [SMILES: *N[C@H](C(=O)N1C[C@H](O)C[C@H]1C(=O)NCc1ccc(C#C)cc1OC)C(C)(C)C]

In some embodiments, LBM is

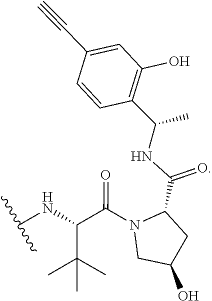 [SMILES: *N[C@H](C(=O)N1C[C@H](O)C[C@H]1C(=O)N[C@@H](C)c1ccc(C#C)cc1O)C(C)(C)C]

In some embodiments, LBM is

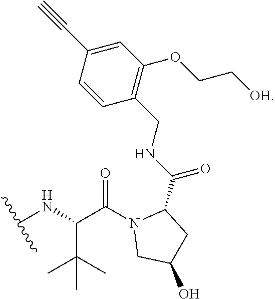 [SMILES: *N[C@H](C(=O)N1C[C@H](O)C[C@H]1C(=O)NCc1ccc(C#C)cc1OCCO)C(C)(C)C]

In some embodiments, LBM is

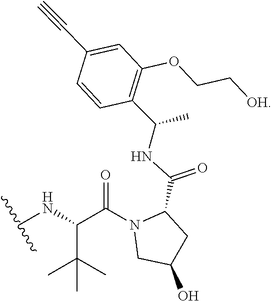 [SMILES: *N[C@H](C(=O)N1C[C@H](O)C[C@H]1C(=O)N[C@@H](C)c1ccc(C#C)cc1OCCO)C(C)(C)C]

In some embodiments, LBM is

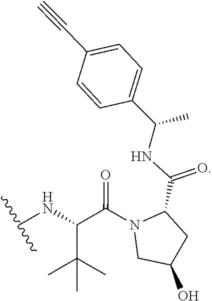 [SMILES: *N[C@H](C(=O)N1C[C@H](O)C[C@H]1C(=O)N[C@@H](C)c1ccc(C#C)cc1)C(C)(C)C]

In some embodiments, LBM is

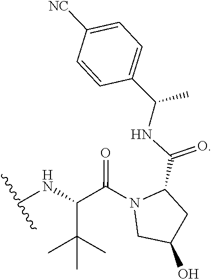 [SMILES: *N[C@H](C(=O)N1C[C@H](O)C[C@H]1C(=O)N[C@@H](C)c1ccc(C#N)cc1)C(C)(C)C]

In some embodiments, LBM is

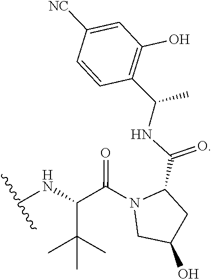 [SMILES: *N[C@H](C(=O)N1C[C@H](O)C[C@H]1C(=O)N[C@@H](C)c1ccc(C#N)cc1O)C(C)(C)C]

In some embodiments, LBM is

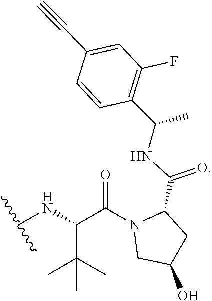 [SMILES: *N[C@H](C(=O)N1C[C@H](O)C[C@H]1C(=O)N[C@@H](C)c1ccc(C#C)cc1F)C(C)(C)C]

In some embodiments, LBM is

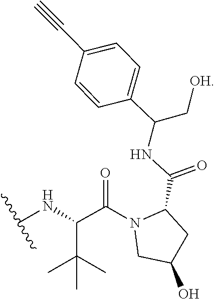 [SMILES: *N[C@H](C(=O)N1C[C@H](O)C[C@H]1C(=O)NC(CO)c1ccc(C#C)cc1)C(C)(C)C]

In some embodiments, LBM is

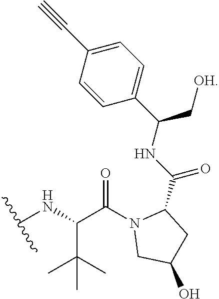 [SMILES: *N[C@H](C(=O)N1C[C@H](O)C[C@H]1C(=O)N[C@H](CO)c1ccc(C#C)cc1)C(C)(C)C]

In some embodiments, LBM is

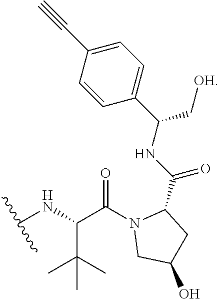 [SMILES: *N[C@H](C(=O)N1C[C@H](O)C[C@H]1C(=O)N[C@@H](CO)c1ccc(C#C)cc1)C(C)(C)C]

In some embodiments, LBM is

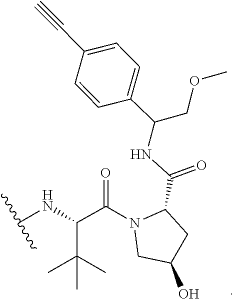 [SMILES: *N[C@H](C(=O)N1C[C@H](O)C[C@H]1C(=O)NC(COC)c1ccc(C#C)cc1)C(C)(C)C]

In some embodiments, LBM is

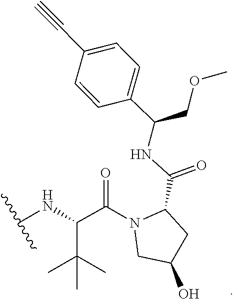 [SMILES: *N[C@H](C(=O)N1C[C@H](O)C[C@H]1C(=O)N[C@H](COC)c1ccc(C#C)cc1)C(C)(C)C]

In some embodiments, LBM is

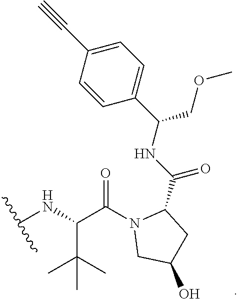 [SMILES: *N[C@H](C(=O)N1C[C@H](O)C[C@H]1C(=O)N[C@@H](COC)c1ccc(C#C)cc1)C(C)(C)C]

In some embodiments, LBM is

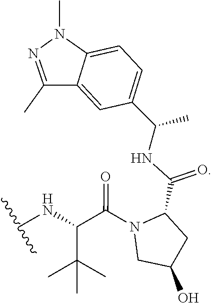 [SMILES: *N[C@H](C(=O)N1C[C@H](O)C[C@H]1C(=O)N[C@@H](C)c1ccc2c(c1)c(C)nn2C)C(C)(C)C]

In some embodiments, LBM is

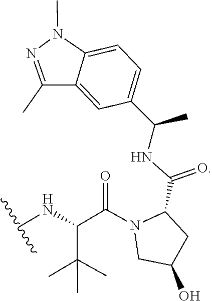 [SMILES: *N[C@H](C(=O)N1C[C@H](O)C[C@H]1C(=O)N[C@H](C)c1ccc2c(c1)c(C)nn2C)C(C)(C)C]

In some embodiments, LBM is

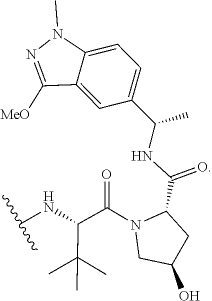 [SMILES: *N[C@H](C(=O)N1C[C@H](O)C[C@H]1C(=O)N[C@@H](C)c1ccc2c(c1)c(OC)nn2C)C(C)(C)C]

In some embodiments, LBM is

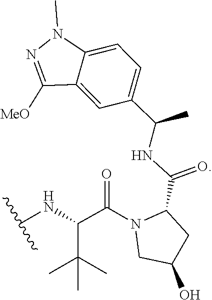 [SMILES: *N[C@H](C(=O)N1C[C@H](O)C[C@H]1C(=O)N[C@H](C)c1ccc2c(c1)c(OC)nn2C)C(C)(C)C]

In some embodiments, LBM is

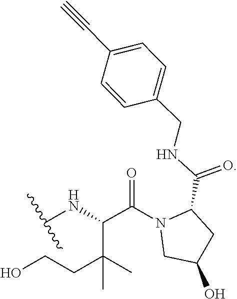 [SMILES: *N[C@H](C(=O)N1C[C@H](O)C[C@H]1C(=O)NCc1ccc(C#C)cc1)C(C)(C)CCO]

In some embodiments, LBM is

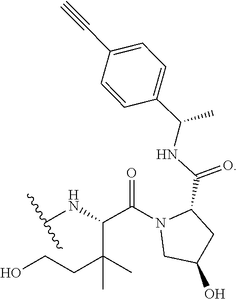 [SMILES: *N[C@H](C(=O)N1C[C@H](O)C[C@H]1C(=O)N[C@@H](C)c1ccc(C#C)cc1)C(C)(C)CCO]

In some embodiments, LBM is

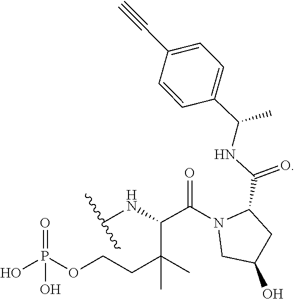 [SMILES: *N[C@H](C(=O)N1C[C@H](O)C[C@H]1C(=O)N[C@@H](C)c1ccc(C#C)cc1)C(C)(C)CCOP(=O)(O)O]

In some embodiments, LBM is

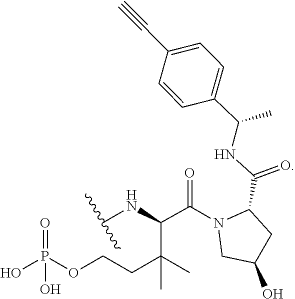 [SMILES: *N[C@@H](C(=O)N1C[C@H](O)C[C@H]1C(=O)N[C@@H](C)c1ccc(C#C)cc1)C(C)(C)CCOP(=O)(O)O]

In some embodiments, LBM is

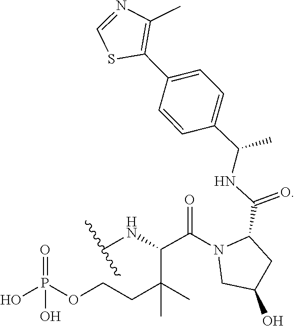 [SMILES: *N[C@H](C(=O)N1C[C@H](O)C[C@H]1C(=O)N[C@@H](C)c1ccc(-c2scnc2C)cc1)C(C)(C)CCOP(=O)(O)O]

In some embodiments, LBM is

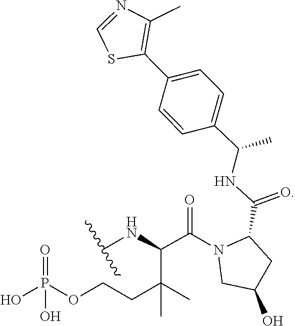 [SMILES: *N[C@@H](C(=O)N1C[C@H](O)C[C@H]1C(=O)N[C@@H](C)c1ccc(-c2scnc2C)cc1)C(C)(C)CCOP(=O)(O)O]

In some embodiments, LBM is

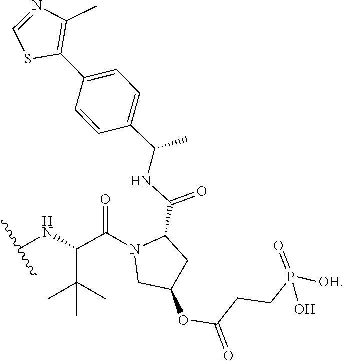 [SMILES: *N[C@H](C(=O)N1C[C@H](OC(=O)CCP(=O)(O)O)C[C@H]1C(=O)N[C@@H](C)c1ccc(-c2scnc2C)cc1)C(C)(C)C]

In some embodiments, LBM is

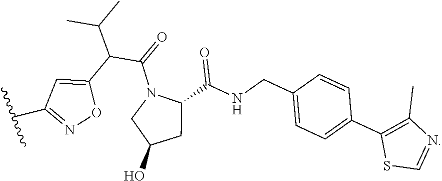 [SMILES: *c1cc(C(C(=O)N2C[C@H](O)C[C@H]2C(=O)NCc2ccc(-c3scnc3C)cc2)C(C)C)on1]

In some embodiments, LBM is

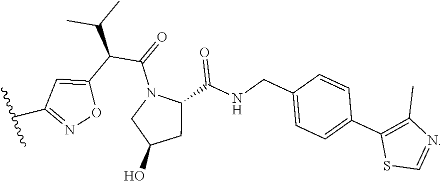 [SMILES: *c1cc([C@H](C(=O)N2C[C@H](O)C[C@H]2C(=O)NCc2ccc(-c3scnc3C)cc2)C(C)C)on1]

In some embodiments, LBM is

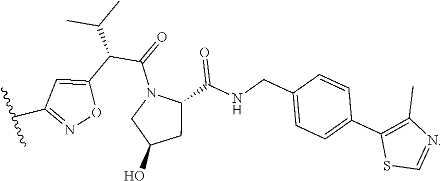 [SMILES: *c1cc([C@@H](C(=O)N2C[C@H](O)C[C@H]2C(=O)NCc2ccc(-c3scnc3C)cc2)C(C)C)on1]

In some embodiments, LBM is

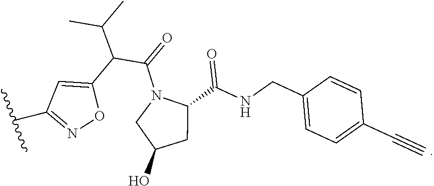 [SMILES: *c1cc(C(C(=O)N2C[C@H](O)C[C@H]2C(=O)NCc2ccc(C#C)cc2)C(C)C)on1]

In some embodiments, LBM is

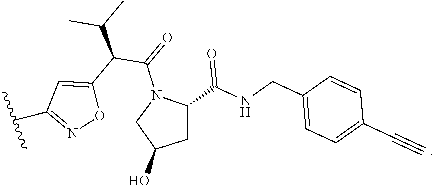 [SMILES: *c1cc([C@H](C(=O)N2C[C@H](O)C[C@H]2C(=O)NCc2ccc(C#C)cc2)C(C)C)on1]

In some embodiments, LBM is

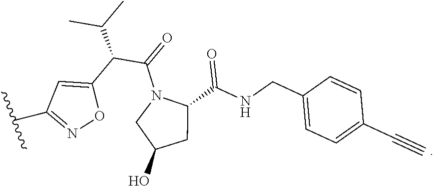 [SMILES: *c1cc([C@@H](C(=O)N2C[C@H](O)C[C@H]2C(=O)NCc2ccc(C#C)cc2)C(C)C)on1]

In some embodiments, LBM is

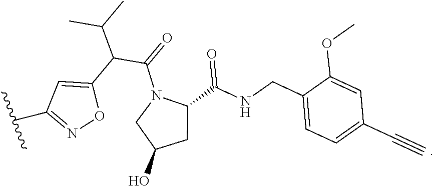 [SMILES: *c1cc(C(C(=O)N2C[C@H](O)C[C@H]2C(=O)NCc2ccc(C#C)cc2OC)C(C)C)on1]

In some embodiments, LBM is

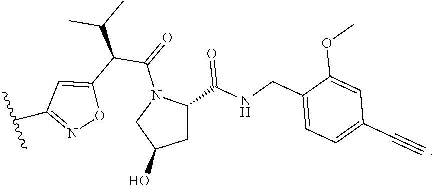 [SMILES: *c1cc([C@H](C(=O)N2C[C@H](O)C[C@H]2C(=O)NCc2ccc(C#C)cc2OC)C(C)C)on1]

In some embodiments, LBM is

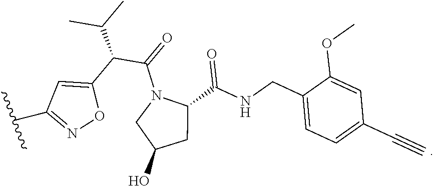 [SMILES: *c1cc([C@@H](C(=O)N2C[C@H](O)C[C@H]2C(=O)NCc2ccc(C#C)cc2OC)C(C)C)on1]

In some embodiments, LBM is

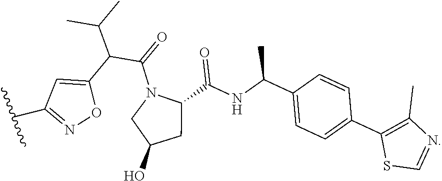 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

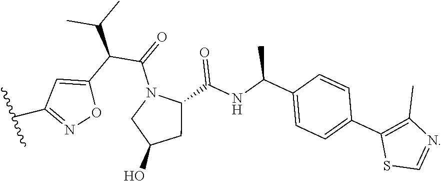 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

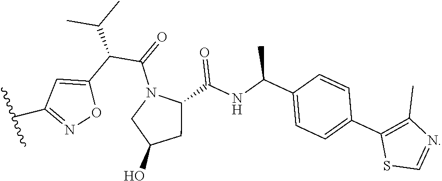 [SMILES: *c1cc([C@@H](C(=O)N2C[C@H](O)C[C@H]2C(=O)N[C@@H](C)c2ccc(-c3scnc3C)cc2)C(C)C)on1]

In some embodiments, LBM is

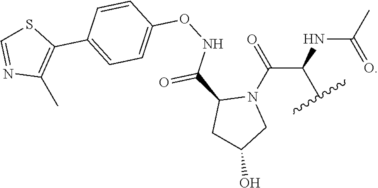 [SMILES: *[C@H](NC(C)=O)C(=O)N1C[C@H](O)C[C@H]1C(=O)NOc1ccc(-c2scnc2C)cc1]

In some embodiments, LBM is

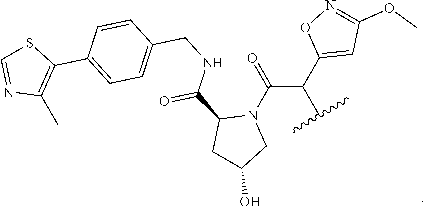 [SMILES: *C(C(=O)N1C[C@H](O)C[C@H]1C(=O)NCc1ccc(-c2scnc2C)cc1)c1cc(OC)no1]

In some embodiments, LBM is

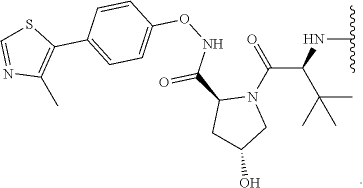 [SMILES: *N[C@H](C(=O)N1C[C@H](O)C[C@H]1C(=O)NOc1ccc(-c2scnc2C)cc1)C(C)(C)C]

In some embodiments, LBM is

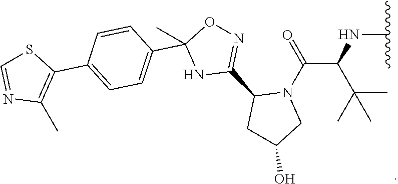 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

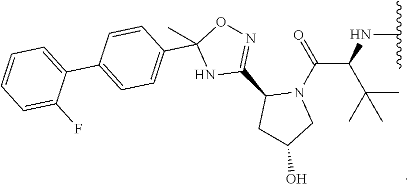 [SMILES: *N[C@H](C(=O)N1C[C@H](O)C[C@H]1C1=NOC(C)(c2ccc(-c3ccccc3F)cc2)N1)C(C)(C)C]

In some embodiments, LBM is

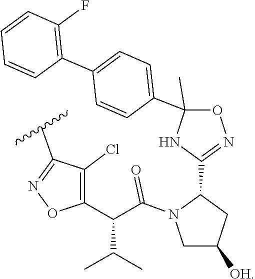 [SMILES: *c1noc([C@H](C(=O)N2C[C@H](O)C[C@H]2C2=NOC(C)(c3ccc(-c4ccccc4F)cc3)N2)C(C)C)c1Cl]

### In

some embodiments, LBM is

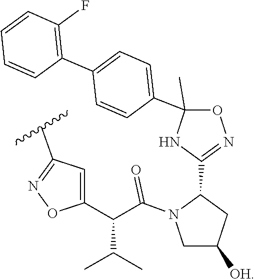 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

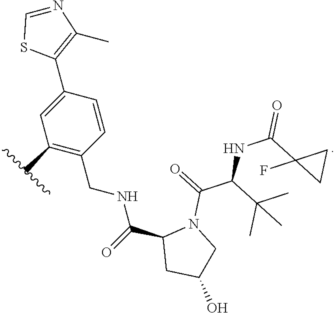

In some embodiments, LBM is

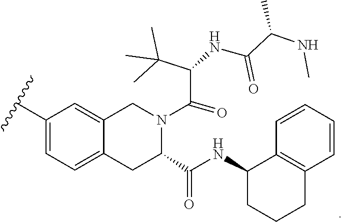 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

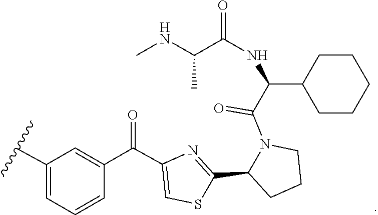 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

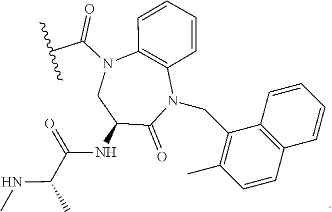 [SMILES: *C(=O)N1C[C@H](NC(=O)[C@H](C)NC)C(=O)N(Cc2c(C)ccc3ccccc23)c2ccccc21]

In some embodiments, LBM is

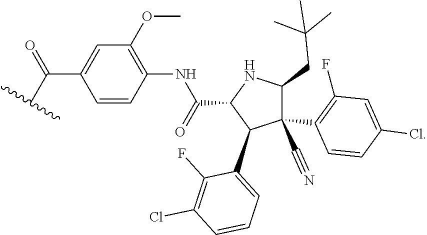 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

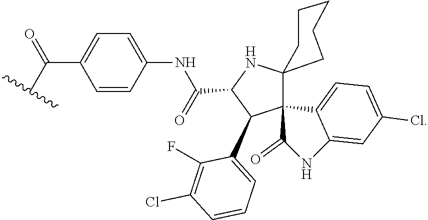 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

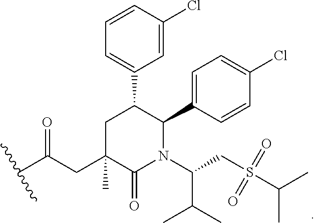 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

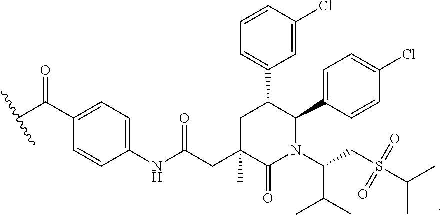 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

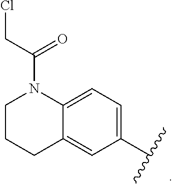 [SMILES: *c1ccc2c(c1)CCCN2C(=O)CCl]

In some embodiments, LBM is

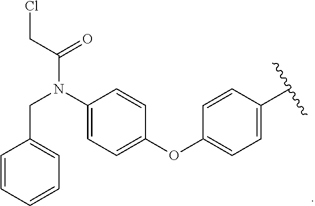 [SMILES: *c1ccc(Oc2ccc(N(Cc3ccccc3)C(=O)CCl)cc2)cc1]

In some embodiments, LBM is

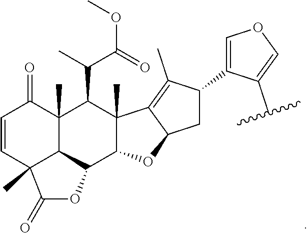 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

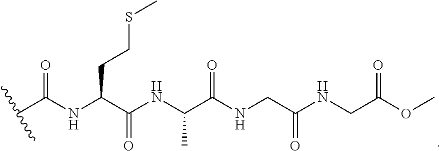 [SMILES: *C(=O)N[C@@H](CCSC)C(=O)N[C@@H](C)C(=O)NCC(=O)NCC(=O)OC]

In some embodiments, LBM is

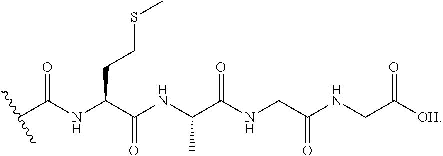 [SMILES: *C(=O)N[C@@H](CCSC)C(=O)N[C@@H](C)C(=O)NCC(=O)NCC(=O)O]

In some embodiments, LBM is

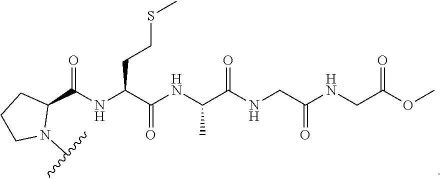 [SMILES: *N1CCC[C@H]1C(=O)N[C@@H](CCSC)C(=O)N[C@@H](C)C(=O)NCC(=O)NCC(=O)OC]

In some embodiments, LBM is

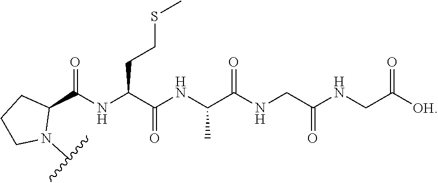 [SMILES: *N1CCC[C@H]1C(=O)N[C@@H](CCSC)C(=O)N[C@@H](C)C(=O)NCC(=O)NCC(=O)O]

In some embodiments, LBM is

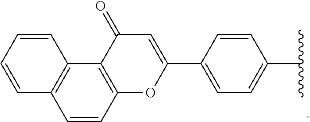 [SMILES: *c1ccc(-c2cc(=O)c3c(ccc4ccccc43)o2)cc1]

In some embodiments, LBM is

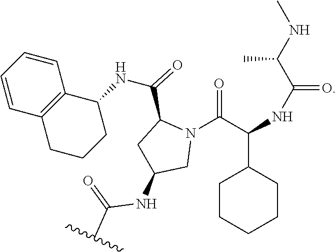 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

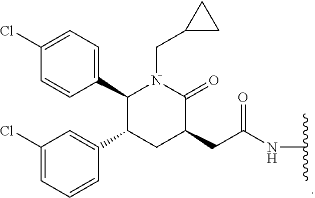 [SMILES: *NC(=O)C[C@H]1C[C@H](c2cccc(Cl)c2)[C@@H](c2ccc(Cl)cc2)N(CC2CC2)C1=O]

In some embodiments, LBM is

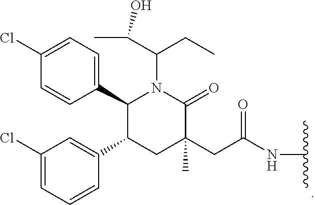 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

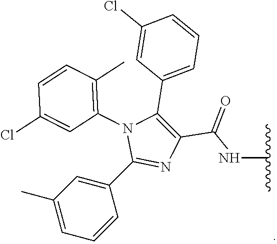 [SMILES: *NC(=O)c1nc(-c2cccc(C)c2)n(-c2cc(Cl)ccc2C)c1-c1cccc(Cl)c1]

In some embodiments, LBM is

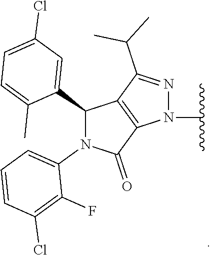 [SMILES: *n1nc(C(C)C)c2c1C(=O)N(c1cccc(Cl)c1F)[C@H]2c1cc(Cl)ccc1C]

In certain embodiments, the present invention provides a compound of formula I, wherein LBM is a CRBN E3 ubiquitin ligase binding moiety thereby forming a compound of formula I-II:

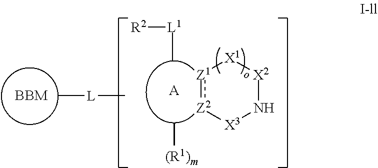

or a pharmaceutically acceptable salt thereof, wherein L and BBM are as defined above and described in embodiments herein, wherein:
each X¹ is independently —CH₂—, —O—, —NR—, —CF₂—,

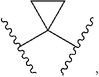 [SMILES: *C1(*)CC1]

### —C(O)—, —C(S)—, or

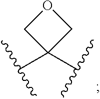 [SMILES: *C1(*)COC1]

X² and X³ are independently —CH₂—, —C(O)—, —C(S)—, or

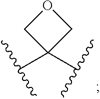 [SMILES: *C1(*)COC1]

Z¹ and Z² are independently a carbon atom or a nitrogen atom:
   Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂ (OR), —CR₂ (NR₂), —C(O)R, —C(O)OR, —C(O)NR₂, —C(O)N(R) OR, —OC(O)R, —OC(O)NR₂, —C(S) NR₂, —N(R)C(O)OR, —N(R)C(O)R, —N(R)C(O)NR₂, —N(R)S(O)₂R, —OP(O)R₂, —OP(O)(OR)₂, OP(O)(OR)NR₂, —OP(O)(NR₂)₂, —Si(OR) R₂, and —SiR₃; or two R₁ groups are optionally taken together to form an optionally substituted 5-8 membered partially unsaturated or aryl fused ring having 0-2 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R₂ is selected from

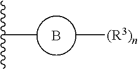

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R₃ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂ (OR), —CR₂ (NR₂), —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
is 0, 1, or 2.

As defined above and described herein X¹ is a covalent bond, —CH₂—, —O—, —NR—, —CF₂—,

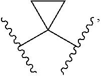 [SMILES: *C1(*)CC1]

### —C(O)—, —C(S)—, or

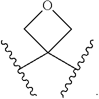 [SMILES: *C1(*)COC1]

In some embodiments, X¹ is a covalent bond. In some embodiments, X¹ is —CH₂—. In some embodiments, X¹ is

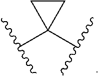 [SMILES: *C1(*)CC1]

In some embodiments. X¹ is —NR—. In some embodiments, X¹ is —CF₂—. In some some embodiments, X¹ is

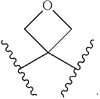 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

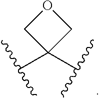 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

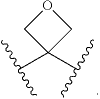 [SMILES: *C1(*)COC1]

In certain embodiments, X² and X³ are independently selected from those shown in the compounds of Table 1.

As defined above and described herein, X⁴ is a covalent bond, —CH₂—, —CR₂—, —O—, —NR—, —CF₂—,

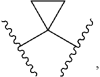 [SMILES: *C1(*)CC1]

### —C(O)—, —C(S)—, or

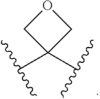 [SMILES: *C1(*)COC1]

In some embodiments, X⁴ is a covalent bond. In some embodiments, X⁴ is —CH₂—. In some embodiments, X⁴ is —CR₂—. In some embodiments, X⁴ is —O—. In some embodiments, X⁴ is —NR—. In some embodiments, X⁴ is —CF₂—. In some embodiments, X⁴ is

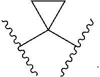 [SMILES: *C1(*)CC1]

In some embodiments, X⁴ is —C(O)—. In some embodiments, X⁴ is —C(S)—. In some embodiments, X⁴ is

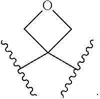 [SMILES: *C1(*)COC1]

In certain embodiments, X⁴ is selected from those shown in the compounds of Table 1.

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In some embodiments, Ring A is

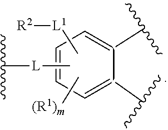

In certain embodiments, Ring A is selected from those shown in the compounds of Table 1.

In some embodiments, Ring C is a spiro-fused ring selected from a 4-10 membered saturated or partially unsaturated mono- or bicyclic carbocyclic or heterocyclic ring having 1-3 heteroatoms independently selected from nitrogen, oxygen, and sulfur. In some embodiments, Ring C is optionally further substituted with 1-2 oxo groups.

In certain embodiments, Ring C is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂ (OR), —CR₂ (NR₂), —C(O)R, —C(O)OR, —C(O)NR₂, —C(O)N(R) OR, —OC(O)R, —OC(O)NR₂, —C(S) NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR) R₂, and —SiR₃, or two R₁ groups are optionally taken together to form an optionally substituted 5-8 membered partially unsaturated or aryl fused ring having 0-2 heteroatoms independently selected from nitrogen, oxygen, and sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂ (OR). In some embodiments, R¹ is —CR₂ (NR₂). In some embodiments, R¹ is —C(O)R. In some embodiments, R¹ is —C(O)OR. In some embodiments, R¹ is —C(O)NR₂. In some embodiments, R¹ is —C(O)N(R) OR. In some embodiments, R¹ is —OC(O)R. In some embodiments, R¹ is —OC(O)NR₂. In some embodiments, R¹ is —C(S) NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR) R₂. In some embodiments, R¹ is —SiR₃. In some embodiments, two R₁ groups are optionally taken together to form an optionally substituted 5-8 membered partially unsaturated or aryl fused ring having 0-2 heteroatoms independently selected from nitrogen, oxygen, and sulfur.

In some embodiments, R¹ is fluoro. In some embodiments, R¹ is

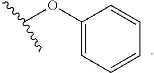 [SMILES: *Oc1ccccc1]

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁-6 aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R₂ is selected from

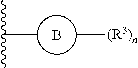

or hydrogen.

In some embodiment R₂ is

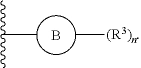

In some embodiments, R₂ is hydrogen.

In certain embodiments, R₂ is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring Bis selected from those shown in the compounds of Table 1.

As defined above and described herein, each R₃ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂ (OR), —CR₂ (NR₂), —C(O)R, —C(O)OR, —C(O)NR₂, —C(O)N(R) OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R₃ is hydrogen. In some embodiments, R₃ is deuterium. In some embodiments, R₃ is R⁴. In some embodiments, R₃ is halogen. In some embodiments, R₃ is —CN. In some embodiments, R₃ is —NO₂. In some embodiments, R₃ is —OR. In some embodiments, R₃ is —SR. In some embodiments, R₃ is —NR₂. In some embodiments, R₃ is —S(O)₂R. In some embodiments, R₃ is —S(O)₂NR₂. In some embodiments, R₃ is —S(O)R. In some embodiments, R₃ is —CF₂R. In some embodiments, R₃ is —CF₃. In some embodiments, R₃ is —CR₂ (OR). In some embodiments, R₃ is —CR₂ (NR₂). In some embodiments, R₃ is —C(O)R. In some embodiments, R₃ is —C(O)OR. In some embodiments, R₃ is —C(O)NR₂. In some embodiments, R₃ is —C(O)N(R) OR. In some embodiments, R₃ is —OC(O)R. In some embodiments, R₃ is —OC(O)NR₂. In some embodiments, R₃ is —N(R)C(O)OR. In some embodiments, R₃ is —N(R)C(O)R. In some embodiments, R₃ is —N(R)C(O)NR₂. In some embodiments, R₃ is —N(R)S(O)₂R. In some embodiments, R₃ is —OP(O)R₂. In some embodiments, R₃ is —OP(O)(OR)₂. In some embodiments, R₃ is —OP(O)(OR)NR₂. In some embodiments, R₃ is —OP(O)(NR₂)₂. In some embodiments, R₃ is —SiR₃.

In certain embodiments, R₃ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R₄ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1. As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1.

As defined above and described herein, o is 0, 1, or 2.

In some embodiments, n is 0. In some embodiments, n is 1. In some embodiments, m is 2.

In certain embodiments, o is selected from those shown in the compounds of Table 1.

In some embodiments, the present invention provides a compound of formula I-cc, wherein

Ring A is benzo, o is 1, X¹ is —CH₂—, X² and X³ are —C(O)—, and Z¹ and Z² are carbon atoms as shown, to provide a compound of formula I-cc-1:

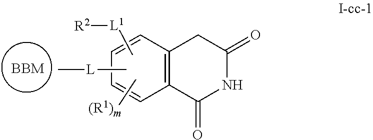

or a pharmaceutically acceptable salt thereof, wherein each of BBM, L, L¹, R¹, R², and m is as defined above and described in embodiments herein, both singly and in combination.

In some embodiments, the present invention provides a compound of formula I-cc, wherein Ring A is benzo, o is 1, X¹, X² and X³ are —C(O)—, and Z¹ and Z² are carbon atoms as shown, to provide a compound of formula I-cc-12:

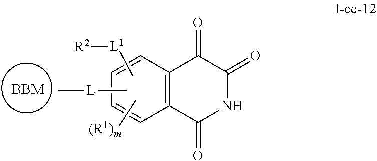

or a pharmaceutically acceptable salt thereof, wherein each of BBM, L, L¹, R¹, R², and m is as defined above and described in embodiments herein, both singly and in combination.

In some embodiments, LBM is

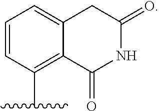 [SMILES: *c1cccc2c1C(=O)NC(=O)C2]

In some embodiments, LBM is

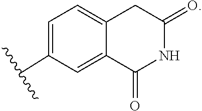 [SMILES: *c1ccc2c(c1)C(=O)NC(=O)C2]

In some embodiments, LBM is

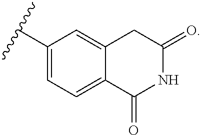 [SMILES: *c1ccc2c(c1)CC(=O)NC2=O]

In some embodiments, LBM is

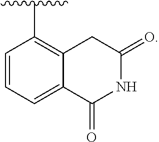 [SMILES: *c1cccc2c1CC(=O)NC2=O]

In some embodiments, LBM is

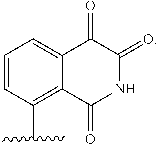 [SMILES: *c1cccc2c1C(=O)NC(=O)C2=O]

In some embodiments, LBM is

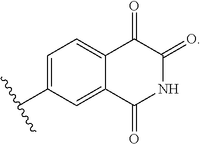 [SMILES: *c1ccc2c(c1)C(=O)NC(=O)C2=O]

In some embodiments, LBM is

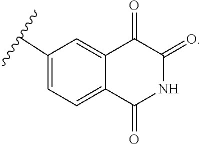 [SMILES: *c1ccc2c(c1)C(=O)C(=O)NC2=O]

In some embodiments, LBM is

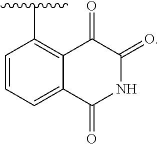 [SMILES: *c1cccc2c1C(=O)C(=O)NC2=O]

In some embodiments, LBM is selected from those in Table 1.

In certain embodiments, the present invention provides a compound of formula I, wherein LBM is a RPN13 binding moiety thereby forming a compound of formula I-o-1:

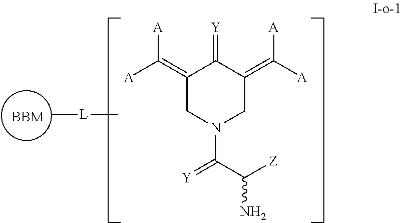

or a pharmaceutically acceptable salt thereof, wherein L and BBM are as defined above and described in embodiments herein, and wherein each of the variables A, Y, and Z is as described and defined in WO 2019/165229, the entirety of each of which is herein incorporated by reference.

In certain embodiments, the present invention provides a compound of formula I, wherein LBM is a Ubr1 binding moiety as described in Shanmugasundaram, K. et al, J. Bio. Chem. 2019, doi: 10.1074/jbc.AC119.010790, the entirety of each of which is herein incorporated by reference, thereby forming a compound of formula I-o-2 or I-o-3:

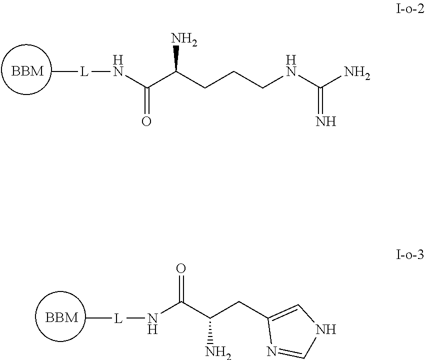

or a pharmaceutically acceptable salt thereof, wherein L and BBM are as defined above and described in embodiments herein.

In certain embodiments, the present invention provides a compound of formula I, wherein LBM is a cereblon binding moiety thereby forming a compound of formula I-o-4:

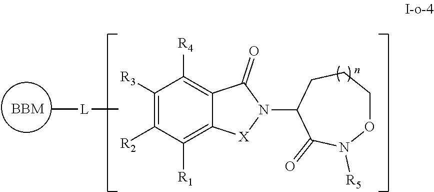

or a pharmaceutically acceptable salt thereof, wherein L and BBM are as defined above and described in embodiments herein, and wherein each of the variables R₁, R₂, R₃, R₄, R₅, Q, X, and n is as described and defined in US 2019/276474, the entirety of each of which is herein incorporated by reference.

In certain embodiments, the present invention provides a compound of formula I, wherein LBM is a cereblon E3 ubiquitin ligase binding moiety thereby forming a compound of formula I-o-5, I-o-6, 1-o-7 or I-o-8:

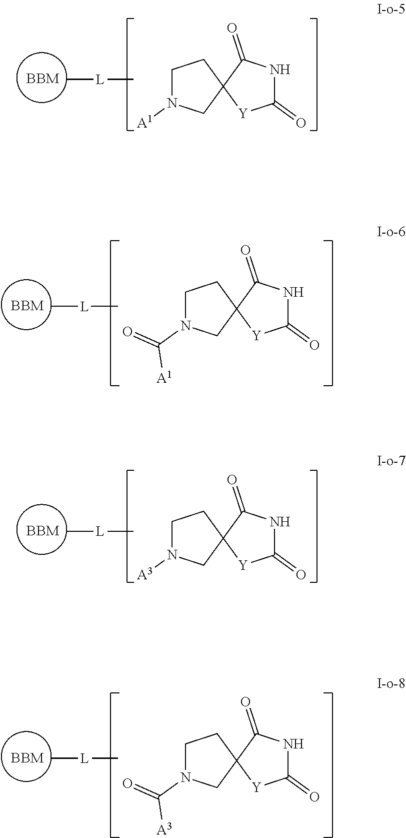

or a pharmaceutically acceptable salt thereof, wherein L and BBM are as defined above and described in embodiments herein, and wherein each of the variables Y, A′, and A3 is as described and defined in WO 2019/236483, the entirety of each of which is herein incorporated by reference.

In certain embodiments, the present invention provides a compound of formula I, wherein LBM is human kelch-like ECH-associated protein I (KEAP1) of formula I-o-9:

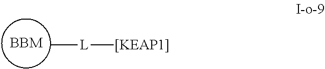

or a pharmaceutically acceptable salt thereof.

In certain embodiments, the present invention provides a compound of formula I, wherein LBM is KEAP1 binding moiety as recited in Lu et al., Euro. J. Med. Chem., 2018, 146:251-9, thereby forming a compound of formula I-o-10:

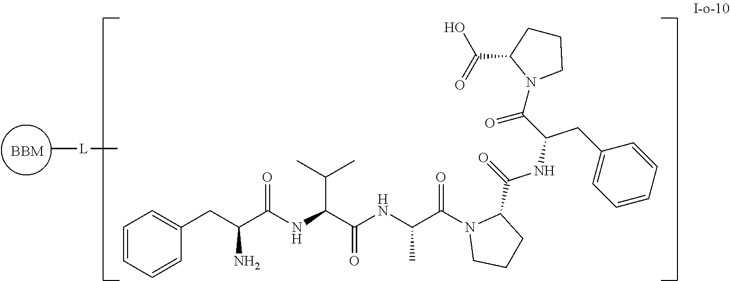

or a pharmaceutically acceptable salt thereof, wherein L and BBM are as defined above and described in embodiments herein.

In certain embodiments, the present invention provides a compound of formula I, wherein LBM is KEAP1-NRF2 binding moiety thereby forming a compound of formula I-o-11 or I-o-12:

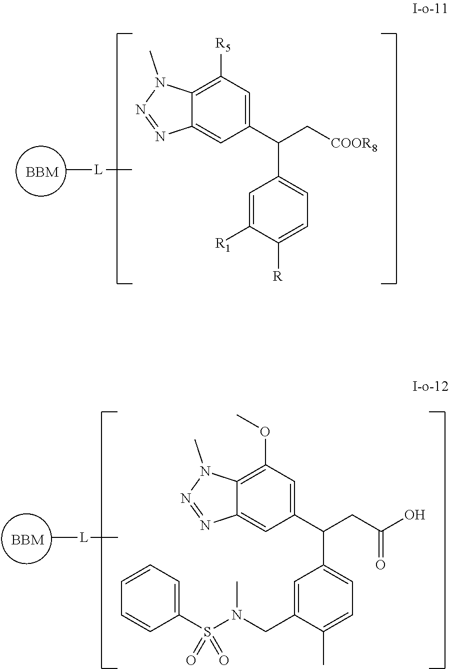

or a pharmaceutically acceptable salt thereof, wherein L and BBM are as defined above and described in embodiments herein, wherein each of the variables R, R₁, R₅, and R₈ is as described and defined in WO 2020/018788, the entirety of each of which is herein incorporated by reference.

In certain embodiments, the present invention provides a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-o-13 or I-o-14:

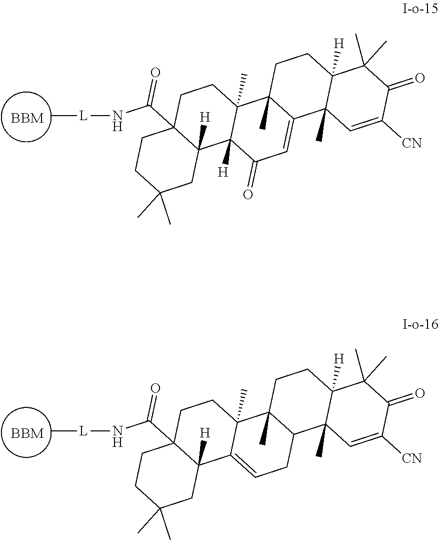

or a pharmaceutically acceptable salt thereof, wherein L and BBM are as defined above and described in embodiments herein.

### Lysine Mimetic

In some embodiments, DIM is LBM as described above and herein. In some embodiments, DIM is a lysine mimetic. In some embodiments, the covalent attachment of ubiquitin to BCL-XL protein is achieved through the action of a lysine mimetic. In some embodiments, upon the binding of a compound of formula I to BCL-XL, the DIM moiety that mimics a lysine undergoes ubiquitination thereby marking BCL-XL for degradation via the Ubiquitin-Proteasome Pathway (UPP).

In some embodiments, DIM is

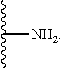 [SMILES: *N]

In some embodiments, DIM is

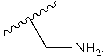 [SMILES: *CN]

In some embodiments, DIM is

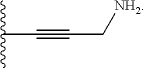 [SMILES: *C#CCN]

In some embodiments, DIM is selected from those depicted in Table 1, below.

In some embodiments, the present invention provides the compound of formula I as a compound of formula I-p-1:

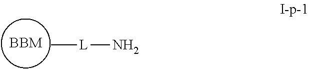

or a pharmaceutically acceptable salt thereof, wherein each of BBM and L is as defined above and described in embodiments herein, both singly and in combination.

In some embodiments, the present invention provides the compound of formula I as a compound of formula I-p-2:

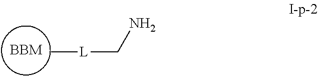

or a pharmaceutically acceptable salt thereof, wherein each of BBM and L is as defined above and described in embodiments herein, both singly and in combination.

In some embodiments, the present invention provides the compound of formula I as a compound of formula I-p-3:

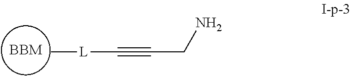

or a pharmaceutically acceptable salt thereof, wherein each of BBM and L is as defined above and described in embodiments herein, both singly and in combination.

In certain embodiments, the present invention provides a compound of formula I, wherein DIM is a lysine mimetic

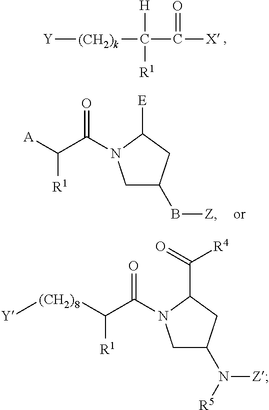

thereby forming a compound of Formulae I-q-1, I-q-2, or I-q-3, respectively:

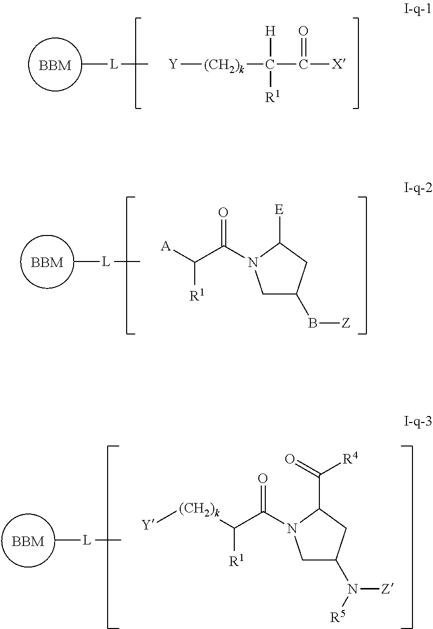

or a pharmaceutically acceptable salt thereof, wherein L and BBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

### Hydrogen Atom

In some embodiments, DIM is a hydrogen atom. In some embodiments, the covalent attachment of ubiquitin to BCL-XL protein is achieved through a provided compound wherein DIM is a hydrogen atom. In some embodiments, upon the binding of a compound of formula I to BCL-XL, the DIM moiety being hydrogen effectuates ubiquitination thereby marking BCL-XL for degradation via the Ubiquitin-Proteasome Pathway (UPP).

In some embodiments, DIM is selected from those depicted in Table 1, below.

In some embodiments, the present invention provides the compound of formula I wherein DIM is a hydrogen atom, thereby forming a compound of formula I-r:

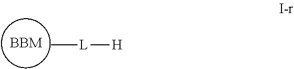

or a pharmaceutically acceptable salt thereof, wherein each of BBM and L is as defined above and described in embodiments herein, both singly and in combination.

### Linker (L)

As defined above and described herein, L is a bivalent moiety that connects to BBM to DIM.

In some embodiments, L is a bivalent moiety that connects BBM to DIM. In some embodiments, L is a bivalent moiety that connects BBM to LBM. In some embodiments, L is a bivalent moiety that connects BBM to a lysine mimetic.

In some embodiments, L is a covalent bond or a bivalent, saturated or partially unsaturated, straight or branched C₁₋₅₀ hydrocarbon chain, wherein 0-10 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

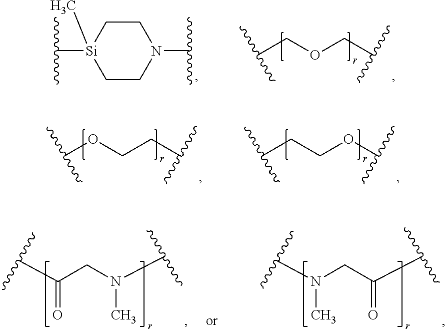

wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur, wherein r is 0, 1, 2, 3, 4, 5, 6, 7, 8, 9, or 10, and wherein R is as defined and described herein.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 4-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 4-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is

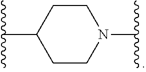 [SMILES: *C1CCN(*)CC1]

In some embodiments, -Cy- is

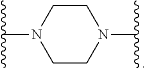 [SMILES: *N1CCN(*)CC1]

In some embodiments, -Cy- is

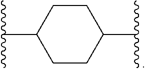 [SMILES: *C1CCC(*)CC1]

In some embodiments, -Cy- is

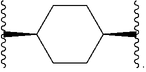 [SMILES: *[C@H]1CC[C@@H](*)CC1]

In some embodiments, -Cy- is

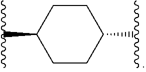 [SMILES: *[C@H]1CC[C@H](*)CC1]

In some embodiments, -Cy- is

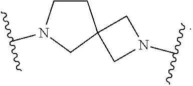 [SMILES: *N1CCC2(C1)CN(*)C2]

In some embodiments, -Cy- is

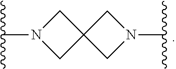 [SMILES: *N1CC2(C1)CN(*)C2]

In some embodiments, -Cy- is

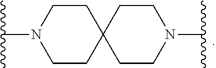 [SMILES: *N1CCC2(CC1)CCN(*)CC2]

In some embodiments, -Cy- is selected from those as depicted in the compounds of Table 1, below:

In some embodiments, L is —NR—(C₁₋₁₀ aliphatic)-. In some embodiments, L is —(C₁₋₁₀ aliphatic)-NR—(C₁₋₁₀ aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH₂CH₂O)₁₋₁₀ CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀ aliphatic)-. In some embodiments, L is —(C₁₋₁₀ aliphatic)-NRCO—(CH₂CH₂O) I-10CH₂CH₂—. In some embodiments, L is -Cy-NRCO—(C₁₋₁₀ aliphatic)-. In some embodiments, L is -Cy-(C₁-10 aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments. L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(C₂CH₂O)₁₋₁₀CH₂CH₂—. In some embodiments, Lis -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, Lis -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀—Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is —Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is —CONHSO₂-Cy-CO—. In some embodiments, L is —CONHSO₂—(CH₂)₁₋₁₀—CO—. In some embodiments, L is —CONHSO₂—(CH₂)₁₋₁₀-Cy-CO—. In some embodiments, L is —CONHSO₂-Cy-(CH₂)₁₋₁₀—CO—. In some embodiments, L is —CONHSO₂-Cy-Cy-CO—.

In some embodiments, L is —O—. In some embodiments, L is

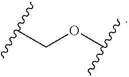 [SMILES: *CO*]

In some embodiments, L is

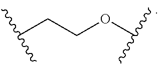 [SMILES: *CCO*]

In some embodiments, L is

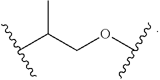 [SMILES: *OCC(*)C]

In some embodiments, L is

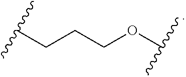 [SMILES: *CCCO*]

In some embodiments, L is

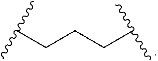 [SMILES: *CCC*]

In some embodiments, L is

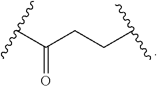 [SMILES: *CCC(*)=O]

In some embodiments, L is

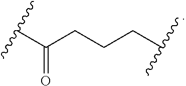 [SMILES: *CCCC(*)=O]

In some embodiments, L is

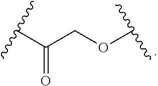 [SMILES: *OCC(*)=O]

In some embodiments, L is

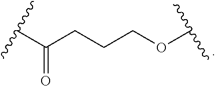 [SMILES: *OCCCC(*)=O]

In some embodiments, L is

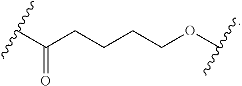 [SMILES: *OCCCCC(*)=O]

In some embodiments, L is

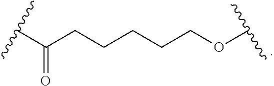 [SMILES: *OCCCCCC(*)=O]

In some embodiments, L is

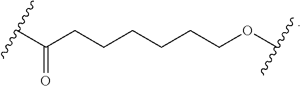 [SMILES: *OCCCCCCC(*)=O]

In some embodiments, L is

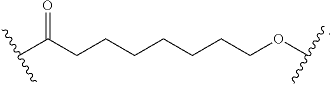 [SMILES: *OCCCCCCCC(*)=O]

In some embodiments, L is

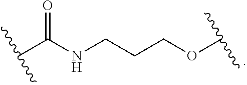 [SMILES: *OCCCNC(*)=O]

In some embodiments, L is

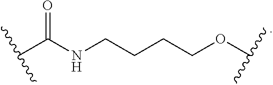 [SMILES: *OCCCCNC(*)=O]

In some embodiments, L is

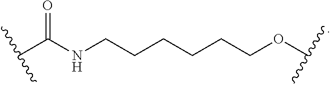 [SMILES: *OCCCCCCNC(*)=O]

In some embodiments, L is

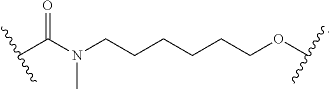 [SMILES: *OCCCCCCN(C)C(*)=O]

In some embodiments, L is

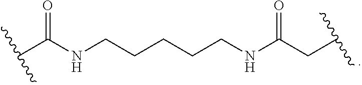 [SMILES: *CC(=O)NCCCCCNC(*)=O]

In some embodiments, L is

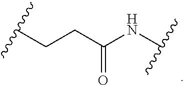 [SMILES: *CCC(=O)N*]

In some embodiments, L is

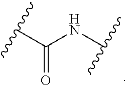 [SMILES: *NC(*)=O]

In some embodiments, L is

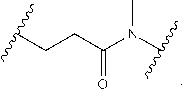 [SMILES: *CCC(=O)N(*)C]

In some embodiments, L is

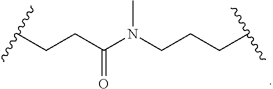 [SMILES: *CCCN(C)C(=O)CC*]

In some embodiments, L is

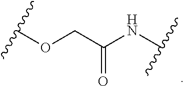 [SMILES: *NC(=O)CO*]

In some embodiments, L is

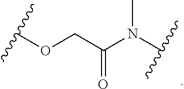 [SMILES: *OCC(=O)N(*)C]

In some embodiments, L is

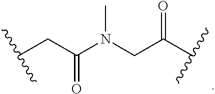 [SMILES: *CC(=O)N(C)CC(*)=O]

In some embodiments, L is

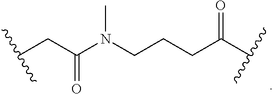 [SMILES: *CC(=O)N(C)CCCC(*)=O]

In some embodiments, L is

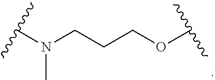 [SMILES: *OCCCN(*)C]

In some embodiments, L is

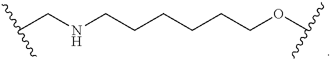 [SMILES: *CNCCCCCCO*]

In some embodiments, L is

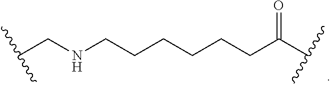 [SMILES: *CNCCCCCCC(*)=O]

In some embodiments, L is

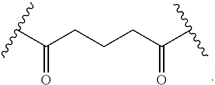 [SMILES: *C(=O)CCCC(*)=O]

In some embodiments, L is

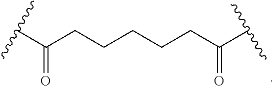 [SMILES: *C(=O)CCCCCC(*)=O]

In some embodiments, L is

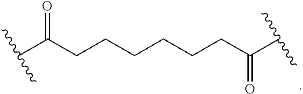 [SMILES: *C(=O)CCCCCCC(*)=O]

In some embodiments, L is

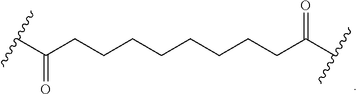 [SMILES: *C(=O)CCCCCCCCC(*)=O]

In some embodiments, L is

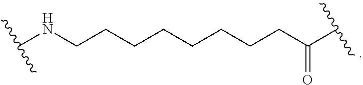 [SMILES: *NCCCCCCCCC(*)=O]

In some embodiments, L is

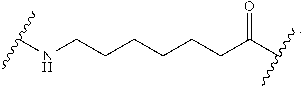 [SMILES: *NCCCCCCC(*)=O]

In some embodiments, L is

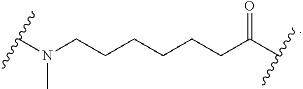 [SMILES: *C(=O)CCCCCCN(*)C]

In some embodiments, L is

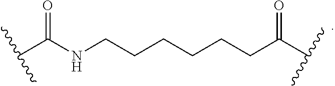 [SMILES: *C(=O)CCCCCCNC(*)=O]

In some embodiments, L is

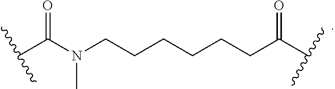 [SMILES: *C(=O)CCCCCCN(C)C(*)=O]

In some embodiments, L is

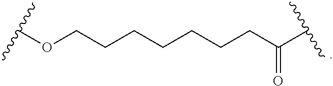 [SMILES: *OCCCCCCCC(*)=O]

In some embodiments, L is

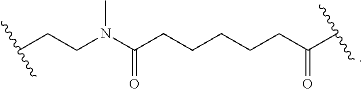 [SMILES: *CCN(C)C(=O)CCCCCC(*)=O]

In some embodiments, L is

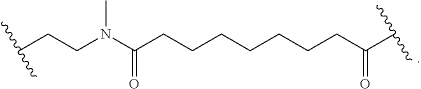 [SMILES: *CCN(C)C(=O)CCCCCCCC(*)=O]

In some embodiments, L is

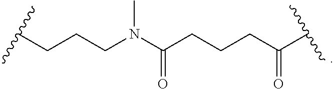 [SMILES: *CCCN(C)C(=O)CCCC(*)=O]

In some embodiments, L is

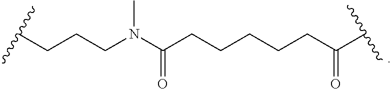 [SMILES: *CCCN(C)C(=O)CCCCCC(*)=O]

In some embodiments, L is

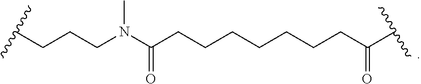 [SMILES: *CCCN(C)C(=O)CCCCCCCC(*)=O]

In some embodiments, L is

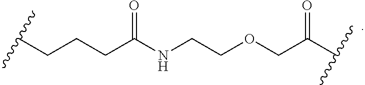 [SMILES: *CCCC(=O)NCCOCC(*)=O]

In some embodiments, L is

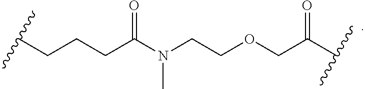 [SMILES: *CCCC(=O)N(C)CCOCC(*)=O]

In some embodiments, L is

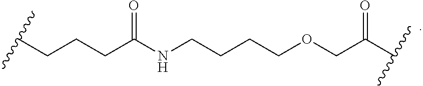 [SMILES: *CCCC(=O)NCCCCOCC(*)=O]

In some embodiments, L is

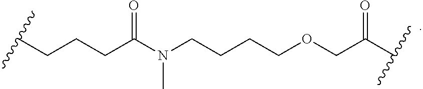 [SMILES: *CCCC(=O)N(C)CCCCOCC(*)=O]

In some embodiments, L is

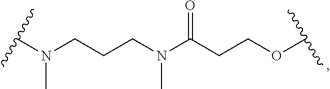 [SMILES: *OCCC(=O)N(C)CCCN(*)C]

In some embodiments, L is

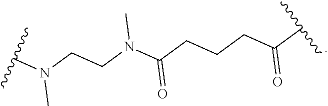 [SMILES: *C(=O)CCCC(=O)N(C)CCN(*)C]

In some embodiments, L is

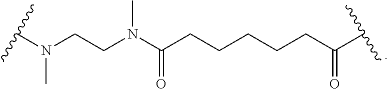 [SMILES: *C(=O)CCCCCC(=O)N(C)CCN(*)C]

In some embodiments, L is

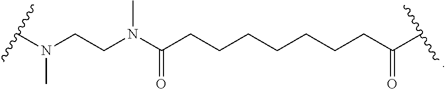 [SMILES: *C(=O)CCCCCCCC(=O)N(C)CCN(*)C]

In some embodiments, L is

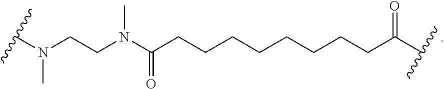 [SMILES: *C(=O)CCCCCCCCC(=O)N(C)CCN(*)C]

In some embodiments, L is

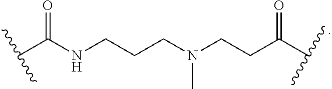 [SMILES: *C(=O)CCN(C)CCCNC(*)=O]

In some embodiments, L is

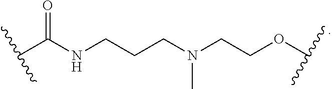 [SMILES: *OCCN(C)CCCNC(*)=O]

In some embodiments, L is

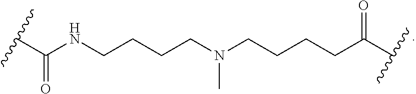 [SMILES: *C(=O)CCCCN(C)CCCCNC(*)=O]

In some embodiments, L is

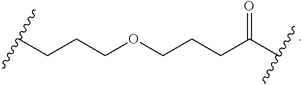 [SMILES: *CCCOCCCC(*)=O]

In some embodiments, L is

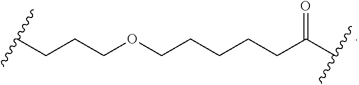 [SMILES: *CCCOCCCCCC(*)=O]

In some embodiments, L is

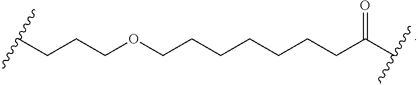 [SMILES: *CCCOCCCCCCCC(*)=O]

In some embodiments, L is

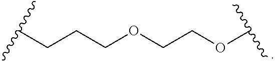 [SMILES: *CCCOCCO*]

In some embodiments, L is

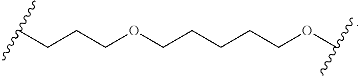 [SMILES: *CCCOCCCCCO*]

In some embodiments, L is

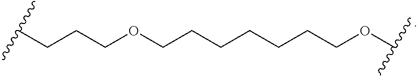 [SMILES: *CCCOCCCCCCCO*]

In some embodiments, L is

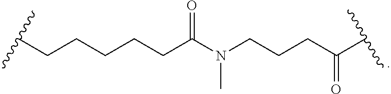 [SMILES: *CCCCCC(=O)N(C)CCCC(*)=O]

In some embodiments, L is

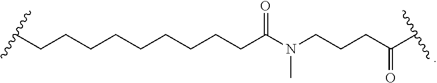 [SMILES: *CCCCCCCCCC(=O)N(C)CCCC(*)=O]

In some embodiments, L is

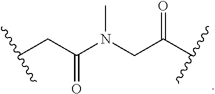 [SMILES: *CC(=O)N(C)CC(*)=O]

In some embodiments, L is

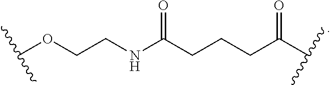 [SMILES: *OCCNC(=O)CCCC(*)=O]

In some embodiments, L is

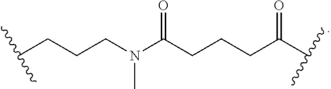 [SMILES: *CCCN(C)C(=O)CCCC(*)=O]

In some embodiments, L is

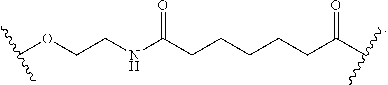 [SMILES: *OCCNC(=O)CCCCCC(*)=O]

In some embodiments, L is

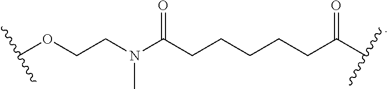 [SMILES: *OCCN(C)C(=O)CCCCCC(*)=O]

In some embodiments, L is

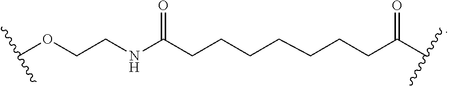 [SMILES: *OCCNC(=O)CCCCCCCC(*)=O]

In some embodiments, L is

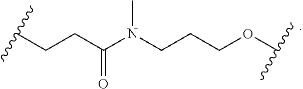 [SMILES: *CCC(=O)N(C)CCCO*]

In some embodiments, L is

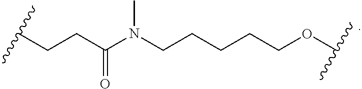 [SMILES: *CCC(=O)N(C)CCCCCO*]

In some embodiments, L is

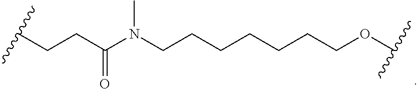 [SMILES: *CCC(=O)N(C)CCCCCCCO*]

In some embodiments, L is

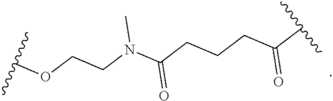 [SMILES: *OCCN(C)C(=O)CCCC(*)=O]

In some embodiments, L is

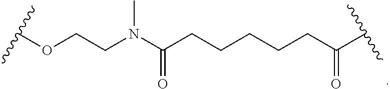 [SMILES: *OCCN(C)C(=O)CCCCCC(*)=O]

In some embodiments, L is

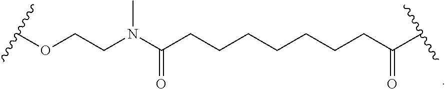 [SMILES: *OCCN(C)C(=O)CCCCCCCC(*)=O]

In some embodiments, L is

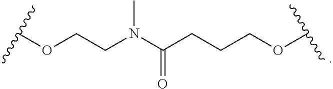 [SMILES: *OCCCC(=O)N(C)CCO*]

In some embodiments, L is

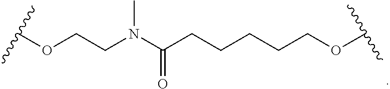 [SMILES: *OCCCCCC(=O)N(C)CCO*]

In some embodiments, L is

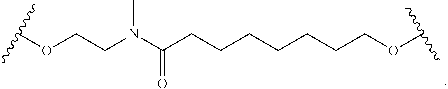 [SMILES: *OCCCCCCCC(=O)N(C)CCO*]

In some embodiments, L is

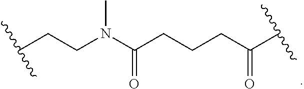 [SMILES: *CCN(C)C(=O)CCCC(*)=O]

In some embodiments, L is

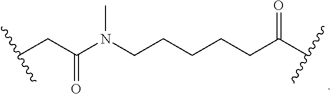 [SMILES: *CC(=O)N(C)CCCCCC(*)=O]

In some embodiments, L is

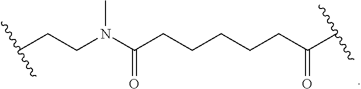 [SMILES: *CCN(C)C(=O)CCCCCC(*)=O]

In some embodiments, L is

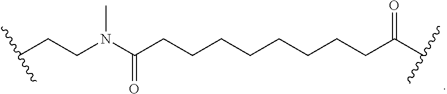 [SMILES: *CCN(C)C(=O)CCCCCCCCC(*)=O]

In some embodiments, L is

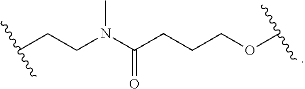 [SMILES: *CCN(C)C(=O)CCCO*]

In some embodiments, L is

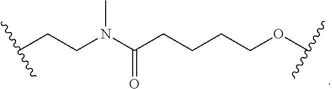 [SMILES: *CCN(C)C(=O)CCCCO*]

In some embodiments, L is

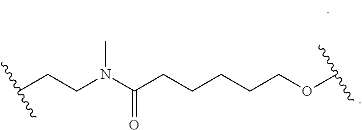 [SMILES: *CCN(C)C(=O)CCCCCO*]

In some embodiments, L is

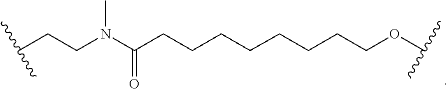 [SMILES: *CCN(C)C(=O)CCCCCCCCO*]

In some embodiments, L is

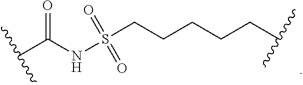 [SMILES: *CCCCCS(=O)(=O)NC(*)=O]

In some embodiments, L is

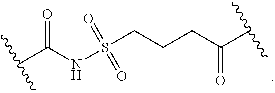 [SMILES: *C(=O)CCCS(=O)(=O)NC(*)=O]

In some embodiments, L is

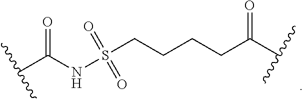 [SMILES: *C(=O)CCCCS(=O)(=O)NC(*)=O]

In some embodiments, L is

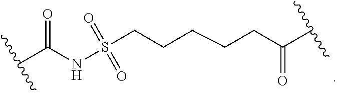 [SMILES: *C(=O)CCCCCS(=O)(=O)NC(*)=O]

In some embodiments, L is

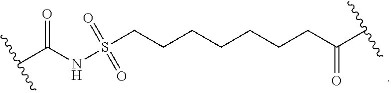 [SMILES: *C(=O)CCCCCCCS(=O)(=O)NC(*)=O]

In some embodiments, L is

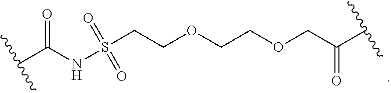 [SMILES: *C(=O)COCCOCCS(=O)(=O)NC(*)=O]

In some embodiments, L is

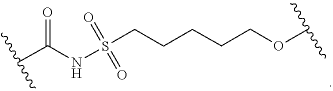 [SMILES: *OCCCCCS(=O)(=O)NC(*)=O]

In some embodiments, L is

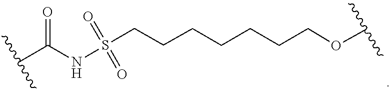 [SMILES: *OCCCCCCCS(=O)(=O)NC(*)=O]

In some embodiments, L is

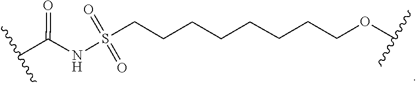 [SMILES: *OCCCCCCCCS(=O)(=O)NC(*)=O]

In some embodiments, L is

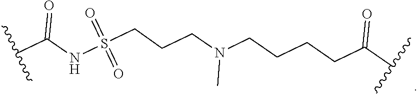 [SMILES: *C(=O)CCCCN(C)CCCS(=O)(=O)NC(*)=O]

In some embodiments, L is

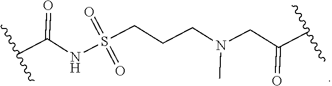 [SMILES: *C(=O)CN(C)CCCS(=O)(=O)NC(*)=O]

In some embodiments, L is

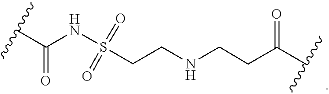 [SMILES: *C(=O)CCNCCS(=O)(=O)NC(*)=O]

In some embodiments, L is

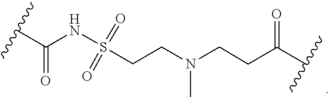 [SMILES: *C(=O)CCN(C)CCS(=O)(=O)NC(*)=O]

In some embodiments, L is

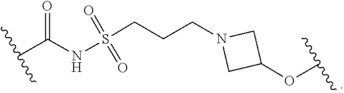 [SMILES: *OC1CN(CCCS(=O)(=O)NC(*)=O)C1]

In some embodiments, L is

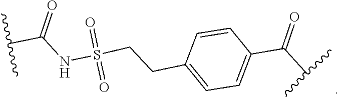 [SMILES: *C(=O)NS(=O)(=O)CCc1ccc(C(*)=O)cc1]

In some embodiments, L is

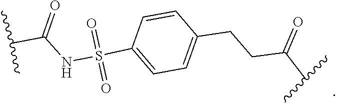 [SMILES: *C(=O)CCc1ccc(S(=O)(=O)NC(*)=O)cc1]

In some embodiments, L is

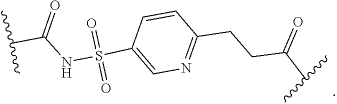 [SMILES: *C(=O)CCc1ccc(S(=O)(=O)NC(*)=O)cn1]

In some embodiments, L is

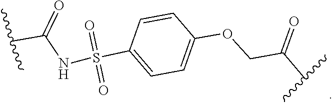 [SMILES: *C(=O)COc1ccc(S(=O)(=O)NC(*)=O)cc1]

In some embodiments, L is

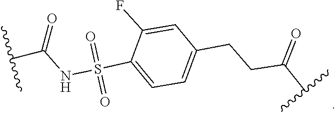 [SMILES: *C(=O)CCc1ccc(S(=O)(=O)NC(*)=O)c(F)c1]

In some embodiments, L is

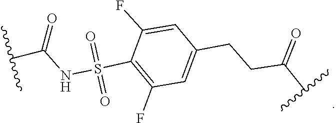 [SMILES: *C(=O)CCc1cc(F)c(S(=O)(=O)NC(*)=O)c(F)c1]

In some embodiments, L is

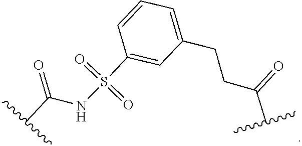 [SMILES: *C(=O)CCc1cccc(S(=O)(=O)NC(*)=O)c1]

In some embodiments, L is

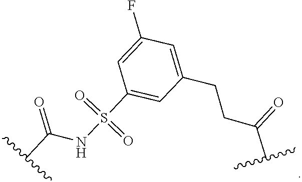 [SMILES: *C(=O)CCc1cc(F)cc(S(=O)(=O)NC(*)=O)c1]

In some embodiments, L is

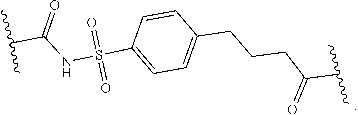 [SMILES: *C(=O)CCCc1ccc(S(=O)(=O)NC(*)=O)cc1]

In some embodiments, L is

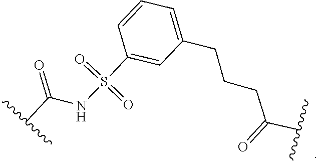 [SMILES: *C(=O)CCCc1cccc(S(=O)(=O)NC(*)=O)c1]

In some embodiments, L is

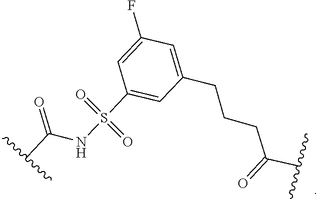 [SMILES: *C(=O)CCCc1cc(F)cc(S(=O)(=O)NC(*)=O)c1]

In some embodiments, L is

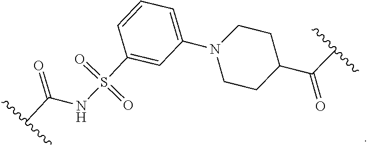 [SMILES: *C(=O)NS(=O)(=O)c1cccc(N2CCC(C(*)=O)CC2)c1]

In some embodiments, L is

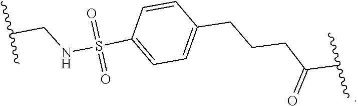 [SMILES: *CNS(=O)(=O)c1ccc(CCCC(*)=O)cc1]

In some embodiments, L is

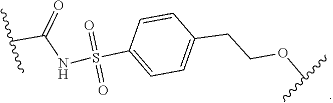 [SMILES: *OCCc1ccc(S(=O)(=O)NC(*)=O)cc1]

In some embodiments, L is

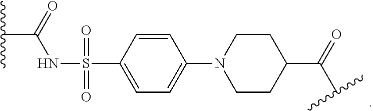 [SMILES: *C(=O)NS(=O)(=O)c1ccc(N2CCC(C(*)=O)CC2)cc1]

In some embodiments, L is

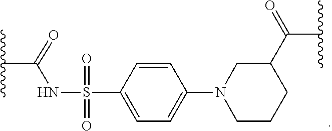 [SMILES: *C(=O)NS(=O)(=O)c1ccc(N2CCCC(C(*)=O)C2)cc1]

In some embodiments, L is

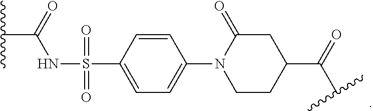 [SMILES: *C(=O)NS(=O)(=O)c1ccc(N2CCC(C(*)=O)CC2=O)cc1]

In some embodiments, L is

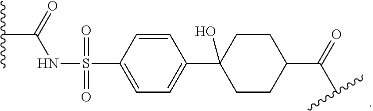 [SMILES: *C(=O)NS(=O)(=O)c1ccc(C2(O)CCC(C(*)=O)CC2)cc1]

In some embodiments, L is

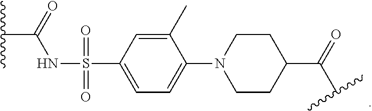 [SMILES: *C(=O)NS(=O)(=O)c1ccc(N2CCC(C(*)=O)CC2)c(C)c1]

In some embodiments, L is

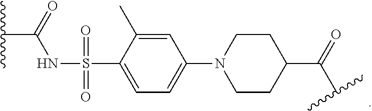 [SMILES: *C(=O)NS(=O)(=O)c1ccc(N2CCC(C(*)=O)CC2)cc1C]

In some embodiments, L is

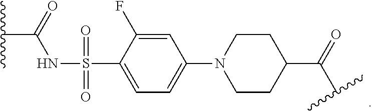 [SMILES: *C(=O)NS(=O)(=O)c1ccc(N2CCC(C(*)=O)CC2)cc1F]

In some embodiments, L is

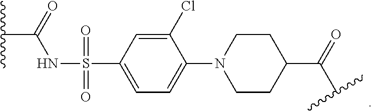 [SMILES: *C(=O)NS(=O)(=O)c1ccc(N2CCC(C(*)=O)CC2)c(Cl)c1]

In some embodiments, L is

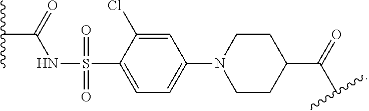 [SMILES: *C(=O)NS(=O)(=O)c1ccc(N2CCC(C(*)=O)CC2)cc1Cl]

In some embodiments, L is

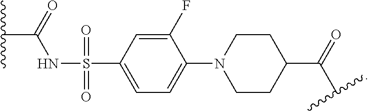 [SMILES: *C(=O)NS(=O)(=O)c1ccc(N2CCC(C(*)=O)CC2)c(F)c1]

In some embodiments, L is

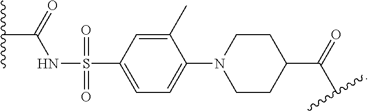 [SMILES: *C(=O)NS(=O)(=O)c1ccc(N2CCC(C(*)=O)CC2)c(C)c1]

In some embodiments, L is

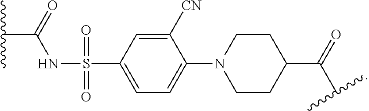 [SMILES: *C(=O)NS(=O)(=O)c1ccc(N2CCC(C(*)=O)CC2)c(C#N)c1]

In some embodiments, L is

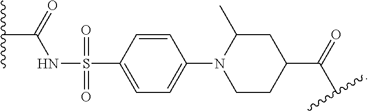 [SMILES: *C(=O)NS(=O)(=O)c1ccc(N2CCC(C(*)=O)CC2C)cc1]

In some embodiments, L is

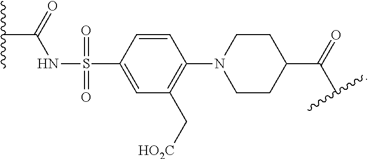 [SMILES: *C(=O)NS(=O)(=O)c1ccc(N2CCC(C(*)=O)CC2)c(CC(=O)O)c1]

In some embodiments, L is

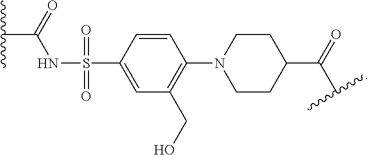 [SMILES: *C(=O)NS(=O)(=O)c1ccc(N2CCC(C(*)=O)CC2)c(CO)c1]

In some embodiments, L is

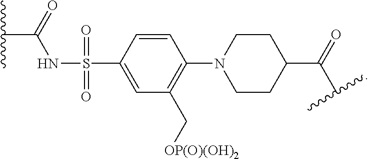

In some embodiments, L is

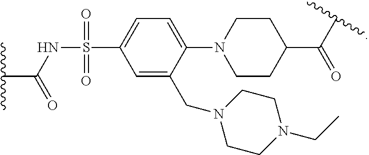 [SMILES: *C(=O)NS(=O)(=O)c1ccc(N2CCC(C(*)=O)CC2)c(CN2CCN(CC)CC2)c1]

In some embodiments, L is

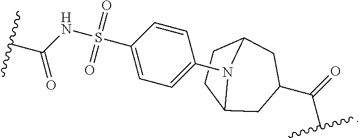 [SMILES: *C(=O)NS(=O)(=O)c1ccc(N2C3CCC2CC(C(*)=O)C3)cc1]

In some embodiments, L is

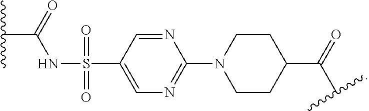 [SMILES: *C(=O)NS(=O)(=O)c1cnc(N2CCC(C(*)=O)CC2)nc1]

In some embodiments, L is

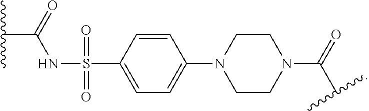 [SMILES: *C(=O)NS(=O)(=O)c1ccc(N2CCN(C(*)=O)CC2)cc1]

In some embodiments, L is

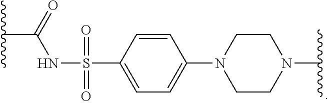 [SMILES: *C(=O)NS(=O)(=O)c1ccc(N2CCN(*)CC2)cc1]

In some embodiments, L is

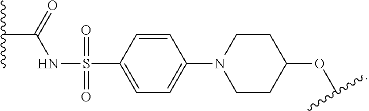 [SMILES: *OC1CCN(c2ccc(S(=O)(=O)NC(*)=O)cc2)CC1]

In some embodiments, L is

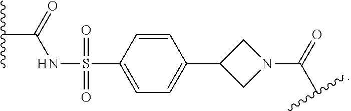 [SMILES: *C(=O)NS(=O)(=O)c1ccc(C2CN(C(*)=O)C2)cc1]

In some embodiments, L is

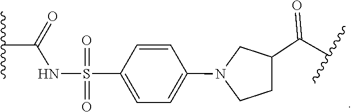 [SMILES: *C(=O)NS(=O)(=O)c1ccc(N2CCC(C(*)=O)C2)cc1]

In some embodiments, L is

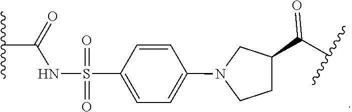 [SMILES: *C(=O)NS(=O)(=O)c1ccc(N2CC[C@H](C(*)=O)C2)cc1]

In some embodiments, L is

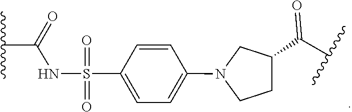 [SMILES: *C(=O)NS(=O)(=O)c1ccc(N2CC[C@@H](C(*)=O)C2)cc1]

In some embodiments, L is

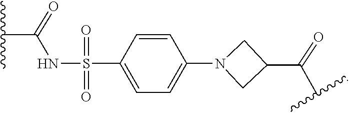 [SMILES: *C(=O)NS(=O)(=O)c1ccc(N2CC(C(*)=O)C2)cc1]

In some embodiments, L is

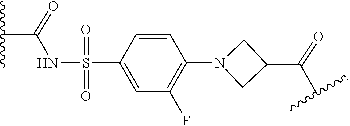 [SMILES: *C(=O)NS(=O)(=O)c1ccc(N2CC(C(*)=O)C2)c(F)c1]

In some embodiments, L is

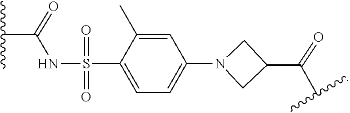 [SMILES: *C(=O)NS(=O)(=O)c1ccc(N2CC(C(*)=O)C2)cc1C]

In some embodiments, L is

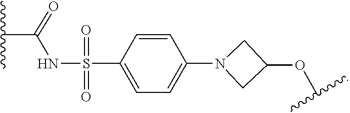 [SMILES: *OC1CN(c2ccc(S(=O)(=O)NC(*)=O)cc2)C1]

In some embodiments, L is

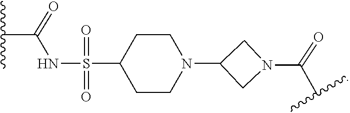 [SMILES: *C(=O)NS(=O)(=O)C1CCN(C2CN(C(*)=O)C2)CC1]

In some embodiments, L is

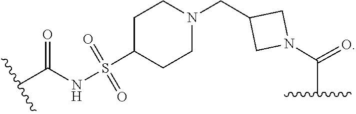 [SMILES: *C(=O)NS(=O)(=O)C1CCN(CC2CN(C(*)=O)C2)CC1]

In some embodiments, L is

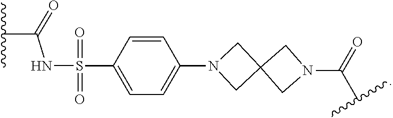 [SMILES: *C(=O)NS(=O)(=O)c1ccc(N2CC3(CN(C(*)=O)C3)C2)cc1]

In some embodiments, L is

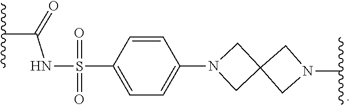 [SMILES: *C(=O)NS(=O)(=O)c1ccc(N2CC3(CN(*)C3)C2)cc1]

In some embodiments, L is

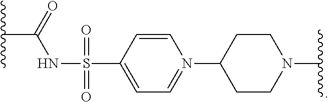

In some embodiments, L is

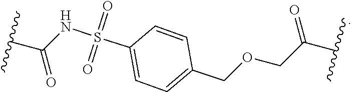 [SMILES: *C(=O)COCc1ccc(S(=O)(=O)NC(*)=O)cc1]

In some embodiments, L is

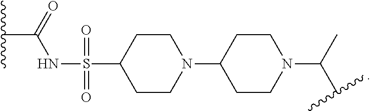 [SMILES: *C(=O)NS(=O)(=O)C1CCN(C2CCN(C(*)C)CC2)CC1]

In some embodiments, L is

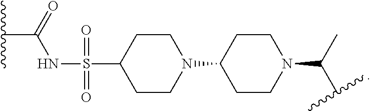

In some embodiments, L is

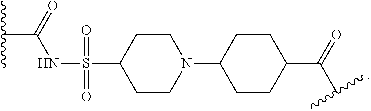 [SMILES: *C(=O)NS(=O)(=O)C1CCN(C2CCC(C(*)=O)CC2)CC1]

In some embodiments, L is

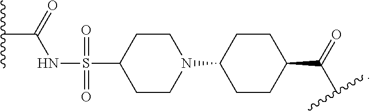 [SMILES: *C(=O)NS(=O)(=O)C1CCN([C@H]2CC[C@H](C(*)=O)CC2)CC1]

In some embodiments, L is

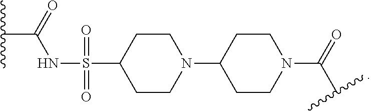 [SMILES: *C(=O)NS(=O)(=O)C1CCN(C2CCN(C(*)=O)CC2)CC1]

In some embodiments, L is

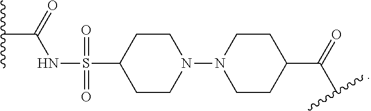 [SMILES: *C(=O)NS(=O)(=O)C1CCN(N2CCC(C(*)=O)CC2)CC1]

In some embodiments, L is

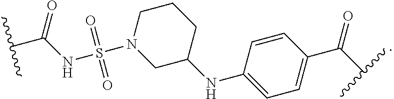 [SMILES: *C(=O)NS(=O)(=O)N1CCCC(Nc2ccc(C(*)=O)cc2)C1]

In some embodiments, L is

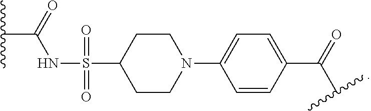 [SMILES: *C(=O)NS(=O)(=O)C1CCN(c2ccc(C(*)=O)cc2)CC1]

In some embodiments, L is

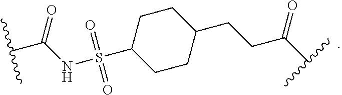 [SMILES: *C(=O)CCC1CCC(S(=O)(=O)NC(*)=O)CC1]

In some embodiments, L is

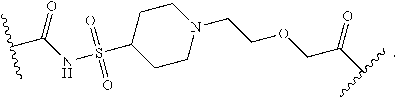 [SMILES: *C(=O)COCCN1CCC(S(=O)(=O)NC(*)=O)CC1]

In some embodiments, L is

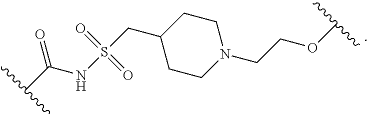 [SMILES: *OCCN1CCC(CS(=O)(=O)NC(*)=O)CC1]

In some embodiments, L is

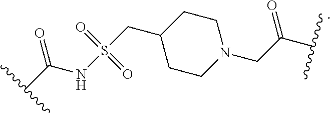 [SMILES: *C(=O)CN1CCC(CS(=O)(=O)NC(*)=O)CC1]

In some embodiments, L is

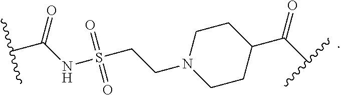 [SMILES: *C(=O)NS(=O)(=O)CCN1CCC(C(*)=O)CC1]

In some embodiments, L is

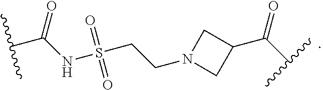 [SMILES: *C(=O)NS(=O)(=O)CCN1CC(C(*)=O)C1]

In some embodiments, L is

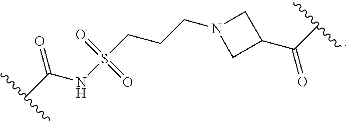 [SMILES: *C(=O)NS(=O)(=O)CCCN1CC(C(*)=O)C1]

In some embodiments, L is

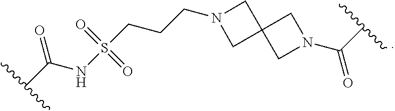 [SMILES: *C(=O)NS(=O)(=O)CCCN1CC2(C1)CN(C(*)=O)C2]

In some embodiments, L is

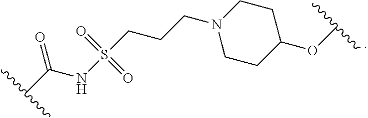 [SMILES: *OC1CCN(CCCS(=O)(=O)NC(*)=O)CC1]

In some embodiments, L is

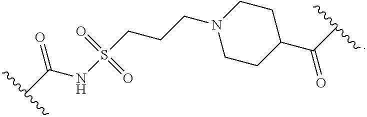 [SMILES: *C(=O)NS(=O)(=O)CCCN1CCC(C(*)=O)CC1]

In some embodiments, L is

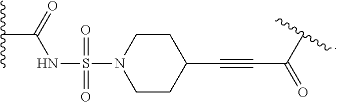 [SMILES: *C(=O)C#CC1CCN(S(=O)(=O)NC(*)=O)CC1]

In some embodiments, L is

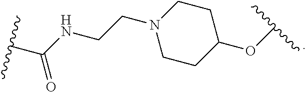 [SMILES: *OC1CCN(CCNC(*)=O)CC1]

In some embodiments, L is

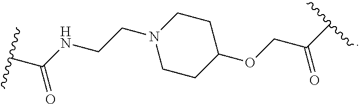 [SMILES: *C(=O)COC1CCN(CCNC(*)=O)CC1]

In some embodiments, L is

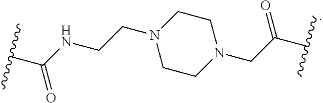 [SMILES: *C(=O)CN1CCN(CCNC(*)=O)CC1]

In some embodiments, L is

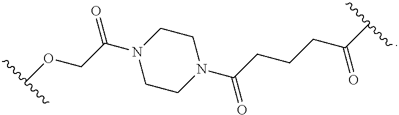 [SMILES: *OCC(=O)N1CCN(C(=O)CCCC(*)=O)CC1]

In some embodiments, L is

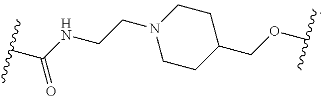 [SMILES: *OCC1CCN(CCNC(*)=O)CC1]

In some embodiments, L is

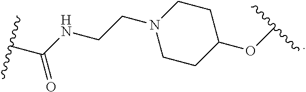 [SMILES: *OC1CCN(CCNC(*)=O)CC1]

In some embodiments, L is

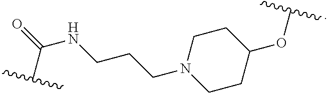 [SMILES: *OC1CCN(CCCNC(*)=O)CC1]

In some embodiments, L is

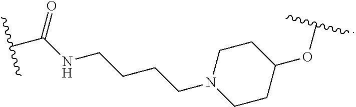 [SMILES: *OC1CCN(CCCCNC(*)=O)CC1]

In some embodiments, L is

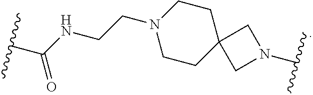 [SMILES: *C(=O)NCCN1CCC2(CC1)CN(*)C2]

In some embodiments, L is

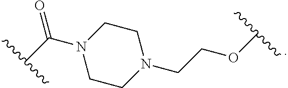 [SMILES: *OCCN1CCN(C(*)=O)CC1]

In some embodiments, L is

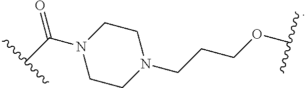 [SMILES: *OCCCN1CCN(C(*)=O)CC1]

In some embodiments, L is

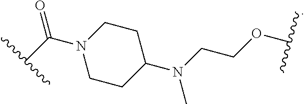 [SMILES: *OCCN(C)C1CCN(C(*)=O)CC1]

In some embodiments, L is

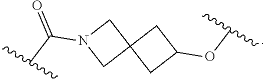 [SMILES: *OC1CC2(C1)CN(C(*)=O)C2]

In some embodiments, L is

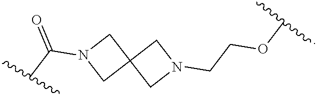 [SMILES: *OCCN1CC2(C1)CN(C(*)=O)C2]

In some embodiments, L is

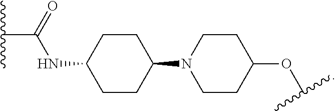 [SMILES: *OC1CCN([C@H]2CC[C@H](NC(*)=O)CC2)CC1]

In some embodiments, L is

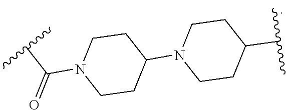 [SMILES: *C(=O)N1CCC(N2CCC(*)CC2)CC1]

In some embodiments, L is

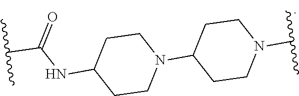 [SMILES: *C(=O)NC1CCN(C2CCN(*)CC2)CC1]

In some embodiments, L is

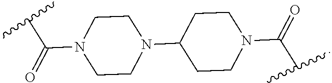 [SMILES: *C(=O)N1CCC(N2CCN(C(*)=O)CC2)CC1]

In some embodiments, L is

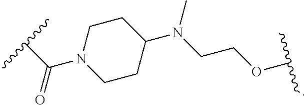 [SMILES: *OCCN(C)C1CCN(C(*)=O)CC1]

In some embodiments, L is

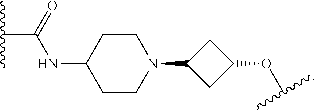

In some embodiments, L is

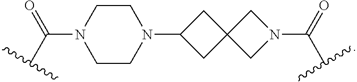 [SMILES: *C(=O)N1CCN(C2CC3(C2)CN(C(*)=O)C3)CC1]

In some embodiments, L is

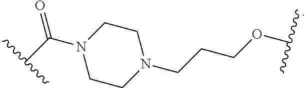 [SMILES: *OCCCN1CCN(C(*)=O)CC1]

In some embodiments, L is

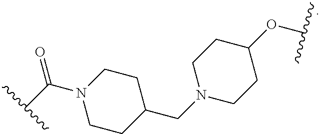 [SMILES: *OC1CCN(CC2CCN(C(*)=O)CC2)CC1]

In some embodiments, L is

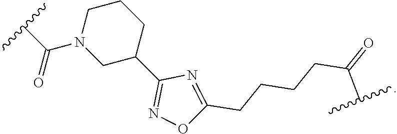 [SMILES: *C(=O)CCCCc1nc(C2CCCN(C(*)=O)C2)no1]

In some embodiments, L is

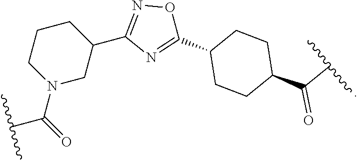 [SMILES: *C(=O)N1CCCC(c2noc([C@H]3CC[C@H](C(*)=O)CC3)n2)C1]

In some embodiments, L is

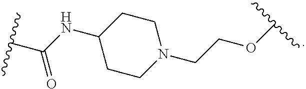 [SMILES: *OCCN1CCC(NC(*)=O)CC1]

In some embodiments, L is

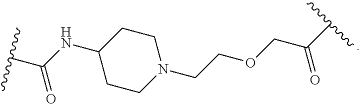 [SMILES: *C(=O)COCCN1CCC(NC(*)=O)CC1]

In some embodiments, L is

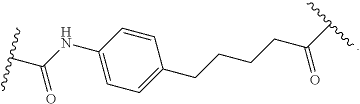 [SMILES: *C(=O)CCCCc1ccc(NC(*)=O)cc1]

In some embodiments, L is

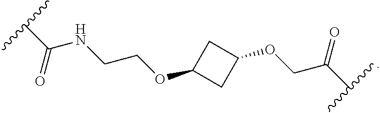 [SMILES: *C(=O)CO[C@H]1C[C@H](OCCNC(*)=O)C1]

In some embodiments, L is

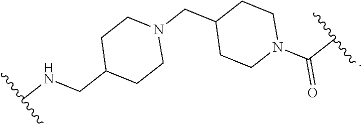 [SMILES: *NCC1CCN(CC2CCN(C(*)=O)CC2)CC1]

In some embodiments, L is

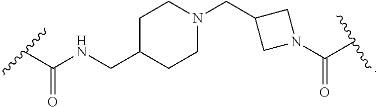 [SMILES: *C(=O)NCC1CCN(CC2CN(C(*)=O)C2)CC1]

In some embodiments, L is

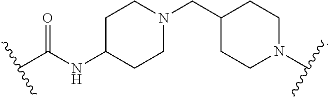 [SMILES: *C(=O)NC1CCN(CC2CCN(*)CC2)CC1]

In some embodiments, L is

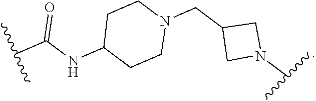 [SMILES: *C(=O)NC1CCN(CC2CN(*)C2)CC1]

In some embodiments, L is

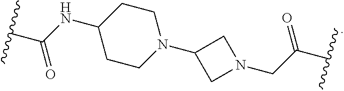 [SMILES: *C(=O)CN1CC(N2CCC(NC(*)=O)CC2)C1]

In some embodiments, L is

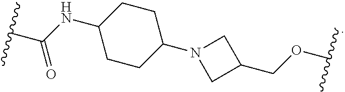 [SMILES: *OCC1CN(C2CCC(NC(*)=O)CC2)C1]

In some embodiments, L is

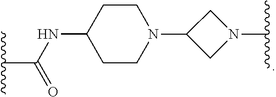 [SMILES: *C(=O)NC1CCN(C2CN(*)C2)CC1]

In some embodiments, L is

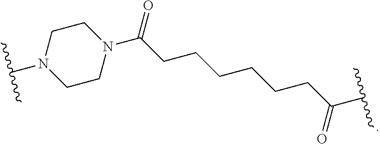 [SMILES: *C(=O)CCCCCCC(=O)N1CCN(*)CC1]

In some embodiments, L is

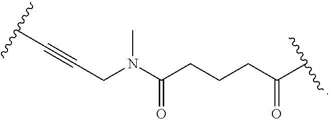 [SMILES: *C#CCN(C)C(=O)CCCC(*)=O]

In some embodiments, L is

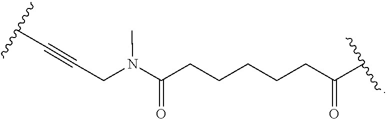 [SMILES: *C#CCN(C)C(=O)CCCCCC(*)=O]

In some embodiments, L is

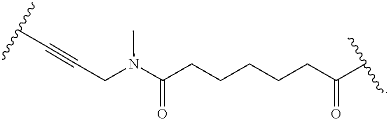 [SMILES: *C#CCN(C)C(=O)CCCCCC(*)=O]

In some embodiments, L is

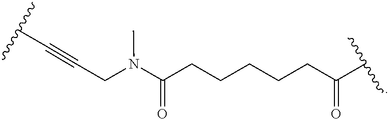 [SMILES: *C#CCN(C)C(=O)CCCCCC(*)=O]

In some embodiments, L is

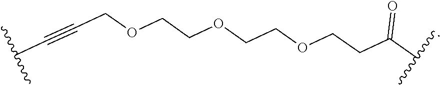 [SMILES: *C#CCOCCOCCOCCC(*)=O]

In some embodiments, L is

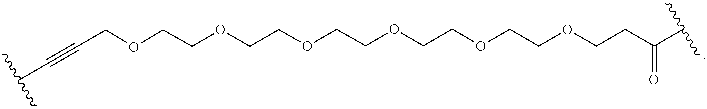 [SMILES: *C#CCOCCOCCOCCOCCOCCOCCC(*)=O]

In some embodiments, L is

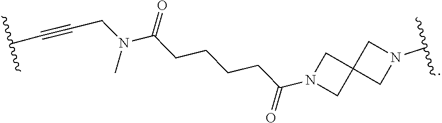 [SMILES: *C#CCN(C)C(=O)CCCCC(=O)N1CC2(CN(*)C2)C1]

In some embodiments, L is

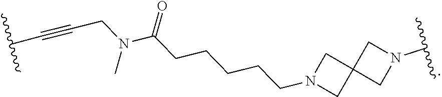 [SMILES: *C#CCN(C)C(=O)CCCCCN1CC2(CN(*)C2)C1]

In some embodiments, L is

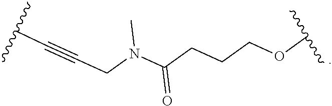 [SMILES: *C#CCN(C)C(=O)CCCO*]

In some embodiments, L is

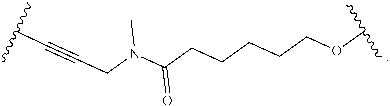 [SMILES: *C#CCN(C)C(=O)CCCCCO*]

In some embodiments, L is

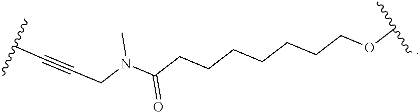 [SMILES: *C#CCN(C)C(=O)CCCCCCCO*]

In some embodiments, L is

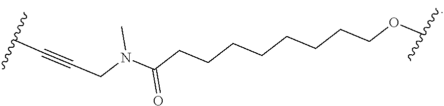 [SMILES: *C#CCN(C)C(=O)CCCCCCCCO*]

In some embodiments, L is

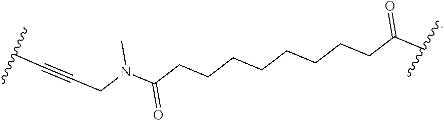 [SMILES: *C#CCN(C)C(=O)CCCCCCCCC(*)=O]

In some embodiments, L is

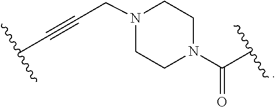 [SMILES: *C#CCN1CCN(C(*)=O)CC1]

In some embodiments, L is

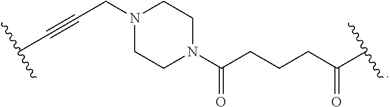 [SMILES: *C#CCN1CCN(C(=O)CCCC(*)=O)CC1]

In some embodiments, L is

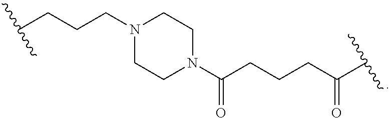 [SMILES: *CCCN1CCN(C(=O)CCCC(*)=O)CC1]

In some embodiments, L is

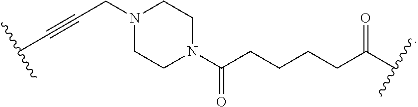 [SMILES: *C#CCN1CCN(C(=O)CCCCC(*)=O)CC1]

In some embodiments, L is

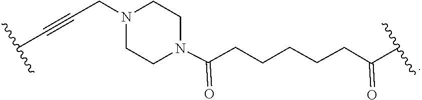 [SMILES: *C#CCN1CCN(C(=O)CCCCCC(*)=O)CC1]

In some embodiments, L is

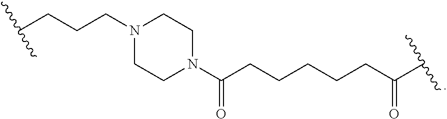 [SMILES: *CCCN1CCN(C(=O)CCCCCC(*)=O)CC1]

In some embodiments, L is

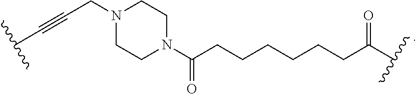 [SMILES: *C#CCN1CCN(C(=O)CCCCCCC(*)=O)CC1]

In some embodiments, L is

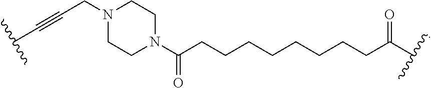 [SMILES: *C#CCN1CCN(C(=O)CCCCCCCCC(*)=O)CC1]

In some embodiments, L is

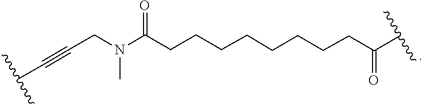 [SMILES: *C#CCN(C)C(=O)CCCCCCCCC(*)=O]

In some embodiments, L is

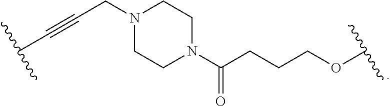 [SMILES: *C#CCN1CCN(C(=O)CCCO*)CC1]

In some embodiments, L is

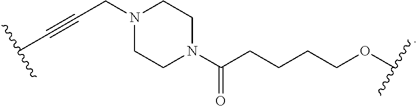 [SMILES: *C#CCN1CCN(C(=O)CCCCO*)CC1]

In some embodiments, L is

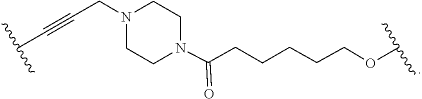 [SMILES: *C#CCN1CCN(C(=O)CCCCCO*)CC1]

In some embodiments, L is

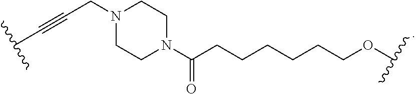 [SMILES: *C#CCN1CCN(C(=O)CCCCCCO*)CC1]

In some embodiments, L is

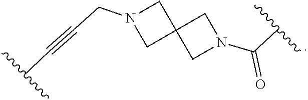 [SMILES: *C#CCN1CC2(C1)CN(C(*)=O)C2]

In some embodiments, L is

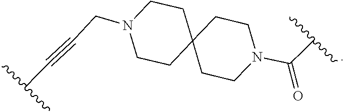 [SMILES: *C#CCN1CCC2(CC1)CCN(C(*)=O)CC2]

In some embodiments, L is

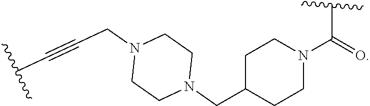 [SMILES: *C#CCN1CCN(CC2CCN(C(*)=O)CC2)CC1]

In some embodiments, L is

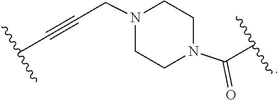 [SMILES: *C#CCN1CCN(C(*)=O)CC1]

In some embodiments, L is

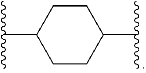 [SMILES: *C1CCC(*)CC1]

In some embodiments, L is

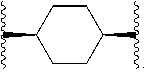 [SMILES: *[C@H]1CC[C@@H](*)CC1]

In some embodiments, L is

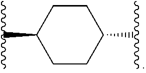 [SMILES: *[C@H]1CC[C@H](*)CC1]

In some embodiments, L is

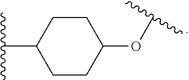 [SMILES: *OC1CCC(*)CC1]

In some embodiments, L is

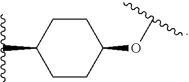 [SMILES: *O[C@H]1CC[C@@H](*)CC1]

In some embodiments, L is

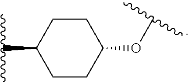 [SMILES: *O[C@H]1CC[C@H](*)CC1]

In some embodiments, L is

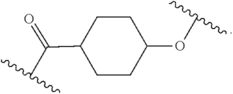 [SMILES: *OC1CCC(C(*)=O)CC1]

In some embodiments, L is

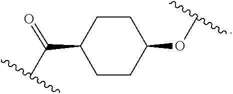 [SMILES: *O[C@H]1CC[C@@H](C(*)=O)CC1]

In some embodiments, L is

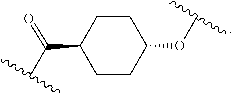 [SMILES: *O[C@H]1CC[C@H](C(*)=O)CC1]

In some embodiments, L is

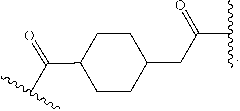 [SMILES: *C(=O)CC1CCC(C(*)=O)CC1]

In some embodiments, L is

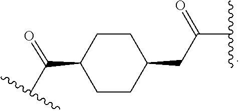 [SMILES: *C(=O)C[C@H]1CC[C@@H](C(*)=O)CC1]

In some embodiments, L is

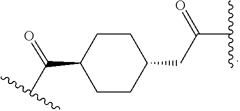 [SMILES: *C(=O)C[C@H]1CC[C@H](C(*)=O)CC1]

In some embodiments, L is

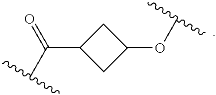 [SMILES: *OC1CC(C(*)=O)C1]

In some embodiments, L is

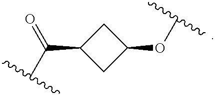 [SMILES: *O[C@H]1C[C@@H](C(*)=O)C1]

In some embodiments, L is

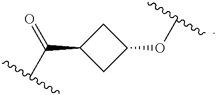 [SMILES: *O[C@H]1C[C@H](C(*)=O)C1]

In some embodiments, L is

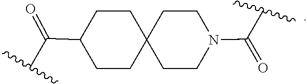 [SMILES: *C(=O)C1CCC2(CC1)CCN(C(*)=O)CC2]

In some embodiments, L is

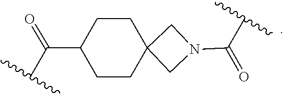 [SMILES: *C(=O)C1CCC2(CC1)CN(C(*)=O)C2]

In some embodiments, L is selected from those depicted in Table 1, below.

In some embodiments, L is selected from those depicted in Table B, below.

Without limitation, the point of attachment of L to BBM and DIM can be, for example when L is

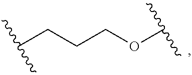 [SMILES: *CCCO*]

either

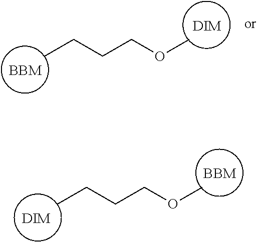

In some embodiments, a provided compound or pharmaceutically acceptable salt thereof, is selected from those wherein BBM is

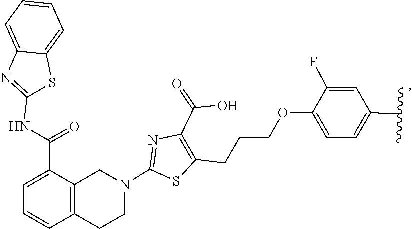 [SMILES: *c1ccc(OCCCc2sc(N3CCc4cccc(C(=O)Nc5nc6ccccc6s5)c4C3)nc2C(=O)O)c(F)c1]

LBM is selected from any of those in Table A below, and L is selected from any of those in Table B below.

In some embodiments, a provided compound or pharmaceutically acceptable salt thereof, is selected from those wherein BBM is

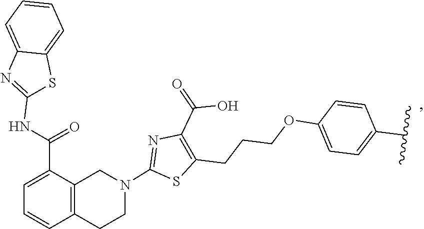 [SMILES: *c1ccc(OCCCc2sc(N3CCc4cccc(C(=O)Nc5nc6ccccc6s5)c4C3)nc2C(=O)O)cc1]

LBM is selected from any of those in Table A below, and L is selected from any of those in Table B below.

In some embodiments, a provided compound or pharmaceutically acceptable salt thereof, is selected from those wherein BBM is

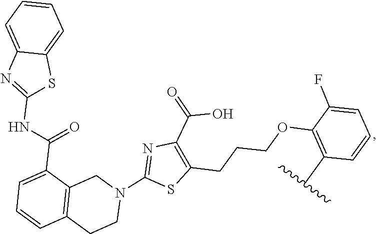 [SMILES: *c1cccc(F)c1OCCCc1sc(N2CCc3cccc(C(=O)Nc4nc5ccccc5s4)c3C2)nc1C(=O)O]

LBM is selected from any of those in Table A below, and L is selected from any of those in Table B below.

In some embodiments, a provided compound or pharmaceutically acceptable salt thereof, is selected from those wherein BBM is

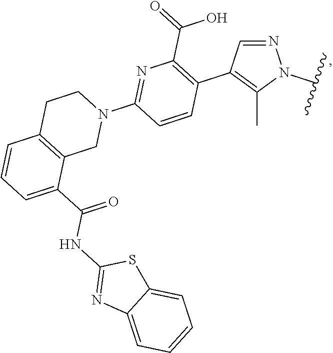 [SMILES: *n1ncc(-c2ccc(N3CCc4cccc(C(=O)Nc5nc6ccccc6s5)c4C3)nc2C(=O)O)c1C]

LBM is selected from any of those in Table A below, and L is selected from any of those in Table B below.

In some embodiments, a provided compound or pharmaceutically acceptable salt thereof, is selected from those wherein BBM is

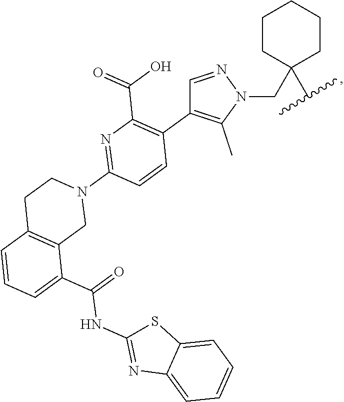 [SMILES: *C1(Cn2ncc(-c3ccc(N4CCc5cccc(C(=O)Nc6nc7ccccc7s6)c5C4)nc3C(=O)O)c2C)CCCCC1]

LBM is selected from any of those in Table A below, and L is selected from any of those in Table B below.

In some embodiments, a provided compound or pharmaceutically acceptable salt thereof, is selected from those wherein BBM is

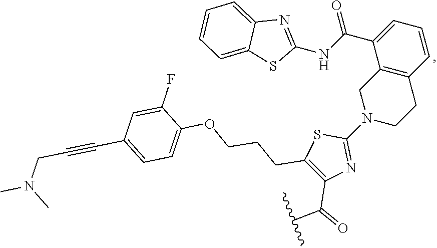 [SMILES: *C(=O)c1nc(N2CCc3cccc(C(=O)Nc4nc5ccccc5s4)c3C2)sc1CCCOc1ccc(C#CCN(C)C)cc1F]

LBM is selected from any of those in Table A below, and L is selected from any of those in Table B below.

In some embodiments, a provided compound or pharmaceutically acceptable salt thereof, is selected from those wherein BBM is

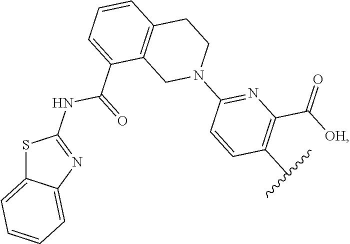 [SMILES: *c1ccc(N2CCc3cccc(C(=O)Nc4nc5ccccc5s4)c3C2)nc1C(=O)O]

LBM is selected from any of those in Table A below, and L is selected from any of those in Table B below.

In some embodiments, a provided compound or pharmaceutically acceptable salt thereof, is selected from those wherein BBM is

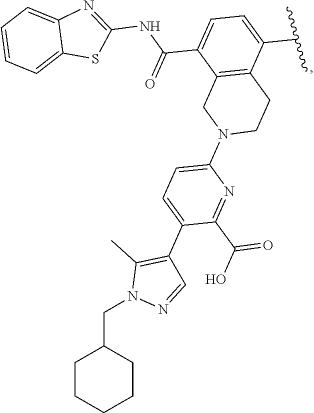 [SMILES: *c1ccc(C(=O)Nc2nc3ccccc3s2)c2c1CCN(c1ccc(-c3cnn(CC4CCCCC4)c3C)c(C(=O)O)n1)C2]

LBM is selected from any of those in

Table A below, and L is selected from any of those in Table B below.

In some embodiments, a provided compound or pharmaceutically acceptable salt thereof, is selected from those wherein BBM is

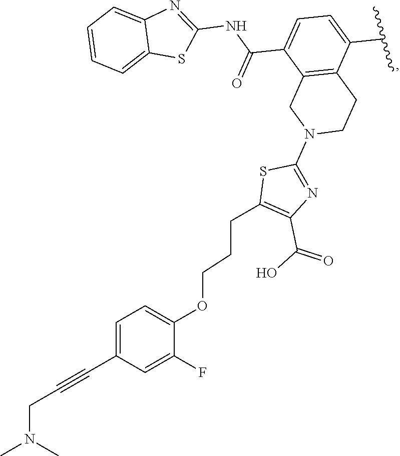 [SMILES: *c1ccc(C(=O)Nc2nc3ccccc3s2)c2c1CCN(c1nc(C(=O)O)c(CCCOc3ccc(C#CCN(C)C)cc3F)s1)C2]

LBM is selected from any of those in Table A below, and L is selected from any of those in Table B below.

In some embodiments, a provided compound or pharmaceutically acceptable salt thereof, is selected from those wherein BBM is

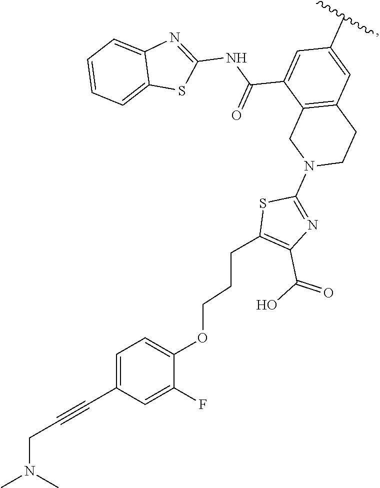 [SMILES: *c1cc2c(c(C(=O)Nc3nc4ccccc4s3)c1)CN(c1nc(C(=O)O)c(CCCOc3ccc(C#CCN(C)C)cc3F)s1)CC2]

LBM is selected from any of those in Table A below, and L is selected from any of those in Table B below.

In some embodiments, a provided compound or pharmaceutically acceptable salt thereof, is selected from those wherein BBM is

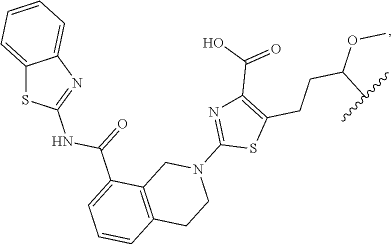 [SMILES: *C(CCc1sc(N2CCc3cccc(C(=O)Nc4nc5ccccc5s4)c3C2)nc1C(=O)O)OC]

LBM is selected from any of those in Table A below, and L is selected from any of those in Table B below.

In some embodiments, a provided compound or pharmaceutically acceptable salt thereof, is selected from those wherein BBM is

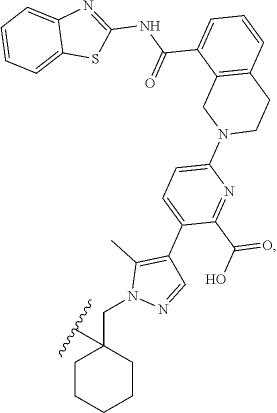 [SMILES: *C1(Cn2ncc(-c3ccc(N4CCc5cccc(C(=O)Nc6nc7ccccc7s6)c5C4)nc3C(=O)O)c2C)CCCCC1]

LBM is selected from any of those in Table A below, and L is selected from any of those in Table B below.

In some embodiments, a provided compound or pharmaceutically acceptable salt thereof, is selected from those wherein BBM is

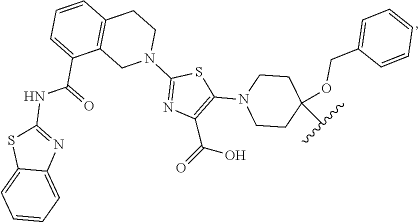 [SMILES: *C1(OCc2ccccc2)CCN(c2sc(N3CCc4cccc(C(=O)Nc5nc6ccccc6s5)c4C3)nc2C(=O)O)CC1]

LBM is selected from any of those in Table A below, and L is selected from any of those in Table B below.

In some embodiments, a provided compound or pharmaceutically acceptable salt thereof, is selected from those wherein BBM is

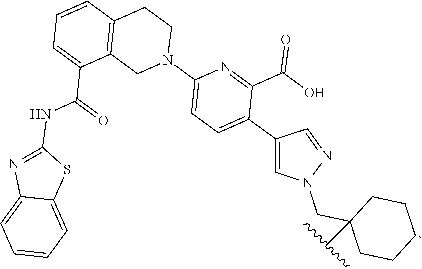 [SMILES: *C1(Cn2cc(-c3ccc(N4CCc5cccc(C(=O)Nc6nc7ccccc7s6)c5C4)nc3C(=O)O)cn2)CCCCC1]

LBM is selected from any of those in Table A below, and L is selected from any of those in Table B below.

In some embodiments, a provided compound or pharmaceutically acceptable salt thereof, is selected from those wherein BBM is

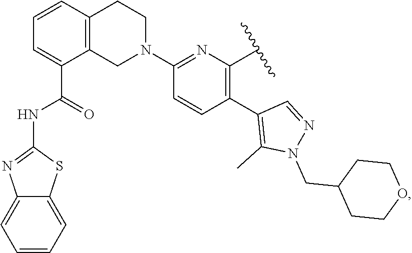 [SMILES: *c1nc(N2CCc3cccc(C(=O)Nc4nc5ccccc5s4)c3C2)ccc1-c1cnn(CC2CCOCC2)c1C]

LBM is selected from any of those in Table A below, and L is selected from any of those in Table B below.

In some embodiments, a provided compound or pharmaceutically acceptable salt thereof, is selected from those wherein BBM is

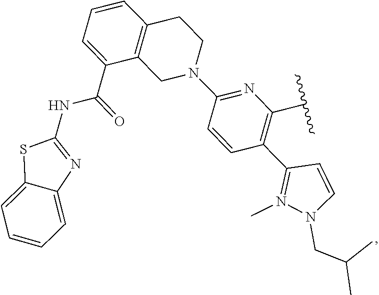

LBM is selected from any of those in Table A below, and L is selected from any of those in Table B below.

In some embodiments, a provided compound or pharmaceutically acceptable salt thereof, is selected from those wherein BBM is

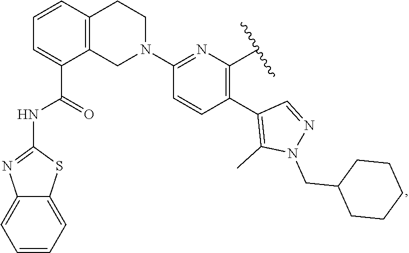 [SMILES: *c1nc(N2CCc3cccc(C(=O)Nc4nc5ccccc5s4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

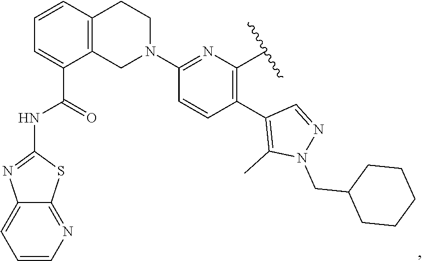 [SMILES: *c1nc(N2CCc3cccc(C(=O)Nc4nc5cccnc5s4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

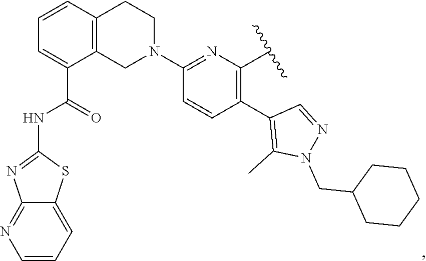 [SMILES: *c1nc(N2CCc3cccc(C(=O)Nc4nc5ncccc5s4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

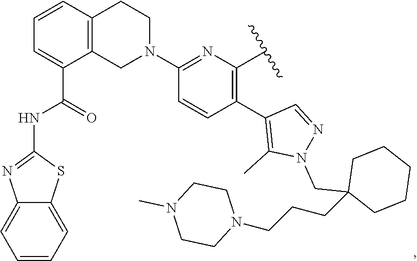 [SMILES: *c1nc(N2CCc3cccc(C(=O)Nc4nc5ccccc5s4)c3C2)ccc1-c1cnn(CC2(CCCN3CCN(C)CC3)CCCCC2)c1C]

LBM is selected from any of those in Table A below, and L is selected from any of those in Table B below.

In some embodiments, a provided compound or pharmaceutically acceptable salt thereof, is selected from those wherein BBM is

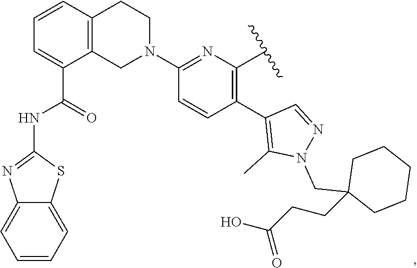 [SMILES: *c1nc(N2CCc3cccc(C(=O)Nc4nc5ccccc5s4)c3C2)ccc1-c1cnn(CC2(CCC(=O)O)CCCCC2)c1C]

LBM is selected from any of those in Table A below, and L is selected from any of those in Table B below.

In some embodiments, a provided compound or pharmaceutically acceptable salt thereof, is selected from those wherein BBM is

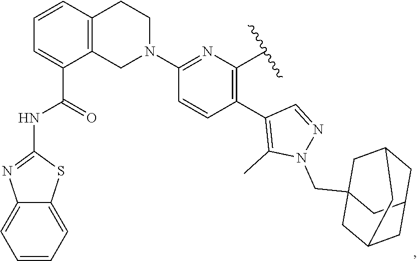 [SMILES: *c1nc(N2CCc3cccc(C(=O)Nc4nc5ccccc5s4)c3C2)ccc1-c1cnn(CC23CC4CC(CC(C4)C2)C3)c1C]

LBM is selected from any of those in Table A below, and L is selected from any of those in Table B below.

In some embodiments, a provided compound or pharmaceutically acceptable salt thereof, is selected from those wherein BBM is

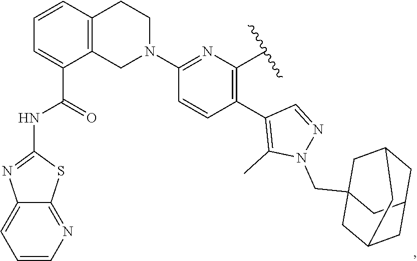 [SMILES: *c1nc(N2CCc3cccc(C(=O)Nc4nc5cccnc5s4)c3C2)ccc1-c1cnn(CC23CC4CC(CC(C4)C2)C3)c1C]

LBM is selected from any of those in Table A below, and L is selected from any of those in Table B below.

In some embodiments, a provided compound or pharmaceutically acceptable salt thereof, is selected from those wherein BBM is

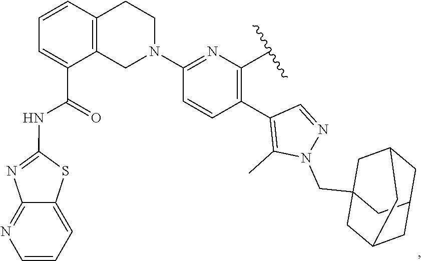 [SMILES: *c1nc(N2CCc3cccc(C(=O)Nc4nc5ncccc5s4)c3C2)ccc1-c1cnn(CC23CC4CC(CC(C4)C2)C3)c1C]

LBM is selected from any of those in Table A below, and L is selected from any of those in Table B below.

In some embodiments, a provided compound or pharmaceutically acceptable salt thereof, is selected from those wherein BBM is

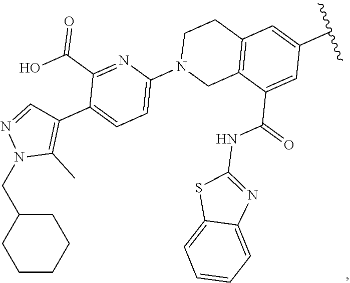 [SMILES: *c1cc2c(c(C(=O)Nc3nc4ccccc4s3)c1)CN(c1ccc(-c3cnn(CC4CCCCC4)c3C)c(C(=O)O)n1)CC2]

LBM is selected from any of those in Table A below, and L is selected from any of those in Table B below.

In some embodiments, a provided compound or pharmaceutically acceptable salt thereof, is selected from those wherein BBM is

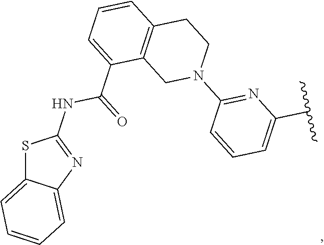 [SMILES: *c1cccc(N2CCc3cccc(C(=O)Nc4nc5ccccc5s4)c3C2)n1]

LBM is selected from any of those in Table A below, and L is selected from any of those in Table B below.

In some embodiments, a provided compound or pharmaceutically acceptable salt thereof, is selected from those wherein BBM is

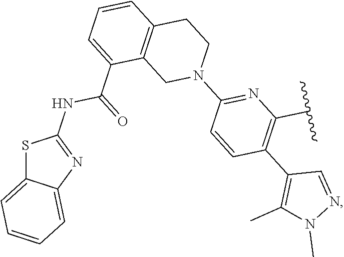 [SMILES: *c1nc(N2CCc3cccc(C(=O)Nc4nc5ccccc5s4)c3C2)ccc1-c1cnn(C)c1C]

LBM is selected from any of those in Table A below, and L is selected from any of those in Table B below.

In some embodiments, a provided compound or pharmaceutically acceptable salt thereof, is selected from those wherein BBM is

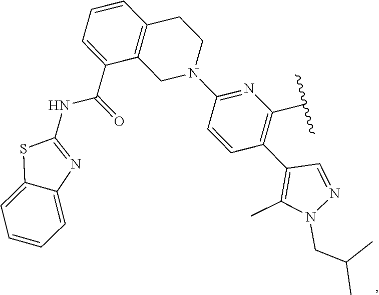 [SMILES: *c1nc(N2CCc3cccc(C(=O)Nc4nc5ccccc5s4)c3C2)ccc1-c1cnn(CC(C)C)c1C]

LBM is selected from any of those in Table A below, and L is selected from any of those in Table B below.

In some embodiments, a provided compound or pharmaceutically acceptable salt thereof, is selected from those wherein BBM is

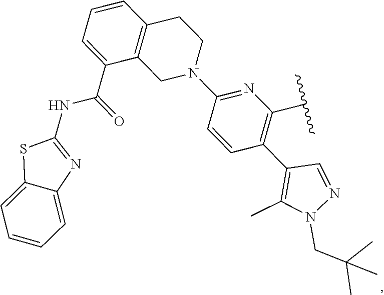 [SMILES: *c1nc(N2CCc3cccc(C(=O)Nc4nc5ccccc5s4)c3C2)ccc1-c1cnn(CC(C)(C)C)c1C]

LBM is selected from any of those in Table A below, and L is selected from any of those in Table B below.

In some embodiments, a provided compound or pharmaceutically acceptable salt thereof, is selected from those wherein BBM is

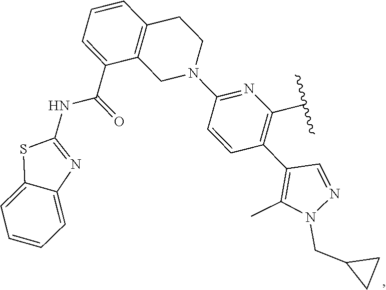 [SMILES: *c1nc(N2CCc3cccc(C(=O)Nc4nc5ccccc5s4)c3C2)ccc1-c1cnn(CC2CC2)c1C]

LBM is selected from any of those in Table A below, and L is selected from any of those in Table B below.

In some embodiments, a provided compound or pharmaceutically acceptable salt thereof, is selected from those wherein BBM is

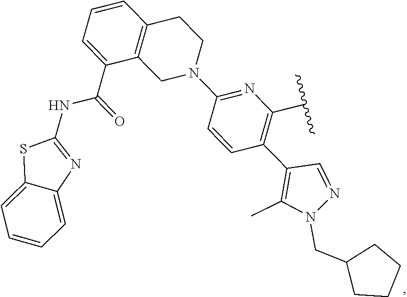 [SMILES: *c1nc(N2CCc3cccc(C(=O)Nc4nc5ccccc5s4)c3C2)ccc1-c1cnn(CC2CCCC2)c1C]

LBM is selected from any of those in Table A below, and L is selected from any of those in Table B below.

In some embodiments, a provided compound or pharmaceutically acceptable salt thereof, is selected from those wherein BBM is

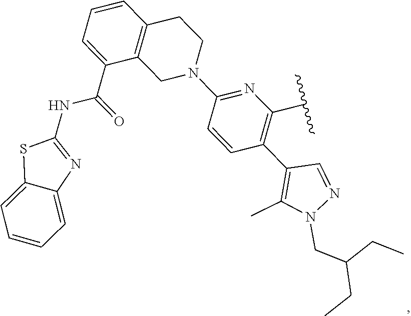 [SMILES: *c1nc(N2CCc3cccc(C(=O)Nc4nc5ccccc5s4)c3C2)ccc1-c1cnn(CC(CC)CC)c1C]

LBM is selected from any of those in Table A below, and L is selected from any of those in Table B below.

In some embodiments, a provided compound or pharmaceutically acceptable salt thereof, is selected from those wherein BBM is

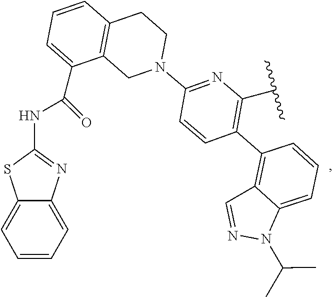 [SMILES: *c1nc(N2CCc3cccc(C(=O)Nc4nc5ccccc5s4)c3C2)ccc1-c1cccc2c1cnn2C(C)C]

LBM is selected from any of those in Table A below, and L is selected from any of those in Table B below.

In some embodiments, a provided compound or pharmaceutically acceptable salt thereof, is selected from those wherein BBM is

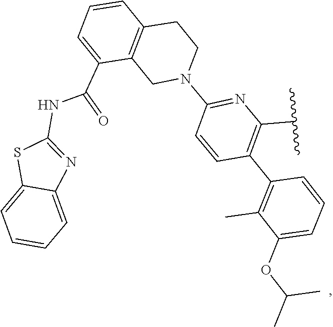 [SMILES: *c1nc(N2CCc3cccc(C(=O)Nc4nc5ccccc5s4)c3C2)ccc1-c1cccc(OC(C)C)c1C]

LBM is selected from any of those in Table A below, and L is selected from any of those in Table B below.

In some embodiments, a provided compound or pharmaceutically acceptable salt thereof, is selected from those wherein BBM is

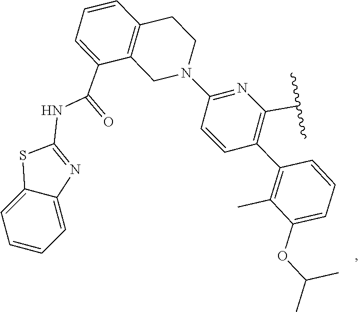 [SMILES: *c1nc(N2CCc3cccc(C(=O)Nc4nc5ccccc5s4)c3C2)ccc1-c1cccc(OC(C)C)c1C]

LBM is selected from any of those in Table A below, and L is selected from any of those in Table B below.

In some embodiments, a provided compound or pharmaceutically acceptable salt thereof, is selected from those wherein BBM is

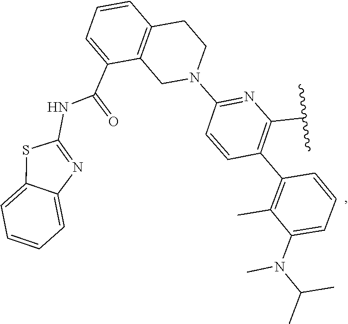 [SMILES: *c1nc(N2CCc3cccc(C(=O)Nc4nc5ccccc5s4)c3C2)ccc1-c1cccc(N(C)C(C)C)c1C]

LBM is selected from any of those in Table A below, and L is selected from any of those in Table B below.

In some embodiments, a provided compound or pharmaceutically acceptable salt thereof, is selected from those wherein BBM is

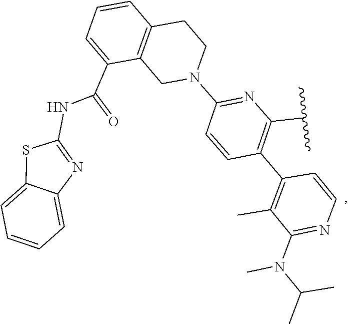 [SMILES: *c1nc(N2CCc3cccc(C(=O)Nc4nc5ccccc5s4)c3C2)ccc1-c1ccnc(N(C)C(C)C)c1C]

LBM is selected from any of those in Table A below, and L is selected from any of those in Table B below.

In some embodiments, a provided compound or pharmaceutically acceptable salt thereof, is selected from those wherein BBM is

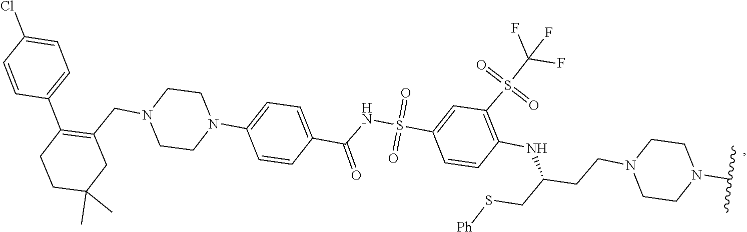 [SMILES: *N1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

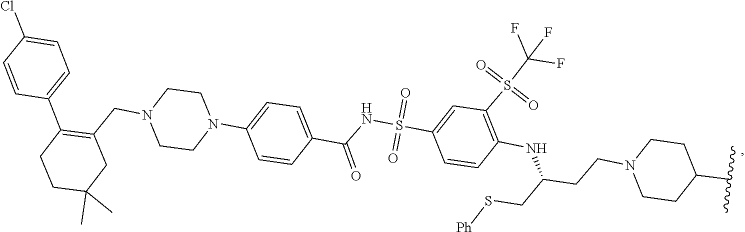 [SMILES: *C1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

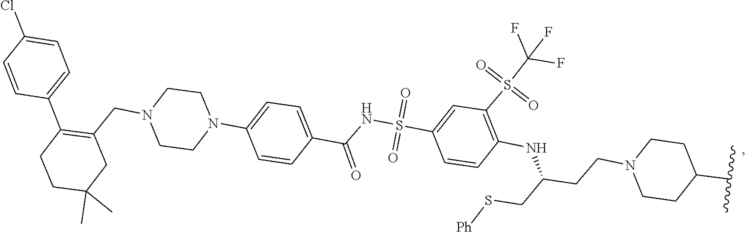 [SMILES: *C1CCN(CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

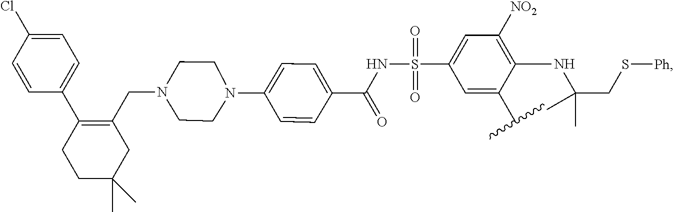 [SMILES: *c1cc(S(=O)(=O)NC(=O)c2ccc(N3CCN(CC4=C(c5ccc(Cl)cc5)CCC(C)(C)C4)CC3)cc2)cc([N+](=O)[O-])c1NC(C)(C)CSc1ccccc1]

LBM is selected from any of those in Table A below, and L is selected from any of those in Table B below.

In some embodiments, a provided compound or pharmaceutically acceptable salt thereof, is selected from those wherein BBM is

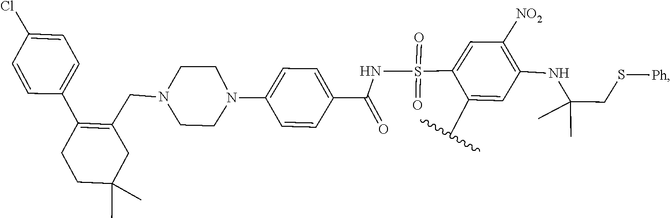 [SMILES: *c1cc(NC(C)(C)CSc2ccccc2)c([N+](=O)[O-])cc1S(=O)(=O)NC(=O)c1ccc(N2CCN(CC3=C(c4ccc(Cl)cc4)CCC(C)(C)C3)CC2)cc1]

LBM is selected from any of those in Table A below, and L is selected from any of those in Table B below.

In some embodiments, a provided compound or pharmaceutically acceptable salt thereof, is selected from those wherein BBM is

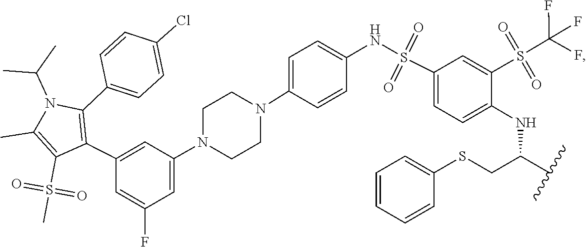 [SMILES: *[C@H](CSc1ccccc1)Nc1ccc(S(=O)(=O)Nc2ccc(N3CCN(c4cc(F)cc(-c5c(S(C)(=O)=O)c(C)n(C(C)C)c5-c5ccc(Cl)cc5)c4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is

selected from those wherein BBM is

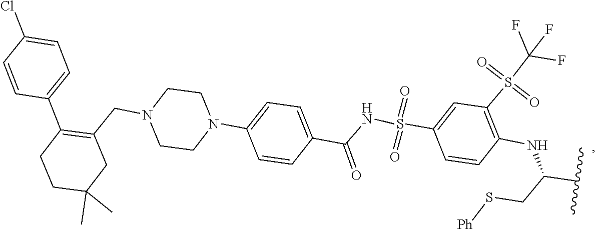 [SMILES: *[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

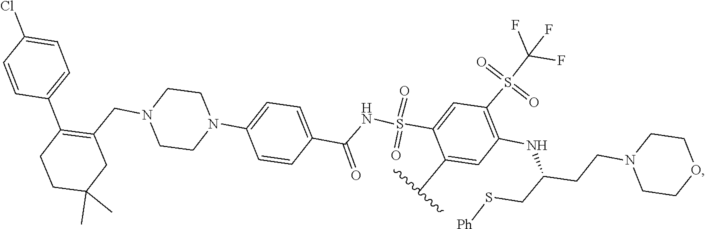 [SMILES: *c1cc(N[C@H](CCN2CCOCC2)CSc2ccccc2)c(S(=O)(=O)C(F)(F)F)cc1S(=O)(=O)NC(=O)c1ccc(N2CCN(CC3=C(c4ccc(Cl)cc4)CCC(C)(C)C3)CC2)cc1]

LBM is selected from any of those in Table A below, and L is selected from any of those in Table B below.

In some embodiments, a provided compound or pharmaceutically acceptable salt thereof, is selected from those wherein BBM is

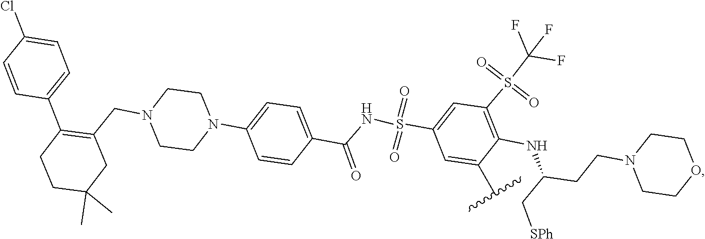

LBM is selected from any of those in Table A below, and L is selected from any of those in Table B below.

In some embodiments, a provided compound or pharmaceutically acceptable salt thereof, is selected from those wherein BBM is

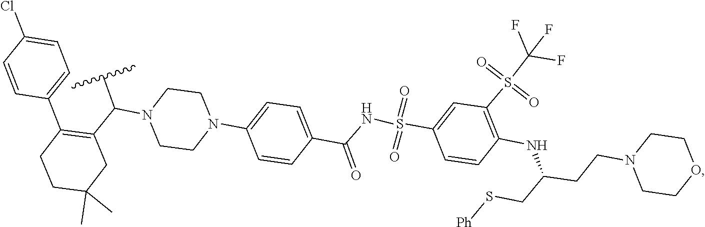 [SMILES: *C(C1=C(c2ccc(Cl)cc2)CCC(C)(C)C1)N1CCN(c2ccc(C(=O)NS(=O)(=O)c3ccc(N[C@H](CCN4CCOCC4)CSc4ccccc4)c(S(=O)(=O)C(F)(F)F)c3)cc2)CC1]

LBM is selected from any of those in Table A below, and L is selected from any of those in Table B below.

In some embodiments, a provided compound or pharmaceutically acceptable salt thereof, is selected from those wherein BBM is

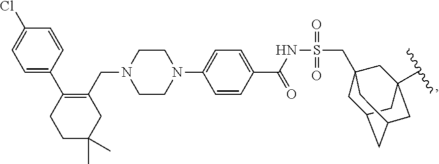 [SMILES: *C12CC3CC(C1)CC(CS(=O)(=O)NC(=O)c1ccc(N4CCN(CC5=C(c6ccc(Cl)cc6)CCC(C)(C)C5)CC4)cc1)(C3)C2]

LBM is selected from any of those in Table A below, and L is selected from any of those in Table B below.

In some embodiments, a provided compound or pharmaceutically acceptable salt thereof, is selected from those wherein BBM is

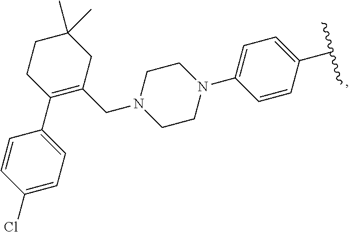 [SMILES: *c1ccc(N2CCN(CC3=C(c4ccc(Cl)cc4)CCC(C)(C)C3)CC2)cc1]

LBM is selected from any of those in Table A below, and L is selected from any of those in Table B below.

In some embodiments, a provided compound or pharmaceutically acceptable salt thereof, is selected from those wherein BBM is

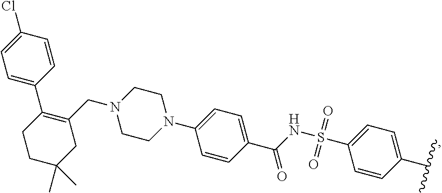 [SMILES: *c1ccc(S(=O)(=O)NC(=O)c2ccc(N3CCN(CC4=C(c5ccc(Cl)cc5)CCC(C)(C)C4)CC3)cc2)cc1]

LBM is selected from any of those in Table A below, and L is selected from any of those in Table B below.

In some embodiments, a provided compound or pharmaceutically acceptable salt thereof, is selected from those wherein BBM is

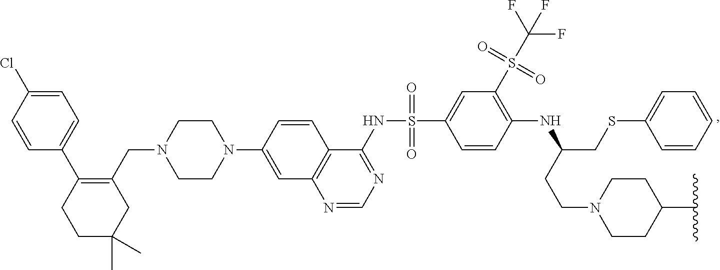 [SMILES: *C1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

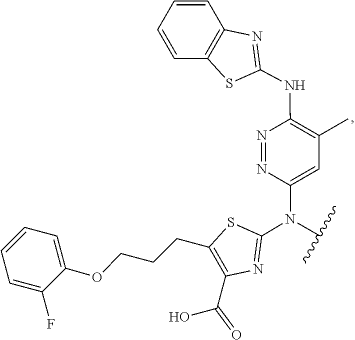 [SMILES: *N(c1cc(C)c(Nc2nc3ccccc3s2)nn1)c1nc(C(=O)O)c(CCCOc2ccccc2F)s1]

LBM is selected from any of those in Table A below, and L is selected from any of those in Table B below.

In some embodiments, a provided compound or pharmaceutically acceptable salt thereof, is selected from those wherein BBM is

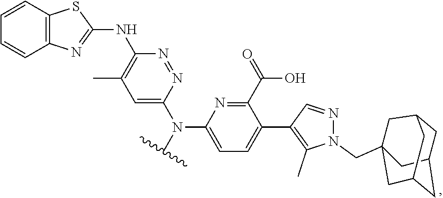 [SMILES: *N(c1cc(C)c(Nc2nc3ccccc3s2)nn1)c1ccc(-c2cnn(CC34CC5CC(CC(C5)C3)C4)c2C)c(C(=O)O)n1]

LBM is selected from any of those in Table A below, and L is selected from any of those in Table B below.

In some embodiments, a provided compound or pharmaceutically acceptable salt thereof, is selected from those wherein BBM is

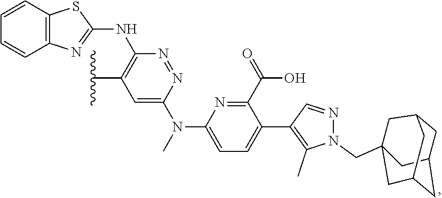 [SMILES: *c1cc(N(C)c2ccc(-c3cnn(CC45CC6CC(CC(C6)C4)C5)c3C)c(C(=O)O)n2)nnc1Nc1nc2ccccc2s1]

LBM is selected from any of those in Table A below, and L is selected from any of those in Table B below.

In some embodiments, a provided compound or pharmaceutically acceptable salt thereof, is selected from those wherein BBM is

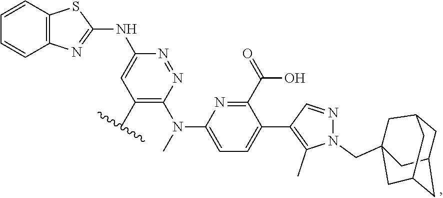 [SMILES: *c1cc(Nc2nc3ccccc3s2)nnc1N(C)c1ccc(-c2cnn(CC34CC5CC(CC(C5)C3)C4)c2C)c(C(=O)O)n1]

LBM is selected from any of those in Table A below, and L is selected from any of those in Table B below.

In some embodiments, a provided compound or pharmaceutically acceptable salt thereof, is selected from those wherein BBM is

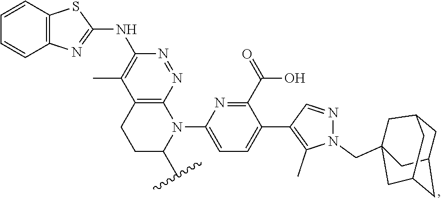 [SMILES: *C1CCc2c(nnc(Nc3nc4ccccc4s3)c2C)N1c1ccc(-c2cnn(CC34CC5CC(CC(C5)C3)C4)c2C)c(C(=O)O)n1]

LBM is selected from any of those in Table A below, and L is selected from any of those in Table B below.

In some embodiments, a provided compound or pharmaceutically acceptable salt thereof, is selected from those wherein BBM is

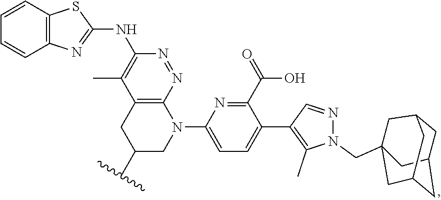 [SMILES: *C1Cc2c(nnc(Nc3nc4ccccc4s3)c2C)N(c2ccc(-c3cnn(CC45CC6CC(CC(C6)C4)C5)c3C)c(C(=O)O)n2)C1]

LBM is selected from any of those in Table A below, and L is selected from any of those in Table B below.

In some embodiments, a provided compound or pharmaceutically acceptable salt thereof, is selected from those wherein BBM is

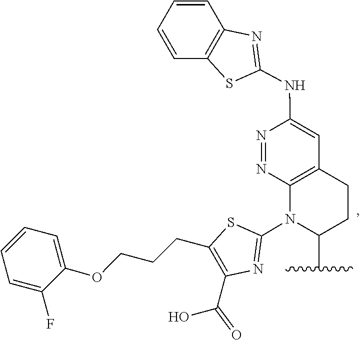 [SMILES: *C1CCc2cc(Nc3nc4ccccc4s3)nnc2N1c1nc(C(=O)O)c(CCCOc2ccccc2F)s1]

LBM is selected from any of those in Table A below, and L is selected from any of those in Table B below.

In some embodiments, a provided compound or pharmaceutically acceptable salt thereof, is selected from those wherein BBM is

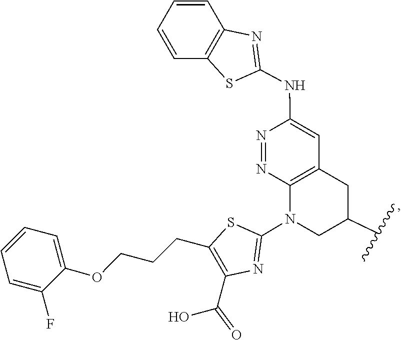 [SMILES: *C1Cc2cc(Nc3nc4ccccc4s3)nnc2N(c2nc(C(=O)O)c(CCCOc3ccccc3F)s2)C1]

LBM is selected from any of those in Table A below, and L is selected from any of those in Table B below.

In some embodiments, a provided compound or pharmaceutically acceptable salt thereof, is selected from those wherein BBM is

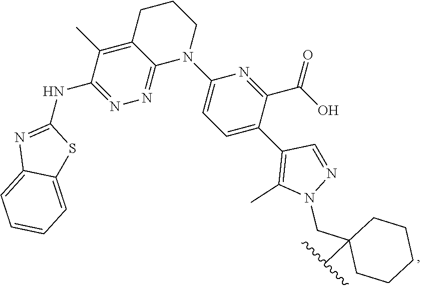 [SMILES: *C1(Cn2ncc(-c3ccc(N4CCCc5c4nnc(Nc4nc6ccccc6s4)c5C)nc3C(=O)O)c2C)CCCCC1]

LBM is selected from any of those in Table A below, and L is selected from any of those in Table B below.

In some embodiments, a provided compound or pharmaceutically acceptable salt thereof, is selected from those wherein BBM is

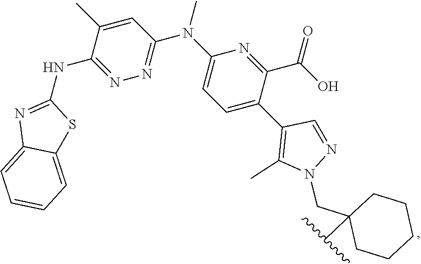 [SMILES: *C1(Cn2ncc(-c3ccc(N(C)c4cc(C)c(Nc5nc6ccccc6s5)nn4)nc3C(=O)O)c2C)CCCCC1]

LBM is selected from any of those in Table A below, and L is selected from any of those in Table B below.

In some embodiments, a provided compound or pharmaceutically acceptable salt thereof, is selected from those wherein BBM is

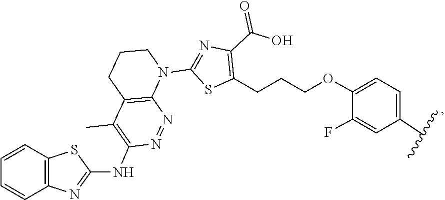 [SMILES: *c1ccc(OCCCc2sc(N3CCCc4c3nnc(Nc3nc5ccccc5s3)c4C)nc2C(=O)O)c(F)c1]

LBM is selected from any of those in Table A below, and L is selected from any of those in Table B below.

In some embodiments, a provided compound or pharmaceutically acceptable salt thereof, is selected from those wherein BBM is

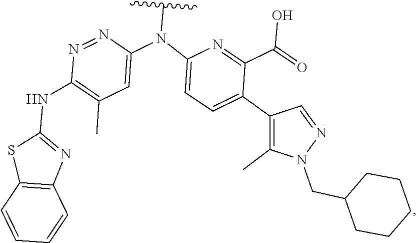 [SMILES: *N(c1cc(C)c(Nc2nc3ccccc3s2)nn1)c1ccc(-c2cnn(CC3CCCCC3)c2C)c(C(=O)O)n1]

LBM is selected from any of those in Table A below, and L is selected from any of those in Table B below.

In some embodiments, a provided compound or pharmaceutically acceptable salt thereof, is selected from those wherein BBM is

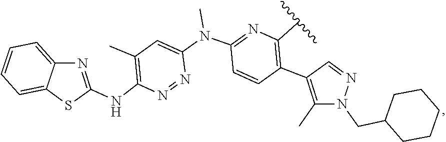 [SMILES: *c1nc(N(C)c2cc(C)c(Nc3nc4ccccc4s3)nn2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

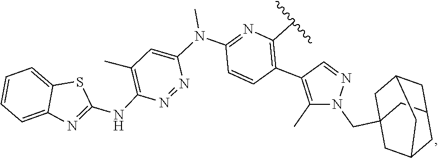 [SMILES: *c1nc(N(C)c2cc(C)c(Nc3nc4ccccc4s3)nn2)ccc1-c1cnn(CC23CC4CC(CC(C4)C2)C3)c1C]

LBM is selected from any of those in Table A below, and L is selected from any of those in Table B below.

In some embodiments, a provided compound or pharmaceutically acceptable salt thereof, is selected from those wherein BBM is

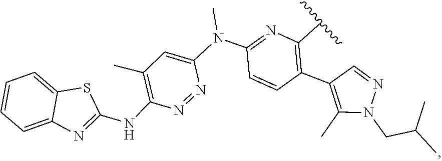 [SMILES: *c1nc(N(C)c2cc(C)c(Nc3nc4ccccc4s3)nn2)ccc1-c1cnn(CC(C)C)c1C]

LBM is selected from any of those in Table A below, and L is selected from any of those in Table B below.

In some embodiments, a provided compound or pharmaceutically acceptable salt thereof, is selected from those wherein BBM is

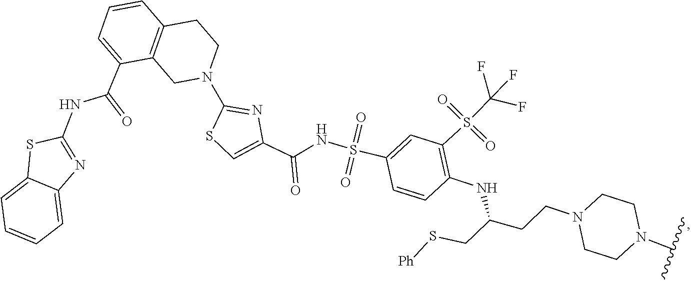 [SMILES: *N1CCN(CC[C@H](CSc2ccccc2)Nc2ccc(S(=O)(=O)NC(=O)c3csc(N4CCc5cccc(C(=O)Nc6nc7ccccc7s6)c5C4)n3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

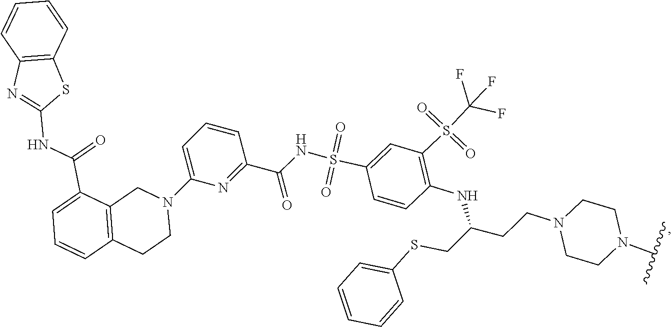 [SMILES: *N1CCN(CC[C@H](CSc2ccccc2)Nc2ccc(S(=O)(=O)NC(=O)c3cccc(N4CCc5cccc(C(=O)Nc6nc7ccccc7s6)c5C4)n3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

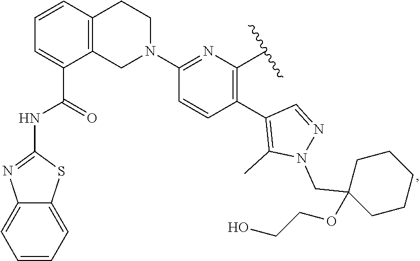 [SMILES: *c1nc(N2CCc3cccc(C(=O)Nc4nc5ccccc5s4)c3C2)ccc1-c1cnn(CC2(OCCO)CCCCC2)c1C]

LBM is selected from any of those in Table A below, and L is selected from any of those in Table B below.

In some embodiments, a provided compound or pharmaceutically acceptable salt thereof, is selected from those wherein BBM is

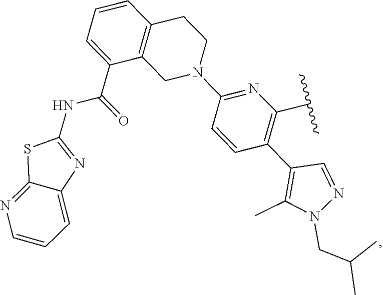 [SMILES: *c1nc(N2CCc3cccc(C(=O)Nc4nc5cccnc5s4)c3C2)ccc1-c1cnn(CC(C)C)c1C]

LBM is selected from any of those in Table A below, and L is selected from any of those in Table B below.

In some embodiments, a provided compound or pharmaceutically acceptable salt thereof, is selected from those wherein BBM is

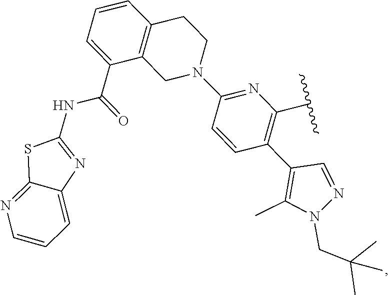 [SMILES: *c1nc(N2CCc3cccc(C(=O)Nc4nc5cccnc5s4)c3C2)ccc1-c1cnn(CC(C)(C)C)c1C]

LBM is selected from any of those in Table A below, and L is selected from any of those in Table B below.

In some embodiments, a provided compound or pharmaceutically acceptable salt thereof, is selected from those wherein BBM is

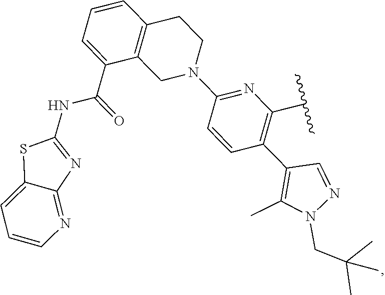 [SMILES: *c1nc(N2CCc3cccc(C(=O)Nc4nc5ncccc5s4)c3C2)ccc1-c1cnn(CC(C)(C)C)c1C]

LBM is selected from any of those in Table A below, and L is selected from any of those in Table B below.

In some embodiments, a provided compound or pharmaceutically acceptable salt thereof, is selected from those wherein BBM is

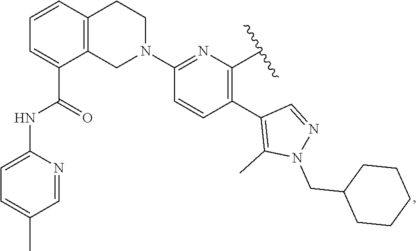 [SMILES: *c1nc(N2CCc3cccc(C(=O)Nc4ccc(C)cn4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

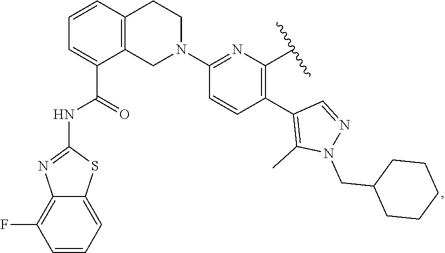 [SMILES: *c1nc(N2CCc3cccc(C(=O)Nc4nc5c(F)cccc5s4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

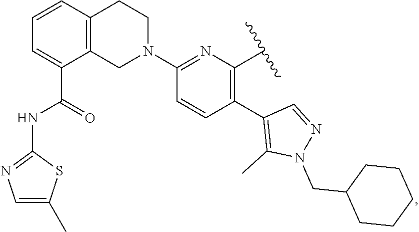 [SMILES: *c1nc(N2CCc3cccc(C(=O)Nc4ncc(C)s4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

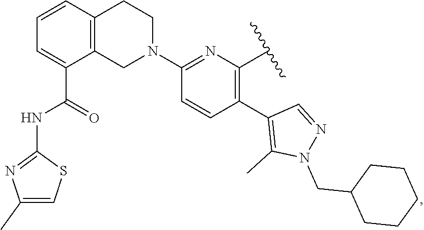 [SMILES: *c1nc(N2CCc3cccc(C(=O)Nc4nc(C)cs4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

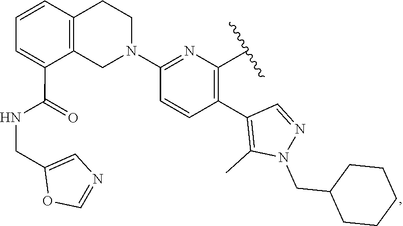 [SMILES: *c1nc(N2CCc3cccc(C(=O)NCc4cnco4)c3C2)ccc1-c1cnn(CC2CCCCC2)c1C]

LBM is selected from any of those in Table A below, and L is selected from any of those in Table B below.

In some embodiments, a provided compound or pharmaceutically acceptable salt thereof, is selected from those wherein BBM is

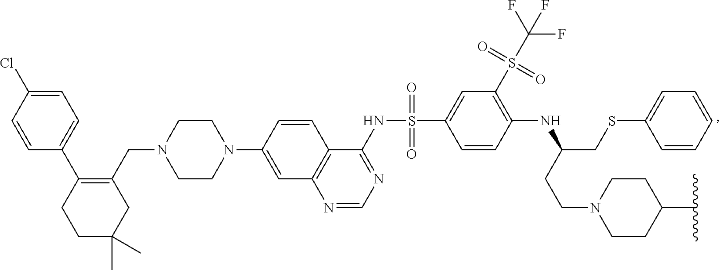 [SMILES: *C1CCN(CC[C@H](CSc2ccccc2)Nc2ccc(S(=O)(=O)Nc3ncnc4cc(N5CCN(CC6=C(c7ccc(Cl)cc7)CCC(C)(C)C6)CC5)ccc34)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

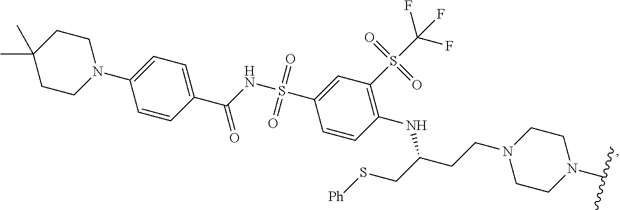 [SMILES: *N1CCN(CC[C@H](CSc2ccccc2)Nc2ccc(S(=O)(=O)NC(=O)c3ccc(N4CCC(C)(C)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

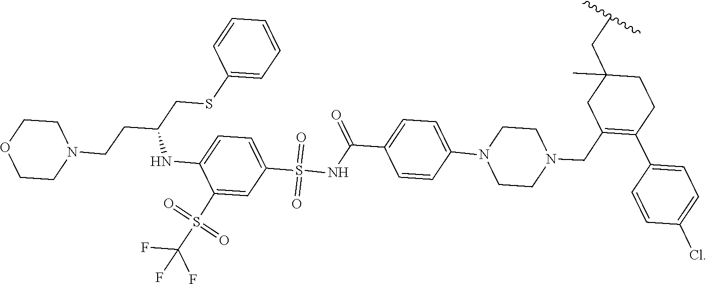 [SMILES: *CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

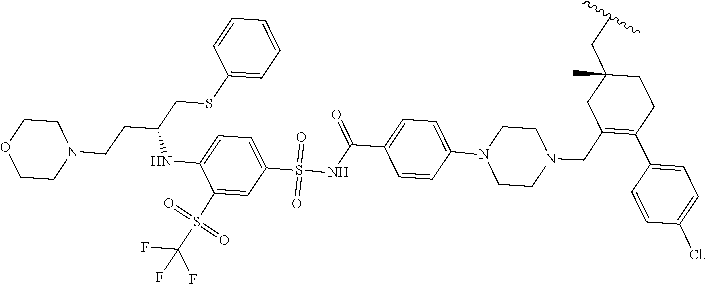 [SMILES: *C[C@]1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

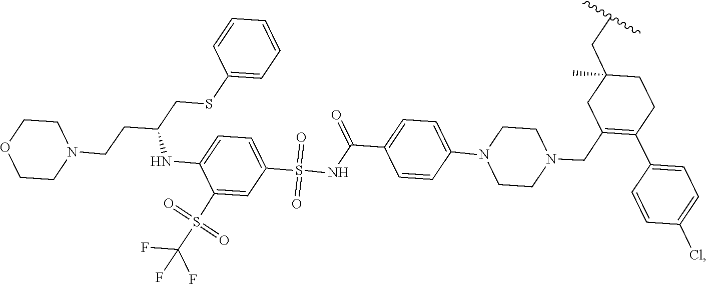 [SMILES: *C[C@@]1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

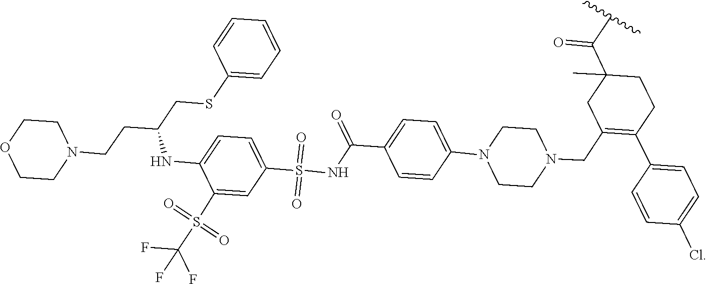 [SMILES: *C(=O)C1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

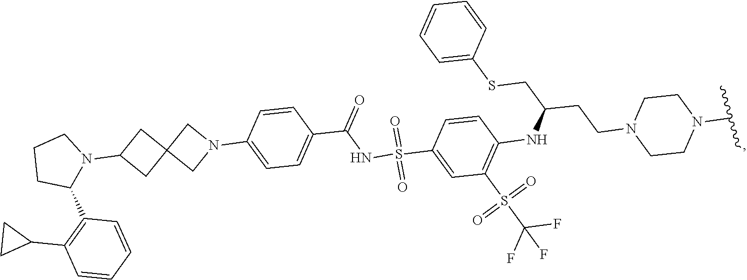 [SMILES: *N1CCN(CC[C@H](CSc2ccccc2)Nc2ccc(S(=O)(=O)NC(=O)c3ccc(N4CC5(CC(N6CCC[C@H]6c6ccccc6C6CC6)C5)C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

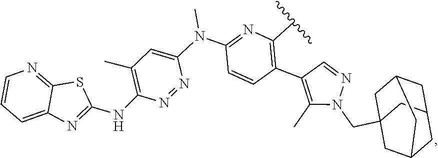 [SMILES: *c1nc(N(C)c2cc(C)c(Nc3nc4cccnc4s3)nn2)ccc1-c1cnn(CC23CC4CC(CC(C4)C2)C3)c1C]

LBM is selected from any of those in Table A below, and L is selected from any of those in Table B below.

In some embodiments, a provided compound or pharmaceutically acceptable salt thereof, is selected from those wherein BBM is

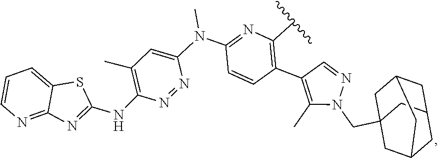 [SMILES: *c1nc(N(C)c2cc(C)c(Nc3nc4ncccc4s3)nn2)ccc1-c1cnn(CC23CC4CC(CC(C4)C2)C3)c1C]

LBM is selected from any of those in Table A below, and L is selected from any of those in Table B below.

In some embodiments, a provided compound or pharmaceutically acceptable salt thereof, is selected from those wherein BBM is

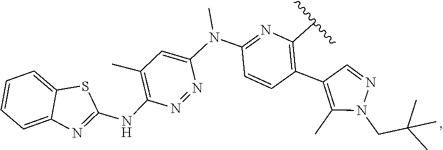 [SMILES: *c1nc(N(C)c2cc(C)c(Nc3nc4ccccc4s3)nn2)ccc1-c1cnn(CC(C)(C)C)c1C]

LBM is selected from any of those in Table A below, and L is selected from any of those in Table B below.

In some embodiments, a provided compound or pharmaceutically acceptable salt thereof, is selected from those wherein BBM is

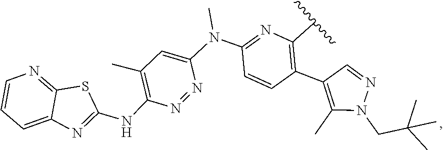 [SMILES: *c1nc(N(C)c2cc(C)c(Nc3nc4cccnc4s3)nn2)ccc1-c1cnn(CC(C)(C)C)c1C]

LBM is selected from any of those in Table A below, and L is selected from any of those in Table B below.

In some embodiments, a provided compound or pharmaceutically acceptable salt thereof, is selected from those wherein BBM is

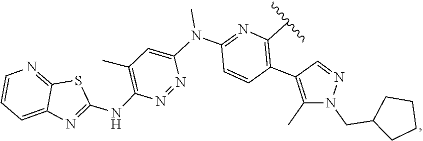 [SMILES: *c1nc(N(C)c2cc(C)c(Nc3nc4cccnc4s3)nn2)ccc1-c1cnn(CC2CCCC2)c1C]

LBM is selected from any of those in Table A below, and L is selected from any of those in Table B below.

TABLE A
Exemplified E3 Ligase Binding Moiety (LBM)

| | |
|---|---|
| 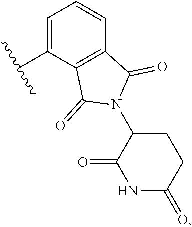 [SMILES: *c1cccc2c1C(=O)N(C1CCC(=O)NC1=O)C2=O] | (a) |
| 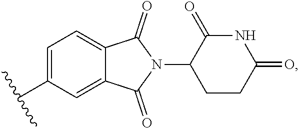 [SMILES: *c1ccc2c(c1)C(=O)N(C1CCC(=O)NC1=O)C2=O] | (b) |
| 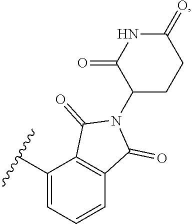 [SMILES: *c1cccc2c1C(=O)N(C1CCC(=O)NC1=O)C2=O] | (c) |
| 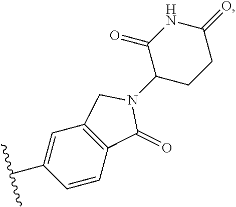 [SMILES: *c1ccc2c(c1)CN(C1CCC(=O)NC1=O)C2=O] | (d) |
| 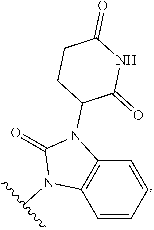 [SMILES: *n1c(=O)n(C2CCC(=O)NC2=O)c2ccccc21 ; ","] | (e) |
| 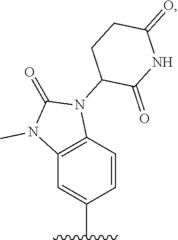 [SMILES: *c1ccc2c(c1)n(C)c(=O)n2C1CCC(=O)NC1=O] | (f) |
| 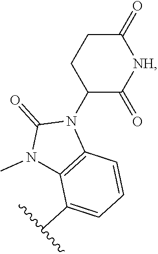 [SMILES: *c1cccc2c1n(C)c(=O)n2C1CCC(=O)NC1=O] | (g) |
| 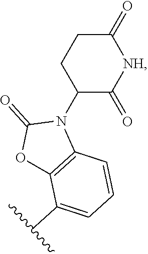 [SMILES: *c1cccc2c1oc(=O)n2C1CCC(=O)NC1=O] | (h) |
| 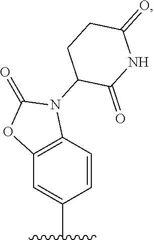 [SMILES: *c1ccc2c(c1)oc(=O)n2C1CCC(=O)NC1=O] | (i) |
| 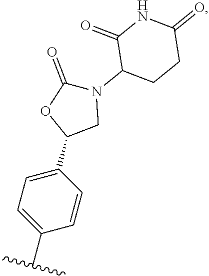 [SMILES: *c1ccc([C@H]2CN(C3CCC(=O)NC3=O)C(=O)O2)cc1] | (j) |
| 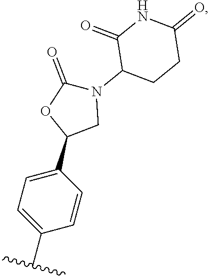 [SMILES: *c1ccc([C@@H]2CN(C3CCC(=O)NC3=O)C(=O)O2)cc1] | (k) |
| 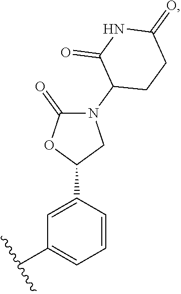 [SMILES: *c1cccc([C@H]2CN(C3CCC(=O)NC3=O)C(=O)O2)c1] | (l) |
| 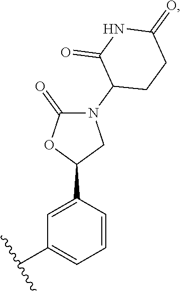 [SMILES: *c1cccc([C@@H]2CN(C3CCC(=O)NC3=O)C(=O)O2)c1] | (m) |
| 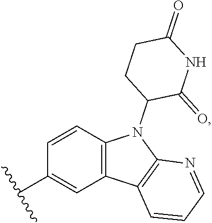 [SMILES: *c1ccc2c(c1)c1cccnc1n2C1CCC(=O)NC1=O] | (n) |
| 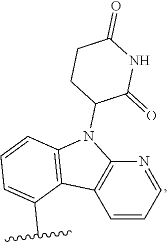 [SMILES: *c1cccc2c1c1cccnc1n2C1CCC(=O)NC1=O ; ","] | (o) |
| 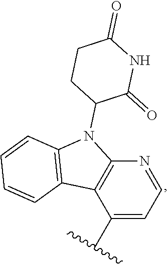 [SMILES: *c1ccnc2c1c1ccccc1n2C1CCC(=O)NC1=O ; ","] | (p) |
| 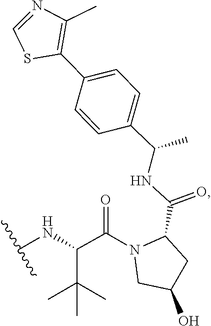 [SMILES: *N[C@H](C(=O)N1C[C@H](O)C[C@H]1C(=O)N[C@@H](C)c1ccc(-c2scnc2C)cc1)C(C)(C)C] | (q) |
| 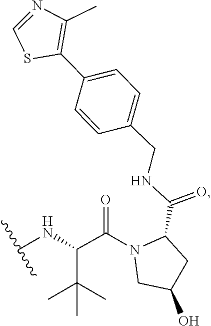 [SMILES: *N[C@H](C(=O)N1C[C@H](O)C[C@H]1C(=O)NCc1ccc(-c2scnc2C)cc1)C(C)(C)C] | (r) |
| 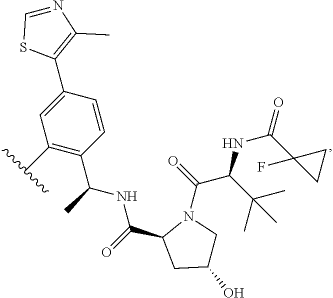 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C ; ","] | (s) |
| 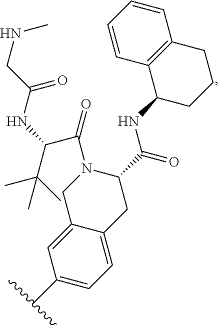 [SMILES: *c1ccc2c(c1)CN(C(=O)[C@@H](NC(=O)CNC)C(C)(C)C)[C@H](C(=O)N[C@@H]1CCCc3ccccc31)C2 ; ","] | (t) |
| 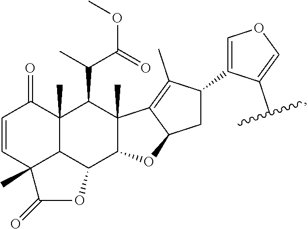 | (u) |
| 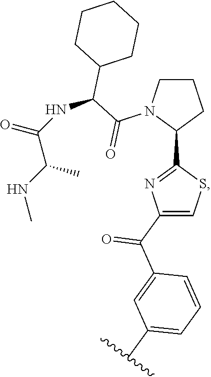 [SMILES: *c1cccc(C(=O)c2csc([C@@H]3CCCN3C(=O)[C@@H](NC(=O)[C@H](C)NC)C3CCCCC3)n2)c1] | (v) |
| 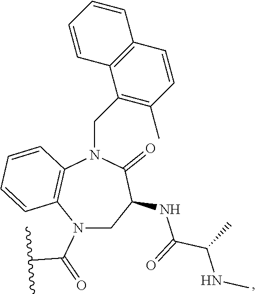 [SMILES: *C(=O)N1C[C@H](NC(=O)[C@H](C)NC)C(=O)N(Cc2c(C)ccc3ccccc23)c2ccccc21 ; ","] | (w) |
| 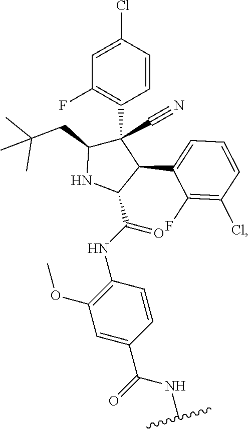 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (x) |
| 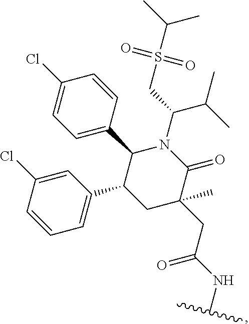 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O ; ","] | (y) |
| 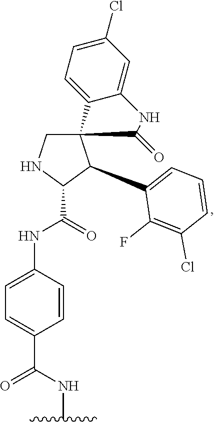 [SMILES: *NC(=O)c1ccc(NC(=O)[C@@H]2NC[C@@]3(C(=O)Nc4cc(Cl)ccc43)[C@H]2c2cccc(Cl)c2F)cc1 ; ","] | (z) |
| 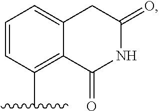 [SMILES: *c1cccc2c1C(=O)NC(=O)C2] | (aa) |
| 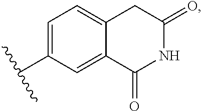 [SMILES: *c1ccc2c(c1)C(=O)NC(=O)C2] | (bb) |
| 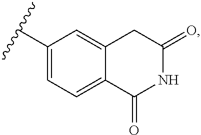 [SMILES: *c1ccc2c(c1)CC(=O)NC2=O] | (cc) |
| 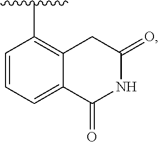 [SMILES: *c1cccc2c1CC(=O)NC2=O] | (dd) |
| 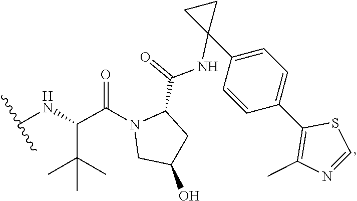 [SMILES: *N[C@H](C(=O)N1C[C@H](O)C[C@H]1C(=O)NC1(c2ccc(-c3scnc3C)cc2)CC1)C(C)(C)C ; ","] | (ee) |
| 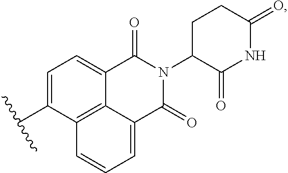 [SMILES: *c1ccc2c3c(cccc13)C(=O)N(C1CCC(=O)NC1=O)C2=O] | (ff) |
| 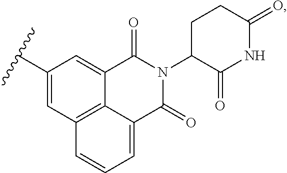 [SMILES: *c1cc2c3c(cccc3c1)C(=O)N(C1CCC(=O)NC1=O)C2=O] | (gg) |
| 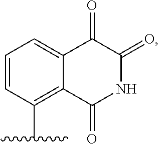 [SMILES: *c1cccc2c1C(=O)NC(=O)C2=O] | (hh) |
| 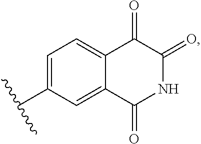 [SMILES: *c1ccc2c(c1)C(=O)NC(=O)C2=O] | (ii) |
| 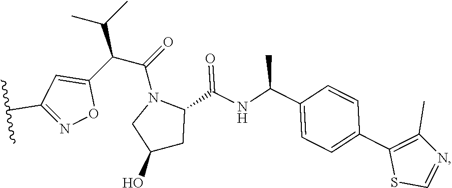 [SMILES: *c1cc([C@H](C(=O)N2C[C@H](O)C[C@H]2C(=O)N[C@@H](C)c2ccc(-c3scnc3C)cc2)C(C)C)on1] | (jj) |
| 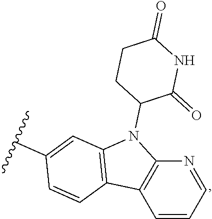 [SMILES: *c1ccc2c3cccnc3n(C3CCC(=O)NC3=O)c2c1 ; ","] | (kk) |
| 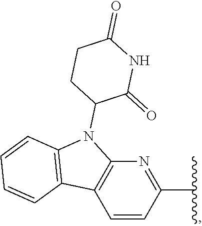 [SMILES: *c1ccc2c3ccccc3n(C3CCC(=O)NC3=O)c2n1] | (ll) |
| 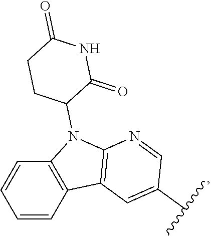 [SMILES: *c1cnc2c(c1)c1ccccc1n2C1CCC(=O)NC1=O ; ","] | (mm) |
| 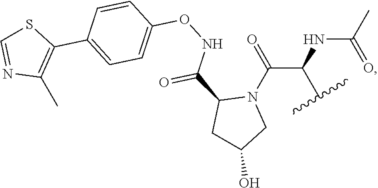 [SMILES: *[C@H](NC(C)=O)C(=O)N1C[C@H](O)C[C@H]1C(=O)NOc1ccc(-c2scnc2C)cc1] | (nn) |
| 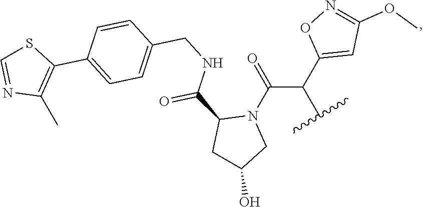 [SMILES: *C(C(=O)N1C[C@H](O)C[C@H]1C(=O)NCc1ccc(-c2scnc2C)cc1)c1cc(OC)no1 ; ","] | (oo) |
| 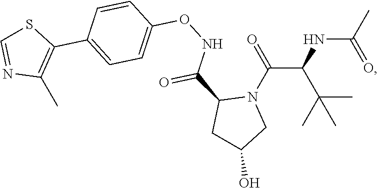 [SMILES: CC(=O)N[C@H](C(=O)N1C[C@H](O)C[C@H]1C(=O)NOc1ccc(-c2scnc2C)cc1)C(C)(C)C] | (pp) |
| 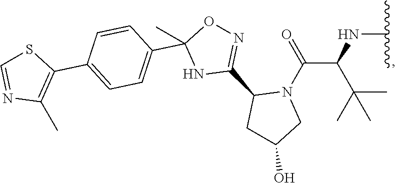 [SMILES: *N[C@H](C(=O)N1C[C@H](O)C[C@H]1C1=NOC(C)(c2ccc(-c3scnc3C)cc2)N1)C(C)(C)C] | (qq) |
| 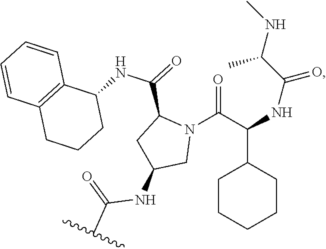 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1] | (rr) |
| 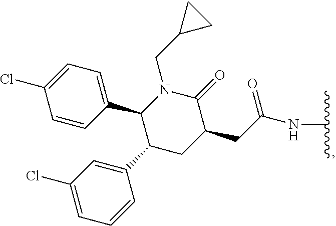 [SMILES: *NC(=O)C[C@H]1C[C@H](c2cccc(Cl)c2)[C@@H](c2ccc(Cl)cc2)N(CC2CC2)C1=O] | (ss) |
| 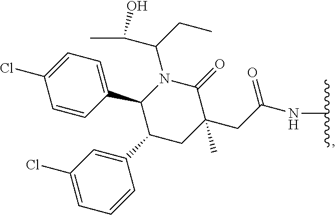 [SMILES: *NC(=O)C[C@@]1(C)C[C@H](c2cccc(Cl)c2)[C@@H](c2ccc(Cl)cc2)N(C(CC)[C@H](C)O)C1=O] | (tt) |
| 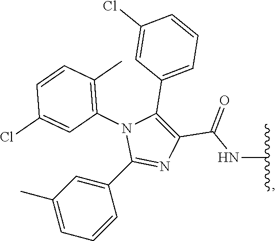 [SMILES: *NC(=O)c1nc(-c2cccc(C)c2)n(-c2cc(Cl)ccc2C)c1-c1cccc(Cl)c1] | (uu) |
| 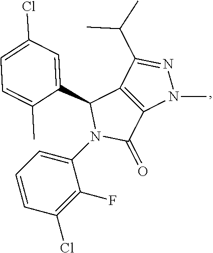 [SMILES: Cc1ccc(Cl)cc1[C@H]1c2c(C(C)C)nn(C)c2C(=O)N1c1cccc(Cl)c1F ; ","] | (vv) |
| 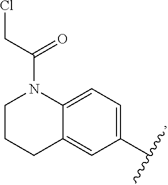 | (ww) |
| 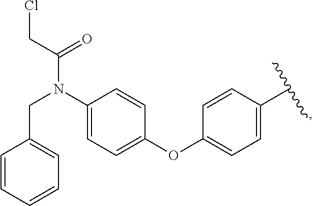 [SMILES: *c1ccc(Oc2ccc(N(Cc3ccccc3)C(=O)CCl)cc2)cc1 ; ","] | (xx) |
| 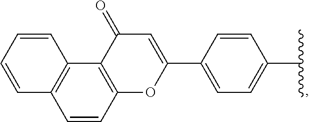 [SMILES: *c1ccc(-c2cc(=O)c3c(ccc4ccccc43)o2)cc1] | (vv) |
| 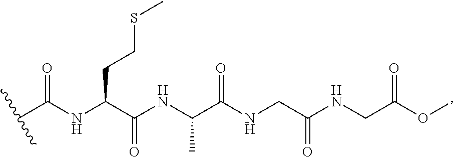 | (zz) |
| 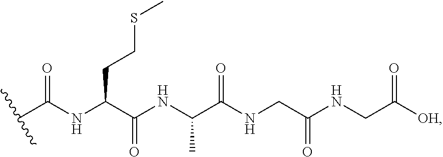 [SMILES: *C(=O)N[C@@H](CCSC)C(=O)N[C@@H](C)C(=O)NCC(=O)NCC(=O)O] | (aaa) |
| 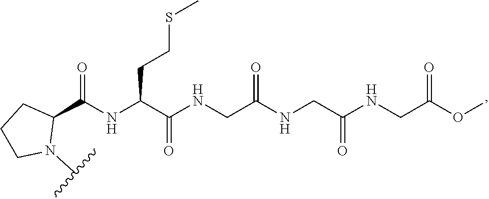 | (bbb) |
| 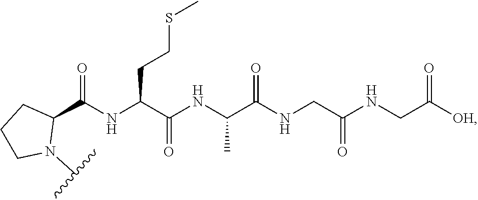 [SMILES: *N1CCC[C@H]1C(=O)N[C@@H](CCSC)C(=O)N[C@@H](C)C(=O)NCC(=O)NCC(=O)O] | (ccc) |
| 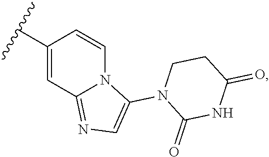 [SMILES: *c1ccn2c(N3CCC(=O)NC3=O)cnc2c1] | (ddd) |
| 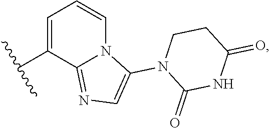 [SMILES: *c1cccn2c(N3CCC(=O)NC3=O)cnc12] | (eee) |
| 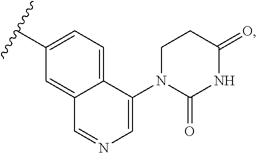 [SMILES: *c1ccc2c(N3CCC(=O)NC3=O)cncc2c1] | (fff) |
| 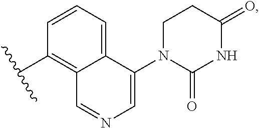 [SMILES: *c1cccc2c(N3CCC(=O)NC3=O)cncc12] | (ggg) |
| 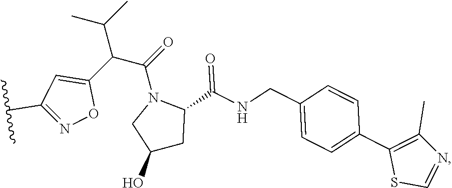 [SMILES: *c1cc(C(C(=O)N2C[C@H](O)C[C@H]2C(=O)NCc2ccc(-c3scnc3C)cc2)C(C)C)on1] | (hhh) |
| 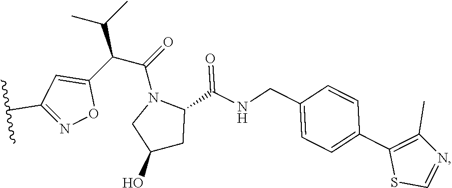 [SMILES: *c1cc([C@H](C(=O)N2C[C@H](O)C[C@H]2C(=O)NCc2ccc(-c3scnc3C)cc2)C(C)C)on1] | (iii) |
| 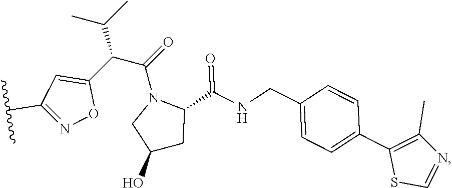 [SMILES: *c1cc([C@@H](C(=O)N2C[C@H](O)C[C@H]2C(=O)NCc2ccc(-c3scnc3C)cc2)C(C)C)on1] | (jjj) |
| 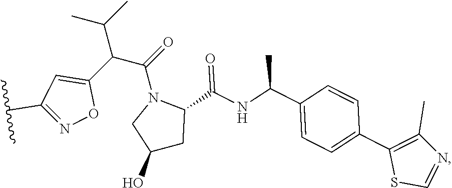 [SMILES: *c1cc(C(C(=O)N2C[C@H](O)C[C@H]2C(=O)N[C@@H](C)c2ccc(-c3scnc3C)cc2)C(C)C)on1] | (kkk) |
| 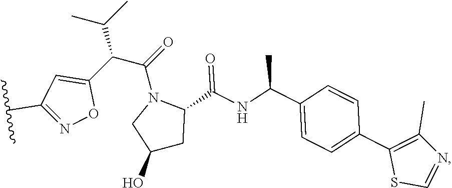 [SMILES: *c1cc([C@@H](C(=O)N2C[C@H](O)C[C@H]2C(=O)N[C@@H](C)c2ccc(-c3scnc3C)cc2)C(C)C)on1] | (lll) |
| 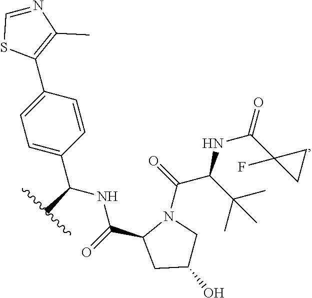 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1 ; ","] | (mmm) |
| 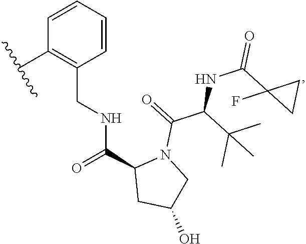 [SMILES: *c1ccccc1CNC(=O)[C@@H]1C[C@@H](O)CN1C(=O)[C@@H](NC(=O)C1(F)CC1)C(C)(C)C ; ","] | (nnn) |
| 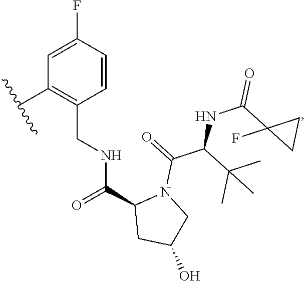 [SMILES: *c1cc(F)ccc1CNC(=O)[C@@H]1C[C@@H](O)CN1C(=O)[C@@H](NC(=O)C1(F)CC1)C(C)(C)C ; ","] | (ooo) |
| 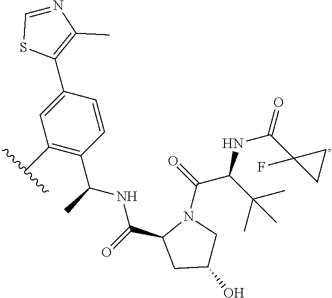 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C ; ","] | (ppp) |
| 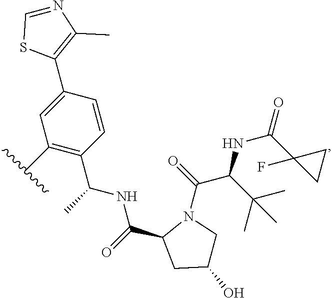 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C ; ","] | (qqq) |
| 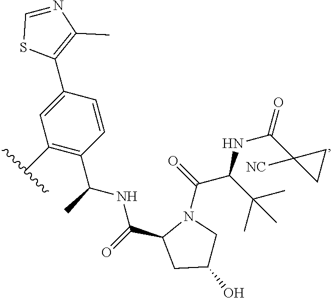 [SMILES: *c1cc(-c2scnc2C)ccc1[C@H](C)NC(=O)[C@@H]1C[C@@H](O)CN1C(=O)[C@@H](NC(=O)C1(C#N)CC1)C(C)(C)C ; ","] | (rrr) |
| 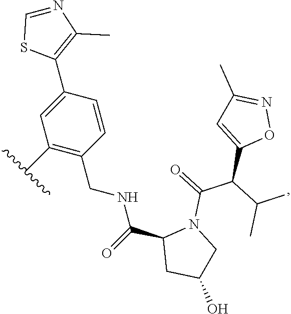 | (sss) |
| 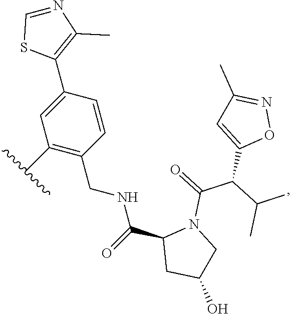 | (ttt) |
| 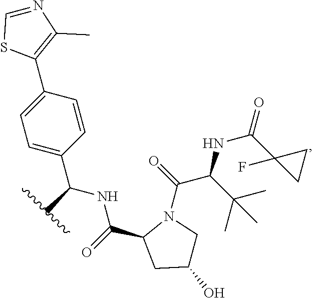 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1 ; ","] | (uuu) |
| 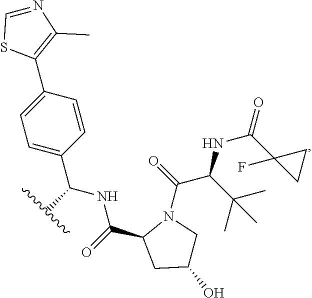 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1 ; ","] | (vvv) |
| 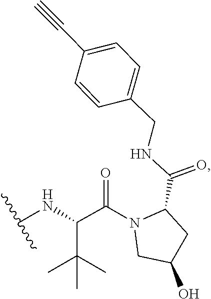 [SMILES: *N[C@H](C(=O)N1C[C@H](O)C[C@H]1C(=O)NCc1ccc(C#C)cc1)C(C)(C)C] | (www) |
| 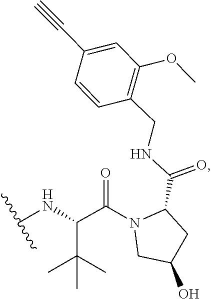 [SMILES: *N[C@H](C(=O)N1C[C@H](O)C[C@H]1C(=O)NCc1ccc(C#C)cc1OC)C(C)(C)C] | (xxx) |
| 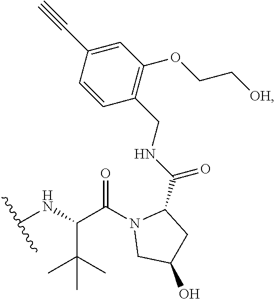 [SMILES: *N[C@H](C(=O)N1C[C@H](O)C[C@H]1C(=O)NCc1ccc(C#C)cc1OCCO)C(C)(C)C] | (yyy) |
| 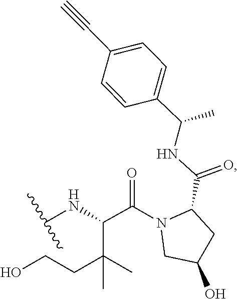 [SMILES: *N[C@H](C(=O)N1C[C@H](O)C[C@H]1C(=O)N[C@@H](C)c1ccc(C#C)cc1)C(C)(C)CCO] | (zzz) |
| 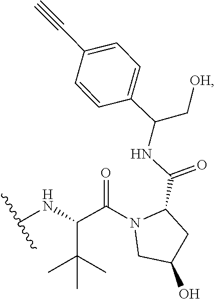 [SMILES: *N[C@H](C(=O)N1C[C@H](O)C[C@H]1C(=O)NC(CO)c1ccc(C#C)cc1)C(C)(C)C] | (aaaa) |
| 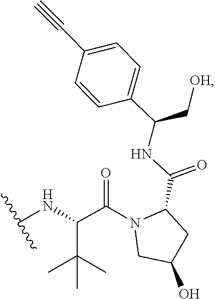 [SMILES: *N[C@H](C(=O)N1C[C@H](O)C[C@H]1C(=O)N[C@H](CO)c1ccc(C#C)cc1)C(C)(C)C] | (bbbb) |
| 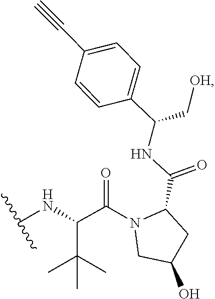 [SMILES: *N[C@H](C(=O)N1C[C@H](O)C[C@H]1C(=O)N[C@@H](CO)c1ccc(C#C)cc1)C(C)(C)C] | (cccc) |
| 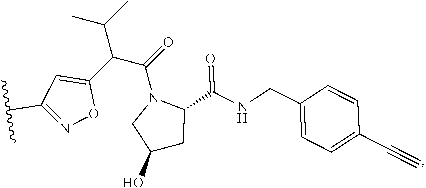 [SMILES: *c1cc(C(C(=O)N2C[C@H](O)C[C@H]2C(=O)NCc2ccc(C#C)cc2)C(C)C)on1 ; ","] | (dddd) |
| 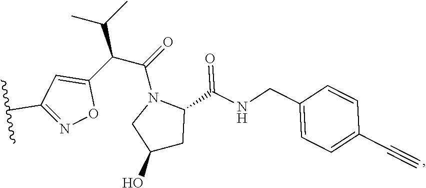 [SMILES: *c1cc([C@H](C(=O)N2C[C@H](O)C[C@H]2C(=O)NCc2ccc(C#C)cc2)C(C)C)on1 ; ","] | (eeee) |
| 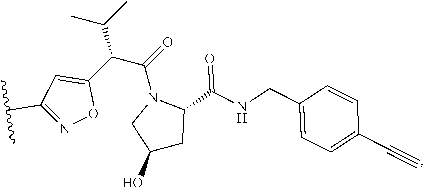 [SMILES: *c1cc([C@@H](C(=O)N2C[C@H](O)C[C@H]2C(=O)NCc2ccc(C#C)cc2)C(C)C)on1 ; ","] | (ffff) |
| 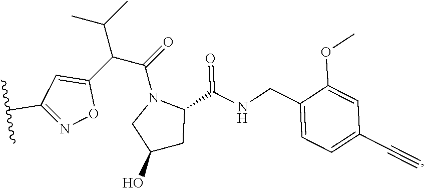 [SMILES: *c1cc(C(C(=O)N2C[C@H](O)C[C@H]2C(=O)NCc2ccc(C#C)cc2OC)C(C)C)on1 ; ","] | (gggg) |
| 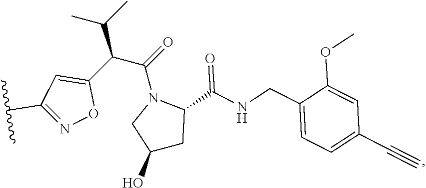 [SMILES: *c1cc([C@H](C(=O)N2C[C@H](O)C[C@H]2C(=O)NCc2ccc(C#C)cc2OC)C(C)C)on1 ; ","] | (hhhh) |
| 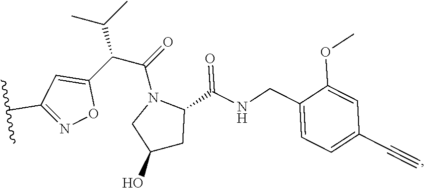 [SMILES: *c1cc([C@@H](C(=O)N2C[C@H](O)C[C@H]2C(=O)NCc2ccc(C#C)cc2OC)C(C)C)on1 ; ","] | (iiii) |
| 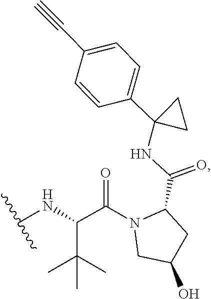 [SMILES: *N[C@H](C(=O)N1C[C@H](O)C[C@H]1C(=O)NC1(c2ccc(C#C)cc2)CC1)C(C)(C)C] | (jjjj) |
| 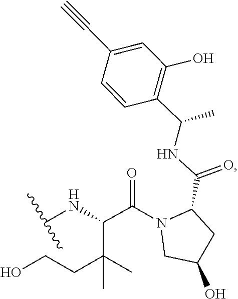 [SMILES: *N[C@H](C(=O)N1C[C@H](O)C[C@H]1C(=O)N[C@@H](C)c1ccc(C#C)cc1O)C(C)(C)CCO] | (kkkk) |
| 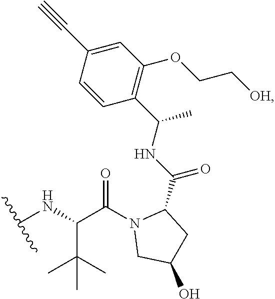 [SMILES: *N[C@H](C(=O)N1C[C@H](O)C[C@H]1C(=O)N[C@@H](C)c1ccc(C#C)cc1OCCO)C(C)(C)C] | (llll) |
| 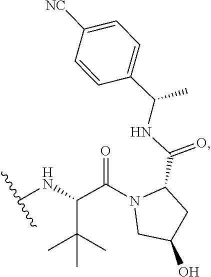 [SMILES: *N[C@H](C(=O)N1C[C@H](O)C[C@H]1C(=O)N[C@@H](C)c1ccc(C#N)cc1)C(C)(C)C] | (mmmm) |
| 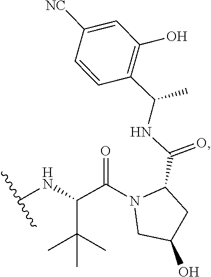 [SMILES: *N[C@H](C(=O)N1C[C@H](O)C[C@H]1C(=O)N[C@@H](C)c1ccc(C#N)cc1O)C(C)(C)C] | (nnnn) |
| 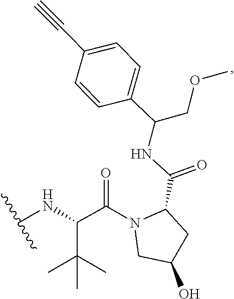 [SMILES: *N[C@H](C(=O)N1C[C@H](O)C[C@H]1C(=O)NC(COC)c1ccc(C#C)cc1)C(C)(C)C ; ","] | (oooo) |
| 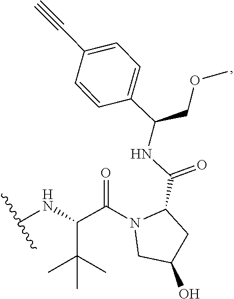 [SMILES: *N[C@H](C(=O)N1C[C@H](O)C[C@H]1C(=O)N[C@H](COC)c1ccc(C#C)cc1)C(C)(C)C ; ","] | (pppp) |
| 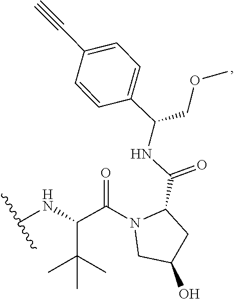 [SMILES: *N[C@H](C(=O)N1C[C@H](O)C[C@H]1C(=O)N[C@@H](COC)c1ccc(C#C)cc1)C(C)(C)C ; ","] | (qqqq) |
| 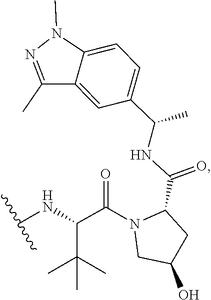 [SMILES: *N[C@H](C(=O)N1C[C@H](O)C[C@H]1C(=O)N[C@@H](C)c1ccc2c(c1)c(C)nn2C)C(C)(C)C] | (rrrr) |
| 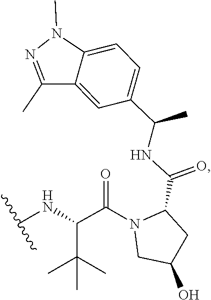 [SMILES: *N[C@H](C(=O)N1C[C@H](O)C[C@H]1C(=O)N[C@H](C)c1ccc2c(c1)c(C)nn2C)C(C)(C)C] | (ssss) |
| 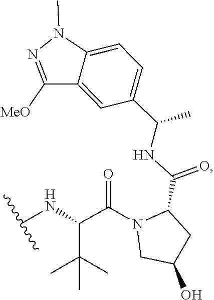 [SMILES: *N[C@H](C(=O)N1C[C@H](O)C[C@H]1C(=O)N[C@@H](C)c1ccc2c(c1)c(OC)nn2C)C(C)(C)C] | (tttt) |
| 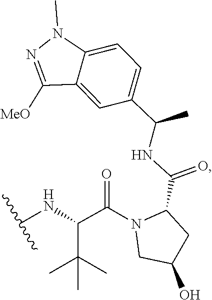 [SMILES: *N[C@H](C(=O)N1C[C@H](O)C[C@H]1C(=O)N[C@H](C)c1ccc2c(c1)c(OC)nn2C)C(C)(C)C] | (uuuu) |
| 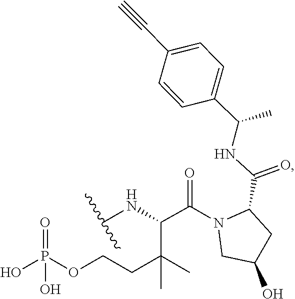 [SMILES: *N[C@H](C(=O)N1C[C@H](O)C[C@H]1C(=O)N[C@@H](C)c1ccc(C#C)cc1)C(C)(C)CCOP(=O)(O)O] | (vvvv) |
| 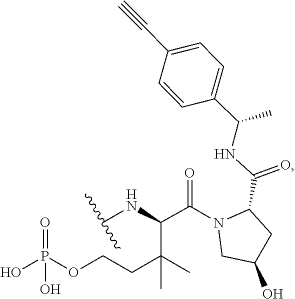 [SMILES: *N[C@@H](C(=O)N1C[C@H](O)C[C@H]1C(=O)N[C@@H](C)c1ccc(C#C)cc1)C(C)(C)CCOP(=O)(O)O] | (wwww) |
| 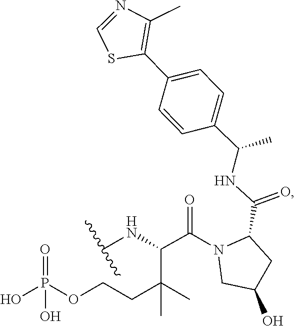 [SMILES: *N[C@H](C(=O)N1C[C@H](O)C[C@H]1C(=O)N[C@@H](C)c1ccc(-c2scnc2C)cc1)C(C)(C)CCOP(=O)(O)O] | (xxxx) |
| 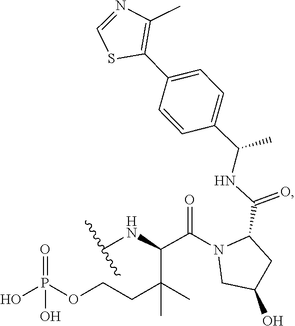 [SMILES: *N[C@@H](C(=O)N1C[C@H](O)C[C@H]1C(=O)N[C@@H](C)c1ccc(-c2scnc2C)cc1)C(C)(C)CCOP(=O)(O)O] | (yyyy) |
| 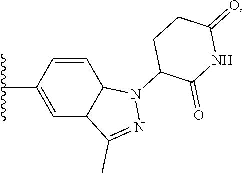 [SMILES: *C1=CC2C(C)=NN(C3CCC(=O)NC3=O)C2C=C1] | (zzzz) |
| 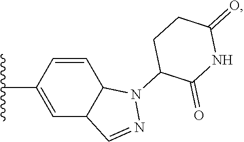 [SMILES: *C1=CC2C=NN(C3CCC(=O)NC3=O)C2C=C1] | (aaaaa) |
| 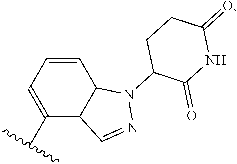 [SMILES: *C1=CC=CC2C1C=NN2C1CCC(=O)NC1=O] | (bbbbb) |
| 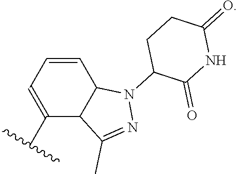 [SMILES: *C1=CC=CC2C1C(C)=NN2C1CCC(=O)NC1=O] | (ccccc) |

TABLE B
Exemplified Linkers (L)

| | |
|---|---|
| 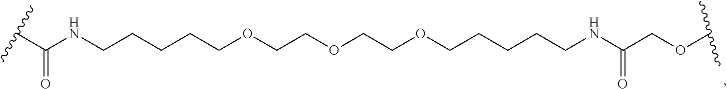 [SMILES: *OCC(=O)NCCCCCOCCOCCOCCCCCNC(*)=O] | (1) |
| 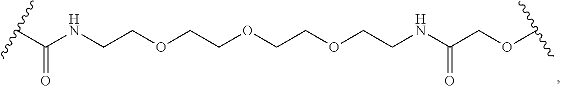 [SMILES: *OCC(=O)NCCOCCOCCOCCNC(*)=O] | (2) |
| 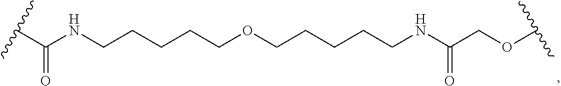 [SMILES: *OCC(=O)NCCCCCOCCCCCNC(*)=O] | (3) |
| 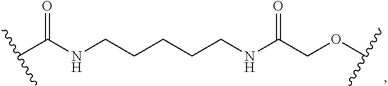 [SMILES: *OCC(=O)NCCCCCNC(*)=O] | (4) |
| 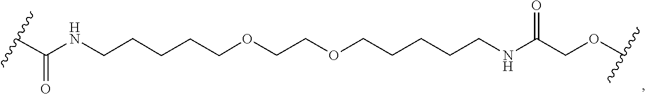 [SMILES: *OCC(=O)NCCCCCOCCOCCCCCNC(*)=O] | (5) |
| 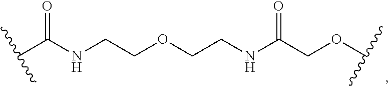 [SMILES: *OCC(=O)NCCOCCNC(*)=O] | (6) |
| 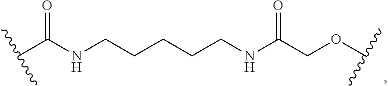 [SMILES: *OCC(=O)NCCCCCNC(*)=O] | (7) |
| 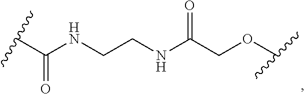 [SMILES: *OCC(=O)NCCNC(*)=O] | (8) |
| 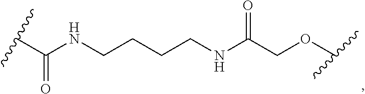 [SMILES: *OCC(=O)NCCCCNC(*)=O] | (9) |
| 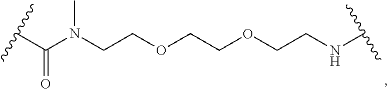 [SMILES: *NCCOCCOCCN(C)C(*)=O] | (10) |
| 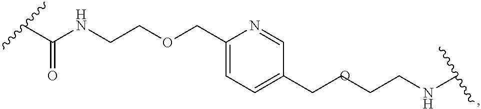 | (11) |
| 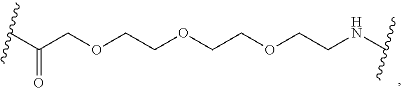 [SMILES: *NCCOCCOCCOCC(*)=O] | (12) |
| 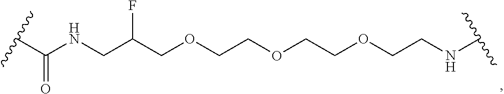 [SMILES: *NCCOCCOCCOCC(F)CNC(*)=O] | (13) |
| 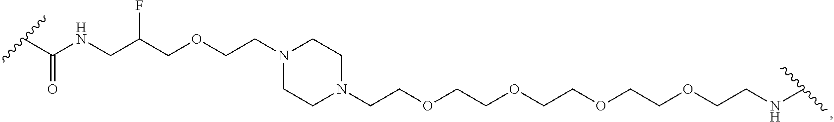 [SMILES: *NCCOCCOCCOCCOCCN1CCN(CCOCC(F)CNC(*)=O)CC1] | (14) |
| 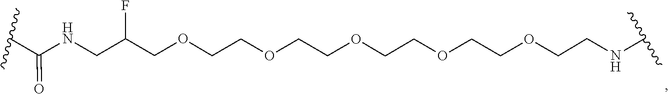 [SMILES: *NCCOCCOCCOCCOCCOCC(F)CNC(*)=O] | (15) |
| 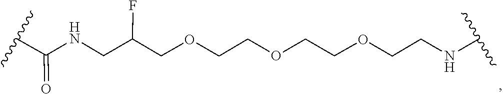 [SMILES: *NCCOCCOCCOCC(F)CNC(*)=O] | (16) |
| 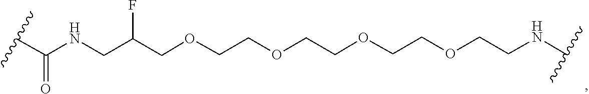 [SMILES: *NCCOCCOCCOCCOCC(F)CNC(*)=O] | (17) |
| 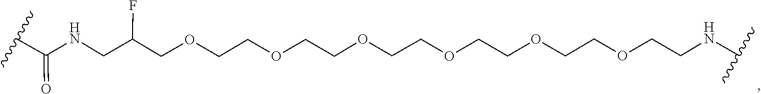 [SMILES: *NCCOCCOCCOCCOCCOCCOCC(F)CNC(*)=O] | (18) |
| 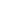 | (19) |
| 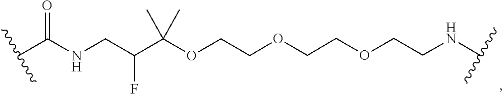 [SMILES: *NCCOCCOCCOC(C)(C)C(F)CNC(*)=O] | (20) |
| 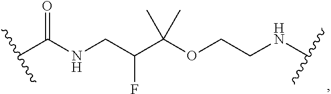 [SMILES: *NCCOC(C)(C)C(F)CNC(*)=O] | (21) |
| 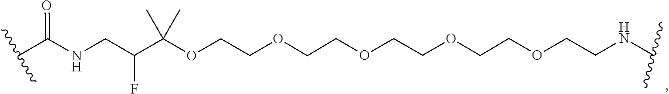 [SMILES: *NCCOCCOCCOCCOCCOC(C)(C)C(F)CNC(*)=O] | (22) |
| 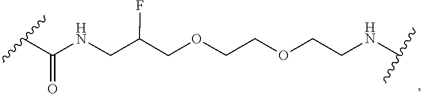 [SMILES: *NCCOCCOCC(F)CNC(*)=O] | (23) |
| 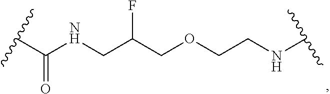 [SMILES: *NCCOCC(F)CNC(*)=O] | (24) |
| 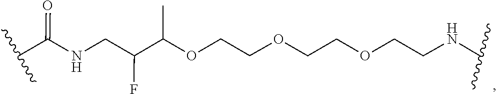 [SMILES: *NCCOCCOCCOC(C)C(F)CNC(*)=O] | (25) |
| 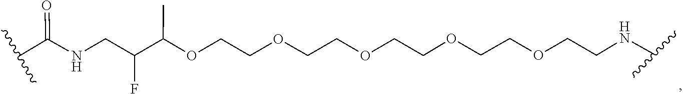 [SMILES: *NCCOCCOCCOCCOCCOC(C)C(F)CNC(*)=O] | (26) |
| 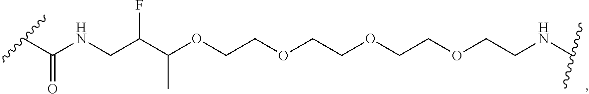 [SMILES: *NCCOCCOCCOCCOC(C)C(F)CNC(*)=O] | (27) |
| 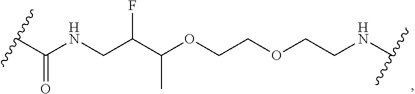 [SMILES: *NCCOCCOC(C)C(F)CNC(*)=O] | (28) |
| 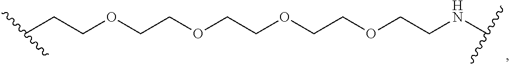 [SMILES: *CCOCCOCCOCCOCCN*] | (29) |
| 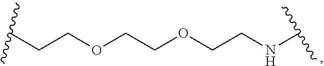 [SMILES: *CCOCCOCCN*] | (30) |
| 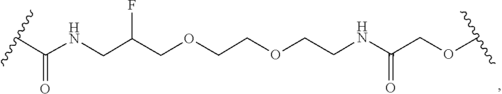 [SMILES: *OCC(=O)NCCOCCOCC(F)CNC(*)=O] | (31) |
| 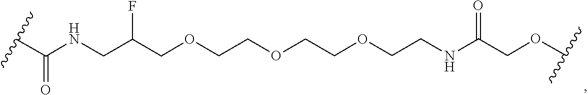 [SMILES: *OCC(=O)NCCOCCOCCOCC(F)CNC(*)=O] | (32) |
| 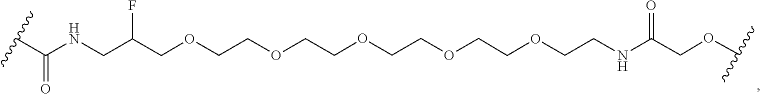 [SMILES: *OCC(=O)NCCOCCOCCOCCOCCOCC(F)CNC(*)=O] | (33) |
| 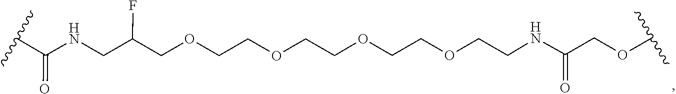 [SMILES: *OCC(=O)NCCOCCOCCOCCOCC(F)CNC(*)=O] | (34) |
| 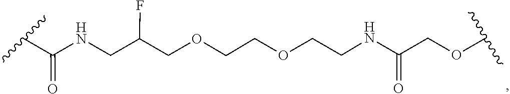 [SMILES: *OCC(=O)NCCOCCOCC(F)CNC(*)=O] | (35) |
| 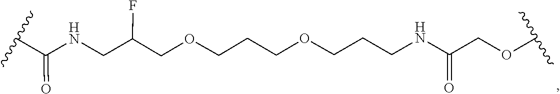 [SMILES: *OCC(=O)NCCCOCCCOCC(F)CNC(*)=O] | (36) |
| 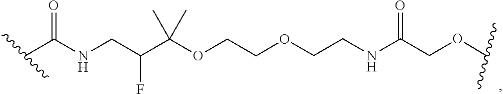 [SMILES: *OCC(=O)NCCOCCOC(C)(C)C(F)CNC(*)=O] | (37) |
| 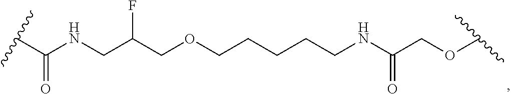 [SMILES: *OCC(=O)NCCCCCOCC(F)CNC(*)=O] | (38) |
| 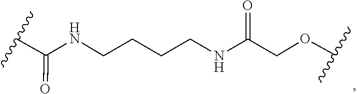 [SMILES: *OCC(=O)NCCCCNC(*)=O] | (39) |
| 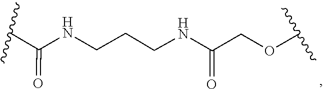 [SMILES: *OCC(=O)NCCCNC(*)=O] | (40) |
| 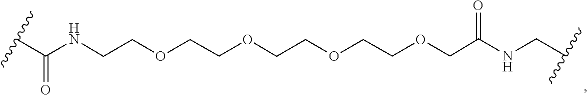 [SMILES: *CNC(=O)COCCOCCOCCOCCNC(*)=O] | (41) |
| 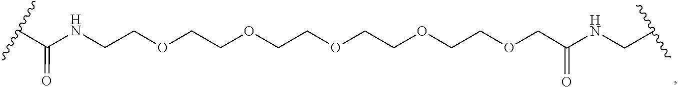 [SMILES: *CNC(=O)COCCOCCOCCOCCOCCNC(*)=O] | (42) |
| 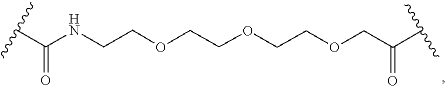 [SMILES: *C(=O)COCCOCCOCCNC(*)=O] | (43) |
| 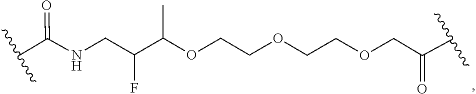 [SMILES: *C(=O)COCCOCCOC(C)C(F)CNC(*)=O] | (44) |
| 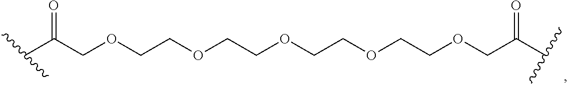 [SMILES: *C(=O)COCCOCCOCCOCCOCC(*)=O] | (45) |
| 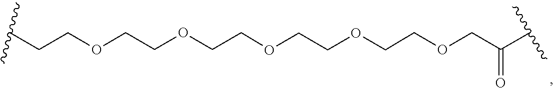 [SMILES: *CCOCCOCCOCCOCCOCC(*)=O] | (46) |
| 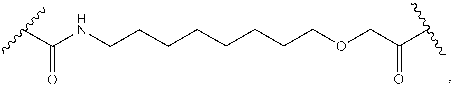 [SMILES: *C(=O)COCCCCCCCCNC(*)=O] | (47) |
| 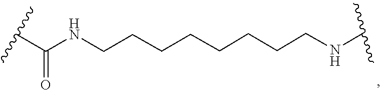 [SMILES: *NCCCCCCCCNC(*)=O] | (49) |
| 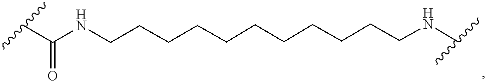 [SMILES: *NCCCCCCCCCCCNC(*)=O] | (50) |
| 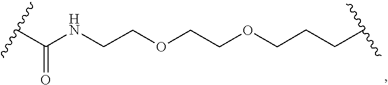 [SMILES: *CCCOCCOCCNC(*)=O] | (51) |
| 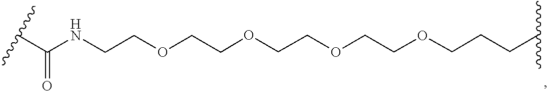 [SMILES: *CCCOCCOCCOCCOCCNC(*)=O] | (52) |
| 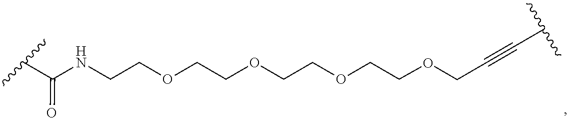 [SMILES: *C#CCOCCOCCOCCOCCNC(*)=O] | (53) |
| 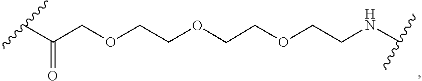 [SMILES: *NCCOCCOCCOCC(*)=O] | (54) |
| 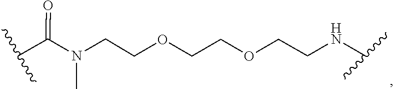 [SMILES: *NCCOCCOCCN(C)C(*)=O] | (55) |
| 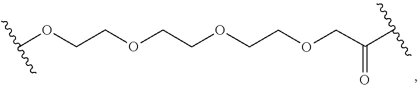 [SMILES: *OCCOCCOCCOCC(*)=O] | (56) |
| 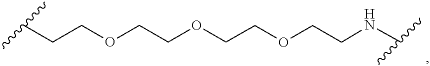 [SMILES: *CCOCCOCCOCCN*] | (57) |
| 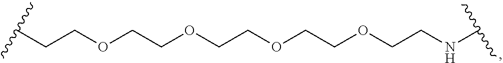 [SMILES: *CCOCCOCCOCCOCCN*] | (58) |
| 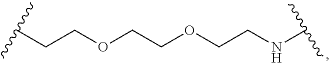 [SMILES: *CCOCCOCCN*] | (59) |
| 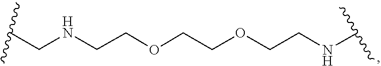 [SMILES: *CNCCOCCOCCN*] | (60) |
| 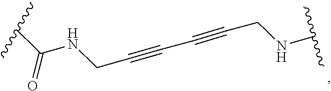 [SMILES: *NCC#CC#CCNC(*)=O] | (61) |
| 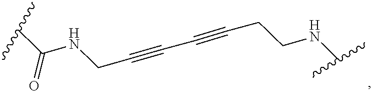 [SMILES: *NCCC#CC#CCNC(*)=O] | (62) |
| 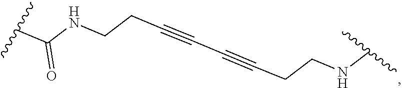 [SMILES: *NCCC#CC#CCCNC(*)=O] | (63) |
| 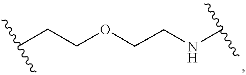 [SMILES: *CCOCCN*] | (64) |
| 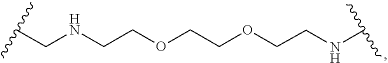 [SMILES: *CNCCOCCOCCN*] | (65) |
| 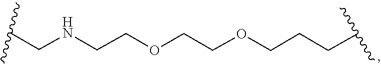 [SMILES: *CCCOCCOCCNC*] | (66) |
| 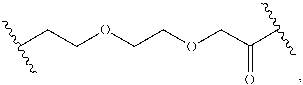 [SMILES: *CCOCCOCC(*)=O] | (67) |
| 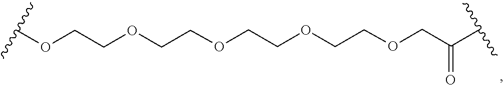 [SMILES: *OCCOCCOCCOCCOCC(*)=O] | (68) |
| 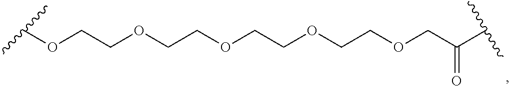 [SMILES: *OCCOCCOCCOCCOCC(*)=O] | (69) |
| 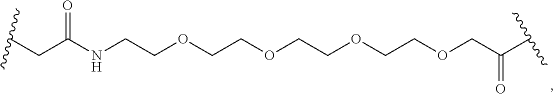 [SMILES: *CC(=O)NCCOCCOCCOCCOCC(*)=O] | (70) |
| 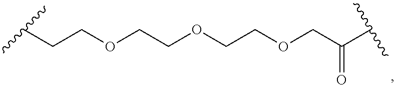 [SMILES: *CCOCCOCCOCC(*)=O] | (71) |
| 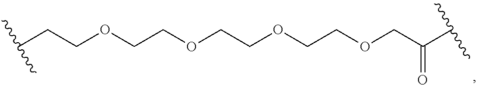 [SMILES: *CCOCCOCCOCCOCC(*)=O] | (72) |
| 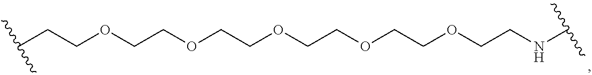 [SMILES: *CCOCCOCCOCCOCCOCCN*] | (73) |
| 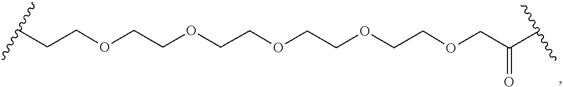 [SMILES: *CCOCCOCCOCCOCCOCC(*)=O] | (74) |
| 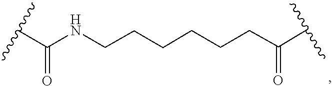 [SMILES: *C(=O)CCCCCCNC(*)=O] | (75) |
| 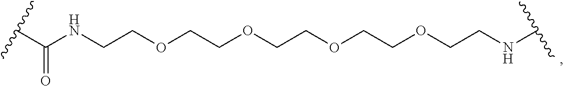 [SMILES: *NCCOCCOCCOCCOCCNC(*)=O] | (76) |
| 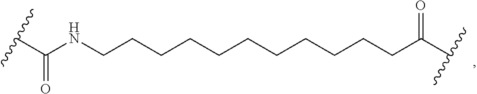 [SMILES: *C(=O)CCCCCCCCCCCNC(*)=O] | (77) |
| 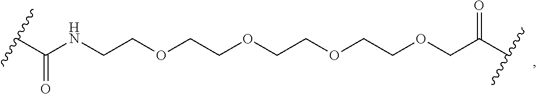 [SMILES: *C(=O)COCCOCCOCCOCCNC(*)=O] | (78) |
| 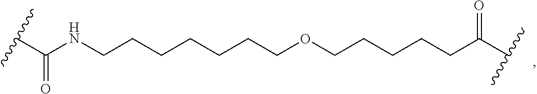 [SMILES: *C(=O)CCCCCOCCCCCCCNC(*)=O] | (79) |
| 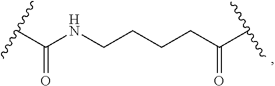 [SMILES: *C(=O)CCCCNC(*)=O] | (80) |
| 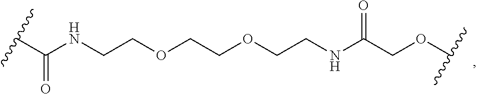 [SMILES: *OCC(=O)NCCOCCOCCNC(*)=O] | (81) |
| 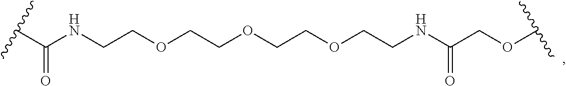 [SMILES: *OCC(=O)NCCOCCOCCOCCNC(*)=O] | (82) |
| 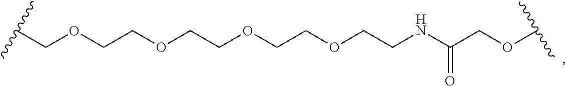 [SMILES: *COCCOCCOCCOCCNC(=O)CO*] | (83) |
| 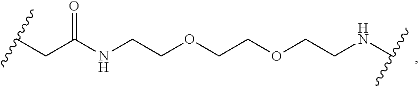 [SMILES: *CC(=O)NCCOCCOCCN*] | (84) |
| 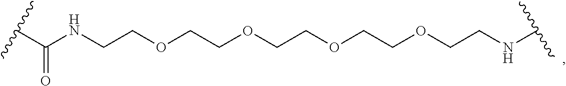 [SMILES: *NCCOCCOCCOCCOCCNC(*)=O] | (85) |
| 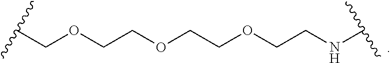 [SMILES: *COCCOCCOCCN*] | (86) |
| 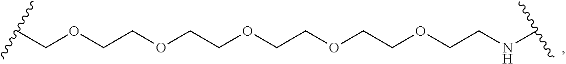 [SMILES: *COCCOCCOCCOCCOCCN*] | (87) |
| 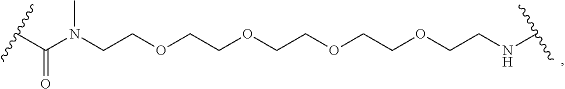 [SMILES: *NCCOCCOCCOCCOCCN(C)C(*)=O] | (88) |
| 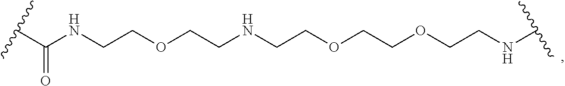 [SMILES: *NCCOCCOCCNCCOCCNC(*)=O] | (89) |
| 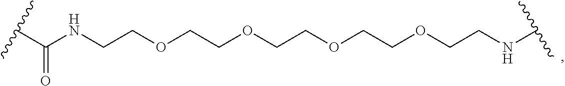 [SMILES: *NCCOCCOCCOCCOCCNC(*)=O] | (90) |
| 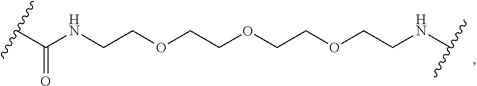 [SMILES: *NCCOCCOCCOCCNC(*)=O] | (91) |
| 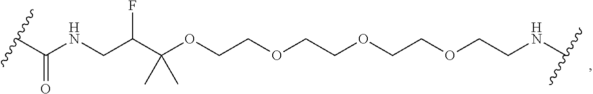 [SMILES: *NCCOCCOCCOCCOC(C)(C)C(F)CNC(*)=O] | (92) |
| 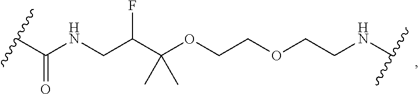 [SMILES: *NCCOCCOC(C)(C)C(F)CNC(*)=O] | (93) |
| 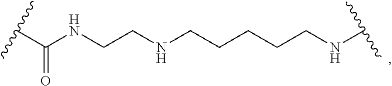 [SMILES: *NCCCCCNCCNC(*)=O] | (94) |
| 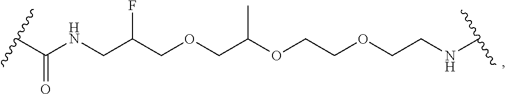 [SMILES: *NCCOCCOC(C)COCC(F)CNC(*)=O] | (95) |
| 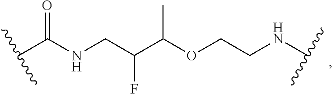 [SMILES: *NCCOC(C)C(F)CNC(*)=O] | (96) |
| 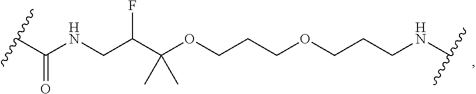 [SMILES: *NCCCOCCCOC(C)(C)C(F)CNC(*)=O] | (97) |
| 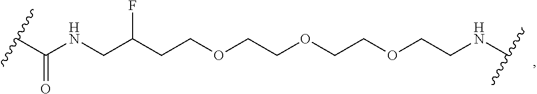 [SMILES: *NCCOCCOCCOCCC(F)CNC(*)=O] | (98) |
| 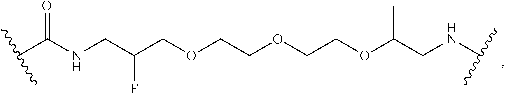 [SMILES: *NCC(C)OCCOCCOCC(F)CNC(*)=O] | (99) |
| 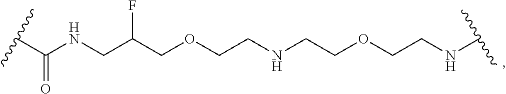 [SMILES: *NCCOCCNCCOCC(F)CNC(*)=O] | (100) |
| 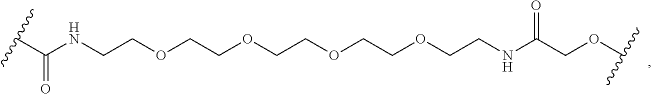 [SMILES: *OCC(=O)NCCOCCOCCOCCOCCNC(*)=O] | (101) |
| 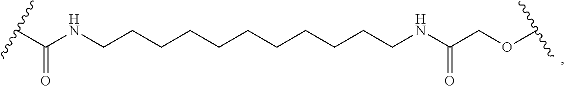 [SMILES: *OCC(=O)NCCCCCCCCCCCNC(*)=O] | (102) |
| 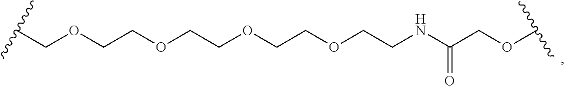 [SMILES: *COCCOCCOCCOCCNC(=O)CO*] | (103) |
| 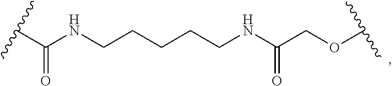 [SMILES: *OCC(=O)NCCCCCNC(*)=O] | (104) |
| 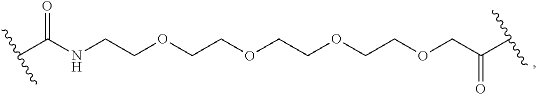 [SMILES: *C(=O)COCCOCCOCCOCCNC(*)=O] | (105) |
| 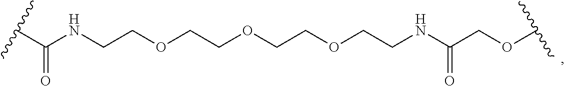 [SMILES: *OCC(=O)NCCOCCOCCOCCNC(*)=O] | (106) |
| 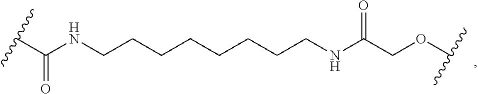 [SMILES: *OCC(=O)NCCCCCCCCNC(*)=O] | (107) |
| 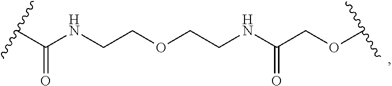 [SMILES: *OCC(=O)NCCOCCNC(*)=O] | (108) |
| 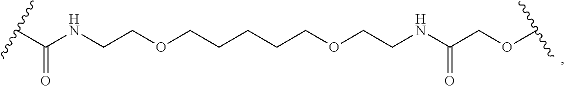 [SMILES: *OCC(=O)NCCOCCCCCOCCNC(*)=O] | (109) |
| 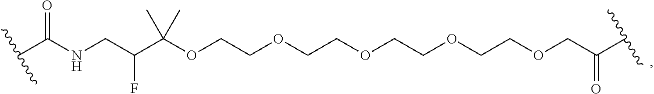 [SMILES: *C(=O)COCCOCCOCCOCCOC(C)(C)C(F)CNC(*)=O] | (110) |
| 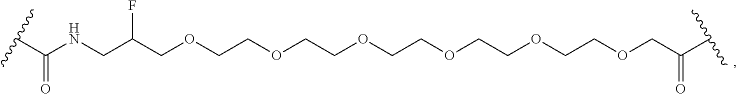 [SMILES: *C(=O)COCCOCCOCCOCCOCCOCC(F)CNC(*)=O] | (111) |
| 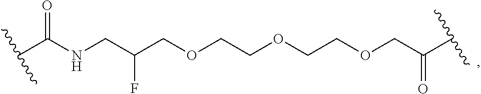 [SMILES: *C(=O)COCCOCCOCC(F)CNC(*)=O] | (112) |
| 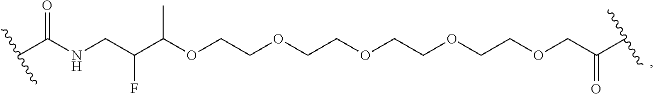 [SMILES: *C(=O)COCCOCCOCCOCCOC(C)C(F)CNC(*)=O] | (113) |
| 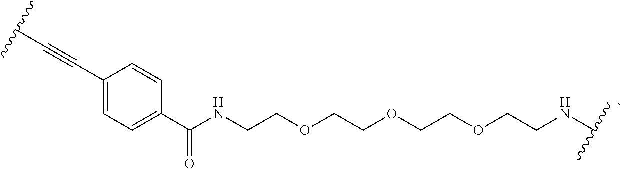 [SMILES: *C#Cc1ccc(C(=O)NCCOCCOCCOCCN*)cc1] | (114) |
| 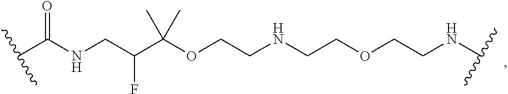 [SMILES: *NCCOCCNCCOC(C)(C)C(F)CNC(*)=O] | (115) |
| 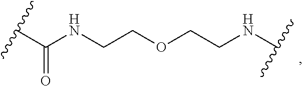 [SMILES: *NCCOCCNC(*)=O] | (116) |
| 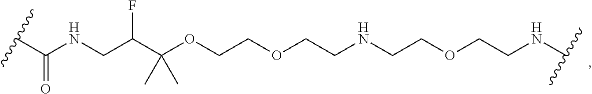 [SMILES: *NCCOCCNCCOCCOC(C)(C)C(F)CNC(*)=O] | (117) |
| 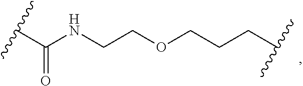 [SMILES: *CCCOCCNC(*)=O] | (118) |
| 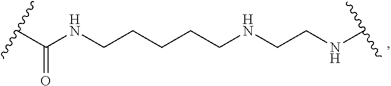 [SMILES: *NCCNCCCCCNC(*)=O] | |
| 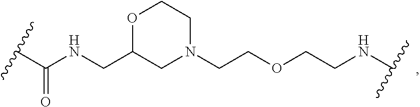 [SMILES: *NCCOCCN1CCOC(CNC(*)=O)C1] | (119) |
| 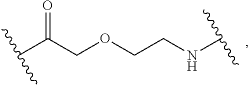 [SMILES: *NCCOCC(*)=O] | |
| 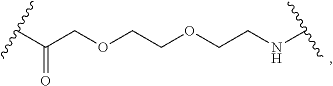 [SMILES: *NCCOCCOCC(*)=O] | (120) |
| 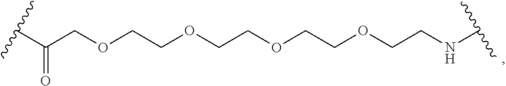 [SMILES: *NCCOCCOCCOCCOCC(*)=O] | (121) |
| 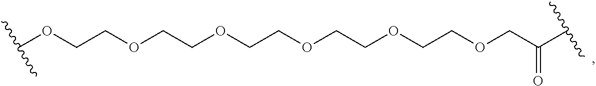 [SMILES: *OCCOCCOCCOCCOCCOCC(*)=O] | (122) |
| 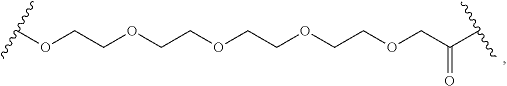 [SMILES: *OCCOCCOCCOCCOCC(*)=O] | (123) |
| 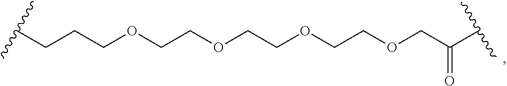 [SMILES: *CCCOCCOCCOCCOCC(*)=O] | (124) |
| 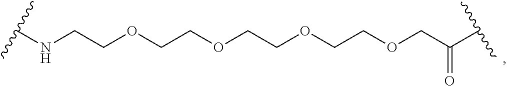 [SMILES: *NCCOCCOCCOCCOCC(*)=O] | (125) |
| 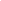 | (126) |
| 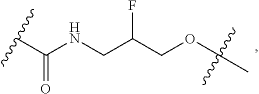 [SMILES: *OCC(F)CNC(*)=O] | |
| 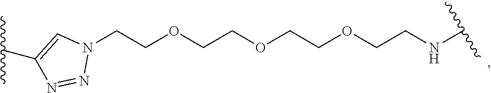 [SMILES: *NCCOCCOCCOCCn1cc(*)nn1] | (127) |
| 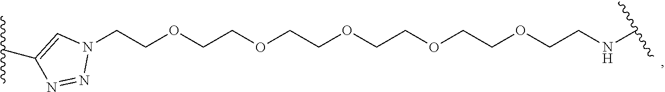 [SMILES: *NCCOCCOCCOCCOCCOCCn1cc(*)nn1] | (128) |
| 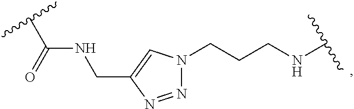 [SMILES: *NCCCn1cc(CNC(*)=O)nn1] | (129) |
| 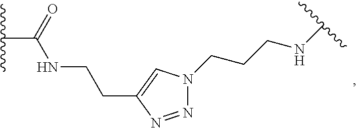 [SMILES: *NCCCn1cc(CCNC(*)=O)nn1] | (130) |
| 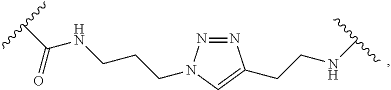 [SMILES: *NCCc1cn(CCCNC(*)=O)nn1] | (131) |
| 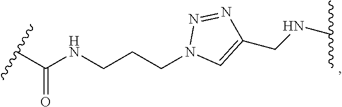 [SMILES: *NCc1cn(CCCNC(*)=O)nn1] | (132) |
| 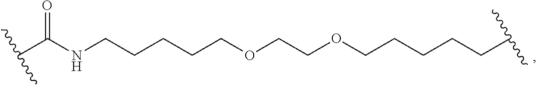 [SMILES: *CCCCCOCCOCCCCCNC(*)=O] | (133) |
| 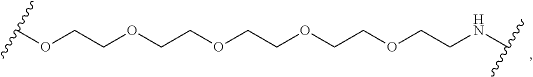 [SMILES: *NCCOCCOCCOCCOCCO*] | (134) |
| 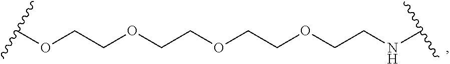 [SMILES: *NCCOCCOCCOCCO*] | (135) |
| 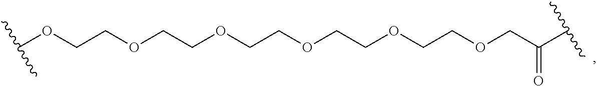 [SMILES: *OCCOCCOCCOCCOCCOCC(*)=O] | (136) |
| 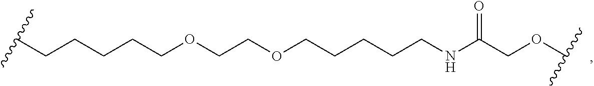 [SMILES: *CCCCCOCCOCCCCCNC(=O)CO*] | (137) |
| 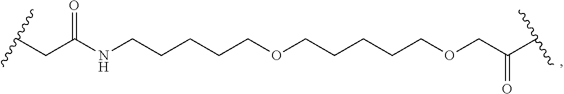 [SMILES: *CC(=O)NCCCCCOCCCCCOCC(*)=O] | (138) |
| 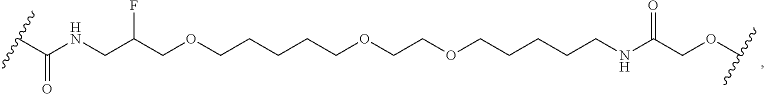 [SMILES: *OCC(=O)NCCCCCOCCOCCCCCOCC(F)CNC(*)=O] | (139) |
| 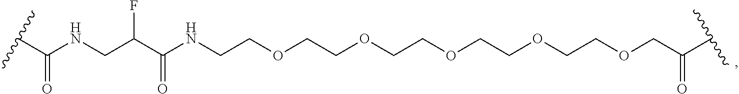 [SMILES: *C(=O)COCCOCCOCCOCCOCCNC(=O)C(F)CNC(*)=O] | (140) |
| 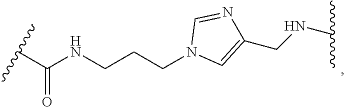 [SMILES: *NCc1cn(CCCNC(*)=O)cn1] | (141) |
| 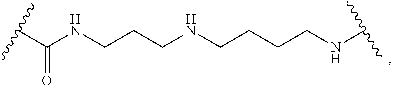 [SMILES: *NCCCCNCCCNC(*)=O] | (142) |
| 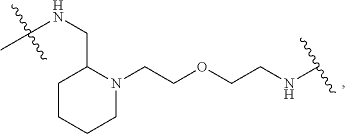 [SMILES: *NCCOCCN1CCCCC1CN*] | (143) |
| 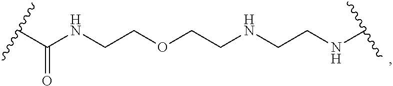 [SMILES: *NCCNCCOCCNC(*)=O] | (144) |
| 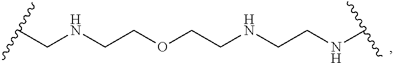 [SMILES: *CNCCOCCNCCN*] | (145) |
| 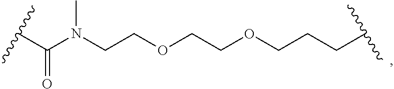 [SMILES: *CCCOCCOCCN(C)C(*)=O] | (146) |
| 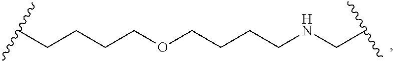 [SMILES: *CCCCOCCCCNC*] | (147) |
| 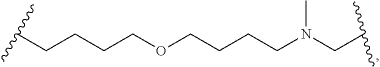 [SMILES: *CCCCOCCCCN(C)C*] | (148) |
| 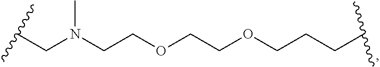 [SMILES: *CCCOCCOCCN(C)C*] | (149) |
| 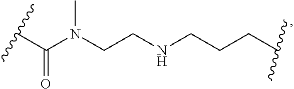 [SMILES: *CCCNCCN(C)C(*)=O] | (150) |
| 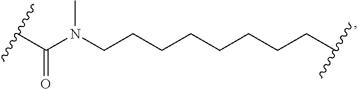 [SMILES: *CCCCCCCCN(C)C(*)=O] | (151) |
| 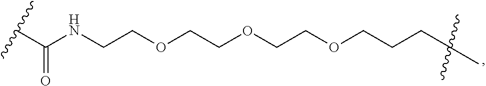 [SMILES: *CCCOCCOCCOCCNC(*)=O] | (152) |
| 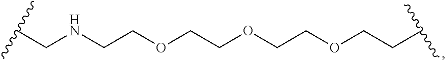 [SMILES: *CCOCCOCCOCCNC*] | (153) |
| 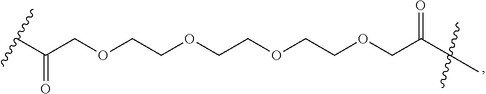 [SMILES: *C(=O)COCCOCCOCCOCC(*)=O] | (154) |
| 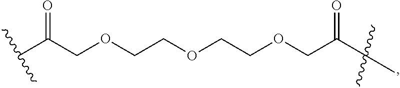 [SMILES: *C(=O)COCCOCCOCC(*)=O] | (155) |
| 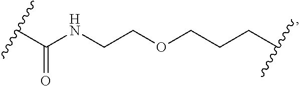 [SMILES: *CCCOCCNC(*)=O] | (156) |
| 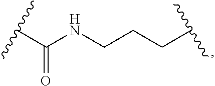 [SMILES: *CCCNC(*)=O] | (157) |
| 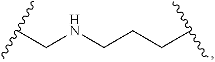 [SMILES: *CCCNC*] | (158) |
| 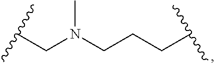 [SMILES: *CCCN(C)C*] | (159) |
| 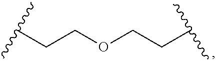 [SMILES: *CCOCC*] | (160) |
| 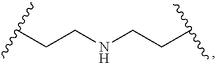 [SMILES: *CCNCC*] | (161) |
| 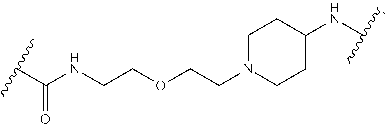 [SMILES: *NC1CCN(CCOCCNC(*)=O)CC1] | (162) |
| 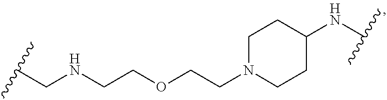 [SMILES: *CNCCOCCN1CCC(N*)CC1] | (163) |
| 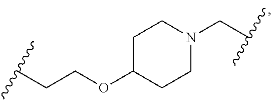 [SMILES: *CCOC1CCN(C*)CC1] | (164) |
| 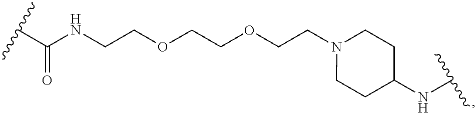 [SMILES: *NC1CCN(CCOCCOCCNC(*)=O)CC1] | (165) |
| 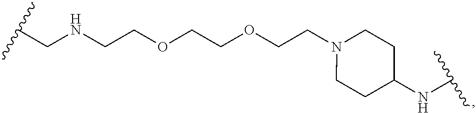 [SMILES: *CNCCOCCOCCN1CCC(N*)CC1] | (166) |
| 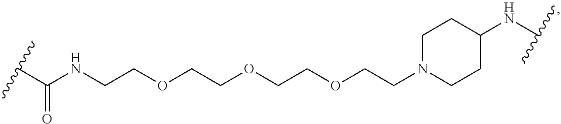 [SMILES: *NC1CCN(CCOCCOCCOCCNC(*)=O)CC1] | (167) |
| 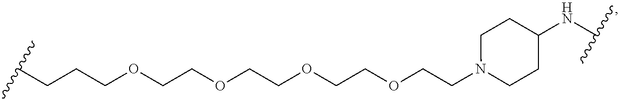 [SMILES: *CCCOCCOCCOCCOCCN1CCC(N*)CC1] | (168) |
| 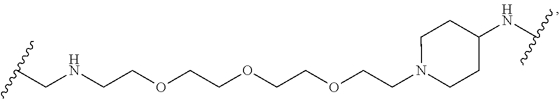 [SMILES: *CNCCOCCOCCOCCN1CCC(N*)CC1] | (169) |
| 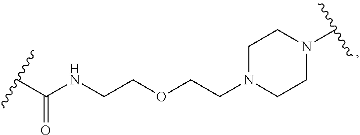 [SMILES: *C(=O)NCCOCCN1CCN(*)CC1] | (170) |
| 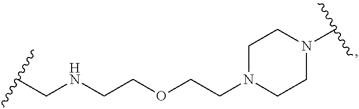 [SMILES: *CNCCOCCN1CCN(*)CC1] | (171) |
| 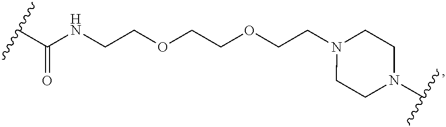 [SMILES: *C(=O)NCCOCCOCCN1CCN(*)CC1] | (172) |
| 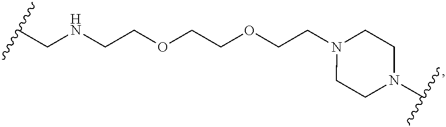 [SMILES: *CNCCOCCOCCN1CCN(*)CC1] | (173) |
| 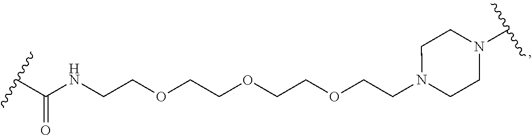 [SMILES: *C(=O)NCCOCCOCCOCCN1CCN(*)CC1] | (174) |
| 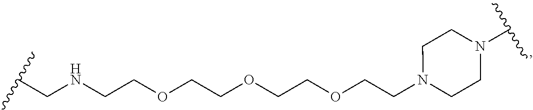 [SMILES: *CNCCOCCOCCOCCN1CCN(*)CC1] | (175) |
| 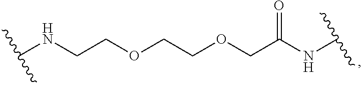 [SMILES: *NCCOCCOCC(=O)N*] | (176) |
| 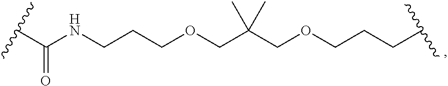 [SMILES: *CCCOCC(C)(C)COCCCNC(*)=O] | (177) |
| 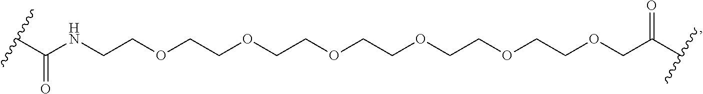 [SMILES: *C(=O)COCCOCCOCCOCCOCCOCCNC(*)=O] | (178) |
| 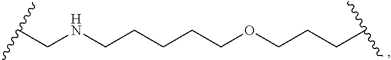 [SMILES: *CCCOCCCCCNC*] | (179) |
| 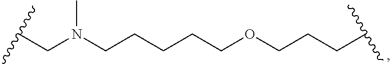 [SMILES: *CCCOCCCCCN(C)C*] | (180) |
| 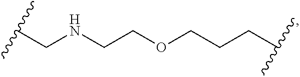 [SMILES: *CCCOCCNC*] | (181) |
| 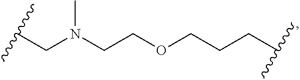 [SMILES: *CCCOCCN(C)C*] | (182) |
| 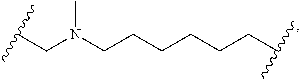 [SMILES: *CCCCCCN(C)C*] | (183) |
| 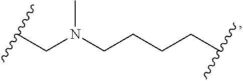 [SMILES: *CCCCN(C)C*] | (184) |
| 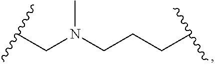 [SMILES: *CCCN(C)C*] | (185) |
| 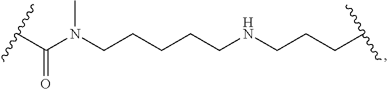 [SMILES: *CCCNCCCCCN(C)C(*)=O] | (186) |
| 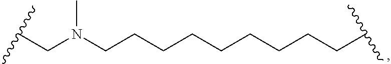 [SMILES: *CCCCCCCCCN(C)C*] | (187) |
| 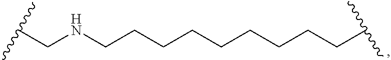 [SMILES: *CCCCCCCCCNC*] | (188) |
| 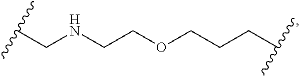 [SMILES: *CCCOCCNC*] | (189) |
| 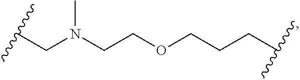 [SMILES: *CCCOCCN(C)C*] | (190) |
| 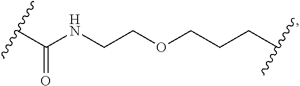 [SMILES: *CCCOCCNC(*)=O] | (191) |
| 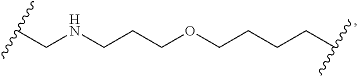 [SMILES: *CCCCOCCCNC*] | (192) |
| 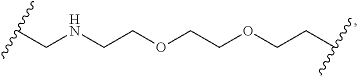 [SMILES: *CCOCCOCCNC*] | (193) |
| 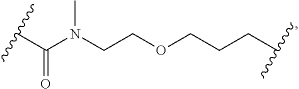 [SMILES: *CCCOCCN(C)C(*)=O] | (194) |
| 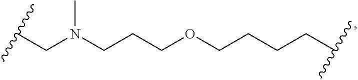 [SMILES: *CCCCOCCCN(C)C*] | (195) |
| 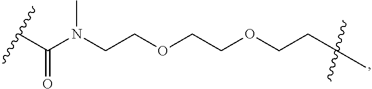 [SMILES: *CCOCCOCCN(C)C(*)=O] | (196) |
| 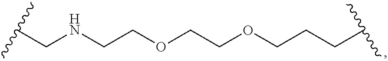 [SMILES: *CCCOCCOCCNC*] | (197) |
| 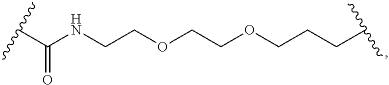 [SMILES: *CCCOCCOCCNC(*)=O] | (198) |
| 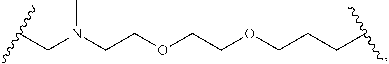 [SMILES: *CCCOCCOCCN(C)C*] | (199) |
| 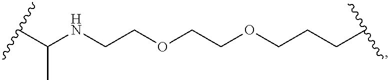 [SMILES: *CCCOCCOCCNC(*)C] | (200) |
| 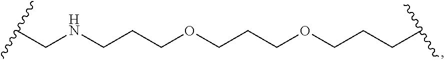 [SMILES: *CCCOCCCOCCCNC*] | (201) |
| 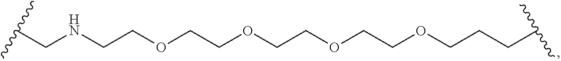 [SMILES: *CCCOCCOCCOCCOCCNC*] | (202) |
| 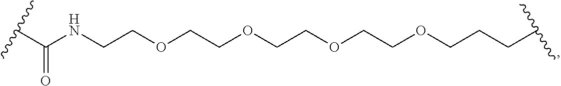 [SMILES: *CCCOCCOCCOCCOCCNC(*)=O] | (203) |
| 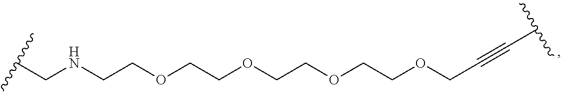 [SMILES: *C#CCOCCOCCOCCOCCNC*] | (204) |
| 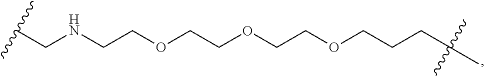 [SMILES: *CCCOCCOCCOCCNC*] | (205) |
| 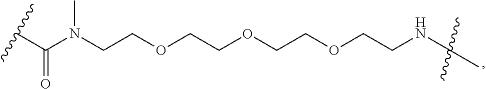 [SMILES: *NCCOCCOCCOCCN(C)C(*)=O] | (206) |
| 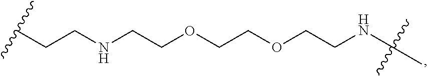 [SMILES: *CCNCCOCCOCCN*] | (207) |
| 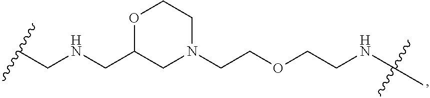 [SMILES: *CNCC1CN(CCOCCN*)CCO1] | (208) |
| 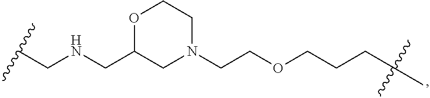 [SMILES: *CCCOCCN1CCOC(CNC*)C1] | (209) |
| 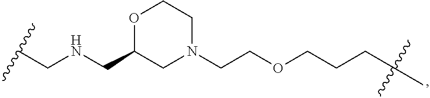 [SMILES: *CCCOCCN1CCO[C@H](CNC*)C1] | (210) |
| 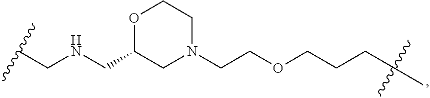 [SMILES: *CCCOCCN1CCO[C@@H](CNC*)C1] | (211) |
| 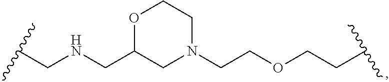 [SMILES: *CCOCCN1CCOC(CNC*)C1] | (212) |
| 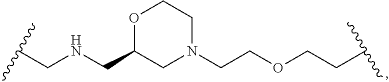 [SMILES: *CCOCCN1CCO[C@H](CNC*)C1] | (213) |
| 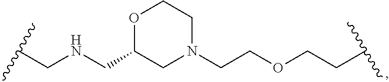 [SMILES: *CCOCCN1CCO[C@@H](CNC*)C1] | (214) |
| 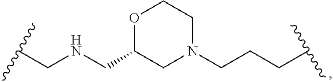 [SMILES: *CCCN1CCO[C@@H](CNC*)C1] | (215) |
| 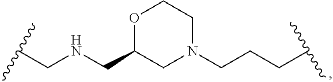 [SMILES: *CCCN1CCO[C@H](CNC*)C1] | (216) |
| 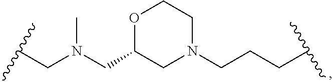 [SMILES: *CCCN1CCO[C@@H](CN(C)C*)C1] | (217) |
| 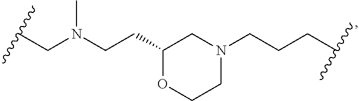 [SMILES: *CCCN1CCO[C@H](CCN(C)C*)C1] | (218) |
| 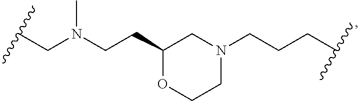 [SMILES: *CCCN1CCO[C@@H](CCN(C)C*)C1] | (219) |
| 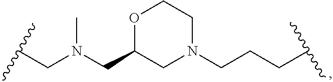 [SMILES: *CCCN1CCO[C@H](CN(C)C*)C1] | (220) |
| 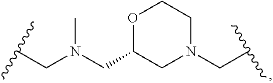 [SMILES: *CN(C)C[C@H]1CN(C*)CCO1] | (221) |
| 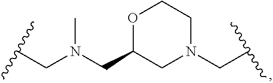 [SMILES: *CN(C)C[C@@H]1CN(C*)CCO1] | (222) |
| 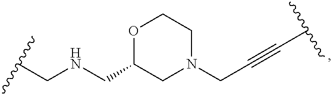 [SMILES: *C#CCN1CCO[C@@H](CNC*)C1] | (223) |
| 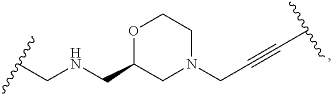 [SMILES: *C#CCN1CCO[C@H](CNC*)C1] | (224) |
| 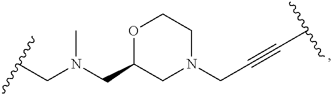 [SMILES: *C#CCN1CCO[C@H](CN(C)C*)C1] | (225) |
| 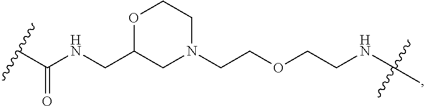 | (226) |
| 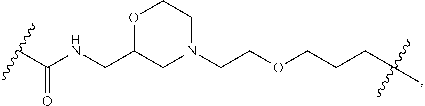 | (227) |
| 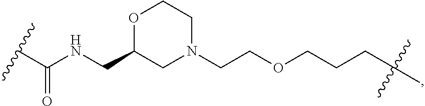 | (228) |
| 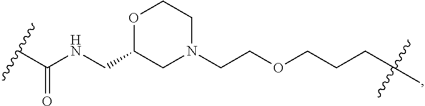 | (229) |
| 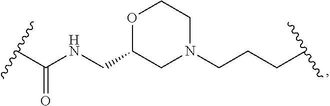 [SMILES: *CCCN1CCO[C@@H](CNC(*)=O)C1] | (230) |
| 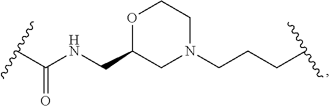 [SMILES: *CCCN1CCO[C@H](CNC(*)=O)C1] | (231) |
| 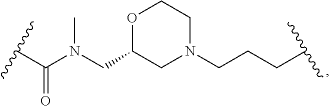 [SMILES: *CCCN1CCO[C@@H](CN(C)C(*)=O)C1] | (232) |
| 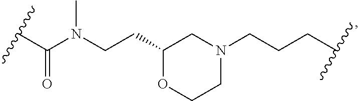 [SMILES: *CCCN1CCO[C@H](CCN(C)C(*)=O)C1] | (233) |
| 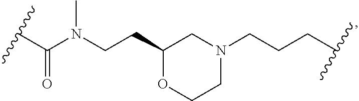 [SMILES: *CCCN1CCO[C@@H](CCN(C)C(*)=O)C1] | (234) |
| 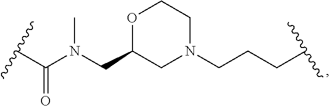 [SMILES: *CCCN1CCO[C@H](CN(C)C(*)=O)C1] | (235) |
| 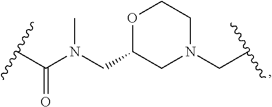 [SMILES: *CN1CCO[C@@H](CN(C)C(*)=O)C1] | (236) |
| 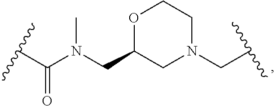 [SMILES: *CN1CCO[C@H](CN(C)C(*)=O)C1] | (237) |
| 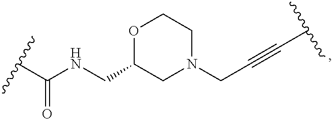 [SMILES: *C#CCN1CCO[C@@H](CNC(*)=O)C1] | (238) |
| 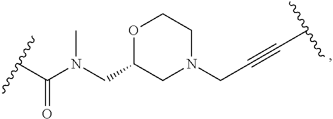 [SMILES: *C#CCN1CCO[C@@H](CN(C)C(*)=O)C1] | (239) |
| 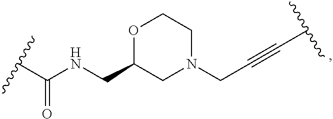 [SMILES: *C#CCN1CCO[C@H](CNC(*)=O)C1] | (240) |
| 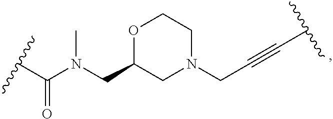 [SMILES: *C#CCN1CCO[C@H](CN(C)C(*)=O)C1] | (241) |
| 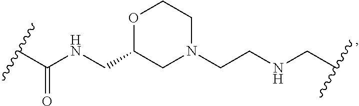 [SMILES: *CNCCN1CCO[C@@H](CNC(*)=O)C1] | (242) |
| 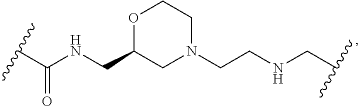 [SMILES: *CNCCN1CCO[C@H](CNC(*)=O)C1] | (243) |
| 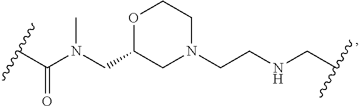 [SMILES: *CNCCN1CCO[C@@H](CN(C)C(*)=O)C1] | (244) |
| 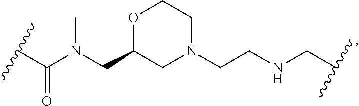 [SMILES: *CNCCN1CCO[C@H](CN(C)C(*)=O)C1] | (245) |
| 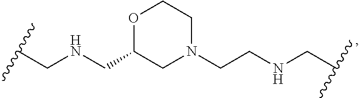 [SMILES: *CNCCN1CCO[C@@H](CNC*)C1] | (246) |
| 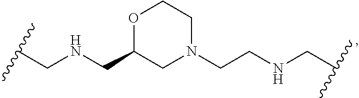 [SMILES: *CNCCN1CCO[C@H](CNC*)C1] | (247) |
| 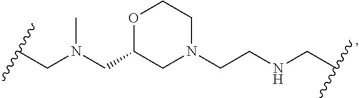 [SMILES: *CNCCN1CCO[C@@H](CN(C)C*)C1] | (248) |
| 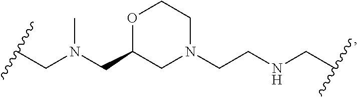 [SMILES: *CNCCN1CCO[C@H](CN(C)C*)C1] | (249) |
| 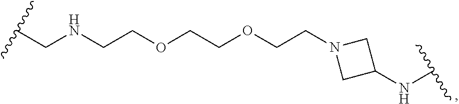 [SMILES: *CNCCOCCOCCN1CC(N*)C1] | (250) |
| 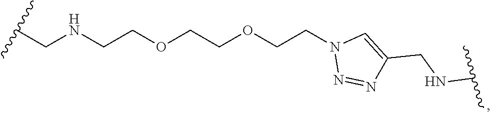 [SMILES: *CNCCOCCOCCn1cc(CN*)nn1] | (251) |
| 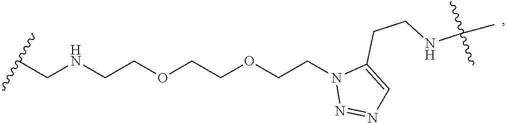 [SMILES: *CNCCOCCOCCn1nncc1CCN*] | (253) |
| 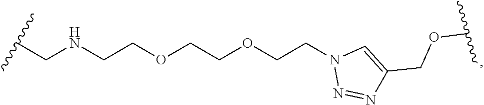 [SMILES: *CNCCOCCOCCn1cc(CO*)nn1] | (254) |
| 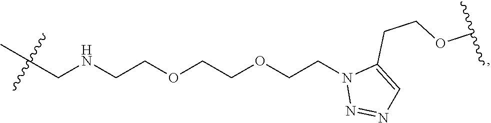 [SMILES: *CNCCOCCOCCn1nncc1CCO*] | (255) |
| 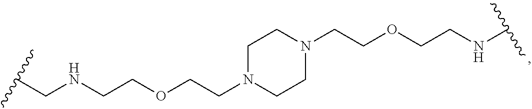 [SMILES: *CNCCOCCN1CCN(CCOCCN*)CC1] | (256) |
| 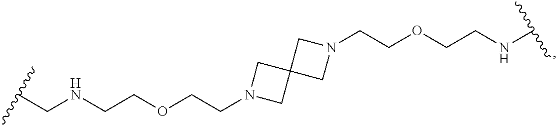 [SMILES: *CNCCOCCN1CC2(CN(CCOCCN*)C2)C1] | (257) |
| 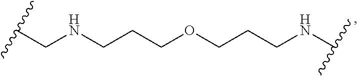 [SMILES: *CNCCCOCCCN*] | (258) |
| 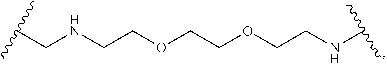 [SMILES: *CNCCOCCOCCN*] | (259) |
| 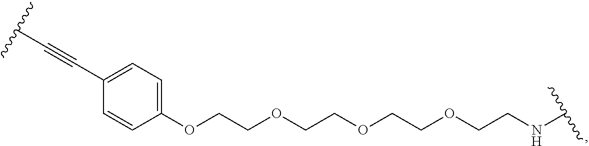 [SMILES: *C#Cc1ccc(OCCOCCOCCOCCN*)cc1] | (260) |
| 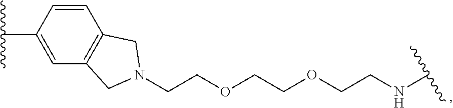 [SMILES: *NCCOCCOCCN1Cc2ccc(*)cc2C1] | (261) |
| 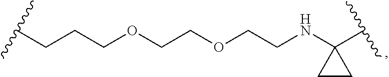 [SMILES: *CCCOCCOCCNC1(*)CC1] | (262) |
| 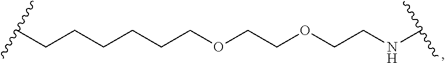 [SMILES: *CCCCCCOCCOCCN*] | (263) |
| 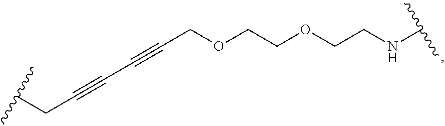 [SMILES: *CC#CC#CCOCCOCCN*] | (264) |
| 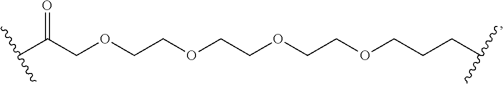 [SMILES: *CCCOCCOCCOCCOCC(*)=O] | (265) |
| 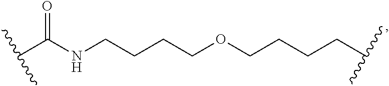 [SMILES: *CCCCOCCCCNC(*)=O] | (266) |
| 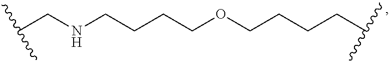 [SMILES: *CCCCOCCCCNC*] | (267) |
| 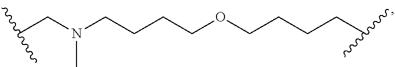 [SMILES: *CCCCOCCCCN(C)C*] | (268) |
| 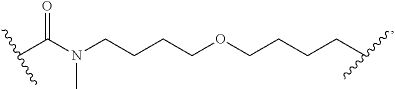 [SMILES: *CCCCOCCCCN(C)C(*)=O] | (269) |
| 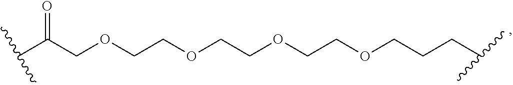 [SMILES: *CCCOCCOCCOCCOCC(*)=O] | (270) |
| 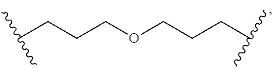 [SMILES: *CCCOCCC*] | (271) |
| 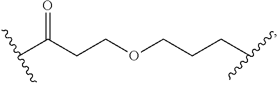 [SMILES: *CCCOCCC(*)=O] | (272) |
| 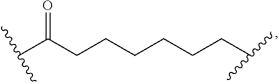 [SMILES: *CCCCCCC(*)=O] | (273) |
| 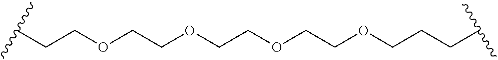 [SMILES: *CCCOCCOCCOCCOCC*] | (274) |
| 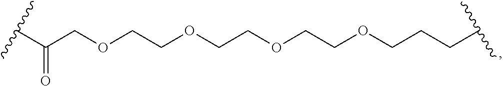 [SMILES: *CCCOCCOCCOCCOCC(*)=O] | (275) |
| 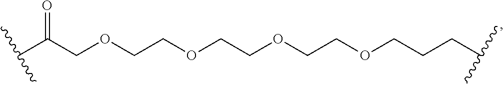 [SMILES: *CCCOCCOCCOCCOCC(*)=O] | (276) |
| 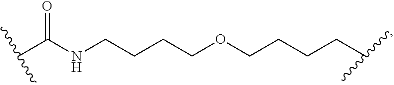 [SMILES: *CCCCOCCCCNC(*)=O] | (277) |
| 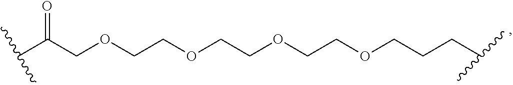 [SMILES: *CCCOCCOCCOCCOCC(*)=O] | (278) |
| 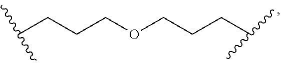 [SMILES: *CCCOCCC*] | (279) |
| 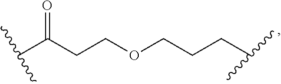 [SMILES: *CCCOCCC(*)=O] | (280) |
| 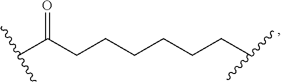 [SMILES: *CCCCCCC(*)=O] | (281) |
| 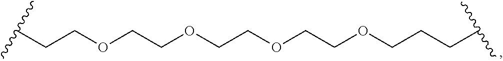 [SMILES: *CCCOCCOCCOCCOCC*] | (282) |
| 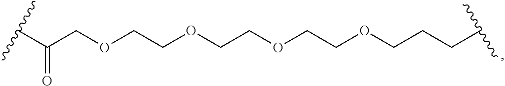 [SMILES: *CCCOCCOCCOCCOCC(*)=O] | (283) |
| 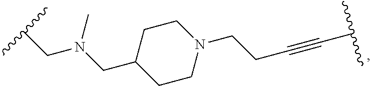 [SMILES: *C#CCCN1CCC(CN(C)C*)CC1] | (284) |
| 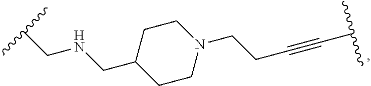 [SMILES: *C#CCCN1CCC(CNC*)CC1] | (285) |
| 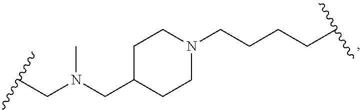 [SMILES: *CCCCN1CCC(CN(C)C*)CC1] | (286) |
| 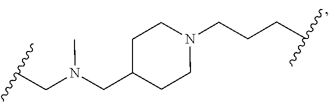 [SMILES: *CCCN1CCC(CN(C)C*)CC1] | (287) |
| 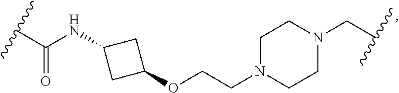 [SMILES: *CN1CCN(CCO[C@H]2C[C@H](NC(*)=O)C2)CC1] | (288) |
| 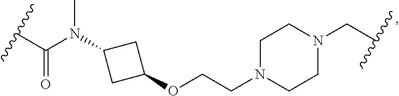 [SMILES: *CN1CCN(CCO[C@H]2C[C@H](N(C)C(*)=O)C2)CC1] | (289) |
| 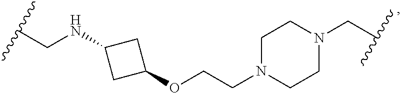 [SMILES: *CN[C@H]1C[C@H](OCCN2CCN(C*)CC2)C1] | (290) |
| 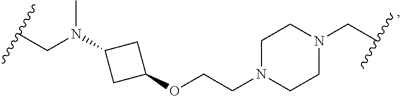 [SMILES: *CN1CCN(CCO[C@H]2C[C@H](N(C)C*)C2)CC1] | (291) |
| 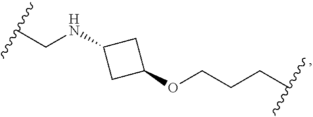 [SMILES: *CCCO[C@H]1C[C@H](NC*)C1] | (292) |
| 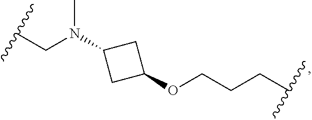 [SMILES: *CCCO[C@H]1C[C@H](N(C)C*)C1] | (293) |
| 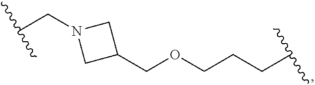 [SMILES: *CCCOCC1CN(C*)C1] | (294) |
| 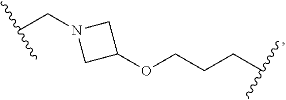 [SMILES: *CCCOC1CN(C*)C1] | (295) |
| 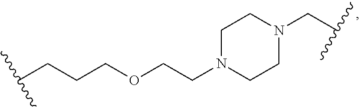 [SMILES: *CCCOCCN1CCN(C*)CC1] | (296) |
| 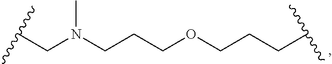 [SMILES: *CCCOCCCN(C)C*] | (297) |
| 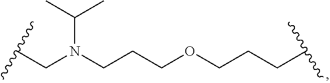 [SMILES: *CCCOCCCN(C*)C(C)C] | (298) |
| 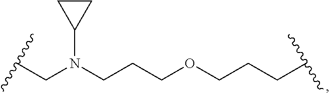 [SMILES: *CCCOCCCN(C*)C1CC1] | (299) |
| 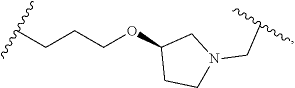 [SMILES: *CCCO[C@@H]1CCN(C*)C1] | (300) |
| 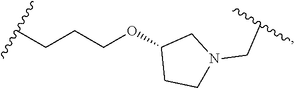 [SMILES: *CCCO[C@H]1CCN(C*)C1] | (301) |
| 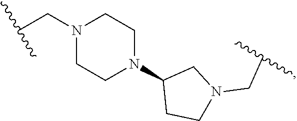 [SMILES: *CN1CCN([C@@H]2CCN(C*)C2)CC1] | (302) |
| 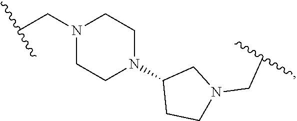 [SMILES: *CN1CCN([C@H]2CCN(C*)C2)CC1] | (303) |
| 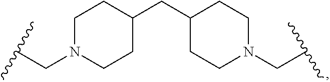 [SMILES: *CN1CCC(CC2CCN(C*)CC2)CC1] | (304) |
| 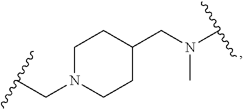 [SMILES: *CN1CCC(CN(*)C)CC1] | (305) |
| 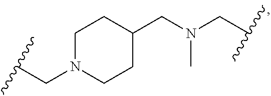 [SMILES: *CN(C)CC1CCN(C*)CC1] | (306) |
| 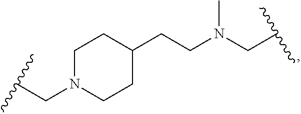 [SMILES: *CN(C)CCC1CCN(C*)CC1] | (307) |
| 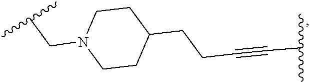 [SMILES: *C#CCCC1CCN(C*)CC1] | (308) |
| 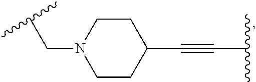 [SMILES: *C#CC1CCN(C*)CC1] | (309) |
| 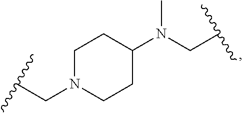 [SMILES: *CN1CCC(N(C)C*)CC1] | (310) |
| 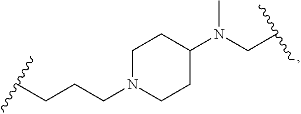 [SMILES: *CCCN1CCC(N(C)C*)CC1] | (311) |
| 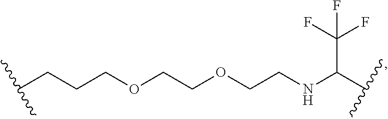 [SMILES: *CCCOCCOCCNC(*)C(F)(F)F] | (312) |
| 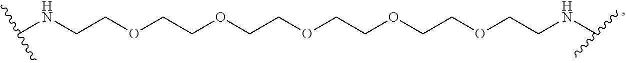 [SMILES: *NCCOCCOCCOCCOCCOCCN*] | (313) |
| 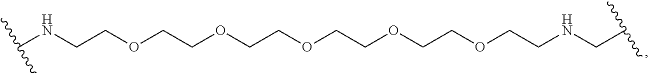 [SMILES: *CNCCOCCOCCOCCOCCOCCN*] | (314) |
| 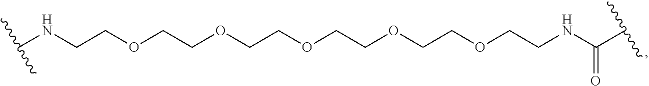 [SMILES: *NCCOCCOCCOCCOCCOCCNC(*)=O] | (315) |
| 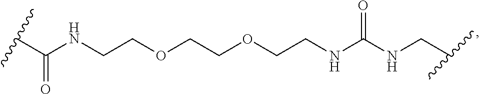 [SMILES: *CNC(=O)NCCOCCOCCNC(*)=O] | (316) |
| 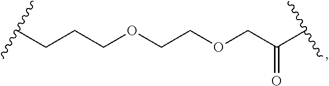 [SMILES: *CCCOCCOCC(*)=O] | (317) |
| 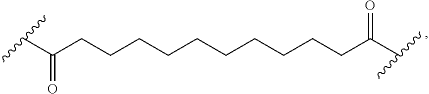 [SMILES: *C(=O)CCCCCCCCCCC(*)=O] | (318) |
| 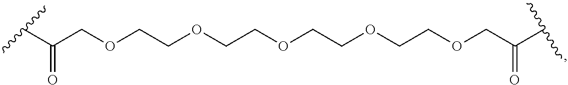 [SMILES: *C(=O)COCCOCCOCCOCCOCC(*)=O] | (319) |
| 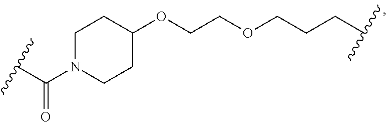 [SMILES: *CCCOCCOC1CCN(C(*)=O)CC1] | (320) |
| 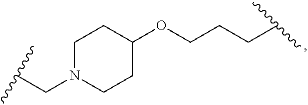 [SMILES: *CCCOC1CCN(C*)CC1] | (321) |
| 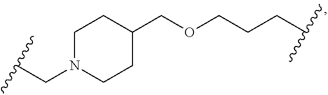 [SMILES: *CCCOCC1CCN(C*)CC1] | (322) |
| 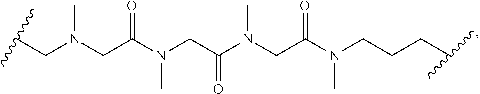 [SMILES: *CCCN(C)C(=O)CN(C)C(=O)CN(C)C(=O)CN(C)C*] | (323) |
| 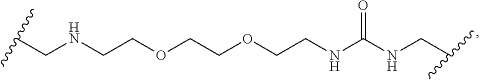 [SMILES: *CNCCOCCOCCNC(=O)NC*] | (324) |
| 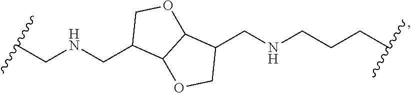 [SMILES: *CCCNCC1COC2C(CNC*)COC12] | (325) |
| 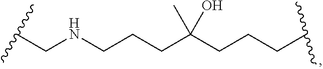 [SMILES: *CCCC(C)(O)CCCNC*] | (326) |
| 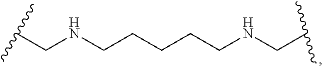 [SMILES: *CNCCCCCNC*] | (327) |
| 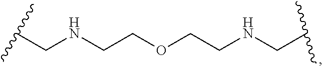 [SMILES: *CNCCOCCNC*] | (328) |
| 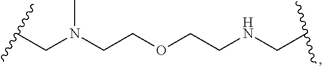 [SMILES: *CNCCOCCN(C)C*] | (329) |
| 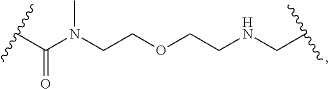 [SMILES: *CNCCOCCN(C)C(*)=O] | (330) |
| 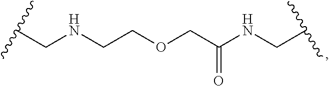 [SMILES: *CNCCOCC(=O)NC*] | (331) |
| 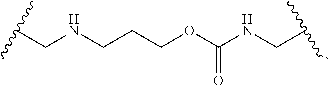 [SMILES: *CNCCCOC(=O)NC*] | (332) |
| 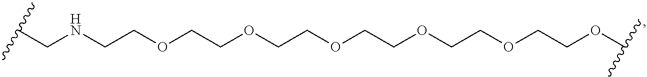 [SMILES: *CNCCOCCOCCOCCOCCOCCO*] | (333) |
| 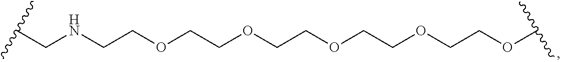 [SMILES: *CNCCOCCOCCOCCOCCO*] | (334) |
| 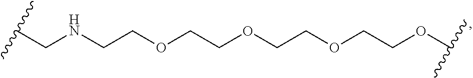 [SMILES: *CNCCOCCOCCOCCO*] | (335) |
| 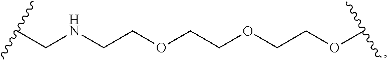 [SMILES: *CNCCOCCOCCO*] | (336) |
| 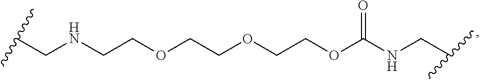 [SMILES: *CNCCOCCOCCOC(=O)NC*] | (337) |
| 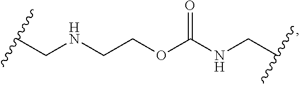 [SMILES: *CNCCOC(=O)NC*] | (338) |
| 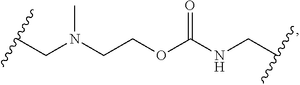 [SMILES: *CNC(=O)OCCN(C)C*] | (339) |
| 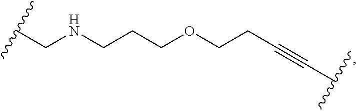 [SMILES: *C#CCCOCCCNC*] | (340) |
| 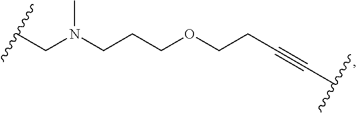 [SMILES: *C#CCCOCCCN(C)C*] | (341) |
| 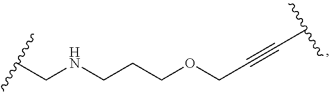 [SMILES: *C#CCOCCCNC*] | (342) |
| 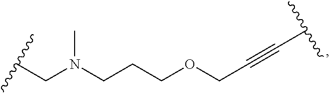 [SMILES: *C#CCOCCCN(C)C*] | (343) |
| 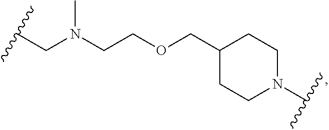 [SMILES: *CN(C)CCOCC1CCN(*)CC1] | (344) |
| 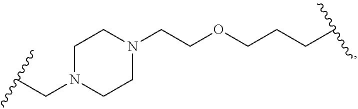 [SMILES: *CCCOCCN1CCN(C*)CC1] | (345) |
| 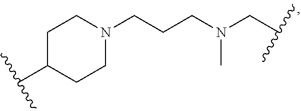 [SMILES: *CN(C)CCCN1CCC(*)CC1] | (346) |
| 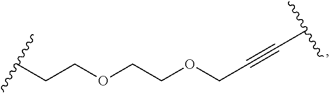 [SMILES: *C#CCOCCOCC*] | (347) |
| 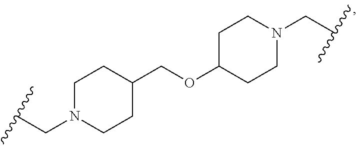 [SMILES: *CN1CCC(COC2CCN(C*)CC2)CC1] | (348) |
| 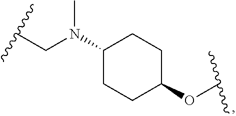 [SMILES: *CN(C)[C@H]1CC[C@H](O*)CC1] | (349) |
| 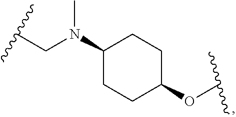 [SMILES: *CN(C)[C@H]1CC[C@@H](O*)CC1] | (350) |
| 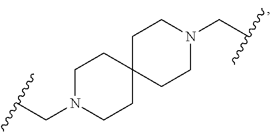 [SMILES: *CN1CCC2(CC1)CCN(C*)CC2] | (351) |
| 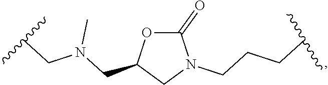 [SMILES: *CCCN1C[C@@H](CN(C)C*)OC1=O] | (352) |
| 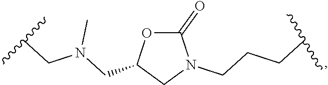 [SMILES: *CCCN1C[C@H](CN(C)C*)OC1=O] | (353) |
| 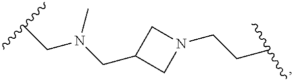 [SMILES: *CCN1CC(CN(C)C*)C1] | (354) |
| 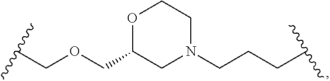 [SMILES: *CCCN1CCO[C@@H](COC*)C1] | (355) |
| 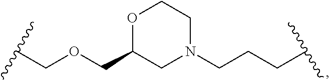 [SMILES: *CCCN1CCO[C@H](COC*)C1] | (356) |
| 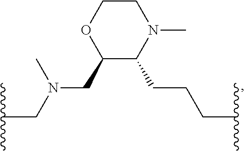 [SMILES: *CCC[C@@H]1[C@@H](CN(C)C*)OCCN1C] | (357) |
| 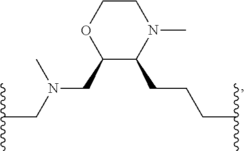 [SMILES: *CCC[C@H]1[C@@H](CN(C)C*)OCCN1C] | (358) |
| 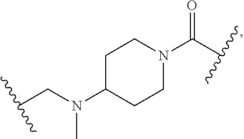 [SMILES: *CN(C)C1CCN(C(*)=O)CC1] | (359) |
| 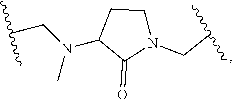 [SMILES: *CN1CCC(N(C)C*)C1=O] | (360) |
| 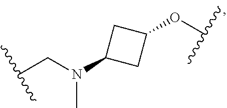 [SMILES: *CN(C)[C@H]1C[C@H](O*)C1] | (361) |
| 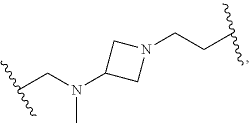 [SMILES: *CCN1CC(N(C)C*)C1] | (362) |
| 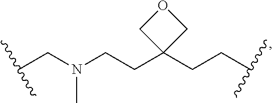 [SMILES: *CCC1(CCN(C)C*)COC1] | (363) |
| 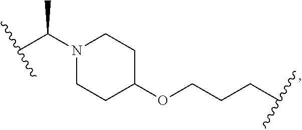 [SMILES: *CCCOC1CCN([C@@H](*)C)CC1] | (364) |
| 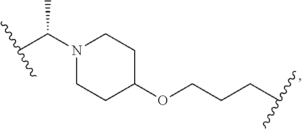 [SMILES: *CCCOC1CCN([C@H](*)C)CC1] | (365) |
| 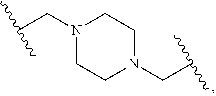 [SMILES: *CN1CCN(C*)CC1] | (366) |
| 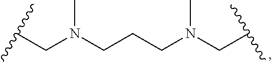 [SMILES: *CN(C)CCCN(C)C*] | (367) |
| 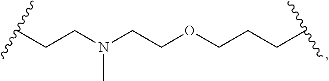 [SMILES: *CCCOCCN(C)CC*] | (368) |
| 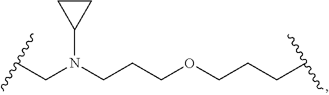 [SMILES: *CCCOCCCN(C*)C1CC1] | (369) |
| 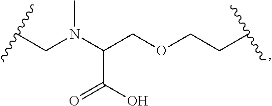 [SMILES: *CCOCC(C(=O)O)N(C)C*] | (370) |
| 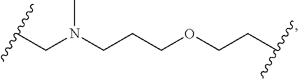 [SMILES: *CCOCCCN(C)C*] | (371) |
| 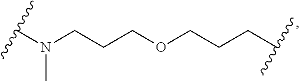 [SMILES: *CCCOCCCN(*)C] | (372) |
| 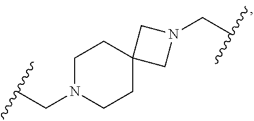 [SMILES: *CN1CCC2(CC1)CN(C*)C2] | (373) |
| 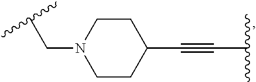 [SMILES: *C#CC1CCN(C*)CC1] | (374) |
| 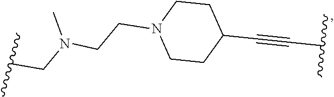 [SMILES: *C#CC1CCN(CCN(C)C*)CC1] | (375) |
| 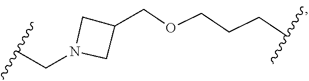 [SMILES: *CCCOCC1CN(C*)C1] | (376) |
| 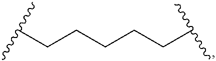 [SMILES: *CCCCC*] | (377) |
| 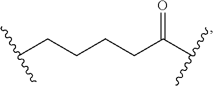 [SMILES: *CCCCC(*)=O] | (378) |
| 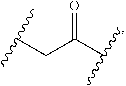 [SMILES: *CC(*)=O] | (379) |
| 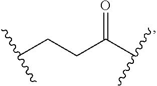 [SMILES: *CCC(*)=O] | (380) |
| 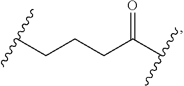 [SMILES: *CCCC(*)=O] | (381) |
| 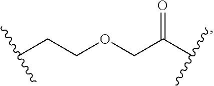 [SMILES: *CCOCC(*)=O] | (382) |
| 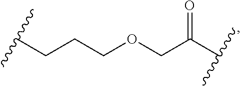 [SMILES: *CCCOCC(*)=O] | (383) |
| 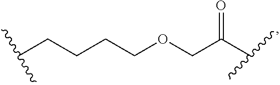 [SMILES: *CCCCOCC(*)=O] | (384) |
| 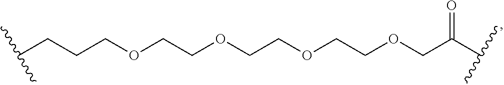 [SMILES: *CCCOCCOCCOCCOCC(*)=O] | (385) |
| 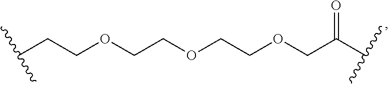 [SMILES: *CCOCCOCCOCC(*)=O] | (386) |
| 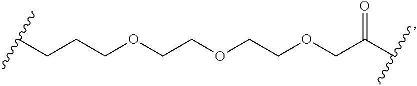 [SMILES: *CCCOCCOCCOCC(*)=O] | (387) |
| 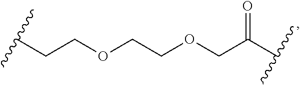 [SMILES: *CCOCCOCC(*)=O] | (388) |
| 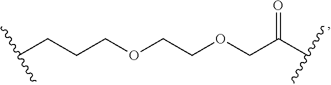 [SMILES: *CCCOCCOCC(*)=O] | (389) |
| 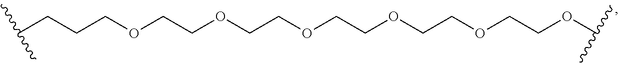 [SMILES: *CCCOCCOCCOCCOCCOCCO*] | (390) |
| 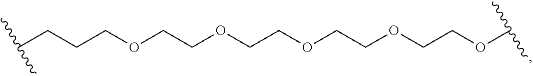 [SMILES: *CCCOCCOCCOCCOCCO*] | (391) |
| 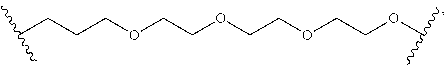 [SMILES: *CCCOCCOCCOCCO*] | (392) |
| 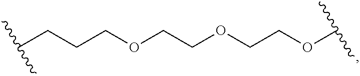 [SMILES: *CCCOCCOCCO*] | (393) |
| 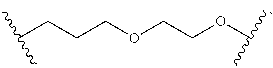 [SMILES: *CCCOCCO*] | (394) |
| 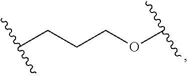 [SMILES: *CCCO*] | (395) |
| 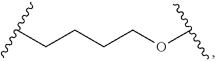 [SMILES: *CCCCO*] | (396) |
| 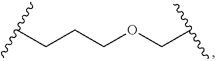 [SMILES: *CCCOC*] | (397) |
| 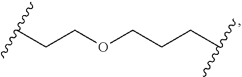 [SMILES: *CCCOCC*] | (398) |
| 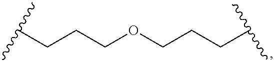 [SMILES: *CCCOCCC*] | (399) |
| 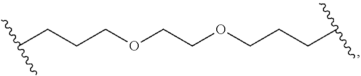 [SMILES: *CCCOCCOCCC*] | (400) |
| 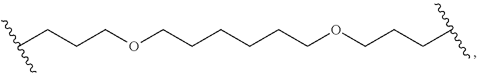 [SMILES: *CCCOCCCCCCOCCC*] | (401) |
| 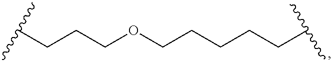 [SMILES: *CCCCCOCCC*] | (402) |
| 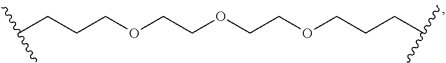 [SMILES: *CCCOCCOCCOCCC*] | (403) |
| 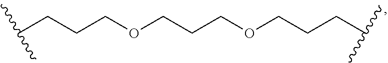 [SMILES: *CCCOCCCOCCC*] | (404) |
| 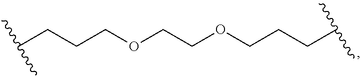 [SMILES: *CCCOCCOCCC*] | (405) |
| 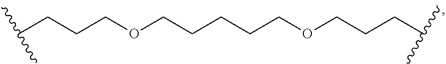 [SMILES: *CCCOCCCCCOCCC*] | (406) |
| 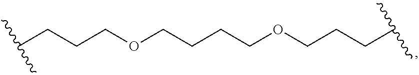 [SMILES: *CCCOCCCCOCCC*] | (407) |
| 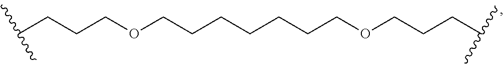 [SMILES: *CCCOCCCCCCCOCCC*] | (408) |
| 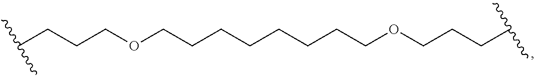 [SMILES: *CCCOCCCCCCCCOCCC*] | (409) |
| 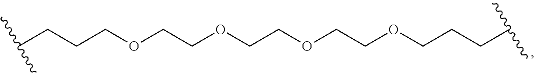 [SMILES: *CCCOCCOCCOCCOCCC*] | (410) |
| 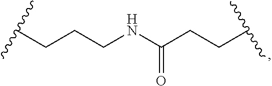 [SMILES: *CCCNC(=O)CC*] | (411) |
| 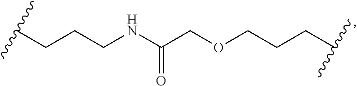 [SMILES: *CCCNC(=O)COCCC*] | (412) |
| 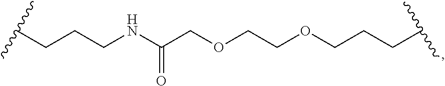 [SMILES: *CCCNC(=O)COCCOCCC*] | (413) |
| 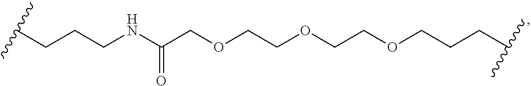 [SMILES: *CCCNC(=O)COCCOCCOCCC*] | (414) |
| 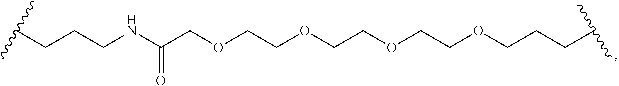 [SMILES: *CCCNC(=O)COCCOCCOCCOCCC*] | (415) |
| 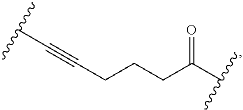 [SMILES: *C#CCCCC(*)=O] | (416) |
| 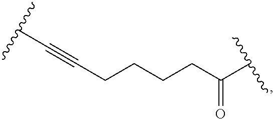 [SMILES: *C#CCCCCC(*)=O] | (417) |
| 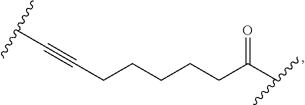 [SMILES: *C#CCCCCCC(*)=O] | (418) |
| 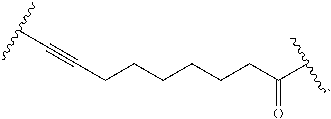 [SMILES: *C#CCCCCCCC(*)=O] | (419) |
| 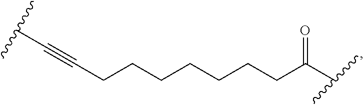 [SMILES: *C#CCCCCCCCC(*)=O] | (420) |
| 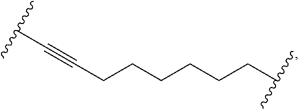 [SMILES: *C#CCCCCCC*] | (421) |
| 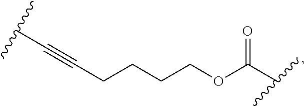 [SMILES: *C#CCCCCOC(*)=O] | (422) |
| 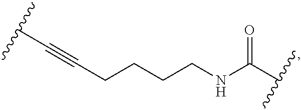 [SMILES: *C#CCCCCNC(*)=O] | (423) |
| 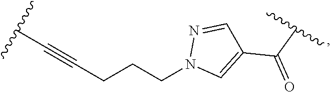 [SMILES: *C#CCCCn1cc(C(*)=O)cn1] | (424) |
| 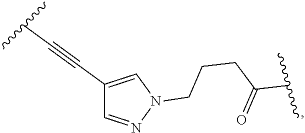 [SMILES: *C#Cc1cnn(CCCC(*)=O)c1] | (425) |
| 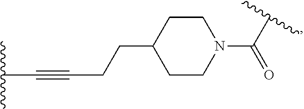 [SMILES: *C#CCCC1CCN(C(*)=O)CC1] | (426) |
| 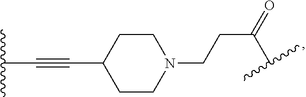 [SMILES: *C#CC1CCN(CCC(*)=O)CC1] | (427) |
| 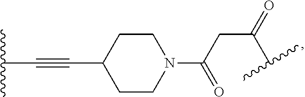 [SMILES: *C#CC1CCN(C(=O)CC(*)=O)CC1] | (428) |
| 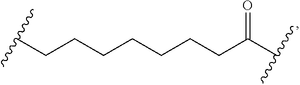 [SMILES: *CCCCCCCC(*)=O] | (429) |
| 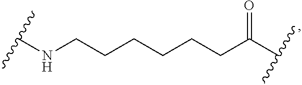 [SMILES: *NCCCCCCC(*)=O] | (430) |
| 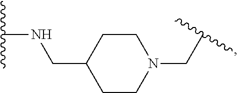 [SMILES: *CN1CCC(CN*)CC1] | (431) |
| 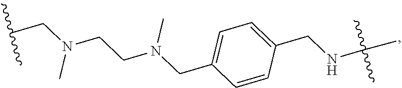 [SMILES: *CN(C)CCN(C)Cc1ccc(CN*)cc1] | (432) |
| 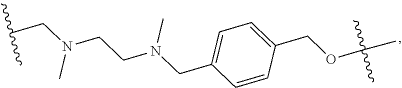 [SMILES: *CN(C)CCN(C)Cc1ccc(CO*)cc1] | (433) |
| 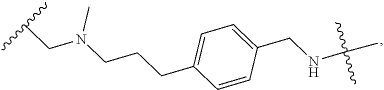 [SMILES: *CN(C)CCCc1ccc(CN*)cc1] | (434) |
| 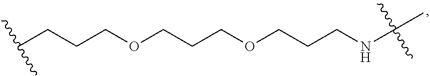 [SMILES: *CCCOCCCOCCCN*] | (435) |
| 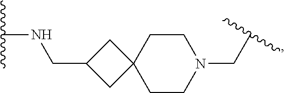 [SMILES: *CN1CCC2(CC1)CC(CN*)C2] | (436) |
| 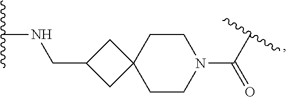 [SMILES: *NCC1CC2(CCN(C(*)=O)CC2)C1] | (437) |
| 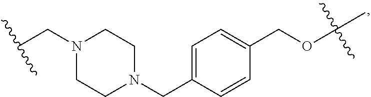 [SMILES: *CN1CCN(Cc2ccc(CO*)cc2)CC1] | (438) |
| 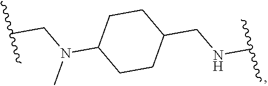 [SMILES: *CN(C)C1CCC(CN*)CC1] | (438) |
| 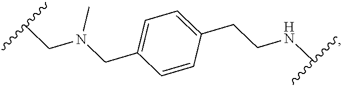 [SMILES: *CN(C)Cc1ccc(CCN*)cc1] | (439) |
| 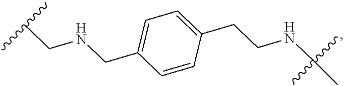 [SMILES: *CNCc1ccc(CCN*)cc1] | (440) |
| 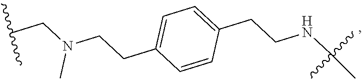 [SMILES: *CN(C)CCc1ccc(CCN*)cc1] | (441) |
| 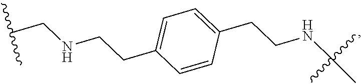 [SMILES: *CNCCc1ccc(CCN*)cc1] | (442) |
| 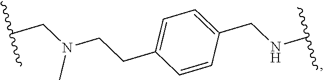 [SMILES: *CN(C)CCc1ccc(CN*)cc1] | (443) |
| 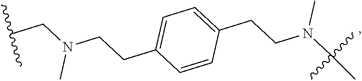 [SMILES: *CN(C)CCc1ccc(CCN(*)C)cc1] | (444) |
| 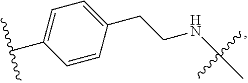 [SMILES: *NCCc1ccc(*)cc1] | (445) |
| 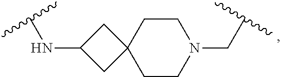 [SMILES: *CN1CCC2(CC1)CC(N*)C2] | (446) |
| 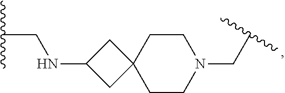 [SMILES: *CNC1CC2(CCN(C*)CC2)C1] | (447) |
| 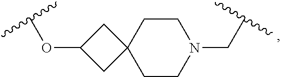 [SMILES: *CN1CCC2(CC1)CC(O*)C2] | (448) |
| 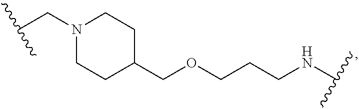 [SMILES: *CN1CCC(COCCCN*)CC1] | (449) |
| 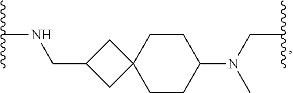 [SMILES: *CN(C)C1CCC2(CC1)CC(CN*)C2] | (450) |
| 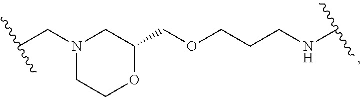 [SMILES: *CN1CCO[C@@H](COCCCN*)C1] | (451) |
| 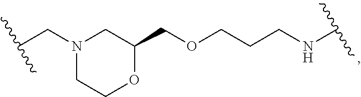 [SMILES: *CN1CCO[C@H](COCCCN*)C1] | (452) |
| 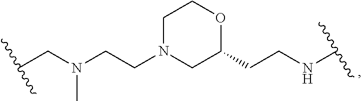 [SMILES: *CN(C)CCN1CCO[C@H](CCN*)C1] | (453) |
| 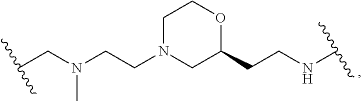 [SMILES: *CN(C)CCN1CCO[C@@H](CCN*)C1] | (454) |
| 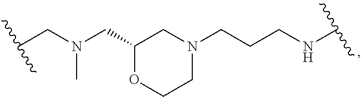 [SMILES: *CN(C)C[C@@H]1CN(CCCN*)CCO1] | (455) |
| 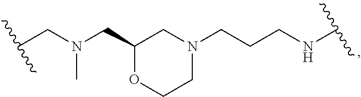 [SMILES: *CN(C)C[C@H]1CN(CCCN*)CCO1] | (456) |
| 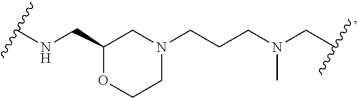 [SMILES: *CN(C)CCCN1CCO[C@@H](CN*)C1] | (457) |
| 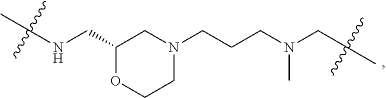 [SMILES: *CN(C)CCCN1CCO[C@H](CN*)C1] | (458) |
| 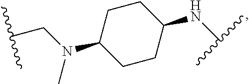 [SMILES: *CN(C)[C@H]1CC[C@@H](N*)CC1] | (459) |
| 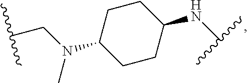 [SMILES: *CN(C)[C@H]1CC[C@H](N*)CC1] | (460) |
| 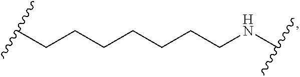 [SMILES: *CCCCCCCN*] | (461) |
| 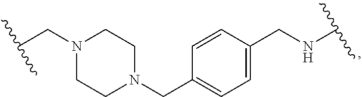 [SMILES: *CN1CCN(Cc2ccc(CN*)cc2)CC1] | (462) |
| 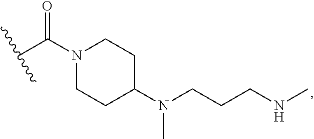 [SMILES: *C(=O)N1CCC(N(C)CCCNC)CC1] | (463) |
| 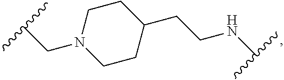 [SMILES: *CN1CCC(CCN*)CC1] | (464) |
| 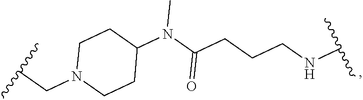 [SMILES: *CN1CCC(N(C)C(=O)CCCN*)CC1] | (465) |
| 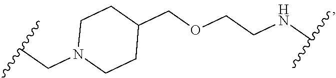 [SMILES: *CN1CCC(COCCN*)CC1] | (466) |
| 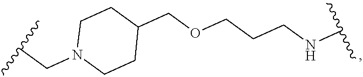 [SMILES: *CN1CCC(COCCCN*)CC1] | (467) |
| 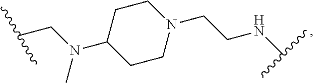 [SMILES: *CN(C)C1CCN(CCN*)CC1] | (468) |
| 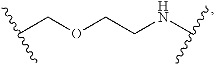 [SMILES: *COCCN*] | (469) |
| 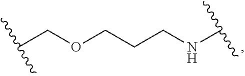 [SMILES: *COCCCN*] | (470) |
| 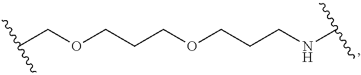 [SMILES: *COCCCOCCCN*] | (471) |
| 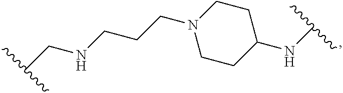 [SMILES: *CNCCCN1CCC(N*)CC1] | (472) |
| 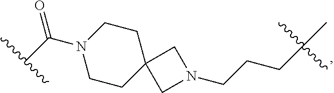 [SMILES: *CCCN1CC2(CCN(C(*)=O)CC2)C1] | (473) |
| 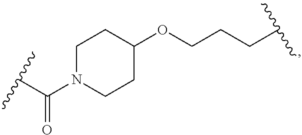 [SMILES: *CCCOC1CCN(C(*)=O)CC1] | (474) |
| 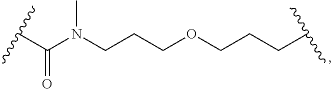 [SMILES: *CCCOCCCN(C)C(*)=O] | (475) |
| 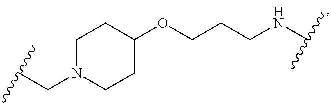 [SMILES: *CN1CCC(OCCCN*)CC1] | (475) |
| 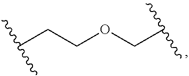 [SMILES: *CCOC*] | (476) |
| 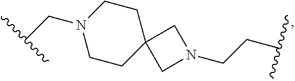 [SMILES: *CCN1CC2(CCN(C*)CC2)C1] | (477) |
| 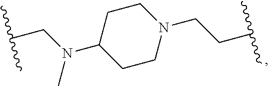 [SMILES: *CCN1CCC(N(C)C*)CC1] | (478) |
| 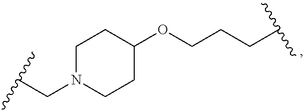 [SMILES: *CCCOC1CCN(C*)CC1] | (479) |
| 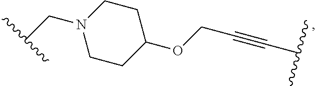 [SMILES: *C#CCOC1CCN(C*)CC1] | (480) |
| 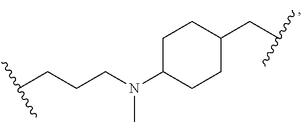 [SMILES: *CCCN(C)C1CCC(C*)CC1] | (481) |
| 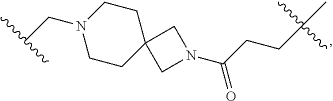 [SMILES: *CCC(=O)N1CC2(CCN(C*)CC2)C1] | (482) |
| 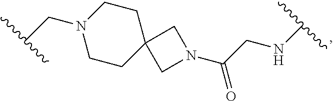 [SMILES: *CN1CCC2(CC1)CN(C(=O)CN*)C2] | (483) |
| 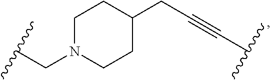 [SMILES: *C#CCC1CCN(C*)CC1] | (484) |
| 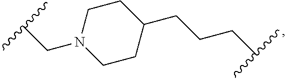 [SMILES: *CCCC1CCN(C*)CC1] | (485) |
| 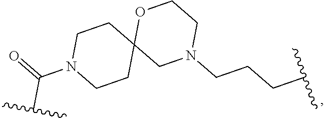 [SMILES: *CCCN1CCOC2(CCN(C(*)=O)CC2)C1] | (486) |
| 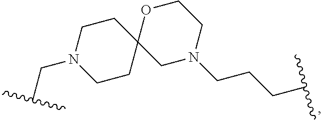 [SMILES: *CCCN1CCOC2(CCN(C*)CC2)C1] | (487) |
| 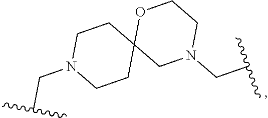 [SMILES: *CN1CCC2(CC1)CN(C*)CCO2] | (488) |
| 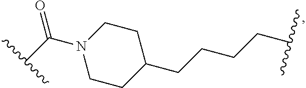 [SMILES: *CCCCC1CCN(C(*)=O)CC1] | (489) |
| 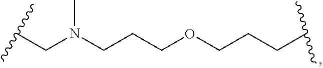 [SMILES: *CCCOCCCN(C)C*] | (490) |
| 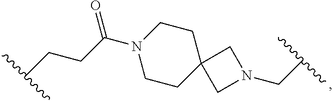 [SMILES: *CCC(=O)N1CCC2(CC1)CN(C*)C2] | (491) |
| 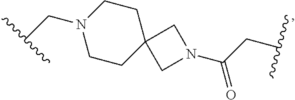 [SMILES: *CC(=O)N1CC2(CCN(C*)CC2)C1] | (492) |
| 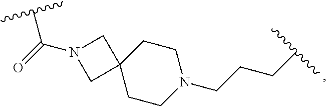 [SMILES: *CCCN1CCC2(CC1)CN(C(*)=O)C2] | (493) |
| 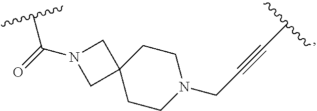 [SMILES: *C#CCN1CCC2(CC1)CN(C(*)=O)C2] | (494) |
| 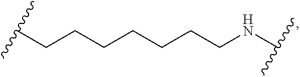 [SMILES: *CCCCCCCN*] | (495) |
| 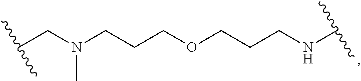 [SMILES: *CN(C)CCCOCCCN*] | (496) |
| 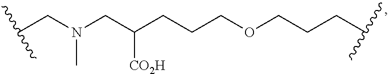 [SMILES: *CCCOCCCC(CN(C)C*)C(=O)O] | (497) |
| 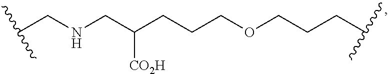 [SMILES: *CCCOCCCC(CNC*)C(=O)O] | (498) |
| 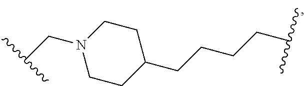 [SMILES: *CCCCC1CCN(C*)CC1] | (499) |
| 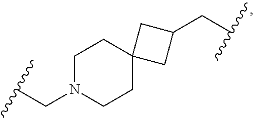 [SMILES: *CC1CC2(CCN(C*)CC2)C1] | (500) |
| 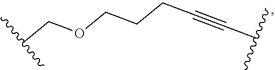 [SMILES: *C#CCCCOC*] | (501) |
| 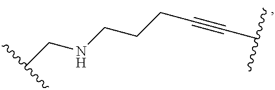 [SMILES: *C#CCCCNC*] | (502) |
| 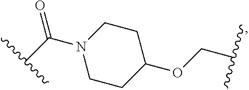 [SMILES: *COC1CCN(C(*)=O)CC1] | (503) |
| 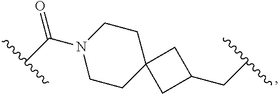 [SMILES: *CC1CC2(CCN(C(*)=O)CC2)C1] | (504) |
| 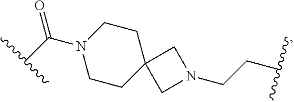 [SMILES: *CCN1CC2(CCN(C(*)=O)CC2)C1] | (505) |
| 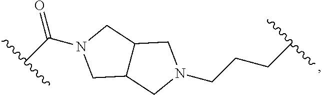 [SMILES: *CCCN1CC2CN(C(*)=O)CC2C1] | (506) |
| 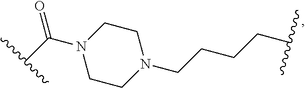 [SMILES: *CCCCN1CCN(C(*)=O)CC1] | (507) |
| 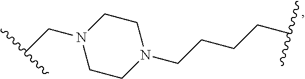 [SMILES: *CCCCN1CCN(C*)CC1] | (508) |
| 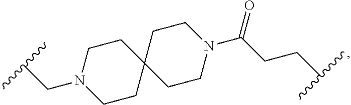 [SMILES: *CCC(=O)N1CCC2(CCN(C*)CC2)CC1] | (509) |
| 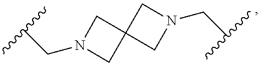 [SMILES: *CN1CC2(C1)CN(C*)C2] | (510) |
| 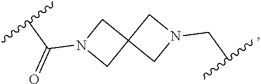 [SMILES: *CN1CC2(C1)CN(C(*)=O)C2] | (511) |
| 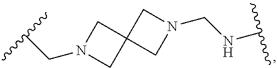 [SMILES: *CN1CC2(C1)CN(CN*)C2] | (512) |
| 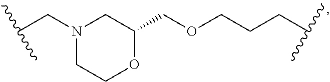 [SMILES: *CCCOC[C@H]1CN(C*)CCO1] | (513) |
| 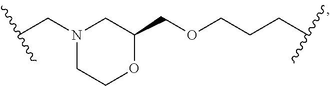 [SMILES: *CCCOC[C@@H]1CN(C*)CCO1] | (514) |
| 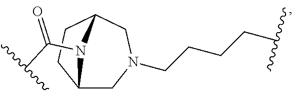 [SMILES: *CCCCN1C[C@H]2CC[C@@H](C1)N2C(*)=O] | (515) |
| 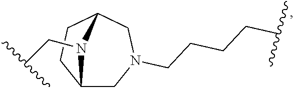 [SMILES: *CCCCN1C[C@H]2CC[C@@H](C1)N2C*] | (516) |
| 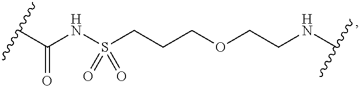 [SMILES: *NCCOCCCS(=O)(=O)NC(*)=O] | (517) |
| 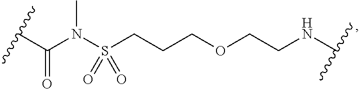 [SMILES: *NCCOCCCS(=O)(=O)N(C)C(*)=O] | (518) |
| 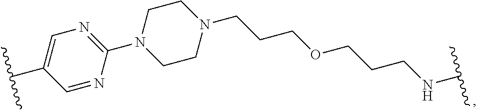 [SMILES: *NCCCOCCCN1CCN(c2ncc(*)cn2)CC1] | (519) |
| 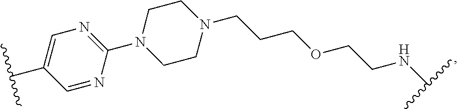 [SMILES: *NCCOCCCN1CCN(c2ncc(*)cn2)CC1] | (520) |
| 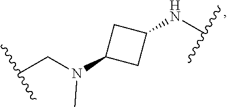 [SMILES: *CN(C)[C@H]1C[C@H](N*)C1] | (521) |
| 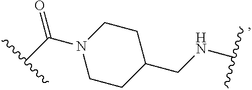 [SMILES: *NCC1CCN(C(*)=O)CC1] | (522) |
| 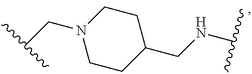 [SMILES: *CN1CCC(CN*)CC1] | (523) |
| 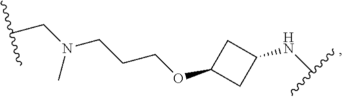 [SMILES: *CN(C)CCCO[C@H]1C[C@H](N*)C1] | (524) |
| 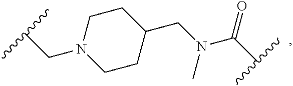 [SMILES: *CN1CCC(CN(C)C(*)=O)CC1] | (525) |
| 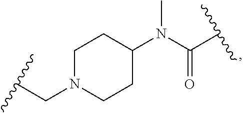 [SMILES: *CN1CCC(N(C)C(*)=O)CC1] | (526) |
| 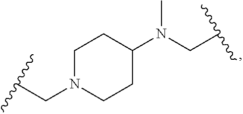 [SMILES: *CN1CCC(N(C)C*)CC1] | (527) |
| 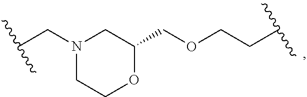 [SMILES: *CCOC[C@H]1CN(C*)CCO1] | (528) |
| 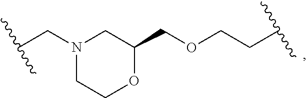 [SMILES: *CCOC[C@@H]1CN(C*)CCO1] | (529) |
| 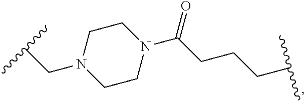 [SMILES: *CCCC(=O)N1CCN(C*)CC1] | (530) |
| 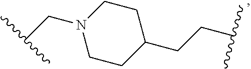 [SMILES: *CCC1CCN(C*)CC1] | (531) |
| 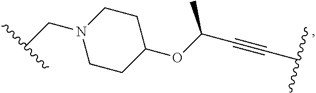 [SMILES: *C#C[C@H](C)OC1CCN(C*)CC1] | (532) |
| 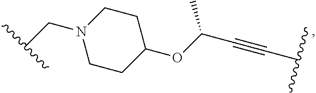 [SMILES: *C#C[C@@H](C)OC1CCN(C*)CC1] | (533) |
| 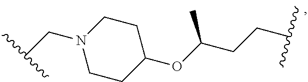 [SMILES: *CC[C@H](C)OC1CCN(C*)CC1] | (534) |
| 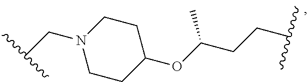 [SMILES: *CC[C@@H](C)OC1CCN(C*)CC1] | (535) |
| 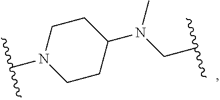 [SMILES: *CN(C)C1CCN(*)CC1] | (536) |
| 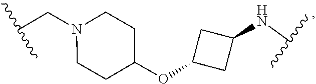 [SMILES: *CN1CCC(O[C@H]2C[C@H](N*)C2)CC1] | (537) |
| 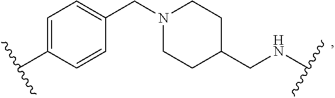 [SMILES: *NCC1CCN(Cc2ccc(*)cc2)CC1] | (538) |
| 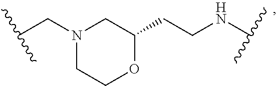 [SMILES: *CN1CCO[C@@H](CCN*)C1] | (539) |
| 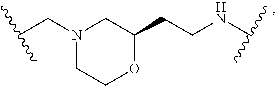 [SMILES: *CN1CCO[C@H](CCN*)C1] | (540) |
| 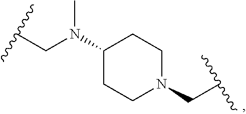 | (541) |
| 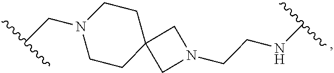 [SMILES: *CN1CCC2(CC1)CN(CCN*)C2] | (542) |
| 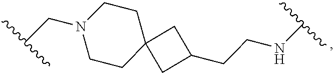 [SMILES: *CN1CCC2(CC1)CC(CCN*)C2] | (543) |
| 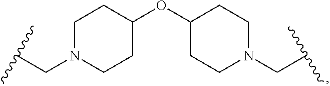 [SMILES: *CN1CCC(OC2CCN(C*)CC2)CC1] | (544) |
| 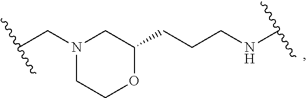 [SMILES: *CN1CCO[C@@H](CCCN*)C1] | (545) |
| 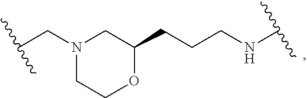 [SMILES: *CN1CCO[C@H](CCCN*)C1] | (546) |
| 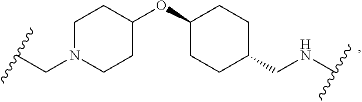 [SMILES: *CN1CCC(O[C@H]2CC[C@H](CN*)CC2)CC1] | (547) |
| 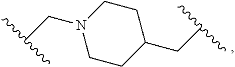 [SMILES: *CC1CCN(C*)CC1] | (548) |
| 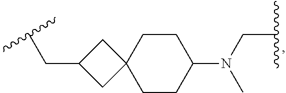 [SMILES: *CC1CC2(CCC(N(C)C*)CC2)C1] | (549) |
| 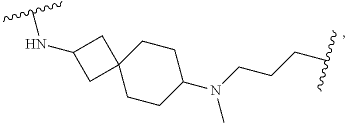 [SMILES: *CCCN(C)C1CCC2(CC1)CC(N*)C2] | (550) |
| 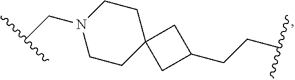 [SMILES: *CCC1CC2(CCN(C*)CC2)C1] | (551) |
| 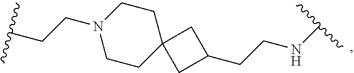 [SMILES: *CCN1CCC2(CC1)CC(CCN*)C2] | (552) |
| 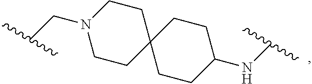 [SMILES: *CN1CCC2(CCC(N*)CC2)CC1] | (553) |
| 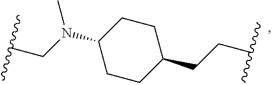 [SMILES: *CC[C@H]1CC[C@H](N(C)C*)CC1] | (554) |
| 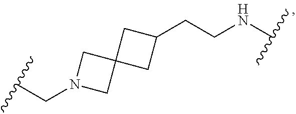 [SMILES: *CN1CC2(CC(CCN*)C2)C1] | (555) |
| 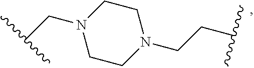 [SMILES: *CCN1CCN(C*)CC1] | (556) |
| 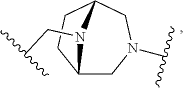 [SMILES: *CN1[C@@H]2CC[C@H]1CN(*)C2] | (557) |
| 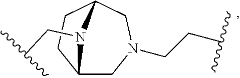 [SMILES: *CCN1C[C@H]2CC[C@@H](C1)N2C*] | (558) |
| 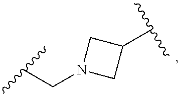 [SMILES: *CN1CC(*)C1] | (559) |
| 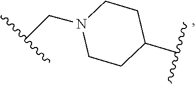 [SMILES: *CN1CCC(*)CC1] | (560) |
| 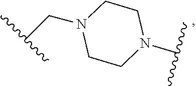 [SMILES: *CN1CCN(*)CC1] | (561) |
| 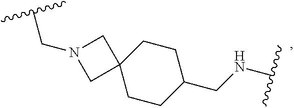 [SMILES: *CN1CC2(CCC(CN*)CC2)C1] | (562) |
| 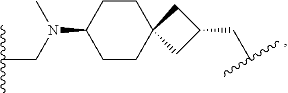 | (563) |
| 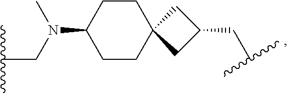 | (564) |
| 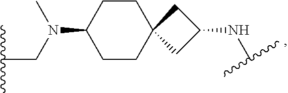 | (565) |
| 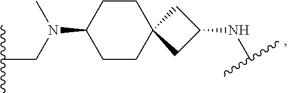 | (566) |
| 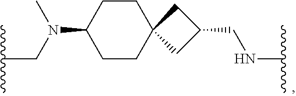 | (567) |
| 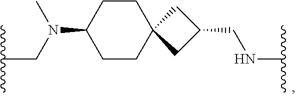 | (568) |
| 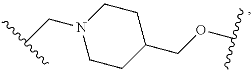 [SMILES: *CN1CCC(CO*)CC1] | (569) |
| 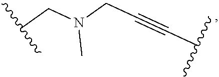 [SMILES: *C#CCN(C)C*] | (570) |
| 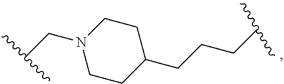 [SMILES: *CCCC1CCN(C*)CC1] | (571) |
| 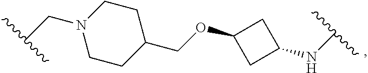 [SMILES: *CN1CCC(CO[C@H]2C[C@H](N*)C2)CC1] | (572) |
| 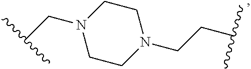 [SMILES: *CCN1CCN(C*)CC1] | (573) |
| 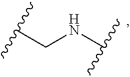 [SMILES: *CN*] | (574) |
| 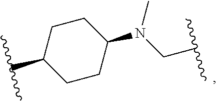 [SMILES: *CN(C)[C@H]1CC[C@@H](*)CC1] | (575) |
| 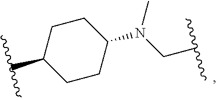 [SMILES: *CN(C)[C@H]1CC[C@H](*)CC1] | (576) |
| 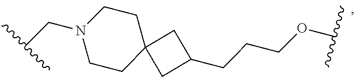 [SMILES: *CN1CCC2(CC1)CC(CCCO*)C2] | (577) |
| 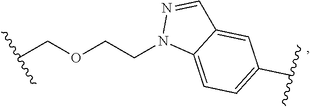 [SMILES: *COCCn1ncc2cc(*)ccc21] | (578) |
| 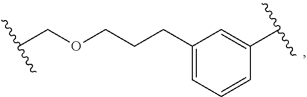 [SMILES: *COCCCc1cccc(*)c1] | (579) |
| 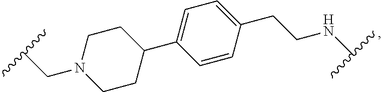 [SMILES: *CN1CCC(c2ccc(CCN*)cc2)CC1] | (580) |
| 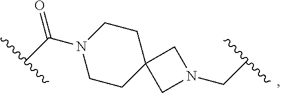 [SMILES: *CN1CC2(CCN(C(*)=O)CC2)C1] | (581) |
| 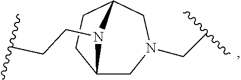 [SMILES: *CCN1[C@@H]2CC[C@H]1CN(C*)C2] | (582) |
| 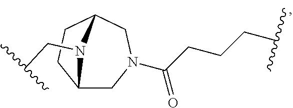 [SMILES: *CCCC(=O)N1C[C@H]2CC[C@@H](C1)N2C*] | (583) |
| 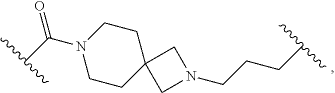 [SMILES: *CCCN1CC2(CCN(C(*)=O)CC2)C1] | (584) |
| 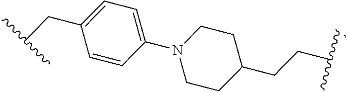 [SMILES: *CCC1CCN(c2ccc(C*)cc2)CC1] | (585) |
| 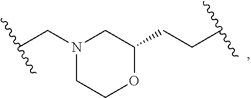 [SMILES: *CC[C@H]1CN(C*)CCO1] | (586) |
| 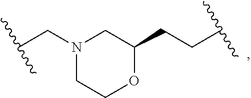 [SMILES: *CC[C@@H]1CN(C*)CCO1] | (587) |
| 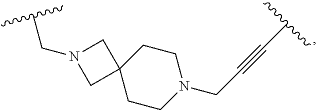 [SMILES: *C#CCN1CCC2(CC1)CN(C*)C2] | (588) |
| 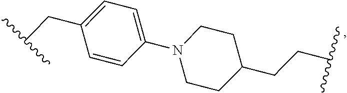 [SMILES: *CCC1CCN(c2ccc(C*)cc2)CC1] | (589) |
| 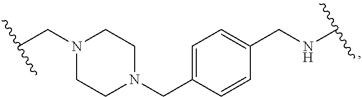 [SMILES: *CN1CCN(Cc2ccc(CN*)cc2)CC1] | (590) |
| 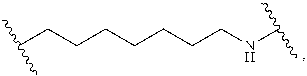 [SMILES: *CCCCCCCN*] | (591) |
| 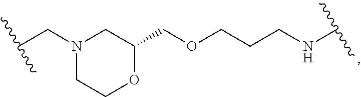 [SMILES: *CN1CCO[C@@H](COCCCN*)C1] | (592) |
| 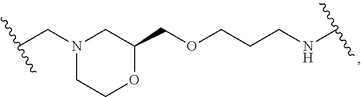 [SMILES: *CN1CCO[C@H](COCCCN*)C1] | (593) |
| 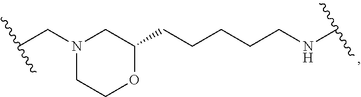 [SMILES: *CN1CCO[C@@H](CCCCCN*)C1] | (594) |
| 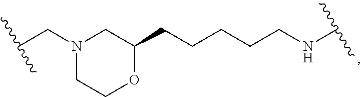 [SMILES: *CN1CCO[C@H](CCCCCN*)C1] | (595) |
| 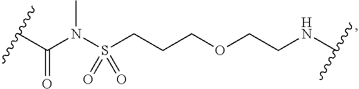 [SMILES: *NCCOCCCS(=O)(=O)N(C)C(*)=O] | (596) |
| 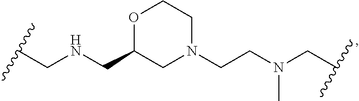 [SMILES: *CNC[C@@H]1CN(CCN(C)C*)CCO1] | (597) |
| 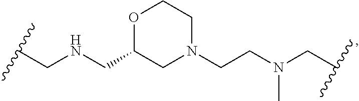 [SMILES: *CNC[C@H]1CN(CCN(C)C*)CCO1] | (598) |
| 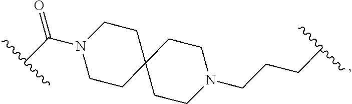 [SMILES: *CCCN1CCC2(CC1)CCN(C(*)=O)CC2] | (599) |
| 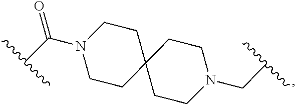 [SMILES: *CN1CCC2(CC1)CCN(C(*)=O)CC2] | (600) |
| 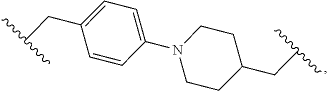 [SMILES: *Cc1ccc(N2CCC(C*)CC2)cc1] | (601) |
| 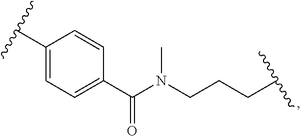 [SMILES: *CCCN(C)C(=O)c1ccc(*)cc1] | (602) |
| 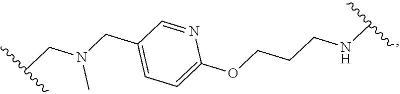 [SMILES: *CN(C)Cc1ccc(OCCCN*)nc1] | (603) |
| 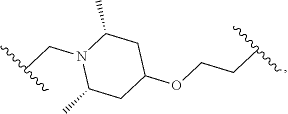 [SMILES: *CCOC1C[C@@H](C)N(C*)[C@@H](C)C1] | (604) |
| 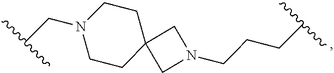 [SMILES: *CCCN1CC2(CCN(C*)CC2)C1] | (605) |
| 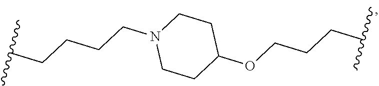 [SMILES: *CCCCN1CCC(OCCC*)CC1] | (606) |
| 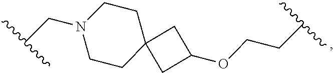 [SMILES: *CCOC1CC2(CCN(C*)CC2)C1] | (607) |
| 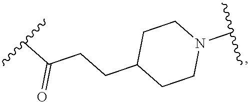 [SMILES: *C(=O)CCC1CCN(*)CC1] | (608) |
| 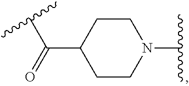 [SMILES: *C(=O)C1CCN(*)CC1] | (609) |
| 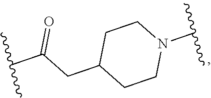 [SMILES: *C(=O)CC1CCN(*)CC1] | (610) |
| 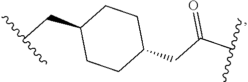 [SMILES: *C[C@H]1CC[C@H](CC(*)=O)CC1] | (611) |
| 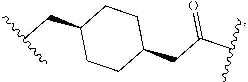 [SMILES: *C[C@H]1CC[C@@H](CC(*)=O)CC1] | (612) |
| 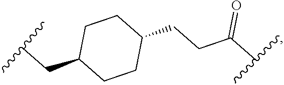 [SMILES: *C[C@H]1CC[C@H](CCC(*)=O)CC1] | (613) |
| 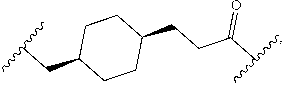 [SMILES: *C[C@H]1CC[C@@H](CCC(*)=O)CC1] | (614) |
| 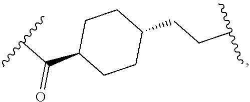 [SMILES: *CC[C@H]1CC[C@H](C(*)=O)CC1] | (615) |
| 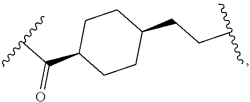 [SMILES: *CC[C@H]1CC[C@@H](C(*)=O)CC1] | (616) |
| 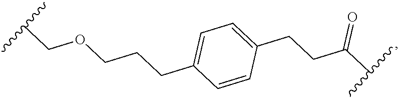 [SMILES: *COCCCc1ccc(CCC(*)=O)cc1] | (617) |
| 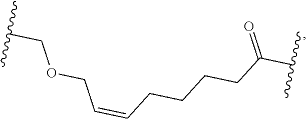 [SMILES: *COC/C=C\CCCCC(*)=O] | (618) |
| 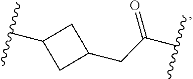 [SMILES: *C(=O)CC1CC(*)C1] | (619) |
| 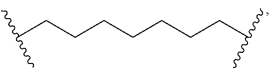 [SMILES: *CCCCCCC*] | (620) |
| 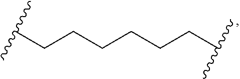 [SMILES: *CCCCCC*] | (621) |
| 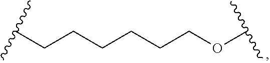 [SMILES: *CCCCCCO*] | (622) |
| 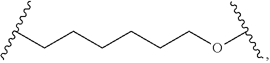 [SMILES: *CCCCCCO*] | (623) |
| 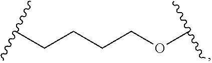 [SMILES: *CCCCO*] | (624) |
| 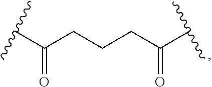 [SMILES: *C(=O)CCCC(*)=O] | (625) |
| 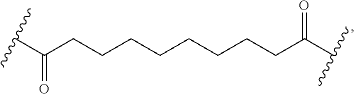 [SMILES: *C(=O)CCCCCCCCC(*)=O] | (626) |
| 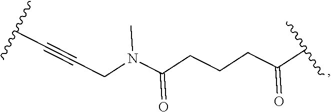 [SMILES: *C#CCN(C)C(=O)CCCC(*)=O] | (627) |
| 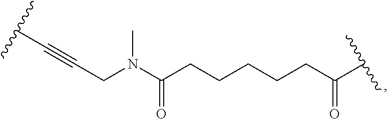 [SMILES: *C#CCN(C)C(=O)CCCCCC(*)=O] | (628) |
| 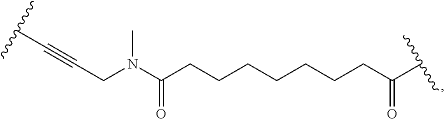 [SMILES: *C#CCN(C)C(=O)CCCCCCCC(*)=O] | (629) |
| 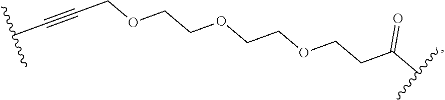 [SMILES: *C#CCOCCOCCOCCC(*)=O] | (630) |
| 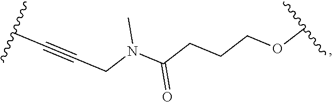 [SMILES: *C#CCN(C)C(=O)CCCO*] | (631) |
| 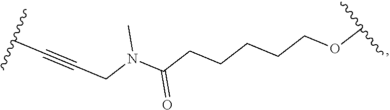 [SMILES: *C#CCN(C)C(=O)CCCCCO*] | (632) |
| 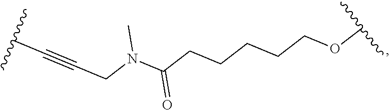 [SMILES: *C#CCN(C)C(=O)CCCCCO*] | (633) |
| 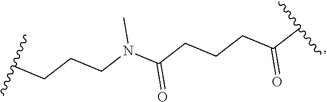 [SMILES: *CCCN(C)C(=O)CCCC(*)=O] | (634) |
| 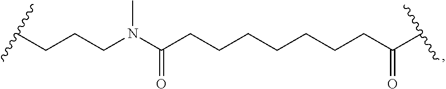 [SMILES: *CCCN(C)C(=O)CCCCCCCC(*)=O] | (635) |
| 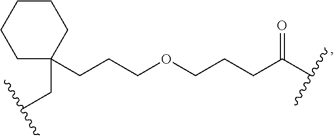 [SMILES: *CC1(CCCOCCCC(*)=O)CCCCC1] | (636) |
| 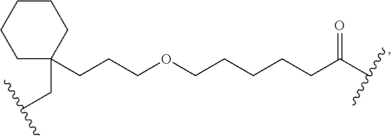 [SMILES: *CC1(CCCOCCCCCC(*)=O)CCCCC1] | (637) |
| 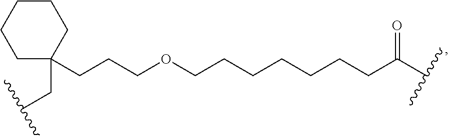 [SMILES: *CC1(CCCOCCCCCCCC(*)=O)CCCCC1] | (638) |
| 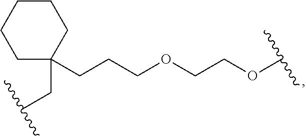 [SMILES: *CC1(CCCOCCO*)CCCCC1] | (639) |
| 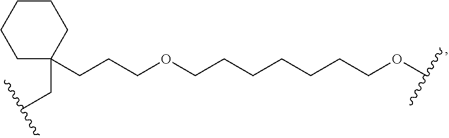 [SMILES: *CC1(CCCOCCCCCCCO*)CCCCC1] | (640) |
| 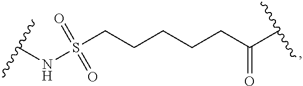 [SMILES: *NS(=O)(=O)CCCCCC(*)=O] | (641) |
| 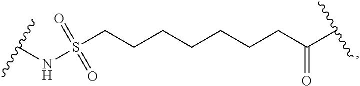 [SMILES: *NS(=O)(=O)CCCCCCCC(*)=O] | (642) |
| 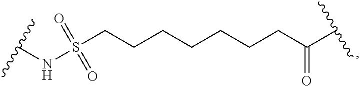 [SMILES: *NS(=O)(=O)CCCCCCCC(*)=O] | (643) |
| 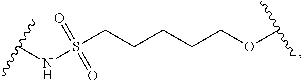 [SMILES: *NS(=O)(=O)CCCCCO*] | (644) |
| 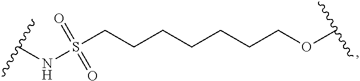 [SMILES: *NS(=O)(=O)CCCCCCCO*] | (645) |
| 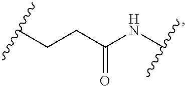 [SMILES: *CCC(=O)N*] | (646) |
| 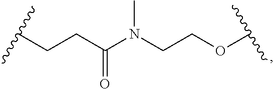 [SMILES: *CCC(=O)N(C)CCO*] | (647) |
| 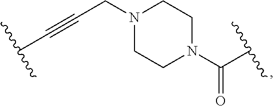 [SMILES: *C#CCN1CCN(C(*)=O)CC1] | (648) |
| 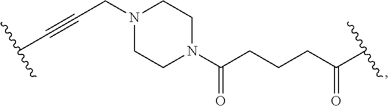 [SMILES: *C#CCN1CCN(C(=O)CCCC(*)=O)CC1] | (649) |
| 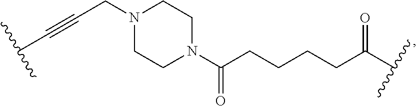 [SMILES: *C#CCN1CCN(C(=O)CCCCC(*)=O)CC1] | (650) |
| 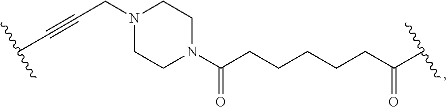 [SMILES: *C#CCN1CCN(C(=O)CCCCCC(*)=O)CC1] | (651) |
| 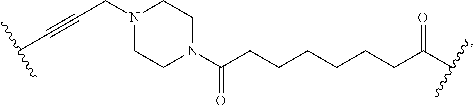 [SMILES: *C#CCN1CCN(C(=O)CCCCCCC(*)=O)CC1] | (652) |
| 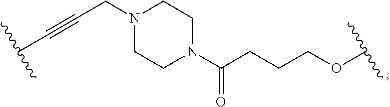 [SMILES: *C#CCN1CCN(C(=O)CCCO*)CC1] | (653) |
| 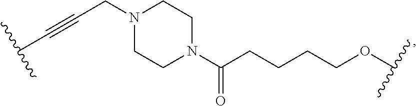 [SMILES: *C#CCN1CCN(C(=O)CCCCO*)CC1] | (654) |
| 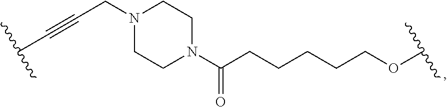 [SMILES: *C#CCN1CCN(C(=O)CCCCCO*)CC1] | (655) |
| 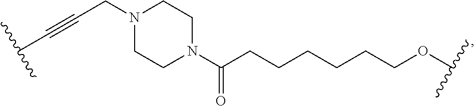 [SMILES: *C#CCN1CCN(C(=O)CCCCCCO*)CC1] | (656) |
| 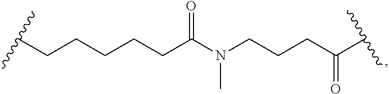 [SMILES: *CCCCCC(=O)N(C)CCCC(*)=O] | (657) |
| 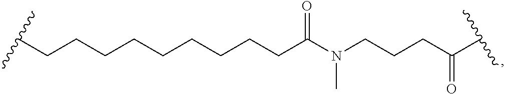 [SMILES: *CCCCCCCCCC(=O)N(C)CCCC(*)=O] | (658) |
| 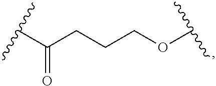 [SMILES: *OCCCC(*)=O] | (659) |
| 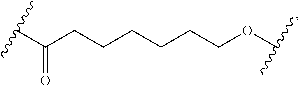 [SMILES: *OCCCCCCC(*)=O] | (660) |
| 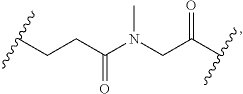 [SMILES: *CCC(=O)N(C)CC(*)=O] | (661) |
| 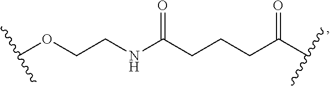 [SMILES: *OCCNC(=O)CCCC(*)=O] | (662) |
| 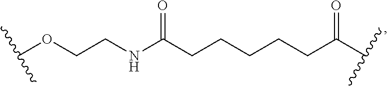 [SMILES: *OCCNC(=O)CCCCCC(*)=O] | (663) |
| 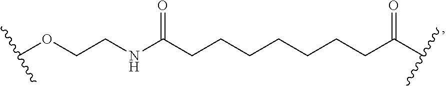 [SMILES: *OCCNC(=O)CCCCCCCC(*)=O] | (664) |
| 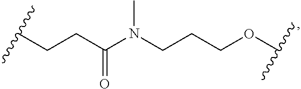 [SMILES: *CCC(=O)N(C)CCCO*] | (665) |
| 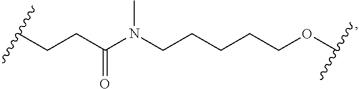 [SMILES: *CCC(=O)N(C)CCCCCO*] | (666) |
| 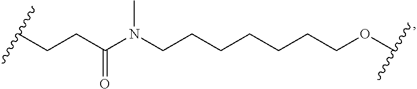 [SMILES: *CCC(=O)N(C)CCCCCCCO*] | (667) |
| 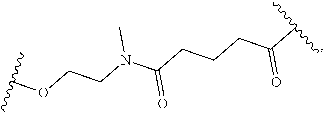 [SMILES: *OCCN(C)C(=O)CCCC(*)=O] | (668) |
| 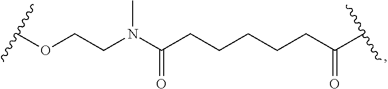 [SMILES: *OCCN(C)C(=O)CCCCCC(*)=O] | (669) |
| 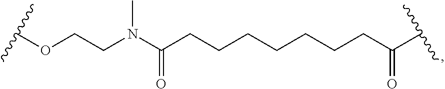 [SMILES: *OCCN(C)C(=O)CCCCCCCC(*)=O] | (670) |
| 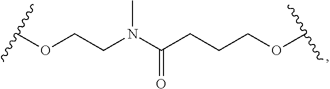 [SMILES: *OCCCC(=O)N(C)CCO*] | (671) |
| 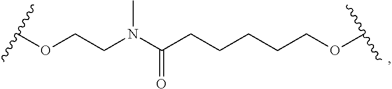 [SMILES: *OCCCCCC(=O)N(C)CCO*] | (672) |
| 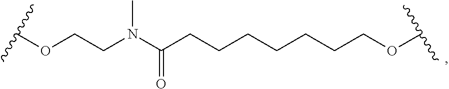 [SMILES: *OCCCCCCCC(=O)N(C)CCO*] | (673) |
| 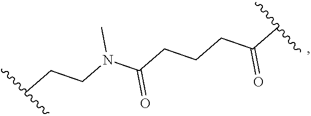 [SMILES: *CCN(C)C(=O)CCCC(*)=O] | (674) |
| 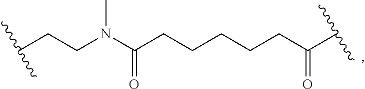 [SMILES: *CCN(C)C(=O)CCCCCC(*)=O] | (675) |
| 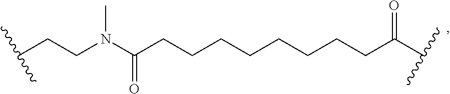 [SMILES: *CCN(C)C(=O)CCCCCCCCC(*)=O] | (676) |
| 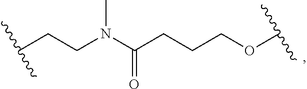 [SMILES: *CCN(C)C(=O)CCCO*] | (677) |
| 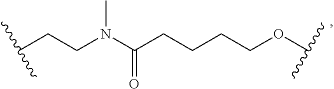 [SMILES: *CCN(C)C(=O)CCCCO*] | (678) |
| 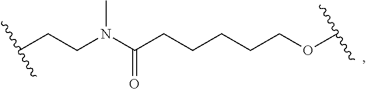 [SMILES: *CCN(C)C(=O)CCCCCO*] | (679) |
| 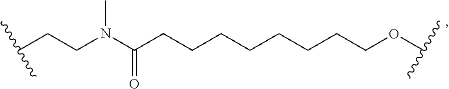 [SMILES: *CCN(C)C(=O)CCCCCCCCO*] | (680) |
| 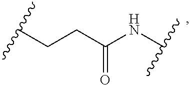 [SMILES: *CCC(=O)N*] | (681) |
| 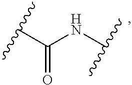 [SMILES: *NC(*)=O] | (682) |
| 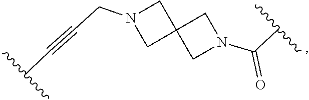 [SMILES: *C#CCN1CC2(C1)CN(C(*)=O)C2] | (683) |
| 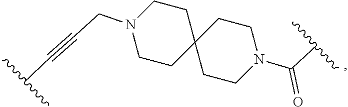 [SMILES: *C#CCN1CCC2(CC1)CCN(C(*)=O)CC2] | (684) |
| 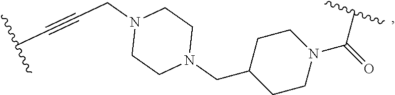 [SMILES: *C#CCN1CCN(CC2CCN(C(*)=O)CC2)CC1] | (685) |
| 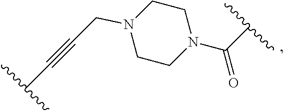 [SMILES: *C#CCN1CCN(C(*)=O)CC1] | (686) |
| 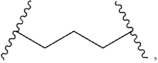 [SMILES: *CCC*] | (687) |
| 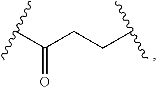 [SMILES: *CCC(*)=O] | (688) |
| 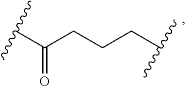 [SMILES: *CCCC(*)=O] | (689) |
| 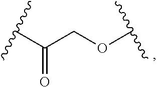 [SMILES: *OCC(*)=O] | (690) |
| 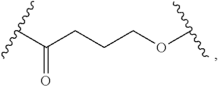 [SMILES: *OCCCC(*)=O] | (691) |
| 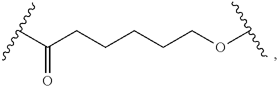 [SMILES: *OCCCCCC(*)=O] | (692) |
| 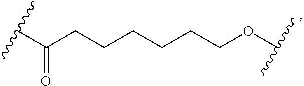 [SMILES: *OCCCCCCC(*)=O] | (693) |
| 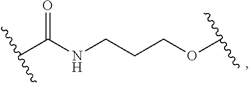 [SMILES: *OCCCNC(*)=O] | (694) |
| 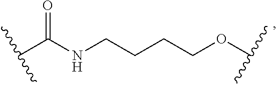 [SMILES: *OCCCCNC(*)=O] | (695) |
| 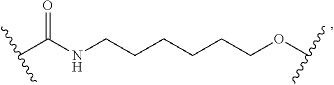 [SMILES: *OCCCCCCNC(*)=O] | (696) |
| 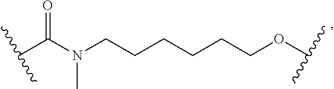 [SMILES: *OCCCCCCN(C)C(*)=O] | (697) |
| 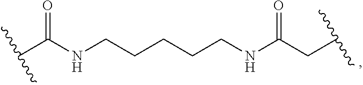 [SMILES: *CC(=O)NCCCCCNC(*)=O] | (698) |
| 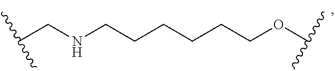 [SMILES: *CNCCCCCCO*] | (699) |
| 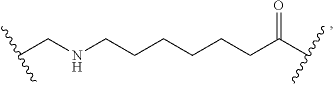 [SMILES: *CNCCCCCCC(*)=O] | (700) |
| 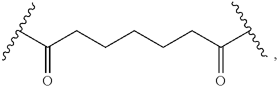 [SMILES: *C(=O)CCCCCC(*)=O] | (701) |
| 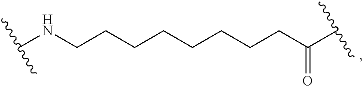 [SMILES: *NCCCCCCCCC(*)=O] | (702) |
| 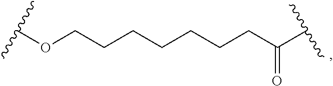 [SMILES: *OCCCCCCCC(*)=O] | (703) |
| 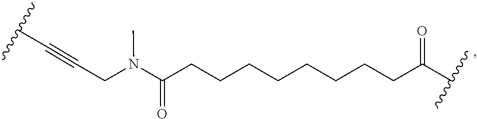 [SMILES: *C#CCN(C)C(=O)CCCCCCCCC(*)=O] | (704) |
| 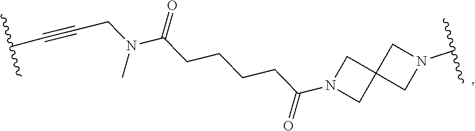 [SMILES: *C#CCN(C)C(=O)CCCCC(=O)N1CC2(CN(*)C2)C1] | (705) |
| 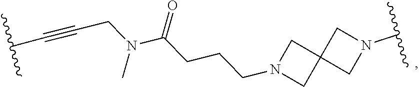 [SMILES: *C#CCN(C)C(=O)CCCN1CC2(CN(*)C2)C1] | (706) |
| 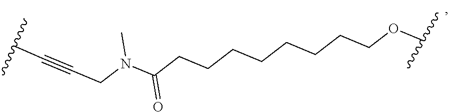 [SMILES: *C#CCN(C)C(=O)CCCCCCCCO*] | (707) |
| 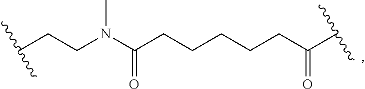 [SMILES: *CCN(C)C(=O)CCCCCC(*)=O] | (708) |
| 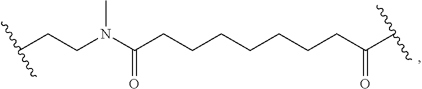 [SMILES: *CCN(C)C(=O)CCCCCCCC(*)=O] | (709) |
| 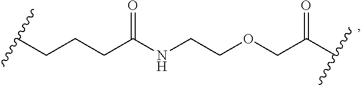 [SMILES: *CCCC(=O)NCCOCC(*)=O] | (710) |
| 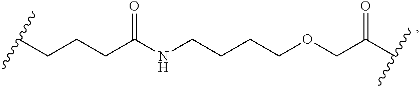 [SMILES: *CCCC(=O)NCCCCOCC(*)=O] | (711) |
| 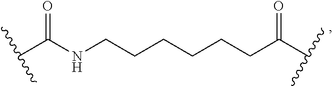 [SMILES: *C(=O)CCCCCCNC(*)=O] | (712) |
| 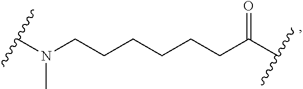 [SMILES: *C(=O)CCCCCCN(*)C] | (713) |
| 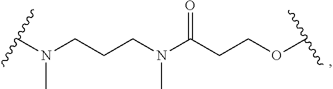 [SMILES: *OCCC(=O)N(C)CCCN(*)C] | (714) |
| 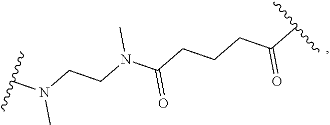 [SMILES: *C(=O)CCCC(=O)N(C)CCN(*)C] | (715) |
| 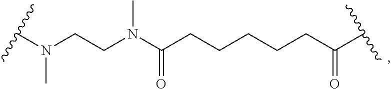 [SMILES: *C(=O)CCCCCC(=O)N(C)CCN(*)C] | (716) |
| 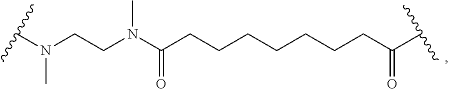 [SMILES: *C(=O)CCCCCCCC(=O)N(C)CCN(*)C] | (717) |
| 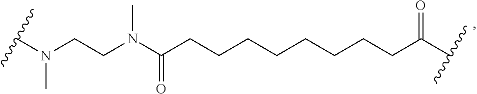 [SMILES: *C(=O)CCCCCCCCC(=O)N(C)CCN(*)C] | (718) |
| 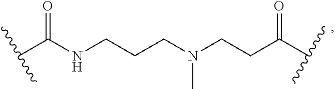 [SMILES: *C(=O)CCN(C)CCCNC(*)=O] | (719) |
| 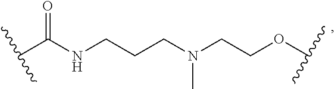 [SMILES: *OCCN(C)CCCNC(*)=O] | (720) |
| 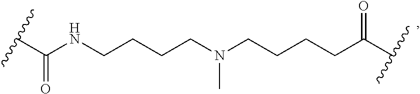 [SMILES: *C(=O)CCCCN(C)CCCCNC(*)=O] | (721) |
| —O—, | (722) |
| 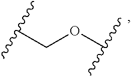 [SMILES: *CO*] | (723) |
| 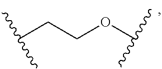 [SMILES: *CCO*] | (724) |
| 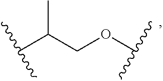 [SMILES: *OCC(*)C] | (725) |
| 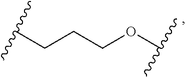 [SMILES: *CCCO*] | (726) |
| 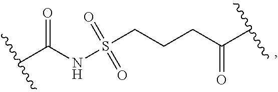 [SMILES: *C(=O)CCCS(=O)(=O)NC(*)=O] | (727) |
| 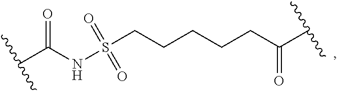 [SMILES: *C(=O)CCCCCS(=O)(=O)NC(*)=O] | (728) |
| 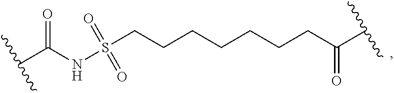 [SMILES: *C(=O)CCCCCCCS(=O)(=O)NC(*)=O] | (729) |
| 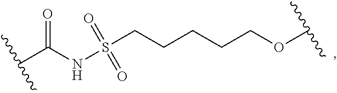 [SMILES: *OCCCCCS(=O)(=O)NC(*)=O] | (730) |
| 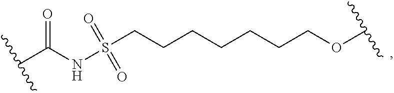 [SMILES: *OCCCCCCCS(=O)(=O)NC(*)=O] | (731) |
| 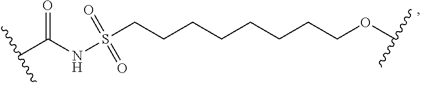 [SMILES: *OCCCCCCCCS(=O)(=O)NC(*)=O] | (732) |
| 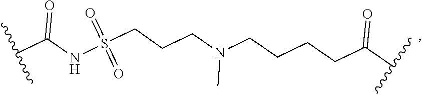 [SMILES: *C(=O)CCCCN(C)CCCS(=O)(=O)NC(*)=O] | (733) |
| 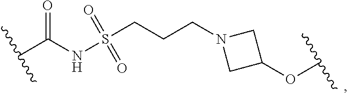 [SMILES: *OC1CN(CCCS(=O)(=O)NC(*)=O)C1] | (734) |
| 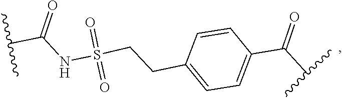 [SMILES: *C(=O)NS(=O)(=O)CCc1ccc(C(*)=O)cc1] | (735) |
| 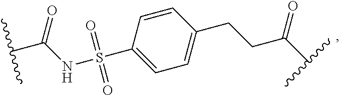 [SMILES: *C(=O)CCc1ccc(S(=O)(=O)NC(*)=O)cc1] | (736) |
| 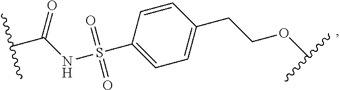 [SMILES: *OCCc1ccc(S(=O)(=O)NC(*)=O)cc1] | (737) |
| 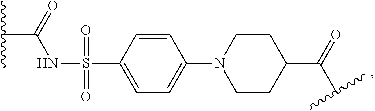 [SMILES: *C(=O)NS(=O)(=O)c1ccc(N2CCC(C(*)=O)CC2)cc1] | (738) |
| 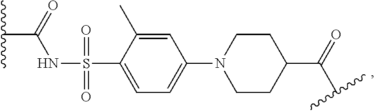 [SMILES: *C(=O)NS(=O)(=O)c1ccc(N2CCC(C(*)=O)CC2)cc1C] | (739) |
| 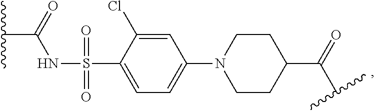 [SMILES: *C(=O)NS(=O)(=O)c1ccc(N2CCC(C(*)=O)CC2)cc1Cl] | (740) |
| 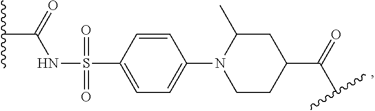 [SMILES: *C(=O)NS(=O)(=O)c1ccc(N2CCC(C(*)=O)CC2C)cc1] | (741) |
| 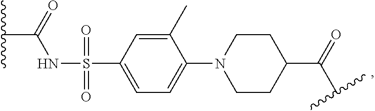 [SMILES: *C(=O)NS(=O)(=O)c1ccc(N2CCC(C(*)=O)CC2)c(C)c1] | (742) |
| 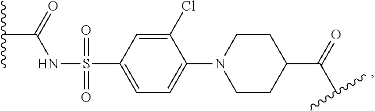 [SMILES: *C(=O)NS(=O)(=O)c1ccc(N2CCC(C(*)=O)CC2)c(Cl)c1] | (743) |
| 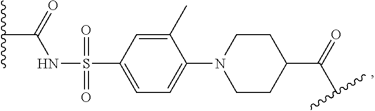 [SMILES: *C(=O)NS(=O)(=O)c1ccc(N2CCC(C(*)=O)CC2)c(C)c1] | (744) |
| 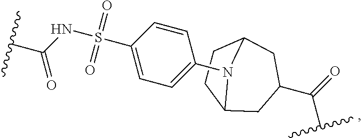 [SMILES: *C(=O)NS(=O)(=O)c1ccc(N2C3CCC2CC(C(*)=O)C3)cc1] | (745) |
| 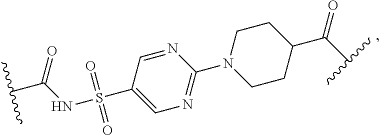 [SMILES: *C(=O)NS(=O)(=O)c1cnc(N2CCC(C(*)=O)CC2)nc1] | (746) |
| 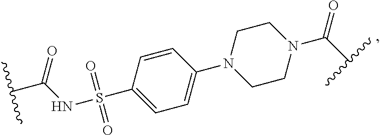 [SMILES: *C(=O)NS(=O)(=O)c1ccc(N2CCN(C(*)=O)CC2)cc1] | (747) |
| 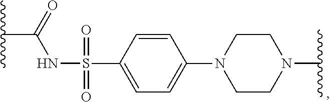 [SMILES: *C(=O)NS(=O)(=O)c1ccc(N2CCN(*)CC2)cc1] | (748) |
| 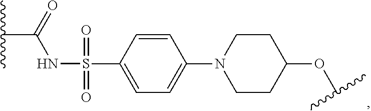 [SMILES: *OC1CCN(c2ccc(S(=O)(=O)NC(*)=O)cc2)CC1] | (749) |
| 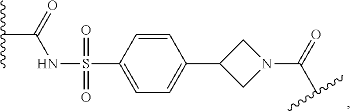 [SMILES: *C(=O)NS(=O)(=O)c1ccc(C2CN(C(*)=O)C2)cc1] | (750) |
| 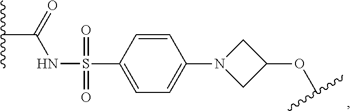 [SMILES: *OC1CN(c2ccc(S(=O)(=O)NC(*)=O)cc2)C1] | (751) |
| 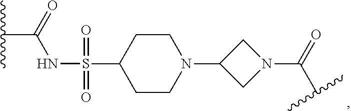 [SMILES: *C(=O)NS(=O)(=O)C1CCN(C2CN(C(*)=O)C2)CC1] | (752) |
| 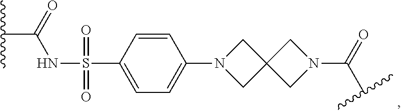 [SMILES: *C(=O)NS(=O)(=O)c1ccc(N2CC3(CN(C(*)=O)C3)C2)cc1] | (753) |
| 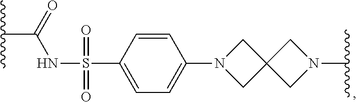 [SMILES: *C(=O)NS(=O)(=O)c1ccc(N2CC3(CN(*)C3)C2)cc1] | (754) |
| 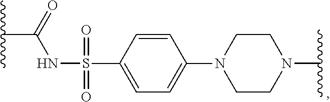 [SMILES: *C(=O)NS(=O)(=O)c1ccc(N2CCN(*)CC2)cc1] | (755) |
| 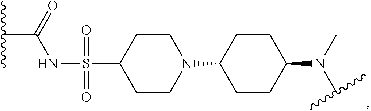 [SMILES: *C(=O)NS(=O)(=O)C1CCN([C@H]2CC[C@H](N(*)C)CC2)CC1] | (756) |
| 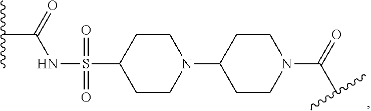 [SMILES: *C(=O)NS(=O)(=O)C1CCN(C2CCN(C(*)=O)CC2)CC1] | (757) |
| 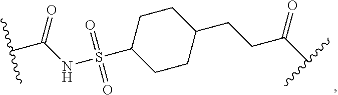 [SMILES: *C(=O)CCC1CCC(S(=O)(=O)NC(*)=O)CC1] | (758) |
| 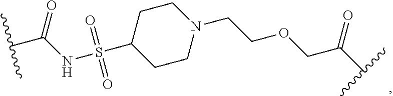 [SMILES: *C(=O)COCCN1CCC(S(=O)(=O)NC(*)=O)CC1] | (759) |
| 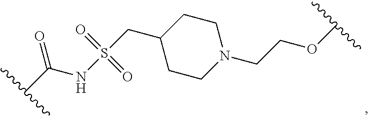 [SMILES: *OCCN1CCC(CS(=O)(=O)NC(*)=O)CC1] | (760) |
| 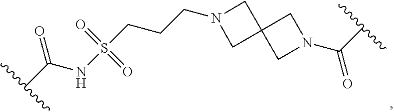 [SMILES: *C(=O)NS(=O)(=O)CCCN1CC2(C1)CN(C(*)=O)C2] | (761) |
| 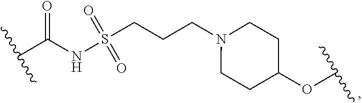 [SMILES: *OC1CCN(CCCS(=O)(=O)NC(*)=O)CC1] | (762) |
| 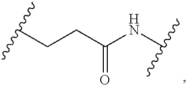 [SMILES: *CCC(=O)N*] | (763) |
| 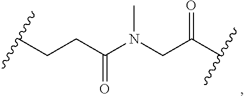 [SMILES: *CCC(=O)N(C)CC(*)=O] | (764) |
| 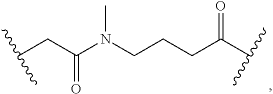 [SMILES: *CC(=O)N(C)CCCC(*)=O] | (765) |
| 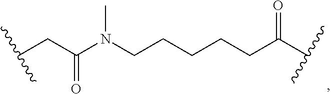 [SMILES: *CC(=O)N(C)CCCCCC(*)=O] | (766) |
| 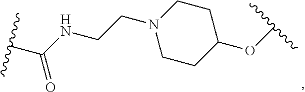 [SMILES: *OC1CCN(CCNC(*)=O)CC1] | (767) |
| 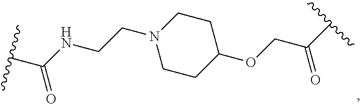 [SMILES: *C(=O)COC1CCN(CCNC(*)=O)CC1] | (768) |
| 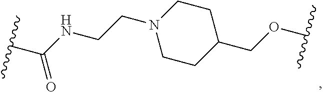 [SMILES: *OCC1CCN(CCNC(*)=O)CC1] | (769) |
| 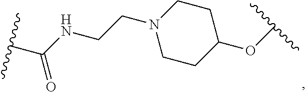 [SMILES: *OC1CCN(CCNC(*)=O)CC1] | (770) |
| 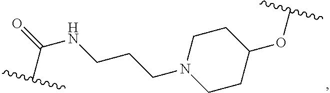 [SMILES: *OC1CCN(CCCNC(*)=O)CC1] | (771) |
| 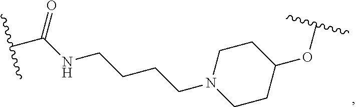 [SMILES: *OC1CCN(CCCCNC(*)=O)CC1] | (772) |
| 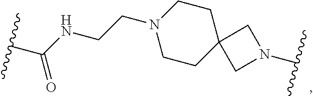 [SMILES: *C(=O)NCCN1CCC2(CC1)CN(*)C2] | (773) |
| 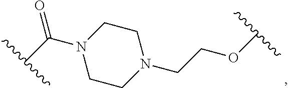 [SMILES: *OCCN1CCN(C(*)=O)CC1] | (774) |
| 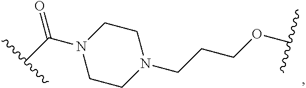 [SMILES: *OCCCN1CCN(C(*)=O)CC1] | (775) |
| 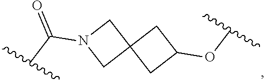 [SMILES: *OC1CC2(C1)CN(C(*)=O)C2] | (776) |
| 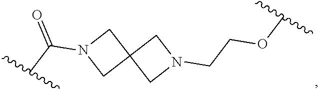 [SMILES: *OCCN1CC2(C1)CN(C(*)=O)C2] | (777) |
| 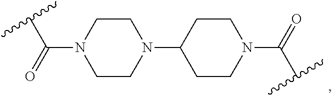 [SMILES: *C(=O)N1CCC(N2CCN(C(*)=O)CC2)CC1] | (778) |
| 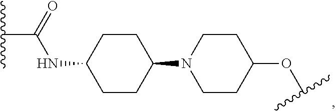 [SMILES: *OC1CCN([C@H]2CC[C@H](NC(*)=O)CC2)CC1] | (779) |
| 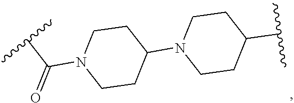 [SMILES: *C(=O)N1CCC(N2CCC(*)CC2)CC1] | (780) |
| 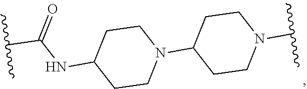 [SMILES: *C(=O)NC1CCN(C2CCN(*)CC2)CC1] | (781) |
| 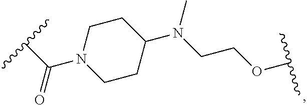 [SMILES: *OCCN(C)C1CCN(C(*)=O)CC1] | (782) |
| 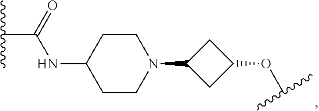 | (783) |
| 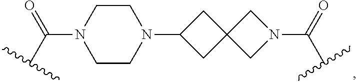 [SMILES: *C(=O)N1CCN(C2CC3(C2)CN(C(*)=O)C3)CC1] | (784) |
| 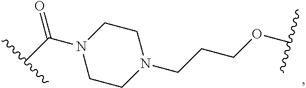 [SMILES: *OCCCN1CCN(C(*)=O)CC1] | (785) |
| 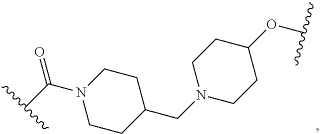 [SMILES: *OC1CCN(CC2CCN(C(*)=O)CC2)CC1] | (786) |
| 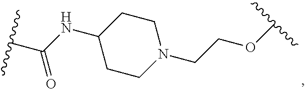 [SMILES: *OCCN1CCC(NC(*)=O)CC1] | (787) |
| 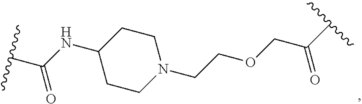 [SMILES: *C(=O)COCCN1CCC(NC(*)=O)CC1] | (788) |
| 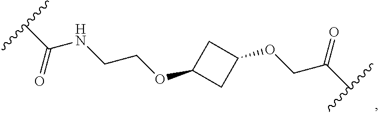 [SMILES: *C(=O)CO[C@H]1C[C@H](OCCNC(*)=O)C1] | (789) |
| 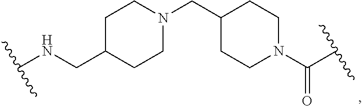 [SMILES: *NCC1CCN(CC2CCN(C(*)=O)CC2)CC1] | (790) |
| 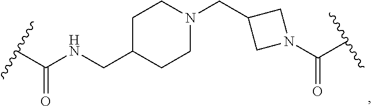 [SMILES: *C(=O)NCC1CCN(CC2CN(C(*)=O)C2)CC1] | (791) |
| 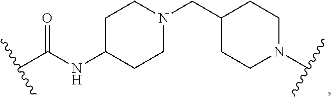 [SMILES: *C(=O)NC1CCN(CC2CCN(*)CC2)CC1] | (792) |
| 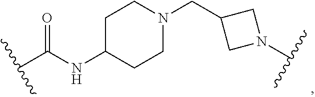 [SMILES: *C(=O)NC1CCN(CC2CN(*)C2)CC1] | (793) |
| 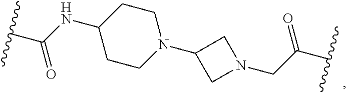 [SMILES: *C(=O)CN1CC(N2CCC(NC(*)=O)CC2)C1] | (794) |
| 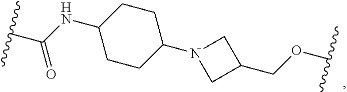 [SMILES: *OCC1CN(C2CCC(NC(*)=O)CC2)C1] | (795) |
| 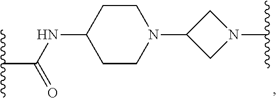 [SMILES: *C(=O)NC1CCN(C2CN(*)C2)CC1] | (796) |
| 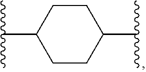 [SMILES: *C1CCC(*)CC1] | (797) |
| 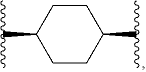 [SMILES: *[C@H]1CC[C@@H](*)CC1] | (798) |
| 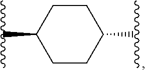 [SMILES: *[C@H]1CC[C@H](*)CC1] | (799) |
| 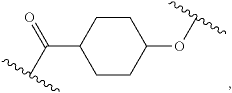 [SMILES: *OC1CCC(C(*)=O)CC1] | (800) |
| 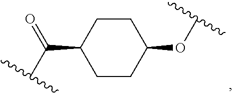 [SMILES: *O[C@H]1CC[C@@H](C(*)=O)CC1] | (801) |
| 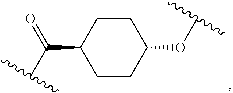 [SMILES: *O[C@H]1CC[C@H](C(*)=O)CC1] | (802) |
| 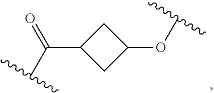 [SMILES: *OC1CC(C(*)=O)C1] | (803) |
| 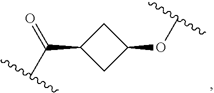 [SMILES: *O[C@H]1C[C@@H](C(*)=O)C1] | (804) |
| 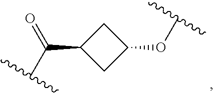 [SMILES: *O[C@H]1C[C@H](C(*)=O)C1] | (805) |
| 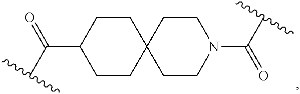 [SMILES: *C(=O)C1CCC2(CC1)CCN(C(*)=O)CC2] | (806) |
| 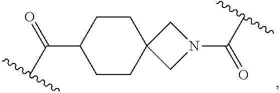 [SMILES: *C(=O)C1CCC2(CC1)CN(C(*)=O)C2] | (807) |
| 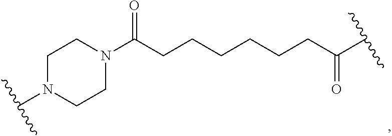 [SMILES: *C(=O)CCCCCCC(=O)N1CCN(*)CC1] | (808) |
| 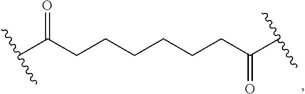 [SMILES: *C(=O)CCCCCCC(*)=O] | (809) |
| 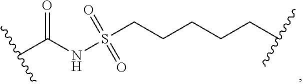 [SMILES: *CCCCCS(=O)(=O)NC(*)=O] | (810) |
| 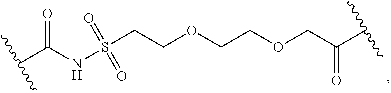 [SMILES: *C(=O)COCCOCCS(=O)(=O)NC(*)=O] | (811) |
| 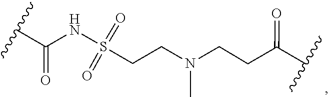 [SMILES: *C(=O)CCN(C)CCS(=O)(=O)NC(*)=O] | (812) |
| 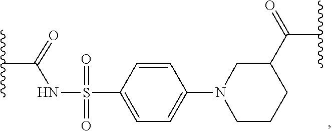 [SMILES: *C(=O)NS(=O)(=O)c1ccc(N2CCCC(C(*)=O)C2)cc1] | (813) |
| 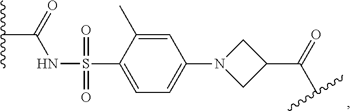 [SMILES: *C(=O)NS(=O)(=O)c1ccc(N2CC(C(*)=O)C2)cc1C] | (814) |
| 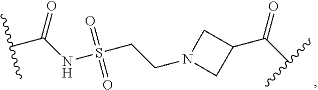 [SMILES: *C(=O)NS(=O)(=O)CCN1CC(C(*)=O)C1] | (815) |
| 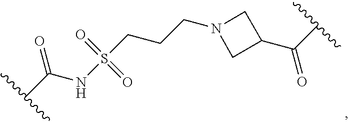 [SMILES: *C(=O)NS(=O)(=O)CCCN1CC(C(*)=O)C1] | (816) |
| 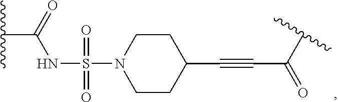 [SMILES: *C(=O)C#CC1CCN(S(=O)(=O)NC(*)=O)CC1] | (817) |
| 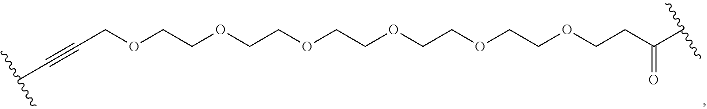 [SMILES: *C#CCOCCOCCOCCOCCOCCOCCC(*)=O] | (818) |
| 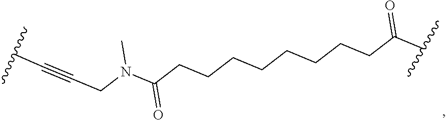 [SMILES: *C#CCN(C)C(=O)CCCCCCCCC(*)=O] | (819) |
| 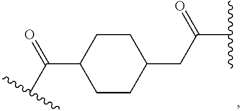 [SMILES: *C(=O)CC1CCC(C(*)=O)CC1] | (820) |
| 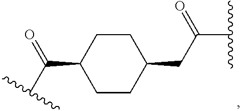 [SMILES: *C(=O)C[C@H]1CC[C@@H](C(*)=O)CC1] | (821) |
| 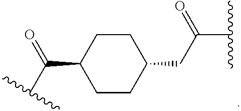 [SMILES: *C(=O)C[C@H]1CC[C@H](C(*)=O)CC1] | (822) |

In some embodiments, the present invention provides a compound having BBM described and disclosed herein. LBM set forth in Table A above, and a linker set forth in Table B above, or a pharmaceutically acceptable salt thereof.

Exemplary compounds of the invention are set forth in Table 1, below.

TABLE 1
Exemplary Compounds

| I-# | Structure |
|---|---|
| I-1 | 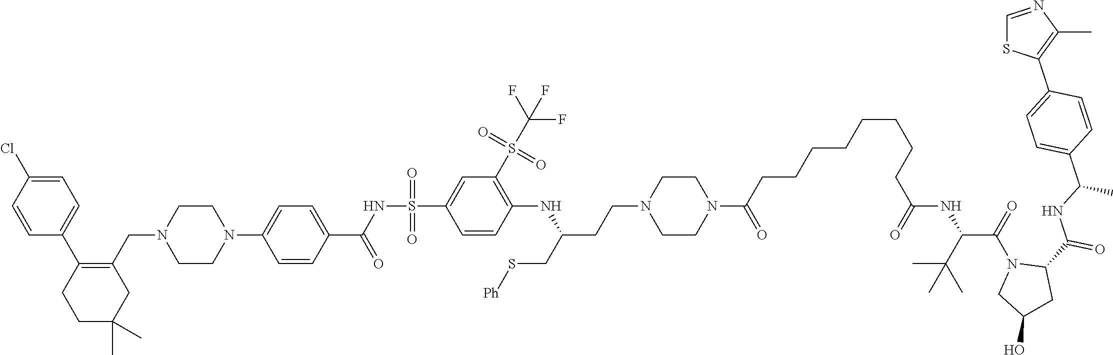 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-2 | 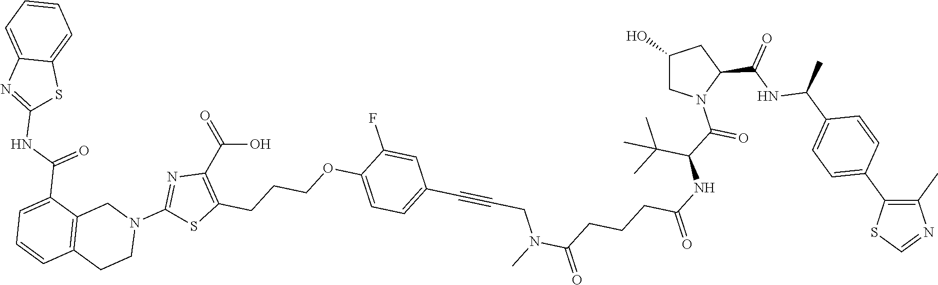 [SMILES: Cc1ncsc1-c1ccc([C@H](C)NC(=O)[C@@H]2C[C@@H](O)CN2C(=O)[C@@H](NC(=O)CCCC(=O)N(C)CC#Cc2ccc(OCCCc3sc(N4CCc5cccc(C(=O)Nc6nc7ccccc7s6)c5C4)nc3C(=O)O)c(F)c2)C(C)(C)C)cc1] |
| I-3 | 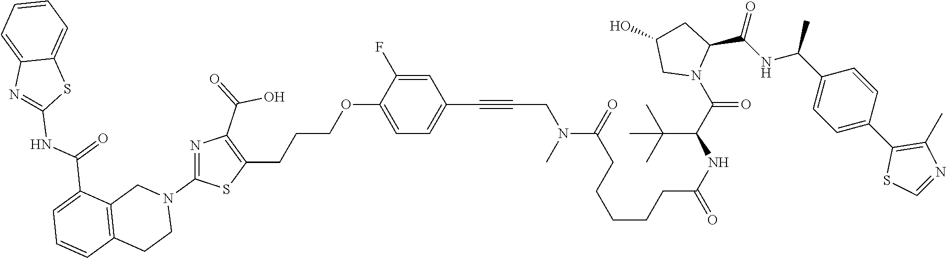 [SMILES: Cc1ncsc1-c1ccc([C@H](C)NC(=O)[C@@H]2C[C@@H](O)CN2C(=O)[C@@H](NC(=O)CCCCCC(=O)N(C)CC#Cc2ccc(OCCCc3sc(N4CCc5cccc(C(=O)Nc6nc7ccccc7s6)c5C4)nc3C(=O)O)c(F)c2)C(C)(C)C)cc1] |
| I-4 | 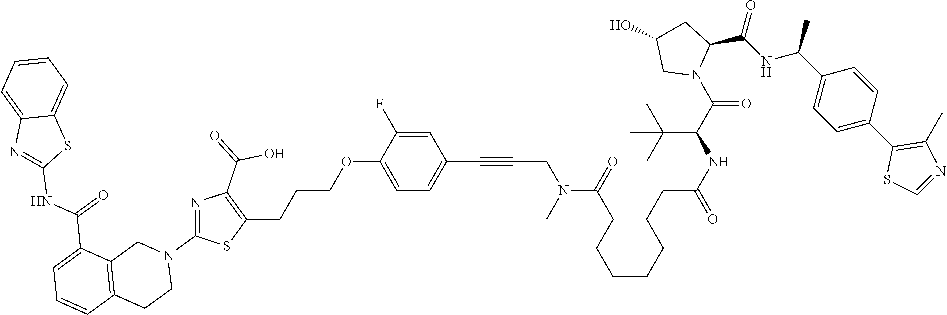 [SMILES: Cc1ncsc1-c1ccc([C@H](C)NC(=O)[C@@H]2C[C@@H](O)CN2C(=O)[C@@H](NC(=O)CCCCCCCC(=O)N(C)CC#Cc2ccc(OCCCc3sc(N4CCc5cccc(C(=O)Nc6nc7ccccc7s6)c5C4)nc3C(=O)O)c(F)c2)C(C)(C)C)cc1] |
| I-5 | 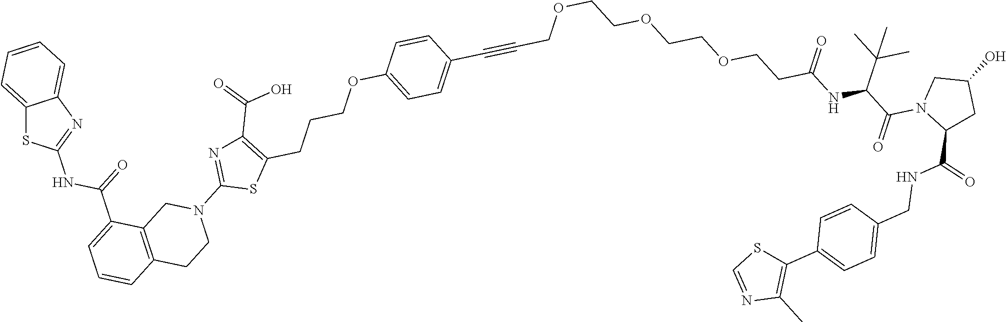 [SMILES: Cc1ncsc1-c1ccc(CNC(=O)[C@@H]2C[C@@H](O)CN2C(=O)[C@@H](NC(=O)CCOCCOCCOCC#Cc2ccc(OCCCc3sc(N4CCc5cccc(C(=O)Nc6nc7ccccc7s6)c5C4)nc3C(=O)O)cc2)C(C)(C)C)cc1] |
| I-6 | 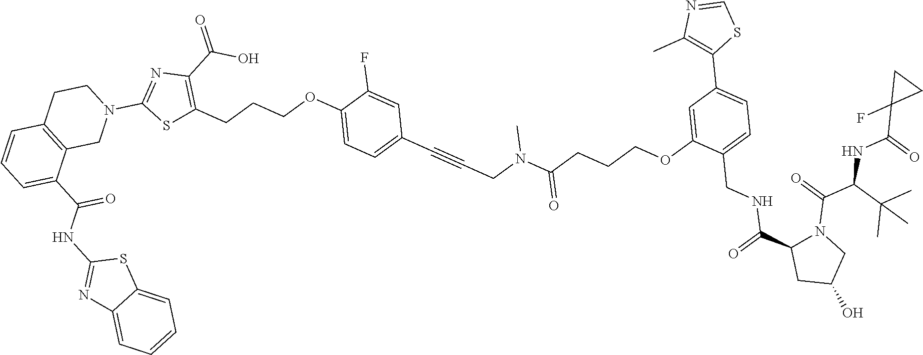 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N(C)CC#Cc2ccc(OCCCc3sc(N4CCc5cccc(C(=O)Nc6nc7ccccc7s6)c5C4)nc3C(=O)O)c(F)c2)c1] |
| I-7 | 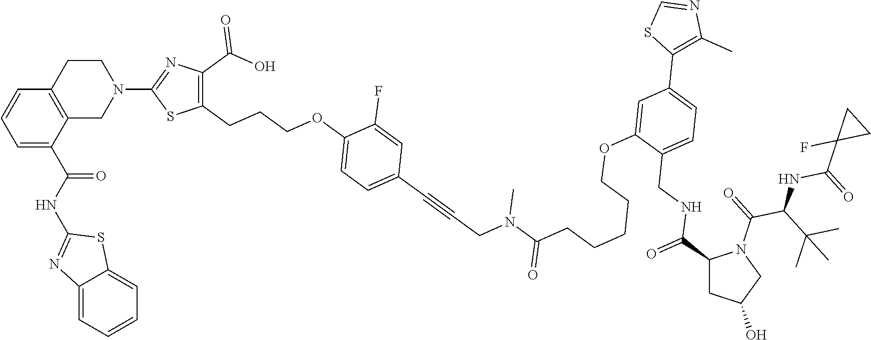 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N(C)CC#Cc2ccc(OCCCc3sc(N4CCc5cccc(C(=O)Nc6nc7ccccc7s6)c5C4)nc3C(=O)O)c(F)c2)c1] |
| I-8 | 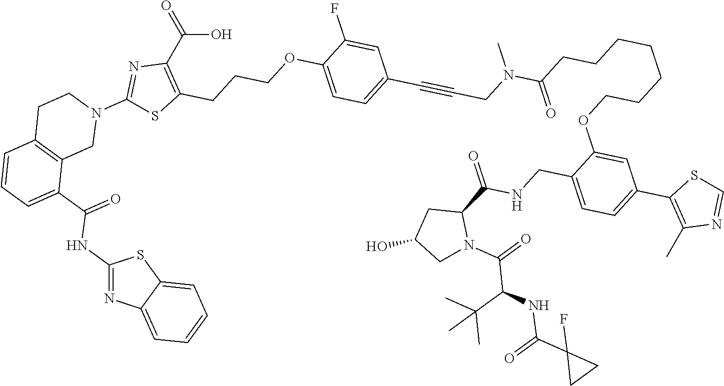 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N(C)CC#Cc2ccc(OCCCc3sc(N4CCc5cccc(C(=O)Nc6nc7ccccc7s6)c5C4)nc3C(=O)O)c(F)c2)c1] |
| I-9 | 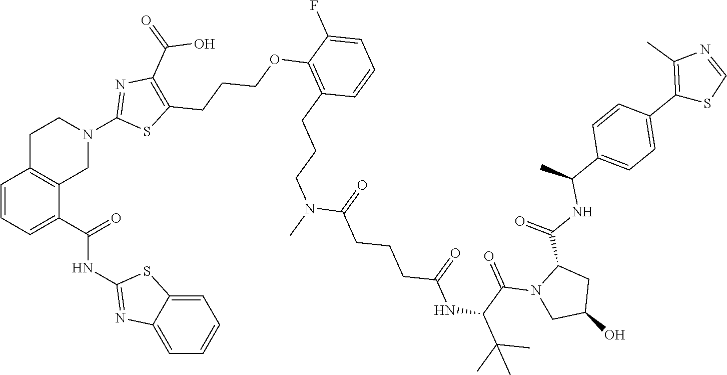 [SMILES: Cc1ncsc1-c1ccc([C@H](C)NC(=O)[C@@H]2C[C@@H](O)CN2C(=O)[C@@H](NC(=O)CCCC(=O)N(C)CCCc2cccc(F)c2OCCCc2sc(N3CCc4cccc(C(=O)Nc5nc6ccccc6s5)c4C3)nc2C(=O)O)C(C)(C)C)cc1] |
| I-10 | 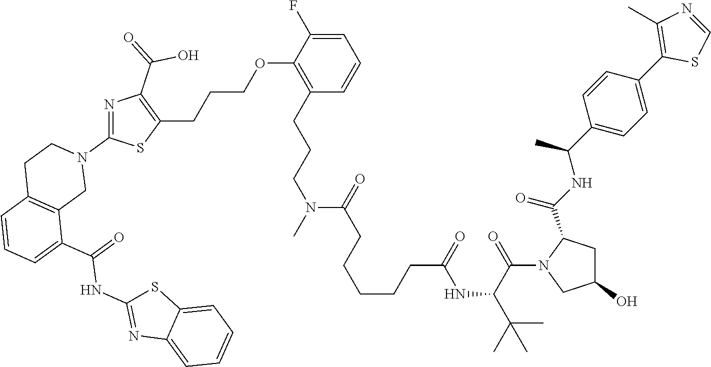 [SMILES: Cc1ncsc1-c1ccc([C@H](C)NC(=O)[C@@H]2C[C@@H](O)CN2C(=O)[C@@H](NC(=O)CCCCCC(=O)N(C)CCCc2cccc(F)c2OCCCc2sc(N3CCc4cccc(C(=O)Nc5nc6ccccc6s5)c4C3)nc2C(=O)O)C(C)(C)C)cc1] |
| I-11 | 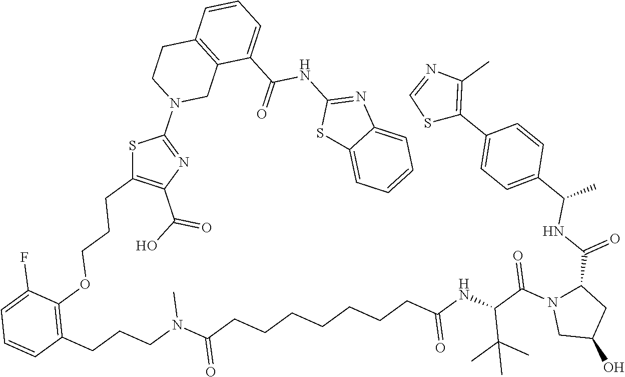 [SMILES: Cc1ncsc1-c1ccc([C@H](C)NC(=O)[C@@H]2C[C@@H](O)CN2C(=O)[C@@H](NC(=O)CCCCCCCC(=O)N(C)CCCc2cccc(F)c2OCCCc2sc(N3CCc4cccc(C(=O)Nc5nc6ccccc6s5)c4C3)nc2C(=O)O)C(C)(C)C)cc1] |
| I-12 | 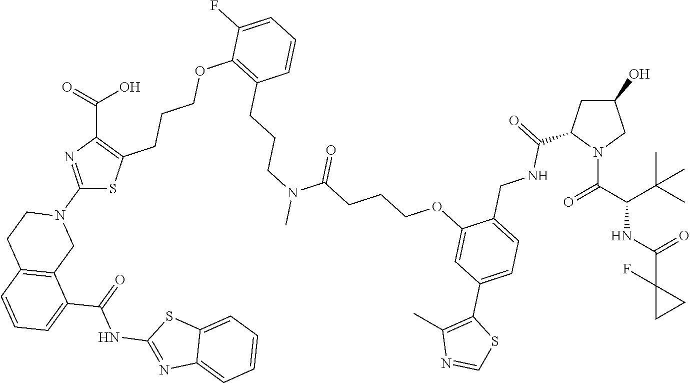 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N(C)CCCc2cccc(F)c2OCCCc2sc(N3CCc4cccc(C(=O)Nc5nc6ccccc6s5)c4C3)nc2C(=O)O)c1] |
| I-13 | 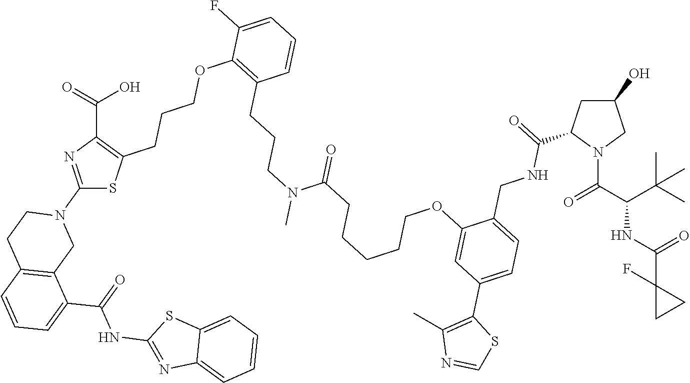 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N(C)CCCc2cccc(F)c2OCCCc2sc(N3CCc4cccc(C(=O)Nc5nc6ccccc6s5)c4C3)nc2C(=O)O)c1] |
| I-14 | 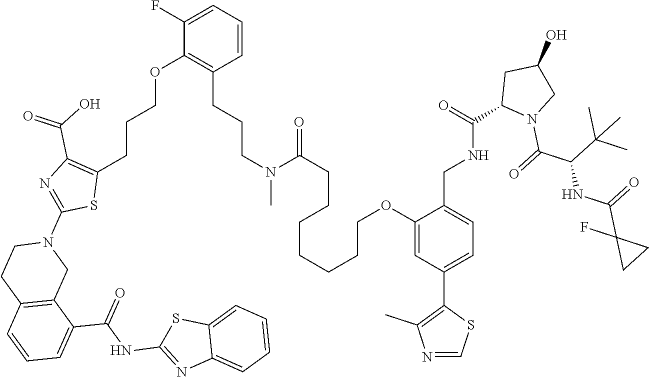 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N(C)CCCc2cccc(F)c2OCCCc2sc(N3CCc4cccc(C(=O)Nc5nc6ccccc6s5)c4C3)nc2C(=O)O)c1] |
| I-15 | 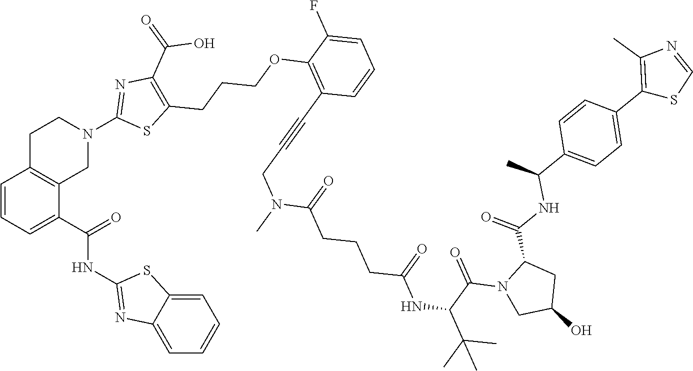 [SMILES: Cc1ncsc1-c1ccc([C@H](C)NC(=O)[C@@H]2C[C@@H](O)CN2C(=O)[C@@H](NC(=O)CCCC(=O)N(C)CC#Cc2cccc(F)c2OCCCc2sc(N3CCc4cccc(C(=O)Nc5nc6ccccc6s5)c4C3)nc2C(=O)O)C(C)(C)C)cc1] |
| I-16 | 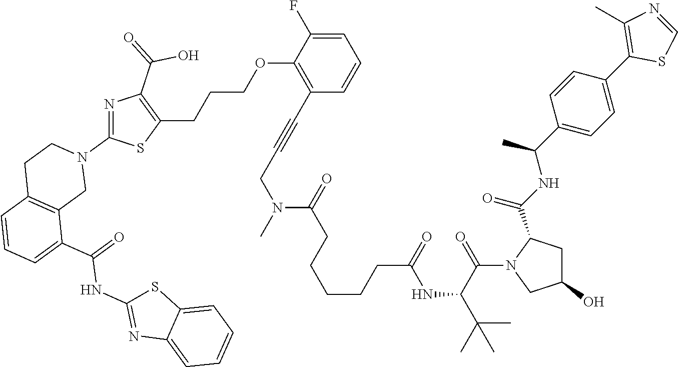 [SMILES: Cc1ncsc1-c1ccc([C@H](C)NC(=O)[C@@H]2C[C@@H](O)CN2C(=O)[C@@H](NC(=O)CCCCCC(=O)N(C)CC#Cc2cccc(F)c2OCCCc2sc(N3CCc4cccc(C(=O)Nc5nc6ccccc6s5)c4C3)nc2C(=O)O)C(C)(C)C)cc1] |
| I-17 | 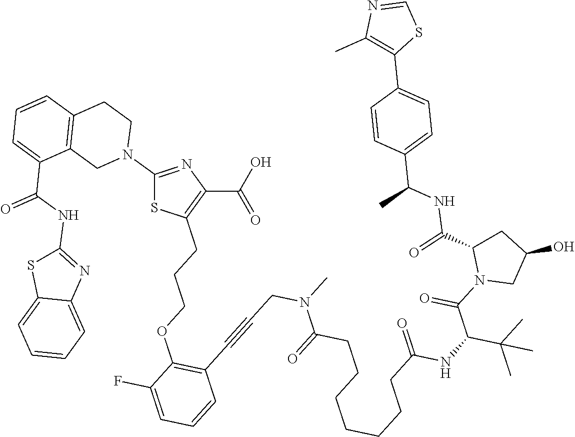 [SMILES: Cc1ncsc1-c1ccc([C@H](C)NC(=O)[C@@H]2C[C@@H](O)CN2C(=O)[C@@H](NC(=O)CCCCCCCC(=O)N(C)CC#Cc2cccc(F)c2OCCCc2sc(N3CCc4cccc(C(=O)Nc5nc6ccccc6s5)c4C3)nc2C(=O)O)C(C)(C)C)cc1] |
| I-18 | 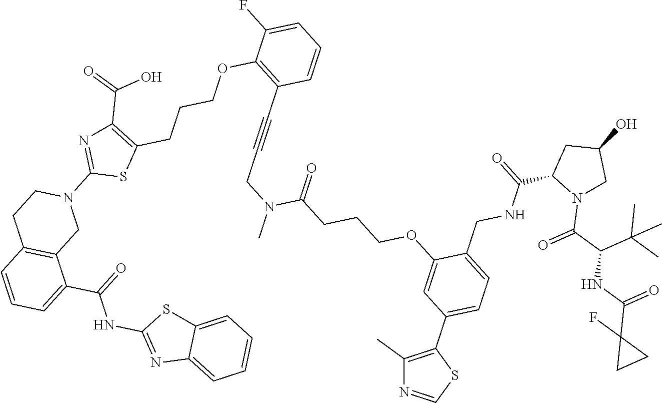 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N(C)CC#Cc2cccc(F)c2OCCCc2sc(N3CCc4cccc(C(=O)Nc5nc6ccccc6s5)c4C3)nc2C(=O)O)c1] |
| I-19 | 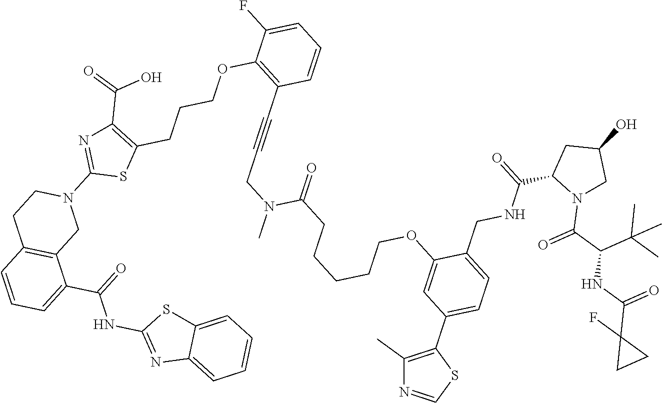 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N(C)CC#Cc2cccc(F)c2OCCCc2sc(N3CCc4cccc(C(=O)Nc5nc6ccccc6s5)c4C3)nc2C(=O)O)c1] |
| I-20 | 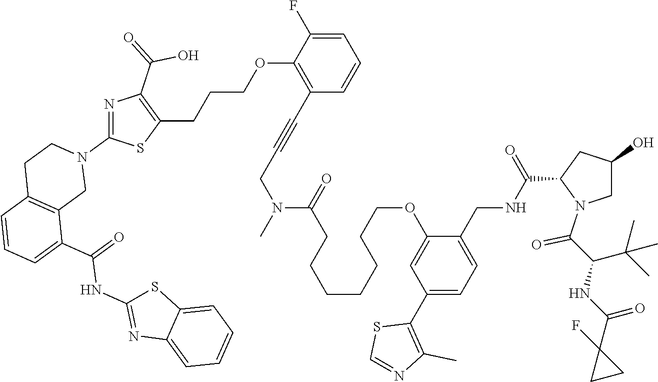 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N(C)CC#Cc2cccc(F)c2OCCCc2sc(N3CCc4cccc(C(=O)Nc5nc6ccccc6s5)c4C3)nc2C(=O)O)c1] |
| I-21 | 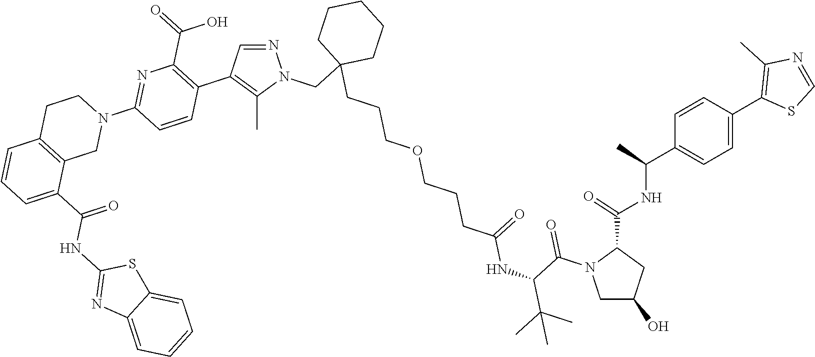 [SMILES: Cc1ncsc1-c1ccc([C@H](C)NC(=O)[C@@H]2C[C@@H](O)CN2C(=O)[C@@H](NC(=O)CCCOCCCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-22 | 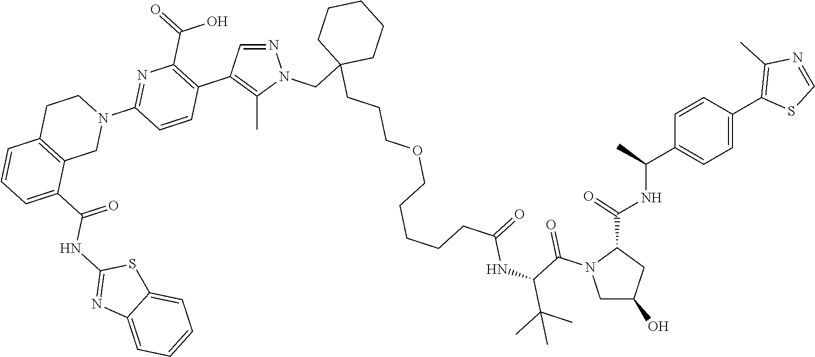 [SMILES: Cc1ncsc1-c1ccc([C@H](C)NC(=O)[C@@H]2C[C@@H](O)CN2C(=O)[C@@H](NC(=O)CCCCCOCCCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-23 | 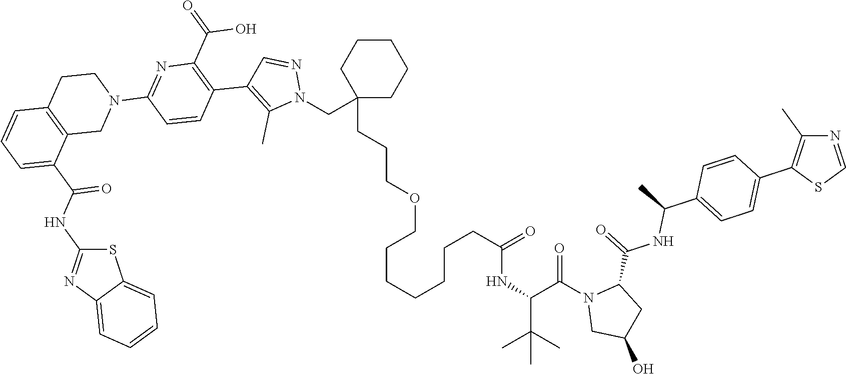 [SMILES: Cc1ncsc1-c1ccc([C@H](C)NC(=O)[C@@H]2C[C@@H](O)CN2C(=O)[C@@H](NC(=O)CCCCCCCOCCCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-24 | 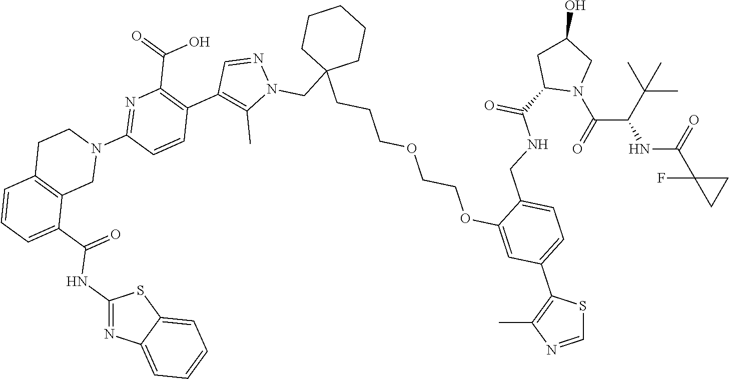 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCC2(Cn3ncc(-c4ccc(N5CCc6cccc(C(=O)Nc7nc8ccccc8s7)c6C5)nc4C(=O)O)c3C)CCCCC2)c1] |
| I-25 | 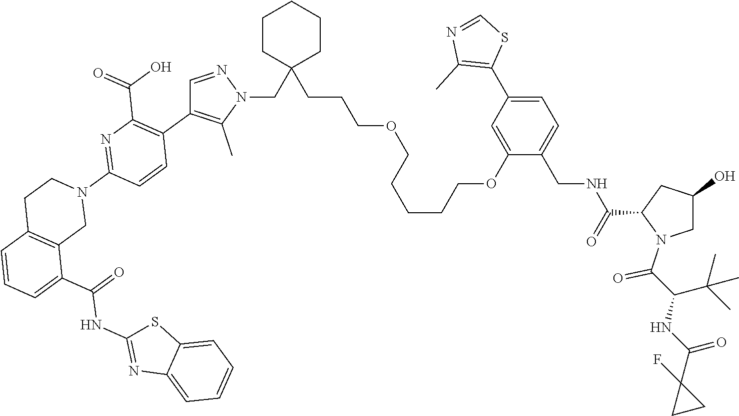 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCOCCCC2(Cn3ncc(-c4ccc(N5CCc6cccc(C(=O)Nc7nc8ccccc8s7)c6C5)nc4C(=O)O)c3C)CCCCC2)c1] |
| I-26 | 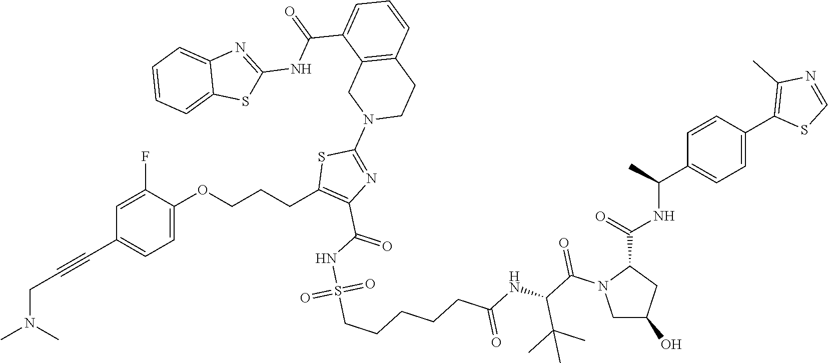 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)sc2CCCOc2ccc(C#CCN(C)C)cc2F)C(C)(C)C)cc1] |
| I-27 | 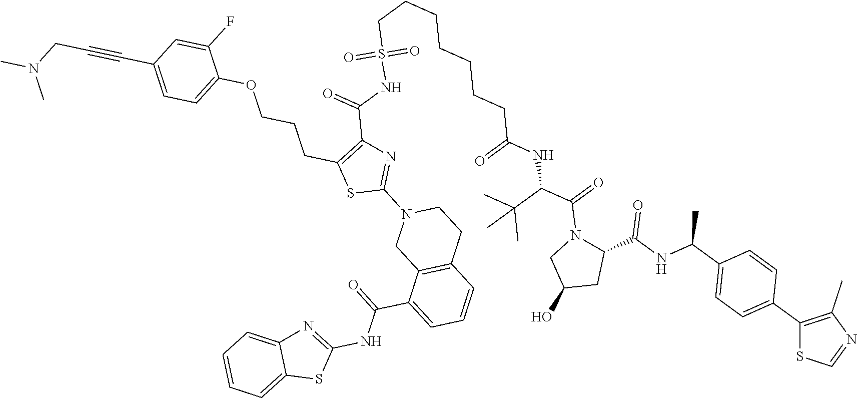 [SMILES: Cc1ncsc1-c1ccc([C@H](C)NC(=O)[C@@H]2C[C@@H](O)CN2C(=O)[C@@H](NC(=O)CCCCCCCS(=O)(=O)NC(=O)c2nc(N3CCc4cccc(C(=O)Nc5nc6ccccc6s5)c4C3)sc2CCCOc2ccc(C#CCN(C)C)cc2F)C(C)(C)C)cc1] |
| I-28 | 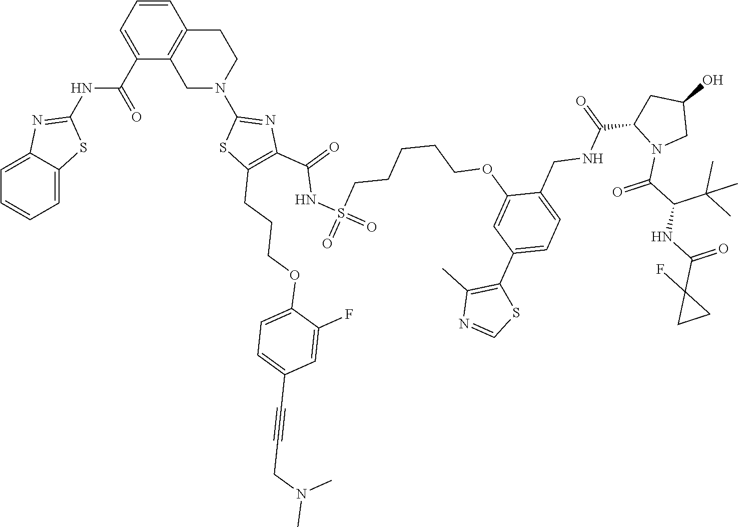 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCS(=O)(=O)NC(=O)c2nc(N3CCc4cccc(C(=O)Nc5nc6ccccc6s5)c4C3)sc2CCCOc2ccc(C#CCN(C)C)cc2F)c1] |
| I-29 | 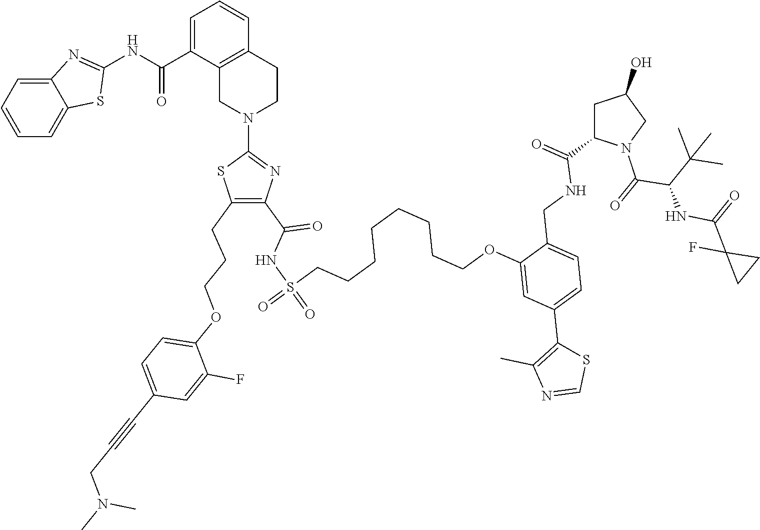 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCS(=O)(=O)NC(=O)c2nc(N3CCc4cccc(C(=O)Nc5nc6ccccc6s5)c4C3)sc2CCCOc2ccc(C#CCN(C)C)cc2F)c1] |
| I-30 | 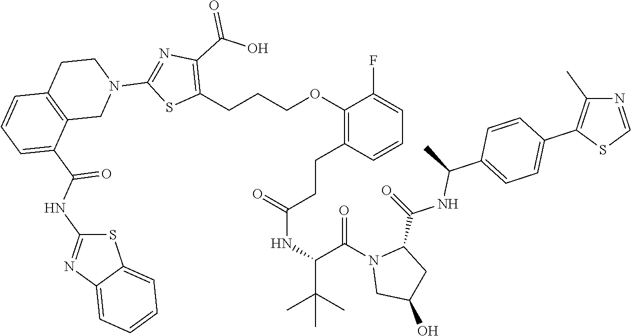 [SMILES: Cc1ncsc1-c1ccc([C@H](C)NC(=O)[C@@H]2C[C@@H](O)CN2C(=O)[C@@H](NC(=O)CCc2cccc(F)c2OCCCc2sc(N3CCc4cccc(C(=O)Nc5nc6ccccc6s5)c4C3)nc2C(=O)O)C(C)(C)C)cc1] |
| I-31 | 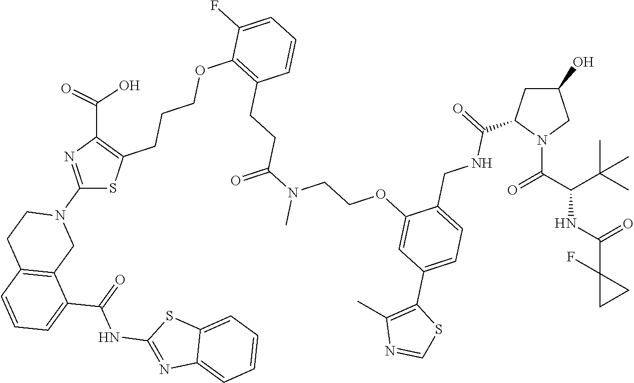 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN(C)C(=O)CCc2cccc(F)c2OCCCc2sc(N3CCc4cccc(C(=O)Nc5nc6ccccc6s5)c4C3)nc2C(=O)O)c1] |
| I-32 | 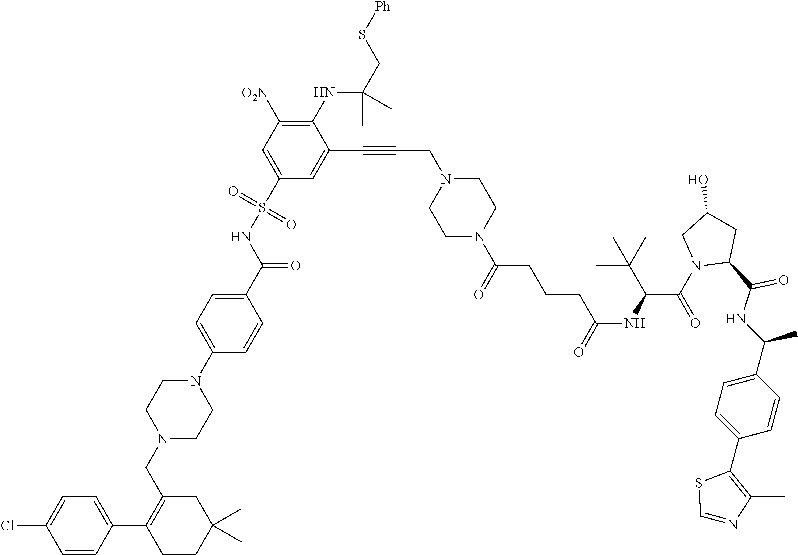 [SMILES: Cc1ncsc1-c1ccc([C@H](C)NC(=O)[C@@H]2C[C@@H](O)CN2C(=O)[C@@H](NC(=O)CCCC(=O)N2CCN(CC#Cc3cc(S(=O)(=O)NC(=O)c4ccc(N5CCN(CC6=C(c7ccc(Cl)cc7)CCC(C)(C)C6)CC5)cc4)cc([N+](=O)[O-])c3NC(C)(C)CSc3ccccc3)CC2)C(C)(C)C)cc1] |
| I-33 | 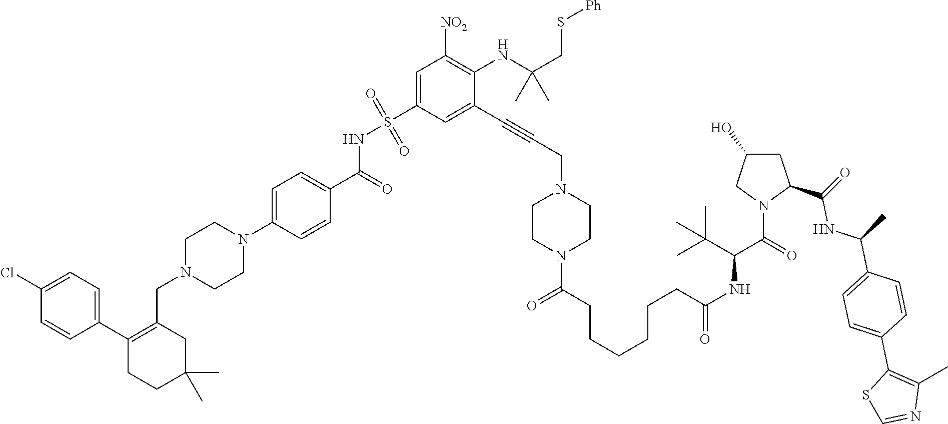 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC#Cc3cc(S(=O)(=O)NC(=O)c4ccc(N5CCN(CC6=C(c7ccc(Cl)cc7)CCC(C)(C)C6)CC5)cc4)cc([N+](=O)[O-])c3NC(C)(C)CSc3ccccc3)CC2)C(C)(C)C)cc1] |
| I-34 | 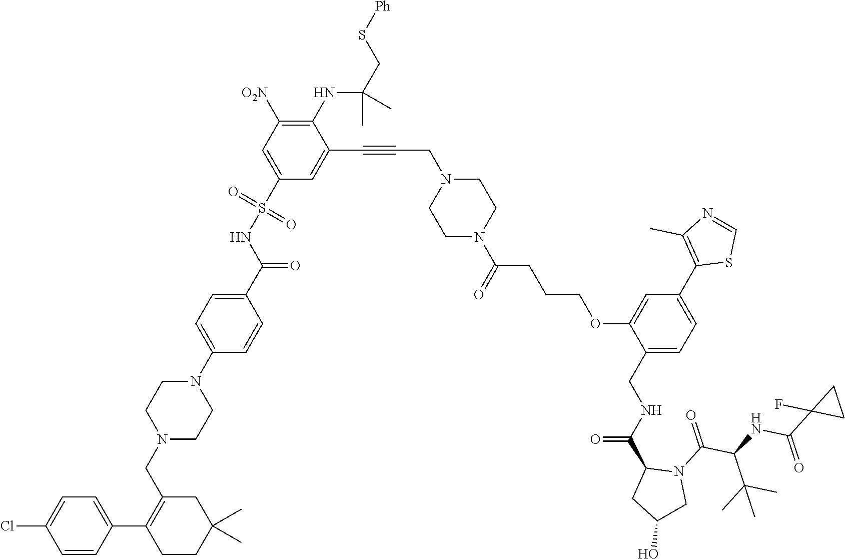 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CCN(CC#Cc3cc(S(=O)(=O)NC(=O)c4ccc(N5CCN(CC6=C(c7ccc(Cl)cc7)CCC(C)(C)C6)CC5)cc4)cc([N+](=O)[O-])c3NC(C)(C)CSc3ccccc3)CC2)c1] |
| I-35 | 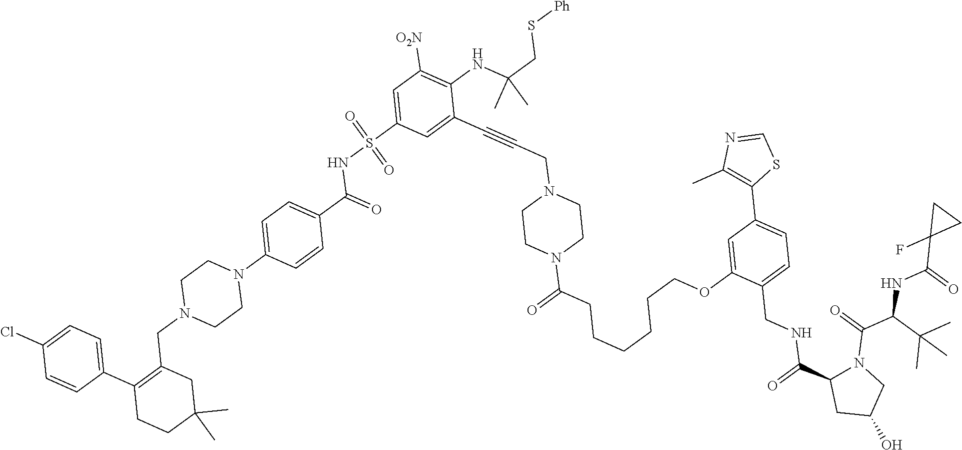 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCN(CC#Cc3cc(S(=O)(=O)NC(=O)c4ccc(N5CCN(CC6=C(c7ccc(Cl)cc7)CCC(C)(C)C6)CC5)cc4)cc([N+](=O)[O-])c3NC(C)(C)CSc3ccccc3)CC2)c1] |
| I-36 | 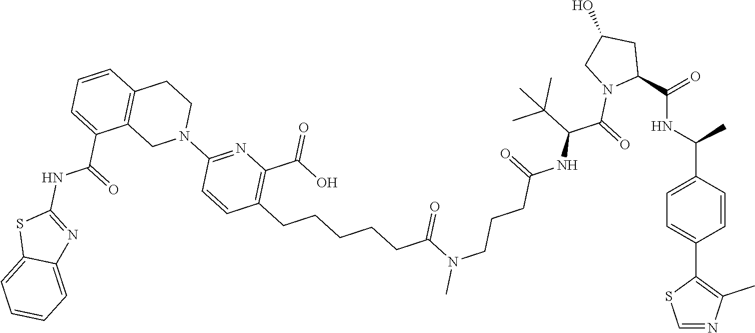 [SMILES: Cc1ncsc1-c1ccc([C@H](C)NC(=O)[C@@H]2C[C@@H](O)CN2C(=O)[C@@H](NC(=O)CCCN(C)C(=O)CCCCCc2ccc(N3CCc4cccc(C(=O)Nc5nc6ccccc6s5)c4C3)nc2C(=O)O)C(C)(C)C)cc1] |
| I-37 | 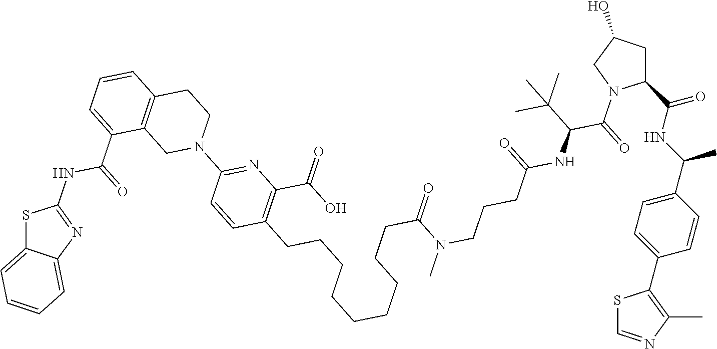 [SMILES: Cc1ncsc1-c1ccc([C@H](C)NC(=O)[C@@H]2C[C@@H](O)CN2C(=O)[C@@H](NC(=O)CCCN(C)C(=O)CCCCCCCCCc2ccc(N3CCc4cccc(C(=O)Nc5nc6ccccc6s5)c4C3)nc2C(=O)O)C(C)(C)C)cc1] |
| I-38 | 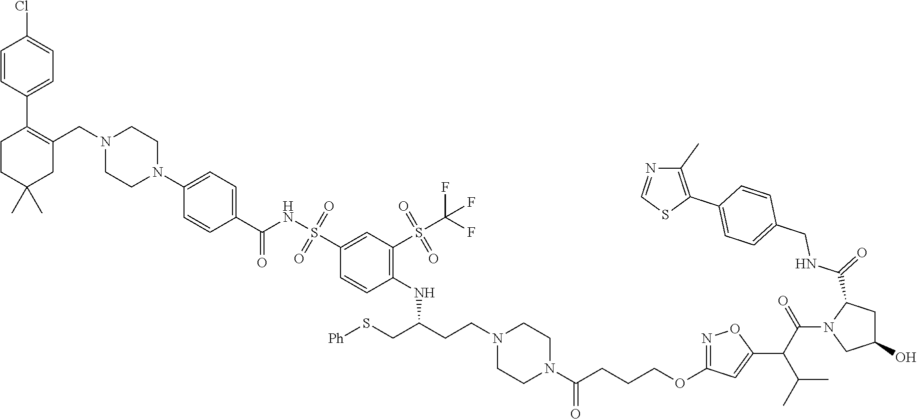 [SMILES: Cc1ncsc1-c1ccc(CNC(=O)[C@@H]2C[C@@H](O)CN2C(=O)C(c2cc(OCCCC(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)no2)C(C)C)cc1] |
| I-39 | 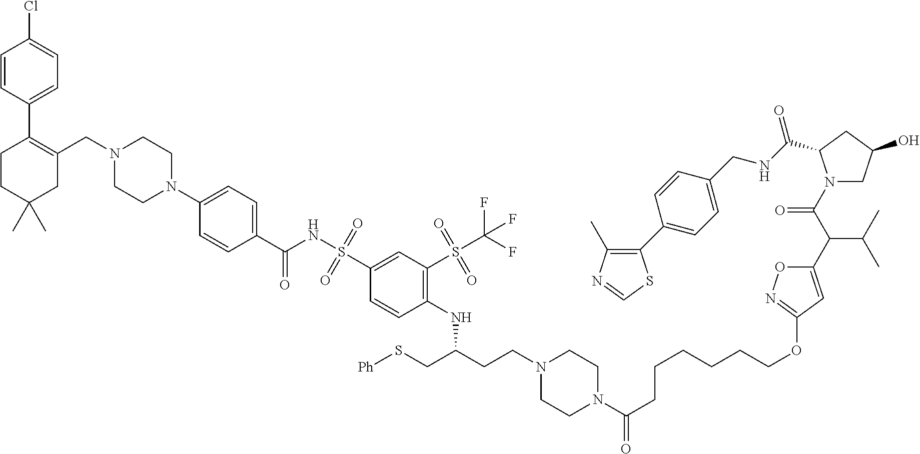 [SMILES: Cc1ncsc1-c1ccc(CNC(=O)[C@@H]2C[C@@H](O)CN2C(=O)C(c2cc(OCCCCCCC(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)no2)C(C)C)cc1] |
| I-40 | 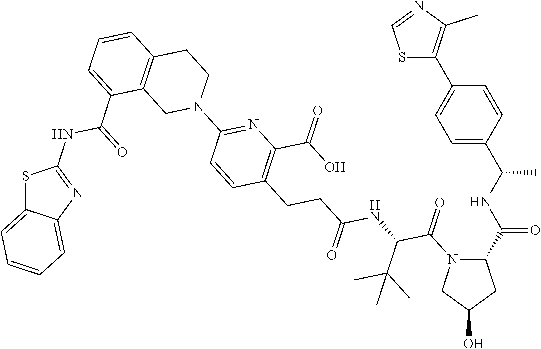 [SMILES: Cc1ncsc1-c1ccc([C@H](C)NC(=O)[C@@H]2C[C@@H](O)CN2C(=O)[C@@H](NC(=O)CCc2ccc(N3CCc4cccc(C(=O)Nc5nc6ccccc6s5)c4C3)nc2C(=O)O)C(C)(C)C)cc1] |
| I-41 | 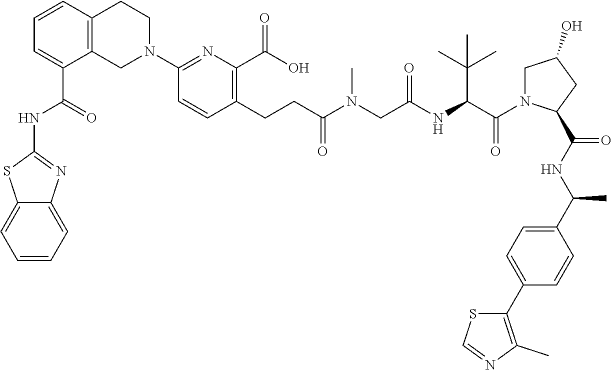 [SMILES: Cc1ncsc1-c1ccc([C@H](C)NC(=O)[C@@H]2C[C@@H](O)CN2C(=O)[C@@H](NC(=O)CN(C)C(=O)CCc2ccc(N3CCc4cccc(C(=O)Nc5nc6ccccc6s5)c4C3)nc2C(=O)O)C(C)(C)C)cc1] |
| I-42 | 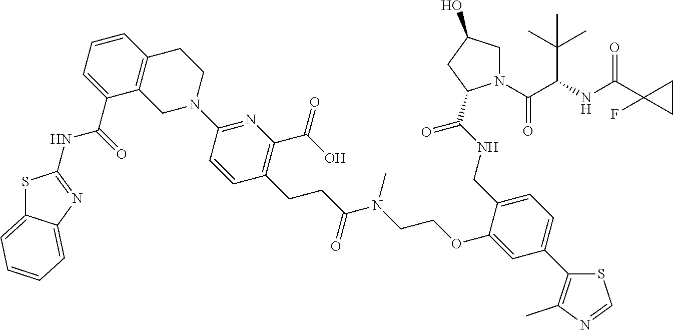 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN(C)C(=O)CCc2ccc(N3CCc4cccc(C(=O)Nc5nc6ccccc6s5)c4C3)nc2C(=O)O)c1] |
| I-43 | 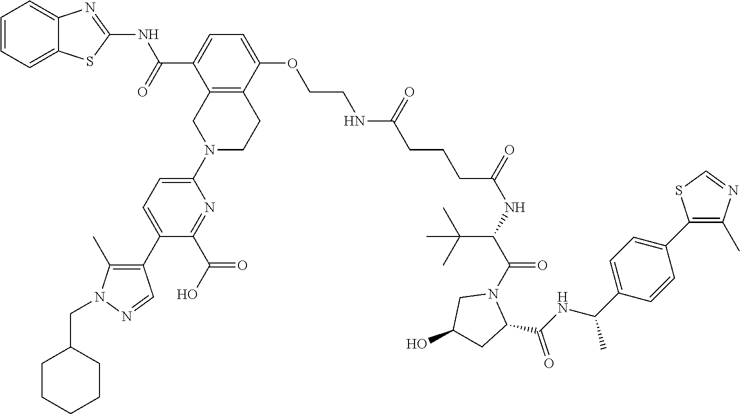 [SMILES: Cc1ncsc1-c1ccc([C@H](C)NC(=O)[C@@H]2C[C@@H](O)CN2C(=O)[C@@H](NC(=O)CCCC(=O)NCCOc2ccc(C(=O)Nc3nc4ccccc4s3)c3c2CCN(c2ccc(-c4cnn(CC5CCCCC5)c4C)c(C(=O)O)n2)C3)C(C)(C)C)cc1] |
| I-44 | 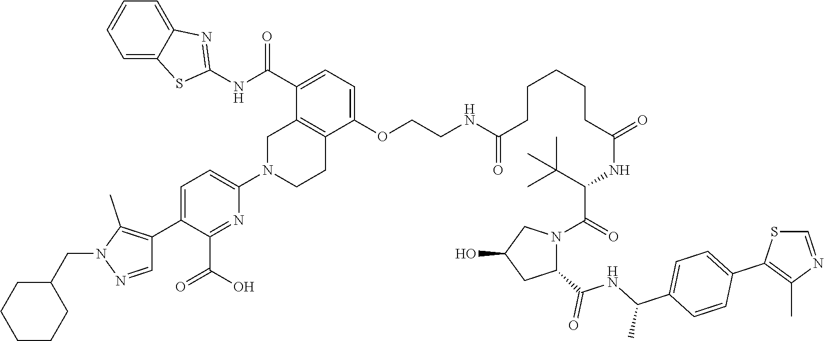 [SMILES: Cc1ncsc1-c1ccc([C@H](C)NC(=O)[C@@H]2C[C@@H](O)CN2C(=O)[C@@H](NC(=O)CCCCCC(=O)NCCOc2ccc(C(=O)Nc3nc4ccccc4s3)c3c2CCN(c2ccc(-c4cnn(CC5CCCCC5)c4C)c(C(=O)O)n2)C3)C(C)(C)C)cc1] |
| I-45 | 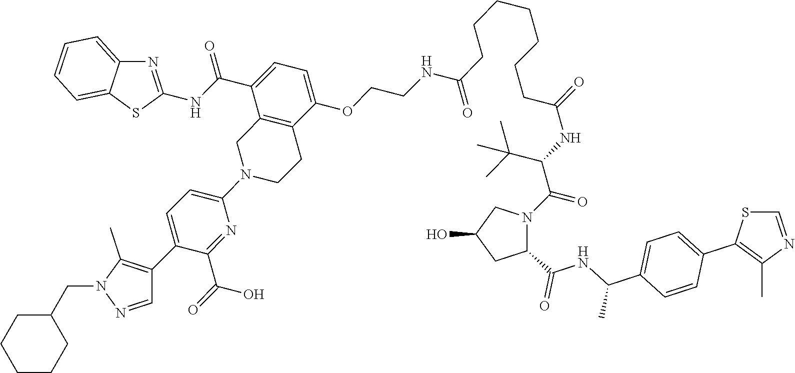 [SMILES: Cc1ncsc1-c1ccc([C@H](C)NC(=O)[C@@H]2C[C@@H](O)CN2C(=O)[C@@H](NC(=O)CCCCCCCC(=O)NCCOc2ccc(C(=O)Nc3nc4ccccc4s3)c3c2CCN(c2ccc(-c4cnn(CC5CCCCC5)c4C)c(C(=O)O)n2)C3)C(C)(C)C)cc1] |
| I-46 | 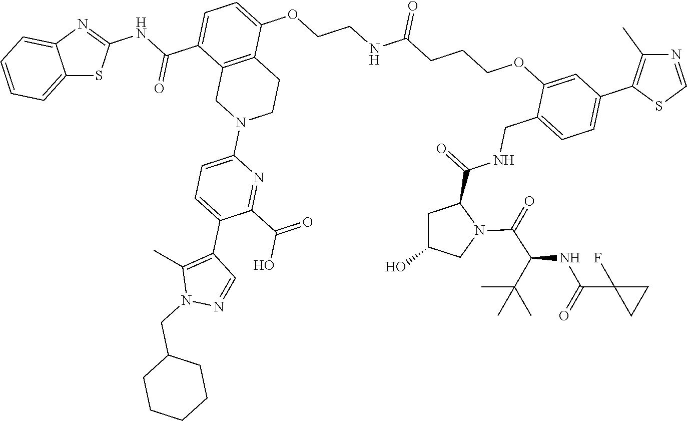 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)NCCOc2ccc(C(=O)Nc3nc4ccccc4s3)c3c2CCN(c2ccc(-c4cnn(CC5CCCCC5)c4C)c(C(=O)O)n2)C3)c1] |
| I-47 | 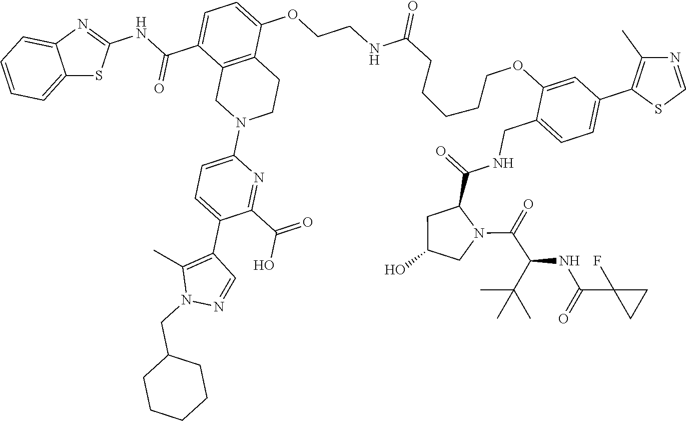 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)NCCOc2ccc(C(=O)Nc3nc4ccccc4s3)c3c2CCN(c2ccc(-c4cnn(CC5CCCCC5)c4C)c(C(=O)O)n2)C3)c1] |
| I-48 | 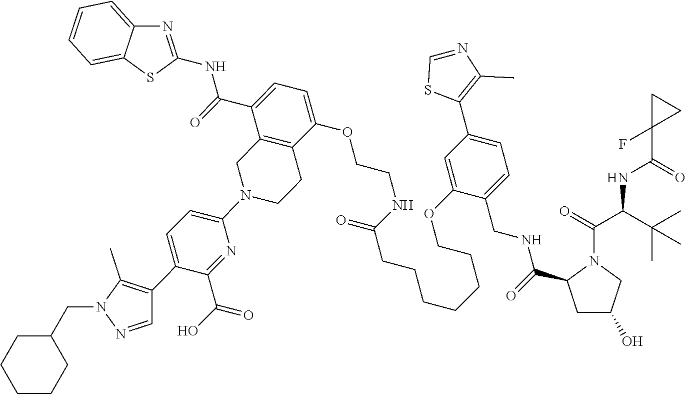 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)NCCOc2ccc(C(=O)Nc3nc4ccccc4s3)c3c2CCN(c2ccc(-c4cnn(CC5CCCCC5)c4C)c(C(=O)O)n2)C3)c1] |
| I-49 | 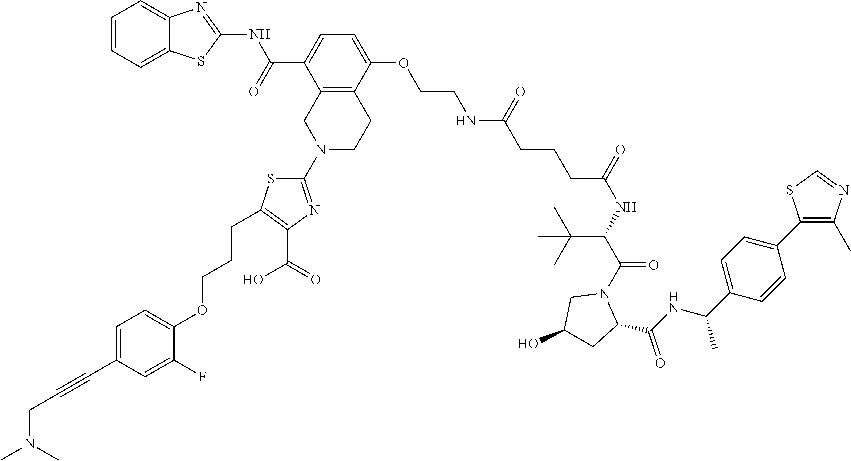 |
| I-50 | 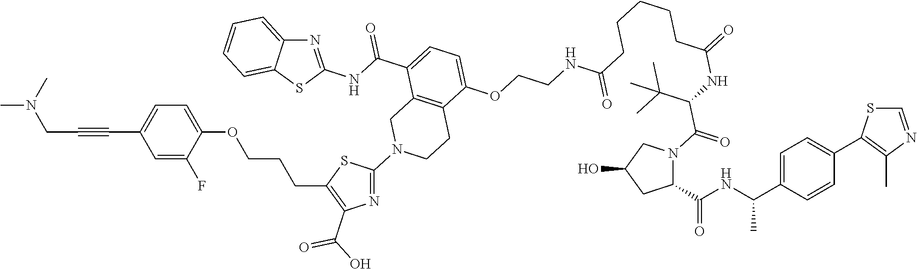 [SMILES: Cc1ncsc1-c1ccc([C@H](C)NC(=O)[C@@H]2C[C@@H](O)CN2C(=O)[C@@H](NC(=O)CCCCCC(=O)NCCOc2ccc(C(=O)Nc3nc4ccccc4s3)c3c2CCN(c2nc(C(=O)O)c(CCCOc4ccc(C#CCN(C)C)cc4F)s2)C3)C(C)(C)C)cc1] |
| I-51 | 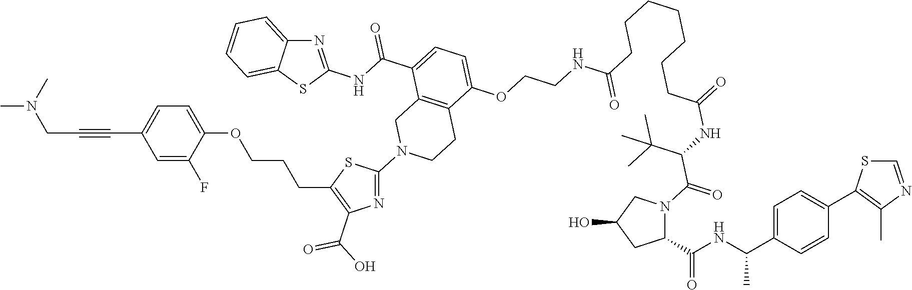 [SMILES: Cc1ncsc1-c1ccc([C@H](C)NC(=O)[C@@H]2C[C@@H](O)CN2C(=O)[C@@H](NC(=O)CCCCCCCC(=O)NCCOc2ccc(C(=O)Nc3nc4ccccc4s3)c3c2CCN(c2nc(C(=O)O)c(CCCOc4ccc(C#CCN(C)C)cc4F)s2)C3)C(C)(C)C)cc1] |
| I-52 | 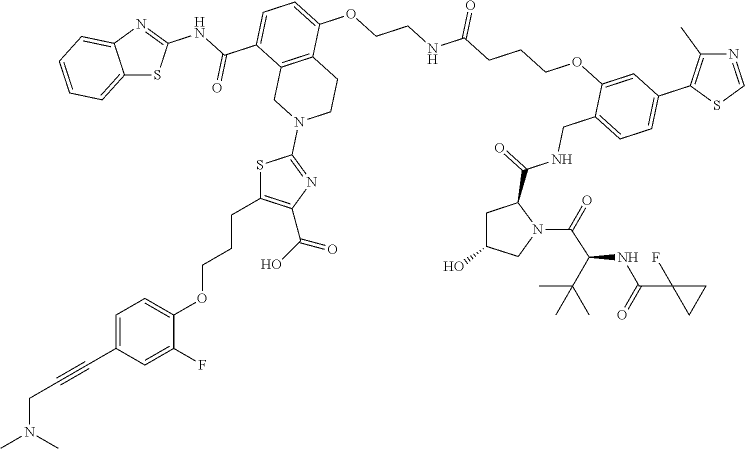 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)NCCOc2ccc(C(=O)Nc3nc4ccccc4s3)c3c2CCN(c2nc(C(=O)O)c(CCCOc4ccc(C#CCN(C)C)cc4F)s2)C3)c1] |
| I-53 | 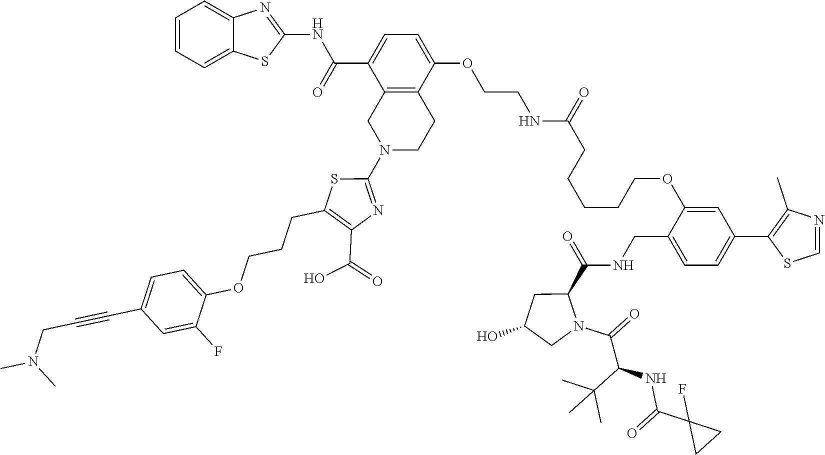 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)NCCOc2ccc(C(=O)Nc3nc4ccccc4s3)c3c2CCN(c2nc(C(=O)O)c(CCCOc4ccc(C#CCN(C)C)cc4F)s2)C3)c1] |
| I-54 | 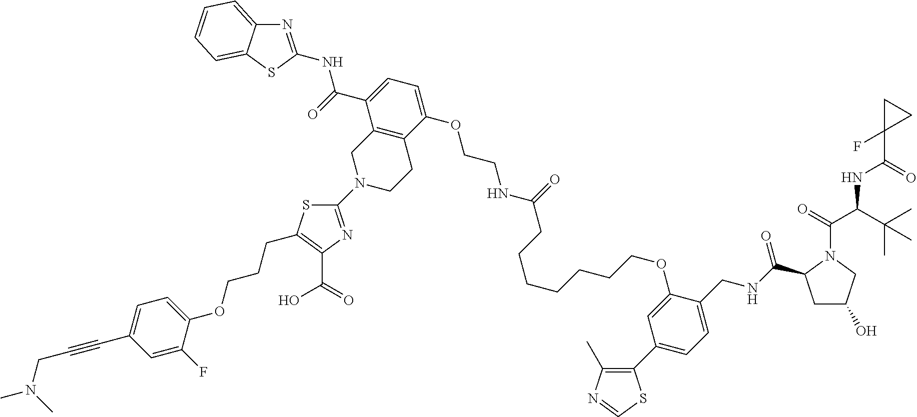 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)NCCOc2ccc(C(=O)Nc3nc4ccccc4s3)c3c2CCN(c2nc(C(=O)O)c(CCCOc4ccc(C#CCN(C)C)cc4F)s2)C3)c1] |
| I-55 | 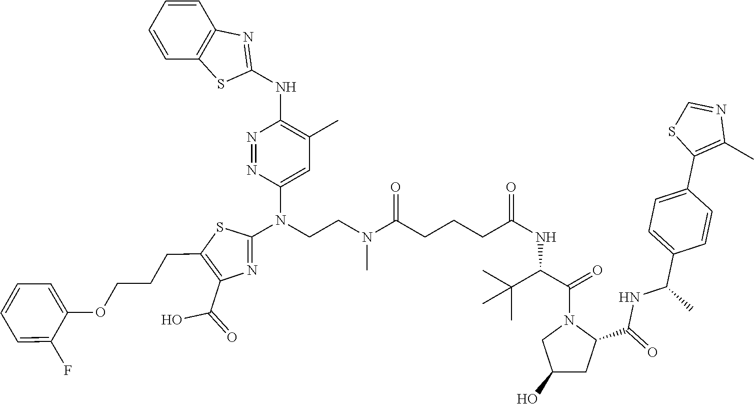 [SMILES: Cc1cc(N(CCN(C)C(=O)CCCC(=O)N[C@H](C(=O)N2C[C@H](O)C[C@H]2C(=O)N[C@@H](C)c2ccc(-c3scnc3C)cc2)C(C)(C)C)c2nc(C(=O)O)c(CCCOc3ccccc3F)s2)nnc1Nc1nc2ccccc2s1] |
| I-56 | 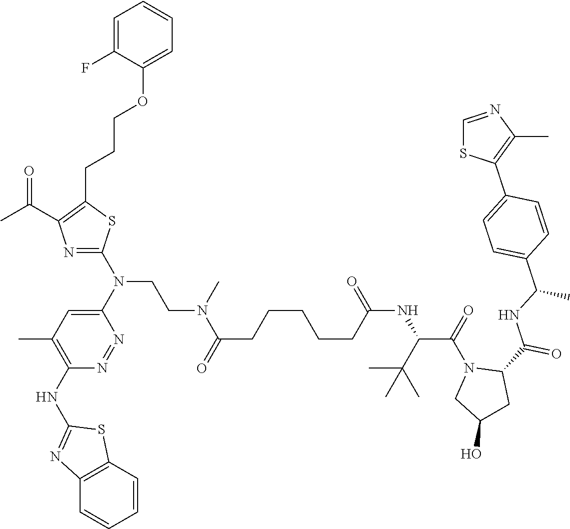 [SMILES: CC(=O)c1nc(N(CCN(C)C(=O)CCCCCC(=O)N[C@H](C(=O)N2C[C@H](O)C[C@H]2C(=O)N[C@@H](C)c2ccc(-c3scnc3C)cc2)C(C)(C)C)c2cc(C)c(Nc3nc4ccccc4s3)nn2)sc1CCCOc1ccccc1F] |
| I-57 | 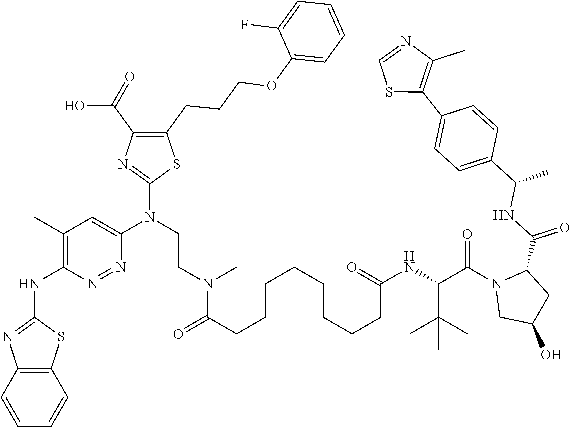 [SMILES: Cc1cc(N(CCN(C)C(=O)CCCCCCCCC(=O)N[C@H](C(=O)N2C[C@H](O)C[C@H]2C(=O)N[C@@H](C)c2ccc(-c3scnc3C)cc2)C(C)(C)C)c2nc(C(=O)O)c(CCCOc3ccccc3F)s2)nnc1Nc1nc2ccccc2s1] |
| I-58 | 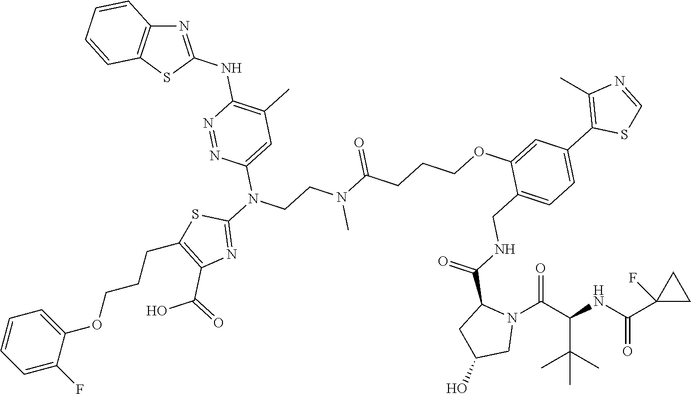 [SMILES: Cc1cc(N(CCN(C)C(=O)CCCOc2cc(-c3scnc3C)ccc2CNC(=O)[C@@H]2C[C@@H](O)CN2C(=O)[C@@H](NC(=O)C2(F)CC2)C(C)(C)C)c2nc(C(=O)O)c(CCCOc3ccccc3F)s2)nnc1Nc1nc2ccccc2s1] |
| I-59 | 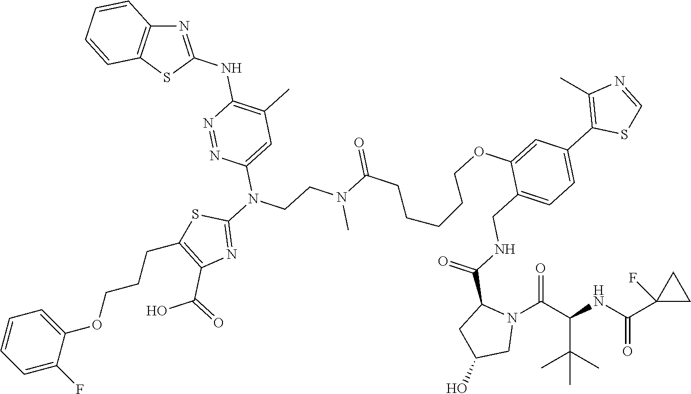 [SMILES: Cc1cc(N(CCN(C)C(=O)CCCCCOc2cc(-c3scnc3C)ccc2CNC(=O)[C@@H]2C[C@@H](O)CN2C(=O)[C@@H](NC(=O)C2(F)CC2)C(C)(C)C)c2nc(C(=O)O)c(CCCOc3ccccc3F)s2)nnc1Nc1nc2ccccc2s1] |
| I-60 | 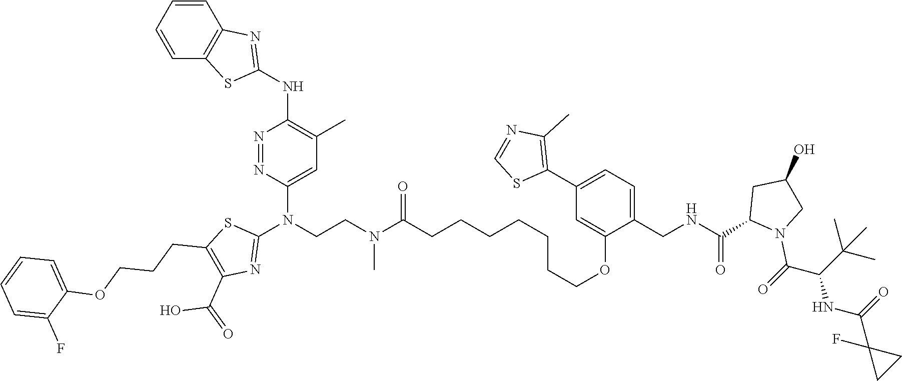 [SMILES: Cc1cc(N(CCN(C)C(=O)CCCCCCCOc2cc(-c3scnc3C)ccc2CNC(=O)[C@@H]2C[C@@H](O)CN2C(=O)[C@@H](NC(=O)C2(F)CC2)C(C)(C)C)c2nc(C(=O)O)c(CCCOc3ccccc3F)s2)nnc1Nc1nc2ccccc2s1] |
| I-61 | 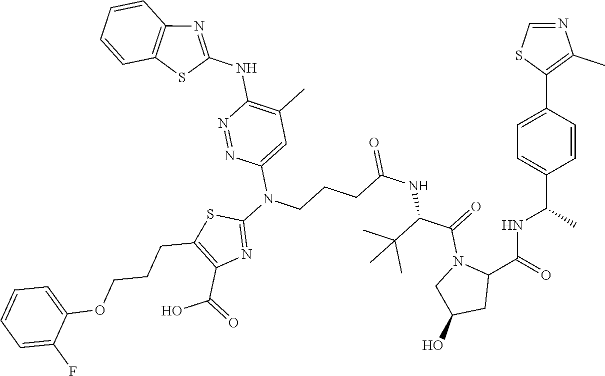 [SMILES: Cc1cc(N(CCCC(=O)N[C@H](C(=O)N2C[C@H](O)CC2C(=O)N[C@@H](C)c2ccc(-c3scnc3C)cc2)C(C)(C)C)c2nc(C(=O)O)c(CCCOc3ccccc3F)s2)nnc1Nc1nc2ccccc2s1] |
| I-62 | 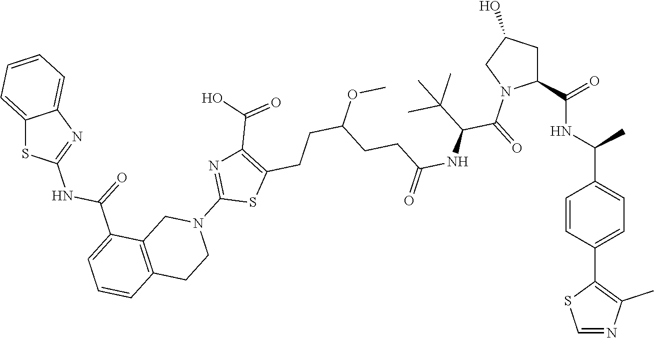 [SMILES: COC(CCC(=O)N[C@H](C(=O)N1C[C@H](O)C[C@H]1C(=O)N[C@@H](C)c1ccc(-c2scnc2C)cc1)C(C)(C)C)CCc1sc(N2CCc3cccc(C(=O)Nc4nc5ccccc5s4)c3C2)nc1C(=O)O] |
| I-63 | 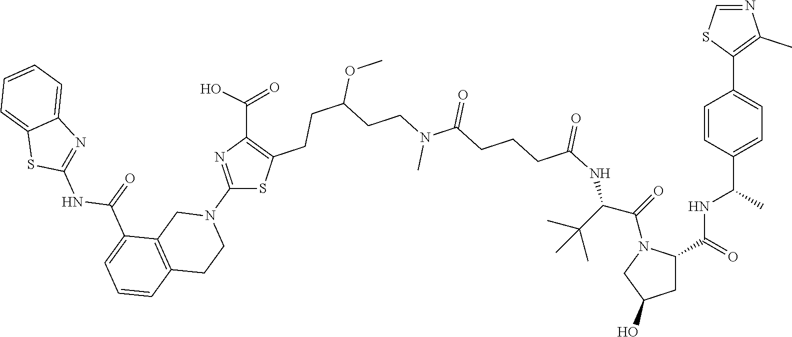 [SMILES: COC(CCc1sc(N2CCc3cccc(C(=O)Nc4nc5ccccc5s4)c3C2)nc1C(=O)O)CCN(C)C(=O)CCCC(=O)N[C@H](C(=O)N1C[C@H](O)C[C@H]1C(=O)N[C@@H](C)c1ccc(-c2scnc2C)cc1)C(C)(C)C] |
| I-64 | 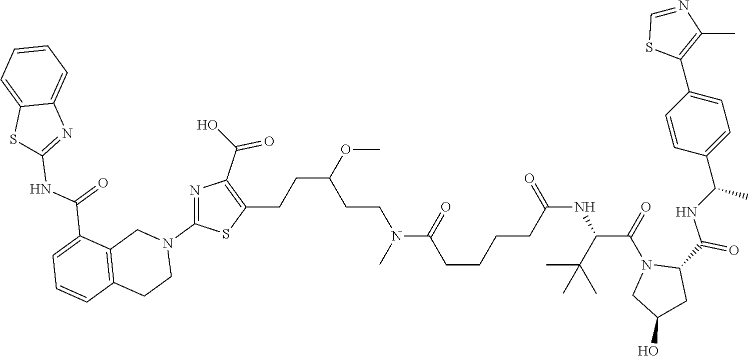 [SMILES: COC(CCc1sc(N2CCc3cccc(C(=O)Nc4nc5ccccc5s4)c3C2)nc1C(=O)O)CCN(C)C(=O)CCCCC(=O)N[C@H](C(=O)N1C[C@H](O)C[C@H]1C(=O)N[C@@H](C)c1ccc(-c2scnc2C)cc1)C(C)(C)C] |
| I-65 | 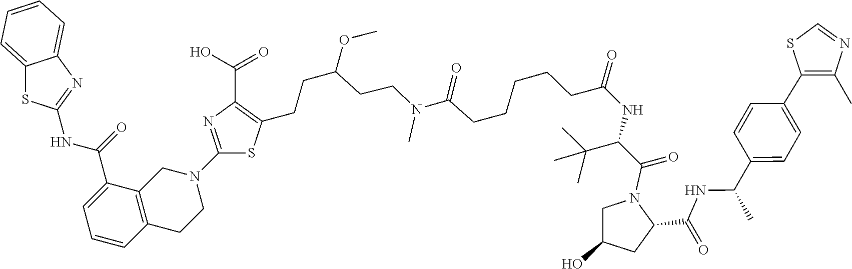 [SMILES: COC(CCc1sc(N2CCc3cccc(C(=O)Nc4nc5ccccc5s4)c3C2)nc1C(=O)O)CCN(C)C(=O)CCCCCC(=O)N[C@H](C(=O)N1C[C@H](O)C[C@H]1C(=O)N[C@@H](C)c1ccc(-c2scnc2C)cc1)C(C)(C)C] |
| I-66 | 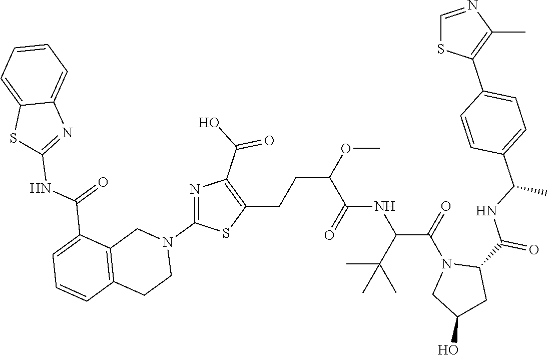 [SMILES: COC(CCc1sc(N2CCc3cccc(C(=O)Nc4nc5ccccc5s4)c3C2)nc1C(=O)O)C(=O)NC(C(=O)N1C[C@H](O)C[C@H]1C(=O)N[C@@H](C)c1ccc(-c2scnc2C)cc1)C(C)(C)C] |
| I-67 | 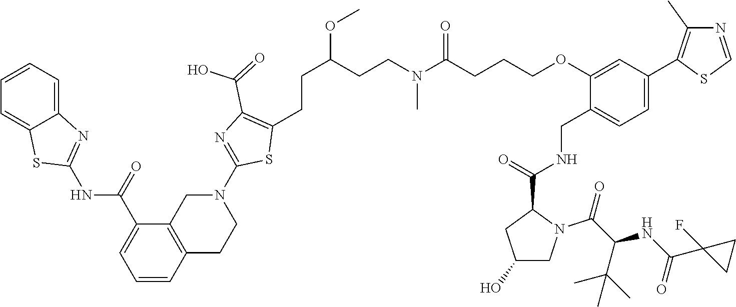 [SMILES: COC(CCc1sc(N2CCc3cccc(C(=O)Nc4nc5ccccc5s4)c3C2)nc1C(=O)O)CCN(C)C(=O)CCCOc1cc(-c2scnc2C)ccc1CNC(=O)[C@@H]1C[C@@H](O)CN1C(=O)[C@@H](NC(=O)C1(F)CC1)C(C)(C)C] |
| I-68 | 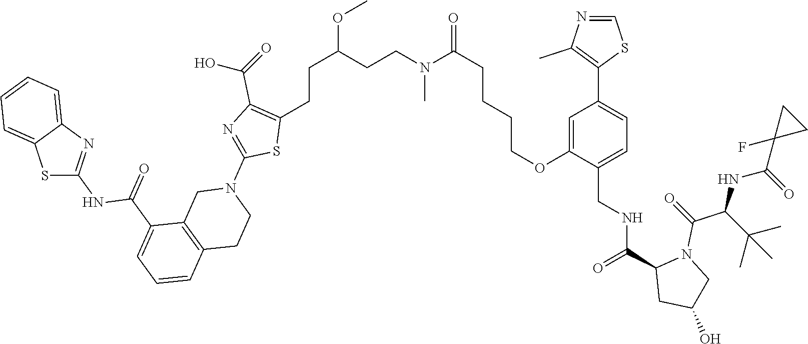 [SMILES: COC(CCc1sc(N2CCc3cccc(C(=O)Nc4nc5ccccc5s4)c3C2)nc1C(=O)O)CCN(C)C(=O)CCCCOc1cc(-c2scnc2C)ccc1CNC(=O)[C@@H]1C[C@@H](O)CN1C(=O)[C@@H](NC(=O)C1(F)CC1)C(C)(C)C] |
| I-69 | 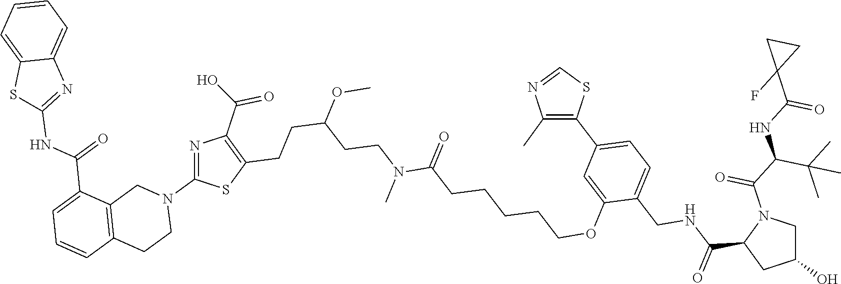 [SMILES: COC(CCc1sc(N2CCc3cccc(C(=O)Nc4nc5ccccc5s4)c3C2)nc1C(=O)O)CCN(C)C(=O)CCCCCOc1cc(-c2scnc2C)ccc1CNC(=O)[C@@H]1C[C@@H](O)CN1C(=O)[C@@H](NC(=O)C1(F)CC1)C(C)(C)C] |
| I-70 | 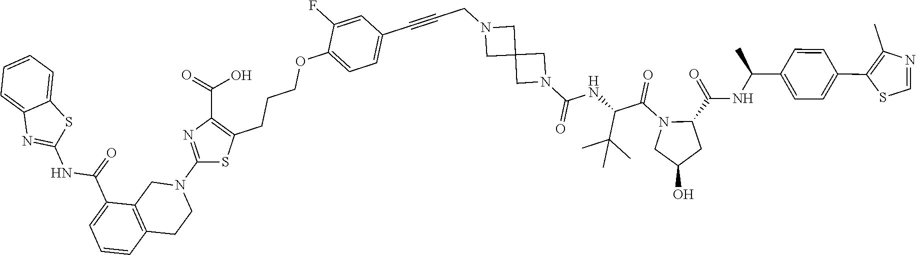 [SMILES: Cc1ncsc1-c1ccc([C@H](C)NC(=O)[C@@H]2C[C@@H](O)CN2C(=O)[C@@H](NC(=O)N2CC3(CN(CC#Cc4ccc(OCCCc5sc(N6CCc7cccc(C(=O)Nc8nc9ccccc9s8)c7C6)nc5C(=O)O)c(F)c4)C3)C2)C(C)(C)C)cc1] |
| I-71 | 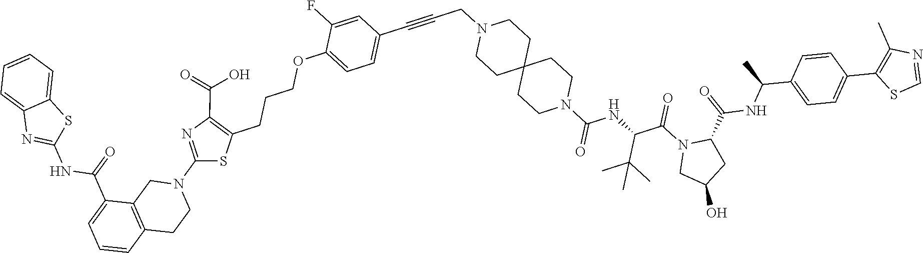 [SMILES: Cc1ncsc1-c1ccc([C@H](C)NC(=O)[C@@H]2C[C@@H](O)CN2C(=O)[C@@H](NC(=O)N2CCC3(CCN(CC#Cc4ccc(OCCCc5sc(N6CCc7cccc(C(=O)Nc8nc9ccccc9s8)c7C6)nc5C(=O)O)c(F)c4)CC3)CC2)C(C)(C)C)cc1] |
| I-72 | 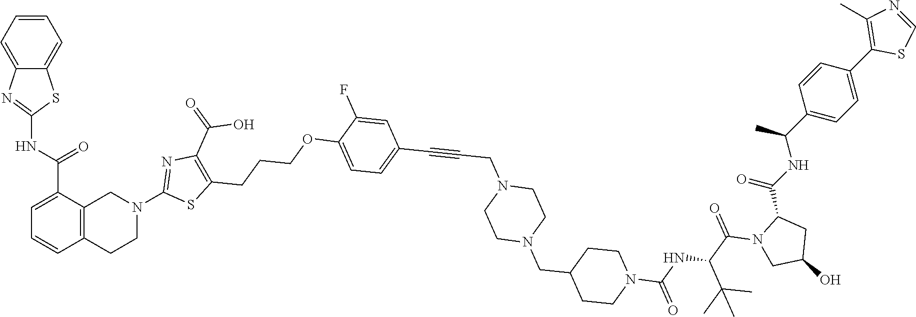 [SMILES: Cc1ncsc1-c1ccc([C@H](C)NC(=O)[C@@H]2C[C@@H](O)CN2C(=O)[C@@H](NC(=O)N2CCC(CN3CCN(CC#Cc4ccc(OCCCc5sc(N6CCc7cccc(C(=O)Nc8nc9ccccc9s8)c7C6)nc5C(=O)O)c(F)c4)CC3)CC2)C(C)(C)C)cc1] |
| I-73 | 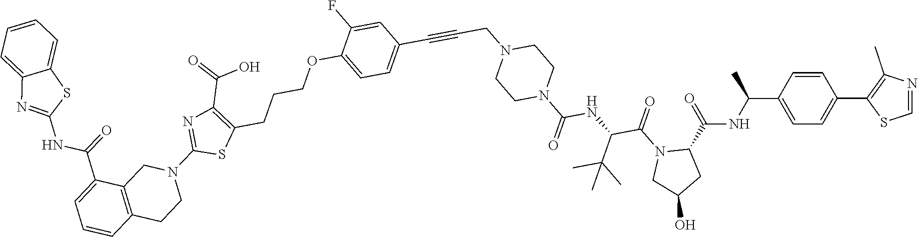 [SMILES: Cc1ncsc1-c1ccc([C@H](C)NC(=O)[C@@H]2C[C@@H](O)CN2C(=O)[C@@H](NC(=O)N2CCN(CC#Cc3ccc(OCCCc4sc(N5CCc6cccc(C(=O)Nc7nc8ccccc8s7)c6C5)nc4C(=O)O)c(F)c3)CC2)C(C)(C)C)cc1] |
| I-74 | 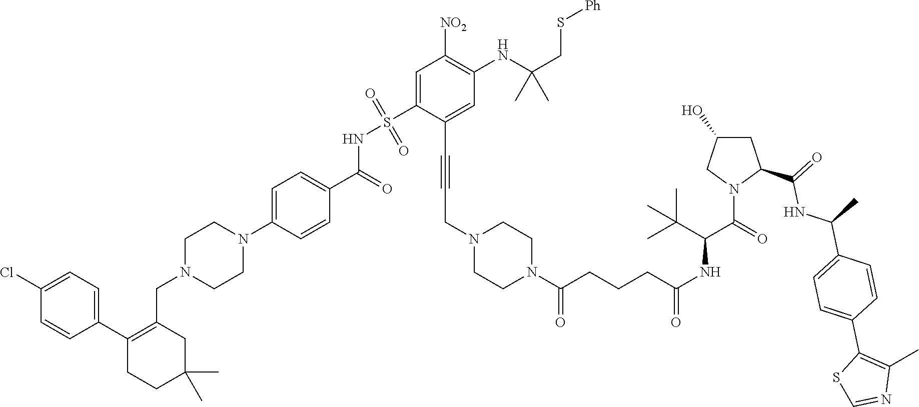 [SMILES: Cc1ncsc1-c1ccc([C@H](C)NC(=O)[C@@H]2C[C@@H](O)CN2C(=O)[C@@H](NC(=O)CCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)C(C)(C)C)cc1] |
| I-75 | 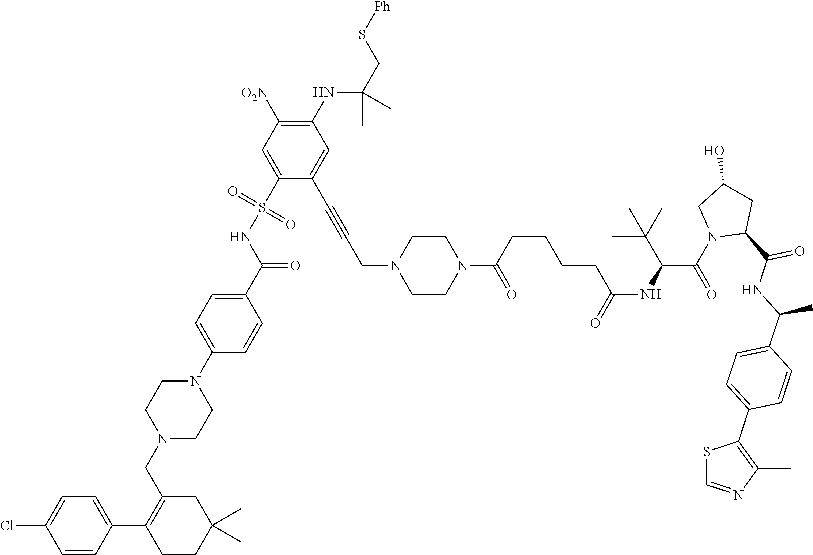 [SMILES: Cc1ncsc1-c1ccc([C@H](C)NC(=O)[C@@H]2C[C@@H](O)CN2C(=O)[C@@H](NC(=O)CCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)C(C)(C)C)cc1] |
| I-76 | 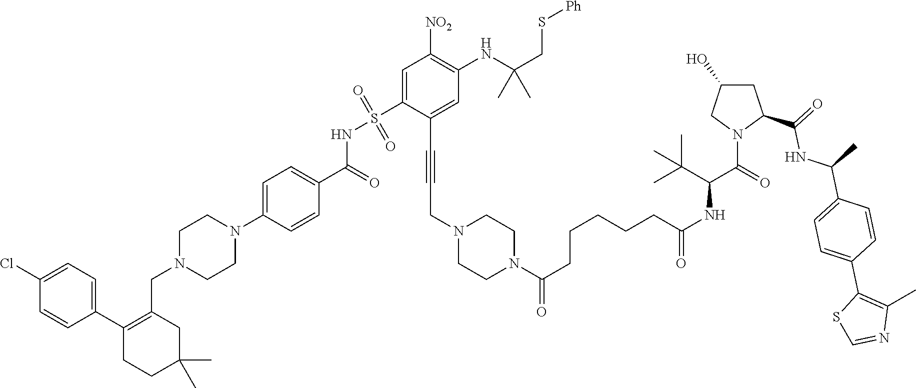 [SMILES: Cc1ncsc1-c1ccc([C@H](C)NC(=O)[C@@H]2C[C@@H](O)CN2C(=O)[C@@H](NC(=O)CCCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)C(C)(C)C)cc1] |
| I-77 | 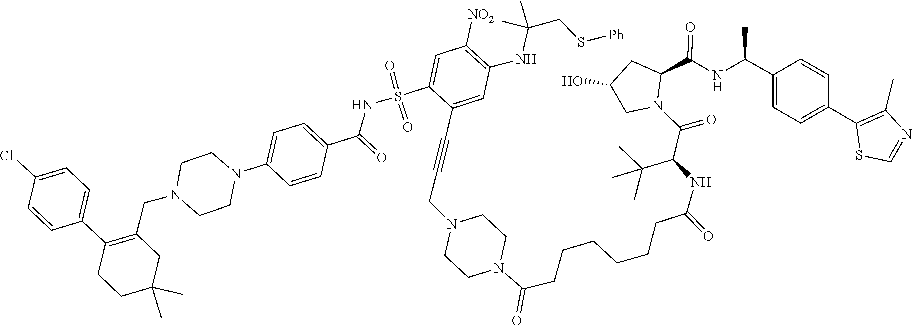 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)C(C)(C)C)cc1] |
| I-78 | 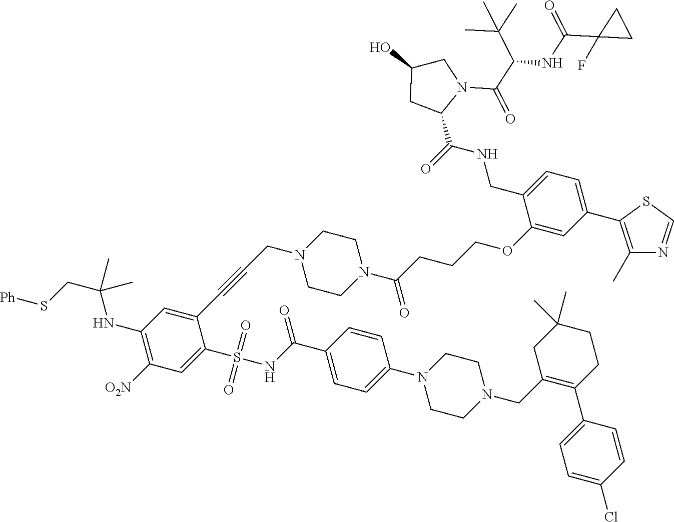 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)c1] |
| I-79 | 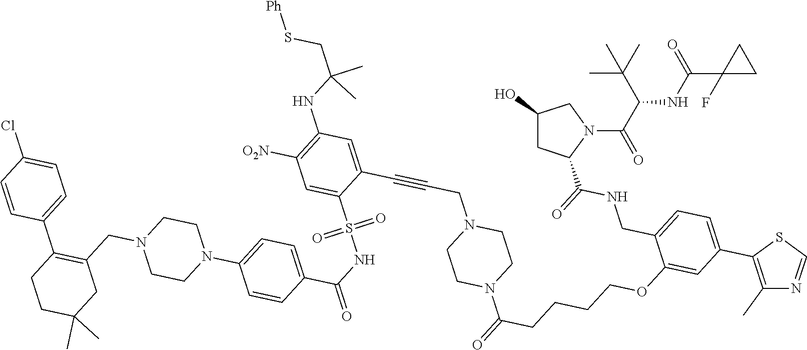 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)c1] |
| I-80 | 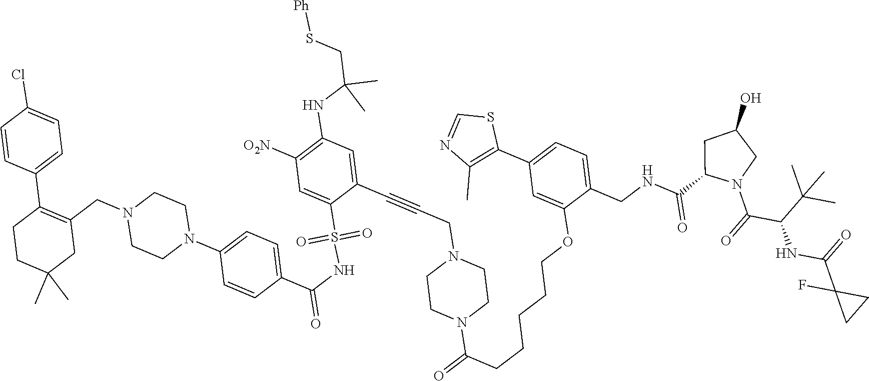 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)c1] |
| I-81 | 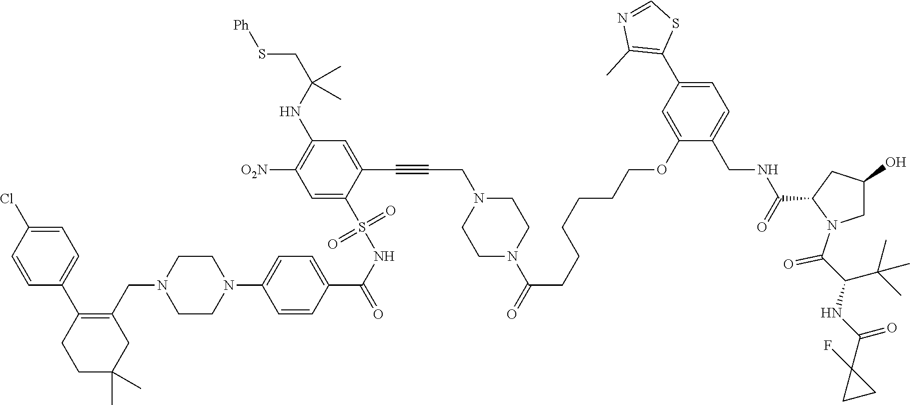 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)c1] |
| I-82 | 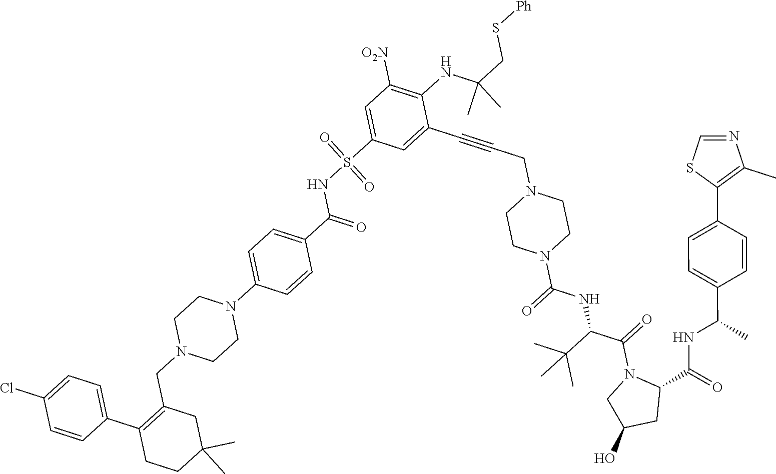 [SMILES: Cc1ncsc1-c1ccc([C@H](C)NC(=O)[C@@H]2C[C@@H](O)CN2C(=O)[C@@H](NC(=O)N2CCN(CC#Cc3cc(S(=O)(=O)NC(=O)c4ccc(N5CCN(CC6=C(c7ccc(Cl)cc7)CCC(C)(C)C6)CC5)cc4)cc([N+](=O)[O-])c3NC(C)(C)CSc3ccccc3)CC2)C(C)(C)C)cc1] |
| I-83 | 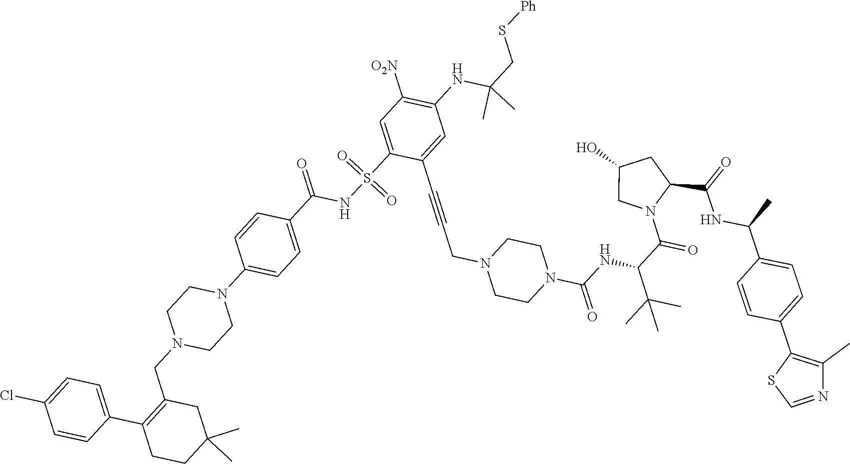 [SMILES: Cc1ncsc1-c1ccc([C@H](C)NC(=O)[C@@H]2C[C@@H](O)CN2C(=O)[C@@H](NC(=O)N2CCN(CC#Cc3cc(NC(C)(C)CSc4ccccc4)c([N+](=O)[O-])cc3S(=O)(=O)NC(=O)c3ccc(N4CCN(CC5=C(c6ccc(Cl)cc6)CCC(C)(C)C5)CC4)cc3)CC2)C(C)(C)C)cc1] |
| I-84 | 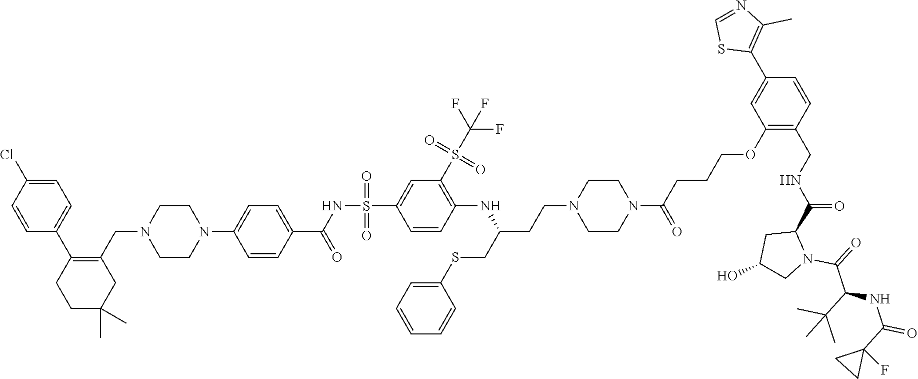 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-85 | 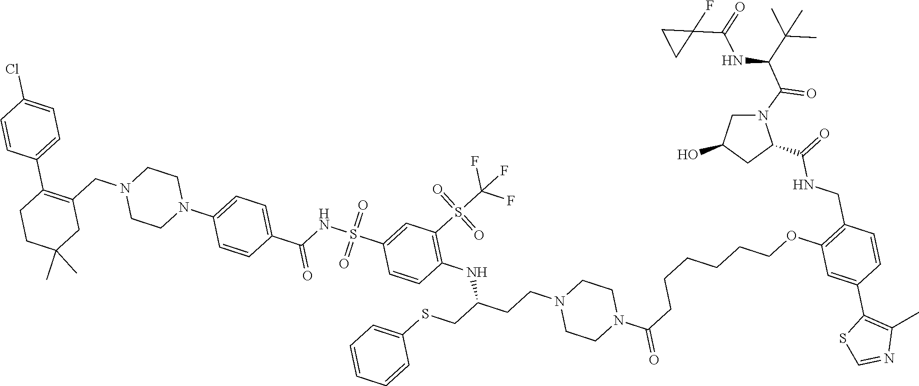 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-86 | 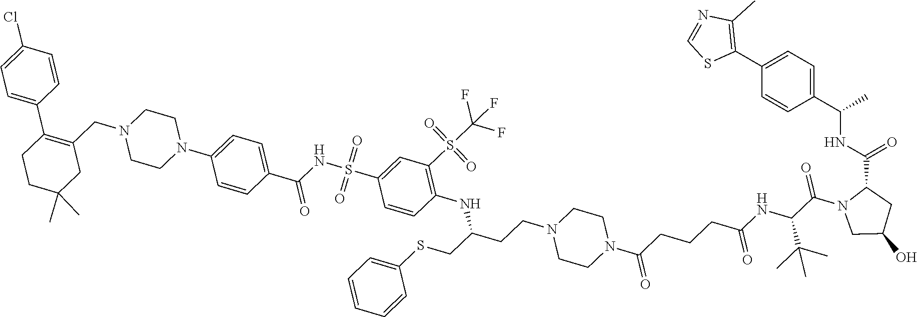 [SMILES: Cc1ncsc1-c1ccc([C@H](C)NC(=O)[C@@H]2C[C@@H](O)CN2C(=O)[C@@H](NC(=O)CCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-87 | 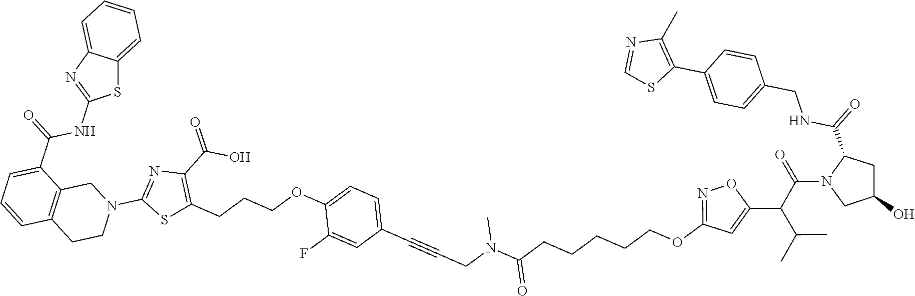 [SMILES: Cc1ncsc1-c1ccc(CNC(=O)[C@@H]2C[C@@H](O)CN2C(=O)C(c2cc(OCCCCCC(=O)N(C)CC#Cc3ccc(OCCCc4sc(N5CCc6cccc(C(=O)Nc7nc8ccccc8s7)c6C5)nc4C(=O)O)c(F)c3)no2)C(C)C)cc1] |
| I-88 | 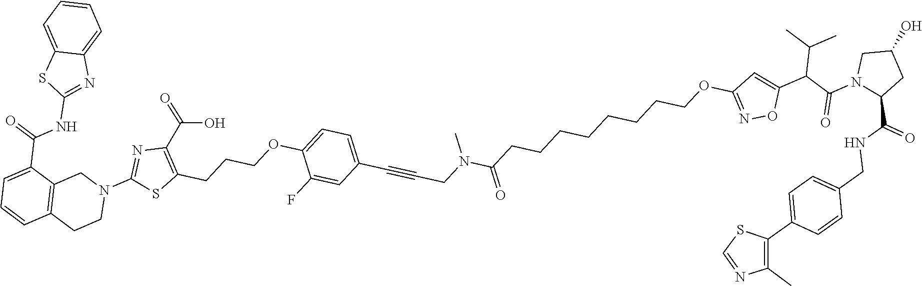 [SMILES: Cc1ncsc1-c1ccc(CNC(=O)[C@@H]2C[C@@H](O)CN2C(=O)C(c2cc(OCCCCCCCCC(=O)N(C)CC#Cc3ccc(OCCCc4sc(N5CCc6cccc(C(=O)Nc7nc8ccccc8s7)c6C5)nc4C(=O)O)c(F)c3)no2)C(C)C)cc1] |
| I-89 | 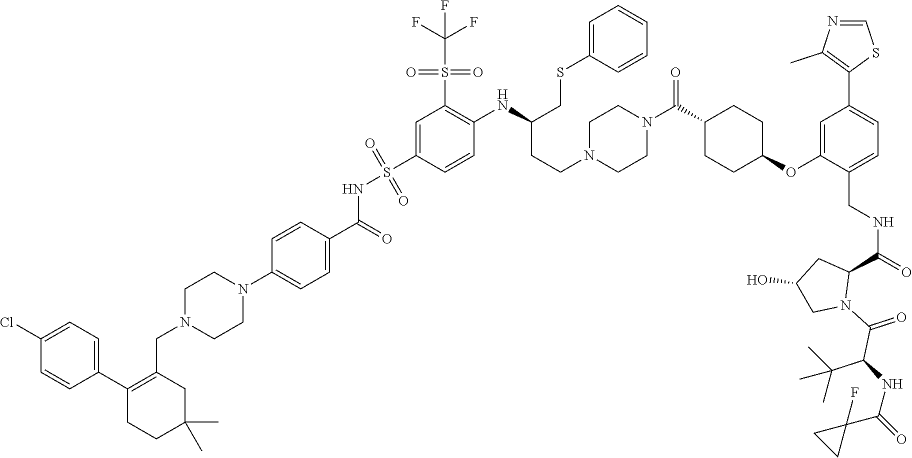 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](C(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-90 | 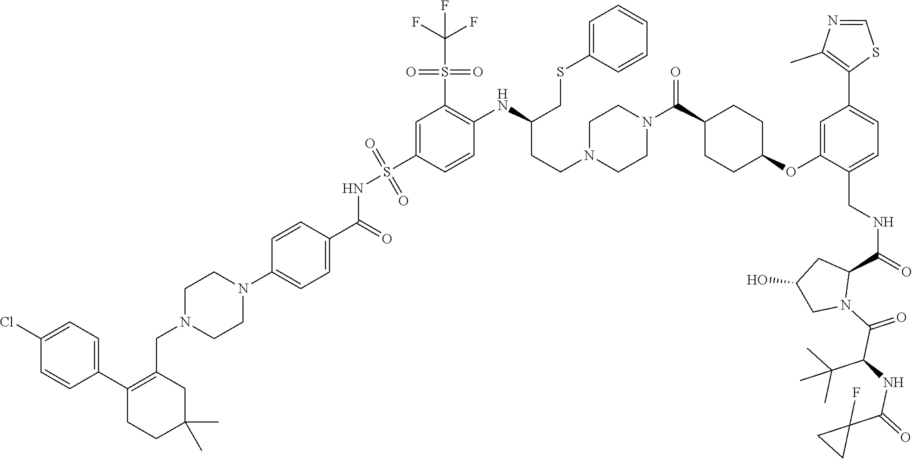 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](C(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-91 | 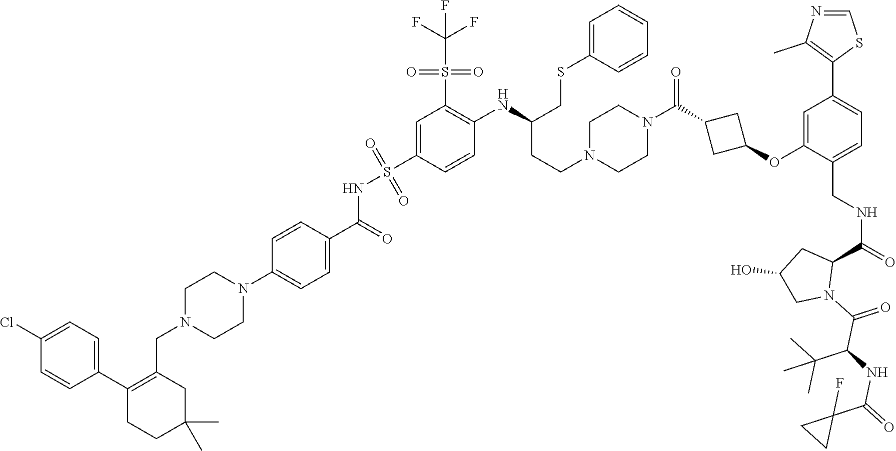 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2C[C@H](C(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)C2)c1] |
| I-92 | 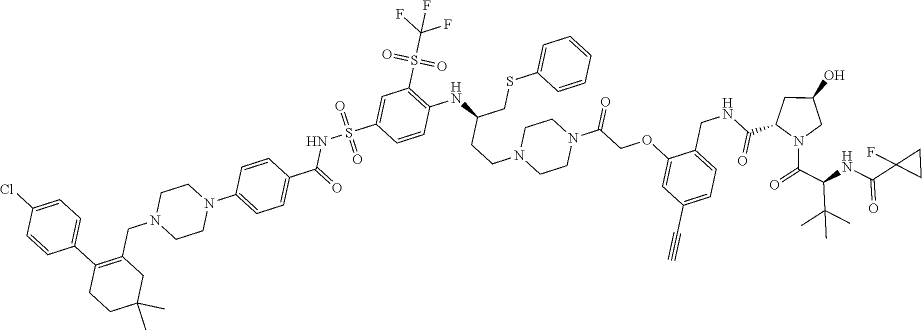 [SMILES: C#Cc1ccc(CNC(=O)[C@@H]2C[C@@H](O)CN2C(=O)[C@@H](NC(=O)C2(F)CC2)C(C)(C)C)c(OCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-93 | 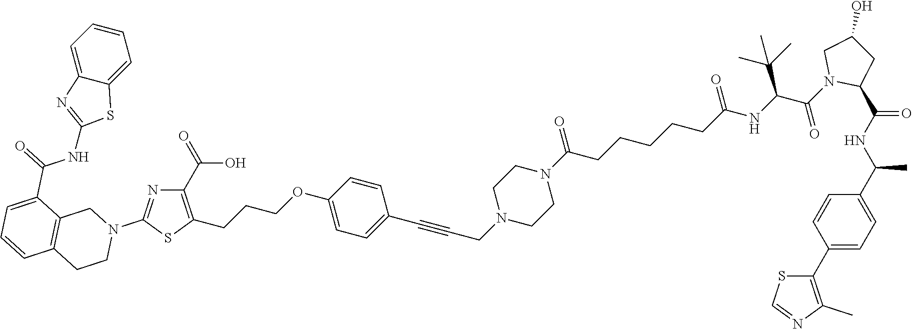 [SMILES: Cc1ncsc1-c1ccc([C@H](C)NC(=O)[C@@H]2C[C@@H](O)CN2C(=O)[C@@H](NC(=O)CCCCCC(=O)N2CCN(CC#Cc3ccc(OCCCc4sc(N5CCc6cccc(C(=O)Nc7nc8ccccc8s7)c6C5)nc4C(=O)O)cc3)CC2)C(C)(C)C)cc1] |
| I-94 | 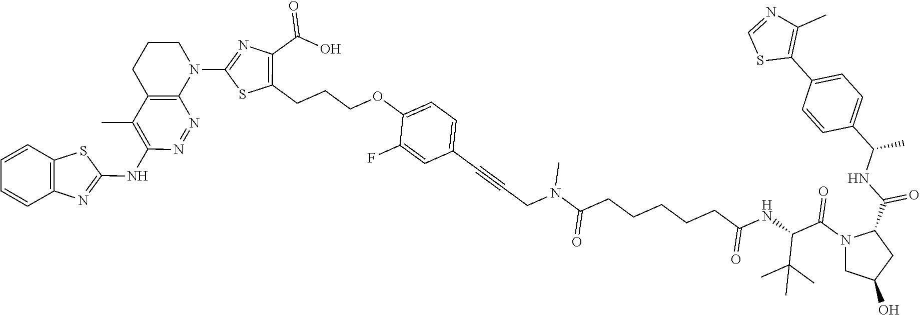 [SMILES: Cc1ncsc1-c1ccc([C@H](C)NC(=O)[C@@H]2C[C@@H](O)CN2C(=O)[C@@H](NC(=O)CCCCCC(=O)N(C)CC#Cc2ccc(OCCCc3sc(N4CCCc5c4nnc(Nc4nc6ccccc6s4)c5C)nc3C(=O)O)c(F)c2)C(C)(C)C)cc1] |
| I-95 | 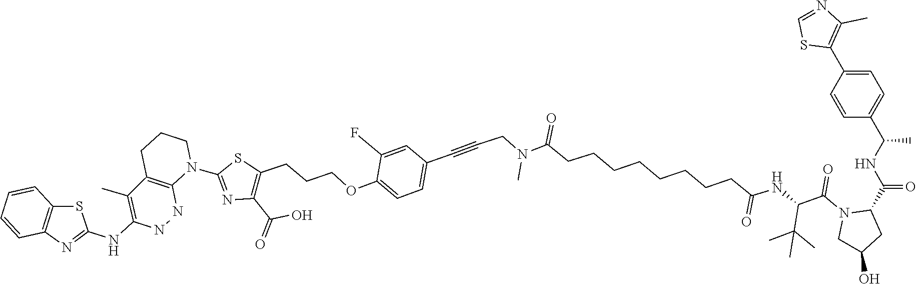 |
| I-96 | 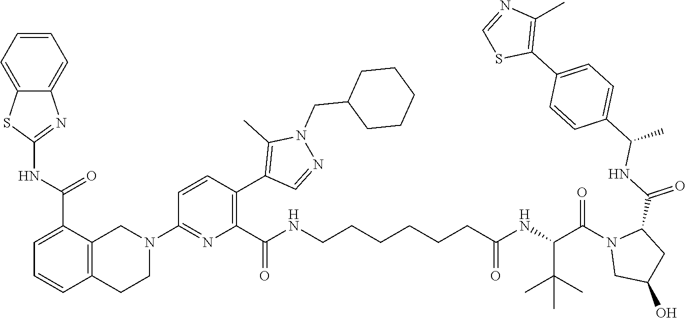 [SMILES: Cc1ncsc1-c1ccc([C@H](C)NC(=O)[C@@H]2C[C@@H](O)CN2C(=O)[C@@H](NC(=O)CCCCCCNC(=O)c2nc(N3CCc4cccc(C(=O)Nc5nc6ccccc6s5)c4C3)ccc2-c2cnn(CC3CCCCC3)c2C)C(C)(C)C)cc1] |
| I-97 | 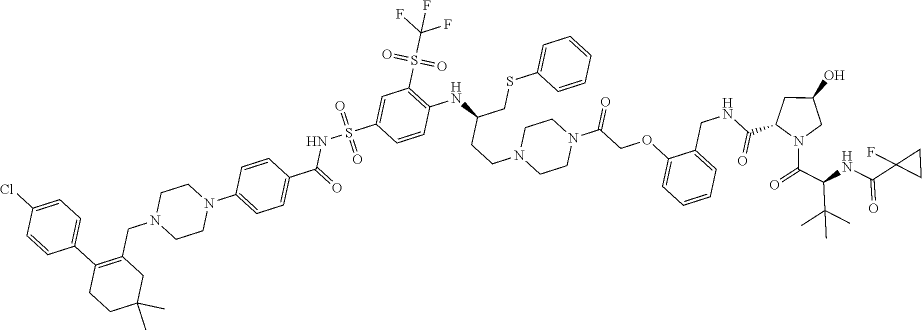 [SMILES: CC1(C)CCC(c2ccc(Cl)cc2)=C(CN2CCN(c3ccc(C(=O)NS(=O)(=O)c4ccc(N[C@H](CCN5CCN(C(=O)COc6ccccc6CNC(=O)[C@@H]6C[C@@H](O)CN6C(=O)[C@@H](NC(=O)C6(F)CC6)C(C)(C)C)CC5)CSc5ccccc5)c(S(=O)(=O)C(F)(F)F)c4)cc3)CC2)C1] |
| I-98 | 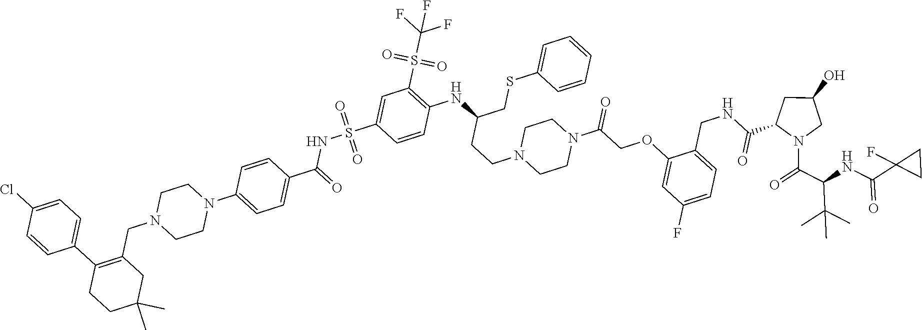 [SMILES: CC1(C)CCC(c2ccc(Cl)cc2)=C(CN2CCN(c3ccc(C(=O)NS(=O)(=O)c4ccc(N[C@H](CCN5CCN(C(=O)COc6cc(F)ccc6CNC(=O)[C@@H]6C[C@@H](O)CN6C(=O)[C@@H](NC(=O)C6(F)CC6)C(C)(C)C)CC5)CSc5ccccc5)c(S(=O)(=O)C(F)(F)F)c4)cc3)CC2)C1] |
| I-99 | 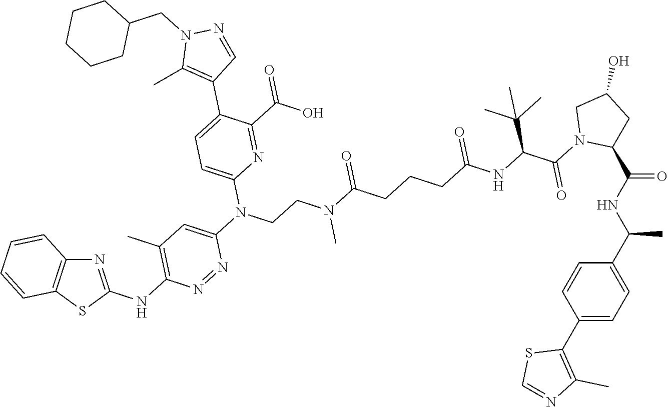 [SMILES: Cc1cc(N(CCN(C)C(=O)CCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC4CCCCC4)c3C)c(C(=O)O)n2)nnc1Nc1nc2ccccc2s1] |
| I-100 | 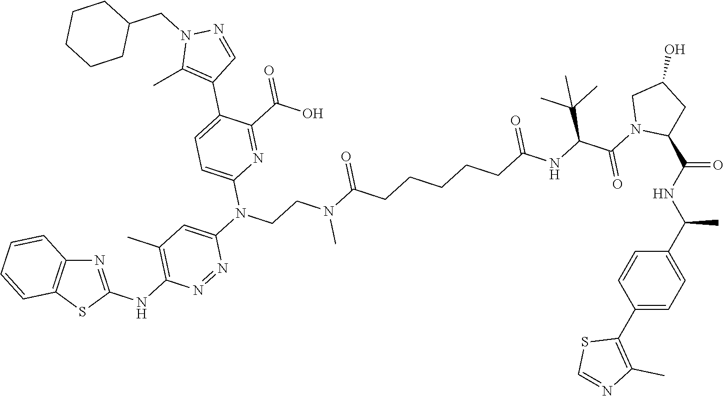 [SMILES: Cc1cc(N(CCN(C)C(=O)CCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC4CCCCC4)c3C)c(C(=O)O)n2)nnc1Nc1nc2ccccc2s1] |
| I-101 | 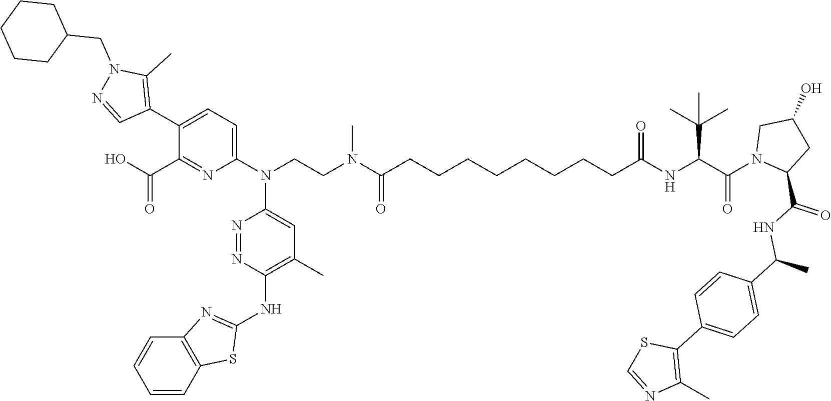 [SMILES: Cc1cc(N(CCN(C)C(=O)CCCCCCCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC4CCCCC4)c3C)c(C(=O)O)n2)nnc1Nc1nc2ccccc2s1] |
| I-102 | 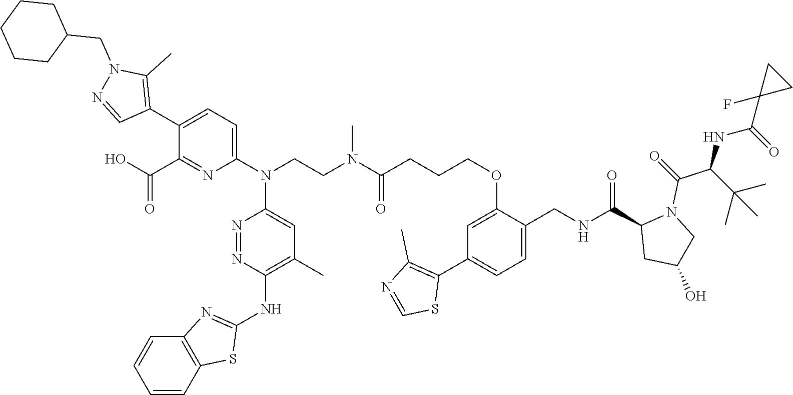 [SMILES: Cc1cc(N(CCN(C)C(=O)CCCOc2cc(-c3scnc3C)ccc2CNC(=O)[C@@H]2C[C@@H](O)CN2C(=O)[C@@H](NC(=O)C2(F)CC2)C(C)(C)C)c2ccc(-c3cnn(CC4CCCCC4)c3C)c(C(=O)O)n2)nnc1Nc1nc2ccccc2s1] |
| I-103 | 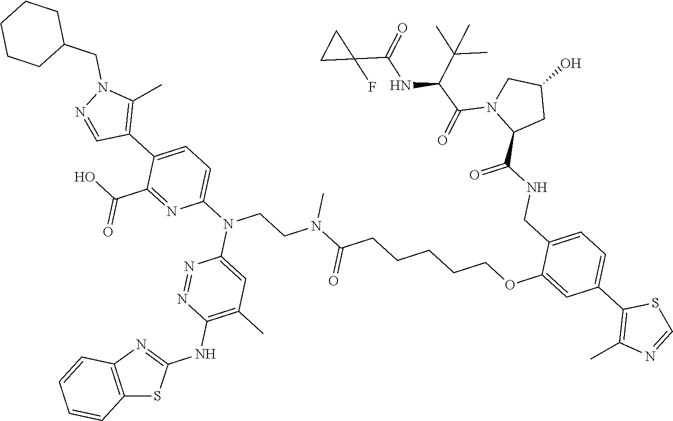 [SMILES: Cc1cc(N(CCN(C)C(=O)CCCCCOc2cc(-c3scnc3C)ccc2CNC(=O)[C@@H]2C[C@@H](O)CN2C(=O)[C@@H](NC(=O)C2(F)CC2)C(C)(C)C)c2ccc(-c3cnn(CC4CCCCC4)c3C)c(C(=O)O)n2)nnc1Nc1nc2ccccc2s1] |
| I-104 | 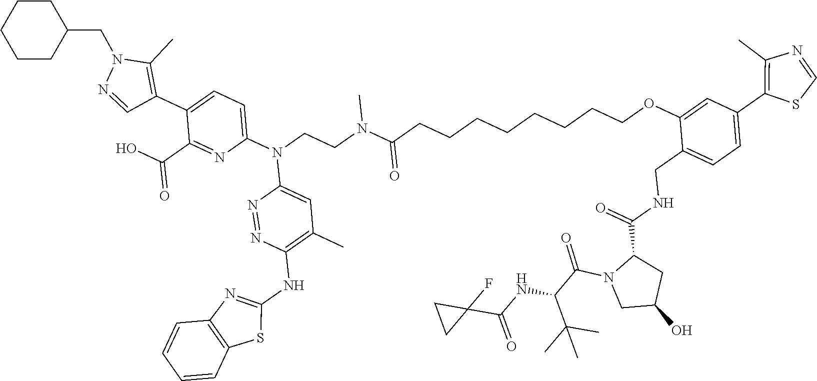 [SMILES: Cc1cc(N(CCN(C)C(=O)CCCCCCCCOc2cc(-c3scnc3C)ccc2CNC(=O)[C@@H]2C[C@@H](O)CN2C(=O)[C@@H](NC(=O)C2(F)CC2)C(C)(C)C)c2ccc(-c3cnn(CC4CCCCC4)c3C)c(C(=O)O)n2)nnc1Nc1nc2ccccc2s1] |
| I-105 | 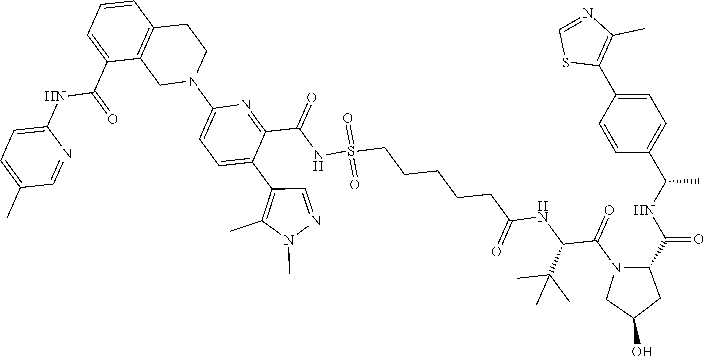 [SMILES: Cc1ccc(NC(=O)c2cccc3c2CN(c2ccc(-c4cnn(C)c4C)c(C(=O)NS(=O)(=O)CCCCCC(=O)N[C@H](C(=O)N4C[C@H](O)C[C@H]4C(=O)N[C@@H](C)c4ccc(-c5scnc5C)cc4)C(C)(C)C)n2)CC3)nc1] |
| I-106 | 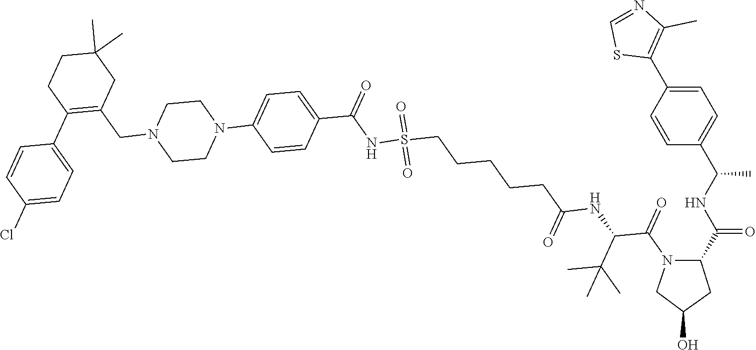 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2ccc(N3CCN(CC4=C(c5ccc(Cl)cc5)CCC(C)(C)C4)CC3)cc2)C(C)(C)C)cc1] |
| I-107 | 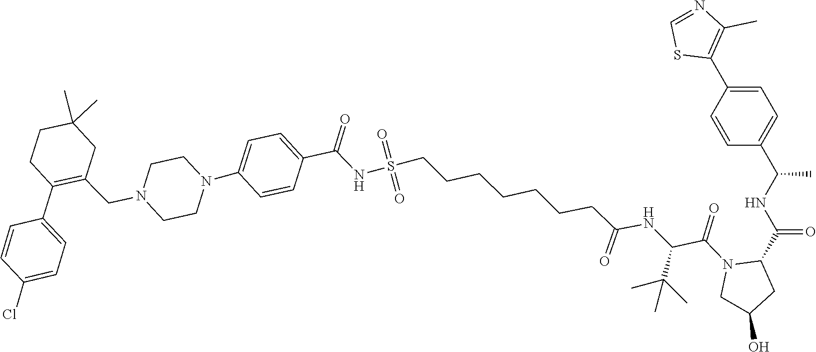 [SMILES: Cc1ncsc1-c1ccc([C@H](C)NC(=O)[C@@H]2C[C@@H](O)CN2C(=O)[C@@H](NC(=O)CCCCCCCS(=O)(=O)NC(=O)c2ccc(N3CCN(CC4=C(c5ccc(Cl)cc5)CCC(C)(C)C4)CC3)cc2)C(C)(C)C)cc1] |
| I-108 | 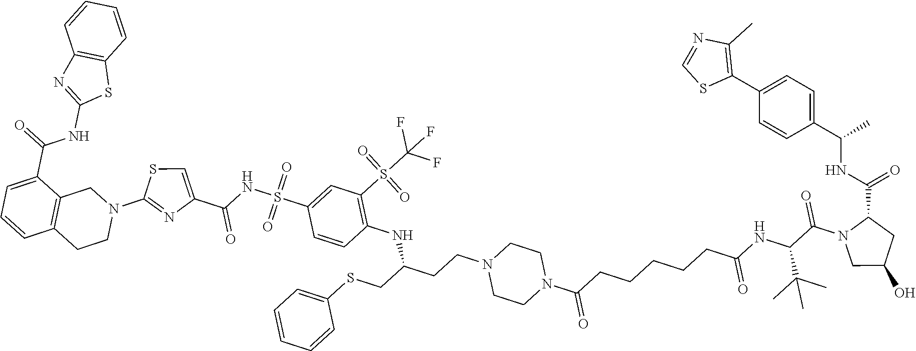 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4csc(N5CCc6cccc(C(=O)Nc7nc8ccccc8s7)c6C5)n4)cc3S(=O)(=O)C(F)(F)F)CC2)C(C)(C)C)cc1] |
| I-109 | 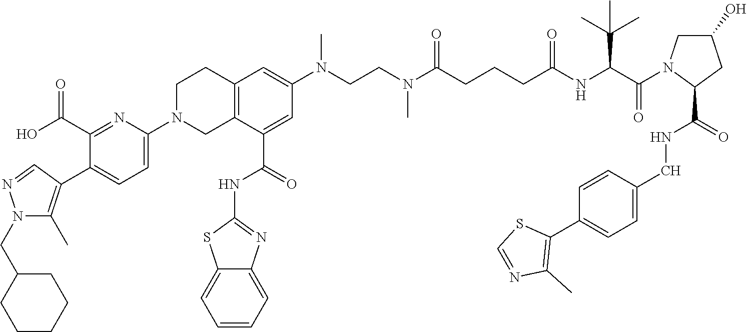 [SMILES: Cc1ncsc1-c1ccc(CNC(=O)[C@@H]2C[C@@H](O)CN2C(=O)[C@@H](NC(=O)CCCC(=O)N(C)CCN(C)c2cc3c(c(C(=O)Nc4nc5ccccc5s4)c2)CN(c2ccc(-c4cnn(CC5CCCCC5)c4C)c(C(=O)O)n2)CC3)C(C)(C)C)cc1] |
| I-110 | 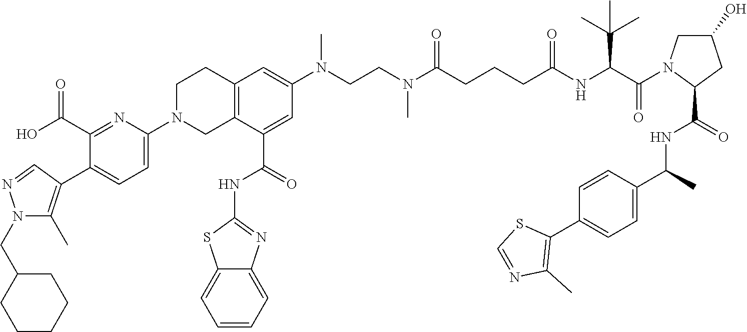 [SMILES: Cc1ncsc1-c1ccc([C@H](C)NC(=O)[C@@H]2C[C@@H](O)CN2C(=O)[C@@H](NC(=O)CCCC(=O)N(C)CCN(C)c2cc3c(c(C(=O)Nc4nc5ccccc5s4)c2)CN(c2ccc(-c4cnn(CC5CCCCC5)c4C)c(C(=O)O)n2)CC3)C(C)(C)C)cc1] |
| I-111 | 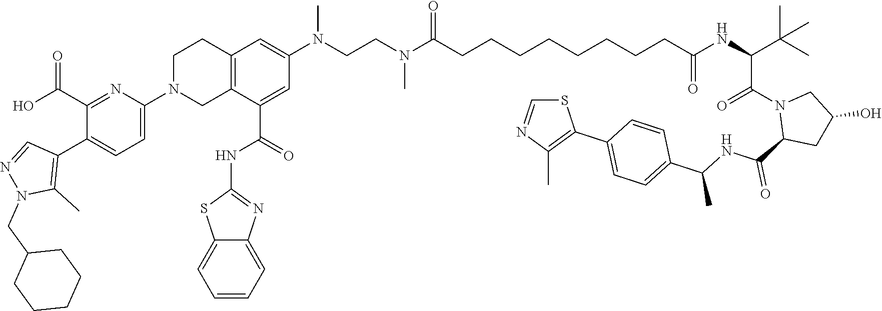 [SMILES: Cc1ncsc1-c1ccc([C@H](C)NC(=O)[C@@H]2C[C@@H](O)CN2C(=O)[C@@H](NC(=O)CCCCCCCCC(=O)N(C)CCN(C)c2cc3c(c(C(=O)Nc4nc5ccccc5s4)c2)CN(c2ccc(-c4cnn(CC5CCCCC5)c4C)c(C(=O)O)n2)CC3)C(C)(C)C)cc1] |
| I-112 | 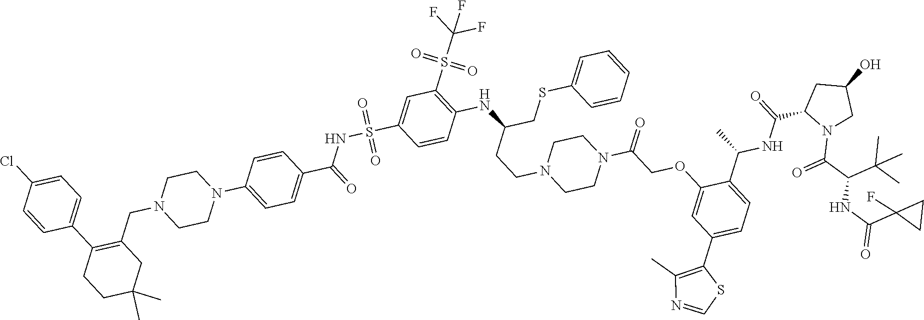 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(OCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-113 | 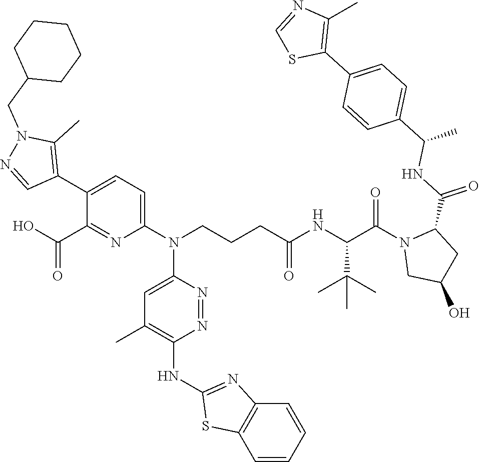 [SMILES: Cc1cc(N(CCCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC4CCCCC4)c3C)c(C(=O)O)n2)nnc1Nc1nc2ccccc2s1] |
| I-114 | 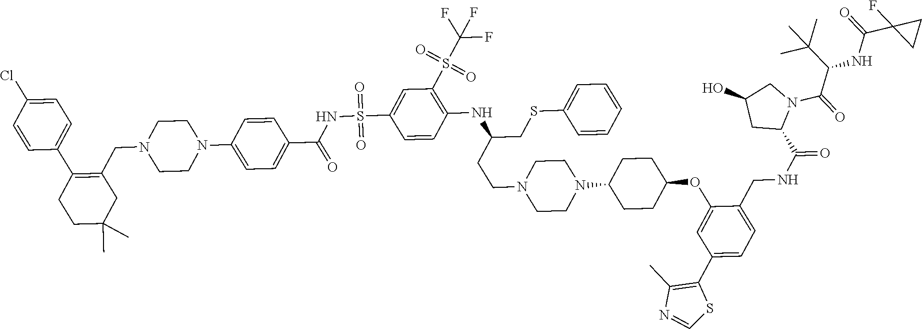 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-115 | 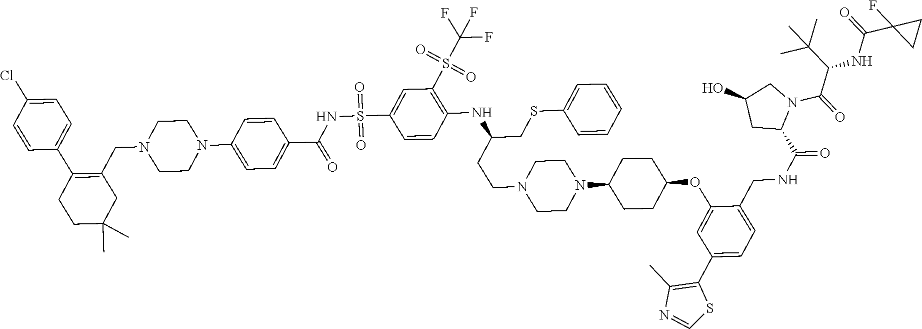 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-116 | 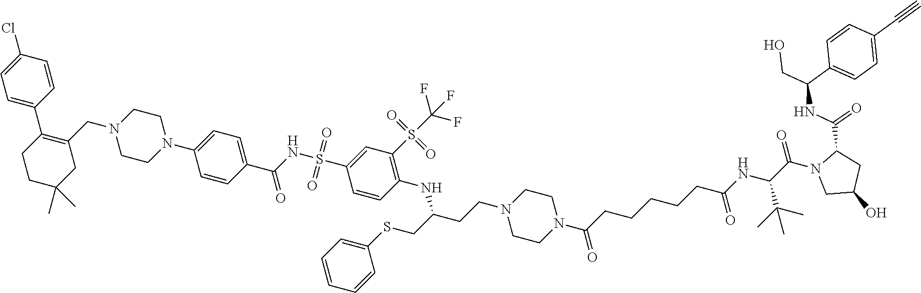 [SMILES: C#Cc1ccc([C@H](CO)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-117 | 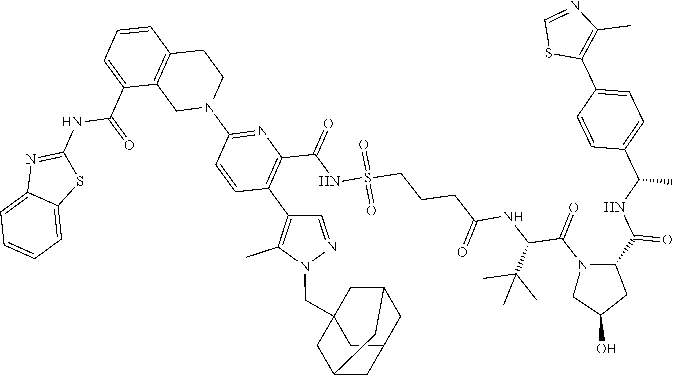 [SMILES: Cc1ncsc1-c1ccc([C@H](C)NC(=O)[C@@H]2C[C@@H](O)CN2C(=O)[C@@H](NC(=O)CCCS(=O)(=O)NC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| I-118 | 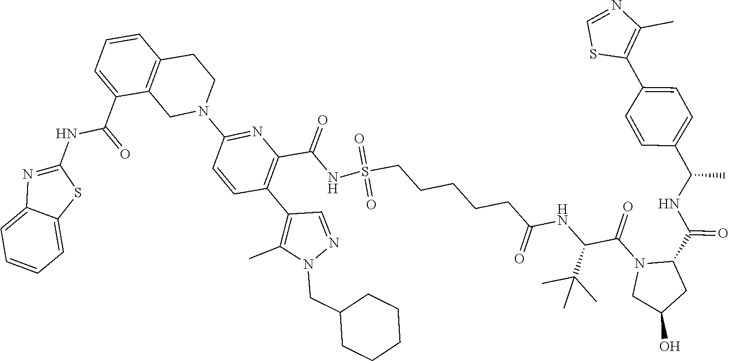 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-119 | 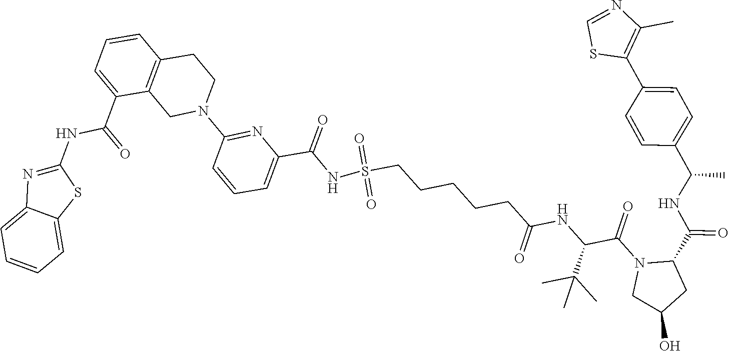 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2cccc(N3CCc4cccc(C(=O)Nc5nc6ccccc6s5)c4C3)n2)C(C)(C)C)cc1] |
| I-120 | 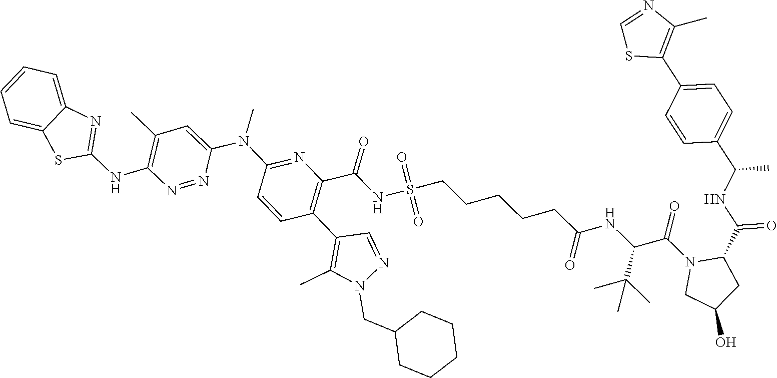 [SMILES: Cc1cc(N(C)c2ccc(-c3cnn(CC4CCCCC4)c3C)c(C(=O)NS(=O)(=O)CCCCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-121 | 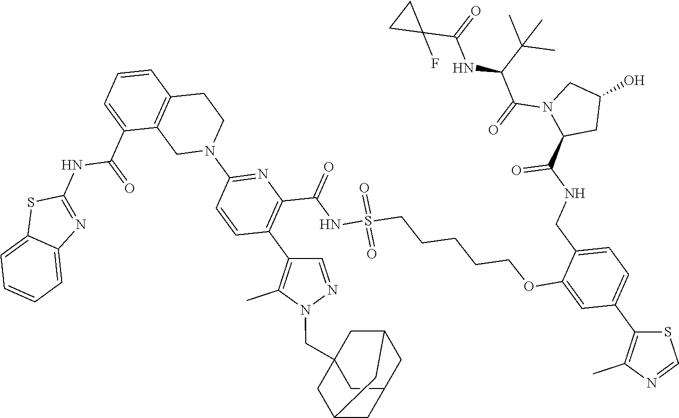 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCS(=O)(=O)NC(=O)c2nc(N3CCc4cccc(C(=O)Nc5nc6ccccc6s5)c4C3)ccc2-c2cnn(CC34CC5CC(CC(C5)C3)C4)c2C)c1] |
| I-122 | 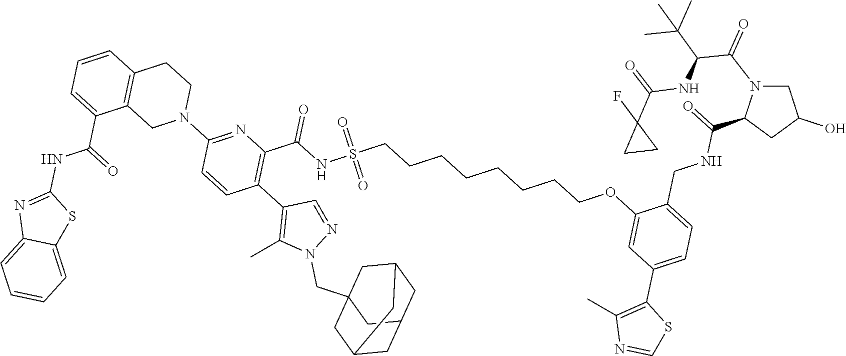 [SMILES: Cc1ncsc1-c1ccc(CNC(=O)[C@@H]2CC(O)CN2C(=O)[C@@H](NC(=O)C2(F)CC2)C(C)(C)C)c(OCCCCCCCCS(=O)(=O)NC(=O)c2nc(N3CCc4cccc(C(=O)Nc5nc6ccccc6s5)c4C3)ccc2-c2cnn(CC34CC5CC(CC(C5)C3)C4)c2C)c1] |
| I-123 | 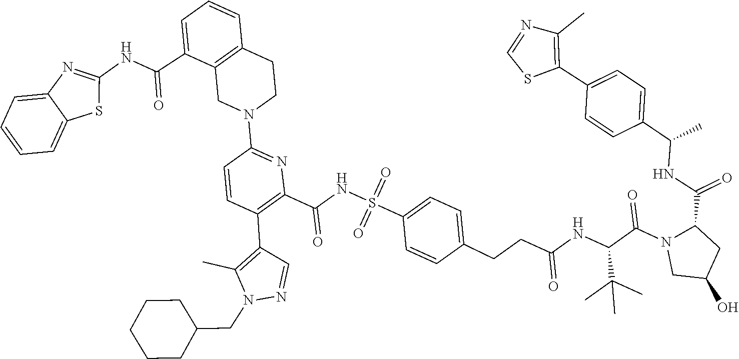 [SMILES: Cc1ncsc1-c1ccc([C@H](C)NC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-124 | 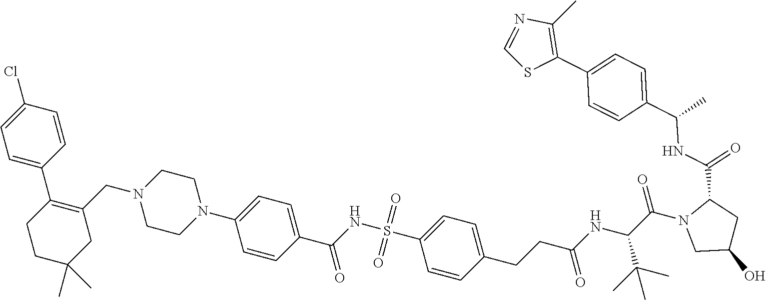 [SMILES: Cc1ncsc1-c1ccc([C@H](C)NC(=O)[C@@H]2C[C@@H](O)CN2C(=O)[C@@H](NC(=O)CCc2ccc(S(=O)(=O)NC(=O)c3ccc(N4CCN(CC5=C(c6ccc(Cl)cc6)CCC(C)(C)C5)CC4)cc3)cc2)C(C)(C)C)cc1] |
| I-125 | 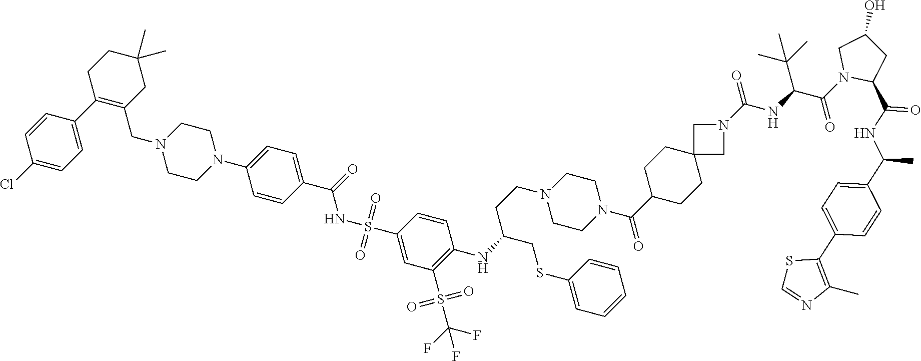 [SMILES: Cc1ncsc1-c1ccc([C@H](C)NC(=O)[C@@H]2C[C@@H](O)CN2C(=O)[C@@H](NC(=O)N2CC3(CCC(C(=O)N4CCN(CC[C@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)C2)C(C)(C)C)cc1] |
| I-126 | 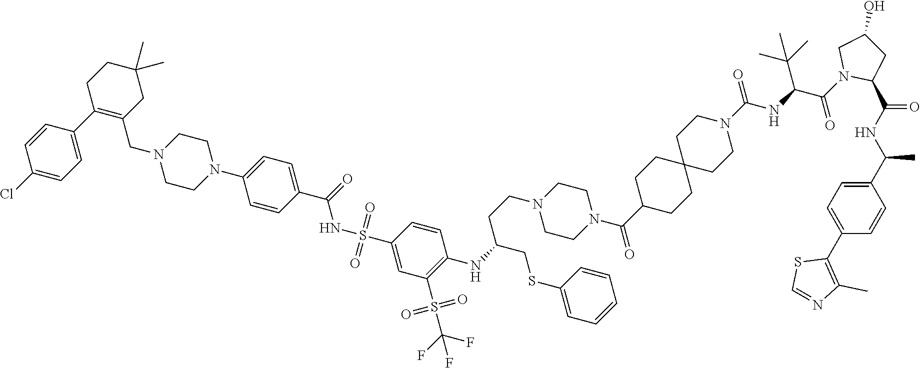 [SMILES: Cc1ncsc1-c1ccc([C@H](C)NC(=O)[C@@H]2C[C@@H](O)CN2C(=O)[C@@H](NC(=O)N2CCC3(CCC(C(=O)N4CCN(CC[C@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)CC2)C(C)(C)C)cc1] |
| I-127 | 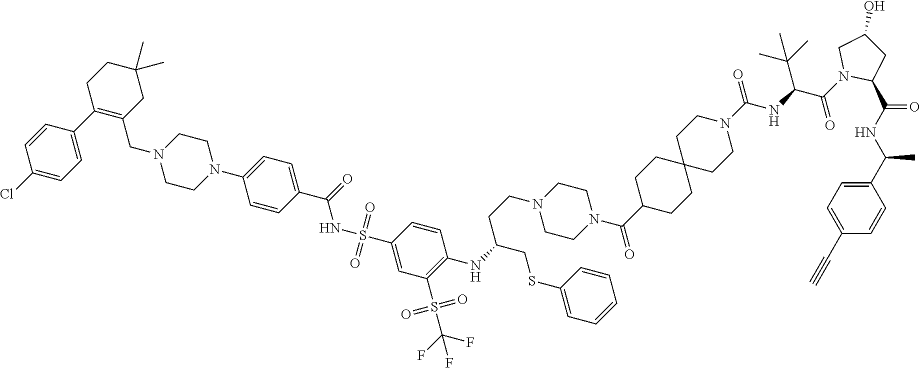 [SMILES: C#Cc1ccc([C@H](C)NC(=O)[C@@H]2C[C@@H](O)CN2C(=O)[C@@H](NC(=O)N2CCC3(CCC(C(=O)N4CCN(CC[C@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)CC2)C(C)(C)C)cc1] |
| I-128 | 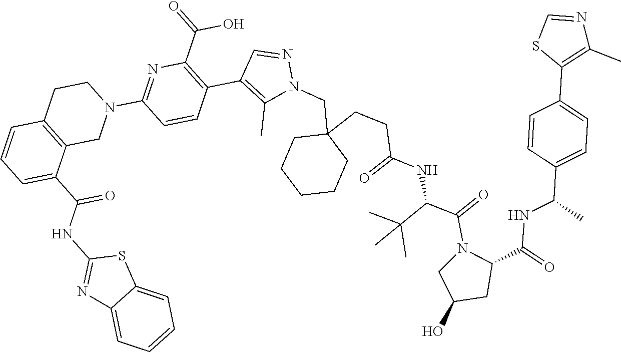 [SMILES: Cc1ncsc1-c1ccc([C@H](C)NC(=O)[C@@H]2C[C@@H](O)CN2C(=O)[C@@H](NC(=O)CCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-129 | 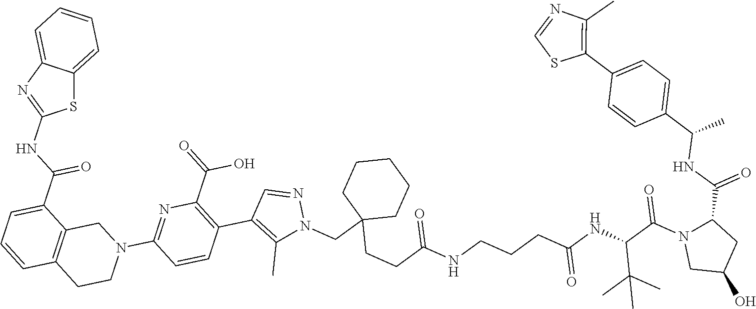 [SMILES: Cc1ncsc1-c1ccc([C@H](C)NC(=O)[C@@H]2C[C@@H](O)CN2C(=O)[C@@H](NC(=O)CCCNC(=O)CCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-130 | 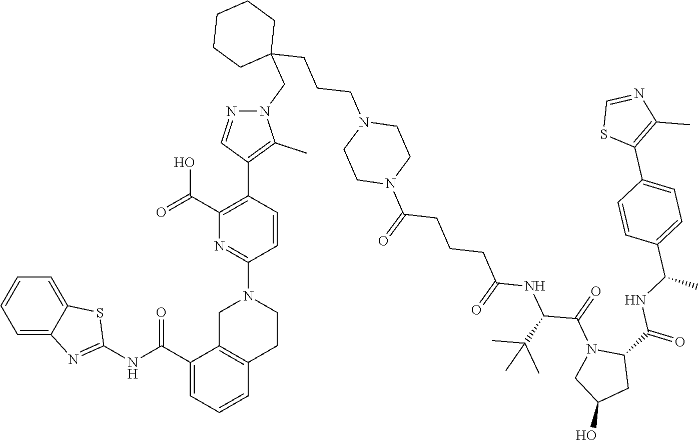 [SMILES: Cc1ncsc1-c1ccc([C@H](C)NC(=O)[C@@H]2C[C@@H](O)CN2C(=O)[C@@H](NC(=O)CCCC(=O)N2CCN(CCCC3(Cn4ncc(-c5ccc(N6CCc7cccc(C(=O)Nc8nc9ccccc9s8)c7C6)nc5C(=O)O)c4C)CCCCC3)CC2)C(C)(C)C)cc1] |
| I-131 | 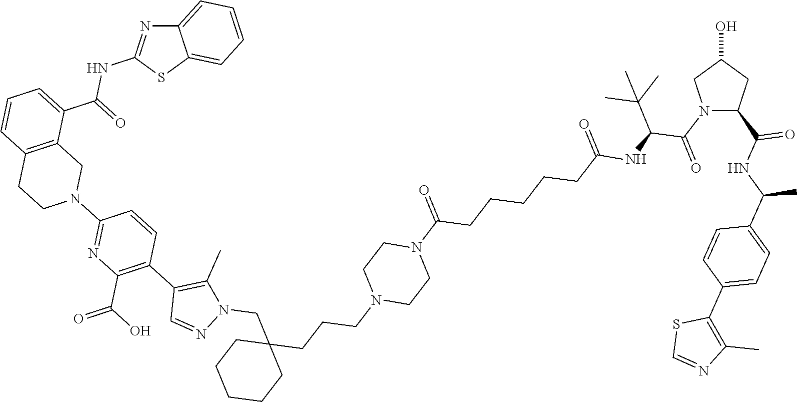 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCC3(Cn4ncc(-c5ccc(N6CCc7cccc(C(=O)Nc8nc9ccccc9s8)c7C6)nc5C(=O)O)c4C)CCCCC3)CC2)C(C)(C)C)cc1] |
| I-132 | 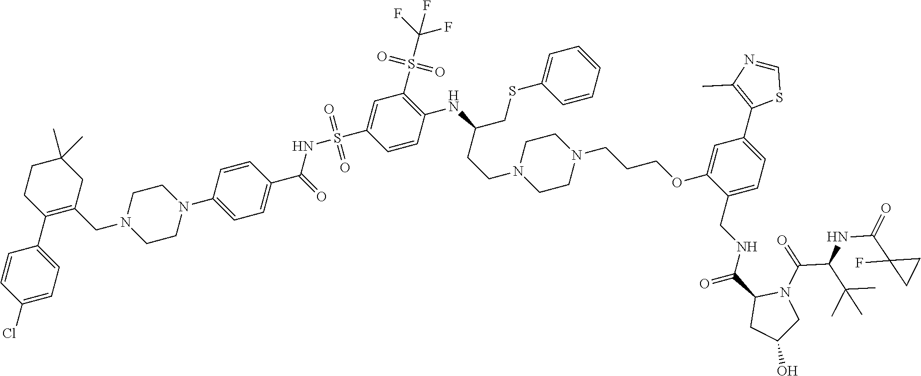 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-133 | 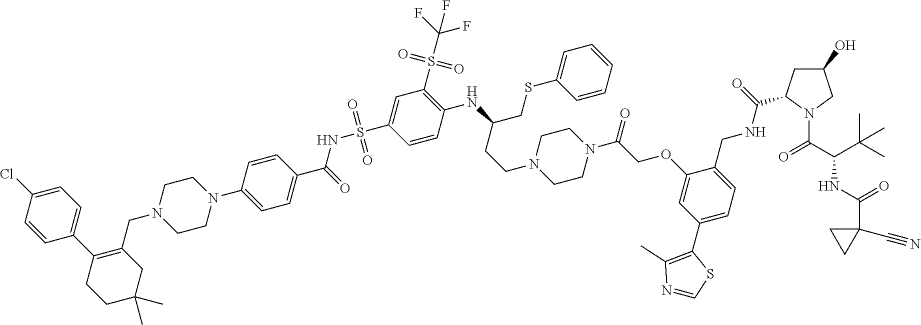 [SMILES: Cc1ncsc1-c1ccc(CNC(=O)[C@@H]2C[C@@H](O)CN2C(=O)[C@@H](NC(=O)C2(C#N)CC2)C(C)(C)C)c(OCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-134 | 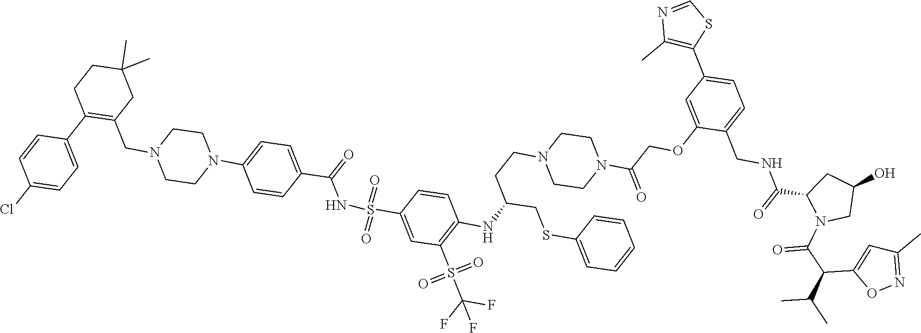 [SMILES: Cc1cc([C@H](C(=O)N2C[C@H](O)C[C@H]2C(=O)NCc2ccc(-c3scnc3C)cc2OCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-135 | 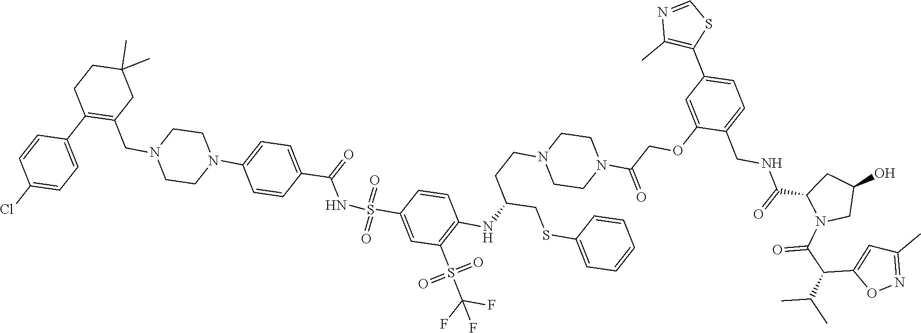 [SMILES: Cc1cc([C@@H](C(=O)N2C[C@H](O)C[C@H]2C(=O)NCc2ccc(-c3scnc3C)cc2OCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-136 | 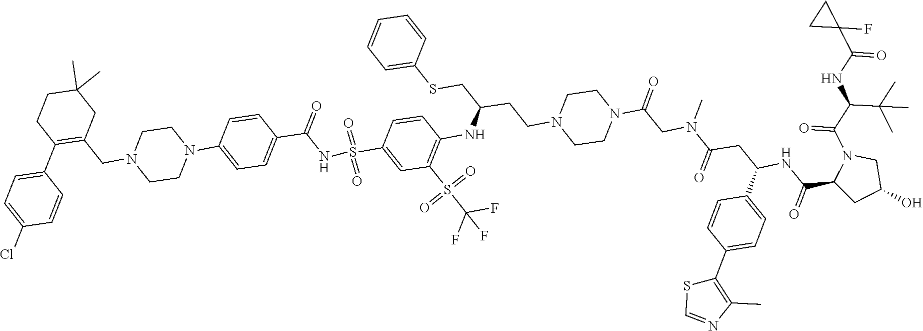 [SMILES: Cc1ncsc1-c1ccc([C@H](CC(=O)N(C)CC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-137 | 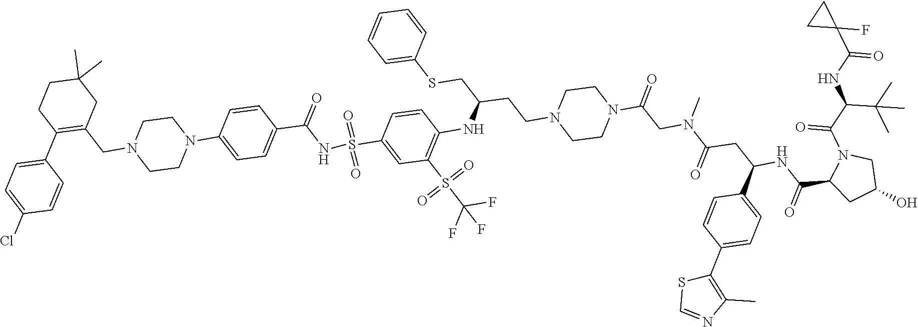 [SMILES: Cc1ncsc1-c1ccc([C@@H](CC(=O)N(C)CC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-138 | 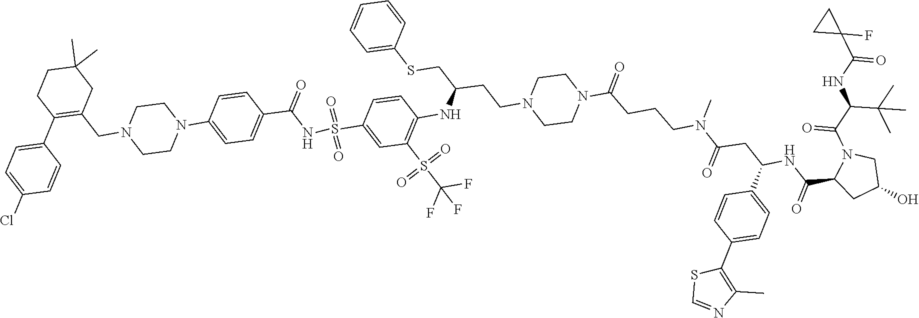 [SMILES: Cc1ncsc1-c1ccc([C@H](CC(=O)N(C)CCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-139 | 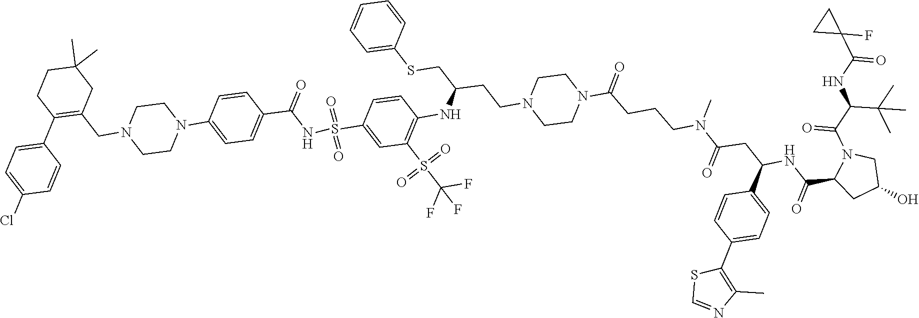 [SMILES: Cc1ncsc1-c1ccc([C@@H](CC(=O)N(C)CCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-140 | 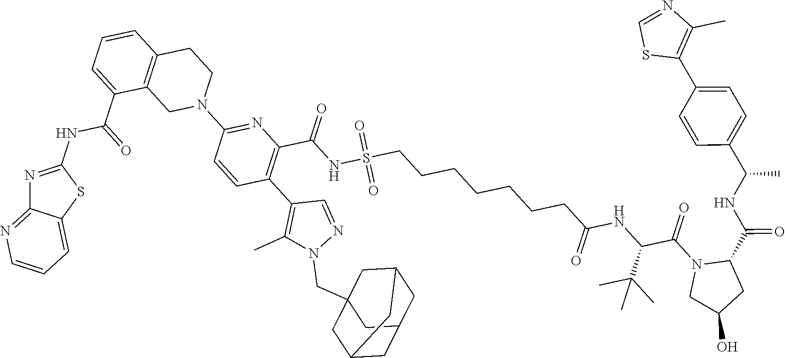 [SMILES: Cc1ncsc1-c1ccc([C@H](C)NC(=O)[C@@H]2C[C@@H](O)CN2C(=O)[C@@H](NC(=O)CCCCCCCS(=O)(=O)NC(=O)c2nc(N3CCc4cccc(C(=O)Nc5nc6ncccc6s5)c4C3)ccc2-c2cnn(CC34CC5CC(CC(C5)C3)C4)c2C)C(C)(C)C)cc1] |
| I-141 | 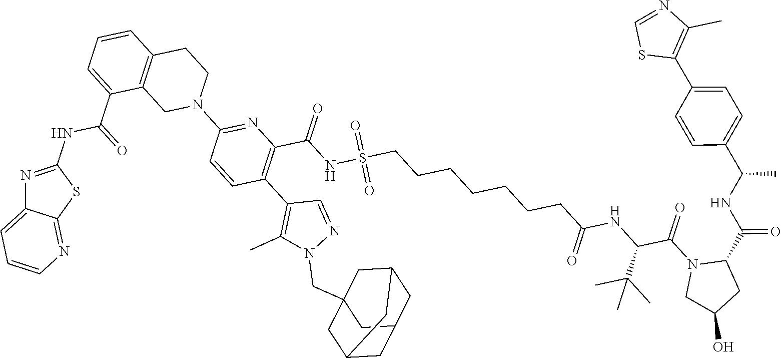 [SMILES: Cc1ncsc1-c1ccc([C@H](C)NC(=O)[C@@H]2C[C@@H](O)CN2C(=O)[C@@H](NC(=O)CCCCCCCS(=O)(=O)NC(=O)c2nc(N3CCc4cccc(C(=O)Nc5nc6cccnc6s5)c4C3)ccc2-c2cnn(CC34CC5CC(CC(C5)C3)C4)c2C)C(C)(C)C)cc1] |
| I-142 | 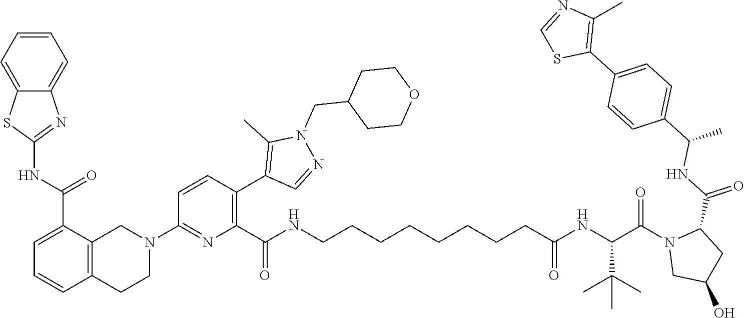 [SMILES: Cc1ncsc1-c1ccc([C@H](C)NC(=O)[C@@H]2C[C@@H](O)CN2C(=O)[C@@H](NC(=O)CCCCCCCCNC(=O)c2nc(N3CCc4cccc(C(=O)Nc5nc6ccccc6s5)c4C3)ccc2-c2cnn(CC3CCOCC3)c2C)C(C)(C)C)cc1] |
| I-143 | 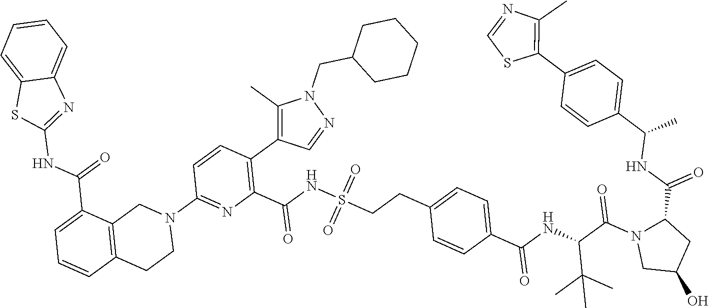 [SMILES: Cc1ncsc1-c1ccc([C@H](C)NC(=O)[C@@H]2C[C@@H](O)CN2C(=O)[C@@H](NC(=O)c2ccc(CCS(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-144 | 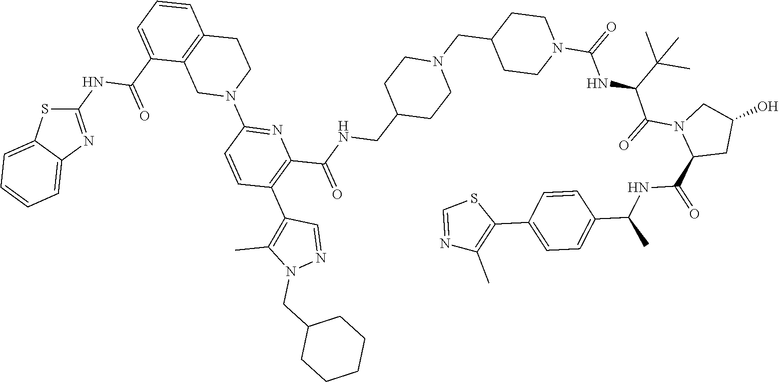 [SMILES: Cc1ncsc1-c1ccc([C@H](C)NC(=O)[C@@H]2C[C@@H](O)CN2C(=O)[C@@H](NC(=O)N2CCC(CN3CCC(CNC(=O)c4nc(N5CCc6cccc(C(=O)Nc7nc8ccccc8s7)c6C5)ccc4-c4cnn(CC5CCCCC5)c4C)CC3)CC2)C(C)(C)C)cc1] |
| I-145 | 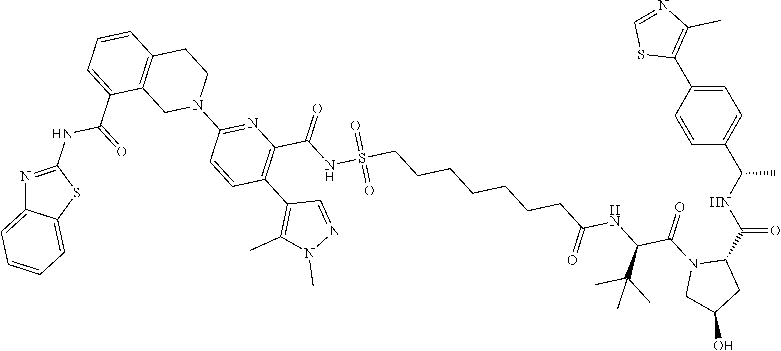 [SMILES: Cc1ncsc1-c1ccc([C@H](C)NC(=O)[C@@H]2C[C@@H](O)CN2C(=O)[C@H](NC(=O)CCCCCCCS(=O)(=O)NC(=O)c2nc(N3CCc4cccc(C(=O)Nc5nc6ccccc6s5)c4C3)ccc2-c2cnn(C)c2C)C(C)(C)C)cc1] |
| I-146 | 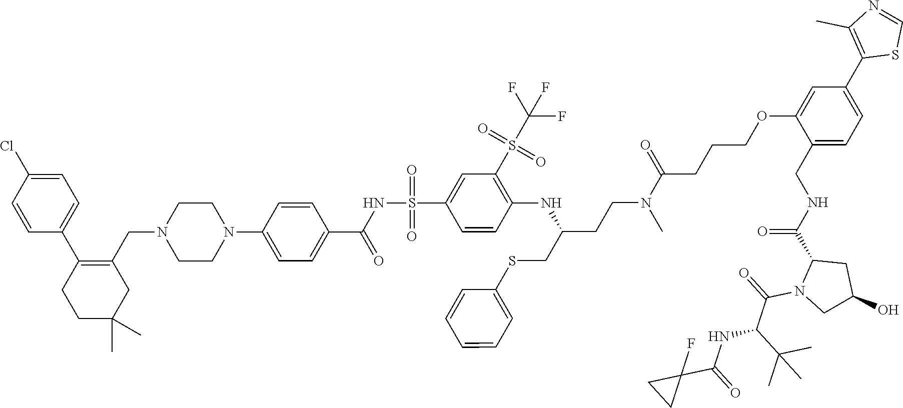 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N(C)CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)c1] |
| I-147 | 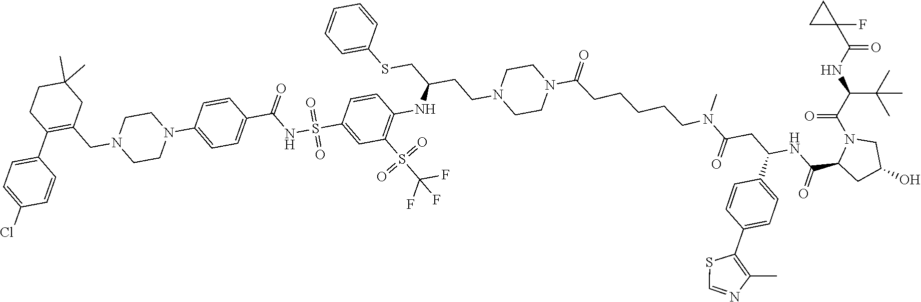 [SMILES: Cc1ncsc1-c1ccc([C@H](CC(=O)N(C)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-148 | 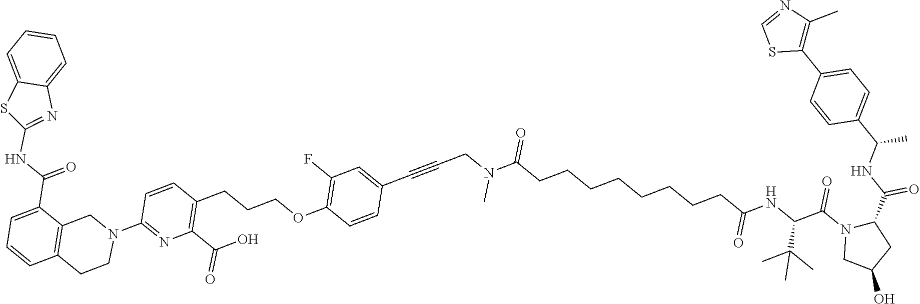 [SMILES: Cc1ncsc1-c1ccc([C@H](C)NC(=O)[C@@H]2C[C@@H](O)CN2C(=O)[C@@H](NC(=O)CCCCCCCCC(=O)N(C)CC#Cc2ccc(OCCCc3ccc(N4CCc5cccc(C(=O)Nc6nc7ccccc7s6)c5C4)nc3C(=O)O)c(F)c2)C(C)(C)C)cc1] |
| I-149 | 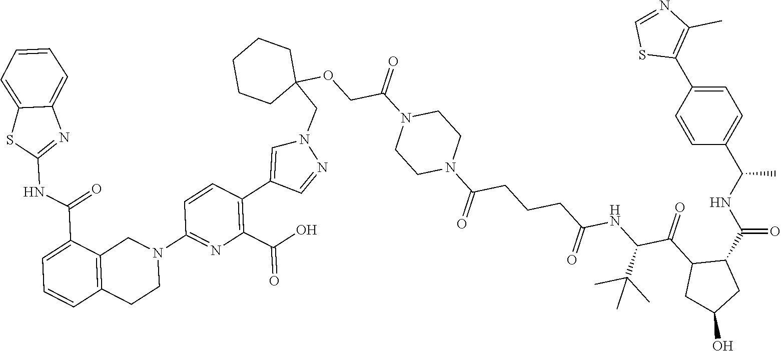 [SMILES: Cc1ncsc1-c1ccc([C@H](C)NC(=O)[C@@H]2C[C@@H](O)CC2C(=O)[C@@H](NC(=O)CCCC(=O)N2CCN(C(=O)COC3(Cn4cc(-c5ccc(N6CCc7cccc(C(=O)Nc8nc9ccccc9s8)c7C6)nc5C(=O)O)cn4)CCCCC3)CC2)C(C)(C)C)cc1] |
| I-150 | 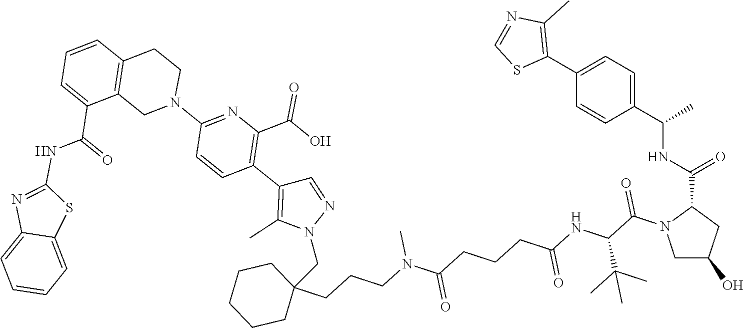 [SMILES: Cc1ncsc1-c1ccc([C@H](C)NC(=O)[C@@H]2C[C@@H](O)CN2C(=O)[C@@H](NC(=O)CCCC(=O)N(C)CCCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-151 | 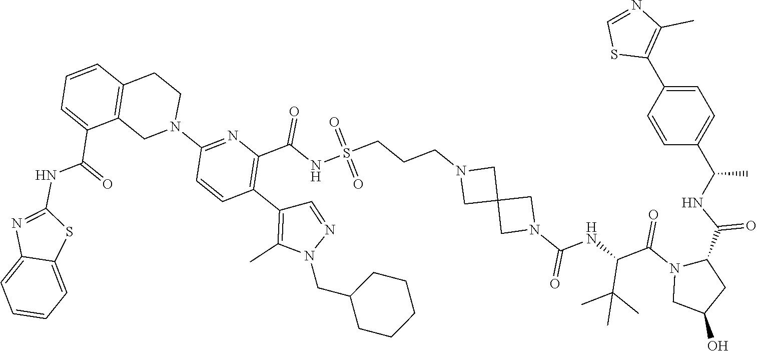 [SMILES: Cc1ncsc1-c1ccc([C@H](C)NC(=O)[C@@H]2C[C@@H](O)CN2C(=O)[C@@H](NC(=O)N2CC3(CN(CCCS(=O)(=O)NC(=O)c4nc(N5CCc6cccc(C(=O)Nc7nc8ccccc8s7)c6C5)ccc4-c4cnn(CC5CCCCC5)c4C)C3)C2)C(C)(C)C)cc1] |
| I-152 | 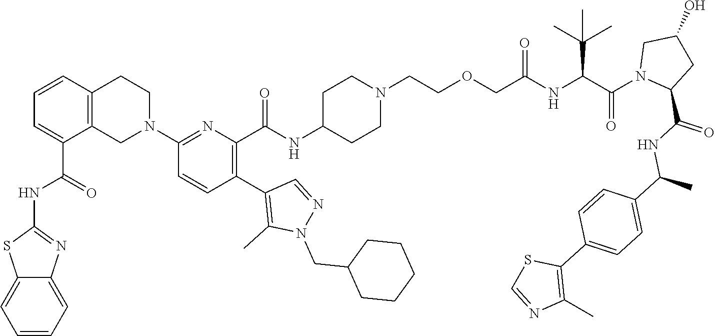 [SMILES: Cc1ncsc1-c1ccc([C@H](C)NC(=O)[C@@H]2C[C@@H](O)CN2C(=O)[C@@H](NC(=O)COCCN2CCC(NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-153 | 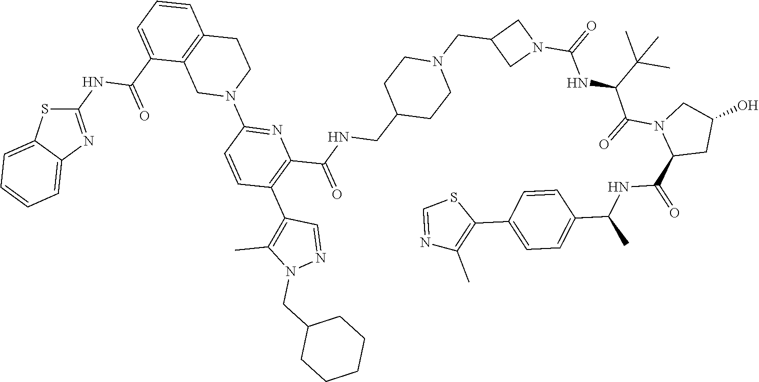 [SMILES: Cc1ncsc1-c1ccc([C@H](C)NC(=O)[C@@H]2C[C@@H](O)CN2C(=O)[C@@H](NC(=O)N2CC(CN3CCC(CNC(=O)c4nc(N5CCc6cccc(C(=O)Nc7nc8ccccc8s7)c6C5)ccc4-c4cnn(CC5CCCCC5)c4C)CC3)C2)C(C)(C)C)cc1] |
| I-154 | 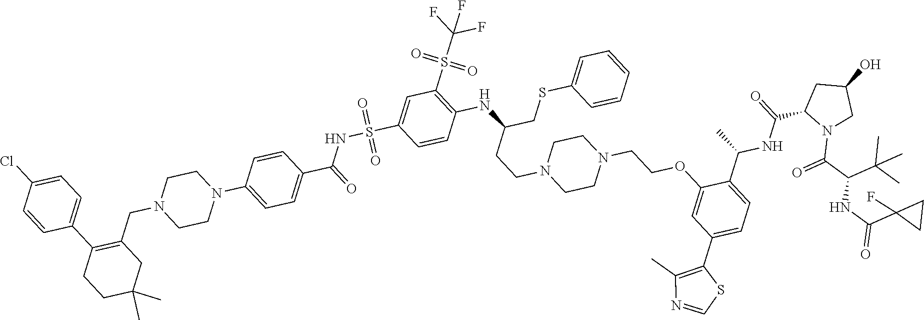 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(OCCN2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-155 | 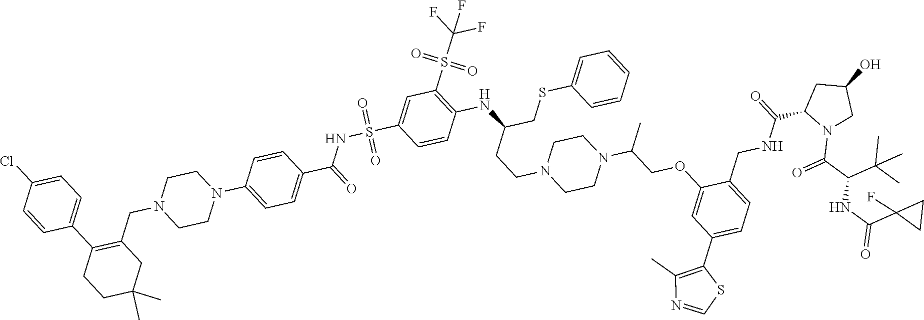 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(C)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-156 | 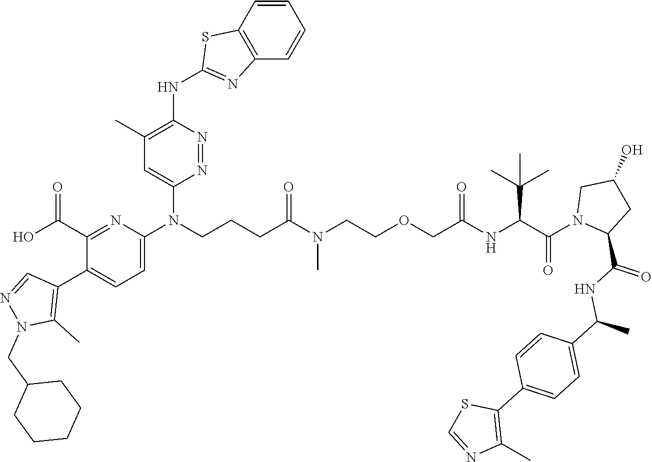 [SMILES: Cc1cc(N(CCCC(=O)N(C)CCOCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC4CCCCC4)c3C)c(C(=O)O)n2)nnc1Nc1nc2ccccc2s1] |
| I-157 | 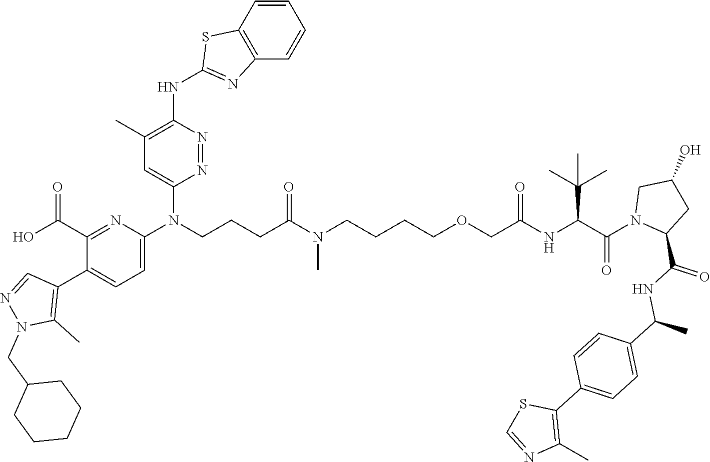 [SMILES: Cc1cc(N(CCCC(=O)N(C)CCCCOCC(=O)N[C@H](C(=O)N2C[C@H](O)C[C@H]2C(=O)N[C@@H](C)c2ccc(-c3scnc3C)cc2)C(C)(C)C)c2ccc(-c3cnn(CC4CCCCC4)c3C)c(C(=O)O)n2)nnc1Nc1nc2ccccc2s1] |
| I-158 | 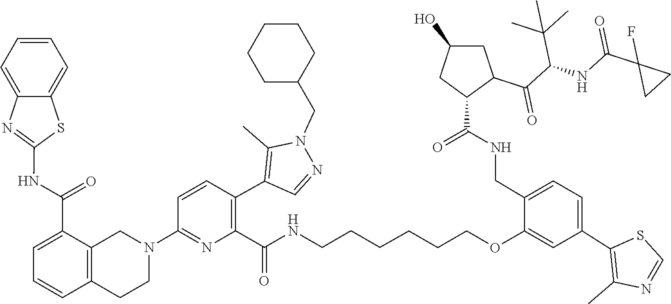 [SMILES: Cc1ncsc1-c1ccc(CNC(=O)[C@@H]2C[C@@H](O)CC2C(=O)[C@@H](NC(=O)C2(F)CC2)C(C)(C)C)c(OCCCCCCNC(=O)c2nc(N3CCc4cccc(C(=O)Nc5nc6ccccc6s5)c4C3)ccc2-c2cnn(CC3CCCCC3)c2C)c1] |
| I-159 | 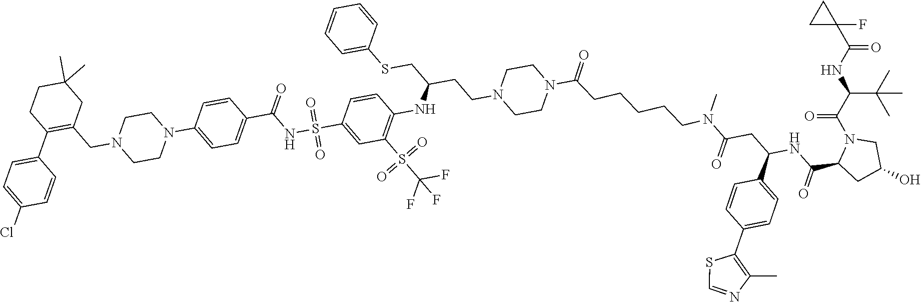 [SMILES: Cc1ncsc1-c1ccc([C@@H](CC(=O)N(C)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-160 | 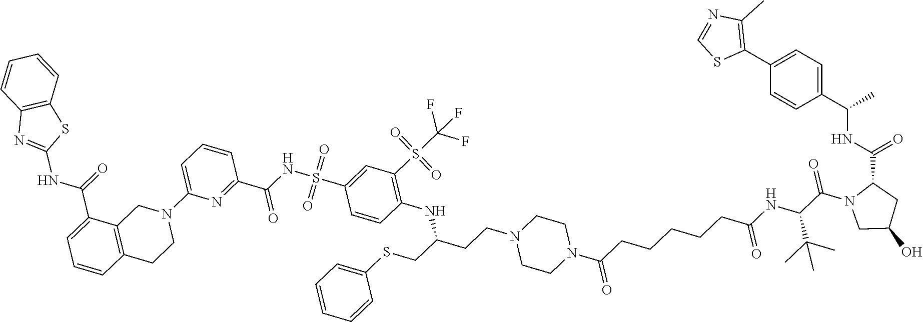 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4cccc(N5CCc6cccc(C(=O)Nc7nc8ccccc8s7)c6C5)n4)cc3S(=O)(=O)C(F)(F)F)CC2)C(C)(C)C)cc1] |
| I-161 | 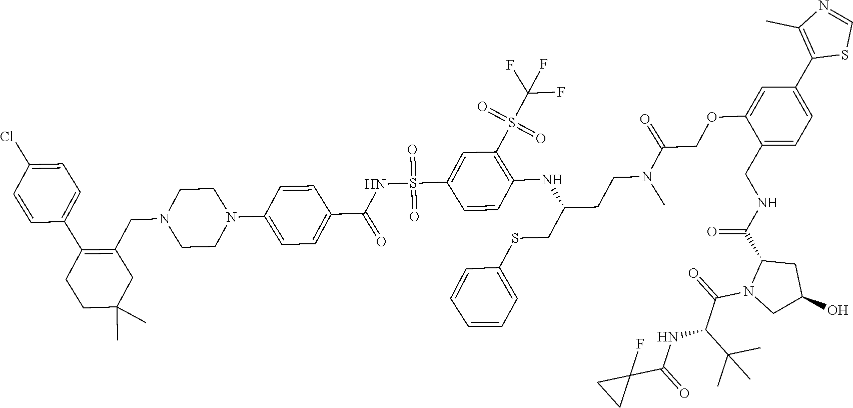 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N(C)CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)c1] |
| I-162 | 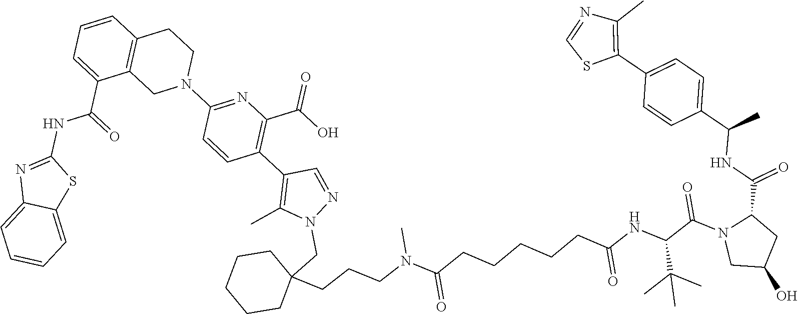 [SMILES: Cc1ncsc1-c1ccc([C@@H](C)NC(=O)[C@@H]2C[C@@H](O)CN2C(=O)[C@@H](NC(=O)CCCCCC(=O)N(C)CCCC2(Cn3ncc(-c4ccc(N5CCc6cccc(C(=O)Nc7nc8ccccc8s7)c6C5)nc4C(=O)O)c3C)CCCCC2)C(C)(C)C)cc1] |
| I-163 | 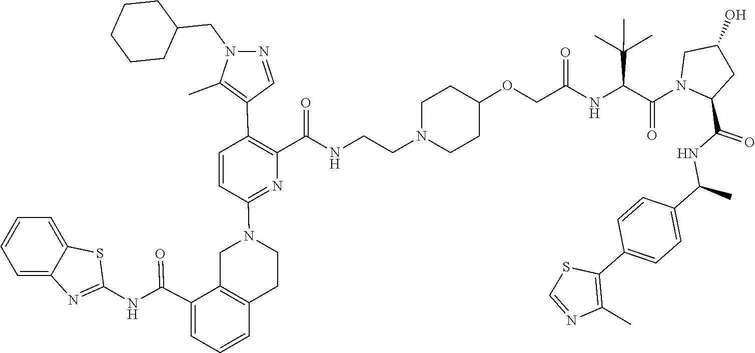 [SMILES: Cc1ncsc1-c1ccc([C@H](C)NC(=O)[C@@H]2C[C@@H](O)CN2C(=O)[C@@H](NC(=O)COC2CCN(CCNC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-164 | 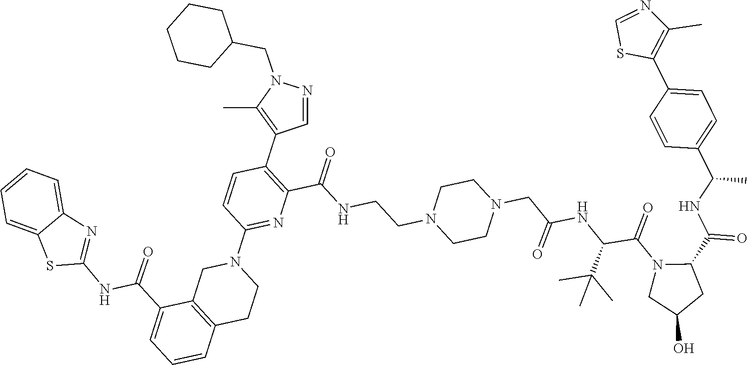 [SMILES: Cc1ncsc1-c1ccc([C@H](C)NC(=O)[C@@H]2C[C@@H](O)CN2C(=O)[C@@H](NC(=O)CN2CCN(CCNC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-165 | 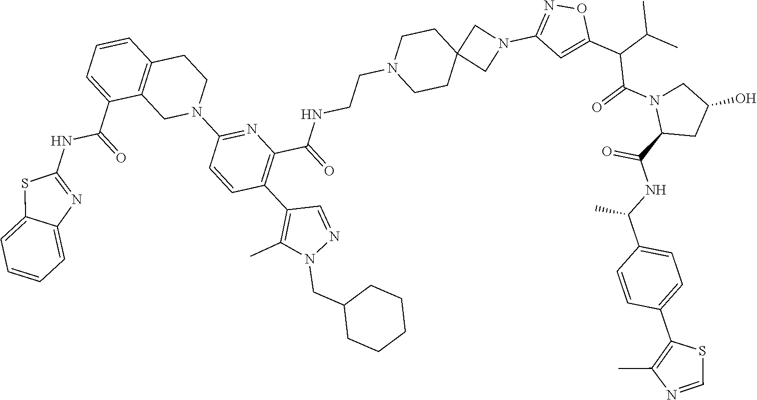 [SMILES: Cc1ncsc1-c1ccc([C@H](C)NC(=O)[C@@H]2C[C@@H](O)CN2C(=O)C(c2cc(N3CC4(CCN(CCNC(=O)c5nc(N6CCc7cccc(C(=O)Nc8nc9ccccc9s8)c7C6)ccc5-c5cnn(CC6CCCCC6)c5C)CC4)C3)no2)C(C)C)cc1] |
| I-166 | 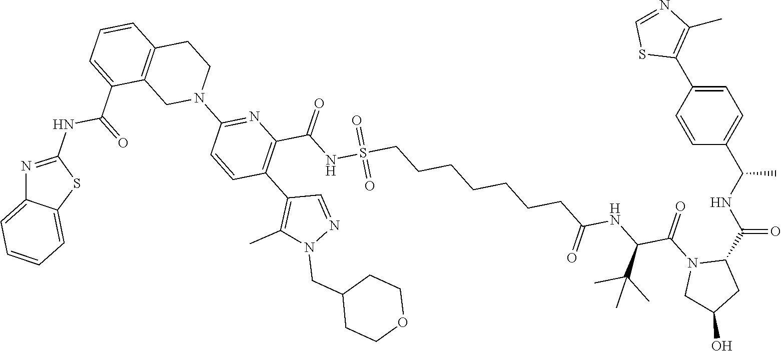 [SMILES: Cc1ncsc1-c1ccc([C@H](C)NC(=O)[C@@H]2C[C@@H](O)CN2C(=O)[C@H](NC(=O)CCCCCCCS(=O)(=O)NC(=O)c2nc(N3CCc4cccc(C(=O)Nc5nc6ccccc6s5)c4C3)ccc2-c2cnn(CC3CCOCC3)c2C)C(C)(C)C)cc1] |
| I-167 | 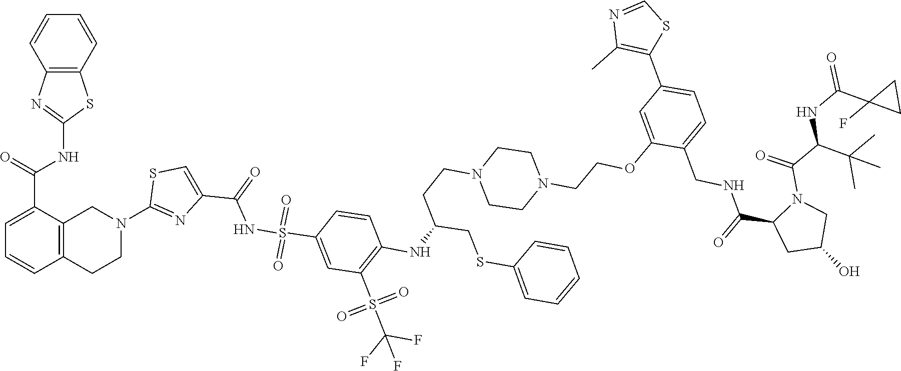 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@H](CSc3ccccc3)Nc3ccc(S(=O)(=O)NC(=O)c4csc(N5CCc6cccc(C(=O)Nc7nc8ccccc8s7)c6C5)n4)cc3S(=O)(=O)C(F)(F)F)CC2)c1] |
| I-168 | 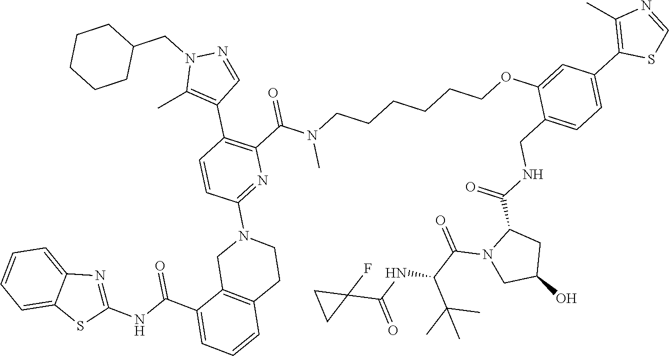 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCN(C)C(=O)c2nc(N3CCc4cccc(C(=O)Nc5nc6ccccc6s5)c4C3)ccc2-c2cnn(CC3CCCCC3)c2C)c1] |
| I-169 | 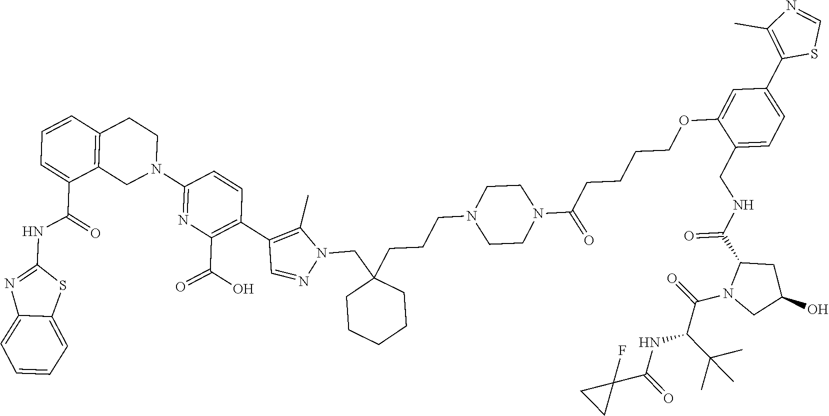 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC(=O)N2CCN(CCCC3(Cn4ncc(-c5ccc(N6CCc7cccc(C(=O)Nc8nc9ccccc9s8)c7C6)nc5C(=O)O)c4C)CCCCC3)CC2)c1] |
| I-170 | 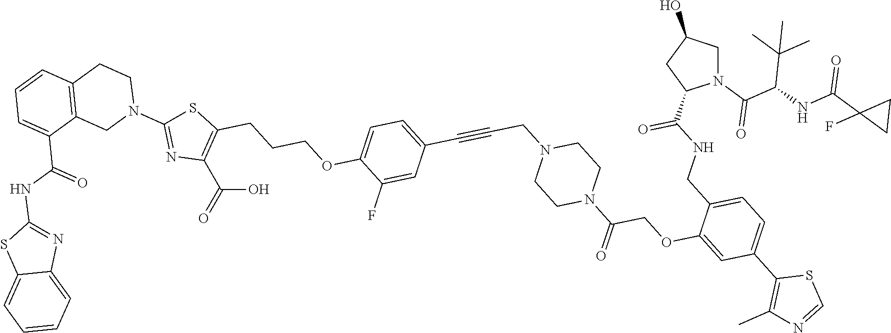 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N2CCN(CC#Cc3ccc(OCCCc4sc(N5CCc6cccc(C(=O)Nc7nc8ccccc8s7)c6C5)nc4C(=O)O)c(F)c3)CC2)c1] |
| I-171 | 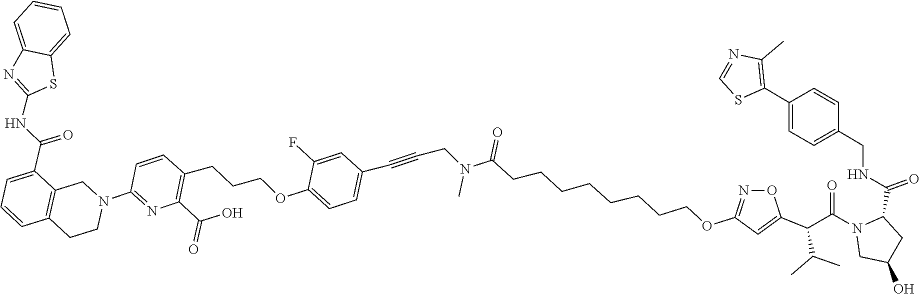 [SMILES: Cc1ncsc1-c1ccc(CNC(=O)[C@@H]2C[C@@H](O)CN2C(=O)[C@@H](c2cc(OCCCCCCCCC(=O)N(C)CC#Cc3ccc(OCCCc4ccc(N5CCc6cccc(C(=O)Nc7nc8ccccc8s7)c6C5)nc4C(=O)O)c(F)c3)no2)C(C)C)cc1] |
| I-172 | 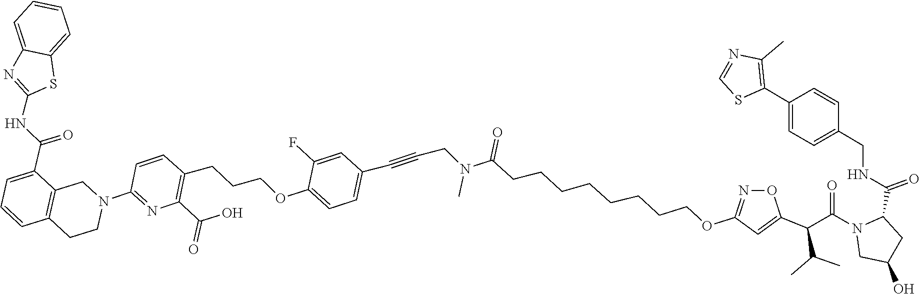 [SMILES: Cc1ncsc1-c1ccc(CNC(=O)[C@@H]2C[C@@H](O)CN2C(=O)[C@H](c2cc(OCCCCCCCCC(=O)N(C)CC#Cc3ccc(OCCCc4ccc(N5CCc6cccc(C(=O)Nc7nc8ccccc8s7)c6C5)nc4C(=O)O)c(F)c3)no2)C(C)C)cc1] |
| I-173 | 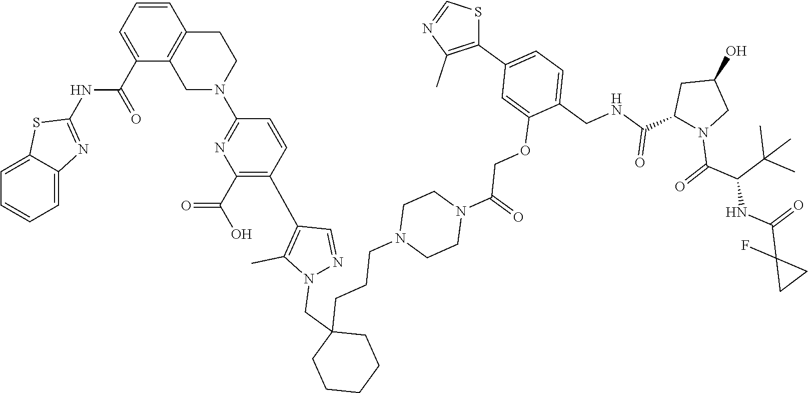 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N2CCN(CCCC3(Cn4ncc(-c5ccc(N6CCc7cccc(C(=O)Nc8nc9ccccc9s8)c7C6)nc5C(=O)O)c4C)CCCCC3)CC2)c1] |
| I-174 | 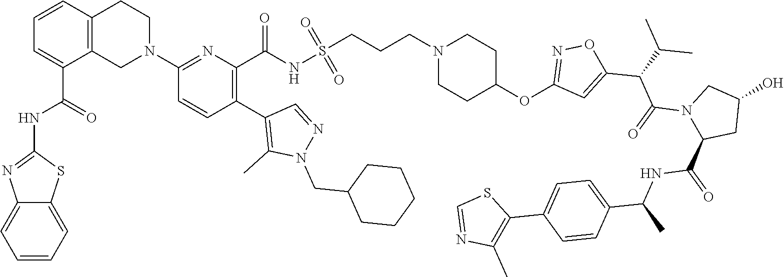 [SMILES: Cc1ncsc1-c1ccc([C@H](C)NC(=O)[C@@H]2C[C@@H](O)CN2C(=O)[C@H](c2cc(OC3CCN(CCCS(=O)(=O)NC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-175 | 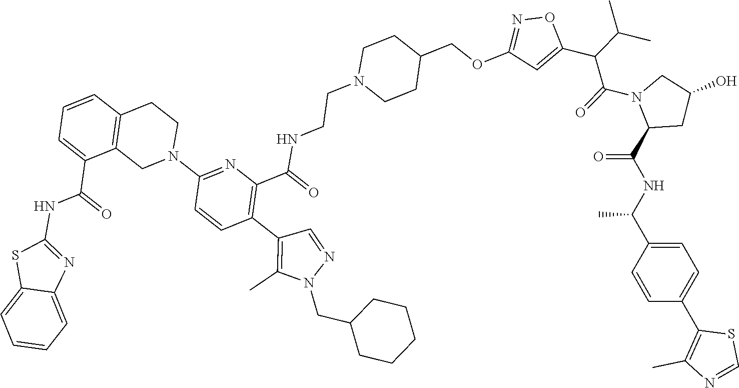 [SMILES: Cc1ncsc1-c1ccc([C@H](C)NC(=O)[C@@H]2C[C@@H](O)CN2C(=O)C(c2cc(OCC3CCN(CCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-176 | 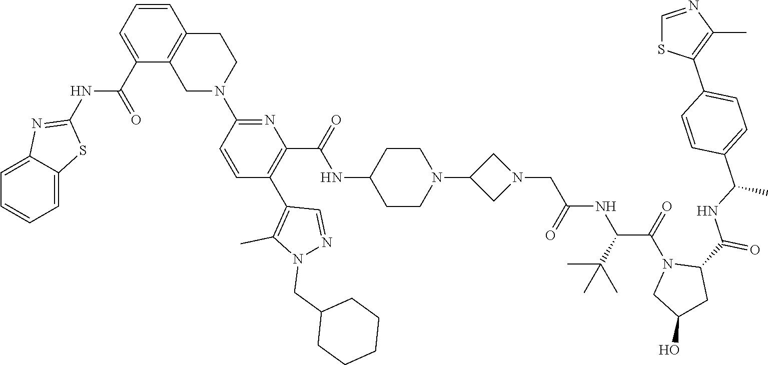 [SMILES: Cc1ncsc1-c1ccc([C@H](C)NC(=O)[C@@H]2C[C@@H](O)CN2C(=O)[C@@H](NC(=O)CN2CC(N3CCC(NC(=O)c4nc(N5CCc6cccc(C(=O)Nc7nc8ccccc8s7)c6C5)ccc4-c4cnn(CC5CCCCC5)c4C)CC3)C2)C(C)(C)C)cc1] |
| I-177 | 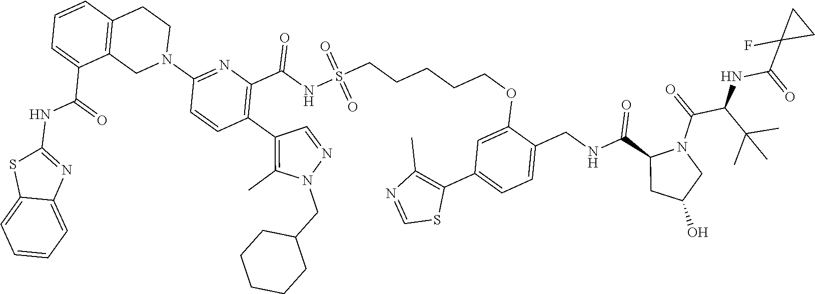 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCS(=O)(=O)NC(=O)c2nc(N3CCc4cccc(C(=O)Nc5nc6ccccc6s5)c4C3)ccc2-c2cnn(CC3CCCCC3)c2C)c1] |
| I-178 | 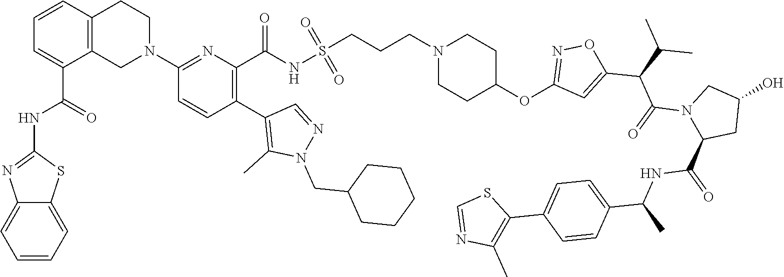 [SMILES: Cc1ncsc1-c1ccc([C@H](C)NC(=O)[C@@H]2C[C@@H](O)CN2C(=O)[C@@H](c2cc(OC3CCN(CCCS(=O)(=O)NC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-179 | 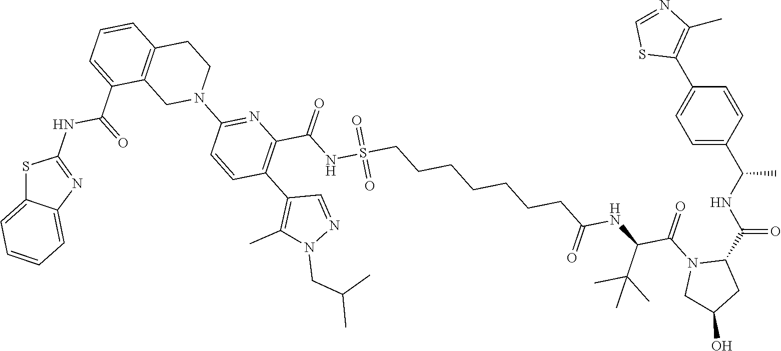 [SMILES: Cc1ncsc1-c1ccc([C@H](C)NC(=O)[C@@H]2C[C@@H](O)CN2C(=O)[C@H](NC(=O)CCCCCCCS(=O)(=O)NC(=O)c2nc(N3CCc4cccc(C(=O)Nc5nc6ccccc6s5)c4C3)ccc2-c2cnn(CC(C)C)c2C)C(C)(C)C)cc1] |
| I-180 | 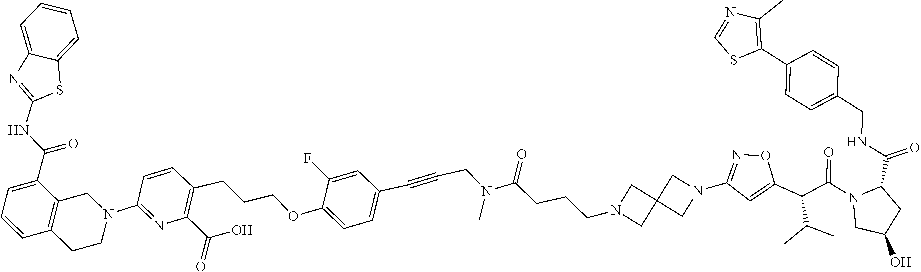 [SMILES: Cc1ncsc1-c1ccc(CNC(=O)[C@@H]2C[C@@H](O)CN2C(=O)[C@@H](c2cc(N3CC4(CN(CCCC(=O)N(C)CC#Cc5ccc(OCCCc6ccc(N7CCc8cccc(C(=O)Nc9nc%10ccccc%10s9)c8C7)nc6C(=O)O)c(F)c5)C4)C3)no2)C(C)C)cc1] |
| I-181 | 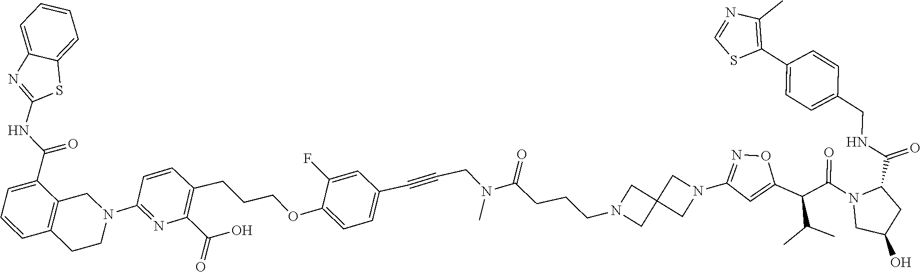 [SMILES: Cc1ncsc1-c1ccc(CNC(=O)[C@@H]2C[C@@H](O)CN2C(=O)[C@H](c2cc(N3CC4(CN(CCCC(=O)N(C)CC#Cc5ccc(OCCCc6ccc(N7CCc8cccc(C(=O)Nc9nc%10ccccc%10s9)c8C7)nc6C(=O)O)c(F)c5)C4)C3)no2)C(C)C)cc1] |
| I-182 | 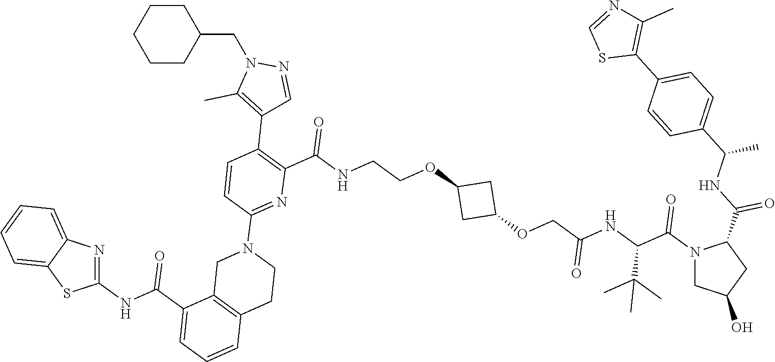 [SMILES: Cc1ncsc1-c1ccc([C@H](C)NC(=O)[C@@H]2C[C@@H](O)CN2C(=O)[C@@H](NC(=O)CO[C@H]2C[C@H](OCCNC(=O)c3nc(N4CCc5cccc(C(=O)Nc6nc7ccccc7s6)c5C4)ccc3-c3cnn(CC4CCCCC4)c3C)C2)C(C)(C)C)cc1] |
| I-183 | 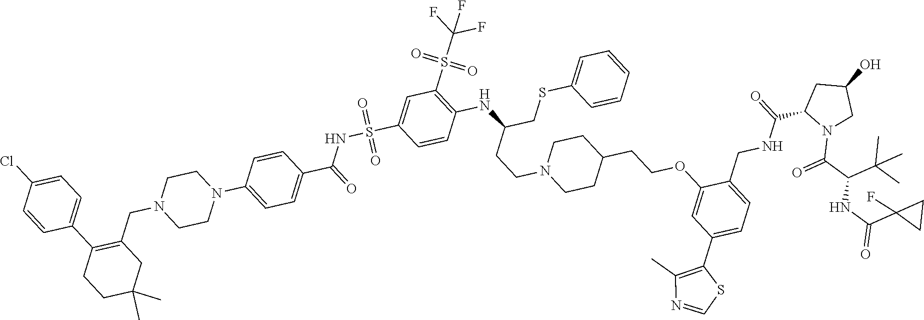 [SMILES: Cc1ncsc1-c1ccc(CNC(=O)[C@@H]2C[C@@H](O)CN2C(=O)[C@@H](NC(=O)C2(F)CC2)C(C)(C)C)c(OCCC2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-184 | 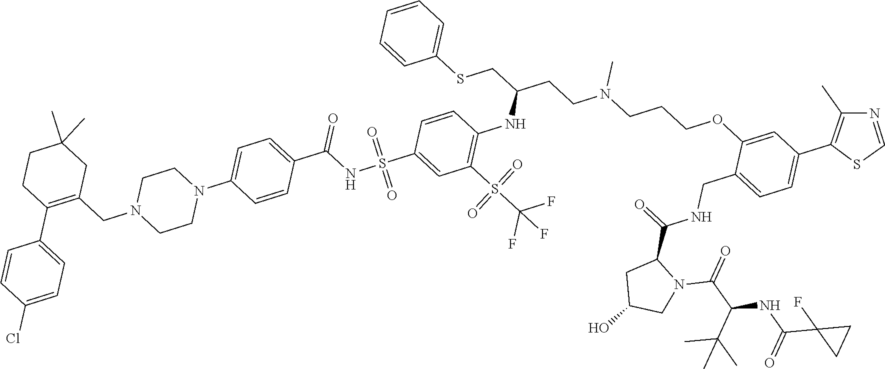 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN(C)CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)c1] |
| I-185 | 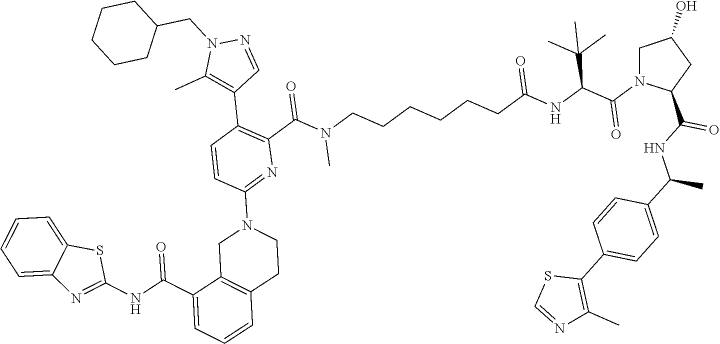 [SMILES: Cc1ncsc1-c1ccc([C@H](C)NC(=O)[C@@H]2C[C@@H](O)CN2C(=O)[C@@H](NC(=O)CCCCCCN(C)C(=O)c2nc(N3CCc4cccc(C(=O)Nc5nc6ccccc6s5)c4C3)ccc2-c2cnn(CC3CCCCC3)c2C)C(C)(C)C)cc1] |
| I-186 | 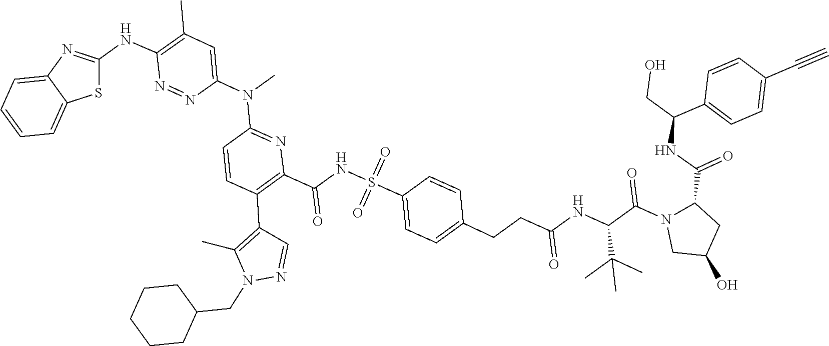 [SMILES: C#Cc1ccc([C@H](CO)NC(=O)[C@@H]2C[C@@H](O)CN2C(=O)[C@@H](NC(=O)CCc2ccc(S(=O)(=O)NC(=O)c3nc(N(C)c4cc(C)c(Nc5nc6ccccc6s5)nn4)ccc3-c3cnn(CC4CCCCC4)c3C)cc2)C(C)(C)C)cc1] |
| I-187 | 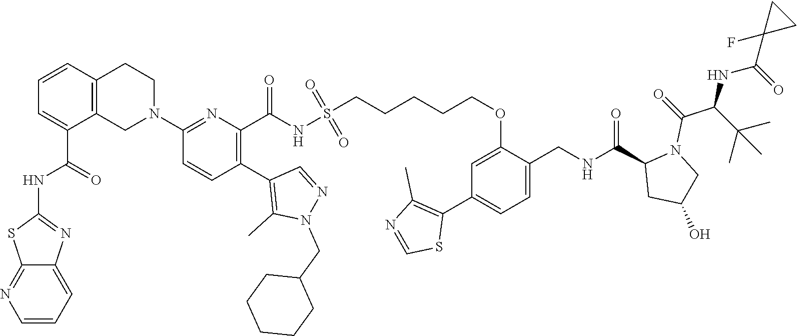 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCS(=O)(=O)NC(=O)c2nc(N3CCc4cccc(C(=O)Nc5nc6cccnc6s5)c4C3)ccc2-c2cnn(CC3CCCCC3)c2C)c1] |
| I-188 | 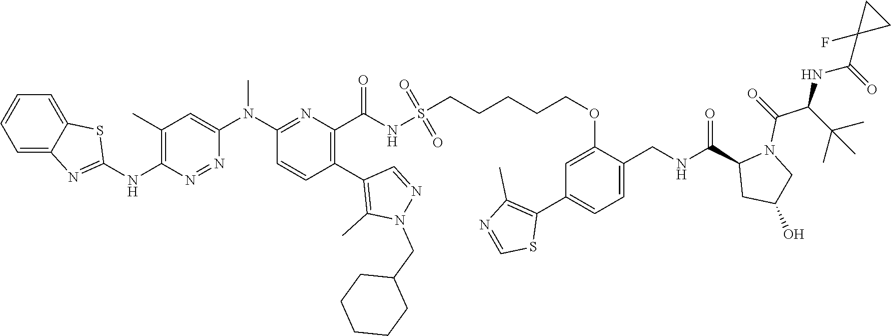 [SMILES: Cc1cc(N(C)c2ccc(-c3cnn(CC4CCCCC4)c3C)c(C(=O)NS(=O)(=O)CCCCCOc3cc(-c4scnc4C)ccc3CNC(=O)[C@@H]3C[C@@H](O)CN3C(=O)[C@@H](NC(=O)C3(F)CC3)C(C)(C)C)n2)nnc1Nc1nc2ccccc2s1] |
| I-189 | 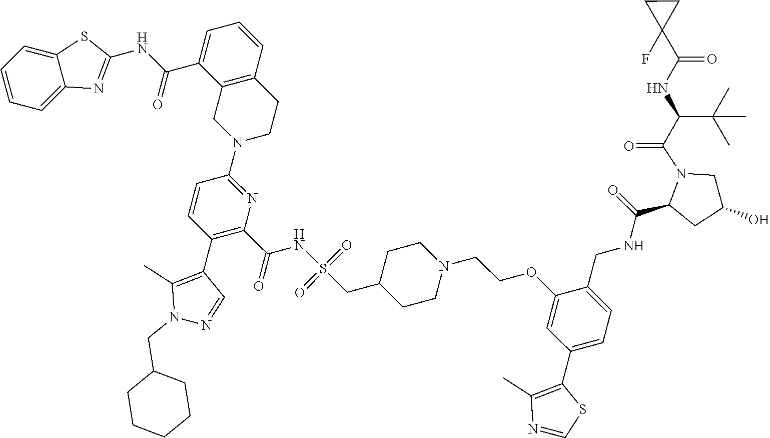 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC(CS(=O)(=O)NC(=O)c3nc(N4CCc5cccc(C(=O)Nc6nc7ccccc7s6)c5C4)ccc3-c3cnn(CC4CCCCC4)c3C)CC2)c1] |
| I-190 | 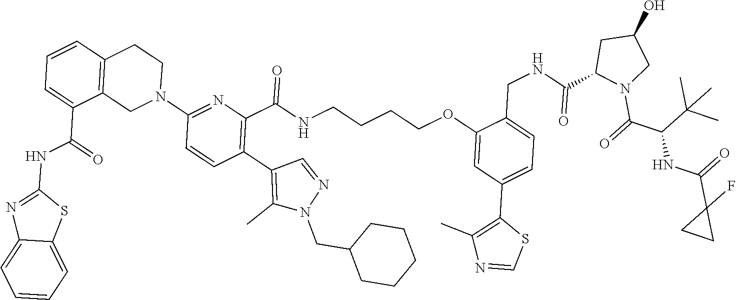 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCNC(=O)c2nc(N3CCc4cccc(C(=O)Nc5nc6ccccc6s5)c4C3)ccc2-c2cnn(CC3CCCCC3)c2C)c1] |
| I-191 | 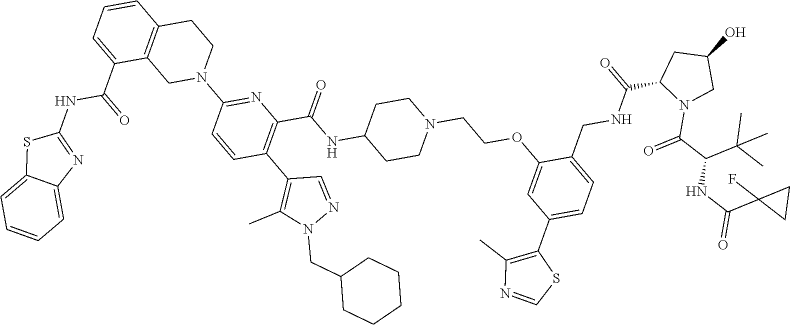 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC(NC(=O)c3nc(N4CCc5cccc(C(=O)Nc6nc7ccccc7s6)c5C4)ccc3-c3cnn(CC4CCCCC4)c3C)CC2)c1] |
| I-192 | 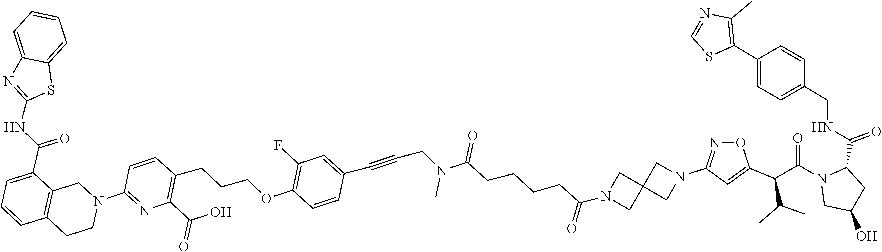 [SMILES: Cc1ncsc1-c1ccc(CNC(=O)[C@@H]2C[C@@H](O)CN2C(=O)[C@H](c2cc(N3CC4(CN(C(=O)CCCCC(=O)N(C)CC#Cc5ccc(OCCCc6ccc(N7CCc8cccc(C(=O)Nc9nc%10ccccc%10s9)c8C7)nc6C(=O)O)c(F)c5)C4)C3)no2)C(C)C)cc1] |
| I-193 | 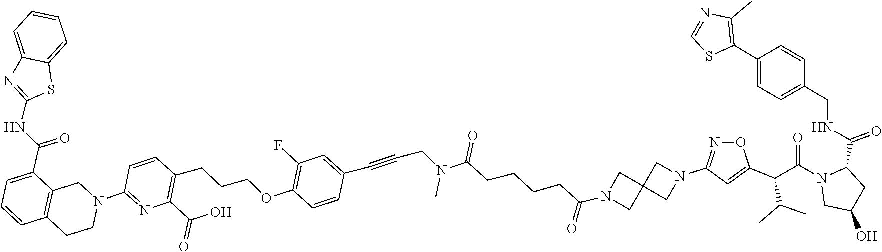 [SMILES: Cc1ncsc1-c1ccc(CNC(=O)[C@@H]2C[C@@H](O)CN2C(=O)[C@@H](c2cc(N3CC4(CN(C(=O)CCCCC(=O)N(C)CC#Cc5ccc(OCCCc6ccc(N7CCc8cccc(C(=O)Nc9nc%10ccccc%10s9)c8C7)nc6C(=O)O)c(F)c5)C4)C3)no2)C(C)C)cc1] |
| I-194 | 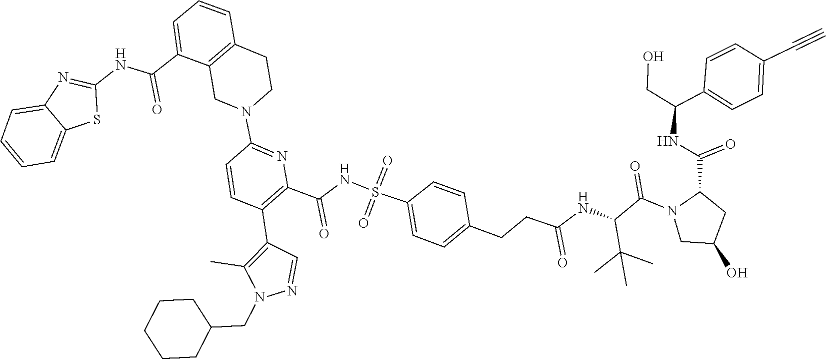 [SMILES: C#Cc1ccc([C@H](CO)NC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-195 | 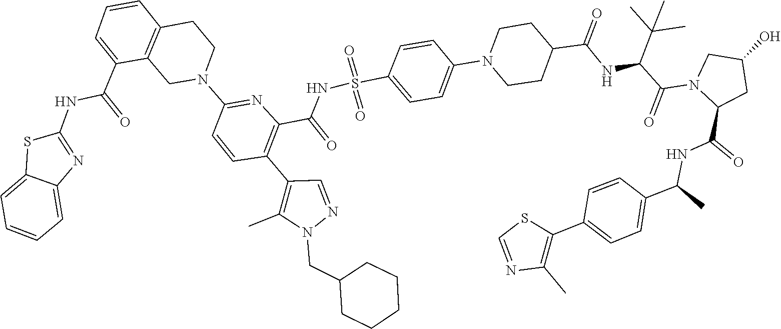 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-196 | 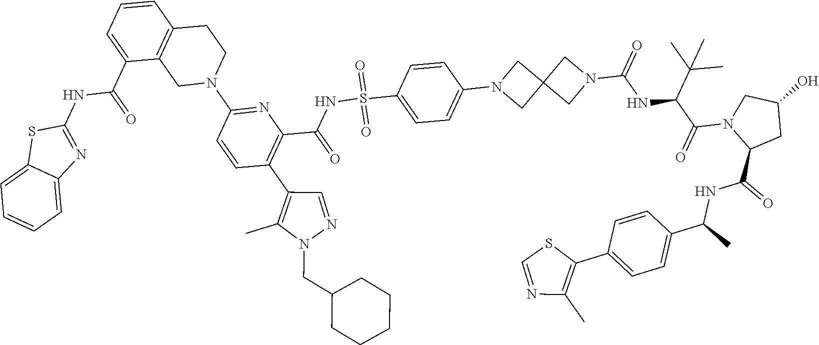 [SMILES: Cc1ncsc1-c1ccc([C@H](C)NC(=O)[C@@H]2C[C@@H](O)CN2C(=O)[C@@H](NC(=O)N2CC3(C2)CN(c2ccc(S(=O)(=O)NC(=O)c4nc(N5CCc6cccc(C(=O)Nc7nc8ccccc8s7)c6C5)ccc4-c4cnn(CC5CCCCC5)c4C)cc2)C3)C(C)(C)C)cc1] |
| I-197 | 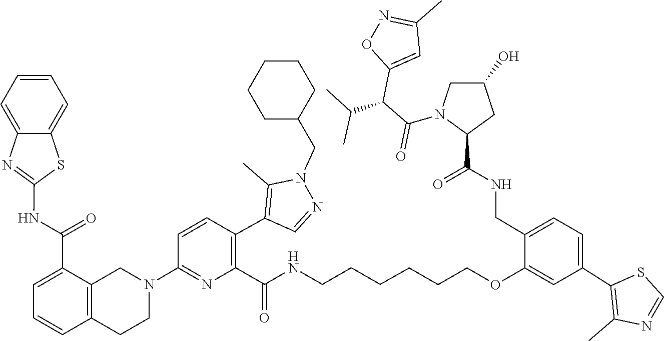 [SMILES: Cc1cc([C@H](C(=O)N2C[C@H](O)C[C@H]2C(=O)NCc2ccc(-c3scnc3C)cc2OCCCCCCNC(=O)c2nc(N3CCc4cccc(C(=O)Nc5nc6ccccc6s5)c4C3)ccc2-c2cnn(CC3CCCCC3)c2C)C(C)C)on1] |
| I-198 | 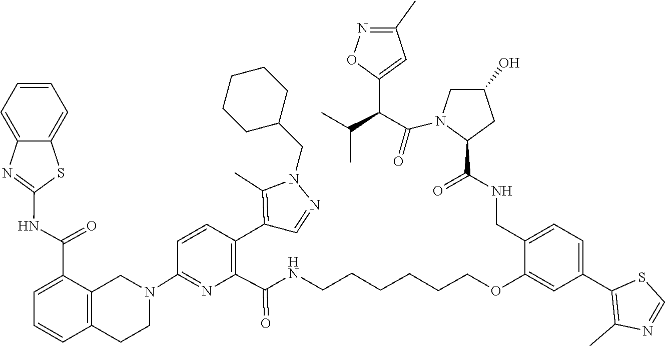 [SMILES: Cc1cc([C@@H](C(=O)N2C[C@H](O)C[C@H]2C(=O)NCc2ccc(-c3scnc3C)cc2OCCCCCCNC(=O)c2nc(N3CCc4cccc(C(=O)Nc5nc6ccccc6s5)c4C3)ccc2-c2cnn(CC3CCCCC3)c2C)C(C)C)on1] |
| I-199 | 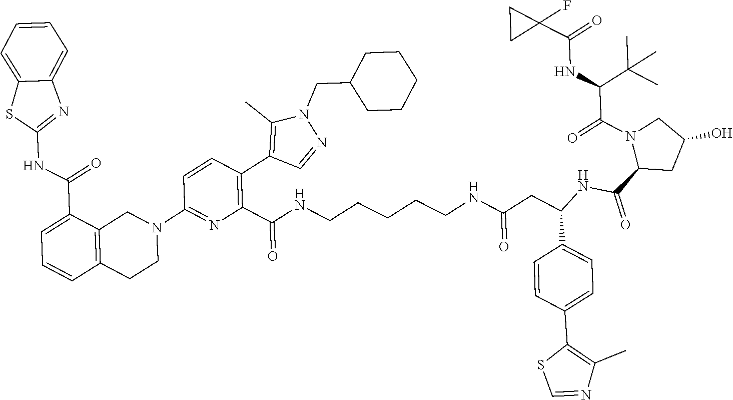 [SMILES: Cc1ncsc1-c1ccc([C@H](CC(=O)NCCCCCNC(=O)c2nc(N3CCc4cccc(C(=O)Nc5nc6ccccc6s5)c4C3)ccc2-c2cnn(CC3CCCCC3)c2C)NC(=O)[C@@H]2C[C@@H](O)CN2C(=O)[C@@H](NC(=O)C2(F)CC2)C(C)(C)C)cc1] |
| I-200 | 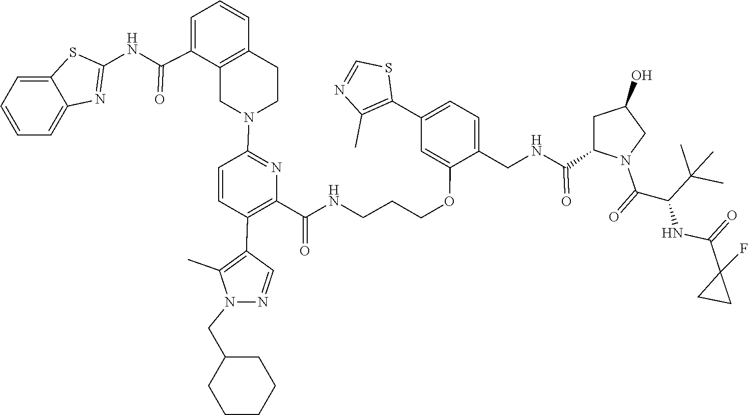 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCNC(=O)c2nc(N3CCc4cccc(C(=O)Nc5nc6ccccc6s5)c4C3)ccc2-c2cnn(CC3CCCCC3)c2C)c1] |
| I-201 | 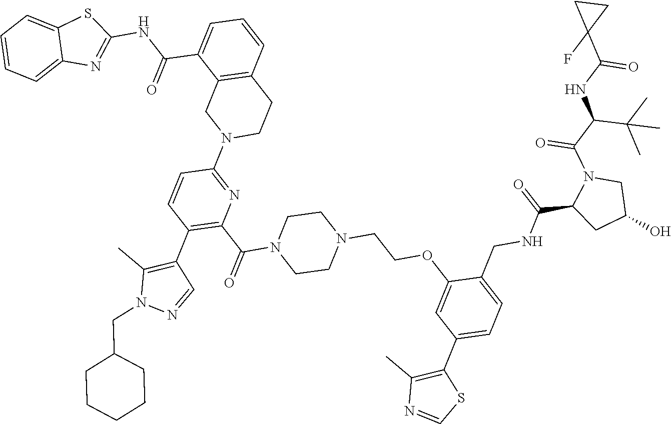 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(C(=O)c3nc(N4CCc5cccc(C(=O)Nc6nc7ccccc7s6)c5C4)ccc3-c3cnn(CC4CCCCC4)c3C)CC2)c1] |
| I-202 | 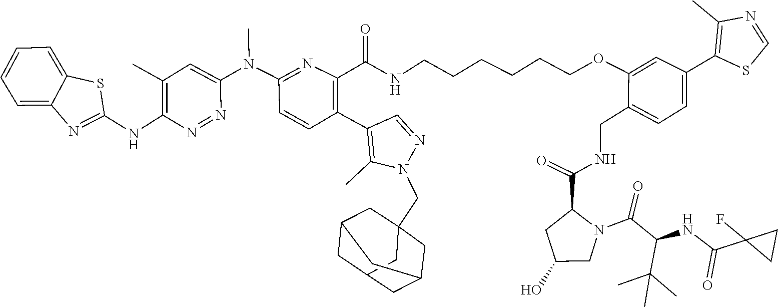 [SMILES: CCC1(Cn2ncc(-c3ccc(N(C)c4cc(C)c(Nc5nc6ccccc6s5)nn4)nc3C(=O)NCCCCCCOc3cc(-c4scnc4C)ccc3CNC(=O)[C@@H]3C[C@@H](O)CN3C(=O)[C@@H](NC(=O)C3(F)CC3)C(C)(C)C)c2C)CC2CC(C)CC(C2)C1] |
| I-203 | 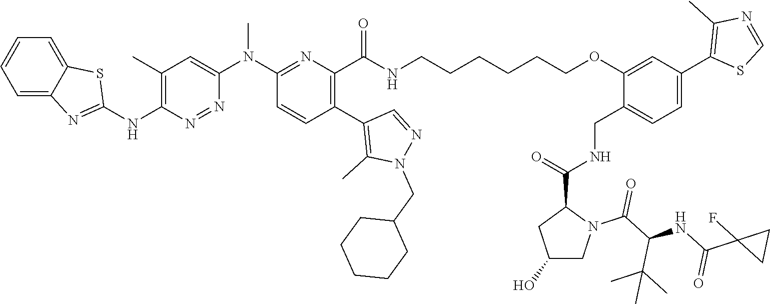 [SMILES: Cc1cc(N(C)c2ccc(-c3cnn(CC4CCCCC4)c3C)c(C(=O)NCCCCCCOc3cc(-c4scnc4C)ccc3CNC(=O)[C@@H]3C[C@@H](O)CN3C(=O)[C@@H](NC(=O)C3(F)CC3)C(C)(C)C)n2)nnc1Nc1nc2ccccc2s1] |
| I-204 | 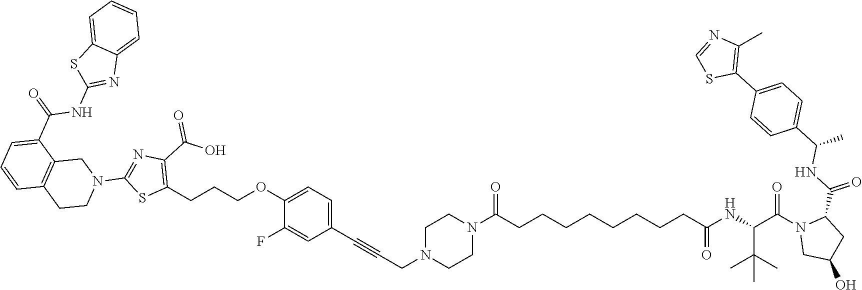 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CC#Cc3ccc(OCCCc4sc(N5CCc6cccc(C(=O)Nc7nc8ccccc8s7)c6C5)nc4C(=O)O)c(F)c3)CC2)C(C)(C)C)cc1] |
| I-205 | 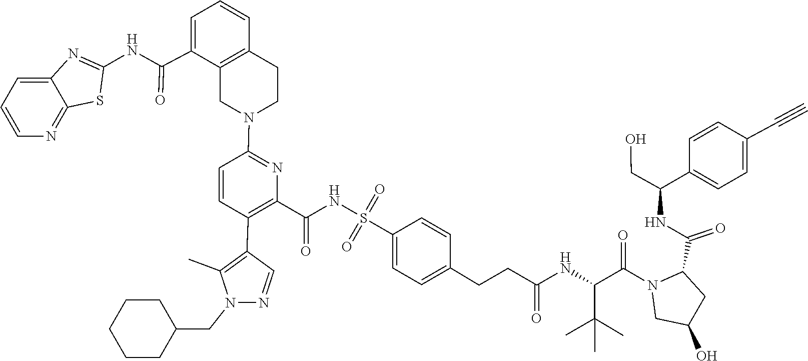 [SMILES: C#Cc1ccc([C@H](CO)NC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7cccnc7s6)c5C4)ccc3-c3cnn(CC4CCCCC4)c3C)cc2)C(C)(C)C)cc1] |
| I-206 | 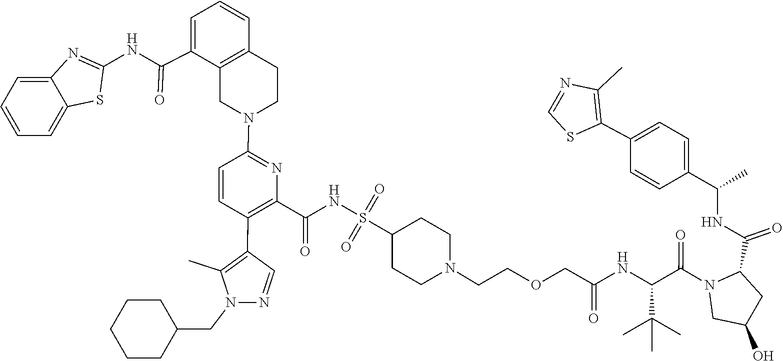 [SMILES: Cc1ncsc1-c1ccc([C@H](C)NC(=O)[C@@H]2C[C@@H](O)CN2C(=O)[C@@H](NC(=O)COCCN2CCC(S(=O)(=O)NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-207 | 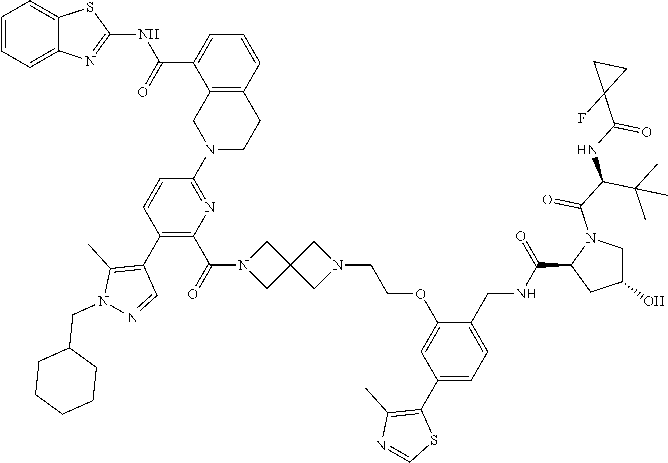 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CC3(C2)CN(C(=O)c2nc(N4CCc5cccc(C(=O)Nc6nc7ccccc7s6)c5C4)ccc2-c2cnn(CC4CCCCC4)c2C)C3)c1] |
| I-208 | 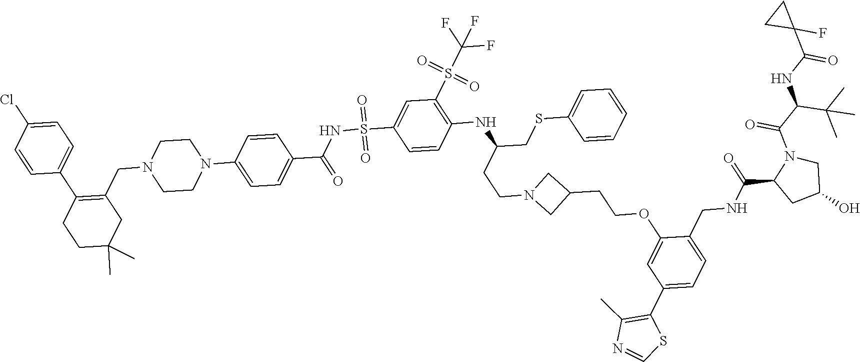 [SMILES: Cc1ncsc1-c1ccc(CNC(=O)[C@@H]2C[C@@H](O)CN2C(=O)[C@@H](NC(=O)C2(F)CC2)C(C)(C)C)c(OCCC2CN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)C2)c1] |
| I-209 | 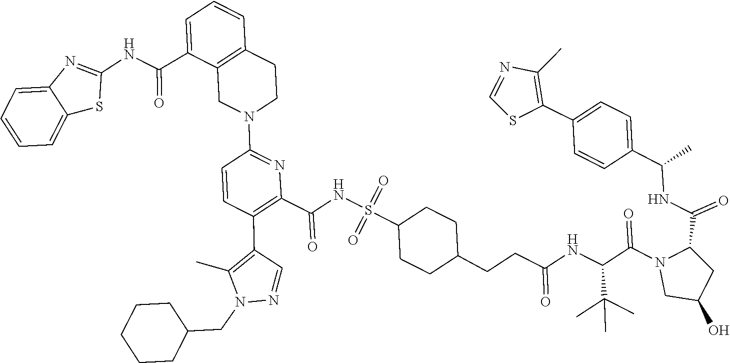 [SMILES: Cc1ncsc1-c1ccc([C@H](C)NC(=O)[C@@H]2C[C@@H](O)CN2C(=O)[C@@H](NC(=O)CCC2CCC(S(=O)(=O)NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-210 | 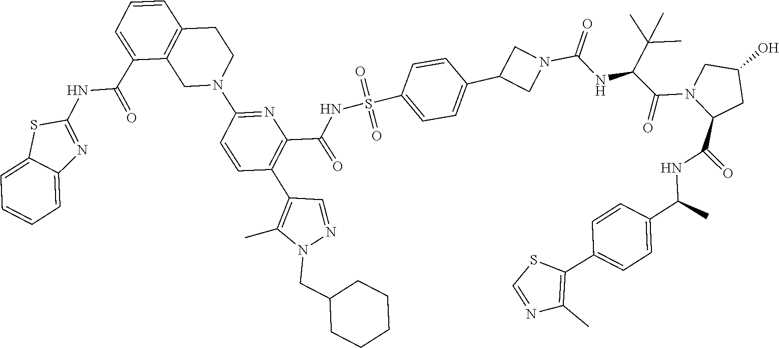 [SMILES: Cc1ncsc1-c1ccc([C@H](C)NC(=O)[C@@H]2C[C@@H](O)CN2C(=O)[C@@H](NC(=O)N2CC(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C2)C(C)(C)C)cc1] |
| I-211 | 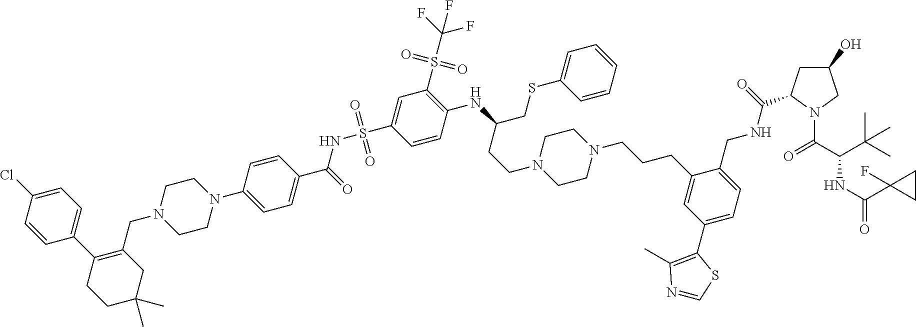 [SMILES: Cc1ncsc1-c1ccc(CNC(=O)[C@@H]2C[C@@H](O)CN2C(=O)[C@@H](NC(=O)C2(F)CC2)C(C)(C)C)c(CCCN2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-212 | 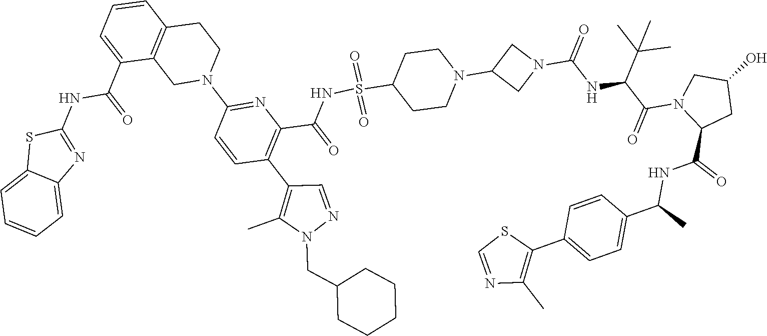 [SMILES: Cc1ncsc1-c1ccc([C@H](C)NC(=O)[C@@H]2C[C@@H](O)CN2C(=O)[C@@H](NC(=O)N2CC(N3CCC(S(=O)(=O)NC(=O)c4nc(N5CCc6cccc(C(=O)Nc7nc8ccccc8s7)c6C5)ccc4-c4cnn(CC5CCCCC5)c4C)CC3)C2)C(C)(C)C)cc1] |
| I-213 | 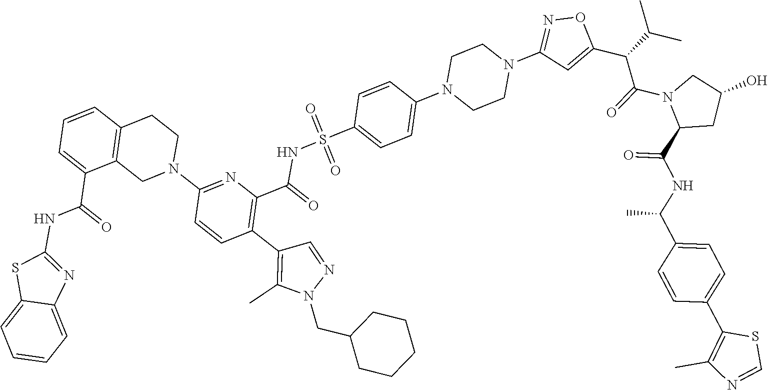 [SMILES: Cc1ncsc1-c1ccc([C@H](C)NC(=O)[C@@H]2C[C@@H](O)CN2C(=O)[C@H](c2cc(N3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)cc1] |
| I-214 | 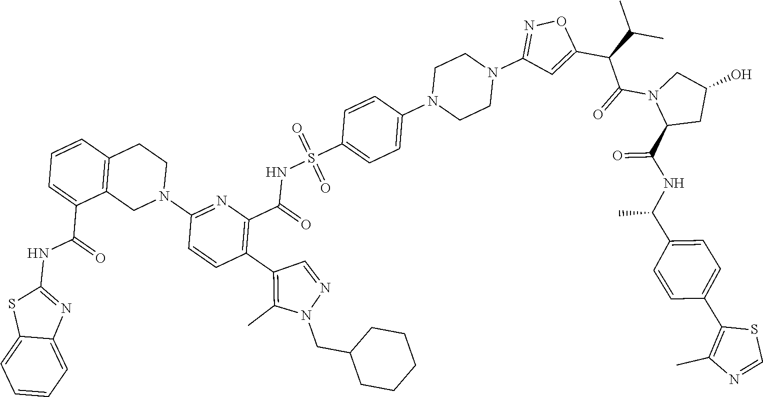 [SMILES: Cc1ncsc1-c1ccc([C@H](C)NC(=O)[C@@H]2C[C@@H](O)CN2C(=O)[C@@H](c2cc(N3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)cc1] |
| I-215 | 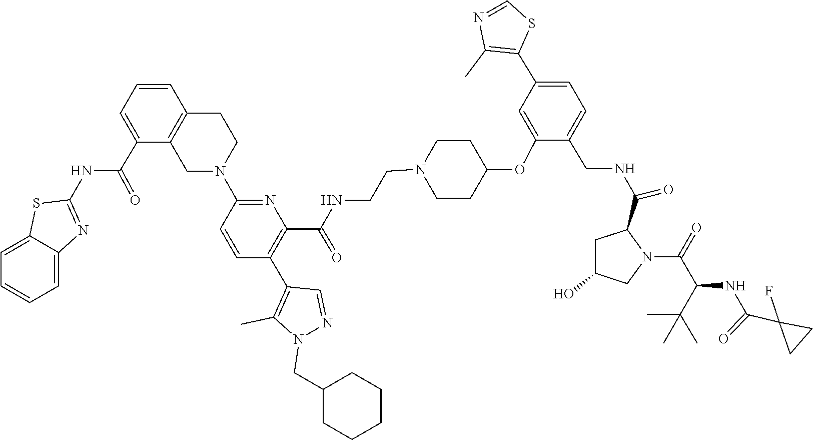 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CCN(CCNC(=O)c3nc(N4CCc5cccc(C(=O)Nc6nc7ccccc7s6)c5C4)ccc3-c3cnn(CC4CCCCC4)c3C)CC2)c1] |
| I-216 | 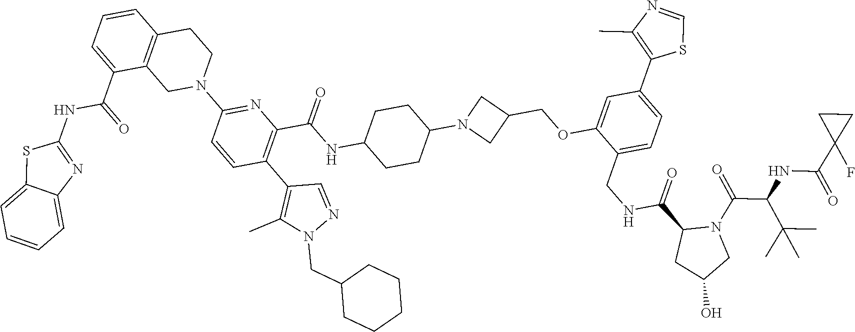 [SMILES: Cc1ncsc1-c1ccc(CNC(=O)[C@@H]2C[C@@H](O)CN2C(=O)[C@@H](NC(=O)C2(F)CC2)C(C)(C)C)c(OCC2CN(C3CCC(NC(=O)c4nc(N5CCc6cccc(C(=O)Nc7nc8ccccc8s7)c6C5)ccc4-c4cnn(CC5CCCCC5)c4C)CC3)C2)c1] |
| I-217 | 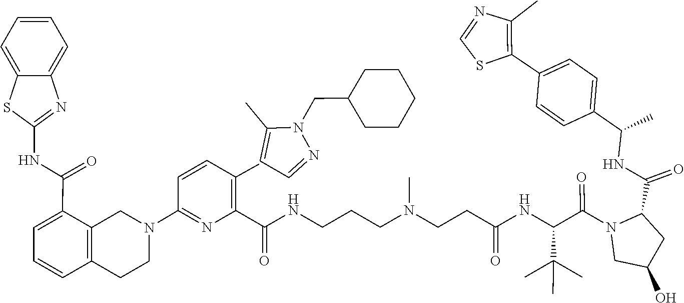 |
| I-218 | 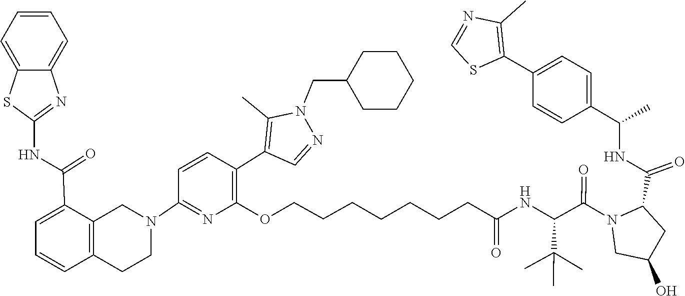 [SMILES: Cc1ncsc1-c1ccc([C@H](C)NC(=O)[C@@H]2C[C@@H](O)CN2C(=O)[C@@H](NC(=O)CCCCCCCOc2nc(N3CCc4cccc(C(=O)Nc5nc6ccccc6s5)c4C3)ccc2-c2cnn(CC3CCCCC3)c2C)C(C)(C)C)cc1] |
| I-219 | 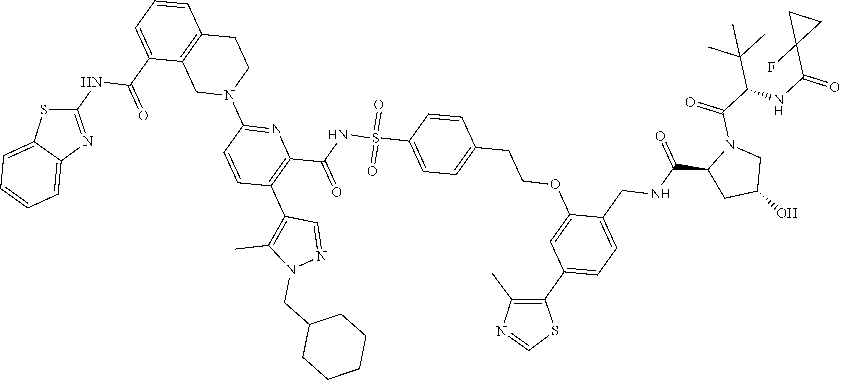 [SMILES: Cc1ncsc1-c1ccc(CNC(=O)[C@@H]2C[C@@H](O)CN2C(=O)[C@@H](NC(=O)C2(F)CC2)C(C)(C)C)c(OCCc2ccc(S(=O)(=O)NC(=O)c3nc(N4CCc5cccc(C(=O)Nc6nc7ccccc7s6)c5C4)ccc3-c3cnn(CC4CCCCC4)c3C)cc2)c1] |
| I-220 | 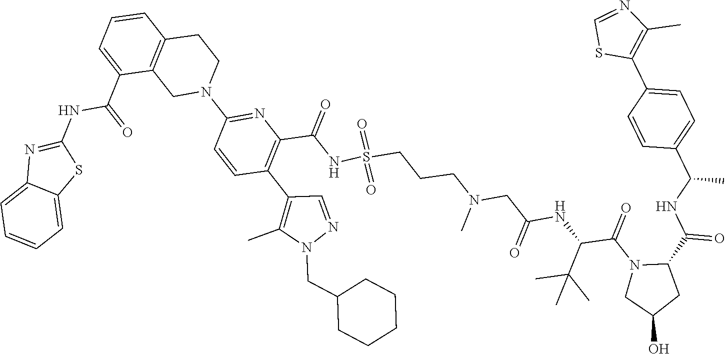 [SMILES: Cc1ncsc1-c1ccc([C@H](C)NC(=O)[C@@H]2C[C@@H](O)CN2C(=O)[C@@H](NC(=O)CN(C)CCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-221 | 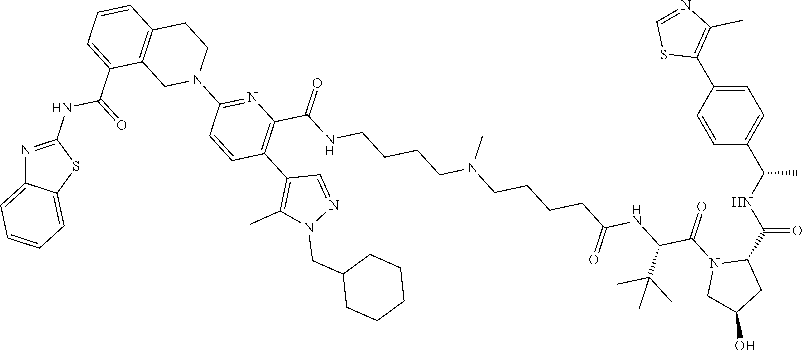 [SMILES: Cc1ncsc1-c1ccc([C@H](C)NC(=O)[C@@H]2C[C@@H](O)CN2C(=O)[C@@H](NC(=O)CCCCN(C)CCCCNC(=O)c2nc(N3CCc4cccc(C(=O)Nc5nc6ccccc6s5)c4C3)ccc2-c2cnn(CC3CCCCC3)c2C)C(C)(C)C)cc1] |
| I-222 | 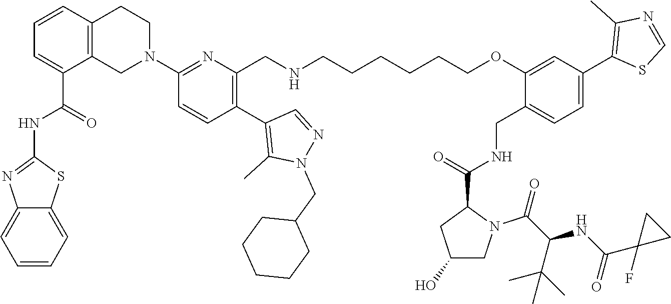 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCNCc2nc(N3CCc4cccc(C(=O)Nc5nc6ccccc6s5)c4C3)ccc2-c2cnn(CC3CCCCC3)c2C)c1] |
| I-223 | 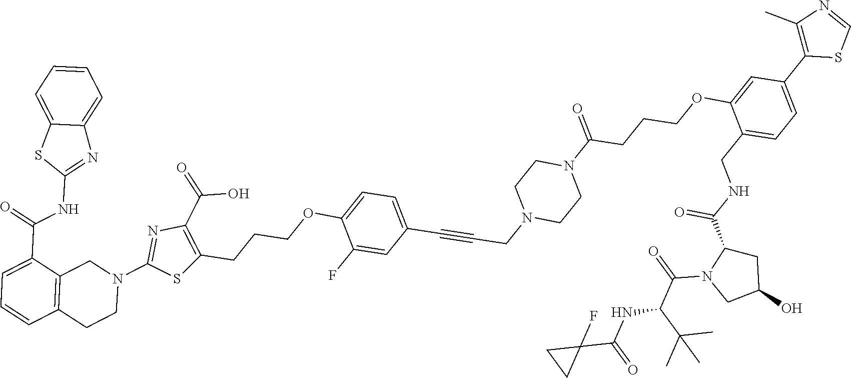 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CCN(CC#Cc3ccc(OCCCc4sc(N5CCc6cccc(C(=O)Nc7nc8ccccc8s7)c6C5)nc4C(=O)O)c(F)c3)CC2)c1] |
| I-224 | 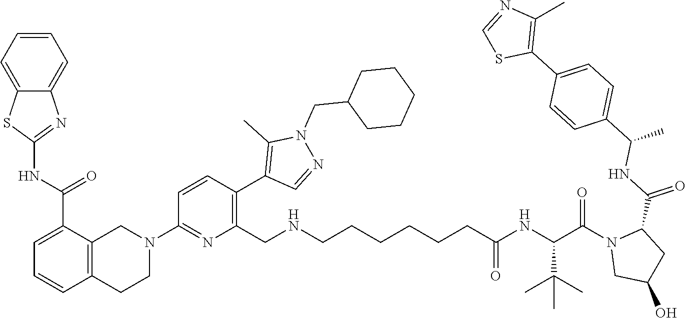 [SMILES: Cc1ncsc1-c1ccc([C@H](C)NC(=O)[C@@H]2C[C@@H](O)CN2C(=O)[C@@H](NC(=O)CCCCCCNCc2nc(N3CCc4cccc(C(=O)Nc5nc6ccccc6s5)c4C3)ccc2-c2cnn(CC3CCCCC3)c2C)C(C)(C)C)cc1] |
| I-225 | 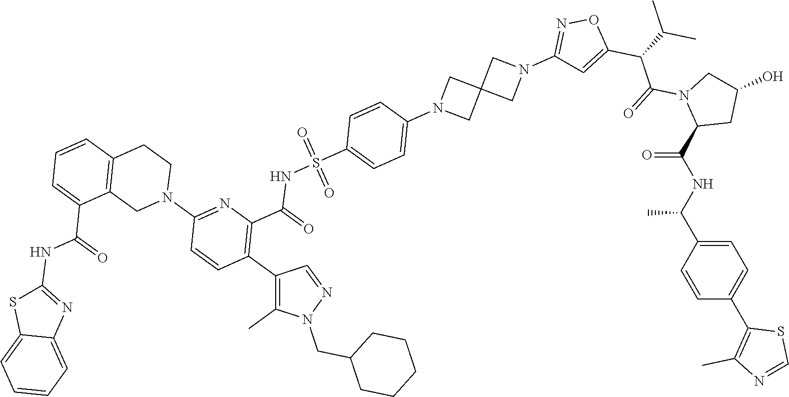 [SMILES: Cc1ncsc1-c1ccc([C@H](C)NC(=O)[C@@H]2C[C@@H](O)CN2C(=O)[C@H](c2cc(N3CC4(CN(c5ccc(S(=O)(=O)NC(=O)c6nc(N7CCc8cccc(C(=O)Nc9nc%10ccccc%10s9)c8C7)ccc6-c6cnn(CC7CCCCC7)c6C)cc5)C4)C3)no2)C(C)C)cc1] |
| I-226 | 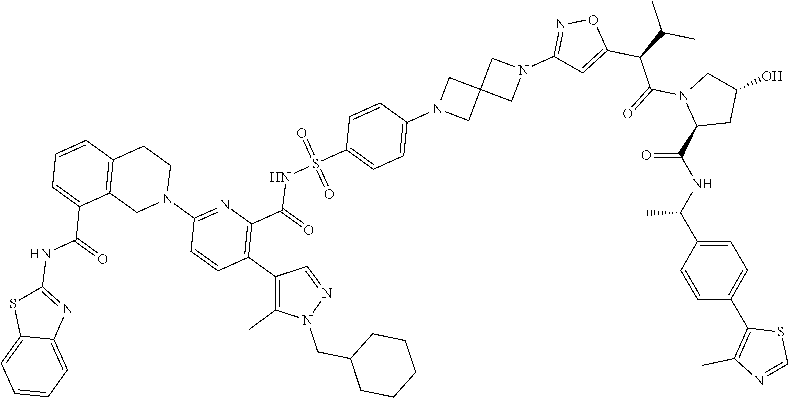 [SMILES: Cc1ncsc1-c1ccc([C@H](C)NC(=O)[C@@H]2C[C@@H](O)CN2C(=O)[C@@H](c2cc(N3CC4(CN(c5ccc(S(=O)(=O)NC(=O)c6nc(N7CCc8cccc(C(=O)Nc9nc%10ccccc%10s9)c8C7)ccc6-c6cnn(CC7CCCCC7)c6C)cc5)C4)C3)no2)C(C)C)cc1] |
| I-227 | 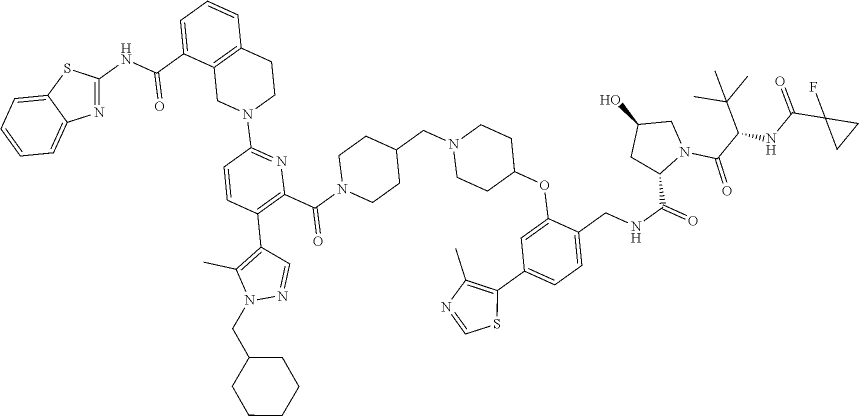 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CCN(CC3CCN(C(=O)c4nc(N5CCc6cccc(C(=O)Nc7nc8ccccc8s7)c6C5)ccc4-c4cnn(CC5CCCCC5)c4C)CC3)CC2)c1] |
| I-228 | 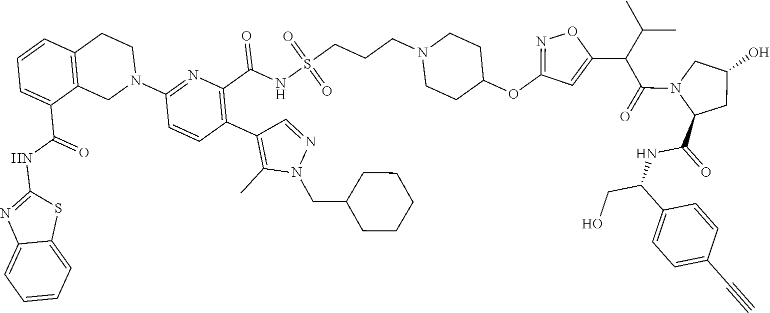 [SMILES: C#Cc1ccc([C@H](CO)NC(=O)[C@@H]2C[C@@H](O)CN2C(=O)C(c2cc(OC3CCN(CCCS(=O)(=O)NC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-229 | 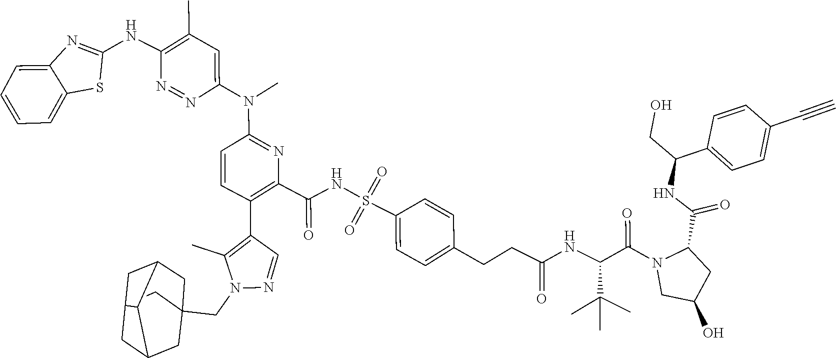 |
| I-230 | 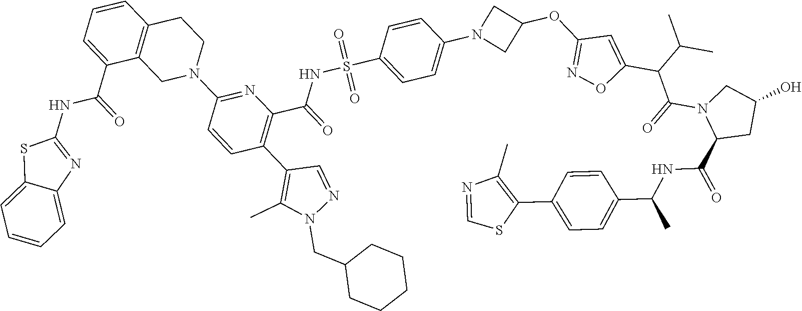 [SMILES: Cc1ncsc1-c1ccc([C@H](C)NC(=O)[C@@H]2C[C@@H](O)CN2C(=O)C(c2cc(OC3CN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)C3)no2)C(C)C)cc1] |
| I-231 | 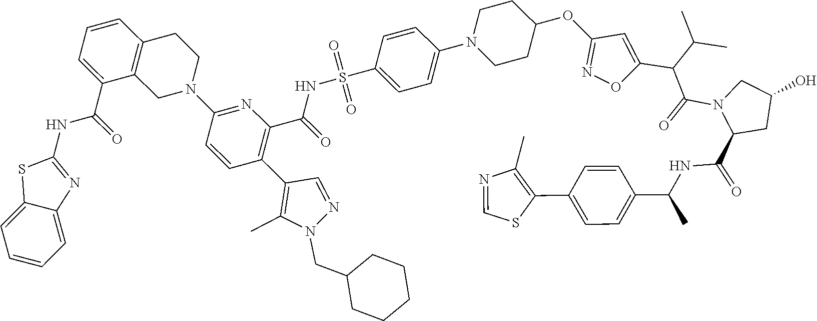 [SMILES: Cc1ncsc1-c1ccc([C@H](C)NC(=O)[C@@H]2C[C@@H](O)CN2C(=O)C(c2cc(OC3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)cc1] |
| I-232 | 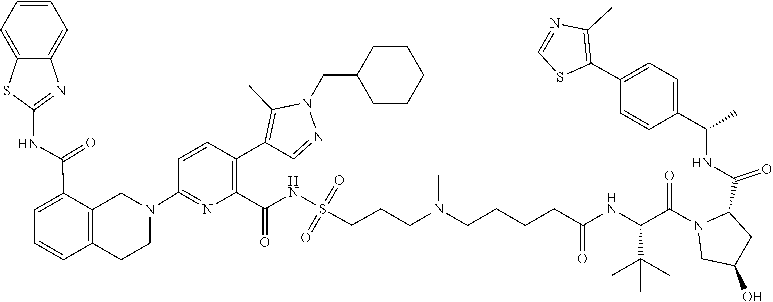 [SMILES: Cc1ncsc1-c1ccc([C@H](C)NC(=O)[C@@H]2C[C@@H](O)CN2C(=O)[C@@H](NC(=O)CCCCN(C)CCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-233 | 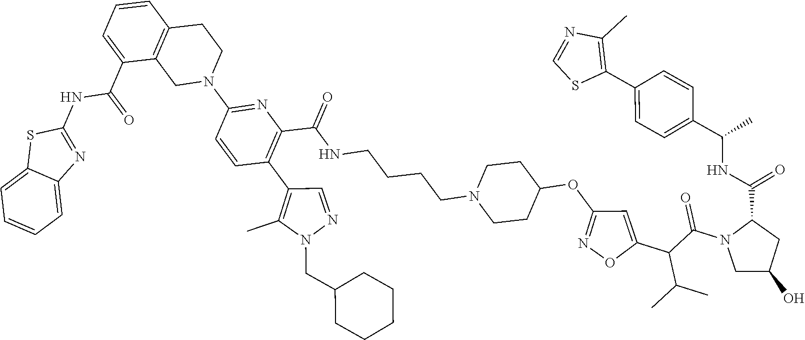 [SMILES: Cc1ncsc1-c1ccc([C@H](C)NC(=O)[C@@H]2C[C@@H](O)CN2C(=O)C(c2cc(OC3CCN(CCCCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-234 | 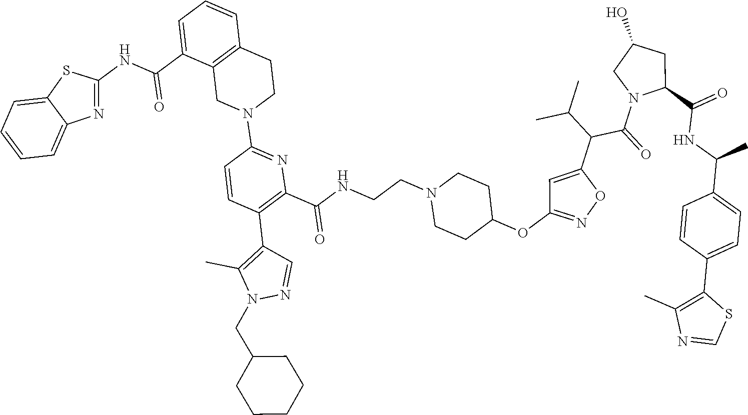 [SMILES: Cc1ncsc1-c1ccc([C@H](C)NC(=O)[C@@H]2C[C@@H](O)CN2C(=O)C(c2cc(OC3CCN(CCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-235 | 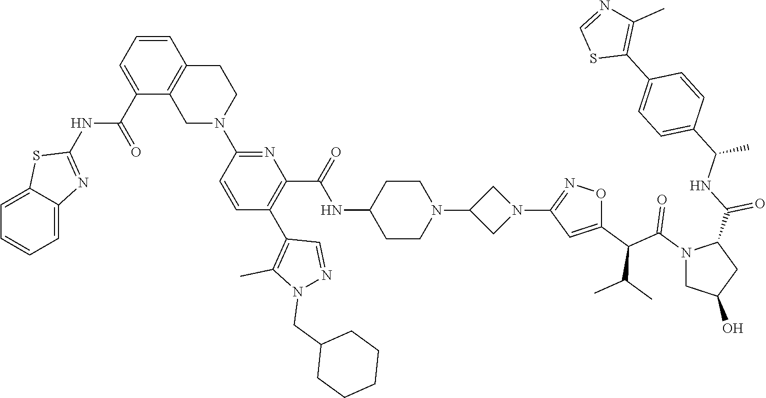 [SMILES: Cc1ncsc1-c1ccc([C@H](C)NC(=O)[C@@H]2C[C@@H](O)CN2C(=O)[C@H](c2cc(N3CC(N4CCC(NC(=O)c5nc(N6CCc7cccc(C(=O)Nc8nc9ccccc9s8)c7C6)ccc5-c5cnn(CC6CCCCC6)c5C)CC4)C3)no2)C(C)C)cc1] |
| I-236 | 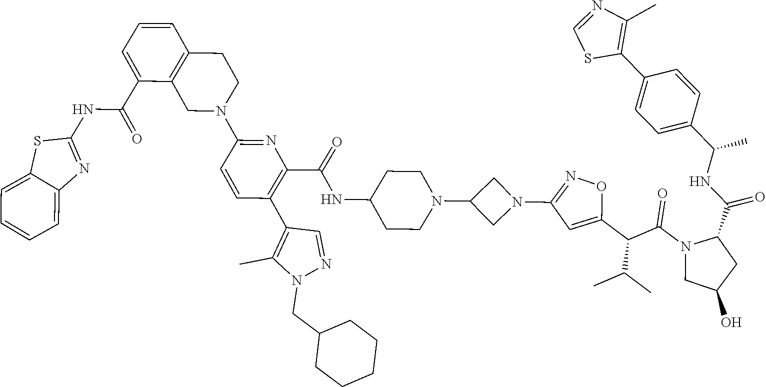 [SMILES: Cc1ncsc1-c1ccc([C@H](C)NC(=O)[C@@H]2C[C@@H](O)CN2C(=O)[C@@H](c2cc(N3CC(N4CCC(NC(=O)c5nc(N6CCc7cccc(C(=O)Nc8nc9ccccc9s8)c7C6)ccc5-c5cnn(CC6CCCCC6)c5C)CC4)C3)no2)C(C)C)cc1] |
| I-237 | 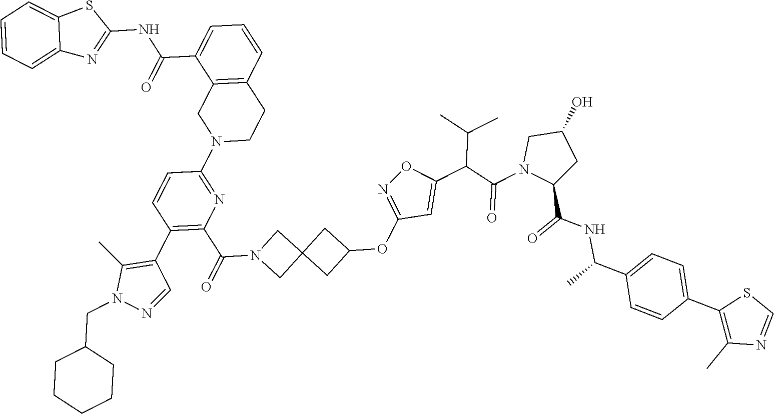 [SMILES: Cc1ncsc1-c1ccc([C@H](C)NC(=O)[C@@H]2C[C@@H](O)CN2C(=O)C(c2cc(OC3CC4(C3)CN(C(=O)c3nc(N5CCc6cccc(C(=O)Nc7nc8ccccc8s7)c6C5)ccc3-c3cnn(CC5CCCCC5)c3C)C4)no2)C(C)C)cc1] |
| I-238 | 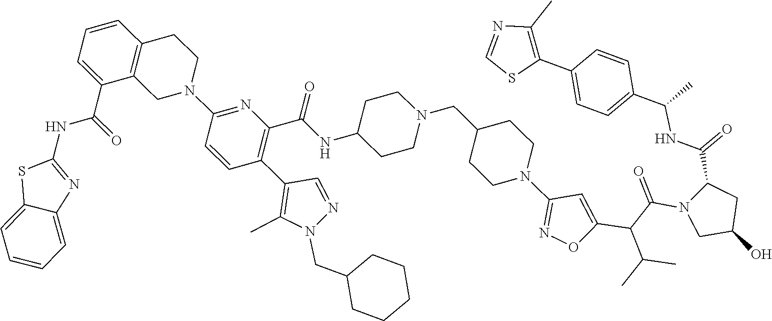 [SMILES: Cc1ncsc1-c1ccc([C@H](C)NC(=O)[C@@H]2C[C@@H](O)CN2C(=O)C(c2cc(N3CCC(CN4CCC(NC(=O)c5nc(N6CCc7cccc(C(=O)Nc8nc9ccccc9s8)c7C6)ccc5-c5cnn(CC6CCCCC6)c5C)CC4)CC3)no2)C(C)C)cc1] |
| I-239 | 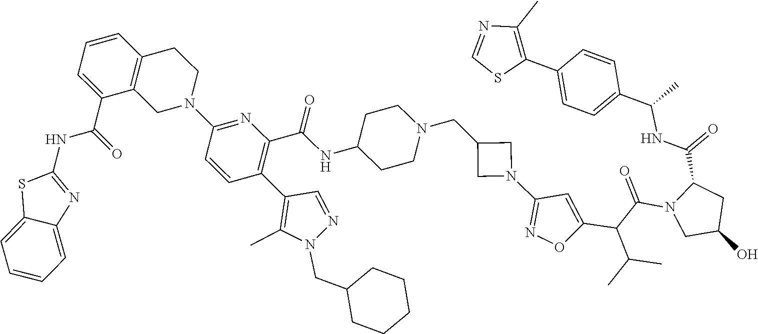 [SMILES: Cc1ncsc1-c1ccc([C@H](C)NC(=O)[C@@H]2C[C@@H](O)CN2C(=O)C(c2cc(N3CC(CN4CCC(NC(=O)c5nc(N6CCc7cccc(C(=O)Nc8nc9ccccc9s8)c7C6)ccc5-c5cnn(CC6CCCCC6)c5C)CC4)C3)no2)C(C)C)cc1] |
| I-240 | 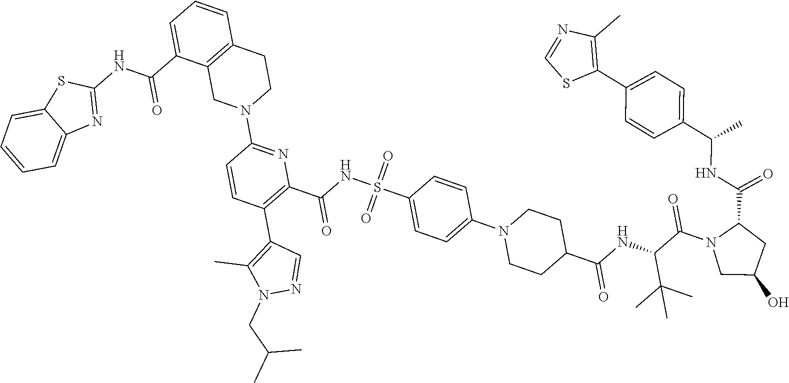 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC(C)C)c4C)cc3)CC2)C(C)(C)C)cc1] |
| I-241 | 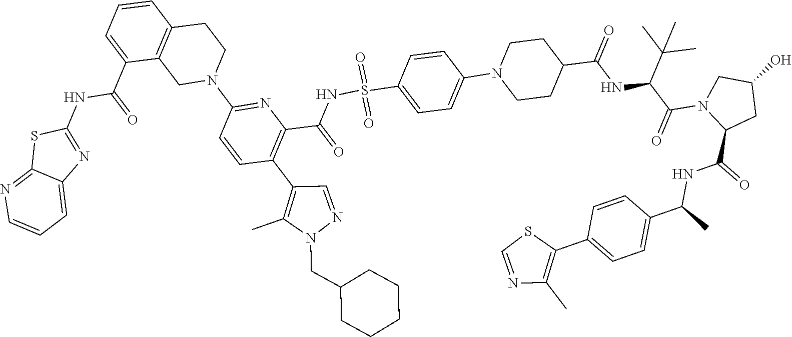 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8cccnc8s7)c6C5)ccc4-c4cnn(CC5CCCCC5)c4C)cc3)CC2)C(C)(C)C)cc1] |
| I-242 | 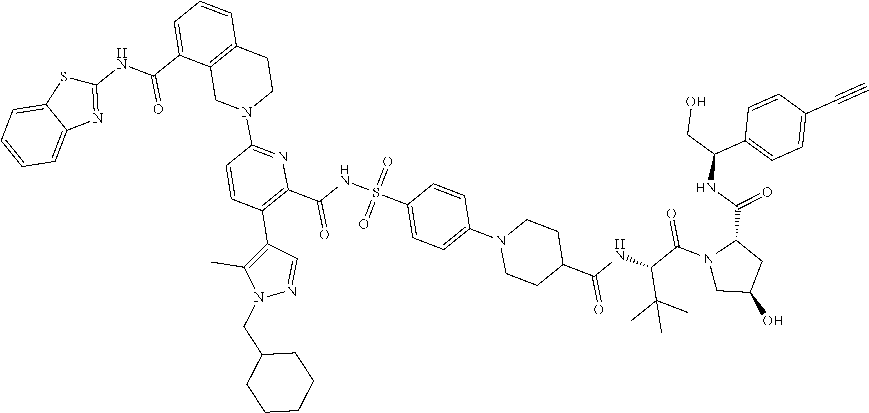 [SMILES: C#Cc1ccc([C@H](CO)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-243 | 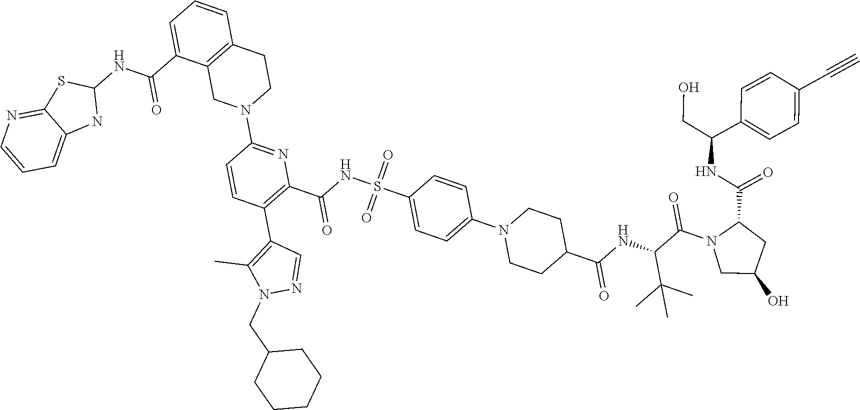 |
| I-244 | 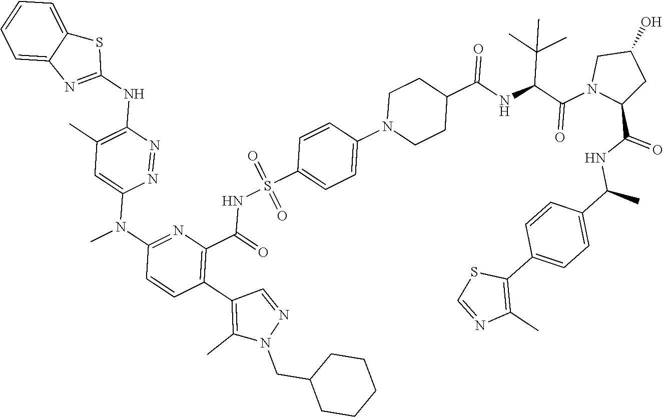 [SMILES: Cc1cc(N(C)c2ccc(-c3cnn(CC4CCCCC4)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2ccccc2s1] |
| I-245 | 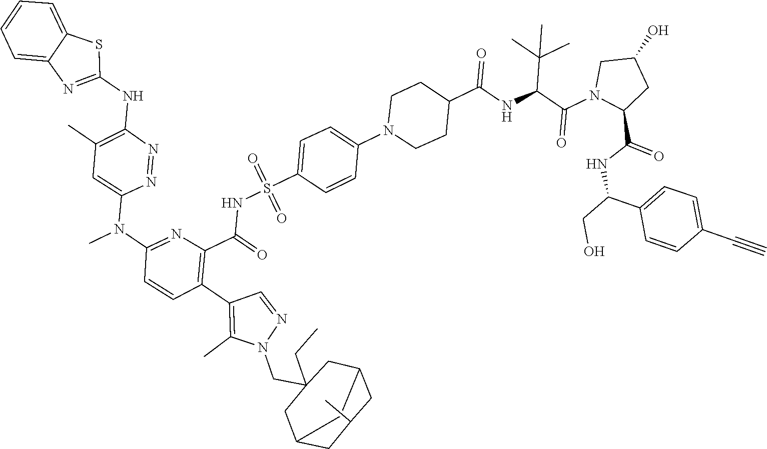 [SMILES: C#Cc1ccc([C@H](CO)NC(=O)[C@@H]2C[C@@H](O)CN2C(=O)[C@@H](NC(=O)C2CCN(c3ccc(S(=O)(=O)NC(=O)c4nc(N(C)c5cc(C)c(Nc6nc7ccccc7s6)nn5)ccc4-c4cnn(CC5(CC)CC6CC(C)CC(C6)C5)c4C)cc3)CC2)C(C)(C)C)cc1] |
| I-246 | 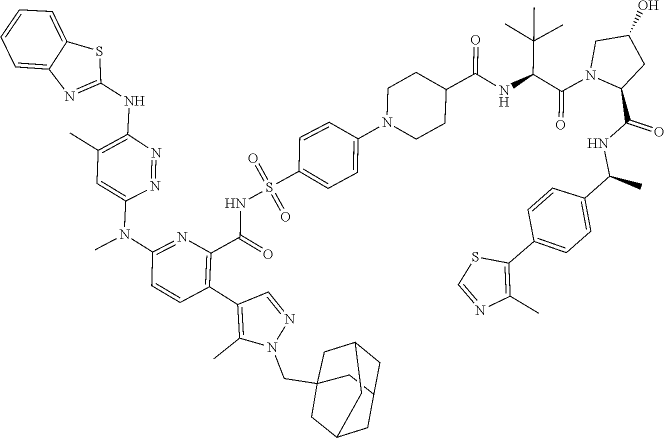 [SMILES: Cc1cc(N(C)c2ccc(-c3cnn(CC45CC6CC(CC(C6)C4)C5)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2ccccc2s1] |
| I-247 | 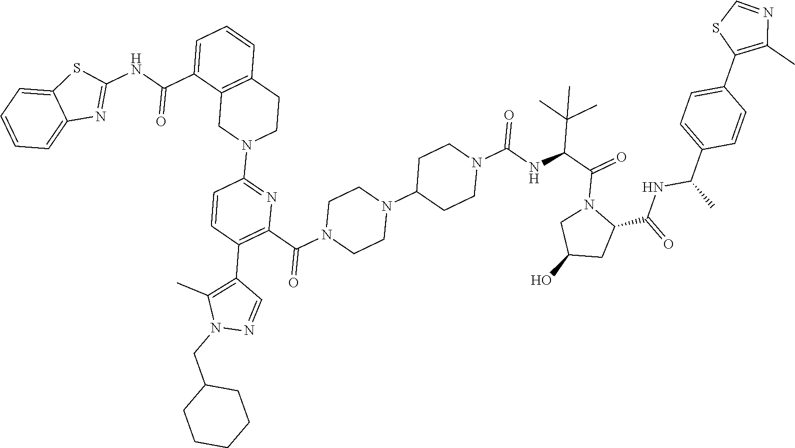 [SMILES: Cc1ncsc1-c1ccc([C@H](C)NC(=O)[C@@H]2C[C@@H](O)CN2C(=O)[C@@H](NC(=O)N2CCC(N3CCN(C(=O)c4nc(N5CCc6cccc(C(=O)Nc7nc8ccccc8s7)c6C5)ccc4-c4cnn(CC5CCCCC5)c4C)CC3)CC2)C(C)(C)C)cc1] |
| I-248 | 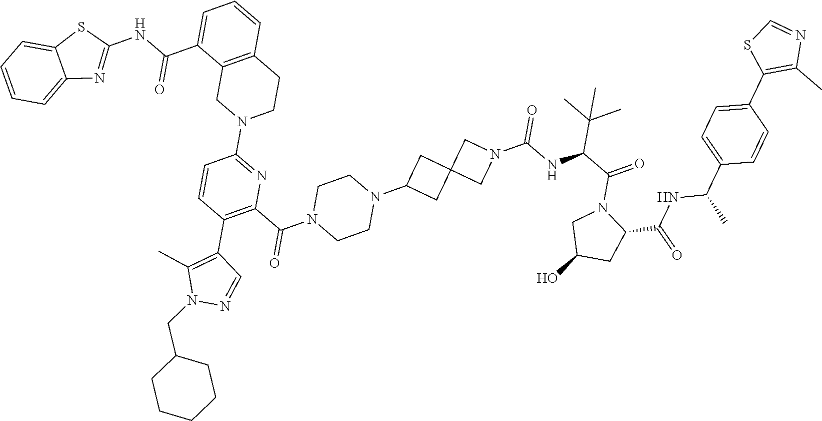 [SMILES: Cc1ncsc1-c1ccc([C@H](C)NC(=O)[C@@H]2C[C@@H](O)CN2C(=O)[C@@H](NC(=O)N2CC3(CC(N4CCN(C(=O)c5nc(N6CCc7cccc(C(=O)Nc8nc9ccccc9s8)c7C6)ccc5-c5cnn(CC6CCCCC6)c5C)CC4)C3)C2)C(C)(C)C)cc1] |
| I-249 | 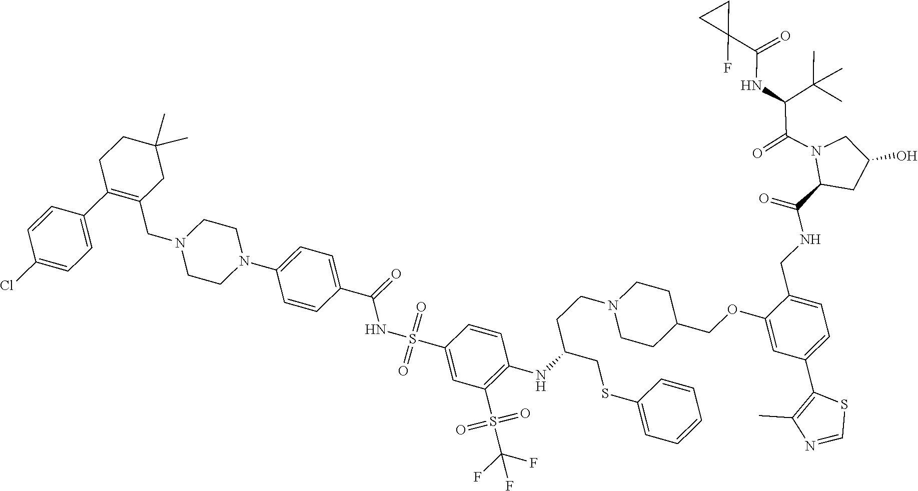 [SMILES: Cc1ncsc1-c1ccc(CNC(=O)[C@@H]2C[C@@H](O)CN2C(=O)[C@@H](NC(=O)C2(F)CC2)C(C)(C)C)c(OCC2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-250 | 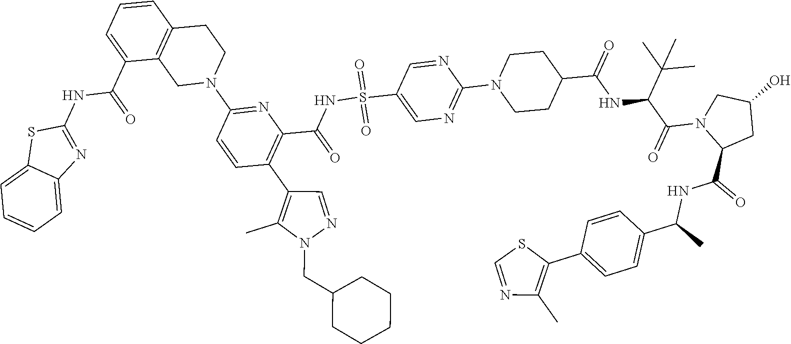 [SMILES: Cc1ncsc1-c1ccc([C@H](C)NC(=O)[C@@H]2C[C@@H](O)CN2C(=O)[C@@H](NC(=O)C2CCN(c3ncc(S(=O)(=O)NC(=O)c4nc(N5CCc6cccc(C(=O)Nc7nc8ccccc8s7)c6C5)ccc4-c4cnn(CC5CCCCC5)c4C)cn3)CC2)C(C)(C)C)cc1] |
| I-251 | 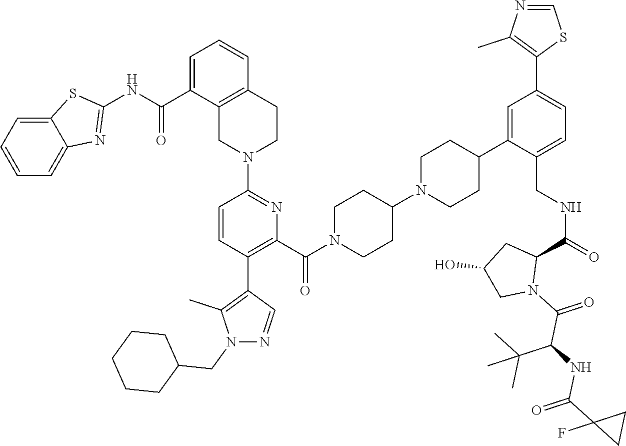 [SMILES: Cc1ncsc1-c1ccc(CNC(=O)[C@@H]2C[C@@H](O)CN2C(=O)[C@@H](NC(=O)C2(F)CC2)C(C)(C)C)c(C2CCN(C3CCN(C(=O)c4nc(N5CCc6cccc(C(=O)Nc7nc8ccccc8s7)c6C5)ccc4-c4cnn(CC5CCCCC5)c4C)CC3)CC2)c1] |
| I-252 | 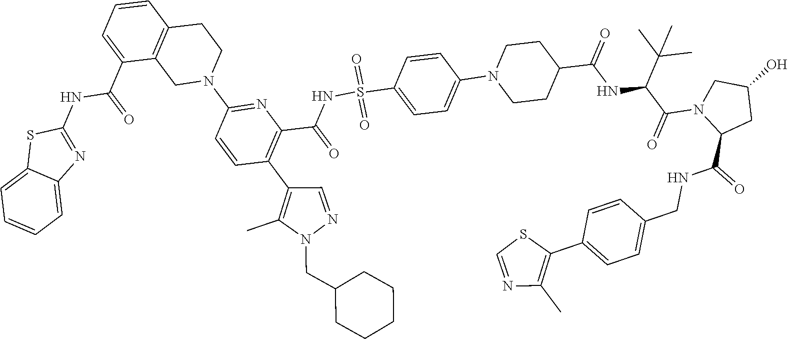 [SMILES: Cc1ncsc1-c1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-253 | 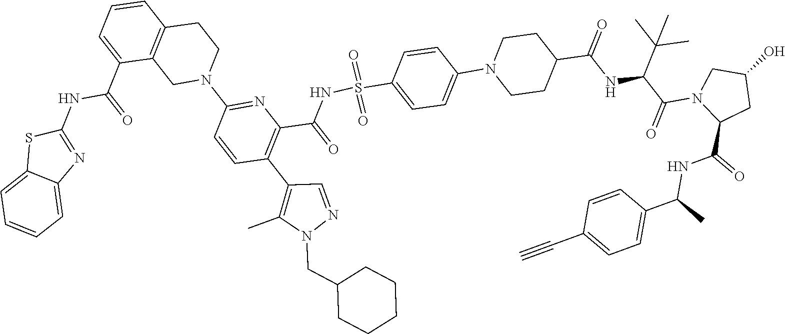 [SMILES: C#Cc1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-254 | 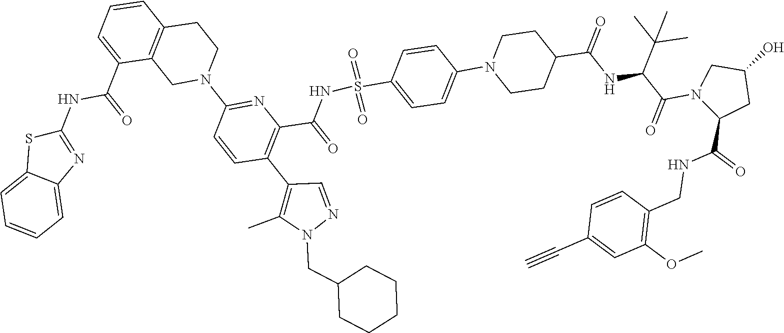 [SMILES: C#Cc1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)c(OC)c1] |
| I-255 | 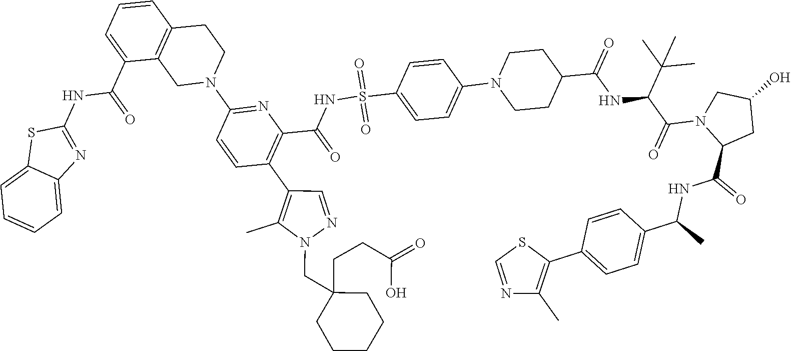 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5(CCC(=O)O)CCCCC5)c4C)cc3)CC2)C(C)(C)C)cc1] |
| I-256 | 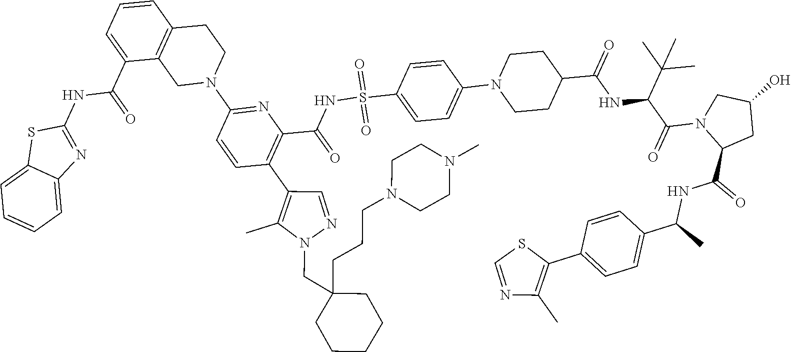 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5(CCCN6CCN(C)CC6)CCCCC5)c4C)cc3)CC2)C(C)(C)C)cc1] |
| I-257 | 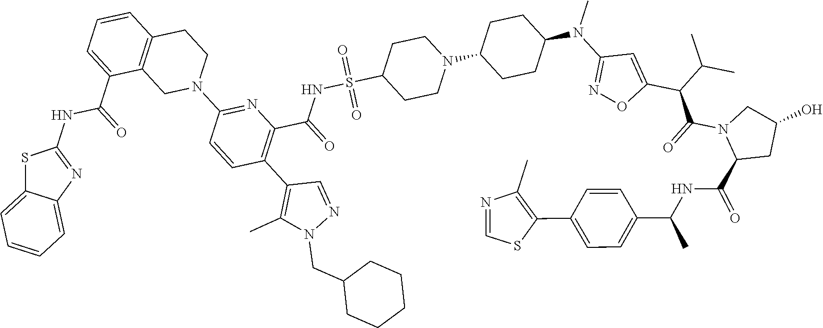 [SMILES: Cc1ncsc1-c1ccc([C@H](C)NC(=O)[C@@H]2C[C@@H](O)CN2C(=O)[C@@H](c2cc(N(C)[C@H]3CC[C@H](N4CCC(S(=O)(=O)NC(=O)c5nc(N6CCc7cccc(C(=O)Nc8nc9ccccc9s8)c7C6)ccc5-c5cnn(CC6CCCCC6)c5C)CC4)CC3)no2)C(C)C)cc1] |
| I-258 | 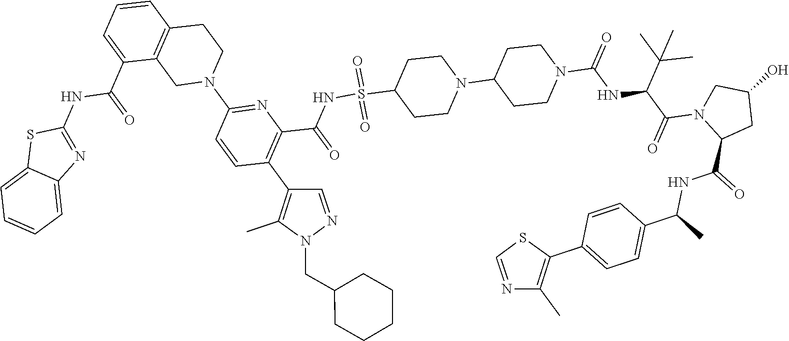 [SMILES: Cc1ncsc1-c1ccc([C@H](C)NC(=O)[C@@H]2C[C@@H](O)CN2C(=O)[C@@H](NC(=O)N2CCC(N3CCC(S(=O)(=O)NC(=O)c4nc(N5CCc6cccc(C(=O)Nc7nc8ccccc8s7)c6C5)ccc4-c4cnn(CC5CCCCC5)c4C)CC3)CC2)C(C)(C)C)cc1] |
| I-259 | 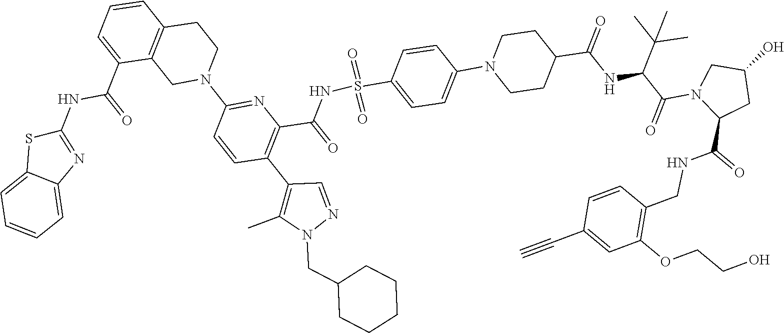 [SMILES: C#Cc1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)c(OCCO)c1] |
| I-260 | 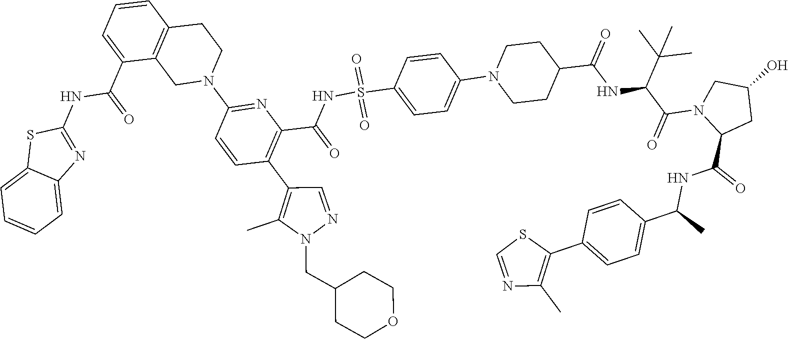 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCOCC5)c4C)cc3)CC2)C(C)(C)C)cc1] |
| I-261 | 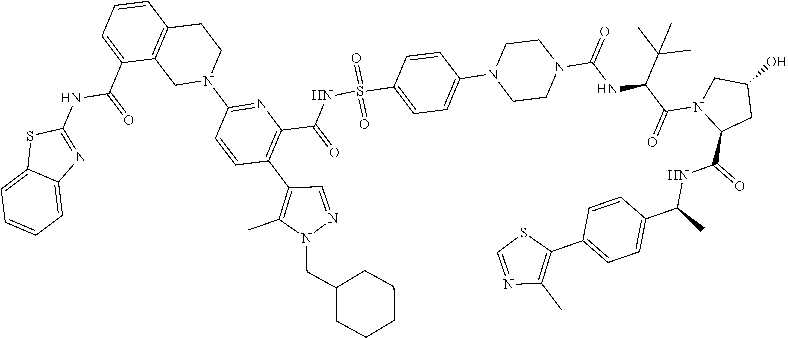 [SMILES: Cc1ncsc1-c1ccc([C@H](C)NC(=O)[C@@H]2C[C@@H](O)CN2C(=O)[C@@H](NC(=O)N2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-262 | 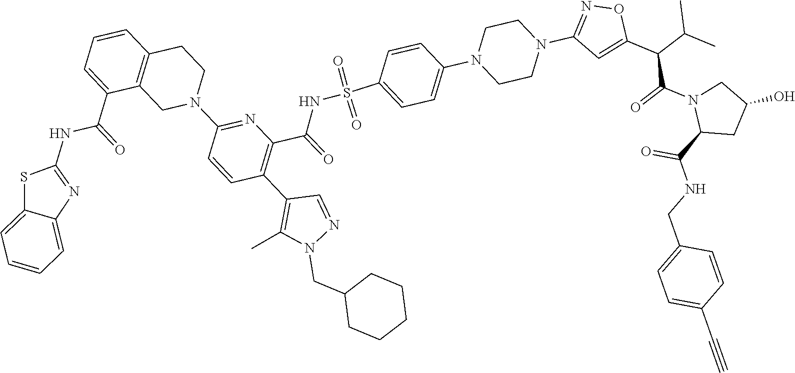 [SMILES: C#Cc1ccc(CNC(=O)[C@@H]2C[C@@H](O)CN2C(=O)[C@@H](c2cc(N3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)cc1] |
| I-263 | 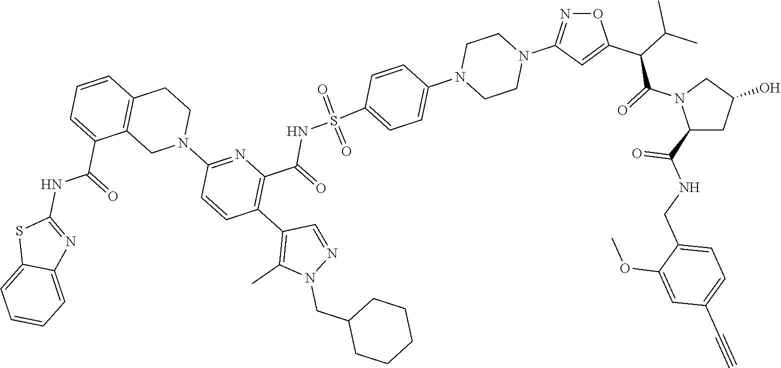 [SMILES: C#Cc1ccc(CNC(=O)[C@@H]2C[C@@H](O)CN2C(=O)[C@@H](c2cc(N3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)c(OC)c1] |
| I-264 | 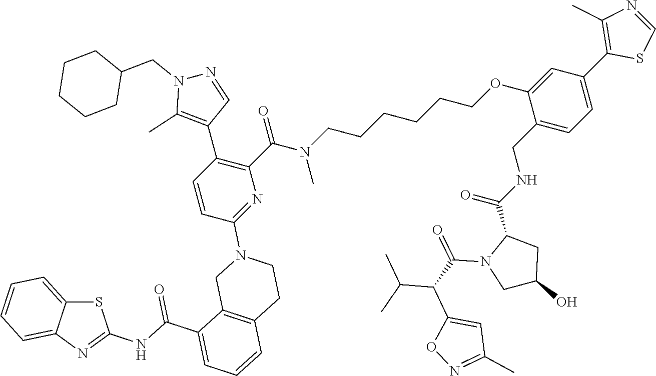 [SMILES: Cc1cc([C@@H](C(=O)N2C[C@H](O)C[C@H]2C(=O)NCc2ccc(-c3scnc3C)cc2OCCCCCCN(C)C(=O)c2nc(N3CCc4cccc(C(=O)Nc5nc6ccccc6s5)c4C3)ccc2-c2cnn(CC3CCCCC3)c2C)C(C)C)on1] |
| I-265 | 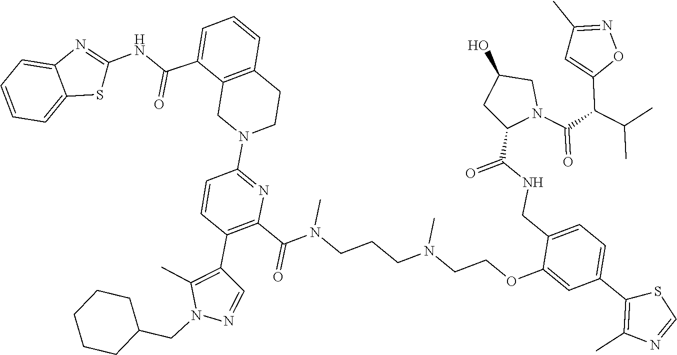 [SMILES: Cc1cc([C@@H](C(=O)N2C[C@H](O)C[C@H]2C(=O)NCc2ccc(-c3scnc3C)cc2OCCN(C)CCCN(C)C(=O)c2nc(N3CCc4cccc(C(=O)Nc5nc6ccccc6s5)c4C3)ccc2-c2cnn(CC3CCCCC3)c2C)C(C)C)on1] |
| I-266 | 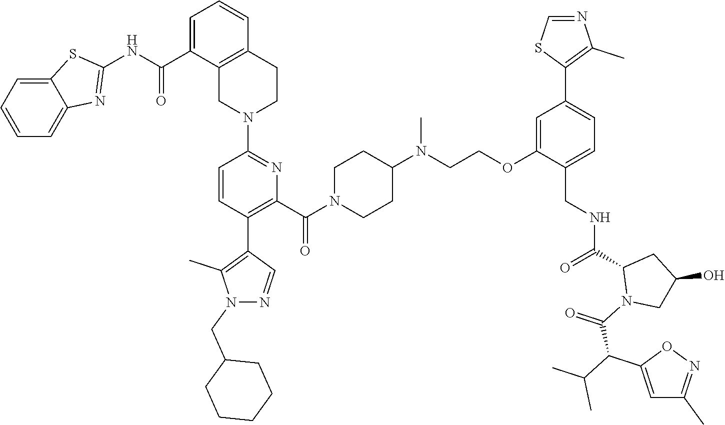 [SMILES: Cc1cc([C@@H](C(=O)N2C[C@H](O)C[C@H]2C(=O)NCc2ccc(-c3scnc3C)cc2OCCN(C)C2CCN(C(=O)c3nc(N4CCc5cccc(C(=O)Nc6nc7ccccc7s6)c5C4)ccc3-c3cnn(CC4CCCCC4)c3C)CC2)C(C)C)on1] |
| I-267 | 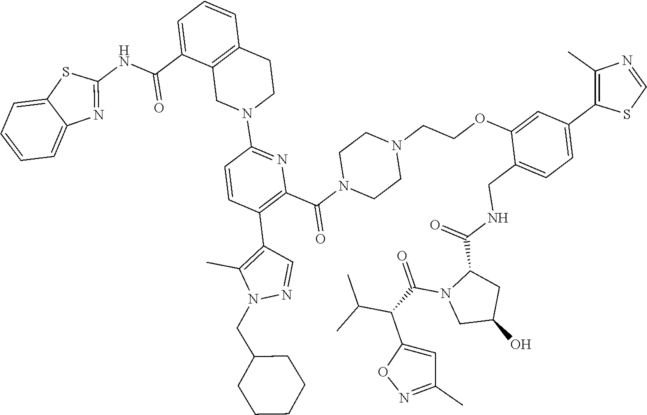 [SMILES: Cc1cc([C@@H](C(=O)N2C[C@H](O)C[C@H]2C(=O)NCc2ccc(-c3scnc3C)cc2OCCN2CCN(C(=O)c3nc(N4CCc5cccc(C(=O)Nc6nc7ccccc7s6)c5C4)ccc3-c3cnn(CC4CCCCC4)c3C)CC2)C(C)C)on1] |
| I-268 | 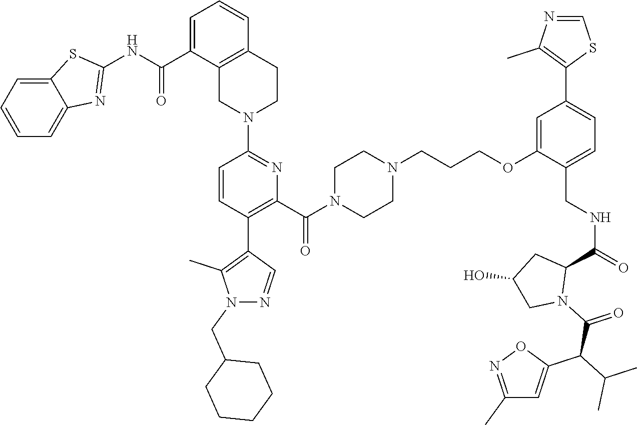 [SMILES: Cc1cc([C@@H](C(=O)N2C[C@H](O)C[C@H]2C(=O)NCc2ccc(-c3scnc3C)cc2OCCCN2CCN(C(=O)c3nc(N4CCc5cccc(C(=O)Nc6nc7ccccc7s6)c5C4)ccc3-c3cnn(CC4CCCCC4)c3C)CC2)C(C)C)on1] |
| I-269 | 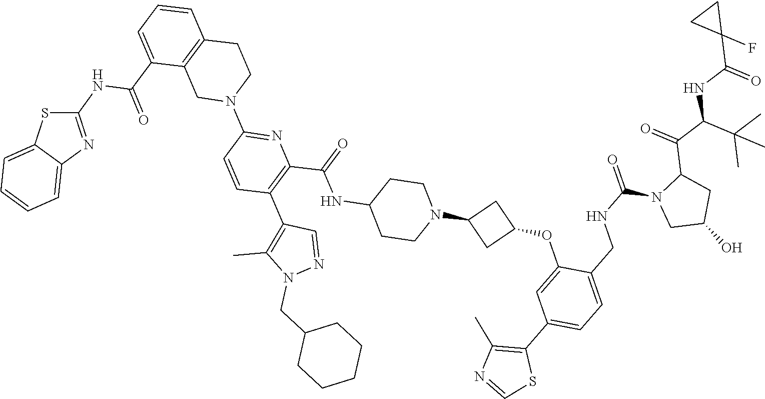 |
| I-270 | 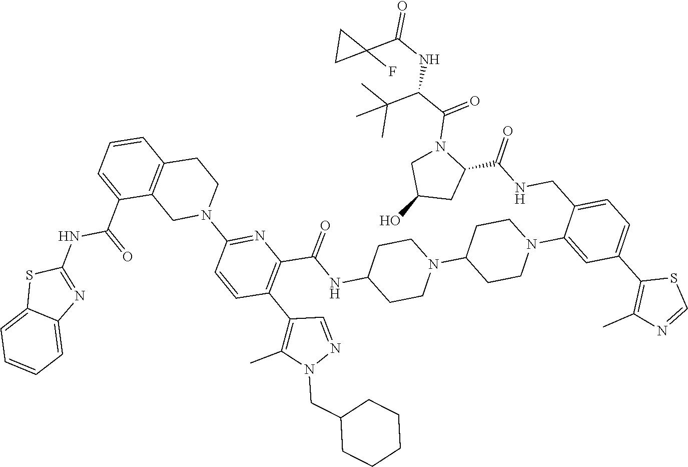 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(N3CCC(NC(=O)c4nc(N5CCc6cccc(C(=O)Nc7nc8ccccc8s7)c6C5)ccc4-c4cnn(CC5CCCCC5)c4C)CC3)CC2)c1] |
| I-271 | 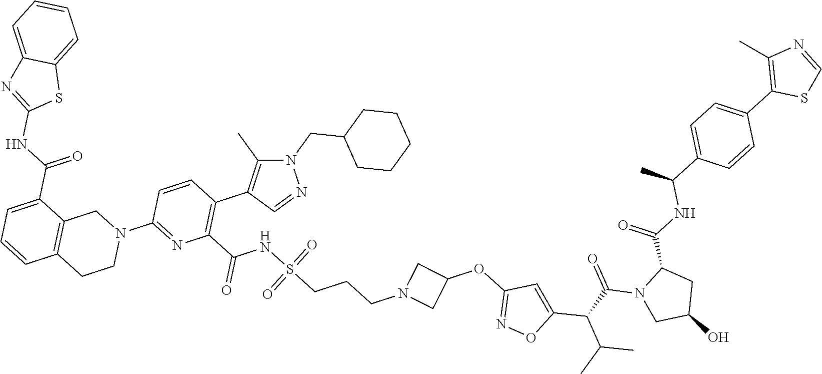 [SMILES: Cc1ncsc1-c1ccc([C@H](C)NC(=O)[C@@H]2C[C@@H](O)CN2C(=O)[C@@H](c2cc(OC3CN(CCCS(=O)(=O)NC(=O)c4nc(N5CCc6cccc(C(=O)Nc7nc8ccccc8s7)c6C5)ccc4-c4cnn(CC5CCCCC5)c4C)C3)no2)C(C)C)cc1] |
| I-272 | 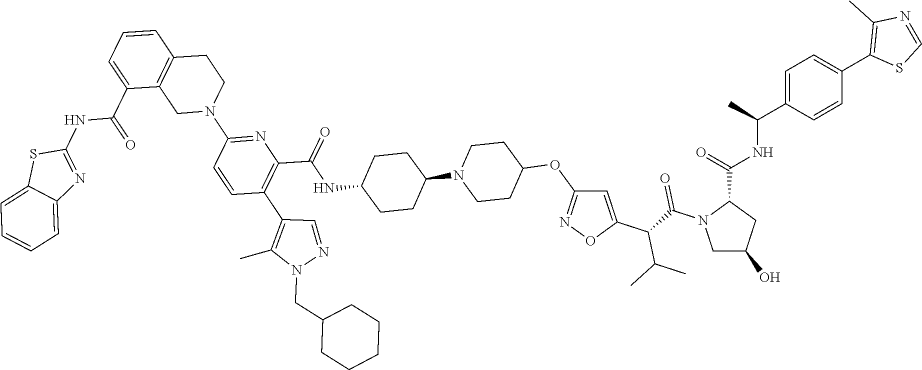 [SMILES: Cc1ncsc1-c1ccc([C@H](C)NC(=O)[C@@H]2C[C@@H](O)CN2C(=O)[C@@H](c2cc(OC3CCN([C@H]4CC[C@H](NC(=O)c5nc(N6CCc7cccc(C(=O)Nc8nc9ccccc9s8)c7C6)ccc5-c5cnn(CC6CCCCC6)c5C)CC4)CC3)no2)C(C)C)cc1] |
| I-273 | 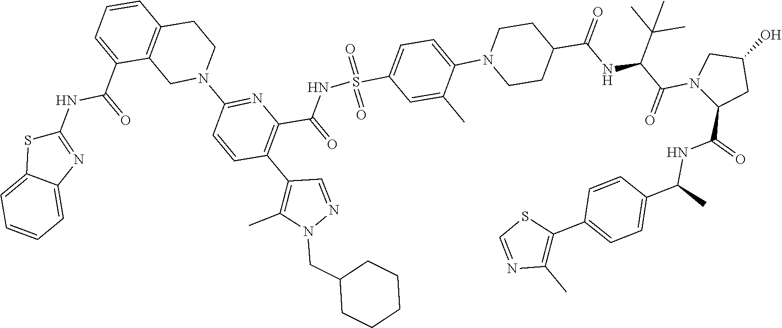 [SMILES: Cc1cc(S(=O)(=O)NC(=O)c2nc(N3CCc4cccc(C(=O)Nc5nc6ccccc6s5)c4C3)ccc2-c2cnn(CC3CCCCC3)c2C)ccc1N1CCC(C(=O)N[C@H](C(=O)N2C[C@H](O)C[C@H]2C(=O)N[C@@H](C)c2ccc(-c3scnc3C)cc2)C(C)(C)C)CC1] |
| I-274 | 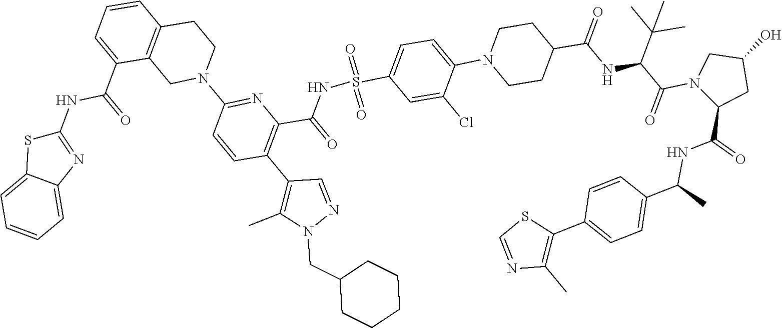 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3Cl)CC2)C(C)(C)C)cc1] |
| I-275 | 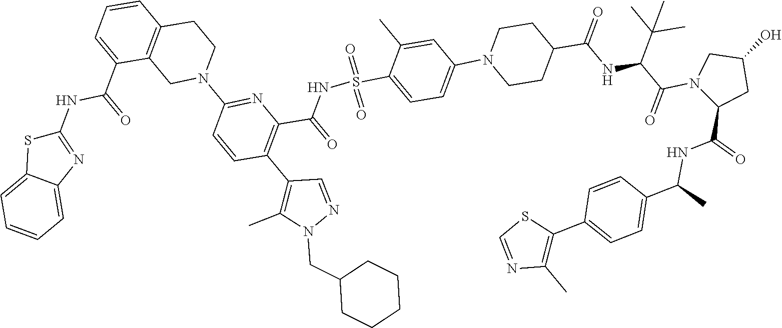 [SMILES: Cc1cc(N2CCC(C(=O)N[C@H](C(=O)N3C[C@H](O)C[C@H]3C(=O)N[C@@H](C)c3ccc(-c4scnc4C)cc3)C(C)(C)C)CC2)ccc1S(=O)(=O)NC(=O)c1nc(N2CCc3cccc(C(=O)Nc4nc5ccccc5s4)c3C2)ccc1-c1cnn(CC2CCCCC2)c1C] |
| I-276 | 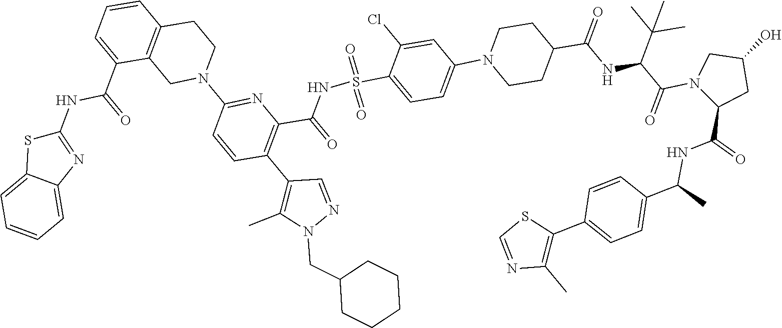 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)c(Cl)c3)CC2)C(C)(C)C)cc1] |
| I-277 | 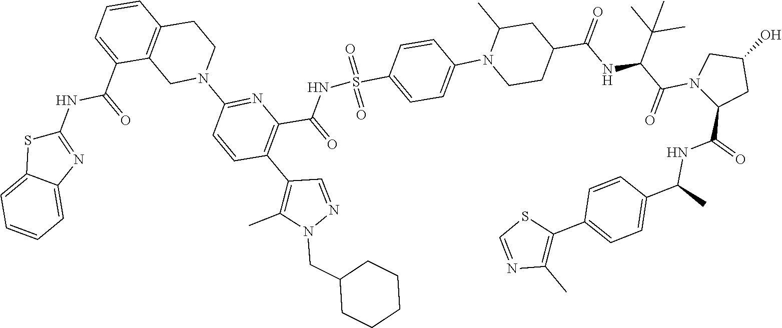 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C(C)C2)C(C)(C)C)cc1] |
| I-278 | 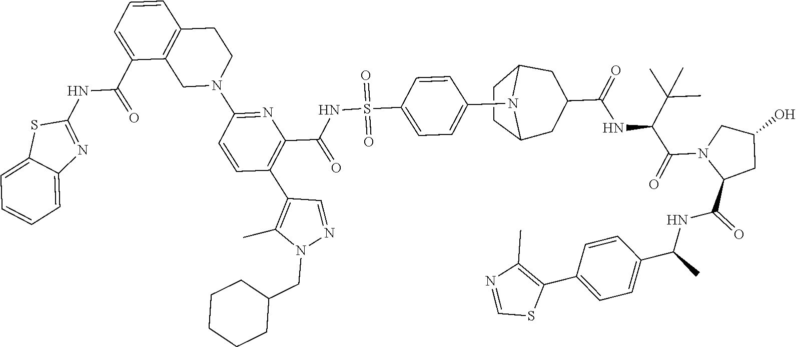 [SMILES: Cc1ncsc1-c1ccc([C@H](C)NC(=O)[C@@H]2C[C@@H](O)CN2C(=O)[C@@H](NC(=O)C2CC3CCC(C2)N3c2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-279 | 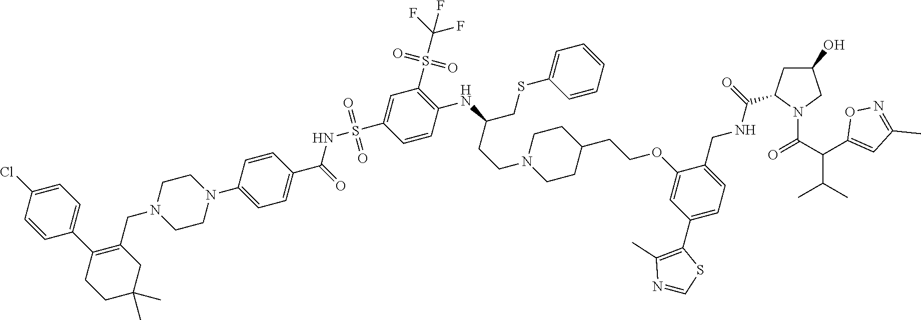 [SMILES: Cc1cc(C(C(=O)N2C[C@H](O)C[C@H]2C(=O)NCc2ccc(-c3scnc3C)cc2OCCC2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-280 | 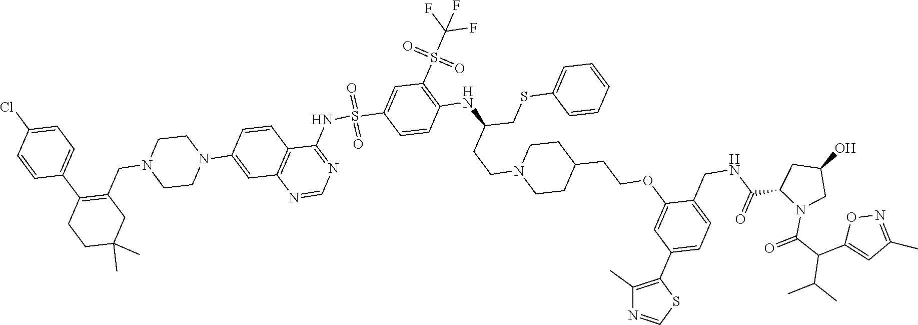 [SMILES: Cc1cc(C(C(=O)N2C[C@H](O)C[C@H]2C(=O)NCc2ccc(-c3scnc3C)cc2OCCC2CCN(CC[C@H](CSc3ccccc3)Nc3ccc(S(=O)(=O)Nc4ncnc5cc(N6CCN(CC7=C(c8ccc(Cl)cc8)CCC(C)(C)C7)CC6)ccc45)cc3S(=O)(=O)C(F)(F)F)CC2)C(C)C)on1] |
| I-281 | 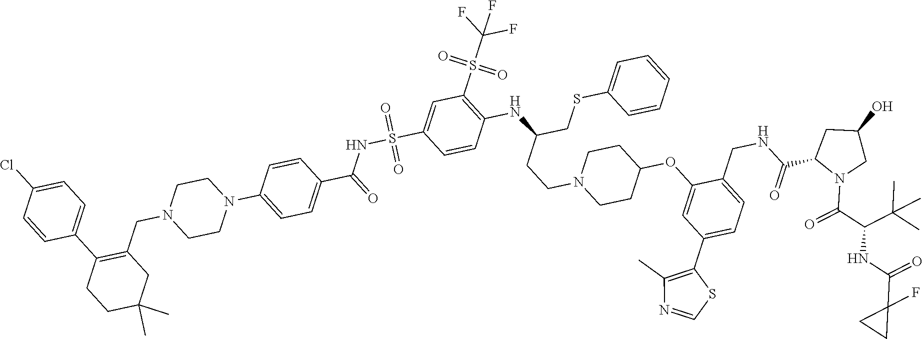 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-282 | 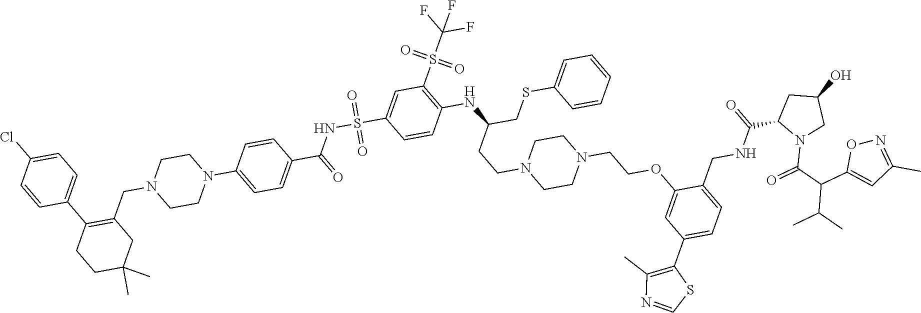 [SMILES: Cc1cc(C(C(=O)N2C[C@H](O)C[C@H]2C(=O)NCc2ccc(-c3scnc3C)cc2OCCN2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-283 | 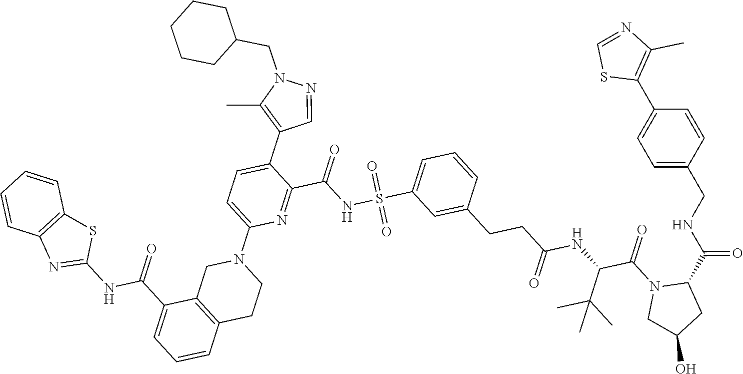 [SMILES: Cc1ncsc1-c1ccc(CNC(=O)[C@@H]2C[C@@H](O)CN2C(=O)[C@@H](NC(=O)CCc2cccc(S(=O)(=O)NC(=O)c3nc(N4CCc5cccc(C(=O)Nc6nc7ccccc7s6)c5C4)ccc3-c3cnn(CC4CCCCC4)c3C)c2)C(C)(C)C)cc1] |
| I-284 | 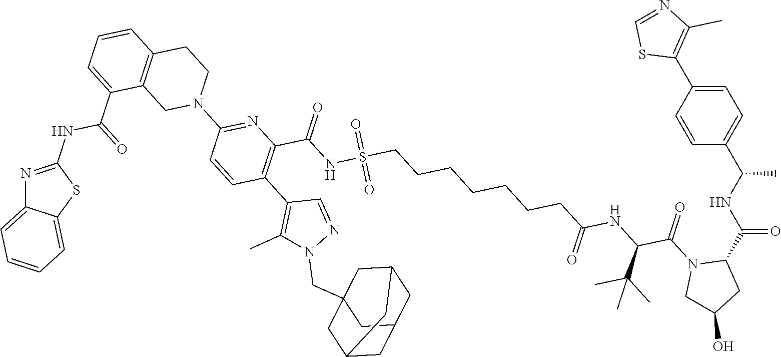 [SMILES: Cc1ncsc1-c1ccc([C@H](C)NC(=O)[C@@H]2C[C@@H](O)CN2C(=O)[C@H](NC(=O)CCCCCCCS(=O)(=O)NC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| I-285 | 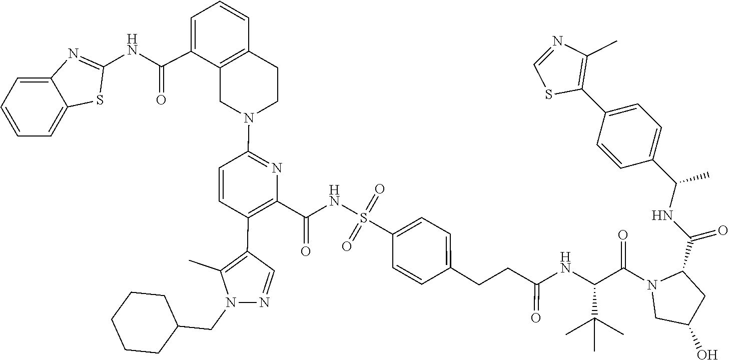 [SMILES: Cc1ncsc1-c1ccc([C@H](C)NC(=O)[C@@H]2C[C@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-286 | 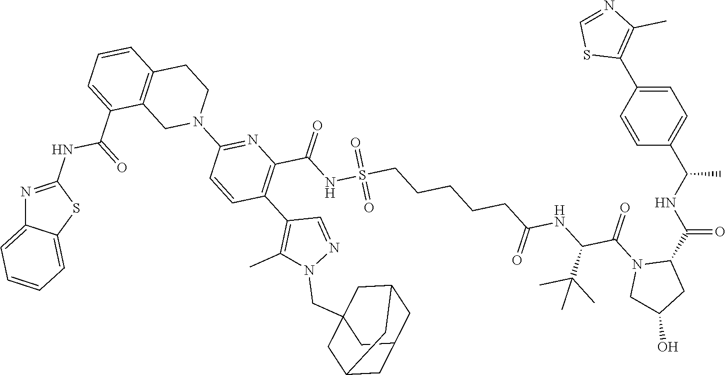 [SMILES: Cc1ncsc1-c1ccc([C@H](C)NC(=O)[C@@H]2C[C@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC34CC5CC(CC(C5)C3)C4)c2C)C(C)(C)C)cc1] |
| I-287 | 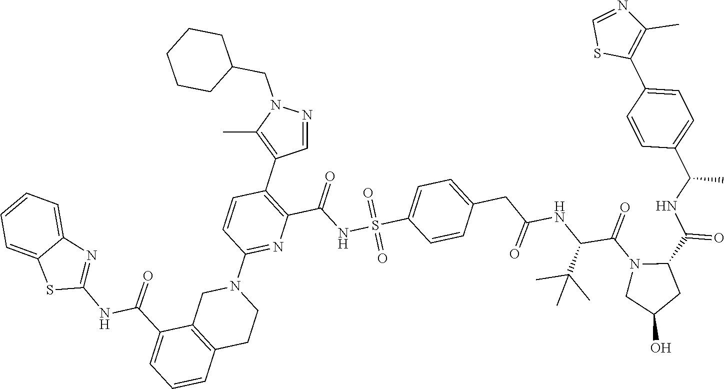 [SMILES: Cc1ncsc1-c1ccc([C@H](C)NC(=O)[C@@H]2C[C@@H](O)CN2C(=O)[C@@H](NC(=O)Cc2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-288 | 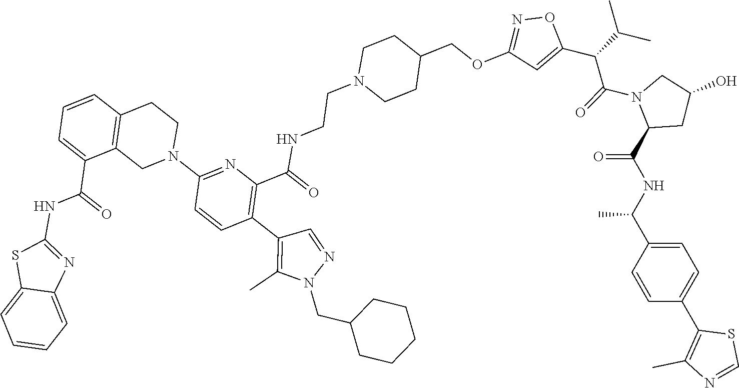 [SMILES: Cc1ncsc1-c1ccc([C@H](C)NC(=O)[C@@H]2C[C@@H](O)CN2C(=O)[C@H](c2cc(OCC3CCN(CCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-289 | 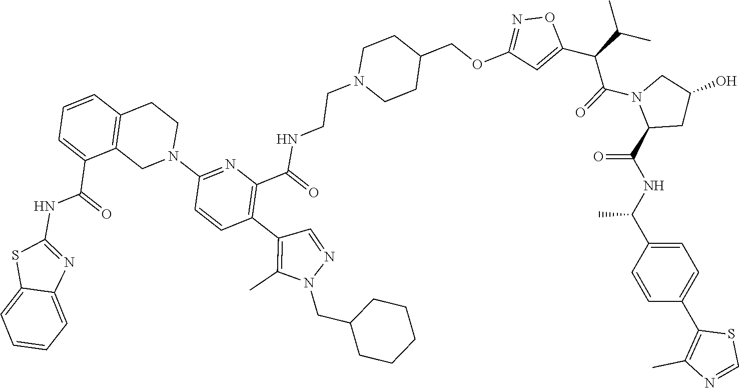 [SMILES: Cc1ncsc1-c1ccc([C@H](C)NC(=O)[C@@H]2C[C@@H](O)CN2C(=O)[C@@H](c2cc(OCC3CCN(CCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-290 | 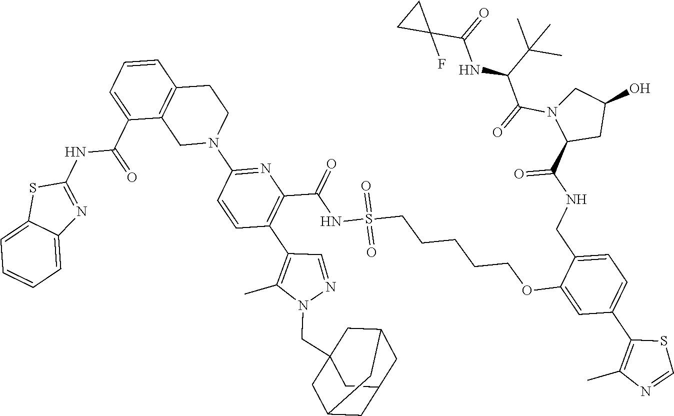 [SMILES: Cc1ncsc1-c1ccc(CNC(=O)[C@@H]2C[C@H](O)CN2C(=O)[C@@H](NC(=O)C2(F)CC2)C(C)(C)C)c(OCCCCCS(=O)(=O)NC(=O)c2nc(N3CCc4cccc(C(=O)Nc5nc6ccccc6s5)c4C3)ccc2-c2cnn(CC34CC5CC(CC(C5)C3)C4)c2C)c1] |
| I-291 | 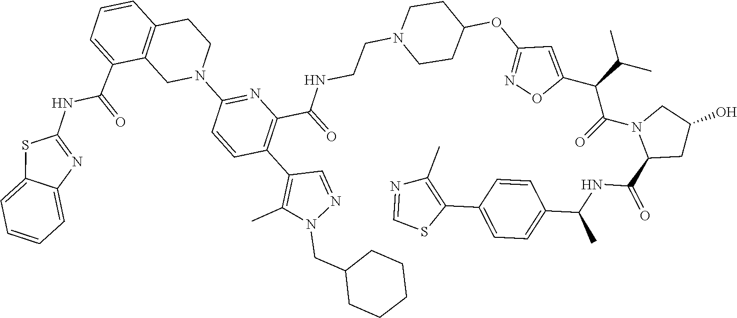 [SMILES: Cc1ncsc1-c1ccc([C@H](C)NC(=O)[C@@H]2C[C@@H](O)CN2C(=O)[C@@H](c2cc(OC3CCN(CCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-292 | 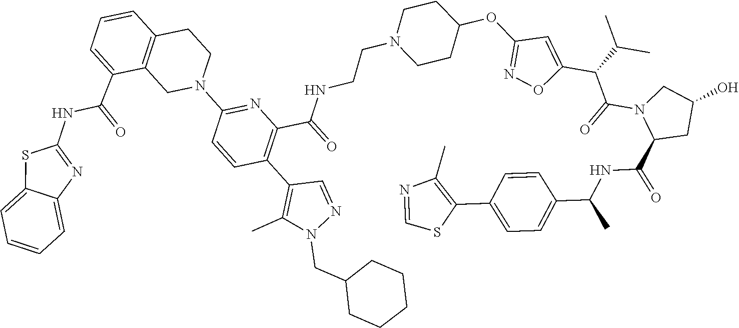 [SMILES: Cc1ncsc1-c1ccc([C@H](C)NC(=O)[C@@H]2C[C@@H](O)CN2C(=O)[C@H](c2cc(OC3CCN(CCNC(=O)c4nc(N5CCc6cccc(C(=O)Nc7nc8ccccc8s7)c6C5)ccc4-c4cnn(CC5CCCCC5)c4C)CC3)no2)C(C)C)cc1] |
| I-293 | 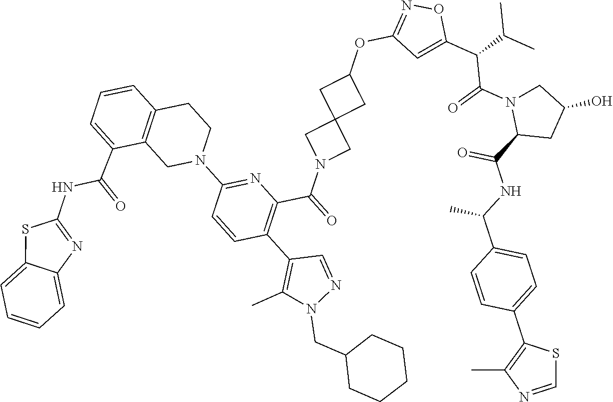 [SMILES: Cc1ncsc1-c1ccc([C@H](C)NC(=O)[C@@H]2C[C@@H](O)CN2C(=O)[C@H](c2cc(OC3CC4(C3)CN(C(=O)c3nc(N5CCc6cccc(C(=O)Nc7nc8ccccc8s7)c6C5)ccc3-c3cnn(CC5CCCCC5)c3C)C4)no2)C(C)C)cc1] |
| I-294 | 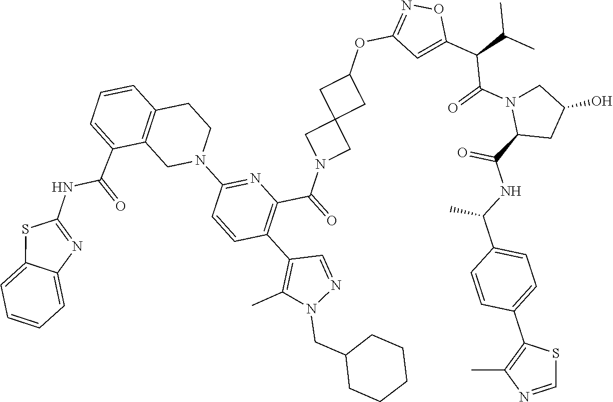 [SMILES: Cc1ncsc1-c1ccc([C@H](C)NC(=O)[C@@H]2C[C@@H](O)CN2C(=O)[C@@H](c2cc(OC3CC4(C3)CN(C(=O)c3nc(N5CCc6cccc(C(=O)Nc7nc8ccccc8s7)c6C5)ccc3-c3cnn(CC5CCCCC5)c3C)C4)no2)C(C)C)cc1] |
| I-295 | 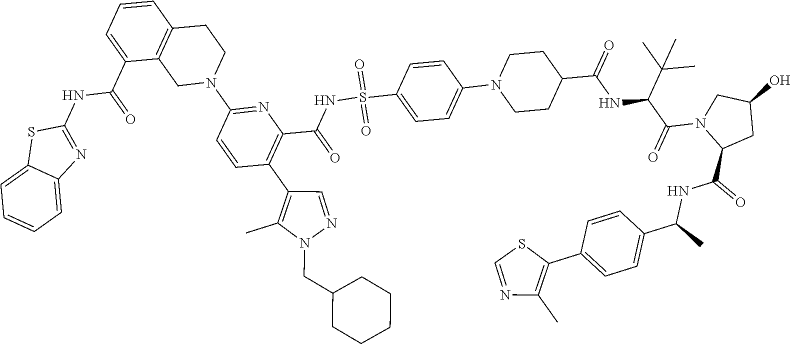 [SMILES: Cc1ncsc1-c1ccc([C@H](C)NC(=O)[C@@H]2C[C@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-296 | 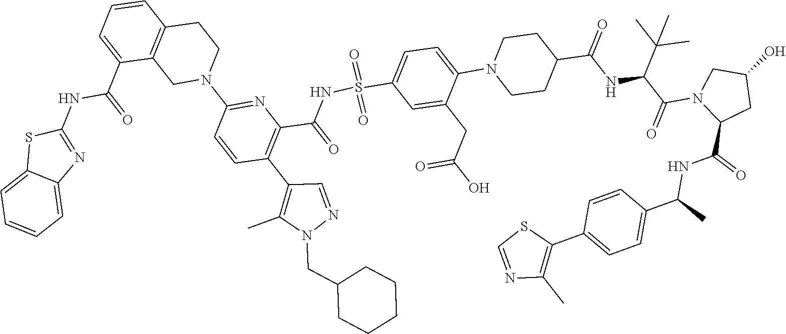 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3CC(=O)O)CC2)C(C)(C)C)cc1] |
| I-297 | 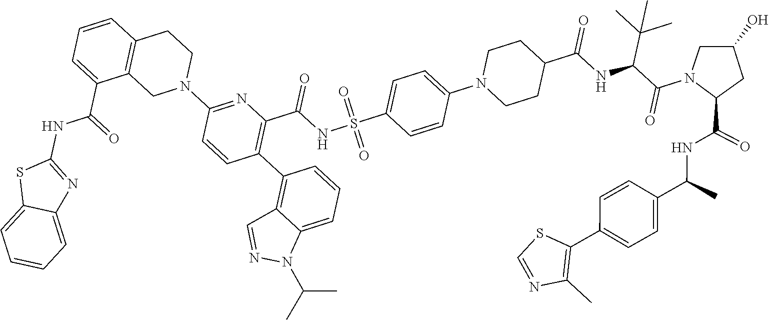 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cccc5c4cnn5C(C)C)cc3)CC2)C(C)(C)C)cc1] |
| I-298 | 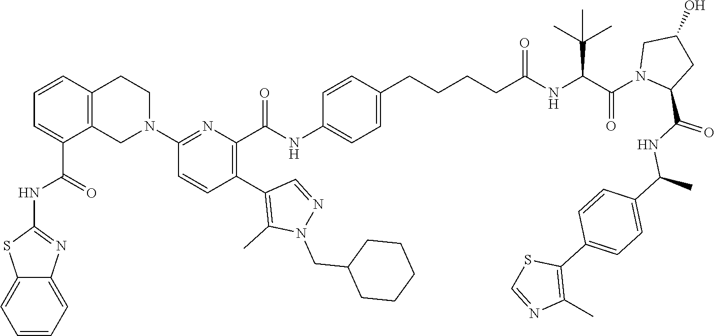 [SMILES: Cc1ncsc1-c1ccc([C@H](C)NC(=O)[C@@H]2C[C@@H](O)CN2C(=O)[C@@H](NC(=O)CCCCc2ccc(NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-299 | 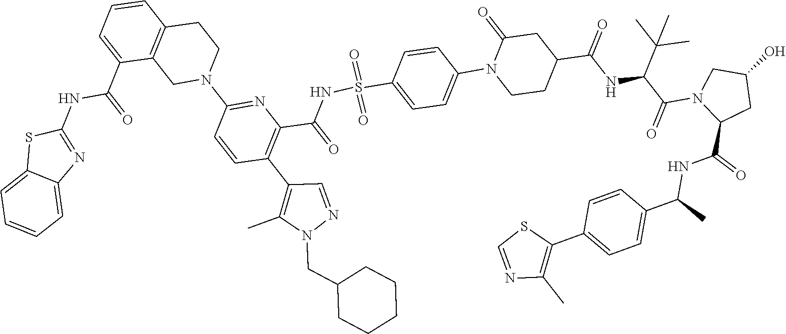 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C(=O)C2)C(C)(C)C)cc1] |
| I-300 | 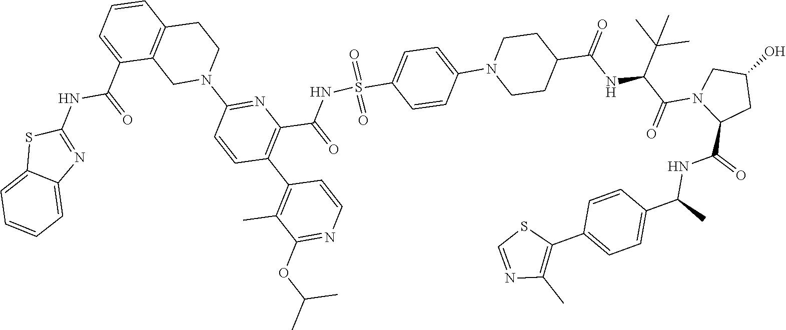 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4ccnc(OC(C)C)c4C)cc3)CC2)C(C)(C)C)cc1] |
| I-301 | 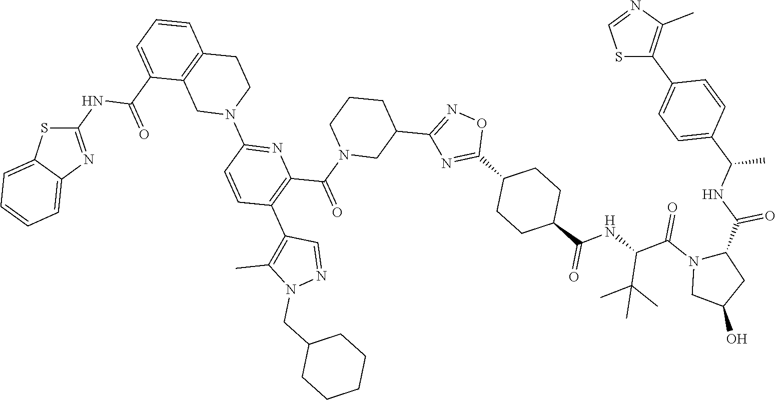 [SMILES: Cc1ncsc1-c1ccc([C@H](C)NC(=O)[C@@H]2C[C@@H](O)CN2C(=O)[C@@H](NC(=O)[C@H]2CC[C@H](c3nc(C4CCCN(C(=O)c5nc(N6CCc7cccc(C(=O)Nc8nc9ccccc9s8)c7C6)ccc5-c5cnn(CC6CCCCC6)c5C)C4)no3)CC2)C(C)(C)C)cc1] |
| I-302 | 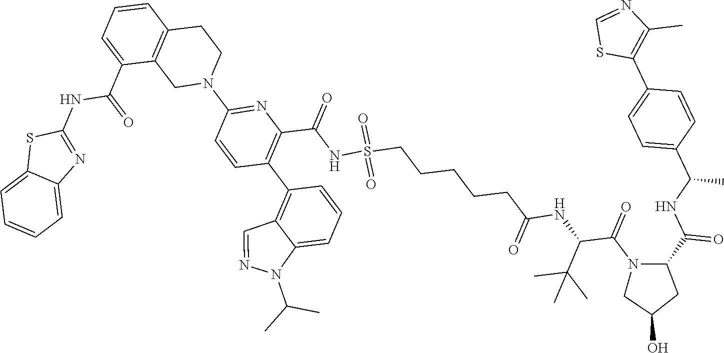 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cccc3c2cnn3C(C)C)C(C)(C)C)cc1] |
| I-303 | 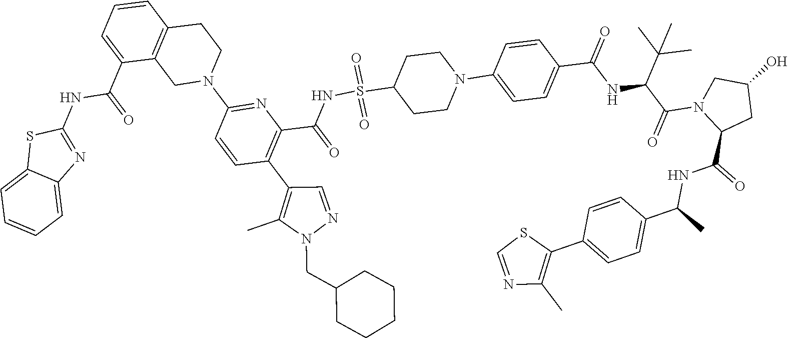 [SMILES: Cc1ncsc1-c1ccc([C@H](C)NC(=O)[C@@H]2C[C@@H](O)CN2C(=O)[C@@H](NC(=O)c2ccc(N3CCC(S(=O)(=O)NC(=O)c4nc(N5CCc6cccc(C(=O)Nc7nc8ccccc8s7)c6C5)ccc4-c4cnn(CC5CCCCC5)c4C)CC3)cc2)C(C)(C)C)cc1] |
| I-304 | 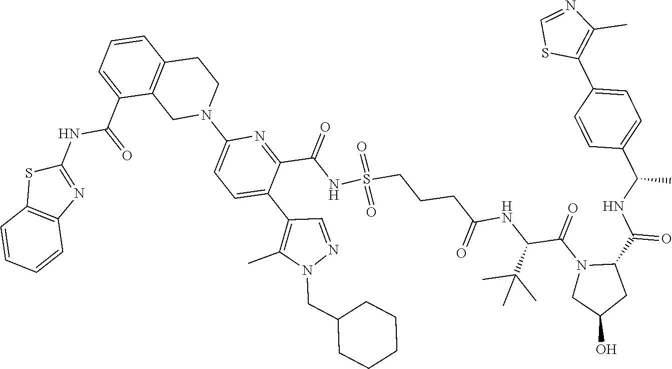 [SMILES: Cc1ncsc1-c1ccc([C@H](C)NC(=O)[C@@H]2C[C@@H](O)CN2C(=O)[C@@H](NC(=O)CCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-305 | 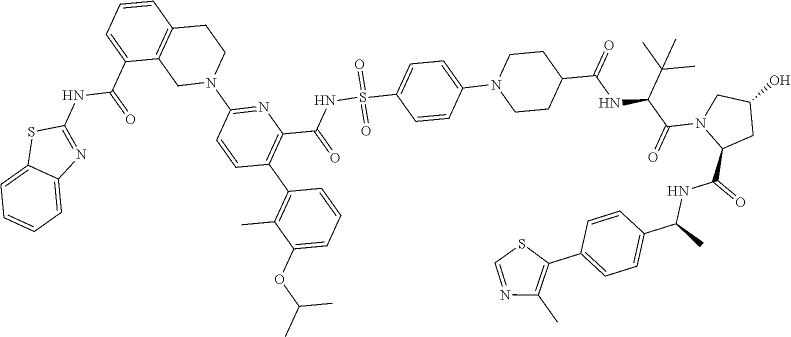 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cccc(OC(C)C)c4C)cc3)CC2)C(C)(C)C)cc1] |
| I-306 | 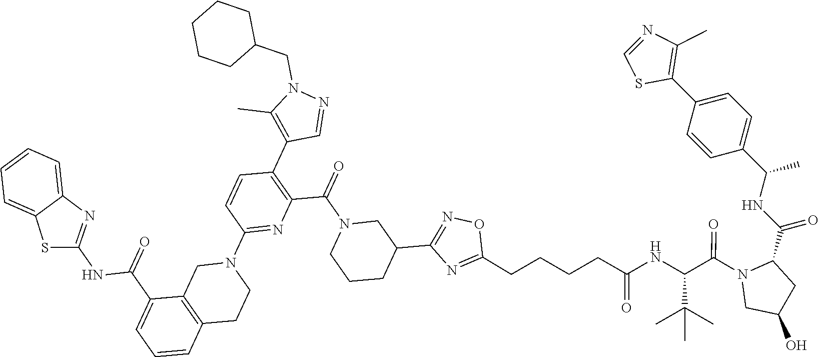 [SMILES: Cc1ncsc1-c1ccc([C@H](C)NC(=O)[C@@H]2C[C@@H](O)CN2C(=O)[C@@H](NC(=O)CCCCc2nc(C3CCCN(C(=O)c4nc(N5CCc6cccc(C(=O)Nc7nc8ccccc8s7)c6C5)ccc4-c4cnn(CC5CCCCC5)c4C)C3)no2)C(C)(C)C)cc1] |
| I-307 | 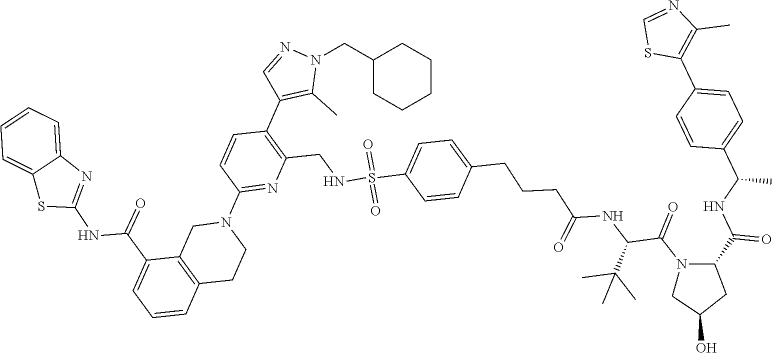 [SMILES: Cc1ncsc1-c1ccc([C@H](C)NC(=O)[C@@H]2C[C@@H](O)CN2C(=O)[C@@H](NC(=O)CCCc2ccc(S(=O)(=O)NCc3nc(N4CCc5cccc(C(=O)Nc6nc7ccccc7s6)c5C4)ccc3-c3cnn(CC4CCCCC4)c3C)cc2)C(C)(C)C)cc1] |
| I-308 | 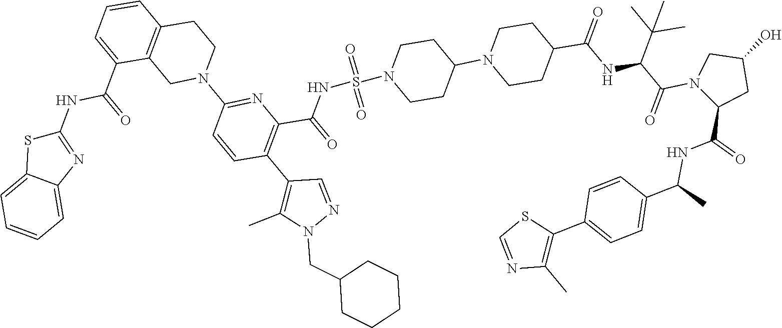 [SMILES: Cc1ncsc1-c1ccc([C@H](C)NC(=O)[C@@H]2C[C@@H](O)CN2C(=O)[C@@H](NC(=O)C2CCN(C3CCN(S(=O)(=O)NC(=O)c4nc(N5CCc6cccc(C(=O)Nc7nc8ccccc8s7)c6C5)ccc4-c4cnn(CC5CCCCC5)c4C)CC3)CC2)C(C)(C)C)cc1] |
| I-309 | 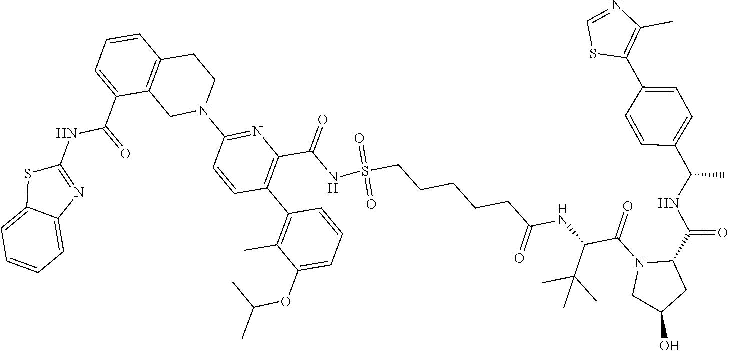 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cccc(OC(C)C)c2C)C(C)(C)C)cc1] |
| I-310 | 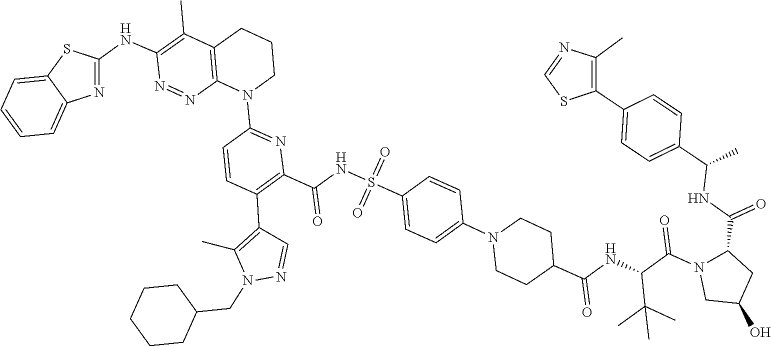 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCCc6c5nnc(Nc5nc7ccccc7s5)c6C)ccc4-c4cnn(CC5CCCCC5)c4C)cc3)CC2)C(C)(C)C)cc1] |
| I-311 | 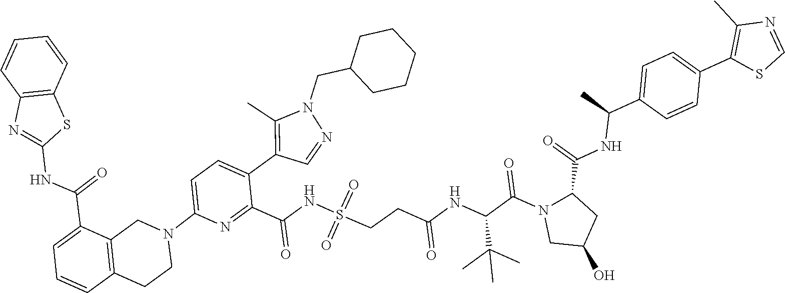 [SMILES: Cc1ncsc1-c1ccc([C@H](C)NC(=O)[C@@H]2C[C@@H](O)CN2C(=O)[C@@H](NC(=O)CCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-312 | 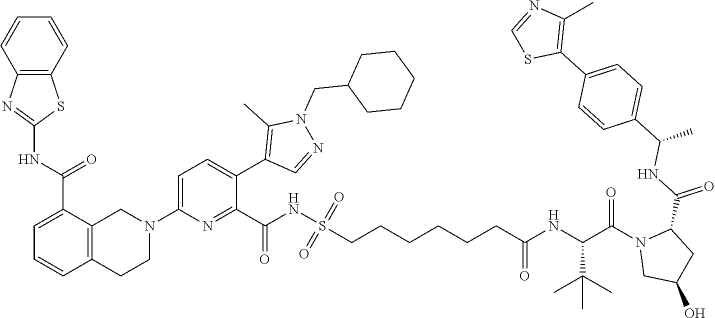 [SMILES: Cc1ncsc1-c1ccc([C@H](C)NC(=O)[C@@H]2C[C@@H](O)CN2C(=O)[C@@H](NC(=O)CCCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-313 | 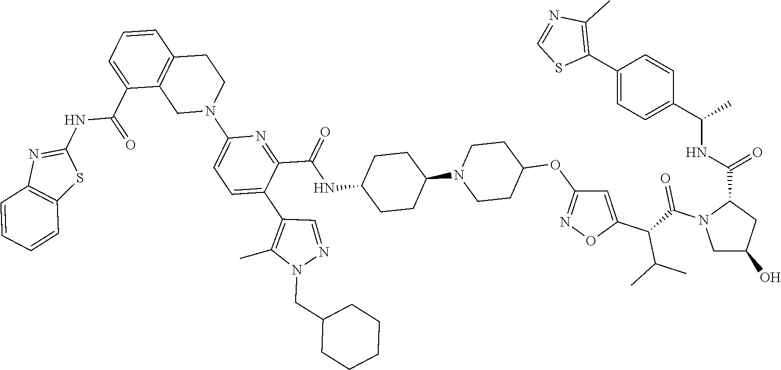 [SMILES: Cc1ncsc1-c1ccc([C@H](C)NC(=O)[C@@H]2C[C@@H](O)CN2C(=O)[C@@H](c2cc(OC3CCN([C@H]4CC[C@H](NC(=O)c5nc(N6CCc7cccc(C(=O)Nc8nc9ccccc9s8)c7C6)ccc5-c5cnn(CC6CCCCC6)c5C)CC4)CC3)no2)C(C)C)cc1] |
| I-314 | 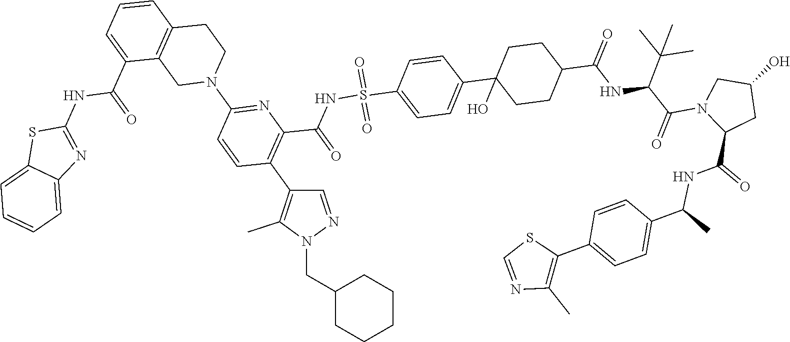 [SMILES: Cc1ncsc1-c1ccc([C@H](C)NC(=O)[C@@H]2C[C@@H](O)CN2C(=O)[C@@H](NC(=O)C2CCC(O)(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-315 | 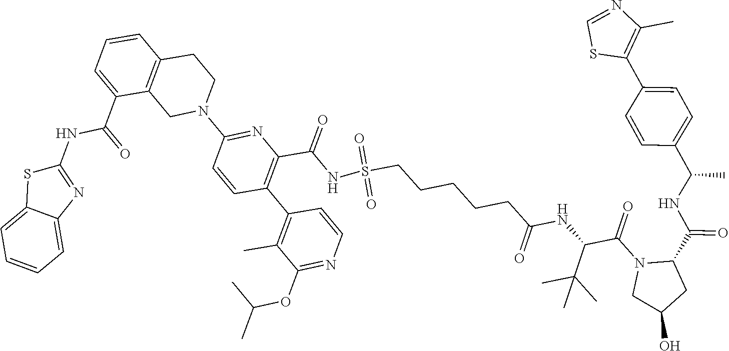 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2ccnc(OC(C)C)c2C)C(C)(C)C)cc1] |
| I-316 | 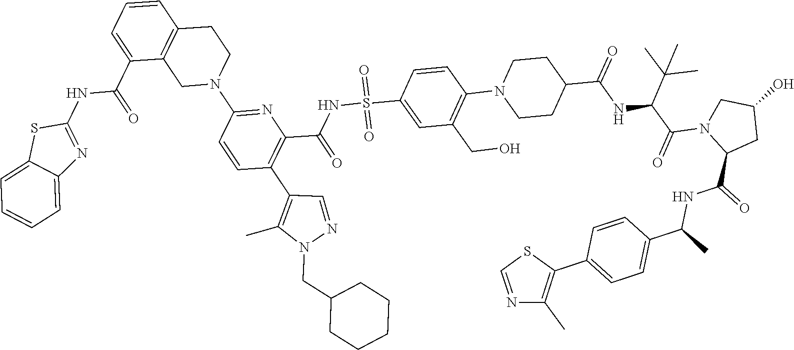 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3CO)CC2)C(C)(C)C)cc1] |
| I-317 | 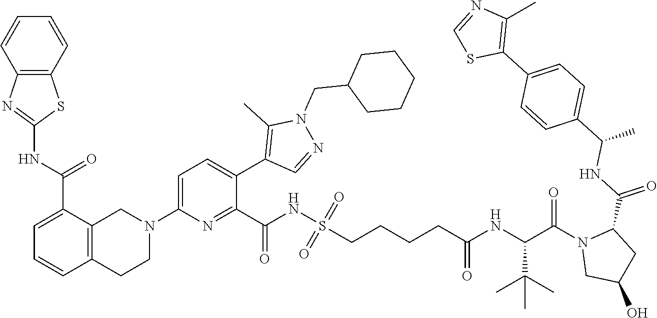 [SMILES: Cc1ncsc1-c1ccc([C@H](C)NC(=O)[C@@H]2C[C@@H](O)CN2C(=O)[C@@H](NC(=O)CCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-318 | 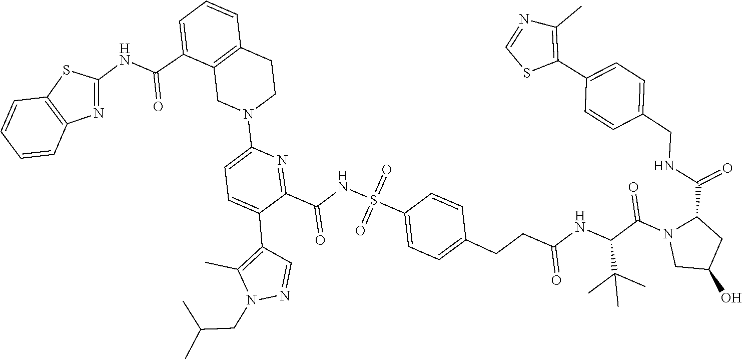 [SMILES: Cc1ncsc1-c1ccc(CNC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC(C)C)c3C)cc2)C(C)(C)C)cc1] |
| I-319 | 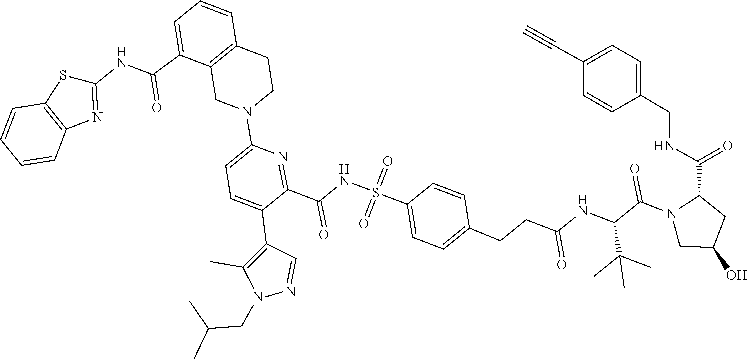 [SMILES: C#Cc1ccc(CNC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC(C)C)c3C)cc2)C(C)(C)C)cc1] |
| I-320 | 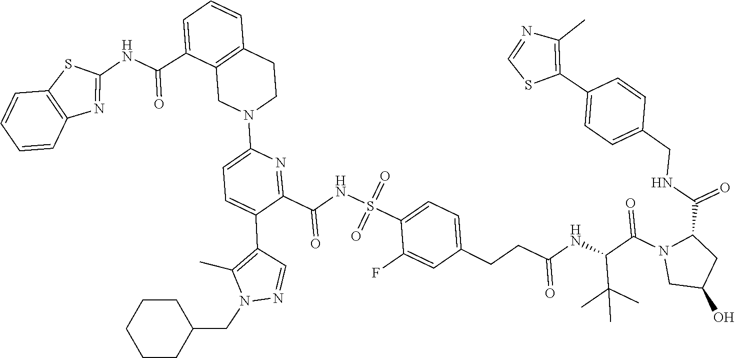 [SMILES: Cc1ncsc1-c1ccc(CNC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC4CCCCC4)c3C)c(F)c2)C(C)(C)C)cc1] |
| I-321 | 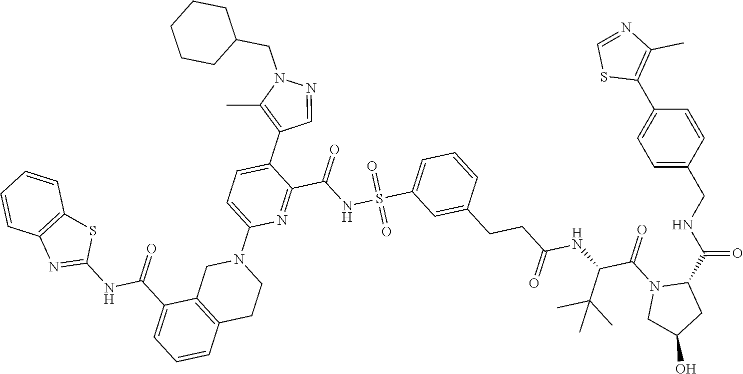 [SMILES: Cc1ncsc1-c1ccc(CNC(=O)[C@@H]2C[C@@H](O)CN2C(=O)[C@@H](NC(=O)CCc2cccc(S(=O)(=O)NC(=O)c3nc(N4CCc5cccc(C(=O)Nc6nc7ccccc7s6)c5C4)ccc3-c3cnn(CC4CCCCC4)c3C)c2)C(C)(C)C)cc1] |
| I-322 | 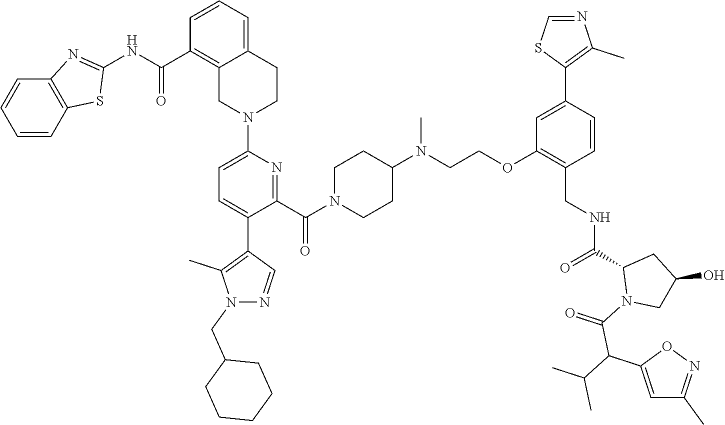 [SMILES: Cc1cc(C(C(=O)N2C[C@H](O)C[C@H]2C(=O)NCc2ccc(-c3scnc3C)cc2OCCN(C)C2CCN(C(=O)c3nc(N4CCc5cccc(C(=O)Nc6nc7ccccc7s6)c5C4)ccc3-c3cnn(CC4CCCCC4)c3C)CC2)C(C)C)on1] |
| I-323 | 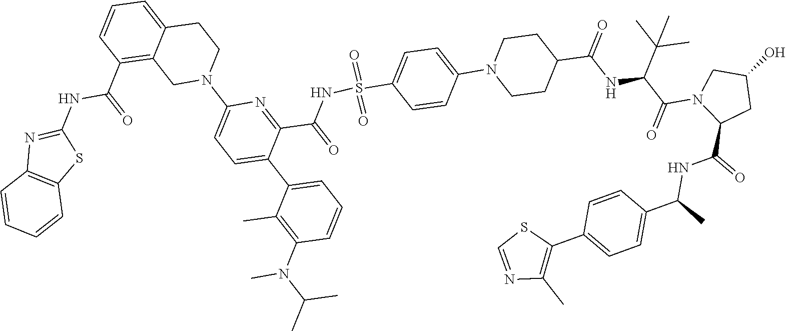 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cccc(N(C)C(C)C)c4C)cc3)CC2)C(C)(C)C)cc1] |
| I-324 | 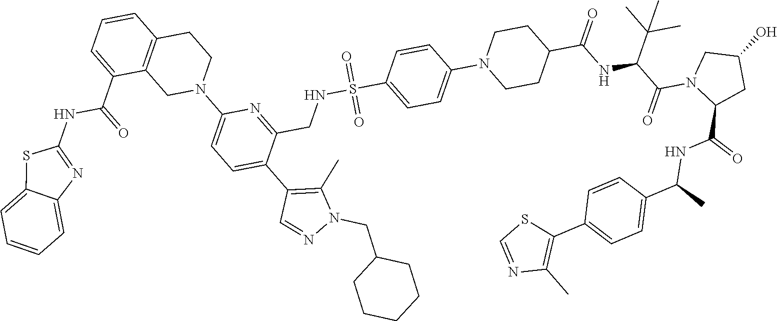 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NCc4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-325 | 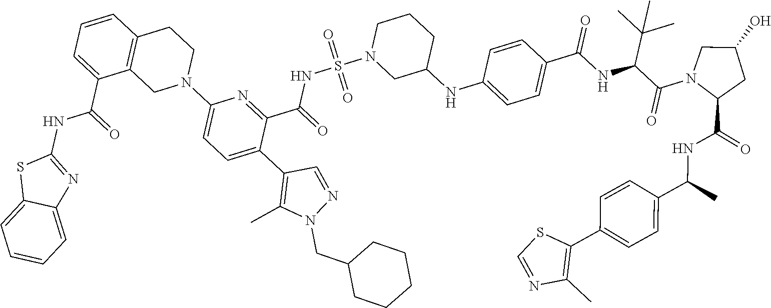 [SMILES: Cc1ncsc1-c1ccc([C@H](C)NC(=O)[C@@H]2C[C@@H](O)CN2C(=O)[C@@H](NC(=O)c2ccc(NC3CCCN(S(=O)(=O)NC(=O)c4nc(N5CCc6cccc(C(=O)Nc7nc8ccccc8s7)c6C5)ccc4-c4cnn(CC5CCCCC5)c4C)C3)cc2)C(C)(C)C)cc1] |
| I-326 | 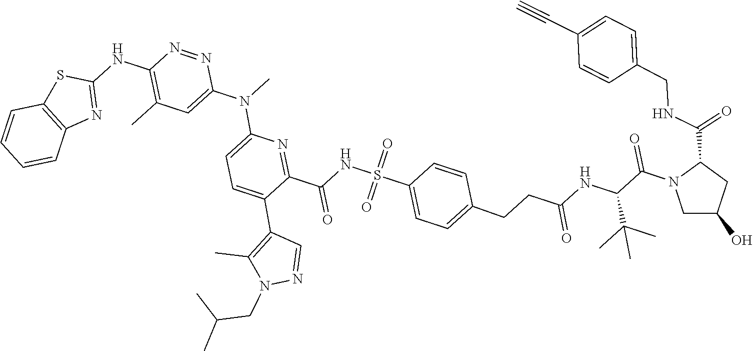 [SMILES: C#Cc1ccc(CNC(=O)[C@@H]2C[C@@H](O)CN2C(=O)[C@@H](NC(=O)CCc2ccc(S(=O)(=O)NC(=O)c3nc(N(C)c4cc(C)c(Nc5nc6ccccc6s5)nn4)ccc3-c3cnn(CC(C)C)c3C)cc2)C(C)(C)C)cc1] |
| I-327 | 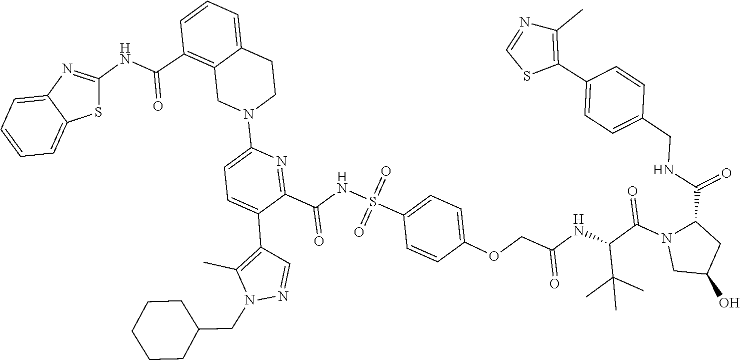 [SMILES: Cc1ncsc1-c1ccc(CNC(=O)[C@@H]2C[C@@H](O)CN2C(=O)[C@@H](NC(=O)COc2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C(C)(C)C)cc1] |
| I-328 | 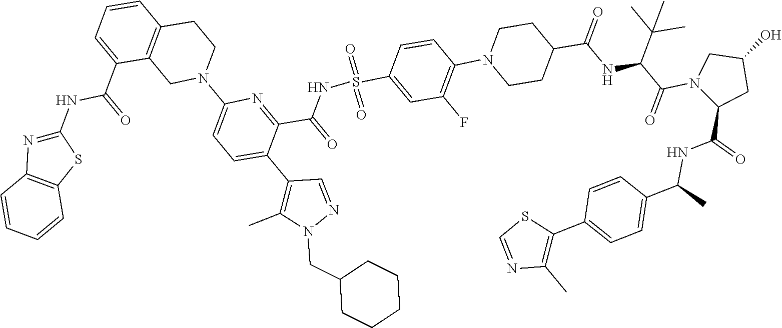 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3F)CC2)C(C)(C)C)cc1] |
| I-329 | 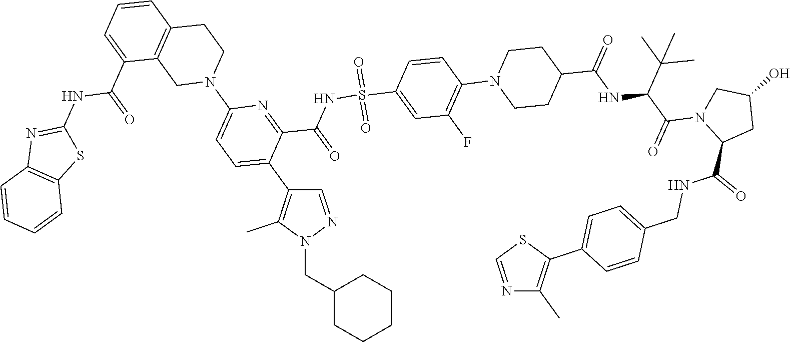 [SMILES: Cc1ncsc1-c1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3F)CC2)C(C)(C)C)cc1] |
| I-330 | 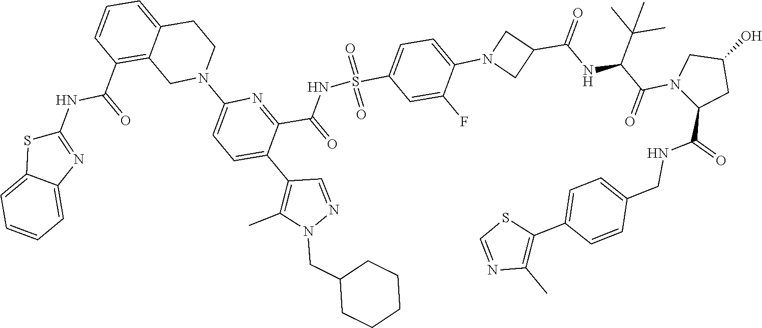 [SMILES: Cc1ncsc1-c1ccc(CNC(=O)[C@@H]2C[C@@H](O)CN2C(=O)[C@@H](NC(=O)C2CN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3F)C2)C(C)(C)C)cc1] |
| I-331 | 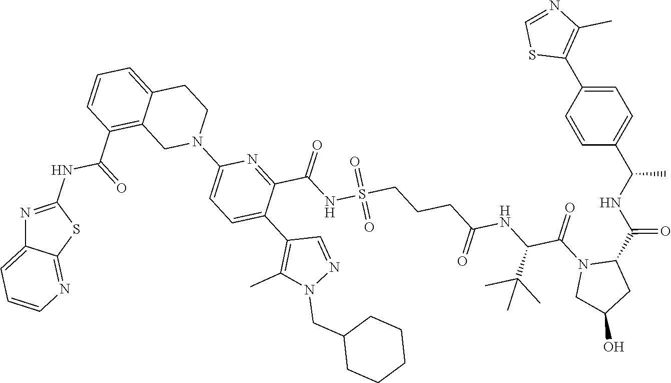 [SMILES: Cc1ncsc1-c1ccc([C@H](C)NC(=O)[C@@H]2C[C@@H](O)CN2C(=O)[C@@H](NC(=O)CCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-332 | 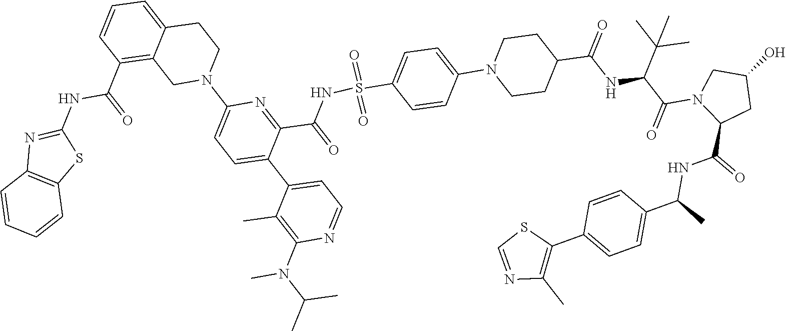 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4ccnc(N(C)C(C)C)c4C)cc3)CC2)C(C)(C)C)cc1] |
| I-333 | 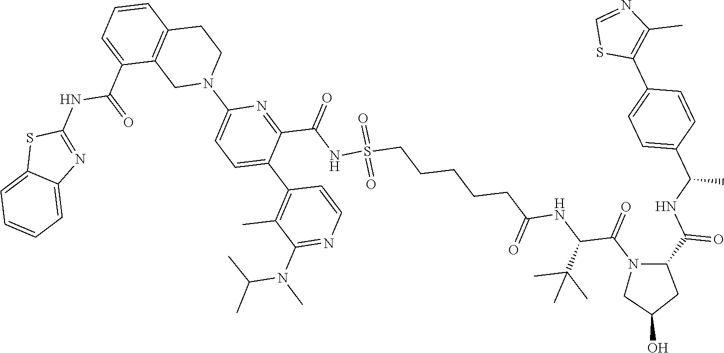 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2ccnc(N(C)C(C)C)c2C)C(C)(C)C)cc1] |
| I-334 | 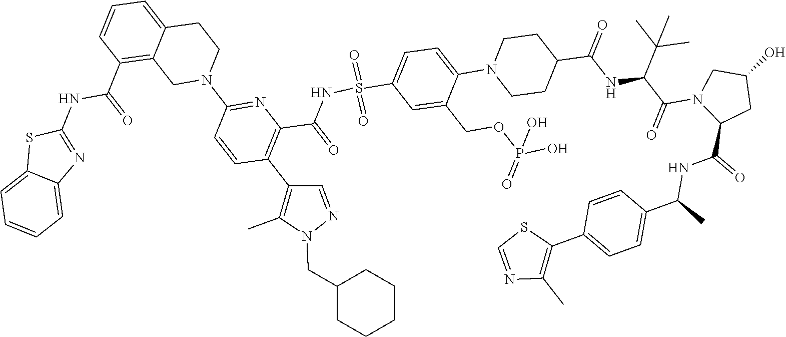 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3COP(=O)(O)O)CC2)C(C)(C)C)cc1] |
| I-335 | 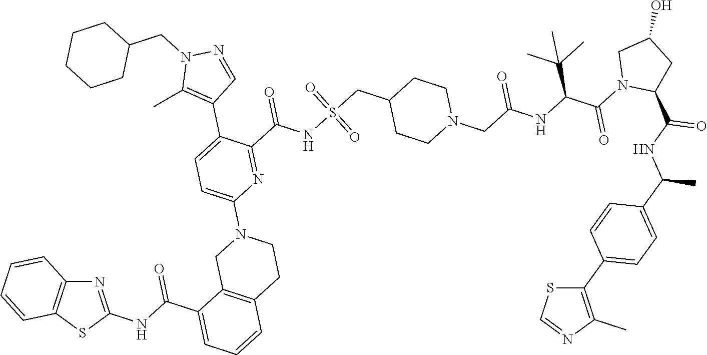 [SMILES: Cc1ncsc1-c1ccc([C@H](C)NC(=O)[C@@H]2C[C@@H](O)CN2C(=O)[C@@H](NC(=O)CN2CCC(CS(=O)(=O)NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-336 | 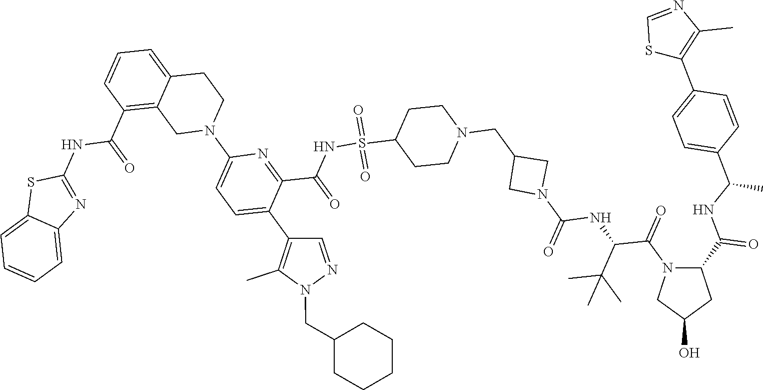 [SMILES: Cc1ncsc1-c1ccc([C@H](C)NC(=O)[C@@H]2C[C@@H](O)CN2C(=O)[C@@H](NC(=O)N2CC(CN3CCC(S(=O)(=O)NC(=O)c4nc(N5CCc6cccc(C(=O)Nc7nc8ccccc8s7)c6C5)ccc4-c4cnn(CC5CCCCC5)c4C)CC3)C2)C(C)(C)C)cc1] |
| I-337 | 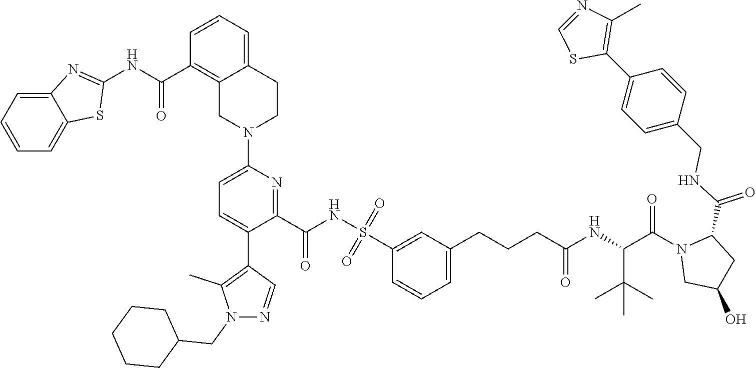 [SMILES: Cc1ncsc1-c1ccc(CNC(=O)[C@@H]2C[C@@H](O)CN2C(=O)[C@@H](NC(=O)CCCc2cccc(S(=O)(=O)NC(=O)c3nc(N4CCc5cccc(C(=O)Nc6nc7ccccc7s6)c5C4)ccc3-c3cnn(CC4CCCCC4)c3C)c2)C(C)(C)C)cc1] |
| I-338 | 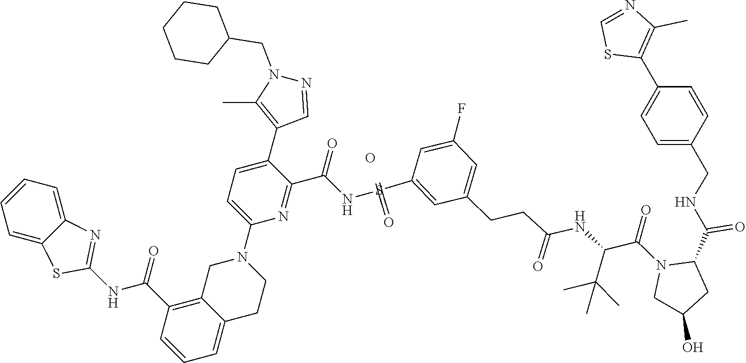 |
| I-339 | 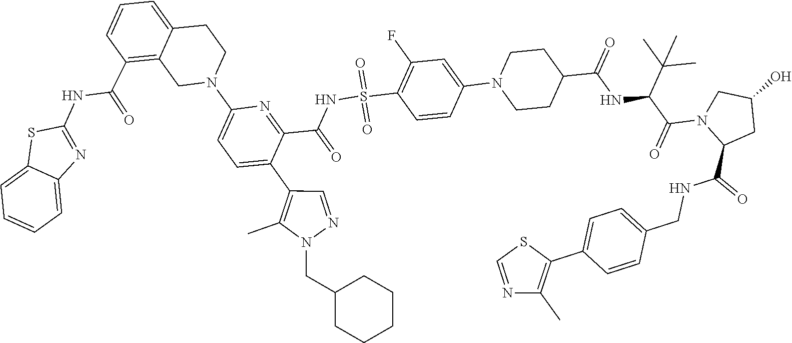 [SMILES: Cc1ncsc1-c1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)c(F)c3)CC2)C(C)(C)C)cc1] |
| I-340 | 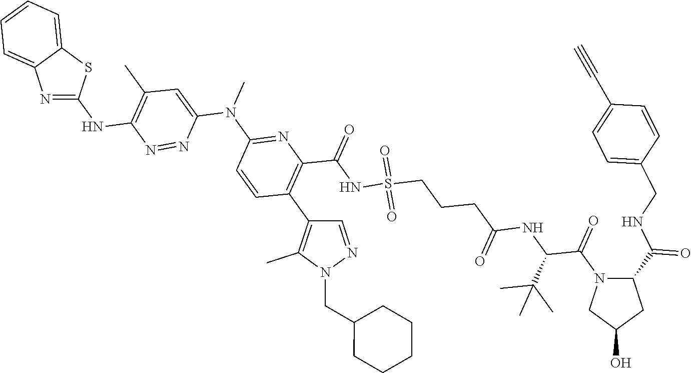 [SMILES: C#Cc1ccc(CNC(=O)[C@@H]2C[C@@H](O)CN2C(=O)[C@@H](NC(=O)CCCS(=O)(=O)NC(=O)c2nc(N(C)c3cc(C)c(Nc4nc5ccccc5s4)nn3)ccc2-c2cnn(CC3CCCCC3)c2C)C(C)(C)C)cc1] |
| I-341 | 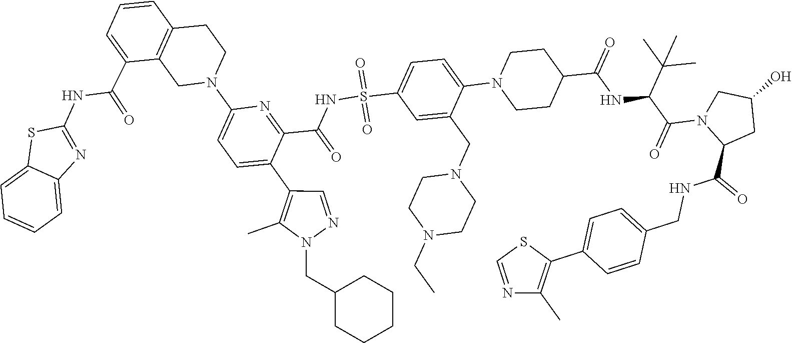 [SMILES: CCN1CCN(Cc2cc(S(=O)(=O)NC(=O)c3nc(N4CCc5cccc(C(=O)Nc6nc7ccccc7s6)c5C4)ccc3-c3cnn(CC4CCCCC4)c3C)ccc2N2CCC(C(=O)N[C@H](C(=O)N3C[C@H](O)C[C@H]3C(=O)NCc3ccc(-c4scnc4C)cc3)C(C)(C)C)CC2)CC1] |
| I-342 | 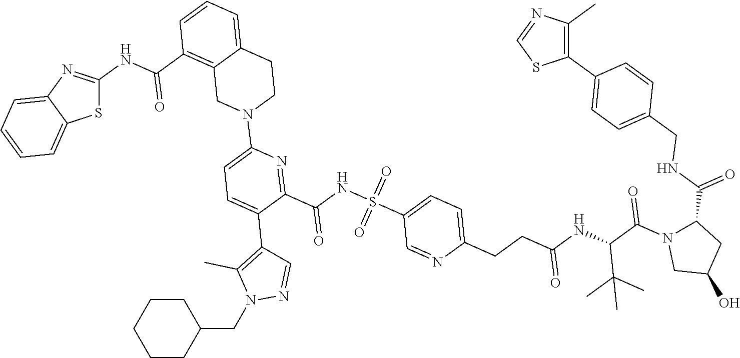 [SMILES: Cc1ncsc1-c1ccc(CNC(=O)[C@@H]2C[C@@H](O)CN2C(=O)[C@@H](NC(=O)CCc2ccc(S(=O)(=O)NC(=O)c3nc(N4CCc5cccc(C(=O)Nc6nc7ccccc7s6)c5C4)ccc3-c3cnn(CC4CCCCC4)c3C)cn2)C(C)(C)C)cc1] |
| I-343 | 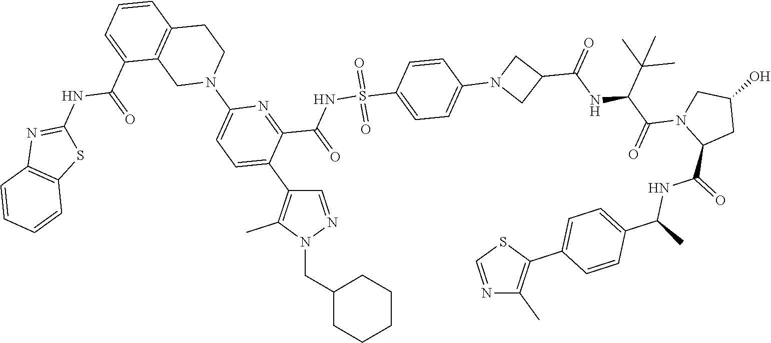 [SMILES: Cc1ncsc1-c1ccc([C@H](C)NC(=O)[C@@H]2C[C@@H](O)CN2C(=O)[C@@H](NC(=O)C2CN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C2)C(C)(C)C)cc1] |
| I-344 | 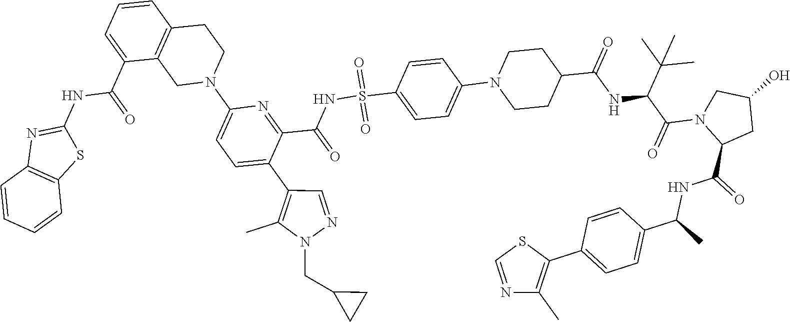 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CC5)c4C)cc3)CC2)C(C)(C)C)cc1] |
| I-345 | 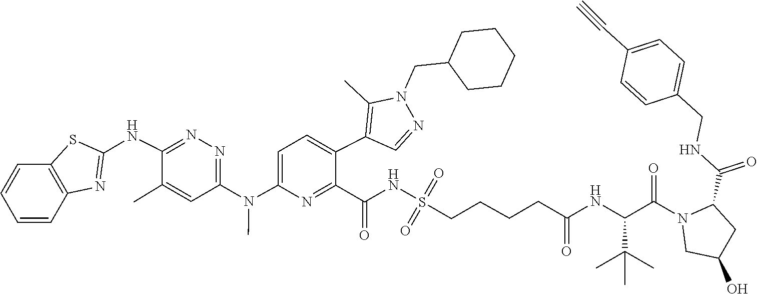 [SMILES: C#Cc1ccc(CNC(=O)[C@@H]2C[C@@H](O)CN2C(=O)[C@@H](NC(=O)CCCCS(=O)(=O)NC(=O)c2nc(N(C)c3cc(C)c(Nc4nc5ccccc5s4)nn3)ccc2-c2cnn(CC3CCCCC3)c2C)C(C)(C)C)cc1] |
| I-346 | 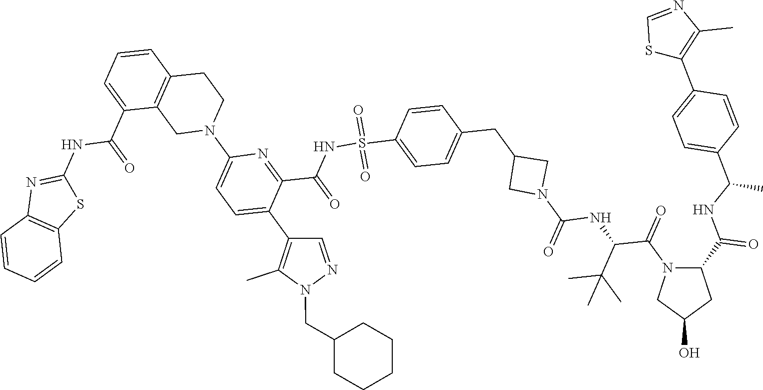 [SMILES: Cc1ncsc1-c1ccc([C@H](C)NC(=O)[C@@H]2C[C@@H](O)CN2C(=O)[C@@H](NC(=O)N2CC(Cc3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C2)C(C)(C)C)cc1] |
| I-347 | 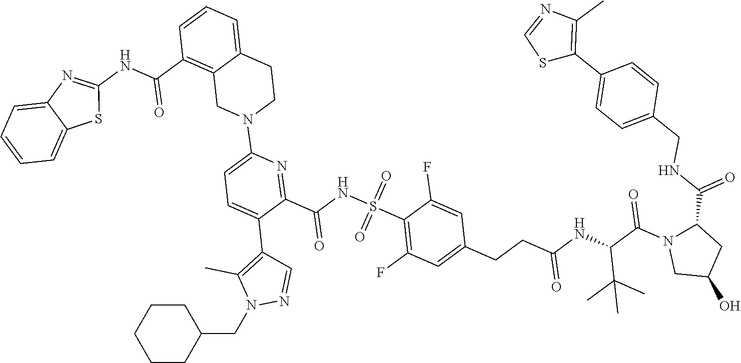 [SMILES: Cc1ncsc1-c1ccc(CNC(=O)[C@@H]2C[C@@H](O)CN2C(=O)[C@@H](NC(=O)CCc2cc(F)c(S(=O)(=O)NC(=O)c3nc(N4CCc5cccc(C(=O)Nc6nc7ccccc7s6)c5C4)ccc3-c3cnn(CC4CCCCC4)c3C)c(F)c2)C(C)(C)C)cc1] |
| I-348 | 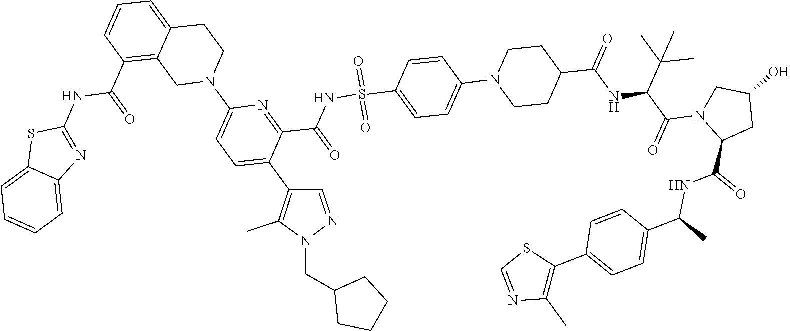 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCC5)c4C)cc3)CC2)C(C)(C)C)cc1] |
| I-349 | 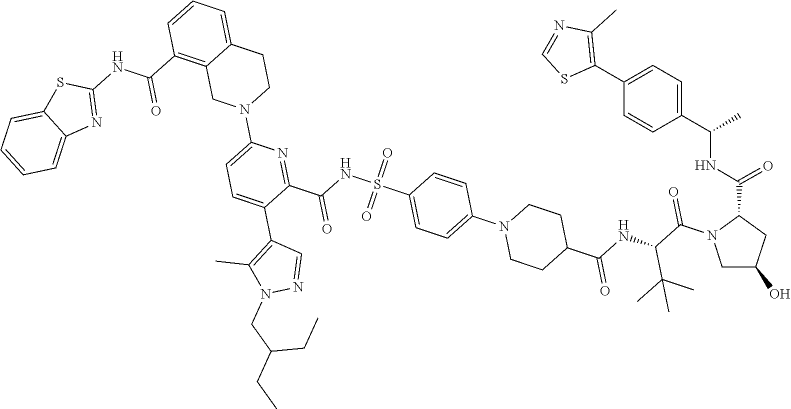 [SMILES: CCC(CC)Cn1ncc(-c2ccc(N3CCc4cccc(C(=O)Nc5nc6ccccc6s5)c4C3)nc2C(=O)NS(=O)(=O)c2ccc(N3CCC(C(=O)N[C@H](C(=O)N4C[C@H](O)C[C@H]4C(=O)N[C@@H](C)c4ccc(-c5scnc5C)cc4)C(C)(C)C)CC3)cc2)c1C] |
| I-350 | 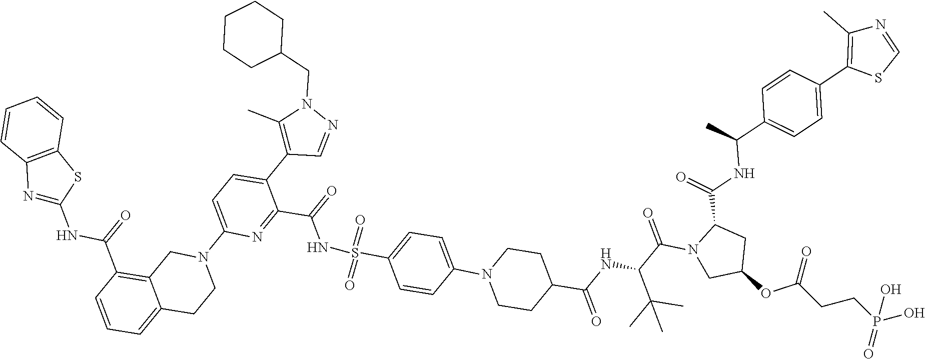 [SMILES: Cc1ncsc1-c1ccc([C@H](C)NC(=O)[C@@H]2C[C@@H](OC(=O)CCP(=O)(O)O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)CC2)C(C)(C)C)cc1] |
| I-351 | 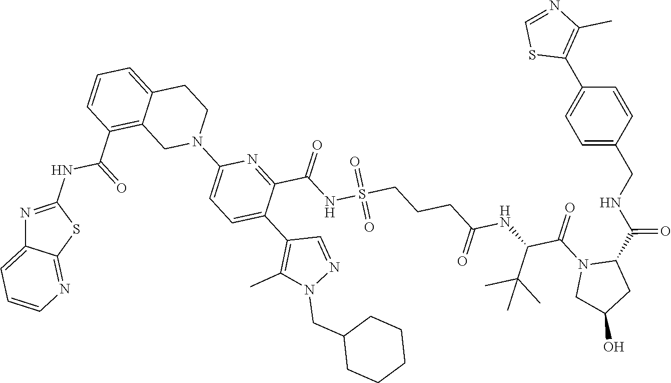 [SMILES: Cc1ncsc1-c1ccc(CNC(=O)[C@@H]2C[C@@H](O)CN2C(=O)[C@@H](NC(=O)CCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-352 | 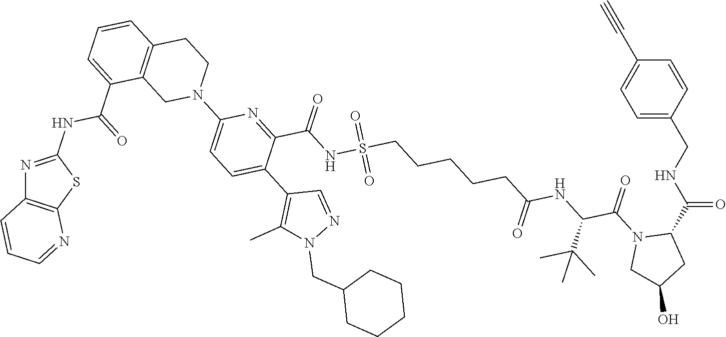 [SMILES: C#Cc1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-353 | 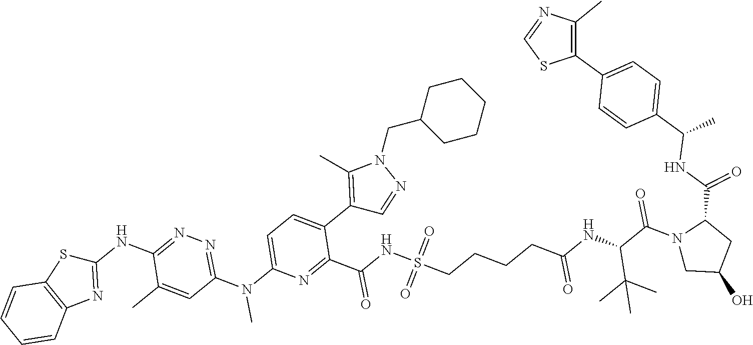 |
| I-354 | 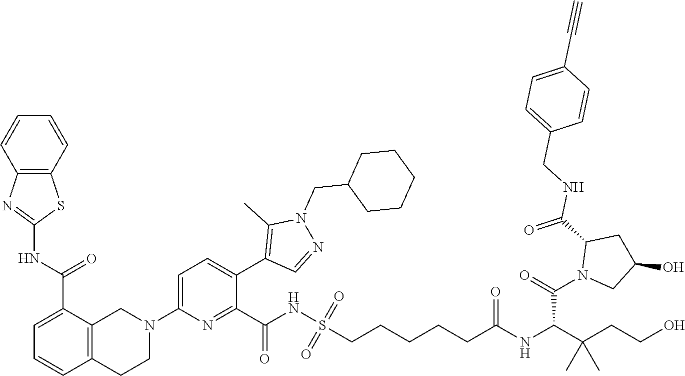 |
| I-355 | 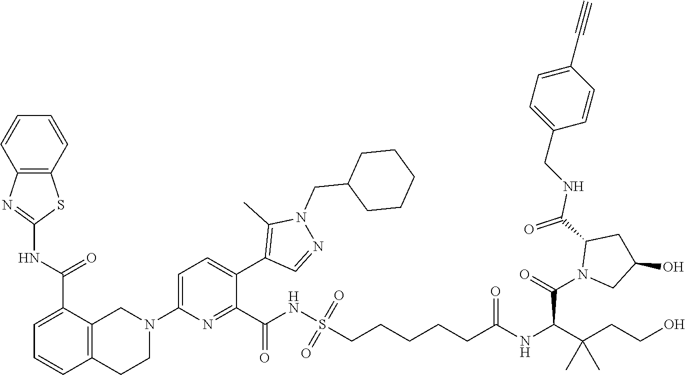 |
| I-356 | 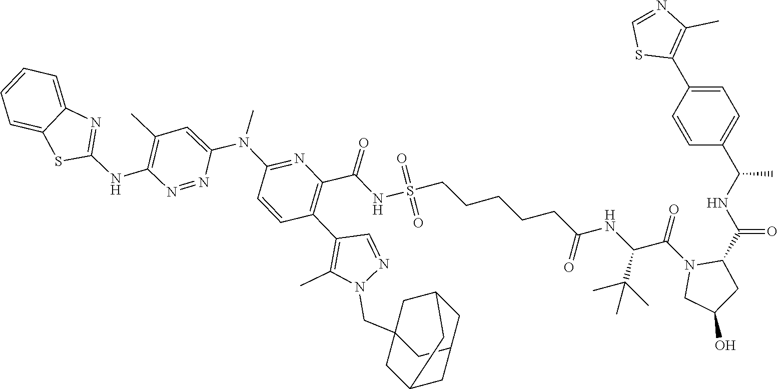 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-357 | 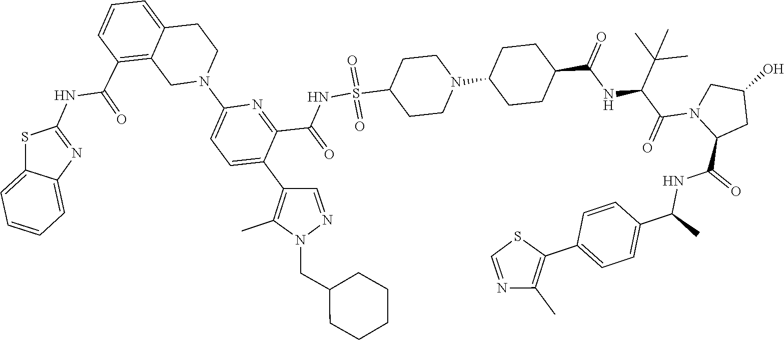 [SMILES: Cc1ncsc1-c1ccc([C@H](C)NC(=O)[C@@H]2C[C@@H](O)CN2C(=O)[C@@H](NC(=O)[C@H]2CC[C@H](N3CCC(S(=O)(=O)NC(=O)c4nc(N5CCc6cccc(C(=O)Nc7nc8ccccc8s7)c6C5)ccc4-c4cnn(CC5CCCCC5)c4C)CC3)CC2)C(C)(C)C)cc1] |
| I-358 | 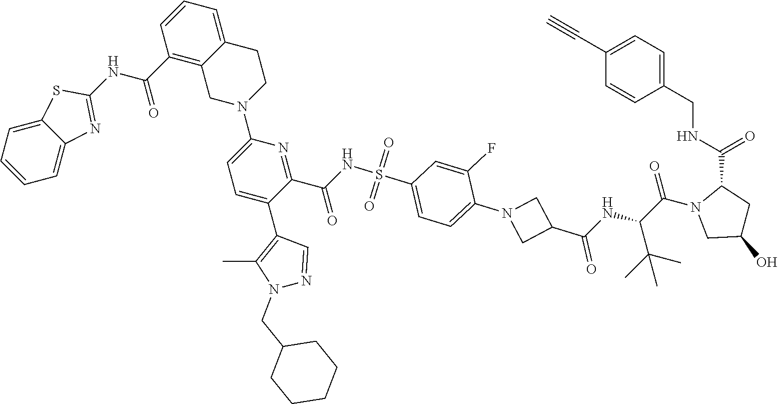 [SMILES: C#Cc1ccc(CNC(=O)[C@@H]2C[C@@H](O)CN2C(=O)[C@@H](NC(=O)C2CN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3F)C2)C(C)(C)C)cc1] |
| I-359 | 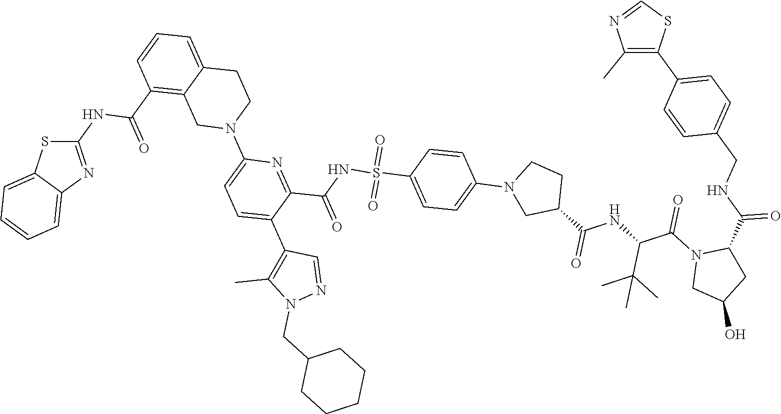 [SMILES: Cc1ncsc1-c1ccc(CNC(=O)[C@@H]2C[C@@H](O)CN2C(=O)[C@@H](NC(=O)[C@H]2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C2)C(C)(C)C)cc1] |
| I-360 | 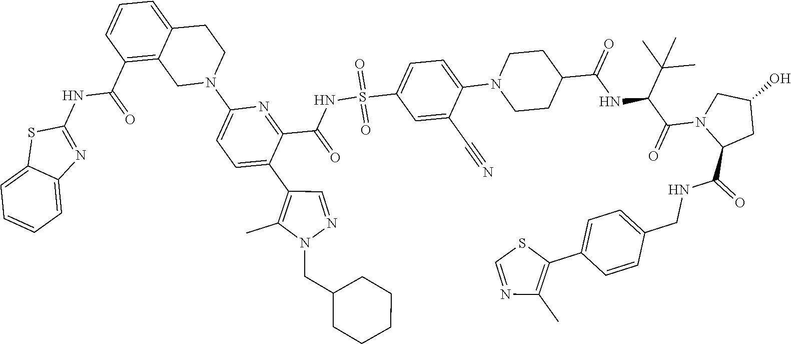 [SMILES: Cc1ncsc1-c1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3C#N)CC2)C(C)(C)C)cc1] |
| I-361 | 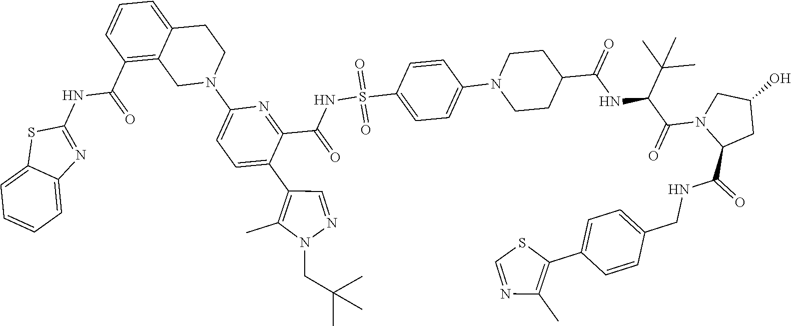 [SMILES: Cc1ncsc1-c1ccc(CNC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC(C)(C)C)c4C)cc3)CC2)C(C)(C)C)cc1] |
| I-362 | 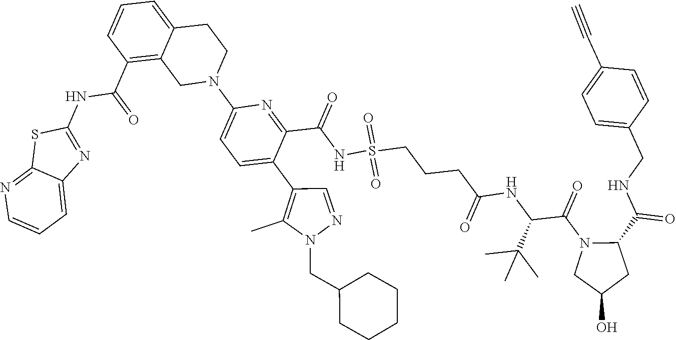 [SMILES: C#Cc1ccc(CNC(=O)[C@@H]2C[C@@H](O)CN2C(=O)[C@@H](NC(=O)CCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-363 | 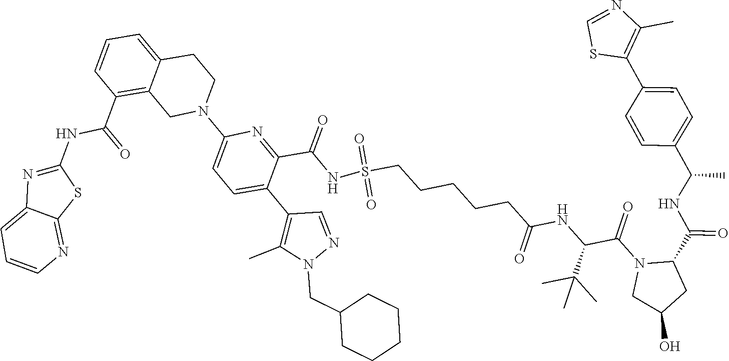 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-364 | 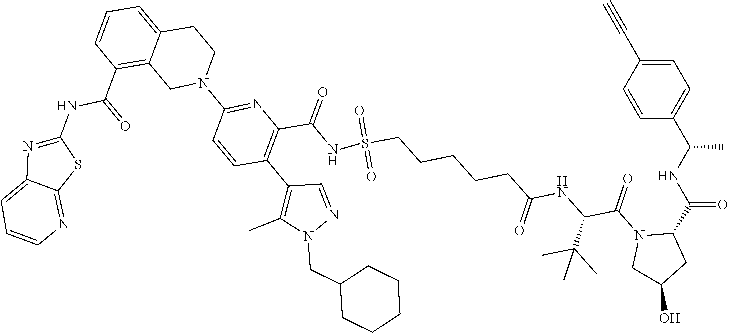 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-365 | 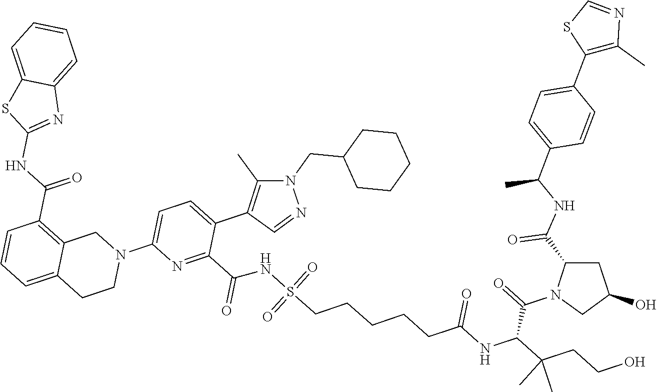 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)CCO)cc1] |
| I-366 | 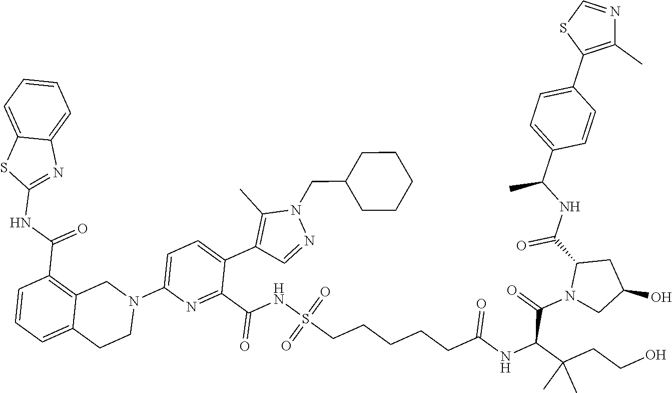 |
| I-367 | 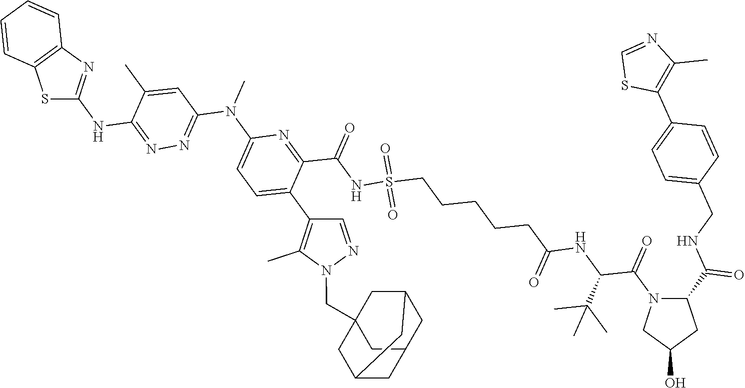 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)NCc3ccc(-c4scnc4C)cc3)C(C)(C)C)n2)nnc1Nc1nc2ccccc2s1] |
| I-368 | 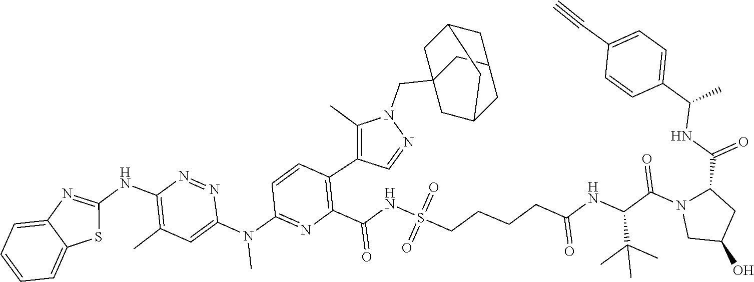 [SMILES: C#Cc1ccc([C@H](C)NC(=O)[C@@H]2C[C@@H](O)CN2C(=O)[C@@H](NC(=O)CCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-369 | 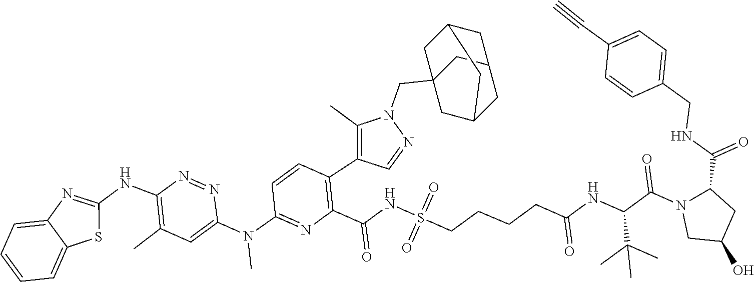 [SMILES: C#Cc1ccc(CNC(=O)[C@@H]2C[C@@H](O)CN2C(=O)[C@@H](NC(=O)CCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-370 | 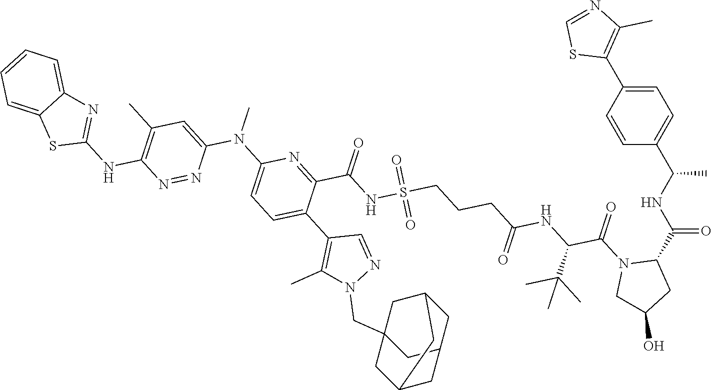 [SMILES: Cc1cc(N(C)c2ccc(-c3cnn(CC45CC6CC(CC(C6)C4)C5)c3C)c(C(=O)NS(=O)(=O)CCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-371 | 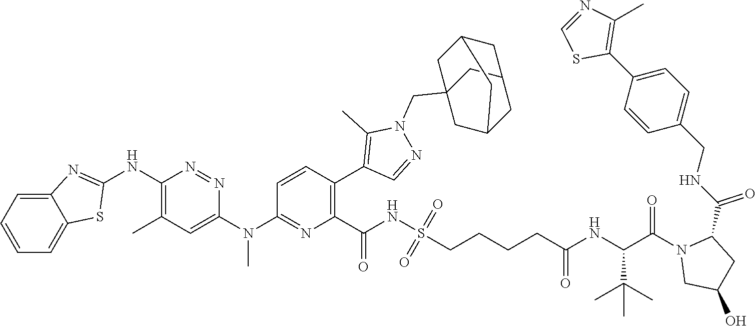 [SMILES: Cc1cc(N(C)c2ccc(-c3cnn(CC45CC6CC(CC(C6)C4)C5)c3C)c(C(=O)NS(=O)(=O)CCCCC(=O)N[C@H](C(=O)N3C[C@H](O)C[C@H]3C(=O)NCc3ccc(-c4scnc4C)cc3)C(C)(C)C)n2)nnc1Nc1nc2ccccc2s1] |
| I-372 | 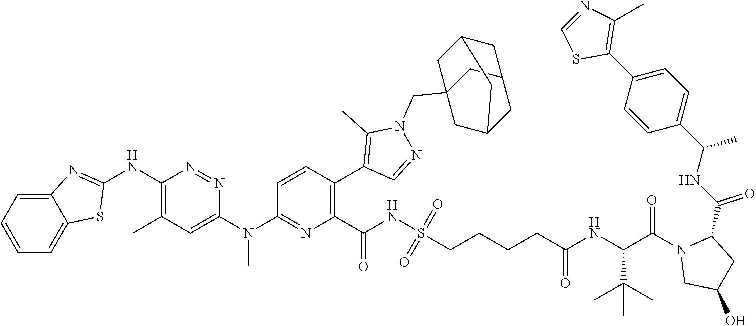 [SMILES: Cc1cc(N(C)c2ccc(-c3cnn(CC45CC6CC(CC(C6)C4)C5)c3C)c(C(=O)NS(=O)(=O)CCCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-373 | 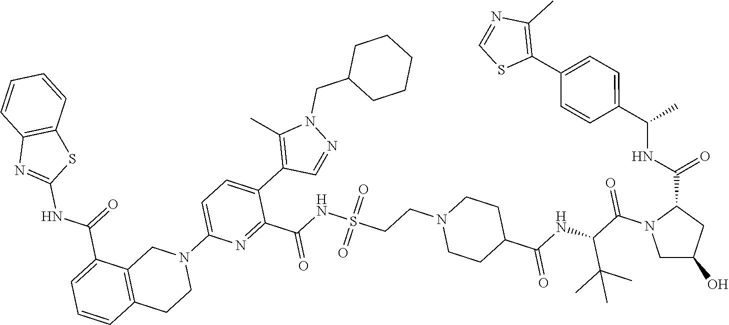 [SMILES: Cc1ncsc1-c1ccc([C@H](C)NC(=O)[C@@H]2C[C@@H](O)CN2C(=O)[C@@H](NC(=O)C2CCN(CCS(=O)(=O)NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-374 | 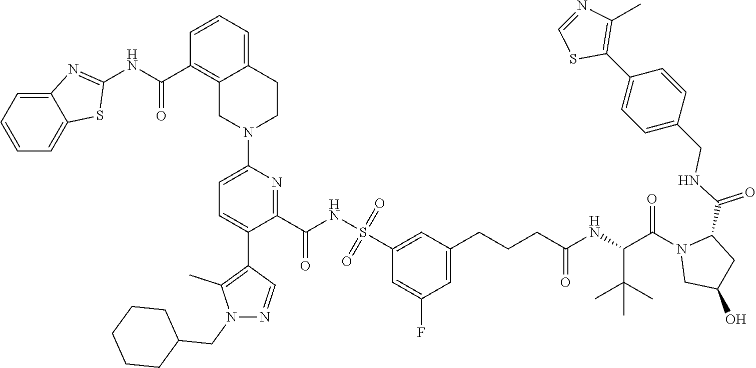 [SMILES: Cc1ncsc1-c1ccc(CNC(=O)[C@@H]2C[C@@H](O)CN2C(=O)[C@@H](NC(=O)CCCc2cc(F)cc(S(=O)(=O)NC(=O)c3nc(N4CCc5cccc(C(=O)Nc6nc7ccccc7s6)c5C4)ccc3-c3cnn(CC4CCCCC4)c3C)c2)C(C)(C)C)cc1] |
| I-375 | 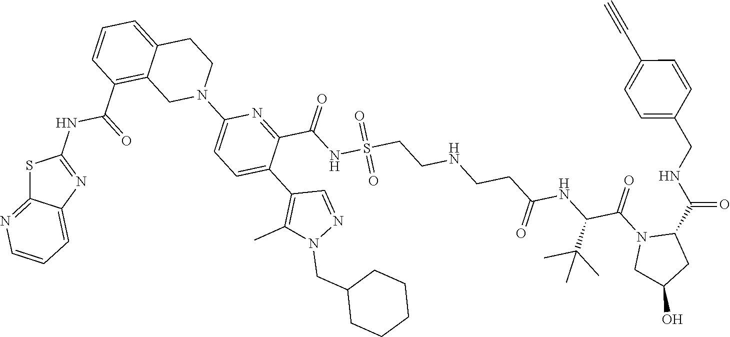 [SMILES: C#Cc1ccc(CNC(=O)[C@@H]2C[C@@H](O)CN2C(=O)[C@@H](NC(=O)CCNCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-376 | 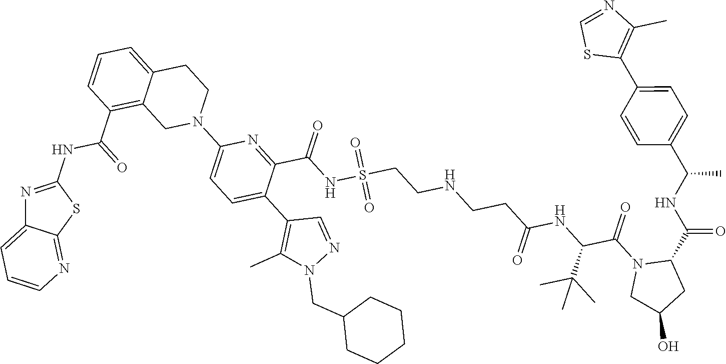 [SMILES: Cc1ncsc1-c1ccc([C@H](C)NC(=O)[C@@H]2C[C@@H](O)CN2C(=O)[C@@H](NC(=O)CCNCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-377 | 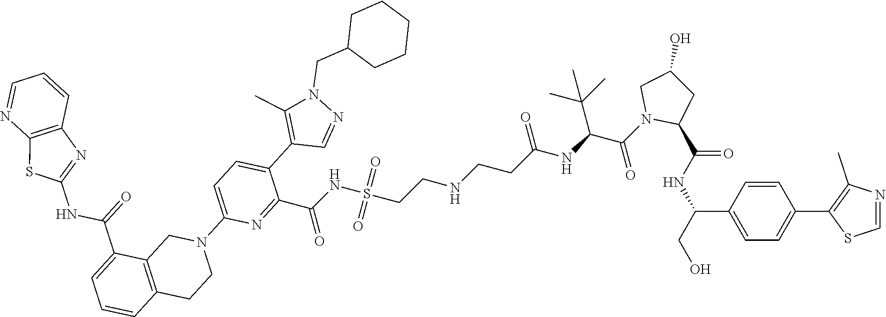 [SMILES: Cc1ncsc1-c1ccc([C@H](CO)NC(=O)[C@@H]2C[C@@H](O)CN2C(=O)[C@@H](NC(=O)CCNCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-378 | 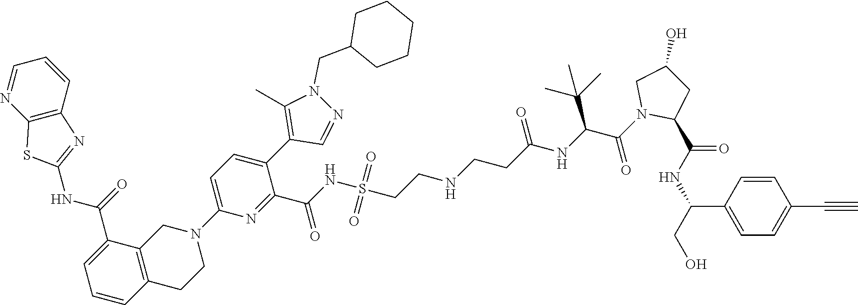 [SMILES: C#Cc1ccc([C@H](CO)NC(=O)[C@@H]2C[C@@H](O)CN2C(=O)[C@@H](NC(=O)CCNCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-379 | 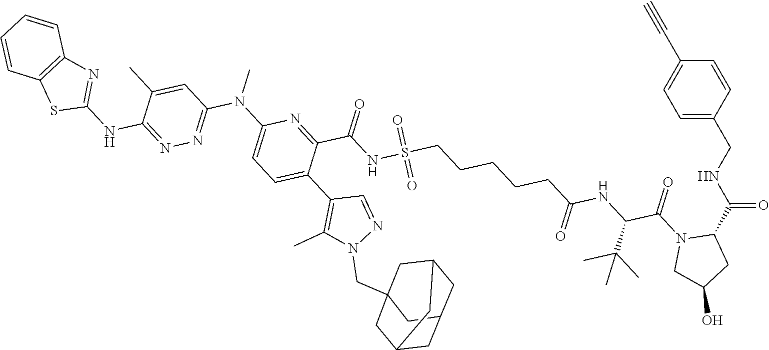 [SMILES: C#Cc1ccc(CNC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-380 | 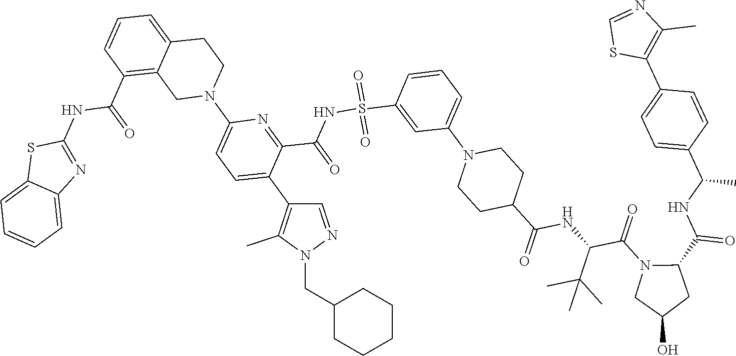 [SMILES: Cc1ncsc1-c1ccc([C@H](C)NC(=O)[C@@H]2C[C@@H](O)CN2C(=O)[C@@H](NC(=O)C2CCN(c3cccc(S(=O)(=O)NC(=O)c4nc(N5CCc6cccc(C(=O)Nc7nc8ccccc8s7)c6C5)ccc4-c4cnn(CC5CCCCC5)c4C)c3)CC2)C(C)(C)C)cc1] |
| I-381 | 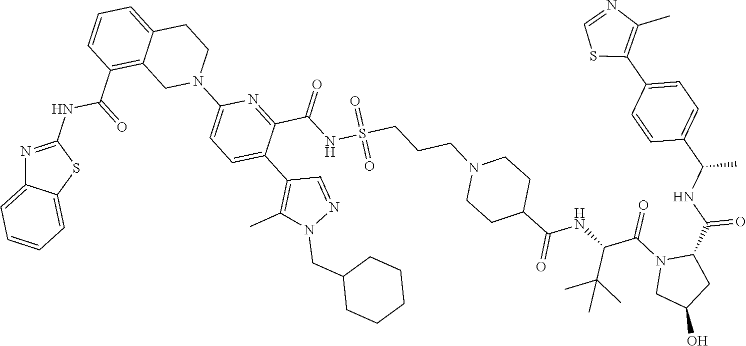 [SMILES: Cc1ncsc1-c1ccc([C@H](C)NC(=O)[C@@H]2C[C@@H](O)CN2C(=O)[C@@H](NC(=O)C2CCN(CCCS(=O)(=O)NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-382 | 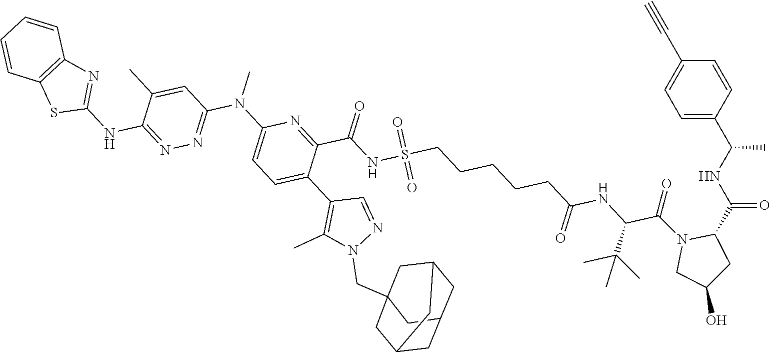 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-383 | 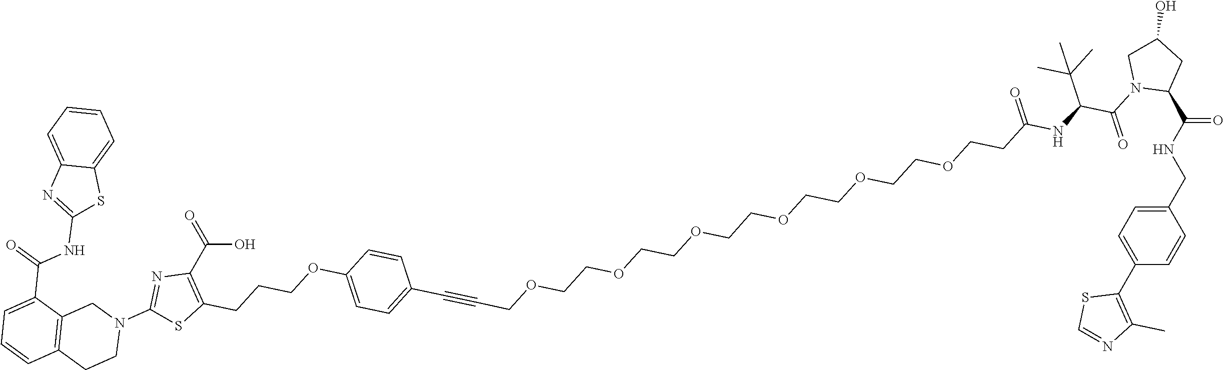 [SMILES: Cc1ncsc1-c1ccc(CNC(=O)[C@@H]2C[C@@H](O)CN2C(=O)[C@@H](NC(=O)CCOCCOCCOCCOCCOCCOCC#Cc2ccc(OCCCc3sc(N4CCc5cccc(C(=O)Nc6nc7ccccc7s6)c5C4)nc3C(=O)O)cc2)C(C)(C)C)cc1] |
| I-384 | 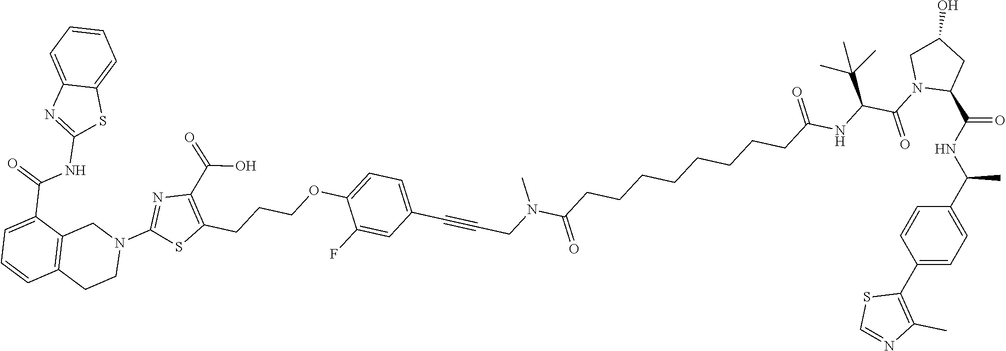 [SMILES: Cc1ncsc1-c1ccc([C@H](C)NC(=O)[C@@H]2C[C@@H](O)CN2C(=O)[C@@H](NC(=O)CCCCCCCCC(=O)N(C)CC#Cc2ccc(OCCCc3sc(N4CCc5cccc(C(=O)Nc6nc7ccccc7s6)c5C4)nc3C(=O)O)c(F)c2)C(C)(C)C)cc1] |
| I-385 | 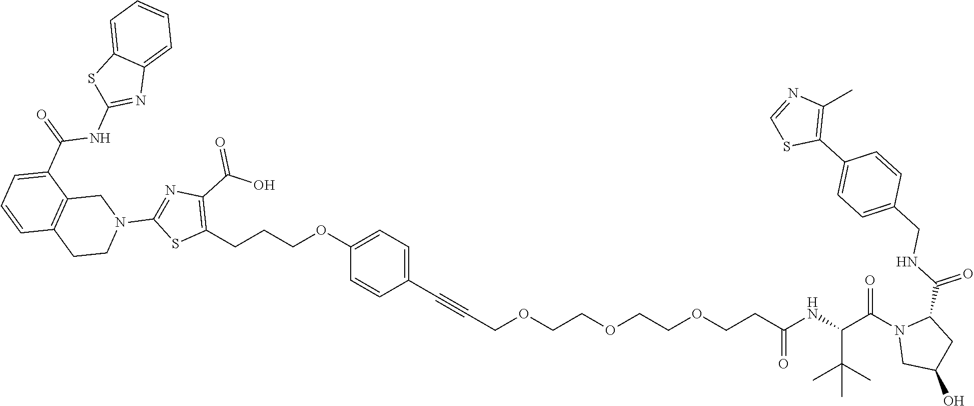 [SMILES: Cc1ncsc1-c1ccc(CNC(=O)[C@@H]2C[C@@H](O)CN2C(=O)[C@@H](NC(=O)CCOCCOCCOCC#Cc2ccc(OCCCc3sc(N4CCc5cccc(C(=O)Nc6nc7ccccc7s6)c5C4)nc3C(=O)O)cc2)C(C)(C)C)cc1] |
| I-386 | 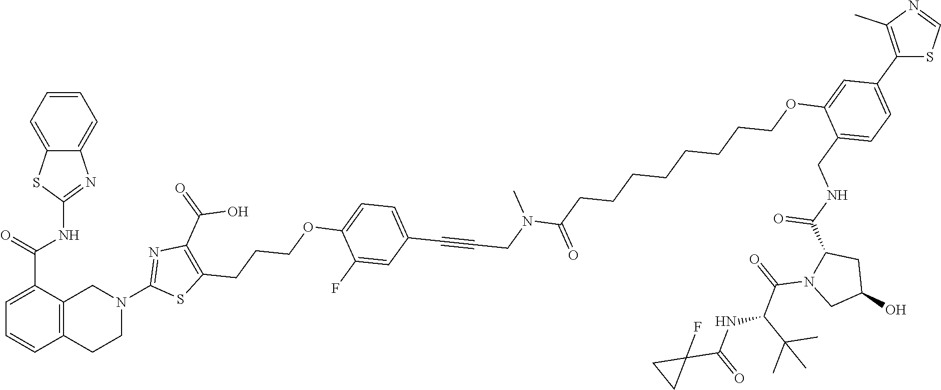 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCC(=O)N(C)CC#Cc2ccc(OCCCc3sc(N4CCc5cccc(C(=O)Nc6nc7ccccc7s6)c5C4)nc3C(=O)O)c(F)c2)c1] |
| I-387 | 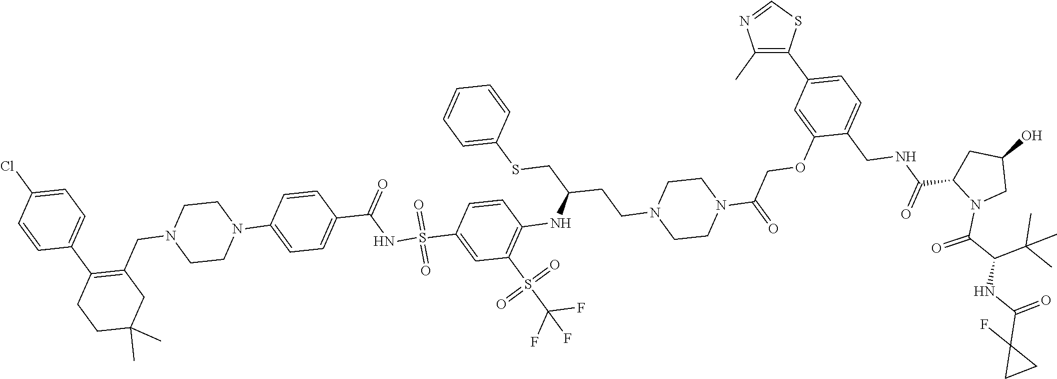 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-388 | 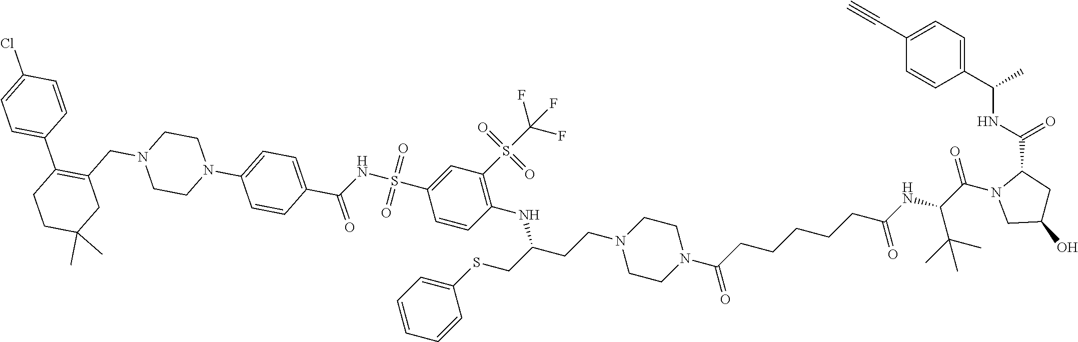 [SMILES: C#Cc1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-389 | 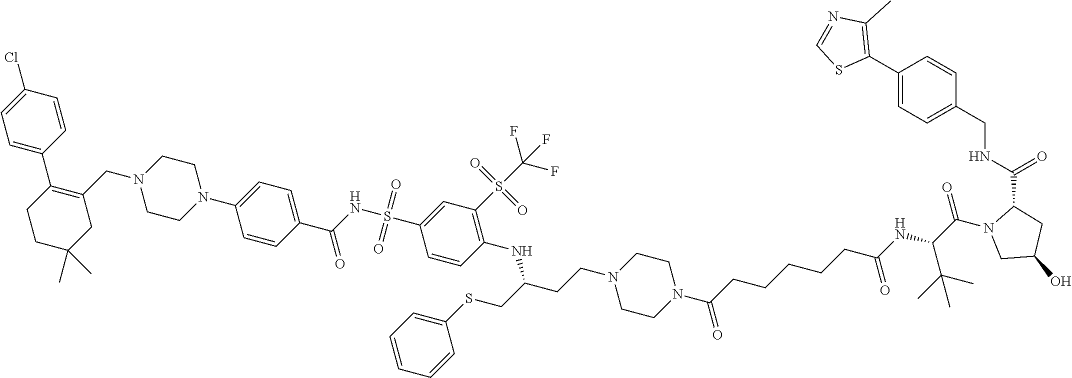 [SMILES: Cc1ncsc1-c1ccc(CNC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-390 | 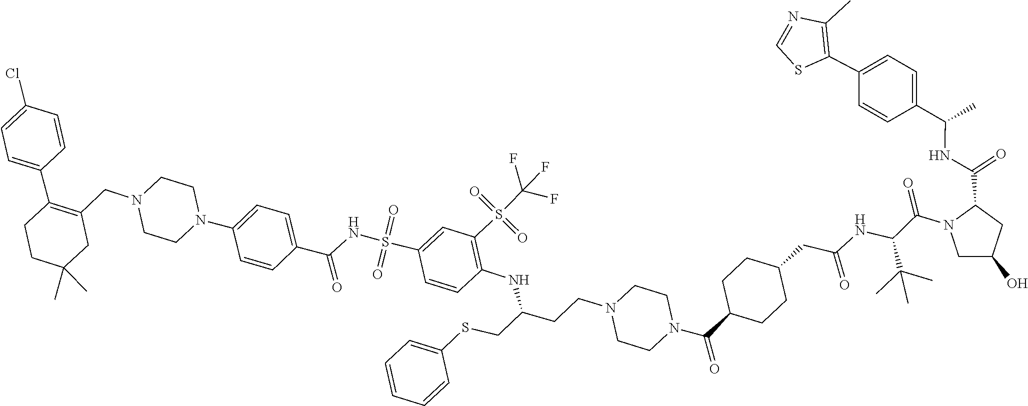 [SMILES: Cc1ncsc1-c1ccc([C@H](C)NC(=O)[C@@H]2C[C@@H](O)CN2C(=O)[C@@H](NC(=O)C[C@H]2CC[C@H](C(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)C(C)(C)C)cc1] |
| I-391 | 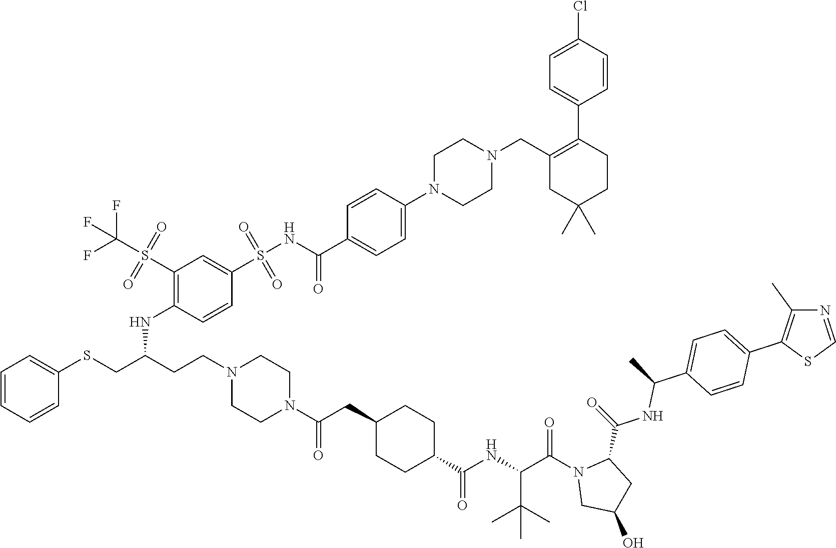 [SMILES: Cc1ncsc1-c1ccc([C@H](C)NC(=O)[C@@H]2C[C@@H](O)CN2C(=O)[C@@H](NC(=O)[C@H]2CC[C@H](CC(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)C(C)(C)C)cc1] |
| I-392 | 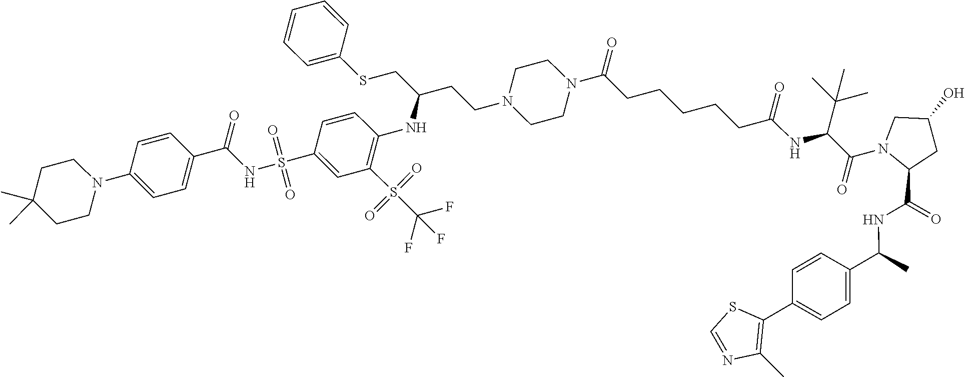 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC(C)(C)CC5)cc4)cc3S(=O)(=O)C(F)(F)F)CC2)C(C)(C)C)cc1] |
| I-393 | 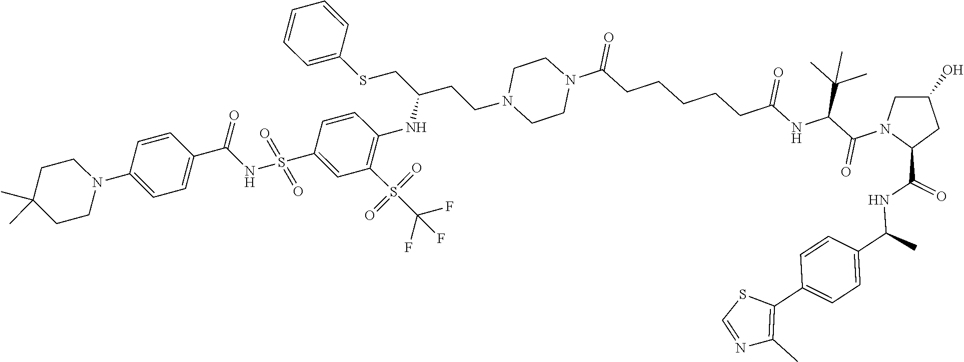 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@@H](CSc3ccccc3)Nc3ccc(S(=O)(=O)NC(=O)c4ccc(N5CCC(C)(C)CC5)cc4)cc3S(=O)(=O)C(F)(F)F)CC2)C(C)(C)C)cc1] |
| I-394 | 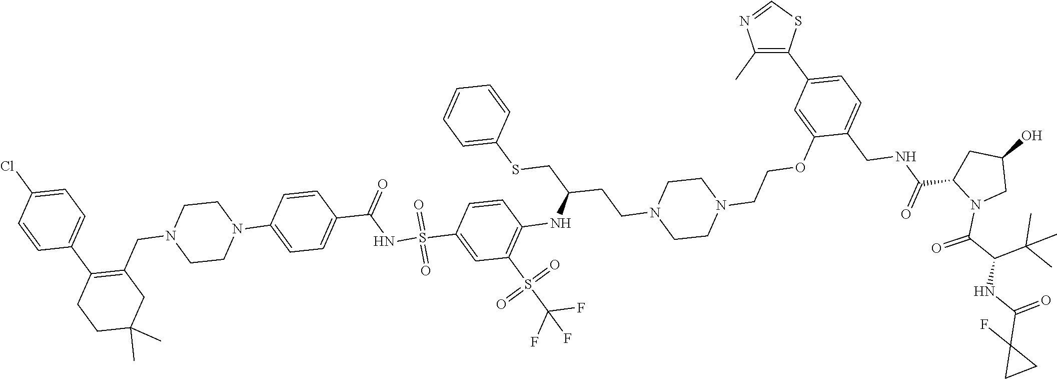 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-395 | 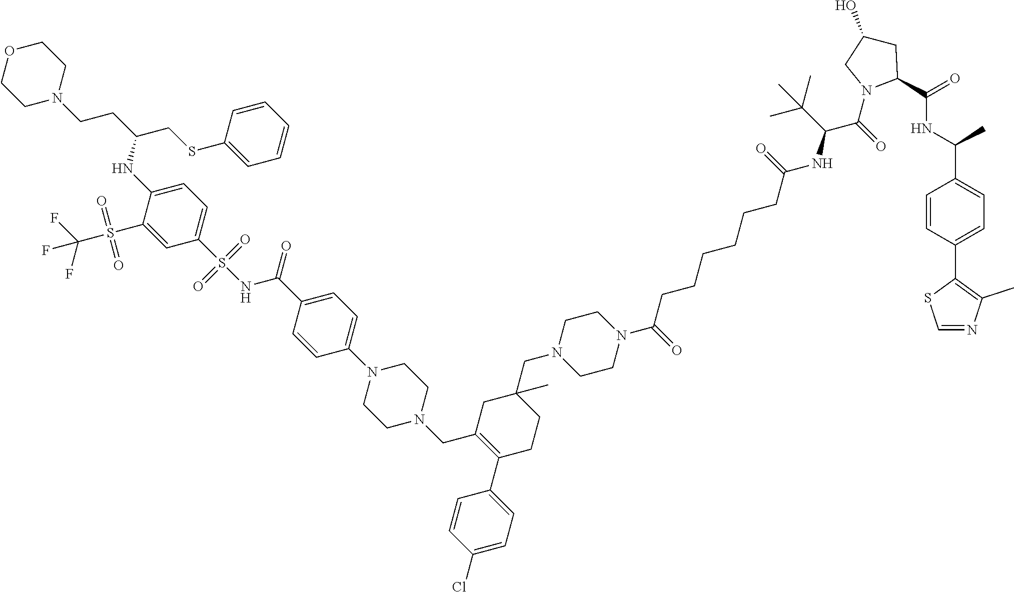 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| I-396 | 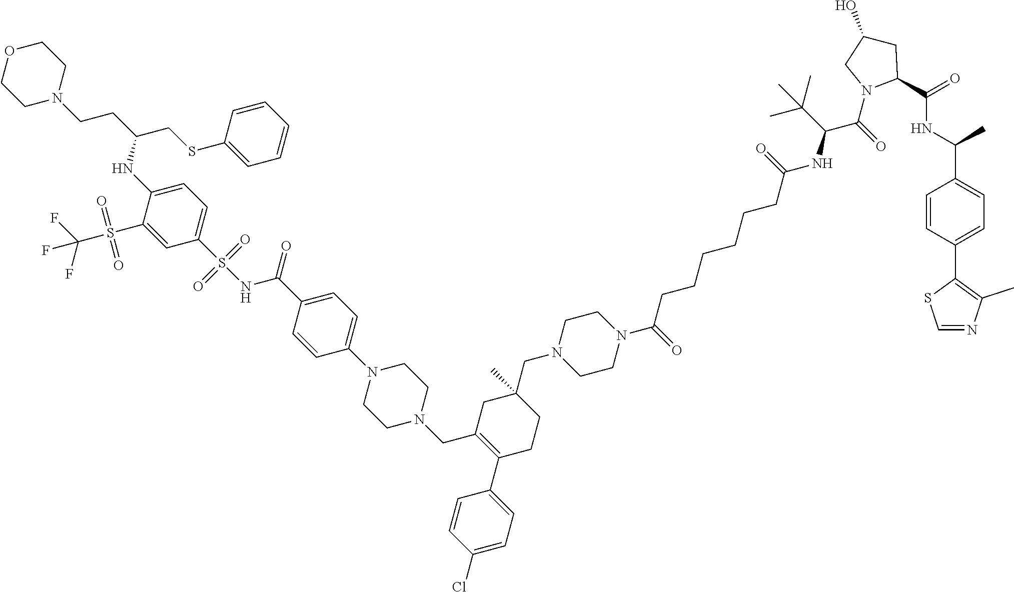 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| I-397 | 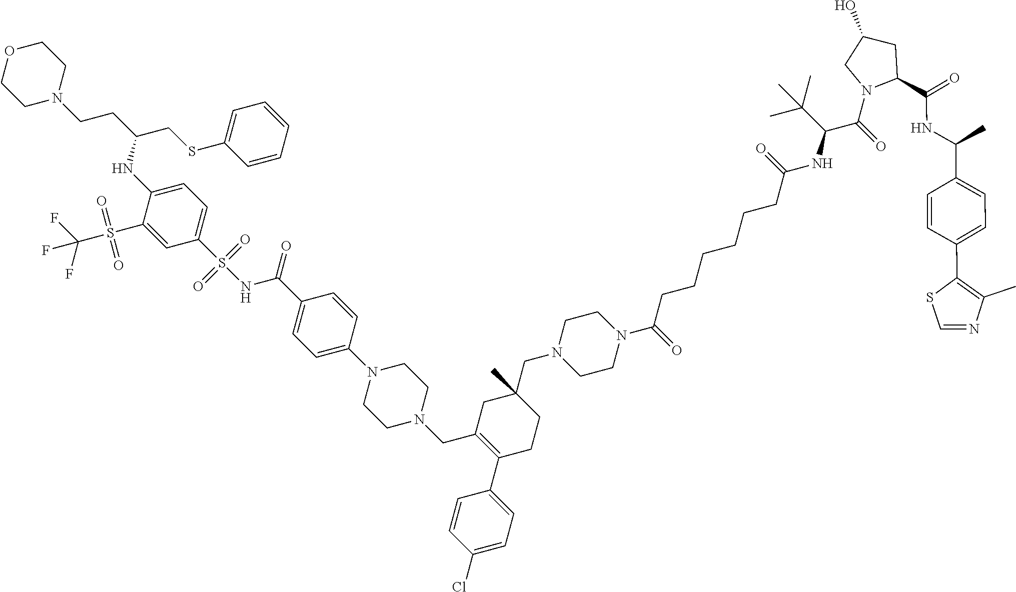 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| I-398 | 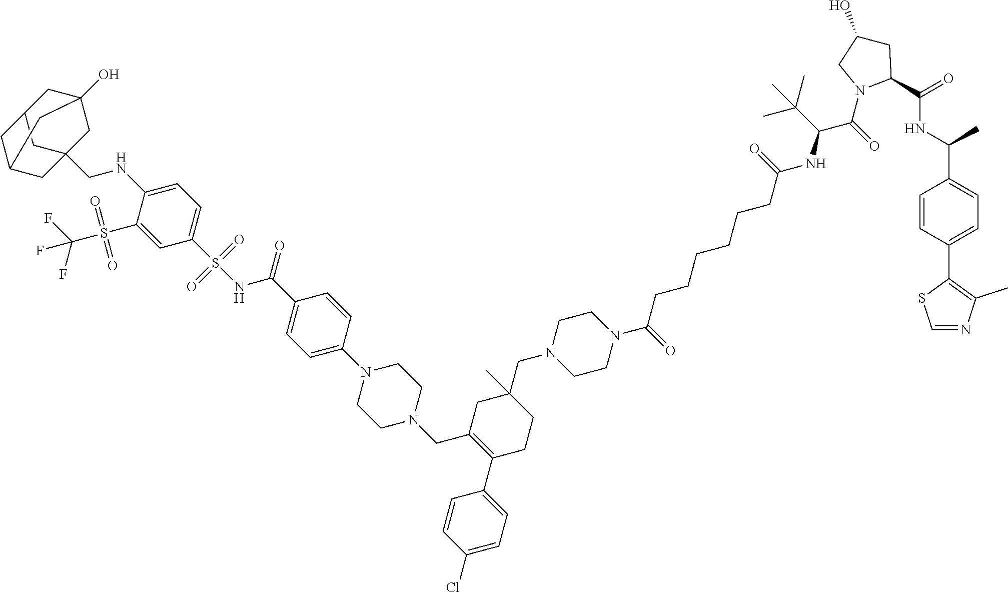 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(NCC78CC9CC(CC(O)(C9)C7)C8)c(S(=O)(=O)C(F)(F)F)c6)cc5)CC4)C3)CC2)C(C)(C)C)cc1] |
| I-399 | 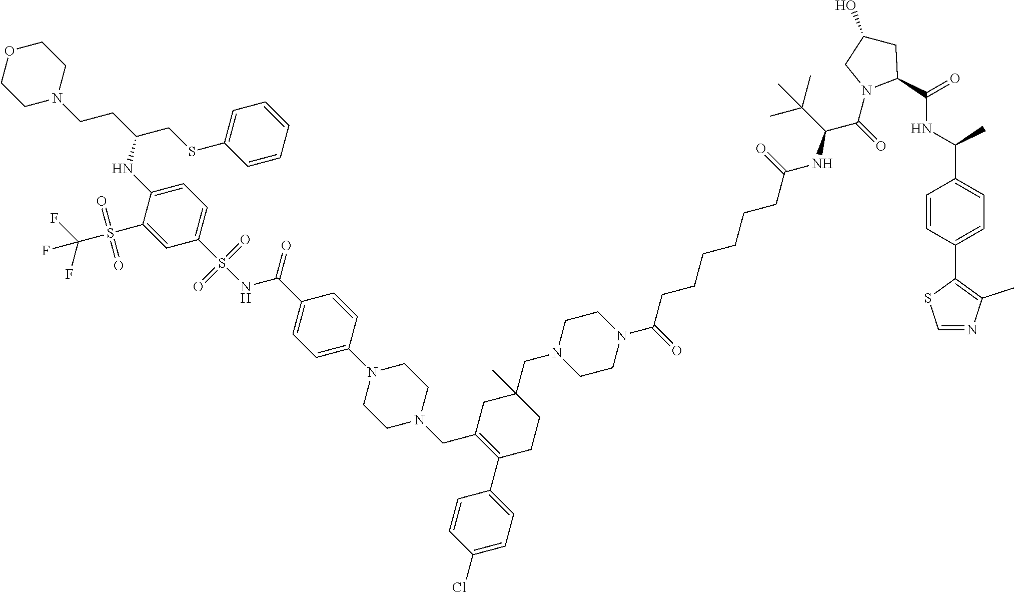 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| I-400 | 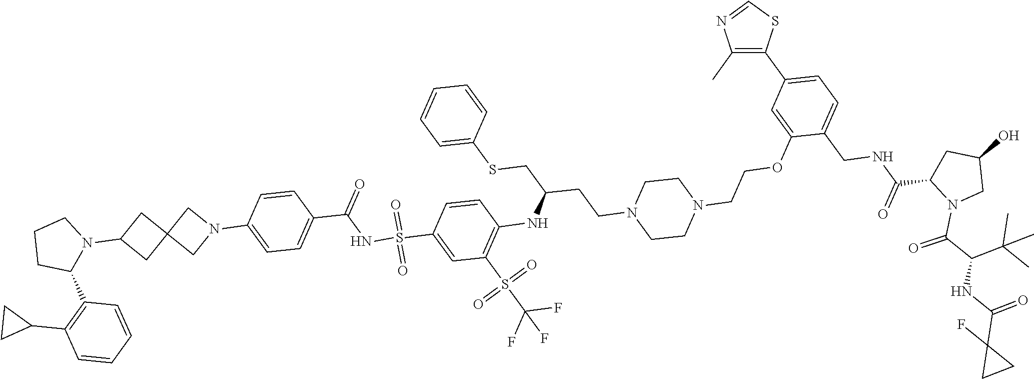 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@H](CSc3ccccc3)Nc3ccc(S(=O)(=O)NC(=O)c4ccc(N5CC6(CC(N7CCC[C@H]7c7ccccc7C7CC7)C6)C5)cc4)cc3S(=O)(=O)C(F)(F)F)CC2)c1] |
| I-407 | 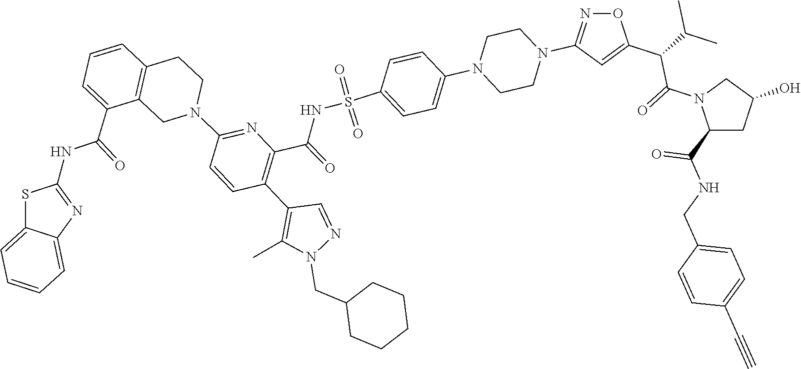 [SMILES: C#Cc1ccc(CNC(=O)[C@@H]2C[C@@H](O)CN2C(=O)[C@H](c2cc(N3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)cc1] |
| I-408 | 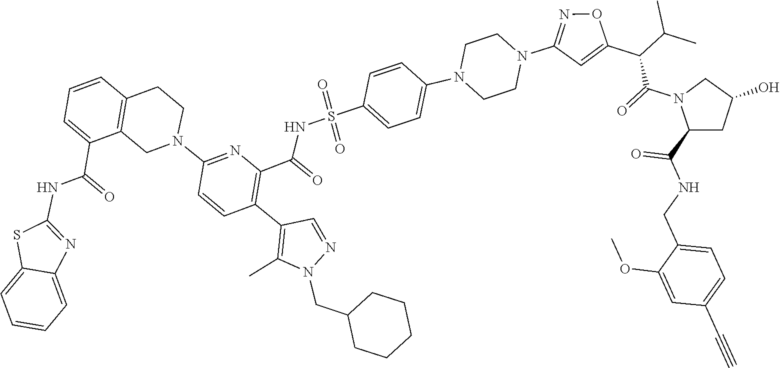 [SMILES: C#Cc1ccc(CNC(=O)[C@@H]2C[C@@H](O)CN2C(=O)[C@H](c2cc(N3CCN(c4ccc(S(=O)(=O)NC(=O)c5nc(N6CCc7cccc(C(=O)Nc8nc9ccccc9s8)c7C6)ccc5-c5cnn(CC6CCCCC6)c5C)cc4)CC3)no2)C(C)C)c(OC)c1] |
| I-409 | 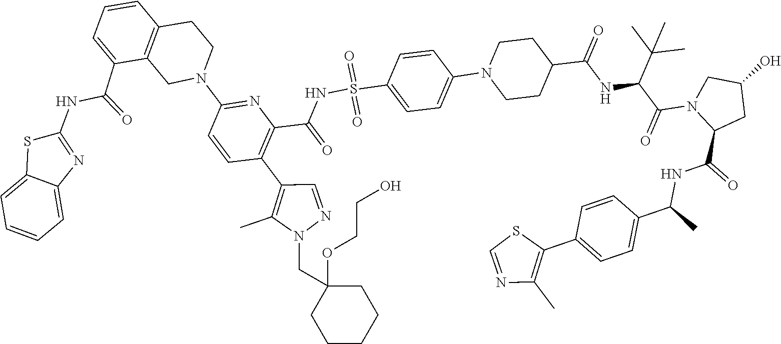 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4nc(N5CCc6cccc(C(=O)Nc7nc8ccccc8s7)c6C5)ccc4-c4cnn(CC5(OCCO)CCCCC5)c4C)cc3)CC2)C(C)(C)C)cc1] |
| I-410 | 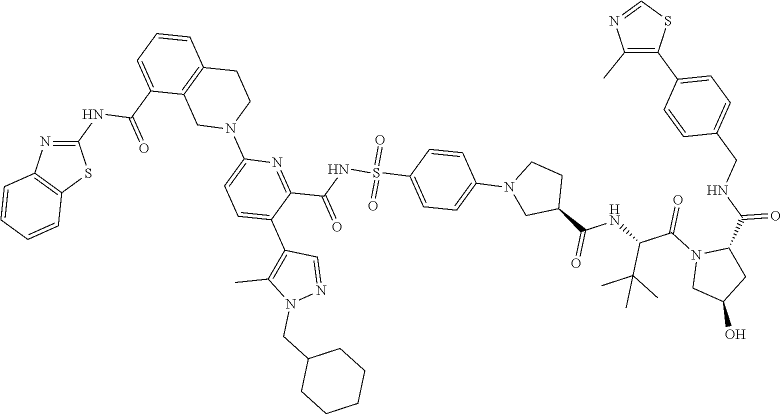 [SMILES: Cc1ncsc1-c1ccc(CNC(=O)[C@@H]2C[C@@H](O)CN2C(=O)[C@@H](NC(=O)[C@@H]2CCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C2)C(C)(C)C)cc1] |
| I-411 | 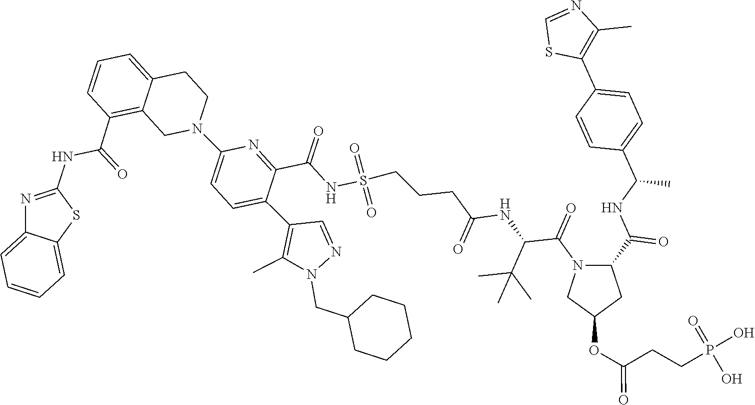 [SMILES: Cc1ncsc1-c1ccc([C@H](C)NC(=O)[C@@H]2C[C@@H](OC(=O)CCP(=O)(O)O)CN2C(=O)[C@@H](NC(=O)CCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-412 | 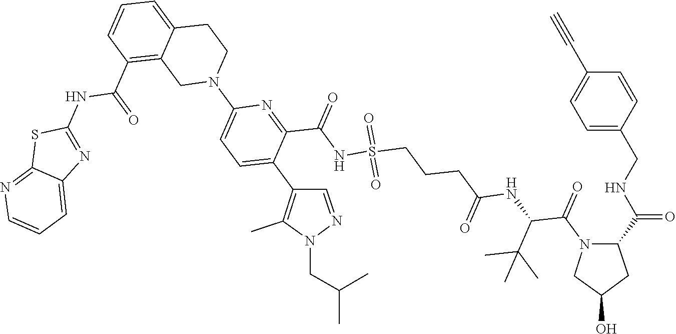 [SMILES: C#Cc1ccc(CNC(=O)[C@@H]2C[C@@H](O)CN2C(=O)[C@@H](NC(=O)CCCS(=O)(=O)NC(=O)c2nc(N3CCc4cccc(C(=O)Nc5nc6cccnc6s5)c4C3)ccc2-c2cnn(CC(C)C)c2C)C(C)(C)C)cc1] |
| I-413 | 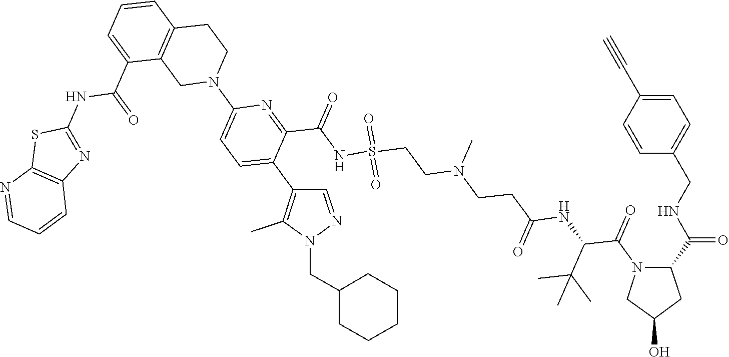 [SMILES: C#Cc1ccc(CNC(=O)[C@@H]2C[C@@H](O)CN2C(=O)[C@@H](NC(=O)CCN(C)CCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-414 | 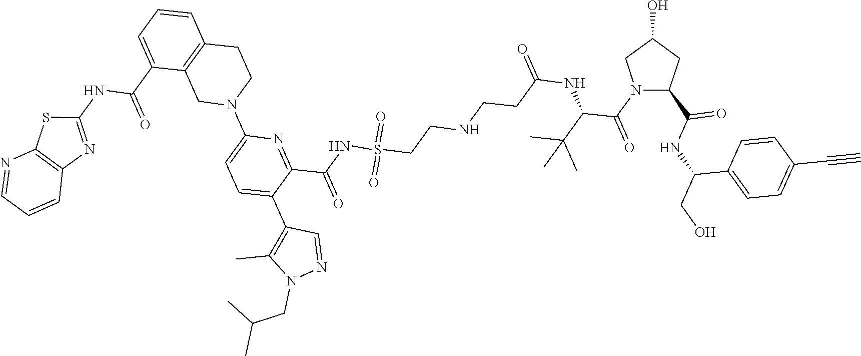 [SMILES: C#Cc1ccc([C@H](CO)NC(=O)[C@@H]2C[C@@H](O)CN2C(=O)[C@@H](NC(=O)CCNCCS(=O)(=O)NC(=O)c2nc(N3CCc4cccc(C(=O)Nc5nc6cccnc6s5)c4C3)ccc2-c2cnn(CC(C)C)c2C)C(C)(C)C)cc1] |
| I-415 | 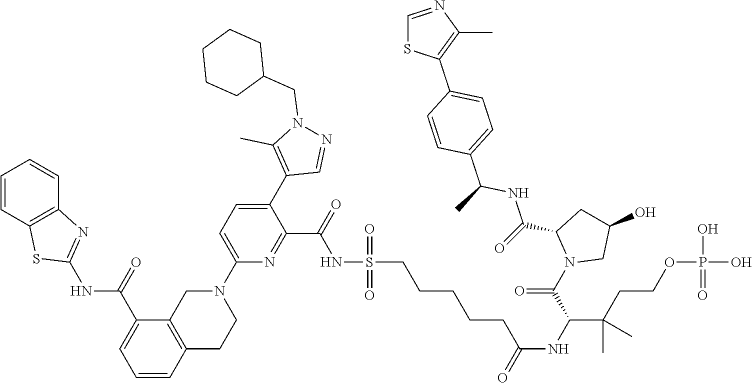 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)CCOP(=O)(O)O)cc1] |
| I-416 | 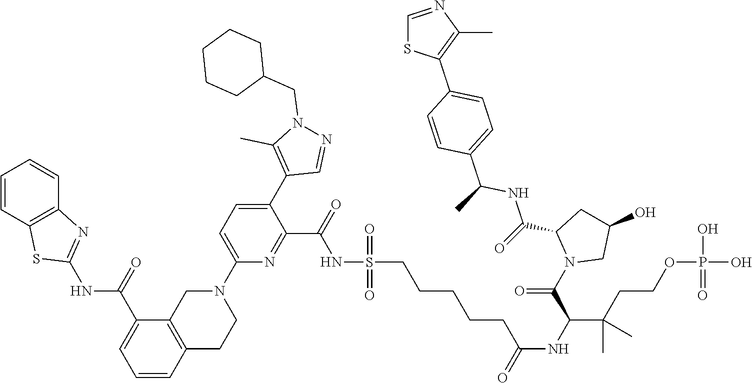 [SMILES: Cc1ncsc1-c1ccc([C@H](C)NC(=O)[C@@H]2C[C@@H](O)CN2C(=O)[C@H](NC(=O)CCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)CCOP(=O)(O)O)cc1] |
| I-417 | 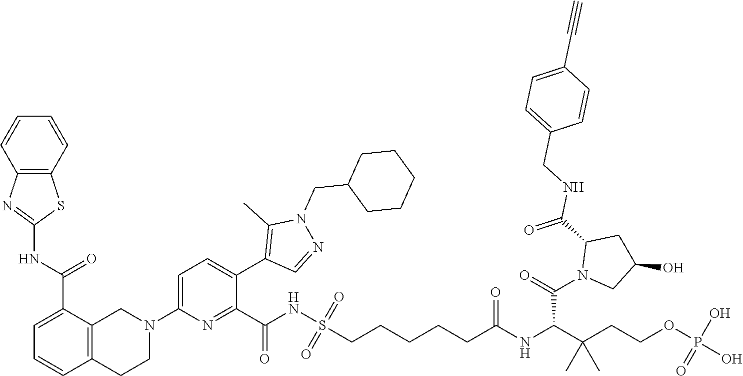 [SMILES: C#Cc1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)CCOP(=O)(O)O)cc1] |
| I-418 | 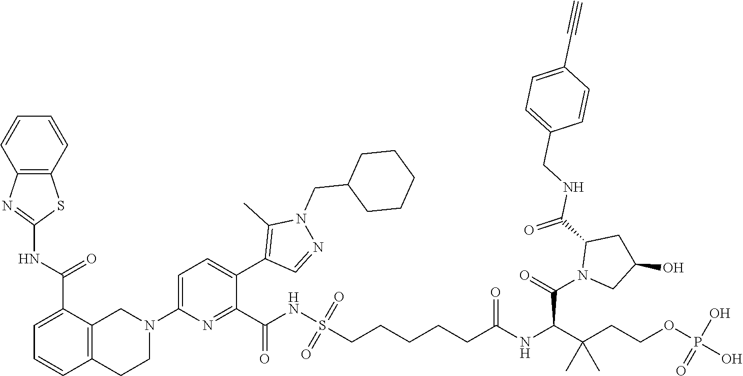 [SMILES: C#Cc1ccc(CNC(=O)[C@@H]2C[C@@H](O)CN2C(=O)[C@H](NC(=O)CCCCCS(=O)(=O)NC(=O)c2nc(N3CCc4cccc(C(=O)Nc5nc6ccccc6s5)c4C3)ccc2-c2cnn(CC3CCCCC3)c2C)C(C)(C)CCOP(=O)(O)O)cc1] |
| I-419 | 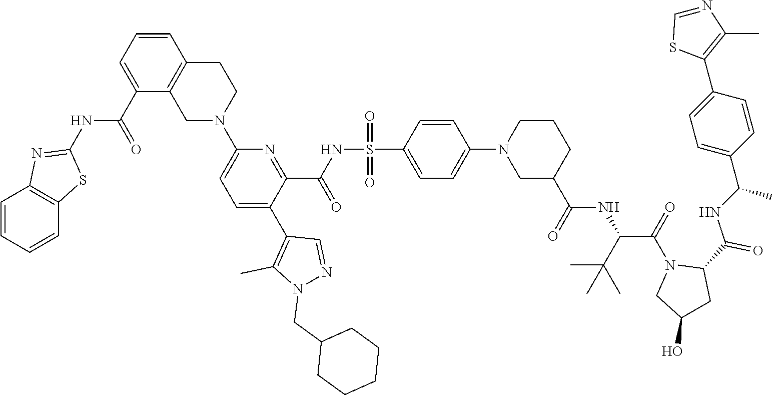 [SMILES: Cc1ncsc1-c1ccc([C@H](C)NC(=O)[C@@H]2C[C@@H](O)CN2C(=O)[C@@H](NC(=O)C2CCCN(c3ccc(S(=O)(=O)NC(=O)c4nc(N5CCc6cccc(C(=O)Nc7nc8ccccc8s7)c6C5)ccc4-c4cnn(CC5CCCCC5)c4C)cc3)C2)C(C)(C)C)cc1] |
| I-420 | 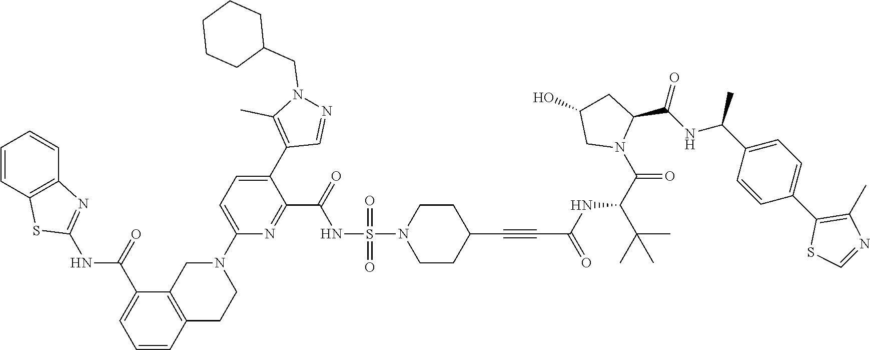 [SMILES: Cc1ncsc1-c1ccc([C@H](C)NC(=O)[C@@H]2C[C@@H](O)CN2C(=O)[C@@H](NC(=O)C#CC2CCN(S(=O)(=O)NC(=O)c3nc(N4CCc5cccc(C(=O)Nc6nc7ccccc7s6)c5C4)ccc3-c3cnn(CC4CCCCC4)c3C)CC2)C(C)(C)C)cc1] |
| I-421 | 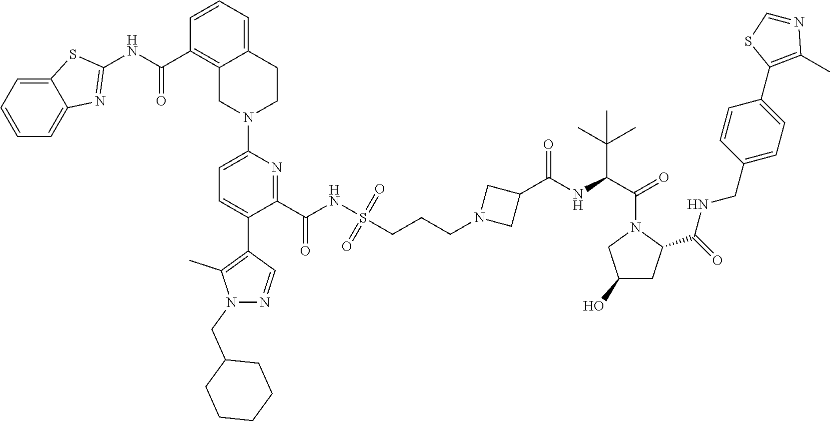 [SMILES: Cc1ncsc1-c1ccc(CNC(=O)[C@@H]2C[C@@H](O)CN2C(=O)[C@@H](NC(=O)C2CN(CCCS(=O)(=O)NC(=O)c3nc(N4CCc5cccc(C(=O)Nc6nc7ccccc7s6)c5C4)ccc3-c3cnn(CC4CCCCC4)c3C)C2)C(C)(C)C)cc1] |
| I-422 | 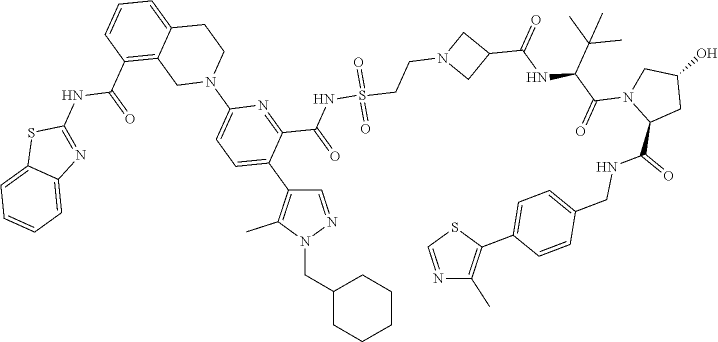 [SMILES: Cc1ncsc1-c1ccc(CNC(=O)[C@@H]2C[C@@H](O)CN2C(=O)[C@@H](NC(=O)C2CN(CCS(=O)(=O)NC(=O)c3nc(N4CCc5cccc(C(=O)Nc6nc7ccccc7s6)c5C4)ccc3-c3cnn(CC4CCCCC4)c3C)C2)C(C)(C)C)cc1] |
| I-423 | 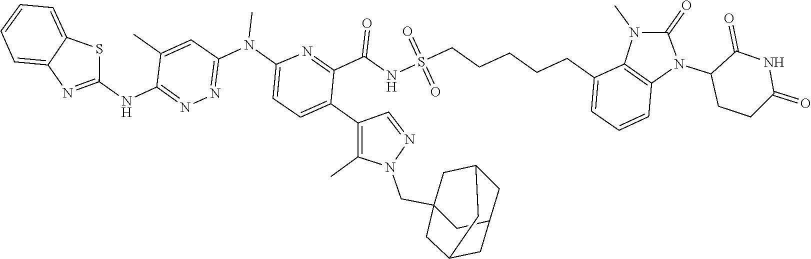 [SMILES: Cc1cc(N(C)c2ccc(-c3cnn(CC45CC6CC(CC(C6)C4)C5)c3C)c(C(=O)NS(=O)(=O)CCCCCc3cccc4c3n(C)c(=O)n4C3CCC(=O)NC3=O)n2)nnc1Nc1nc2ccccc2s1] |
| I-424 | 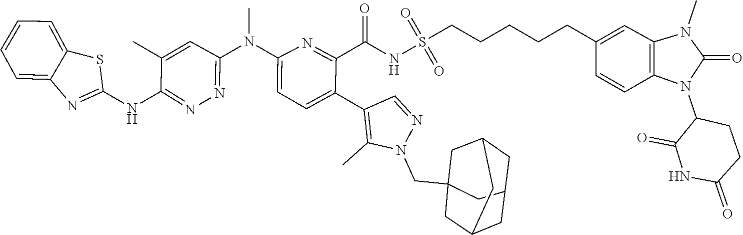 [SMILES: Cc1cc(N(C)c2ccc(-c3cnn(CC45CC6CC(CC(C6)C4)C5)c3C)c(C(=O)NS(=O)(=O)CCCCCc3ccc4c(c3)n(C)c(=O)n4C3CCC(=O)NC3=O)n2)nnc1Nc1nc2ccccc2s1] |
| I-425 | 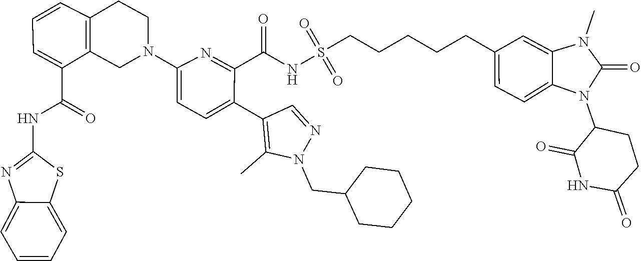 [SMILES: Cc1c(-c2ccc(N3CCc4cccc(C(=O)Nc5nc6ccccc6s5)c4C3)nc2C(=O)NS(=O)(=O)CCCCCc2ccc3c(c2)n(C)c(=O)n3C2CCC(=O)NC2=O)cnn1CC1CCCCC1] |
| I-426 | 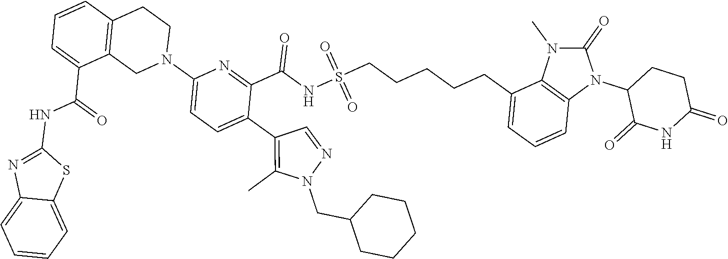 [SMILES: Cc1c(-c2ccc(N3CCc4cccc(C(=O)Nc5nc6ccccc6s5)c4C3)nc2C(=O)NS(=O)(=O)CCCCCc2cccc3c2n(C)c(=O)n3C2CCC(=O)NC2=O)cnn1CC1CCCCC1] |
| I-427 | 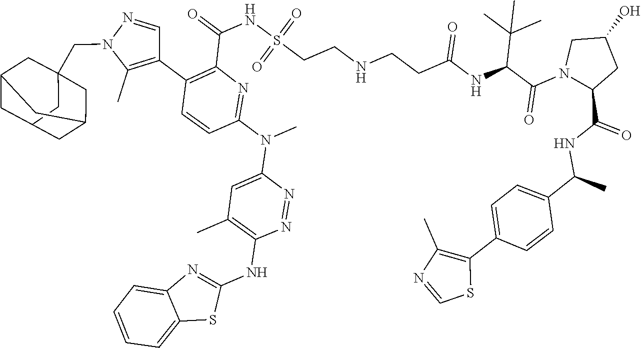 [SMILES: Cc1cc(N(C)c2ccc(-c3cnn(CC45CC6CC(CC(C6)C4)C5)c3C)c(C(=O)NS(=O)(=O)CCNCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-428 | 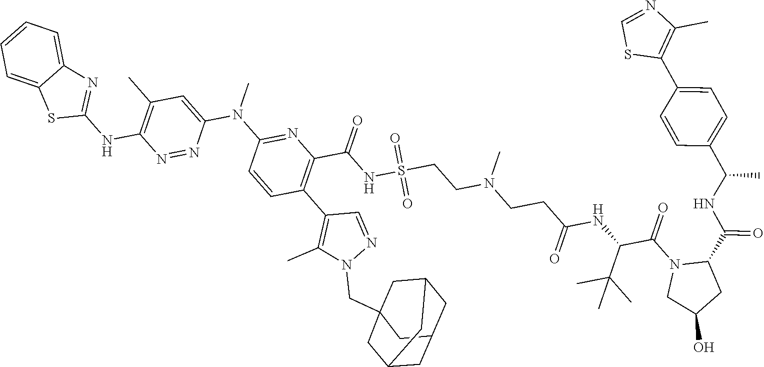 [SMILES: Cc1cc(N(C)c2ccc(-c3cnn(CC45CC6CC(CC(C6)C4)C5)c3C)c(C(=O)NS(=O)(=O)CCN(C)CCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-429 | 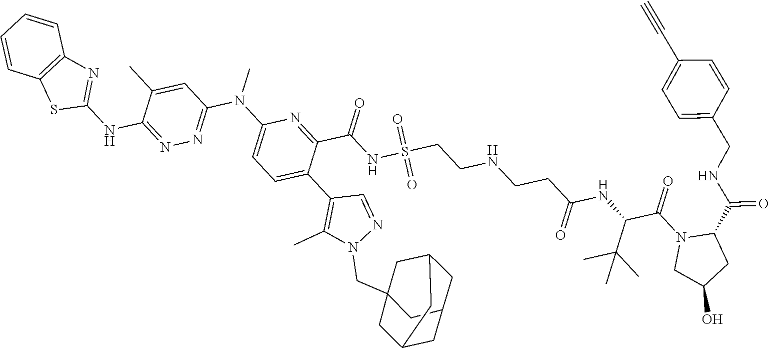 [SMILES: C#Cc1ccc(CNC(=O)[C@@H]2C[C@@H](O)CN2C(=O)[C@@H](NC(=O)CCNCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-430 | 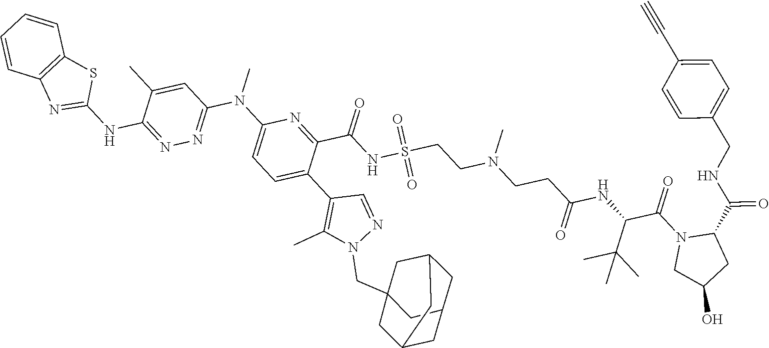 [SMILES: C#Cc1ccc(CNC(=O)[C@@H]2C[C@@H](O)CN2C(=O)[C@@H](NC(=O)CCN(C)CCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-431 | 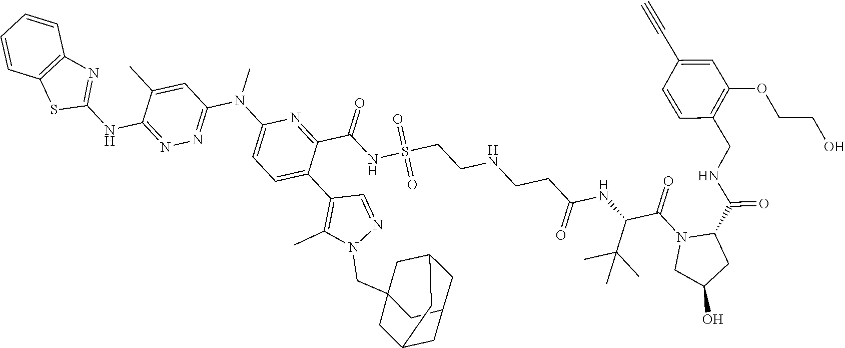 [SMILES: C#Cc1ccc(CNC(=O)[C@@H]2C[C@@H](O)CN2C(=O)[C@@H](NC(=O)CCNCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)c(OCCO)c1] |
| I-432 | 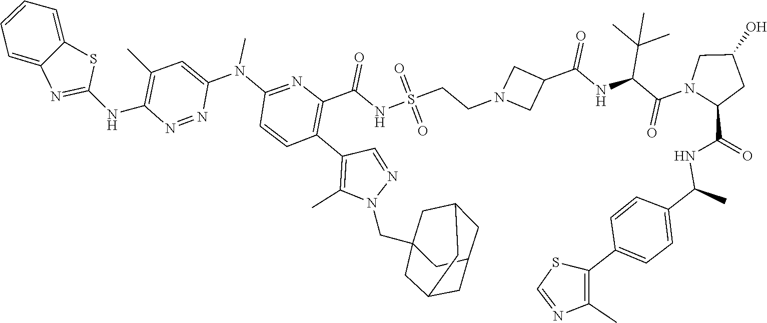 [SMILES: Cc1cc(N(C)c2ccc(-c3cnn(CC45CC6CC(CC(C6)C4)C5)c3C)c(C(=O)NS(=O)(=O)CCN3CC(C(=O)N[C@H](C(=O)N4C[C@H](O)C[C@H]4C(=O)N[C@@H](C)c4ccc(-c5scnc5C)cc4)C(C)(C)C)C3)n2)nnc1Nc1nc2ccccc2s1] |
| I-433 | 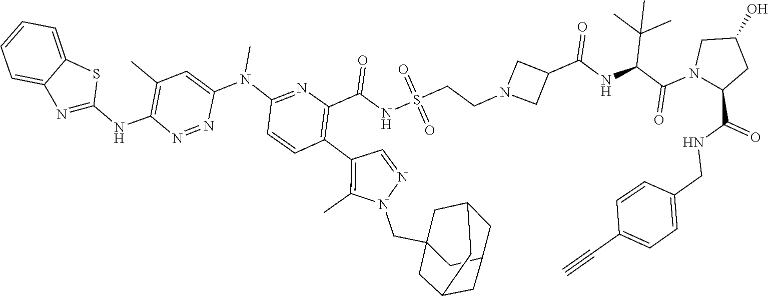 [SMILES: C#Cc1ccc(CNC(=O)[C@@H]2C[C@@H](O)CN2C(=O)[C@@H](NC(=O)C2CN(CCS(=O)(=O)NC(=O)c3nc(N(C)c4cc(C)c(Nc5nc6ccccc6s5)nn4)ccc3-c3cnn(CC45CC6CC(CC(C6)C4)C5)c3C)C2)C(C)(C)C)cc1] |
| I-434 | 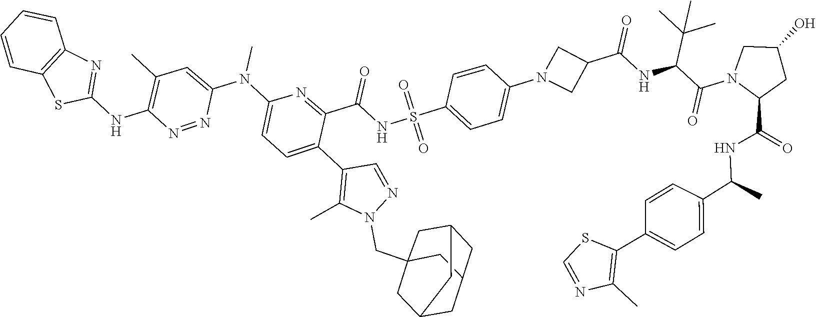 [SMILES: Cc1cc(N(C)c2ccc(-c3cnn(CC45CC6CC(CC(C6)C4)C5)c3C)c(C(=O)NS(=O)(=O)c3ccc(N4CC(C(=O)N[C@H](C(=O)N5C[C@H](O)C[C@H]5C(=O)N[C@@H](C)c5ccc(-c6scnc6C)cc5)C(C)(C)C)C4)cc3)n2)nnc1Nc1nc2ccccc2s1] |
| I-435 | 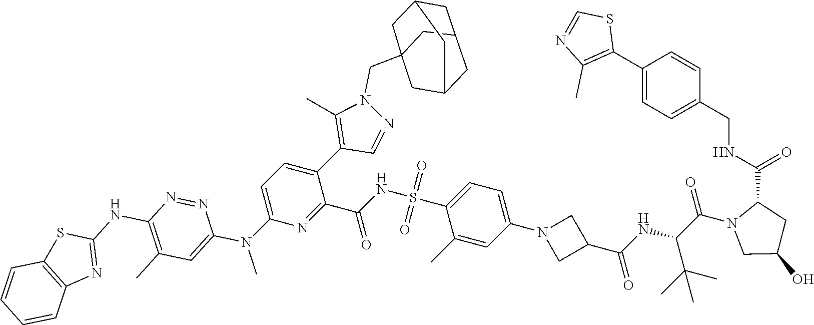 [SMILES: Cc1cc(N2CC(C(=O)N[C@H](C(=O)N3C[C@H](O)C[C@H]3C(=O)NCc3ccc(-c4scnc4C)cc3)C(C)(C)C)C2)ccc1S(=O)(=O)NC(=O)c1nc(N(C)c2cc(C)c(Nc3nc4ccccc4s3)nn2)ccc1-c1cnn(CC23CC4CC(CC(C4)C2)C3)c1C] |
| I-436 | 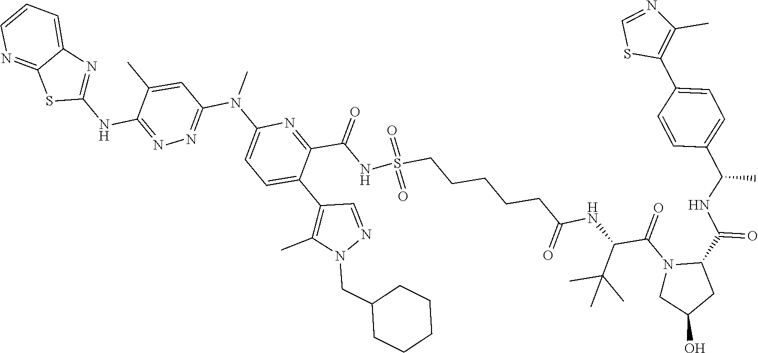 [SMILES: Cc1cc(N(C)c2ccc(-c3cnn(CC4CCCCC4)c3C)c(C(=O)NS(=O)(=O)CCCCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2cccnc2s1] |
| I-437 | 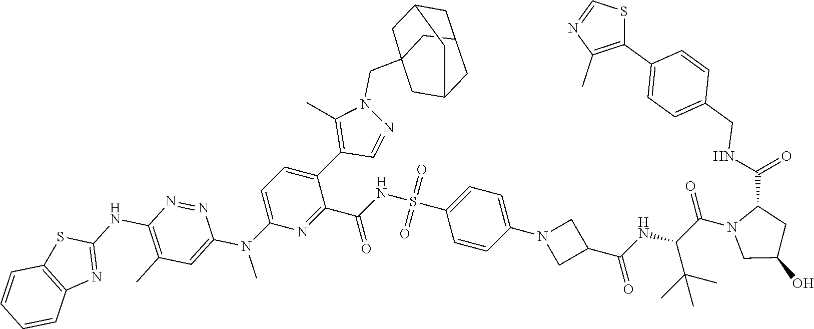 [SMILES: Cc1cc(N(C)c2ccc(-c3cnn(CC45CC6CC(CC(C6)C4)C5)c3C)c(C(=O)NS(=O)(=O)c3ccc(N4CC(C(=O)N[C@H](C(=O)N5C[C@H](O)C[C@H]5C(=O)NCc5ccc(-c6scnc6C)cc5)C(C)(C)C)C4)cc3)n2)nnc1Nc1nc2ccccc2s1] |
| I-438 | 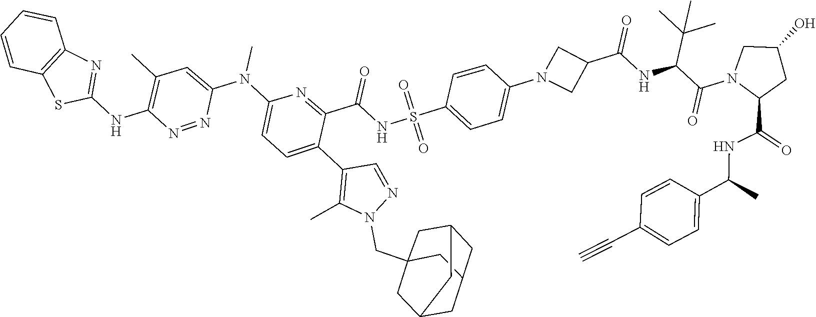 [SMILES: C#Cc1ccc([C@H](C)NC(=O)[C@@H]2C[C@@H](O)CN2C(=O)[C@@H](NC(=O)C2CN(c3ccc(S(=O)(=O)NC(=O)c4nc(N(C)c5cc(C)c(Nc6nc7ccccc7s6)nn5)ccc4-c4cnn(CC56CC7CC(CC(C7)C5)C6)c4C)cc3)C2)C(C)(C)C)cc1] |
| I-439 | 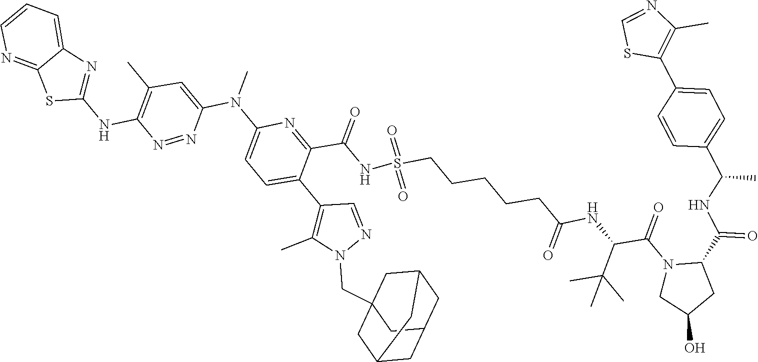 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2cccnc2s1] |
| I-440 | 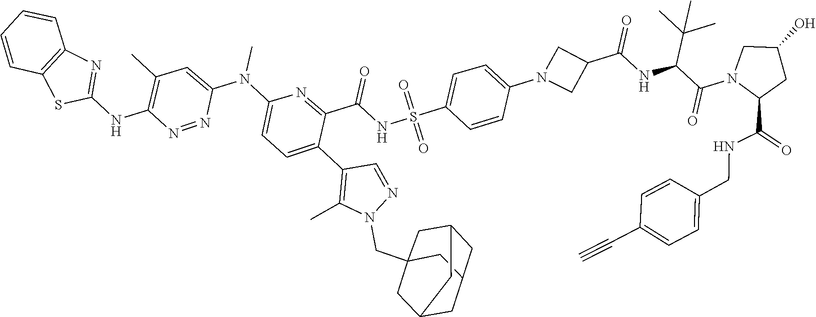 [SMILES: C#Cc1ccc(CNC(=O)[C@@H]2C[C@@H](O)CN2C(=O)[C@@H](NC(=O)C2CN(c3ccc(S(=O)(=O)NC(=O)c4nc(N(C)c5cc(C)c(Nc6nc7ccccc7s6)nn5)ccc4-c4cnn(CC56CC7CC(CC(C7)C5)C6)c4C)cc3)C2)C(C)(C)C)cc1] |
| I-441 | 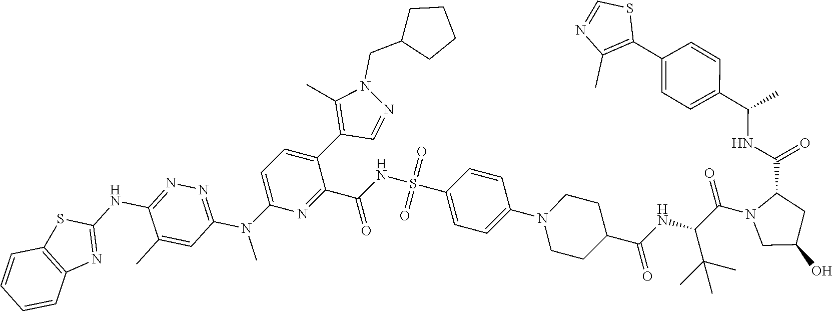 [SMILES: Cc1cc(N(C)c2ccc(-c3cnn(CC4CCCC4)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2ccccc2s1] |
| I-442 | 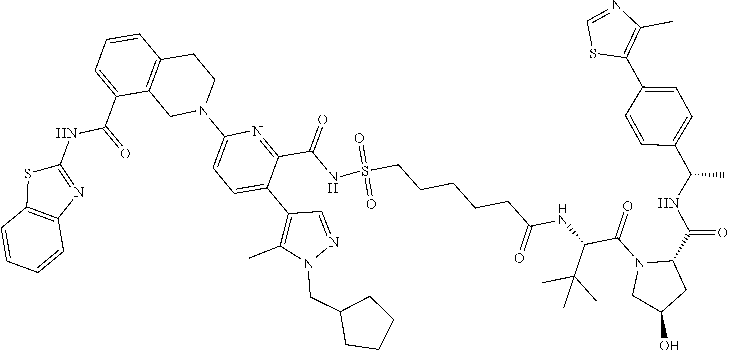 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC3CCCC3)c2C)C(C)(C)C)cc1] |
| I-443 | 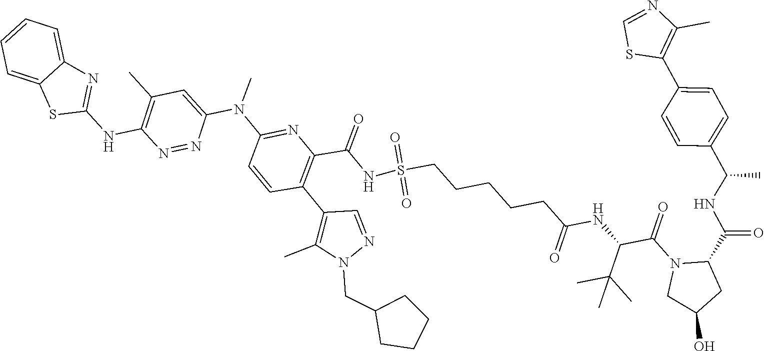 [SMILES: Cc1cc(N(C)c2ccc(-c3cnn(CC4CCCC4)c3C)c(C(=O)NS(=O)(=O)CCCCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2ccccc2s1] |
| I-444 | 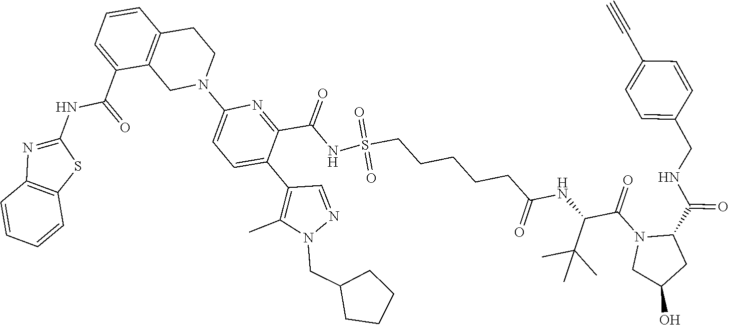 [SMILES: C#Cc1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC3CCCC3)c2C)C(C)(C)C)cc1] |
| I-445 | 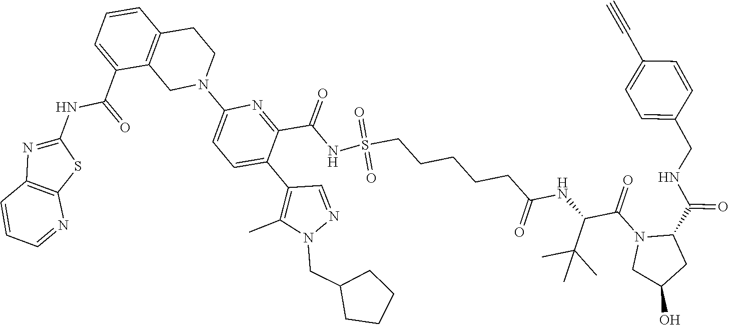 [SMILES: C#Cc1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCC3)c2C)C(C)(C)C)cc1] |
| I-446 | 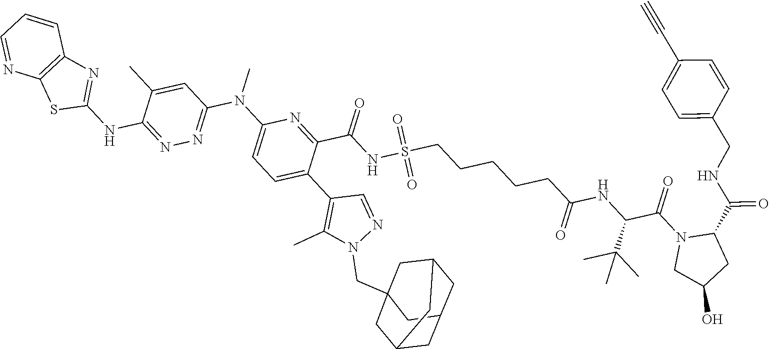 [SMILES: C#Cc1ccc(CNC(=O)[C@@H]2C[C@@H](O)CN2C(=O)[C@@H](NC(=O)CCCCCS(=O)(=O)NC(=O)c2nc(N(C)c3cc(C)c(Nc4nc5cccnc5s4)nn3)ccc2-c2cnn(CC34CC5CC(CC(C5)C3)C4)c2C)C(C)(C)C)cc1] |
| I-447 | 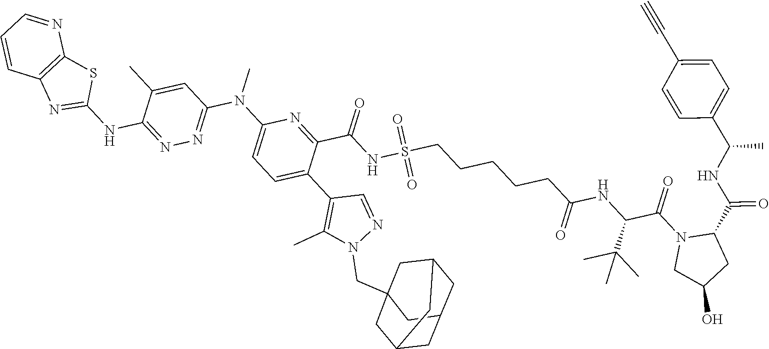 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N(C)c3cc(C)c(Nc4nc5cccnc5s4)nn3)ccc2-c2cnn(CC34CC5CC(CC(C5)C3)C4)c2C)C(C)(C)C)cc1] |
| I-448 | 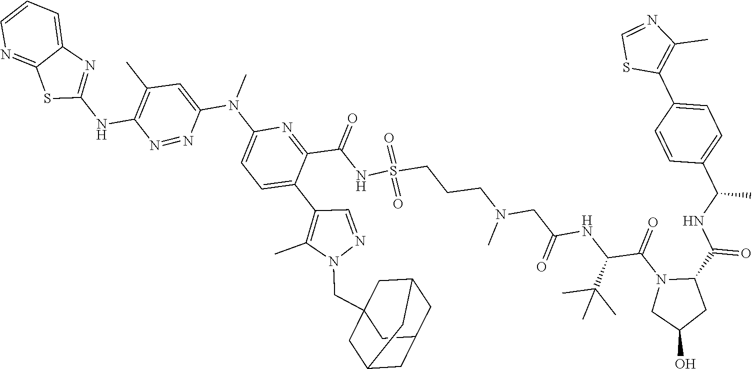 [SMILES: Cc1cc(N(C)c2ccc(-c3cnn(CC45CC6CC(CC(C6)C4)C5)c3C)c(C(=O)NS(=O)(=O)CCCN(C)CC(=O)N[C@H](C(=O)N3C[C@H](O)C[C@H]3C(=O)N[C@@H](C)c3ccc(-c4scnc4C)cc3)C(C)(C)C)n2)nnc1Nc1nc2cccnc2s1] |
| I-449 | 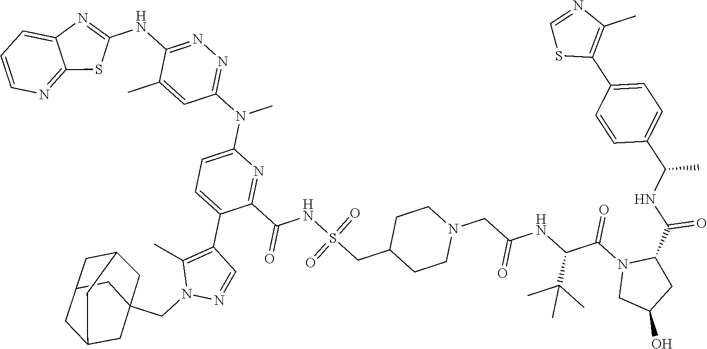 [SMILES: Cc1cc(N(C)c2ccc(-c3cnn(CC45CC6CC(CC(C6)C4)C5)c3C)c(C(=O)NS(=O)(=O)CC3CCN(CC(=O)N[C@H](C(=O)N4C[C@H](O)C[C@H]4C(=O)N[C@@H](C)c4ccc(-c5scnc5C)cc4)C(C)(C)C)CC3)n2)nnc1Nc1nc2cccnc2s1] |
| I-450 | 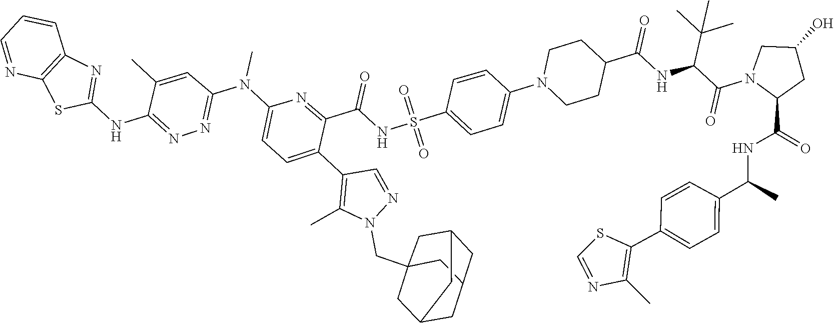 [SMILES: Cc1cc(N(C)c2ccc(-c3cnn(CC45CC6CC(CC(C6)C4)C5)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2cccnc2s1] |
| I-451 | 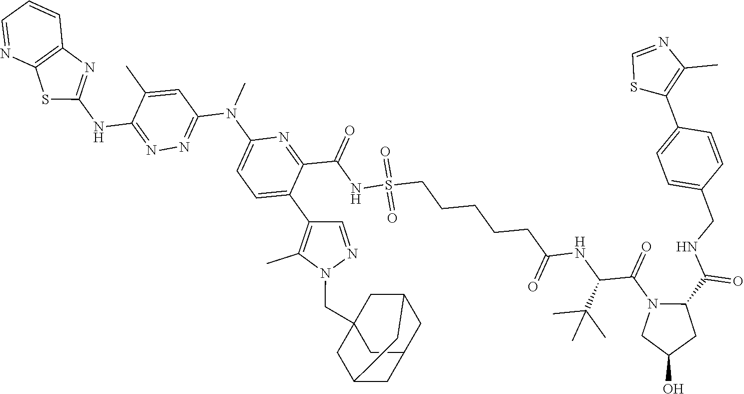 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)NCc3ccc(-c4scnc4C)cc3)C(C)(C)C)n2)nnc1Nc1nc2cccnc2s1] |
| I-452 | 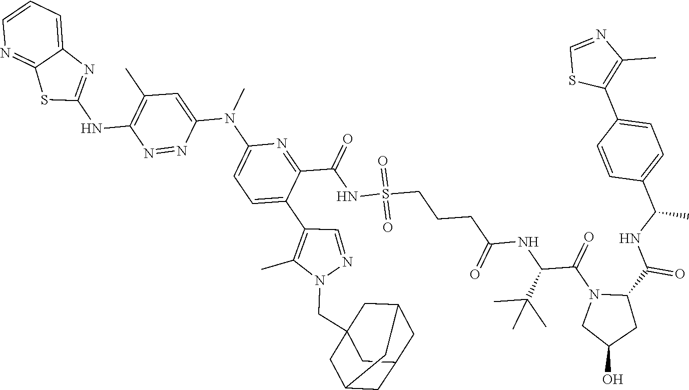 [SMILES: Cc1cc(N(C)c2ccc(-c3cnn(CC45CC6CC(CC(C6)C4)C5)c3C)c(C(=O)NS(=O)(=O)CCCC(=O)N[C@H](C(=O)N3C[C@H](O)C[C@H]3C(=O)N[C@@H](C)c3ccc(-c4scnc4C)cc3)C(C)(C)C)n2)nnc1Nc1nc2cccnc2s1] |
| I-453 | 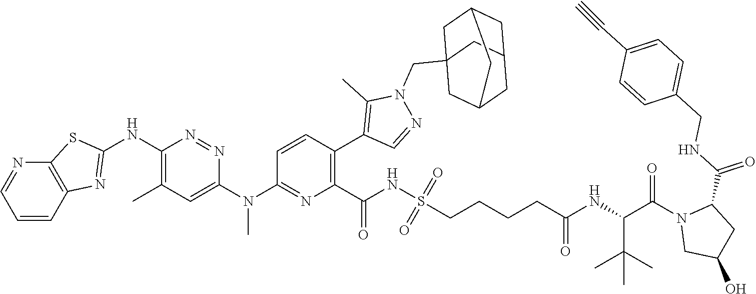 [SMILES: C#Cc1ccc(CNC(=O)[C@@H]2C[C@@H](O)CN2C(=O)[C@@H](NC(=O)CCCCS(=O)(=O)NC(=O)c2nc(N(C)c3cc(C)c(Nc4nc5cccnc5s4)nn3)ccc2-c2cnn(CC34CC5CC(CC(C5)C3)C4)c2C)C(C)(C)C)cc1] |
| I-454 | 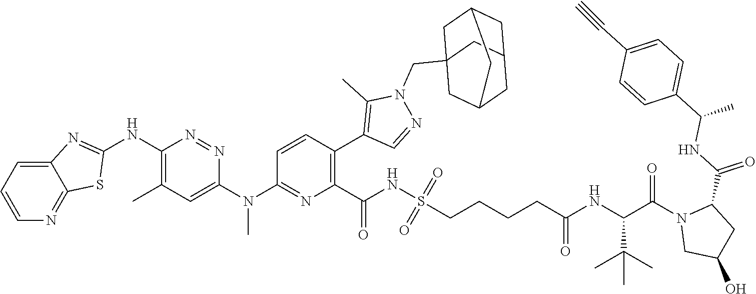 [SMILES: C#Cc1ccc([C@H](C)NC(=O)[C@@H]2C[C@@H](O)CN2C(=O)[C@@H](NC(=O)CCCCS(=O)(=O)NC(=O)c2nc(N(C)c3cc(C)c(Nc4nc5cccnc5s4)nn3)ccc2-c2cnn(CC34CC5CC(CC(C5)C3)C4)c2C)C(C)(C)C)cc1] |
| I-455 | 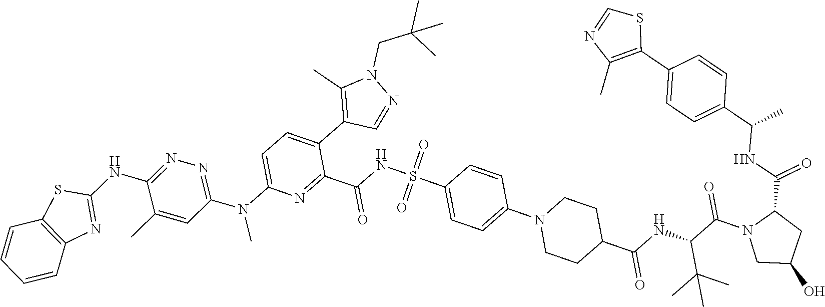 [SMILES: Cc1cc(N(C)c2ccc(-c3cnn(CC(C)(C)C)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2ccccc2s1] |
| I-456 | 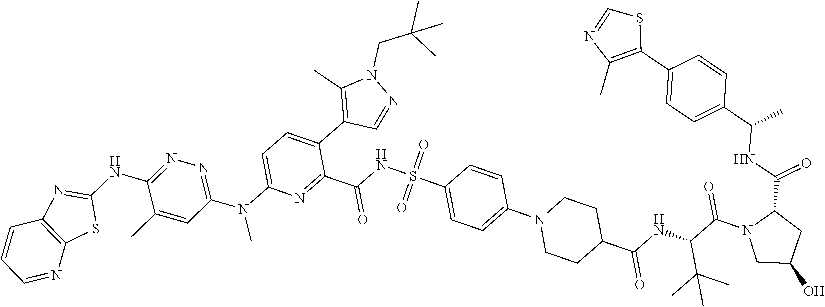 [SMILES: Cc1cc(N(C)c2ccc(-c3cnn(CC(C)(C)C)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2cccnc2s1] |
| I-457 | 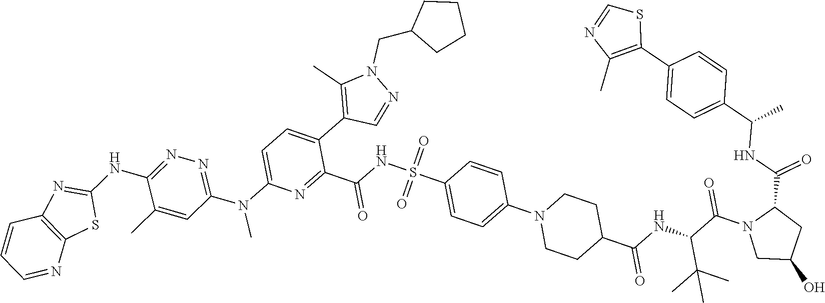 [SMILES: Cc1cc(N(C)c2ccc(-c3cnn(CC4CCCC4)c3C)c(C(=O)NS(=O)(=O)c3ccc(N4CCC(C(=O)N[C@H](C(=O)N5C[C@H](O)C[C@H]5C(=O)N[C@@H](C)c5ccc(-c6scnc6C)cc5)C(C)(C)C)CC4)cc3)n2)nnc1Nc1nc2cccnc2s1] |
| I-458 | 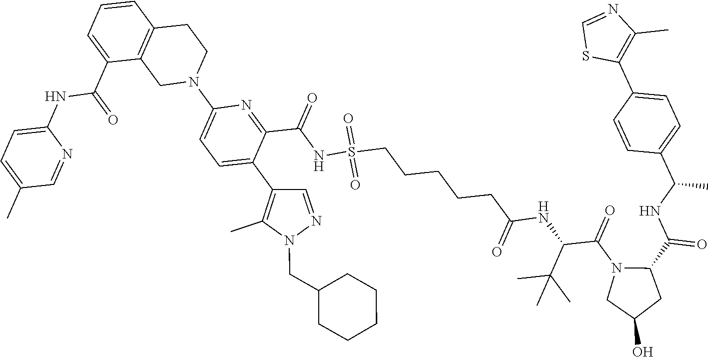 [SMILES: Cc1ccc(NC(=O)c2cccc3c2CN(c2ccc(-c4cnn(CC5CCCCC5)c4C)c(C(=O)NS(=O)(=O)CCCCCC(=O)N[C@H](C(=O)N4C[C@H](O)C[C@H]4C(=O)N[C@@H](C)c4ccc(-c5scnc5C)cc4)C(C)(C)C)n2)CC3)nc1] |
| I-459 | 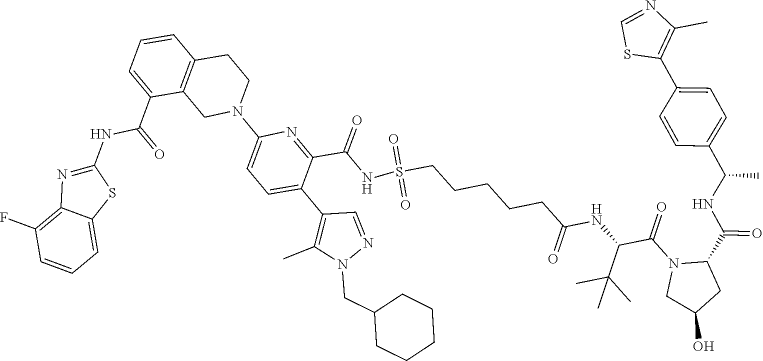 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6c(F)cccc6s5)c4C3)ccc2-c2cnn(CC3CCCCC3)c2C)C(C)(C)C)cc1] |
| I-460 | 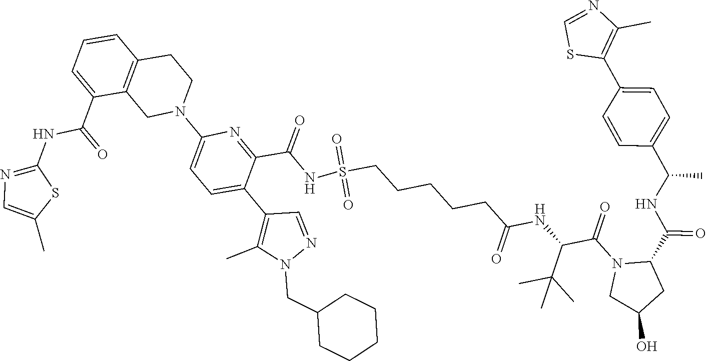 [SMILES: Cc1cnc(NC(=O)c2cccc3c2CN(c2ccc(-c4cnn(CC5CCCCC5)c4C)c(C(=O)NS(=O)(=O)CCCCCC(=O)N[C@H](C(=O)N4C[C@H](O)C[C@H]4C(=O)N[C@@H](C)c4ccc(-c5scnc5C)cc4)C(C)(C)C)n2)CC3)s1] |
| I-461 | 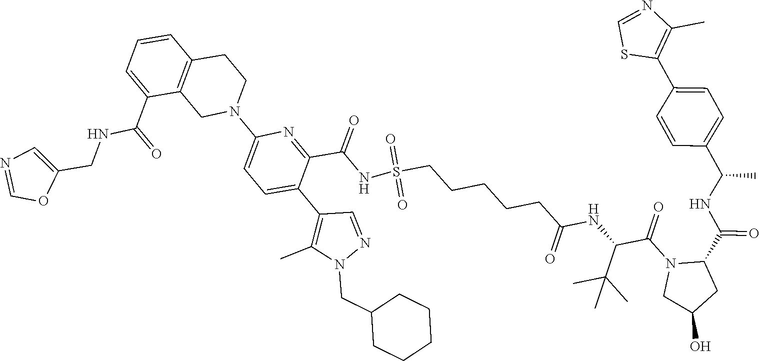 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)NCc5cnco5)c4C3)ccc2-c2cnn(CC3CCCCC3)c2C)C(C)(C)C)cc1] |
| I-462 | 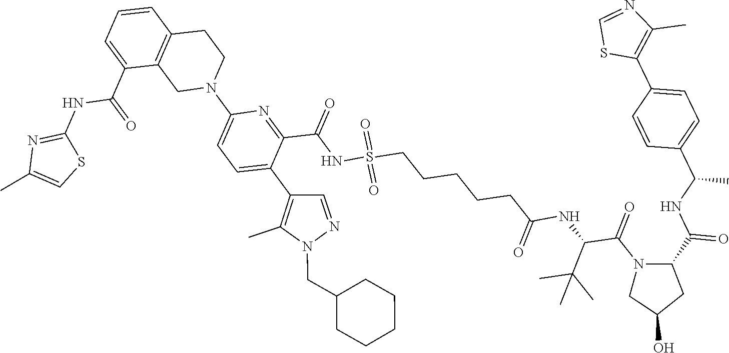 [SMILES: Cc1csc(NC(=O)c2cccc3c2CN(c2ccc(-c4cnn(CC5CCCCC5)c4C)c(C(=O)NS(=O)(=O)CCCCCC(=O)N[C@H](C(=O)N4C[C@H](O)C[C@H]4C(=O)N[C@@H](C)c4ccc(-c5scnc5C)cc4)C(C)(C)C)n2)CC3)n1] |
| I-463 | 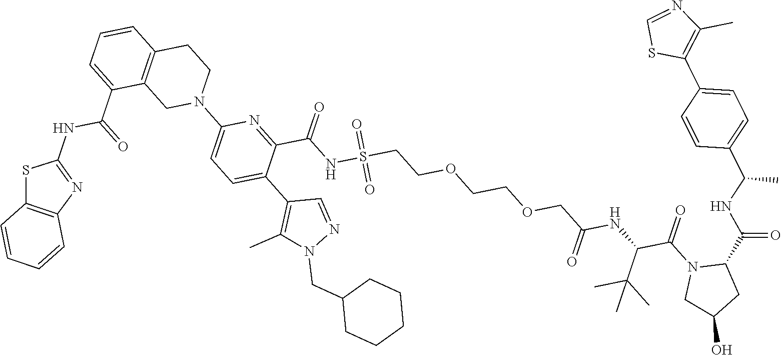 [SMILES: Cc1ncsc1-c1ccc([C@H](C)NC(=O)[C@@H]2C[C@@H](O)CN2C(=O)[C@@H](NC(=O)COCCOCCS(=O)(=O)NC(=O)c2nc(N3CCc4cccc(C(=O)Nc5nc6ccccc6s5)c4C3)ccc2-c2cnn(CC3CCCCC3)c2C)C(C)(C)C)cc1] |
| I-464 | 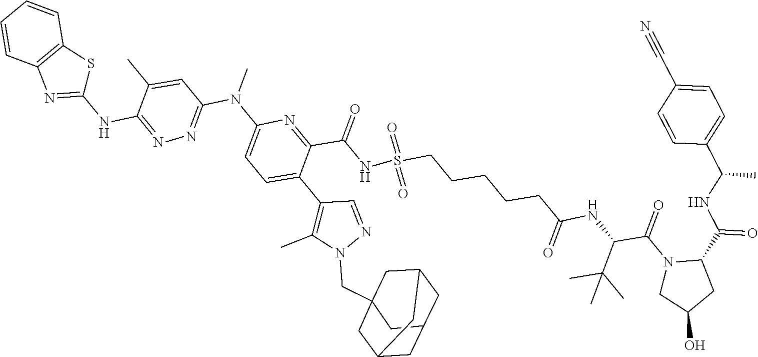 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)N[C@@H](C)c3ccc(C#N)cc3)C(C)(C)C)n2)nnc1Nc1nc2ccccc2s1] |
| I-465 | 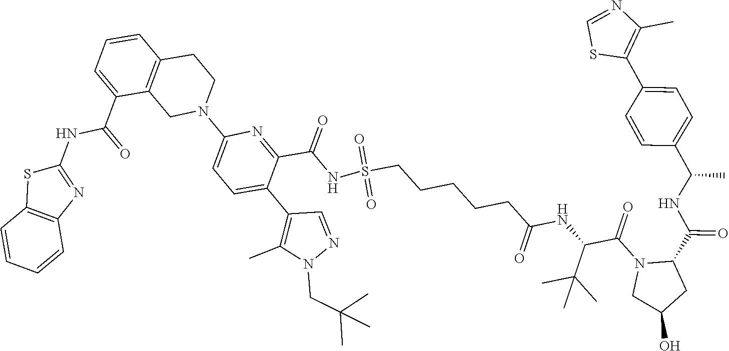 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC(C)(C)C)c2C)C(C)(C)C)cc1] |
| I-466 | 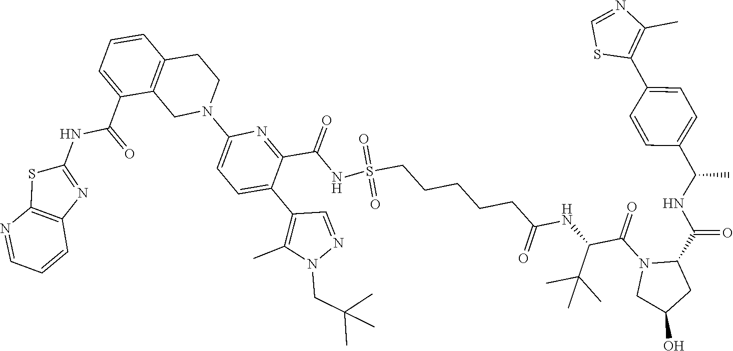 [SMILES: Cc1ncsc1-c1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC(C)(C)C)c2C)C(C)(C)C)cc1] |
| I-467 | 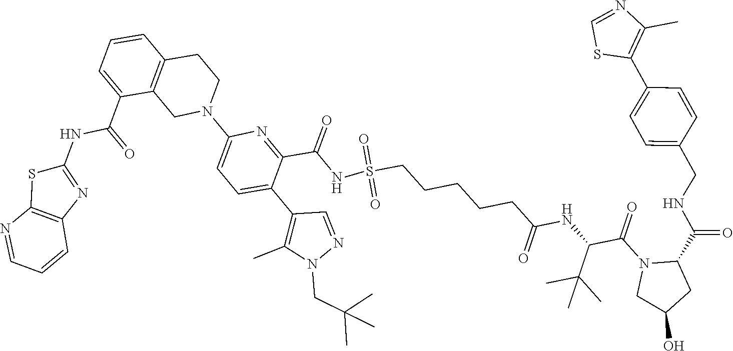 [SMILES: Cc1ncsc1-c1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC(C)(C)C)c2C)C(C)(C)C)cc1] |
| I-468 | 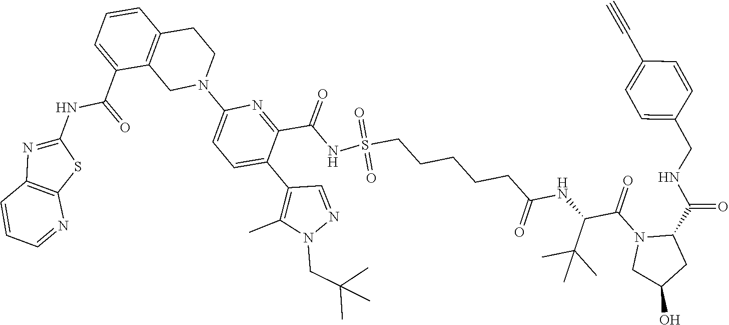 [SMILES: C#Cc1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC(C)(C)C)c2C)C(C)(C)C)cc1] |
| I-469 | 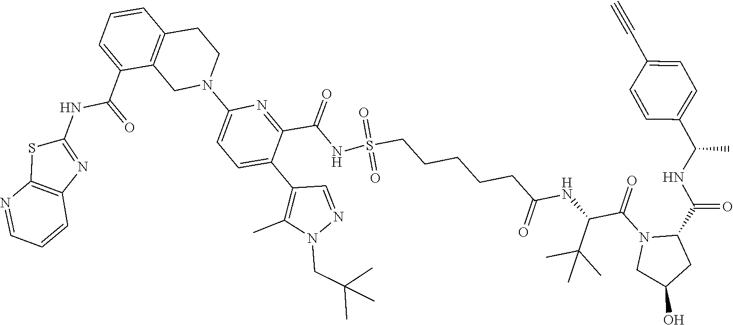 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC(C)(C)C)c2C)C(C)(C)C)cc1] |
| I-470 | 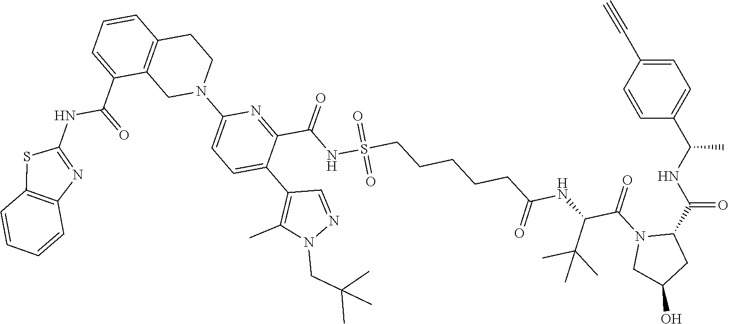 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC(C)(C)C)c2C)C(C)(C)C)cc1] |
| I-471 | 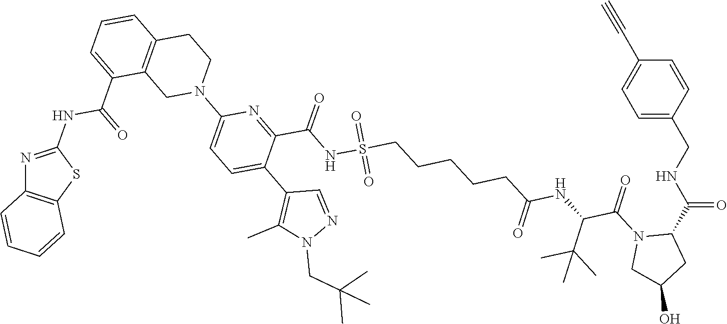 [SMILES: C#Cc1ccc(CNC(=O)[C@@H]2C[C@@H](O)CN2C(=O)[C@@H](NC(=O)CCCCCS(=O)(=O)NC(=O)c2nc(N3CCc4cccc(C(=O)Nc5nc6ccccc6s5)c4C3)ccc2-c2cnn(CC(C)(C)C)c2C)C(C)(C)C)cc1] |
| I-472 | 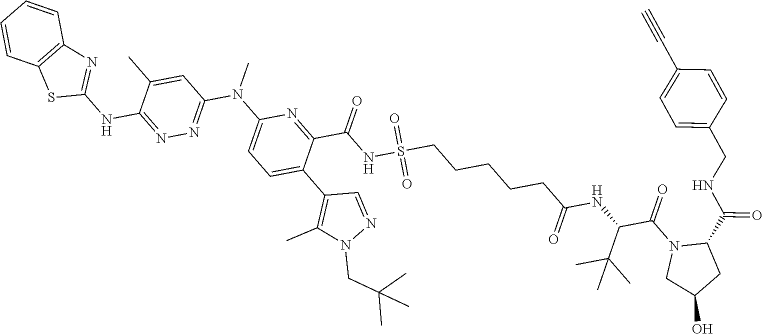 [SMILES: C#Cc1ccc(CNC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC(C)(C)C)c2C)C(C)(C)C)cc1] |
| I-473 | 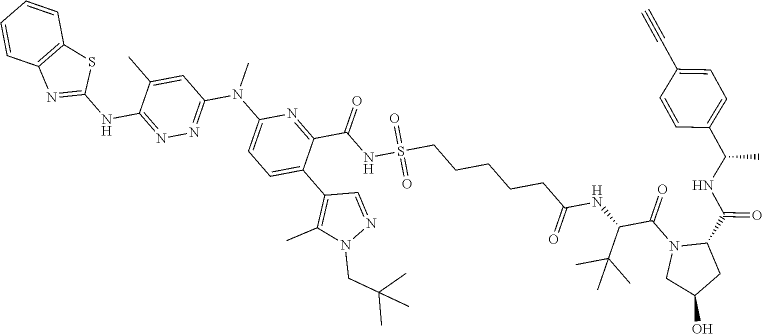 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC(C)(C)C)c2C)C(C)(C)C)cc1] |
| I-474 | 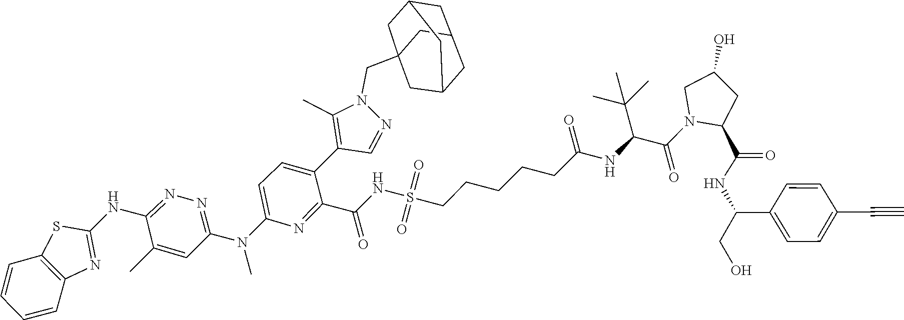 [SMILES: C#Cc1ccc([C@H](CO)NC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-475 | 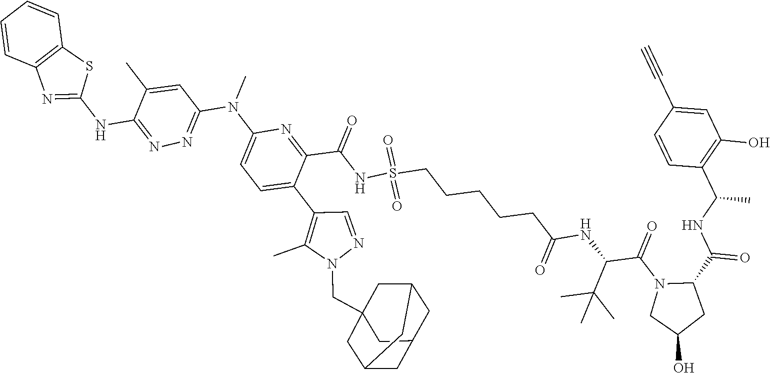 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)c(O)c1] |
| I-476 | 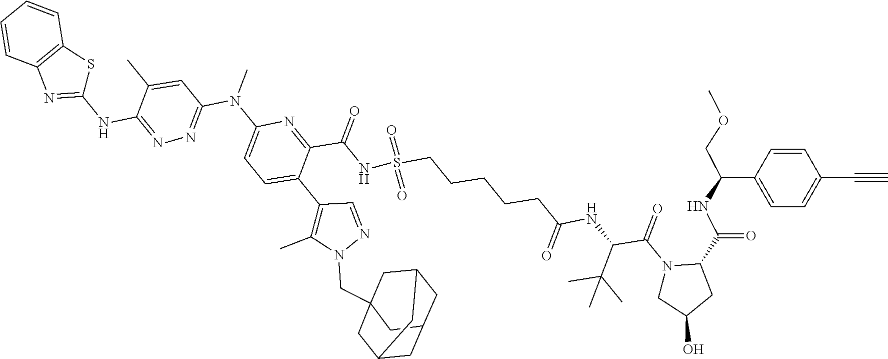 [SMILES: C#Cc1ccc([C@H](COC)NC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)cc1] |
| I-477 | 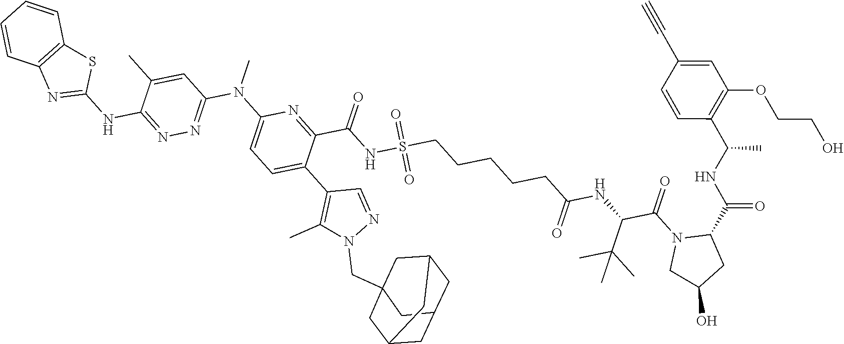 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N(C)c3cc(C)c(Nc4nc5ccccc5s4)nn3)ccc2-c2cnn(CC34CC5CC(CC(C5)C3)C4)c2C)C(C)(C)C)c(OCCO)c1] |
| I-478 | 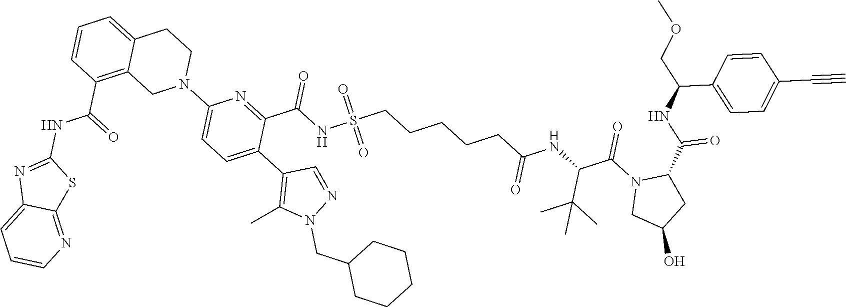 [SMILES: C#Cc1ccc([C@H](COC)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-479 | 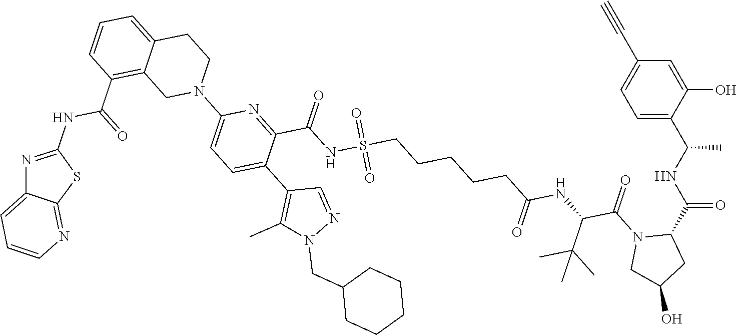 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)c(O)c1] |
| I-480 | 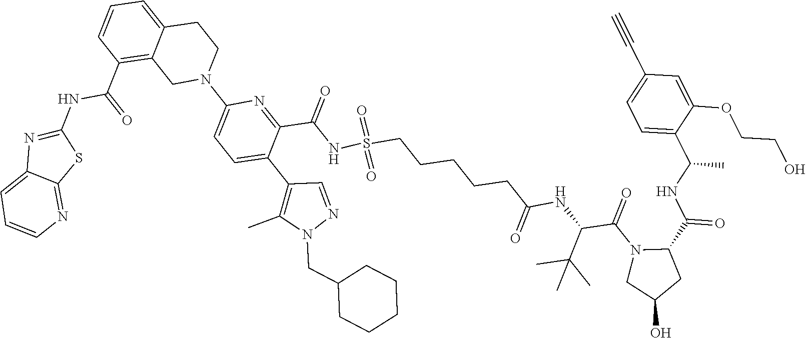 [SMILES: C#Cc1ccc([C@H](C)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)c(OCCO)c1] |
| I-481 | 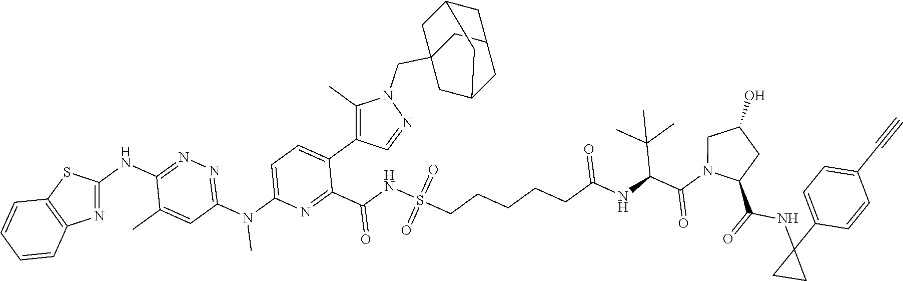 [SMILES: C#Cc1ccc(C2(NC(=O)[C@@H]3C[C@@H](O)CN3C(=O)[C@@H](NC(=O)CCCCCS(=O)(=O)NC(=O)c3nc(N(C)c4cc(C)c(Nc5nc6ccccc6s5)nn4)ccc3-c3cnn(CC45CC6CC(CC(C6)C4)C5)c3C)C(C)(C)C)CC2)cc1] |
| I-482 | 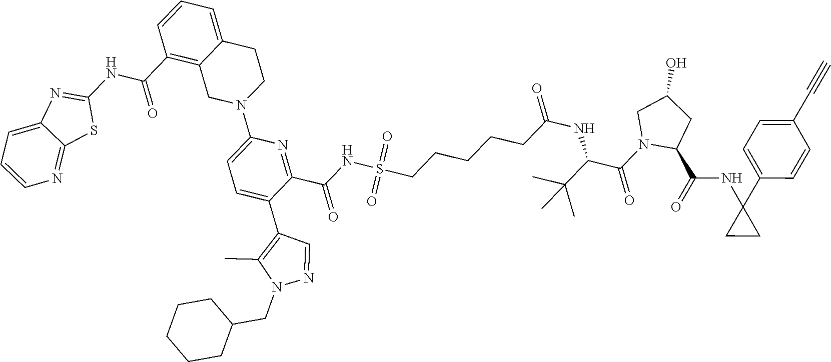 [SMILES: C#Cc1ccc(C2(NC(=O)[C@@H]3C[C@@H](O)CN3C(=O)[C@@H](NC(=O)CCCCCS(=O)(=O)NC(=O)c3nc(N4CCc5cccc(C(=O)Nc6nc7cccnc7s6)c5C4)ccc3-c3cnn(CC4CCCCC4)c3C)C(C)(C)C)CC2)cc1] |
| I-483 | 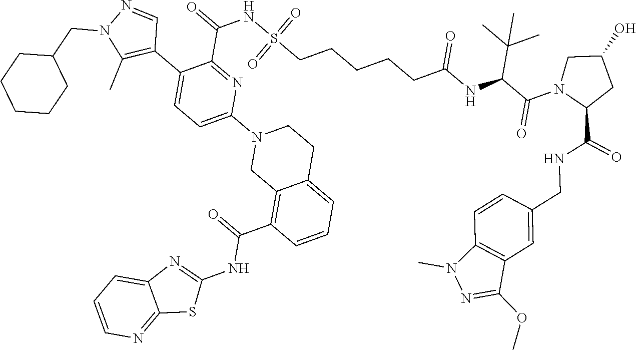 [SMILES: COc1nn(C)c2ccc(CNC(=O)[C@@H]3C[C@@H](O)CN3C(=O)[C@@H](NC(=O)CCCCCS(=O)(=O)NC(=O)c3nc(N4CCc5cccc(C(=O)Nc6nc7cccnc7s6)c5C4)ccc3-c3cnn(CC4CCCCC4)c3C)C(C)(C)C)cc12] |
| I-484 | 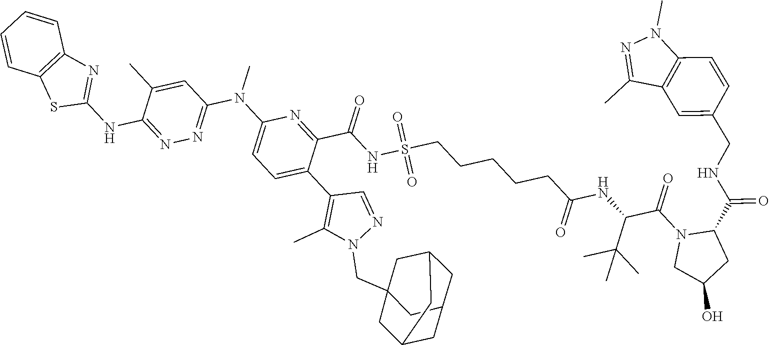 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)NCc3ccc4c(c3)c(C)nn4C)C(C)(C)C)n2)nnc1Nc1nc2ccccc2s1] |
| I-485 | 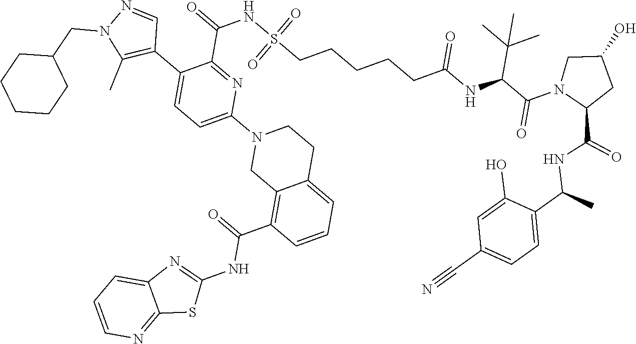 [SMILES: Cc1c(-c2ccc(N3CCc4cccc(C(=O)Nc5nc6cccnc6s5)c4C3)nc2C(=O)NS(=O)(=O)CCCCCC(=O)N[C@H](C(=O)N2C[C@H](O)C[C@H]2C(=O)N[C@@H](C)c2ccc(C#N)cc2O)C(C)(C)C)cnn1CC1CCCCC1] |
| I-486 | 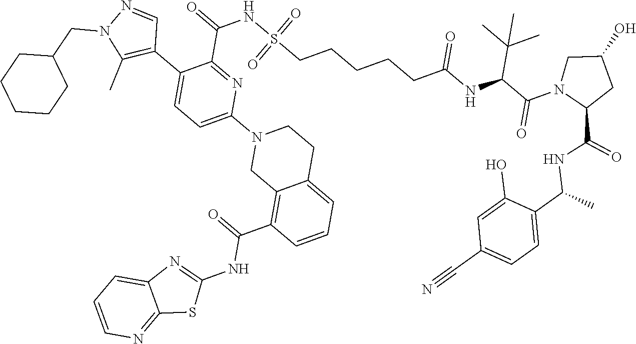 [SMILES: Cc1c(-c2ccc(N3CCc4cccc(C(=O)Nc5nc6cccnc6s5)c4C3)nc2C(=O)NS(=O)(=O)CCCCCC(=O)N[C@H](C(=O)N2C[C@H](O)C[C@H]2C(=O)N[C@H](C)c2ccc(C#N)cc2O)C(C)(C)C)cnn1CC1CCCCC1] |
| I-487 | 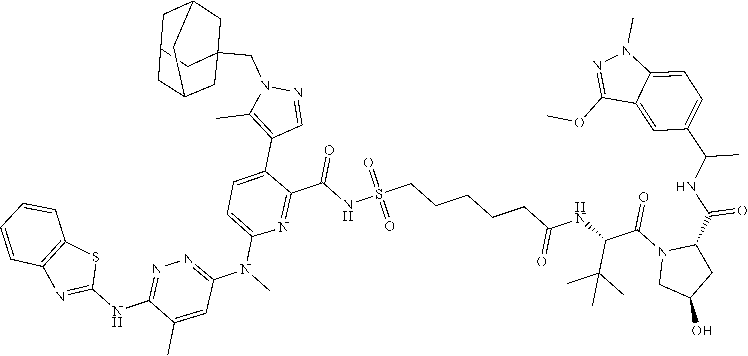 [SMILES: CCC1(Cn2ncc(-c3ccc(N(C)c4cc(C)c(Nc5nc6ccccc6s5)nn4)nc3C(=O)NS(=O)(=O)CCCCCC(=O)N[C@H](C(=O)N3C[C@H](O)C[C@H]3C(=O)NC(C)c3ccc4c(c3)c(OC)nn4C)C(C)(C)C)c2C)CC2CC(C)CC(C2)C1] |
| I-488 | 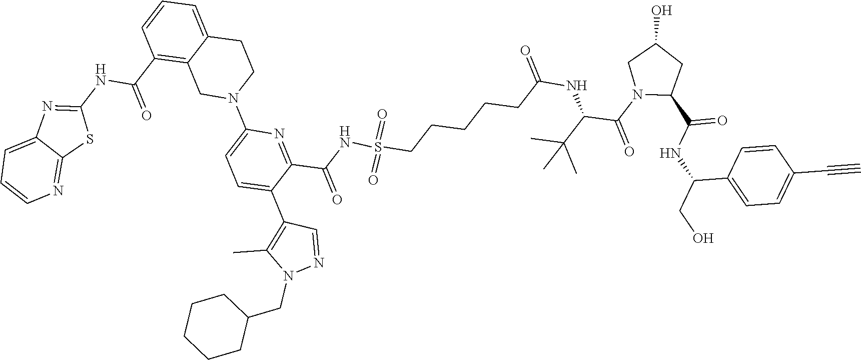 [SMILES: C#Cc1ccc([C@H](CO)NC(=O)[C@@H]2C[C@@H](O)CN2C(=O)[C@@H](NC(=O)CCCCCS(=O)(=O)NC(=O)c2nc(N3CCc4cccc(C(=O)Nc5nc6cccnc6s5)c4C3)ccc2-c2cnn(CC3CCCCC3)c2C)C(C)(C)C)cc1] |
| I-489 | 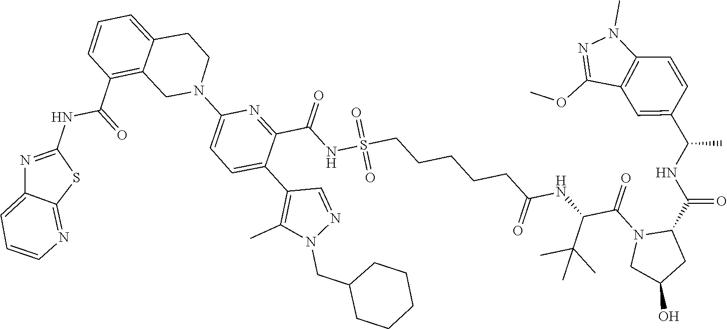 [SMILES: COc1nn(C)c2ccc([C@H](C)NC(=O)[C@@H]3C[C@@H](O)CN3C(=O)[C@@H](NC(=O)CCCCCS(=O)(=O)NC(=O)c3nc(N4CCc5cccc(C(=O)Nc6nc7cccnc7s6)c5C4)ccc3-c3cnn(CC4CCCCC4)c3C)C(C)(C)C)cc12] |
| I-490 | 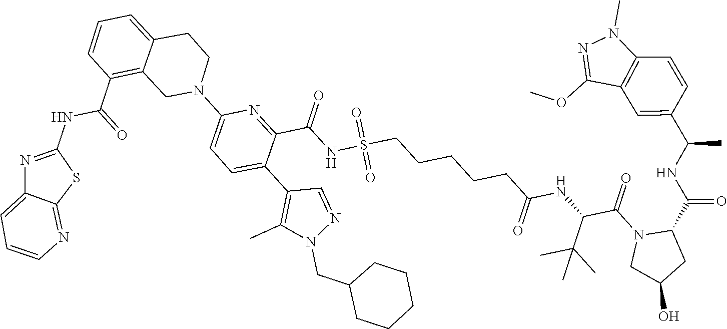 [SMILES: COc1nn(C)c2ccc([C@@H](C)NC(=O)[C@@H]3C[C@@H](O)CN3C(=O)[C@@H](NC(=O)CCCCCS(=O)(=O)NC(=O)c3nc(N4CCc5cccc(C(=O)Nc6nc7cccnc7s6)c5C4)ccc3-c3cnn(CC4CCCCC4)c3C)C(C)(C)C)cc12] |
| I-491 | 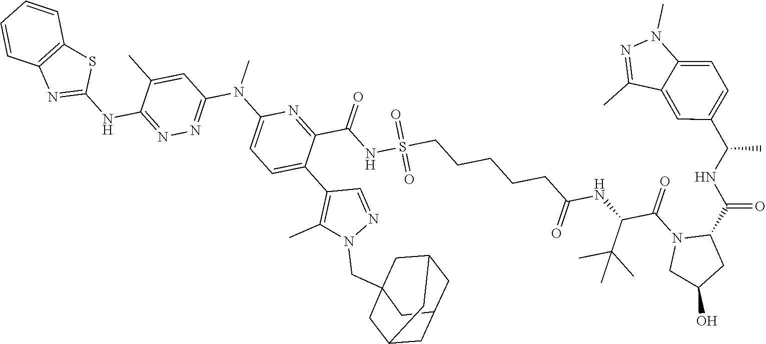 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)N[C@@H](C)c3ccc4c(c3)c(C)nn4C)C(C)(C)C)n2)nnc1Nc1nc2ccccc2s1] |
| I-492 | 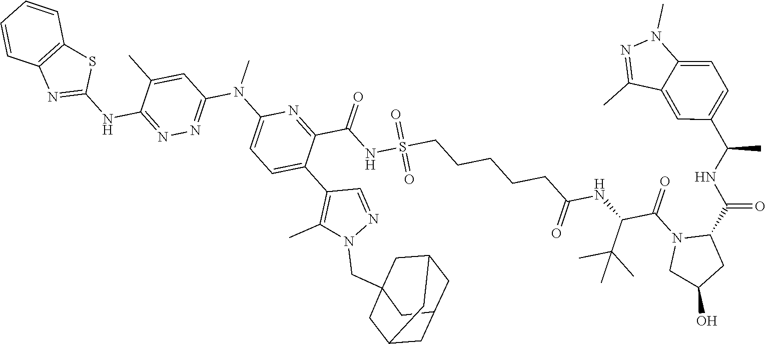 [SMILES: Cc1cc(N(C)c2ccc(-c3cnn(CC45CC6CC(CC(C6)C4)C5)c3C)c(C(=O)NS(=O)(=O)CCCCCC(=O)N[C@H](C(=O)N3C[C@H](O)C[C@H]3C(=O)N[C@H](C)c3ccc4c(c3)c(C)nn4C)C(C)(C)C)n2)nnc1Nc1nc2ccccc2s1] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio) pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl) ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3rd edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below; where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In certain embodiments, compounds of the present invention are generally prepared according to Scheme 1 set forth below:

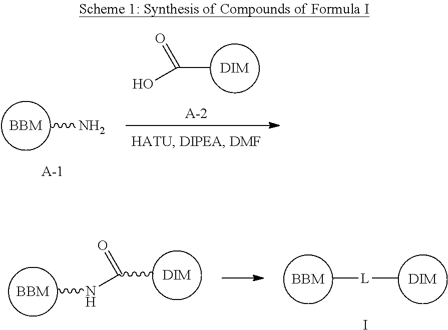

As depicted in Scheme 1, above, amine A-1 is coupled to acid A-2 using the coupling agent HATU in the presence of the base DIPEA in DMF to form a compound of formula I with a linker comprising an amide bond. The squiggly bond, [symbol] , represents the portion of the linker between BBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 2 set forth below:

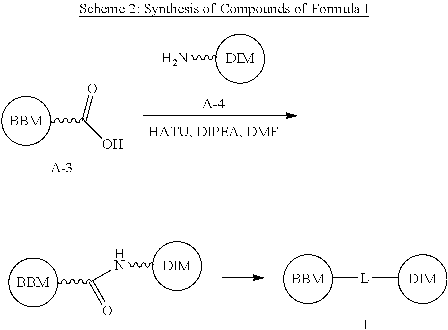

As depicted in Scheme 2, above, acid A-3 is coupled to amine A-4 using the coupling agent HATU in the presence of the base DIPEA in DMF to form a compound of formula I with a linker comprising an amide bond. The squiggly bond, [symbol] , represents the portion of the linker between BBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 3 set forth below:

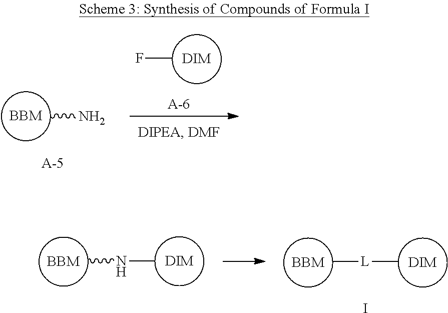

As depicted in Scheme 3, above, an S_{N}Ar displacement of fluoride A-6 by amine A-5 is effected in the presence of the base DIPEA in DMF to form a compound of formula I with a linker comprising a secondary amine. The squiggly bond, [symbol] , represents the portion of the linker between BBM and the terminal amino group of A-5.

In certain embodiments, compounds of the present invention are generally prepared according to Scheme 4 set forth below:

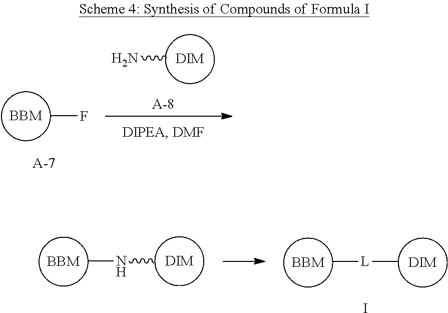

As depicted in Scheme 4, above, an S_{N}Ar displacement of fluoride A-7 by amine A-8 is effected in the presence of the base DIPEA in DMF to form a compound of formula I with a linker comprising a secondary amine. The squiggly bond, [symbol] , represents the portion of the linker between DIM and the terminal amino group of A-8.

In certain embodiments, compounds of the present invention are generally prepared according to Scheme 5 set forth below:

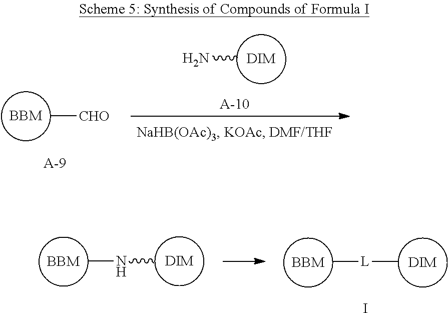

As depicted in Scheme 5, above, reductive amination of the mixture of aldehyde A-9 and amine A-10 is effected in the presence of NaHB(OAc)₃ and KOAc in DMF/THF to form a compound of formula I with a linker comprising a secondary amine. The squiggly bond, [symbol] , represents the portion of the linker between DIM and the terminal amino group of A-8.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B, and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit a BCL-XL protein, or a mutant thereof, in a biological sample or in a patient. In certain embodiments, the amount of compound in compositions of this invention is such that is effective to measurably degrade and/or inhibit an BCL-XL protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of a BCL-XL protein, or a mutant thereof.

As used herein, the term “degratorily active metabolite or residue thereof” means that a metabolite or residue thereof is also a degrader of an BCL-XL protein, or a mutant thereof.

Compositions of the present invention may be administered orally; parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation and/or inhibition of BCL-XL protein activity.

As used herein, the terms “BCL-XL-mediated” disorders, diseases, and/or conditions as used herein means any disease or other deleterious condition in which one or more BCL-XL, or a mutant thereof, are known to play a role. Accordingly, another embodiment of the present invention relates to treating or lessening the severity of one or more diseases in which BCL-XL, or a mutant thereof, are known to play a role. For example in some embodiments, the BCL-XL-mediated disorders, diseases, and/or conditions is cancer, autoimmune disease, or inflammation.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

The term “biological sample”, as used herein, includes, without limitation, cell cultures or extracts thereof: biopsied material obtained from a mammal or extracts thereof; and blood, saliva, urine, feces, semen, tears, or other body fluids or extracts thereof.

In some embodiments, the present invention provides a method of degrading BCL-XL protein or a mutant thereof in a biological sample. The method comprises contacting the biological sample with a therapeutically effective amount of a provided compound or a therapeutically acceptable salt thereof.

Inhibition and/or degradation of a BCL-XL protein, or a protein selected from BCL-XL, or a mutant thereof, activity in a biological sample is useful for a variety of purposes that are known to one of skill in the art. Examples of such purposes include, but are not limited to, blood transfusion, organ-transplantation, biological specimen storage, and biological assays.

In some embodiments, the present invention provides a method of degrading BCL-XL protein or a mutant thereof in a patient in need thereof. The method comprises administering to the patient in need thereof a therapeutically effective amount of a provided compound or a therapeutically acceptable salt thereof.

In some embodiments, the present invention provides a method of degrading other BCL-2 family proteins (e.g., BCL-2, BCL-W, and/or MCL-1) or a mutant thereof in a patient in need thereof. The method comprises administering to the patient in need thereof a therapeutically effective amount of a provided compound or a therapeutically acceptable salt thereof. Accordingly without limitation, the present compounds may also be useful for treating BCL-2-mediated, BCL-W-mediated, or MCL-1-mediated disorders, diseases, and/or conditions.

In some embodiments, the present invention provides compounds that have pro-apoptotic properties. The ability to reactivate the apoptotic process in cells is of major therapeutic interest in the treatment of cancer, immune diseases, autoimmune diseases. In particular, the compounds according to the present invention are useful in the treatment of chemoresistant or radioresistant cancers. In another embodiment, the compounds of the invention may be used for treating diseases or conditions characterized by an excess or a deregulated activity of platelets, especially pro-thrombotic conditions.

In some embodiments, the present invention provides a method of killing one or more cancers in a patient in need thereof. The method comprises administering to the patient in need thereof a therapeutically effective amount of a provided compound or a therapeutically acceptable salt thereof. In some embodiments, the method of killing one or more cancers in a patient in need thereof comprises degradation of BCL-XL or a mutant thereof.

In some embodiments, the invention provides a method of treating cancer in a patient in need thereof comprising administering to the patient a therapeutically acceptable amount of a provided compound or a therapeutically acceptable salt thereof.

In some embodiments, the invention provides a method of selectively killing one or more cancer cells in a sample, the method comprising contacting the sample with an effective amount of a provided compound or a therapeutically acceptable salt thereof, or a pharmaceutically acceptable composition thereof. In another aspect, the present invention encompasses a method of selectively killing one or more cancer cells in a patient in need thereof, the method comprising administering to the patient in need thereof a provided compound or a therapeutically acceptable salt thereof, or a pharmaceutically acceptable composition thereof.

By selectively killing one or more cancer cells is meant a composition of the invention docs not appreciably kill non-cancer cells at the same concentration. In some embodiments, a provided compound or a therapeutically acceptable salt thereof has reduced platelet toxicity and retained or improved toxicity in cancer cells when compared to BCL-XL inhibitors.

In some embodiments, the method comprises killing one or more cancer cells in a patient with cancer. As such, the methods of the disclosure may be used to treat a tumor derived from a neoplasm or a cancer. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma. AIDS-related cancers. AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, cpendymoma, medulloblastoma, supratentorial primitive neurocctodermal tumors, visual pathway and hypothalamic gliomas, breast cancer, bronchial adenomas/carcinoids. Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, choriocarcinoma, chronic lymphocytic leukemia, chronic myelogenous leukemia, chronic myeloproliferative disorders, colon cancer, cutaneous T-cell lymphoma, desmoplastic small round cell tumor, endometrial cancer, cpendymoma, esophageal cancer. Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, glioblastoma, gliomas (adult, childhood brain stem, childhood cerebral astrocytoma, childhood visual pathway and hypothalamic), gastric carcinoid, hairy cell leukemia, head and neck cancer, hepatocellular (liver) cancer. Hodgkin lymphoma, hypopharyngeal cancer, hypothalamic and visual pathway glioma (childhood), intraocular melanoma, islet cell carcinoma. Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related. Burkitt, cutaneous T-cell. Hodgkin, non-Hodgkin, primary central nervous system), macroglobulinemia (Waldenström), malignant fibrous histiocytoma of bone/osteosarcoma, medulloblastoma (childhood), melanoma, intraocular melanoma. Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, mycloproliferative disorders (chronic), nasal cavity and paranasal sinus cancer, nasopharyngeal carcinoma, neuroblastoma, non-Hodgkin lymphoma, non-small cell renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In certain embodiments, a cancer is selected from synovial sarcoma, Burkitt lymphoma, Hodgkin lymphoma, multiple myeloma, neuroblastoma, glioblastoma, small cell lung cancer, pancreatic cancer, hepatocellular (liver) cancer, endometrial cancer, ovarian cancer, cervical cancer, breast cancer, prostate cancer, bladder cancer, melanoma, rhabdomyosarcoma, osteosarcoma/malignant fibrous histiocytoma of bone, choriocarcinoma, kidney cancer (renal cell cancer), thyroid cancer, and leukemias (acute lymphoblastic, acute myeloid, chronic lymphocytic, and chronic myelogenous).

In some embodiments, the BCL-XL-mediated disorders, diseases, and/or conditions is an autoimmune disease. In some embodiments, the present invention provides a method of treating an autoimmune disease in a patient in need thereof, comprising administering to the patient a provided compound or a therapeutically acceptable salt thereof.

In some embodiments, the autoimmune disease is rheumatoid arthritis, psoriatic arthritis, juvenile idiopathic arthritis, multiple sclerosis, systemic lupus erythematosus, myasthenia gravis, juvenile onset diabetes, glomerulonephritis, autoimmune thyroiditis, Behcet's disease, Crohn's disease, ulcerative colitis, bullous pemphigoid, sarcoidosis, psoriasis, ichthyosis, Graves ophthalmopathy, psoriasis, psoriasis inflammatory bowel disease, or asthma.

In some embodiments, the BCL-XL-mediated disorders, diseases, and/or conditions is inflammation. In some embodiments, the present invention provides a method of treating inflammation in a patient in need thereof, comprising administering to the patient a provided compound or a therapeutically acceptable salt thereof.

In some embodiments, the inflammation is an inflammatory condition associated with lupus erythmatosus, psoriasis, psoriatic arthritis, lupus nephritis, rheumatoid arthritis, multiple sclerosis, ulcerative colitis, myasthenia gravis, immune thrombocytopenia purpura (ITP), thrombotic thrombocytopenia purpura (TTP), Grave's disease, Hashimoto's thyroiditis, Crohn's disease, autoimmune hemolytic anemias, insulin dependent diabetes mellitus, glomerulonephritis, rheumatic fever, osteoarthritis, gouty arthritis, dermatitis, bronchitis, rhinitis, asthma, Sjogrens' syndrome, meningitis, adrenoleukodystrophy, CNS vasculitis, mitochondrial myopathies, Amyotrophic Lateral Sclerosis, or Alzheimer's disease.

As discussed in more detail herein, some embodiments provide methods for treating and/or prevention of various inflammatory disorders, diseases and conditions. Such inflammatory disorders, diseases and conditions include, without limitation, systemic autoimmune diseases such as, for example, lupus erythematosus, rheumatoid arthritis, multiple sclerosis, and psoriasis; and organ specific autoimmune diseases such as, for example, ulcerative colitis, myasthenia gravis, Grave's disease, Hashimoto's thyroiditis, Crohn's disease, lupus nephritis, autoimmune hemolytic anemias, ITP, TTP, insulin dependent diabetes mellitus, glomerulonephritis, and rheumatic fever. Other inflammatory diseases that may be treated in accordance with this disclosure include, without limitation, other inflammatory arthritic conditions such as psoriatic arthritis, osteoarthritis and gouty arthritis, as well as other inflammatory conditions such as conjunctivitis, dermatitis, bronchitis, rhinitis etc., brought about by injury, allergies, infections, microorganisms, trauma, or physical or chemical agents. The treatment of inflammatory aspects of asthma, Sjogrens' syndrome, meningitis, adrenoleukodystrophy, CNS vasculitis, mitochondrial myopathies, Amyotrophic Lateral Sclerosis, Alzheimer's disease, or tumors is also contemplated as part of this disclosure. Examples of mitochondrial myopathies include MELAS syndrome, MERF syndrome, Leber's disease, Wernicke's encephalopathy: Rett syndrome, homocystinuria, hyperprolinemia, nonketotic hyperglycinemia, hydroxybutyric aminoaciduria, sulfite oxidase deficiency, and combined systems disease (B 12 deficiency). In association with such prevention and/or treatment, articles of manufacture, compositions, methods of use, and medical treatments by the compounds described herein are also provided.

In some embodiments, a provided compound is an anti-senolytic agent. Accordingly in some embodiments, the present invention provides a method of treating an age-related disease including various fibrotic diseases, osteoarthritis, and kidney diseases. In some embodiments, the age-related disease is diabetes, obesity; cardiac dysfunction, vascular hyporeactivity/calcification, AV fistulae, frailty, age-related muscle loss (sarcopenia), chemotherapy complication, radiation complication, cancer, bone marrow transplant complication, organ transplantation complication, myeloma/monoclonal gammopathy of undetermined significance (MGUS), age-related cognitive dysfunction, Alzheimer's disease, Parkinson's disease, or amyotrophic lateral sclerosis. See e.g., Kirkland and Tchkonia. “Senolytic drugs: From discovery to translation.” Journal of Internal Medicine 2020, 288(5):518-536.

It is believed that a provided compound or a pharmaceutically acceptable salt thereof may possess satisfactory pharmacological profile and promising biopharmaceutical properties, such as toxicological profile, metabolism and pharmacokinetic properties, solubility; and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the invention are useful in preventing or reducing the risk of developing any of the diseases referred to herein: e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation; treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 R^{A}, azathioprinc, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively; those agents may be part of a single dosage form, mixed together with a compound of this invention in a single composition. If administered as part of a multiple dosage regime, the two active agents may be submitted simultaneously; sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes. 10 minutes. 15 minutes. 30 minutes. 1 hour. 2 hours. 3 hours. 4 hours. 5, hours. 6 hours. 7 hours. 8 hours. 9 hours. 10 hours. 11 hours. 12 hours. 13 hours. 14 hours. 15 hours. 16 hours. 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®). D-penicillamine (Depen® or Cuprimine®), azathioprinc (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®). “anti-IL-1” agents such as anakinra (Kineret®) and rilonacept (Arcalyst®), canakinumab (Ilaris®), anti-Jak inhibitors such as tofacitinib, antibodies such as rituximab (Rituxan®). “anti-T-cell” agents such as abatacept (Orencia®). “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquacmin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent® Qvar®, and Vanceril®), triamcinolone acetonide (Azmacort®), mometasone (Asthmanex®), budesonide (Pulmocort®), and flunisolide (Acrobid®). Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline. IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®). D-penicillamine (Depen® or Cuprimine®), azathioprinc (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®). “anti-IL-1” agents such as anakinra (Kineret®) and rilonacept (Arcalyst®), antibodies such as rituximab (Rituxan®). “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanczumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprinc (Imuran®) and anticoagulants such as heparin (Calcinparine® or Liquacmin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidinc®), antidiarrheals such as diphenoxylate (Lomotil®) and loperamide (Imodium®), bile acid binding agents such as cholestyramine, alosctron (Lotroncx®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin®, HFA, Proventil®, HFA), levalbuterol (Xopencx®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Acrobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin®, HFA, Proventil®, HFA), levalbuterol (Xopencx®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanccril®), triamcinolone acetonide (Azmacort®), mometasone (Asthmanex®), budesonide (Pulmocort®), flunisolide (Acrobid®), Afviar®, Symbicort®, and Dulera®.

In some embodiments, the present invention provides a method of treating HIV comprising administering to a patient in need thereof a provided compound and one or more additional therapeutic agents selected from nucleoside reverse transcriptase inhibitors such as zidovudine (Retrovirk), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzcon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo R, Sun Pharmaceuticals); and vismodegib (Erivedge R, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparzak, AstraZeneca); rucaparib (Rubracak, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance R Pfizer): ribociclib (Kisqali®, Novartis): abemaciclib (Ly2835219, Eli Lilly); and trilaciclib (GIT28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo R, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene: NCT02677922): AG221 (Celgene, NCT02677922: NCT02577406): BAY1436032 (Bayer, NCT02746081): IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerrak, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech) and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, the present invention provides a method of treating cancer (e.g., leukemias, lymphomas, etc.) comprising administering to a patient in need thereof a provided compound and one or more BCL-2 inhibitors.

In some embodiments, one or more other therapeutic agent is a MCL-1 inhibitor. MCL-1 inhibitors which may be used in the present invention include marinopyrole A (Maritoclax), VU661013, AZD5991, S63845, S64315 (MIK665), A-1210477, and UMI-77.

In some embodiments, the present invention provides a method of treating cancer (e.g., leukemias, lymphomas, etc.) comprising administering to a patient in need thereof a provided compound and one or more MCL-1 inhibitors.

In some embodiments, one or more other therapeutic agent is an androgen receptor inhibitor. Approved androgen receptor inhibitors useful in the present invention include enzalutamide (Xtandi®, Astellas/Medivation): approved inhibitors of androgen synthesis include abiraterone (Zytiga®, Centocor/Ortho): approved antagonist of gonadotropin-releasing hormone (GnRH) receptor (degaralix, Firmagon R, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista R, Eli Lilly).

In some embodiments, one or more other therapeutic agent is an inhibitor of bone resorption. An approved therapeutic which inhibits bone resorption is Denosumab (Xgeva R, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa R, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972: NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008: Sanofi-Genzyme), which is being studied for melanoma (NCT00923169): renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE, gpNMB is a protein overexpressed by multiple tumor types associated with cancer cells ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies: compounds which target, decrease or inhibit the activity of Flt-3: Hsp90 inhibitors such as 17-AAG (17-allylaminogeldanamycin. NSC330507). 17-DMAG (17-dimethylaminoethylamino-17-demethoxy-geldanamycin. NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as selumctinib (Kosclugo®) from Array BioPharma, AZd6244 from AstraZeneca. PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly: Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine. Vidaza®, Celgene): omacetaxine mepesuccinate (cephalotaxine ester) (protein synthesis inhibitor, Synribo®; Teva Pharmaceuticals); asparaginase Erwinia chrysanthemi (enzyme for depletion of asparagine·Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis): capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include; bevacizumab (Avastin R. Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi); VEGFR inhibitors, such as regorafenib (Stivarga®, Bayer), vandetanib (Caprelsa®, AstraZeneca), axitinib (Inlyta®, Pfizer), and lenvatinib (Lenvima®, Eisai); Raf inhibitors, such as sorafenib (Nexavar®, Bayer AG and Onyx), dabrafenib (Tafinlar®, Novartis), and vemurafenib (Zelboraf®, Genentech/Roche); MEK inhibitors, such as selumetinib (Koselugo®, AstraZeneca), binimetinib (Mcktovi®, Array BioPharma), cobimetanib (Cotellic®, Exelexis/Genentech/Roche), and trametinib (Mekinist®, Novartis): Bcr-Abl tyrosine kinase inhibitors, such as imatinib (Gleevec®, Novartis), nilotinib (Tasigna®, Novartis), dasatinib (Sprycel®, BristolMyersSquibb), bosutinib (Bosulif®, Pfizer), and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Bochringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as lenvatinib (Lenvima®, Easai), sunitinib (Sutent®, Pfizer), pontatinib (Iclusig®, Ariad), and pazopanib (Votrient®, Novartis); ALK inhibitors such as crizotinib (Xalkori®, Pfizer), ceritinib (Zykadia®, Novartis), brigatinib (Alunbrig®, Ariad), lorlatinib (Lorbrena®, Pfizer), and alectinib (Alecenza®, Genentech/Roche): Bruton's tyrosine kinase inhibitors, such as ibrutinib (Imbruvica®, Pharmacyclics/Janssen); Flt3 receptor inhibitors, such as midostaurin (Rydapt®, Novartis) and gilteritinib (Xospata®, Astellas); FGFR inhibitors such as pemigatinib (Pemazyre®, Incyte), nintedanib (Ofev®, Boehringer Ingelheim), and erdafitinib (Balversa®, Janssen Pharmaceuticals); RET inhibitors such as selpercatinib (Retivmo®, Eli Lilly), pralsetinib (Gavreto®, Blueprint Medicines); KIT inhibitors such as ripretinib (Qinlock®, Deciphera Pharmaceuticals), and avapritinib (Ayvakit®, Blueprint Medicines).

In some embodiments, the present invention provides a method of treating cancer comprising administering to a patient in need thereof a provided compound and one or more additional therapeutic agents selected from tyrosine kinase inhibitors or RAS-RAF-MEK-ERK pathway inhibitors.

In some embodiments, the present invention provides a method of treating cancer comprising administering to a patient in need thereof a provided compound and one or more FGFR inhibitors.

In some embodiments, the present invention provides a method of treating cancer comprising administering to a patient in need thereof a provided compound and one or more RET inhibitors.

In some embodiments, the present invention provides a method of treating cancer comprising administering to a patient in need thereof a provided compound and one or more KIT inhibitors.

In some embodiments, the present invention provides a method of treating cancer comprising administering to a patient in need thereof a provided compound and one or more ALK inhibitors.

In some embodiments, the present invention provides a method of treating cancer comprising administering to a patient in need thereof a provided compound and one or more RAS inhibitors.

In some embodiments, the present invention provides a method of treating cancer comprising administering to a patient in need thereof a provided compound and one or more MEK inhibitors.

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals. S. Korea); ruxolitinib (Jakafi®, Incyte Corporation); PTC299 (PTC Therapeutics); CP-547.632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs, host disease comprising administering to a patient in need thereof a provided compound and one or more additional therapeutic agents selected from a steroid, cyclosporin. FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenia purpura (ITP), rheumatoid arthritis, psoriatic arthritis, osteoarthritis, Still's disease, juvenile arthritis, diabetes, myasthenia gravis. Hashimoto's thyroiditis, Ord's thyroiditis, Graves disease, autoimmune thyroiditis, Sjogren's syndrome, multiple sclerosis, systemic sclerosis. Lyme neuroborreliosis. Guillain-Barre syndrome, acute disseminated encephalomyelitis. Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia. Wegener's granulomatosis, psoriasis, alopecia universalis, Behcet's disease, chronic fatigue, dysautonomia, membranous glomerulonephropathy; endometriosis, interstitial cystitis, pemphigus vulgaris, bullous pemphigoid, neuromyotonia, scleroderma, vulvodynia, a hyperproliferative disease, rejection of transplanted organs or tissues. Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity: allergic conjunctivitis, allergic rhinitis, and atopic dermatitis, asthma, appendicitis, atopic dermatitis, asthma, allergy: blepharitis, bronchiolitis, bronchitis, bursitis, cervicitis, cholangitis, cholecystitis, chronic graft rejection, colitis, conjunctivitis. Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy; interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia. B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease). Behcet's disease. Sjogren's syndrome, systemic sclerosis, osteoporosis, bone cancer, bone metastasis, a thromboembolic disorder, (e.g., myocardial infarct, angina pectoris, reocclusion after angioplasty; restenosis after angioplasty: reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenia states. Goodpasture's syndrome, atherosclerosis. Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenia purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves disease.

In another embodiment, the present invention provides a method of treating or lessening the severity of a disease comprising administering to a patient in need thereof a provided compound and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome. Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary: airways or lung disease (COPD, COAD or COLD), including chronic bronchitis or dyspnea associated therewith, emphysema, as well as exacerbation of airways hyperreactivity consequent to other drug therapy, in particular other inhaled drug therapy: bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis. Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology; including autoimmune hematological disorders (e.g. hemolytic anemia, aplastic anemia, pure red cell anemia and idiopathic thrombocytopenia), systemic lupus crythematosus, rheumatoid arthritis, polychondritis, sclerodoma, Wegener granulamatosis, dermatomyositis, chronic active hepatitis, myasthenia gravis. Steven-Johnson syndrome, idiopathic sprue, autoimmune inflammatory bowel disease (e.g. ulcerative colitis and Crohn's disease), endocrine opthalmopathy. Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly: atherosclerosis, myocardial infarction, ischemic stroke and congestive heart failure, Alzheimer's disease. Parkinson's disease, amyotrophic lateral sclerosis. Huntington's disease, and cerebral ischemia, and neurodegenerative disease caused by traumatic injury: glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilcad), alpelisib (BYL719, Novartis), tasclisib (GDC-0032. Genentech/Roche); pictilisib (GDC-0941. Genentech/Roche); copanlisib (BAY806946, Bayer); duvelisib (formerly IPI-145. Infinity Pharmaceuticals); PQR309 (Piqur Therapeutics, Switzerland); and TGR1202 (formerly RP5230, TG Therapeutics).

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes: cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies: compounds which target, decrease or inhibit the activity of Flt-3: Hsp90 inhibitors such as 17-AAG (17-allylaminogeldanamycin. NSC330507). 17-DMAG (17-dimethylaminoethylamino-17-demethoxy-geldanamycin. NSC707545), IPI-504. CNF1010. CNF2024. CNF1010 from Conforma Therapeutics: temozolomide (Temodal®); kinesin spindle protein inhibitors, such as SB715992 or SB743921 from GlaxoSmithKline, or pentamidine/chlorpromazine from CombinatoRx: MEK inhibitors such as ARRY142886 from Array BioPharma. AZD6244 from AstraZeneca. PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™, Formestane is marketed under the trade name Lentaron™, Fadrozole is marketed under the trade name Afema™, Anastrozole is marketed under the trade name Arimidex™, Letrozole is marketed under the trade names Femara™ or Femar™, Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues. 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed, under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine. DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199. ABT-731. ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139). HA14-1 (and analogs thereof: see WO2008118802), navitoclax (and analogs thereof, see U.S. Pat. No. 7,390,799). NH-1 (Shenayng Pharmaceutical University), obatoclax (and analogs thereof, see WO2004106328). S-001 (Gloria Pharmaceuticals). TW series compounds (Univ, of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family; or ephrin B4 inhibitors; c) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family; such as compounds which target, decrease or inhibit the activity of the c-Kit receptor tyrosine kinase family; especially compounds which inhibit the c-Kit receptor, such as imatinib; i) compounds targeting, decreasing or inhibiting the activity of members of the c-Abl family; their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporinc derivatives, such as midostaurin; examples of further compounds include UCN-01, safingol, BAY 43-9006, Bryostatin 1, Perifosinc; Ilmofosinc; RO 318220 and RO 320432: GO 6976: Isis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490: Tyrphostin B44; Tyrphostin B44 (+) enantiomer; Tyrphostin AG 555; AG 494; Tyrphostin AG 556. AG957 and adaphostin (4-{[(2,5-dihydroxyphenyl)methyl]amino}-benzoic acid adamantyl ester: NSC 680410, adaphostin); 1) compounds targeting, decreasing or inhibiting the activity of the epidermal growth factor family of receptor tyrosine kinases (EGFR₁ ErbB₂, ErbB₃, ErbB₄ as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB₂, ErbB₃ and ErbB₄ or bind to EGF or EGF related ligands, CP 358774, ZD 1839, ZM 105180; trastuzumab (Herceptin™), cetuximab (Erbitux™), Iressa, Tarceva, OSI-774, C1-1033. EKB-569, GW-2016, E1.1, E2.4, E2.5, E6.2, E6.4, E2.11, E6.3 or E7.6.3, and 7H-pyrrolo-[2,3-d]pyrimidine derivatives: m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAKI/JAK2/JAK3/TYK2 and/or pan-JAK), including but not limited to PRT-062070. SB-1578, baricitinib, pacritinib, momclotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147. XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176. U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity; e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, ctridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™ Alendronic acid is marketed under the trade name Fosamax™. Ibandronic acid is marketed under the trade name Bondranat™. Risedronic acid is marketed under the trade name Actonel™ Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras: for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™); carfilzomib (Kyprolis R, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90: degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative: other geldanamycin related compounds: radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228). Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman. Principles of Radiation Therapy: Cancer, in Principles and Practice of Oncology. Devita et al., Eds., 4th Edition. Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine. 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl) phthalazine or a pharmaceutically acceptable salt thereof. 1-(4-chloroanilino)-4-(4-pyridylmethyl) phthalazine succinate; Angiostatin™; Endostatin™: anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors. FLT-3 inhibitors. VEGFR-2 IgGI antibody; Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone. 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately; before, simultaneously with or after the other drug substance. Accordingly the invention includes a combination of a compound of the invention as hereinbefore described with an anti-inflammatory, bronchodilatory; antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247: LTD4 antagonists such as montelukast and zafirlukast; PDE4 inhibitors such cilomilast (Ariflo®, GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially; formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125. SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy; radiotherapy, immunotherapy, phototherapy; surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy; for example in patients at risk.

Those additional agents may be administered separately from an inventive compound-containing composition, as part of a multiple dosage regimen. Alternatively; those agents may be part of a single dosage form, mixed together with a compound of this invention in a single composition. If administered as part of a multiple dosage regime, the two active agents may be submitted simultaneously, sequentially or within a period of time from one another normally within five hours from one another.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably: compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family: which includes B7-1, B7-2, B7-H1 (PD-L1), B7-DC (PD-L2), B7-H2 (ICOS-L), B7-H3, B7-H4, B7-H5 (VISTA), and B7-H6, Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTBR, LIGHT, DcR3, HVEM, VEGI/TLIA, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-LI, PD-L2, LAG-3, TIM-3, Galcctin 9, CEACAM-1, BTLA, CD69, Galectin-1, TIGIT, CD113, GPR56, VISTA, 2B4, CD48, GARP, PDIH, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-IR antagonists such as CSF-IR antagonist antibodies including RG7155 (WO11/70024, WO11/107553. WO11/131407. WO13/87699. WO13/119716. WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-LI/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514: WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgGI, called AMP-224.

In some embodiments, an immuno-oncology agent is a PD-LI antagonist. In some embodiments, a PD-LI antagonist is an antagonistic PD-LI antibody. In some embodiments, a PD-L1 antibody is MPDL3280A (RG7446: WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS: F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab. In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab. BMS-936559, catumaxomab, durvalumab, epacadostat, epratuzumab, indoximod, inotuzumab ozogamicin, intelumumab, ipilimumab, isatuximab, lambrolizumab. MED14736. MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. Sec, e.g., Okazaki. T. et al. (2013) Nat. Immunol. 14, 1212-1218; Zou et al. (2016) Sci. Transl. Med. 8. The anti-PD-1 antibody nivolumab (OpdivoR. Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygick, Bio Vex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (Pexa Vec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin R, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated; Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8 T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June: hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337. VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15), rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15; sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology.” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood. “Small molecule immuno-oncology therapeutic agents.” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLOS ONE 12 (8); c0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody; a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA. KIR, 2B4 (belongs to the CD2 family of molecules and is expressed on all NK. γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1. B7-H2, B7-H3, B7-H4, B7-H5, B7-H6 and B7-H7, Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1. BTLA. HVEM. TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-LI monoclonal Antibody (Anti-B7-HI; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-LI antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody; Opdivo®, Bristol-Myers Squibb); pembrolizumab (anti-PD-1 antibody; Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011). AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma: avelumab (Bavencio®, Pfizer/Merck KGaA), also known as MSB0010718C), a fully human IgG1 anti-PD-L1 antibody, in clinical trials for non-small cell lung cancer. Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675.206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma, AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3), TIM-3 inhibitors that may be used in the present invention include TSR-022, LY3321367 and MBG453, TSR-022 (Tesaro) is an anti-TIM-3 antibody which is being studied in solid tumors (NCT02817633), LY3321367 (Eli Lilly) is an anti-TIM-3 antibody which is being studied in solid tumors (NCT03099109), MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody; and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566. Pfizer) an agonistic anti-CD137 antibody, in diffuse large B-cell lymphoma (NCT02951156) and in advanced cancers and neoplasms (NCT02554812 and NCT05082566); urelumab (BMS-663513. Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127. Celldex Therapeutics) an agonistic anti-CD27 antibody; in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody; in malignant melanoma and other malignant solid tumors (NCT01239134 and NCT02628574); GWN323 (Novartis), an agonistic anti-GITR antibody; in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015. Innate Pharma/Bristol-Myers Squibb), an anti-KIR antibody, in leukemias (NCT01687387. NCT02399917. NCT02481297. NCT02599649), multiple myeloma (NCT02252263), and lymphoma (NCT01592370); IPH2101 (1-7F9. Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not cat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven. Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815. Aduro Biotech/Novartis), an agonistic synthetic cyclic dinucleotide, in Phase 1 (NCT02675439 and NCT03172936).

In some embodiments, stat3 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-IR antibody, in pancreatic cancer (NCT03153410), melanoma (NCT03101254), and solid tumors (NCT02718911); and BLZ945 (4-[2 ((1R,2R)-2-hydroxycyclohexylamino)-benzothiazol-6-yloxyl]-pyridine-2-carboxylic acid methylamide, Novartis), an orally available inhibitor of CSFIR, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR, Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals: these signals have not been reported in the experimental provided hereinafter.

TABLE 2
Analytical instruments

| | |
|---|---|
| LCMS | Shimadzu UFLC MS: LCMS-2020 |
| | Agilent Technologies |
| | 1200 series MS: |
| | Agilent Technologies 6110 |
| | Agilent Technologies |
| | 1200 series MS: |
| | LC/MSD VL |
| NMR | BRUKER AVANCE |
| | III/400; Frequency (MHz) 400.13; |
| | Nucleus: 1H; |
| | Number of Transients: 8 |
| Prep-HPLC | Gilson GX-281 systems: |
| | instruments GX-A, GX-B, GX-C, GX-D, GX-E, |
| | GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5u. Column flow was 16.0 ml/min. Mobile phase were used (A) 0.1% Formic Acid in Water and (B) Acetonitrile Basic method used (A) 5 mM ammonium bicarbonate and 0.1% NH₃ in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates:

### (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (Intermediate A) (CAS #1948273-03-7)

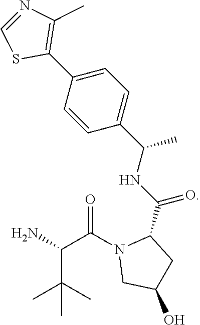 [SMILES: Cc1ncsc1-c1ccc([C@H](C)NC(=O)[C@@H]2C[C@@H](O)CN2C(=O)[C@@H](N)C(C)(C)C)cc1]

### 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (Intermediate B)

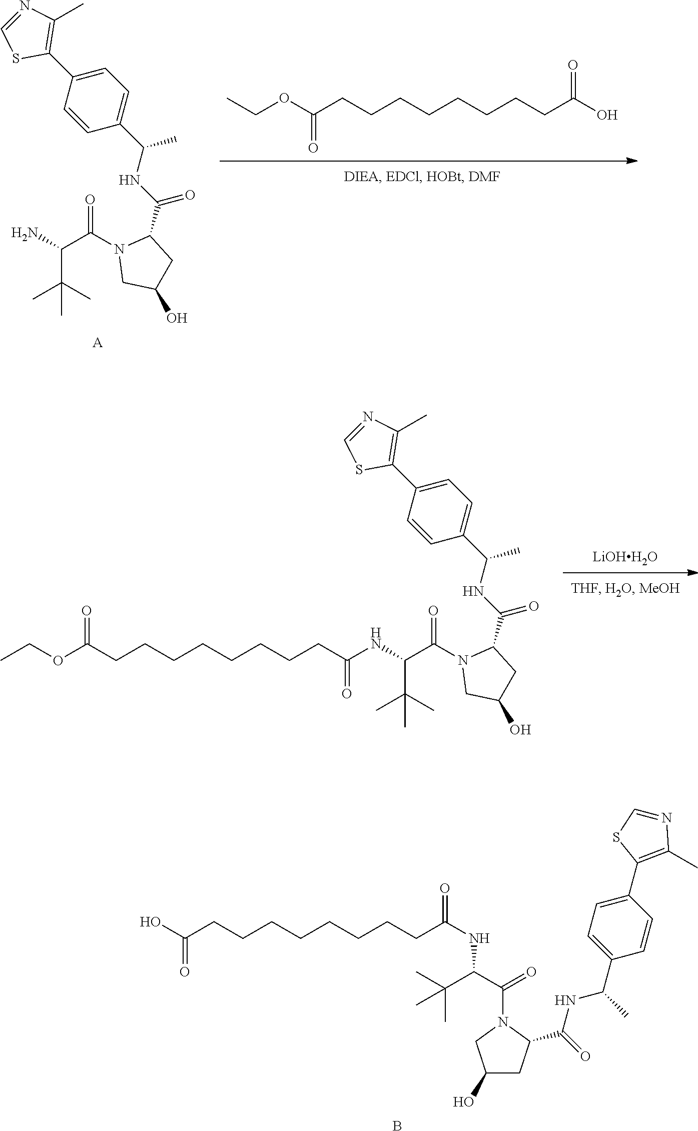

### Step 1—Ethyl 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoate

A mixture of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (2.00 g, 3.86 mmol, 2 HCl salt, Intermediate A), 10-ethoxy-10-oxo-decanoic acid (1.34 g, 5.80 mmol, CAS #693-55-0) and HOBt (783 mg, 5.80 mmol) in DMF (30 mL) was added DIEA (2.50 g, 19.3 mmol, 3.37 mL) and EDCI (1.11 g, 5.80 mmol), then the mixture was stirred at 25° C., for 3 h. On completion, the reaction mixture was quenched by addition H₂O (30 mL), and then extracted with DCM (30 mL×3). The combined organic layers were washed with brine (30 mL×3), dried over Na₂SO₄, filtered and concentrated under reduced pressure to give the title compound (1.90 g) as a white solid. ¹H NMR (400 MHZ, DMSO-d₆) δ=9.00 (s, 1H), 8.38 (d, J=8.0 Hz, 1H), 7.79 (d, J=9.6 Hz, 1H), 7.49-7.34 (m, 5H), 5.79-5.74 (m, 1H), 4.99-4.86 (m, 1H), 4.60-4.47 (m, 1H), 4.42 (t, J=8.0 Hz, 1H), 4.28 (br s, 1H), 2.31-2.22 (m, 4H), 2.07 (br d, J=7.6 Hz, 2H), 1.85-1.75 (m, 1H), 1.57-1.42 (m, 6H), 1.38 (d, J=7.2 Hz, 4H), 1.18 (t, J=7.1 Hz, 12H), 0.94 (s, 11H).

### Step 2—10—(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid

A mixture of ethyl 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoate (1.90 g, 2.89 mmol) in MeOH (6 mL), H₂O (6 mL) and THF (30 mL) was added LiOH·H₂O (486 mg, 11.6 mmol), and then the mixture was stirred at 20° C., for 2 h under N₂ atmosphere. On completion, the reaction mixture was quenched by addition HCl, the pH was adjusted to 3˜4. Then to the mixture was added H₂O (30 mL), and extracted with EtOAc (30 mL×3). The combined organic layers were washed with brine (30 mL×3), dried over Na₂SO₄, filtered and concentrated under reduced pressure to give the title compound (1.60 g) as white solid. ¹H NMR (400 MHZ, DMSO-d₆) δ=12.14-11.69 (m, 1H), 8.98 (s, 1H), 8.37 (d, J=8.0 Hz, 1H), 7.78 (d, J=9.2 Hz, 1H), 7.45-7.42 (m, 2H), 7.40-7.36 (m, 2H), 5.09 (br d, J=3.2 Hz, 1H), 4.92 (br t, J=7.2 Hz, 1H), 4.57-4.48 (m, 1H), 4.42 (t, J=8.0 Hz, 1H), 4.28 (br s, 1H), 3.63-3.58 (m, 2H), 2.46 (s, 4H), 2.18 (t, J=7.4 Hz, 3H), 1.91 (s, 1H), 1.83-1.75 (m, 1H), 1.51-1.45 (m, 4H), 1.38 (d, J=7.2 Hz, 3H), 1.24 (br s, 8H), 0.94 (s, 9H).

### (R)-4-(4-((4′-chloro-4,4-dimethyl-3,4,5,6-tetrahydro-[1, l′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (Intermediate C)

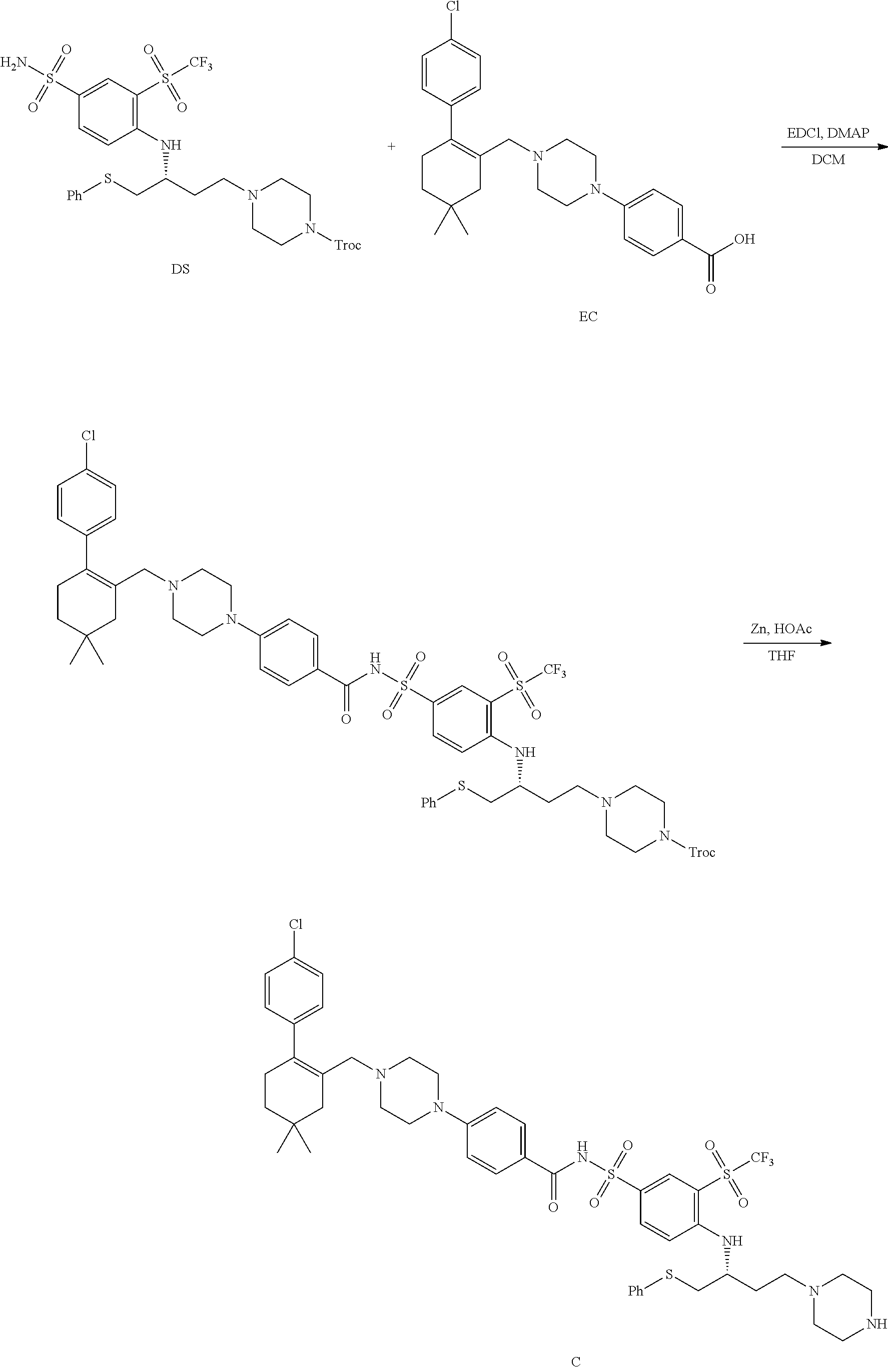

### Step 1—2,2,2-trichloroethyl (R)-4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carboxylate

To a mixture of 2,2,2-trichloroethyl (R)-4-(4-(phenylthio)-3-((4-sulfamoyl-2-((trifluoromethyl)sulfonyl)phenyl)amino)butyl)piperazine-1-carboxylate (17.0 g, 23.4 mmol, Intermediate DS), 4-(4-((4′-chloro-4,4-dimethyl-3,4,5,6-tetrahydro-[1,1′-biphenyl]-2-yl)methyl)piperazin-1-yl)benzoic acid (10.3 g, 23.4 mmol, Intermediate EC), DMAP (5.70 g, 46.6 mmol) and EDCI (8.95 g, 46.7 mmol) in DCM (340 mL) was stirred at 20° C., for 12 h. On completion, the reaction mixture was poured into H₂O (600 mL) at 25° C., and extracted with DCM (300 mL×3). The combined organic layers were washed with brine (600 mL), dried over Na₂SO₄ filtered and concentrated under reduced pressure to give a residue. The residue was purified by prep-HPLC (column: Welch Xtimate C18 250*70 mm #10 um; mobile phase: [water (TFA)-ACN]: B %: 35%-65%, 18 min) to give the title compound (25.5 g, 22.0 mmol, 94% yield) was obtained as an off-white solid. LC-MS (ESI⁺) m/z 1149.5 (M+H)⁺.

### Step 2—(R)-4-(4-((4′-chloro-4,4-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide

To a mixture of 2,2,2-trichloroethyl (R)-4-(3-((4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carboxylate (20.0 g, 17.4 mmol) and CH₃COOH (39.9 g, 665 mmol, 38.0 mL) in THF (400 mL) was added Zn (61.2 g, 936 mmol). Then the reaction mixture was stirred at 20° C., for 12 h. On completion, the solid was removed by filtration, and the filtrate was poured into H₂O (800 mL) at 25° C., then extracted with EtOAc (400 mL×3). The combined organic layers were washed with brine (800 mL), dried over Na₂SO₄ filtered and concentrated under reduced pressure to give a residue. The residue was purified by column chromatography (SiO₂, DCM/MeOH=20/1:5/1, Rf=0.45) and by prep-TLC (SiO₂, DCM:MeOH=5:1) to give the title compound (13.2 g, 76% yield) as a white solid. LC-MS (ESI⁺) m/z 973.2 (M+H)⁺. ¹H NMR (400 MHZ, CDCl₃) δ 8.21 (s, 1H), 7.93 (d, J=9.2 Hz, 1H), 7.85 (d, J=8.4 Hz, 2H), 7.33-7.24 (m, 2H), 7.22-7.15 (m, 6H), 7.15-7.08 (m, 1H), 6.92 (d, J=8.4 Hz, 2H), 6.77 (d, J=8.4 Hz, 1H), 6.66 (d, J=8.8 Hz, 2H), 6.46 (d, J=9.2 Hz, 1H), 3.83-3.67 (m, 1H), 3.17-3.08 (m, 4H), 3.02-2.92 (m, 5H), 2.89-2.78 (m, 1H), 2.72 (s, 2H), 2.64-2.13 (m, 12H), 2.04-1.91 (m, 3H), 1.62-1.49 (m, 1H), 1.39 (t, J=6.4 Hz, 2H), 0.91 (s, 6H).

### (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (Intermediate D)

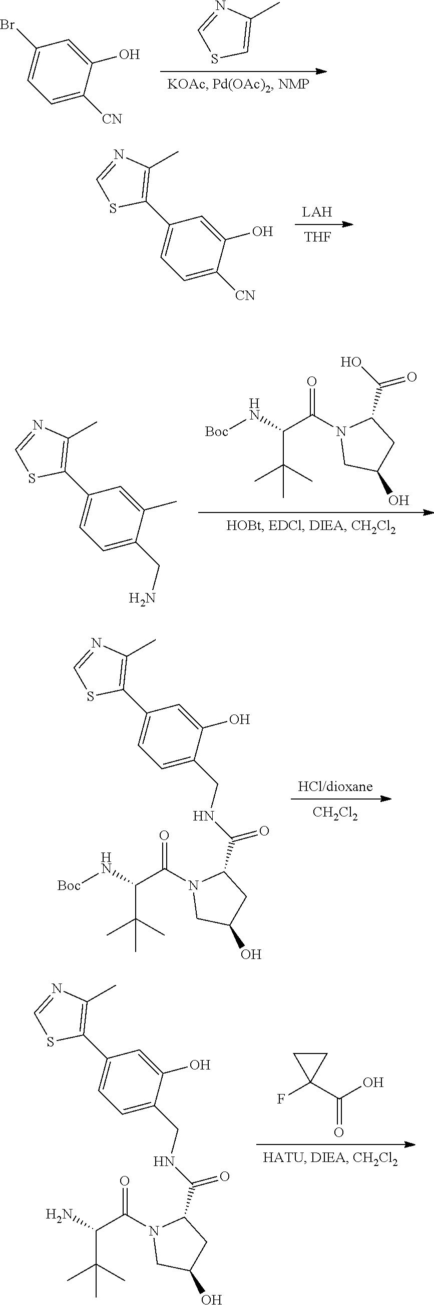

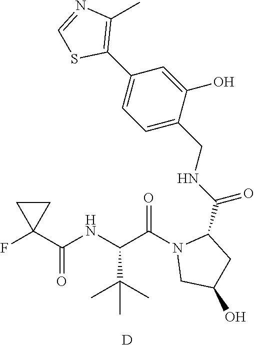

### Step 1—2-hydroxy-4-(4-methylthiazol-5-yl)benzonitrile

To a solution of 4-bromo-2-hydroxybenzonitrile (118 g, 596 mmol) in NMP (1.00 L) was added 4-methylthiazole (177 g, 1.79 mol), KOAc (175 g, 1.79 mol) and Pd (OAc) 2 (2.68 g, 11.9 mmol) and the mixture was stirred at 130° C., for 12 hrs under N₂. On completion, the reaction mixture was diluted with EtOAc (1.50 L) and washed with brine (1.50 L×3), dried over Na₂SO₄, filtered and concentrated under reduced pressure to give the title compound (104 g, 81% yield) as a gray solid. LC-MS (ESI⁺) m/z 217.2 (M+H)⁺.

### Step 2—(2-methyl-4-(4-methylthiazol-5-yl)phenyl)methanamine

A solution of LAH (50.5 g, 1.33 mol) in THF (500 mL) was cooled to 0° C., and then 2-hydroxy-4-(4-methylthiazol-5-yl)benzonitrile (96.0 g, 444 mmol) in THF (1.00 L) was added dropwise at 0° C., under N₂ atmosphere. After the addition, the mixture was stirred at this temperature for 30 min, then warming to 20° C., gradually, and then the mixture was stirred at 50° C., for 2 hrs under N₂. On completion, the reaction mixture was quenched with H₂O (50.0 mL), NaOH (aq, 15%) (50.0 mL) and H₂O (150 mL) at 0° C. The mixture was then filtered and the filter cake was washed with THF (300 mL×3). The filtrated was then concentrated under reduced pressure to give the title compound (70.0 g, 72% yield) as a yellow solid. LC-MS (ESI) m/z 221.2 (M+H).

### Step 3—Tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl) carbamate

To a solution of (2-methyl-4-(4-methylthiazol-5-yl)phenyl)methanamine (28.0 g, 108 mmol) in CH₂Cl₂ (400 mL) was added HOBt (16.1 g, 119 mmol), EDCI (22.8 g, 119 mmol), DIEA (41.9 g, 324 mmol) and (2S,4R)-1-((S)-2-((tert-butoxycarbonyl)amino)-3,3-dimethylbutanoyl)-4-hydroxypyrrolidine-2-carboxylic acid (37.2 g, 108 mmol. CAS #630421-46-4). The mixture was the stirred at 25° C., for 18 hrs. On completion, the reaction mixture was diluted with CH₂Cl₂ (300 mL) and the combined organic layers were washed with citric acid (200 mL×2). NaHCO; (aq. 100%) (200 mL×2) and brine (200 mL×2). The organic layer was then ried over Na₂SO₄, filtered and concentrated under reduced pressure to give a residue. The residue was purified by column chromatography (SiO₂, CH₂Cl₂/EtOAC=1/0 to 1/1) to give the title compound (28.0 g, 47% yield) as a yellow solid. ¹H NMR (400 MHZ. MeOD) δ 8.85 (s, 1H), 7.36 (d, J=8.4 Hz, 1H), 6.84-6.95 (m, 2H), 4.60 (br t, J=8.0 Hz, 1H), 4.50 (br s, 1H), 4.35 (br d, J=4.8 Hz, 2H). 4.25-4.32 (m, 1H), 3.76-3.91 (m, 2H), 2.45-2.50 (m. 3H), 2.07-2.24 (m, 2H), 1.43 (s, 9H), 1.00 (s, 9H).

### Step 4—(2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide

To a solution of Tert-butyl ((S)-1-((2S,4R)-4-hydroxy-2-((2-hydroxy-4-(4-methylthiazol-5-yl)benzyl)carbamoyl)pyrrolidin-1-yl)-3,3-dimethyl-1-oxobutan-2-yl) carbamate (50.0 g, 91.5 mmol) in CH₂Cl₂ (500 mL) was added HCl/dioxane (4 M. 165 mL) dropwise to the reaction and the mixture was stirred at 25° C., for 0.5 hrs. On completion, the reaction mixture was concentrated under reduced pressure to give the title compound (50 g. HCl) as a yellow solid. LC-MS (ESI) m/z 447.2 (M+H).

### Step 5—(2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide

To a solution of (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (44.0 g, 82.0 mmol. HCl) in CH₂Cl₂ (500 mL) was added HATU (37.4 g, 98.4 mmol). DIEA (31.8 g, 246 mmol) and 1-fluorocyclopropane-1-carboxylic acid (8.53 g, 82.0 mmol). The mixture was stirred at 30° C., for 2.5 hrs. On completion, the reaction mixture was diluted with CH₂Cl₂ (200 mL) and the combined organic layers were washed with citric acid (400 mL×2). NaHCO; (aq. 100%) (400 mL×2) and brine (400 mL×2), dried over Na₂SO₄, filtered and concentrated under reduced pressure to give a residue. The residue was purified by column chromatography (SiO₂, CH₂Cl₂/EtOAc=I/O to 1/3) to give the title compound. ¹H NMR: (400 MHZ. CDCl₃) δ 9.31 (br s, 1H), 8.67 (s, 1H), 8.19 (br t, J=6.0 Hz, 1H), 7.09 (d, J=7.6 Hz, 1H), 6.98-7.02 (m, 1H), 6.95 (s, 1H), 6.84 (br d, J=7.6 Hz, 1H), 4.67 (br t, J=8.0 Hz, 1H), 4.44-4.51 (m, 2H), 4.35-4.41 (m, 1H), 4.12-4.17 (m, 1H), 3.96 (br d, J=11.2 Hz, 1H), 3.59-3.63 (m, 1H), 2.49 (s, 3H), 2.33-2.40 (m, 1H), 2.00-2.08 (m, 1H), 1.19-1.35 (m, 4H), 0.92 (s, 9H). LC-MS (ESI⁺) m/z 533.2 (M+H)⁺.

### Methyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)butanoate (Intermediate E)

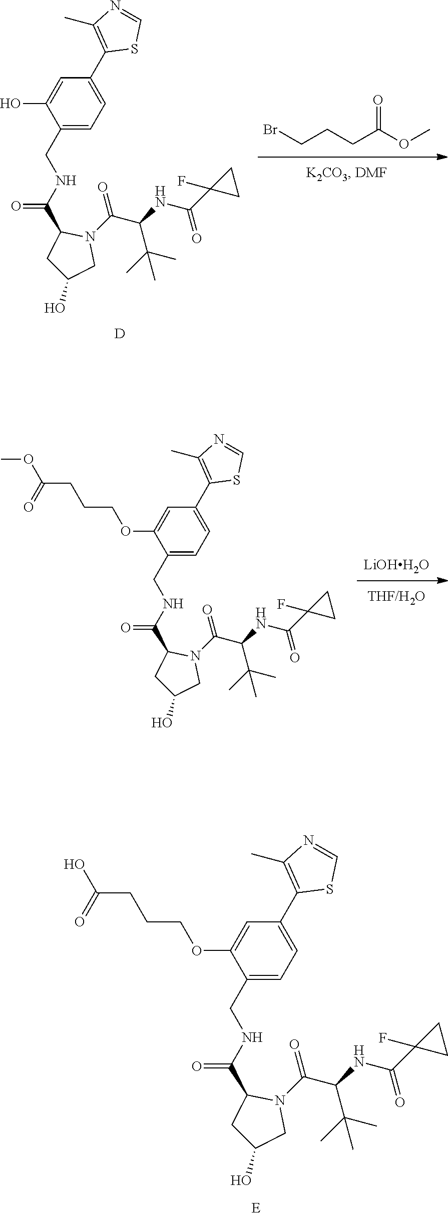

### Step 1—Methyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)butanoate

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (1.5 g, 2.82 mmol, Intermediate D) and methyl 4-bromobutanoate (764 mg, 4.22 mmol) in DMF (30 mL) was added K₂CO₃ (1.17 g, 8.45 mmol) at 20° C., under nitrogen flow. Then the reaction was stirred at 70° C., for 10 h under nitrogen atmosphere. On completion, the reaction was poured into water (50 mL) and extracted with EtOAc (40 mL×2). The combined organic phase is washed with brine (30 mL×2), and dried over Na₂SO₄. Then the mixture was filtered to get the filtrate and concentrated to give a residue. The residue was purified by column chromatography on silica gel (eluted with petroleum ether:ethyl acetate=100:1 to 100:60) to give the title compound (950 mg, 53% yield) as a yellow oil. ¹H NMR (400 MHZ, DMSO-d₆) δ=8.99 (s, 1H), 8.49 (t, J=5.9 Hz, 1H), 7.41 (d, J=7.6 Hz, 1H), 7.29 (dd, J=2.4, 9.2 Hz, 1H), 7.00 (d, J=1.2 Hz, 1H), 6.96 (dd, J=1.2, 7.6 Hz, 1H), 5.17 (d, J=3.6 Hz, 1H), 4.60 (d, J=9.2 Hz, 1H), 4.52 (t, J=8.4 Hz, 1H), 4.36 (br s, 1H), 4.32-4.15 (m, 2H), 4.08 (t, J=6.0 Hz, 2H), 3.70-3.63 (m, 1H), 3.61 (s, 4H), 2.56-2.52 (m, 3H), 2.46 (s, 3H), 2.03 (br t, J=6.4 Hz, 2H), 1.97-1.87 (m, 1H), 1.42-1.32 (m, 2H), 1.22 (br dd, J=3.2, 8.4 Hz, 2H), 0.96 (s, 9H).

### Step 2—Methyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)butanoate

To a solution of methyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)butanoate (400 mg, 632 umol) in THF (8 mL) and H₂O (2 mL) was added LiOH·H₂O (53.0 mg, 1.26 mmol) at 20° C., under nitrogen flow. Then the reaction was stirred at 20° C., for 2 h under nitrogen atmosphere. On completion, the reaction was poured into water (10 mL) and acidified with 1N HCl to pH=4 then extracted with EtOAc (10 mL×2). The combined organic phase is washed with brine (3 mL×2), and dried over Na₂SO₄. Then the mixture was filtered to get the filtrate and concentrated to give the title compound (0.35 g, 89.4% yield) as a yellow solid. ¹H NMR (400 MHZ, DMSO-d₆) δ=12.12 (br s, 1H), 8.98 (s, 1H), 8.50 (br t, J=6.0 Hz, 1H), 7.42 (d, J=7.6 Hz, 1H), 7.30 (dd, J=2.4, 9.2 Hz, 1H), 7.05-6.99 (m, 1H), 6.99-6.92 (m, 1H), 5.17 (d, J=3.6 Hz, 1H), 4.60 (d, J=9.2 Hz, 1H), 4.52 (t, J=8.0 Hz, 1H), 4.40-4.27 (m, 2H), 4.25-4.16 (m, 1H), 4.11-4.07 (m, 2H), 3.72-3.56 (m, 2H), 2.46 (s, 3H), 2.44-2.40 (m, 2H), 2.09 (br dd, J=7.6, 12.4 Hz, 1H), 2.04-2.00 (m, 2H), 1.98-1.88 (m, 3H), 1.43-1.38 (m, 1H), 1.37-1.32 (m, 1H), 1.26-1.20 (m, 2H), 0.97 (s, 9H).

### 7-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)heptanoic acid (Intermediate F)

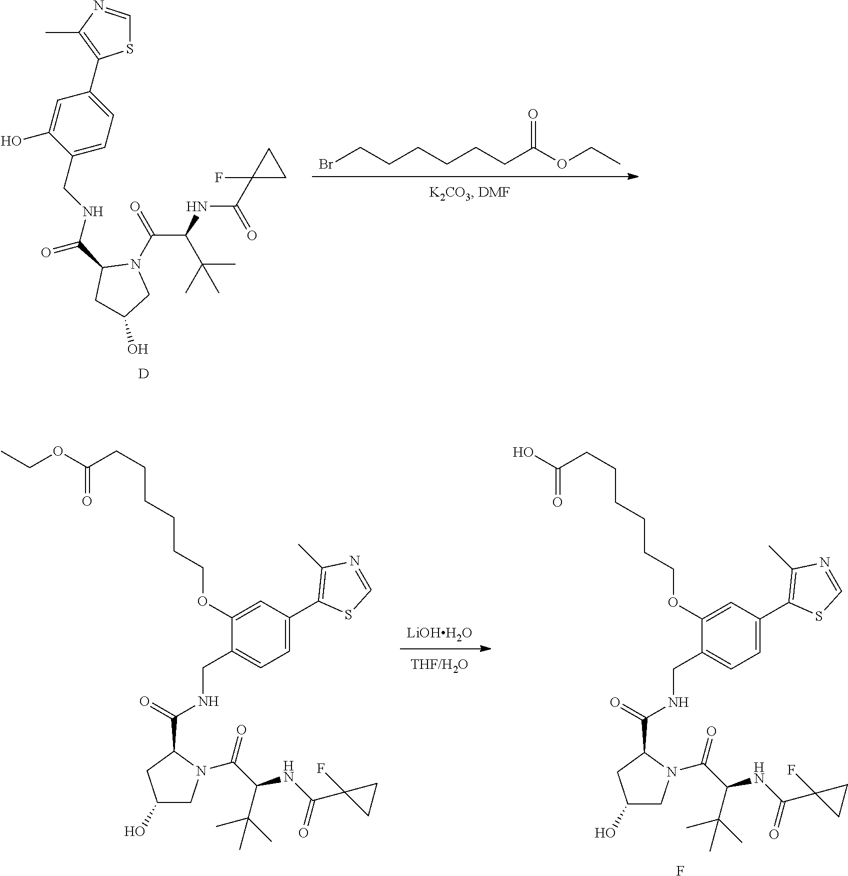

### Step 1 Ethyl 7-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)heptanoate

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (1.5 g, 2.82 mmol, Intermediate D) and ethyl 7-bromoheptanoate (1.00 g, 4.22 mmol, CAS #29823-18-5) in DMF (30 mL) was added K₂CO; (1.17 g, 8.45 mmol) at 20° C., under nitrogen flow. Then the reaction was stirred at 70° C. for 10 h under nitrogen atmosphere. On completion, the reaction was poured into water (40 mL) and extracted with EtOAc (30 mL×2). The combined organic phase is washed with brine (30 mL×2), and dried over Na₂SO₄. Then the mixture was filtered to get the filtrate and concentrated to give a residue. The residue was purified by column chromatography on silica gel (eluted with petroleum ether:ethyl acetate=100:1 to 100:70) to give the title compound (1 g, 52% yield) as a white solid. ¹H NMR (400 MHZ, DMSO-d₆) δ=8.98 (s, 1H), 8.48 (t, J=6.0 Hz, 1H), 7.40 (d, J=7.6 Hz, 1H), 7.28 (dd, J=2.4, 9.2 Hz, 1H), 7.02-6.98 (m, 1H), 6.94 (dd, J=1.6, 7.6 Hz, 1H), 5.16 (d, J=3.6 Hz, 1H), 4.59 (d, J=9.2 Hz, 1H), 4.51 (t, J=8.4 Hz, 1H), 4.35 (br s, 1H), 4.32-4.24 (m, 1H), 4.24-4.15 (m, 1H), 4.05-4.01 (m, 4H), 3.70-3.57 (m, 2H), 2.45 (s, 3H), 2.29 (t, J=7.3 Hz, 2H), 2.08 (br dd, J=7.6, 12.6 Hz, 1H), 1.97-1.88 (m, 1H), 1.79-1.70 (m, 2H), 1.56 (quin, J=7.2 Hz, 2H), 1.50-1.42 (m, 2H), 1.41-1.30 (m, 4H), 1.25-1.20 (m, 2H), 1.17 (d, J=4.4 Hz, 3H), 0.96 (s, 9H).

### Step 2—7-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)heptanoic acid

To a solution of ethyl 7-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)heptanoate (500 mg, 725 umol) in THF (8 mL) and H₂O (2 mL) was added LiOH·H₂O (60.9 mg, 1.45 mmol) at 20° C., under nitrogen flow. Then the reaction was stirred at 20° C., for 2 h under nitrogen atmosphere. On completion, the reaction was poured into water (10 mL) and extracted with EtOAc (10 mL×2). The aqueous phase was acidified with 1N HCl to pH=4, then extracted with EtOAc (10 mL×2). The combined organic phase is washed with brine (3 mL×2), and dried over Na₂SO₄. Then the mixture was filtered to get the filtrate and concentrated to give the title compound (450 mg, 89% yield) as a yellow solid. LC-MS (ESI⁺) m/z 661.3 (M+H)⁺.

### (2S,4R)-1-((S)-3,3-dimethyl-2-(4-(methylamino) butanamido)butanoyl)-4-hydroxy-N-((S)-1-(4-(4-methylthiazol-5-yl)phenyl)ethyl)pyrrolidine-2-carboxamide (Intermediate G)

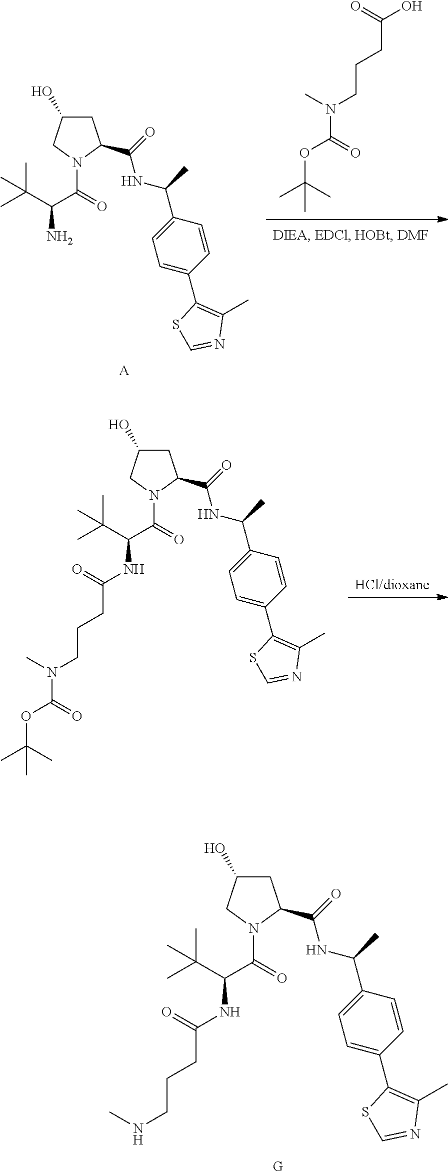

### Step 1—Tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamate

A mixture of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (2.00 g, 3.86 mmol, 2HCI salt, Intermediate A), 4-((tert-butoxycarbonyl)(methyl)amino) butanoic acid (1.26 g, 5.80 mmol, CAS #94994-39-5) and DIEA (2.50 g, 19.3 mmol) in DMF (20 mL) was added HOBt (783 mg, 5.80 mmol), and EDCI (1.11 g, 5.80 mmol). The mixture was stirred at 25° C., for 12 h. On completion, the reaction mixture was diluted with water (50 mL), and extracted with DCM (50 mL×3). The combined organic layers were washed with brine (30 mL×4), dried over Na₂SO₄ and evaporated. The residue was purified by column chromatography (SiO₂, Petroleum ether/Ethyl acetate=0/1 to DCM:MeOH=10:1) to give the title compound (2.30 g, 92.4% yield) as a white solid. ¹H NMR (400 MHZ, DMSO-d₆) δ=8.98 (s, 1H), 8.36 (d, J=8.0 Hz, 1H), 7.88 (br d, J=9.2 Hz, 1H), 7.45-7.41 (m, 2H), 7.40-7.36 (m, 2H), 5.08 (d, J=3.6 Hz, 1H), 4.95-4.88 (m, 1H), 4.51 (br d, J=9.2 Hz, 1H), 4.42 (t, J=8.0 Hz, 1H), 4.28 (br d, J=3.2 Hz, 1H), 3.61 (br d, J=4.0 Hz, 2H), 3.12 (br s, 2H), 2.75 (br s, 3H), 2.45 (s, 3H), 2.25-2.17 (m, 1H), 2.13-2.07 (m, 1H), 2.04-1.99 (m, 1H), 1.79 (ddd, J=4.4, 8.4, 12.8 Hz, 1H), 1.70-1.62 (m, 2H), 1.39-1.36 (m, 12H), 0.95-0.92 (m, 9H)

### Step 2—(2S,4R)-1-((S)-3,3-dimethyl-2-(4-(methylamino) butanamido)butanoyl)-4-hydroxy-N-((S)-1-(4-(4-methylthiazol-5-yl)phenyl)ethyl)pyrrolidine-2-carboxamide

A mixture of tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)carbamate (2.10 g, 3.26 mmol) in HCl/dioxane (25 mL) was stirred at 20° C., for 0.5 h. On completion, the reaction mixture was filtered and concentrated under reduced pressure to give the title compound (3.10 g, HCl, salt) as a yellow solid. LC-MS (ESI+) m/z 544.3 (M+H)⁺.

### (2S,4R)-1-((S)-3,3-dimethyl-2-(4-(N-methylhex-5-ynamido)butanamido)butanoyl)-4-hydroxy-N-((S)-1-(4-(4-methylthiazol-5-yl)phenyl)ethyl)pyrrolidine-2-carboxamide (Intermediate H)

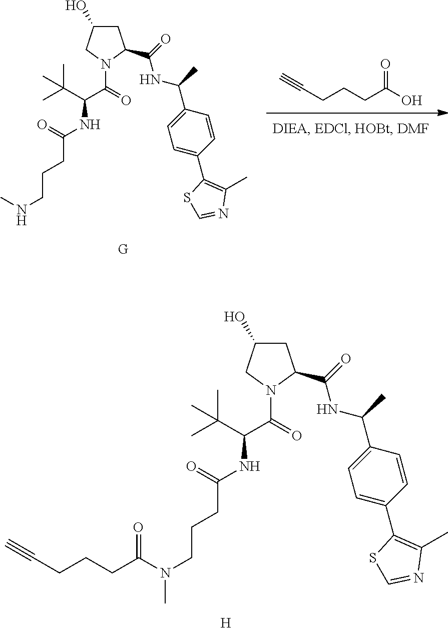

### Step 1—(2S,4R)-1-((S)-3,3-dimethyl-2-(4-(N-methylhex-5-ynamido)butanamido)butanoyl)-4-hydroxy-N-((S)-1-(4-(4-methylthiazol-5-yl)phenyl)ethyl)pyrrolidine-2-carboxamide

A mixture of (2S,4R)-1-((S)-3,3-dimethyl-2-(4-(methylamino) butanamido)butanoyl)-4-hydroxy-N-((S)-1-(4-(4-methylthiazol-5-yl)phenyl)ethyl)pyrrolidine-2-carboxamide (500 mg, 0.862 mmol, HCl, Intermediate G), hex-5-ynoic acid (145 mg, 1.29 mmol) and HOBt (175 mg, 1.29 mmol) in DMF (7.5 mL) was added DIEA (557 mg, 4.31 mmol) and EDCI (248 mg, 1.29 mmol), then the mixture was stirred at 25° C., for 3 h. On completion, the reaction mixture was quenched with water (10 mL), and extracted with DCM (10 mL×3). The combined organic layers were washed with brine (10 mL×2), dried over Na₂SO₄ and evaporated. The residue was purified by flash silica gel chromatography (ISCOR: 12 g SepaFlash® Silica Flash Column, Eluent of 0˜9% Methanol/Dichloromethane ethergradient (40 mL/min) to give the title compound (400 mg, 66% yield) as a white solid. ¹H NMR (400 MHZ, DMSO-d₆) δ=8.98 (s, 1H), 8.37 (br d, J=8.0 Hz, 1H), 7.89 (br dd, J=9.2, 16.8 Hz, 1H), 7.45-7.42 (m, 2H), 7.39-7.36 (m, 2H), 5.09 (t, J=2.8 Hz, 1H), 4.91 (br t, J=7.2 Hz, 1H), 4.52 (br t, J=8.9 Hz, 1H), 4.42 (t, J=8.0 Hz, 1H), 4.28 (br s, 1H), 3.60 (br s, 2H), 3.26-3.22 (m, 2H), 2.95-2.90 (m, 2H), 2.79-2.75 (m, 3H), 2.45 (s, 4H), 2.39-2.32 (m, 3H), 2.18 (td, J=3.2, 6.8 Hz, 4H), 1.68-1.64 (m, 3H), 1.37 (d, J=7.2 Hz, 3H), 0.94 (s, 9H).

### Tert-butyl 6-(8-(benzo[d]thiazol-2-ylcarbamoyl)-3,4-dihydroisoquinolin-2 (1H)-yl)-3-bromopicolinate (Intermediate I)

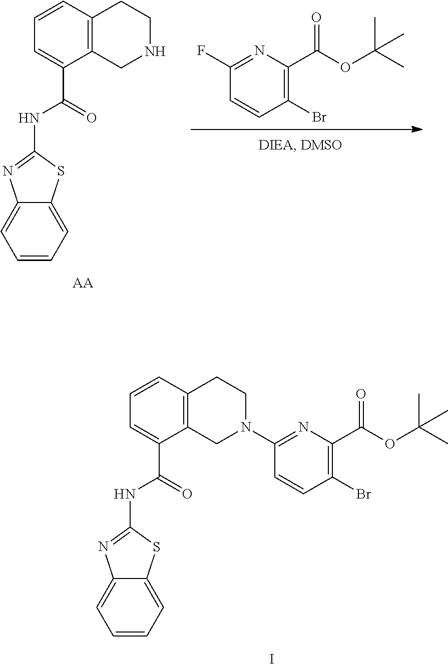

To a solution of N-(benzo[d]thiazol-2-yl)-1,2,3,4-tetrahydroisoquinoline-8-carboxamide (30.0 g, 86.7 mmol, 2HCI, Intermediate AA) and tert-butyl 3-bromo-6-fluoropicolinate (21.7 g, 78.8 mmol, CAS #1430753-76-6) in DMSO (140 mL) was added DIEA (16.3 g, 126 mmol, 21.9 mL). The mixture was stirred at 95° C., for 12 hrs. On completion, the reaction mixture was diluted with water (420 mL) and extracted with ethyl acetate (150 mL×3). The combined organic layers were washed with water (50.0 mL×3), dried over Na₂SO₄, filtered and concentrated under reduced pressure to give a residue. The crude product was triturated with sodium hydroxide solution at rt for 3 hrs. The mixture was filtered to remove the filter liquor. The solide was concentrated in vacuo to give the title compound (25.0 g, 49% yield) as a brown solid. LC-MS (ESI+) m/z 567.0 (M+H)⁺. ¹H NMR (400 MHZ, DMSO) δ 12.8 (s, 1H), 8.02 (d, J=7.6 Hz, 1H), 7.77 (t, J=6.0 Hz, 2H), 7.58 (d, J=7.6 Hz, 1H), 7.42-7.35 (m, 4H), 6.86 (d, J=9.2 Hz, 1H), 4.92 (s, 1H), 3.77 (t, J=6.0 Hz, 2H), 3.00 (t, J=6.0 Hz, 2H), 1.34 (s, 9H).

### (2S,4R)-1-((S)-3,3-dimethyl-2-(4-(N-methyldec-9-ynamido)butanamido)butanoyl)-4-hydroxy-N-((S)-1-(4-(4-methylthiazol-5-yl)phenyl)ethyl)pyrrolidine-2-carboxamide (Intermediate J)

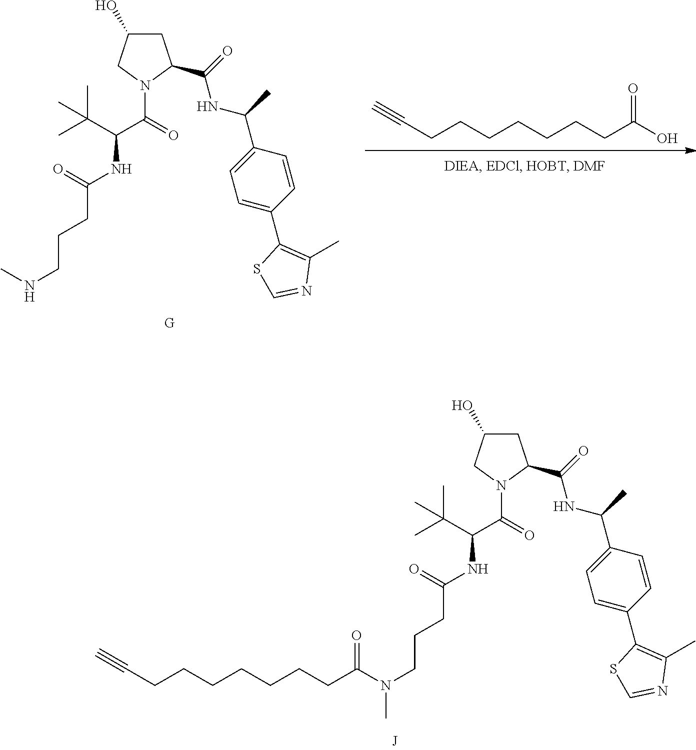

To a solution of (2S,4R)-1-((S)-3,3-dimethyl-2-(4-(methylamino) butanamido)butanoyl)-4-hydroxy-N-((S)-1-(4-(4-methylthiazol-5-yl)phenyl)ethyl)pyrrolidine-2-carboxamide (1.00 g, 1.72 mmol, HCl salt, Intermediate G), dec-9-ynoic acid (435 mg, 2.59 mmol, CAS #1642-49-5) and HOBt (349 mg, 2.59 mmol) in DMF (15 mL) was added DIEA (1.11 g, 8.62 mmol, 1.50 mL) and EDCI (495.6 mg, 2.59 mmol), then the mixture was stirred at 25° C., for 17 h. On completion, the reaction mixture was quenched by addition NH₄Cl (10 mL) and extracted with EtOAC (15 mL×2). The combined organic layers were washed with brine (10 mL×3), dried over Na₂SO₄ and evaporated. The residue was prep-TLC (Petroleum ether/Ethyl acetate=0:1 to DCM:MeOH=10:1) to give the title compound (720 mg, 60.2% yield) as a white solid. ¹H NMR (400 MHZ, DMSO-d₆) δ 8.98 (s, 1H), 8.36 (d, J=7.6 Hz, 1H), 7.89 (dd, J=9.2, 18.8 Hz, 1H), 7.45-7.42 (m, 2H), 7.39-7.36 (m, 2H), 5.75 (s, 1H), 5.09 (dd, J=2.0, 3.2 Hz, 1H), 4.91 (br t, J=7.2 Hz, 1H), 4.52 (dd, J=9.6, 11.6 Hz, 1H), 4.42 (t, J=8.0 Hz, 1H), 4.28 (br d, J=2.4 Hz, 1H), 3.60 (br s, 2H), 3.23 (br t, J=7.2 Hz, 3H), 2.93-2.90 (m, 2H), 2.80-2.75 (m, 2H), 2.45 (s, 4H), 2.25 (br t, J=7.2 Hz, 3H), 2.14 (dt, J=2.8, 6.8 Hz, 4H), 1.79 (ddd, J=4.4, 8.4, 12.8 Hz, 1H), 1.74-1.68 (m, 1H), 1.65-1.60 (m, 1H), 1.46 (br dd, J=7.2, 15.6 Hz, 4H), 1.26 (br d, J=4.0 Hz, 6H), 0.94 (s, 9H).

### 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic (Intermediate K) acid

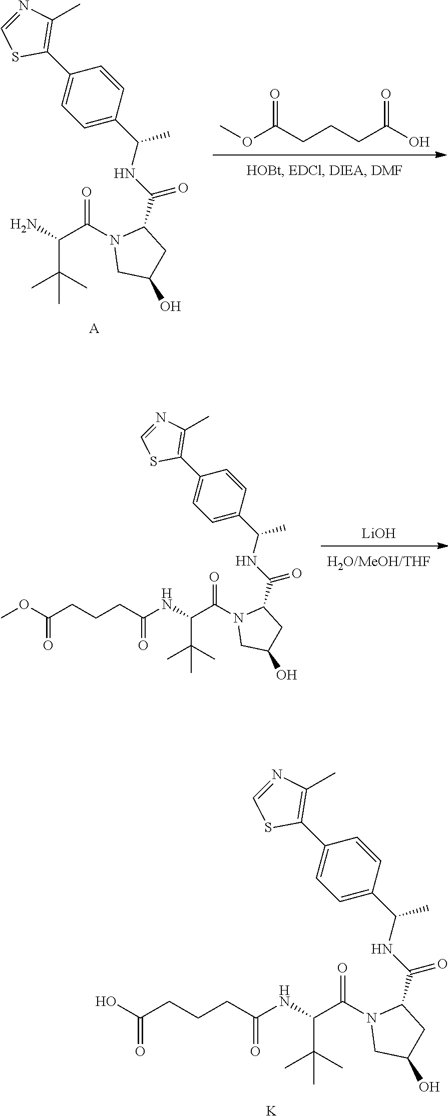

### Step 1—Methyl 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoate

To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (2 g, 3.86 mmol, 2 HCl, Intermediate A), 5-methoxy-5-oxo-pentanoic acid (847 mg, 5.80 mmol, CAS #1501-27-5) and DIEA (2.50 g, 19.3 mmol) in DMF (30 mL) was added HOBt (783 mg, 5.80 mmol) and EDCI (1.11 g, 5.80 mmol), then the mixture was stirred at 25° C., for 12 h. On completion, the reaction mixture was quenched with H₂O (20 mL) at 0° C., and extracted with DCM (10 mL×3). The combined organic layers were washed with salt solution (10 mL×3), dried over sodium sulfate, filtered and concentrated. The residue was purified by column chromatography (SiO₂, Petroleum ether/Ethyl acetate=1/1-0/1) to give the title compound (1.9 g, 86% yield) as a white solid. LC-MS (ESI⁺) m/z 573.5 (M+H)⁺.

### Step 2 5—(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid

To a solution of methyl 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoate (1.9 g, 3.3 mmol) in THF (30 mL), MeOH (6 mL) and H₂O (6 mL) was added LiOH (318 mg, 13.3 mmol), then the mixture was stirred at 20° C., at 2 h. On completion, the reaction mixture was quenched with H₂O (20 mL) and extracted with EtOAC (10 mL×3). The aqueous phase by addition HCl the pH was adjusted to 3˜4, then the mixture was extracted with EtOAC (10×3). The combined organic layers were washed with brine (10 mL×3), dried over Na₂SO₄ and evaporated to give the title compound (1.3 g) as a white solid. LC-MS (ESI+) m/z 559.4 (M+H)⁺.

### 2-(3-hydroxyisoxazol-5-yl)-3-methylbutanoic acid (Intermediate L)

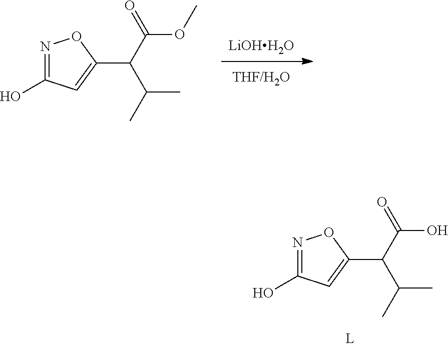

To a solution of methyl 2-(3-hydroxyisoxazol-5-yl)-3-methylbutanoate (9.00 g, 45.2 mmol, CAS #2104986-10-7) in H₂O (18 mL) and THF (90 mL) was added LiOH·H₂O (3.79 g, 90.4 mmol). The mixture was stirred at 25° C., for 2 h. On completion, the reaction mixture was diluted with water (5 mL) and the pH was adjusted to 4˜3 by addition of 2 M HCl, and extracted with EtOAc (20 mL×3). The combined organic layers were washed with brine (10 mL×3), dried over Na₂SO₄ and evaporated to afford title compound (7.70 g) as a white solid. ¹H NMR (400 MHZ, DMSO-d₆) δ=12.33-11.58 (m, 1H), 5.90 (s, 1H), 3.43 (d, J=8.8 Hz, 1H), 2.24 (td, J=6.8, 8.8 Hz, 1H), 0.94 (d, J=6.8 Hz, 3H), 0.83 (d, J=6.8 Hz, 3H).

### (2S,4R)-4-hydroxy-N-(4-(4-methylthiazol-5-yl)benzyl)pyrrolidine-2-carboxamide (Intermediate M)

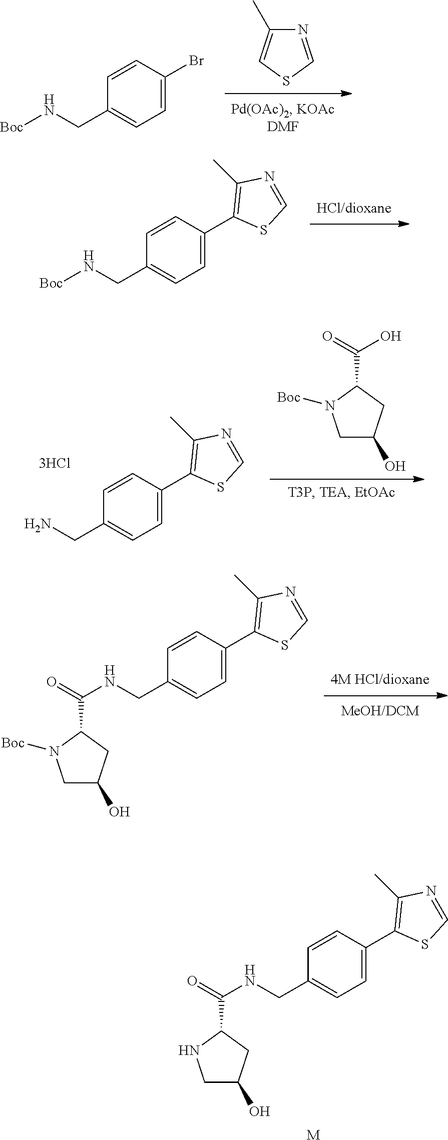

### Step 1—Tert-butyl 4-(4-methylthiazol-5-yl)benzylcarbamate

To a solution of tert-butyl 4-bromobenzylcarbamate (120 g, 416 mmol, CAS #68819-84-1) and 4-methylthiazole (83.0 g, 837 mmol, CAS #693-95-8), and KOAc (81.7 g, 832 mmol) in DMF (1.20 L) under N₂ was added Pd (OAc) 2 (1.87 g, 8.33 mmol) at 20° C., then the mixture was stirred at 90° C., for 16 h. On completion, the mixture was diluted with H₂O (2.00 L) and extracted with EtOAc (600 mL×3). The combined organic layers were washed with brine (500 mL), dried over Na₂SO₄, filtered and concentrated under reduced pressure to give a residue. The crude product was triturated with PE:DCM=3:1 (480 mL) at 25° C., to give the title compound (83.0 g) as a gray solid. LC-MS (ESI+) m/z 305.1 (M+H)⁺.

### Step 2—(4-(4-Methylthiazol-5-yl)phenyl)methanamine

A solution of tert-butyl 4-(4-methylthiazol-5-yl)benzylcarbamate (83.0 g, 257 mmol) in HCl/dioxane (4 M, 250 mL) was stirred at 0° C., for 0.5 h. On completion, the mixture was concentrated under reduced pressure to give the title compound (83 g, 3HCI) as a yellow solid, LC-MS (ESI+) m/z 205.0 (M+H)⁺.

### Step 3 (2S,4R)-tert-butyl 4-hydroxy-2-((4-(4-methylthiazol-5-yl)benzyl)carbamoyl)pyrrolidine-1-carboxylate

To a solution of (4-(4-methylthiazol-5-yl)phenyl)methanamine (80.6 g, 257 mmol, 3 HCl) and (2S,4R)-1-(tert-butoxycarbonyl)-4-hydroxypyrrolidine-2-carboxylic acid (96.0 g, 415 mmol, CAS #13726-69-7) in EtOAc (400 mL) was added T3P (327 g, 513 mmol, 50% solution) and TEA (232 g, 2.29 mol) at 0° C., then the mixture was stirred at 25° C., for 12 h. On completion, the mixture was diluted with H₂O (1.00 L) and extracted with EtOAc (300 mL×3). The combined organic layers were washed with H₂O (500 mL×2), brine (500 mL), dried over Na₂SO₄, filtered and concentrated under reduced pressure to give the title compound (60 g, 53% yield) as an off-white solid. LC-MS (ESI+) m/z 418.2 (M+H)⁺.

### Step 4—(2S,4R)-4-hydroxy-N-(4-(4-methylthiazol-5-yl)benzyl)pyrrolidine-2-carboxamide

To a solution of (2S,4R)-tert-butyl 4-hydroxy-2-((4-(4-methylthiazol-5-yl)benzyl)carbamoyl)pyrrolidine-1-carboxylate (60.0 g, 137 mmol) in DCM (300 mL) and MeOH (120 mL) was added HCl/dioxane (4 M, 70.0 mL) at 0° C., then the mixture was stirred at 25° C., for 4 h. On completion, the mixture was concentrated under reduced pressure to give a residue. The crude product was diluted with MeOH (1.50 L) and alkaline resin (250 g) was added, and the mixture was stirred at 25° C., for 4 h. The mixture was then filtered and concentrated under reduced pressure to give the title compound (50.0 g) as an off-white solid. LC-MS (ESI+) m/z 318.4 (M+H)⁺.

### (2S,4R)-4-hydroxy-1-(2-(3-hydroxyisoxazol-5-yl)-3-methylbutanoyl)-N-(4-(4-methylthiazol-5-yl)benzyl)pyrrolidine-2-carboxamide (Intermediate N)

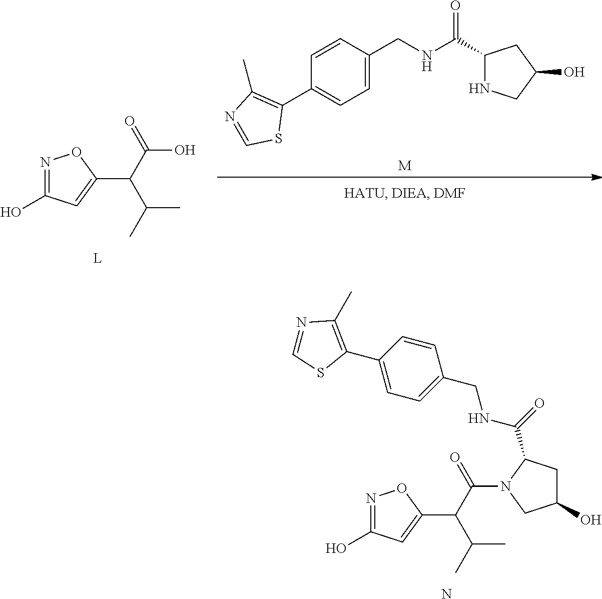

A solution of 2-(3-hydroxyisoxazol-5-yl)-3-methyl-butanoic acid (7.50 g, 40.5 mmol, Intermediate L) and HATU (14.1 g, 37.1 mmol) in DMF (75 mL) was stirred at 25° C., for 0.5 h. Then a solution of (2S,4R)-4-hydroxy-N-(4-(4-methylthiazol-5-yl)benzyl)pyrrolidine-2-carboxamide (10.7 g, 33.75 mmol, Intermediate M) and DIEA (13.1 g, 101 mmol) in DMF (35 mL) was added to the mixture and the mixture was stirred at 25° C., for 2 h. On completion, the reaction mixture was purified by reversed-phase HPLC [I.D. 100 mm*H₃₅₀ mm Welch Ultimate XB_C18 20-40 μm: 120 A: H₂O (NH₃, H₂O)⁺ ACN, 5-30% 20 min: 30% 10 min] to afford title compound (15.7 g, 96% yield) as a yellow solid. ¹H NMR (400 MHZ, DMSO-d₆) δ=8.98 (s, 1H), 8.54-8.41 (m, 1H), 7.46-7.32 (m, 5H), 5.85 (d, J=18.8 Hz, 1H), 5.18-4.98 (m, 1H), 4.42 (br dd, J=7.6, 14.4 Hz, 1H), 3.76 (dd, J=4.4, 10.4 Hz, 1H), 3.69 (d, J=8.4 Hz, 1H), 3.56 (d, J=9.6 Hz, 2H), 3.02-2.97 (m, 1H), 2.45-2.44 (m, 3H), 2.30-2.13 (m, 2H), 2.03 (td, J=4.0, 8.0 Hz, 1H), 1.94-1.86 (m, 1H), 0.96 (d, J=6.4 Hz, 6H).

### 4-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)butanoic acid (Intermediate O)

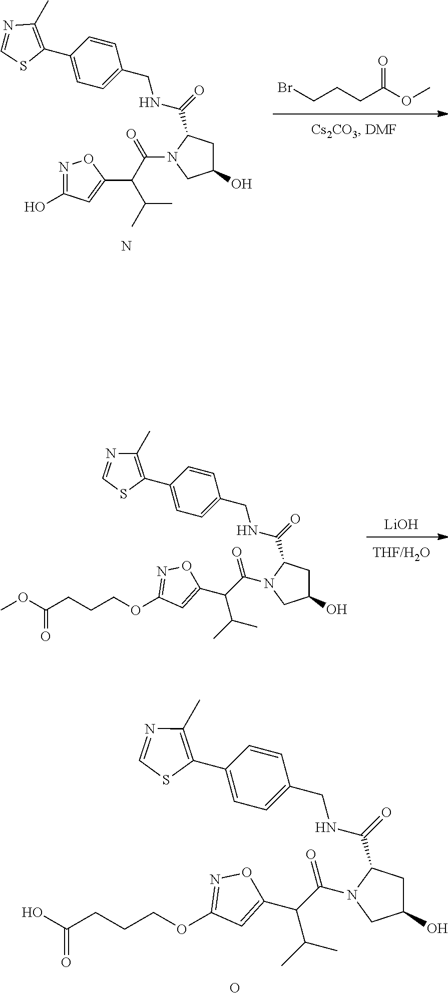

### Step 1—Methyl 4-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)butanoate

To a solution of (2S,4R)-4-hydroxy-1-(2-(3-hydroxyisoxazol-5-yl)-3-methylbutanoyl)-N-(4-(4-methylthiazol-5-yl)benzyl)pyrrolidine-2-carboxamide (1.00 g, 2.06 mmol, Intermediate N), methyl 4-bromobutanoate (448 mg, 2.48 mmol) and Cs₂CO₃ (1.34 g, 4.13 mmol) in DMF (10 mL) was stirred at 25° C., for 12 h. On completion, the reaction mixture was quenched with NH₄Cl (7 mL) and extracted with EtOAc (10 mL×2). The combined organic layers were washed with brine (7 mL×3), dried over Na₂SO₄ and evaporated. The residue was purified by column chromatography (SiO₂, Petroleum ether/Ethyl acetate=3:1˜0:1 (0.1% TEA) to give the title compound (580 mg, 48.07% yield) as a white solid. ¹H NMR (400 MHZ, DMSO-d₆) δ 9.03-8.90 (m, 1H), 8.57-8.43 (m, 1H), 7.51-7.27 (m, 4H), 6.06 (d, J=13.2 Hz, 1H), 5.20-5.06 (m, 1H), 4.46-4.25 (m, 4H), 4.19-4.07 (m, 2H), 3.81-3.70 (m, 1H), 3.66 (d, J=9.6 Hz, 1H), 3.62-3.53 (m, 4H), 2.45 (s, 3H), 2.43-2.41 (m, 1H), 2.41-2.36 (m, 1H), 2.33 (br s, 1H), 2.30-2.21 (m, 1H), 2.09-2.01 (m, 1H), 1.96-1.89 (m, 2H), 1.17 (t, J=7.1 Hz, 1H), 0.98-0.92 (m, 3H), 0.81 (br dd, J=6.8, 13.2 Hz, 3H).

### Step 2—4-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)butanoic acid

To a solution of methyl methyl 4-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)butanoate (530 mg, 907 umol) in THF (5 mL) and H₂O (1.25 mL) was added LiOH (43.4 mg, 1.81 mmol), then the mixture was stirred at 20° C., for 5 h. On completion, the reaction mixture was pH of the aqueous phase was adjusted to 4˜5 by addition 2 M HCl and extracted with EtOAc (10 mL×2). The combined organic layers were washed with brine (7 mL×3), dried over Na₂SO₄ and evaporated to give the title compound (450 mg) as a white solid. ¹H NMR (400 MHZ, DMSO-d₆) δ 12.31-11.92 (m, 1H), 9.03-8.95 (m, 1H), 8.55-8.40 (m, 1H), 7.45-7.32 (m, 4H), 6.10-6.02 (m, 1H), 5.20-5.04 (m, 1H), 4.38-4.27 (m, 4H), 4.16-4.10 (m, 2H), 3.80-3.72 (m, 2H), 2.45 (s, 3H), 2.43 (br s, 1H), 2.37-2.29 (m, 3H), 2.03 (dt, J=3.2, 8.4 Hz, 1H), 1.91 (br t, J=7.2 Hz, 2H), 1.17 (t, J=7.2 Hz, 1H), 0.98-0.93 (m, 3H), 0.84-0.77 (m, 3H).

### 7-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)heptanoic acid (Intermediate P)

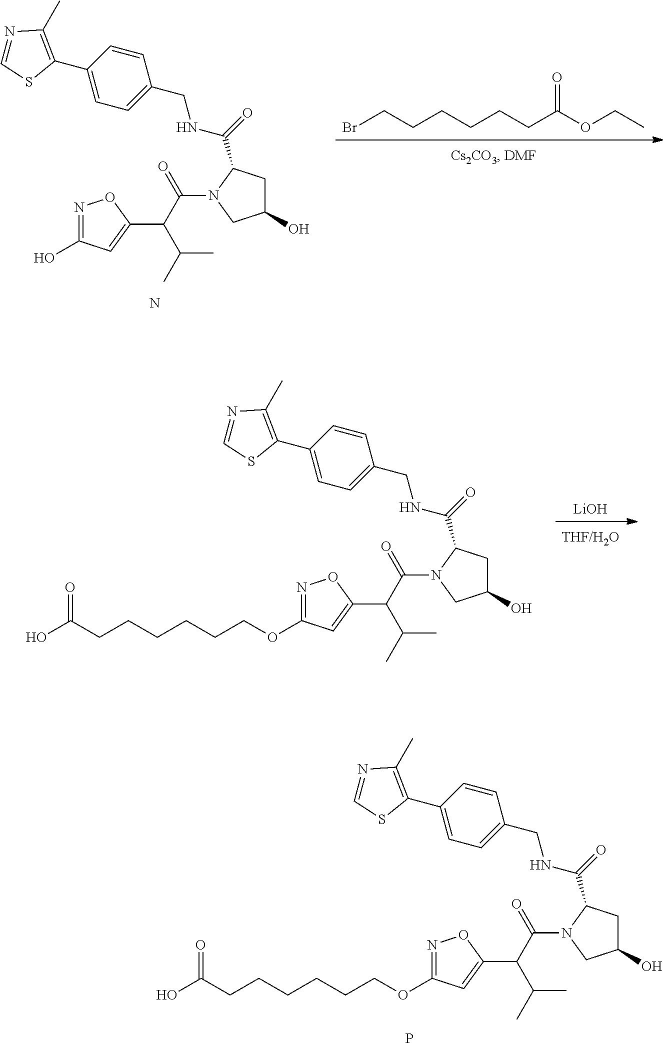

### Step 1—Ethyl 7-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)heptanoate

To a solution of (2S,4R)-4-hydroxy-1-(2-(3-hydroxyisoxazol-5-yl)-3-methylbutanoyl)-N-(4-(4-methylthiazol-5-yl)benzyl)pyrrolidine-2-carboxamide (1.00 g, 2.06 mmol, Intermediate N) and ethyl 7-bromoheptanoate (587 mg, 2.48 mmol, CAS #29823-18-5) in DMF (10 mL) was added to Cs₂CO₃ (1.34 g, 4.13 mmol), then the mixture was stirred at 25° C., for 12 h. On completion, the reaction mixture was quenched with sat. NH₄Cl (30 mL), and then extracted with EtOAc (10 mL×3). The combined organic layers were washed with brine (30 mL×3), dried over Na₂SO₄, filtered and concentrated under reduced pressure to give a residue. The residue was purified by column chromatography (SiO₂, Petroleum ether/Ethyl acetate=1/1 to 0/1) to give the title compound (850 mg, 63% yield) as a white oil. ¹H NMR (400 MHz, DMSO-d₆) δ=8.99 (s, 1H), 8.55-8.43 (m, 1H), 7.45-7.30 (m, 4H), 6.05 (d, J=12.4 Hz, 1H), 5.75 (s, 1H), 5.12 (dd, J=3.6, 6.4 Hz, 1H), 4.47-4.20 (m, 5H), 4.12 (t, J=6.4 Hz, 1H), 4.08-3.98 (m, 4H), 3.79-3.72 (m, 1H), 3.66 (d, J=9.8 Hz, 1H), 3.61-3.53 (m, 1H), 3.49-3.41 (m, 2H), 2.45 (s, 3H), 1.90 (ddd, J=3.6, 8.4, 12.4 Hz, 1H), 1.74-1.59 (m, 3H), 1.56-1.45 (m, 2H), 1.21-1.11 (m, 4H), 1.00-0.92 (m, 3H), 0.81 (dd, J=6.8, 14.0 Hz, 3H).

### Step 2 7-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)heptanoic acid

To a solution of ethyl 7-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)heptanoate (850 mg, 1.33 mmol) in THF (8.5 mL) and H₂O (1.7 mL) was added to LiOH·H₂O (111 mg, 2.65 mmol), then the mixture was stirred at 25° C., for 12 h. On completion, the reaction mixture was acidified to pH to 4˜5 with the addition of HCl (2.5M), then extracted with DCM (10 mL×3). The combined organic layers were washed with brine (10 mL×3), dried over Na₂SO₄, filtered and concentrated under reduced pressure to give the title compound (650 mg) as a yellow oil. LC-MS (ESI+) m/z 613.2 (M+H)⁺.

Tert-butyl methyl (prop-2-yn-1-yl) carbamate (Intermediate Q)

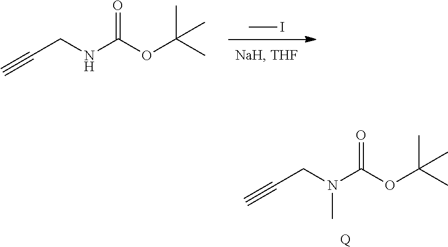

To a solution of tert-butyl prop-2-yn-1-ylcarbamate (50 g, 322 mmol) in THF (500 mL) was added NaH (15.5 g, 387 mmol, 60% dispersion in mineral oil) at 0° C., the mixture was stirred at 0° C., for 0.5 h. Then the iodomethane (91.5 g, 644 mmol, 40.1 mL) was added dropwise, and the reaction mixture was stirred at 25° C., for 12 h under N₂ atmosphere. On completion, the reaction mixture was quenched with H₂O (500 mL) at 20° C., and extracted with EtOAc (200 mL×3). The combined organic layers were washed with brine (200 mL×2), dried over Na₂SO₄, filtered and concentrated under reduced pressure to give the title compound (60 g) as brown oil. ¹H NMR (400 MHZ, DMSO-d₆) δ=3.99 (d, J=2.0 Hz, 2H), 3.18 (s, 1H), 2.80 (br s, 3H), 1.40 (s, 9H).

### Tert-butyl (3-(3-fluoro-4-hydroxyphenyl)prop-2-yn-1-yl)(methyl)carbamate (Intermediate R)

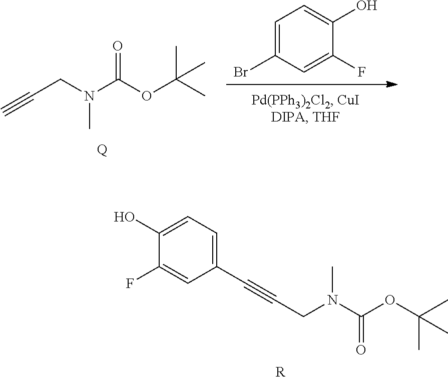

A mixture of 4-bromo-2-fluoro-phenol (2 g, 10.5 mmol), tert-butyl N-methyl-N-prop-2-ynyl-carbamate (3.54 g, 20.9 mmol, Intermediate Q), Pd(PPh₃)₂Cl₂ (367 mg, 523 umol), CuI (99.7 mg, 523 umol) and DIPA (7.42 g, 73.3 mmol, 10.4 mL) in THF (130 mL) was degassed and purged with N₂ three times, and then the mixture was stirred at 60° C., for 12 h under N₂ atmosphere. On completion, the reaction mixture was quenched with H₂O (20 mL) at 20° C., and extracted with EtOAc (20 mL×3). The combined organic layers were washed with brine (20 mL×2), dried over Na₂SO₄, filtered and concentrated under reduced pressure to give a residue. The residue was purified by prep-HPLC (FA condition) to give the title compound (200 mg, 7% yield) as yellow solid. ¹H NMR (400 MHZ, DMSO-d₆) δ=10.32 (br s, 1H), 7.22 (dd, J=1.6, 11.6 Hz, 1H), 7.08 (dd, J=1.2, 8.4 Hz, 1H), 6.92 (t, J=8.8 Hz, 1H), 4.21 (s, 2H), 2.85 (br s, 3H), 1.41 (s, 9H).

### Methyl 2-(8-(benzo[d]thiazol-2-ylcarbamoyl)-3,4-dihydroisoquinolin-2 (1H)-yl)-5-(3-(tosyloxy)propyl)thiazole-4-carboxylate (Intermediate T)

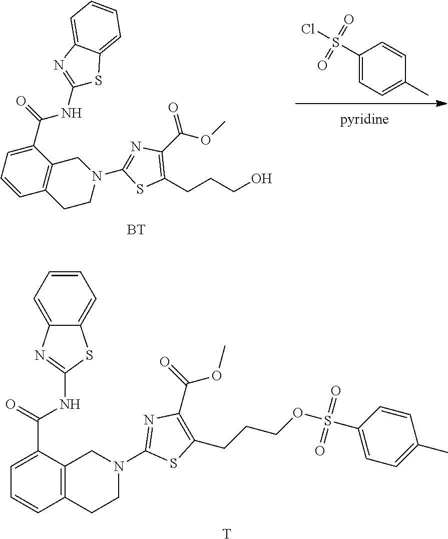

To a solution of methyl 2-(8-(benzo[d]thiazol-2-ylcarbamoyl)-3,4-dihydroisoquinolin-2 (1H)-yl)-5-(3-hydroxypropyl)thiazole-4-carboxylate (200 mg, 393 umol, Intermediate BT) in pyridine (2.5 mL) was added 4-methylbenzenesulfonyl chloride (150 mg, 786 umol) at 0° C., then the mixture was stirred at 25° C., for 2 h. On completion, the reaction mixture was quenched with H₂O (5 mL) at 20° C., then extracted with EtOAc (2.5 mL×3). The combined organic layers were washed with brine (2.5 mL×2), dried over Na₂SO₄, filtered and concentrated under reduced pressure to give a residue. The residue was purified by column chromatography (SiO₂, Petroleum ether/Ethyl acetate=5/1 to 1/1) to give the title compound (80 mg, 30.7% yield) as brown gum. LC-MS (ESI+) m/z 663.1 (M+H)⁺.

### Methyl 2-(8-(benzo[d]thiazol-2-ylcarbamoyl)-3,4-dihydroisoquinolin-2 (1H)-yl)-5-(3-(2-fluoro-4-(3-(methylamino)prop-1-yn-1-yl)phenoxy)propyl)thiazole-4-carboxylate (Intermediate U)

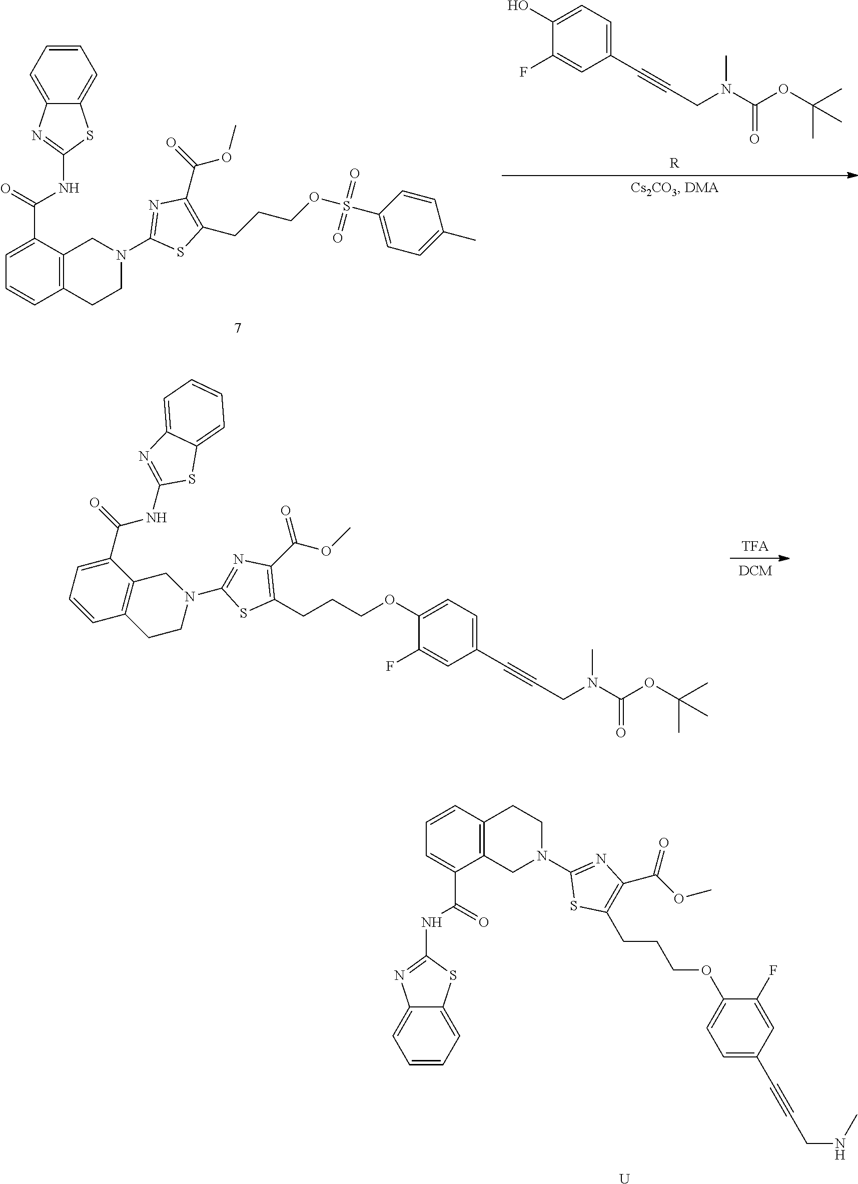

### Step 1—Methyl 2-(8-(benzo[d]thiazol-2-ylcarbamoyl)-3,4-dihydroisoquinolin-2 (1H)-yl)-5-(3-(4-(3-((tert-butoxycarbonyl)(methyl)amino)prop-1-yn-1-yl)-2-fluorophenoxy)propyl)thiazole-4-carboxylate

To a solution of tert-butyl (3-(3-fluoro-4-hydroxyphenyl)prop-2-yn-1-yl)(methyl)carbamate (50.6 mg, 181 umol, Intermediate R) and methyl 2-(8-(benzo[d]thiazol-2-ylcarbamoyl)-3,4-dihydroisoquinolin-2 (1H)-yl)-5-(3-(tosyloxy)propyl)thiazole-4-carboxylate (80 mg, 121 umol, Intermediate T) in DMA (2 mL) was added Cs₂CO₃ (59.0 mg, 181 umol), then the mixture was stirred at 25° C., for 12 h. On completion, filtered and concentrated under reduced pressure to give the title compound (80 mg) as yellow solid. LC-MS (ESI+) m/z 770.3 (M+H)⁺.

### Step 2—Methyl 2-(8-(benzo[d]thiazol-2-ylcarbamoyl)-3,4-dihydroisoquinolin-2 (1H)-yl)-5-(3-(2-fluoro-4-(3-(methylamino)prop-1-yn-1-yl)phenoxy)propyl)thiazole-4-carboxylate

A solution of methyl 2-(8-(benzo[d]thiazol-2-ylcarbamoyl)-3,4-dihydroisoquinolin-2 (1H)-yl)-5-(3-(4-(3-((tert-butoxycarbonyl)(methyl)amino)prop-1-yn-1-yl)-2-fluorophenoxy)propyl)thiazole-4-carboxylate (50 mg, 64.9 umol) in DCM (2 mL) was added TFA (308 mg, 2.70 mmol, 0.2 mL), then the mixture was stirred at 25° C., for 1 h. On completion, filtered and concentrated under reduced pressure to give the title compound (50 mg, crude) as brown gum. LC-MS (ESI+) m/z 670.1 (M+H)⁺.

### 9-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]nonanoic acid (Intermediate V)

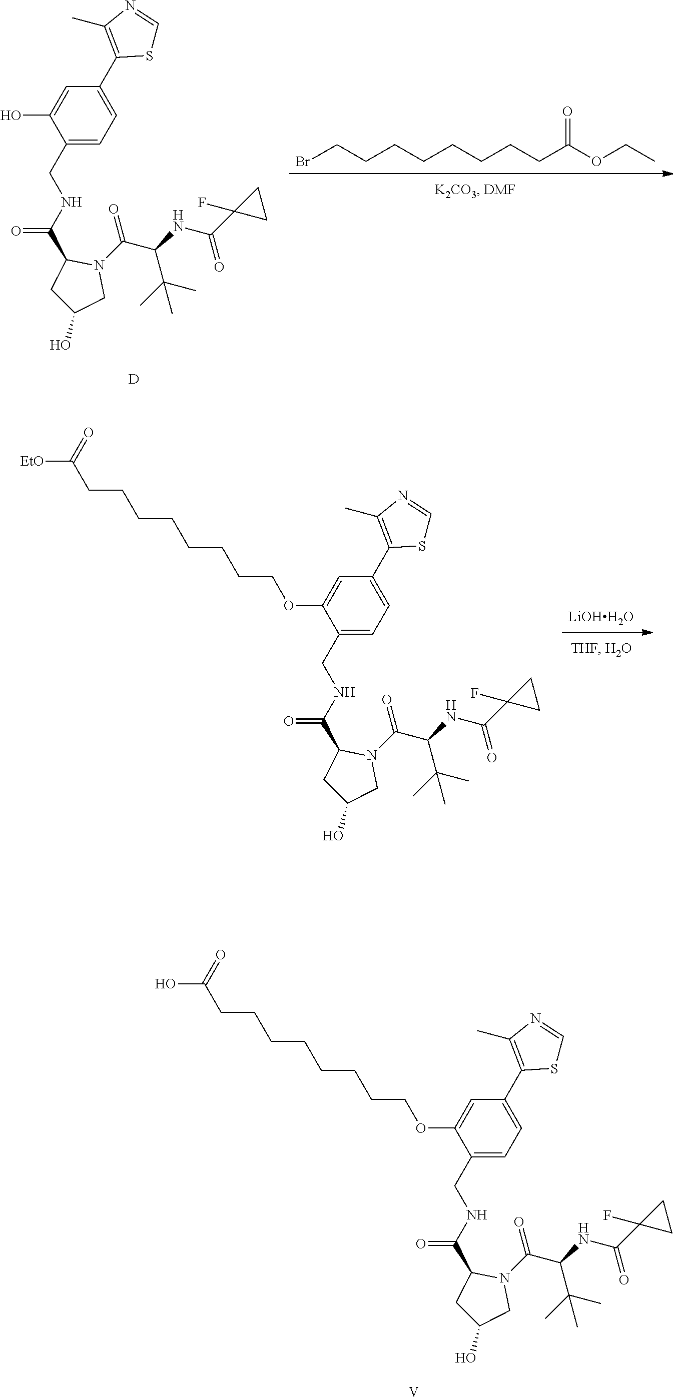

### Step 1—Ethyl 9-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)nonanoate

A mixture of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (656 mg, 1.23 mmol, Intermediate D), ethyl 9-bromononanoate (490 mg, 1.85 mmol, CAS #28598-81-4), K₂CO₃ (511 mg, 3.70 mmol) in DMF (5 mL), and then the mixture was stirred at 70° C., for 12 h. On completion, the reaction mixture was quenched with ice water (5 mL) at 20° C., and extracted with EtOAc (4 mL×2). The combined organic layers were washed with brine (4 mL×2), dried over Na₂SO₄, filtered and concentrated under reduced pressure to give a residue. The residue was purified by column chromatography (SiO₂, Petroleum ether/Ethyl acetate=1/1 to 0/1) to give the title compound (0.835 g, 95% yield) as a yellow gum. ¹H NMR (400 MHZ, DMSO-d₆) δ=8.98 (s, 1H), 8.53-8.41 (m, 1H), 7.40 (d, J=8.0 Hz, 1H), 7.34-7.24 (m, 1H), 6.99 (d, J=1.2 Hz, 1H), 6.94 (dd, J=1.6, 7.8 Hz, 1H), 5.16 (d, J=3.6 Hz, 1H), 4.59 (d, J=9.2 Hz, 1H), 4.51 (t, J=8.0 Hz, 1H), 4.35 (br s, 1H), 4.27 (br d, J=6.0 Hz, 1H), 4.21 (br d, J=5.6 Hz, 1H), 3.70-3.57 (m, 2H), 2.45 (s, 4H), 2.26 (t, J=7.6 Hz, 2H), 2.07 (br d, J=8.0 Hz, 1H), 1.96-1.88 (m, 1H), 1.80-1.70 (m, 2H), 1.52 (br t, J=6.8 Hz, 3H), 1.47-1.41 (m, 2H), 1.41-1.37 (m, 2H), 1.36-1.20 (m, 11H), 0.96 (s, 11H).

### Step 2—9-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)nonanoic acid

To a solution of ethyl 9-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)nonanoate (835 mg, 1.16 mmol) in THF (8.5 mL) and H₂O (2.2 mL) was added LiOH—H₂O (195 mg, 4.66 mmol). The mixture was stirred at 25° C., for 12 h. On completion, the reaction mixture was quenched with ice water (12 mL) at 20° C., and extracted with EtOAc (5 mL×2). Then the aqueous layer was adjusted to pH=3˜ 4 and extracted with EtOAc (5 mL×2). The organic layer was washed with brine (5 mL×2), dried over Na₂SO₄, filtered and concentrated under reduced pressure to give the title compound (549 mg, 68% yield) as a yellow gum. ¹H NMR (400 MHZ, DMSO-d₆) δ=11.95 (br s, 1H), 8.98 (s, 1H), 8.48 (br t, J=5.6 Hz, 1H), 7.40 (br d, J=8.0 Hz, 1H), 7.29 (br d, J=7.6 Hz, 1H), 6.99 (s, 1H), 6.94 (br d, J=8.0 Hz, 1H), 5.17 (br d, J=3.2 Hz, 1H), 4.60 (br d, J=9.2 Hz, 1H), 4.51 (br t, J=8.4 Hz, 1H), 4.35 (br s, 1H), 4.27 (br d, J=6.0 Hz, 1H), 4.21 (br d, J=5.6 Hz, 1H), 4.04 (br t, J=6.4 Hz, 2H), 3.70-3.55 (m, 2H), 2.45 (s, 3H), 2.19 (br t, J=7.2 Hz, 2H), 2.13-2.04 (m, 1H), 1.94-1.87 (m, 2H), 1.78-1.71 (m, 2H), 1.54-1.43 (m, 4H), 1.30 (br s, 6H), 1.24-1.16 (m, 3H), 0.96 (s, 9H).

### 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)butanoic acid (Intermediate W)

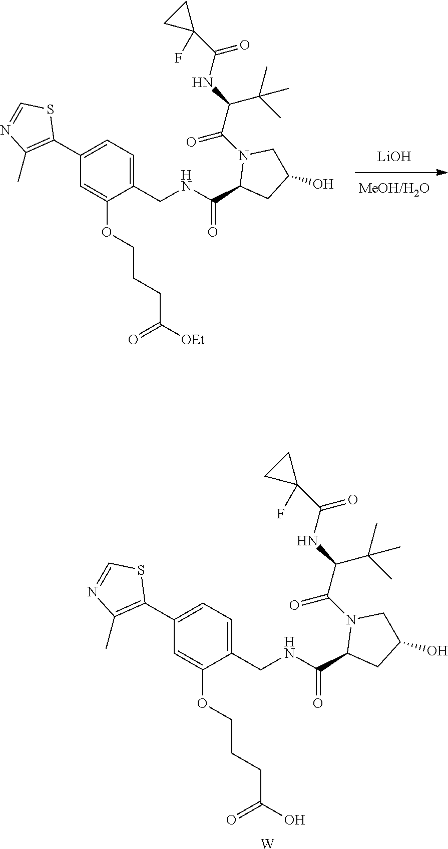

To a solution of methyl 4-[2-[[[(2S,4R)-1-[(2S)-2-[(1-fluorocyclopropanecarbonyl)amino]-3,3-dimethyl-butanoyl]-4-hydroxypyrrolidine-2-carbonyl]amino]methyl]-5-(4-methylthiazol-5-yl)phenoxy]butanoate (500 mg, 773 umol, synthesized via Step 1 of Intermediate E) in MeOH (5 mL) and H₂O (1 mL) was added LiOH·H₂O (162 mg, 3.87 mmol), then the reaction was then stirred at 25° C., for 2 hours. On completion, the reaction mixture was concentrated in vacuo, and diluted with water (2 ml). The mixture was then acidified with HCl (4 N) until the pH=6, then the mixture was extracted with EtOAc (5 ml×2). The combined organic layers were washed with saturated brine, dried over Na₂SO₄, filtered and concentrated in vacuo to give the title compound (440 mg, 92% yield) as a white solid. ¹H NMR (400 MHZ, DMSO-d₆) δ 12.21-12.07 (m, 1H), 9.01-8.95 (m, 1H), 8.50 (br t, J=6.0 Hz, 1H), 7.44-7.38 (m, 1H), 7.29 (br dd, J=2.4, 9.2 Hz, 1H), 7.00 (s, 1H), 6.95 (d, J=7.6 Hz, 1H), 5.25-5.09 (m, 1H), 4.60 (d, J=9.2 Hz, 1H), 4.52 (t, J=8.2 Hz, 1H), 4.35 (br s, 1H), 4.25 (br s, 2H), 4.07 (br t, J=6.0 Hz, 2H), 3.68-3.59 (m, 2H), 2.45 (s, 3H), 2.44-2.41 (m, 2H), 2.13-2.05 (m, 1H), 2.02 (br s, 1H), 1.98-1.88 (m, 2H), 1.41-1.32 (m, 2H), 1.25-1.19 (m, 2H), 0.96 (s, 9H)

### 6-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)hexanoic acid (Intermediate X)

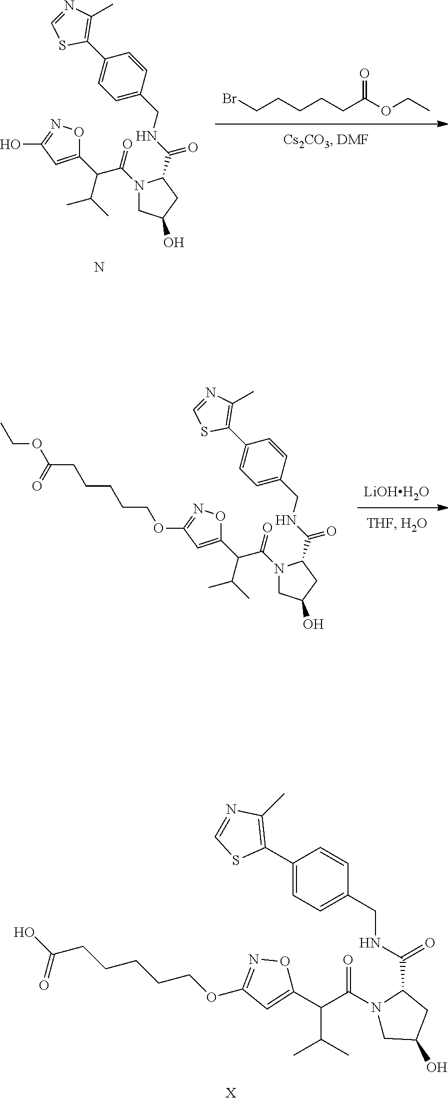

### Step 1 Ethyl-6-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)hexanoate

A mixture of (2S,4R)-4-hydroxy-1-(2-(3-hydroxyisoxazol-5-yl)-3-methylbutanoyl)-N-(4-(4-methylthiazol-5-yl)benzyl)pyrrolidine-2-carboxamide (1 g, 2 mmol, Intermediate N), ethyl 6-bromohexanoate (552 mg, 2.48 mmol) and Cs₂CO; (1.34 g, 4.13 mmol) in DMF (10 mL) was stirred at 25° C., for 2 h under N₂ atmosphere. On completion, the reaction mixture was quenched with H₂O (10 mL), and then extracted with EtOAc (10 mL×3). The combined organic layers were washed with brine (10 mL×3), dried over Na₂SO₄, filtered and concentrated under reduced pressure to give a residue. The residue was purified by flash silica gel chromatography (ISCO®; 12 g SepaFlash® Silica Flash Column, Eluent of 90˜100% Ethyl acetate/Petroleum ethergradient @ 50 mL/min) to give the title compound (790 mg, 59% yield) as white oil. ¹H NMR (400 MHZ, DMSO-d₆) δ=8.98 (d, J=1.6 Hz, 1H), 8.54-8.41 (m, 1H), 7.46-7.40 (m, 2H), 7.40-7.33 (m, 2H), 6.05 (d, J=11.2 Hz, 1H), 5.11 (dd, J=4.0, 6.4 Hz, 1H), 4.38-4.30 (m, 3H), 4.12 (t, J=6.4 Hz, 1H), 4.04-4.01 (m, 2H), 3.79-3.73 (m, 1H), 2.45 (s, 3H), 2.32-2.19 (m, 4H), 2.03 (dt, J=4.0, 8.2 Hz, 1H), 1.99 (s, 1H), 1.91 (tt, J=4.2, 8.2 Hz, 1H), 1.73-1.67 (m, 1H), 1.64 (br dd, J=6.8, 14.4 Hz, 1H), 1.58-1.51 (m, 2H), 1.40-1.31 (m, 2H), 1.20-1.13 (m, 5H), 0.98-0.93 (m, 3H), 0.81 (dd, J=6.8, 14.0 Hz, 3H).

### Step 2 6-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)hexanoic acid

The mixture of ethyl ethyl 6-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)hexanoate (790 mg, 1.3 mmol) LiOH·H₂O (106 mg, 2.52 mmol) in THF (8 mL) and H₂O (2 mL) was stirred at 20° C., for 12 h. On completion, the reaction mixture was quenched with HCl and the pH was adjusted to 4˜5. Then the mixture was addition H₂O (10 mL), and then extracted with EtOAc (10 mL×3). The combined organic layers were washed with brine (10 mL×2), dried over Na₂SO₄, filtered and concentrated under reduced pressure to give the title compound (660 mg) as white solid. ¹H NMR (400 MHZ, DMSO-d₆) δ=12.05 (br s, 1H), 9.04 (d, J=2.0 Hz, 1H), 8.61-8.50 (m, 1H), 7.52-7.38 (m, 4H), 6.14-6.09 (m, 1H), 5.19 (dd, J=3.6, 6.1 Hz, 1H), 4.52-4.33 (m, 4H), 4.19 (t, J=6.4 Hz, 1H), 4.14-4.08 (m, 1H), 3.85-3.79 (m, 1H), 3.72 (d, J=9.6 Hz, 1H), 3.63-3.61 (m, 1H), 3.51 (brd, J=11.6 Hz, 2H), 2.51 (s, 2H), 2.31-2.22 (m, 3H), 2.15-2.06 (m, 1H), 2.00-1.93 (m, 1H), 1.79-1.67 (m, 2H), 1.62-1.53 (m, 2H), 1.47-1.37 (m, 2H), 1.05-0.98 (m, 3H), 0.87 (dd, J=6.8, 13.8 Hz, 3H).

### 9-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)nonanoic acid (Intermediate Y)

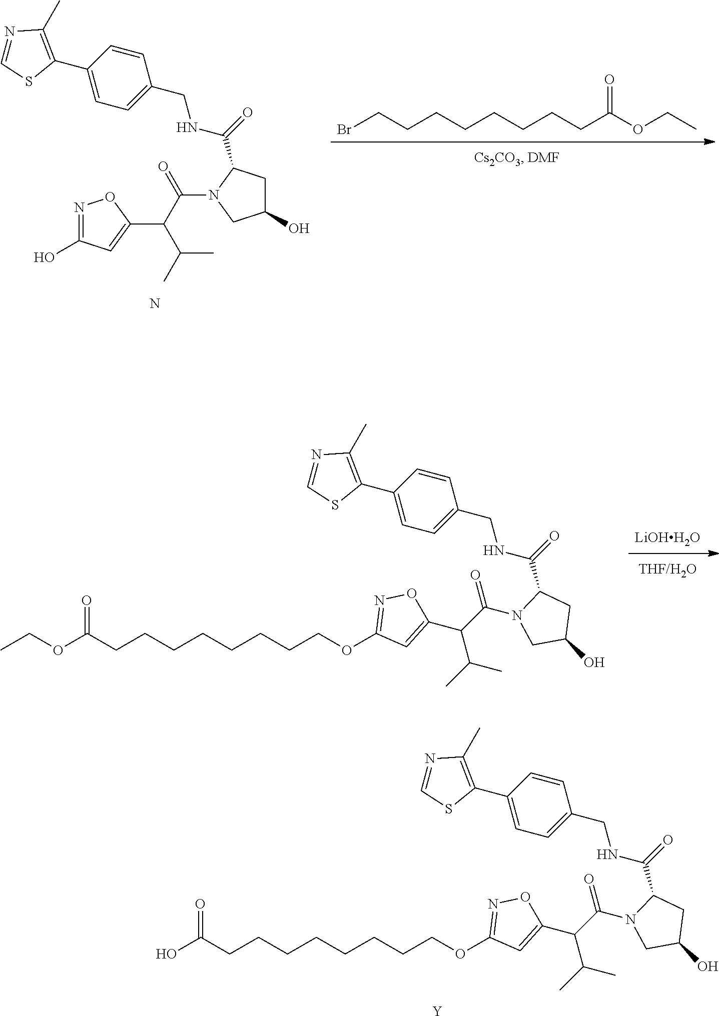

### Step 1—Ethyl 9-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)nonanoate

A solution of (2S,4R)-4-hydroxy-1-(2-(3-hydroxyisoxazol-5-yl)-3-methylbutanoyl)-N-(4-(4-methylthiazol-5-yl)benzyl)pyrrolidine-2-carboxamide (1.00 g, 2.06 mmol, Intermediate N), ethyl 9-bromononanoate (657 mg, 2.48 mmol, CAS #28598-81-4) and Cs₂CO₃ (1.34 g, 4.13 mmol) in DMF (10 mL) was stirred at 25° C., for 4 h. On completion, the reaction mixture was diluted with water (20 mL), and extracted with EtOAc (25 mL×3). The combined organic layers were washed with brine (10 mL×4), dried over Na₂SO₄ and evaporated. The residue was purified by column chromatography (SiO₂, Petroleum ether/Ethyl acetate=1/3 to 0/1) to afford the title compound (760 mg, 55% yield) as a yellow oil. LC-MS (ESI+) m/z 669.3 (M+H)⁺.

### Step 2 9-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)nonanoic acid

To a solution of ethyl 9-((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)nonanoate (910 mg, 1.36 mmol) in THF (8 mL) and H₂O (1.6 mL) was added LiOH·H₂O (228 mg, 5.44 mmol). The mixture was stirred at 25° C., for 24 h. On completion, the reaction mixture was diluted with water (5 mL) and the pH was adjusted to 4˜3 by addition of 2 M HCl. Then the mixture was extracted with EtOAc (20 mL×3). The combined organic layers were washed with brine (10 mL×3), dried over Na₂SO₄ and evaporated to afford the title compound (870 mg) as a colorless oil. ¹H NMR (400 MHZ, DMSO-d₆) δ=11.95 (br s, 1H), 8.98 (d, J=2.0 Hz, 1H), 8.54-8.39 (m, 1H), 7.48-7.30 (m, 5H), 6.08-6.03 (m, 1H), 5.12 (dd, J=3.6, 6.4 Hz, 1H), 4.47-4.21 (m, 5H), 4.14-4.05 (m, 2H), 3.80-3.73 (m, 1H), 3.61-3.55 (m, 1H), 2.45 (s, 3H), 2.28-2.22 (m, 1H), 2.21-2.14 (m, 3H), 2.07-2.01 (m, 1H), 1.91-1.90 (m, 1H), 1.71-1.60 (m, 2H), 1.50-1.45 (m, 2H), 1.28-1.23 (m, 6H), 0.98-0.93 (m, 3H), 0.81 (dd, J=6.8, 14.4 Hz, 3H).

### Methyl 2-chloro-5-(3-(2-fluoro-6-iodophenoxy)propyl)thiazole-4-carboxylate (Intermediate Z)

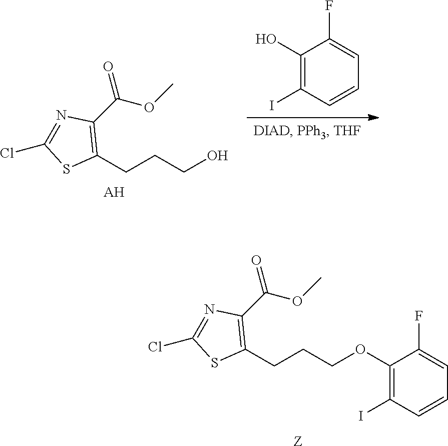

To a solution of methyl 2-chloro-5-(3-hydroxypropyl)thiazole-4-carboxylate (2 g, 8.49 mmol, Intermediate AH) and 2-fluoro-6-iodo-phenol (3.03 g, 12.7 mmol) and PPh; (3.34 g, 12.7 mmol) in THF (45 mL) was a solution of DIAD (2.57 g, 12.7 mmol) in THF (20 mL) at 0° C., under nitrogen flow. Then the reaction was stirred at 20° C., for 10 h under nitrogen atmosphere. On completion, the reaction was filtered to get the filtrate and concentrated to give a residue. The residue was purified by column chromatography on silica gel (eluted with petroleum ether:ethyl acetate=100:1 to 100:10) to give the title compound (3 g, 78% yield) as a colorless oil. ¹H NMR (400 MHZ, CDCl₃) δ=7.54 (td, J=1.2, 8.0 Hz, 1H), 7.09 (ddd, J=1.6, 8.2, 10.8 Hz, 1H), 6.80 (dt, J=5.2, 8.0 Hz, 1H), 4.22-4.14 (m, 2H), 3.94 (s, 3H), 3.60-3.43 (m, 2H), 2.34-2.16 (m, 2H).

### N-(benzo[d]thiazol-2-yl)-1,2,3,4-tetrahydroisoquinoline-8-carboxamide (Intermediate AA)

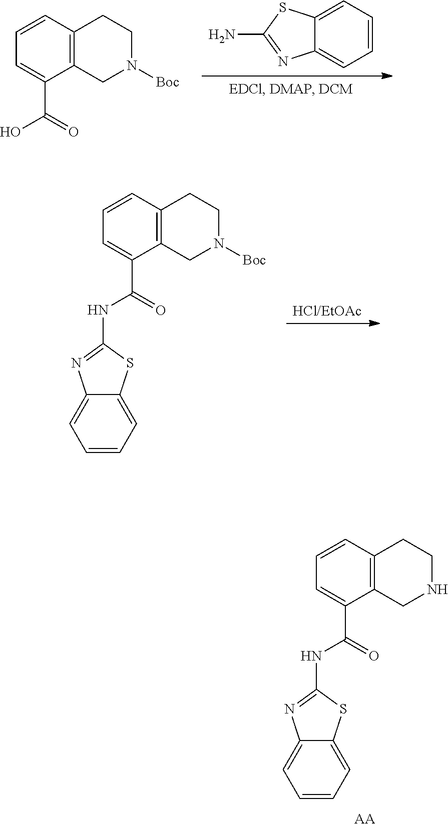

### Step 1—Tert-butyl 8-(benzo[d]thiazol-2-ylcarbamoyl)-3,4-dihydroisoquinoline-2 (1H)-carboxylate

To a solution of 2-(tert-butoxycarbonyl)-1,2,3,4-tetrahydroisoquinoline-8-carboxylic acid (50.0 g, 180 mmol, CAS #878798-87-9) and benzo[d]thiazol-2-amine (40.6 g, 270 mmol, CAS #136-95-8) in DCM (600 mL) was added DMAP (44.0 g, 360 mmol) and EDCI (69.1 g, 360 mmol). The mixture was stirred at 20° C., for 3 hrs. On completion, the reaction mixture was diluted with DCM (300 mL). The combined organic layers were washed with 5% aq. HCl (300 mL×3), water (200 mL) and brine (200 mL), then dried over Na₂SO₄, filtered and concentrated under reduced pressure to give a residue. The crude product was purified by column chromatography (SiO₂, Petroleum ether/Ethyl acetate=200/1 to 0/1) to give the title compound (85.53 g, 83% yield) as a white solid. LC-MS (ESI+) m/z 410.0 (M+H)⁺. ¹H NMR (400 MHz, CDCl₃) δ 11.5 (brs, 1H), 7.74 (d, J=7.2 Hz, 1H), 7.40 (d, J=7.2 Hz, 1H), 7.21-7.12 (m, 4H), 7.02-6.96 (m, 2H), 4.84 (m, 2H), 3.57 (brs, 2H), 2.81 (brs, 2H), 1.39 (m, 9H).

### Step 2—N-(benzo[d]thiazol-2-yl)-1,2,3,4-tetrahydroisoquinoline-8-carboxamide4

To a solution of Tert-butyl 8-(benzo[d]thiazol-2-ylcarbamoyl)-3,4-dihydroisoquinoline-2 (1H)-carboxylate (80.0 g, 195 mmol) in HCl/EtOAc (4 M, 1200 mL). The mixture was then stirred at 20° C., for 1 hr. On completion, the reaction mixture was filtered and the mixture was concentrated and the residue was dried in vacuo to give the title compound (73.4 g, 96% yield, 2HCI) as a white solid. ¹H NMR: EC2783-4-PIC (400 MHz, DMSO) ò 9.55 (s, 1H), 8.02 (d, J=7.2 Hz, 1H), 7.79-7.73 (m, 2H), 7.47-7.44 (m, 3H), 7.35 (t, J=6.8 Hz, 1H), 4.47 (s, 2H), 3.36 (s, 2H), 3.10 (t, J=6.0 Hz, 2H), 2.50 (s, 9H).

### Methyl 2-(8-(benzo[d]thiazol-2-ylcarbamoyl)-3,4-dihydroisoquinolin-2 (1H)-yl)-5-(3-(2-fluoro-6-(3-(methylamino)prop-1-yn-1-yl)phenoxy)propyl)thiazole-4-carboxylate (Intermediate AB)

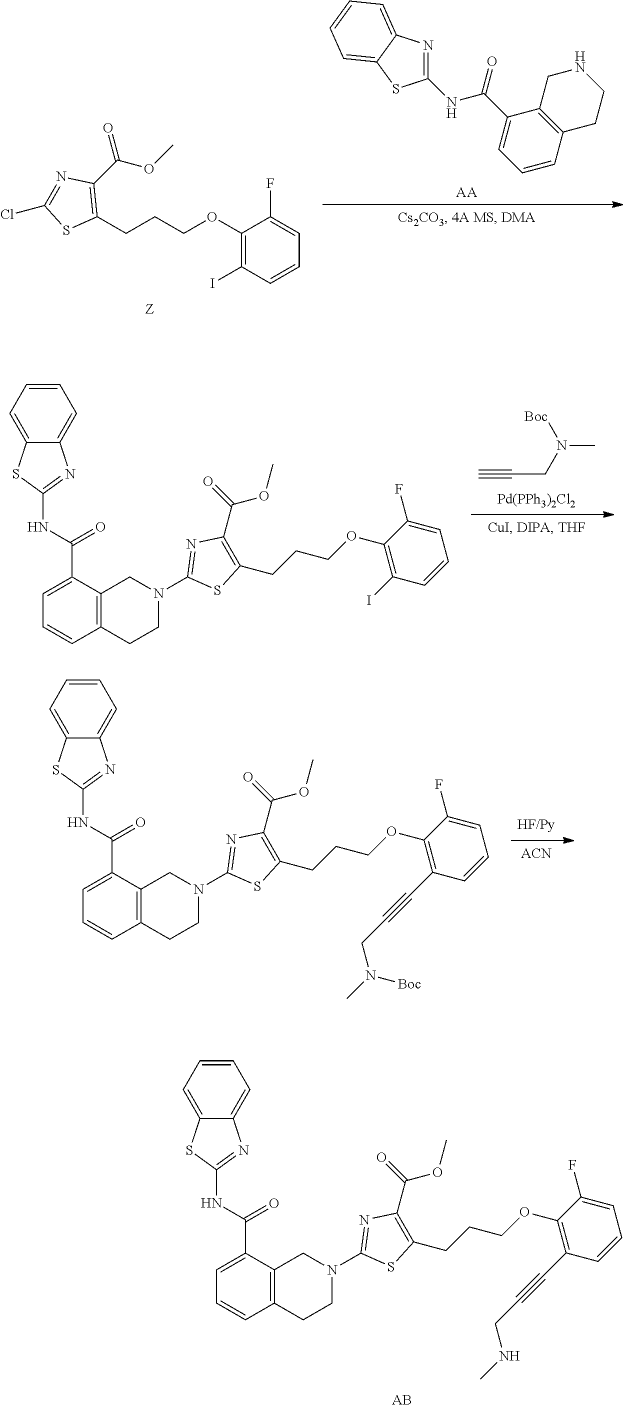

### Step 1—Methyl 2-(8-(benzo[d]thiazol-2-ylcarbamoyl)-3,4-dihydroisoquinolin-2 (1H)-yl)-5-(3-(2-fluoro-6-iodophenoxy)propyl)thiazole-4-carboxylate

To a solution of N-(benzo[d]thiazol-2-yl)-1,2,3,4-tetrahydroisoquinoline-8-carboxamide (2.04 g, 6.58 mmol, Intermediate AA) and methyl 2-chloro-5-(3-(2-fluoro-6-iodophenoxy)propyl)thiazole-4-carboxylate (3 g, 7 mmol, Intermediate Z) in DMA (70 mL) and was added Cs₂CO₃ (6.44 g, 19.7 mmol) and 4 Å molecular sieves (3.00 g, 30.0 mmol) at 20° C., under nitrogen flow. Then the reaction was stirred at 100° C., for 10 h under nitrogen atmosphere. On completion, the reaction was filtered to get the filtrate and concentrated to give a residue. The residue was purified by column chromatography on silica gel (eluted with petroleum ether:tetrahydrofuran=100:1 to 100:50) to give the title compound (2.2 g, 46% yield) as a yellow solid. ¹H NMR (400 MHZ, CDCl₃) δ 7.89-7.79 (m, 1H), 7.59 (d, J=7.2 Hz, 1H), 7.55-7.44 (m, 3H), 7.37-7.30 (m, 3H), 7.26-7.16 (m, 1H), 7.03 (ddd, J=1.6, 8.2, 10.8 Hz, 1H), 6.80-6.67 (m, 1H), 4.89 (s, 2H), 4.12 (br t, J=5.6 Hz, 2H), 3.80 (s, 3H), 3.75-3.71 (m, 3H), 3.34 (br t, J=7.6 Hz, 2H), 2.19-2.12 (m, 3H).

### Step 2—Methyl 2-(8-(benzo[d]thiazol-2-ylcarbamoyl)-3,4-dihydroisoquinolin-2 (1H)-yl)-5-(3-(2-(3-((tert-butoxycarbonyl)(methyl)amino)prop-1-yn-1-yl)-6-fluorophenoxy)propyl)thiazole-4-carboxylate

To a solution of methyl 2-(8-(benzo[d]thiazol-2-ylcarbamoyl)-3,4-dihydroisoquinolin-2 (1H)-yl)-5-(3-(2-fluoro-6-iodophenoxy)propyl)thiazole-4-carboxylate (232.26 mg, 1.37 mmol) in THF (10 mL) was added Pd(PPh₃)₂Cl₂ (48.1 mg, 68.6 umol), DIPA (486 mg, 4.80 mmol) and CuI (13.0 mg, 68.63 umol) at 20° C., under nitrogen flow. Then the reaction was stirred at 60° C., for 10 h under nitrogen atmosphere. On completion, the reaction was filtered to get the filtrate and concentrated to give a residue. The residue was purified by column chromatography on silica gel (eluted with petroleum ether:tetrahydrofuran=100:1 to 100:50) to give the title compound (400 mg, 76% yield) as a yellow oil. ¹H NMR (400 MHZ, CDCl₃) δ 7.85 (br d, J=7.2 Hz, 1H), 7.56 (br d, J=7.2 Hz, 1H), 7.38-7.26 (m, 4H), 7.22-7.15 (m, 1H), 7.12 (br d, J=7.6 Hz, 1H), 7.08-6.97 (m, 1H), 6.96-6.86 (m, 1H), 4.89 (s, 2H), 4.30 (br s, 2H), 4.20 (br t, J=6.0 Hz, 2H), 3.92-3.83 (m, 3H), 3.81 (s, 2H), 3.31 (br t, J=7.6 Hz, 2H), 3.14 (br d, J=6.4 Hz, 1H), 3.03 (br t, J=5.2 Hz, 2H), 2.96 (s, 3H), 2.14-2.06 (m, 2H), 1.45 (s, 9H).

### Step 3—Methyl 2-(8-(benzo[d]thiazol-2-ylcarbamoyl)-3,4-dihydroisoquinolin-2 (1H)-yl)-5-(3-(2-fluoro-6-(3-(methylamino)prop-1-yn-1-yl)phenoxy)propyl)thiazole-4-carboxylate

To a solution of methyl 2-(8-(benzo[d]thiazol-2-ylcarbamoyl)-3,4-dihydroisoquinolin-2 (1H)-yl)-5-(3-(2-(3-((tert-butoxycarbonyl)(methyl)amino)prop-1-yn-1-yl)-6-fluorophenoxy)propyl)thiazole-4-carboxylate (400 mg, 5 umol) in ACN (8 mL) was added pyridine: hydrofluoride (3.09 g, 31.1 mmol) at 20° C., under nitrogen flow. Then the reaction was stirred at 20° C., for 1 min under nitrogen atmosphere. On completion, the reaction was under lyophilization to give the title compound (800 mg) as a brown solid. LC-MS (ESI+) m/z 670.1 (M+H)⁺. 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid (Intermediate AC)

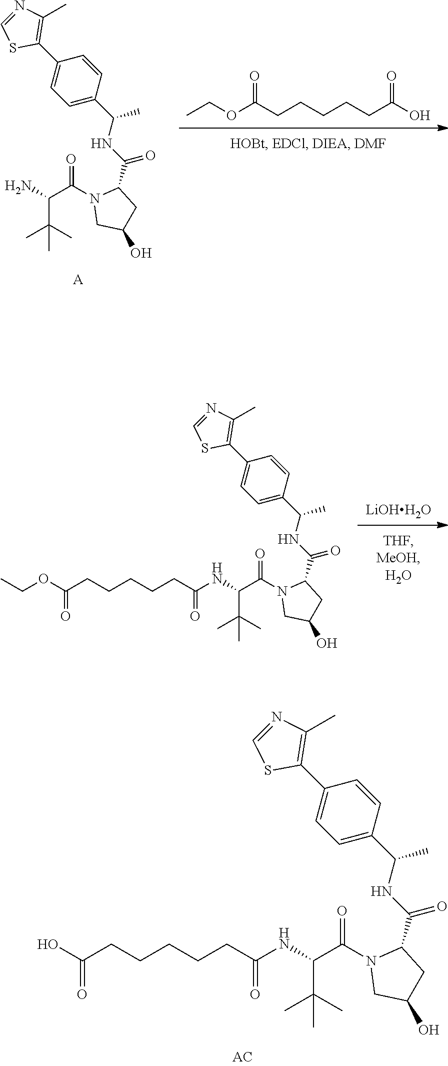

### Step 1—Ethyl 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoate

To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (2.00 g, 3.86 mmol, 2 HCl, Intermediate A), 7-ethoxy-7-oxo-heptanoic acid (1.09 g, 5.80 mmol) and DIEA (2.50 g, 19.3 mmol) in DMF (20 mL) was added HOBt (783 mg, 5.80 mmol), and EDCI (1.11 g, 5.80 mmol). The mixture was stirred at 25° C., for 12 h. On completion, the reaction mixture was diluted with water (50 mL), and extracted with DCM (50 mL×3). The combined organic layers were washed with brine (30 mL×4), dried over Na₂SO₄ and evaporated. The residue was purified by column chromatography (SiO₂, Petroleum ether/Ethyl acetate=0/1 to DCM/MeOH=10:1) to afford the title compound (1.80 g, 76% yield) as a yellow solid. ¹H NMR (400 MHZ, DMSO-d₆) δ=8.98 (s, 1H), 8.36 (d, J=8.0 Hz, 1H), 7.79 (d, J=9.2 Hz, 1H), 7.45-7.41 (m, 2H), 7.40-7.36 (m, 2H), 5.09 (d, J=3.6 Hz, 1H), 4.91 (t, J=7.2 Hz, 1H), 4.51 (d, J=9.2 Hz, 1H), 4.42 (t, J=8.0 Hz, 1H), 4.27 (d, J=2.8 Hz, 1H), 4.11-4.00 (m, 3H), 3.65-3.55 (m, 2H), 3.17 (d, J=5.2 Hz, 2H), 2.45 (s, 3H), 2.25 (t, J=7.6 Hz, 2H), 1.57-1.45 (m, 4H), 1.37 (d, J=7.0 Hz, 3H), 1.28-1.21 (m, 2H), 1.17 (t, J=7.1 Hz, 3H), 0.95-0.90 (m, 9H).

### Step 2—7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid

To a solution of ethyl 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoate (1.80 g, 2.93 mmol) in THF (15 mL), MeOH (3 mL) and H₂O (3 mL) was added LiOH·H₂O (491 mg, 11.7 mmol). The mixture was stirred at 25° C., for 2 h. On completion, the reaction mixture was diluted with water (10 mL) and the pH was adjusted to 4˜3 by addition 2 M HCl. Then the mixture was extracted with EtOAc (20 mL×3). The combined organic layers were washed with brine (10 mL×4), dried over Na₂SO₄ and evaporated to afford the title compound (1.70 g, 99% yield) as a white solid. JH NMR (400 MHZ, DMSO-d₆) δ=11.96 (br d, J=4.0 Hz, 1H), 8.99 (s, 1H), 8.37 (d, J=8.0 Hz, 1H), 7.80 (d, J=9.2 Hz, 1H), 7.45-7.42 (m, 2H), 7.40-7.36 (m, 2H), 5.24-4.99 (m, 1H), 4.92 (t, J=7.2 Hz, 1H), 4.51 (d, J=9.2 Hz, 1H), 4.42 (t, J=8.0 Hz, 1H), 4.28 (br s, 1H), 3.64-3.58 (m, 2H), 3.42 (br s, 2H), 3.17 (s, 2H), 2.18 (t, J=7.4 Hz, 2H), 1.52-1.45 (m, 4H), 1.38 (d, J=7.2 Hz, 3H), 1.28-1.22 (m, 2H), 0.95-0.92 (m, 9H).

### Methyl 2-(8-(benzo[d]thiazol-2-ylcarbamoyl)-3,4-dihydroisoquinolin-2 (1H)-yl)-5-(3-(2-fluoro-6-(3-(methylamino)propyl)phenoxy)propyl)thiazole-4-carboxylate (Intermediate AD)

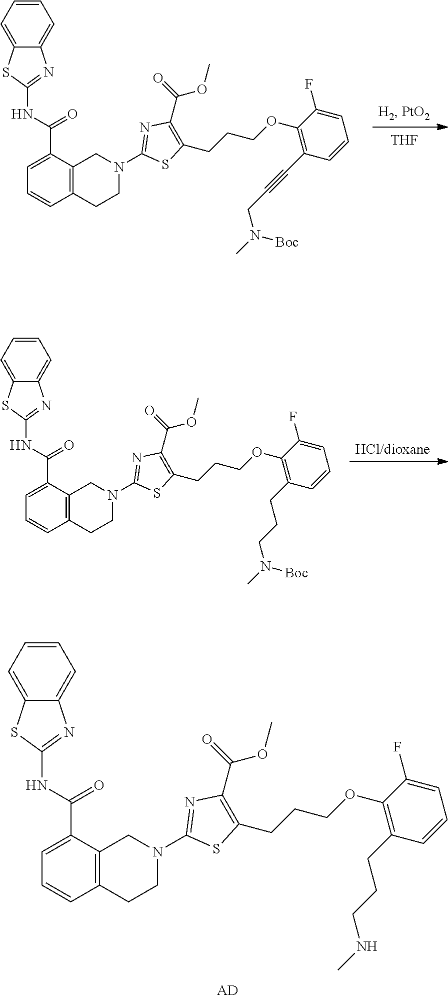

### Step 1—Methyl 2-(8-(benzo[d]thiazol-2-ylcarbamoyl)-3,4-dihydroisoquinolin-2 (1H)-yl)-5-(3-(2-(3-((tert-butoxycarbonyl)(methyl)amino)propyl)-6-fluorophenoxy)propyl)thiazole-4-carboxylate

To a solution of PtO₂ (294.94 mg, 1.30 mmol) in THF (30 mL) was added methyl 2-[8-(1,3-benzothiazol-2-ylcarbamoyl)-3,4-dihydro-1H-isoquinolin-2-yl]-5-[3-[2-[3-[tert-butoxycarbonyl(methyl)amino]prop-1-ynyl]-6-fluoro-phenoxy]propyl]thiazole-4-carboxylate (1 g, 1 mmol, synthesized via Step 1—2 of Intermediate AB) at 20° C., under nitrogen flow. Then the reaction was stirred at 20° C., for 48 h under hydrogen atmosphere (50 psi). On completion, the reaction was filtered to get the filtrate and concentrated to give a residue. The residue was purified by column chromatography on silica gel (eluted with petroleum ether:THF=100:1 to 100:50) to give the title compound (700 mg, 69.6% yield) as yellow oil. ¹H NMR (400 MHz, CDCl₃) δ=11.53-10.78 (m, 1H), 7.77 (br dd, J=3.8, 5.1 Hz, 1H), 7.48 (br d, J=7.5 Hz, 1H), 7.35-7.20 (m, 4H), 7.17-7.08 (m, 1H), 6.82 (br d, J=4.9 Hz, 3H), 4.83 (s, 2H), 4.02-3.95 (m, 2H), 3.80 (br t, J=5.8 Hz, 2H), 3.73 (s, 3H), 3.28-3.07 (m, 4H), 2.96 (brt, J=5.6 Hz, 2H), 2.75 (br s, 3H), 2.60-2.47 (m, 2H), 2.11-1.99 (m, 2H), 1.79-1.68 (m, 2H), 1.34 (s, 9H).

### Step 2—Methyl 2-(8-(benzo[d]thiazol-2-ylcarbamoyl)-3,4-dihydroisoquinolin-2 (1H)-yl)-5-(3-(2-fluoro-6-(3-(methylamino)propyl)phenoxy)propyl)thiazole-4-carboxylate

To a solution of methyl 2-[8-(1,3-benzothiazol-2-ylcarbamoyl)-3,4-dihydro-IH-isoquinolin-2-yl]-5-[3-[2-[3-[tert-butoxycarbonyl(methyl)amino]propyl]-6-fluoro-phenoxy]propyl]thiazole-4-carboxylate (690 mg, 891 umol) in HCl/dioxane (4 M, 10 mL) at 20° C., under nitrogen flow. Then the reaction was stirred at 20° C., for 10 min under nitrogen atmosphere. On completion, the reaction was concentrated to give the title compound (1 g) as yellow solid. LC-MS (ESI+) m/z 674.5 (M+H)⁺.

### 6-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]hexanoic acid (Intermediate AE)

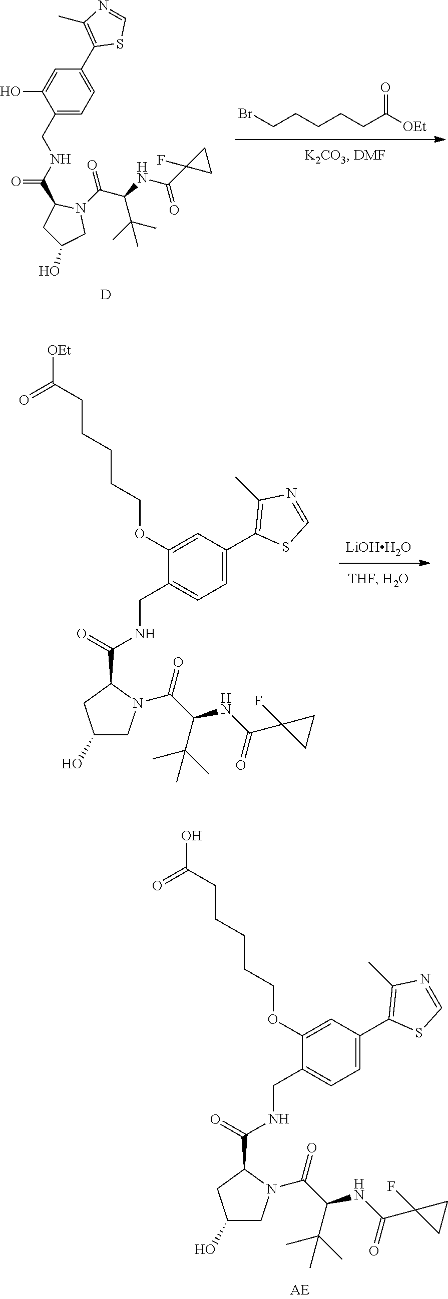

### Step 1—Ethyl 6-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)hexanoate

To a solution of ethyl 6-bromohexanoate (942 mg, 4.22 mmol, 748 uL, CAS #25542-62-5) and (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (1.5 g, 2.82 mmol, Intermediate D) in DMF (20 mL) was added K₂CO₃ (1.17 g, 8.45 mmol). The mixture was then stirred at 70° C., for 12 h. On completion, the reaction mixture was poured into ice water (20 mL) and extracted with EtOAc (40 mL×3). The combined organic layers were washed with brine (40 mL×3), dried over sodium sulfate, filtered and concentrated under reduced pressure to give the title compound (1.5 g) as a yellow oil. LC-MS (ESI⁺) m z 675.3 (M+H)⁺.

### Step 2—6-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)hexanoic acid

To a solution of ethyl 6-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)hexanoate (1 g, 1.48 mmol) in THF (10 mL) and H₂O (2.5 mL) was added LiOH·H₂O (249 mg, 5.93 mmol) at 0° C. The mixture was then stirred at 25° C., for 12 h. On completion, the reaction mixture was poured into water (20 mL) and extracted with DCM (50 mL×3) and the aqueous phase was acidified with 1N HCl to pH=4. Then the mixture was extracted with DCM (50 mL×3), the combined organic layers were washed with brine (50 mL×3), dried over Na₂SO₄, filtered and concentrated under reduced pressure to give the title compound (600 mg) as a yellow solid. ¹H NMR (400 MHZ, DMSO-d₆) δ 12.08-11.90 (m, 1H), 8.98 (s, 1H), 8.49 (br t, J=6.0 Hz, 1H), 7.40 (d, J=7.6 Hz, 1H), 7.29 (br dd, J=2.4, 9.6 Hz, 1H), 7.02-6.98 (m, 1H), 6.94 (br d, J=7.8 Hz, 1H), 4.60 (br d, J=8.8 Hz, 1H), 4.51 (br t, J=8.4 Hz, 1H), 4.35 (br s, 1H), 4.33-4.25 (m, 1H), 4.24-4.15 (m, 1H), 4.04 (br t, J=6.0 Hz, 2H), 3.70-3.57 (m, 2H), 2.45 (s, 4H), 2.27-2.20 (m, 3H), 2.09 (br dd, J=8.0, 11.8 Hz, 1H), 1.96-1.88 (m, 1H), 1.77 (dt, J=6.8, 13.8 Hz, 3H), 1.62-1.53 (m, 3H), 1.50-1.43 (m, 2H), 1.41-1.32 (m, 2H), 1.26-1.20 (m, 2H), 0.96 (s, 9H).

### (S)-1-(4-ethynylphenyl)ethan-1-amine Intermediate AF

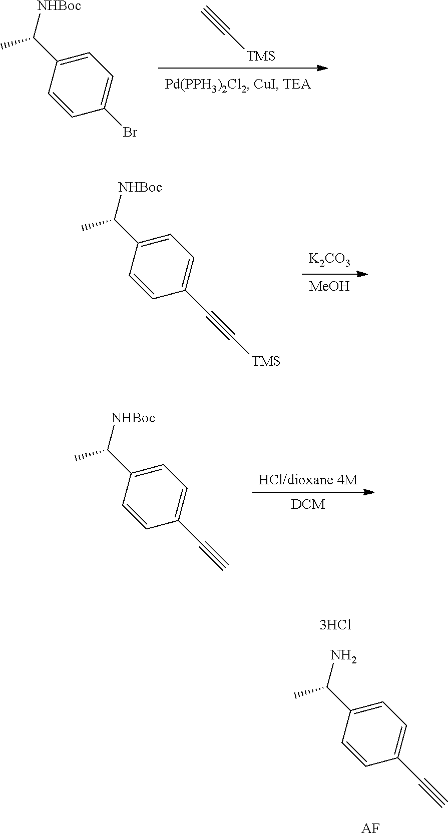

### Step 1—Tert-butyl(S)-(1-(4-((trimethylsilyl)ethynyl)phenyl)ethyl)carbamate

To a solution of tert-butyl(S)-(1-(4-bromophenyl)ethyl)carbamate (150 g, 499 mmol, CAS #847728-89-6) and ethynyltrimethylsilane (147 g, 1.50 mol) in TEA (1.50 L) was added CuI (1.95 g, 10.2 mmol) and Pd(PPh₃)₂Cl₂ (17.4 g, 24.9 mmol) under N₂, then the mixture was stirred at 80° C., for 16 hrs. On completion, the mixture was cooled to 20° C., filtered and EtOAc (2.50 L) was added, and the mixture was washed with water (3.00 L×4). The organic layer was washed with brine (1.00 L), dried over Na₂SO₄, filtered and concentrated to give a residue. The residue was purified by silica gel chromatography eluted with Petroleum ether/Ethyl acetate to give the title compound (114 g, 72% yield) as a yellow oil. LC-MS (ESI+) m/z 262.2 (M-55)⁺. ¹HNMR 400 MHZ, CDCl₃ δ 7.39-7.47 (m, 2H), 7.23 (d, J=8.16 Hz, 2H), 4.79 (br s, 2H), 1.41 (br s, 12H), 0.25 (s, 9H).

### Step 2—Tert-butyl(S)-(1-(4-ethynylphenyl)ethyl)carbamate

A solution of tert-butyl(S)-(1-(4-((trimethylsilyl)ethynyl)phenyl)ethyl)carbamate (95.0 g, 299 mmol) and K₂CO₃ (86.1 g, 623 mmol) in MeOH (700 mL) was stirred at 25° C., for 2 hrs. On completion, the mixture was filtered and concentrated to give the title compound (75.0 g) as a gray solid. LC-MS (ESI+) m z 190.1 (M-55)⁺. ¹HNMR 400 MHZ, CDCl₃ δ 7.44 (d, J=8.07 Hz, 2H), 7.25 (br s, 2H), 4.73 (br d, J=17.61 Hz, 1H), 3.24-3.31 (m, 1H), 1.40 (br s, 12H).

### Step 3—(S)-1-(4-ethynylphenyl)ethan-1-amine

To a solution of HCl/dioxane (4 M, 300 mL) was added a solution of Tert-butyl(S)-(1-(4-ethynylphenyl)ethyl)carbamate (74.0 g, 301 mmol) in DCM (700 mL), and the mixture was stirred at 25° C. for 1 hr. On completion, the mixture was concentrated to give the title compound (80.0 g, 3 HCl) as a gray solid. ¹HNMR (400 MHZ, CDCl₃) δ 8.72 (br s, 3H), 7.54 (q, J=7.82 Hz, 4H), 4.40 (br s, 1H), 4.24 (s, 1H), 1.51 (br d, J=6.85 Hz, 3H).

### Methyl 2-(8-(benzo[d]thiazol-2-ylcarbamoyl)-3,4-dihydroisoquinolin-2 (1H)-yl)-5-(3-(2-fluoro-6-(3-(methylamino)prop-1-yn-1-yl)phenoxy)propyl)thiazole-4-carboxylate (Intermediate AG)

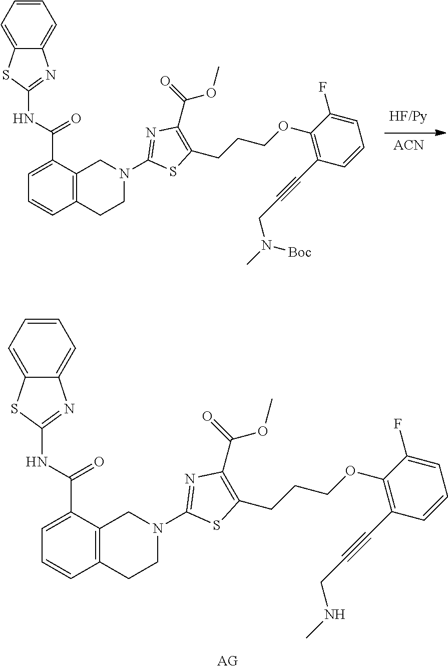

To a solution of methyl 2-[8-(1,3-benzothiazol-2-ylcarbamoyl)-3,4-dihydro-1H-isoquinolin-2-yl]-5-[3-[2-[3-[tert-butoxycarbonyl(methyl)amino]prop-1-ynyl]-6-fluoro-phenoxy]propyl]thiazole-4-carboxylate (400 mg, 519 umol, synthesized via Step 1—2 of Intermediate AB) in ACN (8 mL) was added pyridine; hydrofluoride (3.09 g, 31.1 mmol) at 20° C., under nitrogen flow. Then the reaction was stirred at 20° C., for 1 min under nitrogen atmosphere. On completion, the reaction was under lyophilization to obtain the title compound (800 mg) as a brown solid. LC-MS (ESI+) m/z 670.1 (M+H)⁺.

### Methyl 2-chloro-5-(3-hydroxypropyl)thiazole-4-carboxylate (Intermediate AH)

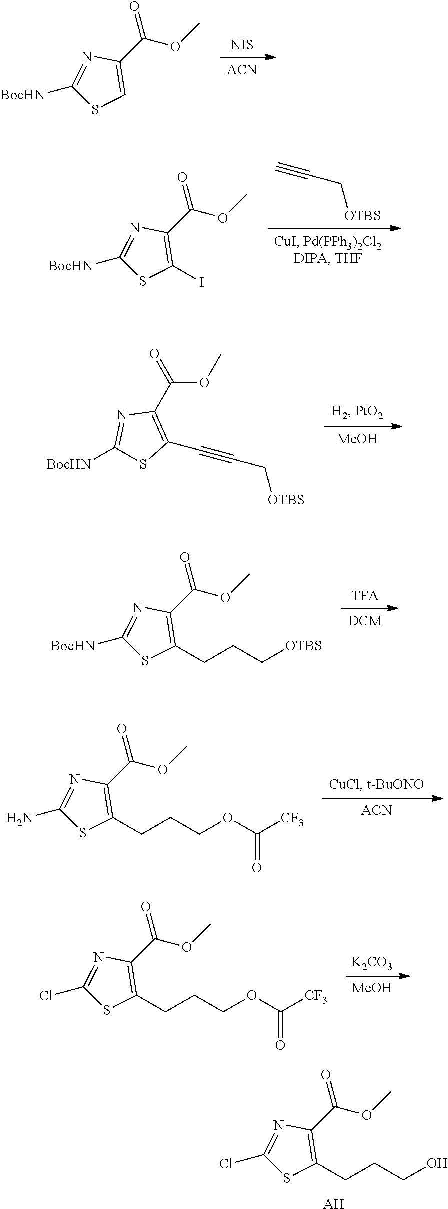

### Step 1—Methyl 2-((tert-butoxycarbonyl)amino)-5-iodothiazole-4-carboxylate

A solution of methyl 2-(tert-butoxycarbonylamino) thiazole-4-carboxylate (20 g, 77.4 mmol, CAS #850429-62-8) in ACN (240 mL) was added NIS (61.0 g, 271 mmol), then the mixture was stirred at 25° C., for 12 h. On completion, the reaction mixture was diluted with saturated brine (100 ml), then it was extracted with EtOAc (50 ml×3). The combined organic layers were extracted with 1 M Na₂S203 (100 ml×3), then with brine again, dried over Na₂SO₄, filtered and the filtrate was concentrated under reduced pressure. The residue was purified by column chromatography (SiO₂, Petroleum ether/Ethyl acetate=10:1-1:1) to give the title compound (36 g) as a white solid. ¹H NMR (400 MHZ, DMSO-d₆) δ=12.01 (br s, 1H), 3.78 (s, 3H), 1.47 (s, 9H).

### Step 2—Methyl 2-((tert-butoxycarbonyl)amino)-5-(3-((tert-butyldimethylsilyl)oxy)prop-1-yn-1-yl) thiazole-4-carboxylate

To a solution of methyl 2-(tert-butoxycarbonylamino)-5-iodo-thiazole-4-carboxylate (100 g, 208 mmol) and tert-butyl-dimethyl-prop-2-ynoxy-silane (70.9 g, 416.46 mmol) in THF (1000 mL) was added DIPA (105.35 g, 1.04 mol), Pd(PPh₃)₂Cl₂ (7.31 g, 10.41 mmol) and CuI (1.98 g, 10.4 mmol) under N₂. The mixture was stirred at 60° C., for 12 h. On completion, silica gel was added to the reaction mixture and the volatiles were removed under reduced pressure. The residue was purified by column chromatography (SiO₂, Petroleum ether/Ethyl acetate=20:1-5:1) to give the title compound (87 g, 98% yield) as a yellow solid. ¹H NMR (400 MHZ, DMSO-d₆) δ=12.19-12.00 (m, 1H), 4.61 (s, 2H), 3.79 (s, 3H), 1.48 (s, 9H), 0.89 (s, 9H), 0.13 (s, 6H).

### Step 3—Methyl 2-((tert-butoxycarbonyl)amino)-5-(3-((tert-butyldimethylsilyl)oxy)propyl)thiazole-4-carboxylate

The solution of methyl 2-(tert-butoxycarbonylamino)-5-]3-[tert-butyl(dimethyl)silyl]oxyprop-1-ynyl]thiazole-4-carboxylate (30 g, 70.3 mmol) in MeOH (300 mL) was added PtO₂ (3.19 g, 14.06 mmol) under H₂ (1.5 g, 743 mmol), and the mixture was stirred at 25° C., for 48 h. On completion, the mixture was filtered, the filter liquor was concentrated under reduced pressure to give a residue to give the title compound (27 g, 89% yield) as a black solid. ¹H NMR (400 MHZ, DMSO-d₆) δ=11.81-11.48 (m, 1H), 3.76 (s, 3H), 3.62 (t, J=6.4 Hz, 2H), 3.14-3.07 (m, 2H), 1.82-1.72 (m, 2H), 1.46 (s, 9H), 0.87-0.85 (m, 9H), 0.04-0.01 (m, 6H).

### Step 4—Methyl 2-amino-5-(3-(2,2,2-trifluoroacetoxy)propyl)thiazole-4-carboxy late

To a solution of methyl 2-(tert-butoxycarbonylamino)-5-[3-[tert-butyl (dimethyl)silyl]oxypropyl]thiazole-4-carboxylate (27 g, 62.7 mmol) in DCM (135 mL) was added TFA (54 mL), then the mixture was stirred at 25° C., for 12 h. On completion, the mixture was concentrated under reduced pressure to give the title compound (34 g) as a black oil. LC-MS (ESI) m/z 312.9 (M+H).

### Step 5—Methyl 2-chloro-5-(3-(2,2,2-trifluoroacetoxy)propyl)thiazole-4-carboxylate

To a solution of methyl 2-amino-5-[3-(2,2,2-trifluoroacetyl)oxypropyl]thiazole-4-carboxylate (13 g, 41.6 mmol) and CuCl (6.18 g, 62.5 mmol) in ACN (130 mL) was added to tert-butyl nitrite (8.59 g, 83.3 mmol). The mixture was stirred at 25° C., for 2 h. On completion, the reaction mixture was poured into water (50 mL), and then extracted with EtOAc (30 mL×3). The combined organic layers were washed with brine (30 mL×3), dried over Na₂SO₄, filtered and concentrated under reduced pressure to give a residue. The residue was purified by column chromatography (SiO₂, Petroleum ether/Ethyl acetate=10/1-1/1) to give the title compound (7.4 g, 54% yield) as a black oil. LC-MS (ESI) m/z 332.0 (M+H).

### Step 6-Methyl 2-chloro-5-(3-hydroxypropyl)thiazole-4-carboxylate

To a solution of methyl 2-chloro-5-(3-(2,2,2-trifluoroacetoxy)propyl)thiazole-4-carboxylate (7.40 g, 22.3 mmol) and K₂CO; (6.17 g, 44.6 mmol) in MeOH (74 mL), then the mixture was stirred at 20° C., for 1 h. On completion, the reaction mixture was quenched with H₂O (100 mL) and then extracted with EtOAc (100 mL×3). The combined organic layers were washed with brine (100 mL×3), dried over Na₂SO₄, filtered and concentrated under reduced pressure to give the title compound (5.00 g, crude) as a yellow oil. ¹H NMR (400 MHZ, DMSO-d₆) δ=4.59 (t, J=5.2 Hz, 1H), 3.83-3.80 (m, 3H), 3.44 (q, J=6.0 Hz, 2H), 3.21-3.15 (m, 2H), 1.79-1.71 (m, 2H).

### Methyl 2-chloro-5-(3-(2-fluoro-4-iodophenoxy)propyl)thiazole-4-carboxylate (Intermediate AI)

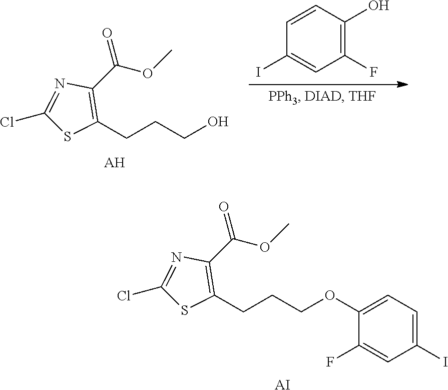

To a solution of methyl methyl 2-chloro-5-(3-hydroxypropyl)thiazole-4-carboxylate (1.30 g, 5.52 mmol), Intermediate AH, 2-fluoro-4-iodo-phenol (1.97 g, 8.27 mmol) and PPh; (2.17 g, 8.27 mmol) in THF (29.3 mL) was added DIAD (1.67 g, 8.27 mmol) and THF (13 mL) at 0° C., under N₂. Then the reaction was stirred at 20° C., for 10 h under N₂ atmosphere. On completion, the reaction mixture was quenched with NaHCO; (40 mL) and then extracted with EtOAc (40 mL×3). The combined organic layers were washed with brine (40 mL×3), dried over Na₂SO₄, filtered and concentrated under reduced pressure to give a residue. The residue was purified by flash silica gel chromatography (Ethyl acetate/Petroleum ether=1/5 to 1/4) to give the title compound (2.10 g, 82% yield) as a white solid. LC-MS (ESI+) m/z 455.8 (M+H)⁺.

### Methyl 2-(8-(benzo[d]thiazol-2-ylcarbamoyl)-3,4-dihydroisoquinolin-2 (1H)-yl)-5-(3-(2-fluoro-4-iodophenoxy)propyl)thiazole-4-carboxylate (Intermediate AJ)

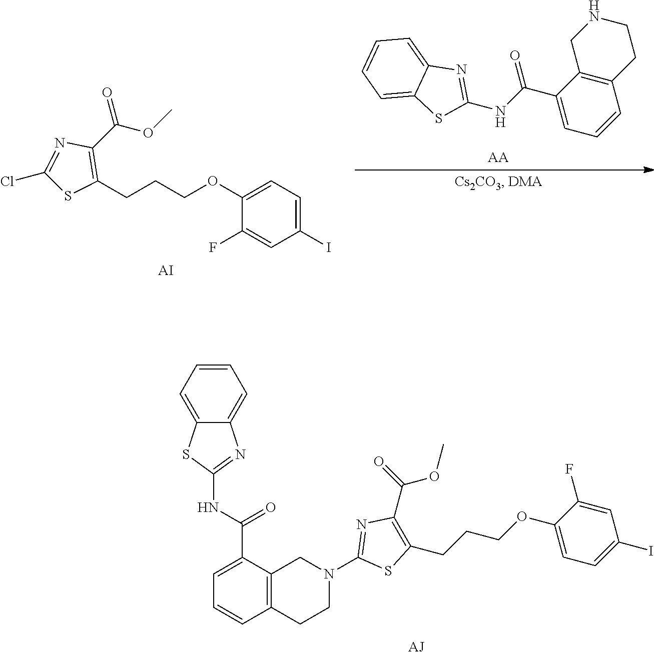

### Step 1—Methyl 2-(8-(benzo[d]thiazol-2-ylcarbamoyl)-3,4-dihydroisoquinolin-2 (1H)-yl)-5-(3-(2-fluoro-4-iodophenoxy)propyl)thiazole-4-carboxylate

A solution of N-(benzo[d]thiazol-2-yl)-1,2,3,4-tetrahydroisoquinoline-8-carboxamide (1.40 g, 4.52 mmol, Intermediate AA), methyl 2-chloro-5-(3-(2-fluoro-4-iodophenoxy)propyl)thiazole-4-carboxylate (2.10 g, 4.52 mmol, Intermediate AI) and Cs₂CO₃ (2.94 g, 9.03 mmol) in DMA (22 mL) was degassed and purged with N₂ three times. Then the mixture was stirred at 100° C., for 12 h under N₂ atmosphere. On completion, the reaction mixture was filtered and concentrated under reduced pressure to give a residue. This product was purified by flash silica gel chromatography (Ethyl acetate/THF=1/1) to give the title compound (2.08 g, 57% yield) as a red solid. LC-MS (ESI+) m/z 729.3 (M+H)⁺.

### 2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carboxylic acid (Intermediate AK)

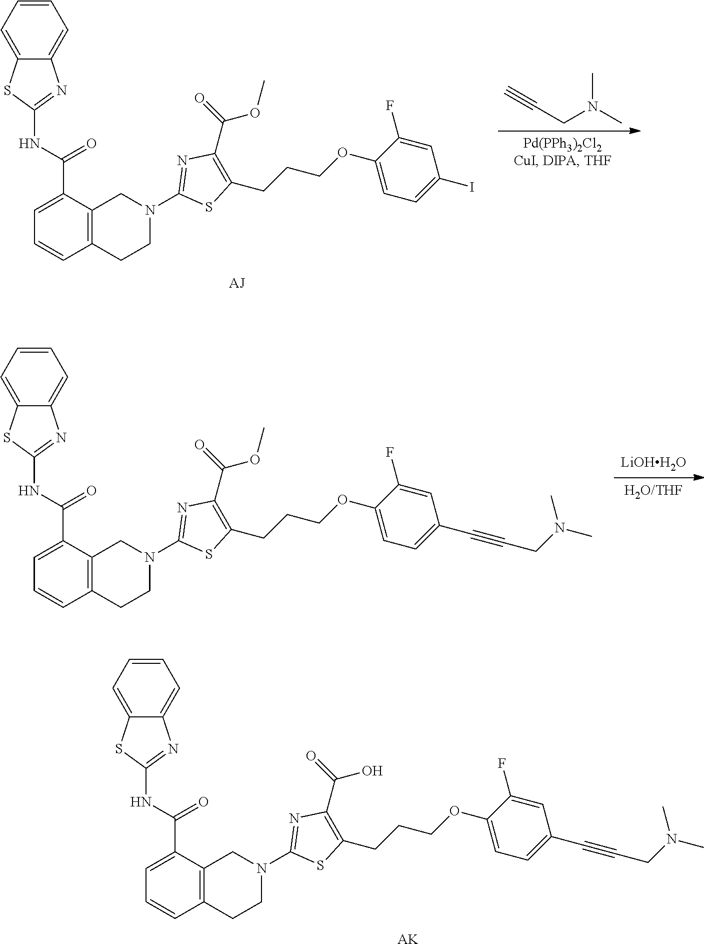

### Step 1—Methyl 2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carboxylate

A solution of methyl 2-(8-(benzo[d]thiazol-2-ylcarbamoyl)-3,4-dihydroisoquinolin-2 (1H)-yl)-5-(3-(2-fluoro-4-iodophenoxy)propyl)thiazole-4-carboxylate (1.40 g, 1.73 mmol, Intermediate AJ), N,N-dimethylprop-2-yn-1-amine (288 mg, 3.46 mmol), Pd(PPh₃)₂Cl₂ (121 mg, 173 umol), CuI (32.9 mg, 173 umol) and DIPA (1.22 g, 12.1 mmol) in THF (28 mL) was degassed and purged with N₂ three times. Then the mixture was stirred at 60° C., for 3 h under N₂ atmosphere. On completion, the reaction mixture was quenched with H₂O (30 mL), and then extracted with EtOAc (30 mL×3). The combined organic layers were washed with brine (30 mL×3), dried over Na₂SO₄, filtered and concentrated under reduced pressure to give a residue. This product was purified by flash silica gel chromatography (Petroleum ether/Ethyl acetate=0/1) to give the title compound (970 mg, 74% yield) as a red solid. LC-MS (ESI+) m/z 684.4 (M+H)+.

### Step 2—2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carboxylic acid

A solution of methyl 2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carboxylate (870 mg, 1.27 mmol) and LiOH·H₂O (107 mg, 2.54 mmol) in THF (7.2 mL) and H₂O (1.8 mL) was stirred at 20° C., for 12 h. On completion, the reaction mixture was quenched with HCl (2M) the pH was adjusted to 3˜4, then the mixture was addition H₂O (10 mL) and filtered and concentrated under reduced pressure to give a residue. The residue was purified by re-crystallization from ethyl acetate (20 mL) at 20° C., to give the title compound (750 mg, 70% yield) as a white solid. LC-MS (ESI+) m/z 670.3 (M+H)⁺.

### Ethyl 6-sulfamoylhexanoate (Intermediate AL)

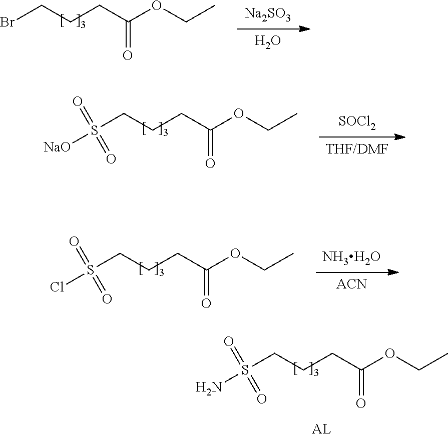

### Step 1—Sodium 6-ethoxy-6-oxohexane-1-sulfonate

A solution of ethyl 6-bromohexanoate (20.0 g, 89.6 mmol) and Na₂SO; (14.7 g, 117 mmol) in H₂O (100 mL) was stirred at 120° C., for 12 h. On completion, the reaction mixture was lyophilized to afford the title compound (21.0 g) as a white solid. ¹H NMR (400 MHZ, DMSO-d₆) δ=4.02 (q, J=7.2 Hz, 2H), 2.46-2.41 (m, 2H), 2.24 (t, J=7.2 Hz, 2H), 1.60-1.52 (m, 2H), 1.51-1.45 (m, 2H), 1.33-1.25 (m, 2H), 1.15 (t, J=7.2 Hz, 3H).

### Step 2—Ethyl 6-(chlorosulfonyl) hexanoate

A solution of sodium 6-ethoxy-6-oxohexane-1-sulfonate (21.0 g, 91.2 mmol) in THF (210 mL) and DMF (10.5 mL) was added SOCl₂ (93.9 g, 789 mmol) dropwise at 0° C., under N₂ and the mixture was heated to 70° C., and stirred for 1 h. On completion, the mixture was concentrated in vacuo to afford the title compound (22.14 g) in DMF (10.5 mL) as a yellow liquid.

### Step 3—Ethyl 6-sulfamoylhexanoate

Ethyl 6-(chlorosulfonyl) hexanoate (22.14 g, 91.2 mmol) in DMF (10.5 mL) was diluted with ACN (200 mL). The resulting suspension was added into NH₃·H₂O (137 g, 974 mmol) at 0° C., under N₂ and stirred for 30 min. On completion, the reaction mixture was diluted with EtOAc (200 mL). The combined organic layers were washed with H₂O (50 mL×2), brine (50 mL×2), dried over Na₂SO₄ and evaporated. The residue was purified by column chromatography (SiO₂, PE/EA=3/1) to afford the title compound (14.2 g, 56% yield) as a yellow oil. ¹H NMR (400 MHZ, CDCl₃) δ=4.89 (s, 2H), 4.15-4.10 (m, 2H), 3.16-3.11 (m, 2H), 2.36-2.31 (m, 2H), 1.91-1.84 (m, 3H), 1.71-1.64 (m, 3H), 1.53-1.45 (m, 3H), 1.26 (t, J=7.2 Hz, 3H).

### 6-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl)hexanoic acid (Intermediate AM)

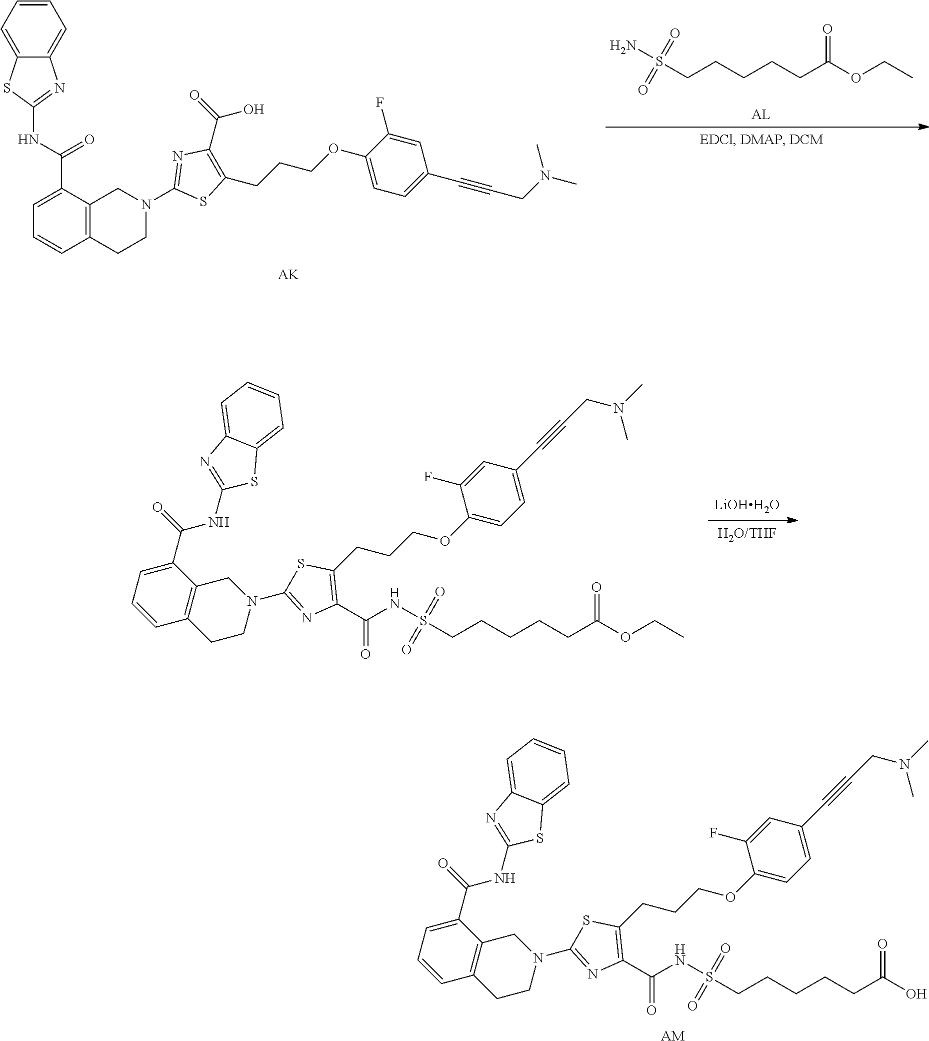

### Step 1—Ethyl 6-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl)hexanoate

To a solution of 2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carboxylic acid (50.0 mg, 74.7 umol, Intermediate AK), ethyl 6-sulfamoylhexanoate (41.7 mg, 149 umol, Intermediate AL), EDCI (28.6 mg, 0.149 mmol) and DMAP (18.2 mg, 0.149 mmol) in DCM (4 mL) was degassed and purged with N₂ three times and then the mixture was stirred at 25° C., for 12 h under N₂ atmosphere. On completion, the reaction mixture was filtered and concentrated under reduced pressure to give a residue. The residue was purified by prep-TLC (SiO₂, DCM:MeOH=10:1) to give the title compound (140 mg) as a green oil. LC-MS (ESI+) m/z 875.5 (M+H)⁺.

### Step 2—6-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl)hexanoic acid

To a solution of ethyl 6-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl)hexanoate (140 mg, 160 umol) and LiOH·H₂O (26.8 mg, 640 umol) in H₂O (0.2 mL) and THF (0.8 mL) was stirred at 20° C., for 2 h. On completion, the reaction mixture was quenched with H₂O (1 mL) and then extracted with EtOAc (1 mL×3). The H₂O layer was adjusted to pH 3-4 with HCl (2M), then the mixture was addition EtOAc (2 mL) and filtered and concentrated under reduced pressure to give the title compound (90.0 mg) as a white solid. LC-MS (ESI+) m/z 847.2 (M+H)⁺. Ethyl 9-sulfamoylnonanoate (Intermediate AN)

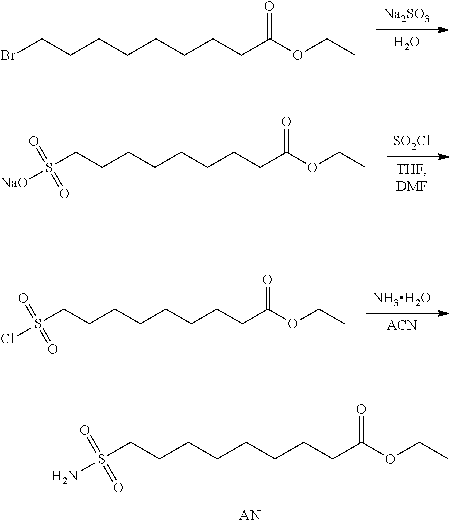

### Step 1—(9-ethoxy-9-oxo-nonyl)sulfonyloxysodium

A solution of ethyl 9-bromononanoate (2.00 g, 7.54 mmol, CAS #28598-81-4) and Na₂SO₃ (1.24 g, 9.80 mmol) in H₂O (10 mL) was stirred at 120° C., for 12 h. On completion, the mixture was cooled and lyophilization to give the title compound (2.17 g) as a white solid. ¹H NMR (400 MHZ, DMSO-d₆) δ4.04 (q, J=7.2 Hz, 2H), 2.41-2.34 (m, 2H), 2.26 (t, J=7.6 Hz, 2H), 1.59-1.47 (m, 4H), 1.24 (br s, 8H), 1.17 (t, J=7.2 Hz, 3H).

### Step 2—Ethyl 9-(chlorosulfonyl) nonanoate

To a solution (9-ethoxy-9-oxo-nonyl)sulfonyloxysodium (2.17 g, 7.53 mmol) in DMF (1 mL) and THF (20 mL). The mixture was cooled to 0° C., under N₂ and SOCl₂ (7.74 g, 4.72 mL) was added dropwise. Then it was heated to 70° C., and stirred for 1 h. On completion, the solution was concentrated in vacuo to give the title compound (2.14 g) as a white solid.

### Step 3—Ethyl 9-sulfamoylnonanoate

A suspension of ethyl 9-chlorosulfonylnonanoate (2.14 g, 7.51 mmol) in ACN (20 mL) was added into ammonium: hydroxide (11.4 g, 12.6 mL) at 0° C., and stirred for 1 h. On completion, the reaction mixture was quenched with NH₄Cl (10 mL) and extracted with EtOAC (50 mL×2). The combined organic layers were dried over Na₂SO₄ and evaporated. The residue was purified by silica gel column chromatography (Petroleum ether:Ethyl acetate=7:1˜2:1) to give the title compound (1.20 g, 60% yield) as a white solid. ¹H NMR (400 MHZ, CDCl₃) δ 4.86 (s, 2H), 4.12 (q, J=7.2 Hz, 2H), 3.15-3.04 (m, 2H), 2.80 (s, 3H), 2.29 (t, J=7.6 Hz, 2H), 1.88-1.81 (m, 2H), 1.67-1.56 (m, 2H), 1.49-1.38 (m, 2H), 1.32 (br s, 6H), 1.25 (t, J=7.2 Hz, 3H).

### 9-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl) nonanoic acid (Intermediate AO)

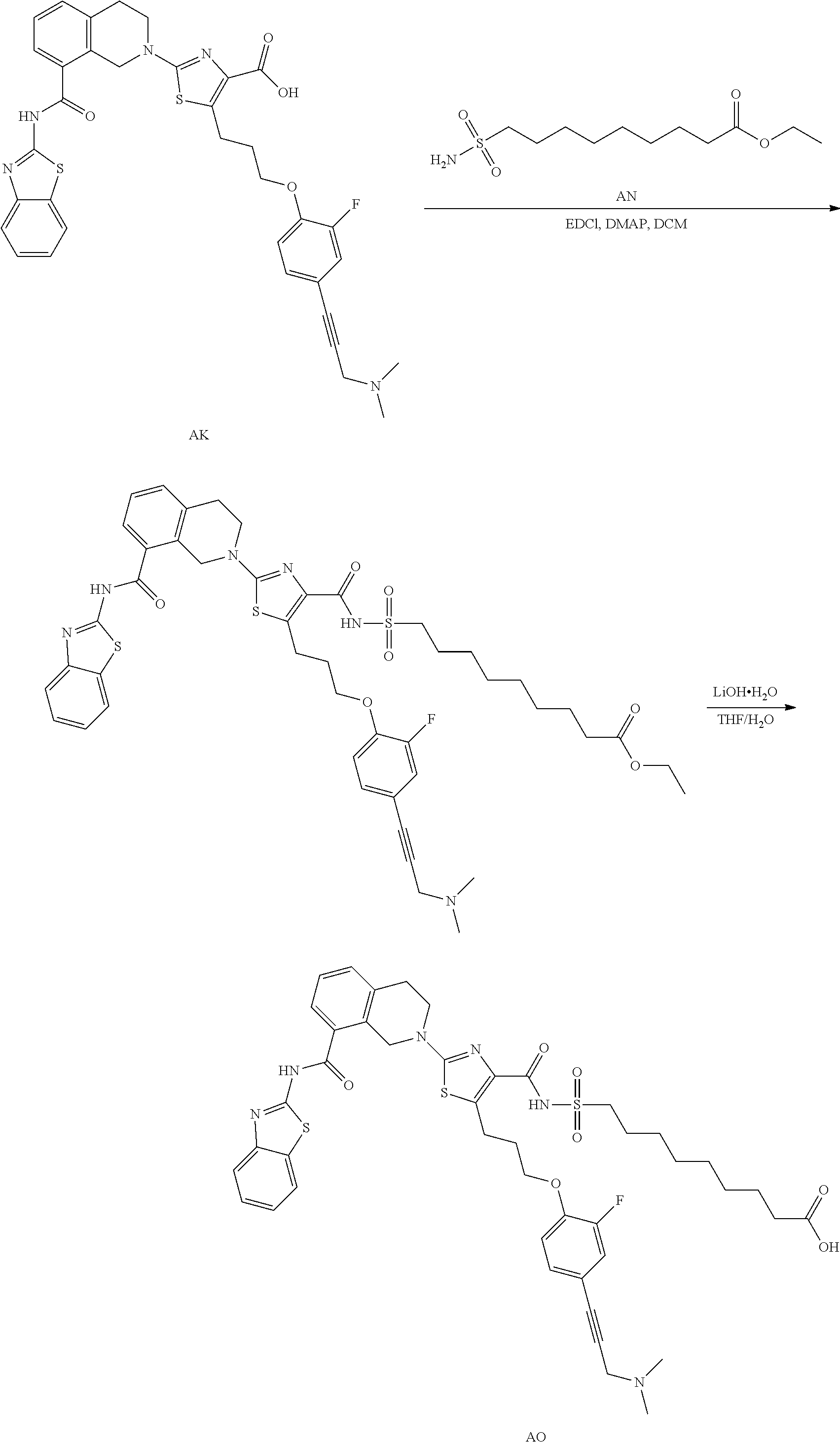

### Step 1—ethyl 9-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl) nonanoate

To a solution of 2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carboxylic acid (100 mg, 149 umol, Intermediate AK), ethyl 9-sulfamoylnonanoate (99.1 mg, 299 umol, Intermediate AN) in DCM (7 mL) was added EDCI (57.2 mg, 299 umol), DMAP (36.5 mg, 299 umol) at 0° C., under N₂, then the mixture was stirred at 25° C., for 17 h. On completion, the reaction mixture was quenched by addition NH₄Cl (2 mL) and extracted with EtOAC (3 mL×3). The combined organic layers were washed with brine (3 mL×3), dried over Na₂SO₄ and evaporated. The residue was purified by prep-HPLC (column: Phenomenex Luna C18 100*30 mm*5 um; mobile phase: [column: YMC Triart C18 250*50 mm*7 um; mobile phase: [water (FA)-ACN]: B %: 5%-70%, 20 min) to give the title compound (63.0 mg, 46% yield) as a white solid. LC-MS (ESI+) m/z 917.3 (M+H)⁺.

### Step 2—9-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl) nonanoic acid

To a solution of ethyl 9-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl) nonanoate (62.9 mg, 68.5 umol) and LiOH·H₂O (23.0 mg, 685 umol) in THF (2 mL), H₂O (0.4 mL), then the mixture was stirred at 25° C., for 18 h. On completion, the reaction mixture was filtered and the pH of the aqueous phase was adjusted to 4˜5 by addition of 2M HCl and extracted with EtOAC (5 mL×2). The combined organic layers were dried over Na₂SO₄ and evaporated to give the title compound (60.0 mg) as a white solid. LC-MS (ESI+) m/z 889.3 (M+H)⁺. 0]3-(3,6-dichloro-5-methylpyridazin-4-yl) propan-1-ol (Intermediate AP)

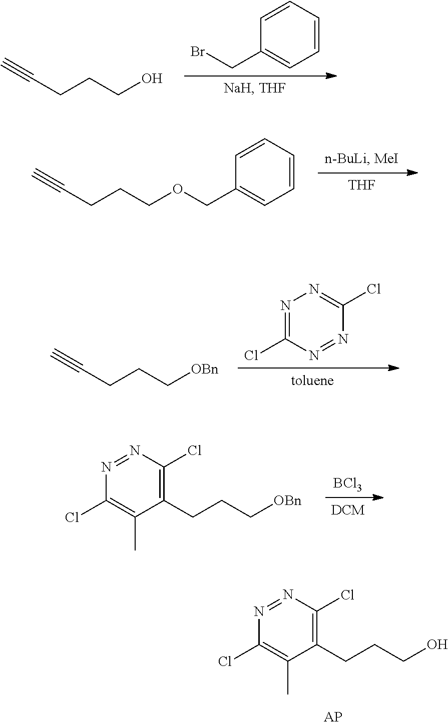

### Step 1—((Pent-4-yn-1-yloxy)methyl)benzene

To a solution of pent-4-yn-1-ol (45 g, 540 mmol) in THF (450 mL) was added NaH (23.5 g, 588 mmol, 60% dispersion in mineral oil) at 0° C., for 1 h, then the (bromomethyl)benzene (84.2 g, 492 mmol, 58.4 mL) was added, then the mixture was stirred at 25° C., for 2 h. On completion, the reaction mixture was quenched with H₂O (500 mL) at 20° C., and extracted with EtOAc (500 mL×3). The combined organic layers were washed with brine (200 mL×2), dried over Na₂SO₄, filtered and concentrated under reduced pressure to give the title compound (100 g) as yellow oil. ¹H NMR (400 MHZ, DMSO-d₆) δ=7.41-7.24 (m, 5H), 4.46 (s, 2H), 3.49 (t, J=6.4 Hz, 2H), 2.74 (t, J=2.8 Hz, 1H), 2.23 (dt, J=2.8, 7.2 Hz, 2H), 1.71 (t, J=6.8 Hz, 2H).

### Step 2—((Hex-4-yn-1-yloxy)methyl)benzene

To a solution of ((pent-4-yn-1-yloxy)methyl)benzene (50 g, 300 mmol) in THF (500 mL) and the reaction was cooled to −78° C., then n-BuLi (2.5 M, 138 mL) was added dropwise over 30 min and the reaction was stirred at −78° C., for 1 h. Then the Mel (61.10 g, 430 mmol, 26.8 mL) was added dropwise and the reaction was warm to 0° C., for 1 h. On completion, the reaction mixture was quenched with H₂O (500 mL) at 20° C., and extracted with EtOAc (500 mL×3). The combined organic layers were washed with brine (500 mL×2), dried over Na₂SO₄, filtered and concentrated under reduced pressure to give the title compound (50 g) as yellow oil. LC-MS (ESI+) m/z 189.3 (M+H)⁺.

### Step 3—4-(3-(Benzyloxy)propyl)-3,6-dichloro-5-methylpyridazine

A solution of ((hex-4-yn-1-yloxy)methyl)benzene (15.0 g, 79.5 mmol) and 3,6-dichloro-1,2,4,5-tetrazine (10 g, 70 mmol) in toluene (30 mL) was stirred at 160° C., for 12 h under N₂ atmosphere. On completion, the reaction mixture was quenched with H₂O (300 mL) at 20° C., and extracted with EtOAc (200 mL×3). The combined organic layers were washed with brine (200 mL×2), dried over Na₂SO₄, filtered and concentrated under reduced pressure to give a residue. The residue was purified by column chromatography (SiO₂, Petroleum ether/Ethyl acetate=10/1 to 3/1) to give the title compound (6 g, 29% yield) as orange gum.

### Step 4—3-(3,6-dichloro-5-methylpyridazin-4-yl) propan-1-ol

To a solution of 4-(3-(benzyloxy)propyl)-3,6-dichloro-5-methylpyridazine (6 g, 19.3 mmol) in DCM (20 mL) was added BCl₃ (1 M, 193 mL) at 0° C., then the mixture was stirred at 25° C., for 4 h. On completion, the reaction mixture was poured into aq, saturated NaHCO₃ (300 mL) to quench, then extracted with DCM (200 mL×3). The combined organic layers were washed with brine (200 mL×2), dried over Na₂SO₄, filtered and concentrated under reduced pressure to give a residue. The residue was purified by column chromatography (SiO₂, Petroleum ether/Ethyl acetate=5/1 to 1/1) to give the title compound (4 g, 93.84% yield) as yellow gum. ¹H NMR (400 MHZ, DMSO-d₆) δ=4.65 (br t, J=4.8 Hz, 1H), 3.48 (q, J=5.6 Hz, 2H), 2.88-2.79 (m, 2H), 2.42 (s, 3H), 1.70-1.59 (m, 2H).

### Methyl 2-(3-chloro-4-methyl-6,7-dihydropyrido[2,3-c]pyridazin-8 (5H)-yl)-5-(3-hydroxypropyl)thiazole-4-carboxylate (Intermediate AQ)

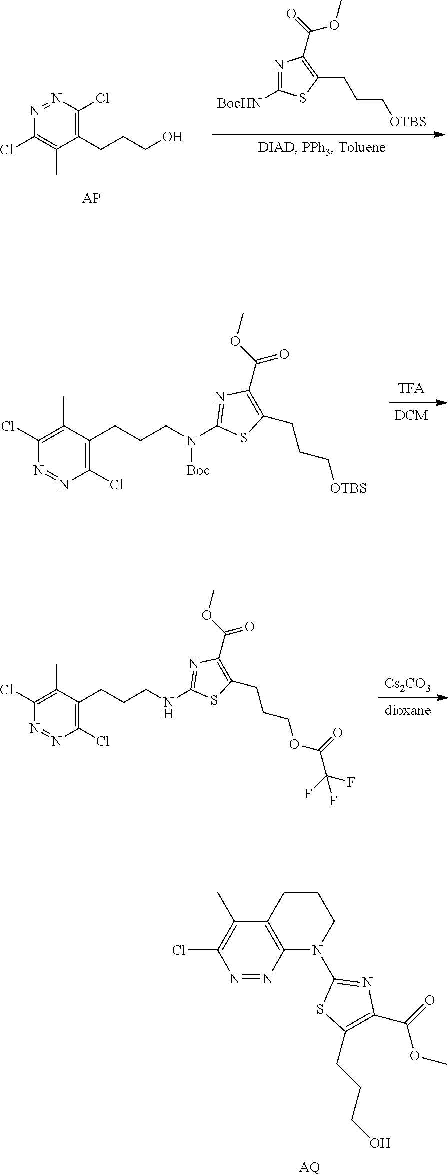

### Step 1 Methyl 2-((tert-butoxycarbonyl)(3-(3,6-dichloro-5-methylpyridazin-4-yl) propyl)amino)-5-(3-((tert-butyldimethylsilyl)oxy)propyl)thiazole-4-carboxylate

To a solution of 3-(3,6-dichloro-5-methylpyridazin-4-yl) propan-1-ol (3.2 g, 15 mmol, Intermediate AP), methyl 2-((tert-butoxycarbonyl)amino)-5-(3-((tert-butyldimethylsilyl)oxy)propyl)thiazole-4-carboxylate (6.23 g, 14.5 mmol, synthesized via Steps 1-3 of Intermediate AH) and PPh; (5.69 g, 21.7 mmol) in toluene (60 mL) was added the solution of DIAD (4.39 g, 21.7 mmol, 4.22 mL) in toluene (10 mL) at 0° C. Then the mixture was stirred at 50° C., for 6 h. On completion, the reaction mixture was quenched with H₂O (60 mL) at 20° C., and extracted with EtOAc (50 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=10/1 to 3/1) to give the title compound (9.1 g, 79% yield) as yellow gum. LC-MS (ESI+) m/z 635.1 (M+H)⁺.

### Step 2—Methyl 2-((3-(3,6-dichloro-5-methylpyridazin-4-yl) propyl)amino)-5-(3-(2,2,2-trifluoroacetoxy)propyl)thiazole-4-carboxylate

To a solution of methyl 2-((tert-butoxycarbonyl)(3-(3,6-dichloro-5-methylpyridazin-4-yl) propyl)amino)-5-(3-((tert-butyldimethylsilyl)oxy)propyl)thiazole-4-carboxylate (9.1 g, 12 mmol) in DCM (90 mL) was added TFA (33.0 g, 289 mmol, 21.41 mL), the mixture was stirred at 25° C. 1 h. On completion, concentrated in vacuo to give the title compound (4.3 g) as brown gum. LC-MS (ESI+) m/z 514.9 (M+H)⁺.

### Step 3—methyl 2-(3-chloro-4-methyl-6,7-dihydropyrido[2,3-c]pyridazin-8 (5H)-yl)-5-(3-hydroxypropyl)thiazole-4-carboxylate

To a solution of methyl methyl 2-((3-(3,6-dichloro-5-methylpyridazin-4-yl) propyl)amino)-5-(3-(2,2,2-trifluoroacetoxy)propyl)thiazole-4-carboxylate (4.3 g, 8.3 mmol) in dioxane (50 mL) was added Cs₂CO; (13.6 g, 41.7 mmol), then the mixture was stirred at 80° C., for 1 h. On completion, the reaction mixture was quenched with H₂O (30 mL) at 20° C., then extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=3/1 to/1) to give the title compound (300 mg, 9% yield) as white solid. LC-MS (ESI+) m z 383.0 (M+H)⁺.

### Methyl 5-(3-(4-(3-((tert-butoxycarbonyl)(methyl)amino)prop-1-yn-1-yl)-2-fluorophenoxy)propyl)-2-(3-chloro-4-methyl-6,7-dihydropyrido[2,3-c]pyridazin-8 (5H)-yl) thiazole-4-carboxylate (Intermediate AR)

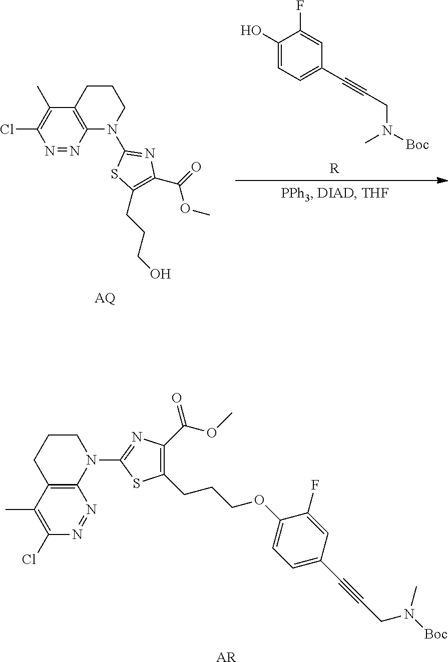

To a solution of methyl 2-(3-chloro-4-methyl-6,7-dihydropyrido[2,3-c]pyridazin-8 (5H)-yl)-5-(3-hydroxypropyl)thiazole-4-carboxylate (120 mg, 310 umol, Intermediate AQ) and tert-butyl (3-(3-fluoro-4-hydroxyphenyl)prop-2-yn-1-yl)(methyl)carbamate (105 mg, 376 umol, Intermediate R), and PPh₃ (123 mg, 470 umol) in THF (5 mL) was dropwise added DIAD (95.1 mg, 470 umol, 91.4 uL) at 0° C. Then the mixture was stirred at 0-25° C., for 12 h. On completion, the reaction mixture was concentrated in vacuo. The residue was purified by flash silica gel chromatography (ISCOR: 12 g SepaFlash® Silica Flash Column, Eluent of 0˜60% Ethyl acetate/Petroleum ethergradient (@ 80 mL/min) to give the title compound (180 mg, 89% yield) as yellow gum. LC-MS (ESI+) m/z 644.3 (M+H)⁺.

### Methyl 2-(3-(benzo[d]thiazol-2-ylamino)-4-methyl-6,7-dihydropyrido[2,3-c]pyridazin-8 (5H)-yl)-5-(3-(2-fluoro-4-(3-(methylamino)prop-1-yn-1-yl)phenoxy)propyl)thiazole-4-carboxylate (Intermediate AS)

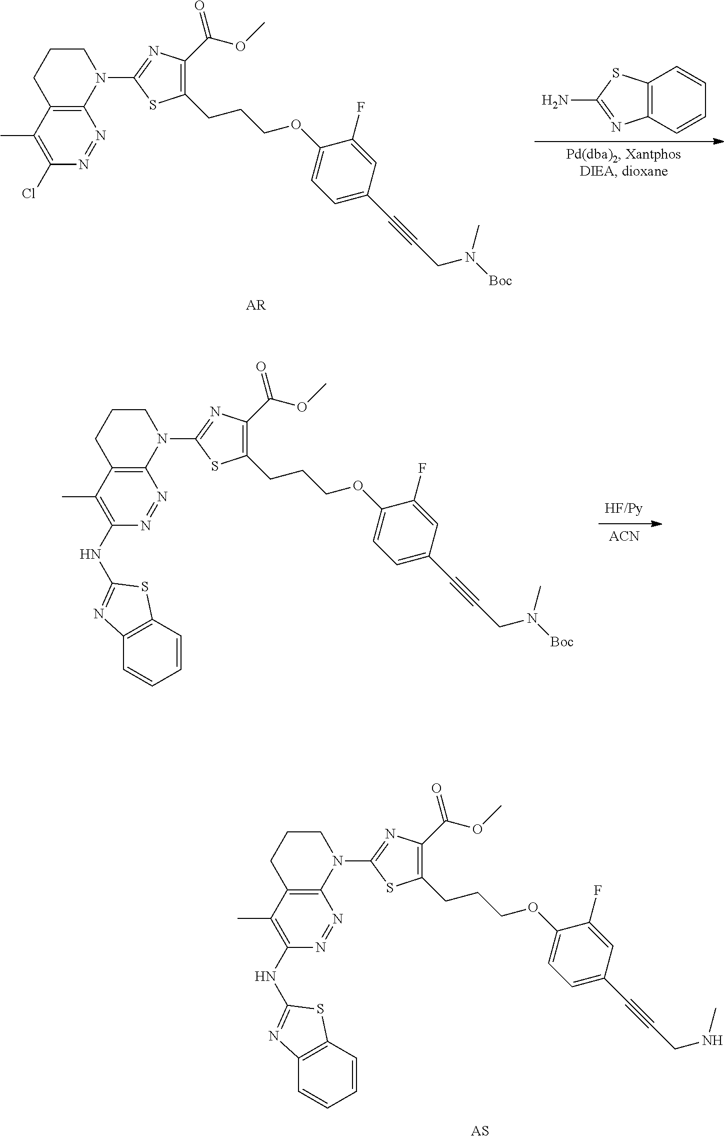

### Step 1—Methyl 2-(3-(benzo[d]thiazol-2-ylamino)-4-methyl-6,7-dihydropyrido[2,3-c]pyridazin-8 (5H)-yl)-5-(3-(4-(3-((tert-butoxycarbonyl)(methyl)amino)prop-1-yn-1-yl)-2-fluorophenoxy)propyl)thiazole-4-carboxylate

To a solution of methyl 5-(3-(4-(3-((tert-butoxycarbonyl)(methyl)amino)prop-1-yn-1-yl)-2-fluorophenoxy)propyl)-2-(3-chloro-4-methyl-6,7-dihydropyrido[2,3-c]pyridazin-8 (5H)-yl) thiazole-4-carboxylate (100 mg, 200 umol, Intermediate AR), benzo[d]thiazol-2-amine (46.6 mg, 310 umol), Xantphos (18.0 mg, 31.1 umol) and DIEA (100 mg, 776 umol, 135.20 uL) in dioxane (2 mL) was added Pd₂(dba)₃ (14.2 mg, 15.5 umol) at N₂ atmosphere. Then the reaction mixture was stirred at 120° C., for 2 h under N₂ atmosphere. On completion, the reaction mixture was quenched with H₂O (2 mL) at 20° C., and extracted with EtOAc (2 mL×3). The combined organic layers were washed with brine (2 mL×2), dried over Na₂SO₄, filtered and concentrated under reduced pressure to give a residue. The residue was purified by flash silica gel chromatography (ISCOR; 4 g SepaFlash® Silica Flash Column, Eluent of 0˜100% Ethyl acetate/Petroleum ethergradient @ 40 mL/min) to give the title compound (100 mg, 85% yield) as brown solid. LC-MS (ESI+) m/z 758.2 (M+H)⁺.

### Step 2—Methyl 2-(3-(benzo[d]thiazol-2-ylamino)-4-methyl-6,7-dihydropyrido[2,3-c]pyridazin-8 (5H)-yl)-5-(3-(2-fluoro-4-(3-(methylamino)prop-1-yn-1-yl)phenoxy)propyl)thiazole-4-carboxylate

To a solution of methyl 2-(3-(benzo[d]thiazol-2-ylamino)-4-methyl-6,7-dihydropyrido[2,3-c]pyridazin-8 (5H)-yl)-5-(3-(4-(3-((tert-butoxycarbonyl)(methyl)amino)prop-1-yn-1-yl)-2-fluorophenoxy)propyl)thiazole-4-carboxylate (100 mg, 100 umol) in ACN (1 mL) was added pyridine; hydrofluoride (784 mg, 7.92 mmol, 713 uL), then the mixture was stirred at 25° C., for 2 min. On completion, the reaction mixture was concentrated in vacuo to give the title compound (80 mg) as brown solid. LC-MS (ESI+) m/z 658.4 (M+H)⁺.

### (2S,4R)-1-((S)-2-(1-fluorocyclopropanecarboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide (Intermediate AT)

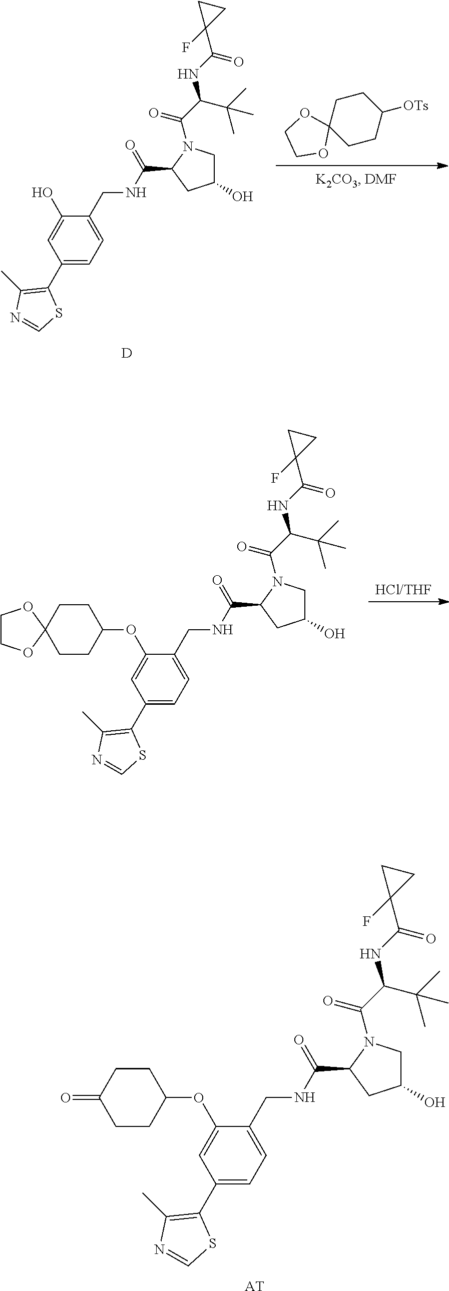

### Step 1—(2S,4R)—N-(2-(1,4-dioxaspiro[4.5]decan-8-yloxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (100 mg, 188 umol, Intermediate D) and 1,4-dioxaspiro[4.5]decan-8-yl 4-methylbenzenesulfonate (117 mg, 376 umol, CAS #23511 May 9) in DMF (2 mL) was added K₂CO; (130 mg, 939 umol), then the mixture was stirred at 90° C. for 12 h. On completion, the reaction mixture was poured into water (1 mL), and then extracted with EtOAc (1 mL×3). The combined organic layers were washed with brine (1 mL×3), dried over Na₂SO₄, filtered and concentrated under reduced pressure to give the title compound (130 mg) as a white solid. LC-MS (ESI⁺) m/z 673.6 (M+H)⁺.

### Step 2—(2S,4R)-1-((S)-2-(1-fluorocyclopropanecarboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide

To a solution of (2S,4R)—N-(2-(1,4-dioxaspiro[4.5]decan-8-yloxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (130 mg, 190 umol) in THF (1.3 mL) was added HCl (1 M, 650 uL), then the mixture was stirred at 50° C., for 3 h. On completion, the reaction mixture was poured into water (1 mL), and then extracted with EtOAc (10 mL×3). The combined organic layers were washed with brine (1 mL×3), dried over Na₂SO₄, filtered and concentrated under reduced pressure to give the title compound (80 mg, 66% yield) as a yellow oil. LC-MS (ESI+) m/z 629.5 (M+H)⁺.

(1r,4r)-Ethyl 4-(tosyloxy)cyclohexanecarboxylate (Intermediate AU) and (1s,4s)-ethyl 4-(tosyloxy)cyclohexanecarboxylate (Intermediate AV)

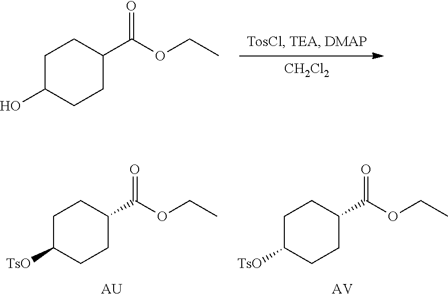

To a solution of ethyl 4-hydroxycyclohexanecarboxylate (5.00 g, 29.0 mmol, 4.67 mL) and DMAP (7.09 g, 58.1 mmol), and TEA (5.88 g, 58.1 mmol, 8.08 mL) in DCM (50 mL) was added TosCl (8.30 g, 43.6 mmol) at 0° C., then the mixture was stirred at 25° C., for 5 h. On completion, the reaction mixture was quenched with NaHCO; (20 mL) at 0° C., and then diluted with DCM (20 mL) and extracted with DCM (20 mL×3). The combined organic layers were washed with brine (15 mL×2), dried over Na₂SO₄, filtered and concentrated under reduced pressure to give a residue. The residue was purified by silica gel column chromatography (Petroleum ether/Ethyl acetate=20:1 to 7:1) to give (Ir, 4r)-ethyl 4-(tosyloxy)cyclohexanecarboxylate (3.60 g, 38% yield) as an orange solid (¹H NMR (400 MHZ, DMSO-d₆) δ 7.79 (br d, J=8.0 Hz, 2H), 7.47 (br d, J=7.6 Hz, 2H), 4.50-4.39 (m, 1H), 4.01 (q, J=6.8 Hz, 2H), 2.42 (s, 3H), 2.33-2.24 (m, 1H), 1.88-1.74 (m, 4H), 1.53-1.35 (m, 4H), 1.14 (br t, J=7.2 Hz, 3H)) and (1s,4s)-ethyl 4-(tosyloxy)cyclohexanecarboxylate (3.03 g, 32% yield) as an orange solid (¹H NMR (400 MHz, DMSO-d₆) δ 7.79 (br d, J=8.4 Hz, 2H), 7.47 (br d, J=8.0 Hz, 2H), 4.65 (br s, 1H), 4.04 (q, J=7.2 Hz, 2H), 2.42 (s, 3H), 2.36 (br s, 1H), 1.68-1.52 (m, 8H), 1.16 (t, J=7.2 Hz, 3H)).

### (1R,4s)-4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylic acid (Intermediate AW)

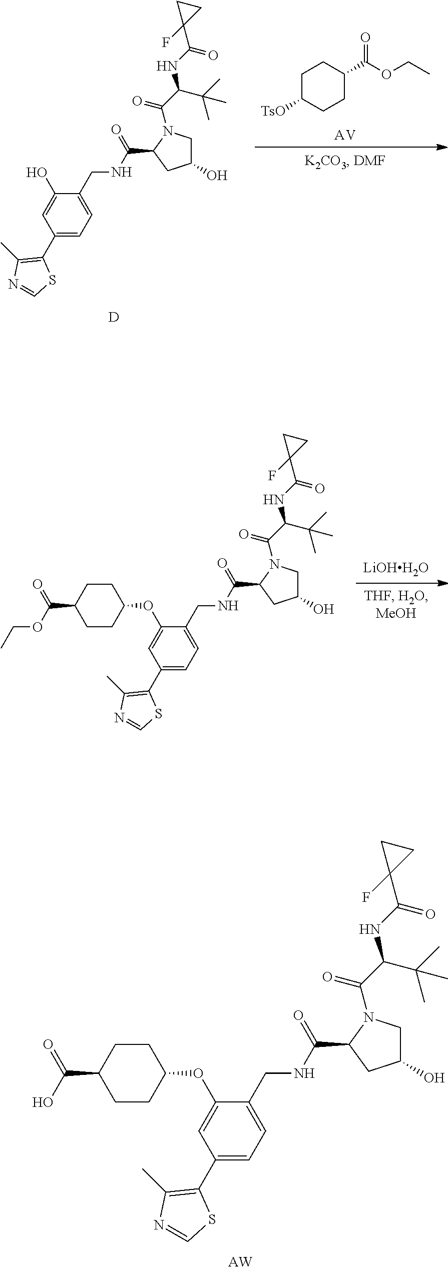

### Step 1—(1S,4r)-ethyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylate

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (300 mg, 563 umol, Intermediate D) and (1s,4s)-ethyl 4-(tosyloxy)cyclohexanecarboxylate (919 mg, 2.82 mmol, Intermediate AV) in DMF (5 mL) was added K₂CO₃ (389 mg, 2.82 mmol), then the mixture was stirred at 90° C., for 41 h. On completion, the reaction mixture was quenched with NH₄Cl (2 mL) and extracted with EtOAc (5 mL×2). The combined organic layers were dried over Na₂SO₄ and evaporated to give the title compound (500 mg) as an orange solid. LC-MS (ESI+) m/z 687.3 (M+H)⁺.

### Step 2—(1R,4s)-4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylic acid

To a solution of (1S,4r)-ethyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylate (400 mg, 454 umol) in THF (6 mL), H₂O (1.2 mL), MeOH (0.6 mL) was added LiOH·H₂O (153 mg, 3.63 mmol), then the mixture was stirred at 50° C., at 4 h. On completion, the reaction mixture was filtered and the pH of the aqueous phase was adjusted to 4˜5 by addition 2M HCl and extracted with EtOAc (5 mL×2). The combined organic layers were dried over Na₂SO₄ and evaporated. The residue was purified by silica gel column chromatography (Petroleum ether/Ethyl acetate=5:1 to 0:1) to give the title compound (250 mg, 84% yield) as an orange solid. LC-MS (ESI+) m/z 659.2 (M+H)⁺.

### (1R,4s)-4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylic acid (Intermediate AX)

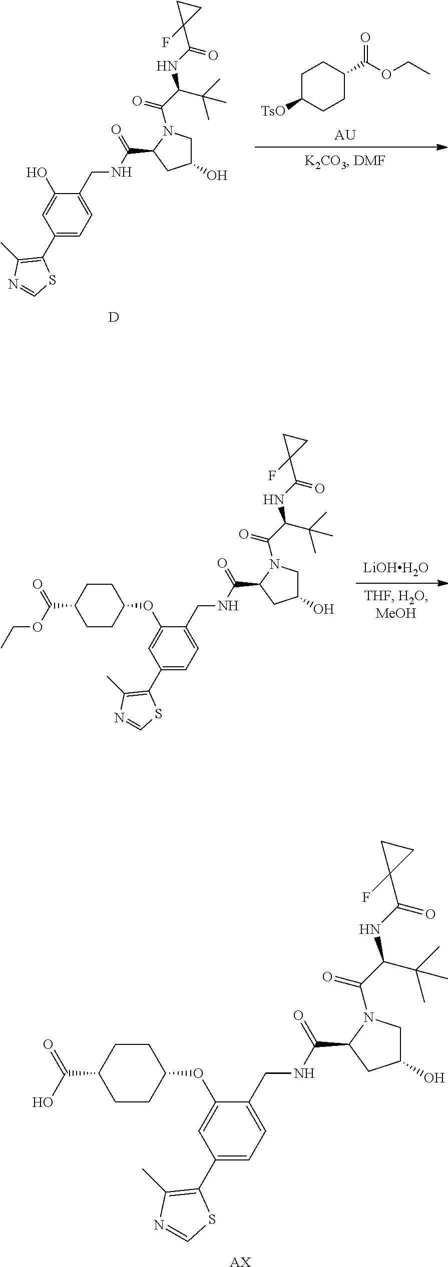

### Step 1—(1R,4s)-ethyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylate

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (300 mg, 600 umol, Intermediate D) and (1r,4r)-ethyl 4-(tosyloxy)cyclohexanecarboxylate (735 mg, 2.82 mmol, Intermediate AU) in DMF (5 mL) was added K₂CO; (389 mg, 2.82 mmol), then the mixture was stirred at 90° C., for 22 h. On completion, the reaction mixture was quenched with NH₄Cl (2 mL) and extracted with EtOAC (5 mL×2). The combined organic layers were dried over Na₂SO₄ and evaporated to give the title compound (400 mg) as an orange solid. LC-MS (ESI+) m/z 687.6 (M+H)⁺.

### Step 2 (1R,4s)-4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylic acid

To a solution of (1R,4s)-ethyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclohexanecarboxylate (300 mg, 400 umol) in THF (6 mL), H₂O (1.2 mL), and MeOH (0.6 mL) was added LiOH·H₂O (147 mg, 3.49 mmol). Then the mixture was stirred at 50° C., at 4 h. On completion, the reaction mixture was filtered and the pH of the aqueous phase was adjusted to 4˜5 by addition 2M HCl and extracted with EtOAC (5 mL×2). The combined organic layers were dried over Na₂SO₄ and evaporated. The residue was purified by silica gel column chromatography (Petroleum ether/Ethyl acetate=5:1 to 0:1) to give the title compound (287 mg, quant, yield) as an orange solid. LC-MS (ESI+) m/z 659.2 (M+H)⁺.

### (1s,3s)-methyl 3-(tosyloxy)cyclobutanecarboxylate (Intermediate AY)

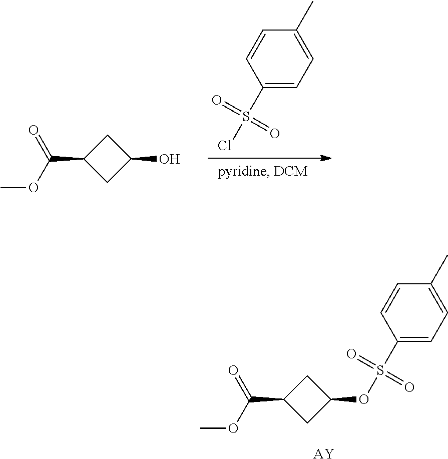

To a solution of 4-methylbenzenesulfonyl chloride (2.93 g, 15.4 mmol), (1s,3s)-methyl 3-hydroxycyclobutanecarboxylate (1.00 g, 7.68 mmol, CAS #63485-50-7) and pyridine (3.43 g, 43.4 mmol) in DCM (9.5 mL) was degassed and purged with N₂ three times at 0° C. Then the mixture was stirred at 20° C., for 16 h under N₂ atmosphere. On completion, the reaction mixture was quenched with H₂O (10 mL), and then extracted with DCM (10 mL×3). The combined organic layers were washed with brine (10 mL×3), dried over Na₂SO₄, filtered and concentrated under reduced pressure to give a residue. The residue was purified by flash silica gel chromatography (ISCOR: 20 g SepaFlash® Silica Flash Column, Eluent of 11˜33% Ethyl acetate/Petroleum ethergradient @ 60 mL/min) to give the title compound (1.90 g, 83% yield) as a red oil. ¹H NMR (400 MHZ, DMSO-d₆) δ=7.79 (d, J=8.4 Hz, 2H), 7.49 (d, J=8.0 Hz, 2H), 4.75 (quin, J=7.5 Hz, 1H), 3.58 (s, 3H), 2.73 (tt, J=7.9, 9.7 Hz, 1H), 2.43 (s, 3H), 2.42-2.37 (m, 2H), 2.21-2.13 (m, 2H).

### (1S,3r)-3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclobutanecarboxylic acid (Intermediate AZ)

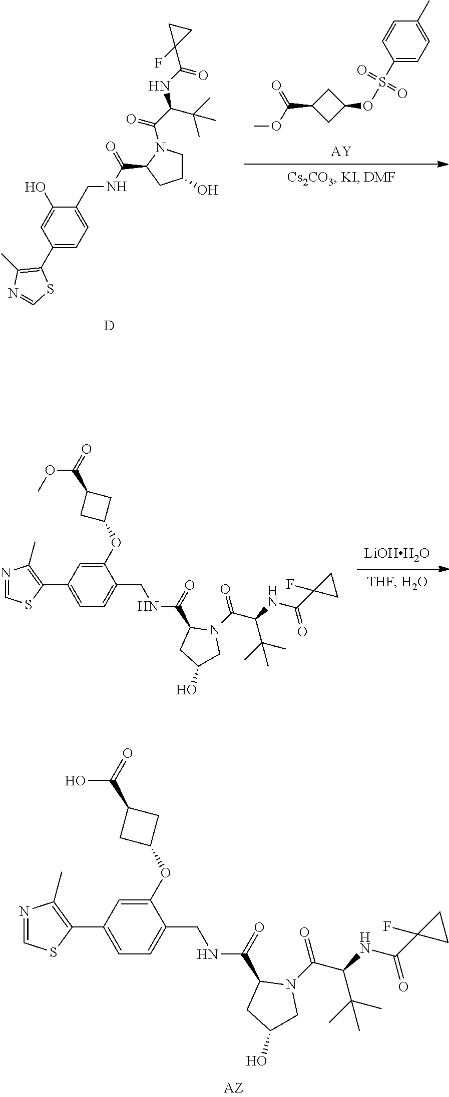

### Step 1—(1S,3r)-methyl 3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclobutanecarboxylate

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (200 mg, 0.4 mmol, Intermediate D), (1s,3s)-methyl 3-(tosyloxy)cyclobutanecarboxylate (326 mg, 1.09 mmol, Intermediate AY), K₂CO; (104 mg, 0.751 mmol) and KI (6.23 mg, 0.0376 mmol) in DMF (1 mL) was degassed and purged with N₂ three times. Then the mixture was stirred at 80° C., for 12 h under N₂ atmosphere. On completion, the reaction mixture was quenched with NH₄Cl (1 mL), and then extracted with EtOAc (1 mL×3). The combined organic layers were washed with brine (1 mL×3), dried over Na₂SO₄, filtered and concentrated under reduced pressure to give a residue. The residue was purified by flash silica gel chromatography (ISCOR: 3 g SepaFlash® Silica Flash Column, Eluent of 100% Ethyl acetate/Petroleum ether gradient (60 mL/min) to give the title compound (200 mg, 70% yield) as a yellow solid. ¹H NMR (400 MHZ, DMSO-d₆) δ=8.99 (s, 1H), 8.50 (t, J=6.0 Hz, 1H), 7.42 (d, J=8.0 Hz, 1H), 7.29 (br dd, J=2.4, 9.2 Hz, 1H), 6.97 (dd, J=1.6, 8.0 Hz, 1H), 6.75 (d, J=1.6 Hz, 1H), 5.18 (d, J=3.6 Hz, 1H), 4.93 (t, J=6.0 Hz, 1H), 4.60 (d, J=9.2 Hz, 1H), 4.51 (t, J=8.0 Hz, 1H), 4.36 (br s, 1H), 4.26 (br dd, J=6.0, 9.6 Hz, 2H), 3.66 (s, 3H), 3.64-3.60 (m, 2H), 3.29-3.20 (m, 2H), 2.68 (br dd, J=2.0, 3.6 Hz, 2H), 2.46 (br s, 1H), 2.45 (s, 3H), 2.10-2.04 (m, 1H), 1.94-1.90 (m, 1H), 1.41-1.38 (m, 1H), 1.36-1.34 (m, 1H), 1.23 (br d, J=3.2 Hz, 1H), 1.21 (br d, J=3.2 Hz, 1H), 0.96 (s, 9H), 0.95 (br s, 1H).

### Step 2—(1S,3r)-3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclobutanecarboxylic acid

To a solution of (1S,3r)-methyl 3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)cyclobutanecarboxylate (100 mg, 0.2 mmol) and LiOH·H₂O (13.0 mg, 0.310 mmol) in THF (0.8 mL) and H₂O (0.2 mL) was stirred at 20° C., for 8 h under N₂ atmosphere. On completion, the reaction mixture was quenched with HCl (2M) the pH was adjusted to 3˜4. Then to the mixture was added H₂O (2 mL), and extracted with EtOAc (2 mL×3). The combined organic layers were washed with brine (2 mL×2), dried over Na₂SO₄, filtered and concentrated under reduced pressure to give the title compound (90.0 mg) as a white solid. LC-MS (ESI+) m/z 631.5 (M+H)⁺.

### (R)-N-((4-((4-(4-(2-azaspiro[3.5]nonane-7-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1,1′-biphenyl]-2-yl)methyl)piperazin-1-yl)benzamide (Intermediate BA)

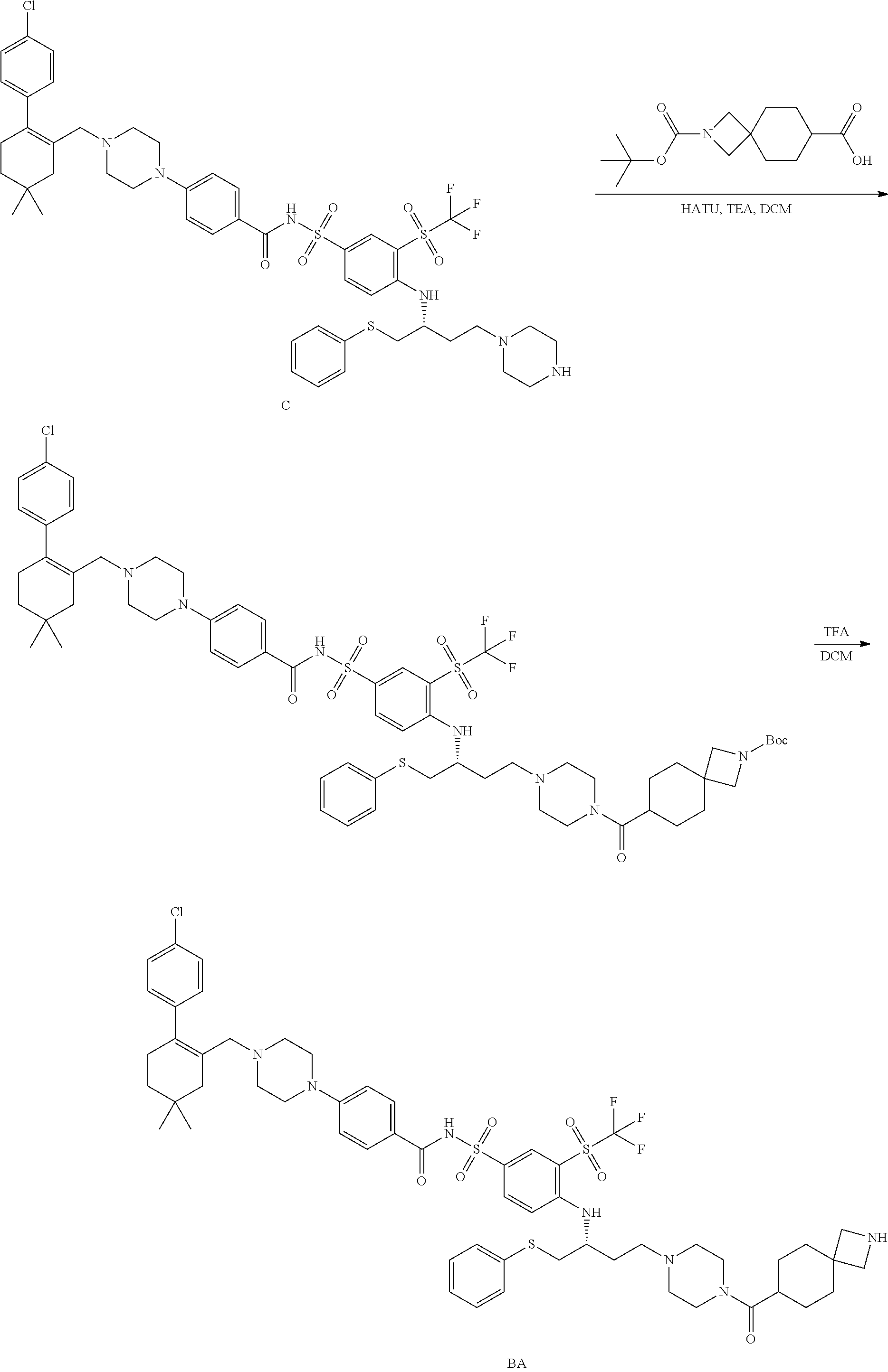

### Step 1—(R)-tert-butyl 7-(4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)-2-azaspiro[3.5]nonane-2-carboxylate

To a solution of (R)-4-(4-((4′-chloro-4,4-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (100 mg, 103 umol, Intermediate C), 2-tert-butoxycarbonyl-2-azaspiro[3.5]nonane-7-carboxylic acid (27.7 mg, 103 umol, CAS #1363381-18-3) and TEA (52.0 mg, 514 umol) in DCM (1 mL) was added HATU (41.0 mg, 108 umol), then the mixture was stirred at 25° C., for 1 h. On completion, the reaction mixture was quenched with H₂O (2 mL) at 0° C., and extracted with ethyl acetate (2 mL×3). The combined organic layers were washed with brine (2 mL×3), dried over Na₂SO₄, filtered and concentrated. The residue was purified by column chromatography (SiO₂, Petroleum ether/Ethyl acetate=20/1-0/1, Dichloromethane:Methanol=20/1-10/1) to give the title compound (120 mg, 95% yield) as a yellow oil. LC-MS (ESI+) m/z 1225.0 (M+H)⁺.

### Step 2—(R)-N-((4-((4-(4-(2-azaspiro[3.5]nonane-7-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1, l′-biphenyl]-2-yl)methyl)piperazin-1-yl)benzamide

The solution of (R)-tert-butyl 7-(4-(3-((4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)-2-azaspiro[3.5]nonane-2-carboxylate (100 mg, 80 umol) in DCM (1 mL) was added TFA (0.2 mL), then the mixture was stirred at 25° C., for 1 h. On completion, the reaction mixture was concentrated to give the title compound (55 mg, 60% yield) as a yellow oil. LC-MS (ESI+) m/z 1124.7 (M+H)⁺.

Phenyl N-[(1S)-1-[(2S,4R)-4-hydroxy-2-[(1S)-1-[4-(4-methylthiazol-5-yl)phenyl]ethyl]carbamoyl]pyrrolidine-1-carbonyl]-2,2-dimethyl-propyl]carbamate (Intermediate BB)

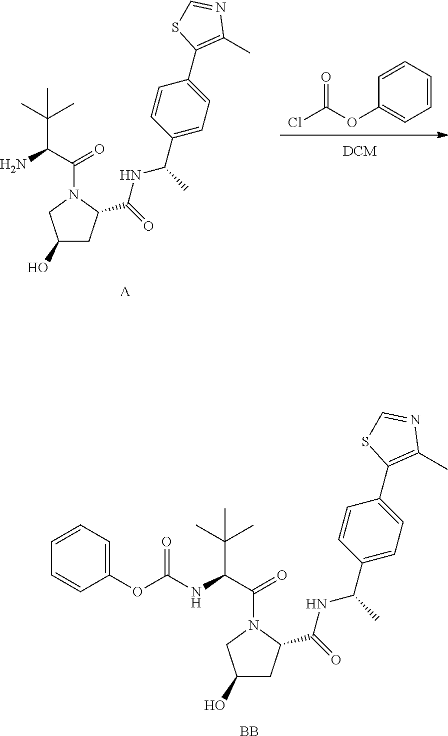

### Step 1—Phenyl ((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamate

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (3 g, 6.24 mmol, HCl, Intermediate A), phenyl carbonochloridate (1.17 g, 7.48 mmol, 937 uL) in DCM (30 mL) was added TEA (1.89 g, 18.71 mmol, 2.60 mL). The mixture was stirred at 25° C., for 2 h. On completion, the mixture was filtered. The filtrate was washed with H₂O (20 mL×2), then with brine (20 mL), dried over anhydrous Na₂SO₄, filtered and concentrated under reduced pressure to give the title compound (2 g, 57% yield) as a white solid. LC-MS (ESI+) m/z 565.3 (M+H)⁺.

### 1-(Cyclohexylmethyl)-5-methyl-4-(4,4,5,5-tetramethyl-1,3,2-dioxaborolan-2-yl)-1H-pyrazole Intermediate BC)

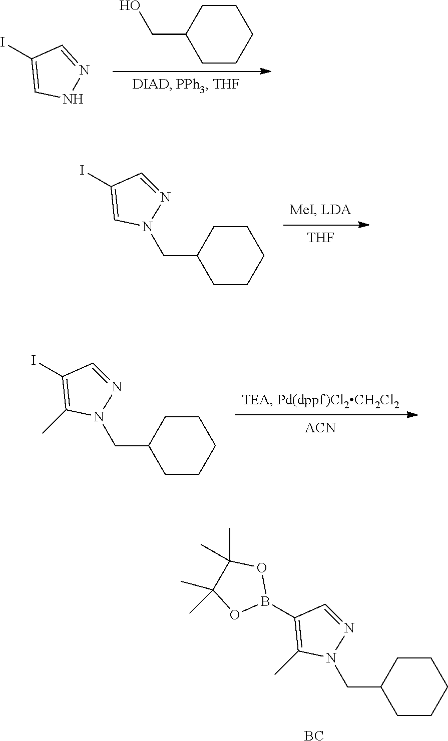

### Step 1—1-(Cyclohexylmethyl)-4-iodo-1H-pyrazole

To a solution of cyclohexylmethanol (40 g, 350 mmol, 43.0 mL), 4-iodo-1H-pyrazole (68.0 g, 350 mmol) and PPh; (137 g, 525 mmol) in THF (300 mL) was added a solution of DIAD (106 g, 525 mmol, 102.17 mL) in THF (300 mL) at 0° C., under N₂, then the mixture was stirred at 25° C., for 12 h. On completion, the reaction mixture was quenched with H₂O (600 mL) at 20° C., and extracted with EtOAc (600 mL×3). The combined organic layers were washed with brine (600 mL×2), dried over Na₂SO₄, filtered and concentrated under reduced pressure to give a residue. The residue was purified by column chromatography (SiO₂, Petroleum ether/Ethyl acetate=I/O to 10/1) to give the title compound (43 g, 42% yield) as yellow solid. LC-MS (ESI+) m/z 290.8 (M+H)⁺.

### Step 2—1-(cyclohexylmethyl)-4-iodo-5-methyl-1H-pyrazole

To a solution of 1-(cyclohexylmethyl)-4-iodo-1H-pyrazole (12 g, 41 mmol) in THF (200 mL) was added LDA (2 M, 33.09 mL) at −78° C., under N₂, the mixture was stirred at −78° C., for 0.5 h, then Mel (8.81 g, 62.0 mmol, 3.86 mL) was added dropwise, and the mixture was stirred at 25° C., for 12 h under N₂. On completion, the mixture was quenched with aq. NH₄Cl 400 mL, and extracted with EtOAc (300 mL×3). The combined organic layers were washed with brine (200 mL×2), dried over Na₂SO₄, filtered and concentrated under reduced pressure to give a residue. The residue was purified by flash silica gel chromatography (ISCOR: 120 g SepaFlash® Silica Flash Column, Eluent of 0˜10% Ethyl acetate/Petroleum ethergradient @ 100 mL/min) to give the title compound (11.5 g, 91% yield) as white solid. LC-MS (ESI+) m/z 305.2 (M+H)⁺.

### Step 3—1-(cyclohexylmethyl)-5-methyl-4-(4,4,5,5-tetramethyl-1,3,2-dioxaborolan-2-yl)-1H-pyrazole

To a solution of 1-(cyclohexylmethyl)-4-iodo-5-methyl-1H-pyrazole (11 g, 36 mmol), 4,4,5,5-tetramethyl-1,3,2-dioxaborolane (13.8 g, 108 mmol, 15.74 mL) and TEA (10.9 g, 108 mmol, 15.1 mL) in ACN (115 mL) was added Pd (dppf) Cl₂·CH₂Cl₂ (2.95 g, 3.62 mmol), then the mixture was stirred at 80° C. for 2 h under N₂ atmosphere. On completion, the reaction mixture was quenched with H₂O (100 mL) at 20° C., and extracted with EtOAc (100 mL×3). The combined organic layers were washed with brine (100 mL×2), dried over Na₂SO₄, filtered and concentrated under reduced pressure to give a residue. The residue was purified by flash silica gel chromatography (ISCOR: 330 g SepaFlash® Silica Flash Column, Eluent of 0˜20% Ethyl acetate/Petroleum ethergradient (@ 100 mL/min) to give the title compound (8 g, 73% yield) as brown solid. ¹H NMR (400 MHZ, DMSO-d₆) δ=7.44 (s, 1H), 3.83 (d, J=7.2 Hz, 2H), 2.35 (s, 3H), 1.78 (ddd, J=3.6, 7.6, 11.2 Hz, 1H), 1.68-1.57 (m, 3H), 1.47 (br d, J=11.6 Hz, 2H), 1.24 (s, 12H), 1.17-1.11 (m, 3H), 0.94 (br d, J=12.0 Hz, 2H).

### 5-(Benzyloxy)-2-(tert-butoxycarbonyl)-1,2,3,4-tetrahydroisoquinoline-8-carboxylic acid carbamate (Intermediate BD)

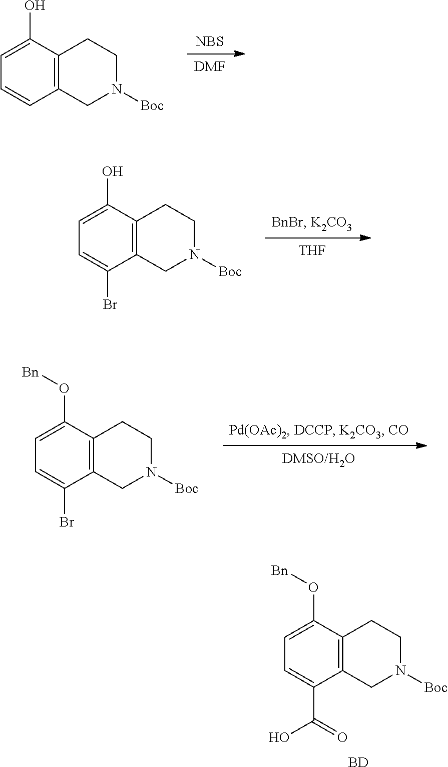

### Step 1—Tert-butyl 8-bromo-5-hydroxy-3,4-dihydroisoquinoline-2 (1H)-carboxylate

To a solution of tert-butyl 5-hydroxy-3,4-dihydro-1H-isoquinoline-2-carboxylate (50 g, 200 mmol, CAS #216064-48-1) in DMF (750 mL) was added NBS (36.0 g, 202 mmol), the mixture was stirred at 25° C., for 12 h. On completion, the mixture was quenched with water (1000 mL) and extracted with EtOAc (1000 mL×3). The combined organic layers was washed with brine (1000 mL×3) and dried over Na₂SO₄ and concentrated in vacuo to give the title compound (50 g) as yellow solid. ¹H NMR (400 MHZ, CDCl₃) ¿=7.23 (br d, J=8.4 Hz, 1H), 6.63 (br d, J=8.0 Hz, 1H), 6.54 (br s, 1H), 4.50 (s, 2H), 3.64 (t, J=6.0 Hz, 3H), 2.76 (br t, J=6.0 Hz, 3H), 1.52 (s, 10H).

### Step 2—Tert-butyl 5-(benzyloxy)-8-bromo-3,4-dihydroisoquinoline-2 (1H)-carboxylate

To a solution tert-butyl 8-bromo-5-hydroxy-3,4-dihydro-1H-isoquinoline-2-carboxylate (20 g, 60 mmol) and K₂CO₃ (16.8 g, 121 mmol) in THF (200 mL) was added bromomethylbenzene (15.6 g, 91.4 mmol), then the mixture was stirred at 60° C., for 12 h. On completion, the reaction mixture was filtered and quenched with H₂O (40 mL) at 20° C., and extracted with EtOAc (20 mL×3). The mixture was quenched by water (10 mL) and extracted with EtOAc (10 mL×3). The combined organic layers was washed with brine (10 mL×3) and dried over Na₂SO₄ and concentrated in vacuo to give the title compound (22 g) as yellow solid. ¹H NMR (400 MHZ, CDCl₃) δ=7.39-7.20 (m, 8H), 6.60 (d, J=8.8 Hz, 1H), 4.98 (s, 2H), 4.42 (s, 2H), 3.54 (br t, J=5.6 Hz, 2H), 2.74 (br s, 2H), 1.42 (s, 9H).

### Step 3—5-(Benzyloxy)-2-(tert-butoxycarbonyl)-1,2,3,4-tetrahydroisoquinoline-8-carboxylic acid

A solution of tert-butyl 5-benzyloxy-8-bromo-3,4-dihydro-1H-isoquinoline-2-carboxylate (5 g, 10 mmol) and dicyclohexyl (3-dicyclohexylphosphaniumylpropyl)phosphonium: ditetrafluoroborate (731 mg, 1.20 mmol), Pd (OAc) 2 (268 mg, 1.20 mmol), and K₂CO; (3.30 g, 23.9 mmol) in DMSO (50 mL) and H₂O (10 mL) was stirred at 100° C., for 12 h under CO (15 psi). On completion, the reaction mixture was filtered and quenched with H₂O (40 mL) at 20° C., then extracted with EtOAc (20 mL×3). The combined organic layers were washed with brine (40 mL×5), dried over Na₂SO₄, filtered and concentrated under reduced pressure to give a residue. The residue was purified by flash silica gel chromatography (Petroleum ether/Ethyl acetate=5/1 to 0/1) to give the title compound (2.5 g, 55% yield) as yellow solid. ¹H NMR (400 MHZ, DMSO-d₆) δ=7.80 (d, J=8.8 Hz, 1H), 7.46-7.40 (m, 2H), 7.36 (t, J=7.2 Hz, 2H), 7.32-7.27 (m, 1H), 6.99 (d, J=8.8 Hz, 1H), 5.16 (s, 2H), 4.81 (br s, 2H), 3.59-3.45 (m, 2H), 2.67 (s, 2H), 1.36 (s, 9H).

### Ethyl 6-(5-(2-aminoethoxy)-8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinate (Intermediate BE)

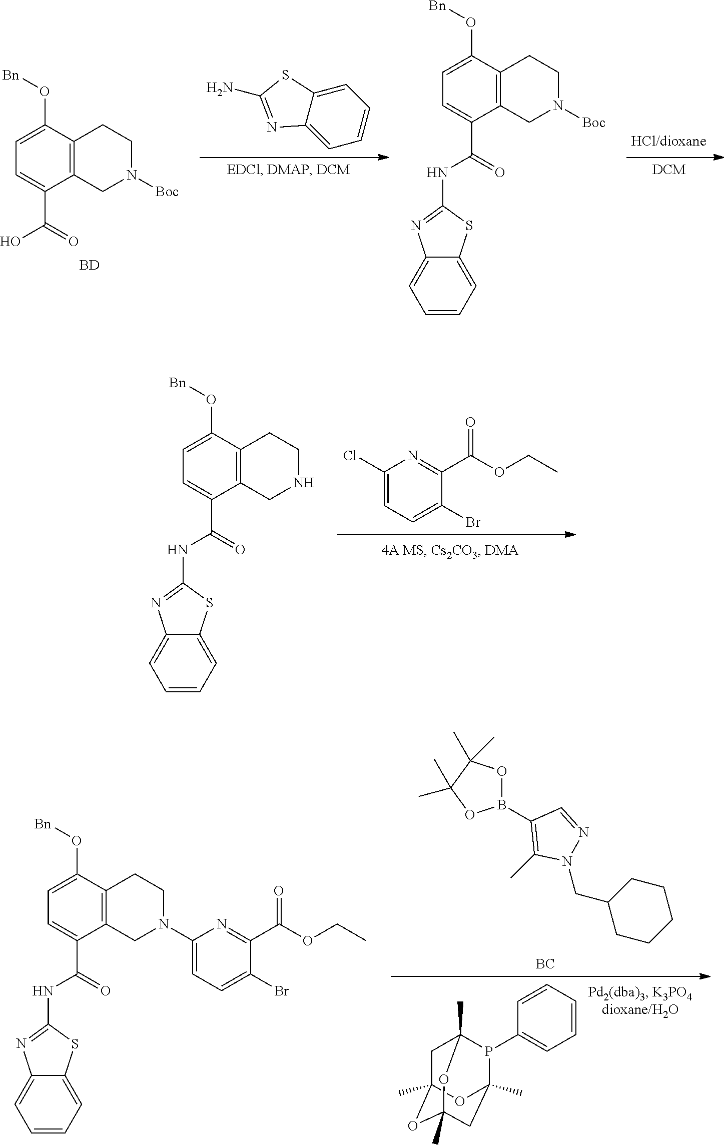

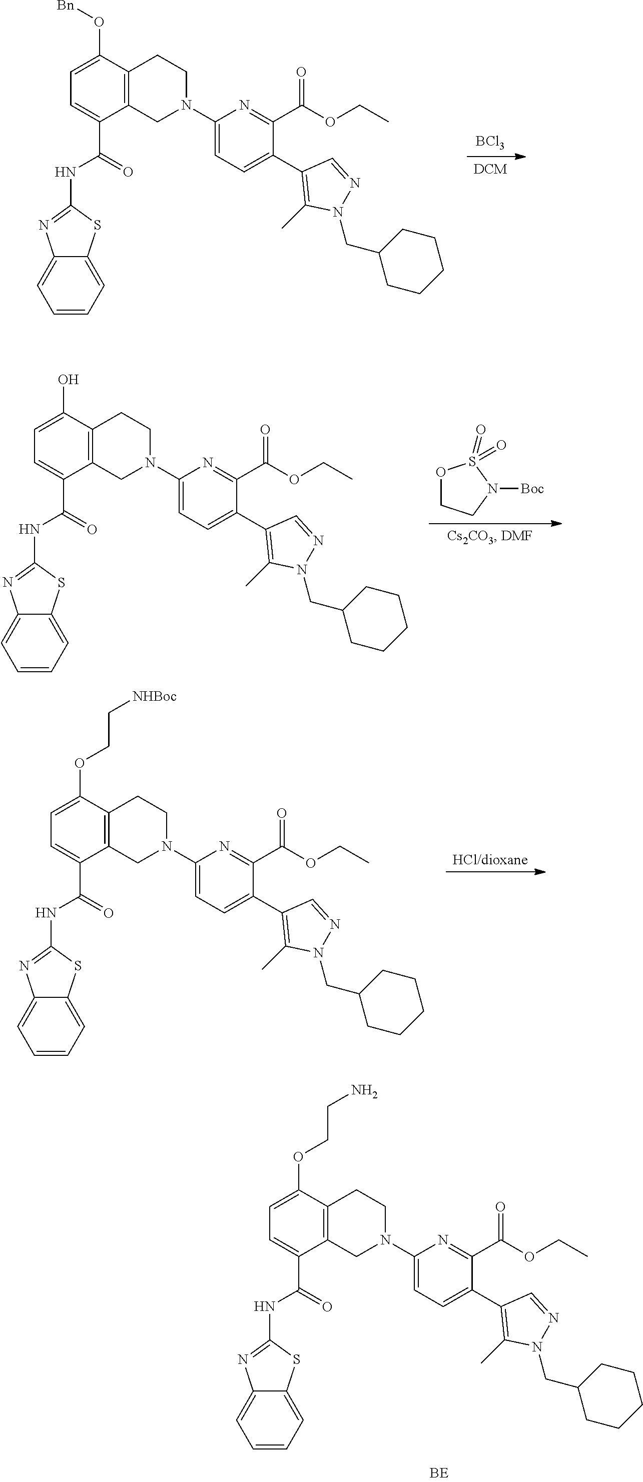

### Step 1—Tert-butyl 8-(benzo]d]thiazol-2-ylcarbamoyl)-5-(benzyloxy)-3,4-dihydroisoquinoline-2 (1H)-carboxylate

To a solution of 5-benzyloxy-2-tert-butoxycarbonyl-3,4-dihydro-IH-isoquinoline-8-carboxylic acid (2.2 g, 5.7 mmol. Intermediate BD) and 1,3-benzothiazol-2-amine (1.29 g, 8.61 mmol) in DCM (22 mL) was added DMAP (1.40 g, 11.4 mmol) and EDCI (2.20 g, 11.4 mmol), the mixture was then stirred at 25° C., for 12 h. On completion, the reaction mixture was concentrated under reduced pressure to give a residue. The residue was purified by flash silica gel chromatography (Petroleum ether/Ethyl acetate=5/1˜1/1) to give the title compound (1.2 g, 41% yield) as yellow solid. LC-MS (ESI) m/z 516.5 (M+H).

### Step 2—N-(benzo[d]thiazol-2-yl)-5-(benzyloxy)-1,2,3,4-tetrahydroisoquinoline-8-carboxamide

To a solution of tert-butyl 8-(1,3-benzothiazol-2-ylcarbamoyl)-5-benzyloxy-3,4-dihydro-1H-isoquinoline-2-carboxylate (1.2 g, 2.3 mmol) in DCM (1 mL) was added HCl/dioxane (4 M, 12.0 mL), the mixture was stirred at 25° C., for 0.5 h. On completion, the reaction mixture was concentrated under reduced pressure to give the title compound (1.7 g) as white solid.

### Step 3—Ethyl 6-(8-(benzo[d]thiazol-2-ylcarbamoyl)-5-(benzyloxy)-3,4-dihydroisoquinolin-2 (1H)-yl)-3-bromopicolinate

To a solution of N-(1,3-benzothiazol-2-yl)-5-benzyloxy-1,2,3,4-tetrahydroisoquinoline-8-carboxamide (900 mg, 2 mmol) and ethyl 3-bromo-6-chloro-pyridine-2-carboxylate (744 mg, 2.82 mmol, CAS #1065074-97-6) in DMA (10 mL) was added Cs₂CO₃ (2.12 g, 6.50 mmol), then the mixture was stirred at 100° C., for 12 h. On completion, the mixture was quenched with water (10 mL) and extracted with EtOAc (10 mL×3). The combined organic layers were washed with brine (10 mL×2), dried over Na₂SO₄, filtered and concentrated under reduced pressure to give a residue. The residue was purified by flash silica gel chromatography (Petroleum ether/Ethyl acetate=3/1) and reversed-phase HPLC (0.1% FA condition) to give the title compound (400 mg. 23% yield) as yellow solid. LC-MS (ESI) m/z 645.0 (M+H).

### Step 4—Ethyl 6-(8-(benzo[d]thiazol-2-ylcarbamoyl)-5-(benzyloxy)-3,4-dihydroisoquinolin-2 (1H)-yl)-3-(1-(cyclohexylmethyl)-5-methyl-1H-pyrazol-4-yl)picolinate

To a solution of ethyl 6-[8-(1,3-benzothiazol-2-ylcarbamoyl)-5-benzyloxy-3,4-dihydro-1H-isoquinolin-2-yl]-3-bromo-pyridine-2-carboxylate (2.4 g, 3.73 mmol) and 1-(cyclohexylmethyl)-5-methyl-4-(4,4,5,5-tetramethyl-1,3,2-dioxaborolan-2-yl) pyrazole (1.25 g, 4.10 mmol, Intermediate BC) in dioxane (24 mL) and H₂O (24 mL) was added Pd₂(dba); (341 mg. 372 umol), K₃PO₄ (2.37 g, 11.1 mmol) and (1S,3R,5R,7S)-1,3,5,7-tetramethyl-8-phenyl-2,4,6-trioxa-8-phosphatricyclo]3,3,1,1^{3,7}]decane (436 mg, 1.49 mmol), then the mixture was stirred at 100° C., for 2 h under N₂. On completion, the reaction mixture was quenched with H₂O (25 mL) at 20° C., and extracted with EtOAc (25 mL×3). The combined organic layers were washed with brine (25 mL×2), dried over Na₂SO₄, filtered and concentrated under reduced pressure to give a residue. The residue was purified by flash silica gel chromatography (Petroleum ether/Ethyl acetate=10/1˜1/1) to give the title compound (1.8 g, 65% yield) as yellow solid. ¹H NMR (400 MHz, DMSO-d₆) δ=12.69 (br s, 1H), 8.01 (d, J=8.0 Hz, 1H), 7.81-7.75 (m, 1H), 7.74-7.66 (m, 1H), 7.53-7.46 (m, 4H), 7.44-7.39 (m, 3H), 7.37-7.31 (m, 2H), 7.23-7.19 (m, 1H), 7.13-7.06 (m, 1H), 7.03 (d, J=8.9 Hz, 1H), 5.24 (s, 2H), 5.02 (s, 2H), 4.08-3.97 (m, 2H), 3.92-3.82 (m, 4H), 2.87 (br t, J=5.7 Hz, 2H), 2.12-2.06 (m, 3H), 1.84-1.73 (m, 1H), 1.70-1.57 (m, 3H), 1.20-1.10 (m, 4H), 0.97-0.92 (m, 3H).

### Step 5—Ethyl 6-(8-(benzo[d]thiazol-2-ylcarbamoyl)-5-hydroxy-3,4-dihydroisoquinolin-2 (1H)-yl)-3-(1-(cyclohexylmethyl)-5-methyl-1H-pyrazol-4-yl)picolinate

To a solution of ethyl 6-[8-(1,3-benzothiazol-2-ylcarbamoyl)-5-benzyloxy-3,4-dihydro-1H-isoquinolin-2-yl]-3-[1-(cyclohexylmethyl)-5-methyl-pyrazol-4-yl]pyridine-2-carboxylate (150 mg, 202 umol) in DCM (2 mL) was added BCI; (1 M, 4.05 mL) dropwise at 0° C., then the mixture was stirred at 25° C., for 1 h. On completion, the mixture was quenched with ice water (10 mL) and extracted with DCM (10 mL×3). The combined organic layers were washed with brine (10 mL×2), dried over Na₂SO₄, filtered and concentrated under reduced pressure to give the title compound (100 mg) as yellow solid. LC-MS (ESI) m/z 651.2 (M+H).

### Step 6-Ethyl 6-(8-(benzo[d]thiazol-2-ylcarbamoyl)-5-(2-((tert-butoxycarbonyl)amino)ethoxy)-3,4-dihydroisoquinolin-2 (1H)-yl)-3-(1-(cyclohexylmethyl)-5-methyl-1H-pyrazol-4-yl)picolinate

To a solution of tert-butyl 2,2-dioxooxathiazolidine-3-carboxylate (240 mg, 368 umol) in DMF (4 mL) was added tert-butyl 2,2-dioxooxathiazolidine-3-carboxylate (90.5 mg, 405 umol, CAS #459817-82-4) and Cs₂CO₃ (360 mg, 1.11 mmol), then the mixture was stirred at 60° C., for 12 h. On completion, the mixture was quenched with water (4 mL) and extracted with EtOAc (4 mL×3). The combined organic layers was washed with brine (4 mL×3), dried over Na₂SO₄, filtered and concentrated under reduced pressure. Then the crude product was purified by reversed-phase HPLC (0.1% NH₃·H₂O) to give the title compound (80 mg. 27% yield) as white solid. ¹H NMR (400 MHZ, DMSO-d₆) δ=10.37-9.96 (m, 1H), 7.91-7.86 (m, 1H), 7.63 (d, J=8.4 Hz, 1H), 7.57-7.48 (m, 3H), 7.42-7.31 (m, 2H), 7.02 (br d, J=8.8 Hz, 2H), 6.88-6.79 (m, 2H), 6.65 (s, 3H), 5.26 (s, 2H), 4.56 (br s, 2H), 4.10-4.02 (m, 3H), 3.93-3.84 (m, 6H), 3.47 (br d, J=5.5 Hz, 1H), 2.85-2.79 (m, 2H), 2.12 (s, 3H), 1.81 (br s, 2H), 1.72-1.61 (m, 6H), 1.59-1.50 (m, 4H), 1.42-1.41 (m, 1H), 1.24 (s, 9H), 1.12-0.91 (m, 11H).

### Step 7-Ethyl 6-(5-(2-aminoethoxy)-8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinate

A solution of ethyl 6-[8-(1,3-benzothiazol-2-ylcarbamoyl)-5-[2-(tert-butoxycarbonylamino)ethoxy]-3,4-dihydro-1H-isoquinolin-2-yl]-3-[1-(cyclohexylmethyl)-5-methyl-pyrazol-4-yl]pyridine-2-carboxylate (70 mg, 88 umol) in HCl/dioxane (4 M, 700 uL) was stirred at 25° C. for 1 h. On completion, the reaction mixture was concentrated under reduced pressure to give the title compound (70 mg) as yellow solid. LC-MS (ESI) m/z 694.2 (M+H).

### 3-(6-(8-(benzo[d]thiazol-2-ylcarbamoyl)-3,4-dihydroisoquinolin-2 (1H)-yl)-2-(tert-butoxycarbonyl)pyridin-3-yl) propanoic acid (Intermediate BF)

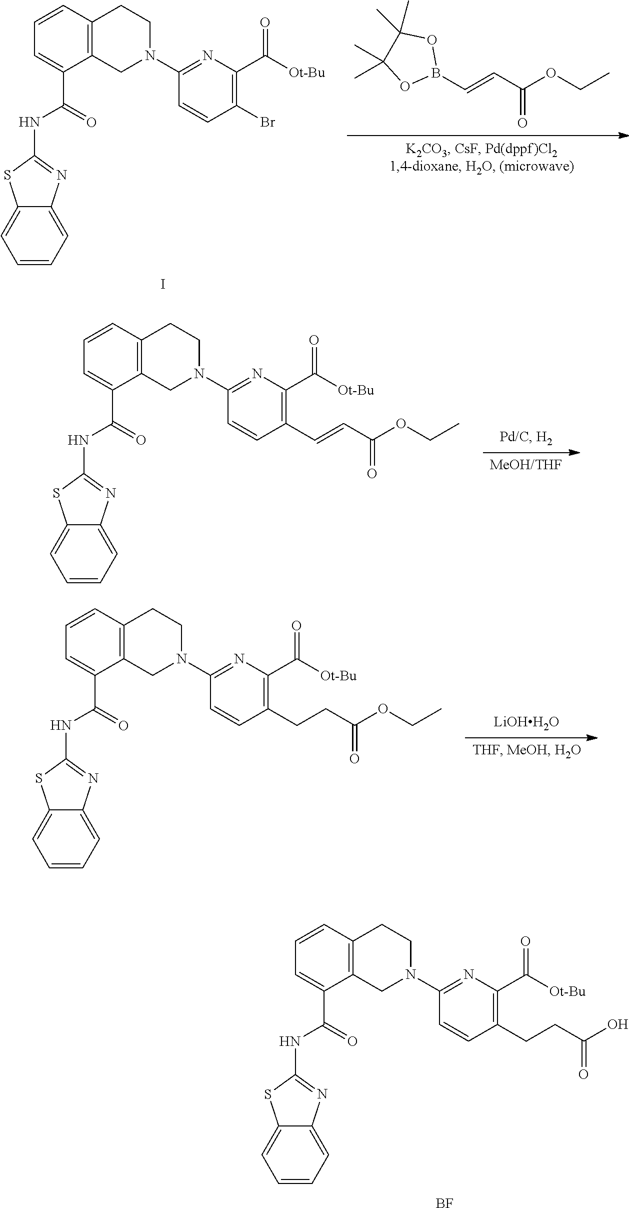

### Step 1—(E)-tert-butyl 6-(8-(benzo[d]thiazol-2-ylcarbamoyl)-3,4-dihydroisoquinolin-2 (1H)-yl)-3-(3-ethoxy-3-oxoprop-1-en-1-yl)picolinate

To a solution of tert-butyl 6-(8-(benzo[d]thiazol-2-ylcarbamoyl)-3,4-dihydroisoquinolin-2 (1H)-yl)-3-bromopicolinate (450 mg, 795 umol, Intermediate I) and (E)-ethyl 3-(4,4,5,5-tetramethyl-1,3,2-dioxaborolan-2-yl) acrylate (216 mg, 955 umol) in dioxane (4 mL) and H₂O (1.35 mL) was added CsF (242 mg, 1.59 mmol), K₂CO; (330 mg, 2.39 mmol) and Pd (dppf) Cl₂ (58.2 mg, 79.6 umol) under N₂, then the mixture was stirred at 120° C., for 30 min in a microwave. On completion, the reaction mixture was poured into water (4 mL), and then extracted with EtOAc (2 mL×3). The combined organic layer was washed with brine (2 mL×3), dried over Na₂SO₄, filtered and concentrated under reduced pressure to give a residue. The residue was purified by column chromatography (SiO₂, Petroleum ether/Ethyl acetate=10/1=1/1) to give the title compound (360 mg, 77% yield) as a yellow oil. ¹H NMR (400 MHZ, DMSO-d₆) δ=12.87 (br s, 1H), 8.10 (d, J=9.2 Hz, 1H), 8.03 (d, J=8.0 Hz, 1H), 7.78 (d, J=8.0 Hz, 1H), 7.72 (d, J=16.0 Hz, 1H), 7.59 (br d, J=7.2 Hz, 1H), 7.50-7.41 (m, 2H), 7.39-7.33 (m, 2H), 6.99 (d, J=9.6 Hz, 1H), 6.42 (d, J=15.6 Hz, 1H), 5.05 (s, 2H), 4.13 (q, J=7.2 Hz, 2H), 3.87 (br t, J=6.0 Hz, 2H), 3.02 (br t, J=6.0 Hz, 2H), 1.35 (s, 9H), 1.22 (t, J=7.2 Hz, 3H).

### Step 2—Tert-butyl 6-(8-(benzo[d]thiazol-2-ylcarbamoyl)-3,4-dihydroisoquinolin-2 (1H)-yl)-3-(3-ethoxy-3-oxopropyl)picolinate

A solution of (E)-tert-butyl 6-(8-(benzo[d]thiazol-2-ylcarbamoyl)-3,4-dihydroisoquinolin-2 (1H)-yl)-3-(3-ethoxy-3-oxoprop-1-en-1-yl)picolinate (50 mg, 85.5 umol) in MeOH (1 mL) and THF (1 mL) was added Pd/C (10 mg, 85.5 umol, 10 wt %) under H₂ (1.82 mg, 903 umol), then the mixture was stirred at 25° C., for 12 h. On completion, the reaction mixture was concentrated to give the title compound (60 mg) as a black solid. LC-MS (ESI+) m/z 587.4 (M+H)⁺.

### Step 3—3-(6-(8-(Benzo[d]thiazol-2-ylcarbamoyl)-3,4-dihydroisoquinolin-2 (1H)-yl)-2-(tert-butoxycarbonyl)pyridin-3-yl) propanoic acid

To a solution of tert-butyl 6-(8-(benzo[d]thiazol-2-ylcarbamoyl)-3,4-dihydroisoquinolin-2 (1H)-yl)-3-(3-ethoxy-3-oxopropyl)picolinate (60 mg, 100 umol) in THF (1.2 mL), MeOH (0.2 mL) and H₂O (0.2 mL) was added LiOH·H₂O (8.58 mg, 205 umol), then the mixture was stirred at 25° C., at 16 h. On completion, the reaction mixture was quenched with H₂O (1 mL) and extracted with EtOAc (1 mL×3). The aqueous phase was adjusted to pH 3˜4 with HCl, then the mixture was extracted with EtOAc (1×3). The combined organic layer was filtered and evaporated to give the title compound (25 mg, 44% yield) as a white solid. LC-MS (ESI+) m/z 559.4 (M+H)⁺.

### Ethyl 4-sulfamoylbutanoate (Intermediate BG)

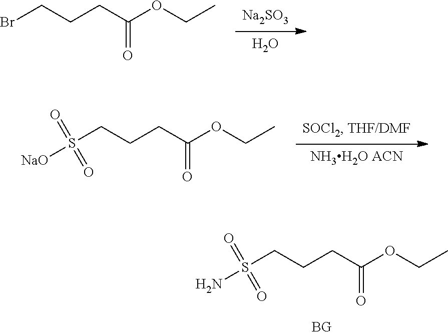

### Step 1—Sodium 4-ethoxy-4-oxobutane-1-sulfonate

To a solution of ethyl 4-bromobutanoate (25.0 g, 128 mmol) in H₂O (125 mL) was added Na₂SO₃ (21.0 g, 166 mmol), then the mixture was stirred at 120° C., for 12 h. On completion, the reaction mixture was lyophilized under reduced pressure to give the title compound (30 g) as a white solid. ¹H NMR (400 MHz, DMSO-d₆) δ=4.03 (q, J=7.2 Hz, 2H), 2.46 (t, J=7.6 Hz, 2H), 2.39 (t, J=7.6 Hz, 2H), 1.90-1.71 (m, 2H), 1.16 (t, J=7.2 Hz, 3H).

### Step 2—Ethyl 4-sulfamoylbutanoate

To a solution of sodium 4-ethoxy-4-oxobutane-1-sulfonate (15.0 g, 68.7 mmol) in THF (150 mL) and DMF (7.5 mL) was added SOCl₂ (70.7 g, 594 mmol) under N₂, then the mixture was stirred at 70° C., for 3 h. Then the solvent was concentrated in vacuo and ACN (150 mL) was added. The resulting suspension was added into NH₃·H₂O (103 g, 1.01 mol) at 0° C., and stirred for 30 min. On completion, the reaction mixture was diluted with EtOAc (150 mL). The combined organic layers were washed with H₂O (150 mL×1), brine (150 mL×1), then dried over Na₂SO₄ and evaporated. The residue was purified by flash silica gel chromatography (Ethyl acetate/Petroleum ether=0/1 to 7/10) to give the title compound (7.50 g, 45% yield) as a yellow oil. ¹H NMR (400 MHZ, DMSO-d₆) δ=6.81 (s, 2H), 4.10-4.04 (m, 2H), 3.01 (d, J=7.6 Hz, 2H), 2.46 (t, J=7.6 Hz, 2H), 1.96-1.89 (m, 2H), 1.18 (t, J=7.2 Hz, 3H).

### 1-((1s,3s)-Adamantan-1-ylmethyl)-5-methyl-4-(4,4,5-trimethyl-1,3,2-dioxaborolan-2-yl)-1H-pyrazole (Intermediate BH)

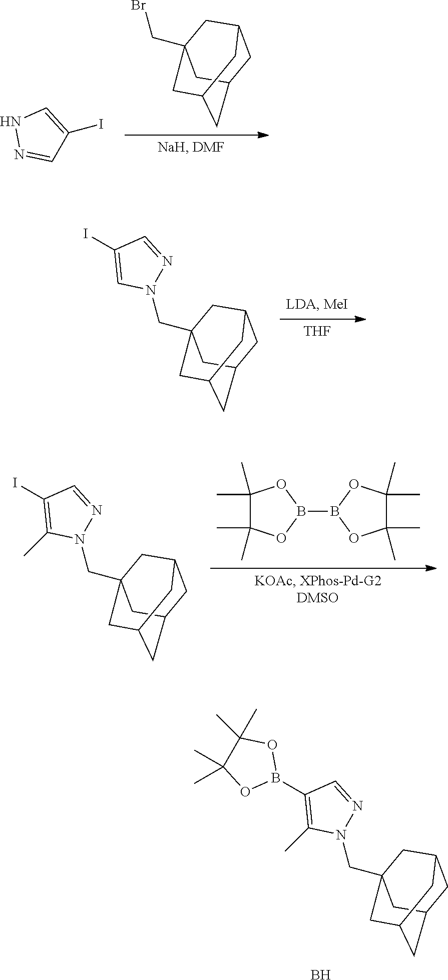

### Step 1—1-((1s,3s)-adamantan-1-ylmethyl)-4-iodo-1H-pyrazole

A mixture of 4-iodo-1H-pyrazole (16.9 g, 87.3 mmol) in DMF (200 mL) and NaH (5.24 g, 132 mmol, 60% dispersion in mineral oil) was cooled to 0° C., and stirred 0.5 h. Then (1s,3s)-1-(bromomethyl) adamantane (20.0 g, 87.3 mmol, CAS #14651-42-4) was added to the mixture and stirred at 65° C., for 12 h. On completion, the reaction mixture was partitioned between water (200 mL) and ethyl acetate (100 mL). The aqueous layer was extracted with additional ethyl acetate (250 mL×2). The combined organic layers were washed with brine, dried over Na₂SO₄, filtered, and concentrated. The residue was purified by silica gel column chromatography (Petroleum ether:Ethyl acetate=30:1˜20:1) to give the title compound (10.0 g, 33% yield) as a yellow solid. ¹H NMR (400 MHZ, DMSO-d₆) δ 7.80 (s, 1H), 7.49 (s, 1H), 3.79 (s, 2H), 1.92 (br s, 3H), 1.68-1.59 (m, 3H), 1.56-1.49 (m, 3H), 1.42 (d, J=2.0 Hz, 6H). Step 2—1-((1s,3s)-Adamantan-1-ylmethyl)-4-iodo-5-methyl-1H-pyrazole

To a solution of 1-((1s,3s)-adamantan-1-ylmethyl)-4-iodo-1H-pyrazole (9.00 g, 26.3 mmol) in THF (100 mL) was added LDA (2 M, 21.0 mL) at −70° C., and stirred at −70° C., for 0.5 h. Mel (5.6 g, 40 mmol, 2.46 mL) was added in dropwise and the mixture was stirred at −70˜ 25° C., for 12 h. On completion, the mixture was quenched with H₂O (80 mL) and extracted with EtOAc (100 mL×3). The combined organic layers was washed with brine (50 mL×3) and dried over Na₂SO₄ and concentrated in vacuo to give the title compound (9.38 mg) as a white solid. LC-MS (ESI+) m/z 357.1 (M+H)⁺.

### Step 3—1-((1s,3s)-Adamantan-1-ylmethyl)-5-methyl-4-(4,4,5-trimethyl-1,3,2-dioxaborolan-2-yl)-1H-pyrazole

To a solution of 1-((1s,3s)-adamantan-1-ylmethyl)-4-iodo-5-methyl-1H-pyrazole (9.38 g, 26.3 mmol), 4,4,4′,4′,5,5,5′,5′-octamethyl-2,2′-bi (1,3,2-dioxaborolane) (10.0 g, 39.5 mmol), XPhos-Pd-G2 (622 mg, 790 umol) in DMSO (100 mL) was added KOAc (7.75 g, 79.0 mmol) under N₂, then the mixture was stirred at 50° C., for 17 h. On completion, the reaction mixture was quenched with NH₄Cl (50 mL) and extracted with EtOAc (80 mL×2). The combined organic layers were dried over Na₂SO₄ and evaporated. The residue was purified by column chromatography (SiO₂, Petroleum ether/Ethyl acetate=30/1 to 20/1) to give the title compound (6.33 g, 55% yield) as a white solid. ¹H NMR (400 MHZ, DMSO-d₆) δ7.45 (s, 1H), 3.69 (s, 2H), 2.36 (s, 4H), 1.92 (br s, 4H), 1.67-1.60 (m, 4H), 1.57-1.47 (m, 12H).

### 3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinic acid (Intermediate BI)

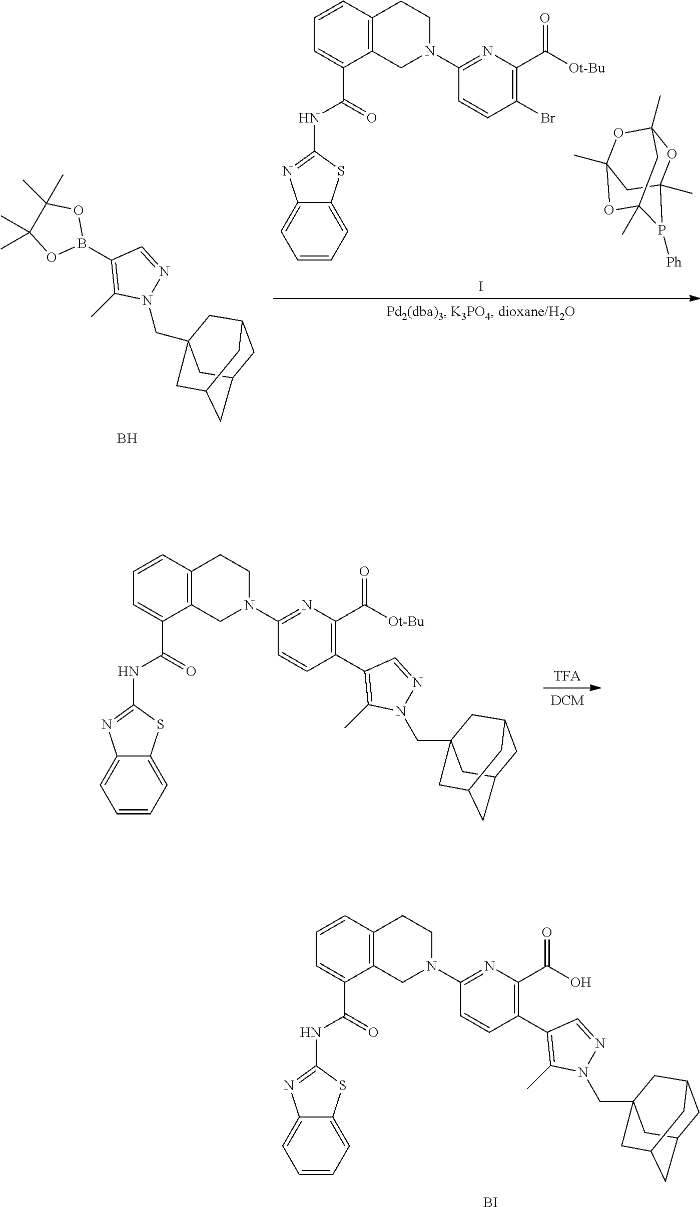

### Step 1—Tert-butyl 3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinate

To a solution of tert-butyl 6-(8-(benzo[d]thiazol-2-ylcarbamoyl)-3,4-dihydroisoquinolin-2 (1H)-yl)-3-bromopicolinate (3.17 g, 5.61 mmol, Intermediate I) and 1-(1-adamantylmethyl)-5-methyl-4-(4,4,5,5-tetramethyl-1,3,2-dioxaborolan-2-yl) pyrazole (3.00 g, 8.42 mmol, Intermediate BH) in dioxane (30 mL) and H₂O (30 mL) was added Pd₂(dba); (514 mg, 561 umol) and (3R,5S)-1,3,5,7-tetramethyl-8-phenyl-2,4,6-trioxa-8-phosphaadamantane (656 mg, 2.25 mmol) and K₃PO₄ (3.57 g, 16.8 mmol). Then the mixture was stirred at 100° C., for 12 h under N₂. On completion, the mixture was quenched with sat. NH₄Cl (20 mL) and extracted with EtOAc (50 mL×3). The combined organic layer was washed with brine (20 mL×3) and dried over Na₂SO₄ and concentrated in vacuo. The residue was purified by silica gel column chromatography (Petroleum ether/Ethyl acetate=20:1˜3:1) to afford the title compound (1.88 g, 47% yield) as an orange solid. ¹H NMR (400 MHZ, DMSO-d₆) δ12.84 (br s, 1H), 8.02 (d, J=7.6 Hz, 1H), 7.78 (d, J=8.0 Hz, 1H), 7.58 (br d, J=7.2 Hz, 1H), 7.49-7.42 (m, 3H), 7.38-7.32 (m, 2H), 7.20 (s, 1H), 6.95-6.89 (m, 1H), 4.96 (s, 2H), 3.83 (br t, J=5.8 Hz, 2H), 3.70 (s, 2H), 3.02 (br t, J=6.0 Hz, 2H), 2.08 (s, 3H), 1.99 (s, 2H), 1.91 (br s, 3H), 1.67-1.60 (m, 4H), 1.57-1.50 (m, 9H), 1.18-1.15 (m, 9H).

### Step 2—3-(1-((1s,3s)-Adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinic acid

A mixture of tert-butyl 3-(1-((1s, 3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinate (2.07 g, 2.90 mmol) and DCM (20 mL) in TFA (10 mL) was stirred at 20° C., for 12 h. On completion, the solution was concentrated in vacuo. The mixture was further purification by pre-HPLC (column: YMC Triart C18 250*50 mm*7 um: mobile phase: [water (FA)-ACN]: B %: 65%-90%, 20 min) to give the title compound (1.30 g, 66% yield) as a white solid. LC-MS (ESI⁺) m/z 659.2 (M+H)⁺: ¹H NMR (400 MHZ, DMSO-d₆) δ=13.03-12.58 (m, 1H), 8.03 (d, J=8.0 Hz, 1H), 7.79 (d, J=8.0 Hz, 1H), 7.61 (d, J=7.6 Hz, 1H), 7.49 (t, J=8.0 Hz, 2H), 7.46-7.42 (m, 1H), 7.39-7.32 (m, 2H), 7.27 (s, 1H), 6.94 (d, J=8.8 Hz, 1H), 4.95 (s, 2H), 3.88 (br t, J=6.0 Hz, 2H), 3.70 (s, 2H), 3.01 (br t, J=5.6 Hz, 2H), 2.10 (s, 3H), 1.92 (br s, 3H), 1.68-1.61 (m, 3H), 1.61-1.46 (m, 9H).

### 4-(N-(3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinoyl)sulfamoyl)butanoic acid (Intermediate BJ)

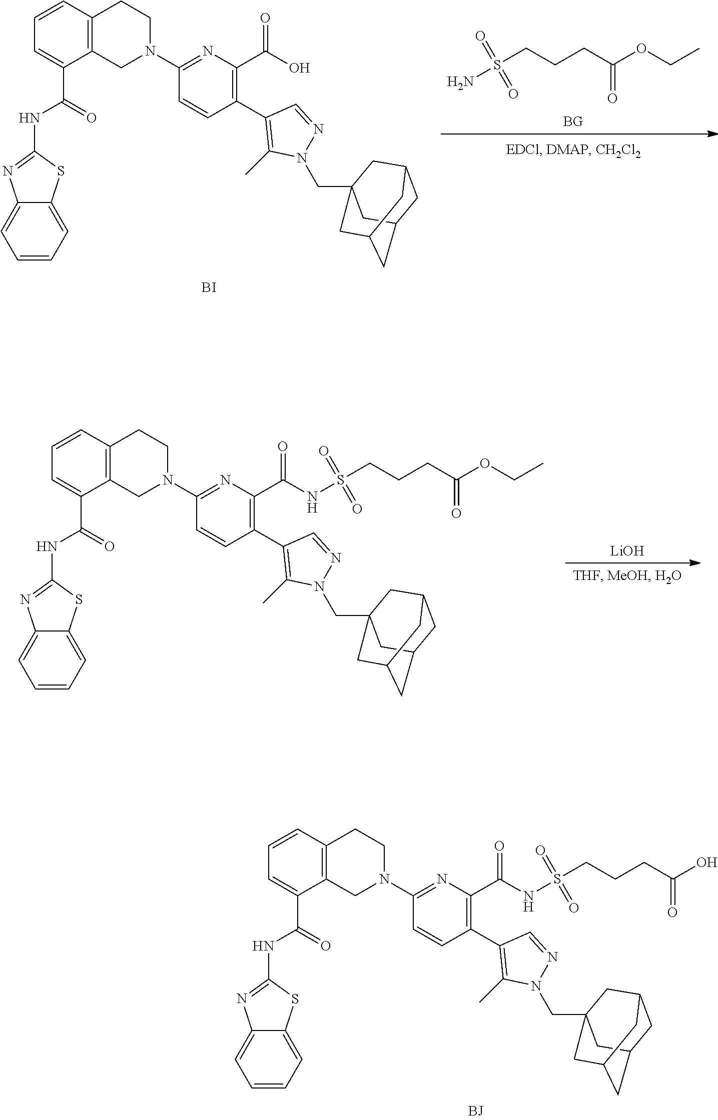

### Step 1—Ethyl 4-(N-(3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinoyl)sulfamoyl)butanoate

To a solution of 3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinic acid (200 mg, 303 umol, Intermediate BI) and ethyl 4-sulfamoylbutanoate (148 mg, 607 umol, Intermediate BG) in CH₂Cl₂ (2 mL) was added to DMAP (74.1 mg, 607 umol) and EDCI (116 mg, 607 umol), then the mixture was stirred at 25° C., for 12 h. On completion, the reaction mixture was quenched with NH₄Cl (2 mL), and then extracted with EtOAc (3 mL×3). The combined organic layers were washed with brine (3 mL×3), and dried over Na₂SO₄. The mixture was then filtered and concentrated under reduced pressure to give a residue. The residue was purified by column chromatography (SiO₂, Petroleum ether/Ethyl acetate=1/1 to 0/1) to give the title compound (150 mg, 54% yield) as a yellow solid. LC-MS (ESI⁺) m/z 836.8 (M+H)⁺.

### Step 2—4-(N-(3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinoyl)sulfamoyl)butanoic acid

To a solution of ethyl 4-(N-(3-(1-((1s,3s)-adamantan-1-ylmethyl)-5-methyl-1H-pyrazol-4-yl)-6-(8-(benzo[d]thiazol-2-ylcarbamoyl)-3,4-dihydroisoquinolin-2 (1H)-yl)picolinoyl)sulfamoyl)butanoate (150 mg, 160 umol) in THF (1 mL), MeOH (1 mL) and H₂O (1 mL) was added to LiOH·H₂O (34.2 mg, 816 umol), then the mixture was stirred at 25° C., for 12 h. On completion, the reaction mixture was acidified with HCl (IM) to pH=3˜4, and extracted with ethyl acetate (3 mL×3). The combined organic layers were washed with brine (3 mL×2), dried over Na₂SO₄, filtered and concentrated under reduced pressure to give the title compound (140 mg) as yellow solid. ¹H NMR (400 MHz, DMSO-d₆) δ=9.69 (t, J=1.2 Hz, 1H), 7.46 (8, 1H), 5.75 (8, 5H), 3.95 (s, 3H), 2.26 (8, 5H), 1.99 (8, 2H), 1.60-1.37 (m, 13H), 1.30-1.17 (m, 10H), 1.10-0.77 (m, 4H).

### (R)-N-((4-((4-(4-(3-azaspiro[5.5]undecane-9-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1,1′-biphenyl]-2-yl)methyl)piperazin-1-yl)benzamide (Intermediate BK)

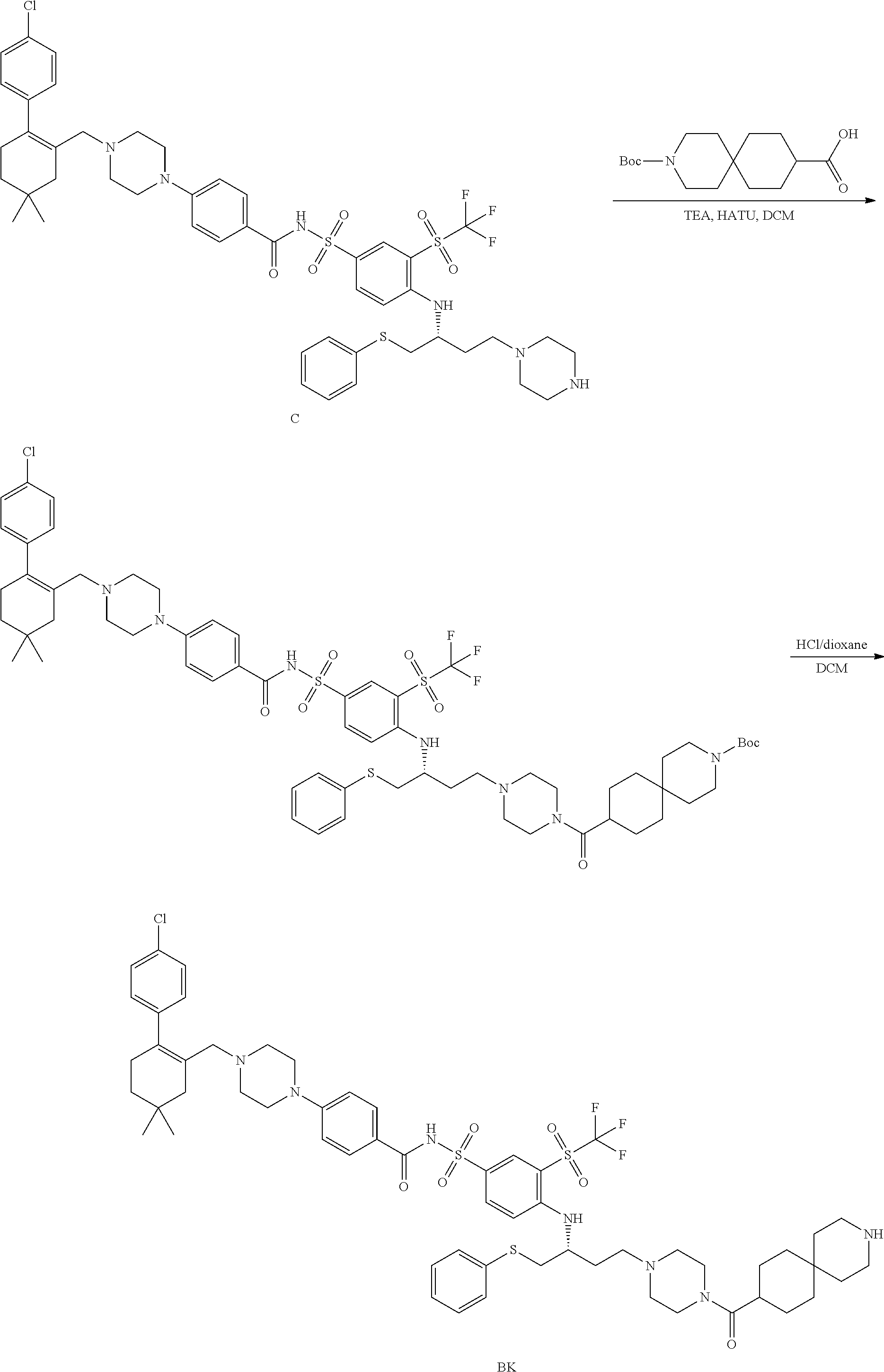

### Step 1—(R)-tert-butyl 9-(4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)-3-azaspiro[5.5]undecane-3-carboxylate

To a mixture of (R)-4-(4-((4′-chloro-4,4-dimethyl-3,4,5,6-tetrahydro-[1, l′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (100 mg, 100 umol, Intermediate C) and 3-tert-butoxycarbonyl-3-azaspiro[5.5]undecane-9-carboxylic acid (30.5 mg, 103 umol, CAS #170228-81-6) in TEA (52.0 mg, 514 umol, 71.5 uL) and DCM (1 mL) was added HATU (41.0 mg, 108 umol). The mixture was stirred at 25° C., for 1 h. On completion, the reaction mixture was quenched with H₂O (5 mL), and extracted with DCM (5 mL×3). The combined organic layers were washed with brine (5 mL×2), dried over Na₂SO₄, filtered and concentrated under reduced pressure to give a residue. The residue was purified by column chromatography (SiO₂, Petroleum ether/Ethyl acetate=20/1 to 5/1) to give the title compound (90 mg, 70% yield) as a white solid. LC-MS (ESI⁺) m/z 1251.5 (M+H)⁺.

### Step 2—(R)-N-((4-((4-(4-(3-azaspiro[5.5]undecane-9-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1, l′-biphenyl]-2-yl)methyl)piperazin-1-yl)benzamide

To a solution of (R)-N-((4-((4-(4-(3-azaspiro[5.5]undecane-9-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1, l′-biphenyl]-2-yl)methyl)piperazin-1-yl)benzamide (90 mg, 72 umol) and HCl/dioxane (8 M, 8.98 uL) in DCM (3 mL) was stirred at 25° C., for 5 min. On completion, the reaction mixture concentrated to give the title compound (57 mg) as a white solid. LC-MS (ESI⁺) m/z 1151.4 (M+H)⁺.

### Phenyl ((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamate (Intermediate BL)

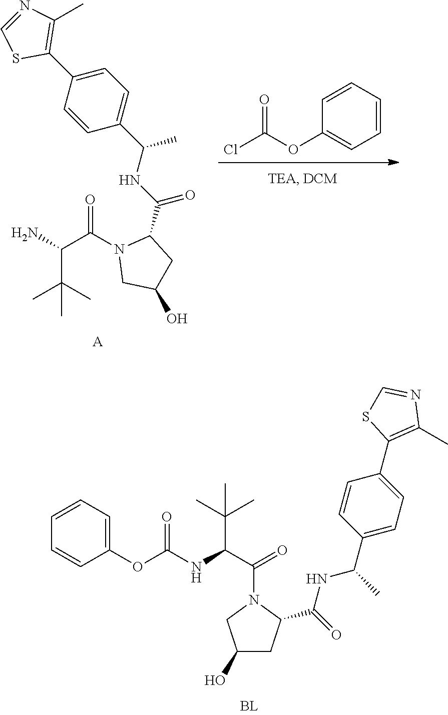

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (3 g, 6 mmol, HCl, Intermediate A), phenyl carbonochloridate (1.17 g, 7.48 mmol, 937 uL) in DCM (30 mL) was added TEA (2.60 mL, 18.71 mmol). The mixture was stirred at 25° C., for 2 hr. On completion, the mixture was filtered. The filtrate was washed with H₂O (20 mL×2), then with brine 20 mL). The filtrate was then dried over anhydrous Na₂SO₄, filtered and concentrated under reduced pressure to give the title compound (2 g, 57% yield) as a white solid. LC-MS (ESI⁺) m/z 565.3 (M+H)⁺.

### Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl) carbamate (Intermediate BM)

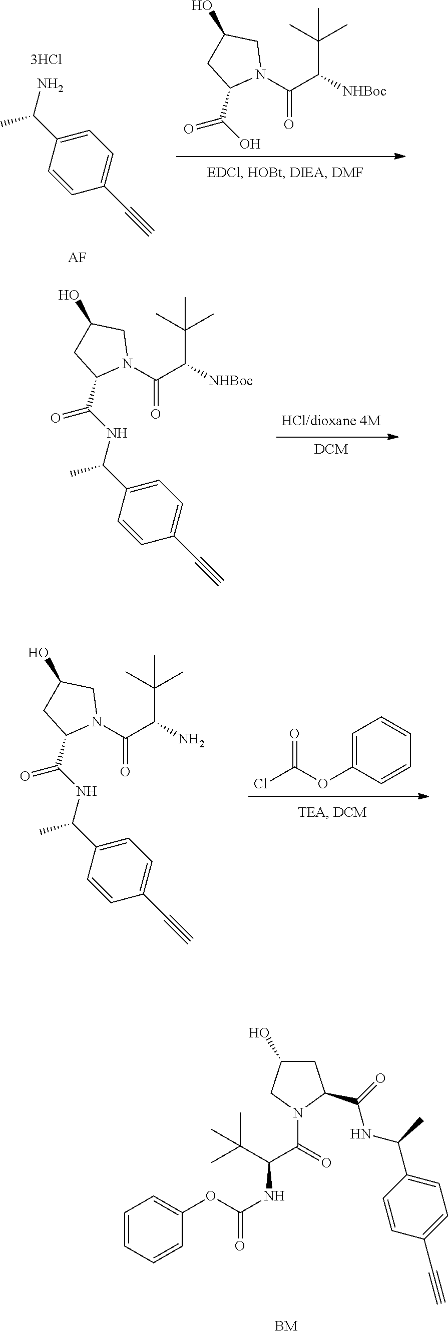

### Step 1—Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl) carbamate

To a solution of Tert-butyl(S)-(1-(4-ethynylphenyl)ethyl)carbamate (47.7 g, 187 mmol, 3 HCl, Intermediate AF) in DMF (400 mL) was added DIEA (66.7 g, 516 mmol) and the mixture was stirred at 25° C., for 0.25 hrs. Then to the mixture was added (2S,4R)-1-((S)-2- ((tert-butoxycarbonyl)amino)-3,3-dimethylbutanoyl)-4-hydroxypyrrolidine-2-carboxylic acid (43.0 g, 124 mmol), EDCI (28.7 g, 149 mmol) and HOBt (20.2 g, 149 mmol), then the mixture was stirred at 25° C., for 3 hrs. On completion, the mixture was poured into water (3.00 L), and extracted with EtOAc (1.00 L×4). The organic layer was washed with water (2.00 L×4), brine (1.50 L), dried over Na₂SO₄, filtered and concentrated. The residue was purified by silica gel chromatography eluted with Petroleum ether/Ethyl acetate (I/O to 0/1) to give the title compound (45.0 g) as a light yellow solid. LC-MS (ESI⁺) m/z 472.3 (M+H)⁺. ¹HNMR (400 MHZ, CDCl₃) δ 7.37 (br d, J=7.70 Hz, 1H), 7.27 (s, 2H), 7.08 (d, J=8.07 Hz, 2H), 5.07 (br d, J=9.05 Hz, 1H), 4.84 (quin, J=7.06 Hz, 1H), 4.52 (t, J=7.89 Hz, 1H), 4.29 (br s, 1H), 4.02 (d, J=9.17 Hz, 1H), 3.84 (br d, J=11.25 Hz, 1H), 3.24-3.35 (m, 2H), 2.86 (s, 1H), 2.19-2.33 (m, 1H), 1.94 (s, 1H), 1.18-1.30 (m, 12H), 0.83 (s, 9H).

### Step 2—(2S,4R)-1-((S)-2-amino-3,3-dimethylbutanoyl)-N-((S)-1-(4-ethynylphenyl)ethyl)-4-hydroxypyrrolidine-2-carboxamide

To a solution of Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl) carbamate (40.0 g, 84.8 mmol) in DCM (400 mL) was added HCl/dioxane (4 M, 26.0 mL) at 0-10° C., then the mixture was stirred at 10-20° C., for 16 hrs. On completion, the mixture was added dropwise into n-heptane (2.00 L). The precipitate was then filtered to give the filter cake. The compound was then dissolved in DCM (400 mL), and dissociated with basic resin (200 g) by stirring at 20° C., for 2 hrs. The mixture was filtered and concentrated to give a residue. The residue was purified by prep-HPLC (NH₃·H₂O condition) and freeze-dried to give the title compound (19.8 g, 97% yield) as a white solid. LC-MS (ESI⁺) m/z 372.1 (M+H)⁺. ¹HNMR (400 MHZ, CDCl₃) δ 7.73 (br d, J=6.40 Hz, 1H), 7.46 (d, J=8.16 Hz, 2H), 7.27 (s, 2H), 5.04 (br t, J=7.15 Hz, 1H), 4.74 (br t, J=7.72 Hz, 1H), 4.46 (br s, 1H), 3.75 (br d, J=10.92 Hz, 1H), 3.59 (br dd, J=11.04, 3.76 Hz, 1H), 3.42 (br s, 1H), 3.07 (s, 1H), 2.36 (br d, J=5.65 Hz, 1H), 2.04-2.15 (m, 1H), 1.45 (d, J=6.90 Hz, 3H), 1.02 (s, 9H).

### Step 3—Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl) carbamate

To a solution of (2S,4R)-1-[(2S)-2-amino-3,3-dimethyl-butanoyl]-N-[(1S)-1-(4-ethynylphenyl)ethyl]-4-hydroxy-pyrrolidine-2-carboxamide (50 mg, 134 umol) and phenyl carbonochloridate (21.0 mg, 134 umol) in DCM (0.5 mL) was added TEA (40.8 mg, 403 umol). The mixture was stirred at 25° C., for 2 hr. On completion, the mixture was quenched by H₂O (5 mL) and extracted with EtOAc (5 mL×3). The combined organic layers were washed with brine (5 mL×2), dried over Na₂SO₄, filtered and concentrated under reduced pressure to give the title compound (60 mg) as a white solid. LC-MS (ESI⁺) m/z 492.4 (M+H)⁺.

### (2S,4R)-N-[(4-ethynyl-2-hydroxyphenyl)methyl]-1-[(2S)-2-[(1-fluorocyclopropanecarbonyl)amino]-3,3-dimethyl-butanoyl]-4-hydroxy-pyrrolidine-2-carboxamide (Intermediate BN)

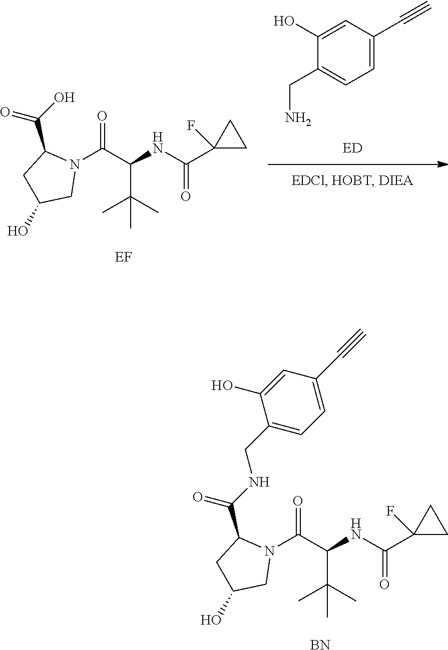

To a solution of (2S,4R)-1-((S)-2-(1-fluorocyclopropane-1-carboxamido)-3,3-dimethylbutanoyl)-4-hydroxypyrrolidine-2-carboxylic acid (50 g, 151 mmol, Intermediate EF) and 2-(aminomethyl)-5-ethynylphenol (26 g, 176 mmol, Intermediate ED) in DMF (250 mL) was added DIEA (64.9 g, 502 mmol, 87.5 mL), EDCI (35.0 g, 182 mmol) and HOBt (25.0 g, 185 mmol). The mixture was stirred at 25° C., for 3 hrs. On completion, the reaction mixture was partitioned between water (1000 mL) and ethyl acetate (1500 mL). The organic phase was separated, washed with brine (500 mL×2), dried over Na₂SO₄, filtered and concentrated under reduced pressure to give a residue. The residue was purified by flash silica gel chromatography (ISCOR; 800 g SepaFlash® Silica Flash Column, Eluent of 0˜100% Ethyl acetate/Petroleum ether gradient @ 100 mL/min) to give the title compound (45.0 g, 62% yield) as a white solid. LC-MS (ESI⁺) m/z 460.3 (M+H)⁺. ¹H NMR (400 MHZ, DMSO) & 9.83 (s, 1H), 8.51-8.48 (m, 1H), 7.29-7.21 (m, 2H), 6.85-6.81 (m, 2H), 5.17-5.16 (m, 1H), 4.59-4.57 (m, 1H), 4.49-4.45 (m, 1H), 4.33 (s, 1H), 4.20-4.16 (m, 2H), 4.14-4.04 (m, 1H), 3.63-3.59 (m, 2H), 2.08-2.03 (s, 1H), 1.91-1.85 (m, 1H), 1.39-1.35 (s, 2H), 1.34-1.20 (m, 2H), 0.95 (s, 9H).

### 2-(5-Ethynyl-2-(((2S,4R)-1-((S)-2-(1-fluorocyclopropanecarboxamido)-3,3-dimethylbutanoyl)-4-hydroxypyrrolidine-2-carboxamido)methyl)phenoxy)acetic acid (Intermediate BO)

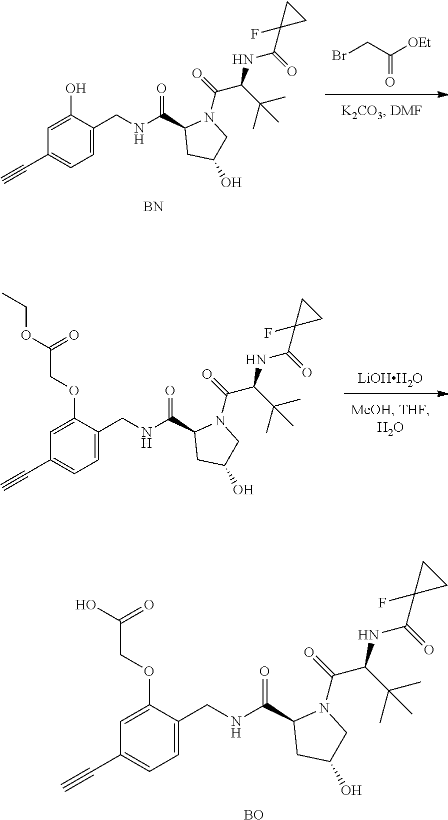

### Step 1—Ethyl 2-(5-ethynyl-2-(((2S,4R)-1-((S)-2-(1-fluorocyclopropanecarboxamido)-3,3-dimethylbutanoyl)-4-hydroxypyrrolidine-2-carboxamido)methyl)phenoxy)acetate

To a solution of ethyl 2-bromoacetate (54.5 mg, 326 umol, 36.1 uL) and (2S,4R)-N-[(4-ethynyl-2-hydroxyphenyl)methyl]-1-[(2S)-2-[(1-fluorocyclopropanecarbonyl)amino]-3,3-dimethyl-butanoyl]-4-hydroxy-pyrrolidine-2-carboxamide (100 mg, 218 umol, Intermediate BN) in DMF (1 mL) was added K₂CO; (90.2 mg, 653 umol) at 20° C., under nitrogen flow. Then the reaction was stirred at 20° C., for 5 h under nitrogen atmosphere. On completion, the reaction was poured into water (5 mL) and extracted with EtOAc (5 mL×2). The combined organic phase is washed with brine (3 mL×2), and dried over Na₂SO₄. Then the mixture was filtered to get the filtrate and concentrated to give a residue. The residue was purified by prep-TLC (petroleum ether:ethyl acetate=1:1) to give the title compound (70 mg, 54% yield) as a yellow solid. LC-MS (ESI⁺) m/z 546.3 (M+H)⁺.

### Step 2—2-(5-Ethynyl-2-(((2S,4R)-1-((S)-2-(1-fluorocyclopropanecarboxamido)-3,3-dimethylbutanoyl)-4-hydroxypyrrolidine-2-carboxamido)methyl)phenoxy)acetic acid

To a solution of ethyl 2-[5-ethynyl-2-[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]phenoxy]acetate (70 mg, 130 umol) in THF (0.5 mL), H₂O (0.5 mL) and MeOH (0.5 mL) was added LiOH·H₂O (26.9 mg, 642 umol) at 20° C., under nitrogen flow. Then the reaction was stirred at 20° C., for 10 h under nitrogen atmosphere. On completion, the reaction was poured into ice water (5 mL) and acidified by 2 N hydrochloride acid to pH=4, then extracted with EtOAc (5 mL×2). The combined organic phase is washed with brine (3 mL×2), and dried over sodium sulfate. The mixture was then filtered to get the filtrate and concentrated to give the title compound (80 mg) as a colorless oil. LC-MS (ESI⁺) m/z 518.2 (M+H)⁺.

### (2S,4R)-1-((S)-3,3-dimethyl-2-(2-(methylamino)acetamido)butanoyl)-4-hydroxy-N-((S)-1-(4-(4-methylthiazol-5-yl)phenyl)ethyl)pyrrolidine-2-carboxamide (Intermediate BP)

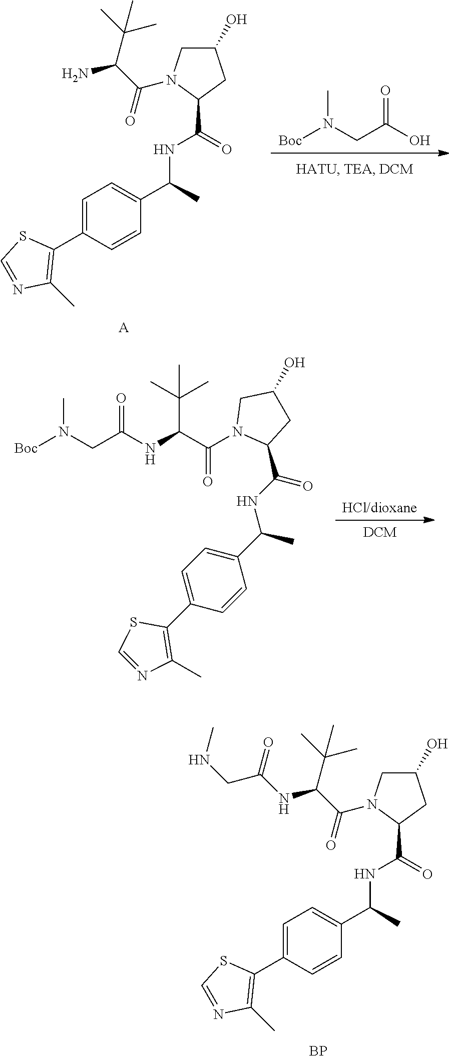

### Step 1—Tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamate

To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (200 mg, 449 umol, Intermediate A) and TEA (227 mg, 2.25 mmol) in DCM (3 mL) was added 2-((tert-butoxycarbonyl)(methyl)amino)acetic acid (85.1 mg, 449 umol, CAS #13734-36-6) and HATU (179 mg, 472 umol). Then the mixture was stirred at 25° C. for 1 h under N₂ atmosphere. On completion, the reaction mixture was filtered and then concentrated under reduced pressure. The residue was diluted with H₂O (5 mL) and extracted with EtOAc (5 mL×3). The combined organic layers were washed with brine (5 mL×2), dried over Na₂SO₄, filtered and concentrated under reduced pressure to give the title compound (270 mg) as yellow gum. LC-MS (ESI⁺) m/z 616.3 (M+H)⁺.

### Step 2—(2S,4R)-1-((S)-3,3-dimethyl-2-(2-(methylamino)acetamido)butanoyl)-4-hydroxy-N-((S)-1-(4-(4-methylthiazol-5-yl)phenyl)ethyl)pyrrolidine-2-carboxamide

A mixture of tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)(methyl)carbamate (270 mg, 438 umol) and HCl/dioxane (8 M, 54.8 uL) in DCM (2.5 mL) was degassed, and then the mixture was stirred at 25° C., for 2 h. On completion, concentrated to dryness under reduced pressure at 45° C., to give the title compound (274 mg) as green gum. LC-MS (ESI⁺) m/z 516.6 (M+H)⁺.

### Tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (Intermediate BQ)

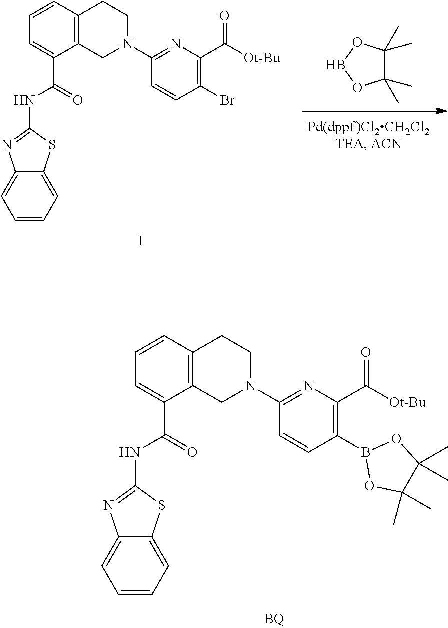

To a solution of tert-butyl 6-(8-(benzo[d]thiazol-2-ylcarbamoyl)-3,4-dihydroisoquinolin-2 (1H)-yl)-3-bromopicolinate (2.00 g, 3.54 mmol, Intermediate I) and 4,4,5,5-tetramethyl-1,3,2-dioxaborolane (6.24 g, 48.7 mmol) in ACN (20 mL) was added to TEA (1.11 g, 11.0 mmol) and Pd(dppf)Cl₂·CH₂Cl₂ (288 mg, 354 umol). Then the mixture was stirred at 80° C., for 2 h under N₂. On completion, the reaction mixture was quenched with sat. NH₄Cl (20 mL) and then extracted with EtOAc (30 mL×3). The combined organic layers were washed with brine (30 mL×3), dried over Na₂SO₄, filtered and concentrated under reduced pressure to give a residue. The residue was purified by column chromatography (SiO₂, Petroleum ether/Ethyl acetate=5/1 to 3/1) to give the title compound (1.50 g, 59% yield) as a yellow oil. ¹H NMR (400 MHZ, DMSO-d₆) δ=12.85 (s, 1H), 8.11-7.98 (m, 1H), 7.93 (s, 1H), 7.83-7.73 (m, 1H), 7.68 (d, J=8.4 Hz, 1H), 7.59-7.53 (m, 1H), 7.47 (t, J=7.6 Hz, 1H), 7.39-7.31 (m, 2H), 7.27-7.14 (m, 1H), 3.93 (s, 2H), 3.57 (s, 2H), 3.11-2.87 (m, 2H), 1.22 (s, 9H), 1.16 (s, 13H).

### 3-(1-((4-iodo-5-methyl-1H-pyrazol-1-yl)methyl)cyclohexyl)propanoic acid (Intermediate BR) (BCL-138)

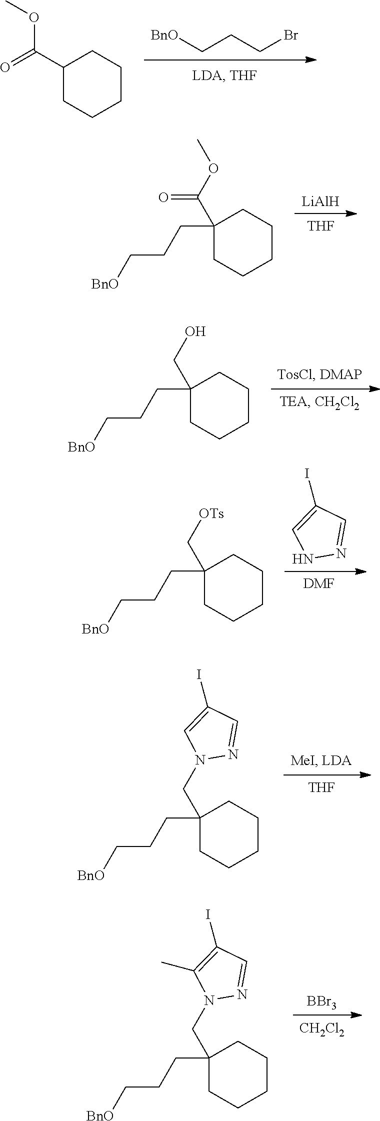

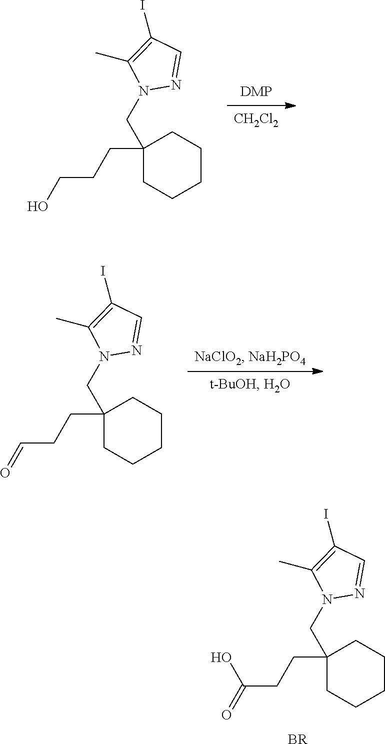

### Step 1—Methyl 1-(3-(benzyloxy)propyl)cyclohexanecarboxylate

To a solution of methyl cyclohexanecarboxylate (20.0 g, 140 mmol) in THF (200 mL) was added LDA (2 M, 105 mL) dropwise at −78° C., under N₂ and the mixture was stirred for 30 min. Then a solution of 3-bromopropoxymethylbenzene (38.6 g, 168 mmol) in THF (100 mL) was added and the mixture was stirred at 25° C., for 12 h. On completion, the reaction mixture was quenched with sat. NH₄Cl (400 mL), and extracted with EtOAc (500 mL×3). The combined organic layers were washed with brine (400 mL×3), dried over Na₂SO₄ and filtered and concentrated under reduced pressure to give a residue. The residue was purified by column chromatography (SiO₂, Petroleum ether/Ethyl acetate=10/1) to give the title compound (33.0 g, 81% yield) as a yellow oil. ¹H NMR (400 MHZ, DMSO-d₆) δ=7.38-7.24 (m, 5H), 4.41 (s, 2H), 3.60 (s, 3H), 3.36 (t, J=6.0 Hz, 2H), 1.99-1.93 (m, 2H), 1.53-1.44 (m, 5H), 1.42-1.35 (m, 2H), 1.24-1.13 (m, 5H).

### Step 2—(1-(3-(Benzyloxy)propyl)cyclohexyl)methanol

To a solution of methyl 1-(3-(benzyloxy)propyl)cyclohexanecarboxylate (15.5 g, 53.3 mmol) in THF (155 mL) was added to LiAlH₄ (4.05 g, 106 mmol) at 0° C., and the mixture was stirred at 0° C., for 2 h. On completion, the reaction mixture was quenched by dropwise addition of H₂O (4 mL), and 15% NaOH (4 mL), then H₂O (12 mL) at 0° C. The mixture was then extracted with EtOAc (50 mL×3). The combined organic layers were washed with brine (50 mL×3), dried over Na₂SO₄, filtered and concentrated under reduced pressure to give the title compound (24.5) as a yellow oil. ¹H NMR (400 MHZ. DMSO-d₆)=7.41-7.21 (m. 5H), 4.44 (s, 2H), 3.38 (t, J=6.4 Hz, 2H), 3.18 (d, J=5.2 Hz, 2H), 1.46-1.14 (m. 15H).

### Step 3—(1-(3-(benzyloxy)propyl)cyclohexyl)methyl 4-methylbenzenesulfonate

To a solution of (1-(3-(benzyloxy)propyl)cyclohexyl)methanol (21.0 g, 80.0 mmol) and DMAP (977 mg. 8.00 mmol). TEA (24.3 g, 240 mmol) in CH₂Cl₂ (210 mL) was added to TosCl (30.5 g, 160 mmol) under 0° C., then the mixture was stirred at 40° C., for 12 h. On completion, the reaction mixture was quenched with water (200 mL), and then extracted with DCM (100 mL×3). The combined organic layers were washed with brine (100 mL×3), dried over Na₂SO₄, filtered and concentrated under reduced pressure to give a residue. The residue was purified by flash silica gel chromatography (Ethyl acetate/Petroleum ether=0/1 to 3/20) to give the title compound (10.0 g, 28% yield) as a yellow oil. LC-MS (ESI) m/z 417.2 (M+H); ¹H NMR (400 MHz. DMSO-d₆) ô=7.78 (d, J=8.4 Hz, 2H), 7.45 (d, J=8.4 Hz, 2H), 7.38-7.25 (m. 5H), 4.41 (s, 2H), 3.78 (s, 2H), 3.35-3.24 (m, 2H), 2.39 (s, 3H), 1.34-1.19 (m. 14H).

### Step 4—1-((1-(3-(benzyloxy)propyl)cyclohexyl)methyl)-4-iodo-1H-pyrazole

To a solution of 4-iodo-1H-pyrazole (4.47 g, 23.0 mmol) in DMF (70 mL) was added to NaH (1.54 g, 38.4 mmol) under N₂ at 0° C., and the mixture was stirred at 0° C., for 30 min. Then (1-(3-(benzyloxy)propyl)cyclohexyl)methyl 4-methylbenzenesulfonate (8.00 g, 19.2 mmol) in DMF (30 mL) was added to the mixture and stirred at 80° C., for 16 h. On completion, the reaction mixture was quenched with sat. NH₄Cl (100 mL), and then extracted with EtOAc (100 mL×3). The combined organic layers were washed with brine (100 mL×3), dried over Na₂SO₄, filtered and concentrated under reduced pressure to give a residue. The residue was purified by flash silica gel chromatography (Ethyl acetate/Petroleum ether=0/1 to 1/20) to give the title compound (3.40 g, 40% yield) as a yellow oil. ¹H NMR (400 MHZ. DMSO-d6) δ=7.81 (s, 1H), 7.49 (s, 1H), 7.38-7.24 (m. 5H), 4.44 (s, 2H), 4.00 (s, 2H), 3.37 (t, J=6.4 Hz, 2H). 1.62-1.44 (m. 5H), 1.42-1.30 (m, 4H), 1.25-1.12 (m. 6H).

### Step 5—1-((1-(3-(benzyloxy)propyl)cyclohexyl)methyl)-4-iodo-5-methyl-1H-pyrazole

To a solution of 1-((1-(3-(benzyloxy)propyl)cyclohexyl)methyl)-4-iodo-1H-pyrazole (3.40 g. 7.76 mmol) in THF (34 mL) was added to LDA (2 M. 6.21 mL) at −78° C., and stirred at −78° C., for 30 min. Then Mel (1.65 g, 11.6 mmol) was added to the mixture and stirred at −78-25° C., for 12 h. On completion, the reaction mixture was with sat. NH₄Cl (30 mL), and then extracted with EtOAc (30 mL×3). The combined organic layers were washed with brine (30 mL×3), dried over Na₂SO₄, filtered and concentrated under reduced pressure to give the title compound (3.50 g) as a brown oil. LC-MS (ESI) m/z 453.5 (M+H); ¹H NMR (400 MHZ, DMSO-d₆) δ=7.44 (s, 1H), 7.38-7.24 (m, 5H), 4.44 (s, 2H), 3.93 (s, 2H), 3.39 (t, J=6.4 Hz, 2H), 2.25 (s, 3H), 1.50-1.43 (m, 4H), 1.34-1.20 (m, 10H).

### Step 6-3-(1-((4-iodo-5-methyl-1H-pyrazol-1-yl)methyl)cyclohexyl)propan-1-ol

To a solution of 1-((1-(3-(benzyloxy)propyl)cyclohexyl)methyl)-4-iodo-5-methyl-1H-pyrazole (4.50 g, 8.75 mmol) in CH₂Cl₂ (15 mL) was added to BCI: (31.3 g, 267 mmol), then the mixture was stirred at 25° C., for 2 h. On completion, the reaction mixture was quenched with NaHCO; (50 mL), and then extracted with EtOAc (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (2.50 g, 79% yield) as a brown oil. ¹H NMR (400 MHZ, DMSO-d₆) δ=7.45 (s, 1H), 5.75 (s, 1H), 3.92 (s, 2H), 3.35 (s, 2H), 2.26 (s, 3H), 1.49-1.35 (m, 8H), 1.29-1.21 (m, 6H).

### Step 7-3-(1-((4-iodo-5-methyl-1H-pyrazol-1-yl)methyl)cyclohexyl)propanal

To a solution of 3-(1-((4-iodo-5-methyl-1H-pyrazol-1-yl)methyl)cyclohexyl)propan-1-ol (500 mg, 1 mmol) in CH₂Cl₂ (5 mL) was added to DMP (702 mg, 1.66 mmol), then the mixture was stirred at 25° C., for 0.5 h. On completion, the reaction mixture was quenched with sat. NaHCO: (5 mL), and then extracted with EtOAc (5 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=5/1 to 1/1) to give the title compound (400 mg, 52% yield) as a yellow solid. LC-MS (ESI⁺) m/z 360.9 (M+H).

### Step 8—3-(1-((4-iodo-5-methyl-1H-pyrazol-1-yl)methyl)cyclohexyl)propanoic acid

To a solution of 3-(1-((4-iodo-5-methyl-1H-pyrazol-1-yl)methyl)cyclohexyl)propanal (400 mg. 700 umol) in t-BuOH (5 mL) and H₂O (5 mL) was added to NaH₂PO₄ (112 mg. 938 umol) and sodium: chlorite (169 mg, 1.88 mmol) at 25° C., then the mixture was stirred at 25° C., for 2 h. On completion, the reaction mixture was quenched with water (2 mL), and then extracted with EtOAc (3 mL×3). The combined organic layers were washed with brine (3 mL×3), dried over Na₂SO₄, filtered and concentrated under reduced pressure to give a residue. The crude product was purified by reversed-phase HPLC (ACN/0.1% FA condition) to give the title compound (210 mg, 75% yield) as a yellow oil. LC-MS (ESI⁺) m/z 377.1 (M+H).

### (2S,4R)-4-hydroxy-1-((S)-2-(3-(1-((4-iodo-5-methyl-1H-pyrazol-1-yl)methyl)cyclohexyl)propanamido)-3,3-dimethylbutanoyl)-N-((S)-1-(4-(4-methylthiazol-5-yl)phenyl)ethyl)pyrrolidine-2-carboxamide (Intermediate BS)

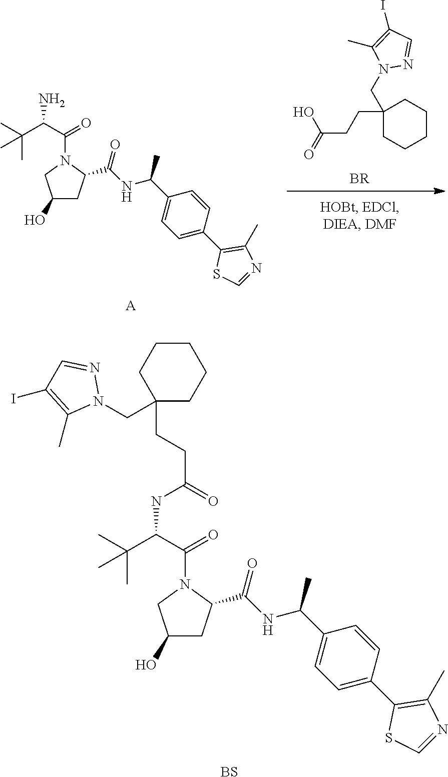

To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (212 mg, 478 umol, Intermediate A) and 3-(1-((4-iodo-5-methyl-1H-pyrazol-1-yl)methyl)cyclohexyl)propanoic acid (150 mg, 398 umol, Intermediate BR) in DMF (1 mL) was added DIEA (257 mg, 1.99 mmol), HOBt (80.8 mg, 598 umol), and EDCI (114 mg, 598 umol), then the mixture was stirred at 25° C., for 1 h. On completion, the reaction mixture was quenched with sat. NH₄Cl (2 mL), and then extracted with EtOAc (3 mL×3). The combined organic layers were washed with brine (3 mL×3), dried over Na₂SO₄, filtered and concentrated under reduced pressure to give a residue. The residue was purified by column chromatography (SiO₂, Petroleum ether/Ethyl acetate=1/1 to 0/1) to give the title compound (210 mg, 44% yield) as a yellow solid. LC-MS (ESI⁺) m/z 803.5 (M+H)⁺.

### Methyl 2-(8-(benzo[d]thiazol-2-ylcarbamoyl)-3,4-dihydroisoquinolin-2 (1H)-yl)-5-(3-hydroxypropyl)thiazole-4-carboxylate (Intermediate BT)

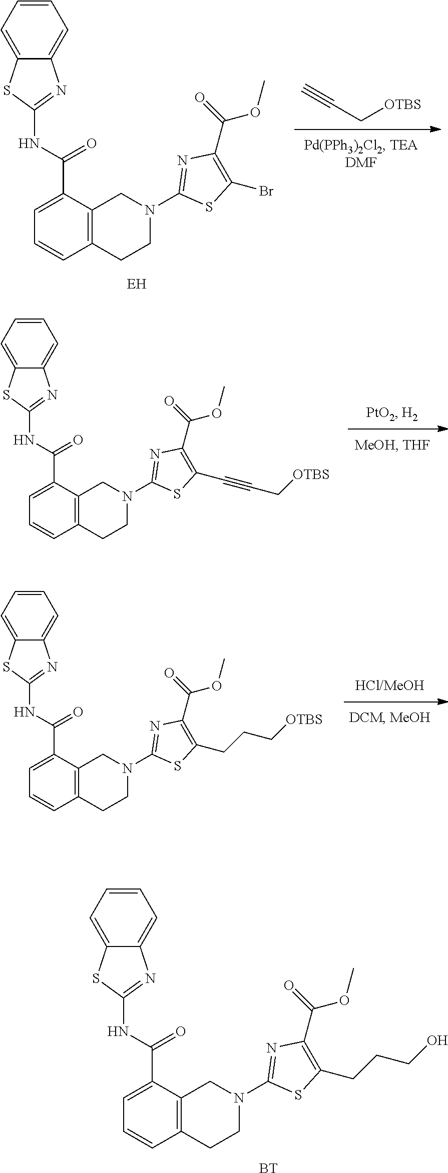

### Step 1—Methyl 2-(8-(benzo[d]thiazol-2-ylcarbamoyl)-3,4-dihydroisoquinolin-2 (1H)-yl)-5-(3-((tert-butyldimethylsilyl)oxy)prop-1-yn-1-yl) thiazole-4-carboxylate

To a solution of tert-butyl-dimethyl-prop-2-ynoxy-silane (11.5 g, 68.0 mmol) and methyl 2-(8-(benzo[d]thiazol-2-ylcarbamoyl)-3,4-dihydroisoquinolin-2 (1H)-yl)-5-bromothiazole-4-carboxylate (12.0 g, 22.7 mmol, Intermediate EH) in DMF (300 mL) was added to Pd(PPh₃)₂Cl₂ (1.59 g, 2.27 mmol), Cul (863 mg, 4.53 mmol), and TEA (11.4 g, 113 mmol). Then the mixture was stirred at 75° C., for 12 h. On completion, the reaction mixture was quenched with NH₄Cl (300 mL), and then extracted with DCM (300 mL×5). The combined organic layers were washed with brine (500 mL×3), dried over Na₂SO₄, filtered and concentrated under reduced pressure to give residue. The residue was purified by flash silica gel chromatography (Ethyl acetate/Petroleum ether=0/1 to 1/1) give the title compound (20.0 g) as a yellow solid. ¹H NMR (400 MHZ, DMSO-d₆) δ=12.91 (s, 1H), 8.03 (d, J=7.6 Hz, 1H), 7.79 (d, J=8.0 Hz, 1H), 7.69 (d, J=6.4 Hz, 1H), 7.51-7.44 (m, 2H), 7.43-7.32 (m, 2H), 5.75 (s, 1H), 4.90 (s, 2H), 4.57 (s, 2H), 3.74 (s, 5H), 3.06 (t, J=5.6 Hz, 2H), 1.38 (s, 2H), 0.86 (s, 10H), 0.10 (s, 6H).

### Step 2—Methyl 2-(8-(benzo[d]thiazol-2-ylcarbamoyl)-3,4-dihydroisoquinolin-2 (1H)-yl)-5-(3-((tert-butyldimethylsilyl)oxy)propyl)thiazole-4-carboxylate

To a solution of methyl 2-(8-(benzo[d]thiazol-2-ylcarbamoyl)-3,4-dihydroisoquinolin-2 (1H)-yl)-5-(3-((tert-butyldimethylsilyl)oxy)prop-1-yn-1-yl) thiazole-4-carboxylate (20.0 g, 32.3 mmol) in THF (40 mL) and MeOH (200 mL) was added to PtO₂ (2.00 g, 8.81 mmol) under H₂, then the mixture was stirred at 25° C., for 96 h. On completion, the reaction mixture was filtered and concentrated under reduced pressure to give the title compound (13.0 g) as a black solid. ¹H NMR (400 MHZ, DMSO-d₆) δ=8.01 (d, J=7.6 Hz, 1H), 7.82-7.72 (m, 1H), 7.71-7.52 (m, 3H), 4.81 (s, 2H), 4.02 (s, 2H), 3.73 (d, J=1.6 Hz, 2H), 3.71 (s, 3H), 3.58 (t, J=6.0 Hz, 3H), 3.06-3.02 (m, 5H), 1.73 (dd, J=6.8, 13.2 Hz, 3H), 0.81 (s, 7H), 0.01 (s, 4H).

### Step 3—Methyl 2-(8-(benzo[d]thiazol-2-ylcarbamoyl)-3,4-dihydroisoquinolin-2 (1H)-yl)-5-(3-hydroxypropyl)thiazole-4-carboxylate

To a solution of methyl methyl 2-(8-(benzo[d]thiazol-2-ylcarbamoyl)-3,4-dihydroisoquinolin-2 (1H)-yl)-5-(3-((tert-butyldimethylsilyl)oxy)propyl)thiazole-4-carboxylate (12.0 g, 19.3 mmol) in DCM (5 mL) and MeOH (120 mL) was added to HCl/MeOH (0.2 M, 192 mL), then the mixture was stirred at 25° C., for 1 h. On completion, the reaction mixture was concentrated under reduced pressure to give a residue. The residue was purified by flash silica gel chromatography (Ethyl acetate/Petroleum ether=0/1 to 1/1) to give the title compound (3.50 g, 36% yield) as a brown solid. ¹H NMR (400 MHZ, DMSO-d₆) δ=12.90 (s, 1H), 8.04 (d, J=8.0 Hz, 1H), 7.79 (d, J=8.0 Hz, 1H), 7.67 (d, J=7.2 Hz, 1H), 7.51-7.43 (m, 2H), 7.42-7.33 (m, 2H), 5.75 (s, 1H), 4.82 (s, 2H), 3.75-3.72 (m, 2H), 3.71 (s, 3H), 3.42 (s, 2H), 3.03 (t, J=7.2 Hz, 4H), 1.77-1.61 (m, 3H).

### Methyl 2-(8-(benzo[d]thiazol-2-ylcarbamoyl)-3,4-dihydroisoquinolin-2 (1H)-yl)-5-(3-(4-iodophenoxy)propyl)thiazole-4-carboxylate (Intermediate BU)

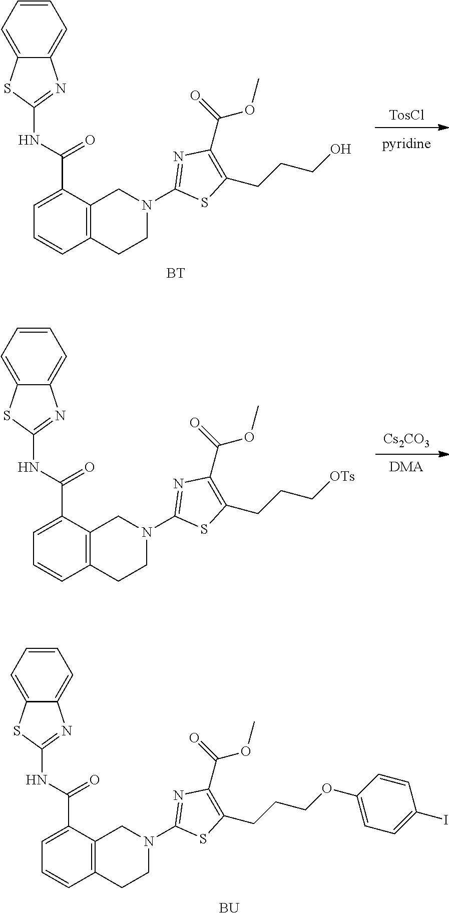

### Step 1—methyl 2-(8-(benzo[d]thiazol-2-ylcarbamoyl)-3,4-dihydroisoquinolin-2 (1H)-yl)-5-(3-(tosyloxy)propyl)thiazole-4-carboxylate

To a solution of methyl 2-(8-(benzo[d]thiazol-2-ylcarbamoyl)-3,4-dihydroisoquinolin-2 (1H)-yl)-5-(3-hydroxypropyl)thiazole-4-carboxylate (220 mg, 433 umol, Intermediate BT) in pyridine (3 mL) was added TosCl (315 mg, 1.65 mmol) at 0° C. The mixture was stirred at 25° C., for 2 h. On completion, the reaction mixture was diluted with water (10 mL), and extracted with EtOAc (15 mL×3). The combined organic layers were washed with brine (10 mL×3), dried over Na₂SO₄ and evaporated. The residue was purified by column chromatography (SiO₂, Petroleum ether/Ethyl acetate=2/1 to 1/1) to afford title compound (150 mg, 43% yield) as a yellow solid. LC-MS (ESI⁺) m/z 663.5 (M+H)⁺.

### Step 2—Methyl 2-(8-(benzo[d]thiazol-2-ylcarbamoyl)-3,4-dihydroisoquinolin-2 (1H)-yl)-5-(3-(4-iodophenoxy)propyl)thiazole-4-carboxylate

A solution of methyl 2-(8-(benzo[d]thiazol-2-ylcarbamoyl)-3,4-dihydroisoquinolin-2 (1H)-yl)-5-(3-(tosyloxy)propyl)thiazole-4-carboxylate (140 mg, 175 umol), 4-iodophenol (57.9 mg, 263 umol) and Cs₂CO₃ (85.7 mg, 263 umol) in DMA (2.8 mL) was stirred at 25° C., for 12 h. On completion, the reaction mixture was diluted with water (5 mL), and extracted with EtOAc (8 mL×3). The combined organic layers were washed with brine (2 mL×3), dried over Na₂SO₄ and evaporated. The crude product was purified by reversed-phase HPLC (CAN/0.1% FA=90%) to afford title compound (30.0 mg, 24% yield) was as a white solid. ¹H NMR (400 MHZ, CHLOROFORM-d) δ=7.88 (d, J=8.0 Hz, 1H), 7.68 (d, J=8.0 Hz, 1H), 7.59 (d, J=7.2 Hz, 1H), 7.55-7.50 (m, 2H), 7.45-7.41 (m, 1H), 7.37-7.32 (m, 3H), 6.66 (d, J=8.8 Hz, 2H), 4.90 (s, 2H), 3.96 (t, J=6.4 Hz, 2H), 3.89 (t, J=6.0 Hz, 2H), 3.79 (s, 3H), 3.27 (t, J=7.2 Hz, 2H), 3.06 (t, J=6.0 Hz, 2H), 2.16-2.08 (m, 2H).

### Methyl 2-(8-(benzo[d]thiazol-2-ylcarbamoyl)-3,4-dihydroisoquinolin-2 (1H)-yl)-5-(3-(4-(3-(piperazin-1-yl) prop-1-yn-1-yl)phenoxy)propyl)thiazole-4-carboxylate (Intermediate BV)

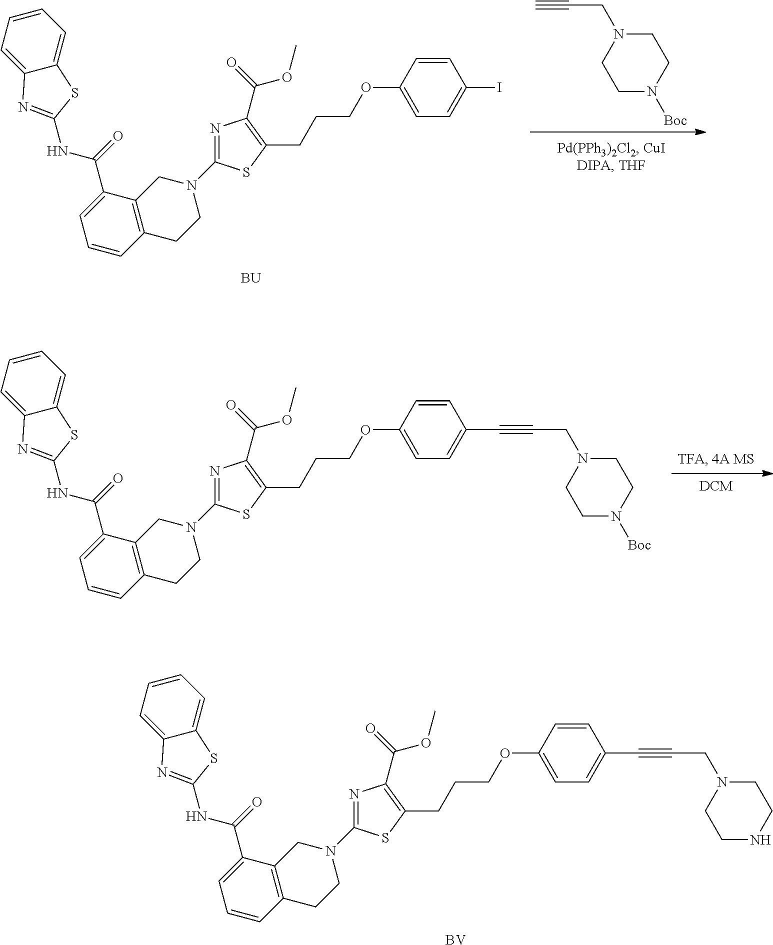

### Step 1—Methyl 2-(8-(benzo[d]thiazol-2-ylcarbamoyl)-3,4-dihydroisoquinolin-2 (1H)-yl)-5-(3-(4-(3-(4-(tert-butoxycarbonyl)piperazin-1-yl) prop-1-yn-1-yl)phenoxy)propyl)thiazole-4-carboxylate

To a solution of methyl 2-(8-(benzo[d]thiazol-2-ylcarbamoyl)-3,4-dihydroisoquinolin-2 (1H)-yl)-5-(3-(4-iodophenoxy)propyl)thiazole-4-carboxylate (180 mg, 253 umol, Intermediate BU), tert-butyl 4-(prop-2-yn-1-yl)piperazine-1-carboxylate (114 mg, 507 umol, CAS #199538-99-3) and DIPA (179 mg, 1.77 mmol) in THF (3.6 mL) was added Pd(PPh₃)₂Cl₂ (17.8 mg, 253 umol) and CuI (4.82 mg, 253 umol) under N₂. The mixture was stirred at 60° C., for 3 h. On completion, the reaction mixture was filtered and diluted with water (5 mL), and extracted with EtOAc (10 mL×3). The combined organic layers were washed with brine (5 mL×3), dried over Na₂SO₄ and evaporated. The residue was purified by column chromatography (SiO₂, Petroleum ether/Ethyl acetate=3/1 to 2/1) to afford the title compound (180 mg, 88% yield) as a yellow solid. LC-MS (ESI⁺) m/z 807.7 (M+H)⁺; ¹H NMR (400 MHZ, CDCl₃) δ=11.03-10.00 (m, 1H), 7.87 (d, J=7.6 Hz, 1H), 7.60 (t, J=7.2 Hz, 2H), 7.40-7.30 (m, 6H), 6.80 (d, J=8.4 Hz, 2H), 4.90 (s, 2H), 3.99 (t, J=6.0 Hz, 2H), 3.88 (t, J=6.0 Hz, 2H), 3.79 (s, 3H), 3.51 (s, 6H), 3.27 (t, J=7.6 Hz, 2H), 3.08-3.03 (m, 2H), 2.58 (s, 4H), 2.18-2.10 (m, 2H), 1.61 (s, 9H).

### Step 2—Methyl 2-(8-(benzo[d]thiazol-2-ylcarbamoyl)-3,4-dihydroisoquinolin-2 (1H)-yl)-5-(3-(4-(3-(piperazin-1-yl) prop-1-yn-1-yl)phenoxy)propyl)thiazole-4-carboxylate

To a solution of methyl 2-(8-(benzo[d]thiazol-2-ylcarbamoyl)-3,4-dihydroisoquinolin-2 (1H)-yl)-5-(3-(4-(3-(4-(tert-butoxycarbonyl)piperazin-1-yl) prop-1-yn-1-yl)phenoxy)propyl)thiazole-4-carboxylate (170 mg, 211 umol) and 4A molecular sieves (100 mg) in DCM (1.7 mL) was added TFA (524 mg, 4.59 mmol). The mixture was stirred at 25° C., for 1 h. On completion, the reaction mixture was poured into ice sat. NaHCO; (12 mL), and extracted with DCM (15 mL×3). The combined organic layers were washed with brine (5 mL×3), dried over Na₂SO₄ and evaporated. The residue was purified by prep-HPLC (column: Welch Xtimate C18 150*25 mm*5 um; mobile phase: [water (NH₃H₂O)-ACN]: B %: 47%-77%, 8 min) to afford the title compound (15.0 mg, 10% yield) as a white solid. LC-MS (ESI⁺) m/z 707.3 (M+H)⁺.

### Tert-butyl 3-(3-fluoro-2-hydroxyphenyl)propanoate (Intermediate BW)

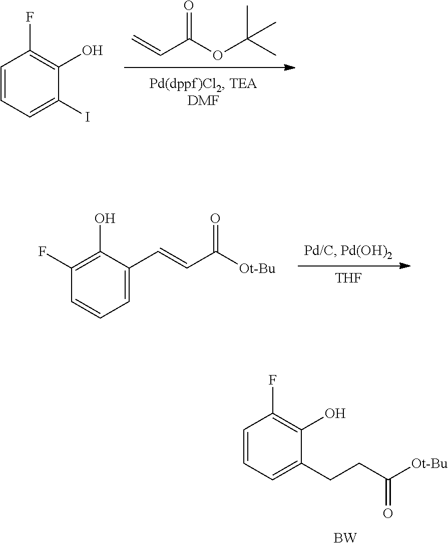

### Step 1—Tert-butyl (E)-3-(3-fluoro-2-hydroxy-phenyl)prop-2-enoate

A mixture of tert-butyl prop-2-enoate (200 mg, 1.56 mmol, 227 uL), 2-fluoro-6-iodo-phenol (371 mg, 1.56 mmol), Pd (dppf) Cl₂ (114 mg, 156 umol), and TEA (474 mg, 4.68 mmol, 651 uL) in DMF (1 mL), and then the mixture was stirred at 100° C., for 12 h at N₂ atmosphere. On completion, the reaction mixture was filtered and then concentrated under reduced pressure and quenched with H₂O (5 mL), and extracted with EtOAc (5 mL×3). The combined organic layers were washed with brine (5 mL×2), dried over Na₂SO₄, filtered and concentrated under reduced pressure to give a residue. The residue was purified by column chromatography (SiO₂, Petroleum ether/Ethyl acetate=5/1) to give the title compound (205 mg, 55% yield) as yellow solid. LC-MS (ESI⁺) m/z 238.1 (M+H)⁺.

### Step 2—Tert-butyl 3-(3-fluoro-2-hydroxyphenyl)propanoate

To a solution of tert-butyl 3-(3-fluoro-2-hydroxy-phenyl)prop-2-enoate (7 g, 29.4 mmol) in THF (100 mL) was added Pd/C (875 mg, 825 umol, 10 wt %), and Pd (OH) 2 (875 mg, 1.25 mmol, 20 wt %), then the mixture was stirred at 25° C., for 2 h under H₂ atmosphere. On completion, the reaction mixture was filtered and concentrated in vacuo and obtained the title compound (5 g, 71% yield) as yellow solid.

### Methyl 5-(3-(2-(3-(tert-butoxy)-3-oxopropyl)-6-fluorophenoxy)propyl)-2-chlorothiazole-4-carboxylate (Intermediate BX)

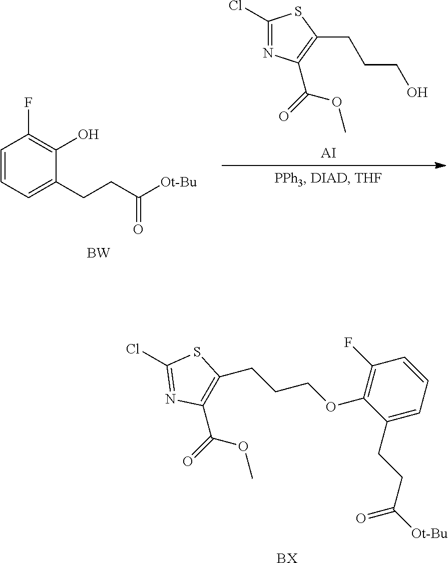

To a solution of tert-butyl 3-(3-fluoro-2-hydroxy-phenyl)propanoate (160 mg, 666 umol, Intermediate BW), methyl 2-chloro-5-(3-hydroxypropyl)thiazole-4-carboxylate (235 mg, 999 umol, Intermediate AH) and PPh; (262 mg, 999 umol) in THF (3.5 mL) was added DIAD (202 mg, 999 umol, 194 uL). The mixture was stirred at 0-25° C., for 12 h. On completion, the reaction mixture was quenched with H₂O (5 mL), extracted with EtOAc (5 mL×3). The combined organic layers were washed with brine (5 mL×2), dried over Na₂SO₄, filtered and concentrated under reduced pressure to give a residue. The residue was purified by column chromatography (SiO₂, Petroleum ether/Ethyl acetate=20/1 to 5/1) to give the title compound (200 mg) as brown gum. LC-MS (ESI⁺) m/z 457.11 (M+H)⁺.

### 3-(2-(3-(2-(8-(benzo[d]thiazol-2-ylcarbamoyl)-3,4-dihydroisoquinolin-2 (1H)-yl)-4-(methoxycarbonyl) thiazol-5-yl) propoxy)-3-fluorophenyl)propanoic acid (Intermediate BY)

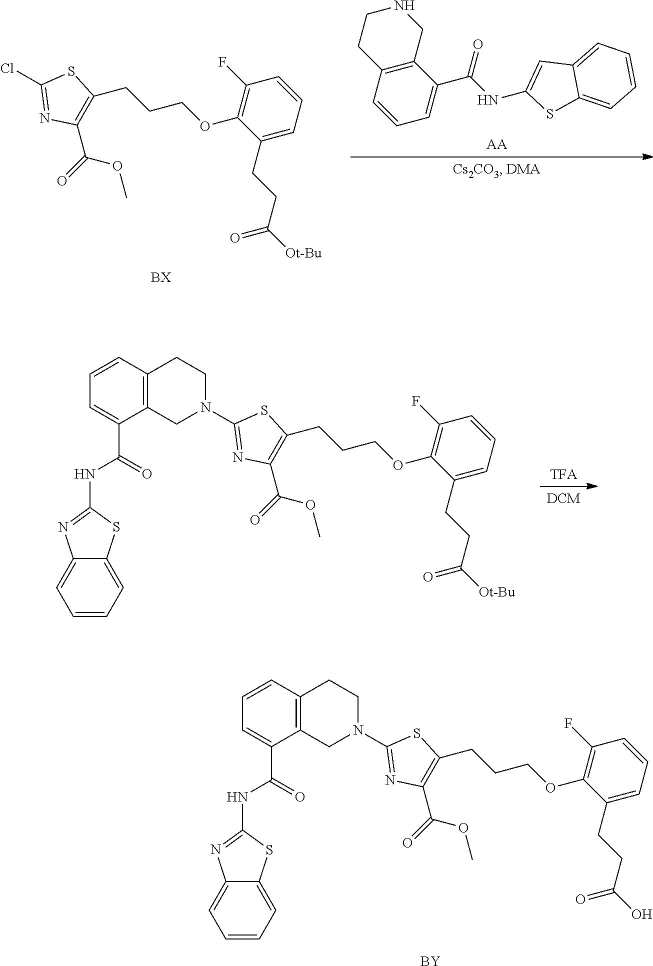

### Step 1—Methyl 2-(8-(benzo[d]thiazol-2-ylcarbamoyl)-3,4-dihydroisoquinolin-2 (1H)-yl)-5-(3-(2-(3-(tert-butoxy)-3-oxopropyl)-6-fluorophenoxy)propyl)thiazole-4-carboxylate

To a solution of methyl 5-(3-(2-(3-(tert-butoxy)-3-oxopropyl)-6-fluorophenoxy)propyl)-2-chlorothiazole-4-carboxylate (300 mg, 655 umol, Intermediate BX) and N-(1,3-benzothiazol-2-yl)-1,2,3,4-tetrahydroisoquinoline-8-carboxamide (203 mg, 655 umol, Intermediate AA) in DMA (3 mL) was added Cs₂CO₃ (640 mg, 1.97 mmol). The mixture was stirred at 100° C., for 12 h. On completion, the reaction mixture was quenched with THF (50 mL) and purified by flash silica gel chromatography (ISCOR: 4 g SepaFlash® Silica Flash Column, Eluent of 0˜50% THF/Petroleum ethergradient @ 100 mL/min) to give the title product (130 mg, 27% yield) as brown gum. LC-MS (ESI⁺) m/z 730.2 (M+H)⁺. ¹H NMR (400 MHz, DMSO-d₆) δ=12.90 (br s, 1H), 8.08-7.99 (m, 1H), 7.79 (br d, J=8.0 Hz, 1H), 7.74-7.63 (m, 1H), 7.50-7.44 (m, 2H), 7.43-7.25 (m, 3H), 7.10-7.03 (m, 1H), 7.02-6.94 (m, 2H), 4.83 (s, 2H), 4.02 (t, J=6.0 Hz, 2H), 3.71 (s, 2H), 3.24-3.18 (m, 2H), 3.04 (br t, J=6.0 Hz, 2H), 2.85-2.80 (m, 2H), 1.95 (s, 6H), 1.34-1.26 (m, 9H).

### Step 2—3-(2-(3-(2-(8-(benzo[d]thiazol-2-ylcarbamoyl)-3,4-dihydroisoquinolin-2 (1H)-yl)-4-(methoxycarbonyl) thiazol-5-yl) propoxy)-3-fluorophenyl)propanoic acid

To a solution of methyl 2-(8-(benzo[d]thiazol-2-ylcarbamoyl)-3,4-dihydroisoquinolin-2 (1H)-yl)-5-(3-(2-(3-(tert-butoxy)-3-oxopropyl)-6-fluorophenoxy)propyl)thiazole-4-carboxylate (130 mg, 178 umol) in DCM (1.5 mL) was added TFA (20.3 mg, 173 umol, 13.2 uL). The mixture was stirred at 20° C. for 2 h. On completion, the reaction mixture was quenched with NaHCO₃ (10 mL), and extracted with DCM (5 mL×3). The combined organic layers were washed with brine (5 mL×2), dried over Na₂SO₄, filtered and concentrated under reduced pressure to give the title compound (100 mg) as a yellow solid. LC-MS (ESI⁺) m z 674.1 (M+H)⁺. ¹H NMR (400 MHZ, DMSO-d₆) δ=8.07-7.86 (m, 1H), 7.78-7.54 (m, 2H), 7.48-7.21 (m, 4H), 7.11-6.67 (m, 3H), 4.81-4.72 (m, 1H), 4.04-3.83 (m, 2H), 3.72-3.57 (m, 4H), 3.17-3.09 (m, 2H), 3.03-2.66 (m, 7H), 2.01-1.83 (m, 3H), 1.25-1.03 (m, 2H).

### (2S,4R)-1-((S)-2-(7-aminoheptanamido)-3,3-dimethylbutanoyl)-4-hydroxy-N-((S)-1-(4-(4-methylthiazol-5-yl)phenyl)ethyl)pyrrolidine-2-carboxamide (Intermediate BZ)

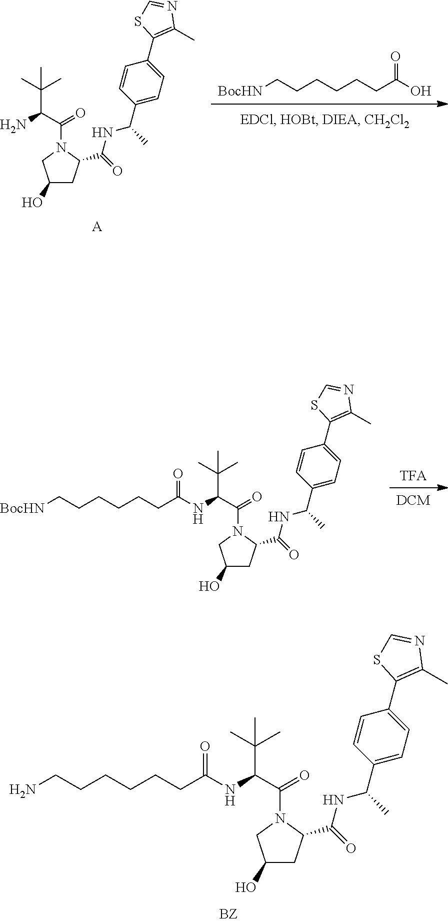

### Step 1—Tert-butyl (7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl) carbamate

To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (1.00 g, 2.25 mmol, Intermediate A) and 7-((tert-butoxycarbonyl)amino) heptanoic acid (827 mg, 3.37 mmol, CAS #60142-89-4) in CH₂Cl₂ (10 mL) was added EDCI (646 mg, 3.37 mmol), DIEA (1.45 g, 11.2 mmol) and HOBt (455 mg, 3.37 mmol), then the mixture was stirred at 25° C., for 2 h. On completion, the reaction mixture was quenched with sat. NH₄Cl (10 mL) and then extracted with EtOAc (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3:1 to DCM:MeOH=10:1) to give the title compound (1.30 g, 85% yield) as a colorless oil. LC-MS (ESI⁺) m/z 672.2 (M+H)⁺. ¹H NMR (400 MHZ, DMSO-d₆) δ=8.98 (s, 1H), 8.35 (d, J=8.0 Hz, 1H), 7.76 (d, J=9.2 Hz, 1H), 7.45-7.35 (m, 4H), 6.73 (s, 1H), 5.08 (d, J=3.6 Hz, 1H), 4.95-4.87 (m, 1H), 4.51 (d, J=9.2 Hz, 1H), 4.42 (t, J=8.0 Hz, 1H), 4.27 (s, 1H), 3.64-3.56 (m, 2H), 2.88 (q, J=6.4 Hz, 3H), 2.45 (s, 3H), 2.28-2.15 (m, 2H), 2.14-2.07 (m, 1H), 2.02 (d, J=7.2 Hz, 1H), 1.79 (ddd, J=4.4, 8.4, 12.8 Hz, 1H), 1.50-1.43 (m, 3H), 1.37 (s, 9H), 1.22 (d, J=6.0 Hz, 4H), 1.19-1.14 (m, 2H), 0.93 (s, 9H).

### Step 2—(2S,4R)-1-((S)-2-(7-aminoheptanamido)-3,3-dimethylbutanoyl)-4-hydroxy-N-((S)-1-(4-(4-methylthiazol-5-yl)phenyl)ethyl)pyrrolidine-2-carboxamide

To a solution of tert-butyl (7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl) carbamate (200 mg, 297 umol) in CH₂Cl₂ (3 mL) was added to TFA (1.54 g, 13.5 mmol), then the mixture was stirred at 25° C., for 12 h. On completion, the pH of the reaction mixture was adjusted to pH=8˜9 with NH₃·H₂O, and extracted with ethyl acetate (3 mL×3). The combined organic layers were washed with brine (3 mL×2), dried over Na₂SO₄, filtered and concentrated under reduced pressure to give the title compound (100 mg) as a colorless oil. ¹H NMR (400 MHz, DMSO-d₆) δ=8.98 (s, 1H), 8.40-8.34 (m, 1H), 7.79 (d, J=9.2 Hz, 1H), 7.48-7.40 (m, 4H), 7.39-7.36 (m, 2H), 5.03-4.83 (m, 2H), 4.56-4.47 (m, 1H), 4.41 (t, J=8.0 Hz, 1H), 4.28 (s, 1H), 3.60 (d, J=4.0 Hz, 2H), 2.72 (d, J=7.6 Hz, 2H), 2.45 (s, 3H), 2.26 (td, J=7.2, 14.4 Hz, 2H), 2.17-2.05 (m, 2H), 2.05-1.94 (m, 2H), 1.50-1.43 (m, 4H), 1.37 (d, J=6.8 Hz, 3H), 1.25 (dd, J=3.2, 8.4 Hz, 4H), 0.95-0.92 (m, 9H).

### 6-(8-(Benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid (Intermediate CA)

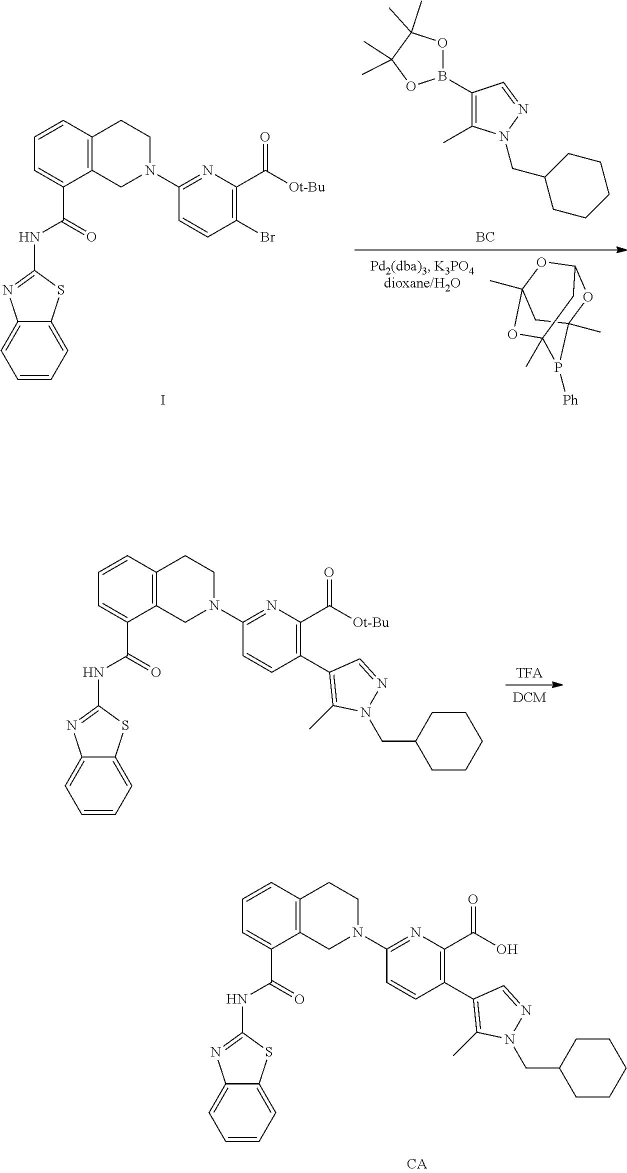

### Step 1—Tert-butyl 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinate

A mixture of tert-butyl 6-(8-(benzo[d]thiazol-2-ylcarbamoyl)-3,4-dihydroisoquinolin-2 (1H)-yl)-3-bromopicolinate (669 mg, 1.18 mmol, Intermediate I), 1-(cyclohexylmethyl)-5-methyl-4-(4,4,5,5-tetramethyl-1,3,2-dioxaborolan-2-yl)-1H-pyrazole (300 mg, 986 umol, Intermediate BC), (1S,3R,5R,7S)-1,3,5,7-tetramethyl-8-phenyl-2,4,6-trioxa-8-phosphatricyclo[3,3,1,1^{3,7}]decane (115 mg, 394 umol), K₃PO₄ (627 mg, 2.96 mmol) and Pd₂(dba); (90.3 mg, 98.6 umol) in dioxane (3 mL) and H₂O (3 mL) was degassed and purged with N₂ three times. Then the mixture was stirred at 100° C., for 2 h under N₂ atmosphere. On completion, the reaction mixture was quenched with sat. NH₄Cl (5 mL), and then extracted with EtOAc (5 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=5/1 to 1/1) to give the title compound (320 mg, 46% yield) as a yellow solid. LC-MS (ESI⁺) m/z 663.3 (M+H)⁺: ¹H NMR (400 MHZ, DMSO-d₆) δ=12.85 (s, 1H), 8.02 (d, J=8.0 Hz, 1H), 7.81-7.74 (m, 1H), 7.64-7.54 (m, 1H), 7.51-7.41 (m, 3H), 7.40-7.30 (m, 2H), 7.19 (s, 1H), 6.92 (d, J=8.8 Hz, 1H), 4.96 (s, 2H), 3.88-3.77 (m, 4H), 3.02 (t, J=6.0 Hz, 2H), 2.07 (s, 3H), 1.75 (ttd, J=3.6, 7.2, 14.0 Hz, 1H), 1.68-1.58 (m, 3H), 1.57-1.49 (m, 2H), 1.40 (d, J=9.2 Hz, 1H), 1.33 (s, 1H), 1.24 (d, J=5.2 Hz, 1H), 1.14 (s, 8H), 1.02-0.90 (m, 2H), 0.88-0.79 (m, 1H).

### Step 2—6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid

To a solution of tert-butyl 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinate (320 mg, 482 umol) in CH₂Cl₂ (3 mL) was added to TFA (1.64 g, 14.4 mmol), then the mixture was stirred at 25° C., for 12 h. On completion, the pH of reaction mixture was adjusted to pH=6˜7 with NH₃·H₂O and extracted with ethyl acetate (3 mL×3). The combined organic layers were washed with brine (3 mL×2), dried over Na₂SO₄, filtered and concentrated under reduced pressure to give a residue (300 mg, crude) as a yellow solid. Then 70.0 mg of the crude product was purified by prep-HPLC (column: Phenomenex luna C18 150*25 mm*10 um; mobile phase: [water (FA)-ACN]; B %: 62%-92%, 10 min) to give the title compound (12.5 mg, 18% yield) as a yellow solid. LC-MS (ESI⁺) m/z 607.5 (M+H)⁺: ¹H NMR (400 MHZ, DMSO-d₆) δ=8.03 (d, J=7.6 Hz, 1H), 7.79 (d, J=8.0 Hz, 1H), 7.61 (d, J=7.2 Hz, 1H), 7.50-7.42 (m, 3H), 7.38-7.33 (m, 2H), 7.31-7.27 (m, 1H), 6.91 (d, J=8.8 Hz, 1H), 4.94 (s, 2H), 3.88 (s, 2H), 3.83 (s, 2H), 3.01 (d, J=5.2 Hz, 2H), 2.11 (s, 3H), 1.81-1.75 (m, 1H), 1.67-1.61 (m, 2H), 1.52 (d, J=12.0 Hz, 2H), 1.26-1.20 (m, 1H), 1.19-1.11 (m, 3H), 1.01-0.92 (m, 2H).

### (2S,4R)—N-(2-(3-bromopropoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate CB)

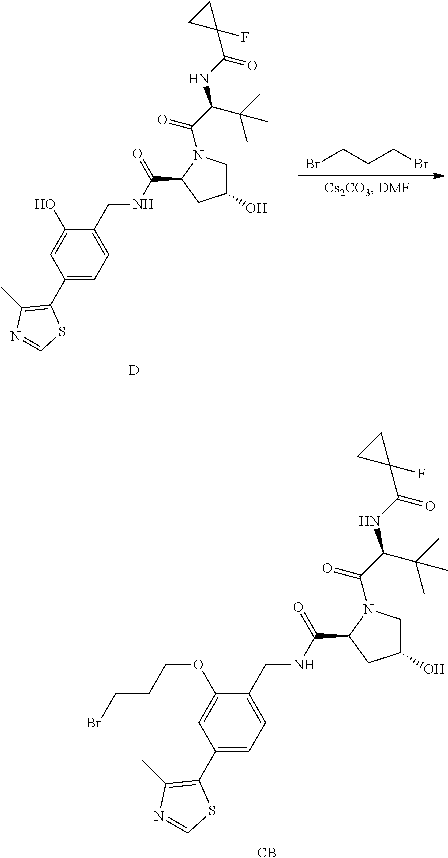

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (100 mg, 188 umol, Intermediate D), Cs₂CO₃ (122 mg, 376 umol) in DMF (6 mL) was added 1,3-dibromopropane (41.7 mg, 207 umol, 21.1 uL) at 60° C. The mixture was stirred at 60° C., for 2 h. On completion, the reaction mixture was poured into ice water (6 mL) and extracted with EtOAc (10 mL×3). The combined organic layers were washed with brine (10 mL×5), dried over sodium sulfate, filtered and concentrated under reduced pressure to give a residue. The residue was purified by flash silica gel chromatography (ISCO®; 4 g SepaFlash® Silica Flash Column, Eluent of 0˜100% Ethyl acetate/Petroleum ethergradient @60 mL/min) to give the title compound (120 mg, 47% yield) as a yellow oil. LC-MS (ESI⁺) m/z 653.17 (M+H)⁺.

### (2S,4R)-1-((S)-2-(4-aminobutanamido)-3,3-dimethylbutanoyl)-4-hydroxy-N-((S)-1-(4-(4-methylthiazol-5-yl)phenyl)ethyl)pyrrolidine-2-carboxamide (Intermediate CC)

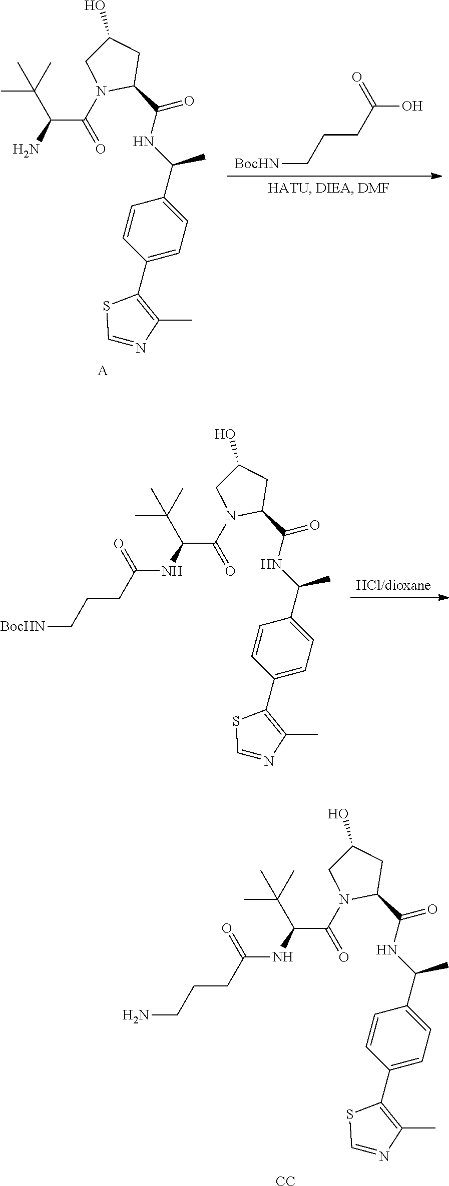

### Step 1—Tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl) carbamate

To a solution of 4-(tert-butoxycarbonylamino) butanoic acid (92.9 mg, 457 umol) and (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (220 mg, 457 umol, Intermediate A) in DMF (4 mL) was added DIEA (236 mg, 1.83 mmol) and HATU (217 mg, 571 umol) at 20° C., under nitrogen flow. Then the reaction was stirred at 20° C., for 10 h under nitrogen atmosphere. On completion, the reaction was poured into water (10 mL) and extracted with EtOAc (10 mL×2). The combined organic phase is washed with brine (3 mL×2), then dried over Na₂SO₄. Then the solution was filtered and the filtrate was concentrated to give a residue. The residue was purified by column chromatography on silica gel (eluted with petroleum ether: ethyl acetate=100:1 to 100:20) to give the title compound (230 mg, 62% yield) as a colorless oil. 1H NMR (400 MHz, CDCl₃) δ 8.71 (s, 1H), 7.46-7.33 (m, 4H), 6.98 (s, 1H), 5.09 (quin, J=7.2 Hz, 1H), 5.02 (s, 1H), 4.84-4.71 (m, 2H), 4.49 (br s, 2H), 4.19 (br d, J=11.0 Hz, 1H), 3.75 (br t, J=6.4 Hz, 1H), 3.58 (br d, J=10.4 Hz, 1H), 3.24-3.07 (m, 2H), 2.54 (s, 4H), 2.34-1.98 (m, 6H), 1.76 (br d, J=5.6 Hz, 2H), 1.46-1.44 (m, 9H), 1.08 (br s, 9H).

### Step 2—(2S,4R)-1-((S)-2-(4-aminobutanamido)-3,3-dimethylbutanoyl)-4-hydroxy-N-((S)-1-(4-(4-methylthiazol-5-yl)phenyl)ethyl)pyrrolidine-2-carboxamide

To a solution of tert-butyl (4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl) carbamate (230 mg, 365 umol) in HCl/dioxane (4 M, 91.3 uL) at 20° C., under nitrogen flow. Then the reaction was stirred at 20° C., for 1 h under nitrogen atmosphere. On completion, the reaction was concentrated to give the title compound (240 mg) as a yellow oil.

### (2S,4R)-4-hydroxy-1-((S)-2-(4-(3-(1-((4-iodo-5-methyl-1H-pyrazol-1-yl)methyl)cyclohexyl)propanamido)butanamido)-3,3-dimethylbutanoyl)-N-((S)-1-(4-(4-methylthiazol-5-yl)phenyl)ethyl)pyrrolidine-2-carboxamide (Intermediate CD)

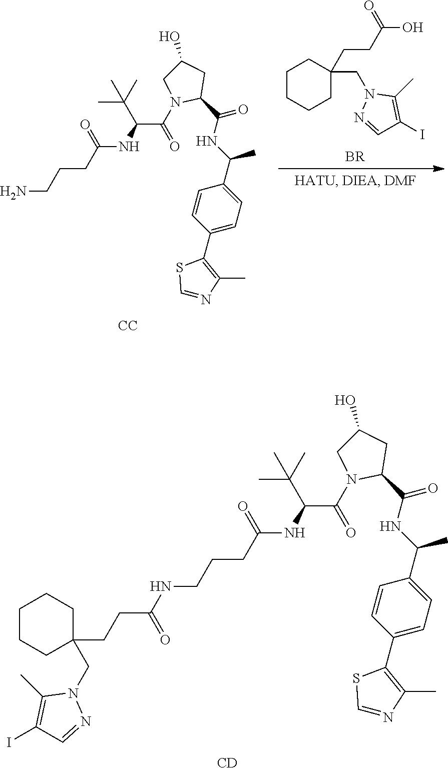

To a solution of 3-(1-((4-iodo-5-methyl-1H-pyrazol-1-yl)methyl)cyclohexyl)propanoic acid (180 mg, 478. Umol, Intermediate BR) and (2S,4R)-1-((S)-2-(4-aminobutanamido)-3,3-dimethylbutanoyl)-4-hydroxy-N-((S)-1-(4-(4-methylthiazol-5-yl)phenyl)ethyl)pyrrolidine-2-carboxamide (240 mg, 453 umol, Intermediate CC) in DMF (4 mL) was added DIEA (309 mg, 2.39 mmol) and HATU (200 mg, 526 umol) at 20° C., under nitrogen flow. Then the reaction was stirred at 20° C., for 1 h under nitrogen atmosphere. On completion, the reaction was poured into water (10 mL) and extracted with EtOAc (10 mL×2). The combined organic phase is washed with brine (3 mL×2), and dried over Na₂SO₄. The mixture was then filtered and the filtrate was concentrated to give a residue. The residue was purified by column chromatography on silica gel (eluted with petroleum ether:ethyl acetate=100:1 to 100:70) to give the title compound (150 mg, 32% yield) as a colorless oil. ¹H NMR (400 MHZ, CDCl₃) δ 7.54 (br d, J=7.9 Hz, 1H), 7.46-7.33 (m, 5H), 7.12 (br d, J=8.2 Hz, 1H), 7.00 (br t, J=5.6 Hz, 1H), 5.09 (br t, J=7.2 Hz, 1H), 4.77 (t, J=8.0 Hz, 1H), 4.57 (d, J=8.4 Hz, 1H), 4.48 (br s, 1H), 4.15 (br d, J=11.2 Hz, 1H), 3.91 (s, 2H), 3.58 (br dd, J=3.2, 11.2 Hz, 1H), 3.38-3.17 (m, 2H), 2.53 (s, 3H), 2.32 (s, 3H), 2.27-2.18 (m, 4H), 2.16-2.05 (m, 2H), 1.80 (tt, J=6.8, 13.8 Hz, 2H), 1.71 (br dd, J=5.6, 10.8 Hz, 2H), 1.47 (br d, J=6.8 Hz, 6H), 1.34 (br d, J=10.4 Hz, 3H), 1.29-1.14 (m, 4H), 1.07 (s, 9H).

(R)-2-((tert-butyldimethylsilyl)oxy)-1-(4-ethynylphenyl)ethanamine (Intermediate CE)

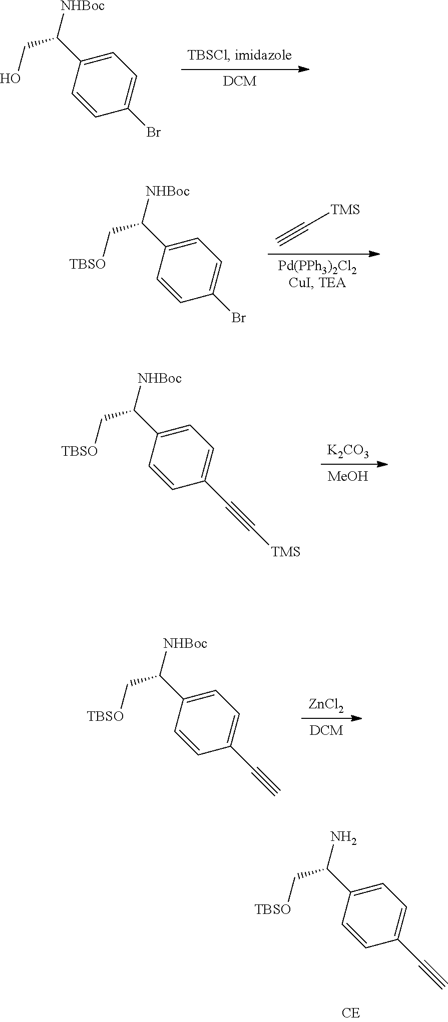

### Step 1—(R)-tert-butyl (1-(4-bromophenyl)-2-hydroxyethyl)carbamate

To a solution of tert-butyl N-[(1R)-1-(4-bromophenyl)-2-hydroxy-ethyl]carbamate (3.9 g, 12.33 mmol, CAS #3601-66-9) in DCM (40 mL) was added TBSCl (2.04 g, 13.57 mmol, 1.66 mL) and imidazole (1.68 g, 24.7 mmol). The mixture was stirred at 25° C., for 12 hr. On completion, the reaction mixture was quenched with water (40 mL), and extracted with EA (80 mL×3). The combined organic layers were washed with brine (80 mL), dried over anhydrous Na₂SO₄, filtered and concentrated under reduced pressure to afford the title compound (6.22 g).

### Step 2 (R)-tert-butyl (2-((tert-butyldimethylsilyl)oxy)-1-(4-((trimethylsilyl)ethynyl)phenyl)ethyl)carbamate

To a solution of tert-butyl N-[(1R)-1-(4-bromophenyl)-2-[tert-butyl(dimethyl)silyl]oxy-ethyl]carbamate (6.13 g, 14.2 mmol) in TEA (60 mL) was added ethynyl (trimethyl)silane (3.50 g, 35.6 mmol, 4.93 mL), dichloropalladium: triphenylphosphane (999.56 mg, 1.42 mmol) and CuI (271.22 mg, 1.42 mmol). The mixture was stirred at 65° C., for 12 hr. On completion, the reaction mixture was quenched with sat, aq NH₄Cl 40 mL, and then extracted with EA (100 mL×3). The combined organic layers were washed with brine (60 mL), dried over anhydrous Na₂SO₄, filtered and concentrated under reduced pressure to give a residue. The residue was purified by column chromatography (SiO₂, Petroleum ether/Ethyl acetate=100/1 to 50/1) to give the title compound (3.1 g, 44% yield as a yellow solid. LC-MS (ESI⁺) m/z 349.2 (M+H+H₂O)⁺.

### Step 3—(R)-tert-butyl (2-((tert-butyldimethylsilyl)oxy)-1-(4-ethynylphenyl)ethyl)carbamate

To a solution of tert-butyl N-[(IR)-2-[tert-butyl(dimethyl)silyl]oxy-1-[4-(2-trimethylsilylethynyl)phenyl]ethyl]carbamate (1 g, 2 mmol) in MeOH (10 mL) was added K₂CO₃ (370.40 mg, 2.68 mmol). Then the mixture was stirred at 25° C., for 1.5 hr. On completion, the mixture was filtered. The filtrate was washed with sat.aq NH₄Cl (5 mL), then with brine (10 mL), dried over anhydrous Na₂SO₄, filtered and concentrated under reduced pressure to give the title compound (950 mg, 24% yield) as a yellow solid. LC-MS (ESI⁺) m/z 320.2 (M+H-C4H₉)⁺.

### Step 4—(R)-2-((tert-butyldimethylsilyl)oxy)-1-(4-ethynylphenyl)ethanamine

To a solution of tert-butyl N-[(1R)-2-[tert-butyl(dimethyl)silyl]oxy-1-(4-ethynylphenyl)ethyl]carbamate (450 mg, 1.20 mmol) in DCM (3 mL) was added ZnCl₂ (326.61 mg, 2.40 mmol, 112.24 uL). The mixture was then stirred at 25° C., for 12 hr. On completion, the mixture was filtered. The filtrate was washed with H₂O (3 mL), then with brine (3 mL), dried over anhydrous Na₂SO₄, filtered and concentrated under reduced pressure to give a residue. The residue was purified by column chromatography (SiO₂, Petroleum ether:Ethyl acetate:Triethylamine=1:1:0.05) to give title compound (300 mg, 79% yield) as a yellow solid. LC-MS (ESI⁺) m/z 276.2 (M+H)⁺.

### (R)-7-(4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanoic acid (Intermediate CF)

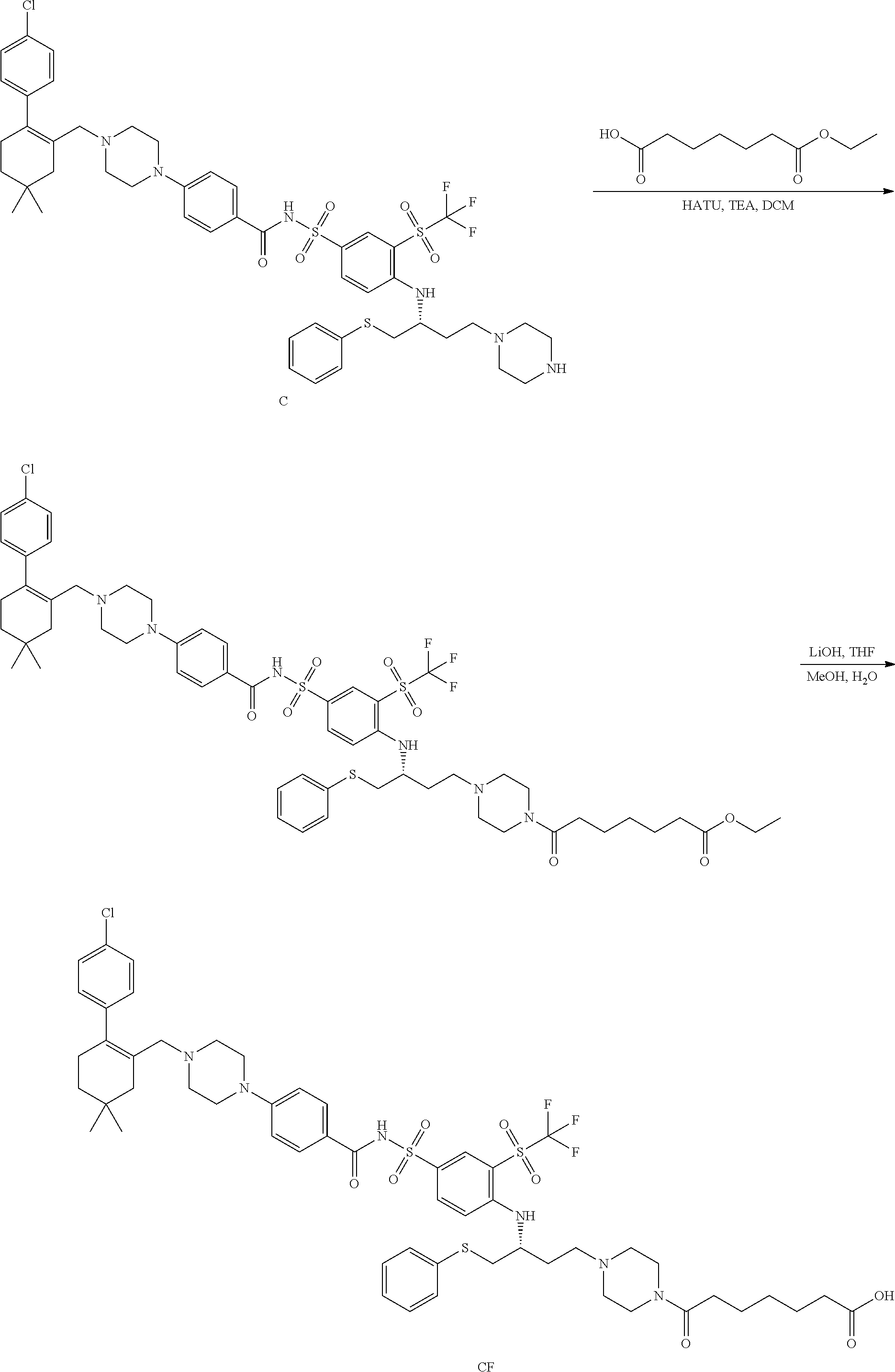

### Step 1—(R)-ethyl 7-(4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanoate

To a solution of 4-[4-[[2-(4-chlorophenyl)-5,5-dimethyl-cyclohexen-1-yl]methyl]piperazin-1-yl]-N-[4-[(1R)-1-(phenylsulfanylmethyl)-3-piperazin-1-yl-propyl]amino]-3-(trifluoromethylsulfonyl)phenyl]sulfonyl-benzamide (500 mg, 513 umol, Intermediate C), 7-ethoxy-7-oxo-heptanoic acid (96.7 mg, 513 umol) in DCM (5 mL) was added HATU (205 mg, 539 umol) and TEA (259 mg, 2.57 mmol, 357 uL). The mixture was stirred at 25° C., for 1 h. On completion, the mixture was washed with H₂O (5 mL×3), then with brine (3 mL×2), dried over anhydrous Na₂SO₄, filtered and concentrated under reduced pressure to give a residue. The residue was purified by column chromatography (SiO₂, Petroleum ether:Ethyl acetate=5:1 to Dichloromethane:Methanol=10:1) to give the title compound (540 mg, 92% yield) as a white solid. LC-MS (ESI⁺) m/z 1145.4 (M+Na)⁺.

### Step 2—(R)-7-(4-(3-((4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanoic acid

To a solution of ethyl 7-[4-[(3R)-3-[4-[4-[4-[[2-(4-chlorophenyl)-5,5-dimethyl-cyclohexen-1-yl]methyl]piperazin-1-yl]benzoyl]sulfamoyl]-2-(trifluoromethylsulfonyl) anilino]-4-phenylsulfanyl-butyl]piperazin-1-yl]-7-oxo-heptanoate (540 mg, 472 umol) in MeOH (2 mL), H₂O (2 mL), and THF (2 mL) was added LiOH·H₂O (99.1 mg, 2.36 mmol). The mixture was stirred at 25° C., for 2 h. On completion, the mixture was adjusted to acidity with HCl and concentrated in vacuo. The mixture was washed with H₂O (3 mL), extracted with EtOAc mL (2 mL×3), then with brine (3 mL×2), dried over anhydrous Na₂SO₄, filtered and concentrated under reduced pressure to give the title compound (370 mg) as a white solid. LC-MS (ESI⁺) m/z 1115.7 (M+H)⁺.

### (2S,4R)-1-((S)-2-(7-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxypyrrolidine-2-carboxylic acid (Intermediate CG)

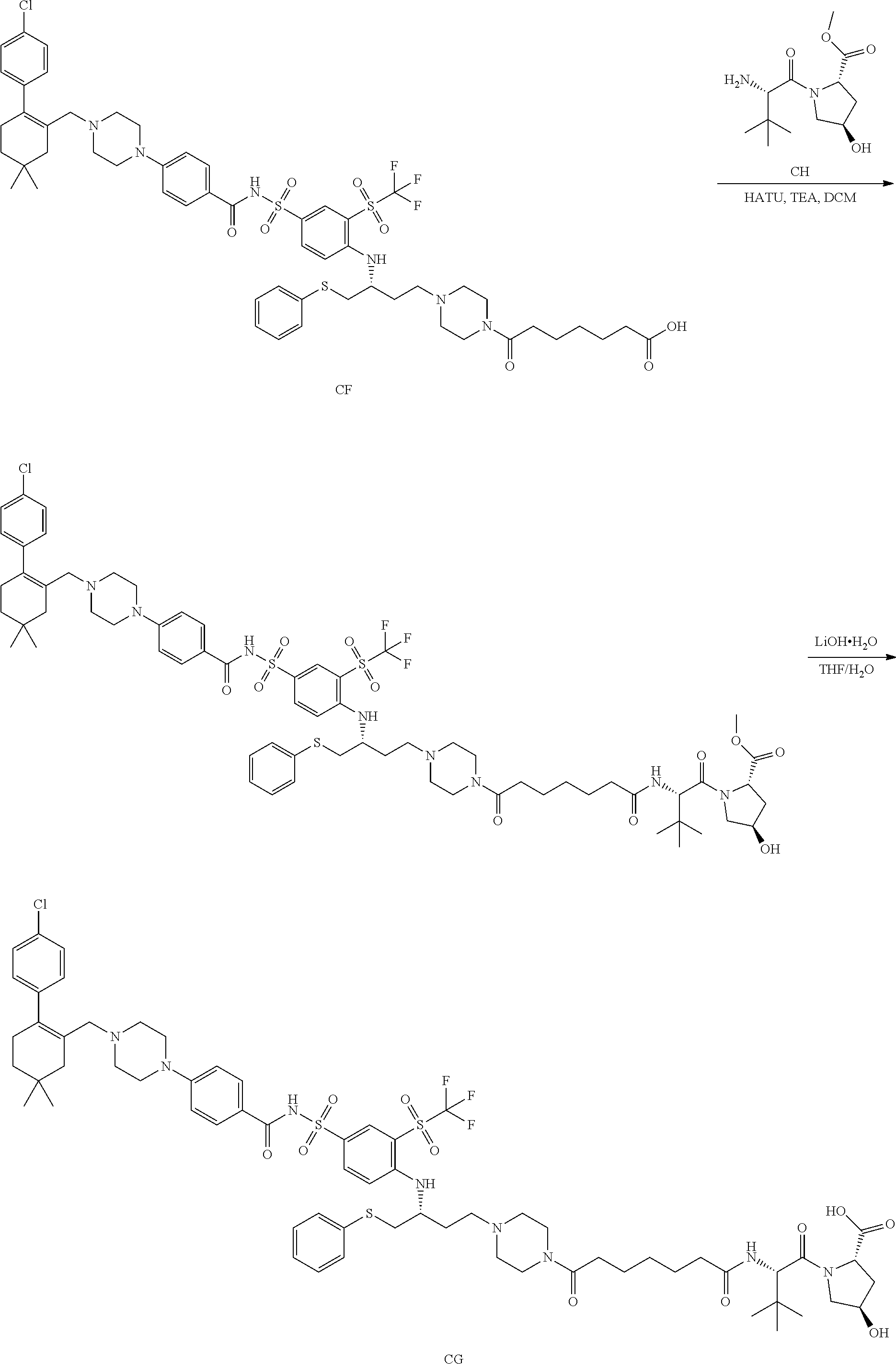

### Step 1—(2S,4R)-methyl 1-((S)-2-(7-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxypyrrolidine-2-carboxylate

To a solution of 7-[4-[(3R)-3-[4-[[4-[4-[2-(4-chlorophenyl)-5,5-dimethyl-cyclohexen-1-yl]methyl]piperazin-1-yl]benzoyl]sulfamoyl]-2-(trifluoromethylsulfonyl) anilino]-4-phenylsulfanyl-butyl]piperazin-1-yl]-7-oxo-heptanoic acid (370 mg, 331 umol, Intermediate CF) in DCM (5 mL) was added HATU (132 mg, 348 umol), methyl (2S,4R)-1-[(2S)-2-amino-3,3-dimethyl-butanoyl]-4-hydroxy-pyrrolidine-2-carboxylate (146 mg, 497 umol, HCl, Intermediate CH), and TEA (167 mg, 1.66 mmol, 230 uL), then the mixture was stirred at 25° C., for 1 h. On completion, the mixture was concentrated. The crude product was purified by reversed-phase HPLC (0.1% NH₃·H₂O) to give the title compound (360 mg, 80% yield) as a yellow solid. LC-MS (ESI⁺) m/z 1355.3 (M+H)⁺.

### Step 2—(2S,4R)-1-((S)-2-(7-(4-((R)-3-((4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxypyrrolidine-2-carboxylic acid

To a solution of methyl (2S,4R)-1-[(2S)-2-[[7-[4-[(3R)-3-[4-[4-[4-[[2-(4-chlorophenyl)-5,5-dimethyl-cyclohexen-1-yl]methyl]piperazin-1-yl]benzoyl]sulfamoyl]-2-(trifluoromethylsulfonyl) anilino]-4-phenylsulfanyl-butyl]piperazin-1-yl]-7-oxo-heptanoyl]amino]-3,3-dimethyl-butanoyl]-4-hydroxy-pyrrolidine-2-carboxylate (350 mg, 258 umol) in H₂O (1 mL), MeOH (1 mL), and THF (1 mL) was added LiOH·H₂O (21.6 mg, 516 umol), then the mixture was stirred at 25° C., for 2 h. On completion, the mixture was concentrated and quenched with water (5 mL), adjusted pH=4 with HCl (1 M), then extracted with EtOAc (5 mL×3). The combined organic layers was washed with brine (5 mL×3) and dried over Na₂SO₄ and concentrated in vacuo to give the title compound (300 mg) as a yellow solid. LC-MS (ESI⁺) m/z 1341.4 (M+1)⁺.

### (2S,4R)-methyl 1-((S)-2-amino-3,3-dimethylbutanoyl)-4-hydroxypyrrolidine-2-carboxylate (Intermediate CH)

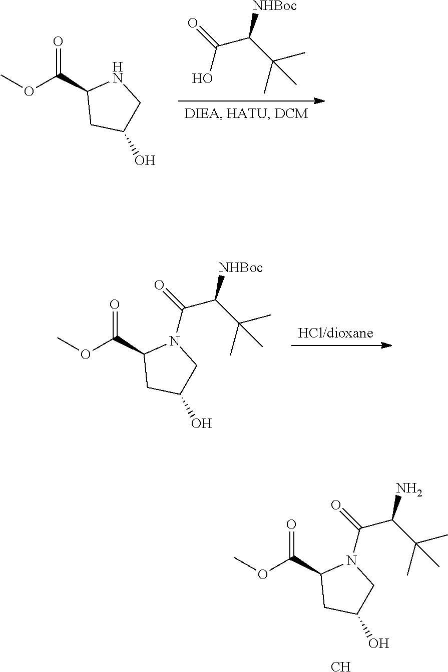

### Step 1—(2S,4R)-methyl-((S)-2-((tert-butoxycarbonyl)amino)-3,3-dimethylbutanoyl)-4-hydroxypyrrolidine-2-carboxylate

To a solution of (2S,4R)-methyl 4-hydroxypyrrolidine-2-carboxylate (10.0 g, 68.9 mmol, CAS #1499-56-5), (S)-2-((tert-butoxycarbonyl)amino)-3,3-dimethylbutanoic acid (15.9 g, 68.9 mmol, CAS #62965-35-9) in DCM (200 mL) was added HATU (39.3 g, 103 mmol) and DIEA (44.5 g, 344 mmol). The mixture was stirred at 25° C., for 12 h. On completion, the reaction mixture was partitioned between solvent DCM (100 mL×2) and H₂O (20 mL×2). The organic phase was separated, washed with brine (100 mL×2), dried over Na₂SO₄, filtered and concentrated under reduced pressure to give a residue. The residue was purified by column chromatography (SiO₂, Petroleum ether/Ethyl acetate=20/1 to 0/1) to give the title compound (20.0 g, 81% yield) as a brown oil. LC-MS (ESI⁺) m/z 359.0 (M+H)⁺.

### Step 2—(2S,4R)-methyl 1-((S)-2-amino-3,3-dimethylbutanoyl)-4-hydroxypyrrolidine-2-carboxylate

To a solution (2S,4R)-methyl of 1-((S)-2-((tert-butoxycarbonyl)amino)-3,3-dimethylbutanoyl)-4-hydroxypyrrolidine-2-carboxylate (20.0 g, 55.8 mmol) in DCM (40 mL) was added dropwise HCl/dioxane (4 M, 37.8 mL). The resulting mixture was stirred at 20° C., for 5 h. On completion, the reaction mixture was evaporated to give the title compound (17.0 g) as a white solid. LC-MS (ESI⁺) m z 259.1 (M+H)⁺.

### (2S,4R)-1-((S)-2-(1-fluorocyclopropanecarboxamido)-3,3-dimethylbutanoyl)-4-hydroxypyrrolidine-2-carboxylic acid (Intermediate CI)

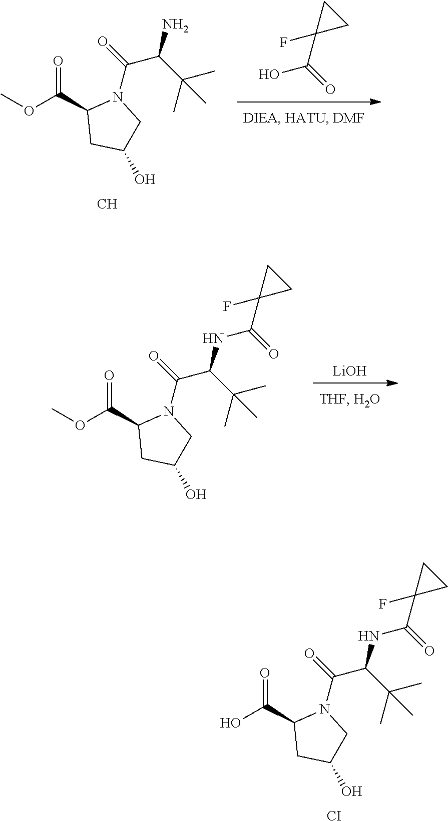

### Step 1—(2S,4R)-methyl 1-((S)-2-(1-fluorocyclopropanecarboxamido)-3,3-dimethylbutanoyl)-4-hydroxypyrrolidine-2-carboxylate

To a solution of (2S,4R)-methyl 1-((S)-2-amino-3,3-dimethylbutanoyl)-4-hydroxypyrrolidine-2-carboxylate (17.0 g, 57.7 mmol, HCl salt, Intermediate CH) and 1-fluorocyclopropanecarboxylic acid (6.00 g, 57.7 mmol) in DMF (170 mL) was added HATU (26.3 g, 69.2 mmol) and DIEA (74.5 g, 577 mmol). The mixture was stirred at 25° C., for 12 h. On completion, the reaction mixture was partitioned between solvent EtOAc (500 mL×4) and H₂O (250 mL). The organic phase was separated, washed with brine (100 mL×5), dried over Na₂SO₄, filtered and concentrated under reduced pressure to give a residue. The crude product was purified by column chromatography (SiO₂, Petroleum ether/Ethyl acetate=5/1 to 3/1) to give the title compound (11.0 g, 55% yield) as a brown oil. LC-MS (ESI⁺) m/z 345.2 (M+H)⁺.

### Step 2—(2S,4R)-1-((S)-2-(1-fluorocyclopropanecarboxamido)-3,3-dimethylbutanoyl)-4-hydroxypyrrolidine-2-carboxylic acid

To a solution of (2S,4R)-methyl 1-((S)-2-(1-fluorocyclopropanecarboxamido)-3,3-dimethylbutanoyl)-4-hydroxypyrrolidine-2-carboxylate (1.00 g, 2.90 mmol) in THF (10 mL) was added LiOH·H₂O (366 mg, 8.71 mmol) in H₂O (5 mL). The mixture was stirred at 25° C., for 2 h. On completion, the pH of the reaction mixture was adjusted to 4˜3 by addition 2 M HCl, then the mixture was extracted with EtOAc (10 mL×10). The organic phase was separated, dried over Na₂SO₄, filtered and concentrated under reduced pressure to give the title compound (720 mg) as a white oil.

### (2S,4R)-1-((S)-2-(1-fluorocyclopropanecarboxamido)-3,3-dimethylbutanoyl)-4-hydroxy-N-(2-hydroxybenzyl)pyrrolidine-2-carboxamide (Intermediate CJ)

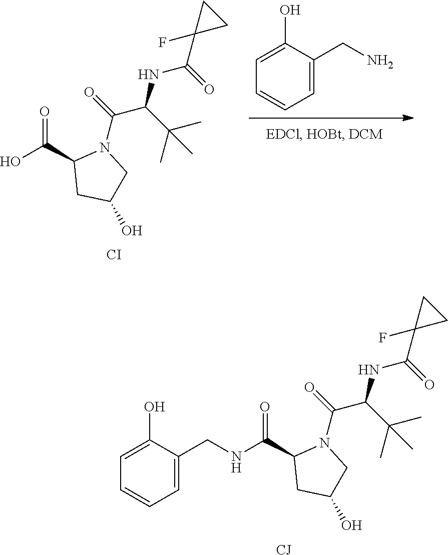

To a solution of (2S,4R)-1-((S)-2-(1-fluorocyclopropanecarboxamido)-3,3-dimethylbutanoyl)-4-hydroxypyrrolidine-2-carboxylic acid (300 mg, 900 umol, Intermediate CI) and 2-(aminomethyl)phenol (112 mg, 908 umol) in CH₂Cl₂ (3 mL) was added EDCI (192 mg, 999 umol) and HOBt (135 mg, 999 umol). The mixture was then stirred at 25° C., for 2 h. On completion, the reaction mixture was partitioned between solvent DCM (3 mL×3) and H₂O (6 mL×3). The organic phase was separated, dried over Na₂SO₄, filtered and concentrated under reduced pressure to give a residue. The crude product was purified by column chromatography (SiO₂, Petroleum ether: Dichloromethane=10/1 to 0/1) to give the title compound (190 mg, 48% yield) as a white solid. LC-MS (ESI⁺) m/z 436.2 (M+H)⁺.

### 2-(2-(((2S,4R)-1-((S)-2-(1-fluorocyclopropanecarboxamido)-3,3-dimethylbutanoyl)-4-hydroxypyrrolidine-2-carboxamido)methyl)phenoxy)acetic acid (Intermediate CK)

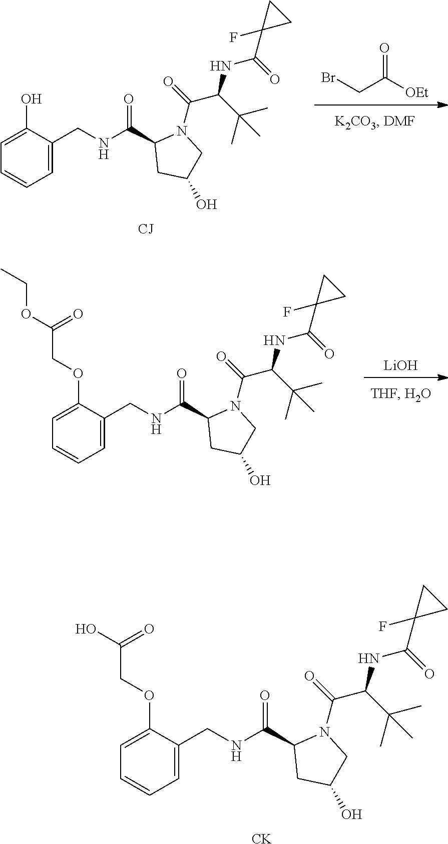

### Step 1—Ethyl 2-(2-(((2S,4R)-1-((S)-2-(1-fluorocyclopropanecarboxamido)-3,3-dimethylbutanoyl)-4-hydroxypyrrolidine-2-carboxamido)methyl)phenoxy)acetate

To a solution of (2S,4R)-1-((S)-2-(1-fluorocyclopropanecarboxamido)-3,3-dimethylbutanoyl)-4-hydroxy-N-(2-hydroxybenzyl)pyrrolidine-2-carboxamide (300 mg, 689 umol, Intermediate CJ) and ethyl 2-bromoacetate (173 mg, 1.03 mmol) in DMF (3 mL) was added K₂CO; (286 mg, 2.07 mmol) at 25° C. Then the reaction was stirred at 70° C., for 10 h under nitrogen atmosphere. On completion, the reaction mixture was partitioned between solvent EtOAc (6 mL×3) and H₂O (3 mL×3). The organic phase was separated, washed with brine (3 mL×3), dried over Na₂SO₄, filtered and concentrated under reduced pressure to give a residue. The crude product was purified by column chromatography (SiO₂, Dichloromethane:Methanol=60/1 to 50/1) to give the title compound (120 mg, 33% yield) as a white solid. LC-MS (ESI⁺) m/z 522.4 (M+H)⁺.

### Step 2—2-(2-(((2S,4R)-1-((S)-2-(1-fluorocyclopropanecarboxamido)-3,3-dimethylbutanoyl)-4-hydroxypyrrolidine-2-carboxamido)methyl)phenoxy)acetic acid

To a solution of ethyl 2-(2-(((2S,4R)-1-((S)-2-(1-fluorocyclopropanecarboxamido)-3,3-dimethylbutanoyl)-4-hydroxypyrrolidine-2-carboxamido)methyl)phenoxy)acetate (120 mg, 230 umol) in THF (1.2 mL) was added LiOH·H₂O (46.3 mg, 1.10 mmol) in H₂O (0.6 mL). The mixture was stirred at 20° C., for 2 h. On completion, the pH of the reaction mixture was adjusted to 4˜3 by addition of 2M HCl, then the mixture was extracted with EtOAc (2 ml×4). The organic phase was separated, dried over Na₂SO₄, filtered and concentrated under reduced pressure to give the title compound (80.0 mg) as a white oil. LC-MS (ESI⁺) m/z 494.2 (M+H)⁺.

### (2S,4R)—N-(4-fluoro-2-hydroxybenzyl)-1-((S)-2-(1-fluorocyclopropanecarboxamido)-3,3-dimethylbutanoyl)-4-hydroxypyrrolidine-2-carboxamide (Intermediate CL)

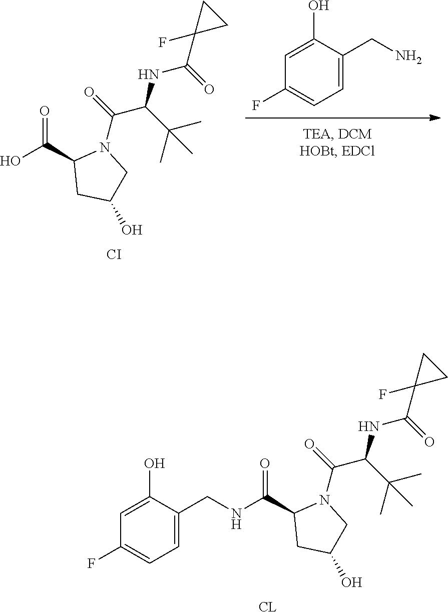

To a solution of (2S,4R)-1-[(2S)-2-[(1-fluorocyclopropanecarbonyl)amino]-3,3-dimethyl-butanoyl]-4-hydroxy-pyrrolidine-2-carboxylic acid (842 mg, 2.55 mmol, Intermediate CI), 2-(aminomethyl)-5-fluoro-phenol (360 mg, 2.55 mmol, and 2-(aminomethyl)-5-fluoro-phenol (360 mg, 2.55 mmol) in DCM (8 mL), was added HOBt (379 mg, 2.81 mmol), EDCI (537 mg, 2.81 mmol), and TEA (1.29 g, 12.8 mmol, 1.78 mL). The mixture was then stirred at 25° C., for 2 h. On completion, the mixture was washed with H₂O (10 mL×2), then with brine (10 mL×2), dried over anhydrous Na₂SO₄, filtered and concentrated under reduced pressure to give the title compound (160 mg, 14% yield) as a yellow solid. LC-MS (ESI⁺) m/z 454.3 (M+H)⁺.

### 2-(5-fluoro-2-(((2S,4R)-1-((S)-2-(1-fluorocyclopropanecarboxamido)-3,3-dimethylbutanoyl)-4-hydroxypyrrolidine-2-carboxamido)methyl)phenoxy)acetic acid (Intermediate CM)

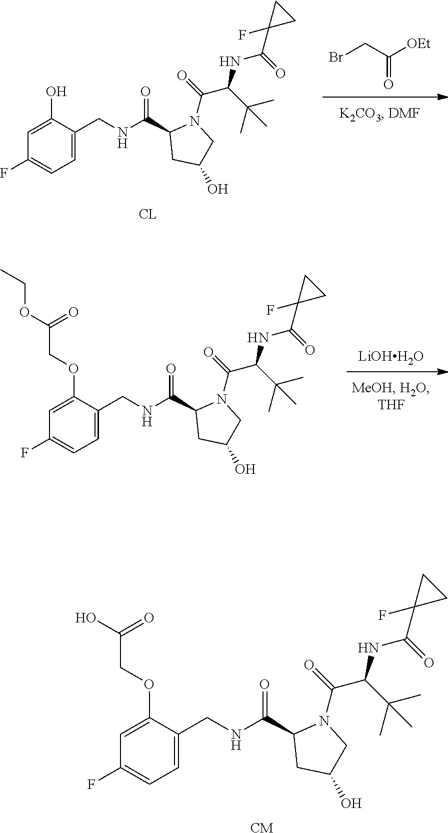

### Step 1—Ethyl 2-(5-fluoro-2-(((2S,4R)-1-((S)-2-(1-fluorocyclopropanecarboxamido)-3,3-dimethylbutanoyl)-4-hydroxypyrrolidine-2-carboxamido)methyl)phenoxy)acetate

To a solution of (2S,4R)-1-[(2S)-2-[(1-fluorocyclopropanecarbonyl)amino]-3,3-dimethyl-butanoyl]-N-[(4-fluoro-2-hydroxy-phenyl)methyl]-4-hydroxy-pyrrolidine-2-carboxamide (150 mg, 331 umol, Intermediate CL), ethyl 2-bromoacetate (82.8 mg, 496 umol, 54.9 uL) in DMF (2 mL) was added K₂CO₃ (137 mg, 992 umol). The mixture was stirred at 70° C., for 12 h. On completion, the mixture was filtered. The filtrate was extracted with EtOAc (2 mL×2), washed with brine (2 mL), dried over anhydrous Na₂SO₄, filtered and concentrated under reduced pressure to give a residue. The residue was purified by column chromatography (SiO₂, Petroleum ether:Ethyl acetate=5:1 to Dichloromethane:Methanol=10:1) to give the title compound (55 mg, 31% yield) as a white solid. LC-MS (ESI⁺) m/z 540.4 (M+H)⁺.

### Step 2 2-(5-Fluoro-2-(((2S,4R)-1-((S)-2-(1-fluorocyclopropanecarboxamido)-3,3-dimethylbutanoyl)-4-hydroxypyrrolidine-2-carboxamido)methyl)phenoxy)acetic acid

To a solution of ethyl 2-[5-fluoro-2-[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl phenoxy]acetate (55 mg, 102 umol) in MeOH (0.5 mL), H₂O (0.5 mL) and THF (0.5 mL) was added LiOH·H₂O (21.4 mg, 510 umol). The mixture was then stirred at 25° C., for 1 h. On completion, the mixture was adjusted pH to acidity and concentrated in vacuo. The mixture was washed with H₂O (3 mL), and extracted with EtOAc mL (2 mL×3). The combined organic phase was washed with brine (3 mL), dried over anhydrous Na₂SO₄, filtered and concentrated under reduced pressure to give the title compound (40 mg) as a white solid. LC-MS (ESI⁺) m/z 512.2 (M+H)⁺.

### Tert-butyl 3-bromo-6-((2-((tert-butoxycarbonyl)(methyl)amino)ethyl)amino)picolinate (Intermediate CN)

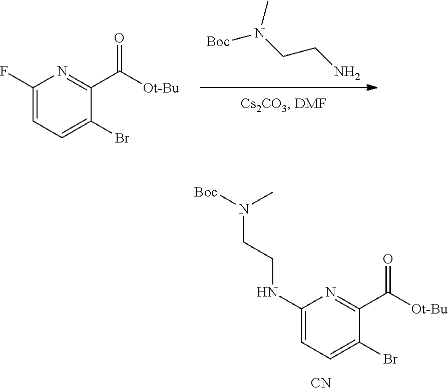

To a solution of tert-butyl (2-aminoethyl)(methyl)carbamate (3.00 g, 17.2 mmol, 3.08 mL, CAS #121492-06-6) and tert-butyl 3-bromo-6-fluoropicolinate (4.28 g, 15.5 mmol, CAS #1430753-76-6) in DMF (30 mL) was added Cs₂CO; (16.8 g, 51.7 mmol). The mixture was stirred at 60° C., for 17 h. On completion, the reaction mixture was quenched with NH₄Cl (30 mL) and extracted with EtOAC (50 mL×2). The combined organic layers were dried over Na₂SO₄ and evaporated. The residue was purified by silica gel column chromatography (Petroleum ether:Ethyl acetate=10:1 to 6:1) to give the title compound (3.53 g, 48% yield) as a white solid. ¹H NMR (400 MHZ, DMSO-d₆) δ 7.58 (br d, J=8.8 Hz, 1H), 7.17-6.95 (m, 1H), 6.50 (br d, J=8.8 Hz, 1H), 3.31 (br s, 2H), 2.83-2.75 (m, 3H), 2.52-2.50 (m, 2H), 1.52 (s, 9H), 1.34 (br s, 3H), 1.25 (br s, 6H).

### Tert-butyl 6-((2-((tert-butoxycarbonyl)(methyl)amino)ethyl)amino)-3-(1-(cyclohexylmethyl)-5-methyl-1H-pyrazol-4-yl)picolinate (Intermediate CO)

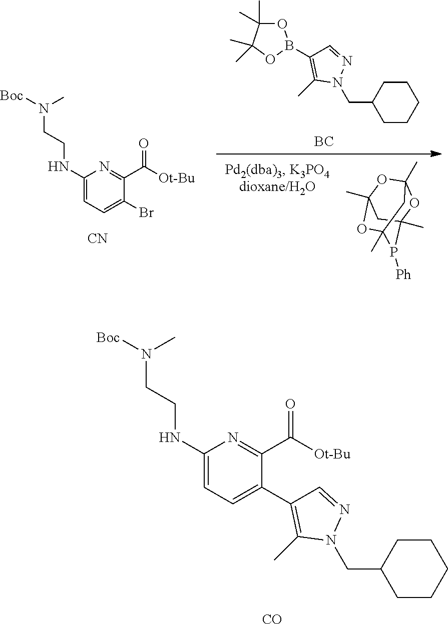

To a solution of tert-butyl 3-bromo-6-((2-((tert-butoxycarbonyl)(methyl)amino)ethyl)amino)picolinate (3.00 g, 6.97 mmol, Intermediate CN) and 1-(cyclohexylmethyl)-5-methyl-4-(4,4,5,5-tetramethyl-1,3,2-dioxaborolan-2-yl)-1H-pyrazole (2.12 g, 6.97 mmol, Intermediate BC) in dioxane (20 mL) and H₂O (20 mL) was added Pd₂(dba); (319 mg, 349 umol) and K₃PO₄ (4.44 g, 20.9 mmol) and (3R,5S)-1,3,5,7-tetramethyl-8-phenyl-2,4,6-trioxa-8-phosphaadamantane (408 mg, 1.39 mmol), then the mixture was stirred at 100° C., for 12 h under N₂. On completion, the reaction mixture was quenched with NH₄Cl (20 mL) and extracted with EtOAC (40 mL×3). The combined organic layers were washed with brine (15 mL×3), dried over Na₂SO₄ and evaporated. The residue was purified by silica gel column chromatography (Petroleum ether:Ethyl acetate=10:1 to 5:1) to give the title compound (3.53 g, 96% yield) as a white solid. ¹H NMR (400 MHZ, DMSO-d₆) δ 7.26 (d, J=8.8 Hz, 1H), 7.18 (s, 1H), 6.92-6.75 (m, 1H), 6.56 (d, J=8.8 Hz, 1H), 3.86 (d, J=7.2 Hz, 2H), 3.35 (br s, 3H), 2.87-2.78 (m, 4H), 2.08 (s, 3H), 1.77 (ddd, J=3.2, 7.2, 10.6 Hz, 1H), 1.69-1.52 (m, 6H), 1.38 (br d, J=19.6 Hz, 5H), 1.29-1.25 (m, 15H), 1.19-1.11 (m, 5H), 1.07 (s, 1H).

### N-(6-chloro-4-methylpyridazin-3-yl)-N-((2-(trimethylsilyl)ethoxy)methyl)benzo[d]thiazol-2-amine (Intermediate CP)

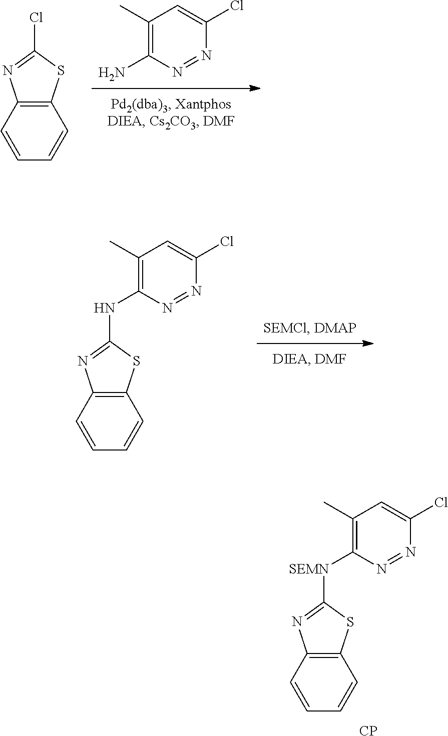

### Step 1—N-(6-chloro-4-methylpyridazin-3-yl)benzo[d]thiazol-2-amine

To a solution of 6-chloro-4-methyl-pyridazin-3-amine (5 g, 34.8 mmol, CAS #64068-00-4) and 2-chloro-1,3-benzothiazole (6.50 g, 38.3 mmol, CAS #615-20-3) in DMF (100 mL) was added DIEA (13.5 g, 104 mmol, 18.2 mL), Cs₂CO₃ (34.0 g, 104 mmol), Pd₂(dba); (1.59 g, 1.74 mmol) and XantPhos (2.02 g, 3.48 mmol). The mixture was then stirred at 75° C., for 4 h under N₂ atmosphere. On completion, the reaction mixture was poured into sat. NH₄Cl (100 mL) and extracted with EtOAc (200 mL×3). The combined organic layers were washed with brine (100 mL×8), dried over sodium sulfate, filtered and concentrated under reduced pressure to give a residue. The residue was purified by flash silica gel chromatography (ISCOR: 120 g SepaFlash® Silica Flash Column, Eluent of 0˜30% Ethyl acetate/Petroleum ethergradient (@ 80 mL/min) to give the title compound (4.9 g, 51% yield) as a green solid. ¹H NMR (400 MHZ, DMSO-d₆) δ 13.54-10.71 (m, 1H), 7.92-7.83 (m, 1H), 7.69-7.63 (m, 1H), 7.61-7.47 (m, 1H), 7.40 (br t, J=7.6 Hz, 1H), 7.23 (br t, J=7.6 Hz, 1H), 2.38 (s, 3H).

### Step 2 N-(6-chloro-4-methylpyridazin-3-yl)-N-((2-(trimethylsilyl)ethoxy)methyl)benzo[d]thiazol-2-amine

To a solution of N-(6-chloro-4-methyl-pyridazin-3-yl)-1,3-benzothiazol-2-amine (4.4 g, 15.90 mmol) in DMF (80 mL) was added DIEA (6.16 g, 47.70 mmol, 8.31 mL), and then 2-(chloromethoxy)ethyl-trimethyl-silane (7.95 g, 47.7 mmol, 8.44 mL) and DMAP (388 mg, 3.18 mmol) was added at 0° C. The mixture was stirred at 20° C., for 12 h. On completion, the reaction mixture was poured into ice water (100 mL) and extracted with EtOAc (200 mL×3). The combined organic layers were washed with brine (200 mL×5), dried over sodium sulfate, filtered and concentrated under reduced pressure to give a residue. The residue was purified by flash silica gel chromatography (ISCOR; 80 g SepaFlash® Silica Flash Column, Eluent of 0˜10% Ethyl acetate/Petroleum ethergradient @ 80 mL/min) to give the title compound (2.5 g, 32% yield) as a yellow solid. ¹H NMR (400 MHZ, DMSO-d₆) δ 7.83 (d, J=7.6 Hz, 1H), 7.67 (d, J=0.8 Hz, 1H), 7.52-7.42 (m, 2H), 7.30-7.24 (m, 1H), 5.86 (s, 2H), 3.70 (t, J=7.8 Hz, 2H), 2.37 (s, 3H), 0.92-0.86 (m, 2H), −0.13 (s, 9H).

### Ethyl 6-((6-(benzo[d]thiazol-2-ylamino)-5-methylpyridazin-3-yl)(2-(methylamino)ethyl)amino)-3-(1-(cyclohexylmethyl)-5-methyl-1H-pyrazol-4-yl)picolinate (Intermediate CQ)

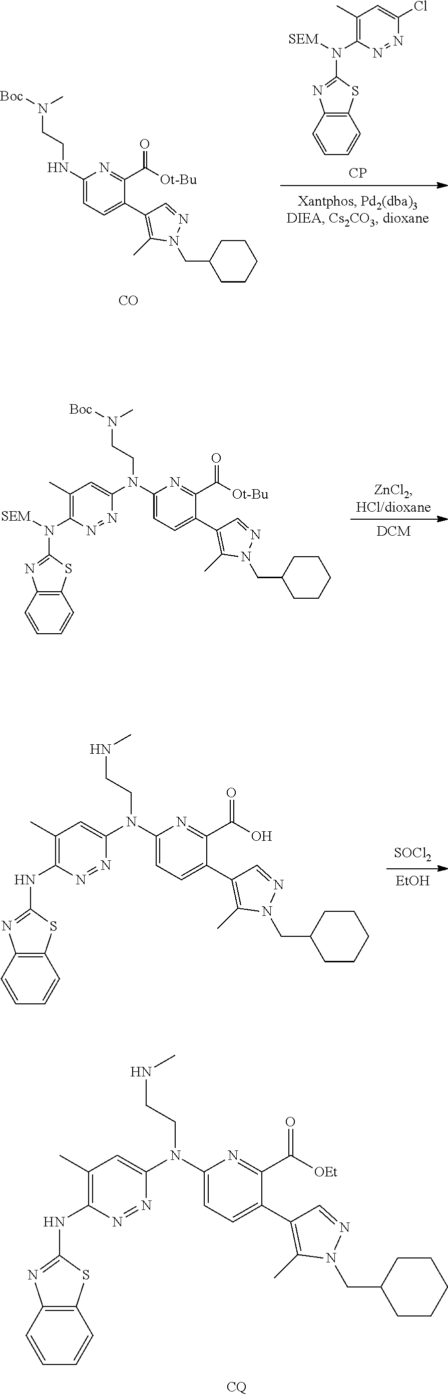

### Step 1—Tert-butyl 6-((6-(benzo[d]thiazol-2-yl((2-(trimethylsilyl)ethoxy)methyl)amino)-5-methylpyridazin-3-yl)(2-((tert-butoxycarbonyl)(methyl)amino)ethyl)amino)-3-(1-(cyclohexylmethyl)-5-methyl-1H-pyrazol-4-yl)picolinate

To a solution of methyl tert-butyl 6-((2-((tert-butoxycarbonyl)(methyl)amino)ethyl)amino)-3-(1-(cyclohexylmethyl)-5-methyl-1H-pyrazol-4-yl)picolinate (2.00 g, 3.79 mmol, Intermediate CO) and N-(6-chloro-4-methylpyridazin-3-yl)-N-((2-(trimethylsilyl)ethoxy)methyl)benzo[d]thiazol-2-amine (2.01 g, 4.93 mmol, Intermediate CP) in dioxane (25 mL) was added Xantphos (439 mg, 758 umol) and Pd₂(dba)₃ (347 mg, 379 umol), DIEA (1.47 g, 11.4 mmol, 1.98 mL), and Cs₂CO₃ (3.70 g, 11.4 mmol) under N₂. The mixture was stirred at 120° C., for 12 hr. On completion, the reaction mixture was quenched with NH₄Cl (20 mL) and extracted with EtOAC (40 mL×3). The combined organic layers were washed with brine (15 mL×3), dried over Na₂SO₄ and evaporated. The residue was purified by column chromatography (SiO₂, Petroleum ether/Ethyl acetate=10:1 to 5:1) to give the title compound (680 mg, 20% yield) as a white solid. ¹H NMR (400 MHZ, DMSO-d₆) δ 7.79 (d, J=7.6 Hz, 1H), 7.60-7.50 (m, 2H), 7.49-7.40 (m, 2H), 7.33-7.11 (m, 3H), 5.86 (s, 2H), 4.35 (br s, 2H), 3.90 (d, J=7.2 Hz, 2H), 3.72 (br t, J=8.0 Hz, 2H), 3.64-3.52 (m, 2H), 2.82-2.69 (m, 3H), 2.34 (s, 3H), 2.16 (s, 3H), 1.79 (br s, 1H), 1.70-1.53 (m, 5H), 1.34 (s, 8H), 1.29-1.19 (m, 12H), 1.18-1.13 (m, 3H), 0.92 (t, J=8.0 Hz, 2H), 0.86-0.76 (m, 2H), −0.11 (s, 9H).

### Step 2 6-((6-(benzo[d]thiazol-2-ylamino)-5-methylpyridazin-3-yl)(2-(methylamino)ethyl)amino)-3-(1-(cyclohexylmethyl)-5-methyl-1H-pyrazol-4-yl)picolinic acid

To a tert-butyl 6-((6-(benzo[d]thiazol-2-yl((2-(trimethylsilyl)ethoxy)methyl)amino)-5-methylpyridazin-3-yl)(2-((tert-butoxycarbonyl)(methyl)amino)ethyl)amino)-3-(1-(cyclohexylmethyl)-5-methyl-1H-pyrazol-4-yl)picolinate (680 mg, 757 umol) in DCM (7 mL) was added to ZnCl₂ (619 mg, 4.54 mmol, 213 uL), then the mixture was stirred at 25° C., for 36 h. Then, HCl/dioxane (6 mL) was added to the mixture which was then stirred at 25° C., for 2 h. On completion, the solution was concentrated in vacuo to afford the title compound (1.40 g) as an orange solid. LC-MS (ESI⁺) m/z 612.2 (M+H)⁺.

### Step 3—Ethyl 6-((6-(benzo[d]thiazol-2-ylamino)-5-methylpyridazin-3-yl)(2-(methylamino)ethyl)amino)-3-(1-(cyclohexylmethyl)-5-methyl-1H-pyrazol-4-yl)picolinate

To a stirred solution of 6-((6-(benzo[d]thiazol-2-ylamino)-5-methylpyridazin-3-yl)(2-(methylamino)ethyl)amino)-3-(1-(cyclohexylmethyl)-5-methyl-1H-pyrazol-4-yl)picolinic acid (1.30 g, 2.13 mmol) in EtOH (20 mL) at 0° C., was added SOCl₂ (758 mg, 6.38 mmol, 463 uL) dropwise. After addition, the mixture was stirred at 90° C., for 19 h. On completion, the solution was concentrated in vacuo. The crude product was purified by reversed-phase HPLC (water (0.05% FA)-ACN]: B %: 5%-53%, 50 min) to give the title compound (100 mg, 7% yield) as an orange solid. LC-MS (ESI⁺) m/z 640.2 (M+H)⁺.

### 6-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)hexanoic acid (Intermediate CR)

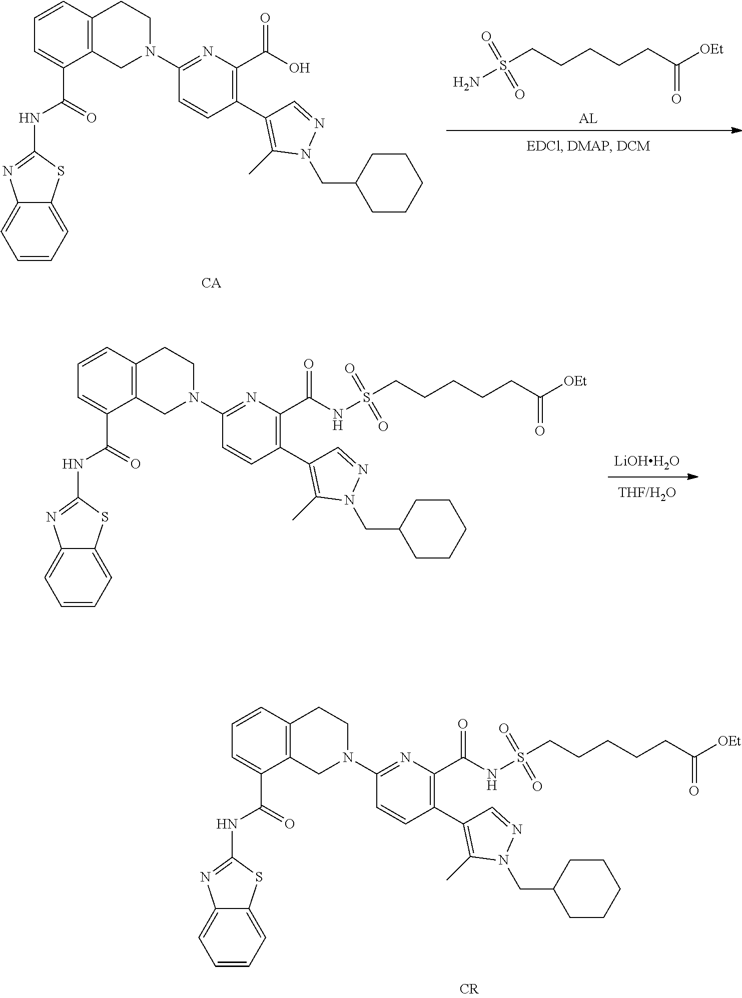 [SMILES: Cc1c(-c2ccc(N3CCc4cccc(C(=O)Nc5nc6ccccc6s5)c4C3)nc2C(=O)O)cnn1CC1CCCCC1 ; CCOC(=O)CCCCCS(N)(=O)=O ; "AL" ; CCOC(=O)CCCCCS(=O)(=O)NC(=O)c1nc(N2CCc3cccc(C(=O)Nc4nc5ccccc5s4)c3C2)ccc1-c1cnn(CC2CCCCC2)c1C ; "LiOH·H2O" ; CCOC(=O)CCCCCS(=O)(=O)NC(=O)c1nc(N2CCc3cccc(C(=O)Nc4nc5ccccc5s4)c3C2)ccc1-c1cnn(CC2CCCCC2)c1C]

### Step 1—Ethyl 6-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)hexanoate

To a solution of ethyl 6-sulfamoylhexanoate (214 mg, 956 umol, Intermediate AL), 6-(8-(benzo[d]thiazol-2-ylcarbamoyl)-3,4-dihydroisoquinolin-2 (1H)-yl)-3-(1-(cyclohexylmethyl)-5-methyl-IH-pyrazol-4-yl)picolinic acid (290 mg, 478 umol, Intermediate CA) in DCM (4 mL) was added EDCI (458 mg, 2.39 mmol), and DMAP (292 mg, 2.39 mmol). The mixture was then stirred at 25° C., for 17 h. On completion, the solution was concentrated in vacuo. The mixture was further purification by pre-HPLC (column: Phenomenex C18 250*50 mm*10 um; mobile phase: [water (NH₄HCO₃)-ACN]; B %: 35%-65%, 8 min) to give the title compound (255 mg, 66% yield) as an orange solid. LC-MS (ESI⁺) m/z 812.3 (M+H)+.

### Step 2—6-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)hexanoic acid

To a solution of ethyl 6-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]hexanoate (245 mg, 302 umol) in THF (4 mL) and H₂O (1 mL) was added LiOH·H₂O (127 mg, 3.02 mmol), then the mixture was stirred at 25° C., for 13 h. On completion, the reaction mixture was filtered and the pH of the aqueous phase was adjusted to 4˜5 by addition of 2M HCl, then extracted with EtOAC (5 mL×2). The combined organic layers were dried over Na₂SO₄ and evaporated to afford the title compound (155 mg) as an orange solid. LC-MS (ESI⁺) m/z 784.2 (M+H)⁺.

### 6-(8-(benzo[d]thiazol-2-ylcarbamoyl)-3,4-dihydroisoquinolin-2 (1H)-yl)-3-(1,5-dimethyl-1H-pyrazol-4-yl)picolinic acid (Intermediate CS)

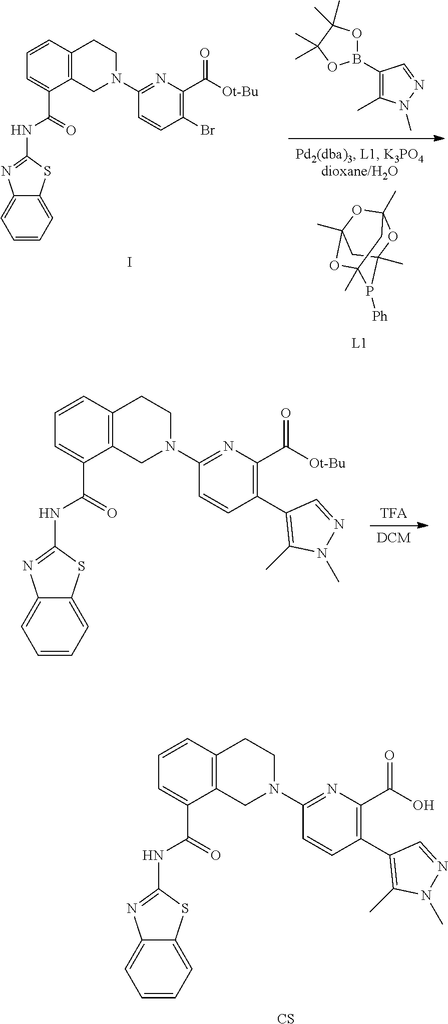

### Step 1—tert-butyl 6-(8-(benzo[d]thiazol-2-ylcarbamoyl)-3,4-dihydroisoquinolin-2 (1H)-yl)-3-(1,5-dimethyl-1H-pyrazol-4-yl)picolinate

To a solution of tert-butyl 6-(8-(benzo[d]thiazol-2-ylcarbamoyl)-3,4-dihydroisoquinolin-2 (1H)-yl)-3-bromopicolinate (500 mg, 884 umol, Intermediate I) and 1,5-dimethyl-4-(4,4,5,5-tetramethyl-1,3,2-dioxaborolan-2-yl)-1H-pyrazole (295 mg, 1.33 mmol) in dioxane (5 mL) and H₂O (5 mL) was added Pd₂(dba); (81.0 mg, 88.4 umol), (1S,3R,5R,7S)-1,3,5,7-tetramethyl-8-phenyl-2,4,6-trioxa-8-phosphatricyclo[3,3,1,1^{3,7}]decane (103 mg, 354 umol) and K₃PO₄ (563 mg, 2.65 mmol). Then the mixture was stirred at 100° C., for 12 h under N₂. On completion, the reaction mixture was poured into water (10 mL), and extracted with EtOAc (10 mL×3). The combined organic layer was washed with brine (10 mL×3), dried over Na₂SO₄, filtered and concentrated under reduced pressure to give a residue. The residue was purified by column chromatography (SiO₂, Petroleum ether/Ethyl acetate=20/1-5/1) to give the title compound (400 mg, 76% yield) as a white solid. ¹H NMR (400 MHZ, CDCl₃) δ=10.11-9.97 (m, 1H), 7.86 (d, J=7.8 Hz, 1H), 7.65-7.60 (m, 1H), 7.58-7.53 (m, 1H), 7.44-7.28 (m, 6H), 6.85 (d, J=8.8 Hz, IH), 5.03 (s, 2H), 4.05 (t, J=6.0 Hz, 2H), 3.81 (s, 3H), 3.06 (t, J=6.0 Hz, 2H), 2.14 (s, 3H), 0.99-0.89 (m, 9H).

### Step 2—6-(8-(benzo[d]thiazol-2-ylcarbamoyl)-3,4-dihydroisoquinolin-2 (1H)-yl)-3-(1,5-dimethyl-1H-pyrazol-4-yl)picolinic acid

To a solution of tert-butyl 6-(8-(benzo[d]thiazol-2-ylcarbamoyl)-3,4-dihydroisoquinolin-2 (1H)-yl)-3-(1,5-dimethyl-1H-pyrazol-4-yl)picolinate (200 mg, 344 umol) in DCM (2 mL) was added TFA (0.5 mL), then the mixture was stirred at 25° C., for 3 h. On completion, the reaction mixture was concentrated. The residue was purified by prep-HPLC (column: Phenomenex luna C18 150*25 mm*10 um; mobile phase: [water (FA)-ACN]; B %: 37%-67%, 10 min) to give the title compound (20 mg, 11% yield) as a yellow solid. ¹H NMR (400 MHZ, DMSO-d₆) δ=13.08-12.50 (m, 1H), 8.04 (d, J=7.4 Hz, 1H), 7.80 (d, J=8.0 Hz, 1H), 7.62 (d, J=7.2 Hz, 1H), 7.52-7.42 (m, 3H), 7.40-7.34 (m, 2H), 7.26 (s, 1H), 6.94 (d, J=9.0 Hz, 1H), 4.95 (s, 2H), 3.89 (br t, J=5.6 Hz, 2H), 3.73 (s, 3H), 3.01 (br t, J=5.6 Hz, 2H), 2.14 (s, 3H).

### 6-(N-(6-(8-(benzo[d]thiazol-2-ylcarbamoyl)-3,4-dihydroisoquinolin-2 (1H)-yl)-3-(1,5-dimethyl-1H-pyrazol-4-yl)picolinoyl)sulfamoyl)hexanoic acid (Intermediate CT)

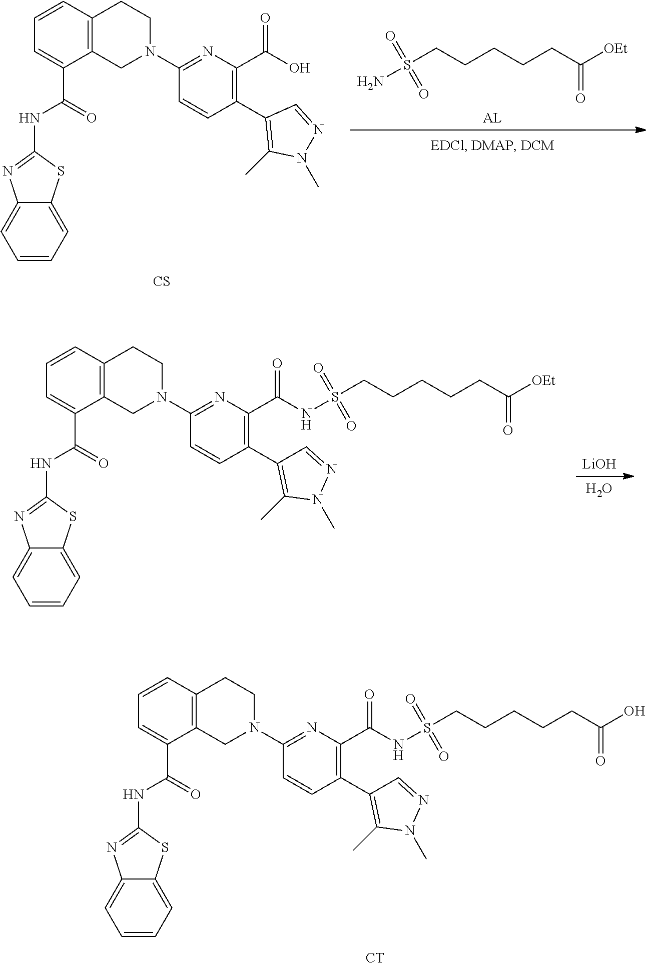

### Step 1—Ethyl 6-(N-(6-(8-(benzo[d]thiazol-2-ylcarbamoyl)-3,4-dihydroisoquinolin-2 (1H)-yl)-3-(1,5-dimethyl-1H-pyrazol-4-yl)picolinoyl)sulfamoyl)hexanoate

To a solution of 6-(8-(benzo[d]thiazol-2-ylcarbamoyl)-3,4-dihydroisoquinolin-2 (1H)-yl)-3-(1,5-dimethyl-1H-pyrazol-4-yl)picolinic acid (150 mg, 286 umol, Intermediate CS), ethyl 6-sulfamoylhexanoate (63.9 mg, 286 umol, Intermediate AL) in DCM (1.5 mL) was added EDCI (274 mg, 1.43 mmol) and DMAP (175 mg, 1.43 mmol), then the mixture was stirred at 25° C., for 12 h. On completion, the mixture was concentrated under reduced pressure to give a residue. The residue was purified by column chromatography (SiO₂, Dichloromethane:Methanol=20/1-10/1) to give the title compound (100 mg, 23.96% yield) as a white oil. LC-MS (ESI⁺) m/z 730.5 (M+H)⁺.

### Step 2—6-(N-(6-(8-(benzo[d]thiazol-2-ylcarbamoyl)-3,4-dihydroisoquinolin-2 (1H)-yl)-3-(1,5-dimethyl-1H-pyrazol-4-yl)picolinoyl)sulfamoyl)hexanoic acid

To a solution of ethyl 6-(N-(6-(8-(benzo[d]thiazol-2-ylcarbamoyl)-3,4-dihydroisoquinolin-2 (1H)-yl)-3-(1,5-dimethyl-1H-pyrazol-4-yl)picolinoyl)sulfamoyl)hexanoate (80 mg, 50 umol) in THF (1 mL) and H₂O (0.2 mL) was added LiOH (2.63 mg, 110 umol), then the mixture was stirred at 40° C., at 16 h. On completion, the reaction mixture was quenched with H₂O (2 mL) and extracted with EtOAc (2 mL×3). The aqueous phase was adjusted to pH=3˜4 by addition of HCl, then the mixture was extracted with EtOAC (2 mL×3). The combined organic layer was washed with brine (2 mL×3), dried over Na₂SO₄ and evaporated to give the title compound (30 mg) as a yellow solid. LC-MS (ESI⁺) m/z 702.5 (M+H)⁺.

### 6-[[4-[4-[[2-(4-chlorophenyl)-5,5-dimethyl-cyclohexen-1-yl]methyl]piperazin-1-yl]benzoyl]sulfamoyl]hexanoic acid (Intermediate CU)

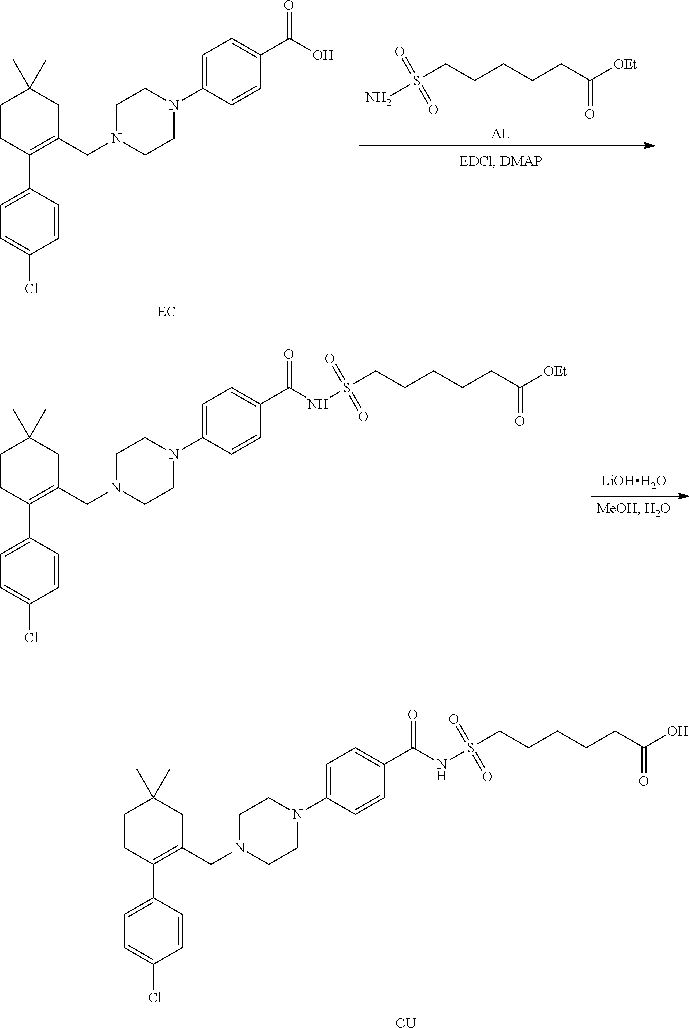

### Step 1—Ethyl 6-[[4-[4-[[2-(4-chlorophenyl)-5,5-dimethyl-cyclohexen-1-yl]methyl]piperazin-1-yl]benzoyl]sulfamoyl]hexanoate

To a solution of 4-[4-[[2-(4-chlorophenyl)-5,5-dimethyl-cyclohexen-1-yl]methyl]piperazin-1-yl]benzoic acid (200 mg, 455 umol, Intermediate EC) in DCM (8 mL) was added EDCI (174 mg, 911 umol) and DMAP (111 mg, 911 umol). Then ethyl 6-sulfamoylhexanoate (203 mg, 911 umol, Intermediate AL) was added to the reaction and stirred at 25° C., for 4 hrs. On completion, the reaction mixture was diluted with water (10 ml) and extracted with DCM (20 mL×2). The combined organic layers were washed with saturated brine and dried over Na₂SO₄, filtered and concentrated in vacuo to give a crude product. The crude product was purified by prep-HPLC (column: Phenomenex luna C18 150*25 mm*10 um: mobile phase: [water (FA)-ACN]: B %: 25%-55%, 10.5 min) to give the title compound (110 mg, 37% yield) as white solid. ¹H NMR (400 MHZ, DMSO-d₆) δ=8.13 (s, 1H), 7.83 (d, J=8.8 Hz, 2H), 7.45-7.38 (m, 2H), 7.16 (d, J=8.4 Hz, 2H), 6.97 (d, J=8.8 Hz, 2H), 4.06-3.98 (m, 3H), 3.94-3.80 (m, 1H), 3.66-3.56 (m, 1H), 3.52-3.45 (m, 3H), 2.86-2.67 (m, 2H), 2.32-2.19 (m, 5H), 2.12-2.04 (m, 2H), 1.71-1.62 (m, 2H), 1.56-1.34 (m, 7H), 1.23 (s, 1H), 1.17 (d, J=1.8 Hz, 1H), 1.16-1.12 (m, 3H), 1.01 (s, 6H). LC-MS (ESI⁺) m/z 644.2 (M+H)⁺.

### Step 2—6-[[4-[4-[2-(4-Chlorophenyl)-5,5-dimethyl-cyclohexen-1-yl]methyl]piperazin-1-yl]benzoyl]sulfamoyl]hexanoic acid

To a solution of ethyl 6-[[4-[4-[[2-(4-chlorophenyl)-5,5-dimethyl-cyclohexen-1-yl]methyl]piperazin-1-yl]benzoyl]sulfamoyl]hexanoate (100 mg, 155 umol) in MeOH (1 mL) and H₂O (0.5 mL) was added LiOH·H₂O (32.5 mg, 776 umol), then the reaction was stirred at 25° C., for 2 hrs. On completion, the reaction mixture was concentrated in vacuo, and diluted with water (5 mL), the mixture was acidified with HCl (4 N) till pH=6. Then the mixture was extracted with EtOAc (10 mL×2), the combined organic layers were washed with saturated brine, dried over Na₂SO₄, filtered and concentrated in vacuo to give the title compound (90 mg, 94% yield) as white solid. ¹H NMR (400 MHZ, DMSO-d₆) δ 12.05-11.92 (m, 1H), 11.87-11.05 (m, 1H), 7.81 (d, J=8.8 Hz, 2H), 7.39 (d, J=8.4 Hz, 2H), 7.14 (d, J=8.4 Hz, 2H), 6.94 (d, J=8.8 Hz, 2H), 3.51-3.45 (m, 2H), 2.52 (d, J=1.6 Hz, 7H), 2.25 (s, 2H), 2.22-2.13 (m, 3H), 2.03 (s, 2H), 1.71-1.62 (m, 2H), 1.54-1.33 (m, 7H), 0.99 (s, 6H), 0.92-0.77 (m, 1H) LC-MS (ESI⁺) m/z 616.2 (M+H)⁺.

### Ethyl 8-sulfamoyloctanoate (Intermediate CV)

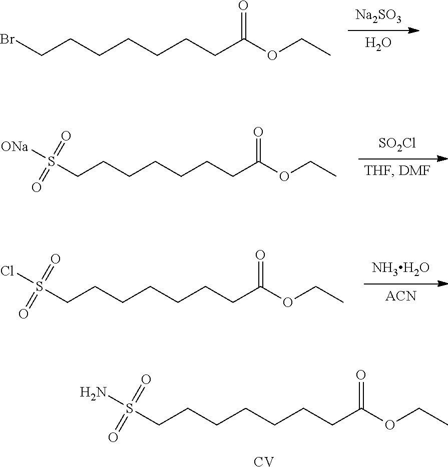

### Step 1—(8-Ethoxy-8-oxo-octyl)sulfonylsodium

A solution of ethyl 8-bromooctanoate (5.00 g, 19.9 mmol) and Na₂SO; (3.26 g, 25.9 mmol) in H₂O (25 mL) was stirred at 120° C., for 12 h. On completion, the mixture was cooled and lyophilized to give the title compound (5.20 g) as a white solid. ¹H NMR (400 MHZ, DMSO-d₆) δ 4.02 (br d, J=6.4 Hz, 2H), 2.44 (br d, J=6.4 Hz, 2H), 2.24 (br s, 2H), 1.60-1.46 (m, 4H), 1.23 (br s, 6H), 1.15 (br t, J=6.4 Hz, 3H). Step 2—Ethyl 8-(chlorosulfonyl)octanoate

A solution of (8-ethoxy-8-oxo-octyl)sulfonylsodium (5.00 g, 19.4 mmol) in THF (50 mL) and DMF (2.5 mL) was added SOCl₂ (19.9 g, 167 mmol, 12.2 mL) dropwise at 0° C., under N₂ and the mixture was heated to 70° C., and stirred for 1 h. On completion, the mixture was concentrated in vacuo to give the title compound (5.24 g) as a white solid.

### Step 3—Ethyl 8-sulfamoyloctanoate

Ethyl 8-(chlorosulfonyl)octanoate (5.24 g, 19.35 mmol) in DMF (10.5 mL) was diluted with ACN (50 mL). The resulting suspension was added into NH₃·H₂O (34.1 g, 243 mmol, 25% solution) at 0° C. under N₂ and stirred for 30 min. On completion, the reaction mixture was diluted with EtOAc (15 mL). The combined organic layers were washed with H₂O (5 mL×1), brine (5 mL×1), dried over Na₂SO₄ and evaporated. The residue was purified by column chromatography (SiO₂, Petroleum ether:Ethyl acetate=3/1) to give the title compound (3.70 g, 61% yield) as a yellow oil. ¹H NMR (400 MHZ, CDCl₃) δ 4.72 (br s, 2H), 4.12 (q, J=7.2 Hz, 2H), 3.15-3.08 (m, 2H), 2.30 (t, J=7.2 Hz, 2H), 1.88-1.82 (m, 2H), 1.63 (br t, J=7.2 Hz, 2H), 1.51-1.42 (m, 2H), 1.39-1.32 (m, 4H), 1.26 (t, J=7.2 Hz, 3H).

### 8-[[4-[4-[[2-(4-chlorophenyl)-5,5-dimethyl-cyclohexen-1-yl]methyl]piperazin-1-yl]benzoyl]sulfamoyl]octanoic acid (Intermediate CW)

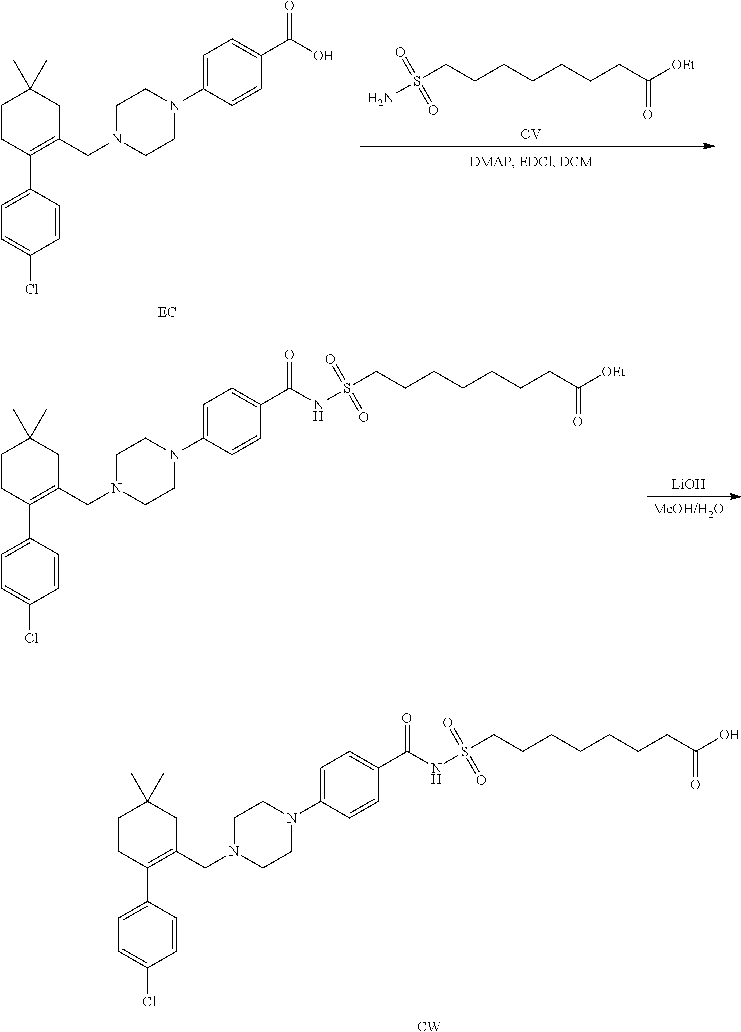

### Step 1—Ethyl 8-[[4-[4-[[2-(4-chlorophenyl)-5,5-dimethyl-cyclohexen-1-yl]methyl]piperazin-1-yl]benzoyl]sulfamoyl]octanoate

To a solution of 4-[4-[[2-(4-chlorophenyl)-5,5-dimethyl-cyclohexen-1-yl]methyl]piperazin-1-yl]benzoic acid (300 mg, 683 umol, Intermediate EC), EDCI (262 mg, 1.37 mmol) and DMAP (166 mg, 1.37 mmol) in DCM (5 mL) was added ethyl 8-sulfamoyloctanoate (429 mg, 1.37 mmol, Intermediate CV). The reaction mixture was then stirred at 25° C., for 12 hrs. On completion, the reaction mixture was diluted with water (15 mL), then extracted with DCM (50 mL×2). The combined organic layers were washed with brine (15 mL), dried over Na₂SO₄ and evaporated. The residue was purified by column chromatography (SiO₂, PE:EA=3:1, DCM:MeOH=10:1). Then the crude product was purified by prep HPLC (column: Phenomenex Luna C18 200*40 mm*10 um; mobile phase: [water (FA)-ACN]; B %: 33%-63%, 10 min) to give the title compound (310 mg, 68% yield) as a white solid. ¹H NMR (400 MHZ, DMSO-d₆) δ 11.89-11.25 (m, 1H), 7.79 (d, J=9.0 Hz, 2H), 7.37 (d, J=8.4 Hz, 2H), 7.11 (d, J=8.4 Hz, 2H), 6.91 (d, J=9.0 Hz, 2H), 4.05-3.99 (m, 2H), 3.49-3.42 (m, 2H), 3.27 (br s, 2H), 2.78 (br s, 2H), 2.29 (br d, J=7.0 Hz, 4H), 2.22 (t, J=7.2 Hz, 4H), 1.99 (s, 2H), 1.65 (br dd, J=6.6, 8.6 Hz, 2H), 1.50-1.41 (m, 4H), 1.35 (br d, J=7.0 Hz, 2H), 1.30-1.19 (m, 6H), 1.15 (t, J=7.0 Hz, 3H), 0.96 (s, 6H).

### Step 2—8-[4-[4-[2-(4-Chlorophenyl)-5,5-dimethyl-cyclohexen-1-yl]methyl]piperazin-1-yl]benzoyl]sulfamoyl]octanoic acid

To a solution of ethyl ethyl 8-[[4-[4-[2-(4-chlorophenyl)-5,5-dimethyl-cyclohexen-1-yl]methyl]piperazin-1-yl]benzoyl]sulfamoyl]octanoate (300 mg, 446 umol) in MeOH (3 mL) and H₂O (1 mL) was added LiOH (53.4 mg, 2.23 mmol), then the reaction mixture was stirred at 40° C., for 2 hrs. On completion, the reaction mixture was diluted with EA (10 mL), then extracted with EA (15 mL×3). The combined organic layers were washed with brine (10 mL×3), dried over Na₂SO₄ and evaporated to give the title compound (278 mg, 96% yield) as a yellow solid. ¹H NMR (400 MHZ, DMSO-d₆) δ 12.10-11.84 (m, 1H), 11.66-11.36 (m, 1H), 7.79 (d, J=9.0 Hz, 2H), 7.37 (d, J=8.4 Hz, 2H), 7.12 (d, J=8.4 Hz, 2H), 6.91 (d, J=9.0 Hz, 2H), 3.48-3.41 (m, 2H), 3.27 (br s, 4H), 2.78 (s, 2H), 2.29 (br s, 4H), 2.20-2.12 (m, 3H), 2.04-1.95 (m, 2H), 1.66 (quin, J=7.5 Hz, 3H), 1.49-1.41 (m, 4H), 1.30-1.18 (m, 6H), 0.97 (s, 6H).

### 2-(8-(benzo[d]thiazol-2-ylcarbamoyl)-3,4-dihydroisoquinolin-2 (1H)-yl) thiazole-4-carboxylic acid (Intermediate CX)

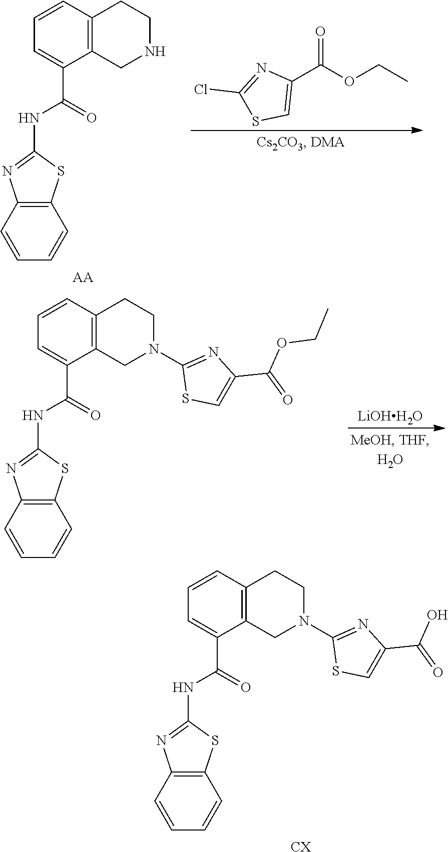

### Step 1—Ethyl 2-(8-(benzo[d]thiazol-2-ylcarbamoyl)-3,4-dihydroisoquinolin-2 (1H)-yl) thiazole-4-carboxylate

To a solution of N-(benzo[d]thiazol-2-yl)-1,2,3,4-tetrahydroisoquinoline-8-carboxamide (3 g, 8.67 mmol, HCl, Intermediate AA) and ethyl 2-chlorothiazole-4-carboxylate (2.66 g, 13.9 mmol) in DMA (50 mL) was added Cs₂CO₃ (22.6 g, 69.4 mmol). The mixture was stirred at 50° C., for 12 h. On completion, The reaction mixture was poured into ice water (50 mL), and extracted with EtOAc (50 mL×3). The combined organic layers were washed with brine (100 mL×3), dried over sodium sulfate, filtered and concentrated under reduced pressure to give a residue. The residue was purified by flash silica gel chromatography (ISCOR: 40 g SepaFlash® Silica Flash Column, Eluent of 0˜30% Ethyl acetate/Petroleum ethergradient @80 mL/min) to give the title compound (3.6 g, 89% yield) as a yellow oil. ¹H NMR (400 MHz, DMSO-d₆) δ 8.04 (d, J=7.8 Hz, 1H), 7.79 (d, J=8.0 Hz, 1H), 7.71-7.64 (m, 2H), 7.51-7.43 (m, 2H), 7.42-7.33 (m, 2H), 4.89 (s, 2H), 4.20 (q, J=7.2 Hz, 2H), 3.76 (t, J=6.0 Hz, 2H), 3.06 (br t, J=6.0 Hz, 2H), 1.22 (t, J=7.2 Hz, 3H).

### Step 2—2-(8-(Benzo[d]thiazol-2-ylcarbamoyl)-3,4-dihydroisoquinolin-2 (1H)-yl) thiazole-4-carboxylic acid

To a solution of ethyl 2-(8-(benzo[d]thiazol-2-ylcarbamoyl)-3,4-dihydroisoquinolin-2 (1H)-yl) thiazole-4-carboxylate (3.5 g, 7.53 mmol) in THF (12 mL), H₂O (12 mL) and MeOH (12 mL) was added LiOH·H₂O (1.26 g, 30.1 mmol). The mixture was stirred at 25° C., for 12 h. On completion, the reaction mixture was poured into water (40 mL) and extracted with EtOAc (40×3 mL). The aqueous phase was acidified with 1N HCl to pH=4, then extracted with EtOAc (40×3 mL). The combined organic layers were washed with brine (50×3 mL), dried over Na₂SO₄, filtered and concentrated under reduced pressure to give the title compound (3 g) as a yellow solid. ¹H NMR (400 MHZ, DMSO-d₆) δ 7.34 (dd, J=3.2, 5.6 Hz, 1H), 7.21 (d, J=7.8 Hz, 1H), 7.07 (d, J=8.0 Hz, 1H), 6.83-6.70 (m, 3H), 6.65-6.53 (m, 2H), 4.66 (s, 2H), 3.19 (br s, 3H), 2.50 (br t, J=6.0 Hz, 2H)

### (R)-2-(8-(benzo[d]thiazol-2-ylcarbamoyl)-3,4-dihydroisoquinolin-2 (1H)-yl)-N-((4-((1-(phenylthio)-4-(piperazin-1-yl)butan-2-yl)amino)-3-((trifluoromethyl)sulfonyl)phenyl)sulfonyl) thiazole-4-carboxamide (Intermediate CY)

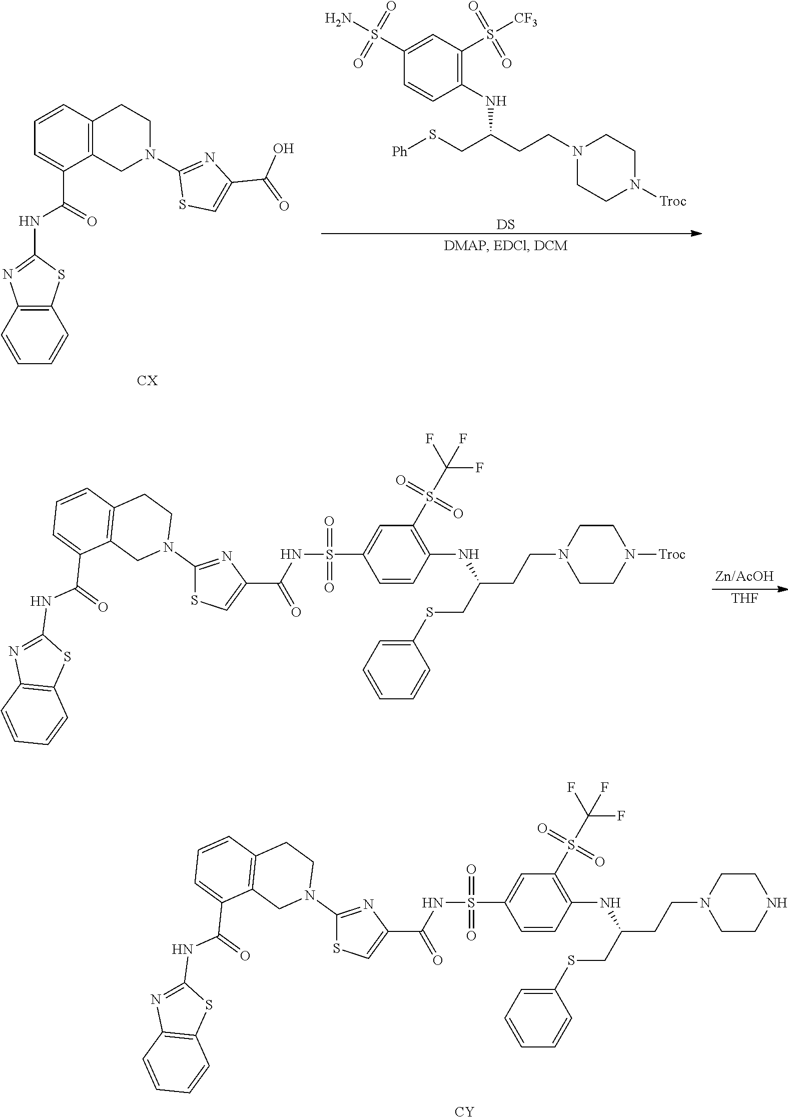

### Step 1—(R)-2,2,2-trichloroethyl 4-(3-((4-(N-(2-(8-(benzo[d]thiazol-2-ylcarbamoyl)-3,4-dihydroisoquinolin-2 (1H)-yl) thiazole-4-carbonyl)sulfamoyl)-2-((trifluoromethyl)sulfonyl)phenyl)amino)-4-(phenylthio)butyl)piperazine-1-carboxylate

To a solution of 2-(8-(benzo[d]thiazol-2-ylcarbamoyl)-3,4-dihydroisoquinolin-2 (1H)-yl) thiazole-4-carboxylic acid (100 mg, 229 umol, Intermediate CX) and (R)-2,2,2-trichloroethyl4-(4-(phenylthio)-3-((4-sulfamoyl-2-((trifluoromethyl)sulfonyl)phenyl)amino)butyl)piperazine-1-carboxylate (167 mg, 229 umol, Intermediate DS) in DCM (2 mL) was added EDCI (220 mg, 1.15 mmol) and DMAP (140 mg, 1.15 mmol). The mixture was stirred at 25° C., for 2 h. On completion, the reaction mixture was poured into ice water (5 mL) and extracted with EtOAc (5 mL×3). The combined organic layers were washed with brine (5 mL×3), dried over sodium sulfate, filtered and concentrated under reduced pressure to give a residue. The residue was purified by column chromatography (SiO₂, Petroleum ether/Ethyl acetate=0/1 to DCM:MeOH=10/1) to give the title compound (200 mg, 53% yield) as a white oil. LC-MS (ESI⁺) m/z 1144.07 (M+H)⁺.

### Step 2—(R)-2-(8-(benzo[d]thiazol-2-ylcarbamoyl)-3,4-dihydroisoquinolin-2 (1H)-yl)-N-((4-((1-(phenylthio)-4-(piperazin-1-yl)butan-2-yl)amino)-3-((trifluoromethyl)sulfonyl)phenyl)sulfonyl) thiazole-4-carboxamide

To a solution of (R)-2,2,2-trichloroethyl 4-(3-((4-(N-(2-(8-(benzo[d]thiazol-2-ylcarbamoyl)-3,4-dihydroisoquinolin-2 (1H)-yl) thiazole-4-carbonyl)sulfamoyl)-2-((trifluoromethyl)sulfonyl)phenyl)amino)-4-(phenylthio)butyl)piperazine-1-carboxylate (200 mg, 174 umol), CH₃COOH (400 mg, 6.66 mmol, 381 uL) in THF (4 mL) was added Zn (700 mg, 10.7 mmol). The mixture was stirred at 20° C., for 12 h. On completion, the solid was removed by filtration, and the filtrate was poured into H₂O (10 mL) at 25° C., and extracted with EtOAc (5 mL×3). The combined organic layers were washed with brine (5 mL×3), dried over sodium sulfate, filtered and concentrated under reduced pressure to give the title compound (250 mg) as a white solid. LC-MS (ESI⁺) m/z 971.2 (M+H)⁺.

### 6-(8-(Benzo[d]thiazol-2-ylcarbamoyl)-3,4-dihydroisoquinolin-2 (1H)-yl)picolinic acid (Intermediate CZ)

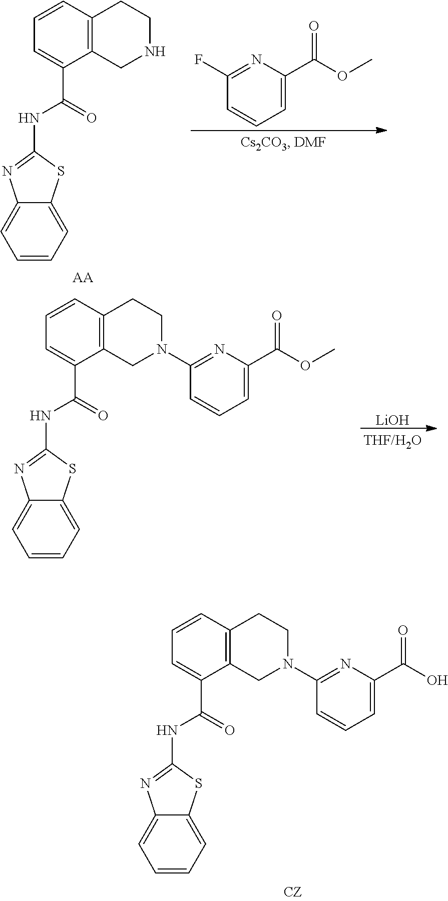

### Step 1—Methyl 6-(8-(benzo[d]thiazol-2-ylcarbamoyl)-3,4-dihydroisoquinolin-2 (1H)-yl)picolinate

To a solution of N-(benzo[d]thiazol-2-yl)-1,2,3,4-tetrahydroisoquinoline-8-carboxamide (2.19 g, 7.09 mmol, Intermediate AA), methyl 6-fluoropicolinate (1.00 g, 6.45 mmol, CAS #455-71-0) in DMF (15 mL) was added Cs₂CO₃ (8.4 g, 25.8 mmol), then the mixture was stirred at 100° C., for 12 h. On completion, the reaction mixture was quenched with NH₄Cl (10 mL) and extracted with EtOAc (15 mL×2). The combined organic layers were dried over Na₂SO₄ and evaporated. The residue was purified by silica gel column chromatography (Petroleum ether:Ethyl acetate=10:1 to 3:1) to give the title compound (560 mg, 20% yield) as a white solid. ¹H NMR (400 MHZ, DMSO-d₆) δ 7.88 (d, J=7.6 Hz, 1H), 7.67 (br t, J=8.0 Hz, 2H), 7.56 (t, J=8.0 Hz, 1H), 7.41-7.07 (m, 5H), 6.82 (d, J=8.4 Hz, 1H), 5.02 (s, 2H), 3.89 (br t, J=5.6 Hz, 3H), 3.72 (br s, 2H), 3.17 (s, 1H), 2.95 (br t, J=5.6 Hz, 2H).

### Step 2—6-(8-(Benzo[d]thiazol-2-ylcarbamoyl)-3,4-dihydroisoquinolin-2 (1H)-yl)picolinic acid

To a solution of methyl 6-(8-(benzo[d]thiazol-2-ylcarbamoyl)-3,4-dihydroisoquinolin-2 (1H)-yl)picolinate (500 mg, 787 umol) in THF (4 mL), H₂O (1 mL) was added to LiOH·H₂O (99.1 mg, 2.36 mmol), then the mixture was stirred at 25° C., for 17 h. On completion, the reaction mixture was filtered and the pH of the aqueous phase was adjusted to 4˜5 by addition 2M HCl and extracted with EtOAC (5 mL×2). The combined organic layers were dried over Na₂SO₄ and evaporated to give the title compound (500 mg) as an orange solid. LC-MS (ESI⁺) m/z 431.2 (M+H)⁺.

### 6-(N-(6-(8-(benzo[d]thiazol-2-ylcarbamoyl)-3,4-dihydroisoquinolin-2 (1H)-yl)picolinoyl)sulfamoyl)hexanoic acid (Intermediate DA)

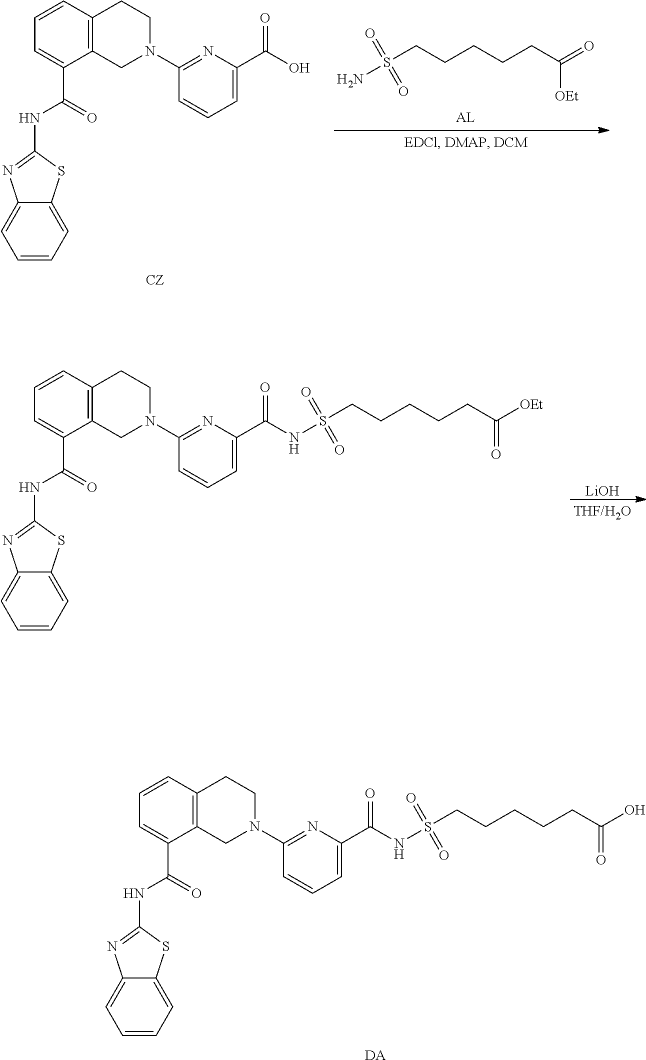 [SMILES: O=C(O)c1cccc(N2CCc3cccc(C(=O)Nc4nc5ccccc5s4)c3C2)n1 ; CCOC(=O)CCCCCS(N)(=O)=O ; CCOC(=O)CCCCCS(=O)(=O)NC(=O)c1cccc(N2CCc3cccc(C(=O)Nc4nc5ccccc5s4)c3C2)n1 ; "LiOH" ; O=C(O)CCCCCS(=O)(=O)NC(=O)c1cccc(N2CCc3cccc(C(=O)Nc4nc5ccccc5s4)c3C2)n1 ; "DA"]

### Step 1—Ethyl 6-(N-(6-(8-(benzo[d]thiazol-2-ylcarbamoyl)-3,4-dihydroisoquinolin-2 (1H)-yl)picolinoyl)sulfamoyl)hexanoate

To a solution of 6-(8-(benzo[d]thiazol-2-ylcarbamoyl)-3,4-dihydroisoquinolin-2 (1H)-yl)picolinic acid (303 mg, 563 umol, Intermediate CZ), ethyl 6-sulfamoylhexanoate (252 mg, 1.13 mmol, Intermediate AL) in DCM (5 mL) was added EDCI (540 mg, 2.82 mmol), DMAP (344 mg, 2.82 mmol), then the mixture was stirred at 25° C., for 17 h. On completion, the solution was concentrated in vacuo. The mixture was further purification by pre-HPLC (column: Phenomenex C18 150*25 mm*10 um; mobile phase: [water (NH₄HCO₃)-ACN]: B %: 30%-60%, 8 min) to give the title compound (50 mg, 14% yield) as an orange solid. LC-MS (ESI⁺) m/z 636.4 (M+H)⁺.

### Step 2—6-(N-(6-(8-(benzo[d]thiazol-2-ylcarbamoyl)-3,4-dihydroisoquinolin-2 (1H)-yl)picolinoyl)sulfamoyl)hexanoic acid

To a solution of ethyl 6-(N-(6-(8-(benzo[d]thiazol-2-ylcarbamoyl)-3,4-dihydroisoquinolin-2 (1H)-yl)picolinoyl)sulfamoyl)hexanoate (50.0 mg, 78.7 umol) in THF (1 mL) and H₂O (0.25 mL) was added LiOH·H₂O (33.0 mg, 787 umol), the mixture was then stirred at 25° C., for 12 h. On completion, the reaction mixture was filtered and the pH of the aqueous phase was adjusted to 4˜5 by addition 2M HCl and extracted with EtOAC (2 mL×2). The combined organic layers were dried over Na₂SO₄ and evaporated to afford the title compound (35.0 mg) as an orange solid. LC-MS (ESI⁺) m/z 608.2 (M+H)⁺.

### Tert-butyl 3-bromo-6-(methylamino)picolinate (Intermediate DB)

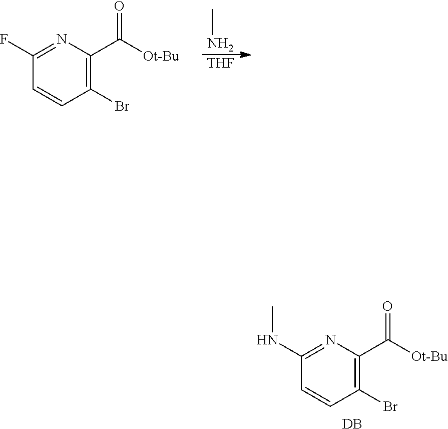

A mixture of tert-butyl 3-bromo-6-fluoro-pyridine-2-carboxylate (2 g, 7.24 mmol), and methanamine (2 M, 35.6 mL, 8% solution) in THF (30 mL) was stirred at 60° C., for 4 h. On completion, the reaction mixture was filtered and concentrated under reduced pressure to give a residue. The residue was purified by column chromatography (SiO₂, Petroleum ether/Ethyl acetate=20/1 to 5/1) to give the title compound (1.6 g, 77% yield) as a white solid. LC-MS (ESI⁺) m/z 287.1 (M+H)⁺.

### 6-((6-(benzo[d]thiazol-2-ylamino)-5-methylpyridazin-3-yl)(methyl)amino)-3-(1-(cyclohexylmethyl)-5-methyl-1H-pyrazol-4-yl)picolinic acid (Intermediate DC)

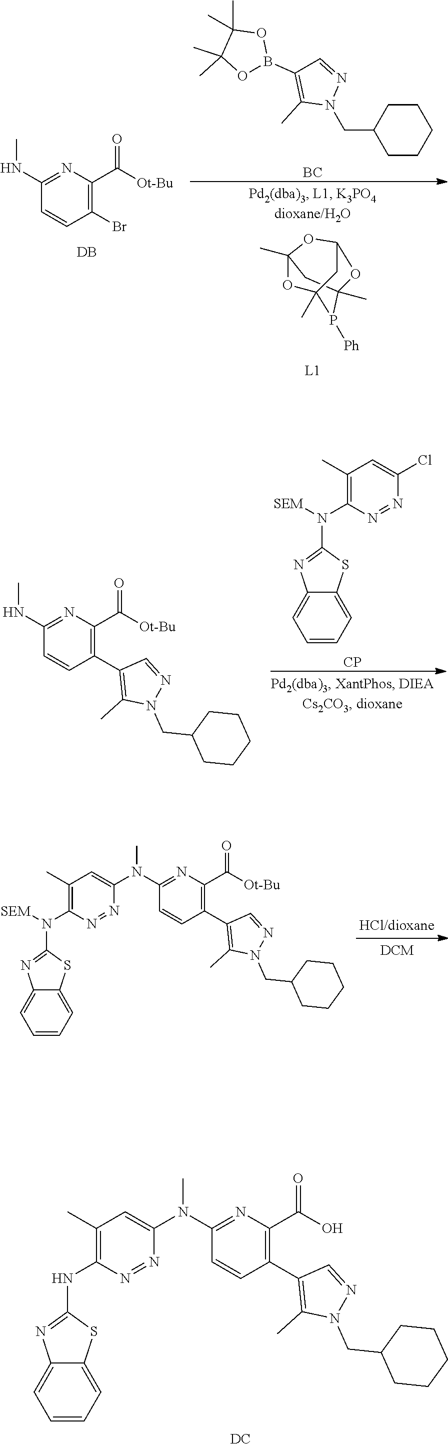

### Step 1—Tert-butyl 3-(1-(cyclohexylmethyl)-5-methyl-1H-pyrazol-4-yl)-6-(methylamino)picolinate

A mixture of tert-butyl 3-bromo-6-(methylamino)pyridine-2-carboxylate (1.1 g, 3.83 mmol, Intermediate DB) and 1-(cyclohexylmethyl)-5-methyl-4-(4,4,5,5-tetramethyl-1,3,2-dioxaborolan-2-yl) pyrazole (1.17 g, 3.83 mmol, Intermediate BC) in dioxane (30 mL) and H₂O (20 mL) was added to K₃PO₄ (2.44 g, 11.5 mmol), (1S,3R,5R,7S)-1,3,5,7-tetramethyl-8-phenyl-2,4,6-trioxa-8-phosphatricyclo[3,3,1,1^{3,7}]decane (224 mg, 766 umol), and (1E,4E)-1,5-diphenylpenta-1,4-dien-3-one; palladium (175 mg, 191 umol). Then the mixture was degassed and purged with N₂ three times, and then the mixture was stirred at 100° C., for 2 h under N₂ atmosphere. On completion, the reaction mixture was quenched with H₂O (5 mL), and extracted with EtOAc (15 mL×3). The combined organic layer was washed with brine (15 mL×2), dried over Na₂SO₄, filtered and concentrated. The residue was purified by column chromatography (SiO₂, Petroleum ether/Ethyl acetate=10/1 to 5/1 to give the title compound (1.45 g, 98% yield) as a yellow gum. LC-MS (ESI⁺) m/z 385.5 (M+H)⁺. ¹H NMR (400 MHZ, DMSO-d₆) δ=7.26 (d, J=8.4 Hz, 1H), 7.19 (s, 1H), 6.70 (br d, J=4.8 Hz, 1H), 6.53 (d, J=8.4 Hz, 1H), 3.86 (d, J=7.2 Hz, 2H), 2.77 (d, J=4.8 Hz, 3H), 2.08 (s, 3H), 1.70-1.53 (m, 6H), 1.40 (s, 1H), 1.27 (s, 9H), 1.03-0.92 (m, 2H).

### Step 2—Tert-butyl 6-((6-(benzo[d]thiazol-2-yl((2-(trimethylsilyl)ethoxy)methyl)amino)-5-methylpyridazin-3-yl)(methyl)amino)-3-(1-(cyclohexylmethyl)-5-methyl-1H-pyrazol-4-yl)picolinate

A mixture of tert-butyl 6-((6-(benzo[d]thiazol-2-yl((2-(trimethylsilyl)ethoxy)methyl)amino)-5-methylpyridazin-3-yl)(methyl)amino)-3-(1-(cyclohexylmethyl)-5-methyl-1H-pyrazol-4-yl)picolinate (2.89 g, 7.10 mmol), N-(6-chloro-4-methylpyridazin-3-yl)-N-((2-(trimethylsilyl)ethoxy)methyl)benzo[d]thiazol-2-amine (2.89 g, 7.10 mmol, Intermediate CP), (1E,4E)-1,5-diphenylpenta-1,4-dien-3-one: palladium (500.11 mg, 546.14 umol), dicesium: carbonate (5.34 g, 16.4 mmol), DIEA (2.12 g, 16.4 mmol, 2.85 mL) and (5-diphenylphosphanyl-9,9-dimethyl-xanthen-4-yl)-diphenyl-phosphane (632 mg, 1.09 mmol) in dioxane (60 mL) was degassed and purged with N₂ three times. Then the mixture was stirred at 120° C., for 12 h under N₂ atmosphere. On completion, the reaction mixture was quenched with H₂O (50 mL), and extracted with EtOAc (50 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=5/1 to 3/1) to give the title compound (3.5 g, 85% yield) as a brown solid. LC-MS (ESI⁺) m/z 755.06 (M+H).

### Step 3—6-((6-(Benzo[d]thiazol-2-ylamino)-5-methylpyridazin-3-yl)(methyl)amino)-3-(1-(cyclohexylmethyl)-5-methyl-1H-pyrazol-4-yl)picolinic acid

A mixture of tert-butyl 6-((6-(benzo[d]thiazol-2-yl((2-(trimethylsilyl)ethoxy)methyl)amino)-5-methylpyridazin-3-yl)(methyl)amino)-3-(1-(cyclohexylmethyl)-5-methyl-1H-pyrazol-4-yl)picolinate (3.5 g, 4.64 mmol) in DCM (20 mL) was added to HCl/dioxane (4 M, 30 mL), and then the mixture was stirred at 40° C., for 4 h. On completion, the reaction mixture was filtered and concentrated under reduced pressure to give the title product (2.5 g) as a brown solid. LC-MS (ESI⁺) m/z 569.4 (M+H)⁺.

### 6-(N-(6-((6-(benzo[d]thiazol-2-ylamino)-5-methylpyridazin-3-yl)(methyl)amino)-3-(1-(cyclohexylmethyl)-5-methyl-1H-pyrazol-4-yl)picolinoyl)sulfamoyl)hexanoic acid (Intermediate DD)

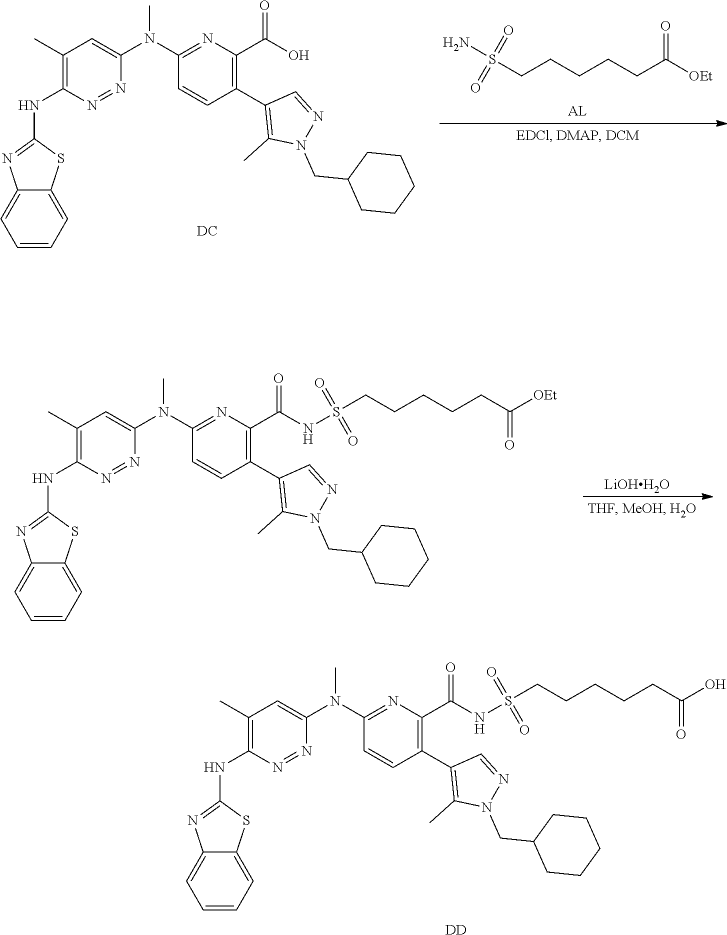

### Step 1—Ethyl 6-(N-(6-((6-(benzo[d]thiazol-2-ylamino)-5-methylpyridazin-3-yl)(methyl)amino)-3-(1-(cyclohexylmethyl)-5-methyl-1H-pyrazol-4-yl)picolinoyl)sulfamoyl)hexanoate

To a solution of 6-((6-(benzo[d]thiazol-2-ylamino)-5-methylpyridazin-3-yl)(methyl)amino)-3-(1-(cyclohexylmethyl)-5-methyl-1H-pyrazol-4-yl)picolinic acid (500 mg, 879 umol, Intermediate DC) and ethyl 6-sulfamoylhexanoate (392 mg, 1.76 mmol, Intermediate AL) in DCM (3 mL) was added DMAP (537 mg, 4.40 mmol) and EDCI (842 mg, 4.40 mmol). The mixture was stirred at 25° C., for 24 h. On completion, the reaction mixture was quenched with H₂O (20 mL), and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=1/1 to DCM:MeOH=10:1) to give the title compound (430 mg, 63% yield) as a brown solid. ¹H NMR (400 MHZ, DMSO-d₆) δ=8.11 (br d, J=6.4 Hz, 4H), 7.86 (br d, J=7.6 Hz, 1H), 7.69 (s, 1H), 7.55 (s, 1H), 7.50 (s, 1H), 7.38 (br t, J=7.6 Hz, 1H), 7.20 (br t, J=7.6 Hz, 1H), 7.12 (d, J=8.4 Hz, 1H), 6.68 (br d, J=6.4 Hz, 4H), 4.01 (q, J=7.2 Hz, 2H), 3.85 (br d, J=7.2 Hz, 2H), 3.65-3.60 (m, 3H), 2.98-2.92 (m, 2H), 2.34 (s, 2H), 2.24-2.21 (m, 3H), 1.81 (ddd, J=3.2, 7.2, 10.4 Hz, 1H), 1.68-1.56 (m, 4H), 1.49 (dt, J=7.6, 15.7 Hz, 4H), 1.26 (br dd, J=7.6, 15.2 Hz, 2H), 1.20-1.11 (m, 5H), 1.03-0.94 (m, 2H).

### Step 2—6-(N-(6-((6-(benzo[d]thiazol-2-ylamino)-5-methylpyridazin-3-yl)(methyl)amino)-3-(1-(cyclohexylmethyl)-5-methyl-1H-pyrazol-4-yl)picolinoyl)sulfamoyl)hexanoic acid

A mixture of ethyl 6-(N-(6-((6-(benzo[d]thiazol-2-ylamino)-5-methylpyridazin-3-yl)(methyl)amino)-3-(1-(cyclohexylmethyl)-5-methyl-1H-pyrazol-4-yl)picolinoyl)sulfamoyl)hexanoate (0.43 g, 555 umol) in H₂O (1 mL), THF (1 mL) and MeOH (0.5 mL) was added LiOHH₂O (233 mg, 5.56 mmol). The mixture was stirred at 25° C., for 2 h. On completion, the reaction mixture was concentrated under reduced pressure, and adjusted pH to 5˜6 with 2M HCl, then extracted with EtOAc (5 mL×3). The combined organic layers were washed with brine (5 mL×2), dried over Na₂SO₄, filtered and concentrated under reduced pressure to give the title compound (0.25 g) as brown solid. LC-MS (ESI⁺) m/z 746.5 (M+H)⁺.

### 1-(3-(1-((4-iodo-5-methyl-1H-pyrazol-1-yl)methyl)cyclohexyl)propyl)piperazine (Intermediate DE)

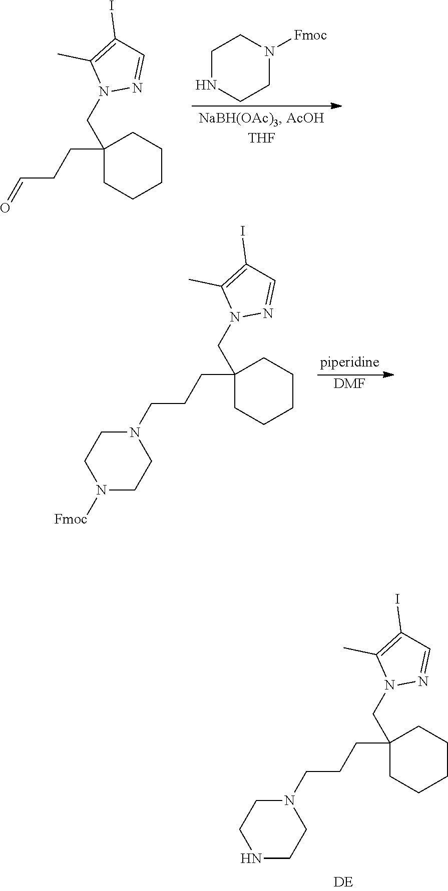

### Step 1—(9H-fluoren-9-yl)methyl 4-(3-(1-((4-iodo-5-methyl-1H-pyrazol-1-yl)methyl)cyclohexyl)propyl)piperazine-1-carboxylate

To a solution of 3-(1-((4-iodo-5-methyl-1H-pyrazol-1-yl)methyl)cyclohexyl)propanal (2.30 g, 5.11 mmol, synthesized via Steps 1-7 of Intermediate BR) and (9H-fluoren-9-yl)methyl piperazine-1-carboxylate (3.15 g, 10.2 mmol) in THF (23 mL) was added to NaBH (OAc); (2.17 g, 10.2 mmol) and AcOH (2.42 g, 40.22 mmol), them the mixture was stirred at 25° C., for 2 h. On completion, the reaction mixture was quenched with sat. NaHCO₃ (15 mL), and then extracted with EtOAc (20 mL×3). The combined organic layers were washed with brine (20 mL×3), dried over Na₂SO₄, filtered and concentrated under reduced pressure to give a residue. The residue was purified by flash silica gel chromatography (Ethyl acetate/Petroleum ether=0/1 to 1/5) to give the title compound (3.00 g, 72% yield) as a yellow oil. 1H NMR (400 MHZ, DMSO-d₆) δ=7.88 (d, J=7.6 Hz, 2H), 7.62 (d, J=7.6 Hz, 2H), 7.45 (s, 1H), 7.43-7.38 (m, 2H), 7.36-7.31 (m, 2H), 4.37 (d, J=6.4 Hz, 2H), 4.29-4.23 (m, 1H), 4.02 (q, J=7.2 Hz, 2H), 3.92 (s, 2H), 3.60 (t, J=6.4 Hz, 1H), 2.33-2.11 (m, 10H), 1.46 (d, J=2.8 Hz, 4H), 1.36-1.22 (m, 10H),

### Step 2—1-(3-(1-((4-Iodo-5-methyl-1H-pyrazol-1-yl)methyl)cyclohexyl)propyl)piperazine

To a solution of (9H-fluoren-9-yl)methyl 4-(3-(1-((4-iodo-5-methyl-1H-pyrazol-1-yl)methyl)cyclohexyl)propyl)piperazine-1-carboxylate (1.00 g, 1.53 mmol) in DMF (9 mL) was added to piperidine (2.59 g, 30.3 mmol), then the mixture was stirred at 25° C., for 0.5 h. On completion, the reaction mixture was quenched with H₂O (10 mL), and then extracted with EtOAc (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=10/1 to DCM:MeOH=10:1) to give the title compound (380 mg, 48% yield) as a yellow solid. ¹H NMR (400 MHZ, DMSO-d₆) δ=7.44 (s, 1H), 3.93 (s, 2H), 2.96-2.89 (m, 2H), 2.78 (t, J=4.8 Hz, 4H), 2.26 (s, 3H), 2.18 (d, J=7.2 Hz, 2H), 1.59 (d, J=4.8 Hz, 2H), 1.46 (d, J=4.0 Hz, 2H), 1.33-1.17 (m, 12H).

### (2S,4R)-4-hydroxy-1-((S)-2-(5-(4-(3-(1-((4-iodo-5-methyl-1H-pyrazol-1-yl)methyl)cyclohexyl)propyl)piperazin-1-yl)-5-oxopentanamido)-3,3-dimethylbutanoyl)-N-((S)-1-(4-(4-methylthiazol-5-yl)phenyl)ethyl)pyrrolidine-2-carboxamide (Intermediate DF)

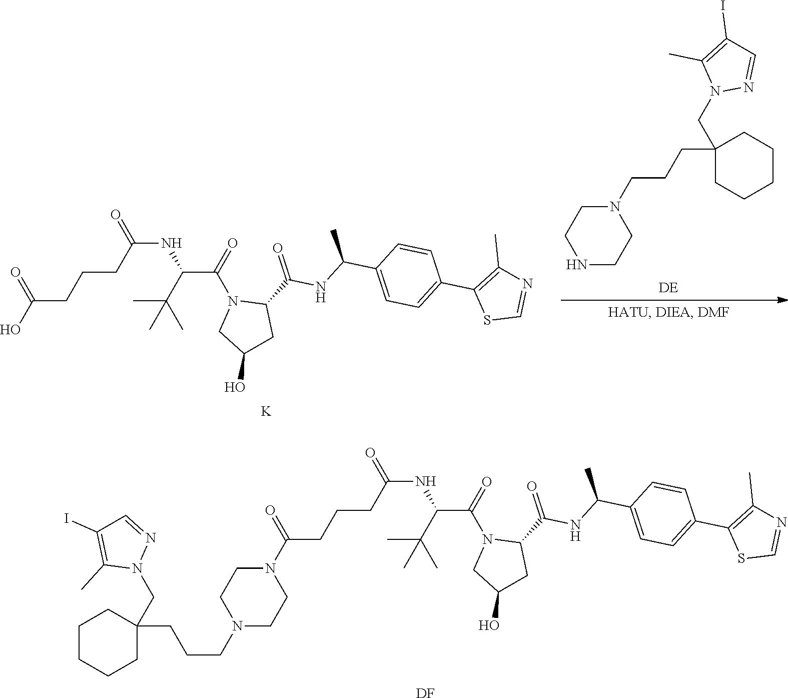

To a solution of 1-(3-(1-((4-iodo-5-methyl-1H-pyrazol-1-yl)methyl)cyclohexyl)propyl)piperazine (330 mg, 766 umol, Intermediate DE) and 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (471 mg, 843 umol, Intermediate K) in DMF (2 mL) was added to HATU (437 mg, 1.15 mmol) and DIEA (495 mg, 3.83 mmol), then the mixture was stirred at 25° C., for 2 h. On completion, the reaction mixture was quenched with sat. NH₄Cl (3 mL), and then extracted with EtOAc (5 mL×3). The combined organic layers were washed with brine (5 mL×3), dried over Na₂SO₄, filtered and concentrated under reduced pressure to give a residue. The residue was purified by column chromatography (SiO₂, DCM:MeOH=10:1) to give the title compound (300 mg, 35% yield) as a yellow solid. LC-MS (ESI⁺) m/z 971.5 (M+H)⁺: ¹H NMR (400 MHZ, DMSO-d₆) δ=8.98 (s, 1H), 8.37 (d, J=7.8 Hz, 1H), 7.95 (s, 1H), 7.82 (d, J=9.2 Hz, 1H), 7.48-7.35 (m, 5H), 5.75 (s, 1H), 5.10 (d, J=3.6 Hz, 1H), 4.95-4.85 (m, 1H), 4.53-4.38 (m, 2H), 4.32-4.24 (m, 1H), 3.94 (s, 2H), 3.61 (s, 3H), 3.13 (dd, J=4.0, 6.8 Hz, 2H), 2.45 (s, 3H), 2.27 (s, 6H), 2.24-2.14 (m, 4H), 2.05-1.93 (m, 2H), 1.80 (td, J=4.4, 8.4 Hz, 1H), 1.73-1.67 (m, 2H), 1.52-1.35 (m, 12H), 1.26 (s, 6H), 0.97-0.92 (m, 9H).

### Ethyl 5-sulfamoylpentanoate (Intermediate DG)

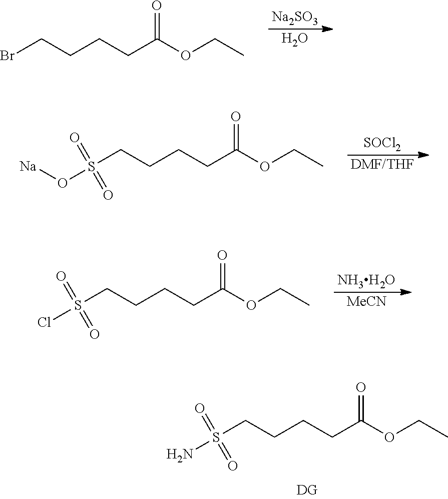

### Step 1—(5-Ethoxy-5-oxo-pentyl)sulfonyloxysodium

A mixture of ethyl 5-bromopentanoate (10.0 g, 47.8 mmol) and Na₂SO; (7.84 g, 62.2 mmol) in H₂O (50 mL) was degassed and purged with N₂ three times, and then the mixture was stirred at 120° C., for 12 h under N₂ atmosphere. On completion, the reaction mixture was lyophilized to give the title compound (10.0 g) as a white solid. ¹H NMR (400 MHZ, DMSO-d₆) δ=4.03 (q, J=7.2 Hz, 2H), 2.43 (br s, 2H), 2.26 (br s, 2H), 1.56 (br s, 4H), 1.17 (t, J=7.2 Hz, 3H).

### Step 2—Ethyl 5-chlorosulfonylpentanoate

A mixture of (5-ethoxy-5-oxo-pentyl)sulfonyloxysodium (10.0 g, 43.1 mmol) was dissolved in DMF (5 mL) and THF (100 mL). The mixture was cooled to 0° C., under N₂ and SOCl₂ (46.1 g, 387 mmol) was added dropwise. Then the mixture was heated to 70° C., and stirred for 1 h. On completion, the reaction mixture was concentrated under reduced pressure to give the title compound (10.0 g) as a yellow oil.

### Step 3—Ethyl 5-sulfamoylpentanoate

A mixture of ethyl 5-chlorosulfonylpentanoate (10.0 g, 43.7 mmol) was added to MeCN (100 mL). The resulting suspension was added into NH₃·H₂O (75 mL) at 0° C., and the mixture was stirred for 20 min. On completion, the reaction mixture was quenched with NH₄Cl (50 mL) and then extracted with EtOAc (60 mL×3). The combined organic layers were washed with brine (60 mL×2), dried over Na₂SO₄, filtered and concentrated under reduced pressure to give a residue. The residue was purified by column chromatography (SiO₂, Petroleum ether/Ethyl acetate=3/1 to 1/1) to give the title compound (8.50 g, 84% yield) as a yellow oil. ¹H NMR (400 MHZ, DMSO-d₆) δ=6.73 (s, 2H), 4.05 (q, J=7.2 Hz, 2H), 2.99-2.94 (m, 2H), 2.69 (s, 1H), 2.34-2.29 (m, 2H), 1.72-1.67 (m, 2H), 1.66-1.61 (m, 2H), 1.18 (t, J=7.2 Hz, 3H).

### 5-(N,N-bis(4-methoxybenzyl)sulfamoyl)pentyl 4-methylbenzenesulfonate (Intermediate DH)

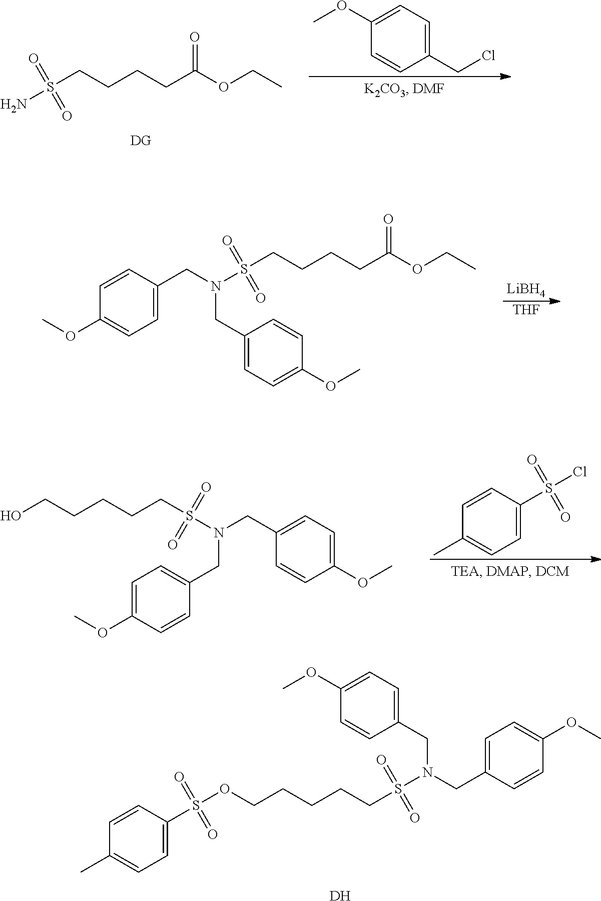

### Step 1—Ethyl 5-(N,N-bis(4-methoxybenzyl)sulfamoyl)pentanoate

To a mixture of 1-(chloromethyl)-4-methoxy-benzene (13.5 g, 86.0 mmol), ethyl 5-sulfamoylpentanoate (8.00 g, 34.4 mmol, Intermediate DG) in DMF (80 mL) was added and K₂CO₃ (19.0 g, 138 mmol) at 0° C., then the mixture was stirred at 50° C., for 16 h. On completion, the reaction mixture was quenched with H₂O (100 mL), then extracted with EtOAc (100 mL×3). The combined organic layers were washed with brine (100 mL×3), dried over Na₂SO₄, filtered and concentrated under reduced pressure to give a residue. The residue was purified by flash silica gel chromatography (Ethyl acetate/Petroleum ether=1/5 to 1/4) to give the title compound (12.0 g, 66% yield) as an white solid. ¹H NMR (400 MHZ, DMSO-d₆) ¿=7.18 (d, J=8.8 Hz, 4H), 6.89 (d, J=8.8 Hz, 4H), 4.20 (s, 4H), 4.09-4.01 (m, 4H), 3.74 (s, 6H), 3.06-3.02 (m, 2H), 2.29 (t, J=7.2 Hz, 2H), 1.99 (s, 2H), 1.67-1.57 (m, 4H), 1.18 (dt, J=2.8, 7.2 Hz, 5H).

### Step 2—5-Hydroxy-N,N-bis(4-methoxybenzyl) pentane-1-sulfonamide

A mixture of ethyl ethyl 5-(N,N-bis(4-methoxybenzyl)sulfamoyl)pentanoate (12.0 g, 26.7 mmol) and LiBH₄ (1.91 g, 87.7 mmol) in THF (300 mL) was degassed and purged with N₂ three times at 0° C., and then the mixture was stirred at 20° C., for 25 h under N₂ atmosphere. On completion, the reaction mixture was quenched with NH₄Cl (200 mL), then extracted with EtOAc (200 mL×3). The combined organic layers were washed with brine (200 mL×3), dried over Na₂SO₄, filtered and concentrated under reduced pressure to give a residue. The residue was purified by column chromatography (SiO₂, Petroleum ether/Ethyl acetate=3/1 to 1/1) to give the title compound (1.70 g, 14% yield) as a white oil. ¹H NMR (400 MHz, DMSO-d₆) δ=7.18 (d, J=8.4 Hz, 4H), 6.89 (d, J=8.4 Hz, 4H), 4.37 (br s, 1H), 4.20 (s, 4H), 3.74 (s, 6H), 3.37 (br t, J=6.0 Hz, 2H), 3.02-2.97 (m, 2H), 1.66-1.58 (m, 2H), 1.40-1.31 (m, 4H).

### Step 3—5-(N,N-bis(4-methoxybenzyl)sulfamoyl)pentyl 4-methylbenzenesulfonate

To a mixture of 5-hydroxy-N,N-bis(4-methoxybenzyl) pentane-1-sulfonamide (1.65 g, 4.05 mmol), TEA (1.64 g, 16.2 mmol) and DMAP (98.9 mg, 809 umol) in DCM (33 mL) was added 4-methylbenzenesulfonyl chloride (1.16 g, 6.07 mmol) at 0° C. The mixture was stirred at 20° C., for 2 h. On completion, the reaction mixture was quenched with H₂O (20 mL) and then extracted with DCM (20 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=3/1 to 2/1) to give the title compound (1.50 g, 59% yield) as a white solid. ¹H NMR (400 MHZ, DMSO-d₆) δ=7.79 (d, J=8.4 Hz, 2H), 7.48 (d, J=8.0 Hz, 2H), 7.17 (d, J=8.8 Hz, 4H), 6.88 (d, J=8.8 Hz, 4H), 4.18 (s, 4H), 4.00-3.97 (m, 2H), 3.73 (s, 6H), 2.98-2.93 (m, 2H), 2.41 (s, 3H), 1.57-1.50 (m, 4H), 1.30 (br d, J=7.2 Hz, 2H).

### (2S,4R)-4-((tert-butyldimethylsilyl)oxy)-1-((S)-2-(1-fluorocyclopropanecarboxamido)-3,3-dimethylbutanoyl)-N-(4-(4-methylthiazol-5-yl)-2-((5-sulfamoylpentyl)oxy)benzyl)pyrrolidine-2-carboxamide (Intermediate DI)

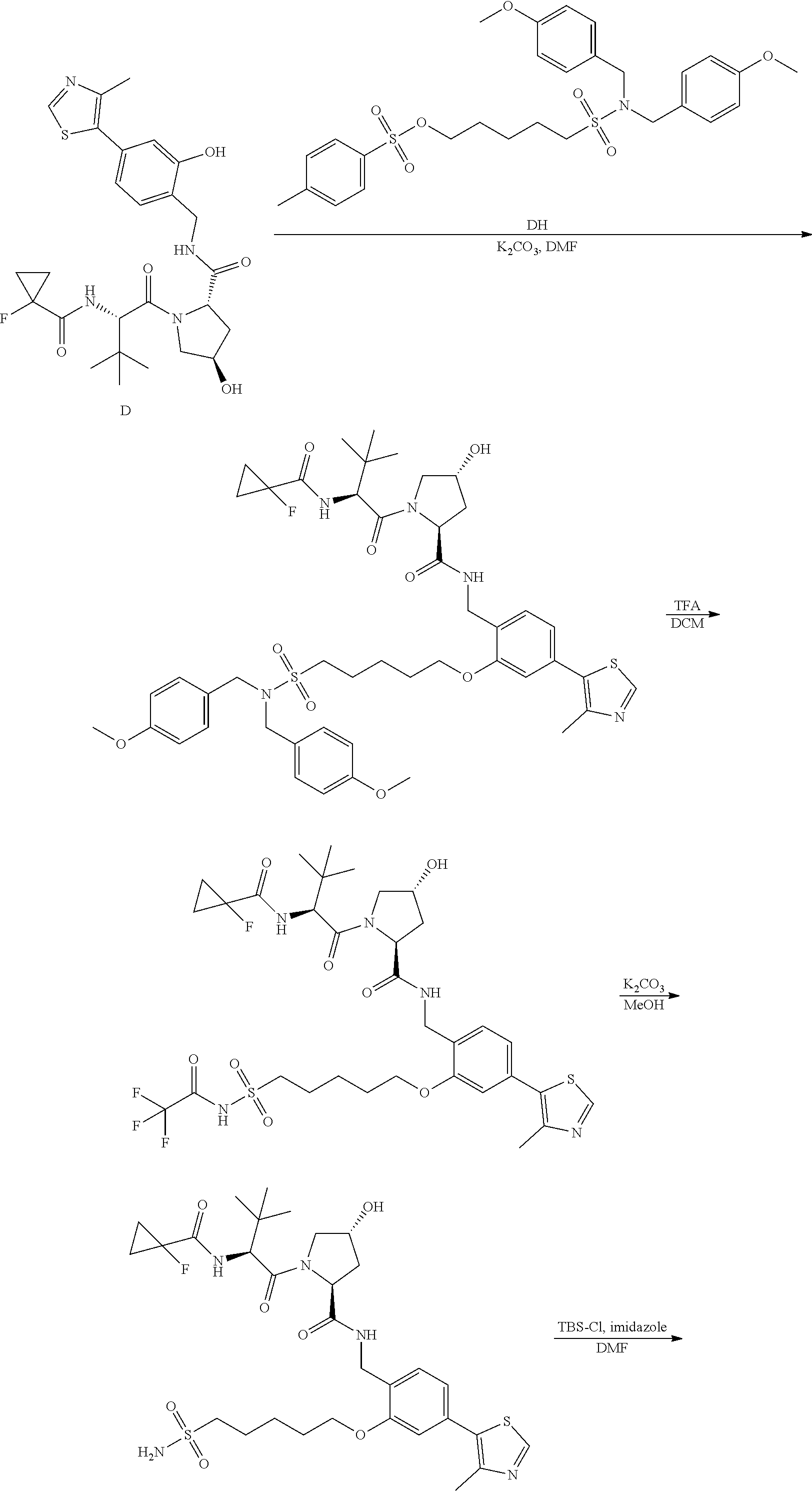

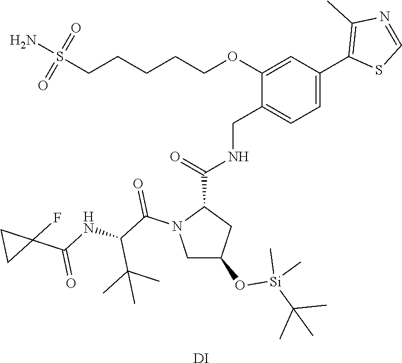 [SMILES: Cc1ncsc1-c1ccc(CNC(=O)[C@@H]2C[C@@H](O[Si](C)(C)C(C)(C)C)CN2C(=O)[C@@H](NC(=O)C2(F)CC2)C(C)(C)C)c(OCCCCCS(N)(=O)=O)c1 ; "DI"]

### Step 1—(2S,4R)—N-(2-((5-(N,N-bis(4-methoxybenzyl)sulfamoyl)pentyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

A mixture of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (474 mg. 890 umol, Intermediate D), 5-(N,N-bis(4-methoxybenzyl)sulfamoyl)pentyl 4-methylbenzenesulfonate (500 mg, 890 umol, Intermediate DH) and K₂CO₃ (615 mg, 4.45 mmol) in DMF (20 mL) was degassed and purged with N₂ three times, then the mixture was stirred at 90° C., for 2 h under N₂ atmosphere. On completion, the reaction mixture was quenched with H₂O (20 mL) and extracted with EtOAc (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3/1 to 1/1) to give the title compound (670 mg. 74% yield) as a white solid. ¹H NMR (400 MHZ, DMSO-d₆) δ=8.99 (s, 1H), 8.50 (br t, J=6.0 Hz, 1H), 7.42 (d, J=7.6 Hz, 1H), 7.29 (dd, J=2.4, 9.2 Hz, 1H), 7.18 (d, J=8.8 Hz, 4H), 7.01 (s, 1H), 6.98-6.95 (m, 1H), 6.88 (d, J=8.8 Hz, 4H), 5.76 (s, 2H), 5.17 (d, J=3.6 Hz, 1H), 4.60 (d, J=9.2 Hz, 1H), 4.53 (t, J=8.0 Hz, 1H), 4.35 (br s, 1H), 4.30 (br d, J=6.0 Hz, 1H), 4.21 (s, 4H), 4.06-4.01 (m, 3H), 3.73 (s, 6H), 3.65 (br d, J=3.6 Hz, 1H), 3.62 (br s, 1H), 3.09-3.05 (m, 2H), 2.47 (s, 3H), 2.12-2.06 (m, 1H), 1.92 (ddd, J=4.4, 8.8, 12.8 Hz, 1H), 1.77-1.67 (m, 5H), 1.55-1.50 (m, 2H), 1.41-1.38 (m, 1H), 1.35 (br d, J=9.6 Hz, 1H), 1.23 (br dd, J=3.2, 8.4 Hz, 2H), 0.96 (s, 9H).

### Step 2—2,2,2-Trifluoroacetic 5-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy) pentane-1-sulfonic anhydride

A mixture of (2S,4R)—N-(2-((5-(N,N-bis(4-methoxybenzyl)sulfamoyl)pentyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (670 mg. 727 umol) and TFA (3.08 g, 27.0 mmol) in DCM (4 mL) the mixture was stirred at 25° C., for 12 h. On completion, the reaction mixture was quenched with H₂O (2 mL) and then extracted with DCM (2 mL×3). The combined organic layers were washed with brine (2 mL×3), dried over Na₂SO₄, filtered and concentrated under reduced pressure to give a residue. The residue was title compound (500 mg) as a white solid. LC-MS (ESI⁺) m/z 778.3 (M/2).

### Step 3—(2S,4R)-1-((S)-2-(1-fluorocyclopropanecarboxamido)-3,3-dimethylbutanoyl)-4-hydroxy-N-(4-(4-methylthiazol-5-yl)-2-((5-sulfamoylpentyl)oxy)benzyl)pyrrolidine-2-carboxamide

A mixture of (2S,4R)-1-((S)-2-(1-fluorocyclopropanecarboxamido)-3,3-dimethylbutanoyl)-4-hydroxy-N-(4-(4-methylthiazol-5-yl)-2-((5-(N-(2,2,2-trifluoroacetyl)sulfamoyl)pentyl)oxy)benzyl)pyrrolidine-2-carboxamide (500 mg. 643 umol) and K₂CO₃ (444 mg. 3.21 mmol) in MeOH (5 mL) was stirred at 25° C., for 2 h. On completion, the reaction mixture was quenched with H₂O (5 mL) and then extracted with DCM (5 mL×3). The combined organic layers were washed with brine (3 mL×3), dried over Na₂SO₄, filtered and concentrated under reduced pressure to give a residue. The residue was purified by column chromatography (SiO₂, Petroleum ether/Ethyl acetate=1/1 to 1/5) to give the title compound (400 mg, 82% yield) as a white solid. LC-MS (ESI⁺) m/z 682.4 (M/2)⁺.

### Step 4 (2S,4R)-4-((tert-butyldimethylsilyl)oxy)-1-((S)-2-(1-fluorocyclopropanecarboxamido)-3,3-dimethylbutanoyl)-N-(4-(4-methylthiazol-5-yl)-2-((5-sulfamoylpentyl)oxy)benzyl)pyrrolidine-2-carboxamide

A mixture of (2S,4R)-1-((S)-2-(1-fluorocyclopropanecarboxamido)-3,3-dimethylbutanoyl)-4-hydroxy-N-(4-(4-methylthiazol-5-yl)-2-((5-sulfamoylpentyl)oxy)benzyl)pyrrolidine-2-carboxamide (400 mg, 352 umol) in DMF (4 mL) was added imidazole (312 mg, 4.58 mmol) and tert-butyl-chloro-dimethyl-silane (531 mg, 3.52 mmol). The mixture was stirred at 20° C., for 12 h. On completion, the reaction mixture was quenched with H₂O (10 mL) and then extracted with EtOAc (10 mL×3). The combined organic layers were washed with brine (4 mL×3), dried over Na₂SO₄, filtered and concentrated under reduced pressure to give a residue. The residue was purified by column chromatography (SiO₂, Petroleum ether/Ethyl acetate=1/1 to 1/2) to give the title compound (270 mg, 87% yield) as a white oil. ¹H NMR (400 MHZ, DMSO-d₆) δ=8.91 (s, 1H), 8.45 (br t, J=6.0 Hz, 1H), 7.33 (d, J=8.0 Hz, 1H), 7.21 (dd, J=2.4, 9.2 Hz, 1H), 6.94-6.92 (m, 1H), 6.87 (dd, J=1.2, 7.6 Hz, 1H), 6.66 (s, 2H), 4.55 (d, J=9.2 Hz, 1H), 4.46 (br d, J=8.0 Hz, 2H), 4.27-4.21 (m, 1H), 4.16-4.10 (m, 1H), 4.00-3.97 (m, 2H), 2.95-2.91 (m, 2H), 2.39 (s, 3H), 2.05-1.99 (m, 1H), 1.91-1.85 (m, 1H), 1.75-1.66 (m, 5H), 1.53-1.48 (m, 2H), 1.31-1.24 (m, 2H), 1.18-1.11 (m, 2H), 1.09-0.99 (m, 2H), 0.89 (s, 9H), 0.74 (s, 9H), 0.00 (s, 3H), −0.02 (s, 3H).

### 8-(N,N-bis(4-methoxybenzyl)sulfamoyl) octyl 4-methylbenzenesulfonate (Intermediate DJ)

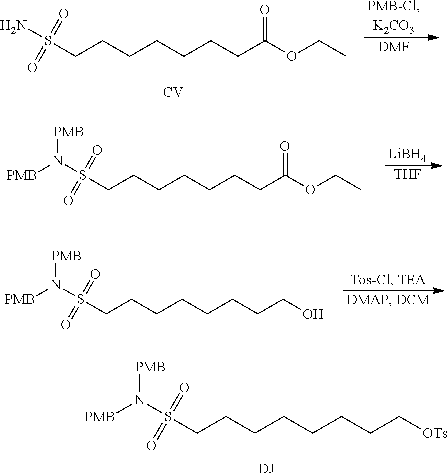

### Step 1—Ethyl 8-(N, N-bis(4-methoxybenzyl)sulfamoyl) octanoate

To a solution of ethyl 8-sulfamoyloctanoate (500 mg, 1.59 mmol, Intermediate CV) in DMF (5 mL) was added K₂CO₃ (880 mg, 6.37 mmol), and PMB-Cl (623 mg, 3.98 mmol, 542 uL) at 0° C., then the mixture was stirred 50° C., for 16 h. On completion, the reaction mixture was quenched with NH₄Cl (3 mL) and extracted with EtOAc (5 mL×2). The combined organic layers were dried over Na₂SO₄ and evaporated. The residue was purified by silica gel column chromatography (Petroleum ether:Ethyl acetate=20:1 to 10:1) to give the title compound (490 mg, 63% yield) as a white solid. ¹H NMR (400 MHZ, DMSO-d₆) ¿ 7.18 (br d, J=8.4 Hz, 4H), 6.89 (br d, J=8.4 Hz, 4H), 4.19 (s, 4H), 4.04 (q, J=7.2 Hz, 2H), 3.73 (s, 6H), 3.03-2.92 (m, 2H), 2.27 (br t, J=7.2 Hz, 2H), 1.62-1.54 (m, 2H), 1.49 (br d, J=6.4 Hz, 2H), 1.27 (br d, J=6.8 Hz, 2H), 1.21 (br d, J=8.0 Hz, 4H), 1.19-1.15 (m, 3H).

### Step 2—8-Hydroxy-N,N-bis(4-methoxybenzyl) octane-1-sulfonamide

To a solution of ethyl 8-(N,N-bis(4-methoxybenzyl)sulfamoyl) octanoate (490 mg, 997 umol) in THF (5 mL) was added LiBH₄ (109 mg, 4.98 mmol) at 0° C., under N₂, then the mixture was stirred at 25° C., for 12 h. On completion, the reaction mixture was quenched with NH₄Cl (2 mL) and extracted by EtOAc (3 mL×3). The combined organic layers were washed with brine (3 mL×3), dried over Na₂SO₄ and evaporated to give the title compound (420 mg) as a white solid. ¹H NMR (400 MHZ, DMSO-d₆) δ 7.21-7.14 (m, 4H), 6.91-6.85 (m, 4H), 4.25-4.14 (m, 4H), 3.74-3.72 (m, 6H), 3.70-3.63 (m, 1H), 3.41-3.33 (m, 2H), 3.01-2.92 (m, 2H), 1.60-1.53 (m, 2H), 1.47-1.37 (m, 2H), 1.28-1.18 (m, 8H).

### Step 3—8-(N, N-bis(4-methoxybenzyl)sulfamoyl) octyl 4-methylbenzenesulfonate

To a solution of 8-hydroxy-N,N-bis(4-methoxybenzyl) octane-1-sulfonamide (135 mg, 300 umol) and TEA (122 mg, 1.20 mmol, 167 uL), and DMAP (7.34 mg, 60.1 umol) in DCM (4 mL) was added TosCl (85.9 mg, 450 umol) at 0° C., then the mixture was stirred at 20° C., for 5 h. On completion, the reaction mixture was quenched with NH₄Cl (3 mL) and extracted by EtOAc (5 mL×3). The combined organic layers were dried over Na₂SO₄ and evaporated. The residue was purified by column chromatography (SiO₂, Petroleum ether/Ethyl acetate=5:1 to 3:1) to give the title compound (145 mg, 80% yield) as a white solid. ¹H NMR (400 MHZ, DMSO-d₆) δ 7.78 (br d, J=8.0 Hz, 2H), 7.48 (br d, J=8.0 Hz, 2H), 7.18 (br d, J=8.0 Hz, 4H), 6.88 (br d, J=8.0 Hz, 4H), 4.19 (s, 4H), 4.00 (br t, J=6.0 Hz, 2H), 3.73 (s, 6H), 2.99-2.92 (m, 2H), 2.41 (s, 3H), 1.54 (br s, 4H), 1.26-1.15 (m, 5H), 1.11 (br s, 4H).

### (2S,4R)-4-((tert-butyldimethylsilyl)oxy)-1-((S)-2-(1-fluorocyclopropanecarboxamido)-3,3-dimethylbutanoyl)-N-(4-(4-methylthiazol-5-yl)-2-((8-sulfamoyloctyl)oxy)benzyl)pyrrolidine-2-carboxamide (Intermediate DK)

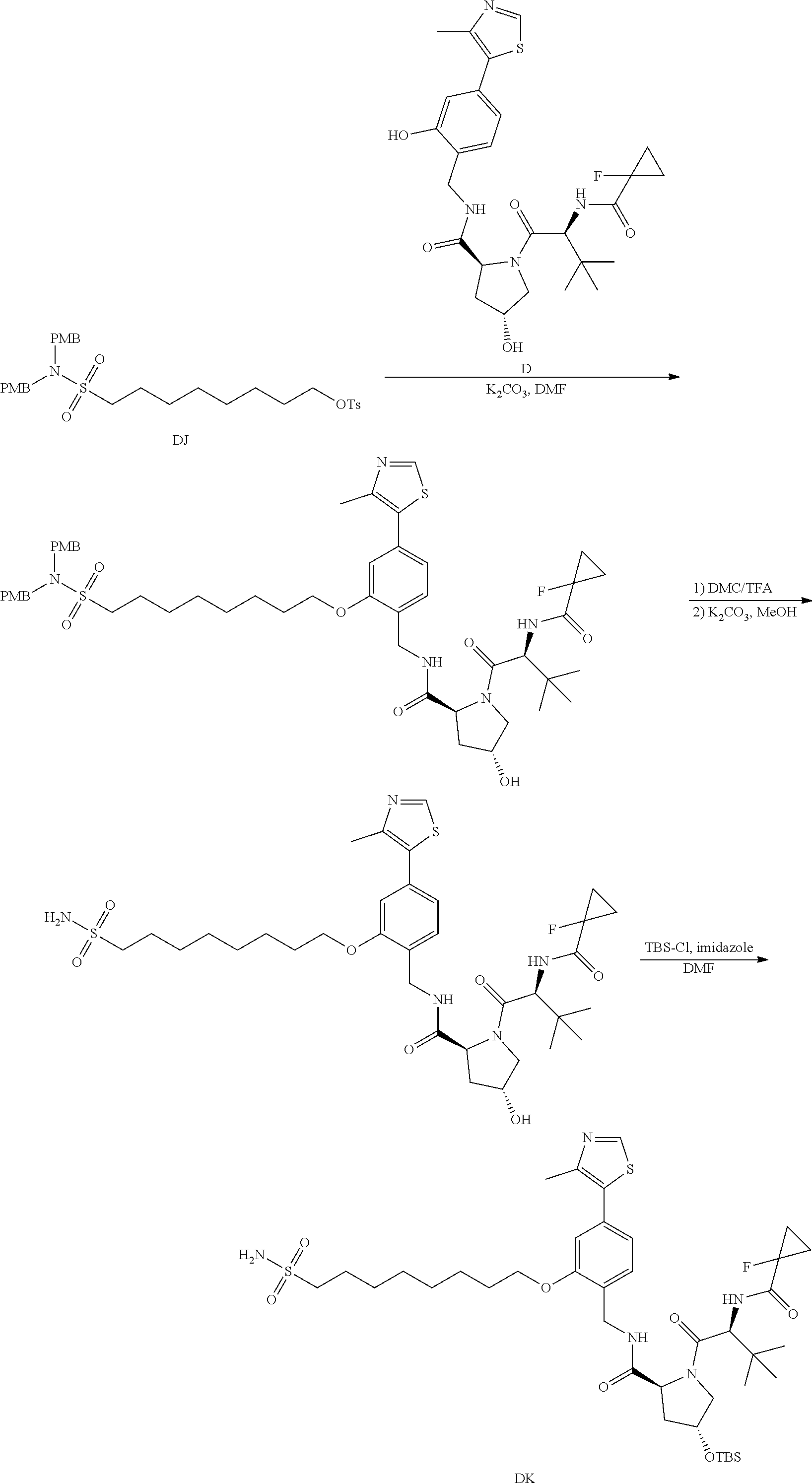

### Step 1—(2S,4R)—N-(2-((8-(N,N-bis(4-methoxybenzyl)sulfamoyl) octyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of 8-(N,N-bis(4-methoxybenzyl)sulfamoyl) octyl 4-methylbenzenesulfonate (145 mg, 240 umol, Intermediate DJ), (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (128 mg, 240 umol, Intermediate D) in DMF (2 mL) was added K₂CO; (166 mg, 1.2 mmol), then the mixture was stirred at 90° C., for 2 h. On completion, the reaction mixture was quenched with NH₄Cl (3 mL) and extracted with EtOAc (5 mL×3). The combined organic layers were dried over Na₂SO₄ and evaporated. The residue was purified by column chromatography (SiO₂, Petroleum ether/Ethyl acetate=7:1˜ 3:1) to afford the title compound (175 mg, 76% yield) as a white solid. LC-MS (ESI) m/z 964.4 (M+H).

### Step 2—(2S,4R)-1-((S)-2-(1-fluorocyclopropanecarboxamido)-3,3-dimethylbutanoyl)-4-hydroxy-N-(4-(4-methylthiazol-5-yl)-2-((8-sulfamoyloctyl)oxy)benzyl)pyrrolidine-2-carboxamide

A solution of (2S,4R)—N-(2-((8-(N,N-bis(4-methoxy benzyl)sulfamoyl) octyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (175 mg, 182 umol) in TFA (0.5 mL) and DCM (1 mL) was stirred at 25° C., for 12 h. On completion, the solution was concentrated in vacuo to give a mixture. The mixture was dissolved in MeOH (1.5 mL) and then, K₂CO; (95.5 mg, 691 umol) was added and the mixture was stirred at 25° C., for 3 h. On completion, the reaction mixture was quenched with NH₄Cl (3 mL) and extracted with EtOAc (5 mL×3). The combined organic layers were dried over Na₂SO₄ and evaporated. The residue was purified by column chromatography (SiO₂, Petroleum ether/Ethyl acetate=5:1 to 1:1) to afford the title compound (135 mg) as a white solid. LC-MS (ESI) m/z 724.4 (M+H).

### Step 3 (2S,4R)-4-((tert-butyldimethylsilyl)oxy)-1-((S)-2-(1-fluorocyclopropanecarboxamido)-3,3-dimethylbutanoyl)-N-(4-(4-methylthiazol-5-yl)-2-((8-sulfamoyloctyl)oxy)benzyl)pyrrolidine-2-carboxamide

To a solution of (2S,4R)-1-((S)-2-(1-fluorocyclopropanecarboxamido)-3,3-dimethylbutanoyl)-4-hydroxy-N-(4-(4-methylthiazol-5-yl)-2-((8-sulfamoyloctyl)oxy)benzyl)pyrrolidine-2-carboxamide (132 mg, 182 umol) and TBSCl (275 mg, 1.82 mmol, 223 uL) in DMF (2 mL) was added imidazole (161 mg, 2.37 mmol), then the mixture was stirred at 25° C., for 17 h. On completion, the reaction mixture was quenched with NH₄Cl (2 mL) and extracted with EtOAc (5 mL×3). The combined organic layers were washed with brine (2 mL×3), dried over Na₂SO₄ and evaporated. The residue was purified by column chromatography (SiO₂, Petroleum ether/Ethyl acetate=5:1 to 1:1) to afford the title compound (115 mg, 75% yield) as a white solid. LC-MS (ESI) m/z 838.9 (M+H).

### Methyl 3-(4-sulfamoylphenyl)propanoate (Intermediate DL)

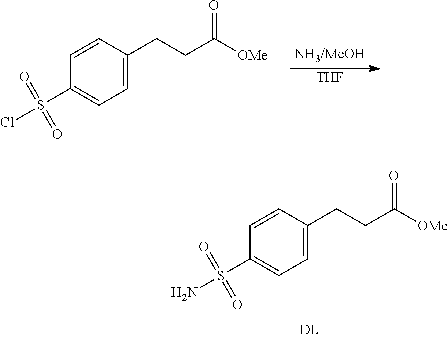 [SMILES: COC(=O)CCc1ccc(S(=O)(=O)Cl)cc1 ; COC(=O)CCc1ccc(S(N)(=O)=O)cc1 ; "THF"]

To a solution of methyl 3-(4-chlorosulfonylphenyl)propanoate (5 g, 20 mmol, CAS #374537-95-8) in THF (50 mL) was added NH₃/MeOH (7 M, 13.6 mL). The mixture was stirred at 25° C., for 12 h. On completion, the solvent was removed under reduced pressure and the residue was dissolved in DCM. The organic phase is washed with a saturated aqueous solution of NH₄Cl, then brine. The organic phase was then concentrated under reduced pressure, then dried under vacuum at 40° C., to give the title compound (4 g, 86% yield) as a white solid. LC-MS (ESI⁺) m/z 487.1 (M+H)⁺.

### 3-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)propanoic acid (Intermediate DM)

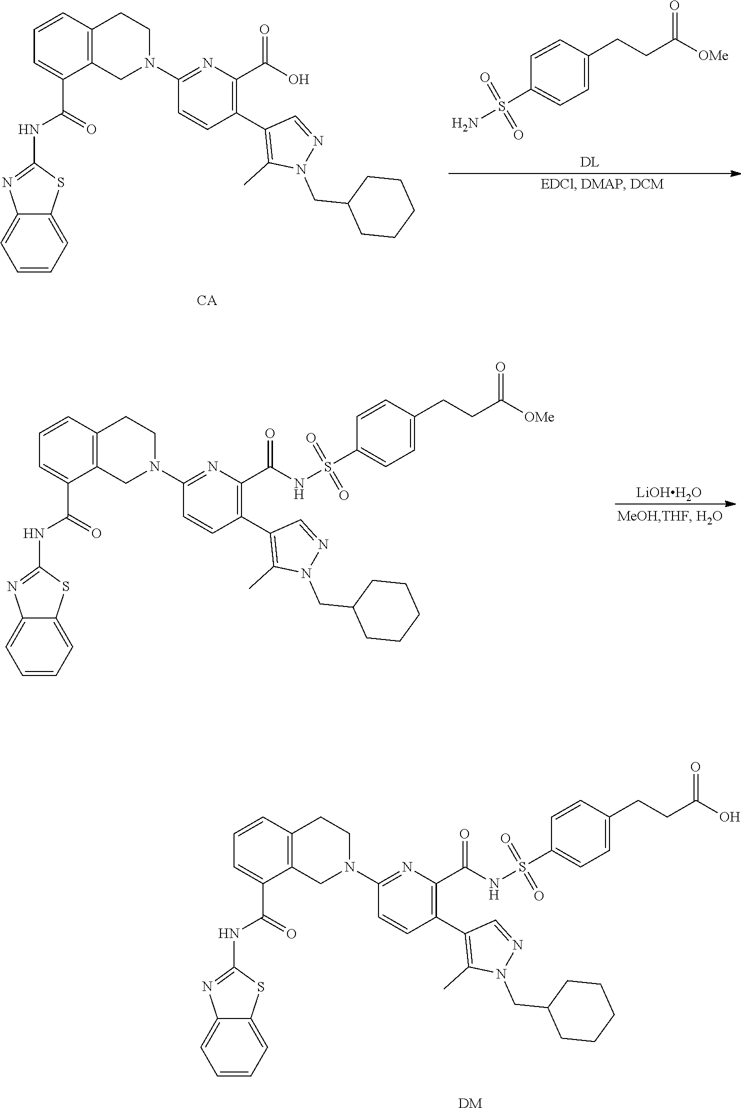

### Step 1—Methyl 3-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)propanoate

To a solution of methyl 3-(4-sulfamoylphenyl)propanoate (104 mg, 428 umol, Intermediate DL), 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (130 mg, 214 umol, Intermediate CA) in DCM (3 mL) was added EDCI (205 mg, 1.07 mmol) and DMAP (131 mg, 1.07 mmol). The mixture was stirred at 25° C. for 12 h. On completion, the mixture was filtered, the filtrate was dissolved with H₂O (2 mL×2), and extracted with EtOAc (2 mL×2). Then the organic layer was washed with brine (2 mL), dried over anhydrous Na₂SO₄, filtered and concentrated under reduced pressure to give a residue. The residue was purified by column chromatography (SiO₂, Petroleum ether:Ethyl acetate=5:1 to Dichloromethane; Methanol=10:1) to give the title compound (170 mg, 95% yield) as a white solid. LC-MS (ESI⁺) m/z 832.3 (M+H)⁺.

### Step 2—3-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)propanoic acid

To a solution of methyl 3-[4-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]phenyl]propanoate (170 mg, 204 umol) in MeOH (1 mL), THF (1 mL), and H₂O (1 mL) was added LiOH·H₂O (42.9 mg, 1.02 mmol). The mixture was stirred at 25° C., for 2 h. On completion, the mixture was adjusted pH to acidity and concentrated in vacuo. The mixture was diluted with H₂O (3 mL), and extracted with EtOAc mL (2 mL×3). The organic phase was then washed with brine (3 mL), dried over anhydrous Na₂SO₄, filtered and concentrated under reduced pressure to give the title compound (160 mg, 96% yield) as a white solid. LC-MS (ESI⁺) m/z 818.3 (M+H)⁺.

### 3-(4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)phenyl)propanoic acid (Intermediate DN)

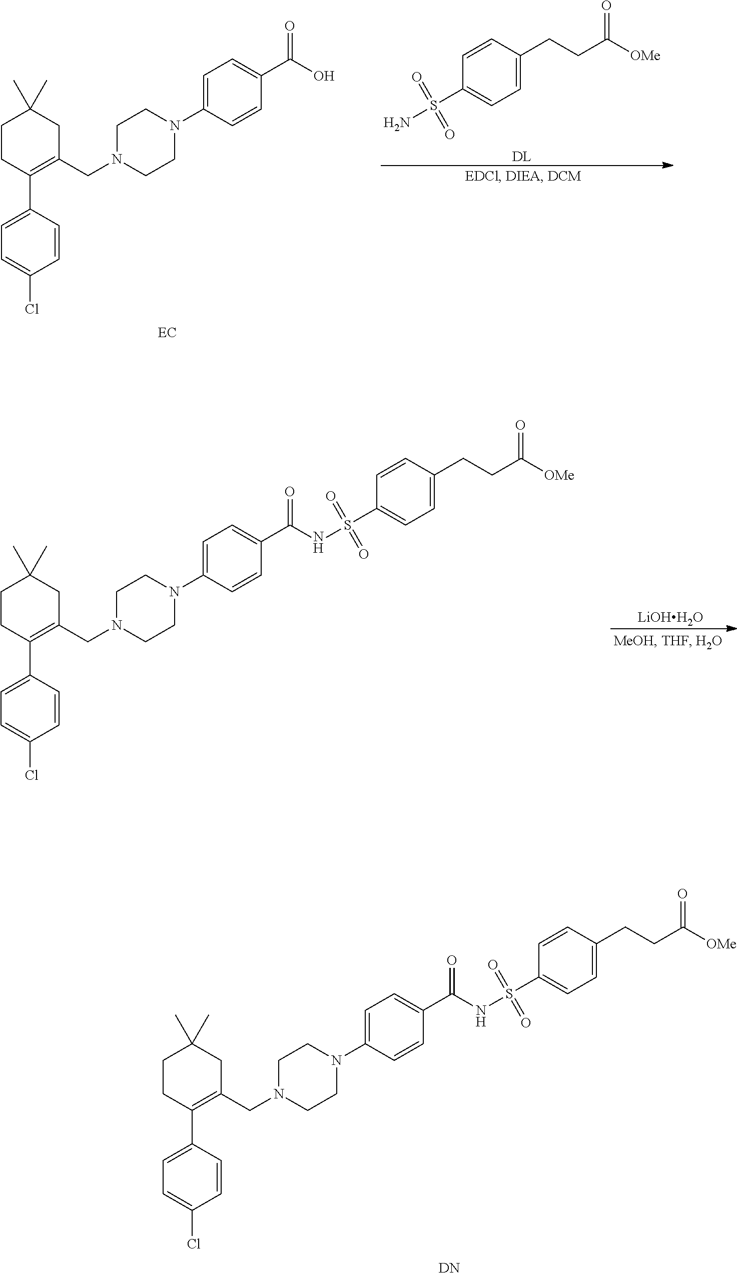

### Step 1—Methyl 3-(4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)phenyl)propanoate

To a solution of 4-[4-[[2-(4-chlorophenyl)-5,5-dimethyl-cyclohexen-1-yl]methyl]piperazin-1-yl]benzoic acid (200 mg, 455 umol, Intermediate EC), methyl 3-(4-sulfamoylphenyl)propanoate (221 mg, 911 umol, Intermediate DL) in DCM (2 mL) was added EDCI (436 mg, 2.28 mmol) and DMAP (278 mg, 2.28 mmol). The mixture was stirred at 25° C., for 12 h. On completion, the mixture was filtered and the filtrate was diluted with H₂O (2 mL×2), and extracted with EtOAc (2 mL×2). The combined organic layer was then washed with brine (2 mL), dried over anhydrous Na₂SO₄, filtered and concentrated under reduced pressure to give a residue. The residue was purified by column chromatography (SiO₂, Petroleum ether:Ethyl acetate=5:1 to Petroleum ether:Ethyl acetate=1:1) to give the title compound (280 mg, 93% yield) as a white solid. LC-MS (ESI⁺) m/z 664.3 (M+H).

### Step 2—3-(4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)phenyl)propanoic acid

To a solution of methyl 3-[4-[[4-[4-[[2-(4-chlorophenyl)-5,5-dimethyl-cyclohexen-1-yl]methyl]piperazin-1-yl]benzoyl]sulfamoyl]phenyl]propanoate (280 mg, 422 umol) in MeOH (1.5 mL), THF (1.5 mL), and H₂O (1.5 mL) was added LiOH·H₂O (88.4 mg. 2.11 mmol). The mixture was stirred at 25° C., for 2 hr. On completion, the mixture was adjusted pH to 4˜5 with HCl (1 M) and concentrated in vacuo. The mixture was diluted with H₂O (3 mL), and extracted with EtOAc (2 mL×3). The organic layer was then washed with brine (3 mL), dried over anhydrous Na₂SO₄, filtered and concentrated under reduced pressure to give the title compound (260 mg) as a white solid. LC-MS (ESI⁺) m/z 650.4 (M+H).

### (R)-tert-butyl (4-oxo-1-(phenylthio)butan-2-yl) carbamate (Intermediate DQ)

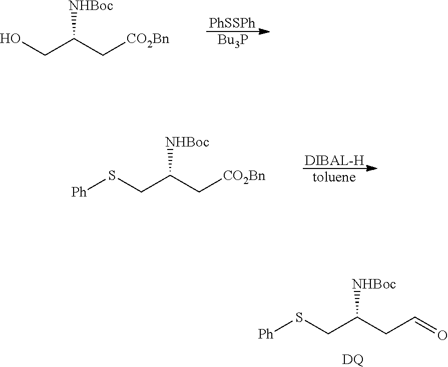

### Step 1—Benzyl (R)-3-((tert-butoxycarbonyl)amino)-4-(phenylthio)butanoate

To a solution of benzyl (R)-3-((tert-butoxycarbonyl)amino)-4-hydroxybutanoate (50.0 g, 161 mmol), (phenyldisulfanyl)benzene (45.5 g, 208 mmol) and tributylphosphane (42.5 g, 210 mmol, 51.8 mL) in toluene (500 mL) was heated at 80° C., for 12 h under N₂. On completion, the reaction mixture was quenched with IM NaOH (500 mL) at 20° C., and then extracted with diluted with EtOAc (400 mL×3). The combined organic layers were washed with brine (800 mL), dried over Na₂SO₄, filtered and concentrated under reduced pressure to give a residue. The residue was purified by column chromatography (SiO₂. Petroleum ether/Ethyl acetate=50/1:10/1, Rf=0.32) to give the title compound (35.0 g, 53% yield) as a light yellow oil. LC-MS (ESI) m/z 302.2 (M+H).

### Step 2—Tert-butyl (R)-(4-oxo-1-(phenylthio)butan-2-yl) carbamate

A solution of DIBAL-H (1.00 M, 143 mL) was added dropwise to a solution of benzyl (R)-3-((tert-butoxycarbonyl)amino)-4-(phenylthio)butanoate (25.0 g, 62.3 mmol) in toluene (250 mL) at −78° C., and stirred for 3 h. On completion, the reaction mixture was quenched with saturated NH₄Cl (400 mL) at −78° C., then warmed to room temperature, and extracted with diluted with EtOAc (200 mL×3). The combined organic layers were washed with brine (400 mL), dried over Na₂SO₄, filtered and concentrated under reduced pressure to give the title compound (22.5 g) as a yellow oil. LC-MS (ESI⁺) m/z 240.1 (M+H)⁺.

### (R)-2,2,2-trichloroethyl 4-(3-amino-4-(phenylthio)butyl)piperazine-1-carboxylate (Intermediate DR)

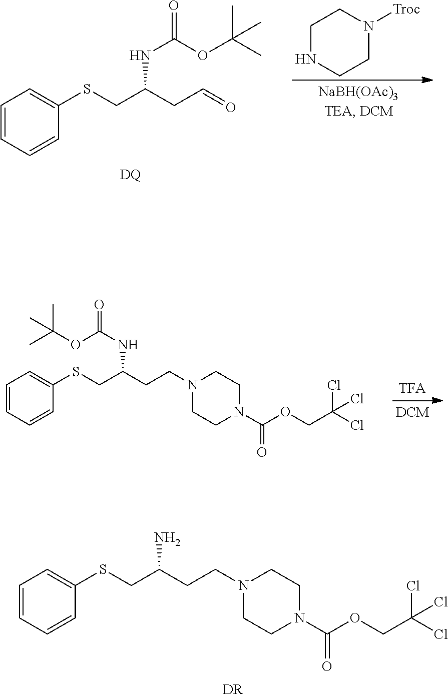

### Step 1—(R)-2,2,2-trichloroethyl 4-(3-((tert-butoxycarbonyl)amino)-4-(phenylthio)butyl)piperazine-1-carboxylate

A mixture of 2,2,2-trichloroethyl piperazine-1-carboxylate (2.97 g, 11.4 mmol, CAS #779985-91-0), (R)-tert-butyl (4-oxo-1-(phenylthio)butan-2-yl) carbamate (2.40 g, 8.12 mmol, Intermediate DQ) and TEA (3.29 g, 32.5 mmol) in DCM (24 mL) was added NaBH (OAc); (2.58 g, 12.2 mmol). Then the mixture was stirred at 20° C., for 12 h under N₂ atmosphere. On completion, the reaction mixture was quenched with H₂O (20 mL), and then extracted with DCM (20 mL×3). The combined organic layers were washed with brine (10 mL×3), dried over Na₂SO₄, filtered and concentrated under reduced pressure to give a residue. The residue was purified by prep-HPLC (water (FA)-ACN]: B %: 55%-85%) to give the title compound (3.80 g, 78% yield) as a light yellow oil. ¹H NMR (400 MHZ, DMSO-d₆) ô=8.17 (s, 1H), 7.38-7.33 (m, 2H), 7.33-7.28 (m, 2H), 7.20-7.15 (m, 1H), 6.87 (br d, J=8.4 Hz, 1H), 4.85 (s, 2H), 3.62 (br d, J=5.2 Hz, 1H), 3.43 (br s, 4H), 3.02 (br d, J=6.4 Hz, 2H), 2.33 (br d, J=1.6 Hz, 6H), 1.80-1.71 (m, 1H), 1.57 (td, J=6.4, 13.2 Hz, 1H), 1.38 (s, 9H).

### Step 2—(R)-2,2,2-trichloroethyl 4-(3-amino-4-(phenylthio)butyl)piperazine-1-carboxylate

A mixture of (R)-2,2,2-trichloroethyl 4-(3-((tert-butoxycarbonyl)amino)-4-(phenylthio)butyl)piperazine-1-carboxylate (3.80 g, 5.62 mmol) and TFA (14.6 g, 128 mmol) in DCM (38 mL) was stirred at 20° C., for 12 h under N₂ atmosphere. On completion, the reaction mixture was quenched with NaHCO; (50 mL) and then extracted with DCM (50 mL×3). The combined organic layers were washed with brine (20 mL×3), dried over Na₂SO₄, filtered and concentrated under reduced pressure to give the title compound (3.00 g) as a brown oil. ¹H NMR (400 MHZ, DMSO-d₆) δ=7.39-7.35 (m, 2H), 7.34-7.29 (m, 2H), 7.20 (d, J=7.2 Hz, 1H), 5.75 (s, 1H), 4.84 (s, 2H), 3.43 (br s, 4H), 3.13-3.06 (m, 1H), 3.00-2.93 (m, 2H), 2.44-2.37 (m, 2H), 2.34 (br t, J=4.8 Hz, 4H), 1.74 (dt, J=4.4, 6.8 Hz, 1H), 1.55-1.47 (m, 1H).

### (R)-2,2,2-trichloroethyl 4-(4-(phenylthio)-3-((4-sulfamoyl-2-((trifluoromethyl)sulfonyl)phenyl)amino)butyl)piperazine-1-carboxylate (Intermediate DS)

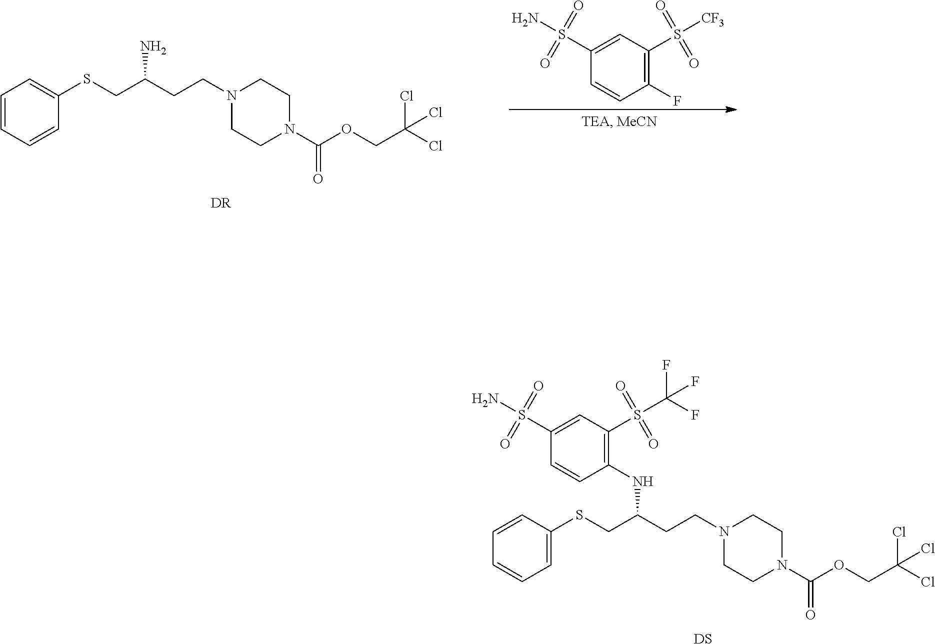 [SMILES: N[C@H](CCN1CCN(C(=O)OCC(Cl)(Cl)Cl)CC1)CSc1ccccc1 ; NS(=O)(=O)c1ccc(F)c(S(=O)(=O)C(F)(F)F)c1 ; "DR" ; NS(=O)(=O)c1ccc(N[C@H](CCN2CCN(C(=O)OCC(Cl)(Cl)Cl)CC2)CSc2ccccc2)c(S(=O)(=O)C(F)(F)F)c1 ; "DS"]

A mixture of (R)-2,2,2-trichloroethyl 4-(3-amino-4-(phenylthio)butyl)piperazine-1-(3.00 g, carboxylate 6.81 mmol, Intermediate DR) and 4-fluoro-3-(trifluoromethylsulfonyl)benzenesulfonamide (2.09 g, 6.81 mmol, CAS #1027345-08-9) in ACN (21 mL) was added to TEA (3.44 g, 34.0 mmol), then the reaction mixture was stirred at 20° C., for 12 h. On completion, the reaction mixture was quenched with H₂O (30 mL) and then extracted with EtOAc (30 mL×3). The combined organic layers were washed with brine (20 mL×3), dried over Na₂SO₄, filtered and concentrated under reduced pressure to give a residue. The residue was purified by reversed-phase HPLC (ACN: 0.1% NH₃·H₂O=50% to 80%) to give the title compound (4.20 g, 76% yield) as a white solid. ¹H NMR (400 MHZ, DMSO-d₆) ¿=7.98 (d, J=2.0 Hz, 1H), 7.87 (dd, J=2.0, 9.2 Hz, 1H), 7.39-7.28 (m, 6H), 7.23-7.19 (m, 1H), 7.08 (d, J=9.6 Hz, 1H), 6.92 (br d, J=9.2 Hz, 1H), 4.84 (s, 2H), 4.11 (br d, J=4.0 Hz, 1H), 3.45-3.37 (m, 3H), 3.36 (br s, 1H), 3.31-3.27 (m, 1H), 2.41-2.26 (m, 5H), 2.23-2.17 (m, 2H), 2.00-1.91 (m, 1H), 1.80-1.71 (m, 1H).

### Methyl 2-(5-bromo-6-(tert-butoxycarbonyl)pyridin-2-yl)-6-((2-((tert-butoxycarbonyl)(methyl)amino)ethyl)(methyl)amino)-1,2,3,4-tetrahydroisoquinoline-8-carboxylate (Intermediate DT)

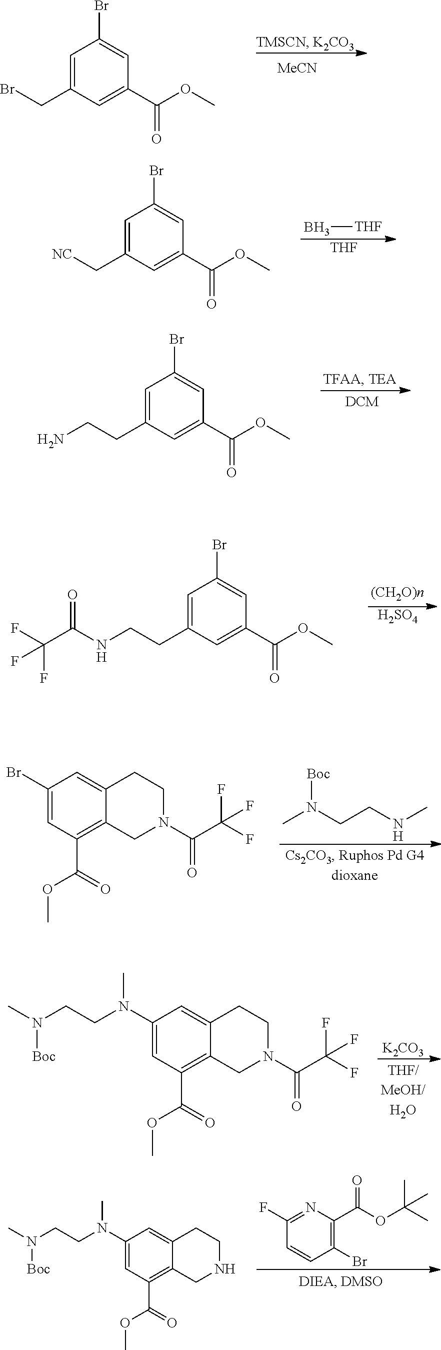

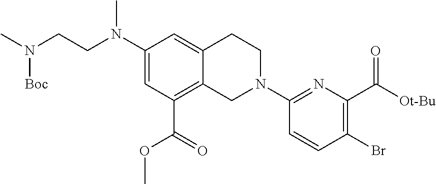 [SMILES: COC(=O)c1cc(N(C)CCN(C)C(=O)OC(C)(C)C)cc2c1CN(c1ccc(Br)c(C(=O)OC(C)(C)C)n1)CC2]

### Step 1—Methyl 3-bromo-5-(cyanomethyl)benzoate

To a solution of methyl 3-bromo-5-(bromomethyl)benzoate (12.5 g, 40.6 mmol, CAS #877624-40-3) and trimethylsilylformonitrile (6.04 g, 60.8 mmol) in ACN (500 mL) was added dipotassium carbonate (8.41 g, 60.8 mmol) at 20° C., under nitrogen flow. Then the reaction was stirred at 90° C., for 10 h under nitrogen atmosphere. On completion, the reaction was filtered to get the filtrate and concentrated to give a residue. The residue was purified by column chromatography on silica gel (eluted with petroleum ether:ethyl acetate=100:1 to 100:15) to give the title compound (10 g, 97% yield) as a yellow solid. 1H NMR (400 MHZ, CDCl₃) δ=8.16 (s, 1H), 7.96 (s, 1H), 7.72 (s, 1H), 3.96 (s, 3H), 3.81 (s, 2H). Step 2—Methyl 3-(2-aminoethyl)-5-bromobenzoate

To a solution of methyl 3-bromo-5-(cyanomethyl)benzoate (10 g, 39.3 mmol) in THF (125 mL) was added BH₃·THF (1 M, 78.72 mL) dropwise slowly at 0° C., under nitrogen flow. Then the reaction was stirred at 20° C., for 10 h under nitrogen atmosphere. On completion, the reaction was carefully quenched with methanol (32 mL), and then concentrated to 32 mL volume. The mixture was then taken up in 100 mL methanol/100 mL 4M HCl/dioxane, and stirred overnight. The organics were removed by evaporation under reduced pressure to obtained a residue. The residue was dissolved in EtOAc (150 mL) and poured into water (150 mL). The aqueous layer was basified with solid K₂CO; to pH=9, then extracted with EtOAc (150 mL×2). The combined organic phase was washed with brine (100 mL×2), and dried over Na₂SO₄. The mixture was then filtered and concentrated to give a residue. The residue was purified by column chromatography on silica gel (eluted with petroleum ether:ethyl acetate=100:1 to 100:30) to give the title compound (5.9 g, 58% yield) as a white solid. ¹H NMR (400 MHZ, CDCl₃) δ=8.02 (s, 1H), 7.82 (s, 1H), 7.56 (s, 1H), 4.01-3.85 (m, 3H), 3.12-3.00 (m, 2H), 2.85 (br s, 2H).

### Step 3—Methyl 3-bromo-5-(2-(2,2,2-trifluoroacetamido)ethyl)benzoate

To a solution of methyl 3-(2-aminoethyl)-5-bromobenzoate (5.9 g, 22.8 mmol) and TEA (3.47 g. 34.3 mmol) in DCM (100 mL) was added TFAA (5.76 g, 27.4 mmol) dropwise slowly at 0° C., under nitrogen flow. Then the reaction was stirred at 20° C., for 10 h under nitrogen atmosphere. On completion, the reaction was poured into water (150 mL) and extracted with DCM (100 mL×2). The combined organic phase is washed with brine (70 mL×2), and dried over Na₂SO₄. Then the mixture was filtered and concentrated to give a residue. The residue was purified by column chromatography on silica gel (eluted with petroleum ether:ethyl acetate=100:1 to 100:20) to give the title compound (5.9 g, 73% yield) as a colorless oil. ¹H NMR (400 MHZ, CDCl₃) δ=8.05 (t, J=1.6 Hz, 1H), 7.80 (s, 1H), 7.54 (t, J=1.6 Hz, 1H), 6.62 (br s, 1H), 3.92 (s, 3H), 3.63 (q, J=6.8 Hz, 2H), 2.93 (t, J=7.2 Hz, 2H).

### Step 4—Methyl 6-bromo-2-(2,2,2-trifluoroacetyl)-1,2,3,4-tetrahydroisoquinoline-8-carboxylate

To a solution of methyl 3-bromo-5-(2-(2,2,2-trifluoroacetamido)ethyl)benzoate (5.9 g, 16.66 mmol) in H₂SO₄ (48 mL) was added formaldehyde (2.50 g, 83.31 mmol) at 0° C., under nitrogen flow. Then the reaction was stirred at 20° C., for 1 h under nitrogen atmosphere. On completion, the reaction was poured into ice water (300 mL) and extracted with EtOAc (100 mL×2). The combined organic phase is washed with brine (70 mL×2), and dried over Na₂SO₄. Then the mixture was filtered and concentrated to give a residue. The residue was purified by column chromatography on silica gel (eluted with petroleum ether: ethyl acetate=100:1 to 100:17) to give the title compound (3.9 g, 64% yield) as a yellow oil. ¹H NMR (400 MHZ, CDCl₃) δ 7.98 (dd, J=2.0, 17.6 Hz, 1H), 7.43 (dd, J=1.4, 15.2 Hz, 1H), 5.06 (d, J=14.4 Hz, 2H), 3.85 (d, J=1.6 Hz, 3H), 3.83-3.74 (m, 2H), 2.93 (q. J=6.4 Hz, 2H).

### Step 5—Methyl 6-((2-((tert-butoxycarbonyl)(methyl)amino)ethyl)(methyl)amino)-2-(2,2,2-trifluoroacetyl)-1,2,3,4-tetrahydroisoquinoline-8-carboxylate

To a solution of methyl 6-bromo-2-(2,2,2-trifluoroacetyl)-1,2,3,4-tetrahydroisoquinoline-8-carboxylate (1.5 g, 4.10 mmol) and tert-butyl methyl (2-(methylamino)ethyl)carbamate (1.16 g, 6.15 mmol, CAS #112257-19-9) in dioxane (30 mL) was added Cs₂CO₃ (2.40 g, 7.37 mmol), RuPhos Pd G3 (342 mg, 409 umol) at 20° C., under nitrogen flow. Then the reaction was stirred at 80° C., for 10 h under nitrogen atmosphere. On completion, the reaction was poured into water (30 mL) and extracted with EtOAc (30 mL×2). The combined organic phase is washed with brine (20 mL×2), and dried over Na₂SO₄. Then the mixture was filtered and concentrated to give a residue. The residue was purified by column chromatography on silica gel (eluted with petroleum ether:ethyl acetate=100:1 to 100:30) to give the title compound (1.7 g, 88% yield) as a yellow oil. ¹H NMR (400 MHZ, CDCl₃) δ=7.27-7.20 (m, 1H), 6.70-6.54 (m, 1H), 5.06 (d, J=15.1 Hz, 2H), 3.91 (s, 3H), 3.88-3.78 (m, 2H), 3.71 (s, 3H), 3.52 (br d, J=2.8 Hz, 2H), 3.43-3.31 (m, 2H), 2.99 (d, J=3.6 Hz, 3H), 2.95 (q, J=6.0 Hz, 2H), 1.45-1.38 (m, 9H).

### Step 6-Methyl 6-((2-((tert-butoxycarbonyl)(methyl)amino)ethyl)(methyl)amino)-1,2,3,4-tetrahydroisoquinoline-8-carboxylate

To a solution of methyl 6-((2-((tert-butoxycarbonyl)(methyl)amino)ethyl)(methyl)amino)-2-(2,2,2-trifluoroacetyl)-1,2,3,4-tetrahydroisoquinoline-8-carboxylate (2.20 g, 4.65 mmol) in THF (20 mL), H₂O (10 mL) and MeOH (10 mL) was added K₂CO₃ (770 mg, 5.58 mmol) at 20° C., under nitrogen flow. Then the reaction was stirred at 20° C., for 10 h under nitrogen atmosphere. On completion, the reaction was concentrated to give the title compound (2.4 g) as a yellow solid. LC-MS (ESI⁺) m/z 378.1 (M+H)⁺.

### Step 7—Methyl 2-(5-bromo-6-(tert-butoxycarbonyl)pyri din-2-yl)-6-((2-((tert-butoxycarbonyl)(methyl)amino)ethyl)(methyl)amino)-1,2,3,4-tetrahydroisoquinoline-8-carboxylate

To a solution of methyl 6-((2-((tert-butoxycarbonyl)(methyl)amino)ethyl)(methyl)amino)-1,2,3,4-tetrahydroisoquinoline-8-carboxylate (2.4 g, 6.36 mmol) and tert-butyl 3-bromo-6-fluoropicolinate (2.11 g, 7.63 mmol) in DMSO (30 mL) was added DIEA (4.11 g, 31.79 mmol) at 20° C., under nitrogen flow. Then the reaction was stirred at 60° C., for 10 h under nitrogen atmosphere. On completion, the reaction was poured into water (30 mL) and extracted with EtOAc (30 mL×2). The combined organic phase is washed with brine (20 mL×2), and dried over Na₂SO₄. Then the mixture was filtered and concentrated to give a residue. The residue was purified by column chromatography on silica gel (eluted with petroleum ether: ethyl acetate=100:1 to 100:15) to give the title compound (2.2 g, 55% yield) as a yellow solid. ¹H NMR (400 MHZ, CDCl₃) δ=7.51 (d, J=9.2 Hz, 1H), 7.17 (br s, 1H), 6.58 (d, J=9.2 Hz, 2H), 4.77 (s, 2H), 3.90-3.74 (m, 5H), 3.52-3.38 (m, 2H), 3.32 (br d, J=6.0 Hz, 2H), 2.91 (s, 3H), 2.85 (br t, J=6.0 Hz, 2H), 2.83-2.73 (m, 3H), 1.56 (s, 9H), 1.34 (br s, 9H).

### 6-(8-(benzo[d]thiazol-2-ylcarbamoyl)-6-(methyl (2-(methylamino)ethyl)amino)-3,4-dihydroisoquinolin-2 (1H)-yl)-3-(1-(cyclohexylmethyl)-5-methyl-1H-pyrazol-4-yl)picolinic acid (Intermediate DU)

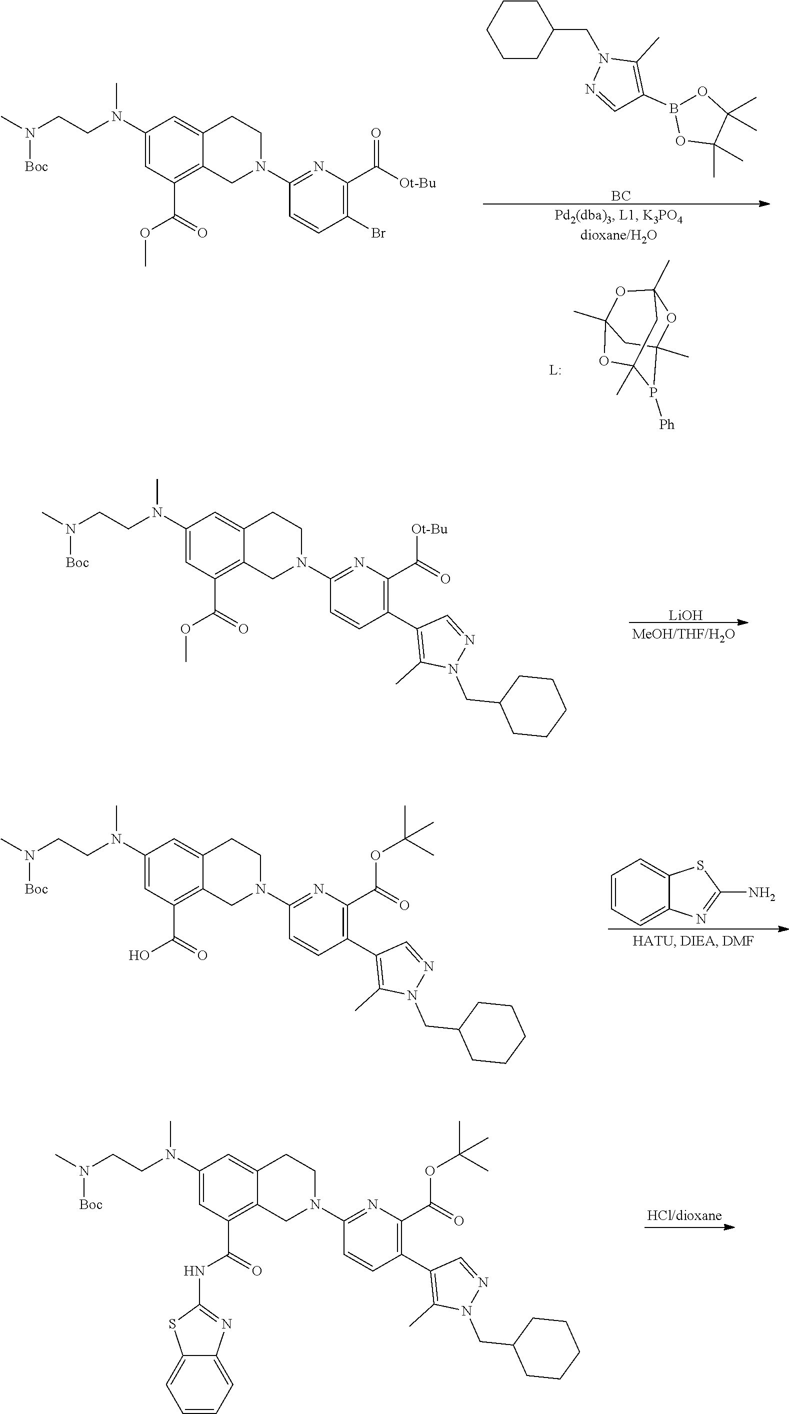

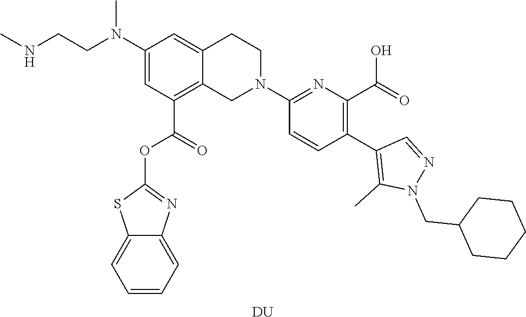 [SMILES: CNCCN(C)c1cc2c(c(C(=O)Oc3nc4ccccc4s3)c1)CN(c1ccc(-c3cnn(CC4CCCCC4)c3C)c(C(=O)O)n1)CC2 ; "DU"]

### Step 1—Methyl 6-((2-((tert-butoxycarbonyl)(methyl)amino)ethyl)(methyl)amino)-2-(6-(tert-butoxycarbonyl)-5-(1-(cyclohexylmethyl)-5-methyl-1H-pyrazol-4-yl) pyridin-2-yl)-1,2,3,4-tetrahydroisoquinoline-8-carboxylate

To a solution of methyl 2-(5-bromo-6-(tert-butoxycarbonyl) pyridin-2-yl)-6-((2-((tert-butoxycarbonyl)(methyl)amino)ethyl)(methyl)amino)-1,2,3,4-tetrahydroisoquinoline-8-carboxylate (2.2 g, 3.47 mmol, Intermediate DT) and 1-(cyclohexylmethyl)-5-methyl-4-(4,4,5,5-tetramethyl-1,3,2-dioxaborolan-2-yl)-1H-pyrazole (1.58 g, 5.21 mmol, Intermediate BC) in dioxane (20 mL) and water (20 mL) was added Pd (dba) 2 (199 mg, 347.24 umol), (3R,5S)-1,3,5,7-tetramethyl-8-phenyl-2,4,6-trioxa-8-phosphaadamantane (406 mg, 1.39 mmol) and K₃PO₄ (2.21 g, 10.4 mmol) at 20° C., under nitrogen flow. Then the reaction was stirred at 100° C., for 2 h under nitrogen atmosphere. On completion, the reaction was poured into water (40 mL) and extracted with EtOAc (30 mL×2). The combined organic phase was washed with brine (20 mL×2), and dried over Na₂SO₄. Then the mixture was filtered and concentrated to give a residue. The residue was purified by column chromatography on silica gel (eluted with petroleum ether: ethyl acetate=100:1 to 100:15) to give the title compound (2.4 g, 95% yield) as a colorless oil. ¹H NMR (400 MHZ, CDCl₃) δ=7.40 (s, 1H), 7.36 (d, J=8.8 Hz, 1H), 7.26-7.21 (m, 1H), 6.82 (br d, J=8.4 Hz, 1H), 6.77-6.63 (m, 1H), 4.90 (s, 2H), 4.02 (br t, J=5.6 Hz, 2H), 3.98-3.81 (m, 6H), 3.61-3.46 (m, 2H), 3.40 (br s, 2H), 3.02-2.94 (m, 5H), 2.93-2.80 (m, 3H), 2.14 (s, 3H), 2.00-1.89 (m, 2H), 1.78-1.60 (m, 8H), 1.42 (br s, 9H), 1.39 (s, 9H).

### Step 2—6-((2-((Tert-butoxycarbonyl)(methyl)amino)ethyl)(methyl)amino)-2-(6-(tert-butoxycarbonyl)-5-(1-(cyclohexylmethyl)-5-methyl-1H-pyrazol-4-yl) pyridin-2-yl)-1,2,3,4-tetrahydroisoquinoline-8-carboxylic acid

To a solution of methyl 2-[6-tert-butoxycarbonyl-5-[1-(cyclohexylmethyl)-5-methyl-pyrazol-4-yl]-2-pyridyl]-6-[2-[tert-butoxycarbonyl(methyl)amino]ethyl-methyl-amino]-3,4-dihydro-1H-isoquinoline-8-carboxylate (1 g, 1.37 mmol) in THF (10 mL), MeOH (5 mL) and H₂O (5 mL) was added LiOH·H₂O (287.03 mg, 6.84 mmol) at 20° C., under nitrogen flow. Then the reaction was stirred at 20° C. for 5 h under nitrogen atmosphere. On completion, the reaction was poured into water (20 mL) and extracted with EtOAc (30 mL×2). The combined organic phase is washed with brine (30 mL×2), and dried over Na₂SO₄. Then the mixture was filtered and concentrated to give the title compound (900 mg. 89% yield) as a colorless oil. ¹H NMR (400 MHZ, CDCl₃) δ=7.14 (br s, 2H), 6.68-6.52 (m, 1H), 6.41-6.14 (m, 2H), 4.95-4.71 (m, 2H), 3.85-3.70 (m, 4H), 3.24-3.02 (m, 4H), 2.68 (br d, J=2.0 Hz, 8H), 1.94-1.74 (m. 6H), 1.70-1.53 (m, 8H), 1.27 (br s, 9H), 1.18 (br d, J=4.4 Hz, 9H).

### Step 3—Tert-butyl 6-(8-(benzo[d]thiazol-2-ylcarbamoyl)-6-((2-((tert-butoxycarbonyl)(methyl)amino)ethyl)(methyl)amino)-3,4-dihydroisoquinolin-2 (1H)-yl)-3-(1-(cyclohexylmethyl)-5-methyl-1H-pyrazol-4-yl)picolinate

To a solution of 1,3-benzothiazol-2-amine (282.84 mg, 1.88 mmol, CAS #102337-98-4) and 6-((2-((tert-butoxycarbonyl)(methyl)amino)ethyl)(methyl)amino)-2-(6-(tert-butoxycarbonyl)-5-(1-(cyclohexylmethyl)-5-methyl-1H-pyrazol-4-yl) pyridin-2-yl)-1,2,3,4-tetrahydroisoquinoline-8-carboxylic acid (900 mg, 1.26 mmol) in DMF (18 mL) was added DIEA (486 mg. 3.77 mmol) and HATU (525 mg, 1.38 mmol) at 20° C., under nitrogen flow. Then the reaction was stirred at 50° C., for 10 h under nitrogen atmosphere. On completion, the reaction was poured into water (40 mL) then filtered and concentrated to give the title compound (0.8 g, 68% yield) as a colorless oil. ¹H NMR (400 MHZ, CDCl₃) δ 7.84-7.67 (m. 1H), 7.46-7.37 (m, 1H), 7.34-7.20 (m, 4H), 6.88-6.64 (m, 2H), 6.61-6.45 (m, 1H), 4.89-4.71 (m, 2H), 4.08-3.91 (m, 2H), 3.78 (br d, J=7.2 Hz, 2H), 3.41 (d, J=1.6 Hz, 1H), 3.39-3.29 (m, 2H), 3.23 (br s, 1H), 2.98-2.72 (m, 8H), 2.04 (s, 3H), 1.88-1.73 (m, 2H), 1.68-1.51 (m, 6H), 1.48-1.22 (m, 21H).

### Step 4—6-(8-(benzo[d]thiazol-2-ylcarbamoyl)-6-(methyl (2-(methylamino)ethyl)amino)-3,4-dihydroisoquinolin-2 (1H)-yl)-3-(1-(cyclohexylmethyl)-5-methyl-1H-pyrazol-4-yl)picolinic acid

To a solution of tert-butyl 6-(8-(benzo[d]thiazol-2-ylcarbamoyl)-6-((2-((tert-butoxycarbonyl)(methyl)amino)ethyl)(methyl)amino)-3,4-dihydroisoquinolin-2 (1H)-yl)-3-(1-(cyclohexylmethyl)-5-methyl-1H-pyrazol-4-yl)picolinate (0.8 g, 942 umol) in HCl/dioxane (4 M, 20 mL) at 20° C., under nitrogen flow. Then the reaction was stirred at 20° C., for 10 h under nitrogen atmosphere. On completion, the reaction was concentrated to give the title compound (800 mg) as a yellow solid. LC-MS (ESI⁺) m/z 693.3 (M+H)⁺. (S)-2-(1-aminoethyl)-5-(4-methylthiazol-5-yl)phenol (Intermediate DV)

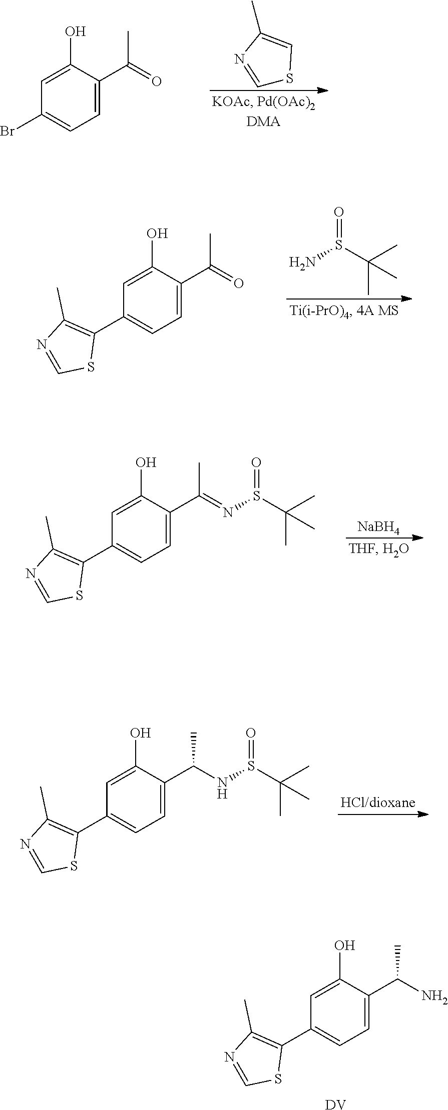

### Step 1—1-(2-Hydroxy-4-(4-methylthiazol-5-yl)phenyl)ethanone

To a solution of 1-(4-bromo-2-hydroxy-phenyl)ethanone (20.0 g, 93.0 mmol), 4-methylthiazole (18.4 g, 186 mmol) and KOAc (18.3 g, 186 mmol) in DMA (140 mL) was added Pd (OAc) 2 (1.04 g, 4.65 mmol) under N₂. Then the mixture was stirred at 120° C., for 12 h. On completion, the reaction mixture was poured into ice water (300 mL) and extracted with ethyl acetate (300 mL×3). The combined organic layers were washed with brine (100 mL×5), dried over sodium sulfate, filtered and concentrated under reduced pressure to give a residue. The residue was purified by column chromatography (SiO₂, Petroleum ether/Ethyl acetate=40/1 to 10/1) to afford title compound (20.0 g, 92% yield) as a yellow solid. ¹H NMR (400 MHZ, CHLOROFORM-d) δ=12.37 (s, 1H), 8.74 (s, 1H), 7.79 (d, J=8.4 Hz, 1H), 7.10 (d, J=1.6 Hz, 1H), 7.01 (dd, J=1.6, 8.4 Hz, 1H), 2.67 (s, 3H), 2.61 (s, 3H).

### Step 2—(R,E)-N-(1-(2-hydroxy-4-(4-methylthiazol-5-yl)phenyl)ethylidene)-2-methylpropane-2-sulfinamide

To a solution of 1-(2-hydroxy-4-(4-methylthiazol-5-yl)phenyl)ethanone (500 mg. 2.14 mmol), (R)-2-methylpropane-2-sulfinamide (779 mg. 6.43 mmol) and 4A molecular sieves (500 mg) in 2-methyltetrahydrofuran (10 mL) was added Ti (i-PrO)₄ (1.83 g, 6.43 mmol) at 0° C., under N₂. Then the mixture was heated to 80° C., for 2 h. On completion, the reaction mixture was diluted with EtOAc (50 mL) and H₂O (10 mL) and filtered. The mixture was diluted with water (10 mL), and extracted with EtOAc (20 mL×3). The combined organic layers were washed with brine (20 mL×3), dried over Na₂SO₄ and evaporated. The residue was purified by column chromatography (SiO₂, Petroleum ether/Ethyl acetate=3/1 to 2/1) to afford title compound (4 g, 56% yield) as a yellow solid. LC-MS (ESI) m/z 337.0 (M+H).

### Step 3—(R)-N-((S)-1-(2-hydroxy-4-(4-methylthiazol-5-yl)phenyl)ethyl)-2-methylpropane-2-sulfinamide

To a solution of (R,E)-N-(1-(2-hydroxy-4-(4-methylthiazol-5-yl)phenyl)ethylidene)-2-methylpropane-2-sulfinamide (4.00 g, 11.9 mmol) in THF (39.2 mL) and H₂O (0.8 mL) was added NaBH₄ (1.85 g, 48.9 mmol) at −50° C., under N₂. The mixture was stirred at 25° C., for 12 h and 40° C., for 12 h. On completion, the reaction mixture was quenched with H₂O (50 mL), and extracted with EtOAc (50 mL×3). The combined organic layers were washed with brine (40 mL×3), dried over Na₂SO₄ and evaporated. The residue was purified by prep-HPLC (column: Phenomenex luna C18 250*50 mm*15 um; mobile phase: [water (NH₃H₂O)-ACN]: B %: 15%-55%, 21 min) and reversed-phase HPLC (ACN/0.1% NH₃·H₂O=50%) to afford title compound (1.3 g, 32% yield) as a white solid. ¹H NMR (400 MHZ, CHLOROFORM-d) δ=8.65 (s, 1H), 8.45 (br s, 1H), 7.20 (d, J=8.0 Hz, 1H), 7.00 (d, J=1.6 Hz, 1H), 6.92 (dd, J=1.6, 8.0 Hz, 1H), 4.65 (dd, J=5.2, 6.8 Hz, 1H), 4.14 (d, J=5.2 Hz, 1H), 2.51 (s, 3H), 1.62 (d, J=6.8 Hz, 3H), 1.28 (s, 9H).

### Step 4—(S)-2-(1-aminoethyl)-5-(4-methylthiazol-5-yl)phenol

To a solution of (R)-N-((S)-1-(2-hydroxy-4-(4-methylthiazol-5-yl)phenyl)ethyl)-2-methylpropane-2-sulfinamide (1.10 g, 3.25 mmol) in dioxane (1.2 mL) was added HCl/dioxane (4 M, 11.0 mL). The mixture was stirred at 25° C., for 1 h. On completion, the reaction mixture was evaporated. The crude product was purified by reversed-phase HPLC (ACN/0.1% HCl=0%) to afford title compound (550 mg, HCl salt) as a white solid. LC-MS (ESI) m/z 235.1 (M+H).

### 2-(2-((S)-1-((2S,4R)-1-((S)-2-(1-fluorocyclopropanecarboxamido)-3,3-dimethylbutanoyl)-4-hydroxypyrrolidine-2-carboxamido) ethyl)-5-(4-methylthiazol-5-yl)phenoxy)acetic acid (Intermediate DW)

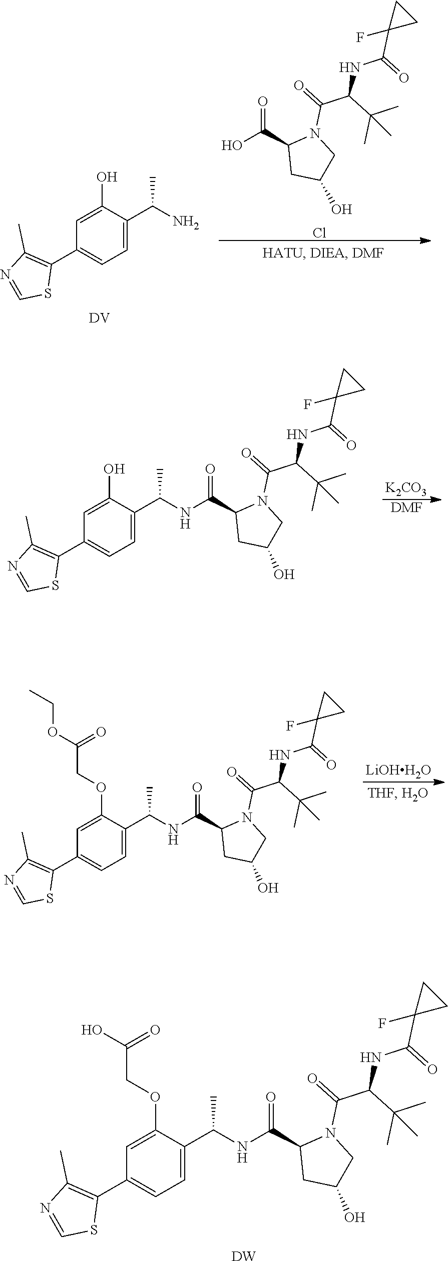

### Step 1—(2S,4R)-1-((S)-2-(1-fluorocyclopropanecarboxamido)-3,3-dimethylbutanoyl)-4-hydroxy-N-((S)-1-(2-hydroxy-4-(4-methylthiazol-5-yl)phenyl)ethyl)pyrrolidine-2-carboxamide

A solution of (2S,4R)-1-((S)-2-(1-fluorocyclopropanecarboxamido)-3,3-dimethylbutanoyl)-4-hydroxypyrrolidine-2-carboxylic acid (1.03 g, 3.12 mmol, Intermediate CI) and HATU (1.37 g, 3.60 mmol) in DMF (6.5 mL) was stirred for 0.5 h at 25° C. Then a mixture of DIEA (1.55 g, 12.0 mmol) and(S)-2-(1-aminoethyl)-5-(4-methylthiazol-5-yl)phenol (650 mg, 2.40 mmol, HCl salt, Intermediate DV) in DMF (3.2 mL) was added. The mixture was stirred at 25° C., for 12 h. On completion, the reaction mixture was purified by reversed-phase HPLC without work up. The crude product was purified by reversed-phase HPLC (ACN/0.1% FA condition=50% to 60%) to afford title compound (950 mg, 72% yield) as a yellow solid. ¹H NMR (400 MHZ, DMSO-d₆) δ=9.75 (s, 1H), 8.94 (s, 1H), 8.43 (d, J=7.6 Hz, 1H), 8.13 (s, 1H), 7.48 (d, J=8.0 Hz, 1H), 7.27 (br dd, J=2.0, 9.2 Hz, 1H), 6.89 (d, J=1.6 Hz, 1H), 6.84-6.78 (m, 1H), 5.15 (d, J=3.6 Hz, 1H), 4.96 (t, J=7.2 Hz, 1H), 4.60-4.51 (m, 2H), 4.34 (br s, 1H), 3.63-3.55 (m, 2H), 2.44 (s, 3H), 2.09-2.01 (m, 1H), 1.92 (ddd, J=4.4, 8.4, 12.8 Hz, 1H), 1.39 (br d, J=8.8 Hz, 1H), 1.31 (d, J=6.8 Hz, 3H), 1.21 (br dd, J=3.6, 8.8 Hz, 2H), 0.96 (br d, J=6.8 Hz, 1H), 0.89 (s, 9H).

### Step 2—Ethyl 2-(2-((S)-1-((2S,4R)-1-((S)-2-(1-fluorocyclopropanecarboxamido)-3,3-dimethylbutanoyl)-4-hydroxypyrrolidine-2-carboxamido) ethyl)-5-(4-methylthiazol-5-yl)phenoxy)acetate

To a solution of (2S,4R)-1-((S)-2-(1-fluorocyclopropanecarboxamido)-3,3-dimethylbutanoyl)-4-hydroxy-N-((S)-1-(2-hydroxy-4-(4-methylthiazol-5-yl)phenyl)ethyl)pyrrolidine-2-carboxamide (200 mg, 366 umol) and K₂CO; (152 mg, 1.10 mmol) in DMF (2 mL) was added ethyl 2-bromoacetate (91.7 mg, 549 umol). Then the mixture was stirred at 60° C., for 12 h. On completion, the reaction mixture was diluted with water (10 mL), and extracted with EtOAc (15 mL×3). The combined organic layers were washed with brine (5 mL×4), dried over Na₂SO₄ and evaporated to afford the title compound (240 mg) as a yellow oil. LC-MS (ESI⁺) m/z 633.3 (M+H)⁺.

### Step 3 2-(2-((S)-1-((2S,4R)-1-((S)-2-(1-fluorocyclopropanecarboxamido)-3,3-dimethylbutanoyl)-4-hydroxypyrrolidine-2-carboxamido) ethyl)-5-(4-methylthiazol-5-yl)phenoxy)acetic acid

To a solution of ethyl 2-(2-((S)-1-((2S,4R)-1-((S)-2-(1-fluorocyclopropanecarboxamido)-3,3-dimethylbutanoyl)-4-hydroxypyrrolidine-2-carboxamido) ethyl)-5-(4-methylthiazol-5-yl)phenoxy)acetate (240 mg, 379 umol) in THF (2 mL) and H₂O (1 mL) was added LiOH·H₂O (53.0 mg, 1.26 mmol). Then the mixture was stirred at 25° C., for 2 h. On completion, the reaction mixture was diluted with water (5 mL) and the pH was adjusted to 4˜3 by addition 2 M HCl, then extracted with EtOAc (10 mL×3). The combined organic layers were washed with brine (4 mL×3), dried over Na₂SO₄ and evaporated to afford the title compound (200 mg) as a yellow oil. ¹H NMR (400 MHZ, DMSO-d₆) δ=13.29-12.88 (m, 1H), 8.97 (s, 1H), 8.49 (br d, J=7.6 Hz, 1H), 7.57 (d, J=7.6 Hz, 1H), 7.30-7.26 (m, 1H), 6.96 (d, J=8.0 Hz, 1H), 6.90 (s, 1H), 5.03 (br t, J=6.8 Hz, 1H), 4.81 (d, J=5.2 Hz, 2H), 4.58-4.53 (m, 2H), 4.37-4.33 (m, 1H), 3.61-3.55 (m, 2H), 2.44 (s, 3H), 2.09-2.03 (m, 1H), 1.96-1.91 (m, 1H), 1.38-1.32 (m, 5H), 1.24-1.19 (m, 3H), 0.88 (s, 9H).

### (2S,4R)-4-hydroxy-1-((S)-2-(7-(4-(3-(1-((4-iodo-5-methyl-1H-pyrazol-1-yl)methyl)cyclohexyl)propyl)piperazin-1-yl)-7-oxoheptanamido)-3,3-dimethylbutanoyl)-N-((S)-1-(4-(4-methylthiazol-5-yl)phenyl)ethyl)pyrrolidine-2-carboxamide (Intermediate DX)

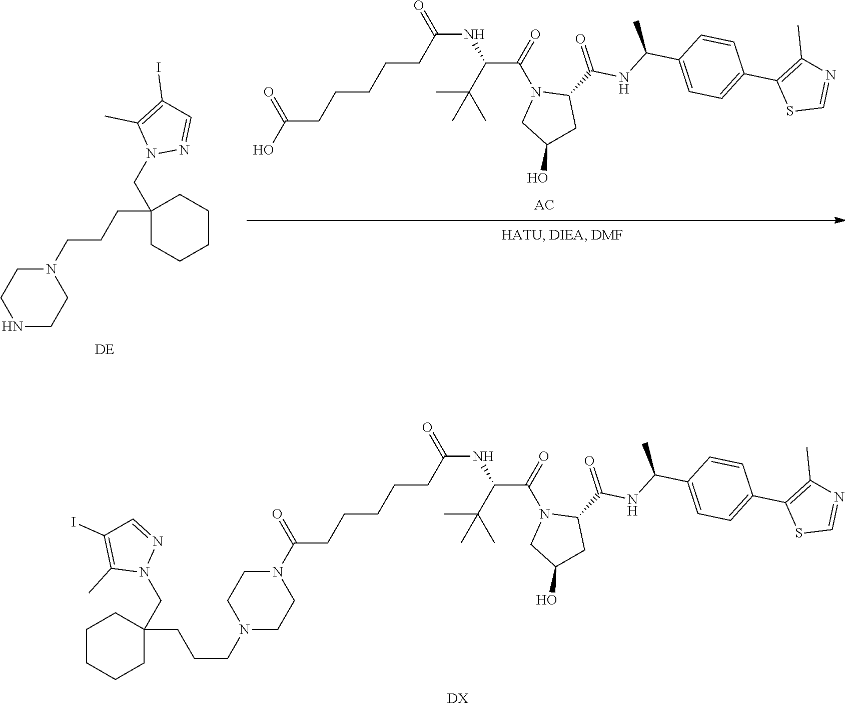

To a solution of 1-(3-(1-((4-iodo-5-methyl-1H-pyrazol-1-yl)methyl)cyclohexyl)propyl)piperazine (200 mg, 464 umol, Intermediate DE) and 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid (272 mg, 464 umol, Intermediate AC) in DMF (2 mL) was added to HATU (265 mg, 697 umol) and DIEA (300 mg, 2.32 mmol), then the mixture was stirred at 25° C. for 2 h. On completion, the reaction mixture was quenched with NH₄Cl (3 mL) and then extracted with EtOAc (5 mL×3). The combined organic layers were washed with brine (5 mL×3), dried over Na₂SO₄, filtered and concentrated under reduced pressure to give a residue. The residue was purified by column chromatography (SiO₂, DCM:MeOH=10:1) to give the title compound (270 mg, 58% yield) as a white solid. ¹H NMR (400 MHZ, DMSO-d₆) δ=8.98 (s, 1H), 8.36 (d, J=8.0 Hz, 1H), 7.83-7.74 (m, 1H), 7.47-7.34 (m, 5H), 5.75 (s, 1H), 5.09 (d, J=3.6 Hz, 1H), 4.95-4.89 (m, 1H), 4.51 (d, J=9.6 Hz, 1H), 4.42 (t, J=8.0 Hz, 1H), 4.28 (d, J=2.4 Hz, 1H), 3.93 (s, 1H), 3.64-3.57 (m, 2H), 3.40 (d, J=4.4 Hz, 3H), 3.17 (d, J=5.2 Hz, 4H), 2.45 (s, 3H), 2.37-2.17 (m, 10H), 2.16-2.09 (m, 1H), 2.03-1.95 (m, 1H), 1.79 (ddd, J=4.8, 8.4, 12.8 Hz, 1H), 1.54-1.18 (m, 23H), 0.93 (s, 9H).

### Ethyl 3-bromo-6-[(4-tert-butoxy-4-oxo-butyl)amino]pyridine-2-carboxylate (Intermediate DY)

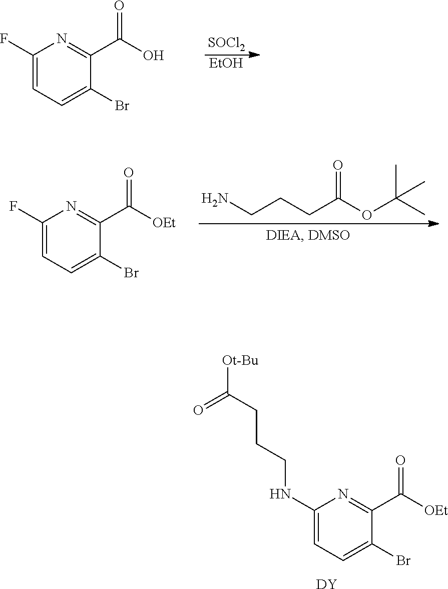

### Step 1—Ethyl 3-bromo-6-fluoro-pyridine-2-carboxylate

To a solution of 3-bromo-6-fluoro-pyridine-2-carboxylic acid (800 mg, 3.64 mmol, CAS #1211589-43-3) in EtOH (15 mL) was added DMF (53.1 mg, 728 umol) and SOCl₂ (2.16 g, 18.2 mmol), then the reaction mixture was stirred at 50° C., for 2 hrs. On completion, the reaction mixture was concentrated in vacuo. The residue was purified by column chromatography (SiO₂, PE/EtOAc=20/1) to give the title compound (720 mg, 80% yield) as colorless oil. ¹H NMR (400 MHZ, DMSO-d₆) δ 8.42 (dd, J=7.2, 8.8 Hz, 1H), 7.41 (dd, J=3.2, 8.8 Hz, 1H), 4.38 (q, J=7.2 Hz, 2H), 1.33 (t, J=7.2 Hz, 3H). Step 2—Ethyl 3-bromo-6-[(4-tert-butoxy-4-oxo-butyl)amino]pyridine-2-carboxylate

To a solution of ethyl 3-bromo-6-fluoro-pyridine-2-carboxylate (700 mg, 2.82 mmol) and tert-butyl 4-aminobutanoate (584 mg, 3.67 mmol, CAS #50479-22-6) in DMSO (10 mL) was added DIEA (1.09 g, 8.47 mmol), then the reaction mixture was stirred at 50° C., for 48 hrs. On completion, the reaction mixture was diluted with EtOAc (70 mL), the mixture was washed with brine (40 mL×4), dried over NaSO₄ and concentrated in vacuo. The residue was purified by column chromatography (SiO₂, PE/EtOAc=5/1, P1: Rf-0.7, PE/EtOAc=3/1) to give the title compound (650 mg, 60% yield) as colorless oil. ¹H NMR (400 MHz, CDCl₃-d) δ 7.58 (d, J=8.8 Hz, 1H), 6.38 (d, J=8.8 Hz, 1H), 4.88 (br s, 1H), 4.43 (q, J=7.2 Hz, 2H), 3.31 (q, J=6.4 Hz, 2H), 2.32 (t, J=7.2 Hz, 2H), 1.88 (quin, J=7.2 Hz, 2H), 1.53-1.36 (m, 12H).

### Ethyl 6-[(4-tert-butoxy-4-oxo-butyl)amino]-3-[1-(cyclohexylmethyl)-5-methyl-pyrazol-4-yl]pyridine-2-carboxylate (Intermediate DZ)

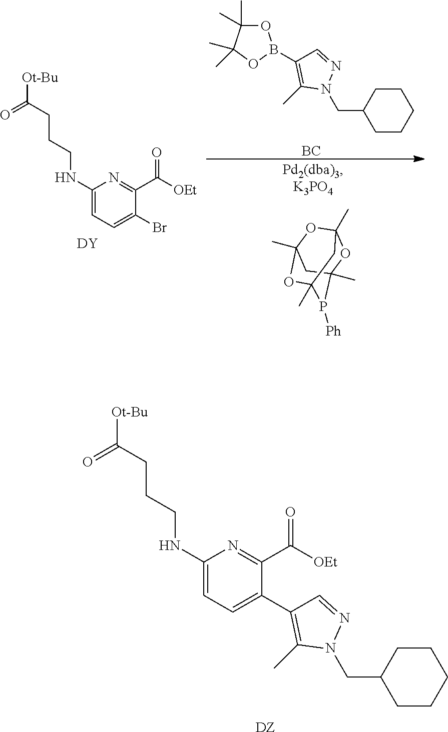

To a solution of ethyl 3-bromo-6-[(4-tert-butoxy-4-oxo-butyl)amino]pyridine-2-carboxylate (450 mg, 1.16 mmol, Intermediate DY) and 1-(cyclohexylmethyl)-5-methyl-4-(4,4,5,5-tetramethyl-1,3,2-dioxaborolan-2-yl) pyrazole (459 mg, 1.51 mmol, Intermediate BC) in dioxane (5 mL) and H₂O (5 mL) was added (3R,5S)-1,3,5,7-tetramethyl-8-phenyl-2,4,6-trioxa-8-phosphaadamantane (136 mg, 465 umol), K₃PO₄ (740 mg, 3.49 mmol) and Pd₂(dba); (106 mg, 116 umol). Then the reaction mixture was stirred at 100° C., for 4 hrs under N₂ atmosphere. On completion, the reaction mixture was extracted with EtOAc (150 mL), and the organic layer was washed with brine (70 mL×3). The organic layer was dried over Na₂SO₄ and concentrated in vacuo. The residue was purified by column chromatography (SiO₂, PE/EtOAc=5/1 to 2/1: P1: Rf=0.5, PE/EtOAc=1/1) to give the title compound (380 mg, 67% yield) as yellow oil. ¹H NMR (400 MHz, DMSO-d₆) δ 7.31 (d, J=8.8 Hz, 1H), 7.19 (s, 1H), 6.82 (d, J=5.2 Hz, 1H), 6.59 (d, J=8.8 Hz, 1H), 4.07-4.01 (m, 2H), 3.85 (d, J=7.2 Hz, 2H), 3.22 (q, J=6.4 Hz, 2H), 2.28 (t, J=7.6 Hz, 2H), 2.09 (s, 3H), 1.81-1.70 (m, 3H), 1.70-1.59 (m, 3H), 1.52 (d, J=12.4 Hz, 2H), 1.39 (s, 9H), 1.20-1.14 (m, 3H), 1.07-1.03 (m, 3H), 1.02-0.93 (m, 2H).

### N-(6-chloro-4-methyl-pyridazin-3-yl)-N-(2-trimethylsilylethoxymethyl)-1,3-benzothiazol-2-amine (Intermediate EA)

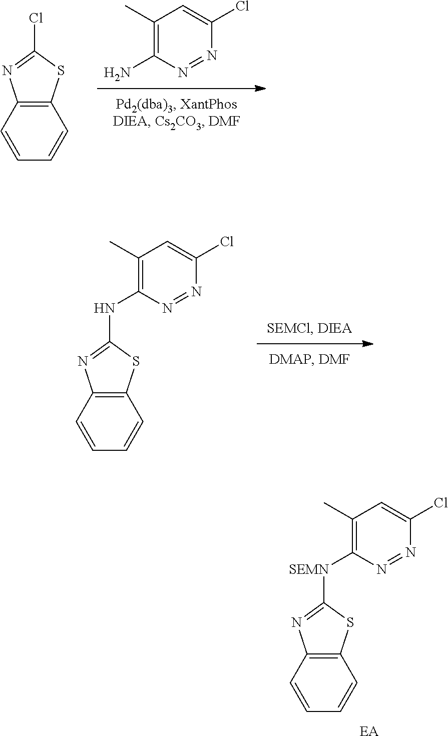

### Step 1—N-(6-chloro-4-methylpyridazin-3-yl)benzo[d]thiazol-2-amine

To a solution of 6-chloro-4-methyl-pyridazin-3-amine (5 g, 34.8 mmol, CAS #64068-00-4) and 2-chloro-1,3-benzothiazole (6.50 g, 38.3 mmol, CAS #615-20-3) in DMF (100 mL) was added DIEA (13.5 g, 104 mmol, 18.2 mL), Cs₂CO₃ (34.0 g, 104 mmol), Pd₂(dba); (1.59 g, 1.74 mmol) and XantPhos (2.02 g, 3.48 mmol). The mixture was stirred at 75° C., for 4 h under N₂ atmosphere. On completion, the reaction mixture was poured into sat. NH₄Cl (100 mL) and extracted with EtOAc (200 mL×3). The combined organic layers were washed with brine (100 mL×8), dried over sodium sulfate, filtered and concentrated under reduced pressure to give a residue. The residue was purified by flash silica gel chromatography (ISCOR: 120 g SepaFlash® Silica Flash Column, Eluent of 0˜30% Ethyl acetate/Petroleum ether gradient (a 80 mL/min) to give the title compound (4.9 g, 51% yield) as a green solid. ¹H NMR (400 MHZ, DMSO-d₆) δ 13.54-10.71 (m, 1H), 7.92-7.83 (m, 1H), 7.69-7.63 (m, 1H), 7.61-7.47 (m, 1H), 7.40 (br t, J=7.6 Hz, 1H), 7.23 (br t, J=7.6 Hz, 1H), 2.38 (s, 3H).

### Step 2—N-(6-chloro-4-methylpyridazin-3-yl)-N-((2-(trimethylsilyl)ethoxy)methyl)benzo[d]thiazol-2-amine

To a solution of N-(6-chloro-4-methyl-pyridazin-3-yl)-1,3-benzothiazol-2-amine (4.4 g, 15.90 mmol) in DMF (80 mL) was added DIEA (6.16 g, 47.70 mmol, 8.31 mL), and then 2-(chloromethoxy)ethyl-trimethyl-silane (7.95 g, 47.7 mmol, 8.44 mL) and DMAP (388 mg, 3.18 mmol) was added at 0° C. The mixture was stirred at 20° C., for 12 h. On completion, the reaction mixture was poured into ice water (100 mL) and extracted with EtOAc (200 mL×3). The combined organic layers were washed with brine (200 mL×5), dried over sodium sulfate, filtered and concentrated under reduced pressure to give a residue. The residue was purified by flash silica gel chromatography (ISCOR; 80 g SepaFlash® Silica Flash Column, Eluent of 0˜10% Ethyl acetate/Petroleum ethergradient @ 80 mL/min) to give the title compound (2.5 g, 32% yield) as a yellow solid. ¹H NMR (400 MHZ, DMSO-d₆) δ 7.83 (d, J=7.6 Hz, 1H), 7.67 (d, J=0.8 Hz, 1H), 7.52-7.42 (m, 2H), 7.30-7.24 (m, 1H), 5.86 (s, 2H), 3.70 (t, J=7.8 Hz, 2H), 2.37 (s, 3H), 0.92-0.86 (m, 2H), 0.13 (s, 9H).

### 4-[[6-(1,3-benzothiazol-2-ylamino)-5-methyl-pyridazin-3-yl]-[5-[1-(cyclohexylmethyl)-5-methyl-pyrazol-4-yl]-6-ethoxycarbonyl-2-pyridyl]amino]butanoic acid (Intermediate EB)

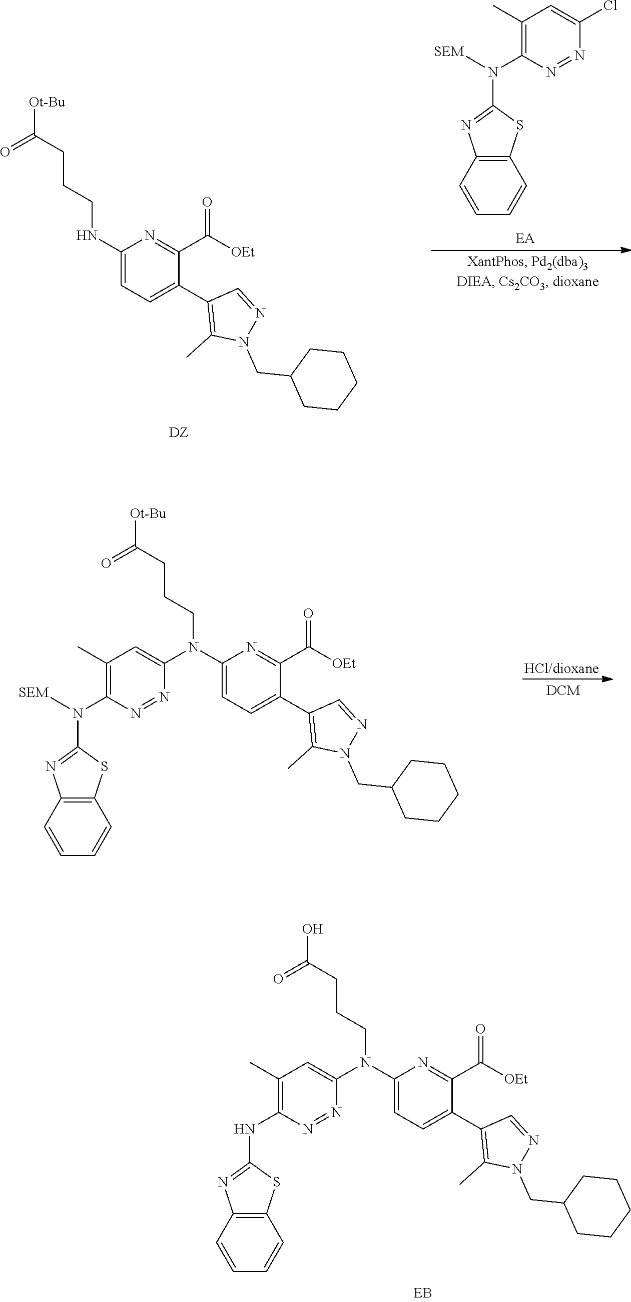 [SMILES: CCOC(=O)c1nc(NCCCC(=O)OC(C)(C)C)ccc1-c1cnn(CC2CCCCC2)c1C ; Cc1cc(Cl)nnc1N(COCC[Si](C)(C)C)c1nc2ccccc2s1 ; CCOC(=O)c1nc(N(CCCC(=O)OC(C)(C)C)c2cc(C)c(N(COCC[Si](C)(C)C)c3nc4ccccc4s3)nn2)ccc1-c1cnn(CC2CCCCC2)c1C ; CCOC(=O)c1nc(N(CCCC(=O)O)c2cc(C)c(Nc3nc4ccccc4s3)nn2)ccc1-c1cnn(CC2CCCCC2)c1C]

### Step 1—Ethyl 6-[[6-[1,3-benzothiazol-2-yl (2-trimethylsilylethoxymethyl)amino]-5-methyl-pyridazin-3-yl]-(4-tert-butoxy-4-oxo-butyl)amino]-3-[1-(cyclohexylmethyl)-5-methyl-pyrazol-4-yl]pyridine-2-carboxylate

To a solution of ethyl 6-[(4-tert-butoxy-4-oxo-butyl)amino]-3-[1-(cyclohexylmethyl)-5-methyl-pyrazol-4-yl]pyridine-2-carboxylate (380 mg, 784 umol, Intermediate DZ) and N-(6-chloro-4-methyl-pyridazin-3-yl)-N-(2-trimethylsilylethoxymethyl)-1,3-benzothiazol-2-amine (415 mg, 1.02 mmol, Intermediate EA) in dioxane (15 mL) was added Xantphos (90.7 mg, 157 umol), Pd₂(dba); (71.8 mg, 78.4 umol), DIEA (304 mg, 2.35 mmol) and Cs₂CO; (766 mg, 2.35 mmol). Then the mixture was stirred at 120° C., for 12 hrs under N₂. On completion, the reaction mixture was diluted with EtOAc (70 mL), filtered and the filtrate was washed with brine (30 mL×2), dried over Na₂SO₄, filtered and concentrated in vacuo. The residue was purified by column chromatography (SiO₂, PE/EtOAc=5/1 to 2/1: P1: Rf=0.65, PE/EtOAc=1/1) to give the title compound (400 mg, 57% yield) as yellow solid. ¹H NMR (400 MHZ, DMSO-d₆) δ 7.79 (d, J=7.6 Hz, 1H), 7.62 (d, J=8.4 Hz, 1H), 7.57 (d, J=0.8 Hz, 1H), 7.50-7.40 (m, 2H), 7.32 (s, 1H), 7.28-7.22 (m, 1H), 7.20 (d, J=8.8 Hz, 1H), 5.92-5.82 (m, 2H), 4.20-4.18 (m, 2H), 4.14-4.09 (m, 2H), 3.89 (d, J=7.2 Hz, 2H), 3.78-3.68 (m, 2H), 2.38-2.29 (m, 5H), 2.16 (s, 3H), 1.96-1.88 (m, 2H), 1.81-1.79 (m, 1H), 1.71-1.59 (m, 3H), 1.54 (d, J=10.8 Hz, 2H), 1.37 (s, 9H), 1.24-1.15 (m, 3H), 1.10 (t, J=7.1 Hz, 3H), 1.04-0.96 (m, 2H), 0.95-0.88 (m, 2H), −0.09-0.14 (m, 9H).

### Step 2—4-[[6-(1,3-Benzothiazol-2-ylamino)-5-methyl-pyridazin-3-yl]-[5-[1-(cyclohexylmethyl)-5-methyl-pyrazol-4-yl]-6-ethoxycarbonyl-2-pyridyl]amino]butanoic acid

To a solution of ethyl 6-[6-[1,3-benzothiazol-2-yl (2-trimethylsilylethoxymethyl)amino]-5-methyl-pyridazin-3-yl]-(4-tert-butoxy-4-oxo-butyl)amino]-3-[1-(cyclohexylmethyl)-5-methyl-pyrazol-4-yl]pyridine-2-carboxylate (100 mg, 117 umol) in DCM (0.2 mL) was added HCl/dioxane (1 mL), then the reaction was stirred at 40° C., for 2 hrs. On completion, the reaction was concentrated in vacuo to give the title compound (90 mg, 98% yield) as yellow solid. LC-MS (ESI⁺) m/z 669.3 (M+H)⁺.

### 4-[4-[2-(4-chlorophenyl)-5,5-dimethyl-cyclohexen-1-yl]methyl]piperazin-1-yl]benzoic acid (CAS #1044598-91-5) (Intermediate EC)

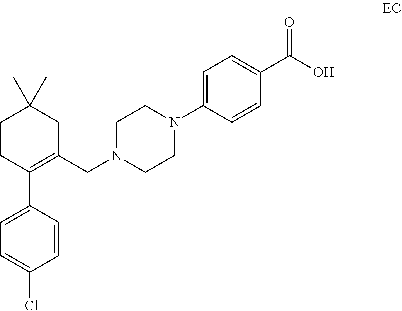 [SMILES: CC1(C)CCC(c2ccc(Cl)cc2)=C(CN2CCN(c3ccc(C(=O)O)cc3)CC2)C1]

### 2-(aminomethyl)-5-ethynylphenol (Intermediate ED)

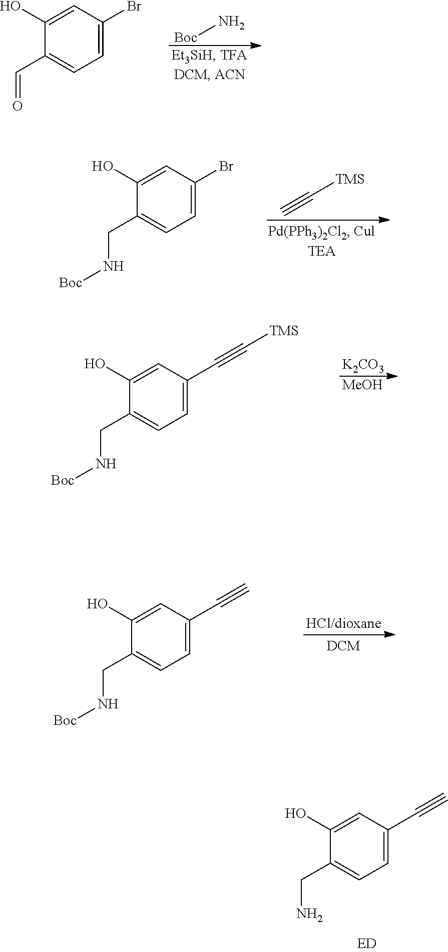

### Step 1—Tert-butyl (4-bromo-2-hydroxybenzyl)carbamate

To a solution of 4-bromo-2-hydroxybenzaldehyde (95.0 g, 472 mmol) and tert-butyl carbamate (83.04 g, 708 mmol) in DCM (100 mL) and ACN (300 mL) was added Et; SiH (164 g, 1.41 mol, 226 mL) and TFA (107 g, 945 mmol, 70 mL). The mixture was stirred at 25° C., for 24 hrs. On completion, the reaction mixture was quenched with NaHCO; (1000 mL) at 25° C., and then diluted with water (500 mL) and extracted with DCM (800 mL×2). The combined organic layers were washed with brine (1000 mL), dried over Na₂SO₄, filtered and concentrated under reduced pressure to give a residue. The crude product was triturated with n-heptane (500 mL) at 25° C., for 30 min, and filtered to give the title compound (130 g, 91% yield) as a yellow solid. LC-MS (ESI⁺) m/z 247.9 (M+H)⁺. ¹H NMR (400 MHZ, CDCl₃) ò 7.12-7.11 (m, 1H), 6.96-6.90 (m, 2H), 5.28 (s, 1H), 4.43 (s, 1H), 4.18-4.17 (m, 2H), 1.46 (s, 9H).

### Step 2—Tert-butyl (2-hydroxy-4-((trimethylsilyl)ethynyl)benzyl)carbamate

To a solution of tert-butyl (4-bromo-2-hydroxybenzyl)carbamate (130 g, 430 mmol) and ethynyltrimethylsilane (129 g, 1.31 mol, 182 mL) was added CuI (4.16 g, 21.8 mmol) and TEA (727 g, 7.18 mol, 1 L), Pd (dppf) Cl₂ (15.6 g, 21.3 mmol). The mixture was stirred at 80° C., for 5 hrs. The reaction mixture was filtered and concentrated under reduced pressure to give a residue. The residue was purified by flash silica gel chromatography (ISCOR: 800 g SepaFlash® Silica Flash Column, Eluent of 0˜100% Ethyl acetate/Petroleum ether gradient (@ 60 mL/min) to give the title compound (90 g, 65% yield) as a yellow solid. LC-MS (ESI⁺) m/z 240.0 (M+H)⁺. ¹H NMR (400 MHZ, CDCl₃) ò 8.98 (s, 1H), 7.03-6.92 (m, 3H), 5.33-5.29 (m, 1H), 4.21-4.19 (m, 2H), 1.45 (s, 9H), 0.25 (s, 9H).

### Step 3—Tert-butyl (4-ethynyl-2-hydroxybenzyl)carbamate

To a solution of Tert-butyl (2-hydroxy-4-((trimethylsilyl)ethynyl)benzyl)carbamate (45.0 g, 140 mmol) in MeOH (500 mL) was added K₂CO₃ (38.9 g, 281 mmol). The mixture was stirred at 25° C. for 12 hrs. On completion, the reaction mixture was filtered and concentrated under reduced pressure to give a residue. The residue was dissolved with DCM (800 mL) and adjusted to pH=6˜7 with HCl (IM). The organic layer was washed with water (500 mL×2), dried over Na₂SO₄, filtered and concentrated under reduced pressure to give the title compound (60.0 g, 86% yield) as a brown solid. LC-MS (ESI⁺) m/z 192.0 (M+H)⁺. ¹H NMR (400 MHZ, CDCl₃) ò 9.08 (s, 1H), 7.03-6.92 (m, 3H), 5.31 (s, 1H), 4.22-4.20 (m, 2H), 1.45 (s, 9H).

### Step 4—2-(aminomethyl)-5-ethynylphenol

To a solution of Tert-butyl (4-ethynyl-2-hydroxybenzyl)carbamate (60.0 g, 242 mmol) in DCM (300 mL) was added HCl/dioxane (4 M, 120 mL). The mixture was stirred at 25° C., for 12 hrs. On completion, the reaction mixture was filtered to give the title compound (40 g, 90% yield, HCl) as a yellow solid. LC-MS (ESI⁺) m/z 131.0 (M+H)⁺. ¹H NMR (400 MHZ, DMSO) δ 10.62 (s, 1H), 7.24-7.32 (m, 1H), 7.08 (s, 1H), 6.95-6.93 (m, 1H), 4.18 (s, 1H), 3.93-3.91 (m, 2H).

### Methyl (2S,4R)-1-((S)-2-amino-3,3-dimethylbutanoyl)-4-hydroxypyrrolidine-2-carboxylate (Intermediate EE)

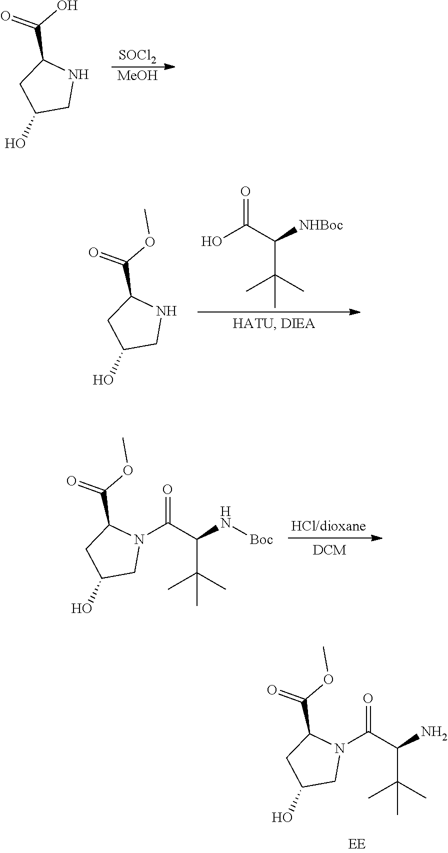

### Step 1—Methyl (2S,4R)-4-hydroxypyrrolidine-2-carboxylate

To a solution of (2S,4R)-4-hydroxypyrrolidine-2-carboxylic acid (200 g, 1.53 mol) in MeOH (1.00 L) was added SOCl₂ (229 g, 1.93 mol, 140 mL) and DMF (950 mg, 13.0 mmol, 1 mL) at 0° C. The mixture was stirred at 25° C., for 10 hrs and 50° C., for 2 hrs. On completion, the reaction mixture was concentrated under reduced pressure to give a residue. The crude product was triturated with MTBE (1000 mL×2) at 25° C., for 30 mins, and filtered to give the title compound (230 g, 83% yield, HCl) as a white solid. ¹H NMR (400 MHZ, DMSO) & 4.49-4.42 (m, 2H), 3.75 (s 3H), 3.31-3.30 (m, 1H), 3.09-3.06 (m, 1H), 2.22-2.17 (m, 1H), 2.12-2.09 (m, 1H).

### Step 2—Methyl (2S,4R)-1-((S)-2-((tert-butoxycarbonyl)amino)-3,3-dimethylbutanoyl)-4-hydroxypyrrolidine-2-carboxylate

To a solution of methyl (2S,4R)-4-hydroxypyrrolidine-2-carboxylate (230 g, 1.27 mol, HCl) in DCM (1500 mL) was added HATU (520 g, 1.37 mol) and DIEA (530 g, 4.10 mol, 700 mL) and(S)-2-((tert-butoxycarbonyl)amino)-3,3-dimethylbutanoic acid (358 g, 1.55 mol) at 0° C. The mixture was stirred at 25° C., for 12 hrs. On completion, the reaction mixture was partitioned between water (2000 mL) and DCM (1000 mL). The organic phase was separated, washed with water (500 mL×2) and brine (500 mL), dried over Na₂SO₄, filtered and concentrated under reduced pressure to give a residue. The residue was purified by flash silica gel chromatography (ISCOR: 800 g SepaFlash® Silica Flash Column, Eluent of 0˜100% Ethyl acetate/Petroleum ether gradient (100 mL/min) to give the title compound (450 g, 99% yield) as a yellow oil. LC-MS (ESI⁺) m/z 359.1 (M+H)⁺. ¹H NMR (400 MHZ, CDCl₃) ¿ 5.32-5.29 (m, 1H), 4.66-4.65 (m, 1H), 4.49 (s, 1H), 4.21-4.18 (m, 1H), 4.08-3.89 (m, 1H), 3.71-3.68 (m, 4H), 2.36-2.31 (m, 1H), 1.98-1.97 (m, 1H), 1.39 (s, 9H), 1.02 (s, 9H).

### Step 3—Methyl (2S,4R)-1-((S)-2-amino-3,3-dimethylbutanoyl)-4-hydroxypyrrolidine-2-carboxylate

To a solution of Methyl (2S,4R)-1-((S)-2-((tert-butoxycarbonyl)amino)-3,3-dimethylbutanoyl)-4-hydroxypyrrolidine-2-carboxylate (200 g, 557 mmol) in DCM (500 mL) was added HCl/dioxane (4 M, 500 mL). The mixture was stirred at 25° C., for 12 hrs. On completion, the reaction mixture was concentrated under reduced pressure to give the title compound (300 g, 91% yield, HCl) as a white solid. LC-MS (ESI⁺) m/z 259.1 (M+H)⁺. ¹H NMR (400 MHZ, DMSO) δ 8.23 (s, 3H), 4.43-4.41 (m, 1H), 4.38-4.35 (m, 1H), 3.93-3.91 (m, 1H), 3.83-3.79 (m, 1H), 3.57-3.56 (m, 5H), 2.17-2.16 (m, 1H), 1.92-1.89 (m, 1H), 1.03 (s, 9H).

### (2S,4R)-1-((S)-2-(1-fluorocyclopropane-1-carboxamido)-3,3-dimethylbutanoyl)-4-hydroxypyrrolidine-2-carboxylic acid (Intermediate EF)

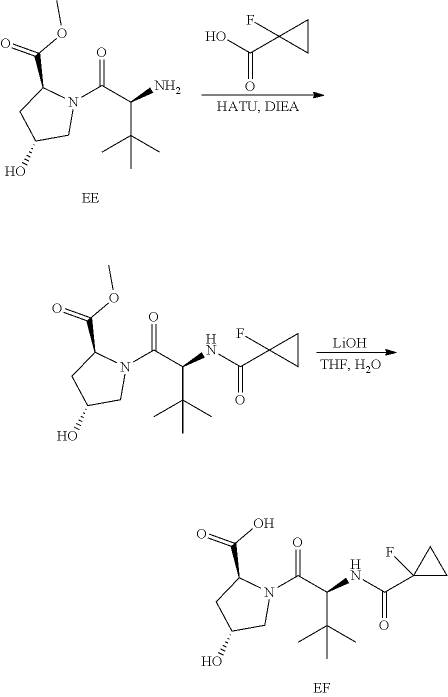

### Step 1 Methyl (2S,4R)-1-((S)-2-(1-fluorocyclopropane-1-carboxamido)-3,3-dimethylbutanoyl)-4-hydroxypyrrolidine-2-carboxylate

To a solution of methyl (2S,4R)-1-((S)-2-amino-3,3-dimethylbutanoyl)-4-hydroxypyrrolidine-2-carboxylate (150 g, 508 mmol, HCl, Intermediate EE) and 1-fluorocyclopropane-1-carboxylic acid (52.9 g. 508 mmol) in DCM (1200 mL) was added HATU (232 g, 610 mmol) and DIEA (200 g, 1.55 mol, 270 mL) at 0° C. The mixture was stirred at 25° C., for 6 hrs. On completion, the reaction mixture was partitioned between water (1000 mL) and DCM (1000 mL). The organic phase was separated, washed with water (1000 mL×2) and brine (1000 mL), dried over Na₂SO₄, filtered and concentrated under reduced pressure to give the title compound (300 g) as a brown oil. LC-MS (ESI⁺) m/z 345.1 (M+H)⁺.

### Step 2—(2S,4R)-1-((S)-2-(1-fluorocyclopropane-1-carboxamido)-3,3-dimethylbutanoyl)-4-hydroxypyrrolidine-2-carboxylic acid

To a solution of Methyl (2S,4R)-1-((S)-2-(1-fluorocyclopropane-1-carboxamido)-3,3-dimethylbutanoyl)-4-hydroxypyrrolidine-2-carboxylate (150 g, 435 mmol) in THF (800 mL) and H₂O (400 mL) was added LiOH·H₂O (91.5 g, 2.18 mol). The mixture was stirred at 25° C., for 3 hrs. On completion, the reaction mixture was concentrated under reduced pressure to remove THF. The residue was diluted with water (1000 mL) and extracted with ethyl acetate (500 mL×2). The aqueous phase was adjusted to pH=2˜3 with HCl (2M) and extracted with ethyl acetate (500 mL×2), dried over Na₂SO₄, filtered and concentrated under reduced pressure to give a residue. The residue was purified by flash silica gel chromatography (ISCOR: 2000 g SepaFlash®; Silica Flash Column, Eluent of 0˜100% Ethyl acetate/Petroleum ether gradient (a, 100 mL/min) to give the title compound (120 g, 42% yield) as a white solid. LC-MS (ESI⁺) m/z 331.0 (M+H)⁺. ¹H NMR (400 MHz, DMSO) δ 7.28-7.26 (m, 1H), 4.61-4.59 (m, 1H), 4.33-4.31 (m, 2H), 3.65-3.61 (m, 3H), 2.13-2.12 (m, 1H), 1.91-1.90 (m, 1H), 1.40-1.33 (m, 2H), 1.23-1.20 (m, 2H), 0.98 (s, 9H).

### Methyl 5-bromo-2-chlorothiazole-4-carboxylate (Intermediate EG)

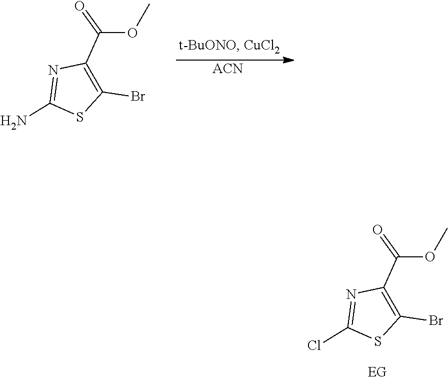

To a solution of methyl 2-amino-5-bromothiazole-4-carboxylate (50.0 g, 200 mmol) and CuCl (29.8 g, 300 mmol) in ACN (1500 mL) was added to tert-butyl nitrite (41.3 g, 401 mmol), then the mixture was stirred at 25° C., for 12 h. On completion, the reaction mixture was quenched with sat. NH₄Cl (500 mL) and then extracted with EtOAc (500 mL×3). The combined organic layers were washed with brine (500 mL×3), dried over Na₂SO₄, filtered and concentrated under reduced pressure to give a residue. The residue was purified by flash silica gel chromatography (Ethyl acetate/Petroleum ether=0 to 1/19) to give the title compound (40.0 g, 78% yield) as a yellow solid. ¹H NMR (400 MHz, DMSO-d₆) δ=3.84 (s, 3H).

### Methyl 2-(8-(benzo[d]thiazol-2-ylcarbamoyl)-3,4-dihydroisoquinolin-2 (1H)-yl)-5-bromothiazole-4-carboxylate (Intermediate EH)

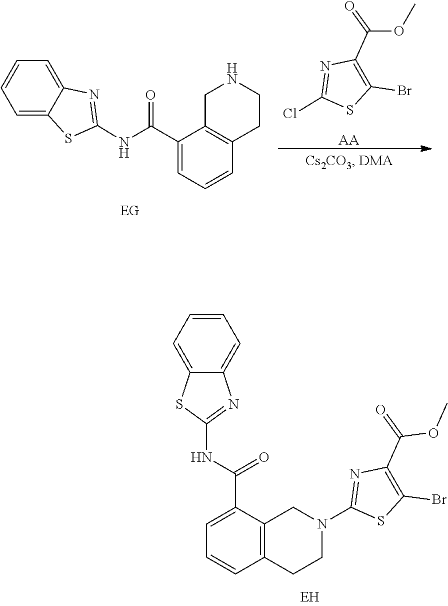

To a solution of N-(1,3-benzothiazol-2-yl)-1,2,3,4-tetrahydroisoquinoline-8-carboxamide (28.0 g, 90.5 mmol, Intermediate AA) and methyl 5-bromo-2-chloro-thiazole-4-carboxylate (30.2 g, 118 mmol, Intermediate EG) in DMA (500 mL) was added to Cs₂CO₃ (88.5 g, 272 mmol), then the mixture was stirred at 60° C., for 12 h. On completion, the reaction mixture was filtered to remove Cs₂CO₃, then filter liquor was quenched with sat. NH₄Cl (400 mL) and then extracted with DCM (400 mL×3). The combined organic layers were washed with brine (300 mL×6), dried over Na₂SO₄, filtered and concentrated under reduced pressure to give a residue. The residue was purified by flash silica gel chromatography (Ethyl acetate/Petroleum ether=0/1 to 1/1) to give the title compound (28.0 g, 58% yield) as a yellow solid. ¹H NMR (400 MHZ, DMSO-d₆) δ=12.90 (s, 1H), 8.03 (d, J=8.0 Hz, 1H), 7.79 (d, J=8.0 Hz, 1H), 7.69 (d, J=7.6 Hz, 1H), 7.51-7.30 (m, 5H), 4.86 (s, 2H), 3.74 (s, 3H), 3.71 (t, J=6.0 Hz, 2H).

### 6-(3-(benzo[d]thiazol-2-ylamino)-4-methyl-6,7-dihydropyrido[2,3-c]pyridazin-8 (5H)-yl)-3-(1-(cyclohexylmethyl)-5-methyl-1H-pyrazol-4-yl)picolinic acid (Intermediate EI)

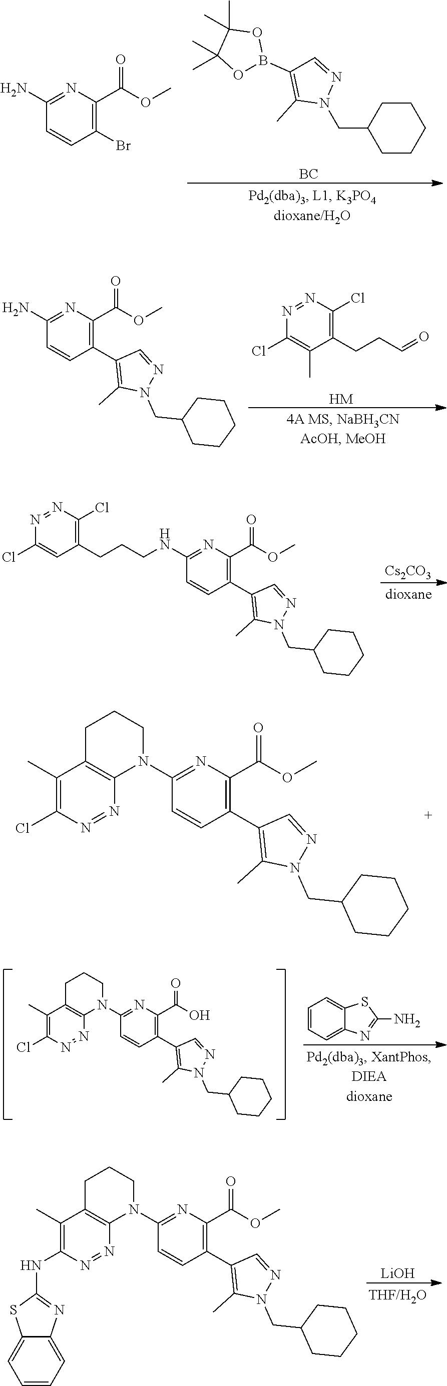

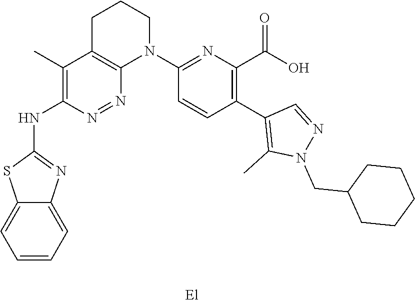 [SMILES: Cc1c(Nc2nc3ccccc3s2)nnc2c1CCCN2c1ccc(-c2cnn(CC3CCCCC3)c2C)c(C(=O)O)n1]

Step 1—Methyl 6-amino-3-(1-(cyclohexylmethyl)-5-methyl-1H-pyrazol-4-yl)picolinate. To a solution of methyl 6-amino-3-bromopicolinate (1.00 g, 4.33 mmol, CAS #178876-83-0) and 1-(cyclohexylmethyl)-5-methyl-4-(4,4,5,5-tetramethyl-1,3,2-dioxaborolan-2-yl)-1H-pyrazole (1.81 g, 4.76 mmol, Intermediate BC) in dioxane (8 mL) and H₂O (8 mL) was added Pd₂(dba)₃ (198 mg, 216 umol), K₃PO₄ (2.76 g, 13.0 mmol) and (1S,3R,5R,7S)-1,3,5,7-tetramethyl-8-phenyl-2,4,6-trioxa-8-phosphatricyclo[3,3,1,1^{3,7}]decane (253 mg, 866 umol, L1). Then the mixture was stirred at 100° C., for 4 h under N₂. On completion, the reaction mixture was quenched with NH₄Cl (8 mL) and then extracted with EtOAc (15 mL×3). The combined organic layers were washed with brine (8 mL×2), dried over Na₂SO₄, filtered and concentrated under reduced pressure to give a residue. The residue was purified by silica gel column chromatography (Petroleum ether:Ethyl acetate=10:1˜1:1) to give the title compound (1.42 g. quant, yield) as a white solid. ¹H NMR (400 MHZ, DMSO-d₆) δ 7.37-7.29 (m, 1H), 7.20 (s, 1H), 6.59 (dd. J=1.6, 8.4 Hz, 1H), 6.24 (br s, 2H), 5.75 (d, J=2.0 Hz, 1H), 3.93 (d, J=2.0 Hz, 1H), 3.85 (br d, J=6.8 Hz, 2H), 3.60 (d, J=1.6 Hz, 3H), 2.08 (s, 3H), 1.79 (br s, 1H), 1.69-1.58 (m, 3H), 1.50 (br d, J=12.0 Hz, 2H), 1.19-1.12 (m, 4H), 1.07 (d, J=2.0 Hz, 7H), 1.02-0.91 (m, 2H).

Step 2—Methyl 3-(1-(cyclohexylmethyl)-5-methyl-1H-pyrazol-4-yl)-6-((3-(3,6-dichloro-5-methylpyridazin-4-yl) propyl) amino)picolinate. To a solution of methyl 6-amino-3-(1-(cyclohexylmethyl)-5-methyl-1H-pyrazol-4-yl)picolinate (1.37 g, 4.17 mmol) and 3-(3,6-dichloro-5-methylpyridazin-4-yl) propanal (830 mg. 3.79 mmol, Intermediate HM) in McOH (10 mL) and AcOH (3.75 mL) was added 4A molecular sieves (1.00 g, 3.79 mmol), then the reaction was stirred at 25° C., for 1 h. Next, NaBH₃CN (1.19 g, 18.9 mmol) was added and the mixture was stirred at 85° C., for 1 h. On completion, the reaction mixture was quenched with NH₄Cl (15 mL) and extracted with EtOAC (15 mL×3). The combined organic layers were dried over Na₂SO₄ and evaporated. The crude product was purified by reversed-phase HPLC (0.1% FA condition, 5%-70%, 30 min) to give the title compound (1.17 g, 58% yield) as a white solid. ¹H NMR (400 MHZ, DMSO-d₆) δ 7.34 (d, J=8.8 Hz, 1H), 7.21 (s, 1H), 6.64 (d, J=8.8 Hz, 1H), 3.86 (d, J=7.6 Hz, 2H), 3.61 (s, 3H), 3.35 (br d, J=5.6 Hz, 1H), 2.90-2.83 (m, 2H), 2.41 (s, 3H), 2.09 (s, 3H), 1.83-1.74 (m, 3H), 1.74-1.56 (m, 4H), 1.53-1.46 (m, 2H), 1.26-1.07 (m, 4H), 1.01-0.90 (m, 2H).

Step 3—Methyl 6-(3-chloro-4-methyl-6,7-dihydropyrido]2,3-c]pyridazin-8 (5H)-yl)-3-(1-(cyclohexylmethyl)-5-methyl-1H-pyrazol-4-yl)picolinate & 6-(3-chloro-4-methyl-6,7-dihydropyrido]2,3-c]pyridazin-8 (5H)-xl)-3-(1-(cyclohexylmethyl)-5-methyl-1H-pyrazol-4-yl)picolinic acid. To a solution of methyl 3-(1-(cyclohexylmethyl)-5-methyl-1H-pyrazol-4-yl)-6-((3-(3,6-dichloro-5-methylpyridazin-4-yl) propyl) amino)picolinate (1.17 g, 2.20 mmol) in dioxane (10 mL) was added Cs₂CO₃ (3.59 g, 11.0 mmol), then mixture was stirred at 130° C., for 56 h. On completion, the solution was filtered and the reaction mixture was quenched with NH₄Cl (5 mL) and extracted with EtOAC (5 mL×3). The combined organic layers were dried over Na₂SO₄ and evaporated. The residue was purified by column chromatography (SiO₂. Petroleum ether/Ethyl acetate=10:1˜ 5:1) to give methyl 6-(3-chloro-4-methyl-6,7-dihydropyrido]2,3-c]pyridazin-8 (5H)-yl)-3-(1-(cyclohexylmethyl)-5-methyl-1H-pyrazol-4-yl)picolinate (300 mg. 28% yield) as a white solid (LC-MS (ESI) m/z 495.3 (M+H)) and 6-(3-chloro-4-methyl-6,7-dihydropyrido]2,3-c]pyridazin-8 (5H)-x1)-3-(1-(cyclohexylmethyl)-5-methyl-1H-pyrazol-4-yl)picolinic acid (200 mg, 19% yield) as a white solid (¹H NMR (400 MHZ, DMSO-d₆) δ 13.10-12.51 (m, 1H), 7.81 (d, J=8.8 Hz, 1H), 7.67 (d, J=8.8 Hz, 1H), 7.39 (s, 1H), 4.06-3.94 (m, 4H), 3.90 (d, J=7.2 Hz, 2H), 2.87 (br t, J=6.4 Hz, 2H), 2.29 (s, 3H), 2.19 (s, 3H), 1.99 (s, 4H), 1.91 (s, 1H), 1.87-1.75 (m, 2H), 1.70-1.61 (m, 4H), 1.55 (br d, J=11.6 Hz, 2H), 1.18-1.13 (m, 4H), 0.99 (br d, J=11.6 Hz, 2H)).

Step 4—Methyl 6-(3-chloro-4-methyl-6,7-dihydropyrido]2,3-c]pyridazin-8 (5H)-yl)-3-(1-(cyclohexylmethyl)-5-methyl-1H-pyrazol-4-yl)picolinate. To a stirred solution of 6-(3-chloro-4-methyl-6,7-dihydropyrido]2,3-c]pyridazin-8 (5H)-yl)-3-(1-(cyclohexylmethyl)-5-methyl-1H-pyrazol-4-yl)picolinic acid (200 mg, 416 umol) in MeOH (6 mL) at 0° C., was added SOCl₂ (495 mg, 4.16 mmol, 302 uL) dropwise. After addition, the mixture was stirred at 80° C., for 5 h. On completion, the solution was concentrated in vacuo. The crude product was purified by reversed-phase HPLC (ACN: 0.1% NH₃. H₂O=5%-100%, 30 min) to give the title compound (150 mg, 73% yield) as a white solid. LC-MS (ESI) m z 495.3 (M+H).

Step 5—Methyl 6-(3-(benzo[d]thiazol-2-ylamino)-4-methyl-6,7-dihydropyrido]2,3-c]pyridazin-8 (5H)-yl)-3-(1-(cyclohexylmethyl)-5-methyl-1H-pyrazol-4-yl)picolinate. To a solution of methyl 6-(3-chloro-4-methyl-6,7-dihydropyrido]2,3-c]pyridazin-8 (5H)-x1)-3-(1-(cyclohexylmethyl)-5-methyl-1H-pyrazol-4-yl)picolinate (270 mg, 545 umol) and benzo[d]thiazol-2-amine (492 mg, 3.2 mmol) in dioxane (5 mL) was added Xantphos (126 mg, 218 umol), Pd₂(dba); (99.9 mg, 109 umol), and DIEA (352 mg, 2.73 mmol, 475 uL) under N₂. The mixture was stirred at 120° C., for 36 h. On completion, the reaction mixture was quenched with NH₄Cl (5 mL) and extracted with EtOAc (5 mL×3). The combined organic layers were dried over Na₂SO₄ and evaporated. The residue was purified by column chromatography (SiO₂, Petroleum ether/Ethyl acetate=5:1˜1:1) to afford the title compound (250 mg. 75% yield) as white solid. LC-MS (ESI) m/z 609.0 (M+H).

Step 6-6-(3-(Benzo[d]thiazol-2-ylamino)-4-methyl-6,7-dihydropyrido]2,3-c]pyridazin-8 (5H)-yl)-3-(1-(cyclohexylmethyl)-5-methyl-1H-pyrazol-4-yl)picolinic acid. To a solution of methyl 6-(3-(benzo[d]thiazol-2-ylamino)-4-methyl-6,7-dihydropyrido]2,3-c]pyridazin-8 (5H)-yl)-3-(1-(cyclohexylmethyl)-5-methyl-1H-pyrazol-4-yl)picolinate (250 mg, 308 umol) in THF (5 mL) and H₂O (1 mL) was added to LiOH·H₂O (129 mg, 3.08 mmol), then the mixture was 25° C., for 30 h. On completion, the reaction mixture was filtered and the pH of the aqueous phase was adjusted to 4˜5 by addition 2M HCl and extracted with EtOAC (2 mL×2). The crude product was purified by reversed-phase HPLC (column: Waters xbridge 150*25 mm 10 um; mobile phase: [water (NH₄HCO₃)-ACN]: B %: 26%-56%, 8 min) to afford the title compound (43.7 mg, 24% yield) as yellow solid. LC-MS (ESI) m/z 595.2 (M+H); 1H NMR (400 MHZ, DMSO-d₆) δ 7.81 (br d, J=7.6 Hz, 2H), 7.59 (d, J=8.8 Hz, 1H), 7.53-7.47 (m, 1H), 7.45 (s, 1H), 7.37-7.32 (m, 1H), 7.19-7.13 (m, 1H), 4.03-3.99 (m, 2H), 3.89 (d, J=7.2 Hz, 2H), 2.85 (br t, J=6.4 Hz, 2H), 2.31 (s, 3H), 2.22 (s, 3H), 2.01-1.95 (m, 2H), 1.83 (dt, J=4.2, 7.2 Hz, 1H), 1.71-1.66 (m, 2H), 1.63 (br s, 1H), 1.57 (br d, J=10.8 Hz, 2H), 1.22-1.15 (m, 3H), 1.04-0.96 (m, 2H).

### Ethyl 1-(4-sulfamoylphenyl)piperidine-4-carboxylate (Intermediate EJ)

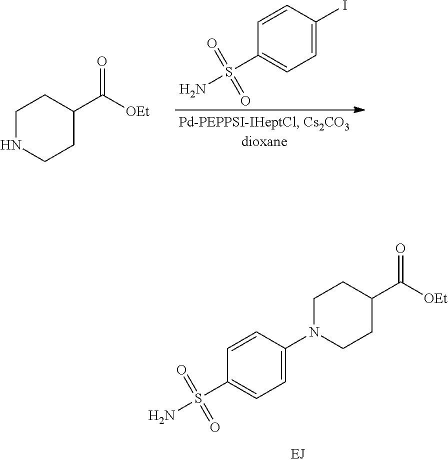 [SMILES: CCOC(=O)C1CCNCC1 ; NS(=O)(=O)c1ccc(I)cc1 ; CCOC(=O)C1CCN(c2ccc(S(N)(=O)=O)cc2)CC1]

A solution of 4-iodobenzenesulfonamide (500 mg, 1.77 mmol, CAS #825-86-5), ethyl piperidine-4-carboxylate (416 mg, 2.65 mmol, CAS #1126 Sep. 6), Cs₂CO₃ (1.73 g, 5.30 mmol) and Pd-PEPPSI-I HeptCl (171 mg, 176 umol) in dioxane (15 mL) was stirred at 100° C., under N₂ for 16 h. On completion, the reaction was filtered and the filtrate was concentrated in vacuo. The residue was purified by column chromatography (SiO₂, Petroleum ether/Ethyl acetate=3/1 to 1/1) to give the title compound (300 mg, 54% yield) as a white solid. LC-MS (ESI⁺) m/z 313.1 (M+H)⁺.

### 1-(4-(N-(6-(3-(benzo[d]thiazol-2-ylamino)-4-methyl-6,7-dihydropyrido[2,3-c]pyridazin-8 (5H)-yl)-3-(1-(cyclohexylmethyl)-5-methyl-1H-pyrazol-4-yl)picolinoyl)sulfamoyl)phenyl)piperidine-4-carboxylic acid (Intermediate EK)

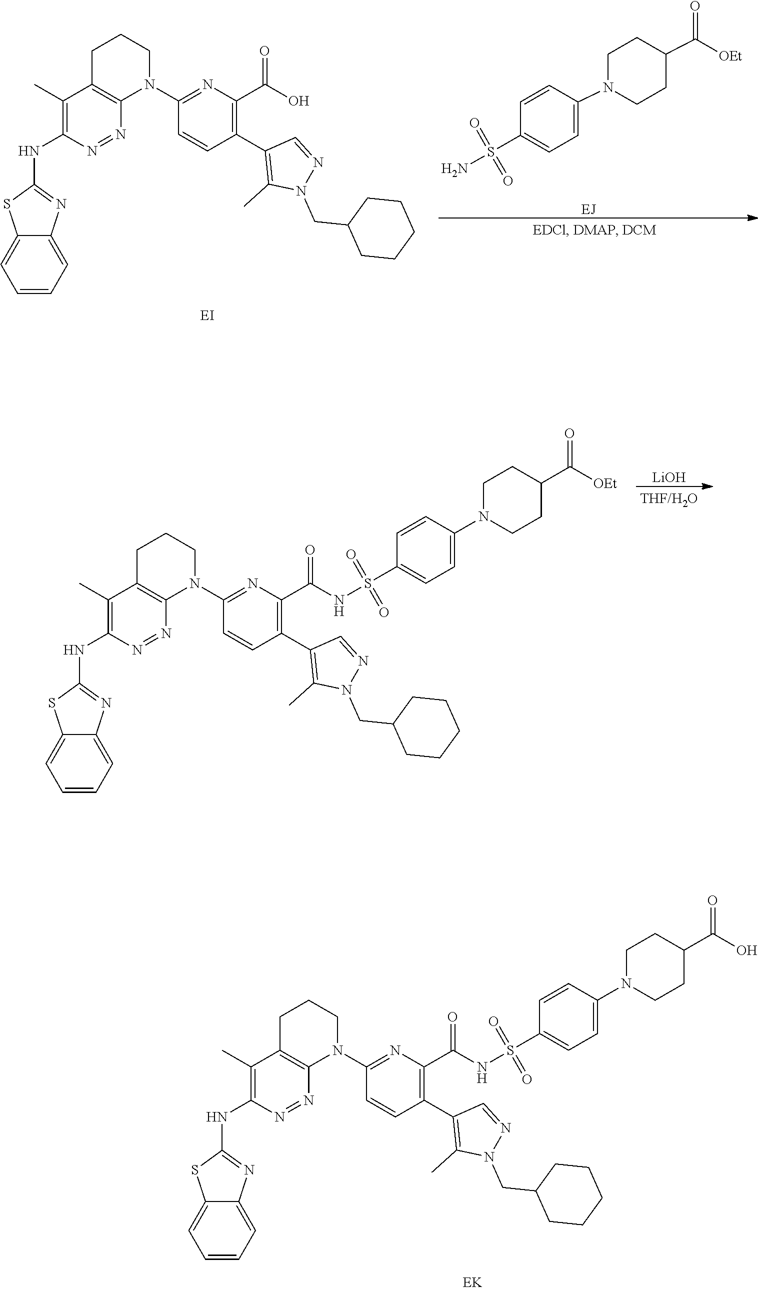

Step 1—Ethyl 1-(4-(N-(6-(3-(benzo[d]thiazol-2-ylamino)-4-methyl-6,7-dihydropyrido[2,3-c]pyridazin-8 (5H)-yl)-3-(1-(cyclohexylmethyl)-5-methyl-1H-pyrazol-4-yl)picolinoyl)sulfamoyl)phenyl)piperidine-4-carboxylate. To a solution of 6-(3-(benzo[d]thiazol-2-ylamino)-4-methyl-6,7-dihydropyrido[2,3-c]pyridazin-8 (5H)-yl)-3-(1-(cyclohexylmethyl)-5-methyl-1H-pyrazol-4-yl)picolinic acid (240 mg, 403 umol, Intermediate EI) and ethyl 1-(4-sulfamoylphenyl)piperidine-4-carboxylate (151 mg, 484 umol, Intermediate EJ) in DCM (3 mL) was added EDCI (154 mg, 807 umol) and DMAP (123 mg, 1.01 mmol). Then the mixture was stirred at 40° C., for 2 h. On completion, the solution was concentrated in vacuo. The crude product was purified by reversed-phase HPLC (0.1% FA condition, B %: 5%-80%, 30 min) to give the title compound (140.0 mg, 38% yield) as an orange solid. LC-MS (ESI⁺) m/z 889.7 (M+H)⁺.

Step 2—1-(4-(N-(6-(3-(benzo[d]thiazol-2-ylamino)-4-methyl-6,7-dihydropyrido[2,3-c]pyridazin-8 (5H)-yl)-3-(1-(cyclohexylmethyl)-5-methyl-1H-pyrazol-4-yl)picolinoyl)sulfamoyl)phenyl)piperidine-4-carboxylic acid. A solution of ethyl 1-(4-(N-(6-(3-(benzo[d]thiazol-2-ylamino)-4-methyl-6,7-dihydropyrido[2,3-c]pyridazin-8 (5H)-yl)-3-(1-(cyclohexylmethyl)-5-methyl-1H-pyrazol-4-yl)picolinoyl)sulfamoyl)phenyl)piperidine-4-carboxylate (140 mg, 157 umol) and LiOH·H₂O (33.0 mg, 787 umol) in THF (3 mL) and H₂O (1 mL) was stirred at 25° C., for 12 h. On completion, the reaction mixture was filtered and extracted with DCM (5 mL). The aqueous phase pH of the aqueous phase was adjusted to 4˜5 by addition 2M HCl and extracted with DCM (10 mL×3). The combined organic layers were dried over Na₂SO₄ and evaporated to give the title compound (140.0 mg) as an orange solid. LC-MS (ESI⁺) m/z 861.4 (M+H)⁺.

### 4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)butanoic acid (Intermediate EL)

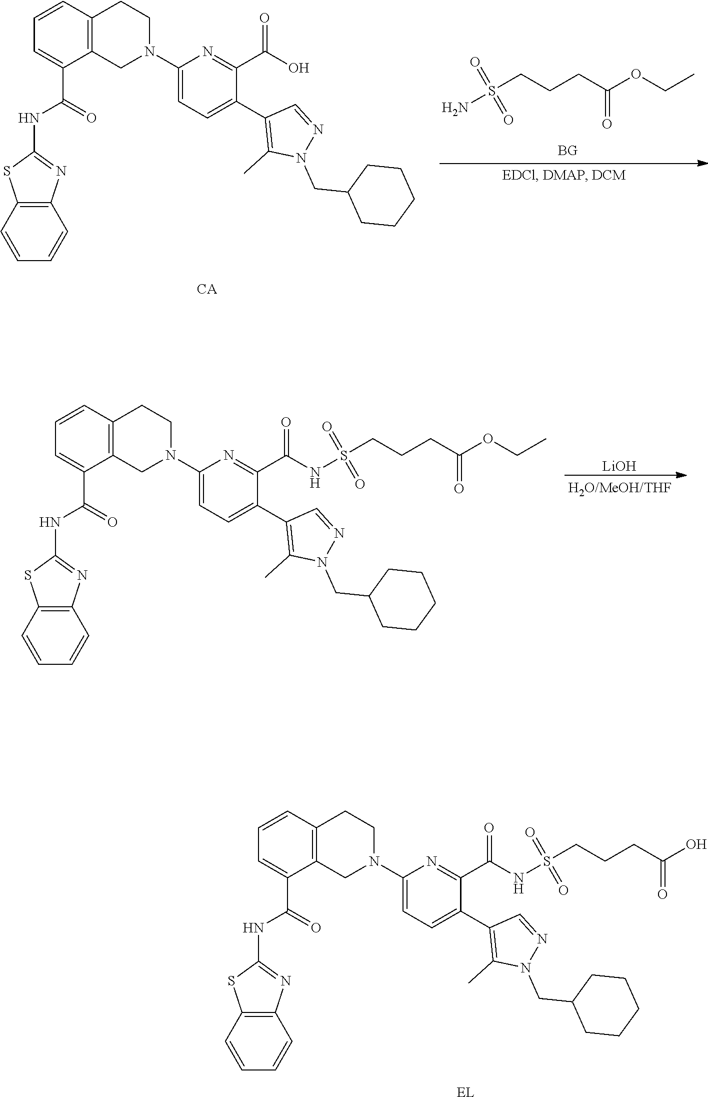 [SMILES: Cc1c(-c2ccc(N3CCc4cccc(C(=O)Nc5nc6ccccc6s5)c4C3)nc2C(=O)O)cnn1CC1CCCCC1 ; CCOC(=O)CCCS(N)(=O)=O ; CCOC(=O)CCCS(=O)(=O)NC(=O)c1nc(N2CCc3cccc(C(=O)Nc4nc5ccccc5s4)c3C2)ccc1-c1cnn(CC2CCCCC2)c1C ; "LiOH" ; Cc1c(-c2ccc(N3CCc4cccc(C(=O)Nc5nc6ccccc6s5)c4C3)nc2C(=O)NS(=O)(=O)CCCC(=O)O)cnn1CC1CCCCC1 ; "EL"]

Step 1—Ethyl 4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)butanoate. To a solution of ethyl 4-sulfamoylbutanoate (118 mg, 659 umol, Intermediate BG) and 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (200 mg, 329 umol, Intermediate CA) in DCM (2 mL) was added EDCI (126 mg, 659 umol) and DMAP (100 mg, 824 umol). The mixture was stirred at 40° C., for 12 h. On completion, the mixture was washed with H₂O (4 mL), and extracted with DCM (5 mL×3). Then the organic layer was with brine (10 ml×2), dried over anhydrous Na₂SO₄, filtered and concentrated under reduced pressure to give a residue. The residue was purified by column chromatography (SiO₂, Petroleum ether:Ethyl acetate=3:1 to Petroleum ether:Ethyl acetate=0:1) to give the title compound (120 mg, 46% yield) as a white solid. LC-MS (ESI⁺) m/z 784.4 (M+H)⁺.

Step 2—4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)butanoic acid. To a solution of ethyl 4-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]butanoate (120 mg, 153 umol) in THF (0.5 mL), H₂O (0.5 mL), and MeOH (0.5 mL) was added LiOH·H₂O (32 mg, 765 umol). The mixture was then stirred at 25° C., for 1 h. On completion, the mixture was adjusted to acidic pH then concentrated in vacuo. The mixture was washed with H₂O (3 mL), and extracted with EtOAc (2 mL×3). The combined organic layer was washed with brine (3 mL), dried over anhydrous Na₂SO₄, filtered and concentrated under reduced pressure to give a the title compound (40 mg) as a white solid. LC-MS (ESI⁺) m/z 756.4 (M+H)+.

### 3-(4-(N-(6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinoyl)sulfamoyl)phenyl)propanoic acid (Intermediate EM)

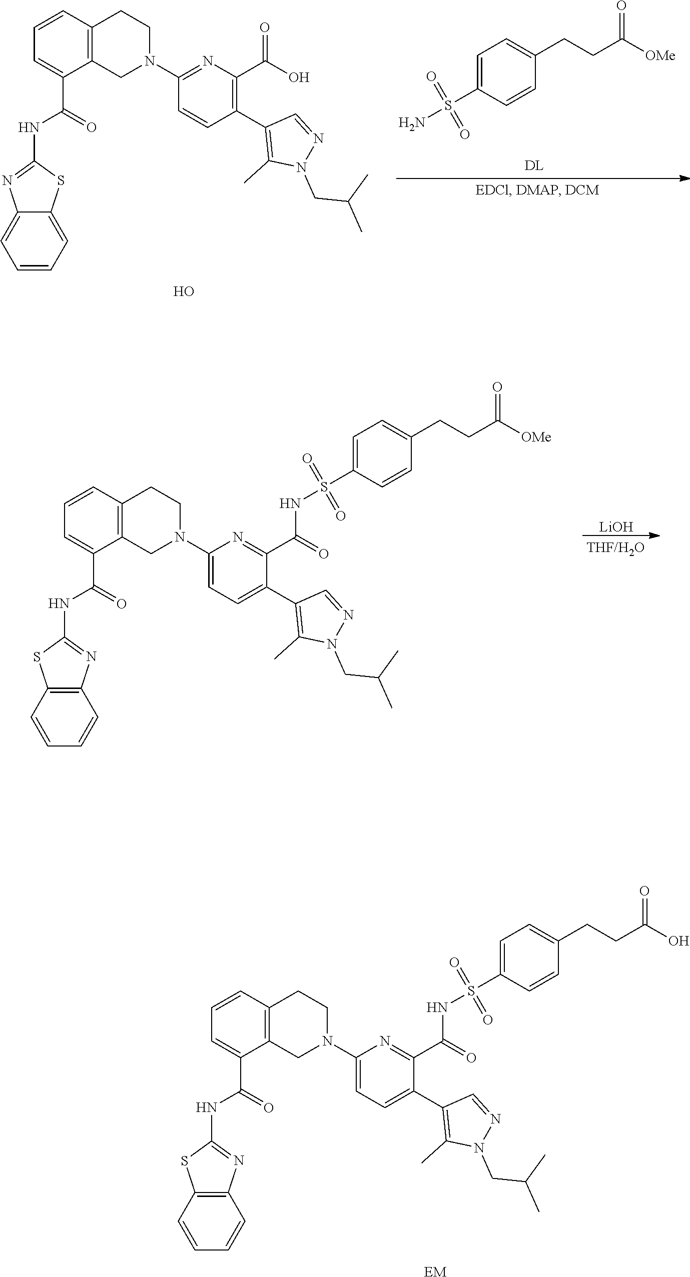

Methyl 3-(4-(N-(6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinoyl)sulfamoyl)phenyl)propanoate. To a solution of 6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinic acid (650 mg, 1.15 mmol, Intermediate HO), methyl 3-(4-sulfamoylphenyl)propanoate (335 mg, 1.38 mmol, Intermediate DL) in DCM (8 mL) was added EDCI (550 mg, 2.87 mmol) and DMAP (280 mg, 2.29 mmol). The mixture was then stirred at 25° C., for 12 h. On completion, the reaction was poured into water (10 mL) and extracted with EtOAc (10 mL×3). The combined organic phase was washed with brine (5 mL×2), and dried over Na₂SO₄. Then filtered to get the filtrate and concentrated to give a residue. The residue was purified by column chromatography (SiO₂, Petroleum ether/Ethyl acetate=5/1 to 0:1) to give the title compound (180 mg, 20% yield) as white solid. LC-MS (ESI⁺) m/z 792.1 (M+H)⁺.

Step 2—3-(4-(N-(6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinoyl)sulfamoyl)phenyl)propanoic acid. To a solution of methyl 3-(4-(N-(6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinoyl) sulfamoyl)phenyl)propanoate (180 mg, 227 umol) in THF (0.8 mL), H₂O (0.8 mL) and MeOH (0.8 mL) was added LiOH·H₂O (47.7 mg, 1.14 mmol). The mixture was stirred at 25° C., for 2 h. On completion, the pH of the reaction mixture was adjusted to 6˜5 with 2 M HCl, then the mixture was extracted with DCM (5 ml×5). The organic phase was separated, dried over Na₂SO₄, filtered and concentrated under reduced pressure to give the title compound (160 mg) as a white solid. LC-MS (ESI⁺) m/z 777.8 (M+H)⁺.

### (2S,4R)—N-(4-ethynylbenzyl)-4-hydroxypyrrolidine-2-carboxamide (Intermediate EN)

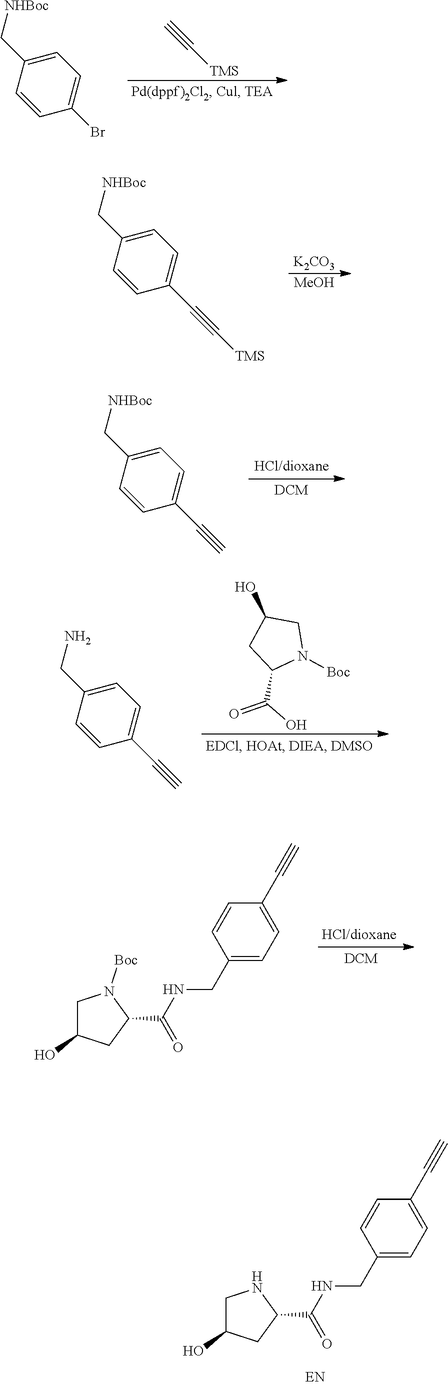

Step 1—Tert-butyl 4-((trimethylsilyl)ethynyl)benzylcarbamate. To a solution of tert-butyl 4-bromobenzylcarbamate (150 g, 524 mmol) and ethynyltrimethylsilane (155 g, 1.58 mol) in TEA (1.50 L) was added Pd (dppf) Cl₂ (19.2 g, 26.2 mmol) and CuI (2.00 g, 10.4 mmol) under N₂, then the mixture was stirred at 80° C., for 16 hrs. On completion, the mixture was filtered and added EtOAc (5.00 L), and washed with water (2.00 L×3). The organic layer was washed with brine (2.00 L), dried over Na₂SO₄, filtered and concentrated to give a residue. The residue was purified by column chromatography (SiO₂, Petroleum ether: Ethyl acetate=50/1 to 5/1). The combined organic layers were concentrated under reduced pressure to give the title compound (150 g, 88% yield) as a gray solid. LC-MS (ESI⁺) m/z 248.2 (M-56)⁺: ¹HNMR (400 MHz, d₆-DMSO). δ: 0.22 (s, 9H), 1.24-1.43 (m, 9H), 4.12 (br, d, J=6.11 Hz, 2H), 7.22 (d, J=8.07 Hz, 2H), 7.38-7.47 (m, 3H).

Step 2—Tert-butyl 4-ethynylbenzylcarbamate. To a solution of tert-butyl 4-((trimethylsilyl)ethynyl)benzylcarbamate (150 g, 459 mmol) in MeOH (1.10 L) was added K₂CO₃ (127 g, 918 mmol), and the mixture was stirred at 25° C., for 2 hrs. The mixture was filtered and concentrated under reduced pressure to give the title compound (180 g) as a gray solid. ¹HNMR (400 MHZ, CDCl₃). δ 1.45 (br, s, 9H), 3.33 (s, 1H), 4.25-4.39 (m, 2H), 7.23 (br, d, J=6.90 Hz, 2H), 7.45 (br, d, J=7.40 Hz, 2H).

Step 3—(4-Ethynylphenyl)methanamine. To a solution of tert-butyl 4-ethynylbenzylcarbamate (107 g, 462 mmol) in DCM (1.00 L) was added HCl/dioxane (4 M, 273 mL), then the mixture was stirred at 25° C., for 12 hrs. The mixture was concentrated under reduced pressure to give the title compound (140 g. 3 HCl) as a yellow solid. ¹HNMR (400 MHZ, DMSO) δ 4.02 (br, s, 2H), 4.25 (br, s, 1H), 7.43-7.59 (m, 4H), 8.65 (br, s, 3H).

Step 4—(2S,4R)-tert-butyl 2-((4-ethynylbenzyl)carbamoyl)-4-hydroxypyrrolidine-1-carboxylate. To a solution of (4-ethynylphenyl)methanamine (132 g, 548 mmol, 3HCl) in DMSO (1.30 L) was added DIPEA (189 g, 1.46 mol), (2S,4R)-1-(tert-butoxycarbonyl)-4-hydroxypyrrolidine-2-carboxylic acid (84.6 g, 365 mmol) and HOAt (60.0 g, 440 mmol) and EDCI (84.2 g, 439 mmol), then the mixture was stirred at 25° C., for 3 hrs. The mixture was diluted with water (3.00 L) and extracted with EtOAc (2.00 L×3), and the combined organic layers were washed with H₂O (1.00 L×3) and brine (2.00 L), and dried over Na₂SO₄, filtered and concentrated under reduced pressure to give a residue. The residue was purified by column chromatography (SiO₂, Petroleum ether:Ethyl acetate=10/1 to 0/1) and concentrated under reduced pressure to give the title compound (118 g, 88% yield) as a yellow oil. LC-MS (ESI) m/z 245.1 (M-100).

Step 5—(2S,4R)—N-(4-ethynylbenzyl)-4-hydroxypyrrolidine-2-carboxamide. To a solution of (2S,4R)-tert-butyl 2-((4-ethynylbenzyl)carbamoyl)-4-hydroxypyrrolidine-1-carboxylate (115 g, 307 mmol) in DCM (1.20 L) was added HCl/dioxane (4 M, 140 mL) at 0° C., then the mixture was stirred at 25° C., for 12 hrs. On completion, the mixture was filtered and the crude product was diluted with MeOH (900 mL). Then alkaline resin (450 g), was added and the mixture was stirred at 25° C., for 3 hrs. The mixture was filtered and concentrated under reduced pressure to give the title compound as a yellow solid. LC-MS (ESI) m/z 245.1 (M+1); ¹HNMR (400 MHZ, d₆-DMSO). δ 1.74-1.85 (m, 1H), 2.15 (br, dd, J=13.08, 7.58 Hz, 1H), 2.93 (br, d, J=11.86 Hz, 1H), 3.09 (dd, J=11.86, 4.03 Hz, 1H), 4.08 (dd, J=9.35, 7.89 Hz, 1H), 4.16 (s, 1H), 4.32 (br, d, J=5.99 Hz, 3H), 5.17 (br, s, 1H), 7.26 (d, J=8.07 Hz, 2H), 7.43 (d, J=7.95 Hz, 2H), 8.88 (br, t, J=5.75 Hz, 1H).

### (2S,4R)-1-((S)-2-amino-3,3-dimethylbutanoyl)-N-(4-ethynylbenzyl)-4-hydroxypyrrolidine-2-carboxamide (Intermediate EO)

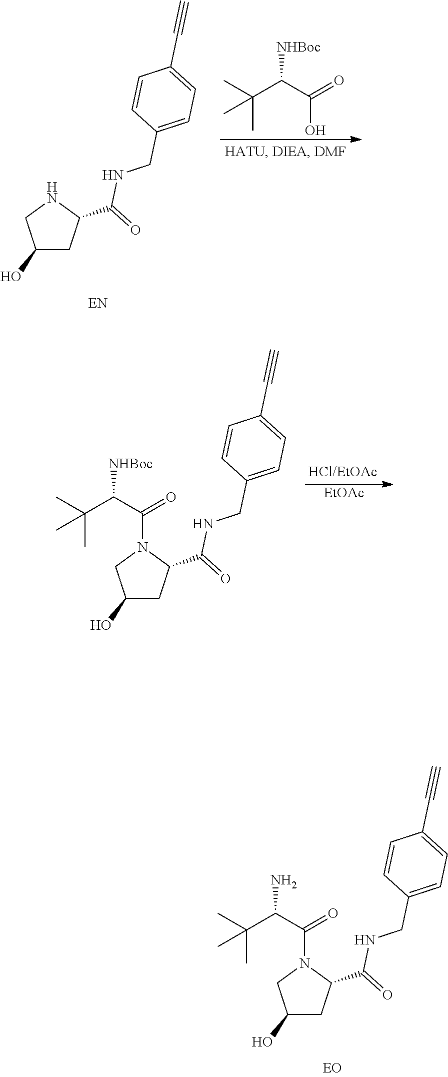

Step 1—Tert-butyl ((S)-1-((2S,4R)-2-((4-ethynylbenzyl)carbamoyl)-4-hydroxypyrrolidin-1-yl)-3,3-dimethyl-1-oxobutan-2-yl) carbamate. To a solution of(S)-2-((tert-butoxycarbonyl)amino)-3,3-dimethylbutanoic acid (1.89 g, 8.19 mmol, CAS #62965-35-9), DIEA (5.29 g, 40.9 mmol, 7.13 mL) and HATU (3.74 g, 9.82 mmol) in DMF (20 mL) was added (2S,4R)—N-(4-ethynylbenzyl)-4-hydroxypyrrolidine-2-carboxamide (2 g, 8.19 mmol, Intermediate EN) at 0° C. The mixture was stirred at 25° C., for 12 h. On completion, the reaction mixture was quenched with water (20 mL) at 20° C., extracted with EtOAc (10 mL×2). The combined organic layers were washed with brine (10 mL×2), dried over Na₂SO₄, filtered and concentrated under reduced pressure to give a residue. The residue was purified by column chromatography (SiO₂, Petroleum ether/Ethyl acetate=3/1 to 1/3) to give the title compound (2.95 g, 59% yield) as a brown liquid. ¹H NMR (400 MHZ, DMSO-d₆) δ=8.56 (br s, 1H), 7.41-7.27 (m, 4H), 6.45 (br d, J=8.8 Hz, 1H), 5.13 (br s, 1H), 4.47-4.31 (m, 3H), 4.25-4.09 (m, 3H), 3.62 (br d, J=10.8 Hz, 2H), 2.08-2.00 (m, 1H), 1.87 (br s, 1H), 1.38 (br s, 9H), 0.93 (br s, 9H).

Step 2—(2S,4R)-1-((S)-2-amino-3,3-dimethylbutanoyl)-N-(4-ethynylbenzyl)-4-hydroxypyrrolidine-2-carboxamide. To a solution of tert-butyl ((S)-1-((2S,4R)-2-((4-ethynylbenzyl)carbamoyl)-4-hydroxypyrrolidin-1-yl)-3,3-dimethyl-1-oxobutan-2-yl) carbamate (1 g, 2.19 mmol) in EtOAc (10 mL) was added HCl/EtOAc (4 M, 10 ml). The mixture was stirred at 25° C. for 12 h. On completion, the reaction mixture was concentrated under reduced pressure to give the title compound (600 mg, 60% yield) as a white solid. ¹H NMR (400 MHZ, DMSO-d₆) δ=8.75 (br t,]=6.0 Hz, 1H), 7.40-7.34 (m, 2H), 7.32-7.27 (m, 2H), 4.52 (br t,]=8.4 Hz, 1H), 4.44-4.32 (m, 2H), 4.23-4.11 (m, 2H), 3.89 (br d, J=4.8 Hz, 1H), 3.77 (br d, J=11.2 Hz, 1H), 3.54 (br dd, J=4.0, 10.8 Hz, 1H), 2.14-2.04 (m, 1H), 1.92-1.82 (m, 2H), 1.40-1.20 (m, 1H), 1.02 (s, 9H), 0.93-0.82 (m, 1H).

### Tert-butyl ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl) carbamate (Intermediate EP)

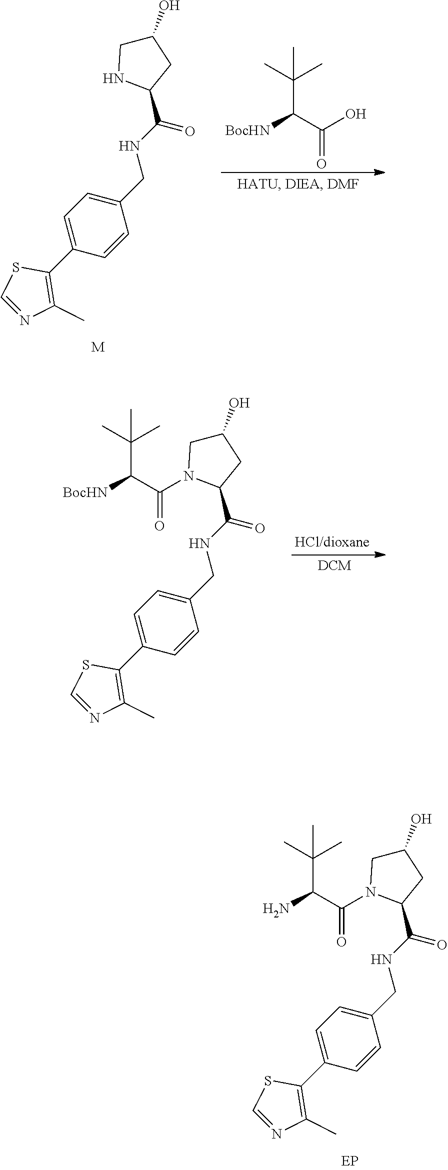

Step 1—((S)-1-((2S,4R)-4-hydroxy-2-((4-(4-methylthiazol-5-Tert-butyl yl)benzyl)carbamoyl)pyrrolidin-1-yl)-3,3-dimethyl-1-oxobutan-2-yl) carbamate. To a solution of (2S,4R)-4-hydroxy-N-[[4-(4-methylthiazol-5-yl)phenyl]methyl]pyrrolidine-2-carboxamide (5 g, 15.7 mmol, Intermediate M), (2S)-2-(tert-butoxycarbonylamino)-3,3-dimethyl-butanoic acid (3.64 g, 15.7 mmol, CAS #62965-35-9) in DMF (50 mL) was added HATU (7.19 g, 18.9 mmol) and DIEA (10.1 g, 78.7 mmol, 13.7 mL). The mixture was stirred at 25° C., for 12 h. On completion, the reaction mixture was diluted with water (100 mL) and extracted with EtOAc (150 mL×3). The combined organic layers were washed with brine (300 mL×2), dried over Na₂SO₄ and evaporated. The residue was purified by column chromatography (SiO₂, Petroleum ether:Ethyl acetate=5:1 to Petroleum ether:Ethyl acetate=0:1) to give the title compound (8 g, 96% yield) as a yellow oil. LC-MS (ESI⁺) m/z 531.5 (M+H)⁺.

Step 2—Tert-butyl ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl) carbamate. To a solution of tert-butyl N-[(1S)-1-[(2S,4R)-4-hydroxy-2-[4-(4-methylthiazol-5-yl)phenyl]methylcarbamoyl]pyrrolidine-1-carbonyl]-2,2-dimethyl-propyl]carbamate (7.9 g, 14.8 mmol) in DCM (80 mL) was added HCl/dioxane (4 M, 3.72 mL). The mixture was stirred at 25° C., for 1 h. On completion, the mixture was concentrated in vacuo to give the title compound (6 g, 13.94 mmol, 94% yield) as a brown solid. LC-MS (ESI⁺) m/z 431.2 (M+H)⁺.

### Ethyl 3-(5-sulfamoylpyridin-2-yl) propanoate (Intermediate EQ)

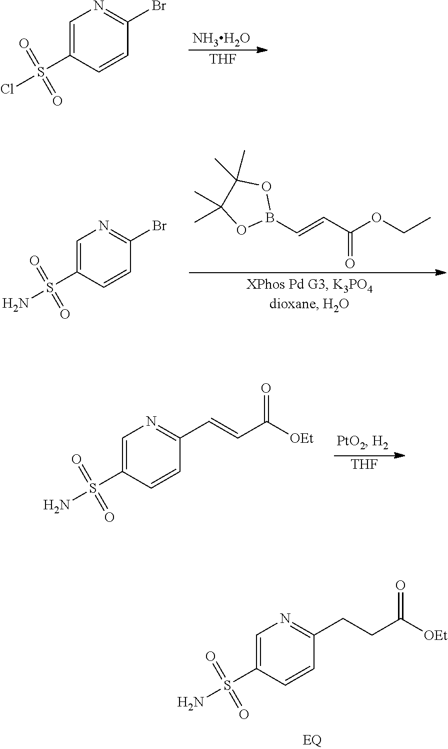

Step 1—6-Bromopyridine-3-sulfonamide. To a solution of 6-bromopyridine-3-sulfonyl chloride (2 g, 7.80 mmol, CAS #886371-20-6) in THF (12 mL) was added NH₃·H₂O (10.9 g, 77.9 mmol, 12.0 mL, 25% solution) at 0° C. The mixture was stirred at 25° C., for 2 h. On completion, the mixture was concentrated in vacuo. The mixture was diluted with H₂O (5 mL), and extracted with DCM (5 mL×3). The combined organic layer was then washed with brine (10 ml×2), dried over anhydrous Na₂SO₄, filtered and concentrated under reduced pressure to give a residue. The residue was purified by column chromatography (SiO₂, Petroleum ether:Ethyl acetate=10:1 to Petroleum ether:Ethyl acetate=0:1) to give the title compound (1.3 g, 70% yield) as a white solid. LC-MS (ESI⁺) m/z 236.9 (M+H)⁺.

Step 2—(E)-ethyl 3-(5-sulfamoylpyridin-2-yl) acrylate. To a solution of 6-bromopyridine-3-sulfonamide (500 mg, 2.11 mmol) and ethyl (E)-3-(4,4,5,5-tetramethyl-1,3,2-dioxaborolan-2-yl) prop-2-enoate (715 mg, 3.16 mmol) in dioxane (3 mL) and H₂O (3 mL) was added XPhos Pd G3 (89 mg, 105 umol) and K₃PO₄ (1.34 g, 6.33 mmol). The mixture was then stirred at 80° C., for 2 h. On completion, the mixture was concentrated in vacuo. Then H₂O (5 mL) was added, and the mixture was extracted with DCM (5 mL×3). The combined organic layer was washed with brine (10 ml×2), dried over anhydrous Na₂SO₄, filtered and concentrated under reduced pressure to give a residue. The residue was purified by column chromatography (SiO₂, Petroleum ether:Ethyl acetate=5:1 to Petroleum ether:Ethyl acetate=0:1) to give the title compound (240 mg, 44% yield) as a white solid. LC-MS (ESI⁺) m/z 257.1 (M+H)⁺.

Step 3—Ethyl 3-(5-sulfamoylpyridin-2-yl) propanoate. To a solution of Pt02 (212 mg, 936 umol) in THF (2 mL) was added ethyl (E)-3-(5-sulfamoyl-2-pyridyl) prop-2-enoate (240 mg, 936 umol) under N₂ atmosphere. The suspension was degassed and purged with H₂ for 3 times. Then the mixture was stirred under H₂ (15 Psi) at 25° C., for 12 h. On completion, the reaction mixture was filtered and the filtrate was concentrated under reduced pressure to give the title compound (220 mg) as a yellow solid. LC-MS (ESI⁺) m/z 259.3 (M+H)⁺.

### 3-(5-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)pyridin-2-yl) propanoic acid (Intermediate ER)

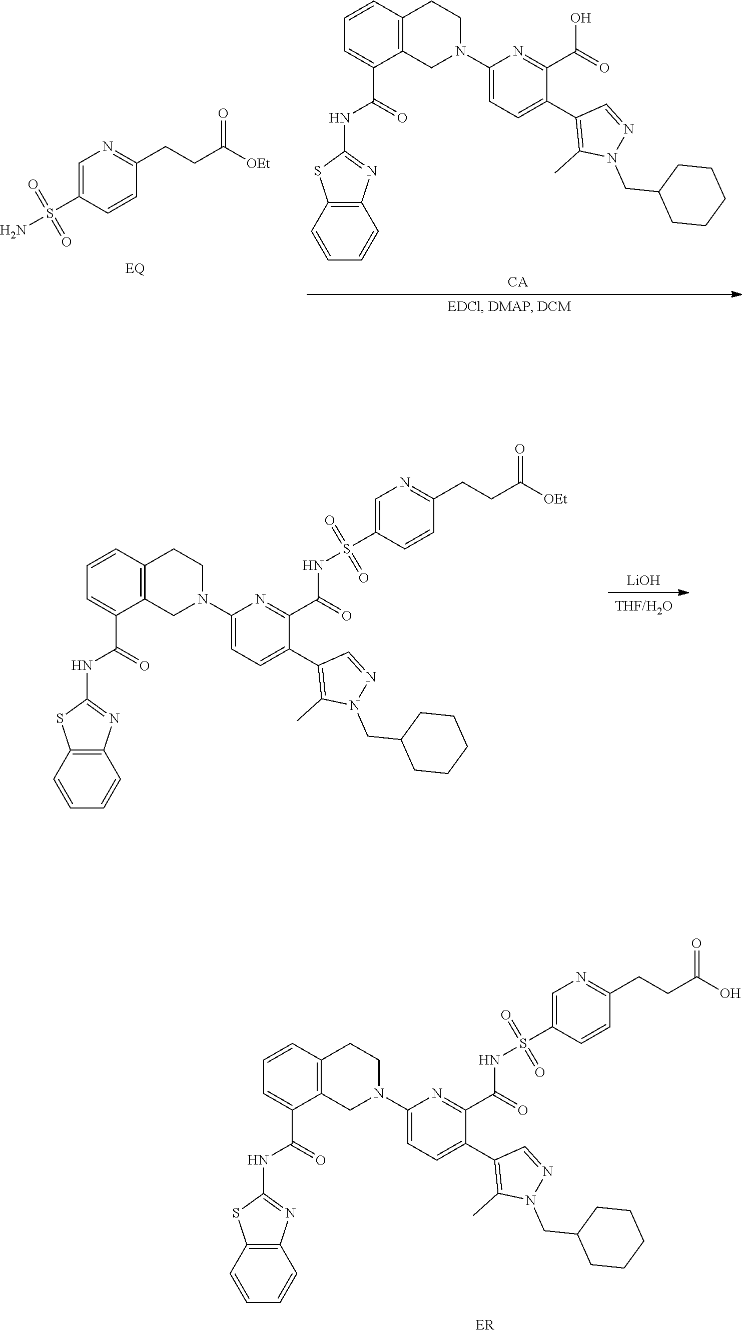 [SMILES: CCOC(=O)CCc1ccc(S(N)(=O)=O)cn1 ; Cc1c(-c2ccc(N3CCc4cccc(C(=O)Nc5nc6ccccc6s5)c4C3)nc2C(=O)O)cnn1CC1CCCCC1 ; CCOC(=O)CCc1ccc(S(=O)(=O)NC(=O)c2nc(N3CCc4cccc(C(=O)Nc5nc6ccccc6s5)c4C3)ccc2-c2cnn(CC3CCCCC3)c2C)cn1 ; "LiOH" ; Cc1c(-c2ccc(N3CCc4cccc(C(=O)Nc5nc6ccccc6s5)c4C3)nc2C(=O)NS(=O)(=O)c2ccc(CCC(=O)O)nc2)cnn1CC1CCCCC1 ; "ER"]

Step 1—Ethyl 3-(5-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)pyridin-2-yl) propanoate. To a solution of ethyl 3-(5-sulfamoyl-2-pyridyl) propanoate (100 mg, 387 umol, Intermediate EQ), 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (234 mg, 387 umol, Intermediate CA) in DCM (2 mL) was added EDCI (148 mg, 774 umol) and DMAP (118 mg, 967 umol). The mixture was stirred at 40° C., for 12 h. On completion, the mixture was quenched with H₂O (4 mL), and extracted with DCM (5 mL×3). The combined organic was then washed with brine (10 ml×2), dried over anhydrous Na₂SO₄, filtered and concentrated under reduced pressure to give a residue. The residue was purified by column chromatography (SiO₂, Petroleum ether:Ethyl acetate=5:1 to Dichloromethane:Methanol=10:1) to give the title compound (250 mg, 76% yield) as a white solid. LC-MS (ESI⁺) m/z 847.3 (M+H)⁺.

Step 2—3-(5-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)pyridin-2-yl) propanoic acid. To a solution of ethyl 3-[5-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]-2-pyridyl]propanoate (150 mg, 177 umol) in THF (1 mL) and H₂O (1 mL) was added LiOH·H₂O (37.1 mg, 885 umol). The mixture was stirred at 25° C., for 12 h. On completion, the mixture was adjusted pH to acidity (4˜5) and concentrated in vacuo. The mixture was quenched with H₂O (4 mL), then extracted with EtOAc mL (5 mL×3). The combined organic layer was then washed with brine (10 mL×2), dried over anhydrous Na₂SO₄, filtered and concentrated under reduced pressure to give the title compound (125 mg, 86% yield) as a white solid. LC-MS (ESI⁺) m/z 819.4 (M+H)⁺.

### (S)-methyl 1-(4-sulfamoylphenyl) pyrrolidine-3-carboxylate (Intermediate ES)

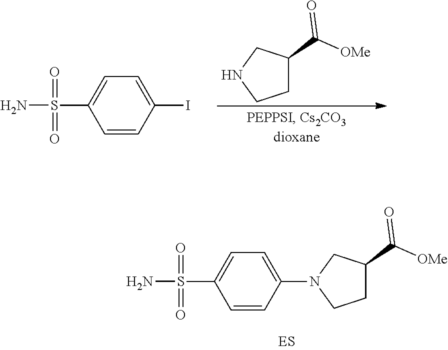 [SMILES: NS(=O)(=O)c1ccc(I)cc1 ; COC(=O)[C@H]1CCNC1 ; COC(=O)[C@H]1CCN(c2ccc(S(N)(=O)=O)cc2)C1]

To a solution of 4-iodobenzenesulfonamide (500 mg, 1.77 mmol, CAS #825-86-5) and(S)-methyl pyrrolidine-3-carboxylate (878 mg, 5.30 mmol, HCl, CAS #216311-60-3) in dioxane (20 mL) was added Cs₂CO₃ (2.30 g, 7.06 mmol) and Pd-PEPPSI-IHeptCl (172 mg, 177 umol). The mixture was stirred at 100° C., for 2 h under N₂ atmosphere. On completion, the reaction mixture was quenched with water (30 mL) at 20° C., and extracted with EtOAc (40 mL×3). The combined organic layers were washed with brine (80 mL×2), dried over Na₂SO₄, filtered and concentrated under reduced pressure to give a residue. The crude product was purified by reversed-phase HPLC (0.1% FA condition) to give the title compound (320 mg, 64% yield) as a white solid. LC-MS (ESI⁺) m/z 285.0 (M+H)⁺.

### Methyl(S)-1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylic acid (Intermediate ET)

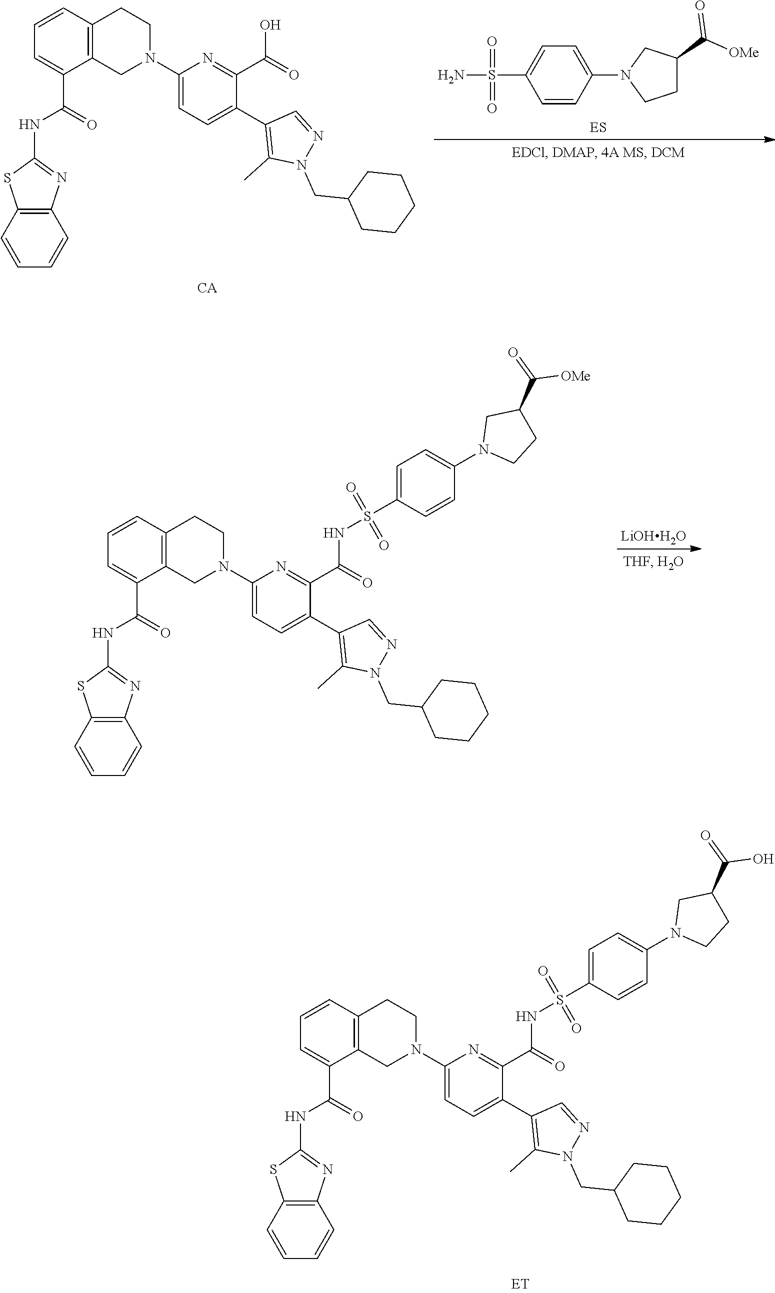

Step 1—(S)-methyl 1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylate. To a solution of methyl(S)-methyl 1-(4-sulfamoylphenyl) pyrrolidine-3-carboxylate (320 mg, 1.13 mmol, Intermediate ES), 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid (683 mg, 1.13 mmol, Intermediate CA) and 4A molecular sieves (480 mg) in DCM (5 mL) was added DMAP (550 mg, 4.50 mmol) and EDCI (432 mg, 2.25 mmol). The mixture was stirred at 25° C., for 12 h. On completion, the reaction mixture was quenched with water (10 mL) at 20° C., and extracted with DCM (8 mL×2). The combined organic layers were washed with brine (10 mL×2), dried over Na₂SO₄, filtered and concentrated under reduced pressure to give a residue. The residue was purified by column chromatography (SiO₂, Petroleum ether/Ethyl acetate=3/1 to 1/2) to give the title compound (500 mg, 51% yield) as a white solid. LC-MS (ESI⁺) m/z 873.5 (M+H)+.

Step 2—Methyl(S)-1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylic acid. To a solution of(S)-methyl 1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylate (500 mg, 573 umol) in THF (3.75 mL) and H₂O (1.25 mL) was added LiOH·H₂O (120 mg, 2.86 mmol). The mixture was stirred at 25° C., for 12 h. On completion, the reaction mixture was quenched with water (5 mL) at 20° C., and extracted with DCM (8 mL×2). Then the aqueous phase was adjusted pH to 3˜4 and extracted with DCM (8 mL×3). The combined organic layer was washed with brine (10 mL×2), dried over Na₂SO₄, filtered and concentrated under reduced pressure to give the title compound (360 mg, 73% yield) as a yellow solid. ¹H NMR (400 MHz, DMSO-d₆) δ=12.96 (td, J=2.8, 5.4 Hz, 1H), 11.73 (s, 1H), 8.03 (d, J=8.0 Hz, 1H), 7.80 (d, J=8.0 Hz, 1H), 7.64 (d, J=7.6 Hz, 1H), 7.62 (s, 1H), 7.60 (s, 1H), 7.50-7.46 (m, 1H), 7.46-7.44 (m, 1H), 7.43 (s, 1H), 7.40-7.32 (m, 2H), 7.13 (s, 1H), 6.98 (d, J=8.8 Hz, 1H), 6.53 (d, J=9.2 Hz, 2H), 4.96 (s, 2H), 3.92 (br t, J=6.0 Hz, 2H), 3.73 (d, J=7.2 Hz, 2H), 3.54-3.48 (m, 1H), 3.47-3.42 (m, 1H), 3.33-3.26 (m, 2H), 3.25-3.16 (m, 1H), 3.01 (br t, J=5.6 Hz, 2H), 2.29-2.19 (m, 1H), 2.18-2.12 (m, 1H), 1.83 (s, 3H), 1.78-1.71 (m, 1H), 1.70-1.59 (m, 3H), 1.50 (br d, J=12 Hz, 2H), 1.28-1.02 (m, 4H), 0.99-0.85 (m, 2H).

### 1-(cyclopentylmethyl)-5-methyl-4-(4,4,5,5-tetramethyl-1,3,2-dioxaborolan-2-yl)-1H-pyrazole (Intermediate EU)

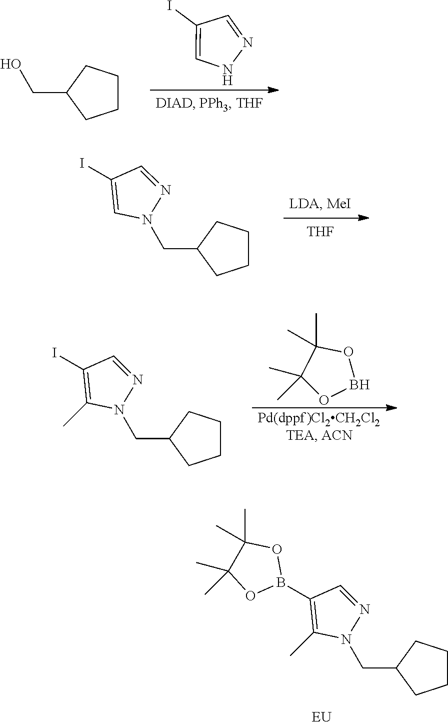

Step 1 1-(Cyclopentylmethyl)-4-iodo-1H-pyrazole. To a solution of 4-iodo-1H-pyrazole (21.3 g, 109.8 mmol, CAS #3469-69-0) and cyclopentylmethanol (10 g, 99.8 mmol, 10.8 mL, CAS #3637-61-4) in THF (120 mL) was added DIAD (30.3 g, 149.8 mmol, 29.1 mL) and PPh₃ (39.3 g, 149.8 mmol) in THF (50.0 mL) dropwise at 0° C., under N₂. The mixture was then stirred at 25° C., for 12 h. On completion, the mixture was quenched with H₂O (150 mL) and extracted with EtOAc (200 mL×3). The combined organic layers was washed with brine (150 mL×3) and dried over Na₂SO₄ and concentrated in vacuo. The residue was purified by silica gel column chromatography (Petroleum ether:Ethyl acetate=1:0˜30:1) to afford the title compound (18.1 g, 66% yield) as a white solid. ¹H NMR (400 MHZ, DMSO-d₆) δ 7.92 (s, 1H), 7.49 (s, 1H), 4.01 (d, J=7.6 Hz, 2H), 2.32 (td, J=7.2, 14.6 Hz, 1H), 1.59-1.53 (m, 4H), 1.52-1.45 (m, 2H), 1.23-1.16 (m, 2H).

Step 2—1-(Cyclopentylmethyl)-4-iodo-5-methyl-1H-pyrazole. To a solution of 1-(cyclopentylmethyl)-4-iodo-1H-pyrazole (18.1 g, 65.6 mmol) in THF (150 mL) was added LDA (2 M, 52.4 mL) at −70° C., and the mixture was stirred at −70° C., for 1 h. Then, Mel (14.0 g, 98.3 mmol, 6.12 mL) in THF (20 mL) was added in dropwise and the mixture was stirred at 70 to 25° C., over 12 h. On completion, the mixture was quenched with H₂O (100 mL) and extracted with EtOAc (300 mL×3). The combined organic layers was washed with brine (100 mL×3) and dried over Na₂SO₄ and concentrated in vacuo. The residue was purified by silica gel column chromatography (Petroleum ether:Ethyl acetate=30:1˜20:1) to afford the title compound (17.0 g, 89% yield) as a white solid. ¹H NMR (400 MHZ, DMSO-d₆) δ 7.42 (s, 1H), 4.00 (d, J=7.6 Hz, 2H), 2.36-2.28 (m, 1H), 2.25 (s, 3H), 1.64-1.53 (m, 4H), 1.53-1.43 (m, 2H), 1.27-1.16 (m, 2H).

Step 3—1-(cyclopentylmethyl)-5-methyl-4-(4,4,5,5-tetramethyl-1,3,2-dioxaborolan-2-yl)-1H-pyrazole. To a solution of 1-(cyclopentylmethyl)-4-iodo-5-methyl-1H-pyrazole (17.0 g, 58.5 mmol), 4,4,5,5-tetramethyl-1,3,2-dioxaborolane (22.5 g, 175.6 mmol, 25.5 mL) and TEA (17.8 g, 175.6 mmol, 24.4 mL) in ACN (180 mL) was added Pd (dppf) Cl₂·CH₂Cl₂ (3.35 g, 4.10 mmol). Then the mixture was stirred at 80° C., for 4 h under N₂ atmosphere. On completion, the reaction mixture was quenched with H₂O (50 mL) and extracted with EtOAc (80 mL×2). The combined organic layers were dried over Na₂SO₄ and evaporated. The residue was purified by silica gel column chromatography (PE:EA=1:0˜30:1) to afford the title compound (11.0 g, 65% yield) as a white solid. ¹H NMR (400 MHZ, DMSO-d₆) δ 7.44 (s, 1H), 3.92 (d, J=7.6 Hz, 2H), 2.37 (s, 3H), 2.34-2.28 (m, 1H), 1.65-1.52 (m, 6H), 1.52-1.46 (m, 2H), 1.24 (s, 12H).

### Methyl 6-(8-(benzo[d]thiazol-2-ylcarbamoyl)-3,4-dihydroisoquinolin-2 (1H)-yl)-3-bromopicolinate (Intermediate EV)

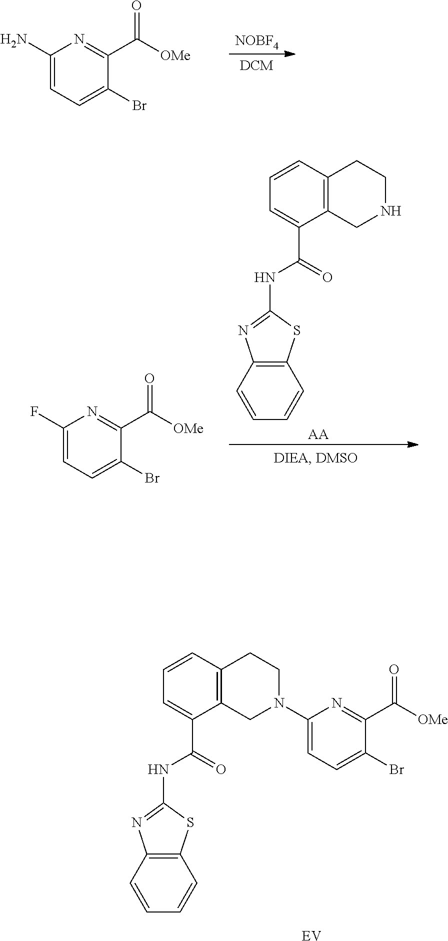

Step 1—Methyl 3-bromo-6-fluoropicolinate. To a solution of nitridooxonium tetrafluoroborate (65.7 g, 562 mmol, CAS #14635-75-7) in DCM (1000 mL) was added a solution of methyl 6-amino-3-bromo-pyridine-2-carboxylate (100 g, 432 mmol, CAS #178876-83-0) in DCM (200 mL) dropwise at 0° C. under N₂. Then the mixture was stirred at 25° C., for 12 h. On completion, the reaction mixture was quenched with NaHCO₃ (100 mL) and extracted with DCM (300 mL×3). The combined organic layers were washed with brine (100 mL×3), dried over Na₂SO₄ and evaporated. The residue was purified by silica gel column chromatography (PE:EA=20:1˜1:1) to give a title compound (82.1 g, 58% yield) as a yellow solid. ¹H NMR (400 MHz, DMSO-d₆) δ=8.42 (dd, J=7.2, 8.4 Hz, 1H), 7.42 (dd, J=3.2, 8.8 Hz, 1H), 3.92-3.90 (m, 3H).

Step 2—Methyl 6-(8-(benzo[d]thiazol-2-ylcarbamoyl)-3,4-dihydroisoquinolin-2 (1H)-yl)-3-bromopicolinate. To a solution of N-(1,3-benzothiazol-2-yl)-1,2,3,4-tetrahydroisoquinoline-8-carboxamide (10 g, 28.91 mmol, HCl, Intermediate AA), methyl 3-bromo-6-fluoro-pyridine-2-carboxylate (10.3 g, 31.8 mmol) in DMSO (60 mL) was added DIEA (18.69 g, 144.57 mmol, 25.18 mL), then the mixture was stirred at 60° C., for 12 h. On completion, the reaction mixture was poured into water (60 mL), and then extracted with EtOAc (10 mL×2). The combined organic layers were washed with brine (10 mL×3), dried over Na₂SO₄, filtered and concentrated under reduced pressure to give a residue. The crude product was triturated with PE:EA=10:1 at 25° C., for 30 min to give a title compound (12.9 g, 24.65 mmol, 85% yield) as a white solid. LC-MS (ESI⁺) m/z 523.1+.

### 6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinic acid (Intermediate EW)

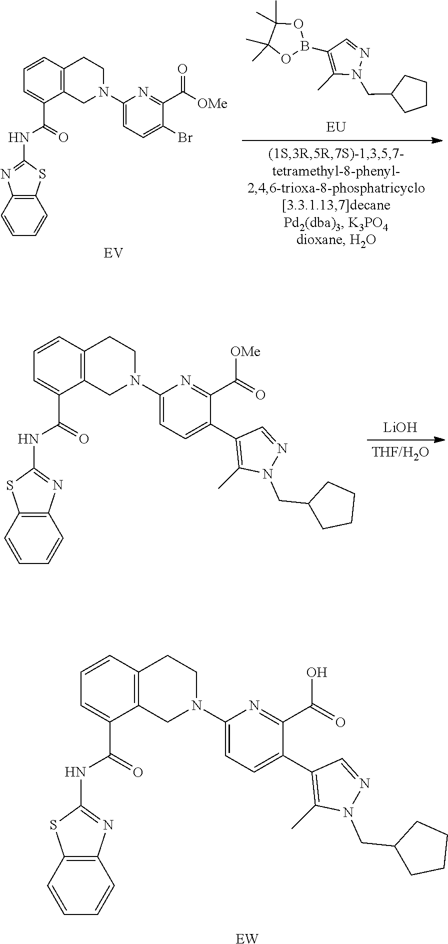

Step 1—methyl 6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinate. To a solution of methyl 6-(8-(benzo[d]thiazol-2-ylcarbamoyl)-3,4-dihydroisoquinolin-2 (1H)-yl)-3-bromopicolinate (500.0 mg, 955.3 umol, Intermediate EV) and 1-(cyclopentylmethyl)-5-methyl-4-(4,4,5,5-tetramethyl-1,3,2-dioxaborolan-2-yl)-1H-pyrazole (471.3 mg, 1.62 mmol, Intermediate EU) in dioxane (5 mL) and H₂O (5 mL) was added Pd₂(dba); (87.5 mg, 95.5 umol), K₃PO₄ (608.3 mg, 2.87 mmol) and (1S,3R,5R,7S)-1,3,5,7-tetramethyl-8-phenyl-2,4,6-trioxa-8-phosphatricyclo[3,3,1,1^{3,7}]decane (111.7 mg, 382.1 umol). The mixture was stirred at 100° C., for 5 h under N₂. On completion, the reaction mixture was quenched with H₂O (5 mL) at 25° C., and extracted with EtOAc (10 mL×3). The combined organic layers were washed with H₂O (5 mL×3), dried over Na₂SO₄ filtered and concentrated under reduced pressure to give a residue. The residue was purified by column chromatography (SiO₂, Petroleum ether/Ethyl acetate=10/1 to 0/1) to afford the title compound (303.0 mg, 52% yield) as an off-white solid. ¹H NMR (400 MHZ, DMSO-d₆) δ 12.87 (br s, 1H), 8.04 (d, J=8.0 Hz, 1H), 7.79 (d, J=8.4 Hz, 1H), 7.60 (br d, J=7.6 Hz, 1H), 7.52 (d, J=8.8 Hz, 1H), 7.49-7.41 (m, 2H), 7.39-7.32 (m, 2H), 7.20 (s, 1H), 6.97 (d, J=9.2 Hz, 1H), 4.94 (s, 2H), 3.93 (d, J=7.6 Hz, 2H), 3.85 (br t, J=5.8 Hz, 2H), 3.52 (s, 2H), 3.01 (br t, J=5.8 Hz, 2H), 2.33 (td, J=7.2, 14.6 Hz, 1H), 2.09 (s, 3H), 1.63-1.44 (m, 6H), 1.40 (d, J=9.2 Hz, 1H), 1.27-1.21 (m, 2H).

Step 2—6-(8-(Benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinic acid. To a solution of methyl 6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinate (303.0 mg, 499.4 umol) in THF (3 mL) and H₂O (1 mL) was added LiOH·H₂O (104.8 mg, 2.50 mmol). The mixture was then stirred at 25° C., for 12 h. On completion, the reaction mixture was diluted with water (2 mL) and the combined organic layers were extracted with DCM (3 mL). The water phase was adjusted pH to 5˜6 by addition 2 M HCl. The water phase was then filtered and the filter cake was dried under reduced pressure to afford the title compound (291 mg, 96% yield) as a white solid. LC-MS (ESI⁺) m/z 593.0 (M+H)⁺.

### 1-(4-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)phenyl)piperidine-4-carboxylic acid (Intermediate EX)

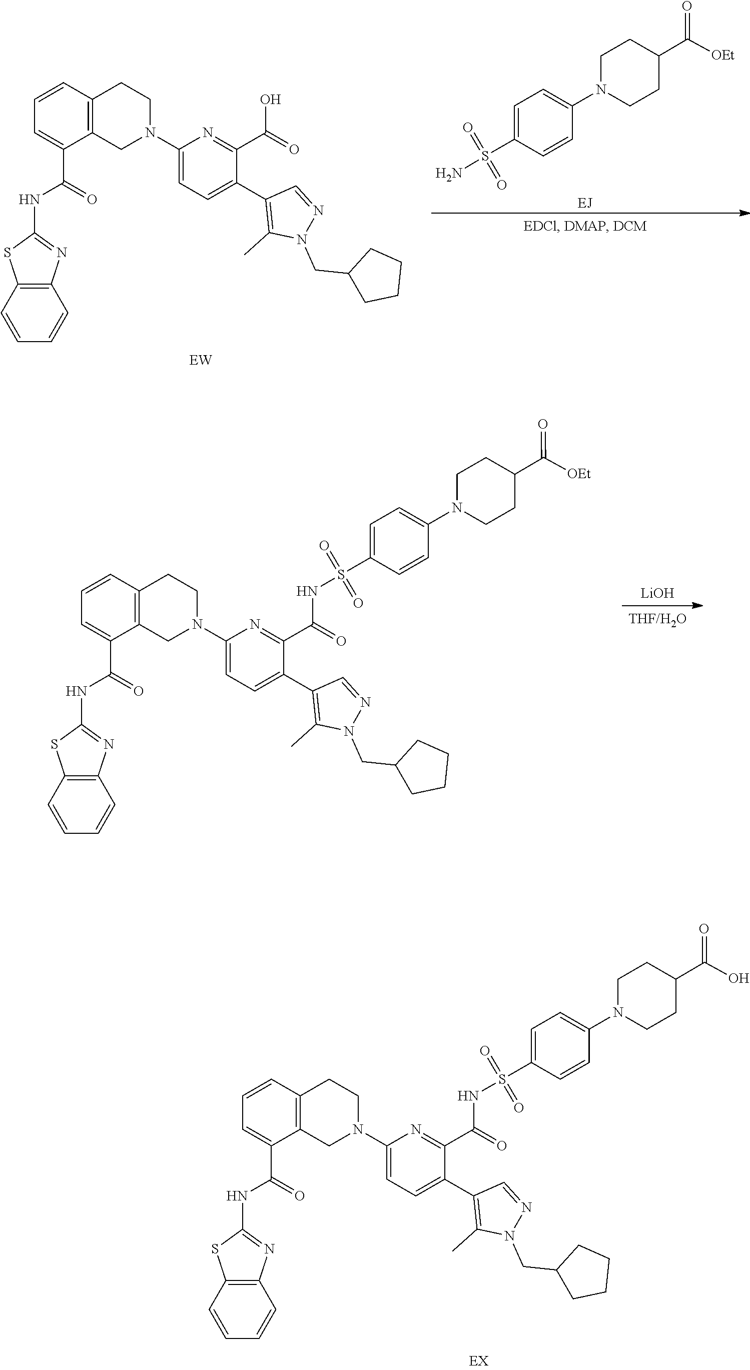 [SMILES: Cc1c(-c2ccc(N3CCc4cccc(C(=O)Nc5nc6ccccc6s5)c4C3)nc2C(=O)O)cnn1CC1CCCC1 ; CCOC(=O)C1CCN(c2ccc(S(N)(=O)=O)cc2)CC1 ; "EJ" ; CCOC(=O)C1CCN(c2ccc(S(=O)(=O)NC(=O)c3nc(N4CCc5cccc(C(=O)Nc6nc7ccccc7s6)c5C4)ccc3-c3cnn(CC4CCCC4)c3C)cc2)CC1 ; "LiOH" ; Cc1c(-c2ccc(N3CCc4cccc(C(=O)Nc5nc6ccccc6s5)c4C3)nc2C(=O)NS(=O)(=O)c2ccc(N3CCC(C(=O)O)CC3)cc2)cnn1CC1CCCC1]

Step 1—Ethyl 1-(4-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)phenyl)piperidine-4-carboxylate. To a solution of 6-[8-(1,3-benzothiazol-2-ylcarbamoyl)-3,4-dihydro-1H-isoquinolin-2-yl]-3-[1-(cyclopentylmethyl)-5-methylpyrazol-4-yl]pyridine-2-carboxylic acid (250 mg, 421 umol, Intermediate EW), ethyl 1-(4-sulfamoylphenyl)piperidine-4-carboxylate (145.0 mg, 464 umol, Intermediate EJ) in DCM (4 mL) was added EDCI (161.7 mg, 843.6 umol) and DMAP (128.8 mg, 1.1 mmol). The mixture was then stirred at 40° C., for 2 h. On completion, the reaction mixture was quenched with H₂O (5 mL) and extracted with DCM (20 mL×2). The combined organic layers were dried over Na₂SO₄ and evaporated. The crude product was purified by reversed-phase HPLC (ACN/0.1% FA=70%) to afford the title compound (180.0 mg, 48.11% yield) as an orange solid. LC-MS (ESI⁺) m/z 887.2 (M+H)⁺.

Step 2—1-(4-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)phenyl)piperidine-4-carboxylic acid. To a solution of ethyl 1-(4-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)phenyl)piperidine-4-carboxylate (56.0 mg, 63.1 umol) in THF (1 mL) and H₂O (1 mL) was added LiOH·H₂O (13.3 mg, 315.6 umol). The mixture was then stirred at 25° C., for 2 h. On completion, the reaction mixture was diluted with H₂O (1 mL) and the combined organic layers were extracted with DCM (2 mL). The water phase was adjusted pH to 3˜4 by addition 2 M HCl. The aqueous phase was then filtered and the filter cake was dried under reduced pressure to give the title compound (50.0 mg, 92% yield) as an orange solid. LC-MS (ESI⁺) m z 859.4 (M+H)⁺.

### 5-Methyl-1-neopentyl-4-(4,4,5,5-tetramethyl-1,3,2-dioxaborolan-2-yl)-1H-pyrazole (Intermediate EY)

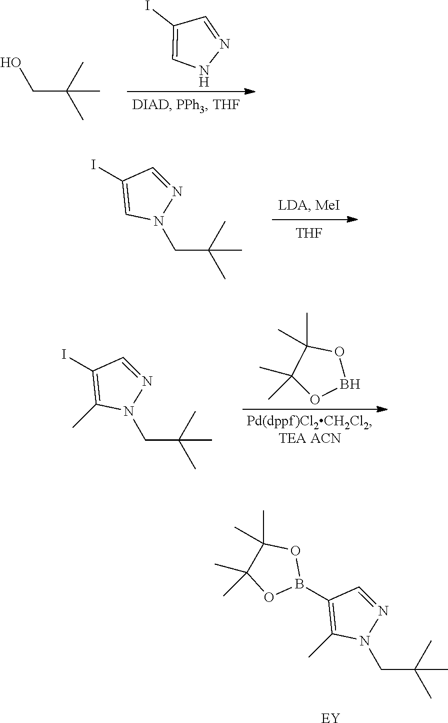

Step 1—4-Iodo-1-neopentyl-1H-pyrazole. To a solution of 2,2-dimethylpropan-1-ol (15 g, 170 mmol, CAS #75-84-3) and 4-iodo-1H-pyrazole (33.0 g, 170 mmol, CAS #3469-69-0) in THF (180 mL) was added DIAD (51.6 g, 255 mmol, 49.6 mL) and PPh; (66.9 g, 255 mmol) at 0° C. The mixture was stirred at 25° C., for 12 h. On completion, the reaction mixture was diluted with water (100 mL) and extracted with EtOAc (150 mL×3). The combined organic layers were washed with brine (300 mL×2), dried over Na₂SO₄ and evaporated. The residue was purified by column chromatography (SiO₂, Petroleum ether:Ethyl acetate=20:1 to Petroleum ether:Ethyl acetate=5:1) to give the title compound (5 g, 11% yield) as a white oil. LC-MS (ESI⁺) m/z 265.0 (M+H)⁺.

Step 2—(E)-4-iodo-5-methyl-1-neopentyl-1H-pyrazole. To a solution of 1-(2,2-dimethylpropyl)-4-iodo-pyrazole (4.9 g, 18.5 mmol) in THF (50 mL) was added LDA (2 M, 23.1 mL) at −78° C., and the mixture was stirred for 0.5 h. Then Mel (3.95 g, 27.8 mmol, 1.73 mL) was added and the mixture was stirred at −78° C., for 12 h. On completion, the reaction was quenched with sat. NH₄Cl (10 mL) in an ice bath, and the mixture was extracted with ethyl acetate (10 mL×3). The combined organic layer was washed with saturated brine (20 mL×3), dried over anhydrous sodium sulfate, filtered and concentrated. The residue was purified by column chromatography (SiO₂, Petroleum ether:Ethyl acetate=50:1 to Petroleum ether:Ethyl acetate=5:1) to give the title compound (1.3 g, 25% yield) as a white oil. LC-MS (ESI⁺) m/z 279.1 (M+H)⁺: ¹H NMR (400 MHZ, DMSO-d₆) δ=7.44 (s, 1H), 3.90-3.88 (m, 2H), 2.26 (s, 3H), 0.91-0.89 (m, 9H).

Step 3—5-Methyl-1-neopentyl-4-(4,4,5,5-tetramethyl-1,3,2-dioxaborolan-2-yl)-1H-pyrazole. To a solution of 1-(2,2-dimethylpropyl)-4-iodo-5-methyl-pyrazole (866 mg, 3.11 mmol), 4,4,5,5-tetramethyl-1,3,2-dioxaborolane (1.20 g, 9.34 mmol, 1.36 mL, CAS #25015-63-8) in ACN (8 mL) was added Pd (dppf) Cl2·CH₂Cl₂ (178 mg, 217 umol) and TEA (945 mg, 9.34 mmol, 1.30 mL). The mixture was stirred at 80° C., for 2 h. On completion, the reaction mixture was diluted with water (10 mL) and extracted with EtOAc (10 mL×3). The combined organic layers were washed with brine (20 mL×2), dried over Na₂SO₄ and evaporated. The filtrate was purified by perp-HPLC (column: Phenomenex luna C18 150*25 mm*10 um; mobile phase: [water (FA)-ACN]: B %: 56%-86%, 10 min) to give the title compound (300 mg, 3% yield) as a white solid. LC-MS (ESI⁺) m/z 279.0 (M+H)⁺: ¹H NMR (400 MHZ, DMSO-d₆) δ=7.47 (s, 1H), 3.83-3.80 (m, 2H), 2.37 (s, 3H), 1.24 (s, 12H), 0.91-0.90 (m, 9H).

### 6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinic acid (Intermediate EZ)

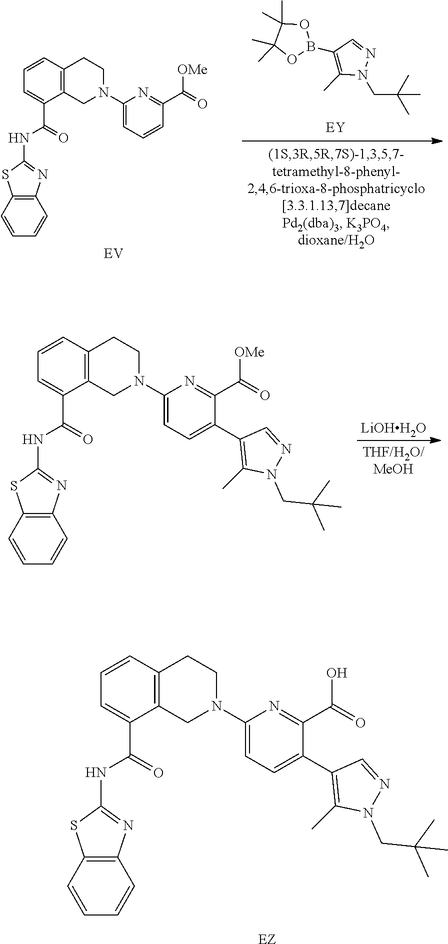

Step 1—Ethyl methyl 6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinate. To a solution of 1-(2,2-dimethylpropyl)-5-methyl-4-(4,4,5,5-tetramethyl-1,3,2-dioxaborolan-2-yl) pyrazole (230 mg, 826 umol, Intermediate EY), methyl 6-[8-(1,3-benzothiazol-2-ylcarbamoyl)-3,4-dihydro-1H-isoquinolin-2-yl]-3-bromo-pyridine-2-carboxylate (360 mg, 689 umol, Intermediate EV) in H₂O (2 mL) and dioxane (2 mL) was added Pd₂(dba); (39.6 mg, 68.9 umol), K₃PO₄ (438 mg, 2.07 mmol), and (1S,3R,5R,7S)-1,3,5,7-tetramethyl-8-phenyl-2,4,6-trioxa-8-phosphatricyclo[3,3,1,1^{3,7}]decane (80.56 mg, 275 umol, L1). The mixture was then stirred at 100° C., for 2 h. On completion, the reaction mixture was diluted with water (5 mL) and extracted with EtOAc (5 mL×3). The combined organic layers were washed with brine (10 mL×2), dried over Na₂SO₄ and evaporated. The residue was purified by column chromatography (SiO₂, Petroleum ether:Ethyl acetate=10:1 to Petroleum ether:Ethyl acetate=1:1) to give the title compound (240 mg, 59% yield) as a yellow solid. LC-MS (ESI⁺) m/z 595.3 (M+H)⁺.

Step 2—6-(8-(Benzo[d]thiazol-2-ylcarbamoyl)-3,4-dihydroisoquinolin-2 (1H)-yl)-3-(5-methyl-1-neopentyl-1H-pyrazol-4-yl)picolinic acid. To a solution of methyl 6-[8-(1,3-benzothiazol-2-ylcarbamoyl)-3,4-dihydro-1H-isoquinolin-2-yl]-3-[1-(2,2-dimethylpropyl)-5-methyl-pyrazol-4-yl]pyridine-2-carboxylate (240 mg, 403 umol) in THF (1 mL), H₂O (1 mL), and MeOH (1 mL) was added LiOH·H₂O (84.6 mg, 2.02 mmol). The mixture was stirred at 25° C., for 12 h. On completion, the mixture was adjusted pH to 4˜5 and concentrated in vacuo. The mixture was diluted with H₂O (5 mL), extracted with EtOAc mL (5 mL×3). The combined organic layer was then washed with brine (5 mL), dried over anhydrous Na₂SO₄, filtered and concentrated under reduced pressure to give the title compound (220 mg) as a yellow solid. LC-MS (ESI⁺) m/z 518.4 (M+H)⁺.

### Tert-butyl 1-(4-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)phenyl)piperidine-4-carboxylic acid (Intermediate FA)

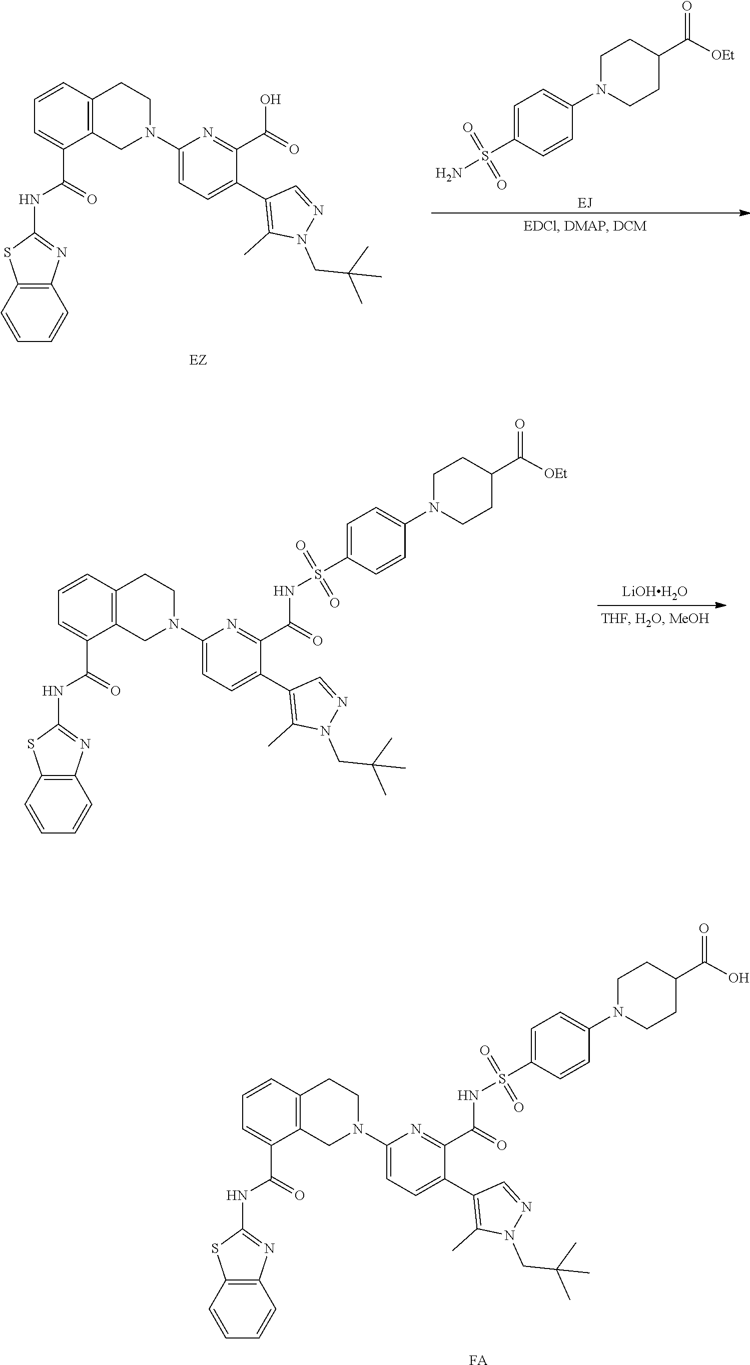

Step 1—Ethyl 1-(4-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)phenyl)piperidine-4-carboxylate. To a solution of 6-[8-(1,3-benzothiazol-2-ylcarbamoyl)-3,4-dihydro-1H-isoquinolin-2-yl]-3-[1-(2,2-dimethylpropyl)-5-methyl-pyrazol-4-yl]pyridine-2-carboxylic acid (210 mg, 361 umol, Intermediate EZ), ethyl 1-(4-sulfamoylphenyl)piperidine-4-carboxylate (146 mg, 470 umol, Intermediate EJ) in DCM (2 mL) was added EDCI (138 mg, 723 umol) and DMAP (110 mg, 904 umol). The mixture was stirred at 25° C., for 12 h. On completion, the reaction mixture was diluted with water (5 mL) and extracted with DCM (5 mL×3). The combined organic layers were washed with brine (10 mL×2), dried over Na₂SO₄ and evaporated. The residue was purified by column chromatography (SiO₂, Petroleum ether:Ethyl acetate=5:1 to Petroleum ether:Ethyl acetate=0:1) to give the title compound (160 mg, 51% yield) as a yellow solid. LC-MS (ESI⁺) m z 875.5 (M+H)⁺.

Step 2—Tert-butyl 1-(4-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)phenyl)piperidine-4-carboxylic acid. To a solution of ethyl 1-[4-[[6-[8-(1,3-benzothiazol-2-ylcarbamoyl)-3,4-dihydro-1H-isoquinolin-2-yl]-3-[1-(2,2-dimethylpropyl)-5-methyl-pyrazol-4-yl]pyridine-2-carbonyl]sulfamoyl]phenyl]piperidine-4-carboxylate (140 mg, 160 umol) in MeOH (0.5 mL), H₂O (0.5 mL), and THF (0.5 mL) was added LiOH·H₂O (33.5 mg, 799 umol). The mixture was stirred at 25° C., for 1 h. On completion, the mixture was adjusted pH to 4˜5 and concentrated in vacuo. The mixture was diluted with H₂O (3 mL), and extracted with EtOAc mL (3 mL×3). The combined organic layer was then washed with brine (5 mL), dried over anhydrous Na₂SO₄, filtered and concentrated under reduced pressure to the title compound (120 mg) as a white solid. LC-MS (ESI⁺) m/z 874.4 (M+H)⁺.

### 1-(2-Ethylbutyl)-5-methyl-4-(4,4,5,5-tetramethyl-1,3,2-dioxaborolan-2-yl)-1H-pyrazole (Intermediate FB)

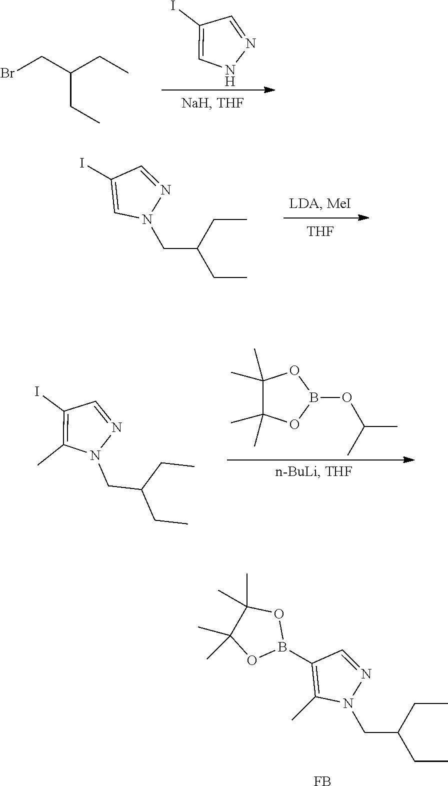

Step 1—1-(2-Ethylbutyl)-4-iodo-1H-pyrazole. A mixture of 4-iodo-1H-pyrazole (28.2 g, 145 mmol, CAS #3469-69-0) in DMF (200 mL) and NaH (7.27 g, 182 mmol, 60% dispersion in mineral oil) was cooled to 0° C., and stirred 1 h. Then 3-(bromomethyl) pentane (20 g, 121 mmol, CAS #3814-34-4) was added and the mixture was stirred at 65° C., for 24 h. On completion, the reaction mixture was partitioned between water (200 mL) and ethyl acetate (100 mL). The aqueous layer was extracted with additional ethyl acetate (250 mL×d). The combined organic layers were washed with brine, dried over Na₂SO₄, filtered, and concentrated. The residue was purified by column chromatography (SiO₂, Petroleum ether/Ethyl: acetate=20/1-10/1) to give the title compound (13 g, 39% yield) as a white solid. ¹H NMR (400 MHZ, CDCl₃) δ=7.50 (s, 1H), 7.40 (s, 1H), 4.02 (d, J=7.2 Hz, 2H), 1.82 (spt, J=6.4 Hz, 1H), 1.33-1.23 (m, 4H), 0.90 (t, J=7.6 Hz, 6H).

Step 2—1-(2-Ethylbutyl)-4-iodo-5-methyl-1H-pyrazole. To a solution of 1-(2-ethylbutyl)-4-iodo-1H-pyrazole (5 g, 18.0 mmol) in THF (50 mL) was added LDA (2 M, 14.4 mL) at −70° C., and the mixture was stirred at −70° C., for 0.5 h. Iodomethane (3.83 g, 27.0 mmol) was added in dropwise and the mixture was stirred at −70 to 25° C., over 12 h. On completion, the reaction mixture was quenched with NH₄Cl solution (200 mL), and then extracted with EtOAc (200 mL×3). The combined organic layers were washed with brine (200 mL×3), dried over Na₂SO₄, filtered and concentrated under reduced pressure to give a residue. The residue was purified by column chromatography (SiO₂, Petroleum ether/Ethyl acetate=20/1-10/1) to give the title compound (4.72 g, 90% yield) as a yellow solid. ¹H NMR (400 MHZ, CDCl₃) δ=7.44 (s, 1H), 3.97 (d, J=7.6 Hz, 2H), 2.28 (s, 3H), 1.87-1.78 (m, 1H), 1.34-1.26 (m, 4H), 0.89 (t, J=7.6 Hz, 6H).

Step 3—1-(2-Ethylbutyl)-5-methyl-4-(4,4,5,5-tetramethyl-1,3,2-dioxaborolan-2-yl)-1H-pyrazole. To solution of 1-(2-ethylbutyl)-4-iodo-5-methyl-1H-pyrazole (4.22 g, 14.4 mmol) in THF (42 mL) was added n-BuLi (2.5 M, 8.67 mL) at −78° C., under N₂ and the mixture was stirred at −78° C., for 1 h, Then 2-isopropoxy-4,4,5,5-tetramethyl-1,3,2-dioxaborolane (3.49 g, 18.8 mmol, CAS #61676-62-8) was added and the mixture was stirred at −78° C., for 1 h. On completion, the reaction mixture was quenched with NH₄Cl (20 mL), and then extracted with EtOAc (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=20/1-10/1) to give the title compound (2 g, 47% yield) as a colorless oil. ¹H NMR (400 MHZ, CDCl₃) δ=7.70 (s, 1H), 3.91 (d, J=7.6 Hz, 2H), 2.43 (s, 3H), 1.96-1.83 (m, 1H), 1.31 (s, 14H), 0.89 (t, J=7.6 Hz, 6H).

### 6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinic acid (Intermediate FC)

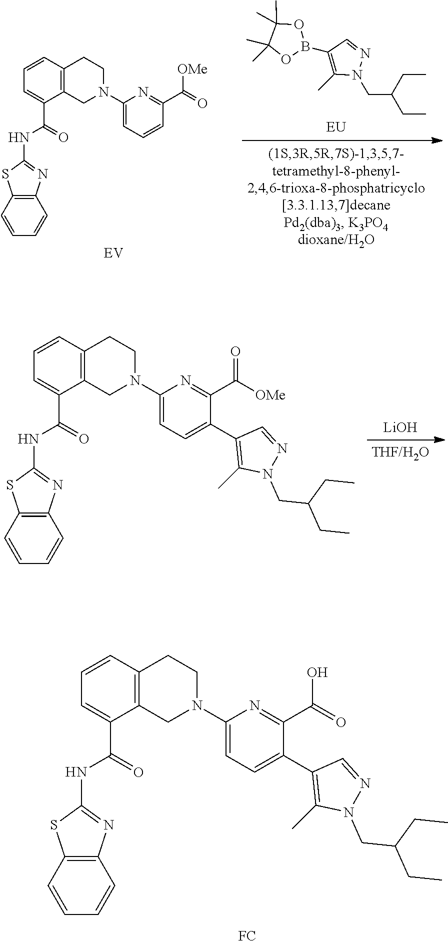

Step 1—Methyl 6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinate. To a solution of 1-(2-ethylbutyl)-5-methyl-4-(4,4,5,5-tetramethyl-1,3,2-dioxaborolan-2-yl)-1H-pyrazole (949 mg, 3.25 mmol, Intermediate FB) and methyl 6-(8-(benzo[d]thiazol-2-ylcarbamoyl)-3,4-dihydroisoquinolin-2 (1H)-yl)-3-bromopicolinate (1 g, 1.91 mmol, Intermediate EV) in dioxane (7 mL) and H₂O (7 mL) was added Pd₂(dba); (175 mg, 191 umol), (1S,3R,5R,7S)-1,3,5,7-tetramethyl-8-phenyl-2,4,6-trioxa-8-phosphatricyclo[3,3,1,1^{3,7}]decane (223 mg, 764 umol, L1) and K₃PO₄ (1.22 g, 5.73 mmol). The mixture was then stirred at 80° C., for 2 h under N₂. On completion, the reaction mixture was poured into water (2 mL), and then extracted with EtOAc (2 mL×3). The combined organic layers were washed with brine (2 mL×3), dried over Na₂SO₄, filtered and concentrated under reduced pressure to give a residue. The residue was purified by column chromatography (SiO₂, Dichloromethane:Methanol=20/1-10/1) to give the title compound (490 mg, 42% yield) as a yellow solid. ¹H NMR (400 MHZ, DMSO-d₆) δ=12.86 (s, 1H), 8.06-7.98 (m, 2H), 7.79 (br d, J=8.0 Hz, 1H), 7.63-7.57 (m, 2H), 7.52 (d, J=8.8 Hz, 1H), 7.44 (br d, J=7.6 Hz, 1H), 7.39-7.37 (m, 1H), 7.21 (s, 1H), 6.98 (d, J=9.2 Hz, 1H), 4.97-4.91 (m, 2H), 3.94-3.82 (m, 4H), 3.51 (s, 3H), 3.04-2.98 (m, 2H), 2.08 (s, 2H), 1.81-1.71 (m, 1H), 1.23 (br d, J=12.8 Hz, 4H), 0.83 (t, J=7.6 Hz, 6H).

Step 2—6-(8-(Benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinic acid. A solution of methyl 6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinate in MeOH (1 mL), H₂O (1 mL) and THF (3 mL) was added LiOH (77.1 mg, 3.22 mmol). Then the mixture was stirred at 20-40° C., for 5 h. On completion, the mixture was concentrated and filtered, then the mixture was diluted with DCM (1 ml) and water (1 ml). The aqueous phase was adjusted pH-10 to 6 with HCl (1 M), then the mixture was extracted with DCM: THF, 4:1 (2 ml×3). The organic phase was dried over Na₂SO₄ and concentrated. The residue was purified by column chromatography (SiO₂, Dichloromethane: Methanol=40/1-10/1) to give the title compound (260 mg, 54% yield) as a yellow solid. ¹H NMR (400 MHz, DMSO-d₆) δ=13.21-12.51 (m, 2H), 8.04 (d, J=7.6 Hz, 1H), 7.79 (d, J=8.0 Hz, 1H), 7.62 (d, J=7.6 Hz, 1H), 7.48 (d, J=8.8 Hz, 2H), 7.46-7.42 (m, 1H), 7.39-7.32 (m, 2H), 7.28 (s, 1H), 6.93 (d, J=8.8 Hz, 1H), 4.95 (s, 2H), 3.93-3.85 (m, 4H), 3.00 (br t, J=5.6 Hz, 2H), 2.12 (s, 3H), 1.75 (td, J=6.4, 12.8 Hz, 1H), 1.28-1.22 (m, 4H), 0.83 (t, J=7.6 Hz, 6H).

### 1-(4-(N-(6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinoyl)sulfamoyl)phenyl)piperidine-4-carboxylic acid (Intermediate FD)

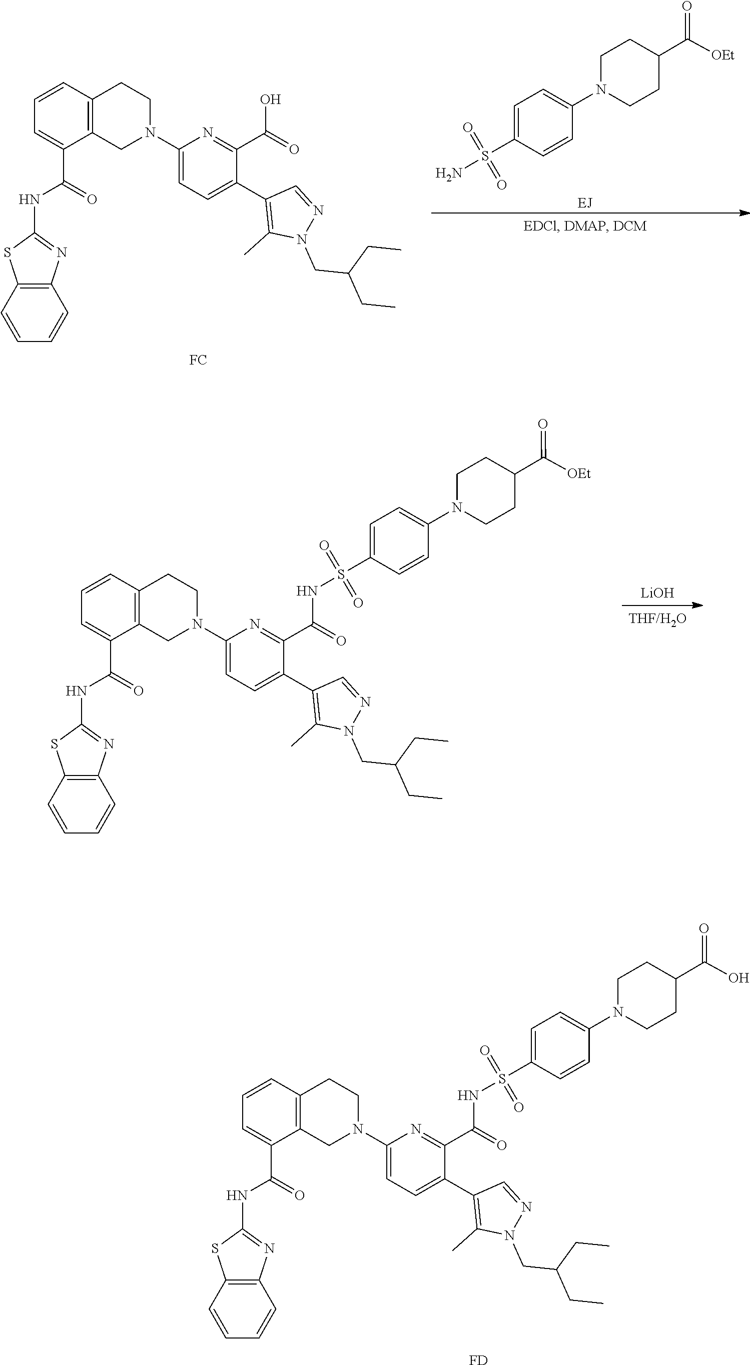

Step 1—Ethyl 1-(4-(N-(6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinoyl)sulfamoyl)phenyl)piperidine-4-carboxylate. To a solution of 6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinic acid (240 mg, 404 umol, Intermediate FC) and ethyl 1-(4-sulfamoylphenyl)piperidine-4-carboxylate (189 mg, 605 umol, Intermediate EJ) in DCM (2 mL) was added DMAP (173 mg, 1.41 mmol). Then EDCI (232 mg, 1.21 mmol) was added in batches and the mixture was stirred at 25° C., for 2 h. On completion, the reaction mixture was poured into water (2 mL), and then extracted with DCM (2 mL×3). The combined organic layers were washed with brine (2 mL×3), dried over Na₂SO₄, filtered and concentrated under reduced pressure to give a residue. The residue was purified by column chromatography (SiO₂, Dichloromethane:Methanol=20/1-10/1) to give the title compound (127 mg, 35% yield) as a white solid. ¹H NMR (400 MHZ, DMSO-d₆) δ=8.13 (d, J=6.4 Hz, 1H), 8.03 (d, J=8.4 Hz, 1H), 7.79 (d, J=8.0 Hz, 1H), 7.63 (d, J=7.6 Hz, 1H), 7.58 (d, J=9.2 Hz, 2H), 7.46 (s, 1H), 7.39-7.32 (m, 2H), 7.15 (s, 1H), 6.94-6.85 (m, 3H), 6.71 (d, J=7.2 Hz, 1H), 4.93 (s, 2H), 4.08 (q, J=7.2 Hz, 2H), 3.92 (br t, J=6.0 Hz, 2H), 3.81-3.74 (m, 4H), 3.01 (br s, 2H), 2.95-2.86 (m, 2H), 1.92-1.84 (m, 5H), 1.72-1.54 (m, 3H), 1.26-1.21 (m, 4H), 1.21-1.16 (m, 4H), 0.83 (t, J=7.6 Hz, 6H).

Step 2—1-(4-(N-(6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinoyl)sulfamoyl)phenyl)piperidine-4-carboxylic acid. A solution of ethyl 1-(4-(N-(6-(8-(benzo[d]thiazol-2-ylcarbamoyl)-3,4-dihydroisoquinolin-2 (1H)-yl)-3-(1-(2-ethylbutyl)-5-methyl-1H-pyrazol-4-yl)picolinoyl)sulfamoyl)phenyl)piperidine-4-carboxylate (127 mg, 143 umol) in THF (1.5 mL) and H₂O (1.5 mL) was added LiOH (13.7 mg, 571 umol), then the mixture was stirred at 20-40° C., for 5 h. On completion, the mixture was concentrated and filtered, then diluted with DCM (1 ml) and water (1 ml). Then the aqueous phase was adjusted to pH=10 to 6 with HCl (1 M), and the mixture was extracted with DCM: THF, 4:1 (2 ml×3). The organic phase was dried over Na₂SO₄ and concentrated. The residue was purified by column chromatography (SiO₂, Dichloromethane:Methanol=40/1-10/1) to give the title compound (100 mg, 81% yield) as a yellow solid. ¹H NMR (400 MHZ, DMSO-d₆) δ=12.93-12.78 (m, 1H), 11.83-11.78 (m, 1H), 8.03 (d, J=7.6 Hz, 1H), 7.82-7.77 (m, 1H), 7.69-7.57 (m, 3H), 7.52-7.41 (m, 3H), 7.40-7.31 (m, 2H), 7.12 (s, 1H), 7.02-6.84 (m, 3H), 4.96 (s, 2H), 3.92 (br t, J=5.6 Hz, 2H), 3.83-3.75 (m, 4H), 3.05-2.99 (m, 2H), 2.98-2.88 (m, 2H), 1.92-1.85 (m, 2H), 1.80 (s, 2H), 1.76 (td, J=3.2, 6.4 Hz, 3H), 1.69-1.64 (m, 1H), 1.22 (quin, J=7.2 Hz, 5H), 0.83 (t, J=7.6 Hz, 6H).

### Methyl 6-sulfamoylhexanoate (Intermediate FE)

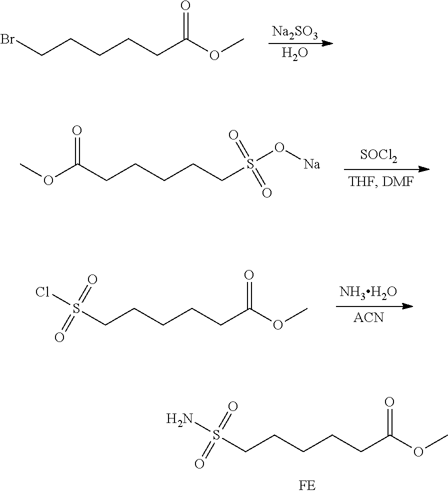

Step 1—Sodium 6-methoxy-6-oxohexane-1-sulfonate. To a solution of methyl 6-bromohexanoate (5.00 g, 23.9 mmol) and Na₂SO; (3.92 g, 31.1 mmol) in H₂O (50 mL). The mixture was stirred at 120° C., for 12 h. On completion, the reaction mixture was lyophilized to give the title compound (8 g) as a white solid.

Step 2—Methyl 6-(chlorosulfonyl) hexanoate. To a solution of sodium 6-methoxy-6-oxohexane-1-sulfonate (4.30 g, 18.5 mmol) in THF (40 mL) and DMF (2 mL) was added thionyl chloride (19.0 g, 160 mmol, 11.6 mL) dropwise at 0° C., under N₂. Then the mixture was heated to 70° C., and stirred for 1 h. On completion, the mixture was concentrated in vacuo to give title compound (4.23 g) as a yellow liquid.

Step 3—Methyl 6-sulfamoylhexanoate. Methyl 6-chlorosulfonylhexanoate (5.00 g, 21.9 mmol) was diluted with ACN (50 mL), and the resulting suspension was added into NH₃·H₂O (32.7 g, 233 mmol, 35.9 mL) at 0° C., under N₂. Then the mixture was stirred for 30 min. On completion, the reaction mixture was diluted with EtOAc (50 mL), the organic layer was separated and washed with H₂O (20 mL×1), brine (20 mL×1), dried over sodium sulfate and evaporated. The residue was purified by column chromatography (SiO₂, PE/EA=3/1) to give the title compound (1.70 g, 37% yield) as a white solid. ¹H NMR (400 MHZ, CDCl₃) δ=4.92 (s, 2H), 3.68 (s, 2H), 3.18-3.08 (m, 2H), 2.34 (t, J=7.2 Hz, 2H), 1.88 (td, J=8.0, 15.6 Hz, 2H), 1.72-1.64 (m, 2H), 1.54-1.44 (m, 2H).

### 3-(1-(cyclohexylmethyl)-5-methyl-1H-pyrazol-4-yl)-6-(8-(thiazolo[4,5-b]pyridin-2-ylcarbamoyl)-3,4-dihydroisoquinolin-2 (1H)-yl)picolinic acid (Intermediate FF)

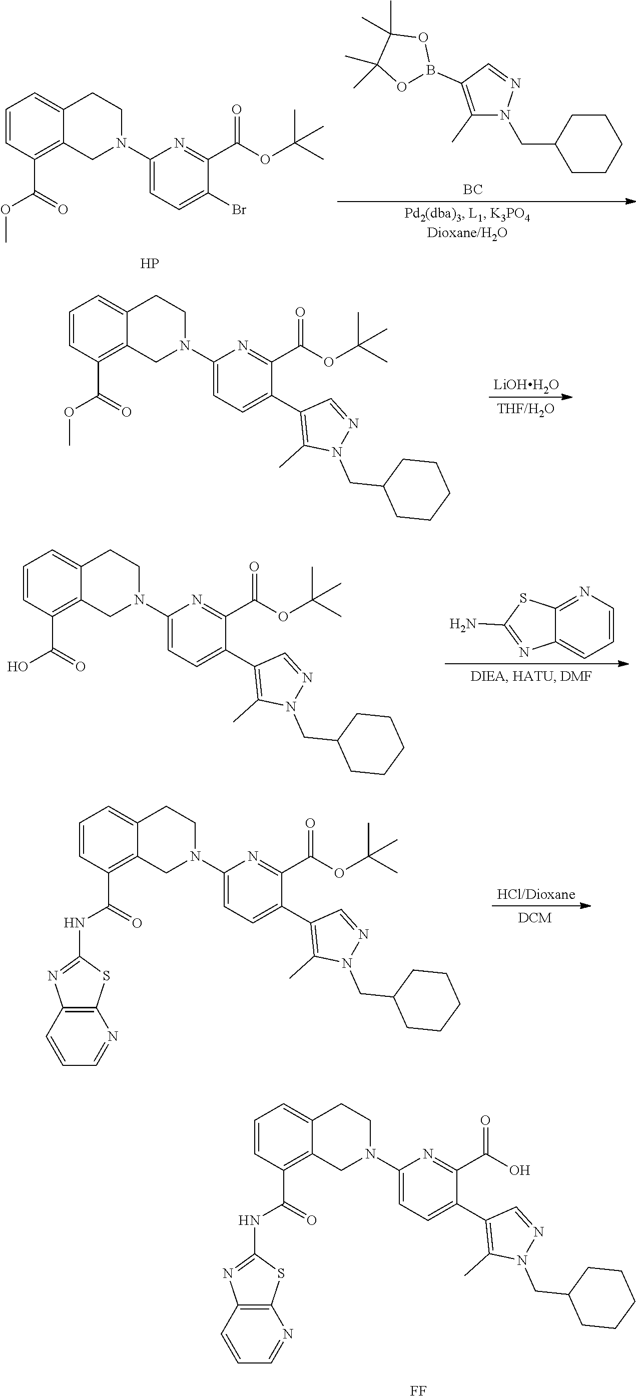

Step 1—Methyl-2-(6-(tert-butoxycarbonyl)-5-(1-(cyclohexylmethyl)-5-methyl-1H-pyrazol-4-yl) pyridin-2-yl)-1,2,3,4-tetrahydroisoquinoline-8-carboxylate. A mixture of 1-(cyclohexylmethyl)-5-methyl-4-(4,4,5,5-tetramethyl-1,3,2-dioxaborolan-2-yl)-1H-pyrazole (12.2 g, 40.2 mmol. Intermediate HP), methyl 2-(5-bromo-6-(tert-butoxycarbonyl) pyridin-2-yl)-1,2,3,4-tetrahydroisoquinoline-8-carboxylate (12 g, 26.8 mmol. Intermediate BC). Pd₂(dba); (1.97 g, 2.15 mmol). (1S,3R,5R,7S)-1,3,5,7-tetramethyl-8-phenyl-2,4,6-trioxa-8-phosphatricyclo[3,3,1,1^{3,7}]decane (2.74 g, 9.39 mmol, L1) and K₃PO₄ (17.0 g, 80.4 mmol) in dioxane (150 mL) and H₂O (50 mL) was degassed and purged with N₂ for 3 times. Then the mixture was stirred at 100° C., for 2 hrs under N₂ atmosphere. The reaction mixture was diluted with H₂O (50 mL) and extracted with EtOAc (80 mL×3). The combined organic layers were dried over Na₂SO₄ filtered and concentrated under reduced pressure to give a residue. The residue was purified by column chromatography (SiO₂. Petroleum ether/Ethyl acetate=100/1 to 10/1) to give the title compound (12 g, 81% yield) as a yellow solid. LC-MS (ESI) m/z 545.6 (M+H).

Step 2—2-(6-(Tert-butoxycarbonyl)-5-(1-(cyclohexylmethyl)-5-methyl-1H-pyrazol-4-yl) pyridin-2-yl)-1,2,3,4-tetrahydroisoquinoline-8-carboxylic acid. To a solution of methyl 2-(6-(tert-butoxycarbonyl)-5-(1-(cyclohexylmethyl)-5-methyl-1H-pyrazol-4-yl) pyridin-2-yl)-1,2,3,4-tetrahydroisoquinoline-8-carboxylate (12 g, 22.0 mmol) in THF (50 mL) and H₂O (50 mL) was added LiOH·H₂O (4.62 g, 110 mmol). The mixture was stirred at 25° C., for 12 hrs. On completion, the pH of reaction mixture was adjust to acid condition (pH=3-4) by HCl (1 M) and then extracted with EtOAc (80 mL×3). The combined organic layers were dried over Na₂SO₄ filtered and concentrated under reduced pressure to give the title compound (12 g) as a yellow solid. LC-MS (ESI) m/z 531.1 (M+H).

Step 3—Tert-butyl 3-(1-(cyclohexylmethyl)-5-methyl-1H-pyrazol-4-yl)-6-(8-(thiazolo]5,4-b]pyridin-2-ylcarbamoyl)-3,4-dihydroisoquinolin-2 (1H)-yl)picolinate. To a solution of 2-[6-tert-butoxycarbonyl-5-[1-(cyclohexylmethyl)-5-methyl-pyrazol-4-yl]-2-pyridyl]-3,4-dihydro-1H-isoquinoline-8-carboxylic acid (5.85 g, 11.0 mmol) and thiazolo[5,4-b]pyridin-2-amine (2 g, 13.23 mmol. CAS #31784-70-0) in DMF (60 mL) was added DIEA (4.27 g, 33.07 mmol) and HATU (4.19 g, 11.0 mmol), then the mixture was stirred at 50° C., for 16 h. On completion, the reaction mixture was diluted with H₂O (30 mL) and extracted with EtOAc (50 mL×3). The combined organic layers were washed with brine (50 mL×3), dried over Na₂SO₄ filtered and concentrated under reduced pressure to give a residue. The residue was purified by column chromatography (SiO₂. Petroleum ether/Ethyl acetate=10/1 to 0/1) to give the title compound (4.5 g, 63% yield) as a yellow solid. LC-MS (ESI) m/z 785.3 (M+H).

Step 4—3-(1-(Cyclohexylmethyl)-5-methyl-1H-pyrazol-4-yl)-6-(8-(thiazolo]5,4-b]pyridin-2-ylcarbamoyl)-3,4-dihydroisoquinolin-2 (1H)-yl)picolinic acid. To a solution of tert-butyl 3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinate (4 g, 6.03 mmol) in DCM (20 mL) was added HCl/dioxane (4 M, 1.51 mL). The mixture was stirred at 40° C., for 12 hrs. On completion, the reaction mixture was concentrated under reduced pressure to give the title compound (3 g) as a white solid. LC-MS (ESI) m/z 608.4 (M+H)⁺; ¹H NMR (400 MHZ, DMSO-d₆) δ=13.63-11.80 (m, 2H), 8.52 (dd, J=1.4, 4.7 Hz, 1H), 8.16 (dd, J=1.3, 8.2 Hz, 1H), 7.63 (d, J=7.3 Hz, 1H), 7.55-7.48 (m, 2H), 7.47-7.42 (m, 1H), 7.41-7.34 (m, 1H), 7.27 (s, 1H), 6.96 (d, J=8.8 Hz, 1H), 4.96 (s, 2H), 4.00-3.73 (m, 4H), 3.02 (br t, J=5.8 Hz, 2H), 2.11 (s, 3H), 1.79 (tdd, J=3.6, 7.5, 14.8 Hz, 1H), 1.70-1.59 (m, 3H), 1.53 (br d, J=11.3 Hz, 2H), 1.23-1.09 (m, 3H), 1.03-0.90 (m, 2H).

### 6-(N-(3-(1-(cyclohexylmethyl)-5-methyl-1H-pyrazol-4-yl)-6-(8-(thiazolo[4,5-b]pyridin-2-ylcarbamoyl)-3,4-dihydroisoquinolin-2 (1H)-yl)picolinoyl)sulfamoyl)hexanoic acid (Intermediate FG)

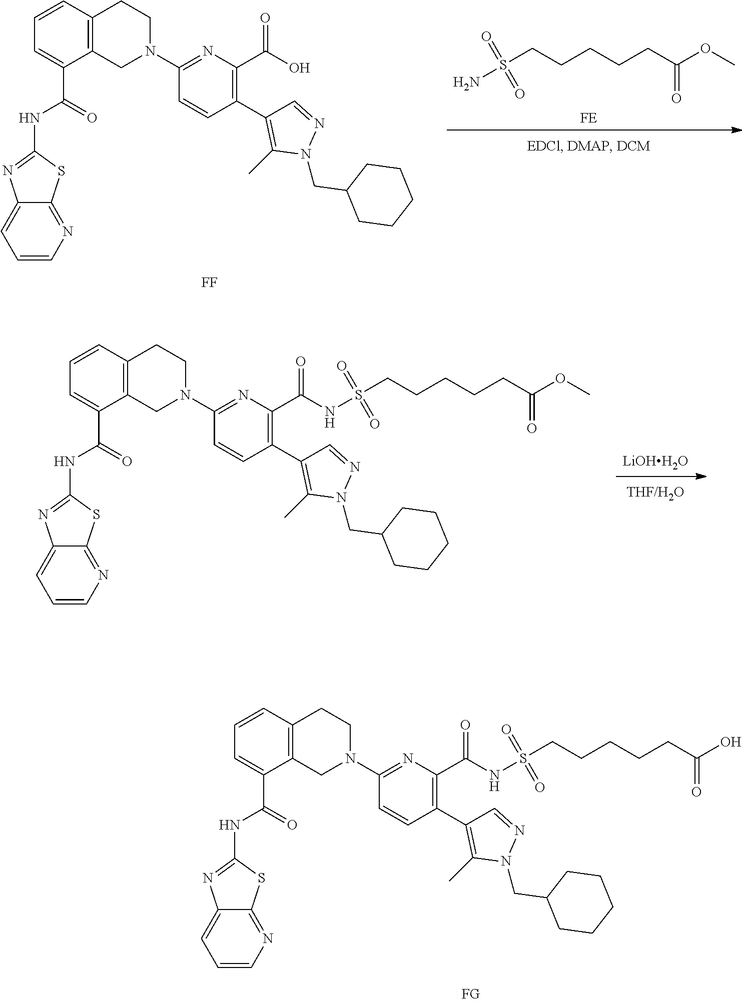

Step 1—Methyl 6-(N-(3-(1-(cyclohexylmethyl)-5-methyl-1H-pyrazol-4-yl)-6-(8-(thiazolo[4,5-b]pyridin-2-ylcarbamoyl)-3,4-dihydroisoquinolin-2 (1H)-yl)picolinoyl)sulfamoyl)hexanoate. To a stirred solution of methyl 6-sulfamoylhexanoate (344.3 mg, 1.7 mmol, Intermediate FE) and 3-(1-(cyclohexylmethyl)-5-methyl-1H-pyrazol-4-yl)-6-(8-(thiazolo[4,5-b]pyridin-2-ylcarbamoyl)-3,4-dihydroisoquinolin-2 (1H)-yl)picolinic acid (500.0 mg, 822.7 umol, Intermediate FF) in DCM (5 mL) at 25° C., was added EDCI (315.4 mg, 1.65 mmol) and DMAP (251.3 mg, 2.10 mmol), then the mixture was stirred at 25° C., for 12 h. On completion, the reaction mixture was quenched with H₂O (10 mL) and extracted with DCM (15 mL×3). The combined organic layers were washed with brine (4 mL×3), dried over Na₂SO₄ and evaporated. The crude product was purified by reversed-phase HPLC (0.1% FA condition, 5%-62%, 40 min) to give the title compound (306.0 mg, 47% yield) as an orange solid. LC-MS (ESI⁺) m/z 799.4 (M+H)⁺.

Step 2—6-(N-(3-(1-(cyclohexylmethyl)-5-methyl-1H-pyrazol-4-yl)-6-(8-(thiazolo[4,5-b]pyridin-2-ylcarbamoyl)-3,4-dihydroisoquinolin-2 (1H)-yl)picolinoyl)sulfamoyl)hexanoic acid. To a solution of methyl 6-(N-(3-(1-(cyclohexylmethyl)-5-methyl-1H-pyrazol-4-yl)-6-(8-(thiazolo[4,5-b]pyridin-2-ylcarbamoyl)-3,4-dihydroisoquinolin-2 (1H)-yl)picolinoyl)sulfamoyl)hexanoate (306.0 mg, 383.0 umol) in THF (3 mL) and H₂O (3 mL) was added LiOH·H₂O (80.4 mg, 1.91 mmol), then the mixture was stirred at 25° C., for 2 h. On completion, the reaction mixture was filtered and extracted with DCM (5 mL×2) and the organic layer was discarded. The pH of the aqueous phase was adjusted to 4˜5 by addition 2M HCl and extracted with DCM: THF, 3:1 (6 mL×8). The combined organic layers were dried over Na₂SO₄ and evaporated to give the title compound (300 mg) as an orange solid. LC-MS (ESI⁺) m/z 785.3 (M+H)⁺.

### ((2S,4R)-1-((S)-2-amino-3-methylbutanoyl)-N-((S)-1-(4-ethynylphenyl)ethyl)-4-hydroxypyrrolidine-2-carboxamide (Intermediate FH)

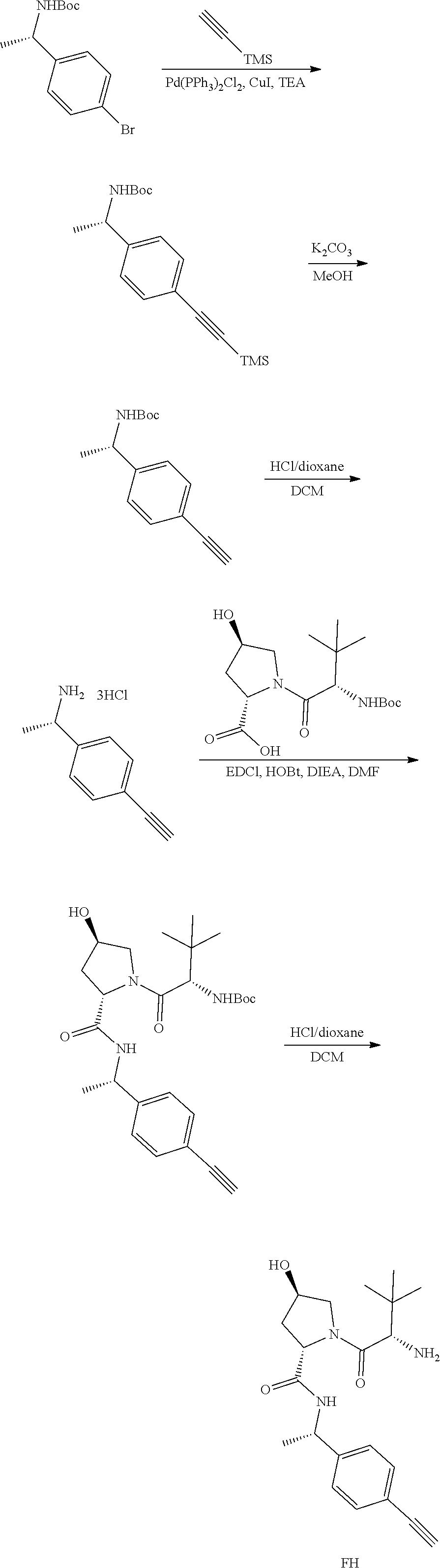

Step 1—(S)-tert-butyl (1-(4-((trimethylsilyl)ethynyl)phenyl)ethyl)carbamate. To a solution of(S)-tert-butyl (1-(4-bromophenyl)ethyl)carbamate (150 g, 499 mmol) and ethynyltrimethylsilane (147 g, 1.50 mol) in TEA (1.50 L) was added CuI (1.95 g, 10.2 mmol) and Pd(PPh₃)₂Cl₂ (17.4 g, 24.9 mmol) under N₂, then the mixture was stirred at 80° C., for 16 rs. On completion, the mixture was cooled to 20° C., filtered and added EtOAc (2.50 L), washed with water (3.00 L×4). The organic layer was washed with brine (1.00 L), dried over Na₂SO₄, filtered and concentrated to give a residue. The residue was purified by silica gel chromatography eluted with Petroleum ether/Ethyl acetate to give the title compound (114 g, 72% yield) as a yellow oil. LC-MS (ESI⁺) m/z 262.2 (M-55)⁺.

Step 2—(S)-tert-butyl (1-(4-ethynylphenyl)ethyl)carbamate. A solution of(S)-tert-butyl (1-(4-((trimethylsilyl)ethynyl)phenyl)ethyl)carbamate (95.0 g, 299 mmol) and K₂CO; (86.1 g, 623 mmol) in MeOH (700 mL) was stirred at 25° C., for 2 hrs. On completion, the mixture was filtered and concentrated to give the title compound (75.0 g) as a gray solid. LC-MS (ESI⁺) m/z 190.1 (M-55)⁺.

Step 3—(S)-1-(4-ethynylphenyl)ethanamine. To a solution of HCl/dioxane (4 M, 300 mL) was added a solution of(S)-tert-butyl (1-(4-ethynylphenyl)ethyl)carbamate (74.0 g, 301 mmol) in DCM (700 mL), then the mixture was stirred at 25° C., for 1 hr. On completion, the mixture was concentrated to give the title compound (80.0 g, 3 HCl) as a gray solid. ¹HNMR (400 MHZ, CDCl₃) δ 8.72 (br s, 3H), 7.54 (q, J=7.82 Hz, 4H), 4.40 (br s, 1H), 4.24 (s, 1H), 1.51 (br d, J=6.85 Hz, 3H).

Step 4—Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl) carbamate. To a solution of(S)-1-(4-ethynylphenyl)ethanamine (47.7 g, 187 mmol, 3 HCl) in DMF (400 mL) was added DIEA (66.7 g, 516 mmol), and the mixture was stirred at 25° C., for 0.25 hr. Then (2S,4R)-1-((S)-2-((tert-butoxycarbonyl)amino)-3,3-dimethylbutanoyl)-4-hydroxypyrrolidine-2-carboxylic acid (43.0 g, 124 mmol), EDCI (28.7 g, 149 mmol) and HOBt (20.2 g, 149 mmol) was added and the mixture was stirred at 25° C., for 3 hrs. On completion, the mixture was poured into water (3.00 L), and extracted with EtOAc (1.00 L×4). The organic layer was washed with water (2.00 L×4), brine (1.50 L), dried over Na₂SO₄, filtered and concentrated. The crude product was purified by silica gel chromatography eluted with Petroleum ether/Ethyl acetate (I/O to 0/1) to give the title compound (45.0 g) as a light yellow solid LC-MS (ESI⁺) m/z 472.1 (M+1)⁺. ¹HNMR (400 MHZ, CDCl₃) δ 7.37 (br d, J=7.70 Hz, 1H), 7.27 (s, 2H), 7.08 (d, J=8.07 Hz, 2H), 5.07 (br d, J=9.05 Hz, 1H), 4.84 (quin, J=7.06 Hz, 1H), 4.52 (t, J=7.89 Hz, 1H), 4.29 (br s, 1H), 4.02 (d, J=9.17 Hz, 1H), 3.84 (br d, J=11.25 Hz, 1H), 3.24-3.35 (m, 2H), 2.86 (s, 1H), 2.19-2.33 (m, 1H), 1.94 (s, 1H), 1.18-1.30 (m, 12H), 0.83 (s, 9H).

Step 5—(2S,4R)-1-((S)-2-amino-3,3-dimethylbutanoyl)-N-((S)-1-(4-ethynylphenyl)ethyl)-4-hydroxypyrrolidine-2-carboxamide. To a solution of tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl) carbamate (40.0 g, 84.8 mmol) in DCM (400 mL) was added HCl/dioxane (4 M, 26.0 mL) at 0-10° C., then the mixture was stirred at 10-20° C., for 16 hrs. On completion, the mixture was added dropwise into n-heptane (2.00 L), then the precipitate was filtered and the filter cake was collected. The residue was dissolved with DCM (400 mL), dissociated with basic resin (200 g), and stirred at 20° C., for 2 hrs. The mixture was then filtered and concentrated to give a residue. The residue was purified by prep-HPLC (NH₃·H₂O condition), and freeze-dried to give the title compound (19.8 g, 97% yield) as a white solid. LC-MS (ESI′) m/z 372.1 (M+1)⁺: ¹HNMR (400 MHZ, CDCl₃) δ 7.73 (br d, J=6.40 Hz, 1H), 7.46 (d, J=8.16 Hz, 2H), 7.27 (s, 2H), 5.04 (br t, J=7.15 Hz, 1H), 4.74 (br t, J=7.72 Hz, 1H), 4.46 (br s, 1H), 3.75 (br d, J=10.92 Hz, 1H), 3.59 (br dd, J=11.04, 3.76 Hz, 1H), 3.42 (br s, 1H), 3.07 (s, 1H), 2.36 (br d, J=5.65 Hz, 1H), 2.04-2.15 (m, 1H), 1.45 (d, J=6.90 Hz, 3H), 1.02 (s, 9H).

### 2-((tert-butoxycarbonyl)amino)-5-((tert-butyldimethylsilyl)oxy)-3,3-dimethylpentanoic acid (Intermediate FI)

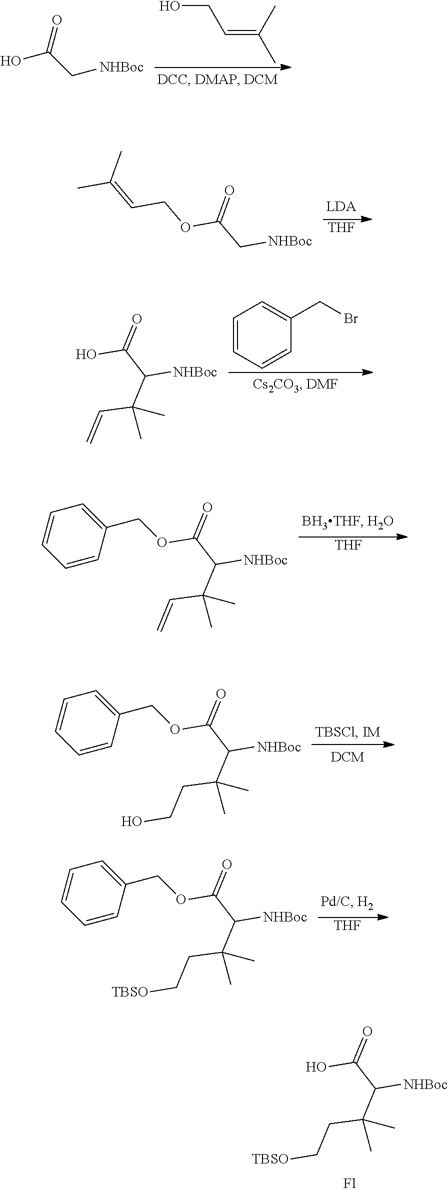

Step 1—3-Methylbut-2-en-1-yl 2-((tert-butoxycarbonyl)amino)acetate. To a solution of 2-(tert-butoxycarbonylamino)acetic acid (20 g, 114 mmol, CAS #4530-20-5) in DCM (300 mL) was added DMAP (2.32 g, 19.0 mmol) and DCC (29.4 g, 143 mmol, 28.9 mL) 3-methylbut-2-en-1-ol (8.19 g, 95.1 mmol, 9.52 mL, CAS #556-82-1) at −5° C. The mixture was stirred at 25° C., for 12 hours. On completion, the reaction mixture was quenched with sat. NH₄Cl (100 mL) at 25° C., and then diluted with EA (100 mL) and extracted with EA (300 mL×3). The combined organic layers were washed with sat. NaCl (300 mL×1), dried over Na₂SO₄, filtered and concentrated under reduced pressure to give a residue. The crude product was purification by MPLC (SiO₂, PE:EA=3/1 to 1:1) to give the title compound (23 g, 99% yield) as a white solid. ¹H NMR (400 MHZ, DMSO-d₆) δ 5.35-5.28 (m, 1H), 5.09 (s, 1H), 4.61 (d, J=7.6 Hz, 2H), 3.87 (d, J=5.6 Hz, 2H), 1.71 (d, J=19.0 Hz, 6H), 1.42 (s, 9H).

Step 2—2-((Tert-butoxycarbonyl)amino)-3,3-dimethylpent-4-enoic acid. A mixture of 3-methylbut-2-enyl 2-(tert-butoxycarbonylamino)acetate (23.5 g, 96.6 mmol), and LDA (2 M, 121 mL) in THF (250 mL) at −65° C., was degassed and purged with N₂ three times. Then the mixture was stirred at 25° C., for 3 hours under N₂ atmosphere. On completion, to the mixture was add toluene (200 mL) at 25° C., and then add 10% H₂SO₄ (100 mL) until the pH=2-3. The organic layer was extracted by 10% NaOH 100 mL (100 mL×1). Then to the water layer was add 20% H₂SO₄ (100 mL) at −10° C., until the pH=2-3, then extracted by EA (300 mL). The organic layer was washed with sat. NaCl (150 mL), dried over Na₂SO₄, filtered and concentrated under reduced pressure to give the title compound (14 g) as a yellows oil. ¹H NMR (400 MHZ, DMSO-d₆) δ 5.85 (dd, J=10.8, 17.2 Hz, 1H), 5.13-5.07 (m, 2H), 5.07-5.00 (m, 1H), 4.16 (d, J=9.2 Hz, 1H), 1.43 (s, 9H), 1.14 (d, J=2.8 Hz, 6H).

Step 3—Benzyl 2-((tert-butoxycarbonyl)amino)-3,3-dimethylpent-4-enoate. To a solution of 2-(tert-butoxycarbonylamino)-3,3-dimethyl-pent-4-enoic acid (7 g, 30 mmol) in DMF (150 mL) was added Cs₂CO; (9.37 g, 28.8 mmol) at 0° C., and the mixture was stirred 30 minutes. Then bromomethylbenzene (4.92 g, 28.8 mmol, 3.42 mL) was added at 0° C. The mixture was then stirred at 25° C., for 4 hrs. On completion, the reaction mixture was quenched with sat. NH₄Cl (50 mL) at 25° C., and then diluted with EA (5 mL) and extracted with EA (100 mL×3). The combined organic layers were washed with sat. NaCl (300 mL), dried over Na₂SO₄, filtered and concentrated under reduced pressure to give a residue. The crude product was purified by MPLC (SiO₂, PE:EA=1:0 to 100:1) to give the title compound (9 g, 94% yield) as a colorless oil. ¹H NMR (400 MHZ, CHLOROFORM-d) & 7.34-7.19 (m, 5H), 5.72 (dd, J=10.8, 17.6 Hz, 1H), 5.13-5.01 (m, 2H), 5.00-4.88 (m, 3H), 4.11 (d, J=9.6 Hz, 1H), 1.35 (s, 9H), 0.99 (d, J=7.2 Hz, 6H).

Step 4—Benzyl 2-((tert-butoxycarbonyl)amino)-5-hydroxy-3,3-dimethylpentanoate. To a 0° C., solution of 2,3-dimethylbut-2-ene (1 M, 17.5 mL) was added dropwise over 20 min of BH₃. THF (1 M, 17.5 mL). After the solution was stirred for an additional 60 minutes at 0° C., benzyl 2-(tert-butoxycarbonylamino)-3,3-dimethyl-pent-4-enoate (5 g, 15.0 mmol) in THF (8 mL) was added dropwise and the resulting solution was allowed to warm to ambient temperature over 1 hour. The solution was re-cooled to 0° C., and excess borane was quenched by the cautious addition of 1 mL of 1:1 THF/ethanol then H₂O₂ (17.7 g, 156 mmol, 15.0 mL, 30% solution) was added. After the reaction mixture was stirred at ambient temperature for 14 hrs. The reaction mixture was quenched with sat. NaHSO₃ (20 mL) at 0° C., and then diluted with EA (10 mL) and extracted with EA (30 mL×3). The combined organic layers were washed with sat. NaCl (50 mL), dried over Na₂SO₄, filtered and concentrated under reduced pressure to give a residue. The crude product was purification by reversed-phase flash chromatography (0.1% FA condition) to give the title compound (1.4 g, 26% yield) as white solid. LC-MS (ESI) m/z 252.2 (M-100+H)

Step 5—Benzyl 2-((tert-butoxycarbonyl)amino)-5-((tert-butyldimethylsilyl)oxy)-3,3-dimethylpentanoate. To a solution of benzyl 2-(tert-butoxycarbonylamino)-5-hydroxy-3,3-dimethyl-pentanoate (5.7 g, 16.2 mmol) in DCM (50 mL) was added imidazole (2.21 g, 32.4 mmol) and TBSCl (2.69 g. 17.8 mmol). The mixture was stirred at 25° C., for 12 hrs. The mixture was stirred at 25° C., for 12 hrs. On completion, the reaction mixture was quenched with H₂O (50 mL) at 25° C., and then diluted with DCM (20 mL) and extracted with DCM (50 mL×3). The combined organic layers were washed with sat. NaCl (50 mL) and dried over Na₂SO₄, filtered and concentrated under reduced pressure to give a residue. The crude product was purified by MPLC (SiO₂, PE:EA=5=30:1 to 10:1) to give the title compound (7 g, 81% yield) as a white solid. LC-MS (ESI) m/z 366.3 (M-Boc+H)⁺.

Step 6-2-((Tert-butoxycarbonyl)amino)-5-((tert-butyldimethylsilyl)oxy)-3,3-dimethylpentanoic acid. To a solution of benzyl 2-(tert-butoxycarbonylamino)-5-[tert-butyl (dimethyl)silyl]oxy-3,3-dimethyl-pentanoate (6.5 g, 14 mmol) in MeOH (70 mL) was added Pd/C (650 mg, 10 wt %) and H₂ (15 psi). The mixture was stirred at 25° C., for 12 hrs. The mixture was filtered to remove the Pd/C and concentrated the solution under reduce pressure to give the title compound (5 g) as white solid. LC-MS (ESI⁺) m/z 376.4 (M+H)⁺.

### (4-Ethynylphenyl)methanamine (Intermediate FJ)

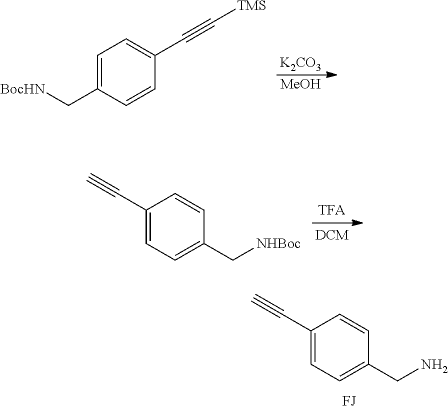

Step 1—Tert-butyl N-[(4-ethynylphenyl)methyl]carbamate. To a solution of tert-butyl N-[[4-(2-trimethylsilylethynyl)phenyl]methyl]carbamate (4.4 g, 14.5 mmol, CAS #680190-95-8) in MeOH (55 mL) was added K₂CO; (4.01 g, 29.0 mmol). The mixture was stirred at 25° C., for 2 hours. The reaction mixture was partitioned between EA (300 mL) and water (100 mL). The organic phase was separated, washed with brine (50 mL×2), dried over Na₂SO₄, filtered and concentrated under reduced pressure to give the title compound (3.3 g) as a yellow solid. LC-MS (ESI⁺) m/z 176.2 (M-55)⁺.

Step 2—(4-Ethynylphenyl)methanamine. To a solution of tert-butyl N-[(4-ethynylphenyl)methyl]carbamate (1.5 g, 6.49 mmol) in DCM (10 mL) was added TFA (2.96 g, 25.9 mmol). The mixture was stirred at 25° C., for 0.5 hour. On completion, the reaction mixture was filtered and concentrated under reduced pressure to give the title compound (837 mg) as obtained as a yellow oil. LC-MS (ESI⁺) m/z 115.2 (M-NH₂)⁺.

### (2S,4R)—N-(4-ethynylbenzyl)-4-hydroxypyrrolidine-2-carboxamide (Intermediate FK)

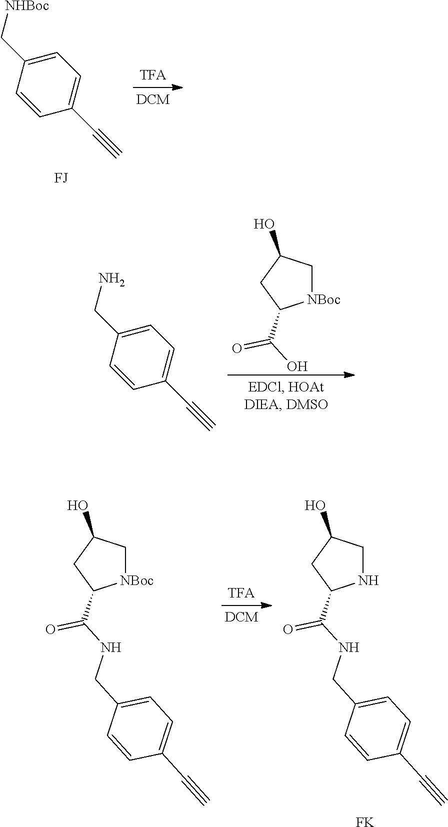

Step 1—(2S,4R)-tert-butyl 2-((4-ethynylbenzyl)carbamoyl)-4-hydroxypyrrolidine-1-carboxylate. To a solution of (4-ethynylphenyl)methanamine (1.5 g, 6.12 mmol, TFA, Intermediate FJ) in DMSO (15 mL) was added EDCI (1.17 g, 6.12 mmol) and HOAt (833 mg, 6.12 mmol, 856 uL) DIEA (3.95 g, 30.6 mmol, 5.33 mL) and (2S,4R)-1-tert-butoxycarbonyl-4-hydroxy-pyrrolidine-2-carboxylic acid (1.41 g, 6.12 mmol, CAS #13726-69-7). The mixture was stirred at 25° C., for 12 hours. The reaction mixture was diluted with EA (20 mL) and extracted with EA (30 mL×3). The combined organic layers were washed with sat. NaCl (50 mL×3), dried over Na₂SO₄, filtered and concentrated under reduced pressure to give a residue. The crude product was purified by MPLC (SiO₂, PE:EA=1:1 to 0:1) to give the title compound (1.8 g, 74% yield) as a white solid. LC-MS (ESI⁺) m/z 245.3 (M-100+H)⁺.

Step 2—(2S,4R)—N-(4-ethynylbenzyl)-4-hydroxypyrrolidine-2-carboxamide. To a solution of tert-butyl (2S,4R)-2-[(4-ethynylphenyl)methylcarbamoyl]-4-hydroxy-pyrrolidine-1-carboxylate (0.5 g, 2 mmol) in DCM (5 mL) was added TFA (1.54 g, 13.5 mmol, 1 mL). The mixture was stirred at 25° C., for 30 mins. The mixture was concentrated under reduce pressure to give the title compound (100 mg, TFA) as a white solid. LC-MS (ESI⁺) m/z 245.3 (M+H)⁺.

Tert-butyl ((S)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate (Intermediate FL) & tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate (Intermediate FM)

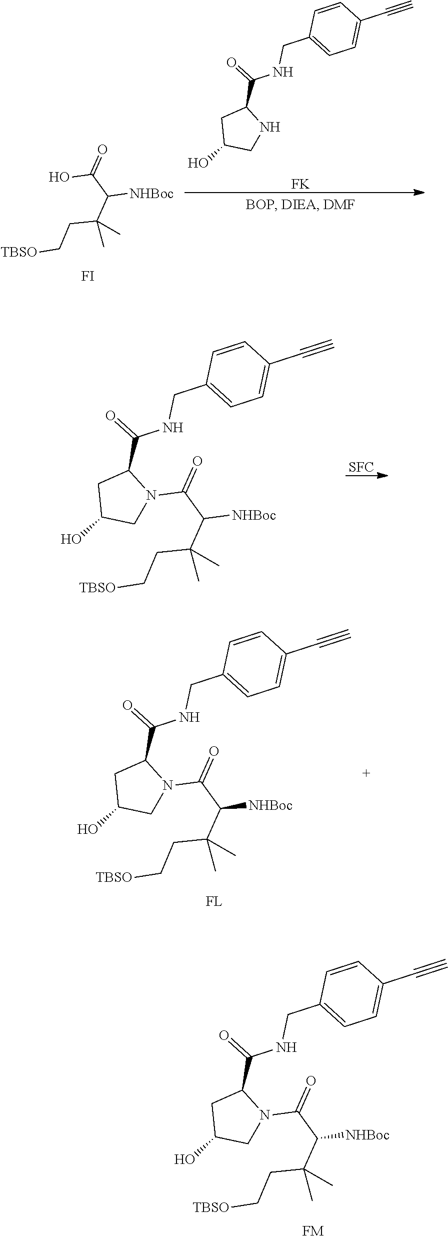

Step 1—(5-((tert-butyldimethylsilyl)oxy)-1-((2S,4R)-2-((4-Tert-butyl ethynylbenzyl)carbamoyl)-4-hydroxypyrrolidin-1-yl)-3,3-dimethyl-1-oxopentan-2-yl) carbamate. To a solution of (2S,4R)-N-[(4-ethynylphenyl)methyl]-4-hydroxy-pyrrolidine-2-carboxamide (715 mg, 2.93 mmol, Intermediate FK) in DMF (15 mL) was added BOP (1.94 g, 4.39 mmol), DIEA (1.89 g, 14.6 mmol, 2.55 mL) and 2-(tert-butoxycarbonylamino)-5-[tert-butyl(dimethyl)silyl]oxy-3,3-dimethyl-pentanoic acid (2.2 g, 5.9 mmol, Intermediate FI). The mixture was stirred at 25° C., for 12 hrs. The mixture was filtered to give the solution. The mixture was purified by reversed-phase flash chromatography (0.1% NH₃·H₂O condition) to give the title compound (300 mg, 15% yield) as white solid. LC-MS (ESI⁺) m/z 602.4 (M+H)+

Step 2—Tert-butyl ((S)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate & tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate. Compound tert-butyl N-[4-[tert-butyl (dimethyl)silyl]oxy-1-[(2S,4R)-2-[(4-ethynylphenyl)methylcarbamoyl]-4-hydroxy-pyrrolidine-1-carbonyl]-2,2-dimethyl-butyl]carbamate (300 mg, 498 umol) was purified by SFC. The crude product was purified by SFC (column: REGIS (S,S) WHELK-01 (250 mm×25 mm, 10 um); mobile phase: [0.1% NH₃H₂O ETOH]: B %: 30%-30%, 2:20 min) to give tert-butyl ((S)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate (140 mg, 43% yield) as white solid (LC-MS (ESI⁺) m/z 602.2 (M+H)⁺) and tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate (170 mg, 53% yield) as white solid (LC-MS (ESI⁺) m/z 602.2 (M+H)⁺).

### (2S,4R)-1-((S)-2-amino-5-hydroxy-3,3-dimethylpentanoyl)-N-(4-ethynylbenzyl)-4-hydroxypyrrolidine-2-carboxamide (Intermediate FN)

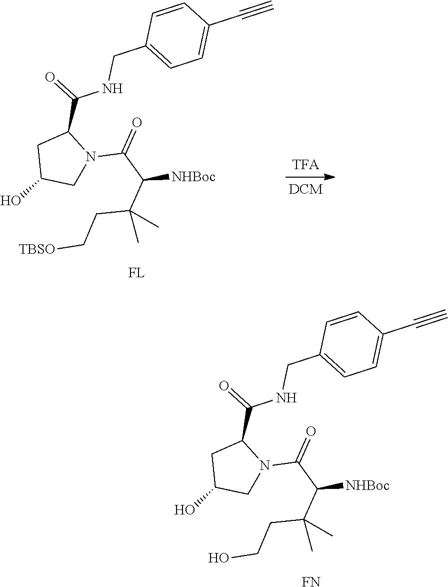

To a solution of tert-butyl N-[(1S)-4-[tert-butyl(dimethyl)silyl]oxy-1-[(2S,4R)-2-[(4-ethynylphenyl)methylcarbamoyl]-4-hydroxy-pyrrolidine-1-carbonyl]-2,2-dimethyl-butyl]carbamate (100 mg, 200 umol, Intermediate FL) in DCM (2 mL) was added TFA (308 mg, 2.70 mmol, 200 uL) and 4 Å molecular sieves (50 mg, 200 umol). The mixture was stirred at 25° C., for 12 hrs. On completion, the mixture was filtered to remove the 4A molecular sieves and concentrated under reduce pressure to give a residue. The crude product was purificated by reversed phase flash chromatography (0.1% NH₃·H₂O) to give the title compound (50 mg, 72% yield) as white solid. LC-MS (ESI⁺) m/z 388.3 (M+H)⁺.

### (2S,4R)-1-((R)-2-amino-5-hydroxy-3,3-dimethylpentanoyl)-N-(4-ethynylbenzyl)-4-hydroxypyrrolidine-2-carboxamide (Intermediate FO)

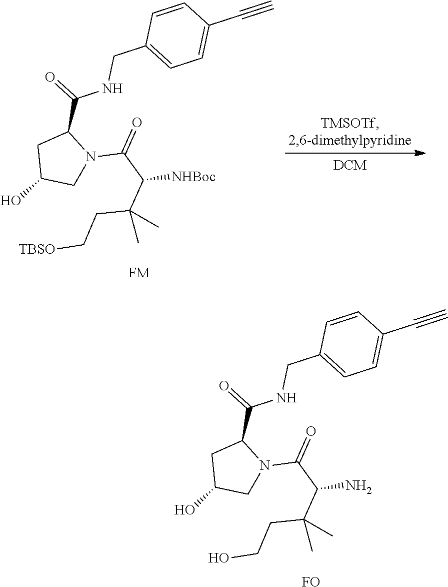

To a solution of tert-butyl ((R)-5-((tert-butyldimethylsilyl)oxy)-1-((2S,4R)-2-((4-ethynylbenzyl)carbamoyl)-4-hydroxypyrrolidin-1-yl)-3,3-dimethyl-1-oxopentan-2-yl) carbamate (100 mg, 166 umol, Intermediate FM) in DCM (0.5 mL) was added TMSOTf (111 mg, 498 umol, 90.0 uL) and 2,6-dimethylpyridine (89.9 mg, 831 umol, 97.7 uL). The mixture was stirred at 25° C., for 2 hrs. The mixture was concentrated under reduce pressure to give a residue. The crude product was purified by reversed phase Flash (0.1% FA) to give the title compound (60 mg, FA Salt) as white solid. LC-MS (ESI⁺) m/z 502.3 (M+114)⁺.

### 3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinic acid (Intermediate FP)

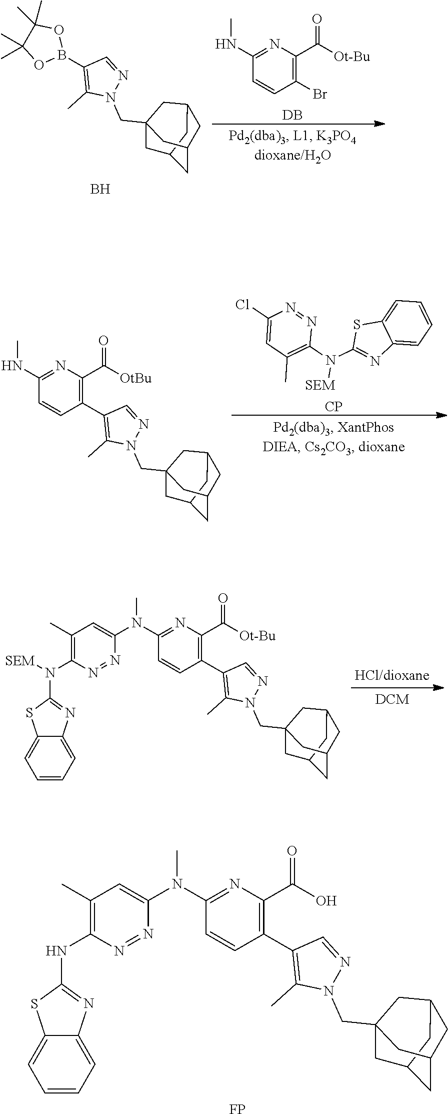

Step 1—Tert-butyl 3-(1-((1s,3s)-adamantan-1-ylmethyl)-5-methyl-1H-pyrazol-4-yl)-6-(methylamino)picolinate. To a solution of tert-butyl 3-bromo-6-(methylamino)pyridine-2-carboxylate (10 g, 34.3 mmol, Intermediate DB) and 1-(1-adamantylmethyl)-5-methyl-4-(4,4,5,5-tetramethyl-1,3,2-dioxaborolan-2-yl) pyrazole (25.2 g, 70.7 mmol, Intermediate BH) in dioxane (100 mL) and H₂O (40 mL) was added (IS,3R,5R,7S)-1,3,5,7-tetramethyl-8-phenyl-2,4,6-trioxa-8-phosphatricyclo[3.3.1.1^{3,7}]decane (2.07 g, 7.07 mmol, LI), K₃PO₄ (15 g, 70.7 mmol) and Pd₂(dba); (1.62 g, 1.77 mmol). The mixture was degassed and purged with N₂ three times and then it was stirred at 100° C., for 16 hrs. On completion, the reaction was filtered and the residue was diluted with EtOAc (200 mL), washed with water (50 mL×3) and brine (100 mL), dried over Na₂SO₄, filtered and concentrated in vacuo. The crude was purified by silica gel column chromatography: Petroleum: EtOAc from 100:1 to 3:1 to afford the title compound (9.5 g, 20.5 mmol, 62% yield) as a yellow solid. LC-MS (ESI⁺) m/z 437.4 (M+H)⁺.

Step 2—Tert-butyl 3-(1-((1s,3s)-adamantan-1-ylmethyl)-5-methyl-1H-pyrazol-4-yl)-6-((6-(benzo[d]thiazol-2-yl((2-(trimethylsilyl)ethoxy)methyl)amino)-5-methylpyridazin-3-yl)(methyl)amino)picolinate. To a solution of N-(6-chloro-4-methyl-pyridazin-3-yl)-N-(2-trimethylsilylethoxymethyl)-1,3-benzothiazol-2-amine (10 g, 24.6 mmol, Intermediate CP) and tert-butyl 3-[1-(1-adamantylmethyl)-5-methyl-pyrazol-4-yl]-6-(methylamino)pyridine-2-carboxylate (9.5 g, 21.8 mmol) in dioxane (200 mL) was added Pd₂(dba); (1.12 g, 1.23 mmol), Cs₂CO; (16 g, 49.1 mmol) and Xantphos (1.42 g, 2.46 mmol). The mixture was degassed and purged with N₂ 3 times and then it was stirred at 110° C., for 16 hrs. On completion, the reaction was filtered and the residue was diluted with water (600 mL), extracted with EtOAc (200 mL×3), washed with brine (200 mL), dried over Na₂SO₄, filtered and concentrated in vacuo. The crude was purified by silica gel column chromatography: Petroleum: EtOAc from 100:1 to 3:1 to afford the title compound (13 g, 59% yield) as a yellow solid. LC-MS (ESI⁺) m/z 808.1 (M+H)⁺. ¹H NMR (400 MHZ, DMSO-d₆) δ=7.78 (d, J=7.6 Hz, 1H), 7.67-7.60 (m, 2H), 7.49-7.40 (m, 2H), 7.32 (s, 1H), 7.29-7.22 (m, 2H), 5.86 (s, 2H), 3.79-3.68 (m, 4H), 2.36 (s, 3H), 2.18 (s, 3H), 1.93 (br s, 3H), 1.69-1.63 (m, 3H), 1.56 (br s, 10H), 1.35 (s, 9H), 1.08-1.06 (m, 2H), 0.92 (br t, J=8.0 Hz, 2H), −0.11 (s, 9H).

Step 3—3-(1-((1s,3s)-Adamantan-1-ylmethyl)-5-methyl-1H-pyrazol-4-yl)-6-((6-(benzo[d]thiazol-2-ylamino)-5-methylpyridazin-3-yl)(methyl)amino)picolinic acid. To a solution of tert-butyl 3-[1-(1-adamantylmethyl)-5-methyl-pyrazol-4-yl]-6-[6-[1,3-benzothiazol-2-yl (2-trimethylsilylethoxymethyl)amino]-5-methyl-pyridazin-3-yl]-methyl-amino]pyridine-2-carboxylate (13 g, 16.1 mmol) in DCM (5 mL) was added HCl/dioxane (4 M, 11.82 mL). The mixture was stirred at 20° C., for 4 hrs. On completion, a precipitate formed which was filtered and the filter cake was washed with DCM (5 mL×3). The crude was purified by silica gel column chromatography: EtOAc: MeOH from 1:0 to 5:1 to afford the title compound (7 g, 63% yield) as a yellow solid. LC-MS (ESI⁺) m/z 621.2 (M+H)⁺. ¹H NMR (400 MHZ, DMSO-d₆) δ=7.97-7.82 (m, 3H), 7.61-7.51 (m, 2H), 7.49-7.41 (m, 2H), 7.30-7.25 (m, 1H), 3.78 (s, 2H), 3.69 (s, 2H), 3.56 (s, 3H), 2.45 (s, 2H), 2.22 (s, 2H), 1.98-1.90 (m, 3H), 1.70-1.62 (m, 3H), 1.48 (br s, 9H).

### 6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanoic acid (Intermediate FQ)

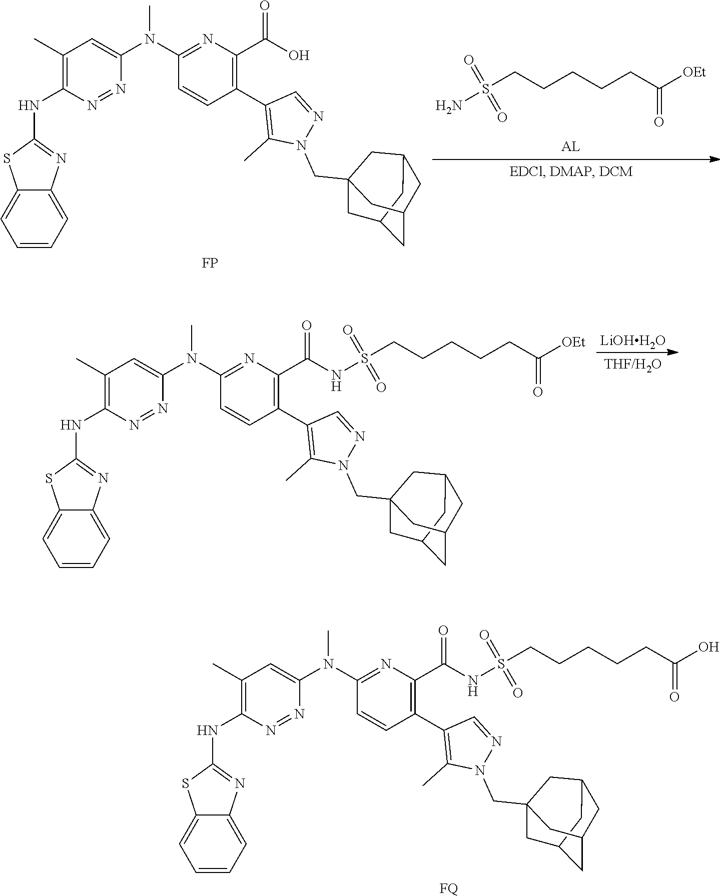

Step 1—Ethyl 6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanoate. To a solution of 3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinic acid (100 mg, 161 umol, Intermediate FP) and ethyl 6-sulfamoylhexanoate (46.7 mg, 209 umol, 5.70 uL, Intermediate AL) in DCM (0.2 mL) was added DMAP (68.8 mg, 563 umol) and EDCI (92.6 mg, 483 umol), then the mixture was stirred at 25° C., for 1 h. On completion, the reaction mixture was quenched with H₂O (5 mL) at 20° C., and extracted with DCM (5 mL×3). The combined organic layers were washed with brine (5 mL×2), dried over Na₂SO₄, filtered and concentrated under reduced pressure to give a residue. The residue was purified by column chromatography (SiO₂, Petroleum ether/Ethyl acetate=3/1 to 0/1) to give the title compound (70 mg, 84.7 umol, 53% yield) as brown solid. LC-MS (ESI⁺) m/z 826.5 (M+1)⁺.

Step 2—6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanoic acid. To a solution of ethyl 6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanoate (70 mg, 84.7 umol) in THF (0.4 mL) and H₂O (0.2 mL) was added LiOH·H₂O (17.7 mg, 423 umol). Then the mixture was stirred at 25° C., for 12 h. On completion, the reaction mixture was quenched with H₂O (20 mL) at 20° C., and extracted with DCM (5 mL×3). The aqueous phase was adjusted to pH=3˜4, and then extracted with DCM (5 mL×3). The organic layers were washed with brine (5 mL×3), dried over Na₂SO₄, filtered and concentrated under reduced pressure to give the title compound (60 mg) as yellow solid. LC-MS (ESI⁺) m/z 798.2 (M+H)⁺.

### 5-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)pentanoic acid (Intermediate FR)

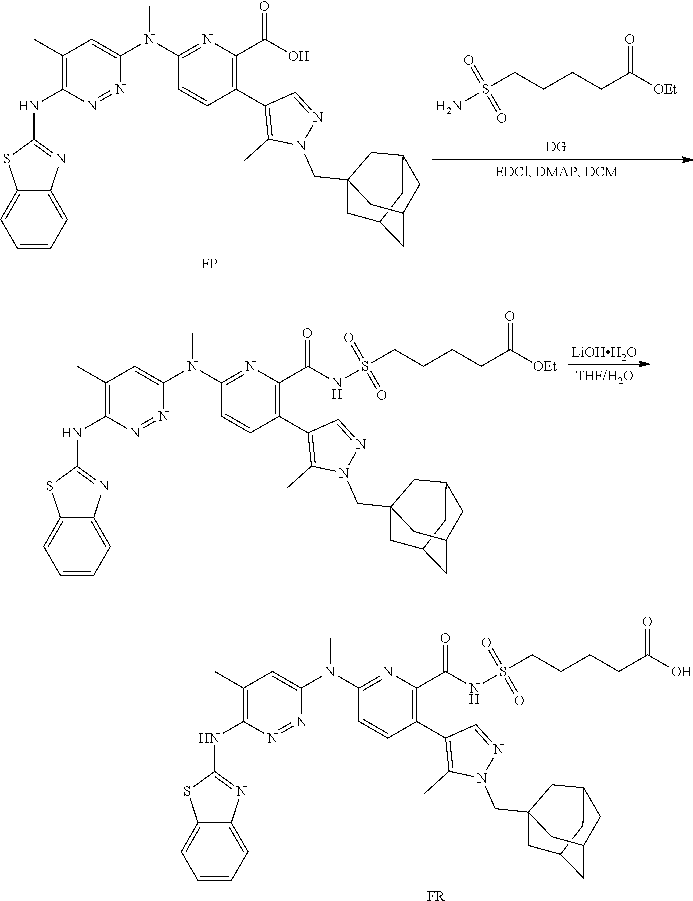

Step 1—Ethyl 5-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)pentanoate. To a solution of 3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carboxylic acid (1.5 g, 2.42 mmol, Intermediate FP) and ethyl 5-sulfamoylpentanoate (758 mg, 3.62 mmol, Intermediate DG) in DCM (15 mL) was added EDCI (1.39 g, 7.25 mmol) and DMAP (1.18 g, 9.67 mmol). The mixture was stirred at 25° C., for 2 hrs. On completion, the reaction mixture was diluted with H₂O (60 mL) and extracted with DCM (50 mL×3). The combined organic layers were dried over Na₂SO₄, filtered and concentrated under reduced pressure to give a residue. The residue was purified by column chromatography (SiO₂, Petroleum ether/Ethyl acetate=I/O to 0/1) to give the title compound (600 mg, 29% yield) as a yellow solid. LC-MS (ESI⁺) m/z 812.4 (M+H)⁺.

Step 2—5-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)pentanoic acid. To a solution of ethyl 5-[[3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carbonyl]sulfamoyl]pentanoate (600 mg, 738 umol) in THF (3 mL) and H₂O (3 mL) was added LiOH·H₂O (310 mg, 7.39 mmol). The mixture was stirred at 25° C., for 12 hrs. On completion, the reaction mixture pH value of the solution was adjusted to 3-5 with HCl (1M), then diluted with H₂O (25 mL) and extracted with EtOAc (30 mL×3). The combined organic layers were dried over Na₂SO₄, filtered and concentrated under reduced pressure to give the title compound (550 mg) as a yellow solid. LC-MS (ESI⁺) m/z 784.6 (M+H)⁺.

### 4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)butanoic acid (Intermediate FS)

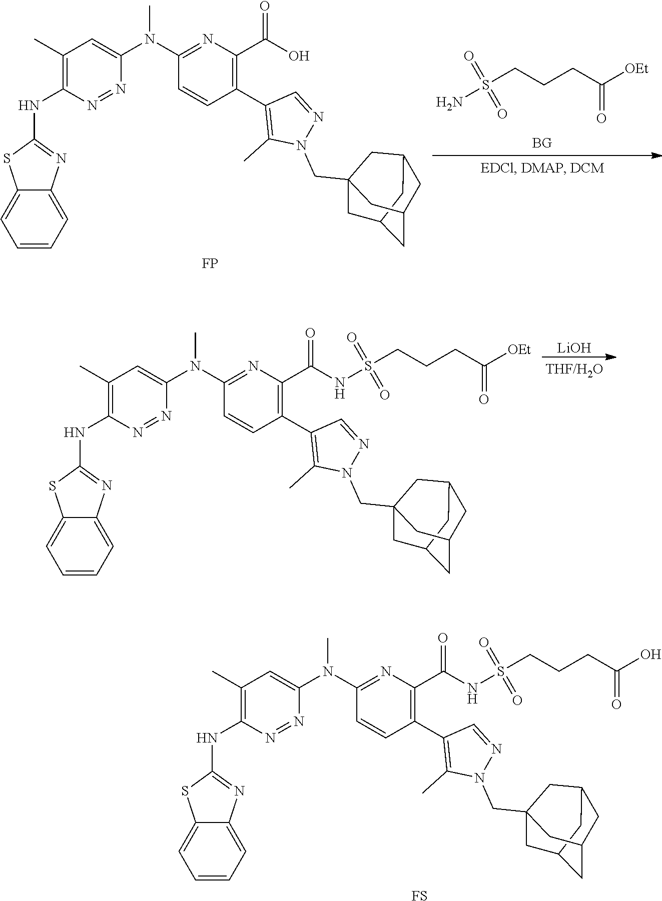

Step 1—Ethyl 4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)butanoate. To a solution of 3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carboxylic acid (0.3 g, 456 umol, Intermediate FP) and ethyl 4-sulfamoylbutanoate (178 mg, 913 umol, Intermediate BG) in DCM (8 mL) was added DMAP (139 mg, 1.14 mmol) and EDCI (175 mg, 913 umol). The mixture was stirred at 25° C., for 16 hrs. On completion, the reaction was diluted with water (20 mL), and extracted with DCM (6 mL×3). The combined organic layer was washed with brine (15 mL), dried over Na₂SO₄, filtered and concentrated in vacuo. The crude was purified by prep-TLC to afford the title compound (40 mg, 9% yield) as a yellow solid. LC-MS (ESI⁺) m/z 798.4 (M+H)⁺.

Step 2—4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)butanoic acid. To a solution of ethyl 4-[[3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carbonyl]sulfamoyl]butanoate (40 mg, 50.1 umol) in THF (0.5 mL) and H₂O (0.2 mL) was added LiOH·H₂O (10.5 mg, 251 umol). The mixture was stirred at 25° C., for 3 hrs. On completion, the reaction was diluted with concentrated in vacuo to afford the title compound (35 mg) as a yellow solid. LC-MS (ESI⁺) m/z 770.1 (M+H)⁺.

### 5-bromopentane-1-sulfonamide (Intermediate FT)

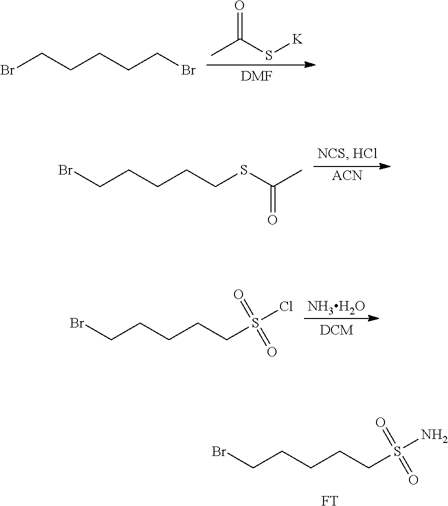

Step 1—S-(5-bromopentyl)ethanethioate. A mixture of 1,5-dibromopentane (10.0 g, 43.4 mmol, 5.88 mL, CAS #111-24-0) and K₂CO₃ (12.0 g, 87.0 mmol) in DMF (100 mL) was added acetylsulfanylpotassium (4.72 g, 41.3 mmol) dropwise at 0° C., under N₂, then the mixture was stirred at 25° C., for 2 h. On completion, the reaction mixture was poured into water (100 mL), and then extracted with EtOAc (100 mL×2). The combined organic layers were washed with brine (100 mL×3), dried over Na₂SO₄, filtered and concentrated under reduced pressure to give a residue. The residue was purified by column chromatography (SiO₂, Petroleum ether/Ethyl acetate=100/1-10/1). To give the title compound (4.20 g, 43% yield) as a colorless oil. ¹H NMR (400 MHz, CHLOROFORM-d) δ=3.40 (t, J=6.8 Hz, 2H), 2.88 (t, J=7.2 Hz, 2H), 2.33 (s, 3H), 1.88 (quin, J=7.2 Hz, 2H), 1.65-1.56 (m, 2H), 1.56-1.47 (m, 2H).

Step 2—5-Bromopentane-1-sulfonyl chloride. To a solution of S-(5-bromopentyl) ethanethioate (4.20 g, 18.6 mmol) in MeCN (42 mL) was added HCl (2 M, 13.0 mL) and NCS (9.96 g, 74.6 mmol) at 0° C. The mixture was stirred at 20 for 0.5 h. On completion, the mixture was poured into H₂O (50 mL), and extracted with EtOAc mL (50 mL×3). The combined organic layer was washed with brine (50 mL×2), dried over anhydrous Na₂SO₄, filtered and concentrated under reduced pressure to give the title compound (4.50 g, 97% yield) as a white solid.

Step 3—5-bromopentane-1-sulfonamide. To a solution of 5-bromopentane-1-sulfonyl chloride (4.50 g, 18.0 mmol) in DCM (25 mL) was added NH₃·H₂O (50.5 g, 360 mmol, 55.5 mL, 25% solution) at 0° C. The mixture was then stirred at 25° C., for 1 h. On completion, the reaction mixture was washed with NaHCO₃ (20 mL×3), organic layer was separated and washed with brine (10 mL×3), dried over Na₂SO₄, filtered and concentrated under reduced pressure to give a residue. The residue was purified by column chromatography (SiO₂, Petroleum ether/Ethyl acetate=20/1-1/1) to give the title compound (1.00 g, 24% yield) as a white solid. ¹H NMR (400 MHZ, CDCl₃) δ=4.74 (br s, 2H), 3.43 (t, J=6.4 Hz, 2H), 3.19-3.11 (m, 2H), 1.96-1.87 (m, 4H), 1.63 (s, 2H).

### [1-[(4-Methoxyphenyl) methyl]-2,6-dioxo-3-piperidyl]trifluoromethanesulfonate (Intermediate FU)

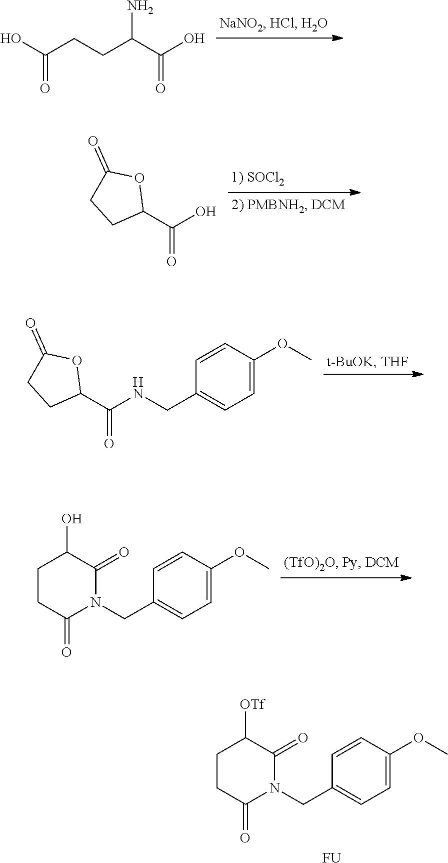 [SMILES: NC(CCC(=O)O)C(=O)O ; O=C1CCC(C(=O)O)O1 ; COc1ccc(CNC(=O)C2CCC(=O)O2)cc1 ; COc1ccc(CN2C(=O)CCC(O)C2=O)cc1 ; COc1ccc(CN2C(=O)CCC(OS(=O)(=O)C(F)(F)F)C2=O)cc1 ; "FU"]

Step 1—5-Oxotetrahydrofuran-2-carboxylic acid. To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at −5° C. The mixture was stirred at 15° C., for 12 hrs. On completion, the mixture was concentrated and then dissolved in EA (500 mL) and filtered and washed with EA (3×100 mL). The filtrate and washed solution were dried over Na₂SO₄, filtered and concentrated in vacuo to give the title compound (200 g, crude) as yellow oil. ¹H NMR (400 MHZ, CDCl₃) δ 6.43 (s, 1H), 5.02-4.95 (m, 1H), 2.67-2.38 (m, 4H).

Step 2—N-[(4-methoxyphenyl)methyl]-5-oxo-tetrahydrofuran-2-carboxamide. To 5-oxotetrahydrofuran-2-carboxylic acid (120 g, 922 mmol) was added SOCl₂ (246 g, 2.07 mol) at 0° C., slowly. The mixture was stirred at 85° C., for 3 hrs, and then the mixture was stirred at 15° C., for 6 hrs. The mixture was concentrated in vacuo. The residue was dissolved in dry DCM (1 L) at 0° C., under N₂. After that a solution of Et N (187 g, 1.84 mol) and 4-methoxybenzylamine (101 g, 738 mmol) in DCM (400 mL) was added, then the mixture was stirred at 15° C., for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHZ, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

Step 3—3-Hydroxy-1-[(4-methoxyphenyl)methyl]piperidine-2,6-dione. A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C., under nitrogen atmosphere. The resulting reaction mixture was stirred at −40° C., for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

Step 4—[1-[(4-Methoxyphenyl)methyl]-2,6-dioxo-3-piperidyl]trifluoromethanesulfonate. To a solution of 3-hydroxy-1-[(4-methoxyphenyl)methyl]piperidine-2,6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C., for 1.5 hours under N₂. On completion, the mixture was concentrated in vacuo. The residue was purified by column chromatography on silica gel (PE:EA=20:1/8:1) to give the title compound (45.0 g, 68% yield) as light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### 3-(4-Bromo-3-methyl-2-oxo-2,3-dihydro-1H-benzo[d]imidazol-1-yl)piperidine-2,6-dione (Intermediate FV)

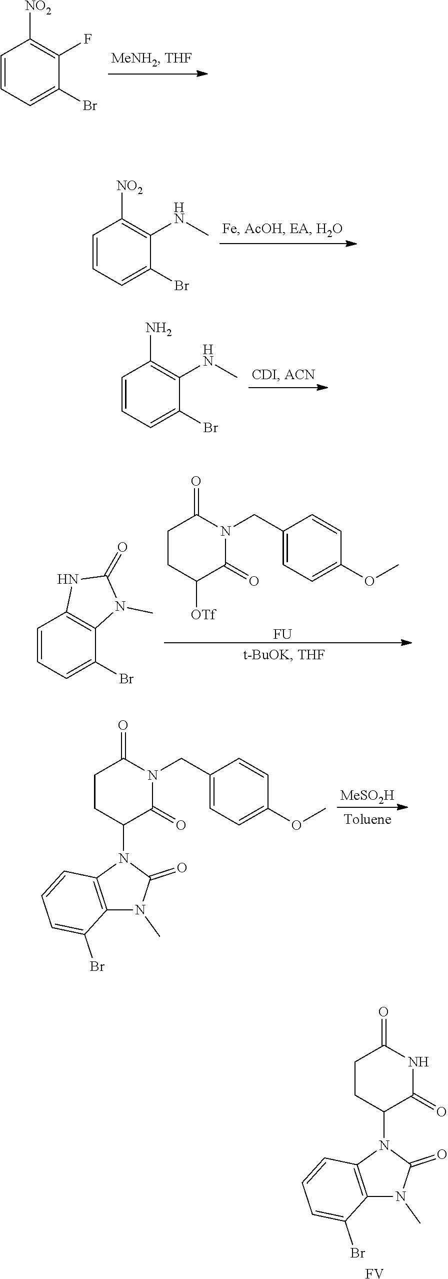

Step 1—2-Bromo-N-methyl-6-nitro-aniline. To a solution of 1-bromo-2-fluoro-3-nitro-benzene (40.0 g, 181 mmol, CAS #58534-94-4) in THF (40 mL) was added MeNH₂ (2 M, 400 mL). The reaction mixture was stirred at 60° C., for 12 hours. On completion, the reaction mixture was poured into sat. NaHCO; (30 mL) and extracted with EA (3×200 mL). The combined organic layers were washed with brine (2×200 mL), dried with anhydrous Na₂SO₄, filtered and concentrated in vacuo to give the title compound (40.0 g, 95% yield) as red oil. LC-MS (ESI⁺) m/z 230.9 (M+H)⁺.

Step 2—3-Bromo-N₂-methyl-benzene-1,2-diamine. To a mixture of 2-bromo-N-methyl-6-nitro-aniline (23.0 g, 99.5 mmol) in EA (300 mL) and H₂O (10 mL) was added AcOH (100 mL). The mixture was warmed to 50° C. Then Fe (22.2 g, 398 mmol) was added to the reaction mixture and the mixture was heated to 80° C., about 4 hours. On completion, the reaction mixture was filtered and concentrated in vacuo. The residue was diluted with water (100 mL) and extracted with EA (3×200 mL). The combined organic layers was dried over Na₂SO₄, filtered and concentrated in vacuo to give the title compound (20.0 g, 99% yield) as red oil. ¹H NMR (400 MHZ, DMSO-d₆) δ 6.73-6.70 (m, 1H), 6.68-6.60 (m, 2H), 5.02 (s, 2H), 3.67 (s, 1H), 2.58 (s, 3H).

Step 3—4-Bromo-3-methyl-1H-benzimidazol-2-one. To a mixture of 3-bromo-N₂-methyl-benzene-1,2-diamine (20.0 g, 99.4 mmol) in ACN (300 mL) was added CDI (32.2 g, 198 mmol). The reaction mixture was stirred at 85° C., for 12 hours under N₂ atmosphere. On completion, the reaction mixture was concentrated in vacuo. The reaction mixture was diluted with water (200 mL), where a solid precipitate was formed, which was filtered off. The solid was washed with water (1 L) and dried in vacuo to give the title compound (20.0 g, 88% yield) as white solid. ¹H NMR (400 MHZ, DMSO-d₆) δ 11.17 (s, 1H), 7.14 (dd, J=1.2, 8.0 Hz, 1H), 7.00-6.95 (m, 1H), 6.93-6.87 (m, 1H), 3.55 (s, 3H).

Step 4—3-(4-Bromo-3-methyl-2-oxo-benzimidazol-1-yl)-1-](4-methoxyphenyl)methyl]piperidine-2,6-dione. To a solution of 4-bromo-3-methyl-1H-benzimidazol-2-one (12.0 g, 52.8 mmol) in THF (300 mL) was added t-BuOK (7.12 g, 63.4 mmol). The reaction mixture was stirred at 0° C., for 0.5 hr. Subsequently, [1-[(4-methoxyphenyl)methyl]-2,6-dioxo-3-piperidyl]trifluoromethanesulfonate (20.1 g, 52.8 mmol, Intermediate FU) in a solution of THF (100 mL) was added dropwise. The resulting reaction mixture was stirred at 20° C., for 0.5 hr under N₂. On completion, the reaction mixture was quenched with saturated NH₄Cl (100 mL), and extracted with ethyl acetate (200 mL). The combined organic layers were washed with brine (2×100 mL), dried over anhydrous sodium sulfate, filtered, the filtrate was concentrated in vacuo. The crude product was purified by reversed-phase HPLC (0.1% FA condition) to give the title compound (13.3 g, 55% yield) as a yellow solid. ¹H NMR (400 MHZ, CDCl₃) δ 7.38 (d, J=8.8 Hz, 2H), 7.22 (d, J=8.0 Hz, 1H), 6.84 (d, J=8.8 Hz, 2H), 6.80 (t, J=8.0 Hz, 1H), 6.48-6.40 (d, J=8.0 Hz, 1H), 5.22 (dd, J=5.2, 12.8 Hz, 1H), 5.04-4.93 (m, 2H), 3.81 (s, 3H), 3.80 (s, 3H), 3.12-2.98 (m, 1H), 2.93-2.77 (m, 1H), 2.62 (dq, J=4.4, 13.2 Hz, 1H), 2.20-2.17 (m, 1H).

Step 5—3-(4-bromo-3-methyl-2-oxo-2,3-dihydro-1H-benzo[d]imidazol-1-yl)piperidine-2,6-dione. A mixture of 3-(4-bromo-3-methyl-2-oxo-benzimidazol-1-yl)-1-](4-methoxyphenyl)methyl]piperidine-2,6-dione (13.3 g, 29.0 mmol) in a mixed solvent of Tol. (80 mL) and methane sulfonic acid (40 mL) was degassed and purged with N₂ for 3 times, and then the mixture was stirred at 120° C., for 2 hrs under N₂ atmosphere. On completion, the reaction mixture was concentrated in vacuo to remove toluene. The residue was added 200 mL of ice water, and then white solid precipitate formed. The mixture was filtered and the filtered cake was collected and dried over in vacuo to give the title compound (7.30 g, 74% yield) as white solid. ¹H NMR (400 MHZ, DMSO-d₆) δ 11.13 (s, 1H), 7.25 (d, J=8.0 Hz, 1H), 7.17 (d, J=8.0 Hz, 1H), 7.05-6.93 (m, 1H), 5.41 (dd, J=5.2, 12.8 Hz, 1H), 3.64 (s, 3H), 2.96-2.83 (m, 1H), 2.78-2.59 (m, 2H), 2.08-2.00 (m, 1H)

### 5-(1-(2,6-Dioxopiperidin-3-yl)-3-methyl-2-oxo-2,3-dihydro-1H-benzo[d]imidazol-4-yl) pentane-1-sulfonamide (Intermediate FW)

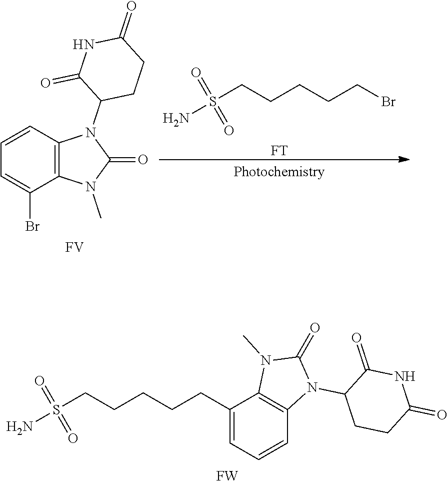

To a 15 mL vial equipped with a stir bar was added 5-bromopentane-1-sulfonamide (300 mg, 1.30 mmol, Intermediate FT), 3-(4-bromo-3-methyl-2-oxo-benzimidazol-1-yl)piperidine-2,6-dione (573 mg, 1.69 mmol, Intermediate FV), Ir[dF (CF3)ppy]₂(dtbpy)(PF6) (29.2 mg, 26.0 umol), NiCl₂.dtbbpy (15.5 mg, 39.1 umol), TTMSS (324 mg, 1.30 mmol, 402 uL), and 2,6-Lutidine (279 mg, 2.61 mmol, 303 uL) in DME (30 mL). The vial was sealed and placed under nitrogen. The reaction was stirred and irradiated with a 10 W blue LED lamp (3 cm away), with cooling water to keep the reaction temperature at 25° C., for 14 h. On completion, the mixture was poured into with H₂O (5 mL), and extracted with DCM mL (5 mL×3). The combined organic layer was washed with brine (5 mL), dried over anhydrous Na₂SO₄, filtered and concentrated under reduced pressure to give a residue. The residue was purified by column chromatography (SiO₂, Petroleum ether:Ethyl acetate=5:1 to Petroleum ether:Ethyl acetate=0:1) to give the title compound (250 mg, 47% yield) as a yellow solid. LC-MS (ESI⁺) m/z 409.2 (M+H)⁺. 5-Bromo-3-methyl-1H-benzimidazol-2-one (Intermediate FX)

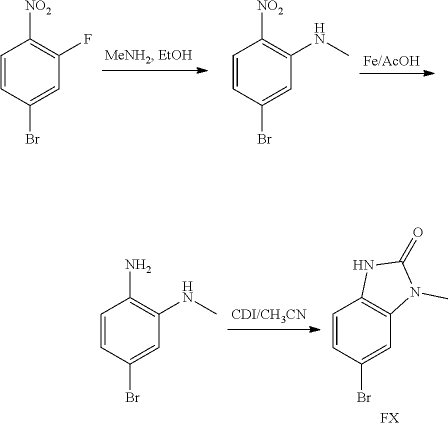

Step 1—5-Bromo-N-methyl-2-nitro-aniline. 4-bromo-2-fluoro-1-nitro-benzene (230 g, 1.05 mol, CAS #321-23-3) was added to a solution of methylamine in tetrahydrofuran (2 M, 1.51 L). The mixture was stirred at 15° C., for 10 minutes. On completion, the mixture was diluted with H₂O (250 mL) and extracted with EtOAc (3×300 mL). The combined organic layers were washed with brine (300 mL), dried over Na₂SO₄, filtered and concentrated in vacuo to give the title compound (200 g, 83% yield) as a yellow solid. ¹H NMR (400 MHz, DMSO-d₆) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

Step 2—4-Bromo-N₂-methyl-benzene-1,2-diamine. To a mixture of 5-bromo-N-methyl-2-nitro-aniline (200 g, 865 mmol) in EtOAc (1 L) and H₂O (500 mL) was added AcOH (1.00 L). The mixture was warmed to 50° C., and then Fe (174 g, 3.11 mol) was added to the reaction mixture. After that, the reaction mixture was stirred at 80° C., for 6 hours. On completion, the mixture was filtered through celite. The filtrate was concentrated in vacuo and the residue was diluted with H₂O (250 mL) and extracted with EtOAc (3×300 mL). The combined organic layers were washed with aq. NaHCO; and brine (300 mL), dried over Na₂SO₄, filtered and concentrated in vacuo. The residue was purified by flash silica gel chromatography to give the title compound (130 g, 75% yield) as black oil. ¹H NMR (400 MHZ, DMSO-d₆) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

Step 3—5-Bromo-3-methyl-1H-benzimidazol-2-one. To a solution of 4-bromo-N₂-methyl-benzene-1,2-diamine (110 g, 547 mmol) in CH₃CN (1.3 L) was added CDI (177 g, 1.09 mol). The mixture was stirred at 80° C., for 6 hours under N₂. On completion, the mixture was concentrated in vacuo. The mixture was diluted with H₂O (1.0 L) and filtered. The filter cake was washed with water (3×200 mL) and dried in vacuo to give the title compound (106 g, 85% yield) as a white solid. ¹H NMR (400 MHZ, DMSO-d₆) δ 11.00 (s, 1H), 7.33 (s, 1H), 7.13 (d, J=8.0 Hz, 1H), 6.92 (d, J=8.0 Hz, 1H), 3.27 (s, 3H).

### 3-(5-bromo-3-methyl-2-oxo-benzimidazol-1-yl)piperidine-2,6-dione (3-(5-bromo-3-methyl-2-oxo-2,3-dihydro-1H-1,3-benzodiazol-1-yl)piperidine-2,6-dione) (Intermediate FY)

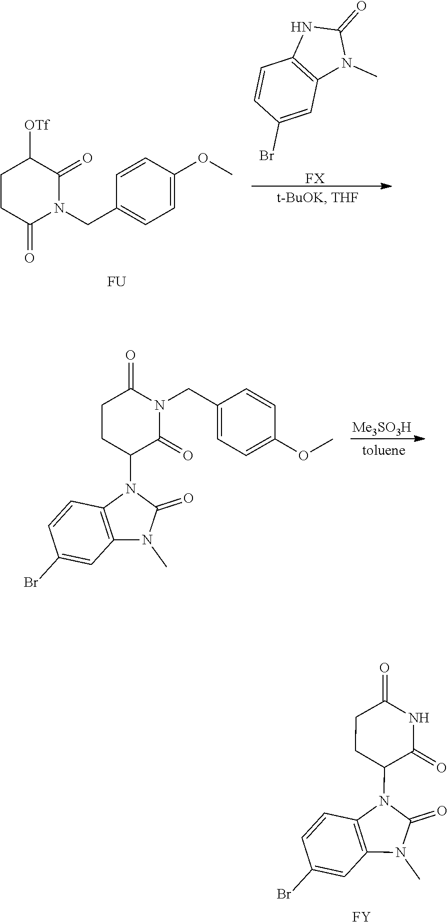

Step 1—3-(5-Bromo-3-methyl-2-oxo-benzimidazol-1-yl)-1-[(4-methoxyphenyl)methyl]piperidine-2,6-dione. To a solution of 5-bromo-3-methyl-1H-benzimidazol-2-one (4.90 g, 21.6 mmol, Intermediate FX) in THF (300 mL) was added t-BuOK (3.63 g, 32.3 mmol) at 0° C. The mixture was stirred at 0-10° C., for 1 hour under N₂. Then a solution of [1-[(4-methoxyphenyl)methyl]-2,6-dioxo-3-piperidyl]trifluoromethanesulfonate (9.87 g, 25.9 mmol, Intermediate FU) in THF (100 mL) was added to the reaction mixture at 0-10° C., during 30 minutes. The mixture was stirred at 0-10° C., for 30 minutes under N₂. An additional solution of [1-[(4-methoxyphenyl)methyl]-2,6-dioxo-3-piperidyl]trifluoromethanesulfonate (2.47 g, 6.47 mmol) in THF (20 mL) was added to the reaction mixture at 0-10° C. dropwise. The mixture was then stirred at 0-10° C., for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. ¹H NMR (400 MHZ, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

Step 2—3-(5-Bromo-3-methyl-2-oxo-benzimidazol-1-yl)piperidine-2,6-dione. To a mixture of 3-(5-bromo-3-methyl-2-oxo-benzimidazol-1-yl)-1-[(4-methoxyphenyl)methyl]piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C., for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### 5-(1-(2,6-dioxopiperidin-3-yl)-3-methyl-2-oxo-2,3-dihydro-1H-benzo[d]imidazol-5-yl) pentane-1-sulfonamide (Intermediate FZ)

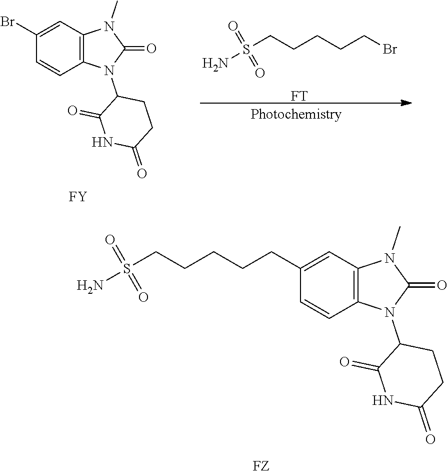

To a 15 mL vial equipped with a stir bar was added 3-(5-bromo-3-methyl-2-oxo-2,3-dihydro-1H-benzo[d]imidazol-1-yl)piperidine-2,6-dione (339 mg, 1.00 mmol, Intermediate FY), 5-bromopentane-1-sulfonamide (300 mg, 1.30 mmol, Intermediate FT), Ir[dF(CF₃)ppy]₂(dtbpy) (PF6) (11.3 mg, 10.0 umol), NiCl₂·dtbbpy (5.99 mg, 15.0 umol), TTMSS (249 mg, 1.00 mmol, 309 uL), and 2,6-lutidine (215 mg, 2.01 mmol, 234 uL) in DME (30 mL). The vial was sealed and placed under nitrogen. The reaction was then stirred and irradiated with a purple 10 W LED lamp (3 cm away), with cooling water to keep the reaction temperature at 25° C., for 14 hr. On completion, the mixture was poured into with H₂O (20 mL), and extracted with DCM mL (20 mL×3). The combined organic layer was washed with brine (20 mL), dried over anhydrous Na₂SO₄, filtered and concentrated under reduced pressure to give a residue. The residue was purified by column chromatography (SiO₂, Petroleum ether:Ethyl acetate=5:1 to Petroleum ether:Ethylacetate=0:1) to afford the title compound (260 mg, 64% yield) as a white solid. LC-MS (ESI⁺) m z 409.1 (M+H)⁺.

### N,N-bis(4-methoxybenzyl)ethenesulfonamide (Intermediate GA)

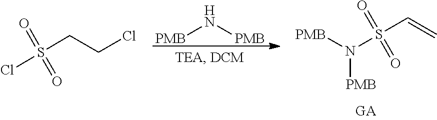

To a solution of 1-(4-methoxyphenyl)-N-[(4-methoxyphenyl)methyl]methanamine (10.0 g, 38.9 mmol, CAS #17061-62-0) in DCM (100 mL) was added TEA (13.8 g, 136 mmol, 18.9 mL) and a solution of 2-chloroethanesulfonyl chloride (6.97 g, 42.8 mmol, 4.47 mL, CAS #1622-32-8) in DCM (60 mL) at 0° C. After addition, the mixture was stirred at 0° C., for 2 hrs. On completion, the reaction mixture was quenched with H₂O (10 mL) at 0° C., and then diluted with H₂O (40 mL) and extracted with DCM (30 mL×3). The combined organic layers were washed dried over Na₂SO₄, filtered to give the filtrate and concentrated under reduced pressure to give a residue. The residue was purified by column chromatography (SiO₂, Petroleum ether/Ethyl acetate=I/O to 3/1) to give the title compound (6.30 g, 47% yield) as a white solid. LC-MS (ESI⁺) m/z 370.0 (M+Na)⁺; ¹H NMR (400 MHZ, DMSO-d₆) δ=7.16 (d, J=8.4 Hz, 4H), 6.88 (d, J=8.4 Hz, 4H), 6.72 (dd, J=10.0, 16.4 Hz, 1H), 6.11 (d, J=16.4 Hz, 1H), 6.03 (d, J=10.0 Hz, 1H), 4.13 (s, 4H), 3.73 (s, 6H).

### Benzyl 3-(methyl (2-sulfamoylethyl)amino)propanoate (Intermediate GB)

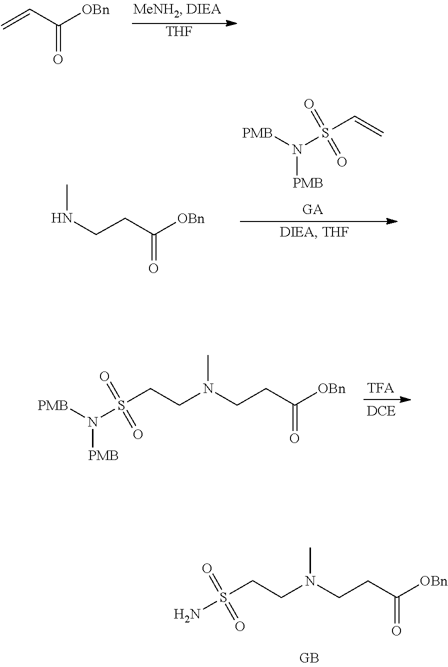

Step 1—Benzyl 3-(methylamino)propanoate. To a solution of benzyl prop-2-enoate (3.00 g, 18.5 mmol, CAS #2495-35-4), DIEA (7.17 g, 55.5 mmol, 9.67 mL) in THF (20 mL), was added methanamine (2 M, 13.9 mL), the mixture was stirred at 60° C., for 12 h. On completion, the reaction mixture was concentrated under reduced pressure to give a residue. The residue was purified by flash silica gel chromatography (ISCOR: 40 g SepaFlash® Silica Flash Column, Eluent of 0˜100% Ethyl acetate/Petroleum ether gradient (a, 80 mL/min) to afford the title compound (2.00 g, 55.9% yield) as a yellow solid. ¹H NMR (400 MHZ, DMSO-d₆)=7.40-7.30 (m, 4H), 5.09 (s, 2H), 3.37-3.25 (m, 1H), 2.73-2.68 (m, 2H), 2.49-2.45 (m, 2H), 2.24 (s, 3H).

Step 2—Benzyl 3-((2-(N,N-bis(4-methoxy benzyl)sulfamoyl)ethyl)(methyl)amino)propanoate. To a solution of benzyl 3-(methylamino)propanoate (1.84 g, 9.50 mmol) and N,N-bis(4-methoxy benzyl)ethenesulfonamide (3.00 g, 8.63 mmol, Intermediate GA) in THF (30 mL), was added DIEA (3.35 g, 25.9 mmol, 4.51 mL), then the mixture was stirred at 60° C., for 12 h. On completion, the reaction mixture was poured into ice water (30 mL) and extracted with ethyl acetate (60 mL×3). The combined organic layers were washed with brine (60 mL×3), dried over sodium sulfate, filtered and concentrated under reduced pressure to give a residue. The residue was purified by flash silica gel chromatography (ISCO®; 20 g SepaFlash®; Silica Flash Column, Eluent of 0˜60% Ethyl acetate/Petroleum ether gradient @80 mL/min) to afford the title compound (2.00 g, 42.84% yield) as a white oil. ¹H NMR (400 MHZ, DMSO-d₆)=7.39-7.30 (m, 5H), 7.18 (d, J=8.8 Hz, 4H), 6.88 (d, J=8.8 Hz, 4H), 5.08 (s, 2H), 4.20 (s, 4H), 3.72 (s, 6H), 3.16-3.10 (m, 2H), 2.70-2.64 (m, 2H), 2.60-2.56 (m, 2H), 2.48-2.44 (m, 2H), 2.10 (s, 3H).

Step 3—Benzyl 3-(methyl (2-sulfamoylethyl)amino)propanoate. To a solution of benzyl 3-((2-(N,N-bis(4-methoxybenzyl)sulfamoyl)ethyl)(methyl)amino)propanoate (1.5 g, 2.77 mmol) in DCE (7.5 mL) was added TFA (316 mg, 2.77 mmol, 7.5 mL). The mixture was stirred at 70° C., for 12 h. On completion, the reaction mixture was concentrated under reduced pressure to give a residue. The crude product was purified by reversed-phase HPLC (0.1% NH₃·H₂O condition) to afford the title compound (378 mg, 45% yield) as a yellow solid. ¹H NMR (400 MHZ, DMSO-d₆) δ=7.44-7.29 (m, 5H), 6.74 (s, 2H), 5.09 (s, 2H), 3.13-3.07 (m, 2H), 2.78-2.72 (m, 2H), 2.67-2.60 (m, 2H), 2.54-2.51 (m, 2H), 2.17 (s, 3H).

### 3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)(tert-butoxycarbonyl)amino)propanoic acid (Intermediate GC)

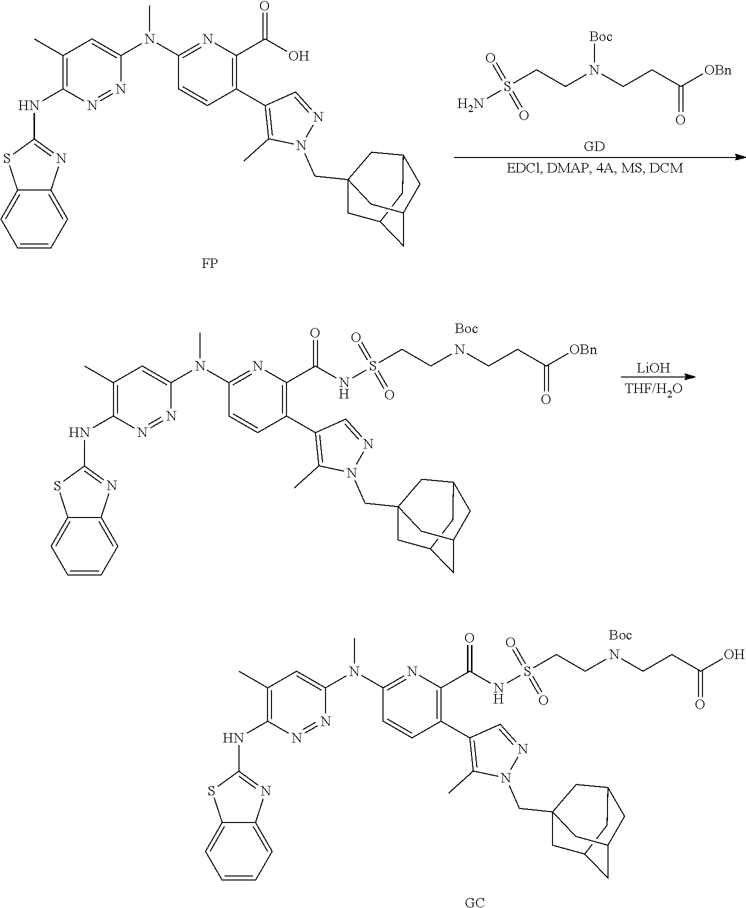

Step 1—Benzyl 3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl) (tert-butoxycarbonyl)amino)propanoate. To a solution of 3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carboxylic acid (1.00 g, 1.61 mmol, Intermediate FP), benzyl 3-[tert-butoxycarbonyl (2-sulfamoylethyl)amino]propanoate (934 mg, 2.42 mmol, Intermediate GD) in DCM (10 mL) was added EDCI (618 mg, 3.22 mmol), DMAP (787 mg, 6.44 mmol) and 4A molecular sieves (1.00 g). The mixture was stirred at 25° C., for 12 h. On completion, the reaction was poured into water (10 mL) and extracted with DCM (10 mL×3). The combined organic phase is washed with brine (5 mL×2), dried over Na₂SO₄, then filtered to get the filtrate and concentrated to give a residue. The residue was purified by prep-HPLC (TFA condition) to afford the title compound (150 mg, 9% yield) as a white solid. LC-MS (ESI⁺) m/z 990.2 (M+H)⁺.

Step 2—3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl) (tert-butoxycarbonyl)amino)propanoic acid. To a solution of benzyl 3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl) (tert-butoxycarbonyl)amino)propanoate (150 mg, 152 umol) in THF (1.5 mL) and H₂O (0.5 mL), was added LiOH·H₂O (31.8 mg, 758 umol). The mixture was stirred at 25° C., for 12 h. On completion, the reaction mixture was poured into water (2 mL) and extracted with DCM (4 mL×3). The aqueous phase was then acidified with HCl (2M) to pH=4-5, and extracted with DCM (4 mL×3). The combined organic layers were washed with brine (5 mL×3), dried over Na₂SO₄, filtered and concentrated under reduced pressure to give the title compound (130 mg, 95% yield) as a yellow solid. LC-MS (ESI⁺) m/z 899.5 (M+H)⁺.

### Benzyl 3-[tert-butoxycarbonyl (2-sulfamoylethyl)amino]propanoate (Intermediate GD)

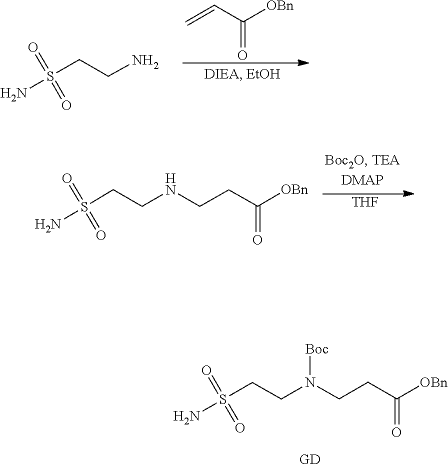 [SMILES: NCCS(N)(=O)=O ; C=CC(=O)OCc1ccccc1 ; NS(=O)(=O)CCNCCC(=O)OCc1ccccc1 ; CC(C)(C)OC(=O)N(CCC(=O)OCc1ccccc1)CCS(N)(=O)=O ; "THF"]

Step 1—Benzyl 3-((2-sulfamoylethyl)amino)propanoate. To a solution of 2-aminoethanesulfonamide (20.0 g, 125 mmol, HCl, CAS #4378-70-5) and benzyl prop-2-enoate (22.21 g, 137 mmol, CAS #2495-35-4) in EtOH (200 mL) was added DIEA (48.3 g, 374 mmol, 65.1 mL). Then the mixture was stirred at 60° C., for 12 h. On completion, the reaction mixture was poured into ice water (200 mL) and extracted with EtOAc (400 mL×3). The combined organic layers were washed with brine (400 mL×3), dried over sodium sulfate, filtered and concentrated under reduced pressure to give a residue. The residue was purified by column chromatography (SiO₂, Petroleum ether/Ethyl acetate=I/O to 1/1) to afford the title compound (13.0 g, 37% yield) as a white solid. ¹H NMR (400 MHZ, DMSO-d₆) δ=7.46-7.29 (m, 5H), 6.78 (br s, 2H), 5.10 (s, 2H), 3.11-3.06 (m, 2H), 2.91-2.86 (m, 2H), 2.80-2.75 (m, 2H), 2.49-2.46 (m, 2H).

Step 2—Benzyl 3-((tert-butoxycarbonyl)(2-sulfamoylethyl)amino)propanoate. To a solution of benzyl 3-((2-sulfamoylethyl)amino)propanoate (2.00 g, 6.98 mmol), TEA (1.41 g, 13.9 mmol, 1.94 mL) and DMAP (427 mg, 3.49 mmol) in THF (20 mL), was added Boc₂O (1.83 g, 8.38 mmol, 1.93 mL) at 0° C., then the mixture was stirred at 25° C., for 4 h. On completion, the reaction mixture was poured into NH₄Cl (sat, aq, 20 mL) and extracted with EtOAc (20 mL×3). The combined organic layers were washed with brine (20 mL×3), dried over sodium sulfate, filtered and concentrated under reduced pressure to give a residue. The crude product was purified by reversed-phase HPLC (0.1% FA condition) to give a residue (55.0 mg, 55% yield) as a white oil. ¹H NMR (400 MHZ, DMSO-d₆) δ 7.42-7.31 (m, 5H), 6.90 (br s, 2H), 5.09 (br s, 2H), 3.55-3.48 (m, 2H), 3.44 (t, J=7.2 Hz, 2H), 3.21-3.15 (m, 2H), 2.60 (br s, 2H), 1.38 (s, 9H).

### 3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)(methyl)amino)propanoic acid (Intermediate GE)

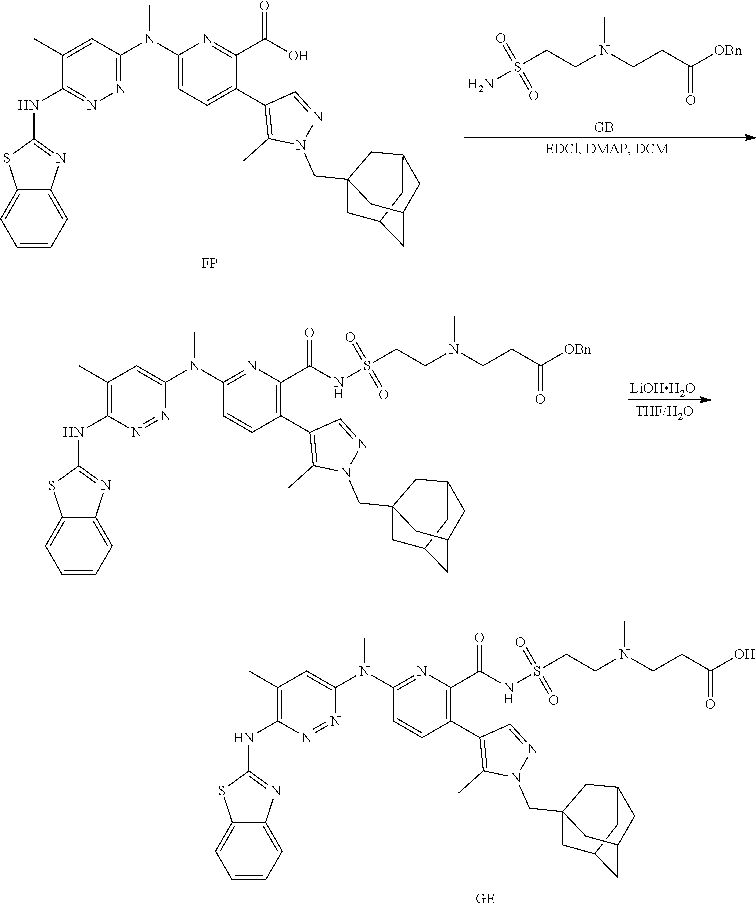

Step 1—Benzyl 3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)(methyl)amino)propanoate. To a solution of 3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinic acid (180 mg, 278 umol, HCl, Intermediate FP) and benzyl 3-(methyl (2-sulfamoylethyl)amino)propanoate (329 mg, 1.10 mmol, Intermediate GB) in DCM (4 mL), was added EDCI (105 mg, 548 umol), DMAP (134 mg, 1.10 mmol), 4 Å molecular sieves (10.0 mg, 273 umol), then the mixture was stirred at 25° C., for 12 h. On completion, the reaction mixture was poured into ice water (5 mL) and extracted with DCM (5 mL×3). The combined organic layers were washed with brine (5 mL×3), dried over sodium sulfate, filtered and concentrated under reduced pressure to give a residue. The crude product was purified by reversed-phase HPLC (0.1% FA condition) to afford the title compound (90.0 mg, 36% yield) as a yellow solid. LC-MS (ESI⁺) m/z 903.4 (M+H)⁺.

Step 2—3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)(methyl)amino)propanoic acid. To a solution of benzyl 3-((2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)(methyl)amino)propanoate (90.0 mg, 99.6 umol) in THF (0.8 mL) and H₂O (0.2 mL) was added LiOH·H₂O (20.9 mg, 498 umol), then the mixture was stirred at 25° C., for 12 h. On completion, the reaction mixture was poured into water (2 mL) and extracted with DCM (2 mL×3). The aqueous phase was acidified with IM HCl to pH=4, then extracted with DCM (4 mL×3). The combined organic layers were washed with brine (5 mL×3), dried over Na₂SO₄, filtered and concentrated under reduced pressure to give the title compound (80.0 mg) as a yellow solid. LC-MS (ESI⁺) m/z 813.5 (M+H)⁺.

### Ethyl 3-(methyl (2-sulfamoylethyl)amino)propanoate (Intermediate GF)

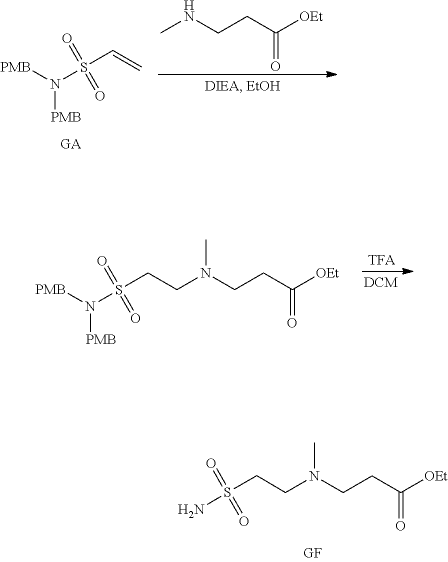

Step 1—Ethyl 3-((2-(N,N-bis(4-methoxybenzyl)sulfamoyl)ethyl)(methyl)amino)propanoate. To a solution of N,N-bis[(4-methoxyphenyl)methyl]ethenesulfonamide (2.78 g, 8.00 mmol, Intermediate GA) in EtOH (200 mL) was added DIEA (4.14 g, 32.0 mmol, 5.58 mL) and ethyl 3-(methylamino)propanoate (2.1 g, 16.0 mmol, CAS #2213 Aug. 3). The mixture was then stirred at 60° C., for 12 hrs. On completion, the reaction mixture was concentrated under reduced pressure to remove EtOH. The mixture was purified by column chromatography (SiO₂, Petroleum ether/Ethyl acetate=I/O to 1/1) to give the title compound (2.38 g, 4.91 mmol, 61% yield) as a light-yellow oil. LC-MS (ESI⁺) m/z 479.4 (M+H)⁺; ¹H NMR (400 MHZ, DMSO-d₆) δ=7.19 (d, J=8.8 Hz, 4H), 6.91-6.86 (m, 4H), 4.20 (s, 4H), 4.03 (q, J=7.2 Hz, 2H), 3.73 (s, 6H), 3.16-3.11 (m, 2H), 2.69-2.63 (m, 2H), 2.57-2.52 (m, 2H), 2.40-2.36 (m, 2H), 2.10 (s, 3H), 1.17 (t, J=7.2 Hz, 3H).

Step 2—Ethyl 3-(methyl (2-sulfamoylethyl)amino)propanoate. To a solution of ethyl 3-[2-[bis[(4-methoxyphenyl)methyl]sulfamoyl]ethyl-methyl-amino]propanoate (2.38 g, 4.97 mmol) in DCM (4 mL) was added TFA (6.16 g, 54.0 mmol, 4 mL). The mixture was stirred at 40° C., for 32 hrs. On completion, the reaction mixture was added Et₃N to adjust the pH to 8, and then was concentrated under reduced pressure to give a residue. The residue was purified by column chromatography (SiO₂, Petroleum ether/Ethyl acetate=2/1 to 0/1) to give the title compound (1.9 g, 96% yield) as a yellow solid. LC-MS (ESI⁺) m/z 239.0 (M+H)⁺; ¹H NMR (400 MHZ, DMSO-d₆) δ=6.99 (s, 2H), 4.08 (q, J=7.2 Hz, 2H), 3.23-2.87 (m, 7H), 2.64 (s, 2H), 1.18 (q, J=7.2 Hz, 6H).

### 3-((2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)(methyl)amino)propanoic acid (Intermediate GG)

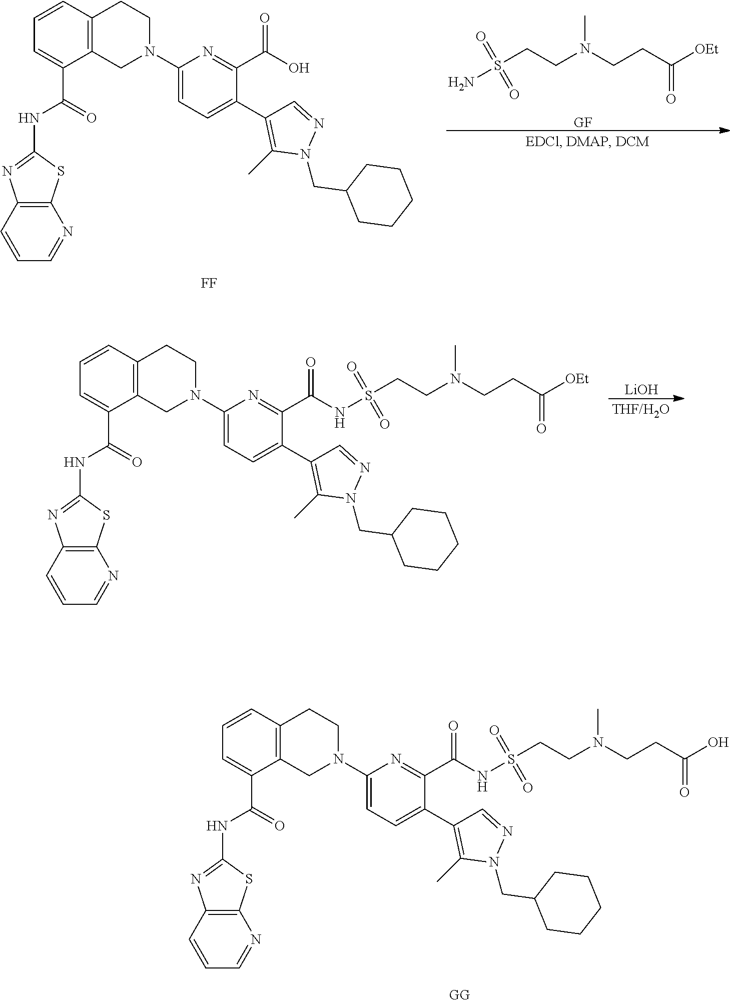

Step 1—Ethyl 3-((2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)(methyl)amino)propanoate. To a solution of 3-[1-(cyclohexylmethyl)-5-methyl-pyrazol-4-yl]-6-[8-(thiazolo[5,4-b]pyridin-2-ylcarbamoyl)-3,4-dihydro-1H-isoquinolin-2-yl]pyridine-2-carboxylic acid (319 mg, 525 umol, Intermediate FF) in DCM (5 mL) was added DMAP (256 mg, 2.10 mmol), EDCI (302 mg, 1.57 mmol) and ethyl 3-[methyl (2-sulfamoylethyl)amino]propanoate (150 mg, 629 umol, Intermediate GF). The mixture was then stirred at 20° C., for 12 hrs. On completion, the reaction mixture was washed with H₂O (5 mL×2), and the organic layer was concentrated under reduced pressure to give a residue. The residue was purified by column chromatography (SiO₂, DCM/MeOH=I/O to 10/1) to give the title compound (160 mg, 35% yield) as a yellow solid. LC-MS (ESI⁺) m/z 828.5 (M+H)⁺; ¹H NMR (400 MHZ, DMSO-d₆) δ=8.51 (dd, J=1.6, 4.8 Hz, 1H), 8.15 (dd, J=1.6, 8.0 Hz, 1H), 7.64 (d, J=7.6 Hz, 1H), 7.55-7.51 (m, 1H), 7.50 (d, J=8.4 Hz, 1H), 7.47-7.43 (m, 1H), 7.40-7.35 (m, 1H), 7.30 (s, 1H), 6.99-6.95 (m, 1H), 4.95 (s, 2H), 4.06-3.99 (m, 3H), 3.93 (t, J=6.0 Hz, 2H), 3.84 (d, J=7.2 Hz, 2H), 3.42 (s, 3H), 3.17 (s, 1H), 3.15-3.11 (m, 1H), 3.02 (t, J=5.6 Hz, 2H), 2.85-2.80 (m, 2H), 2.73-2.68 (m, 2H), 2.46-2.42 (m, 3H), 2.24 (s, 3H), 2.12 (s, 3H), 1.99 (s, 1H), 1.82-1.75 (m, 1H), 1.68-1.51 (m, 6H), 1.20-1.11 (m, 8H), 1.01-0.92 (m, 2H).

Step 2—3-((2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)(methyl)amino)propanoic acid. To a solution of ethyl 3-[2-[[3-[1-(cyclohexylmethyl)-5-methyl-pyrazol-4-yl]-6-[8-(thiazolo[5,4-b]pyridin-2-ylcarbamoyl)-3,4-dihydro-1H-isoquinolin-2-yl]pyridine-2-carbonyl]sulfamoyl]ethyl-methyl-amino]propanoate (150 mg, 181 umol) in THF (2 mL) and H₂O (2 mL) was added LiOH·H₂O (38.0 mg, 906 umol). The mixture was then stirred at 25° C., for 2 hrs. On completion, the reaction mixture was added 1 M HCl to adjust the pH to 6, and then extracted with EtOAc 20 mL (5 mL×4). The combined organic layers were concentrated under reduced pressure to give the title compound (142 mg) as a yellow solid. LC-MS (ESI⁺) m/z 800.3 (M+H)⁺. Benzyl 1-(2-sulfamoylethyl)azetidine-3-carboxylate (Intermediate GH)

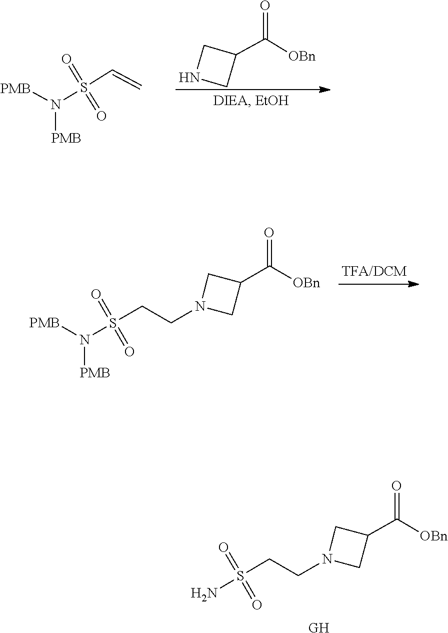

Step 1—5-benzyl 1-(2-(N,N-bis(4-methoxybenzyl)sulfamoyl)ethyl)azetidine-3-carboxylate. To a solution of N,N-bis[(4-methoxyphenyl)methyl]ethenesulfonamide (8 g, 23.0 mmol, Intermediate GA) and benzyl azetidine-3-carboxylate (18.0 g, 9.67 mmol, CAS #405513-07-7) in EtOH (10 mL) was added DIEA (11.9 g, 9.21 mmol). The mixture was then stirred at 60° C., for 2 hours. On completion, the reaction mixture was concentrated under reduced pressure to give a residue. The residue was purified by column chromatography (SiO₂, Petroleum ether/Ethyl acetate=1/1 to 0/1) to give a title compound (10.5 g, 81% yield) as a colorless oil. LC-MS (ESI⁺) m/z 539.4 (M+H)⁺.

Step 2—Benzyl 1-(2-sulfamoylethyl)azetidine-3-carboxylate. To a solution of benzyl 1-[2-[bis[(4-methoxyphenyl)methyl]sulfamoyl]ethyl]azetidine-3-carboxylate (10.5 g, 19.4 mmol) in DCM (60 mL) was added TFA (60 mL). The mixture was stirred at 40° C., for 24 hrs. On completion, the reaction mixture pH value of the solution to 7-8 by TEA, then diluted with H₂O (15 mL) and extracted with EA (40 mL×2). The combined organic layers were dried over Na₂SO₄, filtered and concentrated under reduced pressure to give a residue. The residue was purified by prep-HPLC (FA condition) to give a title compound (1 g, 16% yield) as a white solid. LC-MS (ESI⁺) m/z 299.1 (M+H)⁺.

### 1-(2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)azetidine-3-carboxylic acid (Intermediate GI)

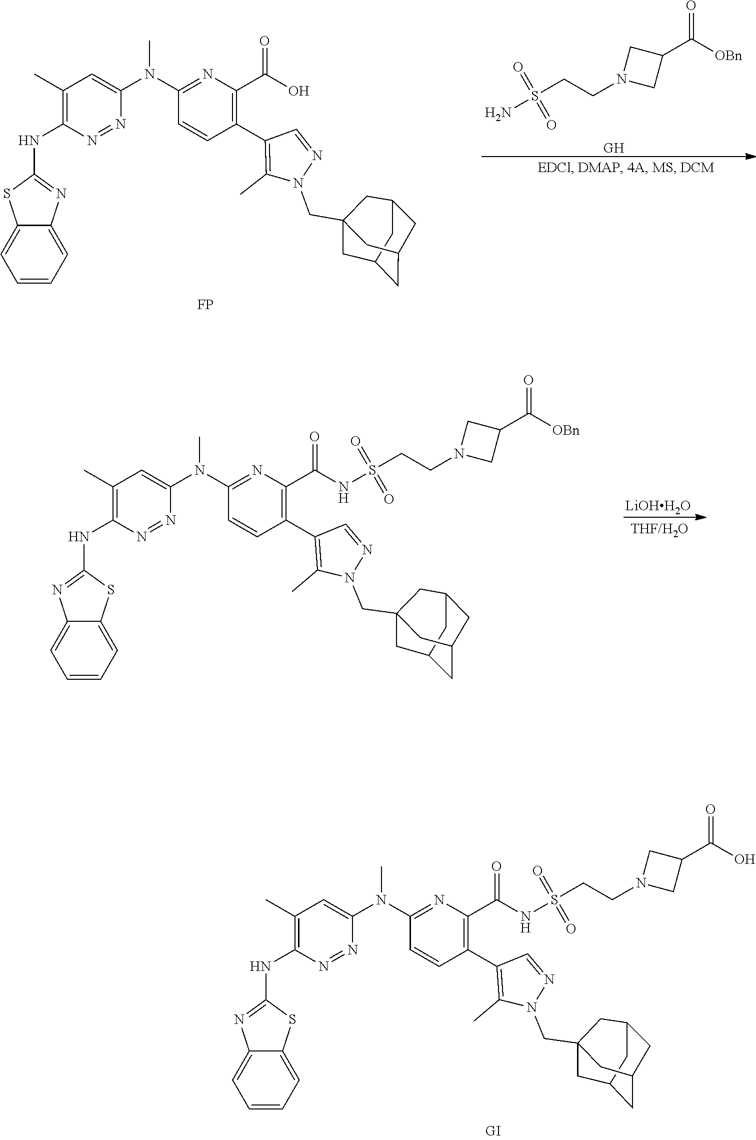

Step 1—Benzyl 1-(2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)azetidine-3-carboxylate. To a solution of 3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carboxylic acid (640 mg, 1.03 mmol, Intermediate FP) and benzyl 1-(2-sulfamoylethyl)azetidine-3-carboxylate (461 mg, 1.55 mmol, Intermediate GH) in DCM (10 mL) was added DMAP (503 mg, 4.12 mmol), EDCI (592 mg, 3.09 mmol) and 4 Å molecular sieves (150 mg). The mixture was stirred at 25° C., for 2 hr. On completion, the reaction mixture was diluted with H₂O (60 mL) and extracted with DCM (100 mL×3). The combined organic layers were dried over Na₂SO₄, filtered and concentrated under reduced pressure to give a residue. The residue was purified by column chromatography (SiO₂, Petroleum ether/Ethyl acetate=1/1 to 0/1, DCM:MeOH=10:1) to give the title compound (200 mg, 20% yield, 94% purity) as a white solid and impure title compound (600 mg, 30% yield, 47% purity) as a yellow solid. LC-MS (ESI⁺) m/z 901.5 (M+H)⁺.

Step 2—1-(2-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)ethyl)azetidine-3-carboxylic acid. To a solution of benzyl 1-[2-[[3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carbonyl]sulfamoyl]ethyl]azetidine-3-carboxylate (200 mg, 222 umol) in THF (2 mL) and H₂O (2 mL) was added LiOH·H₂O (55.8 mg, 1.33 mmol). The mixture was stirred at 25° C., for 2 hr. On completion, the reaction mixture pH value of the solution to 6-7 by NH₄Cl, then diluted with H₂O (25 mL) and extracted with DCM (30 mL×3). The combined organic layers were dried over Na₂SO₄, filtered and concentrated under reduced pressure to give a residue. The residue was purified by prep-HPLC (FA condition) to give a title compound (120 mg, 67% yield) as a yellow solid. LC-MS (ESI⁺) m/z 811.6 (M+H)⁺.

### Methyl 1-(4-sulfamoylphenyl) azetidine-3-carboxylate (Intermediate GJ)

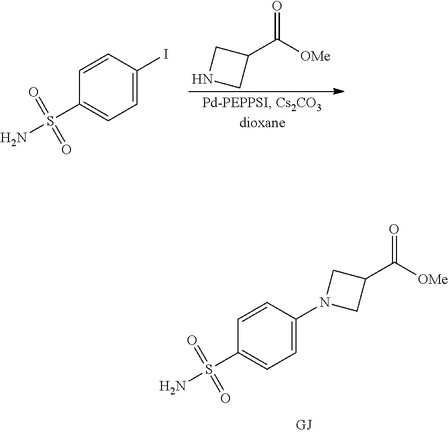 [SMILES: NS(=O)(=O)c1ccc(I)cc1 ; COC(=O)C1CNC1 ; COC(=O)C1CN(c2ccc(S(N)(=O)=O)cc2)C1]

A mixture of 4-iodobenzenesulfonamide (3.00 g, 10.6 mmol, CAS$825-86-5), methyl azetidine-3-carboxylate hydrochloride (3.21 g, 21.2 mmol, CAS #100202-39-9), Cs₂CO₃ (13.8 g, 42.4 mmol), and Pd-PEPPSI-IHeptCl (515 mg, 530 umol) in dioxane (130 mL) was degassed and purged with N₂ three times. Then the mixture was stirred at 100° C., for 3 hrs under N₂ atmosphere. On completion, the reaction mixture was filtered to give the filtrate and concentrated under reduced pressure. The mixture was purified by column chromatography (SiO₂, Petroleum ether/Ethyl acetate=I/O to 1/2) to give the title compound (2.40 g, 78% yield) as a yellow solid. LC-MS (ESI⁺) m/z 271.0 (M+H)⁺: ¹H NMR (400 MHZ, DMSO-d₆) δ=7.65-7.57 (m, 2H), 7.04 (s, 2H), 6.55-6.49 (m, 2H), 4.15-4.09 (m, 2H), 3.97 (dd, J=5.6, 7.6 Hz, 2H), 3.70-3.65 (m, 4H).

### Ethyl 1-(4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)phenyl) azetidine-3-carboxylic acid (Intermediate GK)

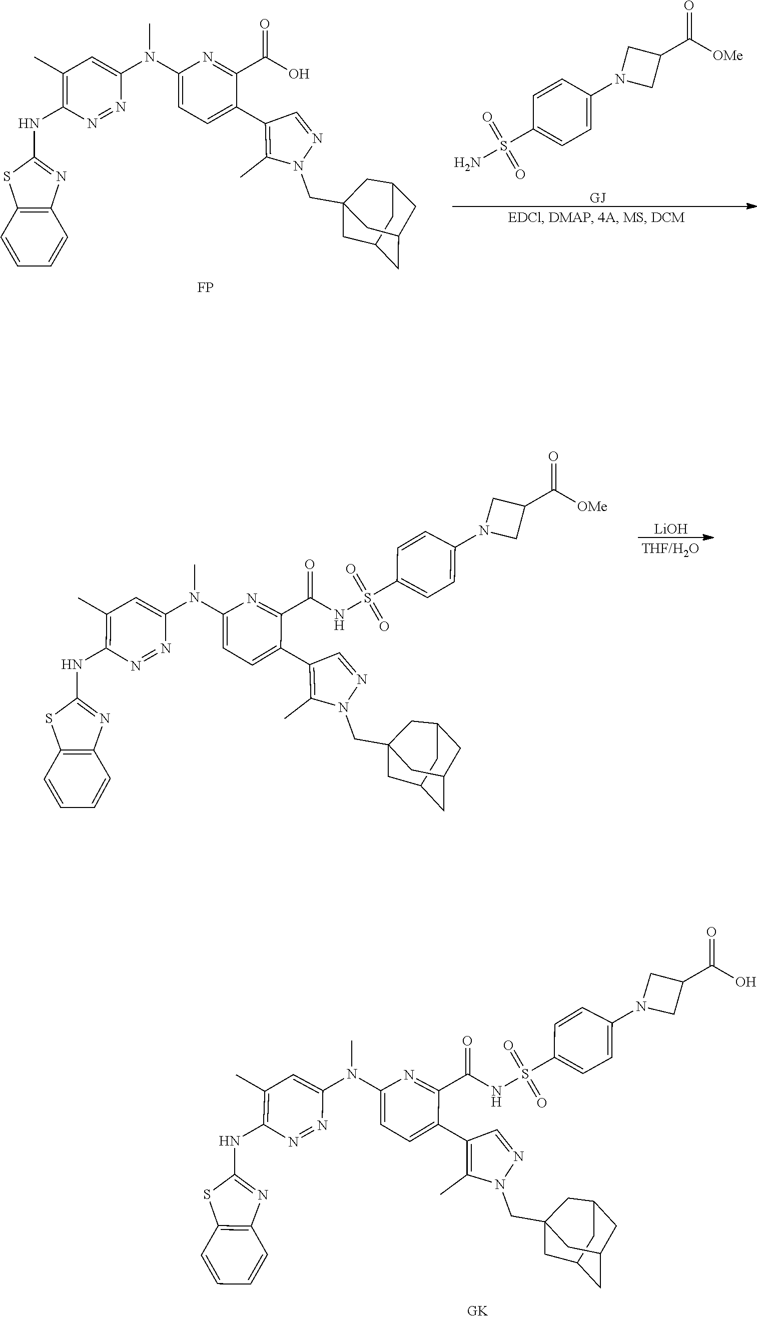

Step 1—Methyl 1-(4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)phenyl) azetidine-3-carboxylate. To a solution of 3-[1-(1-adamantylmethyl)-5-methyl-pyrazol-4-yl]-6-[6-(1,3-benzothiazol-2-ylamino)-5-methyl-pyridazin-3-yl]-methyl-amino]pyridine-2-carboxylic acid (500 mg, 805 umol, Intermediate FP) in DCM (10 mL) was added DMAP (394 mg, 3.22 mmol), EDCI (463 mg, 2.42 mmol), 4 Å molecular sieves (100 mg, 806 umol) and methyl 1-(4-sulfamoylphenyl) azetidine-3-carboxylate (283 mg, 1.05 mmol, Intermediate GJ). The mixture was stirred at 20° C., for 12 hrs. On completion, the reaction mixture was washed with H₂O (15 mL×2), and the organic layers were concentrated under reduced pressure to give a residue. The residue was purified by column chromatography (SiO₂, Petroleum ether/Ethyl acetate (1/1 to 0/1) to DCM:MeOH to THF) to give the title compound (530 mg, 68% yield) as a yellow solid. LC-MS (ESI⁺) m/z 873.9 (M+H)⁺. ¹H NMR (400 MHz, DMSO-d₆) δ=11.82-10.74 (m, 1H), 8.12 (d, J=1.6 Hz, 1H), 8.11 (d, J=1.6 Hz, 1H), 7.87 (d, J=8.0 Hz, 1H), 7.75 (d, J=0.8 Hz, 1H), 7.58-7.54 (m, 3H), 7.41-7.36 (m, 1H), 7.31 (s, 1H), 7.23-7.15 (m, 2H), 6.69 (d, J=1.6 Hz, 1H), 6.67 (d, J=1.6 Hz, 1H), 6.38 (d, J=8.8 Hz, 2H), 4.09-4.03 (m, 2H), 3.92 (dd, J=6.0, 7.2 Hz, 2H), 3.67-3.64 (m, 5H), 3.63 (d, J=3.2 Hz, 1H), 3.61 (s, 3H), 2.37 (s, 2H), 2.03 (s, 3H), 1.95 (s, 3H), 1.69-1.49 (m, 13H).

Step 2—Ethyl 1-(4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)phenyl) azetidine-3-carboxylic acid. To a solution of methyl 1-[4-[3-[1-(1-adamantylmethyl)-5-methyl-pyrazol-4-yl]-6-[[6-(1,3-benzothiazol-2-ylamino)-5-methyl-pyridazin-3-yl]-methyl-amino]pyridine-2-carbonyl]sulfamoyl]phenyl]azetidine-3-carboxylate (530 mg, 546 umol) in THF (4 mL) and H₂O (4 mL) was added LiOH·H₂O (115 mg, 2.73 mmol). The mixture was stirred at 20° C., for 2 hrs. On completion, to the reaction mixture was added 1 M HCl to adjust the pH to 6, and then extracted with DCM 30 mL (10 mL×3). The combined organic layers were concentrated under reduced pressure to give the title compound (520 mg) as a yellow solid. LC-MS (ESI⁺) m/z 859.3 (M+H)⁺. ¹H NMR (400 MHZ, DMSO-d₆) δ=8.11 (d, J=6.0 Hz, 2H), 7.87 (d, J=8.0 Hz, 1H), 7.76 (s, 1H), 7.56 (dd, J=4.4, 8.4 Hz, 3H), 7.41-7.36 (m, 1H), 7.31 (s, 1H), 7.23-7.15 (m, 2H), 6.68 (d, J=6.4 Hz, 2H), 6.38 (d, J=8.4 Hz, 2H), 5.75 (s, 1H), 4.08-4.01 (m, 2H), 3.96-3.90 (m, 2H), 3.65 (s, 2H), 3.62-3.60 (m, 3H), 3.54-3.49 (m, 1H), 2.36 (s, 2H), 2.01 (s, 3H), 1.94 (s, 3H), 1.70-1.48 (m, 13H).

### Methyl 1-(3-methyl-4-sulfamoylphenyl) azetidine-3-carboxylate (Intermediate GL)

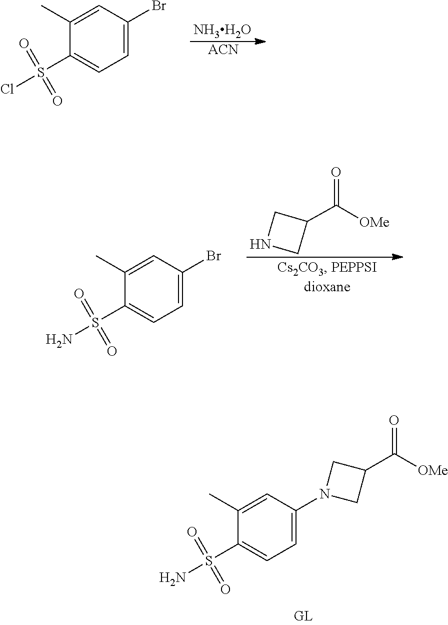 [SMILES: Cc1cc(Br)ccc1S(=O)(=O)Cl ; "ACN" ; "NH3·H2O" ; COC(=O)C1CNC1 ; Cc1cc(Br)ccc1S(N)(=O)=O ; COC(=O)C1CN(c2ccc(S(N)(=O)=O)c(C)c2)C1]

Step 1—4-bromo-2-methylbenzenesulfonamide. A solution of 4-bromo-2-methyl-benzenesulfonyl chloride (3 g, 11.1 mmol, CAS #139937-37-4) in ACN (30 mL) was added into NH₃·H₂O (16.6 g, 118 mmol, 25% solution). The mixture was then stirred at 0° C., for 1 hr. On completion, the reaction mixture was diluted with H₂O (100 mL) and extracted with EA (100 mL×3). The combined organic layers were dried over Na₂SO₄, filtered to give a filtrate and concentrated under reduced pressure to give the title compound (2.5 g) as a white solid. ¹H NMR (400 MHZ, DMSO-d₆) δ=7.75 (d, J=8.4 Hz, 1H), 7.63 (s, 1H), 7.58 (dd, J=1.7, 8.4 Hz, 1H), 7.39 (br s, 2H), 2.57 (s, 3H).

Step 2—Methyl 1-(3-methyl-4-sulfamoylphenyl) azetidine-3-carboxylate. A mixture of 4-bromo-2-methyl-benzenesulfonamide (360 mg, 1.44 mmol), methyl azetidine-3-carboxylate (436 mg, 2.88 mmol, HCl), 1,3-bis[2,6-bis(1-propylbutyl)phenyl]-4,5-dichloro-2H-imidazol-1-ium-2-ide: 3-chloropyridine dichloropalladium (70.0 mg, 71.9 umol), and Cs₂CO₃ (1.88 g, 5.76 mmol) in dioxane (2 mL) was degassed and purged with N₂ three times. Then the mixture was stirred at 100° C., for 2 hrs under N₂ atmosphere. On completion, the reaction mixture was filtered to get the filtrate and concentrated to give a residue. The residue was purified by column chromatography (SiO₂, Petroleum ether/Ethyl acetate=I/O to 1/1) to give the title compound (240 mg, 58% yield) as a yellow solid. LC-MS (ESI⁺) m/z 285.3 (M+H)⁺.

### 1-(4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)-3-methylphenyl) azetidine-3-carboxylic acid (Intermediate GM)

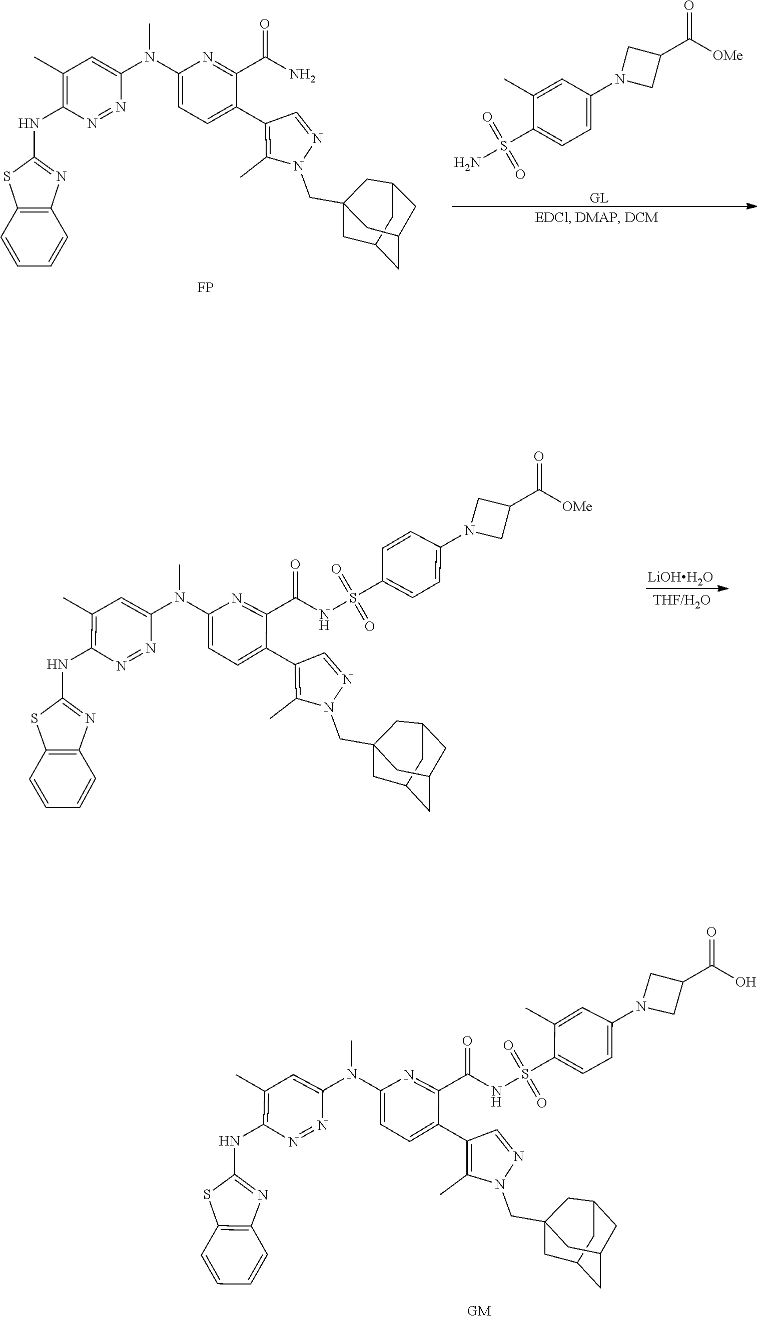

Step 1—Methyl 1-(4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)-3-methylphenyl) azetidine-3-carboxylate. To a solution of 3-[1-(1-adamantylmethyl)-5-methyl-pyrazol-4-yl]-6-[6-(1,3-benzothiazol-2-ylamino)-5-methyl-pyridazin-3-yl]-methyl-amino]pyridine-2-carboxylic acid (470 mg, 757 umol, Intermediate FP) and methyl 1-(3-methyl-4-sulfamoyl-phenyl) azetidine-3-carboxylate (215 mg, 757 umol, Intermediate GL) in DCM (10 mL) was added DMAP (370 mg, 3.03 mmol), EDCI (290 mg, 1.51 mmol) and 4A molecular sieves (50 mg, 757 umol). The mixture was stirred at 25° C., for 1 hr. On completion, the reaction mixture was filtered to give a filtrate and concentrated under reduced pressure to give a residue. The residue was purified by column chromatography (SiO₂, Petroleum ether/Ethyl acetate=I/O to 0/1) to give the title compound (500 mg, 71% yield) as a white solid. LC-MS (ESI⁺) m/z 887.3 (M+H)⁺.

Step 2—1-(4-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)-3-methylphenyl) azetidine-3-carboxylic acid. To a solution of methyl 1-[4-[3-[1-(1-adamantylmethyl)-5-methyl-pyrazol-4-yl]-6-[6-(1,3-benzothiazol-2-ylamino)-5-methyl-pyridazin-3-yl]-methyl-amino]pyridine-2-carbonyl]sulfamoyl]-3-methyl-phenyl]azetidine-3-carboxylate (500 mg, 535 umol) in THF (2 mL) and H₂O (2 mL) was added LiOH·H₂O (112.35 mg, 2.68 mmol). The mixture was then stirred at 25° C., for 1 hr. On completion, the reaction mixture pH value of the solution was adjusted to 6-7 with NH₄Cl, then diluted with H₂O (25 mL) and extracted with EA (30 mL×3). The combined organic layers were dried over Na₂SO₄, filtered to give a filtrate and concentrated under reduced pressure to give a residue. The residue was purified by prep-HPLC (FA condition) to give a title compound (350 mg, 71% yield) as a yellow solid. LC-MS (ESI⁺) m/z 873.4 (M+H)⁺.

### N-(6-chloro-4-methylpyridazin-3-yl)-N-((2-(trimethylsilyl)ethoxy)methyl) thiazolo[5,4-b]pyridin-2-amine (Intermediate GN)

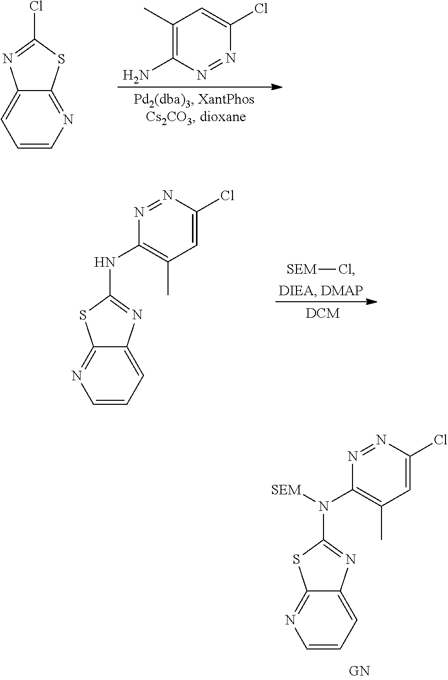

Step 1—N-(6-chloro-4-methylpyridazin-3-yl) thiazolo[5,4-b]pyridin-2-amine. To a solution of 2-chlorothiazolo[5,4-b]pyridine (4.50 g, 26.4 mmol, CAS #91524-96-8), 6-chloro-4-methyl-pyridazin-3-amine (4.54 g, 31.7 mmol, CAS #64068-00-4) and Cs₂CO; (25.8 g, 79.1 mmol) in dioxane (90 mL) was added Pd₂(dba); (1.21 g, 1.32 mmol) and Xantphos (1.53 g, 2.64 mmol) under N₂. The mixture was then stirred at 60° C., for 12 h. On completion, the reaction mixture was diluted with water (100 mL), EtOAc (100 mL) and PE (100 mL). The mixture was filtered and the filtered cake was dried to afford the title compound (7.50 g, 82% yield) as a brown solid. LC-MS (ESI⁺) m/z 278.0 (M+H)⁺.

Step 2 N-(6-chloro-4-methylpyridazin-3-yl)-N-((2-(trimethylsilyl)ethoxy)methyl) thiazolo[5,4-b]pyridin-2-amine. To a solution of N-(6-chloro-4-methylpyridazin-3-yl) thiazolo[5,4-b]pyridin-2-amine (7.50 g, 21.6 mmol, 80% purity), DIEA (8.38 g, 64.8 mmol, 11.3 mL) and DMAP (132 mg, 1.08 mmol) in DCM (150 mL) was added SEM-CI (3.96 g, 23.8 mmol, 4.21 mL) at 0° C., under N₂. The mixture was stirred at 0° C., for 1 h. On completion, the reaction mixture was diluted with water (100 mL) and extracted with DCM (100 mL×3). The combined organic layers were washed with brine (40 mL×3), dried over Na₂SO₄ and evaporated. The residue was purified by column chromatography (SiO₂, DCM/EtOAc=0:1 to 10:1) to afford the title compound (6.00 g, 68% yield) as a yellow solid. LC-MS (ESI⁺) m/z 408.1 (M+H)⁺.

### 3-(1-(cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinic acid (Intermediate GO)

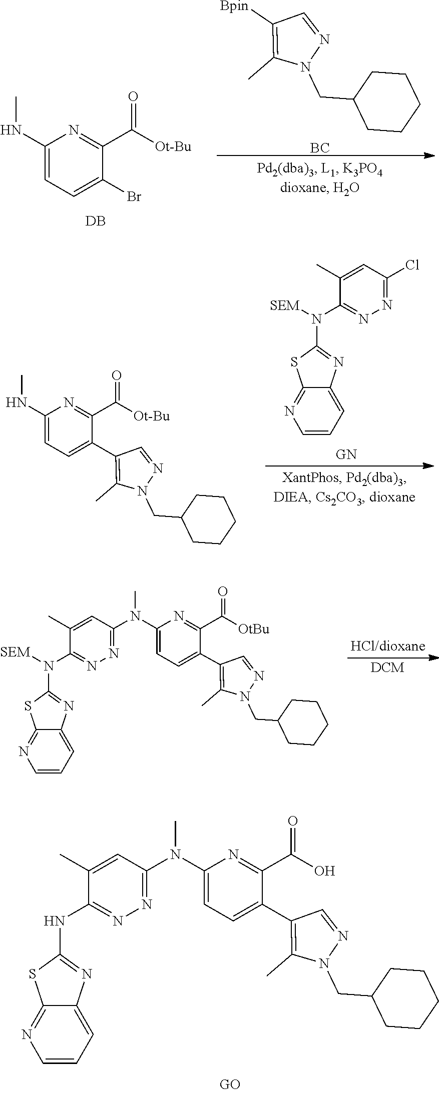

Step 1—Tert-butyl 3-(1-(cyclohexylmethyl)-5-methyl-1H-pyrazol-4-yl)-6-(methylamino)picolinate. To a solution of tert-butyl 3-bromo-6-(methylamino)picolinate (10.0 g, 34.8 mmol, Intermediate DB) and 1-(cyclohexylmethyl)-5-methyl-4-(4,4,5,5-tetramethyl-1,3,2-dioxaborolan-2-yl)-1H-pyrazole (10.6 g, 34.8 mmol, Intermediate BC) in dioxane (70 mL) and H₂O (70 mL) was added Pd₂(dba); (797 mg, 871 umol), (1S,3R,5R,7S)-1,3,5,7-tetramethyl-8-phenyl-2,4,6-trioxa-8-phosphatricyclo[3.3.1.1^{3,7}]decane (4.07 g, 13.9 mmol, L1) and K₃PO₄ (22.2 g, 104 mmol). The mixture was then stirred at 100° C., for 4 h under N₂. On completion, the reaction mixture was poured into water (70 mL), and then extracted with EtOAc (70 mL×3). The combined organic layers were washed with brine (70 mL×3), dried over Na₂SO₄, filtered and concentrated under reduced pressure to give a residue. The residue was purified by column chromatography (SiO₂, Dichloromethane:Methanol=20/1-10/1) to give the title compound (13.0 g, 97% yield) as a yellow solid. ¹H NMR (400 MHZ, DMSO-d₆) δ=7.27 (d, J=8.8 Hz, 1H), 7.19 (s, 1H), 6.70 (q, J=4.8 Hz, 1H), 6.53 (d, J=8.8 Hz, 1H), 3.86 (d, J=7.6 Hz, 2H), 2.76 (d, J=5.2 Hz, 3H), 2.08 (s, 3H), 1.81-1.72 (m, 1H), 1.66 (br d, J=5.6 Hz, 2H), 1.62-1.53 (m, 3H), 1.27 (s, 9H), 1.17-1.11 (m, 3H), 1.03-0.93 (m, 2H).

Step 2—Tert-butyl 3-(1-(cyclohexylmethyl)-5-methyl-1H-pyrazol-4-yl)-6-(methyl (5-methyl-6-(thiazolo[5,4-b]pyridin-2-yl((2-(trimethylsilyl)ethoxy)methyl)amino)pyridazin-3-yl)amino)picolinate. To a solution of tert-butyl 3-(1-(cyclohexylmethyl)-5-methyl-1H-pyrazol-4-yl)-6-(methylamino)picolinate (4.00 g, 10.4 mmol), N-(6-chloro-4-methylpyridazin-3-yl)-N-((2-(trimethylsilyl)ethoxy)methyl) thiazolo[5,4-b]pyridin-2-amine (5.09 g, 12.5 mmol, Intermediate GN), and Cs₂CO₃ (10.2 g, 31.21 mmol) in dioxane (50 mL) was added (5-diphenylphosphanyl-9,9-dimethyl-xanthen-4-yl)-diphenyl-phosphane (241 mg, 416 umol), (1E,4E)-1,5-diphenylpenta-1,4-dien-3-one palladium (953 mg, 1.04 mmol), and DIEA (4.03 g, 31.2 mmol, 5.44 mL) under N₂. The mixture was then stirred at 120° C. for 12 h. On completion, the reaction mixture was quenched with water (20 mL), and then extracted with EtOAc (10 mL×3). The combined organic layers were washed with brine (10 mL×3), dried over Na₂SO₄, filtered and concentrated under reduced pressure to give a residue. The residue was purified by column chromatography (SiO₂, Dichloromethane:Methanol=20/1-10/1) to give the title compound (3.30 g, 34% yield) as a yellow oil. LC-MS (ESI⁺) m/z 756.9 (M+H)⁺.

Step 3—3-(1-(Cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinic acid. Tert-butyl 3-(1-(cyclohexylmethyl)-5-methyl-1H-pyrazol-4-yl)-6-(methyl (5-methyl-6-(thiazolo[5,4-b]pyridin-2-yl((2-(trimethylsilyl)ethoxy)methyl)amino)pyridazin-3-yl)amino)picolinate (2.30 g, 3.04 mmol) in HCl/dioxane (15 mL) and DCM (10 mL) was stirred at 25° C., for 12 h. On completion, the solution was concentrated and freeze-drying in vacuo to give the title compound (1.97 g) as an orange solid. LC-MS (ESI⁺) m z 570.1 (M+H)⁺.

### 6-(N-(3-(1-(cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoic acid (Intermediate GP)

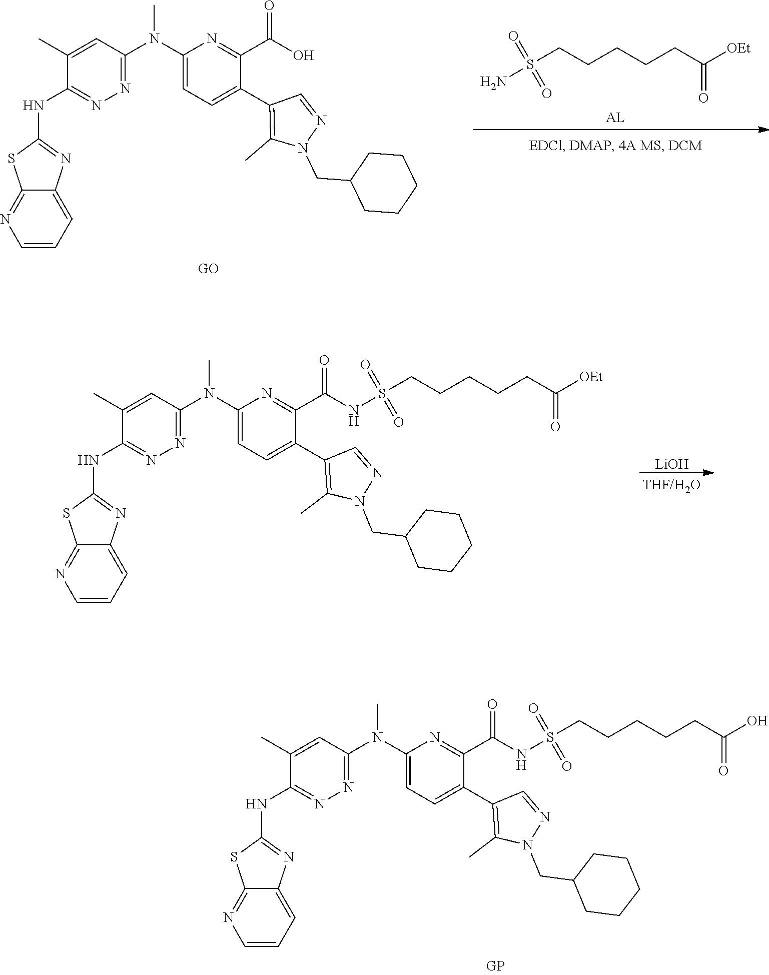 [SMILES: Cc1cc(N(C)c2ccc(-c3cnn(CC4CCCCC4)c3C)c(C(=O)O)n2)nnc1Nc1nc2cccnc2s1 ; CCOC(=O)CCCCCS(N)(=O)=O ; "AL" ; CCOC(=O)CCCCCS(=O)(=O)NC(=O)c1nc(N(C)c2cc(C)c(Nc3nc4cccnc4s3)nn2)ccc1-c1cnn(CC2CCCCC2)c1C ; "LiOH" ; Cc1cc(N(C)c2ccc(-c3cnn(CC4CCCCC4)c3C)c(C(=O)NS(=O)(=O)CCCCCC(=O)O)n2)nnc1Nc1nc2cccnc2s1]

Step 1—Ethyl 6-(N-(3-(1-(cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoate. To a solution of 3-(1-(cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinic acid (400 mg, 702 umol, Intermediate GO), ethyl 6-sulfamoylhexanoate (470 mg, 2.11 mmol, Intermediate AL) in DCM (5 mL) was added EDCI (269 mg, 1.40 mmol), DMAP (343 mg, 2.81 mmol), and 4 Å molecular sieves (0.50 g, 702 umol). Then the mixture was stirred at 20° C., for 13 h. On completion, the reaction mixture was quenched was H₂O (10 mL) and extracted with DCM (15 mL×3). The combined organic layers were washed with brine (4 mL×3), dried over Na₂SO₄ and evaporated. The crude product was purified by reversed-phase HPLC (0.1% FA condition, 5%-62%, 40 min). The title compound (153 mg, 28% yield) as a yellow solid. LC-MS (ESI⁺) m/z 775.7 (M+H)⁺.

Step 2—6-(N-(3-(1-(cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoic acid. A solution of ethyl 6-(N-(3-(1-(cyclohexylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoate (153 mg, 197 umol) and LiOH·H₂O (41.4 mg, 987 umol) in THF (3 mL) and H₂O (2 mL) was stirred at 25° C., for 3 h. On completion, the reaction mixture was filtered and extracted with DCM (5 mL×2). The aqueous phase pH of the aqueous phase was adjusted to 4˜5 with 2M HCl and extracted with DCM (5 mL×8). The combined organic layers were dried over Na₂SO₄ and evaporated to give the title compound (180 mg) as an orange solid. LC-MS (ESI⁺) m/z 747.6 (M+H)⁺.

### 6-((6-(Benzo[d]thiazol-2-ylamino)-5-methylpyridazin-3-yl)(methyl)amino)-3-(1-(cyclopentylmethyl)-5-methyl-1H-pyrazol-4-yl)picolinic acid (Intermediate GQ)

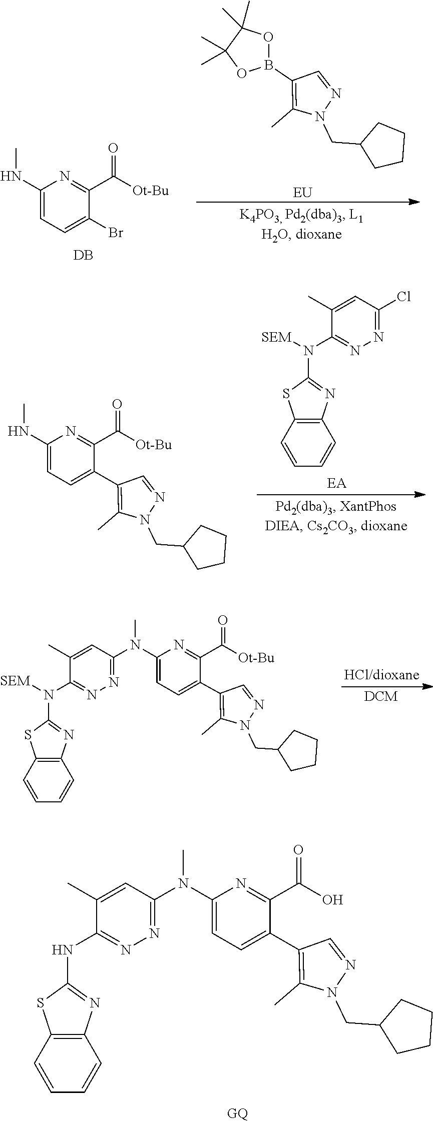

Step 1—Tert-butyl 3-(1-(cyclopentylmethyl)-5-methyl-1H-pyrazol-4-yl)-6-(methylamino)picolinate. To a solution of tert-butyl 3-bromo-6-(methylamino)picolinate (1.00 g, 3.48 mmol, Intermediate DB) and 1-(cyclopentylmethyl)-5-methyl-4-(4,4,5,5-tetramethyl-1,3,2-dioxaborolan-2-yl)-1H-pyrazole (1.31 g, 4.53 mmol, Intermediate EU) in H₂O (5 mL) and dioxane (5 mL) was added (1S,3R,5R,7S)-1,3,5,7-tetramethyl-8-phenyl-2,4,6-trioxa-8-phosphatricyclo[3.3.1.1^{3,7}]decane (204 mg, 696 umol, LI), K₃PO₄ (2.22 g, 10.5 mmol), and Pd₂(dba); (159 mg, 174 umol) at 20° C. Then the reaction was stirred from 20° C., to 100° C., for 12 h. On completion, the mixture was quenched with H₂O (10 mL) and extracted with DCM (10 mL×3). The combined organic layers was washed with brine (10 mL×3) and dried over Na₂SO₄ and concentrated in vacuo. The crude product was purified by reversed-phase HPLC (0.1% FA condition) to give the title compound (1.13 g, 77% yield) as a yellow solid. LC-MS (ESI⁺) m/z 371.3 (M+1)⁺.

Step 2—Tert-butyl 6-((6-(benzo[d]thiazol-2-yl((2-(trimethylsilyl)ethoxy)methyl)amino)-5-methylpyridazin-3-yl)(methyl)amino)-3-(1-(cyclopentylmethyl)-5-methyl-1H-pyrazol-4-yl)picolinate. To a solution of tert-butyl 3-[1-(cyclopentylmethyl)-5-methyl-pyrazol-4-yl]-6-(methylamino)pyridine-2-carboxylate (770 mg, 2.08 mmol) and N-(6-chloro-4-methyl-pyridazin-3-yl)-N-(2-trimethylsilylethoxymethyl)-1,3-benzothiazol-2-amine (1.27 g, 3.12 mmol, Intermediate EA) in dioxane (15 mL) was added Pd₂(dba); (190 mg, 208.0 umol), Xantphos (241.0 mg, 416.0 umol), DIEA (807 mg, 6.24 mmol), and Cs₂CO₃ (2.03 g, 6.24 mmol) at 25° C. Then the reaction was stirred at 120° C., for 12 h. On completion, the mixture was quenched with H₂O (15 mL) and extracted with DCM (15 mL×3). The combined organic layers was washed with brine (15 mL×3), dried over Na₂SO₄ and concentrated in vacuo. The residue was purified by column chromatography on silica gel (eluted with petroleum ether:ethyl acetate=5:1 to 3:1) to give the title compound (918 mg, 51% yield) as a yellow solid. LC-MS (ESI⁺) m/z 742.5 (M+1)⁺.

Step 3—6-((6-(Benzo[d]thiazol-2-ylamino)-5-methylpyridazin-3-yl)(methyl)amino)-3-(1-(cyclopentylmethyl)-5-methyl-1H-pyrazol-4-yl)picolinic acid. To a solution of tert-butyl 6-((6-(benzo[d]thiazol-2-yl((2-(trimethylsilyl)ethoxy)methyl)amino)-5-methylpyridazin-3-yl)(methyl)amino)-3-(1-(cyclopentylmethyl)-5-methyl-1H-pyrazol-4-yl)picolinate (560 mg, 756 umol) in DCM (5.20 mL) was added HCl/dioxane (4.00 M, 5.20 mL) at 25° C. Then the reaction was stirred at 25° C., for 4 h. On completion, the reaction was concentrated to give the title compound (400 mg, 88% yield) as a yellow solid. LC-MS (ESI⁺) m/z 555.4 (M+1)⁺.

### 1-(4-(N-(6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinoyl)sulfamoyl)phenyl)piperidine-4-carboxylic acid (Intermediate GR)

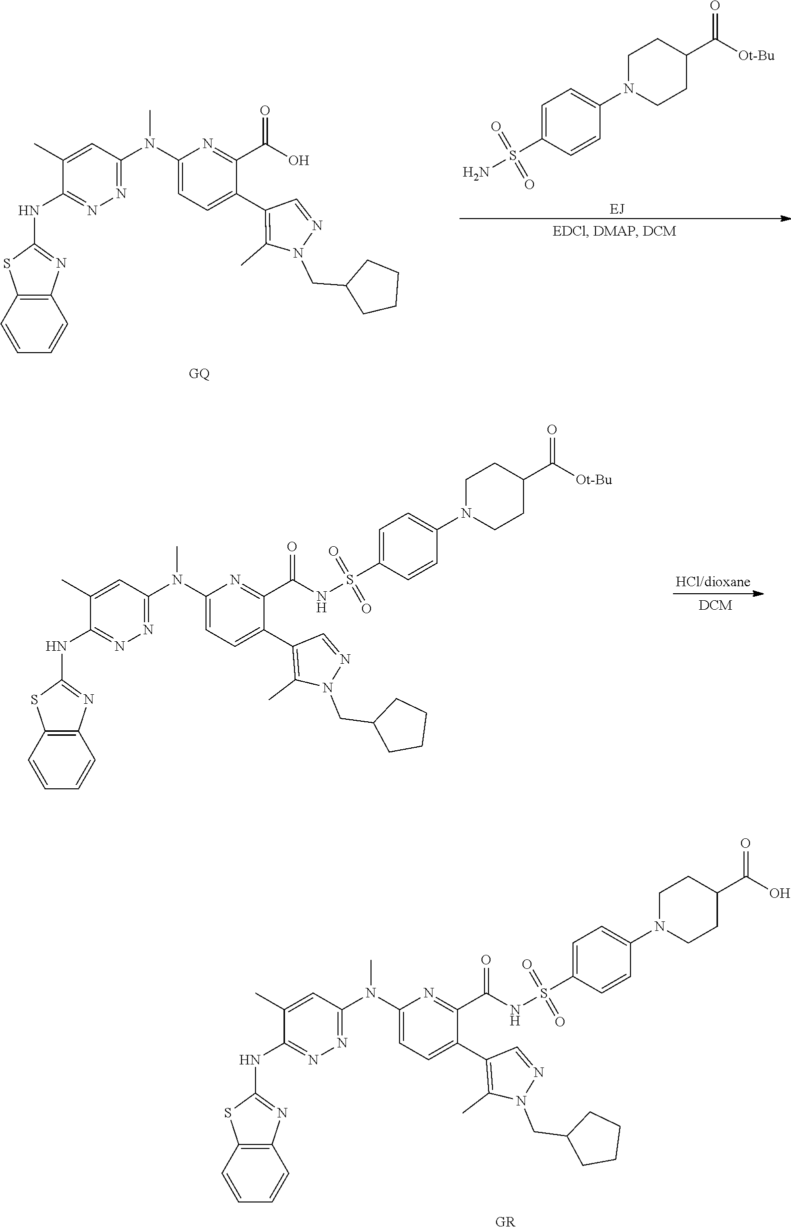 [SMILES: Cc1cc(N(C)c2ccc(-c3cnn(CC4CCCC4)c3C)c(C(=O)O)n2)nnc1Nc1nc2ccccc2s1 ; CC(C)(C)OC(=O)C1CCN(c2ccc(S(N)(=O)=O)cc2)CC1 ; "EJ" ; Cc1cc(N(C)c2ccc(-c3cnn(CC4CCCC4)c3C)c(C(=O)NS(=O)(=O)c3ccc(N4CCC(C(=O)OC(C)(C)C)CC4)cc3)n2)nnc1Nc1nc2ccccc2s1 ; Cc1cc(N(C)c2ccc(-c3cnn(CC4CCCC4)c3C)c(C(=O)NS(=O)(=O)c3ccc(N4CCC(C(=O)O)CC4)cc3)n2)nnc1Nc1nc2ccccc2s1]

Step 1—Tert-butyl 1-(4-(N-(6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinoyl)sulfamoyl)phenyl)piperidine-4-carboxylate. To a solution of 6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinic acid (290 mg, 523 umol, Intermediate GQ) and tert-butyl 1-(4-sulfamoylphenyl)piperidine-4-carboxylate (267.0 mg, 784.0 umol, Intermediate EJ) in DCM (3.50 mL) was added EDCI (200 mg, 1.05 mmol), DMAP (256.0 mg, 2.09 mmol) and 4 Å molecular sieves (15.0 mg) at 25° C. Then the reaction was stirred at 25° C., for 2h. On completion, the mixture was quenched with H₂O (4 mL) and extracted with DCM (4 mL×3). The combined organic layers was washed with brine (4 mL×3), dried over Na₂SO₄ and concentrated in vacuo. The crude product was purified by reversed-phase HPLC (0.1% FA condition) to give the title compound (220 mg, 47% yield) as a yellow solid. LC-MS (ESI⁺) m/z 877.5 (M+1)⁺. ¹H NMR (400 MHZ, DMSO-d₆) δ 12.06-11.62 (m, 1H), 7.89 (br d, J=6.8 Hz, 1H), 7.82 (s, 1H), 7.67-7.49 (m, 4H), 7.39 (t, J=7.6 Hz, 1H), 7.30 (br d, J=8.4 Hz, 1H), 7.26-7.16 (m, 2H), 6.94 (br d, J=9.2 Hz, 2H), 3.87 (d, J=7.6 Hz, 2H), 3.76 (br d, J=12.4 Hz, 2H), 3.63 (s, 3H), 2.95-2.84 (m, 2H), 2.43 (br s, 3H), 2.33-2.26 (m, 1H), 1.91 (s, 3H), 1.79-1.71 (m, 2H), 1.65-1.54 (m, 5H), 1.53-1.47 (m, 3H), 1.39 (s, 9H), 1.27-1.20 (m, 2H).

Step 2—1-(4-(N-(6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinoyl)sulfamoyl)phenyl)piperidine-4-carboxylic acid. To a solution of tert-butyl 1-(4-(N-(6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinoyl)sulfamoyl)phenyl)piperidine-4-carboxylate (220 mg, 251 umol) in DCM (2.50 mL) was added HCl/dioxane (4 M, 62.7 uL) at 25° C. Then the reaction was stirred at 25° C., for 12 h. On completion, the reaction was concentrated to give the title compound (240 mg, 98% yield) as a yellow solid. LC-MS (ESI⁺) m/z 821.5 (M+1)⁺.

### 6-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)hexanoic acid (Intermediate GS)

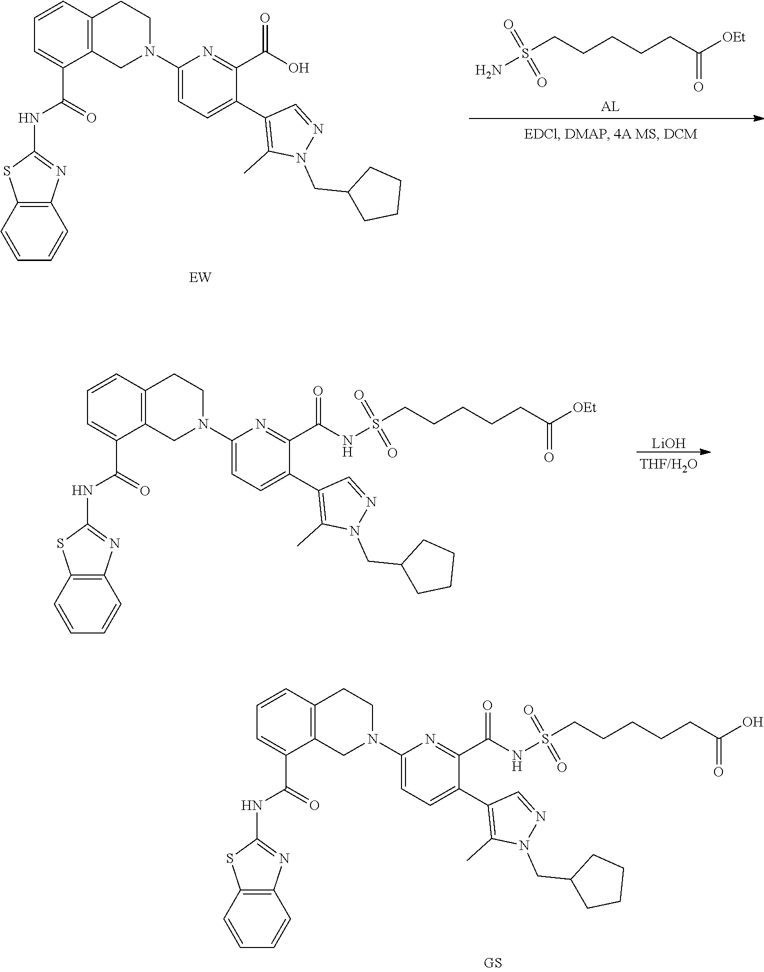 [SMILES: Cc1c(-c2ccc(N3CCc4cccc(C(=O)Nc5nc6ccccc6s5)c4C3)nc2C(=O)O)cnn1CC1CCCC1 ; CCOC(=O)CCCCCS(N)(=O)=O ; "AL" ; CCOC(=O)CCCCCS(=O)(=O)NC(=O)c1nc(N2CCc3cccc(C(=O)Nc4nc5ccccc5s4)c3C2)ccc1-c1cnn(CC2CCCC2)c1C ; "LiOH" ; Cc1c(-c2ccc(N3CCc4cccc(C(=O)Nc5nc6ccccc6s5)c4C3)nc2C(=O)NS(=O)(=O)CCCCCC(=O)O)cnn1CC1CCCC1]

Step 1—Ethyl 6-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)hexanoate. To a solution of 6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinic acid (800 mg, 1.35 mmol, Intermediate EW) and ethyl 6-sulfamoylhexanoate (1.05 g, 4.72 mmol, Intermediate AL), 4 Å molecular sieves (1 g) in DCM (8 mL) was added EDCI (517 mg, 2.70 mmol) and DMAP (660 mg, 5.40 mmol). The mixture was stirred at 25° C., for 8 h. On completion, the reaction mixture was quenched with H₂O (5 mL), and extracted with DCM (5 mL×3). The organic layers were washed with brine (5 mL×3), dried over Na₂SO₄, filtered and concentrated under reduced pressure to give a residue. The crude product was purified by reversed-phase HPLC (0.1% FA condition) to give the title compound (450 mg, 42% yield) as brown gum. LC-MS (ESI⁺) m/z 798.1 (M+H)⁺.

Step 2—6-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)hexanoic acid. To a solution of ethyl 6-(N-(6-(8-(benzo[d]thiazol-2-ylcarbamoyl)-3,4-dihydroisoquinolin-2 (1H)-yl)-3-(1-(cyclopentylmethyl)-5-methyl-1H-pyrazol-4-yl)picolinoyl)sulfamoyl)hexanoate (450 mg, 564 umol) in H₂O (0.9 mL) and THF (3.6 mL) was added LiOH·H₂O (94.7 mg, 2.26 mmol). The mixture was stirred at 40° C., for 3 h. On completion, the reaction mixture was filtered and diluted with H₂O (2 mL), adjusted to pH=3˜4 with IM HCl, then the mixture was extracted with DCM (5 mL×3). The combined organic layer was washed with brine (3 mL×3), dried over Na₂SO₄, filtered and concentrated in vacuo to give the title compound (290 mg) as yellow gum. LC-MS (ESI⁺) m/z 770.5 (M+H)⁺.

### 6-[[6-[[6-(1,3-Benzothiazol-2-ylamino)-5-methyl-pyridazin-3-yl]-methyl-amino]-3-[1-(cyclopentylmethyl)-5-methyl-pyrazol-4-yl]pyridine-2-carbonyl]sulfamoyl]hexanoic acid (Intermediate GT)

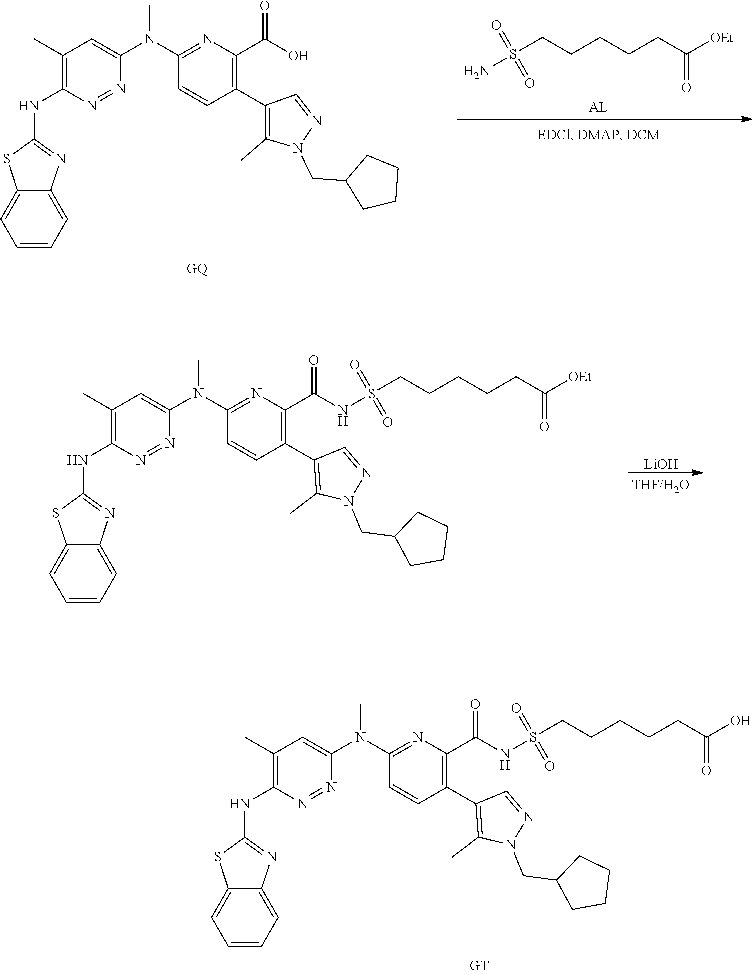 [SMILES: Cc1cc(N(C)c2ccc(-c3cnn(CC4CCCC4)c3C)c(C(=O)O)n2)nnc1Nc1nc2ccccc2s1 ; CCOC(=O)CCCCCS(N)(=O)=O ; "AL" ; CCOC(=O)CCCCCS(=O)(=O)NC(=O)c1nc(N(C)c2cc(C)c(Nc3nc4ccccc4s3)nn2)ccc1-c1cnn(CC2CCCC2)c1C ; "LiOH" ; Cc1cc(N(C)c2ccc(-c3cnn(CC4CCCC4)c3C)c(C(=O)NS(=O)(=O)CCCCCC(=O)O)n2)nnc1Nc1nc2ccccc2s1]

Step 1—Ethyl 6-(N-(6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinoyl)sulfamoyl)hexanoate. To a solution of 6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinic acid (186 mg, 335 umol, Intermediate GQ) and ethyl 6-sulfamoylhexanoate (225 mg, 1.01 mmol, Intermediate AL) in DCM (2 mL) was added EDCI (257.0 mg, 1.34 mmol) and DMAP (328.0 mg, 2.68 mmol) at 20° C. Then the reaction was stirred at 40° C. for 12 h. On completion, the mixture was quenched with H₂O (2 mL) and extracted with DCM (2 mL×3). The combined organic layers was washed with brine (1.5 mL×3), dried over Na₂SO₄ and concentrated in vacuo. The crude product was purified by reversed-phase HPLC (0.1% FA condition, 5%-70%) to give the title compound (76.0 mg, 29% yield) as a yellow solid. LC-MS (ESI⁺) m/z 760.3 (M+1)⁺.

Step 2—6-[[6-[[6-(1,3-Benzothiazol-2-ylamino)-5-methyl-pyridazin-3-yl]-methyl-amino]-3-[1-(cyclopentylmethyl)-5-methyl-pyrazol-4-yl]pyridine-2-carbonyl]sulfamoyl]hexanoic acid. To a solution of 6-(N-(6-((6-(benzo[d]thiazol-2-ylamino)-5-methylpyridazin-3-yl)(methyl)amino)-3-(1-(cyclopentylmethyl)-5-methyl-1H-pyrazol-4-yl)picolinoyl)sulfamoyl)hexanoate (76.0 mg, 100 umol) in THF (1.5 mL) and H₂O (1.5 mL) was added LiOH·H₂O (21.0 mg, 500 umol) at 20° C. Then the reaction was stirred at 20° C., for 1 h. On completion, the reaction was poured into water (3 mL) and acidized by 2M HCl to pH=4, and extracted with ethyl acetate (5 mL×2). The combined organic phase was washed with brine (3 mL×2), dried over Na₂SO₄ and concentrated in vacuo to give the title compound (65.0 mg, 82% yield) as a yellow solid. LC-MS (ESI⁺) m/z 732.1 (M+1)⁺.

### 3-(1-(Cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinic acid (Intermediate GU)

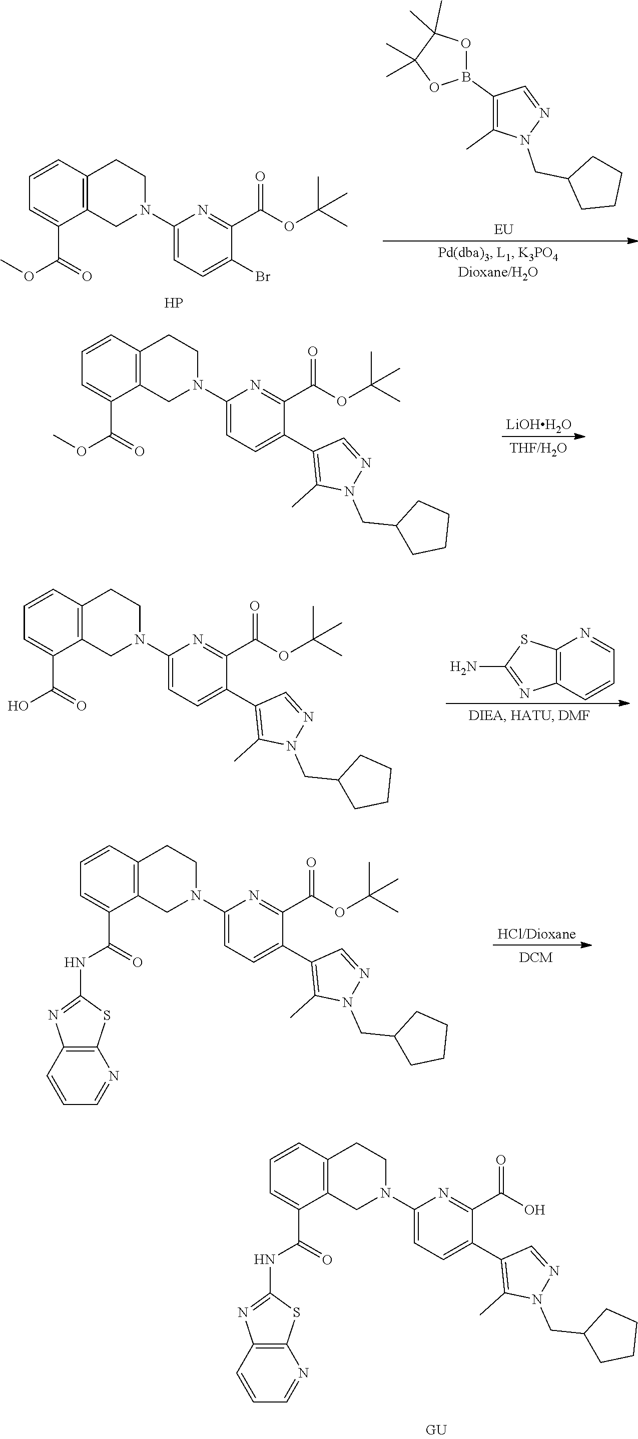

Step 1—Methyl-2-(6-(tert-butoxycarbonyl)-5-(1-(cyclopentylmethyl)-5-methyl-1H-pyrazol-4-yl) pyridin-2-yl)-1,2,3,4-tetrahydroisoquinoline-8-carboxylate. A mixture of methyl 2-(5-bromo-6-tert-butoxycarbonyl-2-pyridyl)-3,4-dihydro-1H-isoquinoline-8-carboxylate (2.06 g, 4.59 mmol. Intermediate HP). 1-(cyclopentylmethyl)-5-methyl-4-(4,4,5,5-tetramethyl-1,3,2-dioxaborolan-2-yl)-1H-pyrazole (2 g. 6.89 mmol. Intermediate EU). Pd₂(dba); (336 mg. 367 umol). (1S,3R,5R,7S)-1,3,5,7-tetramethyl-8-phenyl-2,4,6-trioxa-8-phosphatricyclo[3.3.1.1^{3,7}]decane (470 mg, 1.61 mmol. L1) and K₃PO₄ (2.93 g, 13.7 mmol) in dioxane (15 mL) and H₂O (5 mL) was degassed and purged with N₂ for 3 times. Then the mixture was stirred at 100° C., for 2 hrs under N₂ atmosphere. On completion, the reaction mixture was diluted with H₂O (20 mL) and extracted with EA (30 mL×3). The combined organic layers were dried over Na₂SO₄ filtered and the filtrate was concentrated under reduced pressure to give a residue. The residue was purified by column chromatography (SiO₂. Petroleum ether/Ethyl acetate=100/1 to 3/1) to give the title compound (2 g. 76% yield) as a yellow solid and confirmed. LC-MS (ESI) m/z 531.6.

Step 2—2-(6-(tert-butoxycarbonyl)-5-(1-(cyclopentylmethyl)-5-methyl-1H-pyrazol-4-yl) pyridin-2-yl)-1,2,3,4-tetrahydroisoquinoline-8-carboxylic acid. To a solution of methyl 2-(6-(tert-butoxycarbonyl)-5-(1-(cyclopentylmethyl)-5-methyl-1H-pyrazol-4-yl) pyridin-2-yl)-1,2,3,4-tetrahydroisoquinoline-8-carboxylate (2 g, 3.77 mmol) in THF (7 mL) was added LiOH·H₂O (1.58 g, 37.6 mmol) and H₂O (7 mL). The mixture was stirred at 25° C., for 12 hrs. On completion, the reaction mixture was diluted with H₂O (15 mL) and extracted with EA (20 mL×3). The combined organic layers were dried over Na₂SO₄ filtered and the filtrate was concentrated under reduced pressure to give the title compound (2 g) as a yellow solid. LC-MS (ESI) m/z 517.8 (M+H).

Step 3—Tert-butyl-3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinate. To a solution of thiazolo[5,4-b]pyridin-2-amine (760 mg. 5.03 mmol. CAS #31784-70-0) in DMF (30 mL) was added HATU (1.91 g. 5.03 mmol). DIEA (5.00 g, 38.7 mmol), and 2-(6-(tert-butoxycarbonyl)-5-(1-(cyclopentylmethyl)-5-methyl-1H-pyrazol-4-yl) pyridin-2-yl)-1,2,3,4-tetrahydroisoquinoline-8-carboxylic acid (2 g, 3.87 mmol). The mixture was then stirred at 50° C., for 15 hrs. On completion, the reaction mixture was diluted with H₂O (50 mL) and extracted with EA (30 mL×3). The combined organic layers were washed with brine (40 mL×3), dried over Na₂SO₄, filtered and the filtrate was concentrated under reduced pressure to give a residue. The residue was purified by column chromatography (SiO₂. DCM/Ethyl acetate=10/1 to 1/1) to give the title compound (1.8 g, 71% yield) as a yellow solid. LC-MS (ESI) m/z 650.4 (M+H).

Step 4—3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinic acid. To a solution of tert-butyl 3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinate (1.8 g, 2.77 mmol) in DCM (10 mL) was added HCl/dioxane (4 M. 692 uL). The mixture was then stirred at 25° C., for 2 hrs. On completion, the reaction mixture was concentrated under reduced pressure to give the title compound (1.5 g) as a yellow solid. LC-MS (ESI⁺) m/z 594.4 (M+H)⁺.

### 3-6-(N-(3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid (Intermediate GV)

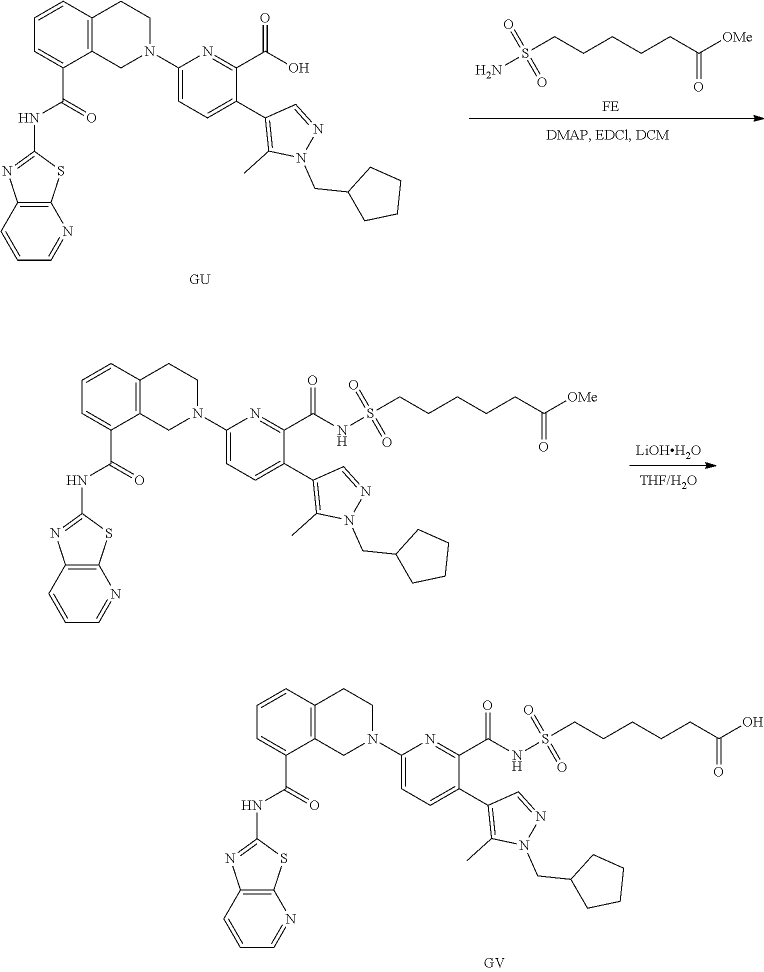 [SMILES: Cc1c(-c2ccc(N3CCc4cccc(C(=O)Nc5nc6cccnc6s5)c4C3)nc2C(=O)O)cnn1CC1CCCC1 ; COC(=O)CCCCCS(N)(=O)=O ; "FE" ; COC(=O)CCCCCS(=O)(=O)NC(=O)c1nc(N2CCc3cccc(C(=O)Nc4nc5cccnc5s4)c3C2)ccc1-c1cnn(CC2CCCC2)c1C ; "LiOH·H2O" ; Cc1c(-c2ccc(N3CCc4cccc(C(=O)Nc5nc6cccnc6s5)c4C3)nc2C(=O)NS(=O)(=O)CCCCCC(=O)O)cnn1CC1CCCC1]

Step 1—Methyl-6-(N-(3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoate. To a solution of methyl 6-sulfamoylhexanoate (282 mg, 1.26 mmol, Intermediate FE), 3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinic acid (500 mg, 842 umol, Intermediate GU) in DCM (10 mL) was added EDCI (322 mg, 1.68 mmol) and DMAP (411 mg, 3.37 mmol). The mixture was stirred at 25° C., for 12 hrs. On completion, the reaction mixture was diluted with H₂O (10 mL) and extracted with EA (15 mL×3). The combined organic layers were dried over Na₂SO₄ filtered and the filtrate was concentrated under reduced pressure to give a residue. The residue was purified by column chromatography (SiO₂, DCM/EA=10/1 to 1/1) to give the title compound (0.35 g, 52% yield) as a yellow solid. LC-MS (ESI⁺) m z 799.3 (M+H)⁺.

Step 2—3-6-(N-(3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid. To a solution methyl 6-(N-(3-(1-(cyclopentylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoate (0.3 g, 375 umol) in THF (5 mL) and H₂O (5 mL) was added LiOH—H₂O (157 mg, 3.75 mmol). The mixture was then stirred at 25° C., for 3 hrs. On completion, the pH of reaction mixture was adjust to acid condition (pH=3-4) with HCl (1 M) and then extracted with EA (15 mL×3). The combined organic layers were dried over Na₂SO₄, filtered and the filtrate was concentrated under reduced pressure to give the title compound (180 mg) as a yellow solid. LC-MS (ESI⁺) m/z 771.3 (M+H)⁺.

### 3-(1-((1S,3S)-Adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinic acid (Intermediate GW)

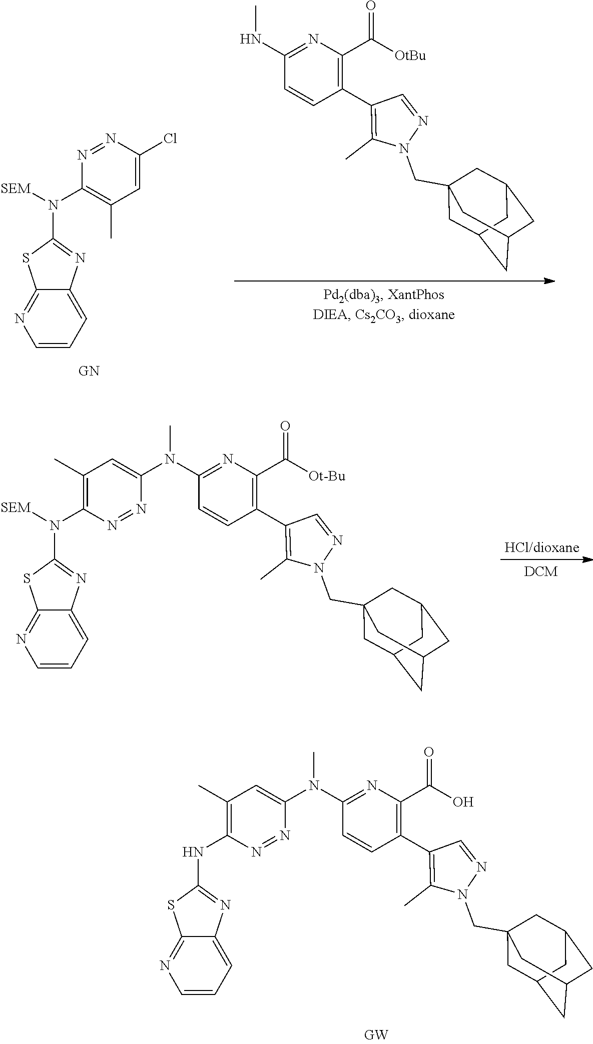 [SMILES: Cc1cc(Cl)nnc1N(COCC[Si](C)(C)C)c1nc2cccnc2s1 ; CNc1ccc(-c2cnn(CC34CC5CC(CC(C5)C3)C4)c2C)c(C(=O)OC(C)(C)C)n1 ; Cc1cc(N(C)c2ccc(-c3cnn(CC45CC6CC(CC(C6)C4)C5)c3C)c(C(=O)OC(C)(C)C)n2)nnc1N(COCC[Si](C)(C)C)c1nc2cccnc2s1 ; Cc1cc(N(C)c2ccc(-c3cnn(CC45CC6CC(CC(C6)C4)C5)c3C)c(C(=O)O)n2)nnc1Nc1nc2cccnc2s1]

Step 1—Tert-butyl 3-(1-((1s,3s)-adamantan-1-ylmethyl)-5-methyl-1H-pyrazol-4-yl)-6-(methyl (5-methyl-6-(thiazolo[5,4-b]pyridin-2-yl((2-(trimethylsilyl)ethoxy)methyl)amino)pyridazin-3-yl)amino)picolinate. To a solution of tert-butyl 3-(1-((1s,3s)-adamantan-1-ylmethyl)-5-methyl-1H-pyrazol-4-yl)-6-(methylamino)picolinate (4.94 g, 11.31 mmol, synthesized via Step 1 of Intermediate FP), N-(6-chloro-4-methylpyridazin-3-yl)-N-((2-(trimethylsilyl)ethoxy)methyl) thiazolo[5,4-b]pyridin-2-amine (6.00 g, 14.7 mmol, Intermediate GN), and Cs₂CO₃ (11.1 g, 33.9 mmol) in dioxane (60 mL) was added Xantphos (1.31 g, 2.26 mmol), Pd₂(dba); (1.04 g, 1.13 mmol), and DIEA (4.39 g, 33.9 mmol, 5.91 mL) under N₂. The mixture was stirred at 120° C., for 12 h. On completion, the reaction mixture was filtered and diluted with water (100 mL), and extracted with EtOAc (120 mL×3). The combined organic layers were washed with brine (40 mL×3), dried over Na₂SO₄ and evaporated. The residue was purified by column chromatography (SiO₂, DCM/EtOAc=20:1 to 10:1) to afford the title compound (9.20 g, 91% yield) as a brown solid. ¹H NMR (400 MHZ, DMSO-d₆) δ=8.35 (dd, J=1.2, 4.8 Hz, 1H), 7.79 (dd, J=1.2, 8.4 Hz, 1H), 7.68 (d, J=0.8 Hz, 1H), 7.65 (d, J=8.8 Hz, 1H), 7.46 (dd, J=4.8, 8.0 Hz, 1H), 7.32 (s, 1H), 7.31 (s, 1H), 5.86 (s, 2H), 3.76-3.71 (m, 4H), 3.64 (s, 3H), 2.37 (s, 3H), 2.18 (s, 3H), 1.94 (br s, 3H), 1.69-1.63 (m, 4H), 1.56 (br d, J=1.6 Hz, 8H), 1.35 (s, 9H), 0.92 (t, J=8.0 Hz, 2H), −0.11 (s, 9H).

Step 2—3-(1-((1S,3S)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinic acid. To a solution of tert-butyl 3-(1-((1s,3s)-adamantan-1-ylmethyl)-5-methyl-1H-pyrazol-4-yl)-6-(methyl (5-methyl-6-(thiazolo[5,4-b]pyridin-2-yl((2-(trimethylsilyl)ethoxy)methyl)amino)pyridazin-3-yl)amino)picolinate (9.20 g, 10.3 mmol) in DCM (25 mL) was added HCl/dioxane (4 M, 25.6 mL). The mixture was then stirred at 40° C., for 6 h. On completion, the reaction mixture was evaporated to afford the title compound (8.00 g, HCl salt) as a yellow solid. LC-MS (ESI⁺) m/z 622.4 (M+H)⁺: ¹H NMR (400 MHZ, DMSO-d₆) δ=8.37 (d, J=4.0 Hz, 1H), 7.99-7.85 (m, 1H), 7.75-7.69 (m, 2H), 7.45 (dd, J=4.8, 8.0 Hz, 1H), 7.39 (s, 1H), 7.35 (br d, J=9.2 Hz, 1H), 3.75 (s, 2H), 3.63 (br s, 3H), 2.39 (s, 3H), 2.19 (s, 3H), 1.94 (br s, 3H), 1.69-1.64 (m, 3H), 1.59 (br s, 2H), 1.55 (br s, 7H).

### 6-[3-[1-(1-adamantylmethyl)-5-methyl-pyrazol-4-yl]-6-[methyl-[5-methyl-6-(thiazolo[5,4-b]pyridin-2-ylamino)pyridazin-3-yl]amino]pyridine-2-carbonyl]sulfamoyl]hexanoic acid (Intermediate GX)

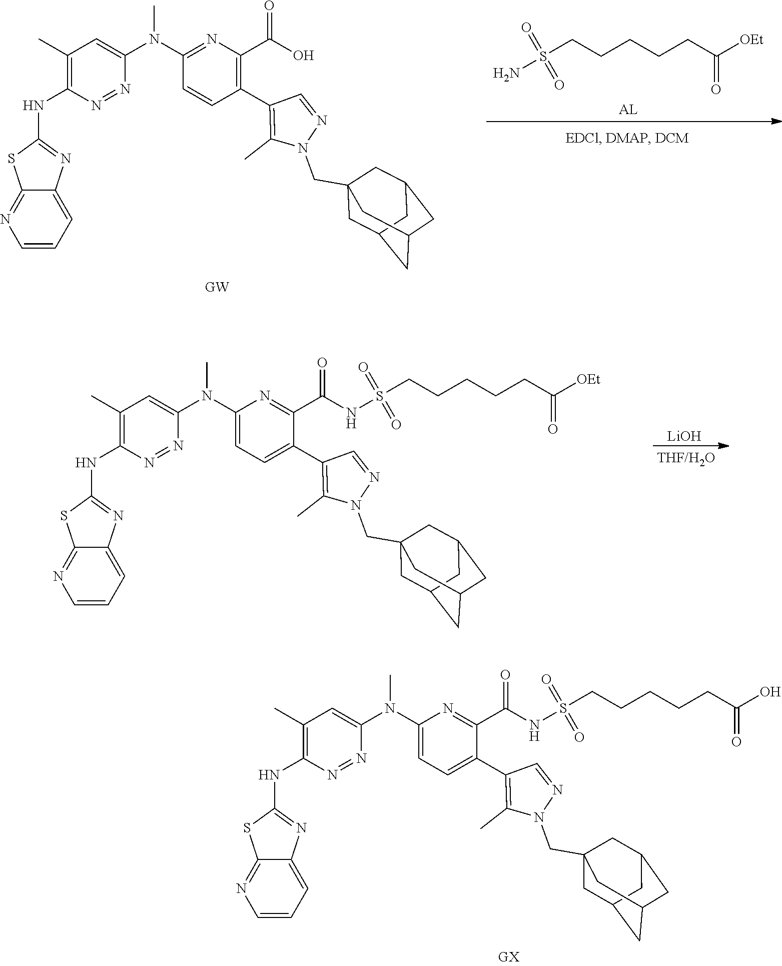 [SMILES: Cc1cc(N(C)c2ccc(-c3cnn(CC45CC6CC(CC(C6)C4)C5)c3C)c(C(=O)O)n2)nnc1Nc1nc2cccnc2s1 ; CCOC(=O)CCCCCS(N)(=O)=O ; "AL" ; CCOC(=O)CCCCCS(=O)(=O)NC(=O)c1nc(N(C)c2cc(C)c(Nc3nc4cccnc4s3)nn2)ccc1-c1cnn(CC23CC4CC(CC(C4)C2)C3)c1C ; "LiOH" ; Cc1cc(N(C)c2ccc(-c3cnn(CC45CC6CC(CC(C6)C4)C5)c3C)c(C(=O)NS(=O)(=O)CCCCCC(=O)O)n2)nnc1Nc1nc2cccnc2s1]

Step 1—Ethyl 6-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoate. To a solution of 3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinic acid (2.00 g, 2.73 mmol, HCl salt, Intermediate GW), ethyl 6-sulfamoylhexanoate (1.83 g, 8.20 mmol, Intermediate AL) in DCM (20 mL) was added EDCI (1.05 g, 5.47 mmol) and DMAP (1.34 g, 11.0 mmol). The mixture was then stirred at 25° C., for 12 h. On completion, the reaction mixture was diluted with water (15 mL) and extracted with DCM (20 mL×3). The combined organic layers were washed with brine (10 mL×3), dried over Na₂SO₄ and evaporated. The crude product was purified by reversed-phase HPLC (ACN/0.1% FA=80%) to afford the title compound (740 mg, 33% yield) as a yellow solid. ¹H NMR (400 MHZ, DMSO-d₆) δ=12.00-11.65 (m, 1H), 11.58-10.82 (m, 1H), 8.36 (br d, J=3.2 Hz, 1H), 8.13 (s, 1H), 8.01-7.87 (m, 1H), 7.84 (s, 1H), 7.70 (br d, J=8.4 Hz, 1H), 7.43 (br dd, J=4.8, 7.6 Hz, 1H), 7.39-7.30 (m, 2H), 3.98 (d, J=7.2 Hz, 2H), 3.74 (s, 2H), 3.66 (s, 3H), 2.40 (s, 3H), 2.23-2.16 (m, 5H), 1.94 (br s, 3H), 1.68-1.63 (m, 3H), 1.61-1.53 (m, 11H), 1.46-1.40 (m, 2H), 1.35-1.21 (m, 3H), 1.12 (t, J=7.2 Hz, 3H).

Step 2—6-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoic acid. To a solution of ethyl 6-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)hexanoate (740 mg, 895 umol) in THF (4 mL) and H₂O (4 mL) was added LiOH·H₂O (188 mg, 4.47 mmol). The mixture was then stirred at 25° C., for 12 h. On completion, the reaction mixture was diluted with water (5 mL) and the pH was adjusted to 4˜3 by addition 2 M HCl, then extracted with DCM (15 mL×3). The combined organic layers were washed with brine (5 mL×3), dried over Na₂SO₄ and evaporated to afford the title compound (720 mg) as a yellow solid. LC-MS (ESI⁺) m/z 799.4 (M+H)⁺.

### 1-(4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylic acid (Intermediate GY)

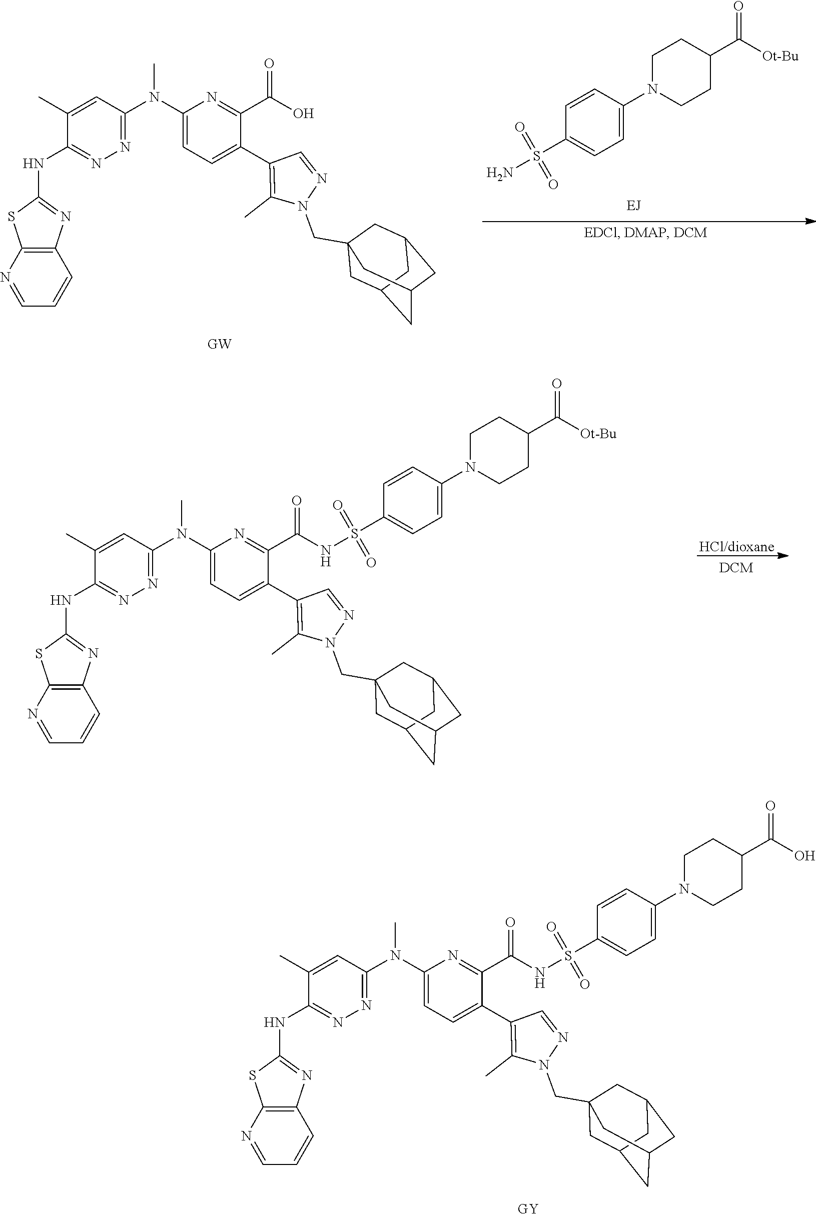 [SMILES: Cc1cc(N(C)c2ccc(-c3cnn(CC45CC6CC(CC(C6)C4)C5)c3C)c(C(=O)O)n2)nnc1Nc1nc2cccnc2s1 ; CC(C)(C)OC(=O)C1CCN(c2ccc(S(N)(=O)=O)cc2)CC1 ; Cc1cc(N(C)c2ccc(-c3cnn(CC45CC6CC(CC(C6)C4)C5)c3C)c(C(=O)NS(=O)(=O)c3ccc(N4CCC(C(=O)OC(C)(C)C)CC4)cc3)n2)nnc1Nc1nc2cccnc2s1 ; "HCl/dioxane" ; Cc1cc(N(C)c2ccc(-c3cnn(CC45CC6CC(CC(C6)C4)C5)c3C)c(C(=O)NS(=O)(=O)c3ccc(N4CCC(C(=O)O)CC4)cc3)n2)nnc1Nc1nc2cccnc2s1]

Step 1—Tert-butyl 1-(4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylate. To a solution of 3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinic acid (500 mg, 684 umol, HCl, Intermediate GW) and tert-butyl 1-(4-sulfamoylphenyl)piperidine-4-carboxylate (465 mg, 1.37 mmol, Intermediate EJ) in DCM (10 mL) was added DMAP (418 mg, 3.42 mmol) and EDCI (542 mg, 2.74 mmol). On completion, the reaction mixture was quenched with H₂O (15 mL), and then extracted with DCM (40 mL×3). The combined organic layers were washed with brine (15 mL×3), dried over Na₂SO₄, filtered and concentrated under reduced pressure to give a residue. The crude product was purified by reversed-phase HPLC (ACN/0.1% FA=70%) to afford the title compound (182 mg, 26% yield) as a yellow solid. ¹H NMR (400 MHZ, DMSO-d₆) δ=11.92-11.77 (m, 1H), 11.46-11.10 (m, 1H), 8.36 (br s, 1H), 8.04-7.91 (m, 1H), 7.85 (s, 1H), 7.67 (br d, J=8.4 Hz, 1H), 7.61 (d, J=9.2 Hz, 2H), 7.44 (br dd, J=4.8, 7.8 Hz, 1H), 7.31 (br d, J=8.4 Hz, 1H), 7.23 (s, 1H), 6.93 (br d, J=9.2 Hz, 2H), 3.76 (br d, J=13.2 Hz, 2H), 3.64 (s, 5H), 2.93-2.87 (m, 2H), 2.44 (br s, 3H), 1.95 (br s, 3H), 1.91 (s, 2H), 1.76 (br d, J=10.4 Hz, 2H), 1.70-1.63 (m, 4H), 1.60-1.53 (m, 5H), 1.50 (br s, 7H), 1.38 (s, 9H).

Step 2—1-(4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylic acid. To a solution of tert-butyl 1-(4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylate (182 mg, 116 umol) in DCM (1.50 mL) was added HCl/dioxane (4 M, 1.50 mL). The mixture was then stirred at 25° C., for 12 h. On completion, the reaction mixture was filtered and concentrated under reduced pressure to afford the title compound (108 mg, 90% yield, HCl) as a brown solid. ¹H NMR (400 MHZ, DMSO-d₆) δ=11.93 (br d, J=1.2 Hz, 1H), 8.41 (d, J=4.4 Hz, 1H), 7.94 (s, 1H), 7.75 (d, J=8.4 Hz, 1H), 7.64 (d, J=8.8 Hz, 2H), 7.42 (d, J=8.8 Hz, 1H), 7.38 (s, 1H), 7.25 (d, J=2.0 Hz, 2H), 7.12 (s, 1H), 6.98 (d, J=9.2 Hz, 2H), 3.67-3.64 (m, 4H), 3.56 (s, 3H), 2.94 (br t, J=11.2 Hz, 2H), 2.45 (s, 3H), 1.95 (br s, 3H), 1.90 (s, 2H), 1.83 (br d, J=10.8 Hz, 2H), 1.71-1.62 (m, 4H), 1.57 (br d, J=11.6 Hz, 6H), 1.50 (br s, 5H), 1.23 (s, 1H).

### 4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)butanoic acid (Intermediate GZ)

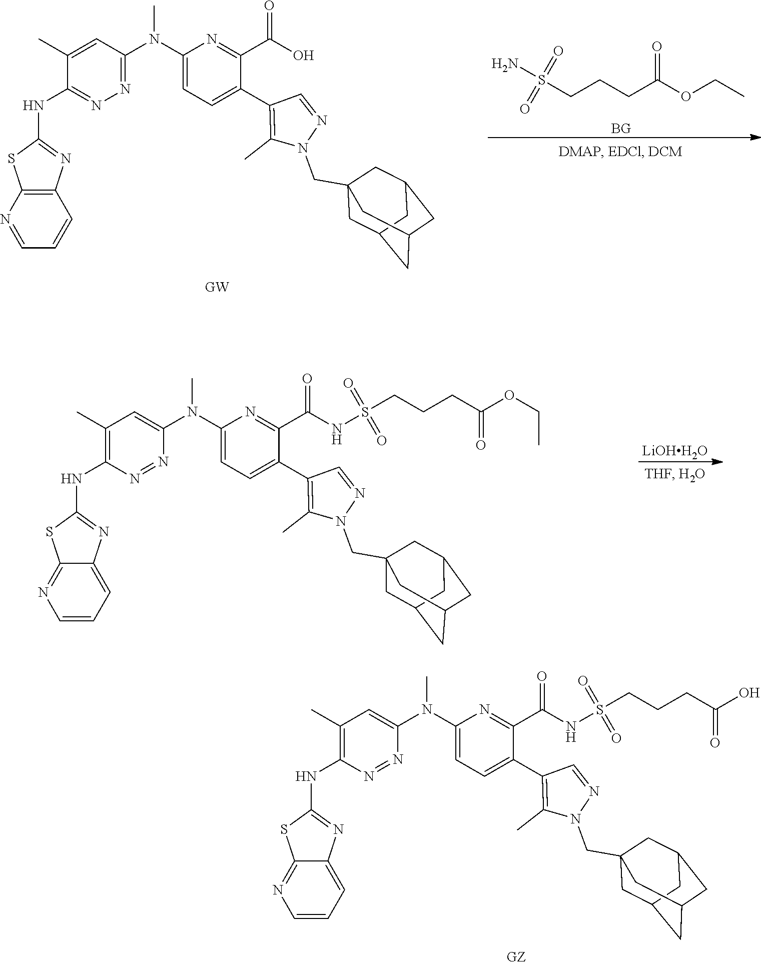

Step 1—Ethyl 4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)butanoate. To a solution of 3-(1-((1s,3s)-adamantan-1-ylmethyl)-5-methyl-IH-pyrazol-4-yl)-6-(methyl (5-methyl-6-(thiazolo[5,4-b]pyridin-2-ylamino)pyridazin-3-yl)amino)picolinic acid (500 mg, 684 umol, 90.0% purity, HCl, Intermediate GW), ethyl 4-sulfamoylbutanoate (400 mg, 2.05 mmol, Intermediate BG), DMAP (334 mg, 2.73 mmol), and EDCI (262 mg, 1.37 mmol) in DCM (5 mL) was degassed and purged with N₂ three times. Then the mixture was stirred at 25° C., for 12 h under N₂ atmosphere. On completion, the reaction mixture was quenched with H₂O (10 mL), and then extracted with DCM (10 mL×3). The combined organic layers were washed with brine (5 mL×3), dried over Na₂SO₄, filtered and concentrated under reduced pressure to give a residue. The residue was purified by reversed-phase HPLC (0.1% FA condition, 80%-90%) to give the title compound (270 mg, 49% yield) as a yellow solid. LC-MS (ESI⁺) m/z 799.6 (M+H)⁺.

Step 2—4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)butanoic acid. A solution of ethyl 4-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)butanoate (270 mg, 338 umol) and LiOH·H₂O (70.9 mg, 1.69 mmol) in THF (1.4 mL) and H₂O (1.4 mL) was degassed and purged with N₂ for 3 times. Then the mixture was stirred at 25° C., for 1 h under N₂ atmosphere. On completion, the reaction mixture was quenched with H₂O (2 mL), and then extracted with DCM (3 mL×3). The combined aqueous phase was treated with HCl (2M) to adjusted pH=3˜4, then the mixture was extracted by DCM (2 mL×3). The combined organic phase was concentrated under reduced pressure to give the title compound (270 mg) as a yellow solid. LC-MS (ESI⁺) m/z 771.6 (M+H)⁺.

### 5-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)pentanoic acid (Intermediate HA)

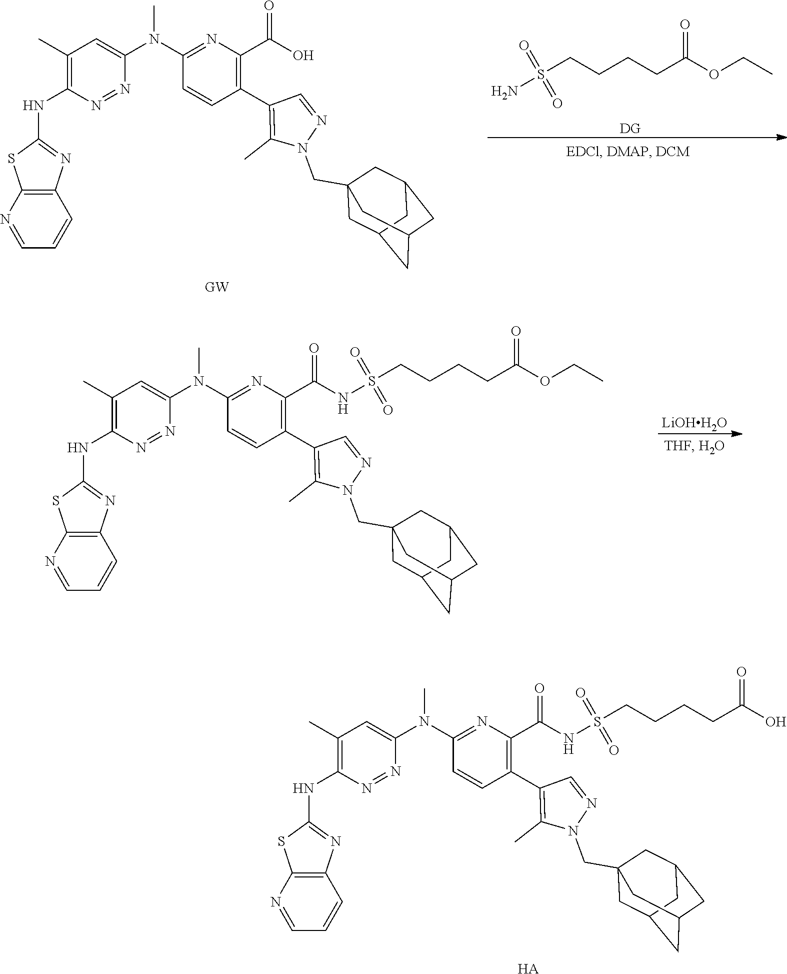

Step 1—Ethyl 5-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)pentanoate. To a solution of 3-[1-(1-adamantylmethyl)-5-methyl-pyrazol-4-yl]-6-[methyl-[5-methyl-6-(thiazolo[5,4-b]pyridin-2-ylamino)pyridazin-3-yl]amino]pyridine-2-carboxylic acid (1.00 g, 1.37 mmol, HCl, Intermediate GW), ethyl 5-sulfamoylpentanoate (858 mg, 4.10 mmol, Intermediate DG) in DCM (5 mL) was added EDCI (1.05 g, 5.46 mmol) and DMAP (1.34 mg, 0.11 mol). Then the mixture was stirred at 25-40° C., for 24 h. On completion, the reaction mixture was quenched with H₂O (15 mL) and extracted with DCM (10 mL×3). The combined organic layer was dried over Na₂SO₄ and evaporated. The crude product was purified by reversed-phase HPLC (0.1% FA condition, 5%-85%, 40 min) to give the title compound (430 mg, 39% yield) as an orange solid.

Step 2—5-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)pentanoic acid. To a solution of ethyl 5-(N-(3-(1-((1s, 3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)pentanoate (420 mg, 517 umol) in THF (3 mL) and H₂O (1 mL) was added LiOH·H₂O (108 mg, 2.58 mmol). The mixture was then stirred at 25-40° C., for 12.5 h. On completion, the reaction mixture was quenched with water (4 mL) at 20° C., and extracted with DCM (3 mL×2). Then the aqueous was adjust to pH=3˜4 and extracted with DCM (4 mL×4). The organic layers were washed with brine (15 mL×2), dried over Na₂SO₄, filtered and concentrated under reduced pressure to give the title compound (340 mg, 84% yield) as a yellow solid. LC-MS (ESI⁺) m/z 785.6 (M+H)⁺.

### 6-((6-(Benzo[d]thiazol-2-ylamino)-5-methylpyridazin-3-yl)(methyl)amino)-3-(5-methyl-1-neopentyl-1H-pyrazol-4-yl)picolinic acid (Intermediate HB)

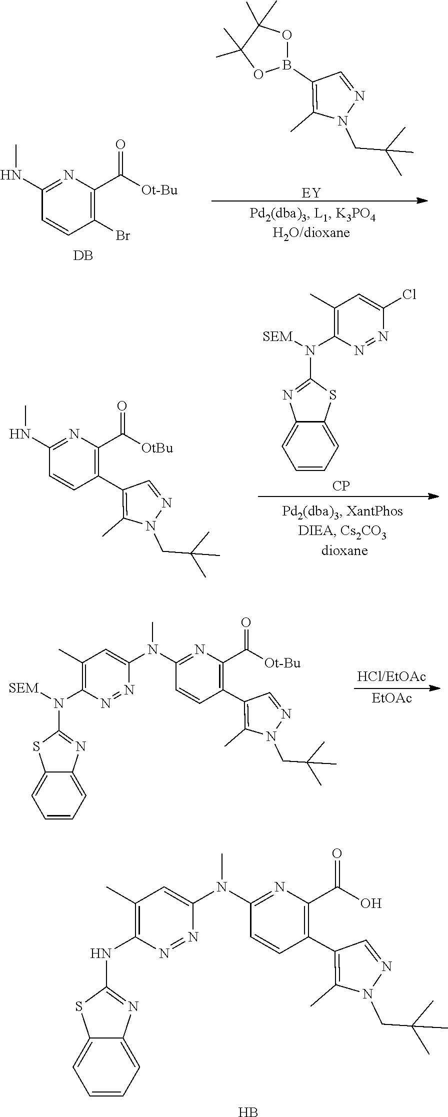

Step 1—Tert-butyl 3-(5-methyl-1-neopentyl-1H-pyrazol-4-yl)-6-(methylamino)picolinate. To a solution tert-butyl 3-bromo-6-(methylamino)picolinate (4 g, 13.9 mmol, Intermediate DB) and 5-methyl-1-neopentyl-4-(4,4,5,5-tetramethyl-1,3,2-dioxaborolan-2-yl)-1H-pyrazole (5.04 g, 18.1 mmol, Intermediate EY) in dioxane (50 mL) and H₂O (20 mL) was added Pd₂(dba); (637 mg. 696 umol), (1S,3R,5R,7S)-1,3,5,7-tetramethyl-8-phenyl-2,4,6-trioxa-8-phosphatricyclo[3.3.1.1^{3,7}]decane (814.37 mg, 2.79 mmol, CAS #97739-46-3, L1) and K₃PO₄ (5.91 g, 27.86 mmol). Then the mixture was stirred at 100° C., for 4 h under N₂. On completion, the reaction was quenched with H₂O (20 mL) and extracted with DCM (20 mL×3). The combined organic layer was dried over Na₂SO₄ and evaporated. The residue was purified by silica gel column chromatography (PE:EA=1:0˜1:1) to give the title compound (5.7 g, 99% yield) as a white solid. LC-MS (ESI) m/z 359.4 (M+H).

Step 2—Tert-butyl 6-((6-(benzo[d]thiazol-2-yl((2-(trimethylsilyl)ethoxy)methyl)amino)-5-methylpyridazin-3-yl)(methyl)amino)-3-(5-methyl-1-neopentyl-1H-pyrazol-4-yl)picolinate. To a solution of N-(6-chloro-4-methylpyridazin-3-yl)-N-((2-(trimethylsilyl)ethoxy)methyl)benzo[d]thiazol-2-amine (2.04 g, 5.02 mmol, Intermediate CP) and tert-butyl 3-(5-methyl-1-neopentyl-1H-pyrazol-4-yl)-6-(methylamino)picolinate (1.2 g, 3.35 mmol) in dioxane (17 mL) was added Xantphos (387.38 mg, 669.50 umol, CAS #161265-03-8), Cs₂CO₃ (3.27 g, 10.0 mmol), DIEA (1.30 g, 10.0 mmol) and Pd₂(dba); (306 mg, 334 umol, CAS #51364-51-3) at 20° C., under nitrogen flow. Then the reaction was stirred at 100° C. for 10 h under nitrogen atmosphere. On completion, the reaction was concentrated to give a residue. The residue was purified by column chromatography on silica gel (eluted with petroleum ether:ethyl acetate=100:1 to 100:30) to give the title compound (1.1 g, 45% yield) as a yellow solid. ¹H NMR (400 MHZ, CDCl₃) ô=7.64-7.57 (m, 2H), 7.54-7.50 (m, 2H), 7.45-7.40 (m, 2H), 7.29-7.23 (m, 1H), 7.18 (d, J=8.4 Hz, 1H), 5.91 (s, 2H), 3.93 (s, 2H), 3.90-3.85 (m, 3H), 3.85-3.79 (m, 2H), 2.45 (s, 3H), 2.27 (s, 3H), 1.48 (s, 9H), 1.13-1.07 (m, 9H), 0.03-0.06 (m, 11H).

Step 3—6-((6-(Benzo[d]thiazol-2-ylamino)-5-methylpyridazin-3-yl)(methyl)amino)-3-(5-methyl-1-neopentyl-1H-pyrazol-4-yl)picolinic acid. To a solution of tert-butyl 6-((6-(benzo[d]thiazol-2-yl((2-(trimethylsilyl)ethoxy)methyl)amino)-5-methylpyridazin-3-yl)(methyl)amino)-3-(5-methyl-1-neopentyl-1H-pyrazol-4-yl)picolinate (1.1 g, 1.51 mmol) in EtOAc (10 mL) was added HCl/EtOAc (4 M, 20 mL) at 20° C., under nitrogen flow. Then the reaction was stirred at 20° C., for 10 h under nitrogen atmosphere. On completion, the reaction was concentrated to give the title compound (1.12 g) as a brown solid. LC-MS (ESI) m/z 543.2 (M+H).

### 1-(4-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)phenyl)piperidine-4-carboxylic acid (Intermediate HC)

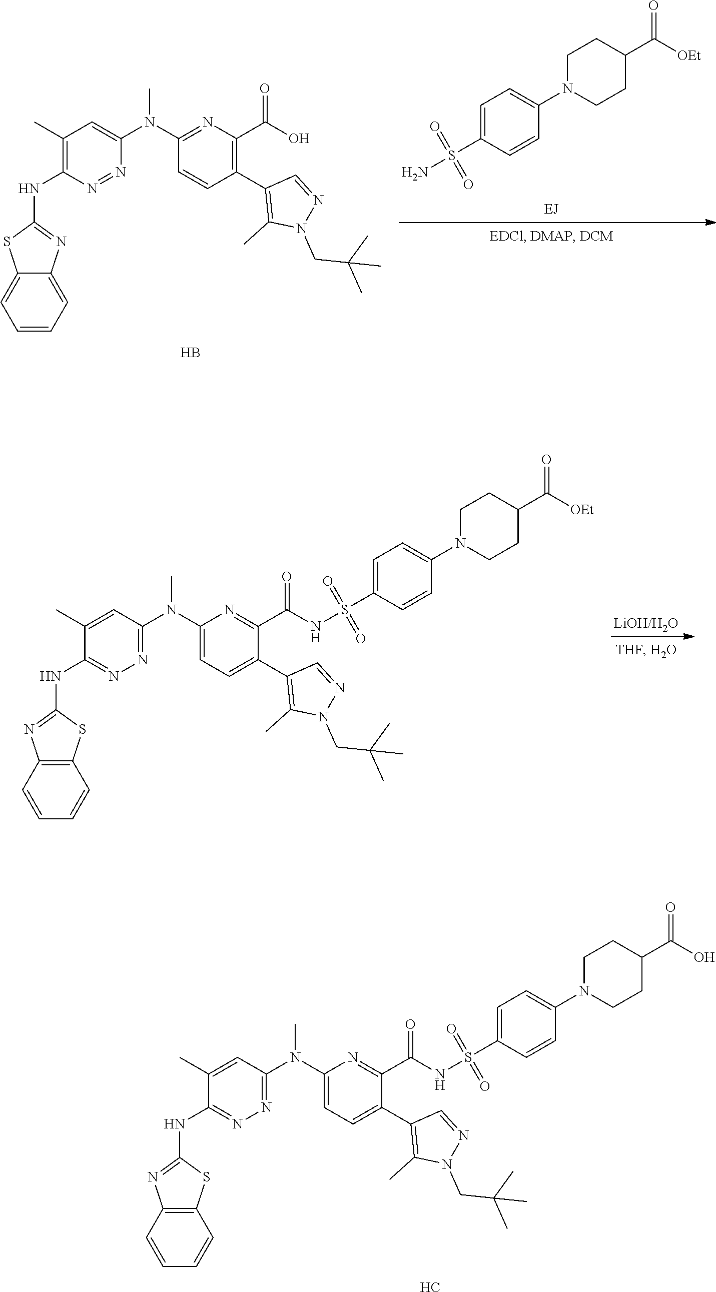

Step 1—Ethyl 1-(4-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)phenyl)piperidine-4-carboxylate. To a solution of ethyl 1-(4-sulfamoylphenyl)piperidine-4-carboxylate (311 mg, 995 umol, Intermediate EJ) and 6-((6-(benzo[d]thiazol-2-ylamino)-5-methylpyridazin-3-yl)(methyl)amino)-3-(5-methyl-1-neopentyl-1H-pyrazol-4-yl)picolinic acid (360 mg, 663 umol, Intermediate HB) in DCM (3.5 mL) was added DMAP (648 mg, 2.65 mmol), EDCI (509 mg, 1.33 mmol) and 4 Å molecular sieves (360 mg). The mixture was stirred at 25° C., for 36 h. On completion, the reaction mixture was quenched with H₂O (5 mL), and then extracted with DCM (15 mL×3). The combined organic layers were washed with brine (5 mL×3), dried over Na₂SO₄, filtered and concentrated under reduced pressure to give a residue. The crude product was purified by reversed-phase HPLC (ACN/0.1% FA=80%) to afford the title compound (162 mg, 27% yield) as a yellow solid. ¹H NMR (400 MHZ, DMSO-d₆) δ=12.08-11.69 (m, 1H), 7.88 (br dd, J=2.0, 3.0 Hz, 1H), 7.83 (s, 1H), 7.69-7.57 (m, 4H), 7.39 (br t, J=7.6 Hz, 1H), 7.32-7.25 (m, 2H), 7.24-7.19 (m, 1H), 6.93 (br d, J=9.2 Hz, 2H), 3.79 (br s, 1H), 3.75 (s, 3H), 3.63 (s, 3H), 3.60 (br t, J=6.0 Hz, 4H), 2.99-2.82 (m, 3H), 2.43 (br s, 3H), 1.85-1.73 (m, 6H), 1.58-1.49 (m, 2H), 0.90 (s, 9H).

Step 2—1-(4-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)phenyl)piperidine-4-carboxylic acid. To a solution of ethyl 1-(4-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)phenyl)piperidine-4-carboxylate (162 mg, 194 umol) in THF (2 mL) and H₂O (1 mL) was added LiOH·H₂O (40.6 mg, 968 umol). The mixture was stirred at 25° C., for 12 hr. On completion, the reaction mixture was filtered and quenched with H₂O (5 mL) and extracted with DCM (15 mL×2), and the pH was adjusted to 4˜5 with 2 M HCl. The solution was extracted with DCM (15 mL×2), and the combined organic layers were washed with brine (5 mL×3), dried over Na₂SO₄, filtered and concentrated under reduced pressure to afford the title compound (150 mg, 85% yield) as a yellow solid. ¹H NMR (400 MHZ, DMSO-d₆) δ=11.90-11.74 (m, 1H), 7.88 (br d, J=7.6 Hz, 1H), 7.83 (s, 1H), 7.70-7.59 (m, 3H), 7.57 (br dd, J=1.2, 6.3 Hz, 1H), 7.39 (br t, J=7.6 Hz, 1H), 7.31 (br d, J=8.8 Hz, 1H), 7.26-7.23 (m, 1H), 7.23-7.17 (m, 1H), 6.95 (br d, J=8.4 Hz, 2H), 3.79 (br d, J=13.2 Hz, 2H), 3.74 (s, 2H), 3.64 (s, 3H), 2.91 (br t, J=11.6 Hz, 2H), 2.43 (br s, 3H), 1.88 (br s, 1H), 1.60-1.48 (m, 3H), 1.23 (br s, 2H), 1.17 (br s, 2H), 0.89 (s, 9H).

### 6-(Methyl (5-methyl-6-(thiazolo[5,4-b]pyridin-2-ylamino)pyridazin-3-yl)amino)-3-(5-methyl-1-neopentyl-1H-pyrazol-4-yl)picolinic acid (Intermediate HD)

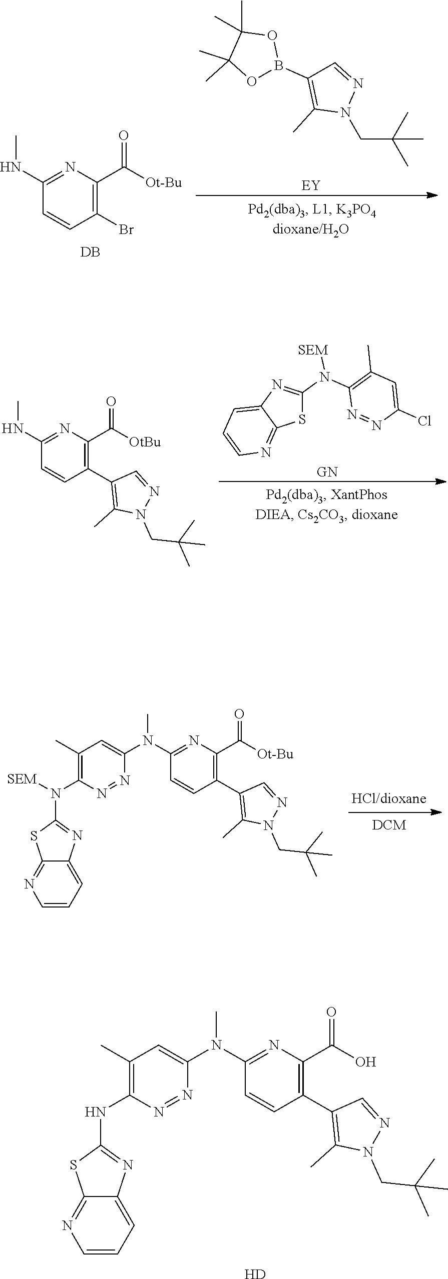

Step 1—Tert-butyl 3-(5-methyl-1-neopentyl-1H-pyrazol-4-yl)-6-(methylamino)picolinate. To a solution tert-butyl 3-bromo-6-(methylamino)picolinate (4.00 g, 13.9 mmol, Intermediate DB) and 5-methyl-1-neopentyl-4-(4,4,5,5-tetramethyl-1,3,2-dioxaborolan-2-yl)-1H-pyrazole (5.04 g, 18.1 mmol, Intermediate EY) in dioxane (50 mL) and H₂O (20 mL) was added Pd₂(dba); (638 mg, 697 umol) and (1S,3R,5R,7S)-1,3,5,7-tetramethyl-8-phenyl-2,4,6-trioxa-8-phosphatricyclo[3.3.1.1^{3,7}]decane (814 mg, 2.79 mmol, L1) and K₃PO₄ (5.91 g, 27.9 mmol). Then the mixture was stirred at 100° C., for 4 h under N₂. On completion, the reaction mixture was quenched with H₂O (20 mL) and extracted with DCM (20 mL×3). The combined organic layer was dried over Na₂SO₄ and evaporated. The residue was purified by silica gel column chromatography (Petroleum ether:Ethyl acetate=1:0˜1:1) to give the title compound (5.70 g, 99% yield) as an orange solid. ¹H NMR (400 MHZ, DMSO-d₆) δ=7.28 (d, J=8.8 Hz, 1H), 7.22 (s, 1H), 6.71 (q, J=4.8 Hz, 1H), 6.53 (d, J=8.8 Hz, 1H), 3.93 (s, 1H), 3.84 (s, 2H), 2.76 (d, J=4.8 Hz, 3H), 2.10 (s, 3H), 1.28 (s, 9H), 1.07 (s, 8H).

Step 2—Tert-butyl 6-(methyl (5-methyl-6-(thiazolo[5,4-b]pyridin-2-yl((2-(trimethylsilyl)ethoxy)methyl)amino)pyridazin-3-yl)amino)-3-(5-methyl-1-neopentyl-1H-pyrazol-4-yl)picolinate. To a solution of N-(6-chloro-4-methylpyridazin-3-yl)-N-((2-(trimethylsilyl)ethoxy)methyl) thiazolo[5,4-b]pyridin-2-amine (1.80 g, 3.40 mmol, Intermediate GN) and tert-butyl 3-(5-methyl-1-neopentyl-1H-pyrazol-4-yl)-6-(methylamino)picolinate (1.00 g, 2.43 mmol) in dioxane (20 mL) was added Xantphos (422 mg. 729 umol), DIEA (942 mg, 7.29 mmol, 1.27 mL), Pd₂(dba); (335 mg, 365 umol), and Cs₂CO₃ (2.38 g, 7.29 mmol) under N₂. The mixture was then stirred at 120° C., for 12 hr. On completion, the reaction mixture was quenched with H₂O (20 mL) and extracted with DCM (20 mL×3). The combined organic layer was then dried over Na₂SO₄ and evaporated. The residue was purified by silica gel column chromatography (Petroleum ether:Ethyl acetate=1:0˜1:1) to give the title compound (1.05 g, 59% yield) as an orange solid. ¹H NMR (400 MHZ, DMSO-d₆) ò=8.35 (d, J=4.8 Hz, 1H), 7.79 (d, J=8.4 Hz, 1H), 7.69-7.62 (m, 1H), 7.46 (dd, J=4.8, 8.4 Hz, 1H), 7.36-7.24 (m, 3H), 5.86 (s, 2H), 3.88 (s, 2H), 3.73 (br t, J=8.0 Hz, 2H), 3.64 (s, 3H), 2.37 (s, 3H), 2.19 (s, 3H), 1.34 (s, 9H), 0.99-0.90 (m, 11H), −0.11 (s, 9H).

Step 3—6-(Methyl (5-methyl-6-(thiazolo[5,4-b]pyridin-2-ylamino)pyridazin-3-yl)amino)-3-(5-methyl-1-neopentyl-1H-pyrazol-4-yl)picolinic acid. Tert-butyl 6-(methyl (5-methyl-6-(thiazolo[5,4-b]pyridin-2-yl((2-(trimethylsilyl)ethoxy)methyl)amino)pyridazin-3-yl)amino)-3-(5-methyl-1-neopentyl-1H-pyrazol-4-yl)picolinate (1.00 g, 1.10 mmol) in HCl/dioxane (10 mL) and DCM (10 mL) was stirred at 25° C., for 12 h. On completion, the solution was concentrated and freeze-drying in vacuo to give the title compound (900 mg) as an orange solid. LC-MS (ESI) m/z 544.4 (M+H).

### 1-(4-(N-(6-(methyl (5-methyl-6-(thiazolo[5,4-b]pyridin-2-ylamino)pyridazin-3-yl)amino)-3-(5-methyl-1-neopentyl-1H-pyrazol-4-yl)picolinoyl)sulfamoyl)phenyl)piperidine-4-carboxylic acid (Intermediate HE)

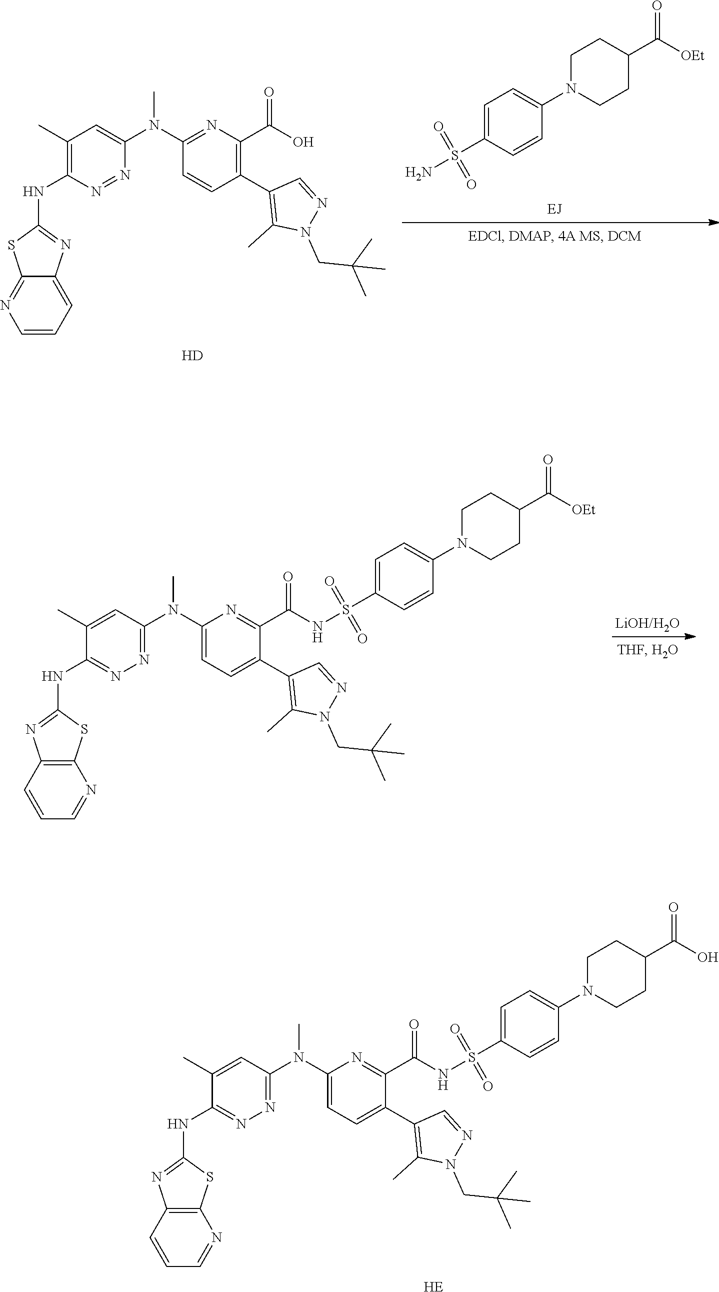

Step 1—Ethyl 1-(4-(N-(6-(methyl (5-methyl-6-(thiazolo[5,4-b]pyridin-2-ylamino)pyridazin-3-yl)amino)-3-(5-methyl-1-neopentyl-1H-pyrazol-4-yl)picolinoyl)sulfamoyl)phenyl)piperidine-4-carboxylate. To a solution of ethyl 1-(4-sulfamoylphenyl)piperidine-4-carboxylate (600 mg, 1.92 mmol, Intermediate EJ), 6-(methyl (5-methyl-6-(thiazolo[5,4-b]pyridin-2-ylamino)pyridazin-3-yl)amino)-3-(5-methyl-1-neopentyl-1H-pyrazol-4-yl)picolinic acid (870 mg, 1.60 mmol, Intermediate HD) in DCM (10 mL) was added EDCI (920 mg, 4.80 mmol), DMAP (1.23 g, 9.60 mmol), and 4 Å molecular sieves (1.00 g, 1.60 mmol). Then the mixture was stirred at 25° C.-40° C., for 18 h. On completion, the reaction mixture was quenched with H₂O (10 mL) and extracted with DCM (15 mL×3). The combined organic layers were washed with brine (4 mL×3), dried over Na₂SO₄ and evaporated. The crude product was purified by reversed-phase HPLC (0.1% FA condition, 5%-62%, 40 min) to give the title compound (695 mg, 47% yield) as a yellow solid. LC-MS (ESI⁺) m/z 838.4 (M+H)⁺.

Step 2—1-(4-(N-(6-(methyl (5-methyl-6-(thiazolo[5,4-b]pyridin-2-ylamino)pyridazin-3-yl)amino)-3-(5-methyl-1-neopentyl-1H-pyrazol-4-yl)picolinoyl)sulfamoyl)phenyl)piperidine-4-carboxylic acid. A solution of ethyl 1-(4-(N-(6-(methyl (5-methyl-6-(thiazolo[5,4-b]pyridin-2-ylamino)pyridazin-3-yl)amino)-3-(5-methyl-1-neopentyl-1H-pyrazol-4-yl)picolinoyl)sulfamoyl)phenyl)piperidine-4-carboxylate (695 mg, 746 umol) and LiOH·H₂O (157 mg, 3.73 mmol) in THF (5 mL) and H₂O (2 mL) was stirred at 25° C., for 2 h. On completion, the reaction mixture was filtered and extracted with DCM (5 mL×2). The aqueous phase pH of the aqueous phase was adjusted to 4˜5 by addition 2M HCl and extracted with DCM (10 mL×8). The combined organic layers were dried over Na₂SO₄ and evaporated to give the title compound (495 mg) as an orange solid. LC-MS (ESI⁺) m/z 810.3 (M+H)⁺.

### 3-(1-(Cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinic acid (Intermediate HF)

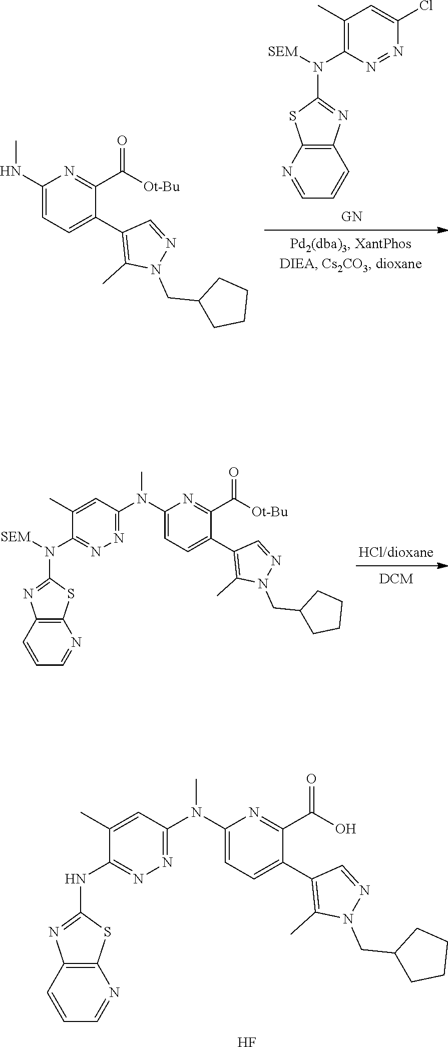

Step 1—Tert-butyl 3-(1-(cyclopentylmethyl)-5-methyl-1H-pyrazol-4-yl)-6-(methyl (5-methyl-6-(thiazolo[5,4-b]pyridin-2-yl((2-(trimethylsilyl)ethoxy)methyl)amino)pyridazin-3-yl)amino)picolinate. To a solution of N-(6-chloro-4-methylpyridazin-3-yl)-N-((2-(trimethylsilyl)ethoxy)methyl) thiazolo[5,4-b]pyridin-2-amine (3.00 g, 7.35 mmol, Intermediate GN) and tert-butyl 3-(1-(cyclopentylmethyl)-5-methyl-1H-pyrazol-4-yl)-6-(methylamino)picolinate (2.48 g, 6.68 mmol, synthesized via Step 1 of Intermediate GQ) in dioxane (40 mL) was added Xantphos (387 mg, 668 umol), Cs₂CO; (6.53 g, 20.0 mmol) and Pd₂(dba); (306 mg, 334 umol). The mixture was then stirred at 120° C., for 12 hr. On completion, the reaction mixture was quenched with H₂O (50 mL), and then extracted with DCM (150 mL×3). The combined organic layers were washed with brine (100 mL×3), dried over Na₂SO₄, filtered and concentrated under reduced pressure to give a residue. The residue was purified by column chromatography (SiO₂, DCM/EA=8/1) to afford the title compound (1.7 g, 31% yield) as a white solid. ¹H NMR (400 MHZ, DMSO-d₆) δ=8.35 (dd, J=1.2, 4.8 Hz, 1H), 7.79 (dd, J=1.2, 8.1 Hz, 1H), 7.68 (d, J=0.8 Hz, 1H), 7.64 (d, J=8.8 Hz, 1H), 7.45 (dd, J=4.8, 8.3 Hz, 1H), 7.33-7.26 (m, 2H), 5.86 (s, 2H), 5.75 (s, 1H), 3.99 (d, J=7.6 Hz, 2H), 3.73 (t, J=8.0 Hz, 2H), 3.64 (s, 3H), 2.89 (s, 1H), 2.73 (s, 1H), 2.36 (s, 3H), 2.18 (s, 3H), 1.66-1.59 (m, 4H), 1.55-1.48 (m, 2H), 1.33 (s, 9H), 1.31-1.27 (m, 2H), 0.95-0.89 (m, 2H), −0.08-0.13 (m, 9H).

Step 2—3-(1-(Cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinic acid. To a solution of tert-butyl 3-(1-(cyclopentylmethyl)-5-methyl-1H-pyrazol-4-yl)-6-(methyl (5-methyl-6-(thiazolo[5,4-b]pyridin-2-yl((2-(trimethylsilyl)ethoxy)methyl)amino)pyridazin-3-yl)amino)picolinate (1.70 g, 2.29 mmol) in DCM (25 mL) was added HCl/dioxane (4 M, 8.59 mL). The mixture was stirred at 25° C., for 16 h. On completion, the reaction mixture was filtered and concentrated under reduced pressure to afford the title compound (1.3 g, 89% yield, HCl) as a yellow solid. ¹H NMR (400 MHZ, DMSO-d₆) δ=8.44 (d, J=4.8 Hz, 1H), 7.97 (br d, J=7.6 Hz, 1H), 7.88-7.80 (m, 2H), 7.54-7.46 (m, 3H), 4.03 (br d, J=7.6 Hz, 3H), 3.69 (s, 3H), 2.45 (s, 3H), 2.25 (s, 3H), 1.67-1.52 (m, 6H), 1.34-1.26 (m, 2H).

### 1-(4-(N-(3-(1-(cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylic acid (Intermediate HG)

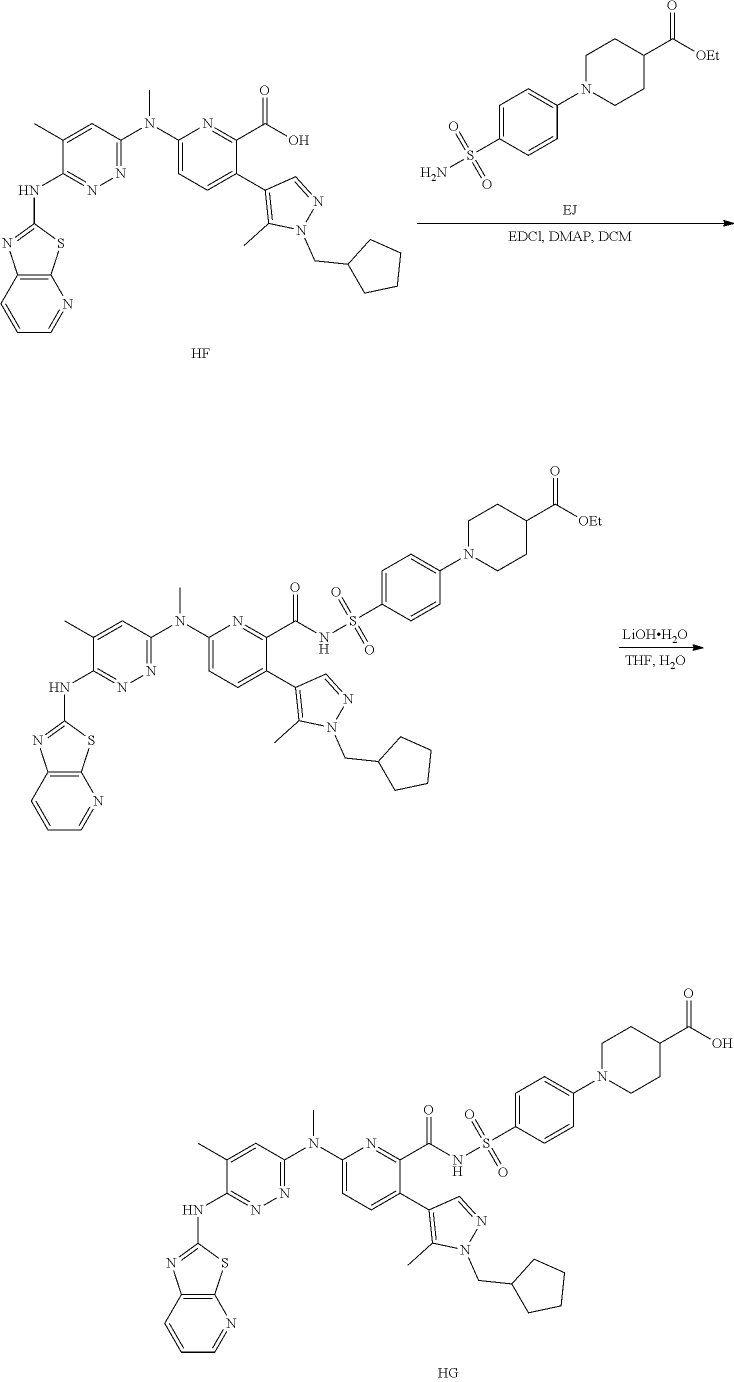

Step 1—Ethyl 1-(4-(N-(3-(1-(cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylate. To a solution of 3-(1-(cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinic acid (400 mg, 628 umol, HCl, Intermediate HF) and ethyl 1-(4-sulfamoylphenyl)piperidine-4-carboxylate (294 mg, 942 umol, Intermediate EJ) in DCM (4 mL) was added DMAP (307 mg, 2.51 mmol) and EDCI (241 mg, 1.26 mmol). The mixture was stirred at 25° C., for 16 h. On completion, the reaction mixture was quenched with H₂O (5 mL), and then extracted with DCM (15 mL×3). The combined organic layers were washed with brine (10 mL×3), dried over Na₂SO₄, filtered and concentrated under reduced pressure to give a residue. The residue was purified by column chromatography (SiO₂, PE/EA=0/1) to afford the title compound (286 mg, 48% yield) as a yellow solid. ¹H NMR (400 MHZ, DMSO-d₆) δ=11.88 (br s, 1H), 11.54-11.14 (m, 1H), 8.38 (br d, J=3.6 Hz, 1H), 8.07-7.90 (m, 1H), 7.87 (br s, 1H), 7.70-7.60 (m, 3H), 7.46-7.41 (m, 1H), 7.33 (br d, J=8.0 Hz, 1H), 7.22 (s, 1H), 6.96 (br d, J=9.2 Hz, 2H), 4.06 (q, J=7.2 Hz, 1H), 4.10-4.02 (m, 1H), 2.21-2.15 (m, 7H), 1.90 (quin, J=7.6 Hz, 10H), 1.42 (s, 3H), 1.31-1.21 (m, 3H), 1.18 (t, J=7.2 Hz, 3H).

Step 2—1-(4-(N-(3-(1-(cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylic acid. To a solution of ethyl 1-(4-(N-(3-(1-(cyclopentylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)phenyl)piperidine-4-carboxylate (230 mg, 271 umol) in THF (3.5 mL) and H₂O (2 mL) was added LiOH·H₂O (56.8 mg, 1.35 mmol). The mixture was then stirred at 25° C., for 12 hr. On completion, the reaction mixture was filtered and quenched with H₂O (10 mL) and extracted with DCM (25 mL×2). The pH was adjusted to 4˜5 by addition 2 M HCl and extracted with DCM (25 mL×2). The combined organic layers were washed with brine (10 mL×3), dried over Na₂SO₄, filtered and concentrated under reduced pressure to afford the title compound (177 mg, 72% yield) as a yellow solid. ¹H NMR (400 MHz, DMSO-d₆) δ=11.86 (br s, 1H), 8.36 (d, J=4.0 Hz, 1H), 7.95-7.89 (m, 1H), 7.86 (s, 1H), 7.66-7.60 (m, 3H), 7.43 (dd, J=4.8, 8.1 Hz, 1H), 7.39-7.26 (m, 2H), 7.20 (s, 1H), 6.96 (br d, J=9.2 Hz, 2H), 3.85 (br d, J=7.2 Hz, 2H), 3.64 (s, 3H), 3.62-3.51 (m, 4H), 2.91 (br t, J=11.2 Hz, 2H), 2.84 (br t, J=6.0 Hz, 1H), 2.69 (s, 1H), 2.43 (s, 3H), 1.75 (td, J=3.2, 6.8 Hz, 3H), 1.64-1.49 (m, 9H), 1.40 (s, 3H), 1.27-1.15 (m, 3H).

### 5-methylpyridin-2-amine (CAS #1603-41-4) (Intermediate HH)

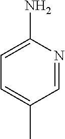

### 3-(1-(Cyclohexylmethyl)-5-methyl-1H-pyrazol-4-yl)-6-(8-(methoxycarbonyl)-3,4-dihydroisoquinolin-2 (1H)-yl)picolinic acid (Intermediate HI)

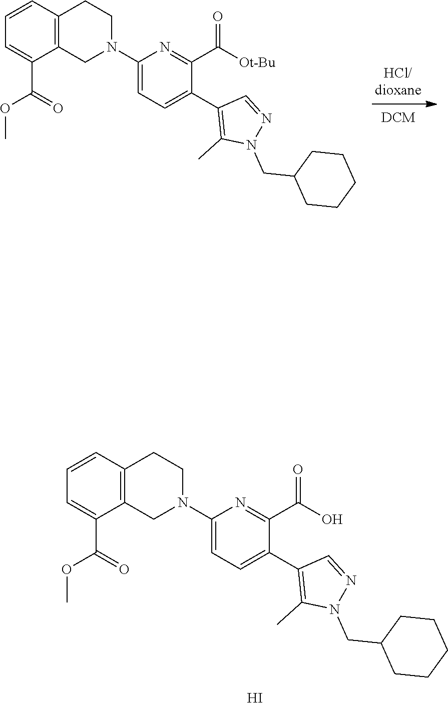

To a solution of methyl 2-[6-tert-butoxycarbonyl-5-[1-(cyclohexylmethyl)-5-methyl-pyrazol-4-yl]-2-pyridyl]-3,4-dihydro-1H-isoquinoline-8-carboxylate (2.3 g, 4.22 mmol, synthesized via Step 1 of Intermediate FF) in DCM (15 mL) was added HCl/dioxane (4 M, 11.5 mL). The mixture was then stirred at 25° C., for 12 h. On completion, the mixture was concentrated in vacuo to give the title compound (2 g, 97% yield) as a yellow solid. LC-MS (ESI⁺) m/z 489.7 (M+H)⁺.

### 6-(N-(3-(1-(cyclohexylmethyl)-5-methyl-1H-pyrazol-4-yl)-6-(8-(methoxycarbonyl)-3,4-dihydroisoquinolin-2 (1H)-yl)picolinoyl)sulfamoyl)hexanoic acid (Intermediate HJ)

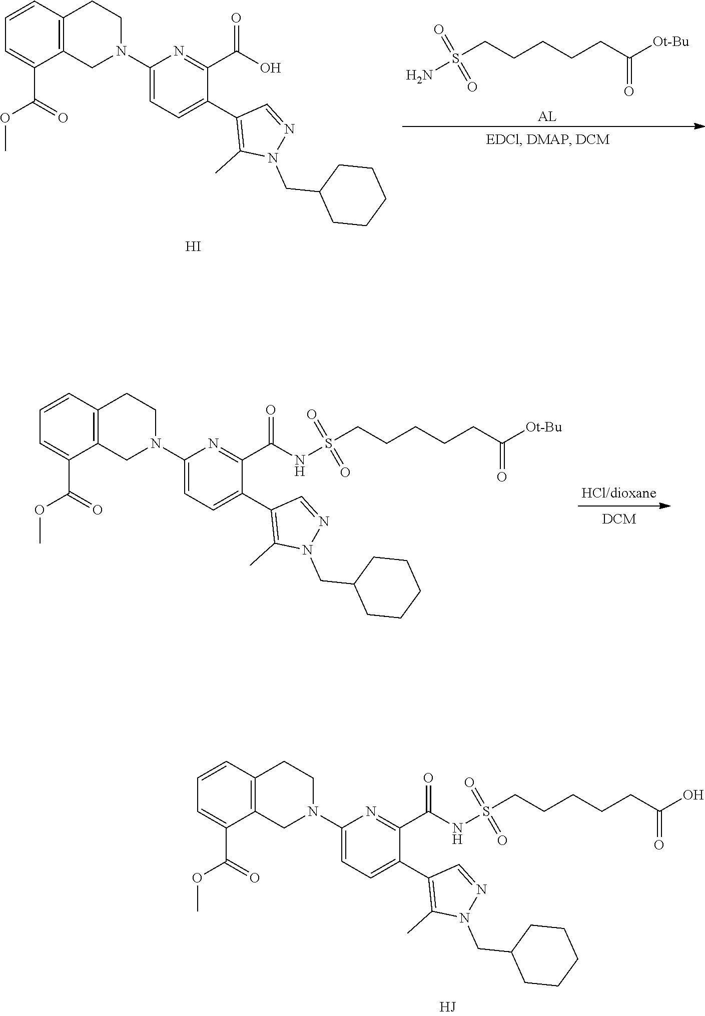

Step 1—Methyl 2-(6-(((6-(tert-butoxy)-6-oxohexyl)sulfonyl) carbamoyl)-5-(1-(cyclohexylmethyl)-5-methyl-1H-pyrazol-4-yl) pyridin-2-yl)-1,2,3,4-tetrahydroisoquinoline-8-carboxylate. To a solution of 3-[1-(cyclohexylmethyl)-5-methyl-pyrazol-4-yl]-6-(8-methoxycarbonyl-3,4-dihydro-IH-isoquinolin-2-yl)pyridine-2-carboxylic acid (2 g, 3.81 mmol, HCl, Intermediate HI), tert-butyl 6-sulfamoylhexanoate (2.87 g, 11.4 mmol. Intermediate AL) in DCM (30 mL) was added EDCI (1.46 g, 7.62 mmol) and DMAP (1.86 g, 15.2 mmol). The mixture was then stirred at 25° C., for 12 h. On completion, the mixture was poured into with H₂O (30 mL), and extracted with DCM mL (30 mL×3). The combined organic layer was then washed with brine (50 mL×3), dried over anhydrous Na₂SO₄, filtered and concentrated under reduced pressure to give a residue. The residue was purified by column chromatography (SiO₂, Petroleum ether:Ethyl acetate=5:1 to Petroleum ether:Ethyl acetate=0:1) to give the title compound (2.5 g, 91% yield) as a white solid. LC-MS (ESI⁺) m/z 722.4 (M+H)⁺.

Step 2—6-(N-(3-(1-(cyclohexylmethyl)-5-methyl-1H-pyrazol-4-yl)-6-(8-(methoxycarbonyl)-3,4-dihydroisoquinolin-2 (1H)-yl)picolinoyl)sulfamoyl)hexanoic acid. To a solution of methyl 2-[6-[(6-tert-butoxy-6-oxo-hexyl)sulfonylcarbamoyl]-5-[1-(cyclohexylmethyl)-5-methyl-pyrazol-4-yl]-2-pyridyl]-3,4-dihydro-1H-isoquinoline-8-carboxylate (500 mg, 692 umol) in DCM (5 mL) was added HCl/dioxane (4 M, 173 uL). The mixture was then stirred at 25° C., for 12 h. On completion, the mixture was concentrated in vacuo to give the title compound (450 mg, 93% yield, HCl) as a yellow solid. LC-MS (ESI⁺) m/z 666.8 (M+H)⁺.

### 2-(5-(1-(Cyclohexylmethyl)-5-methyl-1H-pyrazol-4-yl)-6-(((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)sulfonyl) carbamoyl)pyridin-2-yl)-1,2,3,4-tetrahydroisoquinoline-8-carboxylic acid (Intermediate HK)

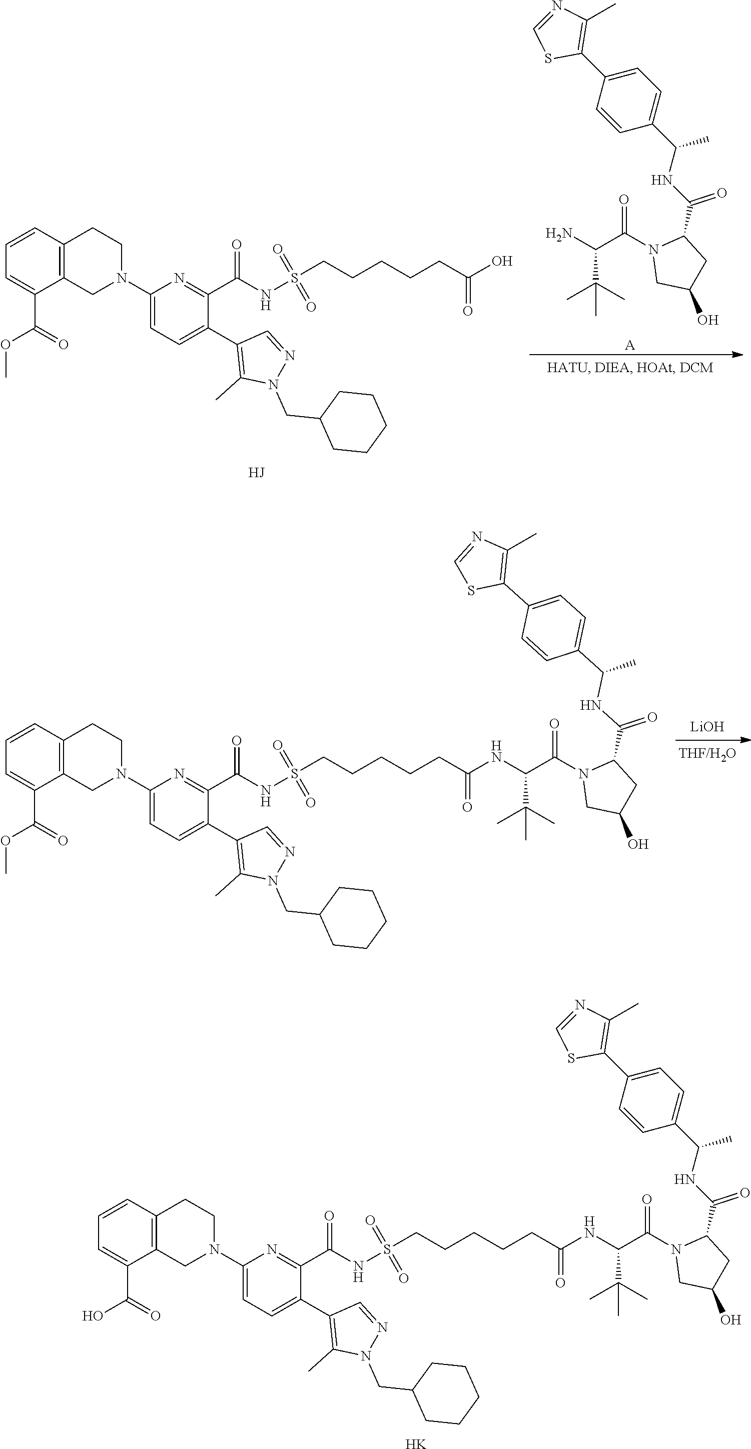

Step 1—Methyl 2-(5-(1-(cyclohexylmethyl)-5-methyl-1H-pyrazol-4-yl)-6-(((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)sulfonyl) carbamoyl)pyridin-2-yl)-1,2,3,4-tetrahydroisoquinoline-8-carboxylate. To a solution of 6-[[3-[1-(cyclohexylmethyl)-5-methyl-pyrazol-4-yl]-6-(8-methoxycarbonyl-3,4-dihydro-1H-isoquinolin-2-yl)pyridine-2-carbonyl]sulfamoyl]hexanoic acid (450 mg, 640 umol, HCl, Intermediate HJ), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (341 mg, 768 umol, Intermediate A) in DCM (0.36 mL) was added HATU (268 mg, 704 umol), HOAt (113 mg, 833 umol, 116.53 uLq), and DIEA (414 mg, 3.20 mmol, 558 uL). The mixture was then stirred at −10° C., for 1 h. On completion, the mixture was poured into H₂O (30 mL), and extracted with DCM mL (30 mL×3). The combined organic layer was washed with brine (50 mL×3), dried over anhydrous Na₂SO₄, filtered and concentrated under reduced pressure to give a residue. The crude product was purified by reversed-phase HPLC (0.1% FA condition) to give the title compound (385 mg, 55% yield) as a yellow solid. LC-MS (ESI⁺) m/z 1192.5 (M+H)⁺.

Step 2—2-(5-(1-(Cyclohexylmethyl)-5-methyl-1H-pyrazol-4-yl)-6-(((6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)sulfonyl) carbamoyl)pyridin-2-yl)-1,2,3,4-tetrahydroisoquinoline-8-carboxylic acid. To a solution of methyl 2-[5-[1-(cyclohexylmethyl)-5-methyl-pyrazol-4-yl]-6-[6-[(IS)-1-[(2S,4R)-4-hydroxy-2-[(1S)-1-[4-(4-methylthiazol-5-yl)phenyl]ethyl]carbamoyl]pyrrolidine-1-carbonyl]-2,2-dimethyl-propyl]amino]-6-oxo-hexyl]sulfonylcarbamoyl]-2-pyridyl]-3,4-dihydro-1H-isoquinoline-8-carboxylate (385 mg, 352 umol) in THF (3 mL) and H₂O (1 mL) was added LiOH·H₂O (73.9 mg, 1.76 mmol). The mixture was then stirred at 25° C., for 12 h. On completion, the mixture was adjusted pH to acidity with HCl and concentrated in vacuo. The mixture was washed with H₂O (3 mL), and extracted with EtOAc mL (3 mL×3). The combined organic layer was washed brine (5 mL×3), dried over anhydrous Na₂SO₄, filtered and concentrated under reduced pressure to give the title compound (350 mg, 92% yield) as a yellow solid. LC-MS (ESI⁺) m/z 1079.8 (M+H)⁺.

### 6-(N-(3-(1-(cyclohexylmethyl)-5-methyl-1H-pyrazol-4-yl)-6-(8-((4-fluorobenzo[d]thiazol-2-yl) carbamoyl)-3,4-dihydroisoquinolin-2 (1H)-yl)picolinoyl)sulfamoyl)hexanoic acid (Intermediate HL)

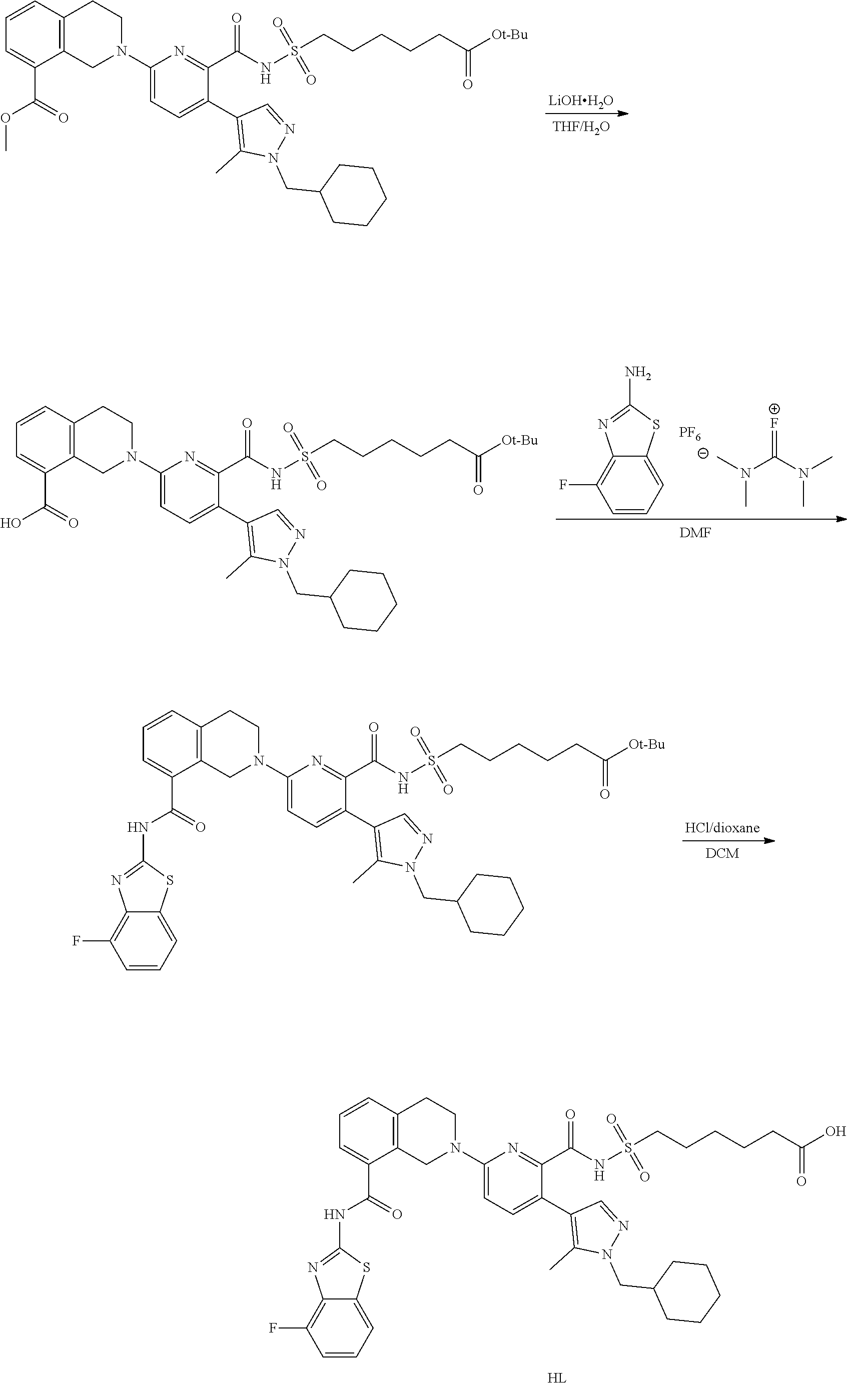

Step 1—2-(6-(((6-(Tert-butoxy)-6-oxohexyl)sulfonyl) carbamoyl)-5-(1-(cyclohexylmethyl)-5-methyl-1H-pyrazol-4-yl) pyridin-2-yl)-1,2,3,4-tetrahydroisoquinoline-8-carboxylic acid. To a solution of methyl 2-[6-[(6-tert-butoxy-6-oxo-hexyl)sulfonylcarbamoyl]-5-[1-(cyclohexylmethyl)-5-methyl-pyrazol-4-yl]-2-pyridyl]-3,4-dihydro-1H-isoquinoline-8-carboxylate (0.5 g, 693 umol, synthesized via Step 1 of Intermediate HJ) in THF (2.5 mL) and H₂O (2.5 mL), was added LiOH·H₂O (145 mg, 3.46 mmol). The mixture was then stirred at 20° C., for 16 hrs. On completion, the reaction was diluted with water (50 mL), acidified with HCl (2 N) to pH=5, then it was extracted with DCM (20 mL×3). The combined organic layer was washed with brine (30 mL), dried over Na₂SO₄, filtered and concentrated in vacuo. The crude was purified by silica gel column chromatography: Petroleum: EtOAc from 10:1 to 0:1 to afford the title compound (350 mg, 61% yield) as a brown oil. LC-MS (ESI⁺) m/z 708.6 (M+H)⁺.

Step 2—Tert-butyl 6-(N-(3-(1-(cyclohexylmethyl)-5-methyl-1H-pyrazol-4-yl)-6-(8-((4-fluorobenzo[d]thiazol-2-yl) carbamoyl)-3,4-dihydroisoquinolin-2 (1H)-yl)picolinoyl)sulfamoyl)hexanoate. To a solution of 4-fluoro-1,3-benzothiazol-2-amine (52.5 mg, 312 umol) and 2-(6-(((6-(tert-butoxy)-6-oxohexyl)sulfonyl) carbamoyl)-5-(1-(cyclohexylmethyl)-5-methyl-1H-pyrazol-4-yl) pyridin-2-yl)-1,2,3,4-tetrahydroisoquinoline-8-carboxylic acid (0.13 g, 156 umol) in DMF (2 mL) was added DIEA (60.5 mg, 468 umol) and [dimethylamino (fluoro)methy lene]-dimethyl-ammonium: hexafluorophosphate (61.9 mg, 234 umol). The mixture was then stirred at 60° C., for 16 hrs. On completion, the reaction was diluted with water (20 mL), and extracted with EtOAc (5 mL×3). The combined organic layer was washed with brine (10 mL), dried over Na₂SO₄, filtered and concentrated in vacuo. The crude was purified by reversed phase HPLC (0.1% FA condition) to afford the title compound (60 mg, 43% yield) as a yellow solid. LC-MS (ESI⁺) m/z 858.7 (M+H)⁺.

Step 3 6—(N-(3-(1-(cyclohexylmethyl)-5-methyl-1H-pyrazol-4-yl)-6-(8-((4-fluorobenzo[d]thiazol-2-yl) carbamoyl)-3,4-dihydroisoquinolin-2 (1H)-yl)picolinoyl)sulfamoyl)hexanoic acid. To a solution of tert-butyl 6-[[3-[1-(cyclohexylmethyl)-5-methyl-pyrazol-4-yl]-6-[8-[(4-fluoro-1,3-benzothiazol-2-yl) carbamoyl]-3,4-dihydro-1H-isoquinolin-2-yl]pyridine-2-carbonyl]sulfamoyl]hexanoate (60 mg, 69.9 umol) in DCM (0.5 mL) was added HCl/dioxane (4 M, 1 mL). The mixture was then stirred at 20° C., for 4 hrs. On completion, the reaction was diluted with water (50 mL), then it was extracted with DCM (20 mL×3). The combined organic layer was washed with brine (30 mL), dried over Na₂SO₄, filtered and concentrated in vacuo. The crude was purified by reversed phase HPLC (0.1% FA condition) to afford the title compound (50 mg, 80% yield) as a yellow solid. LC-MS (ESI⁺) m/z 802.6 (M+H)⁺.

### 3-(3,6-dichloro-5-methylpyridazin-4-yl) propanal (Intermediate HM)

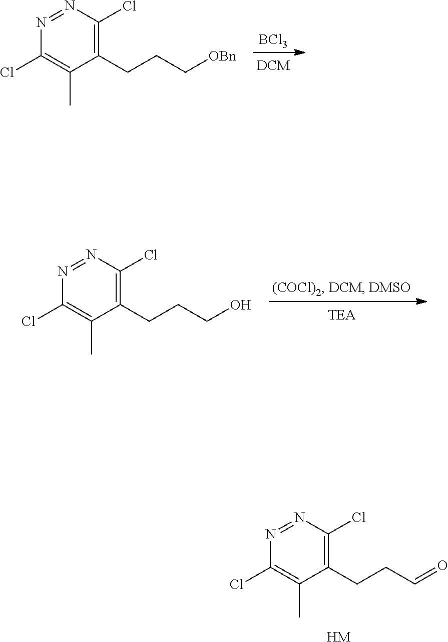

Step 1—3-(3,6-Dichloro-5-methylpyridazin-4-yl) propan-1-ol. To a solution of 4-(3-(benzyloxy)propyl)-3,6-dichloro-5-methylpyridazine (6.00 g, 19.28 mmol, synthesized via Steps 1-3 of Intermediate AP) in DCM (20 mL) was added BCI; (1 M, 193 mL) at 0° C., then the mixture was stirred at 25° C., for 4 h. On completion, the reaction mixture was quenched with NH₄Cl (80 mL) and extracted with EtOAc (100 mL×3). The combined organic layers were washed with brine (20 mL×2), dried over Na₂SO₄, filtered and concentrated under reduced pressure to give a residue. The residue was purified by silica gel column chromatography (Petroleum ether:Ethyl acetate=11:1˜5:1) to give the title compound (3.10 g, 73% yield) as a white solid. ¹H NMR (400 MHZ, DMSO-d₆) δ 4.65 (t, J=5.2 Hz, 1H), 3.50-3.46 (m, 2H), 2.86-2.82 (m, 2H), 2.43 (s, 3H), 1.68-1.61 (m, 2H).

Step 2—3-(3,6-Dichloro-5-methylpyridazin-4-yl) propanal. To an oven-dried flask was added DMSO (1.46 g, 18.7 mmol, 1.46 mL) and DCM (20 mL) and the solution was cooled to −78° C. Then (COCl)₂ (1.49 g, 11.7 mmol, 1.02 mL) was added dropwise and the reaction was allowed to stir for 1 h. Next, 3-(3,6-dichloro-5-methylpyridazin-4-yl) propan-1-ol (1.73 g, 7.80 mmol) in DCM (6 mL) was then added dropwise and the mixture was allowed to stir for 1 h. Then, TEA (4.74 g, 46.8 mmol, 6.52 mL) was added and the reaction was allowed to warm to 0° C., for 1 h. On completion, the reaction was quenched with water (5 mL), then partitioned between saturated NaHCO; (15 mL) and dichloromethane (15 mL). The aqueous phase was extracted with dichloromethane (10 mL), and the combined organic extracts were washed with brine (8 mL), dried over magnesium sulfate and concentrated in vacuo. The residue was purified by column chromatography (SiO₂, Petroleum ether/Ethyl acetate=5:1˜ 1:1) to give the title compound (1.23 g, 72% yield) as a white solid. ¹H NMR (400 MHZ, DMSO-d₆) δ 9.70 (s, 1H), 3.06-2.98 (m, 2H), 2.81-2.73 (m, 2H), 2.43 (s, 3H).

### 1-Isobutyl-5-methyl-4-(4,4,5,5-tetramethyl-1,3,2-dioxaborolan-2-yl)-1H-pyrazole (Intermediate HN)

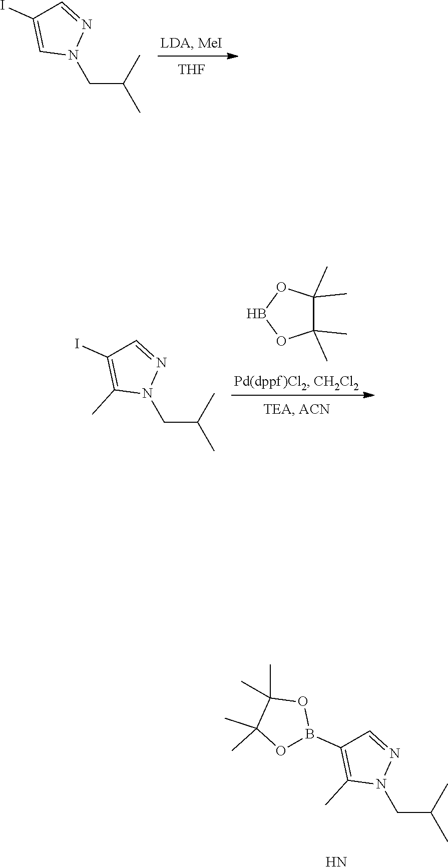

Step 1—4-Iodo-1-isobutyl-5-methyl-1H-pyrazole. To a solution of 4-iodo-1-isobutyl-pyrazole (2 g, 8.00 mmol, CAS #918487-09-9) in THF (20 mL) was added LDA (2 M, 4.80 mL) at −78° C. The mixture was stirred at −78° C., for 1 h, then Mel (1.29 g, 9.12 mmol, 568 uL) was added dropwise slowly. The mixture was then stirred at 25° C., for 12 h. On completion, the reaction mixture was poured into NH₄Cl (sat, aq, 20 mL) and extracted with EtOAc (20 mL×3). The combined organic layers were washed with brine (20 mL×3), dried over sodium sulfate, then filtered and concentrated under reduced pressure to give a residue. The residue was purified by flash silica gel chromatography (ISCOR: 12 g SepaFlash® Silica Flash Column, Eluent of 0˜10% Ethyl acetate/Petroleum ethergradient @ 60 mL/min to give the title compound (2 g, 95% yield) as a yellow solid. ¹H NMR (400 MHZ, DMSO-d₆) δ 7.43 (s, 1H), 3.90 (d, J=7.2 Hz, 2H), 2.25 (s, 3H), 2.06 (quin, J=6.8, 13.6 Hz, 1H), 0.82 (d, J=6.8 Hz, 6H).

Step 2—1-Isobutyl-5-methyl-4-(4,4,5,5-tetramethyl-1,3,2-dioxaborolan-2-yl)-1H-pyrazole. To a solution of 4-iodo-1-isobutyl-5-methyl-pyrazole (1.8 g, 6.82 mmol) and 4,4,5,5-tetramethyl-1,3,2-dioxaborolane (2.62 g, 20.5 mmol) in ACN (18 mL), was added TEA (2.07 g, 20.5 mmol) and Pd (dppf) Cl2·CH₂Cl₂ (557 mg, 682 umol), then the mixture was stirred at 80° C., for 12 h. On completion, the reaction mixture was poured into ice water (20 mL) and extracted with EtOAc (40 mL×3). The combined organic layers were washed with brine (40 mL×3), dried over sodium sulfate, then filtered and concentrated under reduced pressure to give a residue. The residue was purified by flash silica gel chromatography (ISCOR: 20 g SepaFlash® Silica Flash Column, Eluent of 0˜25% Ethyl acetate/Petroleum ethergradient @60 mL/min) to give the title compound (1.3 g, 63% yield) as a yellow solid. ¹H NMR (400 MHz, DMSO-d₆) δ 7.45 (s, 1H), 3.81 (d, J=7.2 Hz, 2H), 2.36 (s, 3H), 2.14-2.03 (m, 1H), 1.24 (s, 12H), 0.82 (d, J=6.4 Hz, 6H).

### 6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinic acid (Intermediate HO)

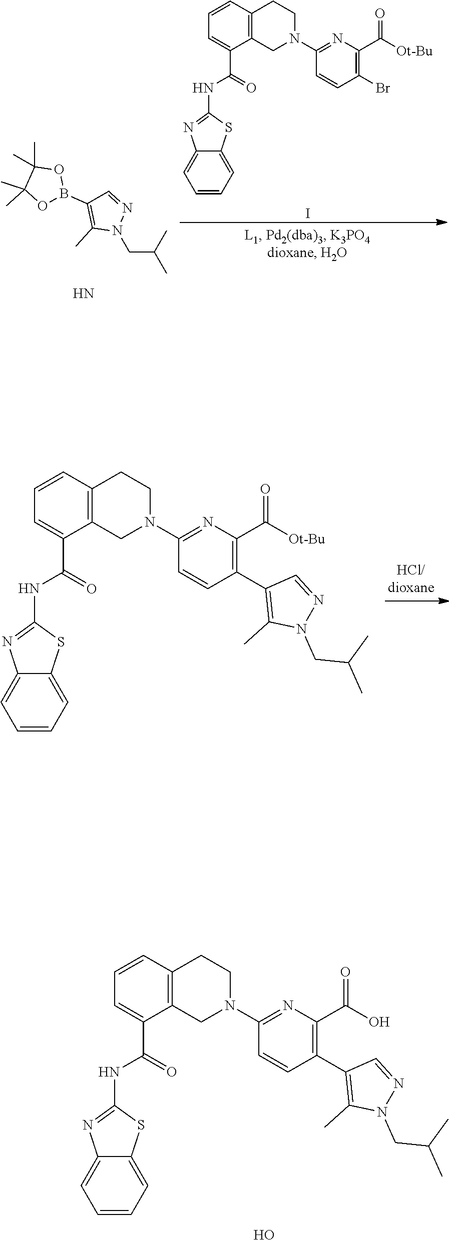

Step 1—Tert-butyl 6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinate. To a solution of 1-isobutyl-5-methyl-4-(4,4,5,5-tetramethyl-1,3,2-dioxaborolan-2-yl) pyrazole (934 mg, 3.54 mmol, Intermediate HN) and tert-butyl 6-(8-(benzo[d]thiazol-2-ylcarbamoyl)-3,4-dihydroisoquinolin-2 (1H)-yl)-3-bromopicolinate (1 g, 1.77 mmol, Intermediate I) in H₂O (10 mL) and dioxane (10 mL), was added K₃PO₄ (1.13 g, 5.31 mmol), (1S,3R,5R,7S)-1,3,5,7-tetramethyl-8-phenyl-2,4,6-trioxa-8-phosphatricyclo[3.3.1.1^{3.7}]decane (207 mg, 707 umol, L1) and Pd₂(dba); (162 mg, 177 umol) at 20° C., under N₂ atmosphere. Then the reaction was stirred at 100° C., for 2 h under N₂ atmosphere. On completion, the reaction mixture was poured into ice water (20 mL) and extracted with EtOAc (20 mL×3). The combined organic layers were washed with brine (20 mL×3), dried over sodium sulfate, filtered and concentrated under reduced pressure to give a residue. The residue was purified by flash silica gel chromatography (ISCOR: 20 g SepaFlash® Silica Flash Column, Eluent of 0˜35% Ethyl acetate/Petroleum ether gradient (@80 mL/min) to give the title compound (600 mg, 55% yield) as a yellow solid. ¹H NMR (400 MHZ, DMSO-d₆) δ 12.85 (s, 1H), 8.02 (d, J=7.6 Hz, 1H), 7.78 (d, J=8.0 Hz, 1H), 7.58 (br d, J=7.2 Hz, 1H), 7.50-7.40 (m, 3H), 7.35 (q, J=7.2 Hz, 2H), 7.21 (s, 1H), 6.93 (d, J=8.8 Hz, 1H), 4.96 (s, 2H), 3.85-3.80 (m, 4H), 3.02 (br t, J=5.6 Hz, 2H), 2.08 (s, 4H), 1.15 (s, 9H), 0.85 (d, J=6.8 Hz, 6H).

Step 2—6-(8-(Benzo[d]thiazol-2-ylcarbamoyl)-3,4-dihydroisoquinolin-2 (1H)-yl)-3-(1-isobutyl-5-methyl-1H-pyrazol-4-yl)picolinic acid. To a solution of tert-butyl 6-(8-(benzo[d]thiazol-2-ylcarbamoyl)-3,4-dihydroisoquinolin-2 (1H)-yl)-3-(1-isobutyl-5-methyl-1H-pyrazol-4-yl)picolinate (600 mg, 964 umol) in HCl/dioxane (4 M, 20.00 mL), the mixture was stirred at 25° C., for 2 h. On completion, the reaction mixture was concentrated under reduced pressure to give the title compound (600 mg, HCl) as a yellow solid. ¹H NMR (400 MHZ, DMSO-d₆) δ 13.19-12.30 (m, 1H), 8.04 (d, J=7.6 Hz, 1H), 7.79 (d, J=8.0 Hz, 1H), 7.62 (d, J=7.6 Hz, 1H), 7.53-7.49 (m, 1H), 7.48-7.41 (m, 2H), 7.39-7.32 (m, 3H), 6.97 (d, J=8.8 Hz, 1H), 4.96 (s, 2H), 3.91-3.83 (m, 4H), 3.01 (br t, J=5.6 Hz, 2H), 2.11 (s, 3H), 2.09-2.05 (m, 1H), 0.84 (d, J=6.4 Hz, 6H).

### Methyl-2-(5-bromo-6-(tert-butoxycarbonyl) pyridin-2-yl)-1,2,3,4-tetrahydroisoquinoline-8-carboxylate (Intermediate HP)

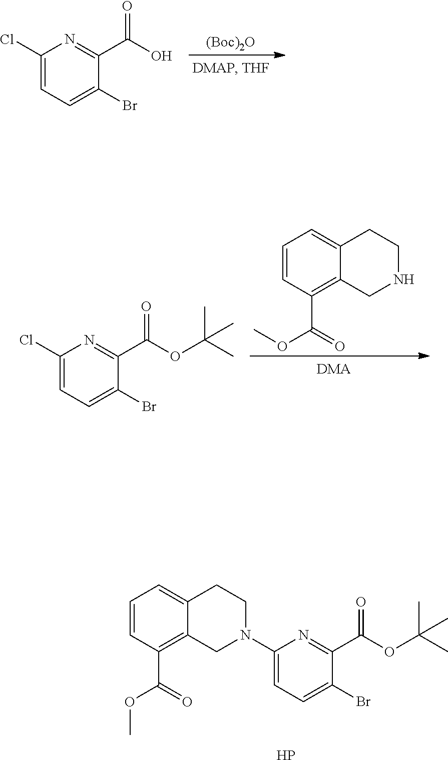

Step 1—Tert-butyl 3-bromo-6-chloropicolinate. To a solution of 3-bromo-6-chloropicolinic acid (250 g, 1.06 mol, CAS #929000-66-8), tert-butoxycarbonyl tert-butyl carbonate (461 g, 2.11 mol) in THF (2000 mL) was added DMAP (12.9 g, 105 mmol). The mixture was stirred at 40° C., for 3 hrs. The reaction mixture was quenched by addition NaHCO; (500 mL) at 25° C., and then diluted with H₂O (600×3) and extracted with EtOAc (1200 mL×3). The combined organic layers were dried over Na₂SO₄ filtered and concentrated under reduced pressure to give a residue. The residue was purified by column chromatography (SiO₂, Petroleum ether/Ethyl acetate=20/1 to 5/1) to give the title compound (310 g) as a white solid. ¹H NMR (400 MHZ, DMSO-d₆) δ=8.26 (d, J=8.4 Hz, 1H), 7.63 (d, J=8.4 Hz, 1H), 1.56 (s, 9H).

Step 2 Methyl-2-(5-bromo-6-(tert-butoxycarbonyl) pyridin-2-yl)-1,2,3,4-tetrahydroisoquinoline-8-carboxylate. To a solution of 3-bromo-6-chloropicolinate (20 g, 68.3 mmol), methyl 1,2,3,4-tetrahydroisoquinoline-8-carboxylate (17.1 g, 75.2 mmol, CAS #1028330-54-2) in DMA (300 mL) was added DIEA (44.1 g, 341 mmol). The mixture was stirred at 120° C., for 18 hrs. On completion, the reaction mixture was diluted with H₂O (500 mL) and extracted with EtOAc (300 mL×4). The combined organic layers were washed with brine (300 mL×3), dried over Na₂SO₄ filtered and concentrated under reduced pressure to give a residue. The residue was purified by column chromatography (SiO₂, Petroleum ether/Ethyl acetate=100/1 to 10/1) to give the title compound (13 g, 39% yield) as a yellow solid. LC-MS (ESI⁺) m/z 447.2 (M+H)⁺.

### Benzyl 2-(methyl (3-sulfamoylpropyl)amino)acetate (Intermediate HQ)

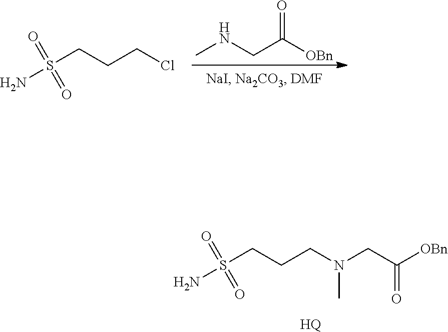

To a solution of 3-chloropropane-1-sulfonamide (500 mg, 3.17 mmol, CAS #35578-28-0) and benzyl 2-(methylamino)acetate (799 mg, 3.17 mmol, HCl, CAS #40298-32-6) in DMF (10 mL) was added NaI (476 mg, 3.17 mmol) and Na₂CO; (1.34 g, 12.7 mmol). Then the mixture was stirred at 70° C., for 5 h. On completion, the mixture was filtered. The mixture was purified by reversed-phase HPLC (0.1% NH₃·H₂O condition) to give the title compound (410 mg, 35% yield) as a white solid. ¹HNMR (400 MHZ, CDCl₃-d) δ=7.41-7.33 (m, 5H), 5.43 (br s, 2H), 5.16 (s, 2H), 3.30 (s, 2H), 3.23 (t, J=7.2 Hz, 2H), 2.63 (t, J=6.4 Hz, 2H), 2.37 (s, 3H), 2.05 (quin, J=6.4 Hz, 2H).

### 2-((3-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl) propyl)(methyl)amino)acetic acid (Intermediate HR)

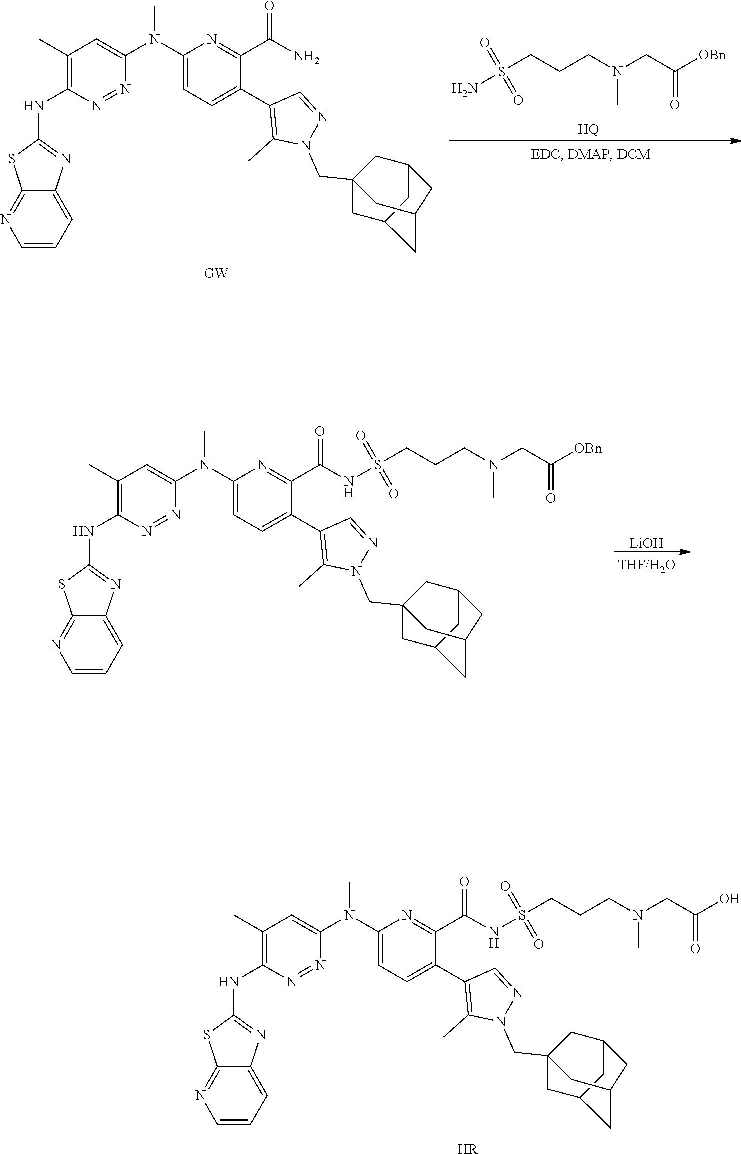

Step 1—Benzyl 2-((3-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl) propyl)(methyl)amino)acetate. To a solution of benzyl 2-(methyl (3-sulfamoylpropyl)amino)acetate (242 mg, 643 umol, Intermediate HQ) and 3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinic acid (200 mg, 322 umol, Intermediate GW) in DCM (8 mL) was added DMAP (78.6 mg, 643 umol) and 4A molecular sieves (200 mg, 322 umol). Then EDC (99.9 mg, 643 umol) was added in batches and the mixture was stirred at 25° C., for 2 h. On completion, the reaction mixture was poured into water (4 mL), and then extracted with DCM (4 mL×3). The combined organic layers were washed with brine (4 mL×3), dried over Na₂SO₄, filtered and concentrated under reduced pressure to give a residue. The residue was purified by column chromatography (SiO₂, Dichloromethane:Methanol=20/1-10/1), then re-purified by reversed-phase HPLC (0.1% FA condition) to give the title compound (60.0 mg, 21% yield) as a yellow solid. LC-MS (ESI⁺) m/z 904.3 (M+H)⁺.

Step 2—2-((3-(N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl) propyl)(methyl)amino)acetic acid. To a solution of benzyl 2-((3-(N-(3-(1-((1s, 3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl) propyl)(methyl)amino)acetate (60.0 mg, 66.4 umol) in THF (2 mL) and H₂O (1 mL) was added LiOH (7.95 mg, 332 umol), then the mixture was stirred at 20-40° C., for 5 h. On completion, the mixture was diluted with DCM (1 ml) and water (1 ml) and separated. Then the aqueous phase was adjusted pH=10 to 6 with HCl solution, then the mixture was extracted with DCM:THF=4:1 (2 ml×3). The organic phase was and brine (2 ml×3), dried over Na₂SO₄ and concentrated to give the title compound (50 mg) as a yellow solid. ¹H NMR (400 MHZ, DMSO-d₆) δ=8.39-8.33 (m, 1H), 7.95-7.90 (m, 1H), 7.83 (s, 1H), 7.73-7.69 (m, 1H), 7.43 (dd, J=4.8, 8.4 Hz, 1H), 7.38 (s, 1H), 7.36-7.30 (m, 1H), 3.76-3.72 (m, 2H), 3.66 (s, 3H), 3.62-3.58 (m, 2H), 2.40 (br s, 3H), 2.20 (s, 3H), 1.94 (br s, 4H), 1.87-1.79 (m, 3H), 1.78-1.73 (m, 2H), 1.70-1.58 (m, 6H), 1.55 (br s, 7H), 1.37-1.33 (m, 2H), 1.26-1.20 (m, 2H).

### Benzyl 2-(4-(sulfamoylmethyl)piperidin-1-yl)acetate (Intermediate HS)

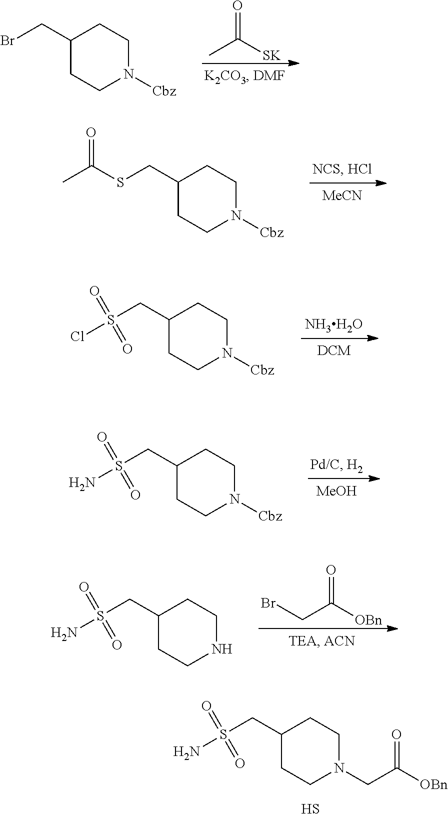

Step 1—Benzyl 4-((acetylthio) methyl)piperidine-1-carboxylate. To a solution of benzyl 4-(bromomethyl)piperidine-1-carboxylate (7.00 g, 22.4 mmol, CAS #159275-17-9) and K₂CO₃ (6.20 g, 44.8 mmol) in DMF (70 mL) was added potassium ethanethioate (3.33 g, 29.2 mmol, CAS #10387-40-3) dropwise at 0° C., under N₂, then the mixture was stirred at 25° C., for 2 h. On completion, the reaction mixture was poured into water (70 mL), and then extracted with EtOAc (100 mL×3). The combined organic layers were washed with brine (100 mL×3), dried over sodium sulfate, filtered and concentrated under reduced pressure to give a residue. The residue was purified by column chromatography (SiO₂, Petroleum ether/Ethyl acetate=5/1 to 0:1) to give the title compound (6.00 g, 87% yield) as a white oil. LC-MS (ESI⁺) m z 308.0 (M+H); ¹H NMR (400 MHZ, DMSO-d₆) δ=7.42-7.27 (m, 5H), 5.06 (s, 2H), 4.02-3.94 (m, 2H), 2.84-2.70 (m, 4H), 2.33 (s, 3H), 1.70-1.55 (m, 3H), 1.12-0.99 (m, 2H).

Step 2—Benzyl 4-((chlorosulfonyl)methyl)piperidine-1-carboxylate. To a solution of benzyl 4-((acetylthio) methyl)piperidine-1-carboxylate (5.00 g, 16.3 mmol) in MeCN (50 mL) was added HCl (2 M, 12 mL) and NCS (8.69 g, 65.1 mmol, CAS #128-09-6). The mixture was stirred at 0-25° C., for 0.5 h. On completion, the reaction mixture was poured into water (50 mL), and then extracted with EtOAc (100 mL×3). The combined organic layers were washed with brine (30 mL×3), dried over sodium sulfate, filtered and concentrated under reduced pressure to give a title compound (5.00 g) as a white oil.

Step 3—Benzyl 4-(sulfamoylmethyl)piperidine-1-carboxylate. To a solution of benzyl 4-((chlorosulfonyl)methyl)piperidine-1-carboxylate (5.00 g, 15.1 mmol) in DCM (30 mL) was added NH₃·H₂O (54.6 g, 389 mmol, 60 mL). The mixture was then stirred at 0-25° C., for 1 h. On completion, the reaction mixture was washed with water (30 mL×3), brine (30 mL×3), dried over sodium sulfate, filtered and concentrated under reduced pressure to give a residue. The residue was purified by column chromatography (SiO₂, Petroleum ether/Ethyl acetate=8/1 to 0:1) to give the title compound (4.00 g, 85% yield) as a white oil. LC-MS (ESI) m/z 313.3 (M+H); ¹H NMR (400 MHZ, DMSO-d₆) δ=7.39-7.31 (m, 4H), 6.83 (s, 2H), 5.06 (s, 2H), 4.00-3.93 (m, 2H), 2.99-2.76 (m, 4H), 2.56 (s, 1H), 2.12-1.96 (m, 2H), 1.91-1.78 (m, 2H), 1.26-1.12 (m, 3H).

Step 4—Piperidin-4-ylmethanesulfonamide. To a solution of benzyl 4-(sulfamoylmethyl)piperidine-1-carboxylate (4.00 g, 12.8 mmol) in MeOH (30 mL) was added Pd/C (1.00 g. 10 wt %). The mixture was then stirred at 25° C., for 12 h under H₂. On completion, the reaction mixture was filtered and concentrated under reduced pressure to give the title compound (2.60 g) as white oil. 1H NMR (400 MHz, DMSO-d₆) ¿=7.00-6.56 (m, 2H), 2.91-2.86 (m, 4H), 2.48-2.42 (m, 2H), 1.96-1.72 (m, 4H), 1.20-1.10 (m, 2H).

Step 5—Benzyl 2-(4-(sulfamoylmethyl)piperidin-1-yl)acetate. To a solution of piperidin-4-ylmethanesulfonamide (2.60 g, 14.6 mmol), benzyl 2-bromoacetate (5.01 g, 21.9 mmol, 3.43 mL, CAS #5437-45-6) in ACN (30 mL) was added TEA (2.95 g, 29.2 mmol, 4.06 mL). The mixture was stirred at 25° C., for 12 h. On completion, the reaction mixture was poured into water (30 mL), and then extracted with EtOAc (50 mL×3). The combined organic layers were washed with brine (20 mL×3), dried over sodium sulfate, filtered and concentrated under reduced pressure to give a residue. The residue was purified by column chromatography (SiO₂, Petroleum ether/Ethyl acetate=3/1 to 0:1) to give title compound (2.70 g, 57% yield) as a white oil. LC-MS (ESI) m/z 327.0 (M+H); ¹H NMR (400 MHZ, DMSO-d₆) δ=7.46-7.24 (m, 5H), 6.79 (s, 2H), 5.11 (s, 2H), 3.25 (s, 2H), 2.91 (d, J=6.0 Hz, 2H), 2.79 (br d, J=11.6 Hz, 2H), 2.18 (br t, J=10.5 Hz, 2H), 1.80 (br d, J=10.6 Hz, 3H), 1.38-1.21 (m, 2H).

### 2-(4-((N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)methyl)piperidin-1-yl)acetic acid (Intermediate HT)

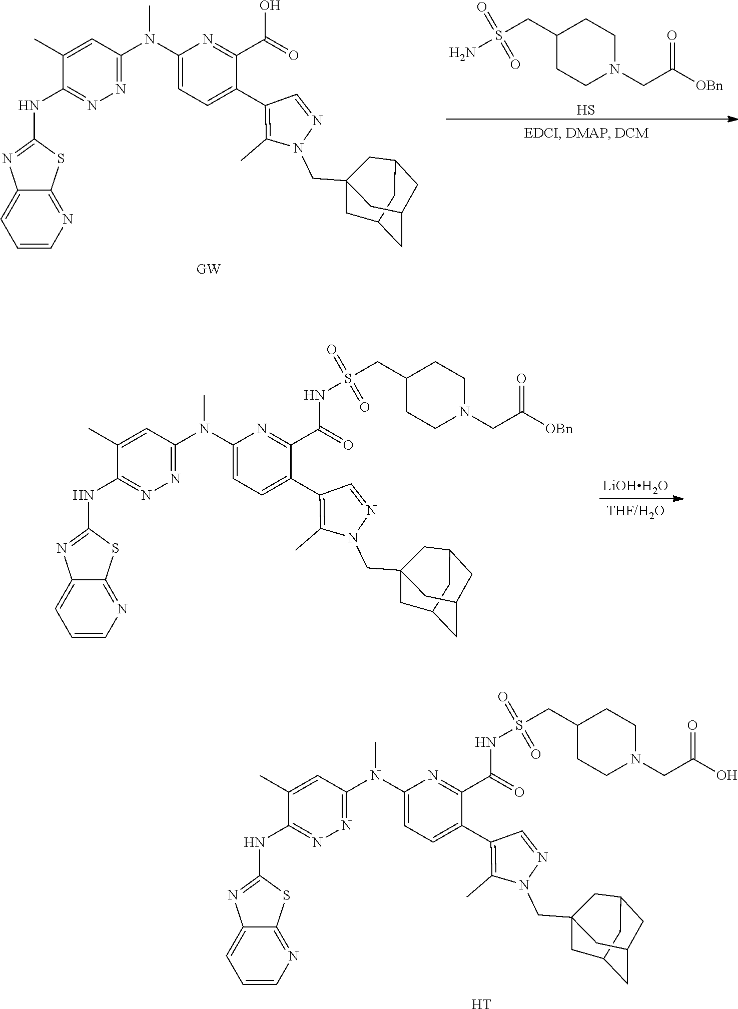

Step 1—Benzyl 2-(4-((N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)methyl)piperidin-1-yl)acetate. To a solution of 3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinic acid (400 mg, 547 umol, HCl salt, Intermediate GW), benzyl 2-(4-(sulfamoylmethyl)piperidin-1-yl)acetate (536 mg, 1.64 mmol, Intermediate HS) in DCM (5 mL) was added EDCI (210 mg, 1.09 mmol) and DMAP (267 mg, 2.19 mmol). The mixture was then stirred at 25° C., for 12 h. On completion, the reaction was poured into water (5 mL) and extracted with DCM (10 mL×3). The combined organic phase is washed with brine (5 mL×2), dried over sodium sulfate, then filtered to get the filtrate and concentrated to give a residue. The residue was purified by prep-HPLC (column: Phenomenex Luna C18 150*25 mm*10 um; mobile phase: [water (FA)-ACN]; B %: 35%-65%, 8 min) to give title compound (130 mg, 26% yield) as a yellow solid. LC-MS (ESI⁺) m/z 930.5 (M+H)⁺.

Step 2—2-(4-((N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)methyl)piperidin-1-yl)acetic acid. To a solution of benzyl 2-(4-((N-(3-(1-((1s,3s)-adamantan-1-ylmethyl)-5-methyl-1H-pyrazol-4-yl)-6-(methyl (5-methyl-6-(thiazolo[5,4-b]pyridin-2-ylamino)pyridazin-3-yl)amino)picolinoyl)sulfamoyl)methyl)piperidin-1-yl)acetate (110 mg, 118 umol) in THF (0.5 mL) and H₂O (0.5 mL) was added LiOH·H₂O (24.8 mg, 591 umol). The mixture was stirred at 25° C., for 2 h. On completion, the pH of the reaction mixture was adjusted to 6˜5 by addition of 2 M HCl, then the mixture was extracted with DCM (2 mL×5). The organic phase was separated, dried over Na₂SO₄ filtered and concentrated under reduced pressure to give title compound (150 mg) as a yellow solid. LC-MS (ESI⁺) m/z 840.5 (M+H)⁺.

### 6-(N-(3-(1-(cyclohexylmethyl)-5-methyl-1H-pyrazol-4-yl)-6-(8-((5-methylthiazol-2-yl) carbamoyl)-3,4-dihydroisoquinolin-2 (1H)-yl)picolinoyl)sulfamoyl)hexanoic acid (Intermediate HU)

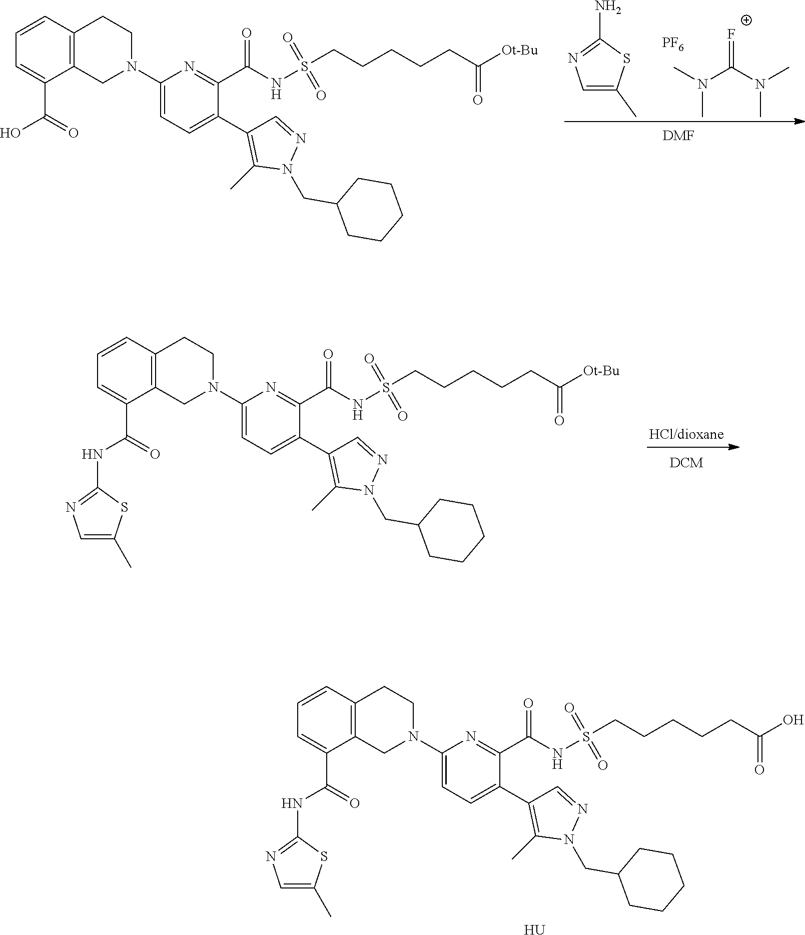

Step 1—Tert-butyl 6-(N-(3-(1-(cyclohexylmethyl)-5-methyl-1H-pyrazol-4-yl)-6-(8-((5-methylthiazol-2-yl) carbamoyl)-3,4-dihydroisoquinolin-2 (1H)-yl)picolinoyl)sulfamoyl)hexanoate. To a solution of 2-[6-[(6-tert-butoxy-6-oxo-hexyl)sulfonylcarbamoyl]-5-[1-(cyclohexylmethyl)-5-methyl-pyrazol-4-yl]-2-pyridyl]-3,4-dihydro-1H-isoquinoline-8-carboxylic acid (0.12 g, 170 umol, synthesized via Step 1 of Intermediate HL) and 5-methylthiazol-2-amine (38.7 mg, 339 umol) in DMF (2 mL) was added [dimethylamino (fluoro) methylene]-dimethyl-ammonium: hexafluorophosphate (90.0 mg, 339 umol) and DIEA (110 mg, 848 umol). The mixture was then stirred at 60° C., for 16 hrs. On completion, the reaction was diluted with water (20 mL), and extracted with EA (10 mL×3). The combined organic layer was washed with sat. NH₄Cl (20 mL) and brine (20 mL), dried over Na₂SO₄, filtered and concentrated in vacuo. The crude was purified by reversed phase HPLC (0.1% FA condition) to afford the title compound (100 mg, 66% yield) as a yellow solid. LC-MS (ESI⁺) m/z 804.7 (M+H)⁺.

Step 2—6-(N-(3-(1-(cyclohexylmethyl)-5-methyl-1H-pyrazol-4-yl)-6-(8-((5-methylthiazol-2-yl) carbamoyl)-3,4-dihydroisoquinolin-2 (1H)-yl)picolinoyl)sulfamoyl)hexanoic acid. To a solution of tert-butyl 6-[[3-[1-(cyclohexylmethyl)-5-methyl-pyrazol-4-yl]-6-[8-[(5-methylthiazol-2-yl) carbamoyl]-3,4-butyl dihydro-1H-isoquinolin-2-yl]pyridine-2-carbonyl]sulfamoyl]hexanoate (0.1 g, 124.37 umol) in DCM (1 mL) was added HCl/dioxane (4 M, 1.00 mL). The mixture was stirred at 20° C., for 16 hrs. On completion, the reaction was filtered and the filter cake was washed with DCM (2 mL×2) to afford the title compound (70 mg) as a yellow solid. LC-MS (ESI⁺) m/z 748.6 (M+H)+.

### Oxazol-5-ylmethanamine (CAS #847644-09-1) (Intermediate HV)

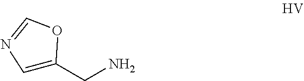

### 6-(N-(3-(1-(cyclohexylmethyl)-5-methyl-1H-pyrazol-4-yl)-6-(8-((4-methylthiazol-2-yl) carbamoyl)-3,4-dihydroisoquinolin-2 (1H)-yl)picolinoyl)sulfamoyl)hexanoic acid (Intermediate HW)

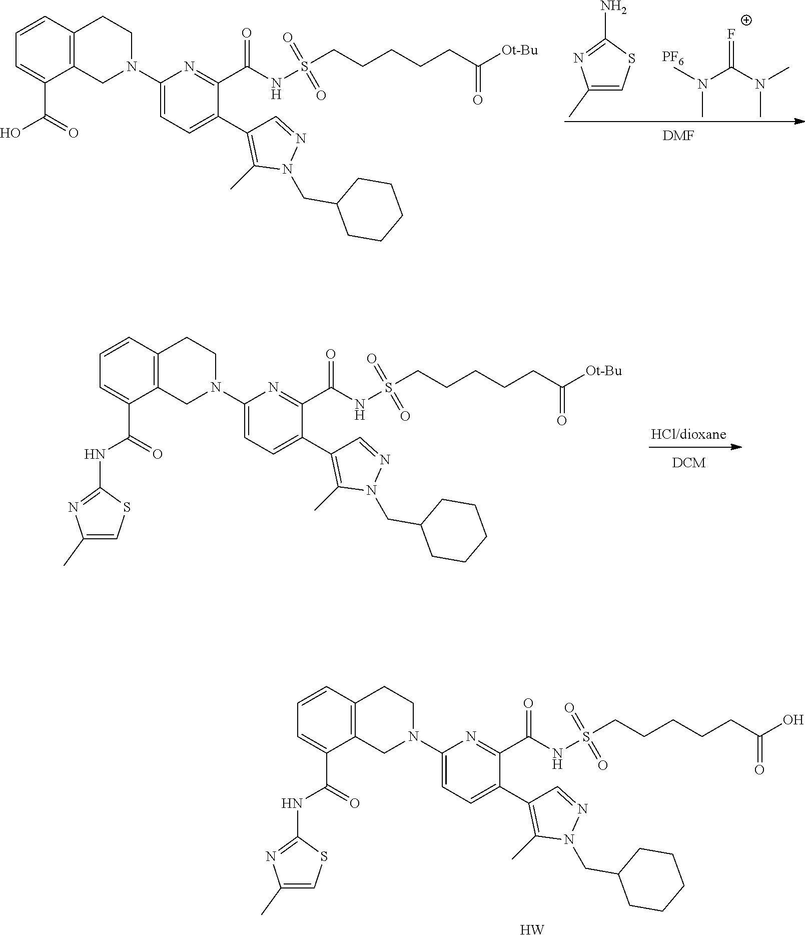

Step 1—Tert-butyl 6-(N-(3-(1-(cyclohexylmethyl)-5-methyl-1H-pyrazol-4-yl)-6-(8-((4-methylthiazol-2-yl) carbamoyl)-3,4-dihydroisoquinolin-2 (1H)-yl)picolinoyl)sulfamoyl)hexanoate. To a solution of 2-[6-[(6-tert-butoxy-6-oxo-hexyl)sulfonylcarbamoyl]-5-[1-(cyclohexylmethyl)-5-methyl-pyrazol-4-yl]-2-pyridyl]-3,4-dihydro-1H-isoquinoline-8-carboxylic acid (0.12 g, 170 umol, synthesized via Step 1 of Intermediate HL) and 4-methylthiazol-2-amine (38.7 mg, 339 umol) in DMF (2 mL) was added [dimethylamino (fluoro) methylene]-dimethyl-ammonium hexafluorophosphate (89.6 mg, 339 umol) and DIEA (110 mg, 848 umol). The mixture was stirred at 60° C., for 16 hrs. On completion, the reaction was diluted with water (20 mL), and extracted with EA (10 mL×3). The organic layer was washed with sat. NH₄Cl (20 mL) and brine (20 mL), dried over Na₂SO₄, filtered and concentrated in vacuo. The crude was purified by reversed phase HPLC: (0.1% FA condition) to afford the title compound (0.12 g, 79% yield) as a yellow solid. LC-MS (ESI⁺) m/z 804.7 (M+H)⁺.

Step 2—6-(N-(3-(1-(cyclohexylmethyl)-5-methyl-1H-pyrazol-4-yl)-6-(8-((4-methylthiazol-2-yl) carbamoyl)-3,4-dihydroisoquinolin-2 (1H)-yl)picolinoyl)sulfamoyl)hexanoic acid. To a solution of tert-butyl 6-[[3-[1-(cyclohexylmethyl)-5-methyl-pyrazol-4-yl]-6-[8-[(4-methylthiazol-2-yl) carbamoyl]-3,4-dihydro-1H-isoquinolin-2-yl]pyridine-2-carbonyl]sulfamoyl]hexanoate (0.12 g, 149 umol) in DCM (1 mL) was added HCl/dioxane (4 M, 1.33 mL). The mixture was stirred at 20° C., for 4 hrs. On completion, the reaction was filtered and the filter cake was washed with DCM (2 mL×2) to afford the title compound (0.1 g) as a yellow solid. LC-MS (ESI⁺) m/z 748.6 (M+H)⁺.

### Tert-butyl 2-(2-(2-sulfamoylethoxy)ethoxy)acetate (Intermediate HX)

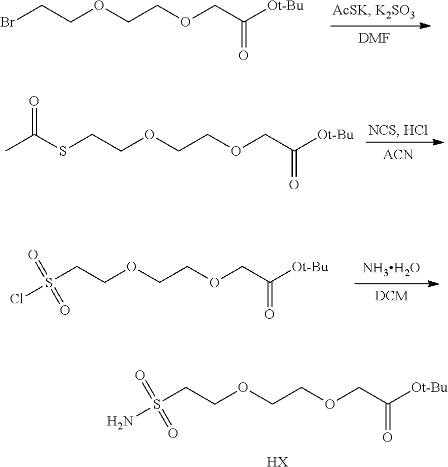

Step 1—Tert-butyl 2-(2-(2-(acetylthio) ethoxy) ethoxy)acetate. A mixture of tert-butyl 2-(2-(2-bromoethoxy) ethoxy)acetate (2.50 g, 8.83 mmol, CAS #1807518-63-3) and K₂CO; (2.44 g, 17.7 mmol) in DMF (25 mL) was added potassium ethanethioate (1.31 g, 11.5 mmol) dropwise at 0° C., under N₂, then the mixture was stirred at 25° C., for 1.5 h. On completion, the reaction mixture was poured into water (20 mL), and then extracted with EtOAc (30 mL×3). The combined organic layers were washed with brine (30 mL×3), dried over Na₂SO₄, filtered and concentrated under reduced pressure to give the title compound (2.50 g) as a white solid. ¹H NMR (400 MHZ, CDCl₃-d) δ=4.03 (s, 2H), 3.73-3.65 (m, 4H), 3.61 (t, J=6.4 Hz, 2H), 3.10 (t, J=6.4 Hz, 2H), 2.34 (s, 3H), 1.48 (s, 9H).

Step 2—Tert-butyl 2-(2-(2-(chlorosulfonyl)ethoxy) ethoxy)acetate. To a solution of tert-butyl 2-(2-(2-(acetylthio) ethoxy) ethoxy)acetate (500 mg, 1.80 mmol) in ACN (5 mL) was added HCl (2 M, 1.26 mL) and NCS (959 mg, 7.18 mmol, CAS #1222468-90-7) at 0° C. The mixture was then stirred at 20° C. for 0.5 h. On completion, the reaction mixture was poured into water (10 mL), and then extracted with EtOAc (10 mL×3). The combined organic layers were washed with brine (10 mL×3), dried over Na₂SO₄, filtered and concentrated under reduced pressure to give the title compound (540 mg) as a white solid.

Step 3—Tert-butyl 2-(2-(2-sulfamoylethoxy) ethoxy)acetate. A solution of tert-butyl 2-(2-(2-(chlorosulfonyl)ethoxy) ethoxy)acetate (2.10 g, 6.94 mmol) in DCM (20 mL) was added NH₃·H₂O (9.72 g, 69.4 mmol, 25% solution) at 0° C., then the mixture was stirred at 25° C., for 1 h. On completion, the reaction mixture was washed with NaHCO₃ (10 ml×3), washed with brine (10 mL×3), dried over Na₂SO₄, filtered and concentrated under reduced pressure to give a residue. The residue was purified by column chromatography (SiO₂, Petroleum ether/Ethyl acetate=3/1-1/1) to give the title compound (915 mg, 47% yield). ¹H NMR (400 MHZ, CDCl₃-d) ô=5.23 (br s, 2H), 4.02-3.93 (m, 4H), 3.74-3.66 (m, 4H), 3.40-3.34 (m, 2H), 1.50-1.43 (m, 9H).

### 2-[2-[2-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]ethoxy]ethoxy]acetic acid (Intermediate HY)

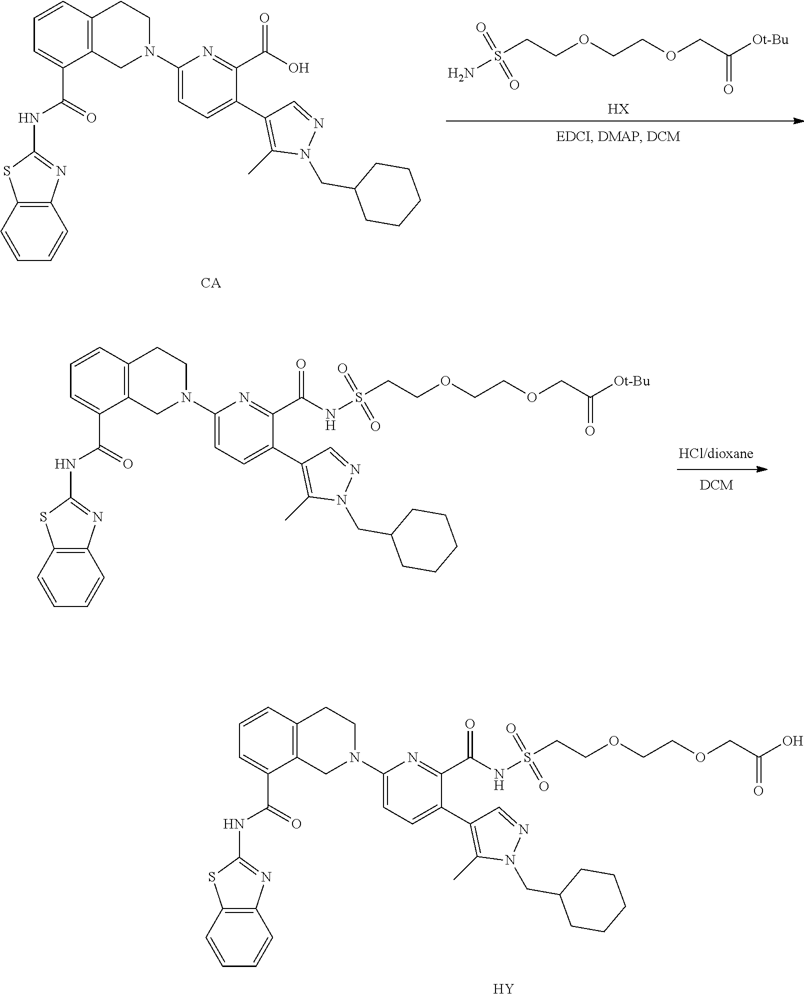

Step 1—Tert-butyl 2-[2-[2-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]ethoxy]ethoxy]acetate. To a solution of tert-butyl 2-[2-(2-sulfamoylethoxy) ethoxy]acetate (841 mg, 2.97 mmol, Intermediate HX) and 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid (400 mg, 659 umol, Intermediate CA) in DCM (4 mL) was added DMAP (322 mg, 2.64 mmol). Then EDCI (253 mg, 1.32 mmol) was added in batches and the mixture was stirred at 25° C., for 2 h. On completion, the reaction mixture was poured into water (2 mL), and then extracted with DCM (2 mL×3). The combined organic layers were washed with brine (2 mL×3), dried over Na₂SO₄, filtered and concentrated under reduced pressure to give a residue. The residue was purified by column chromatography (SiO₂, Dichloromethane:Methanol=20/1-10/1) to give the title compound (139 mg, 24% yield) as a yellow solid. ¹H NMR (400 MHZ, DMSO-d₆) δ=12.90-12.81 (m, 1H), 11.78-11.69 (m, 1H), 8.07-7.99 (m, 1H), 7.82-7.74 (m, 1H), 7.65-7.59 (m, 1H), 7.55-7.51 (m, 1H), 7.49-7.42 (m, 2H), 7.39-7.33 (m, 2H), 7.27 (s, 1H), 7.01 (br d, J=9.6 Hz, 1H), 5.00 (s, 2H), 3.94 (br t, J=6.4 Hz, 2H), 3.85 (d, J=7.2 Hz, 2H), 3.71 (br t, J=6.0 Hz, 2H), 3.60-3.53 (m, 4H), 3.51-3.47 (m, 4H), 3.03 (br t, J=5.6 Hz, 2H), 2.10 (s, 3H), 1.84-1.74 (m, 1H), 1.67-1.59 (m, 3H), 1.53 (br d, J=12.0 Hz, 2H), 1.39 (s, 9H), 1.17-1.12 (m, 3H), 1.01-0.92 (m, 2H).

Step 2—2-[2-[2-[[6-[8-(1,3-Benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]ethoxy]ethoxy]acetic acid. A solution of tert-butyl 2-[2-[2-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]ethoxy]ethoxy]acetate (139 mg, 159 umol) in DCM (1.5 mL) and HCl/dioxane (1.5 mL) was stirred at 25° C., for 2 h. On completion, the mixture was concentrated under reduced pressure to give the title compound (125 mg) as a yellow solid. LC-MS (ESI⁺) m/z 830.5 (M+14)⁺.

### (2S,4R)-1-((S)-2-amino-3,3-dimethylbutanoyl)-N-((S)-1-(4-cyanophenyl)ethyl)-4-hydroxypyrrolidine-2-carboxamide (Intermediate HZ)

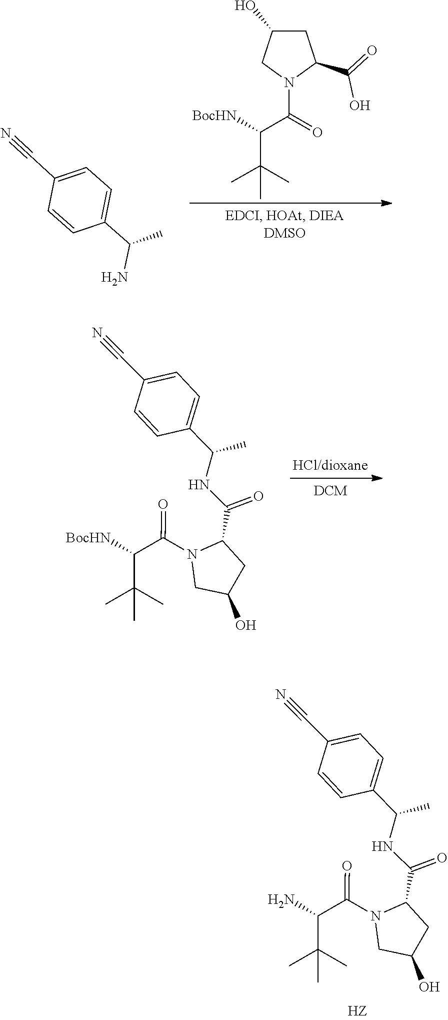

Step 1—Tert-butyl ((S)-1-((2S,4R)-2-(((S)-1-(4-cyanophenyl)ethyl)carbamoyl)-4-hydroxypyrrolidin-1-yl)-3,3-dimethyl-1-oxobutan-2-yl) carbamate. To a solution of(S)-4-(1-aminoethyl)benzonitrile (200 mg, 1.37 mmol, CAS #36244-70-9) and (2S,4R)-1-((S)-2-((tert-butoxycarbonyl)amino)-3,3-dimethylbutanoyl)-4-hydroxypyrrolidine-2-carboxylic acid (471 mg, 1.37 mmol, CAS #630421-46-4) in DMSO (4 mL) was added HOAt (409 mg, 3.01 mmol, 421 uL) and DIEA (884 mg, 6.84 mmol, 1.19 mL). Then EDCI (576 mg, 3.01 mmol) was added and the mixture was stirred at 25° C., for 1 h. On completion, the reaction mixture was quenched with H₂O (20 mL) at 20° C., and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (600 mg, 93% yield) as white solid. LC-MS (ESI⁺) m/z 473.3 (M+H)⁺.

Step 2—(2S,4R)-1-((S)-2-amino-3,3-dimethylbutanoyl)-N-((S)-1-(4-cyanophenyl)ethyl)-4-hydroxypyrrolidine-2-carboxamide. A solution of tert-butyl ((S)-1-((2S,4R)-2-(((S)-1-(4-cyanophenyl)ethyl)carbamoyl)-4-hydroxypyrrolidin-1-yl)-3,3-dimethyl-1-oxobutan-2-yl) carbamate (200 mg, 423 umol) in DCM (2 mL) was added HCl/dioxane (4 M, 4 mL), then the mixture was stirred at 25° C. for 0.5 h. On completion, the mixture was concentrated in vacuo to give the title compound (150 mg, HCl) as white solid. LC-MS (ESI⁺) m/z 373.5 (M+H)⁺.

### 6-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)hexanoic acid (Intermediate IA)

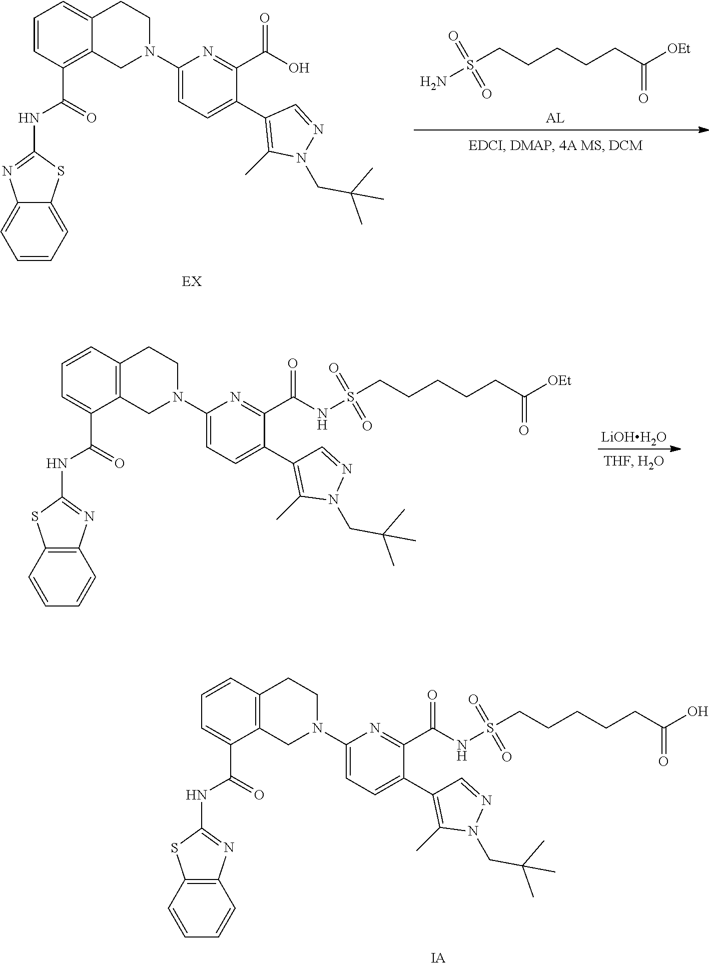

Step 1—Ethyl 6-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)hexanoate. To a solution of 6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinic acid (800.0 mg, 1.38 mmol, Intermediate EZ) and ethyl 6-sulfamoylhexanoate (922 mg, 4.13 mmol, Intermediate AL) in DCM (8 mL) was added EDCI (1.06 g, 5.51 mmol), DMAP (1.35 g, 11.0 mmol) and 4A molecular sieves (15 mg, 1.38 mmol) at 20° C. Then the reaction was stirred at 40° C., for 17 h. On completion, the mixture was quenched with H₂O (8 mL) and extracted with DCM (8 mL×3). The combined organic layers was washed with brine (8 mL×3) and dried over Na₂SO₄ and concentrated in vacuo. The residue was purified by column chromatography on silica gel (eluted with petroleum ether:ethyl acetate=5:1 to 0:1, Dichloromethane:Methanol=10:1) the repurified by re-HPLC (0.1% FA condition, 5%-60%, 20 min) to give the title compound (113 mg, 10% yield) was a yellow solid. LC-MS (ESI⁺) m/z 786.3 (M+1)⁺.

Step 2—6-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)hexanoic acid. To a solution of ethyl 6-(N-(6-(8-(benzo[d]thiazol-2-ylcarbamoyl)-3,4-dihydroisoquinolin-2 (1H)-yl)-3-(5-methyl-1-neopentyl-1H-pyrazol-4-yl)picolinoyl)sulfamoyl)hexanoate (37.0 mg, 47.1 umol) in H₂O (0.5 mL) and THF (0.5 mL) was added LiOH·H₂O (9.88 mg, 235.0 umol) at 20° C. Then the reaction was stirred at 20° C., for 2 h. On completion, the reaction mixture was filtered and extracted with DCM (5 mL×2). The aqueous phase pH was adjusted to 4˜5 with 2M HCl and H₂O (3 mL) was added and the mixture was extracted with DCM (5 mL×3). The combined organic layers was washed with brine (2 mL×3), dried over Na₂SO₄ and concentrated in vacuo to give the title compound (25.0 mg, 59% yield) as a yellow solid. LC-MS (ESI⁺) m z 758.5 (M+1)⁺.

### Tert-butyl 3-bromo-6-(8-(thiazolo[5,4-b]pyridin-2-ylcarbamoyl)-3,4-dihydroisoquinolin-2 (1H)-yl)picolinate (Intermediate IB)

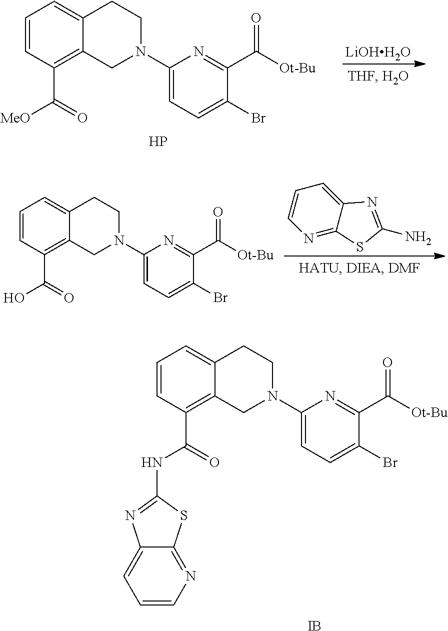

Step 1—2-(5-Bromo-6-(tert-butoxycarbonyl) pyridin-2-yl)-1,2,3,4-tetrahydroisoquinoline-8-carboxylic acid. To a solution of methyl 2-(5-bromo-6-(tert-butoxycarbonyl) pyridin-2-yl)-1,2,3,4-tetrahydroisoquinoline-8-carboxylate (10.0 g, 22.4 mmol, Intermediate HP) in H₂O (30 mL) and THF (60 mL) was added LiOH·H₂O (4.69 g, 112 mmol). The mixture was stirred at 40° C., for 12 hr. On completion, the reaction mixture was filtered and extracted with DCM (150 mL×2) and concentrated under reduced pressure to give a residue. Then H₂O (100 mL) was added and the pH was adjusted to 4˜5 with 2 M HCl, and extracted with DCM (250 mL×3). The combined organic layers were concentrated under reduced pressure to give a residue. The crude product was purified by reversed-phase HPLC (ACN/0.1% NH₃·H₂O=23%) to afford the title compound (4.56 g, 46% yield) as a yellow solid. ¹H NMR (400 MHZ, DMSO-d₆) δ=7.63 (d, J=9.2 Hz, 1H), 7.52 (dd, J=1.6, 7.0 Hz, 1H), 7.10-7.03 (m, 2H), 6.81 (d, J=9.2 Hz, 1H), 4.94 (s, 2H), 3.76 (t, J=6.0 Hz, 2H), 2.86 (br t, J=6.0 Hz, 2H), 1.54 (s, 9H).

Step 2—Tert-butyl 3-bromo-6-(8-(thiazolo[5,4-b]pyridin-2-ylcarbamoyl)-3,4-dihydroisoquinolin-2 (1H)-yl)picolinate. To a solution of 2-(5-bromo-6-(tert-butoxycarbonyl) pyridin-2-yl)-1,2,3,4-tetrahydroisoquinoline-8-carboxylic acid (4.00 g, 9.23 mmol) and thiazolo[5,4-b]pyridin-2-amine (1.40 g, 9.23 mmol, CAS #31784-70-0) in DMF (40 mL) was added HATU (4.21 g, 11.1 mmol) and DIEA (5.97 g, 46.2 mmol, 8.04 mL). The mixture was then stirred at 25° C., for 12 hr. Next, HATU (8.42 g, 11.1 mmol) and DIEA (11.9 g, 46.2 mmol, 16.1 mL) was added and the mixture was stirred 50° C., for 14 h. On completion, the reaction mixture was quenched with H₂O (50 mL), and then extracted with DCM (150 mL×3). The combined organic layers were washed with brine (100 mL×3), dried over Na₂SO₄, filtered and concentrated under reduced pressure to give a residue. The crude product was triturated with Petroleum ether/Ethyl acetate=10/1 at 25° C., for 2 h to afford the title compound (2.2 g, 41% yield) as a brown solid. ¹H NMR (400 MHz, DMSO-d₆) δ=13.12-12.87 (m, 1H), 8.51 (dd, J=1.2, 4.8 Hz, 1H), 8.15 (dd, J=1.2, 8.1 Hz, 1H), 7.77 (d, J=9.2 Hz, 1H), 7.59 (d, J=7.2 Hz, 1H), 7.52 (dd, J=4.4, 8.1 Hz, 1H), 7.46-7.42 (m, 1H), 7.39-7.34 (m, 1H), 6.88 (d, J=9.2 Hz, 1H), 4.93 (s, 2H), 3.77 (t, J=6.0 Hz, 2H), 3.01 (br t, J=6.0 Hz, 2H), 1.34 (s, 9H).

### 6-((6-(Benzo[d]thiazol-2-ylamino)-5-methylpyridazin-3-yl)(methyl)amino)-3-(1-(cyclohexylmethyl)-5-methyl-1H-pyrazol-4-yl)-N-((4-(((S)-1-((2S,4R)-2-((4-ethynylbenzyl)carbamoyl)-4-hydroxypyrrolidin-1-yl)-3,3-dimethyl-1-oxobutan-2-yl)amino)-4-oxobutyl)sulfonyl)picolinamide (Intermediate IC)

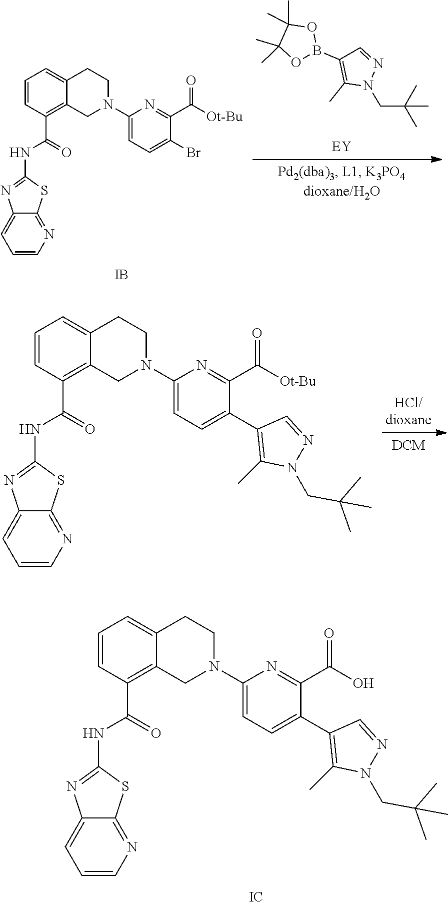

Step 1—Tert-butyl 3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinate. To a solution of tert-butyl 3-bromo-6-(8-(thiazolo[5,4-b]pyridin-2-ylcarbamoyl)-3,4-dihydroisoquinolin-2 (1H)-yl)picolinate (200 mg, 353 umol, Intermediate IB) and 5-methyl-1-neopentyl-4-(4,4,5,5-tetramethyl-1,3,2-dioxaborolan-2-yl)-1H-pyrazole (108 mg, 388 umol, Intermediate EY) in dioxane (16 mL) and H₂O (8 mL) was added Pd₂(dba); (16.2 mg, 17.7 umol), (1S,3R,5R,7S)-1,3,5,7-tetramethyl-8-phenyl-2,4,6-trioxa-8-phosphatricyclo[3.3.1.1^{3,7}]decane (20.6 mg, 70.6 umol, Ll) and K₃PO₄ (150 mg, 706 umol) under N₂. The mixture was stirred at 100° C., for 4 hr. On completion, the reaction mixture was quenched with H₂O (25 mL), and then extracted with DCM (60 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3/1) to afford the title compound (106 mg, 39% yield) as a white solid. ¹H NMR (400 MHZ, DMSO-d₆) δ=13.00 (s, 1H), 8.51 (dd, J=1.2, 4.6 Hz, 1H), 8.15 (d, J=8.0 Hz, 1H), 7.59 (br d, J=7.6 Hz, 1H), 7.54-7.50 (m, 1H), 7.47-7.43 (m, 2H), 7.39-7.35 (m, 1H), 7.22 (s, 1H), 6.94 (d, J=8.8 Hz, 1H), 4.97 (s, 2H), 3.84-3.81 (m, 4H), 3.57-3.56 (m, 1H), 3.03 (br t, J=5.6 Hz, 2H), 2.09 (s, 3H), 1.15 (s, 9H), 0.92 (s, 9H).

Step 2—6-((6-(Benzo[d]thiazol-2-ylamino)-5-methylpyridazin-3-yl)(methyl)amino)-3-(1-(cyclohexylmethyl)-5-methyl-1H-pyrazol-4-yl)-N-((4-(((S)-1-((2S,4R)-2-((4-ethynylbenzyl)carbamoyl)-4-hydroxypyrrolidin-1-yl)-3,3-dimethyl-1-oxobutan-2-yl)amino)-4-oxobutyl)sulfonyl)picolinamide. To a solution of tert-butyl 3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinate (100 mg, 94.1 umol) in DCM (0.4 mL) was added HCl/dioxane (4 M, 390 uL). The mixture was stirred at 25° C., for 12 h. On completion, the reaction mixture was concentrated under reduced pressure to give a residue. The crude product was purified by reversed-phase HPLC (ACN/0.1% NH₃·H₂O=17%) to afford the title compound (27.5 mg, 48% yield) as a yellow solid. LC-MS (ESI⁺) m/z 581 (M+H)⁺; ¹H NMR (400 MHZ, DMSO-d₆) δ=8.50 (br d, J=5.2 Hz, 1H), 8.13 (br d, J=8.0 Hz, 1H), 7.64 (br d, J=7.6 Hz, 1H), 7.53-7.47 (m, 2H), 7.44 (br d, J=8.0 Hz, 1H), 7.39-7.34 (m, 1H), 7.29 (s, 1H), 6.95 (br d, J=9.2 Hz, 1H), 4.96 (s, 2H), 3.89 (br t, J=5.6 Hz, 2H), 3.82 (s, 2H), 3.01 (br t, J=5.2 Hz, 2H), 2.11 (s, 3H), 0.92 (s, 9H).

### 6-(N-(3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid (Intermediate ID)

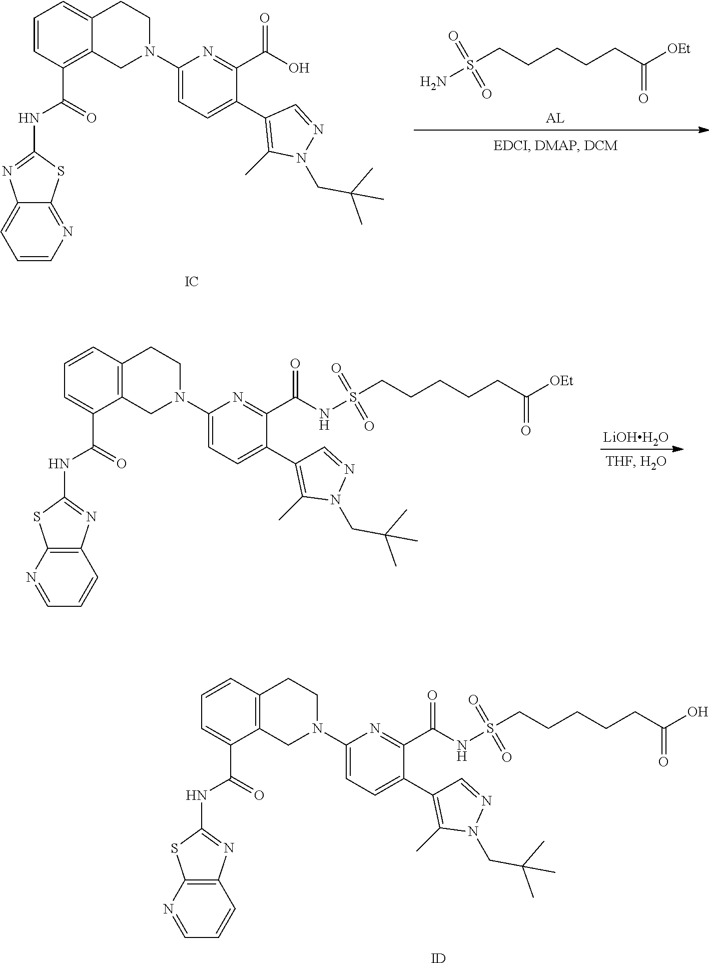

Step 1—Ethyl 6-(N-(3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoate. To a solution of 3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinic acid (1.05 g, 1.70 mmol, HCl, Intermediate IC) and ethyl 6-sulfamoylhexanoate (1.52 g, 6.79 mmol, Intermediate AL) in DCM (10 mL) was added EDCI (651 mg, 3.40 mmol) and DMAP (830 mg, 6.79 mmol). The mixture was stirred at 25° C., for 12 h. On completion, the reaction mixture was quenched with water (10 mL) at 20° C., extracted with DCM (10 mL×2). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=I/O to 0/1) then repurified by reversed-phase HPLC (0.1% FA condition) to give the title compound (480 mg, 36% yield) as a yellow solid. ¹H NMR (400 MHZ, DMSO-d₆) δ=13.05-12.95 (m, 1H), 11.79-11.71 (m, 1H), 8.51 (d, J=4.8 Hz, 1H), 8.17-8.12 (m, 1H), 7.67-7.62 (m, 1H), 7.57-7.50 (m, 2H), 7.47-7.42 (m, 1H), 7.41-7.34 (m, 1H), 7.28 (s, 1H), 7.03 (d, J=8.8 Hz, 1H), 4.98 (s, 2H), 3.96-3.90 (m, 2H), 3.82 (s, 2H), 3.44-3.38 (m, 1H), 3.31-3.23 (m, 3H), 3.03 (br t, J=5.6 Hz, 2H), 2.64-2.55 (m, 1H), 2.27-2.19 (m, 2H), 2.11 (s, 3H), 1.66-1.56 (m, 2H), 1.51-1.43 (m, 2H), 1.36-1.28 (m, 2H), 1.23 (s, 1H), 0.95-0.89 (m, 10H).

Step 2—6-(N-(3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid. To a solution of ethyl 6-(N-(3-(5-methyl-1-neopentyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoate (480 mg, 610 umol) in THF (3.6 mL) and H₂O (1.2 mL) was added LiOH·H₂O (128 mg, 3.05 mmol). The mixture was stirred at 25° C., for 2 h. On completion, the reaction mixture was quenched with water (5 mL) at 20° C., and extracted with DCM (5 mL×2). Then the aqueous layer was adjust to pH=3˜ 4 and extracted with DCM (5 mL×4). The organic layers were washed with brine (15 mL×2), dried over Na₂SO₄, filtered and concentrated under reduced pressure to give a residue. The residue was purified by by prep-TLC (SiO₂, DCM:MeOH=10:1) to give the title compound (430 mg, 93% yield) as a yellow solid. LC-MS (ESI⁺) m/z 759.5 (M+H)⁺.

### 6-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)hexanoic acid (Intermediate IE)

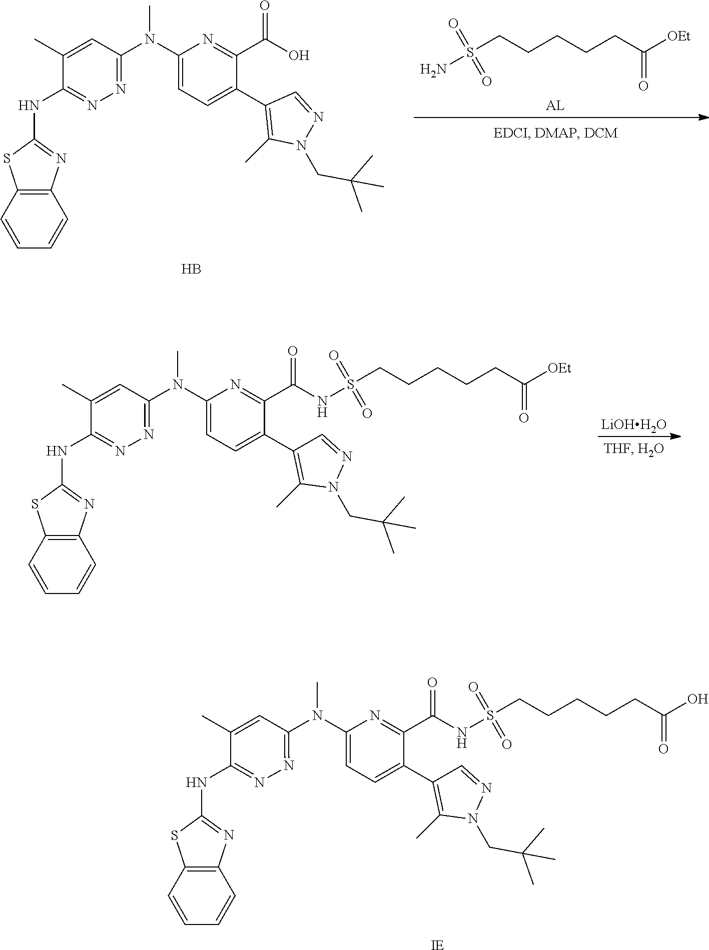

Step 1—Ethyl 6-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)hexanoate. To a solution of 6-((6-(benzo[d]thiazol-2-ylamino)-5-methylpyridazin-3-yl)(methyl)amino)-3-(5-methyl-1-neopentyl-1H-pyrazol-4-yl)picolinic acid (1.1 g, 2.03 mmol, Intermediate HB) and ethyl 6-sulfamoylhexanoate (1.81 g, 8.11 mmol, Intermediate AL) in DCM (20 mL) was added EDCI (777 mg, 4.05 mmol) and DMAP (990 mg, 8.11 mmol) at 20° C., under nitrogen flow. Then the reaction was stirred at 20° C., for 10 h under nitrogen atmosphere. On completion, the reaction was poured into water (40 mL) and extracted with DCM (30 mL×2). The combined organic phase is washed with brine (30 mL×2), dried over Na₂SO₄, then filtered to get the filtrate and concentrated to give a residue. The residue was purified by column chromatography on silica gel (eluted with petroleum ether:ethyl acetate=100:1 to 0:1) to give the title compound (300 mg, 18% yield) as a yellow solid. LC-MS (ESI⁺) m/z 748.5 (M+H)⁺.

Step 2—6-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)hexanoic acid. To a solution of ethyl 6-(N-(6-((6-(benzo[d]thiazol-2-ylamino)-5-methylpyridazin-3-yl)(methyl)amino)-3-(5-methyl-1-neopentyl-1H-pyrazol-4-yl)picolinoyl)sulfamoyl)hexanoate (300 mg, 401 umol) in THF (4.5 mL) and H₂O (1.5 mL) was added LiOH·H₂O (84.1 mg, 2.01 mmol) at 0° C., under nitrogen flow. Then the reaction was stirred at 20° C. for 10 h under nitrogen atmosphere. On completion, the reaction was poured into water (7 mL) and acidified by 2 N hydrochloride acid to pH=4, then extracted with ethyl acetate (10 mL×2). The combined organic phase is washed with brine (5 mL×2), dried over sodium sulfate, filtered to get the filtrate and concentrated to give the title compound (180 mg) as a yellow solid. LC-MS (ESI⁺) m/z 720.5 (M+H)⁺.

### (2S,4R)-1-[(2S)-2-amino-3,3-dimethyl-butanoyl]-N-[(1R)-2-[tert-butyl(dimethyl)silyl]oxy-1-(4-ethynylphenyl)ethyl]-4-hydroxy-pyrrolidine-2-carboxamide (Intermediate IF)

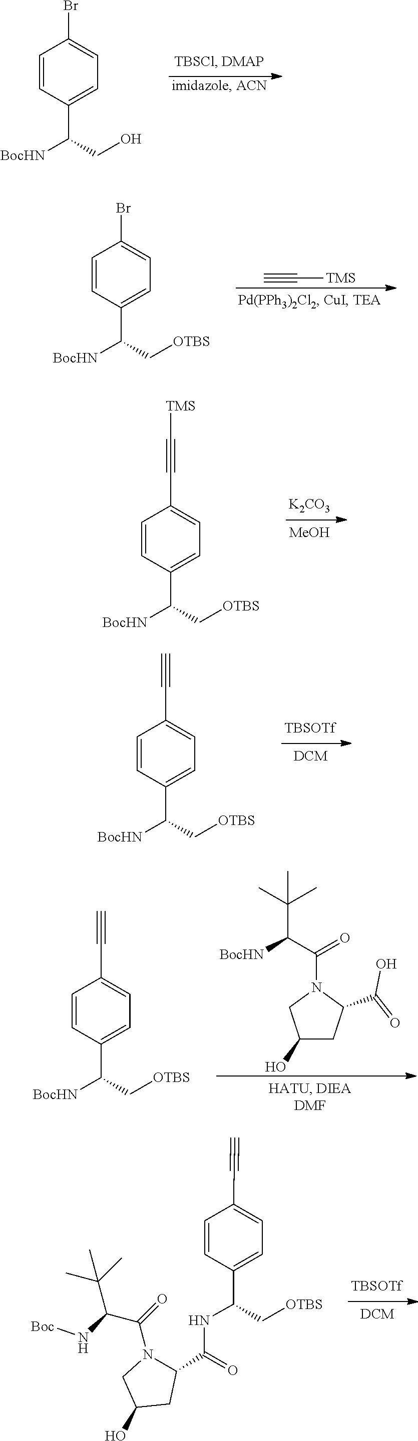

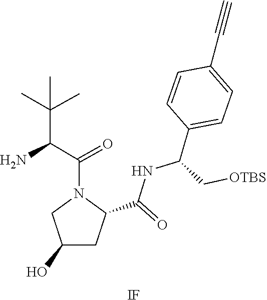 [SMILES: C#Cc1ccc([C@H](CO[Si](C)(C)C(C)(C)C)NC(=O)[C@@H]2C[C@@H](O)CN2C(=O)[C@@H](N)C(C)(C)C)cc1 ; "IF"]

Step 1—(R)-tert-butyl (1-(4-bromophenyl)-2-((tert-butyldimethylsilyl)oxy) ethyl)carbamate. To a solution of (R)-tert-butyl (1-(4-bromophenyl)-2-hydroxyethyl)carbamate (85.0 g, 268 mmol, CAS #849178-85-4) in ACN (850 mL) was added TBSCl (48.6 g, 322 mmol, 39.5 mL), DMAP (39.4 g, 322 mmol) and imidazole (27.4 g, 403 mmol). The mixture was stirred at 25° C., for 1.5 hrs. On completion, the mixture was diluted with EtOAc (500 mL) and the organic layer was washed with saturated NaHCO₃ solution (300 mL×2), brine (200 mL), dried over Na₂SO₄, filtered and concentrated under vacuum to give the title compound (125.5 g) as colorless oil. ¹H NMR (CDCl₃, 400 MHz) & 7.44 (d, J=8.0 Hz, 2H), 7.17 (d, J=8.4 Hz, 2H), 5.28 (s, 2H), 4.64 (s, 1H), 3.84-3.88 (m, 1H), 3.68 (d, J=5.6 Hz, 1H), 1.42 (s, 9H), 0.84 (s, 9H), −0.04 (s, 3H), −0.07 (s, 3H).

Step 2 (R)-tert-butyl (2-((tert-butyldimethylsilyl)oxy)-1-(4-((trimethylsilyl)ethynyl)phenyl)ethyl)carbamate. To a solution of (R)-tert-butyl (1-(4-bromophenyl)-2-((tert-butyldimethylsilyl)oxy) ethyl)carbamate (125 g, 291 mmol) and ethynyl (trimethyl)silane (85.9 g, 874 mmol. 121 mL) in TEA (908 g, 8.98 mol. 1.25 L) was added CuI (2.78 g, 14.5 mmol) and Pd(PPh₃)₂Cl₂ (6.14 g, 8.75 mmol). Then the mixture was stirred at 80° C., for 12 hrs. On completion, the mixture was diluted with EtOAc (500 mL) and water (300 mL), filtered, and separated. The organic layer was washed with brine (200 mL), dried over Na₂SO₄, filtered and concentrated under vacuum. The crude was purified by column chromatography (Petroleum ether/Ethyl acetate=0:1-10:1. Rf=0.5) to give the title compound (110 g, 84% yield) as a light yellow solid. ¹H NMR (CDCl₃, 400 MHz) ò 7.42 (d, J=6.8 Hz, 2H), 7.22 (d, J=8.4 Hz, 2H), 5.28 (s, 2H), 4.69 (s, 1H), 3.85-3.88 (m, 1H), 3.70 (s, 1H), 1.42 (s, 9H), 0.83 (s, 9H), 0.25 (s. 9H), −0.06 (s, 3H), −0.09 (s, 3H).

Step 3—(R)-tert-butyl (2-((tert-butyldimethylsilyl)oxy)-1-(4-ethynylphenyl)ethyl)carbamate. To a solution of (R)-tert-butyl (2-((tert-butyldimethylsilyl)oxy)-1-(4-((trimethylsilyl)ethynyl)phenyl)ethyl)carbamate (105 g, 234 mmol) in MeOH (700 mL) was added K₂CO; (64.8 g, 469 mmol) and the mixture was stirred at 25° C., for 2 hrs. On completion, the mixture was diluted with water (1000 mL), and extracted with EtOAc (300 mL×2). The combined organic layer was washed with brine (200 mL), dried over Na₂SO₄, filtered and concentrated under vacuum to give the title compound (85.5 g, 97% yield) as yellow oil. ¹H NMR (CDCl₃, 400 MHz) $7.45 (d, J=8.4 Hz, 2H), 7.25 (d, J=8.0 Hz, 2H), 5.30 (s, 1H), 4.69 (s, 1H), 3.85-3.89 (m, 1H), 3.71 (s, 1H), 3.05 (s, 1H), 1.43 (s, 9H), 0.83 (s, 9H). −0.06 (s, 3H), −0.09 (s, 3H).

Step 4—(R)-2-((tert-butyldimethylsilyl)oxy)-1-(4-ethynylphenyl)ethanamine. To a solution of (R)-tert-butyl (2-((tert-butyldimethylsilyl)oxy)-1-(4-ethynylphenyl)ethyl)carbamate (70.0 g, 186 mmol) in DCM (700 mL) was added TBSOTf (59.1 g, 223 mmol. 51.4 mL), and the mixture was stirred at 25° C., for 30 min. On completion, the mixture was diluted with water (100 mL), adjusted to pH=7 with saturated NaHCO₃, and separated. The organic layer was washed with brine (100 mL), dried over Na₂SO₄, filtered and concentrated under vacuum. The crude was purified by column chromatography (DCM/MeOH=0:1-50:1. Rf=0.4) to give the title compound (51.0 g, 99% yield) as yellow oil. ¹H NMR (CDCl₃, 400 MHZ) δ 7.45 (d, J=8.4 Hz, 2H), 7.33 (d, J=8.4 Hz, 2H), 4.07 (dd, J=4.0 Hz, 8.0 Hz, 1H), 3.70 (dd, J=4.0 Hz. 9.6 Hz, 1H), 3.50 (dd, J=8.4 Hz, 10.0 Hz, 1H), 3.05 (s, 1H), 0.89 (s, 9H), 0.02 (s. 6H).

Step 5—tert-butyl ((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl) carbamate. To a solution of (2S,4R)-1-((S)-2-((tert-butoxycarbonyl)amino)-3,3-dimethylbutanoyl)-4-hydroxypyrrolidine-2-carboxylic acid (57.5 g, 166 mmol. CAS #630421-46-4) in DMF (450 mL) was added HATU (76.1 g. 200 mmol) and DIEA (64.7 g, 500 mmol. 87.2 mL) at 0-5° C. The mixture was stirred at 0-5° C., for 30 min, and then (R)-2-((tert-butyldimethylsilyl)oxy)-1-(4-ethynylphenyl)ethanamine (46.0 g, 166 mmol) was added at 0-5° C. The mixture was stirred at 25° C., for 1 hr. On completion, the mixture was diluted with water (1500 mL), and extracted with DCM (500 mL×2). The combined organic layer was washed with brine (500 mL), dried over Na₂SO₄, filtered and concentrated under vacuum. The crude product was purified by reverse phase HPLC (0.1% NH₃·H₂O) to give the title compound (66.0 g, 66% yield) as a white solid. LC-MS (ESI⁺) m/z 602.5 (M+H)⁺.

Step 6-(2S,4R)-1-((S)-2-amino-3,3-dimethylbutanoyl)-N-((R)-2-((tert-butyldimethylsilyl)oxy)-1-(4-ethynylphenyl)ethyl)-4-hydroxypyrrolidine-2-carboxamide. To a solution of tert-butyl ((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl) carbamate (70.0 g, 116 mmol) in DCM (600 mL) was added HCl/dioxane (4M, 100 mL) and the mixture was stirred at 25° C., for 0.5 hr. On completion, the mixture was diluted with water (100 mL), adjusted to pH=7 with saturated NaHCO₃, and separated. The organic layer was washed with brine (300 mL), dried over Na₂SO₄, filtered and concentrated under vacuum. The crude was purified by pre-HPLC (column: Kromasil Eternity XT 250*80 mm*10 um; mobile phase: [water (ammonia hydroxide v/v)-ACN]: B %: 45%-75%, 20 min) to give the title compound (20.5 g, 35% yield) as an off-white solid. LC-MS (ESI⁺) m/z 502.5 (M+H)⁺. ¹H NMR (CDCl₃, 400 MHz) ò 7.47 (d, J=8.0 Hz, 2H), 7.31 (d, J=8.0 Hz, 2H), 7.17 (d, J=8.0 Hz, 1H), 5.06-5.11 (m, 1H), 4.56 (t, J=7.2 Hz, 2H), 3.88-3.89 (m, 1H), 3.70-3.78 (m, 2H), 3.54 (d, J=2.0 Hz, 1H), 3.29 (s, 1H), 3.07 (s, 1H), 2.34-2.39 (m, 1H), 1.96-2.01 (m, 1H), 1.00 (s, 9H), 0.87 (s, 9H), 0.08 (s, 6H).

### (S)-2-(1-aminoethyl)-5-ethynylphenol (Intermediate IG)

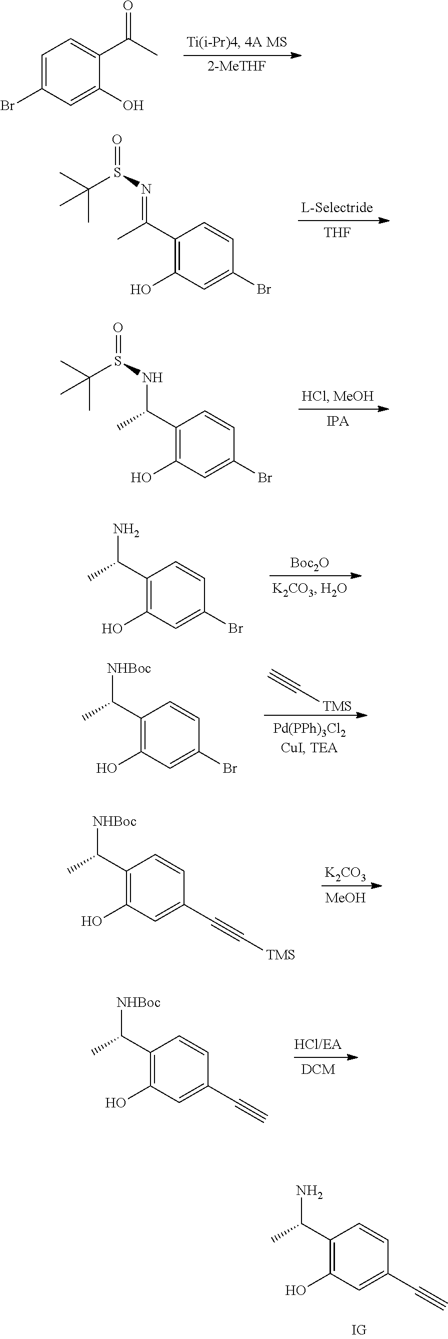

Step 1—(R,E)-N-(1-(4-bromo-2-hydroxyphenyl)ethylidene)-2-methylpropane-2-sulfinamide. To a solution of 1-(4-bromo-2-hydroxy-phenyl)ethanone (10 g, 46.5 mmol) and (R)-2-methylpropane-2-sulfinamide (16.9 g, 139 mmol) in 2,5-dimethylfuran (10 mL) was added Ti (i-PrO)4 (39.6 g, 139 mmol, 41.2 mL) and 4A molecular sieves (500 mg) at 0° C. The mixture was then stirred at 80° C., for 2 hr. On completion, the reaction mixture was filtered to give the solution and then diluted with H₂O (10 mL) and extracted with EA (10 mL×3). The combined organic layers were washed with NaCl (10 mL×1), dried over Na₂SO₄, filtered and concentrated under reduced pressure to give a residue. The mixture was purified by MPLC (SiO₂, PE:EA=10:1 to 5:1) to give the title compound (7 g, 40% yield) as a white solid. LC-MS (ESI) m/z 319.9 (M+H).

Step 2—(R)-N-((S)-1-(4-bromo-2-hydroxyphenyl)ethyl)-2-methylpropane-2-sulfinamide. A mixture of (NE,R)—N-[1-(4-bromo-2-hydroxy-phenyl)ethylidene]-2-methyl-propane-2-sulfinamide (5.7 g. 17.9 mmol) in THF (50 mL) was degassed and purged with N₂ 3 times. Then L-selectride (1 M, 53.7 mL) was added at 0° C., and then the mixture was stirred at 0-25° C., for 3 hr under N₂ atmosphere. On completion, the mixture was quenched with H₂O (50 mL at 0° C., and then extracted by EA (100 mL×3). The combined organic layer was washed with NaCl (100mLx 3) and dried over Na₂SO₄, then the mixture was concentrated under reduce pressure to give a residue. The residue was purified by column chromatography (SiO₂. Petroleum ether/Ethyl acetate=20/1 to 3/1) to give the title compound (1 g, 17% yield) as a yellow solid. LC-MS (ESI) m/z 322.0 (M+H+2).

Step 3—(S)-2-(1-aminoethyl)-5-bromophenol. To a solution of (R)-N-[(1S)-1-(4-bromo-2-hydroxy-phenyl)ethyl]-2-methyl-propane-2-sulfinamide (0.5 g, 1.56 mmol) in MeOH (4 mL) was added HCl (12 M, 1.03 mL) in IPA (1 mL). The mixture was then stirred at 25° C., for 1 hr. On completion, the mixture was concentrated under reduce pressure to give the title compound (0.35 g). LC-MS (ESI) m/z 200.8 (M-NH₂)

Step 4—(S)-tert-butyl (1-(4-bromo-2-hydroxyphenyl)ethyl)carbamate. To a solution of 2-[(1S)-1-aminocthyl]-5-bromo-phenol (0.35 g, 1.62 mmol) in H₂O (3 mL) was added K₂CO₃ (1.34 g, 9.72 mmol) and Boc₂O (393 mg, 1.80 mmol, 414 uL). The mixture was stirred at 25° C., for 12 hr. On completion, the mixture was quenched with H₂O (50 mL) at 0° C., and then extracted with EA (100 mL×3). The combined organic layer was washed with NaCl (100 mL×3), dried over Na₂SO₄, then the mixture was concentrated under reduce pressure to give the title compound (330 mg) as a white solid. LC-MS (ESI) m/z 199.0 (M-Boc-NH₂).

Step 5—(S)-tert-butyl (1-(2-hydroxy-4-((trimethylsilyl)ethynyl)phenyl)ethyl)carbamate. A mixture of tert-butyl N-[(1S)-1-(4-bromo-2-hydroxy-phenyl)ethyl]carbamate (330 mg, 1.04 mmol), ethynyl (trimethyl)silane (615 mg. 6.26 mmol, 868 uL), CuI (19.9 mg, 104 umol), and Pd(PPh₂)₂Cl₂ (36.6 mg. 52.2 umol) in TEA (8 mL) was degassed and purged with N₂ three times. Then the mixture was stirred at 70° C., for 12 hr under N₂ atmosphere. On completion, the reaction mixture was diluted with H₂O (20 mL) and extracted with EA (20 mL×3). The combined organic layers were washed with NaCl (10 mL×3), dried over Na₂SO₄, filtered and concentrated under reduced pressure to give a residue. The residue was purified by column chromatography (SiO₂, Petroleum ether/Ethyl acetate=40/1 to 5/1) to give the title compound (150 mg, 43% yield) as a white solid. LC-MS (ESI⁺) m/z 356.1 (M+23)⁺.

Step 6-(S)-tert-butyl (1-(2-hydroxy-4-((trimethylsilyl)ethynyl)phenyl)ethyl)carbamate. To a solution of tert-butyl N-[(1S)-1-[2-hydroxy-4-(2-trimethylsilylethynyl)phenyl]ethyl]carbamate (150 mg, 450 umol) in MeOH (2 mL) was added K₂CO; (124 mg, 899 umol). The mixture was then stirred at 25° C. for 1 hr. On completion, the reaction mixture was diluted with EA (150 mL) and extracted with H₂O (50 mL×3). The combined organic layer was washed with NaCl (40 mL×30), dried over Na₂SO₄, filtered and concentrated under reduced pressure to give a residue. The residue was purified by column chromatography (SiO₂, Petroleum ether/Ethyl acetate=100/1 to 10/1) to give the title compound (100 mg, 83% yield) as a white solid. LC-MS (ESI⁺) m/z 284.2 (M+23)⁺.

Step 7-(S)-2-(1-aminoethyl)-5-ethynylphenol. To a solution of tert-butyl N-[(1S)-1-(4-ethynyl-2-hydroxy-phenyl)ethyl]carbamate (30 mg, 100 umol) in DCM (1 mL) was added HCl/dioxane (4 M, 0.2 mL). The mixture was then stirred at 25° C., for 1 hr. On completion, the reaction mixture was concentrated under reduced pressure to give the title compound (30 mg) as a white solid. LC-MS (ESI⁺) m/z 145.3 (M-NH₂)⁺.

### (2S,4R)-1-((2S)-2-(6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanamido)-3,3-dimethylbutanoyl)-4-hydroxypyrrolidine-2-carboxylic acid (Intermediate IH)

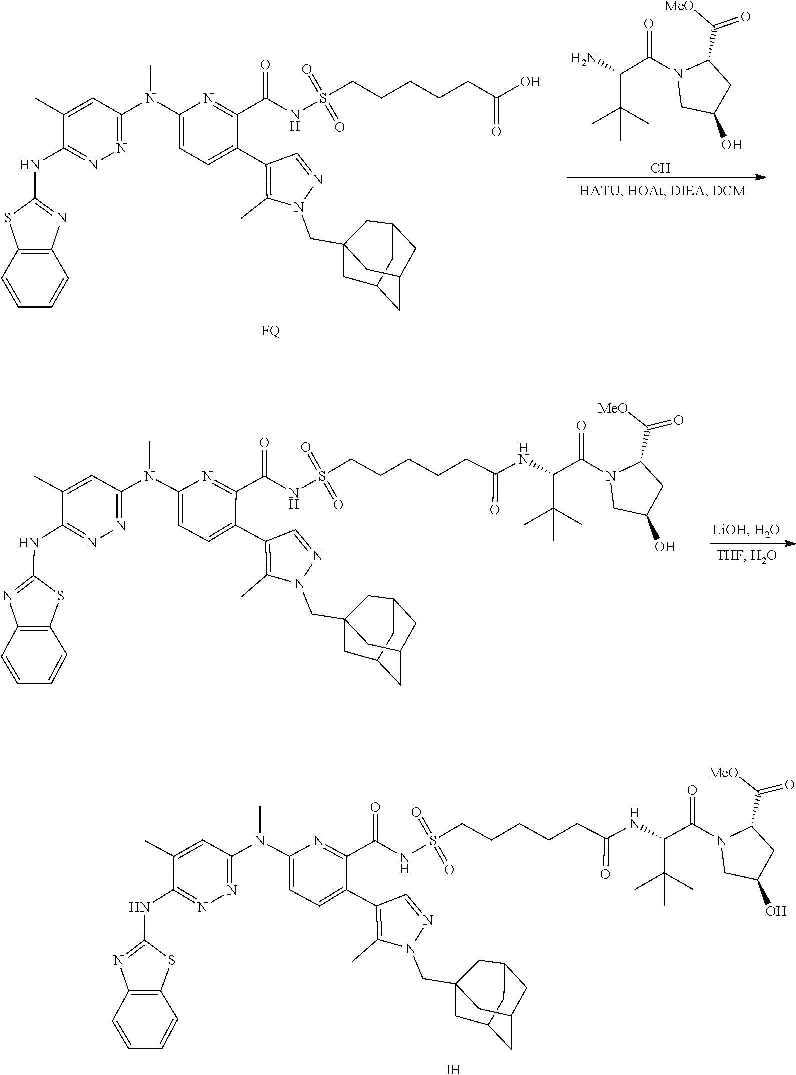

Step 1—(2S,4R)-methyl 1-((2S)-2-(6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanamido)-3,3-dimethylbutanoyl)-4-hydroxypyrrolidine-2-carboxylate. To a solution of 6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanoic acid (500 mg, 627 umol, Intermediate FQ), (2S,4R)-methyl 1-((S)-2-amino-3,3-dimethylbutanoyl)-4-hydroxypyrrolidine-2-carboxylate (443 mg, 1.50 mmol, HCl, Intermediate CH), HATU (262 mg, 689 umol), DIEA (405 mg, 3.13 mmol) and HOAt (111 mg, 815 umol) in DCM (5 mL) was degassed and purged with N₂ three times. Then the mixture was stirred at −10° C., for 1 h under N₂ atmosphere. On completion, the reaction mixture was quenched with H₂O (10 mL), and then extracted with DCM (10 mL×3). The combined organic layers were washed with brine (10 mL×3), dried over Na₂SO₄, filtered and concentrated under reduced pressure to give a residue. The residue was purified by reversed-phase HPLC (0.1% FA condition, 60%-80%) to give the title compound (580 mg, 89% yield) as a yellow solid. LC-MS (ESI⁺) m/z 519.9 (M/2+H)⁺.

Step 2—(2S,4R)-1-((2S)-2-(6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanamido)-3,3-dimethylbutanoyl)-4-hydroxypyrrolidine-2-carboxylic acid. A solution of (2S,4R)-methyl 1-((2S)-2-(6-(N-(3-(1-((1s,3s)-adamantan-1-ylmethyl)-5-methyl-1H-pyrazol-4-yl)-6-((6-(benzo[d]thiazol-2-ylamino)-5-methylpyridazin-3-yl)(methyl)amino)picolinoyl)sulfamoyl)hexanamido)-3,3-dimethylbutanoyl)-4-hydroxypyrrolidine-2-carboxylate (580 mg, 559 umol) and LiOH·H₂O (117 mg, 2.79 mmol) in THF (4 mL) and H₂O (2 mL) was degassed and purged with N₂ three times, and then the mixture was stirred at 25° C., for 1 h under N₂ atmosphere. On completion, the reaction mixture was quenched with H₂O (10 mL), and then extracted with DCM (10 mL×3). The combined aqueous phase was treated with HCl (2M) to adjusted pH=3˜4, then the mixture was extracted by DCM (10 mL×3). The combined organic phase was concentrated under reduced pressure to give the title compound (500 mg) as a yellow solid. LC-MS (ESI⁺) m/z 513.1 (M/2+H)⁺.

### (R)-1-(4-ethynylphenyl)-2-methoxyethanamine (Intermediate II)

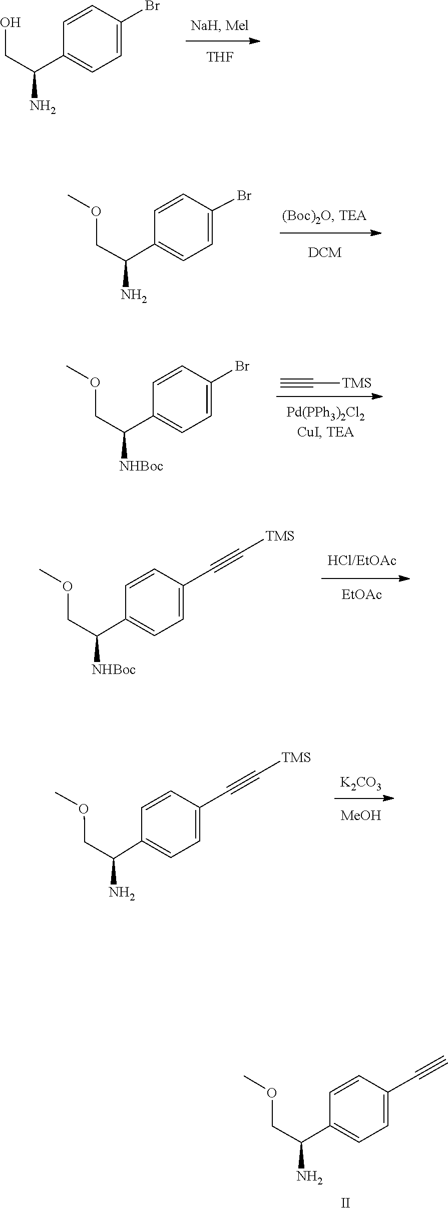

Step 1—(R)-1-(4-bromophenyl)-2-methoxyethanamine. To a solution of (R)-2-amino-2-(4-bromophenyl)ethanol (1 g, 4.63 mmol, CAS #354153-64-3) in THF (10 mL) was added NaH (370 mg, 9.26 mmol, 60% dispersion in mineral oil) and Mel (689 mg, 4.86 mmol) at 0° C., under nitrogen flow. Then the reaction was stirred at 20° C., for 10 h under nitrogen atmosphere. On completion, the reaction was poured into NH₄Cl (sat.aq, 30 mL) and extracted with EtOAc (20 mL×2). The combined organic phase was washed with brine (20 mL×2), dried over Na₂SO₄, then filtered to get the filtrate and concentrated to give a residue. The crude product was purified by reversed-phase HPLC (0.1% NH₃·H₂O condition) to give the title compound (600 mg. 56% yield) as a white solid. ¹H NMR (400 MHZ. DMSO-d₆) ô=7.48 (d, J=8.4 Hz. 2H), 7.33 (d, J=8.4 Hz, 2H), 4.00 (dd, J=5.6, 6.8 Hz, 1H), 3.32-3.26 (m, 2H), 3.23 (s, 3H), 2.19-1.82 (m. 2H).

Step 2—(R)-2-amino-2-(4-bromophenyl)ethanol compound with (R)-1-(4-bromophenyl)-2-methoxyethanamine. To a solution of (R)-1-(4-bromophenyl)-2-methoxyethanamine (500 mg. 2.17 mmol) and Boc₂O (711 mg. 3.26 mmol) in DCM (10 mL) was added TEA (440 mg. 4.35 mmol. 605 uL) at 20° C. under nitrogen flow. Then the reaction was stirred at 20° C., for 10 h under nitrogen atmosphere. On completion, the reaction was concentrated to give a residue. The residue was purified by column chromatography on silica gel (eluted with petroleum ether:ethyl acetate=100:1 to 7:3) to give the title compound (680 mg. 95% yield) as a yellow solid. ¹H NMR (400 MHz. CDCl₃) δ=7.38 (d, J=8.4 Hz, 2H). 7.12 (d, J=8.4 Hz, 2H), 5.22 (br s, 1H), 4.68 (br s, 1H), 3.67-3.40 (m, 2H), 3.26 (s, 3H), 1.34 (br s. 8H).

Step 3—(R)-tert-butyl (2-methoxy-1-(4-((trimethylsilyl)ethynyl)phenyl)ethyl)carbamate. To a solution of (R)-tert-butyl (1-(4-bromophenyl)-2-methoxyethyl)carbamate (670 mg. 2.03 mmol) and ethynyltrimethylsilane (1.99 g, 20.2 mmol) in TEA (10 mL) was added Pd(PPh₃)₂Cl₂ (71.2 mg, 101 umol) and CuI (38.6 mg. 202 umol) at 20° C., under nitrogen flow. Then the reaction was stirred at 80° C., for 10 h under nitrogen atmosphere. On completion, the reaction was poured into water (20 mL) and extracted with EtOAc (30 mL×2). The combined organic phase is washed with brine (10 mL×2), dried over Na₂SO₄, then filtered to get the filtrate and concentrated to give a residue. The residue was purified by column chromatography on silica gel (eluted with petroleum ether:ethyl acetate=100:1 to 100:30) to give the title compound (600 mg. 85% yield) as a yellow solid. ¹H NMR (400 MHZ. CDCl₃) ¿=7.43 (d, J=8.4 Hz. 2H), 7.25 (d, J=8.0 Hz, 2H), 3.65-3.45 (m, 2H), 3.33 (s, 3H), 1.42 (br s. 9H), 0.25 (s, 9H).

Step 4—(R)-2-methoxy-1-(4-((trimethylsilyl)ethynyl)phenyl)ethanamine. To a solution of (R)-tert-butyl (2-methoxy-1-(4-((trimethylsilyl)ethynyl)phenyl)ethyl)carbamate (0.30 g, 863 umol) in EtOAc (3 mL) was added HCl/EtOAc (0.80 M. 3 mL) at 20° C., under nitrogen flow. Then the reaction was stirred at 20° C., for 20 h under nitrogen atmosphere. On completion, the reaction was concentrated to give the tittle compound (0.25 g) as a yellow solid. ¹H NMR (400 MHZ. CDCl₃) δ=8.82 (br s, 2H), 7.48 (s, 3H), 4.40 (br s, 1H), 3.82 (br d, J=6.4 Hz, 1H), 3.71 (br d, J=7.6 Hz, 1H), 3.37 (s, 3H), 2.11 (s, 2H), 0.26 (s. 7H).

Step 5—(R)-1-(4-ethynylphenyl)-2-methoxyethanamine. To a solution of (R)-2-methoxy-1-(4-((trimethylsilyl)ethynyl)phenyl)ethanamine (250 mg, 1.01 mmol) in MeOH (6 mL) was added K₂CO₃ (279 mg. 2.02 mmol) at 20° C., under nitrogen flow. Then the reaction was stirred at 20° C., for 10 h under nitrogen atmosphere. On completion, the reaction was filtered to get the filtrate and concentrated to give a residue. The crude product was purified by reversed-phase HPLC (0.1% FA condition) to give the tittle compound (60.0 mg. 30% yield) as a white solid. LC-MS (ESI) m/z 159.4 (M-16).

### (S)-2-(2-(1-aminoethyl)-5-ethynylphenoxy) ethanol (Intermediate IJ)

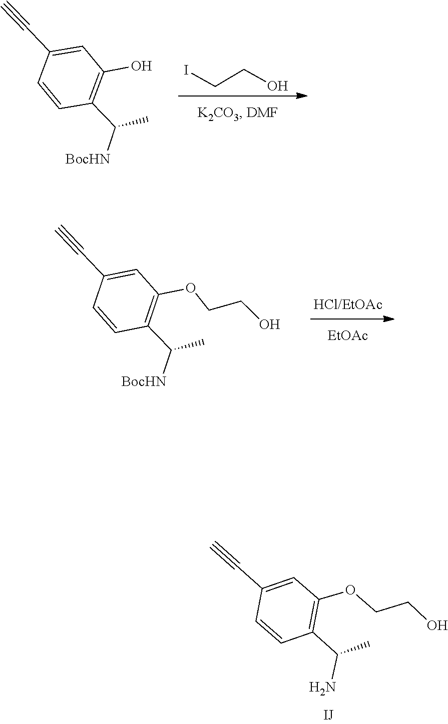

Step 1—(S)-tert-butyl (1-(4-ethynyl-2-(2-hydroxyethoxy)phenyl)ethyl)carbamate. A mixture of(S)-tert-butyl (1-(4-ethynyl-2-hydroxyphenyl)ethyl)carbamate (490 mg, 1.88 mmol, synthesized via Steps 1-6 of Intermediate IG), K₂CO; (777 mg, 5.63 mmol) in DMF (5 mL). The mixture was stirred at 25° C., for 0.5 hr under N₂ atmosphere and then 2-iodoethanol (484 mg, 2.81 mmol, 220 uL, CAS #624-76-0) was added and the mixture was stirred at 80° C., for 12 h. On completion, the reaction mixture was quenched with water (5 mL) at 20° C., and extracted with EtOAc (10 mL×4). The combined organic layers were washed with brine (50 mL×2), dried over Na₂SO₄, filtered and concentrated under reduced pressure to give a residue. The residue was purified by flash silica gel chromatography (ISCOR: 12 g SepaFlash® Silica Flash Column, Eluent of 0˜24% Ethyl acetate/Petroleum ether gradient @ 40 mL/min) to give the title compound (350 mg, 61% yield) as a brown solid. ¹H NMR (400 MHZ, DMSO-d₆) δ=7.33 (br d, J=8.8 Hz, 1H), 7.22 (d, J=8.0 Hz, 1H), 7.06-7.00 (m, 2H), 4.98-4.89 (m, 1H), 4.85 (t, J=5.6 Hz, 1H), 4.48 (s, 1H), 4.11 (s, 1H), 4.03 (br s, 1H), 3.72 (q, J=5.2 Hz, 2H), 1.35 (s, 9H), 1.23 (br d, J=6.8 Hz, 3H).

Step 2—(S)-2-(2-(1-aminoethyl)-5-ethynylphenoxy) ethanol. To a solution of(S)-tert-butyl (1-(4-ethynyl-2-(2-hydroxyethoxy)phenyl)ethyl)carbamate (350 mg, 1.15 mmol) in EtOAC (3.5 mL) was added HCl/EtOAc (0.8 M, 3.5 mL). The mixture was stirred at 25° C., for 12 h. On completion, the reaction mixture was concentrated under reduced pressure to give a residue. The crude product was triturated with EtOAc at 25° C., for 30 min to give the title compound (90.0 mg, 38% yield) as a white solid. LC-MS (ESI⁺) m z 189.0 (M-16)⁺.

### 6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid (Intermediate IK)

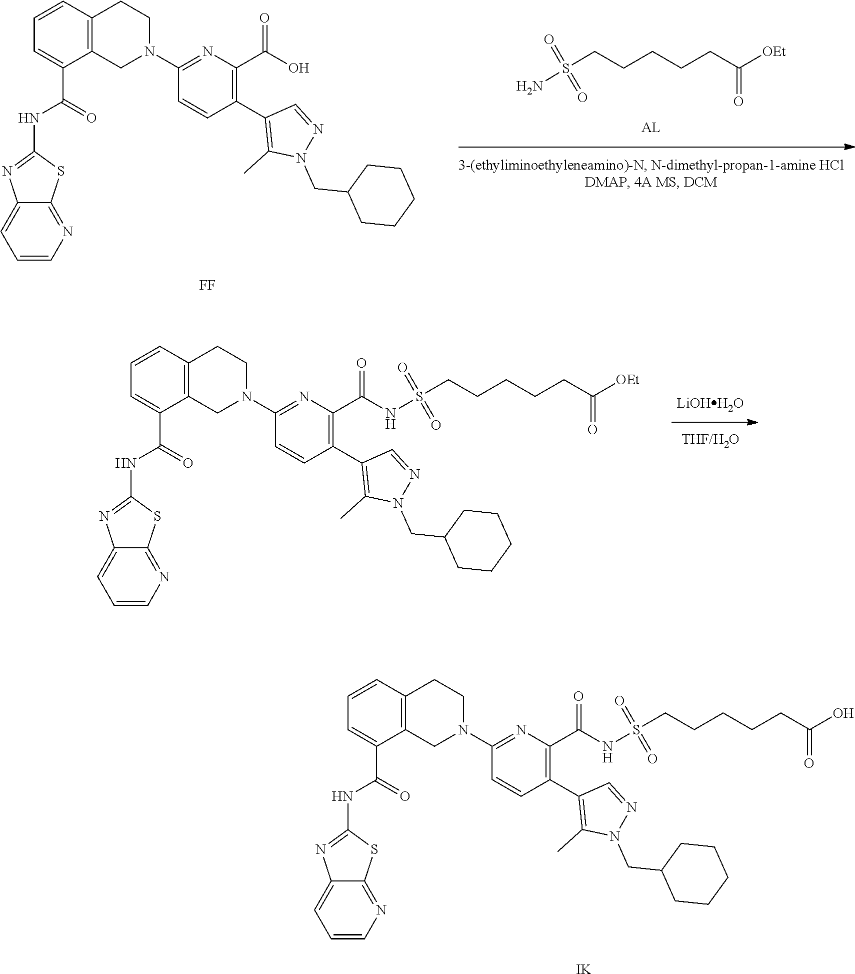

Step 1—Ethyl 6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoate. To a stirred solution of ethyl 6-sulfamoylhexanoate (10.9 g, 48.8 mmol, Intermediate AL), 3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinic acid (9.9 g, 16.2 mmol, Intermediate FF) in DCM (30 mL) at 25° C., was added DMAP (4.98 g, 40.7 mmol), 3-(ethyliminomethyleneamino)-N,N-dimethyl-propan-1-amine hydrochloride (6.25 g, 32.5 mmol), 4 Å molecular sieves (200 mg, 16.2 mmol) and the mixture was stirred at 25° C., for 6 h. On completion, the mixture was filtered and the filter liquor was quenched with H₂O (30 mL) and extracted with DCM (60 mL×3). The combined organic layer was dried over Na₂SO₄ and evaporated. The residue was purified by silica gel column chromatography (PE:EA=1:0˜1:1) to give the title compound (7 g, 53% yield) was orange solid. LC-MS (ESI⁺) m/z 813.3 (M+H)⁺.

Step 2—6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid. A solution of ethyl 6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoate (7 g, 8.61 mmol) in THF (30 mL) and H₂O (30 mL) was added LiOH·H₂O (1.81 g, 43.1 mmol), then the mixture was stirred at 25° C., for 12 h. On completion, the reaction mixture was quenched with H₂O (50 mL) at 20° C., and extracted with DCM (50 mL×3). Then the aqueous phase was adjusted to pH=3˜4, and then extracted with DCM (50 mL×3). The organic layer was washed with brine (50 mL×2), dried over Na₂SO₄, filtered and concentrated under reduced pressure to the title compound (4 g, 59% yield) as yellow solid. LC-MS (ESI⁺) m/z 785.5 (M+H)⁺.

### (2S,4R)-1-((S)-2-(6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanamido)-3,3-dimethylbutanoyl)-4-hydroxypyrrolidine-2-carboxylic (Intermediate IL) acid

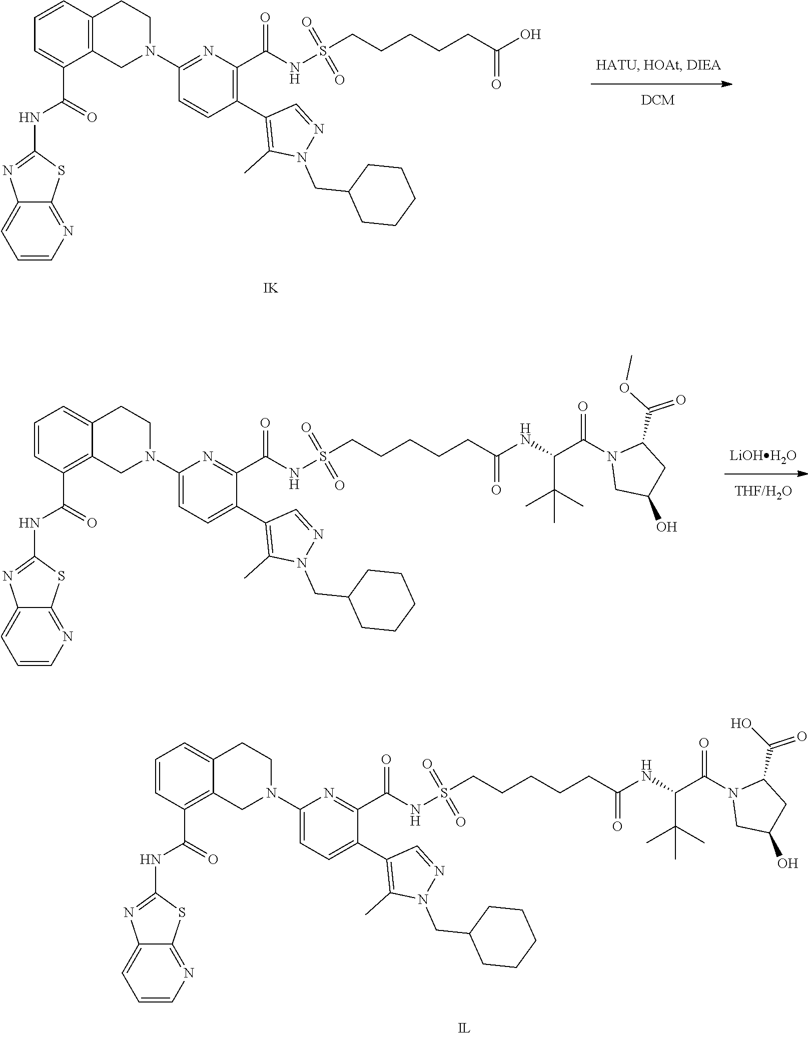 [SMILES: Cc1c(-c2ccc(N3CCc4cccc(C(=O)Nc5nc6cccnc6s5)c4C3)nc2C(=O)NS(=O)(=O)CCCCCC(=O)O)cnn1CC1CCCCC1 ; "IK" ; COC(=O)[C@@H]1C[C@@H](O)CN1C(=O)[C@@H](NC(=O)CCCCCS(=O)(=O)NC(=O)c1nc(N2CCc3cccc(C(=O)Nc4nc5cccnc5s4)c3C2)ccc1-c1cnn(CC2CCCCC2)c1C)C(C)(C)C ; "LiOH·H2O" ; Cc1c(-c2ccc(N3CCc4cccc(C(=O)Nc5nc6cccnc6s5)c4C3)nc2C(=O)NS(=O)(=O)CCCCCC(=O)N[C@H](C(=O)N2C[C@H](O)C[C@H]2C(=O)O)C(C)(C)C)cnn1CC1CCCCC1]

Step 1—(2S,4R)-methyl 1-((S)-2-(6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanamido)-3,3-dimethylbutanoyl)-4-hydroxypyrrolidine-2-carboxylate. To a solution of 6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanoic acid (3.8 g, 4.84 mmol, Intermediate IK), (2S,4R)-methyl 1-((S)-2-amino-3,3-dimethylbutanoyl)-4-hydroxypyrrolidine-2-carboxylate (1.25 g, 4.84 mmol, Intermediate CH), and HOAt (856 mg, 6.29 mmol, 880 uL) in DCM (130 mL) was added DIEA (3.13 g, 24.2 mmol, 4.22 mL) and the mixture was cooled to −10° C. Then HATU (2.02 g, 5.33 mmol) was added and the mixture was stirred at −10° C., for 0.5 h. On completion, the reaction mixture was quenched with H₂O (120 mL) at 20° C., and extracted with DCM (120 mL×3). The combined organic layers were washed with brine (120 mL×2), dried over Na₂SO₄, filtered and concentrated under reduced pressure to give a residue. The residue was purified by column chromatography (SiO₂, Petroleum ether/Ethyl acetate=1/1 to DCM/Ethyl acetate=1/1) to give the title compound (3.1 g, 63% yield) as yellow solid. LC-MS (ESI⁺) m/z 1025.8 (M+H)⁺.

Step 2—(2S,4R)-1-((S)-2-(6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanamido)-3,3-dimethylbutanoyl)-4-hydroxypyrrolidine-2-carboxylic acid. To a solution of (2S,4R)-methyl 1-((S)-2-(6-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)hexanamido)-3,3-dimethylbutanoyl)-4-hydroxypyrrolidine-2-carboxylate (3.1 g, 3.02 mmol in THF (15 mL) and H₂O (15 mL) was added LiOH·H₂O (637 mg, 15.1 mmol), then the mixture was stirred at 25° C., for 12 h. On completion, the reaction mixture was quenched with H₂O (50 mL) at 20° C., and extracted with DCM (50 mL×3). Then the aqueous phase was adjusted to pH=3˜4, and then extracted with DCM (50 mL×3). The organic layers were washed with brine (50 mL×2), dried over Na₂SO₄, filtered and concentrated under reduced pressure to give the title compound (2.84 g, 93% yield) as yellow solid. LC-MS (ESI⁺) m/z 1011, 7 (M+H)⁺.

### 1-(4-Ethynylphenyl)cyclopropanamine (Intermediate IM)

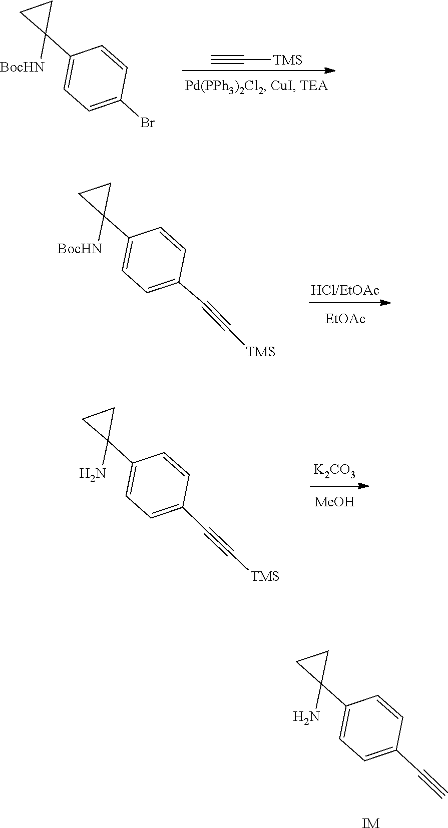

Step 1—Tert-butyl (1-(4-((trimethylsilyl)ethynyl)phenyl)ethyl)carbamate. To a solution of tert-butyl (1-(4-bromophenyl)cyclopropyl) carbamate (1.00 g, 3.20 mmol, CAS #360773-84-8) and ethynyltrimethylsilane (3.15 g, 32.0 mmol, 4.44 mL, CAS #1066-54-2) in TEA (10 mL) was added Pd(PPh₃)₂Cl₂ (225 mg, 320 umol) and CuI (122 mg, 641 umol). The mixture was then stirred at 85° C., for 7 h under N₂ atmosphere. On completion, the reaction mixture was quenched with H₂O (2 mL), and then extracted with EtOAc (2 mL×3). The combined organic layers were washed with brine (2 mL×3), dried over Na₂SO₄, filtered and concentrated under reduced pressure to give a residue. The residue was purified by column chromatography (SiO₂, Petroleum ether/Ethyl acetate=99/1 to 1/9) to give the title compound (1.00 g, 95% yield) as a white solid. ¹H NMR (400 MHZ, DMSO-d₆) δ=7.71 (s, 1H), 7.35 (br d, J=8.0 Hz, 2H), 7.09 (br d, J=8.0 Hz, 2H), 1.37 (s, 9H), 1.14 (br d, J=3.2 Hz, 4H), 0.21 (s, 9H).

Step 2—1-(4-((Trimethylsilyl)ethynyl)phenyl)ethanamine. A mixture of tert-butyl (1-(4-((trimethylsilyl)ethynyl)phenyl)ethyl)carbamate (1.00 g, 3.03 mmol), HCl/EtOAc (4 M, 1 mL) in EtOAc (9 mL) was degassed and purged with N₂ three times, and then the mixture was stirred at 25° C., for 10 h under N₂ atmosphere. On completion, the reaction mixture was quenched with H₂O (9 mL), and then extracted with EtOAc (9 mL×3). The combined organic layers were washed with brine (9 mL×3), dried over Na₂SO₄, filtered and concentrated under reduced pressure to give a residue. The combined organic phase was concentrated under reduced pressure to give the title compound (0.80 g) as a white solid. 1H NMR (400 MHZ, DMSO-d₆) δ=8.90-8.83 (m, 2H), 7.49 (d, J=8.4 Hz, 2H), 7.38 (d, J=8.4 Hz, 2H), 1.41 (br s, 2H), 1.25-1.20 (m, 2H), 0.23 (s, 9H).

Step 3—1-(4-Ethynylphenyl)cyclopropanamine. To a 1-(4-((trimethylsilyl)ethynyl)phenyl)ethanamine (700 mg, 3.05 mmol) in MeOH (7 mL) was added K₂CO₃ (1.27 g. 9.15 mmol). The mixture was then stirred at 25° C., for 2 h. On completion, the reaction mixture was quenched with H₂O (7 mL), and then extracted with EtOAc (7 mL×3). The combined organic layers were washed with brine (7 mL×3), dried over Na₂SO₄, filtered and concentrated under reduced pressure to give a residue. The residue was purified by column chromatography (SiO₂, Petroleum ether/Ethyl acetate=99/1 to 4/1) to give the title compound (390 mg, 72% yield) as a white solid. ¹H NMR (400 MHZ, DMSO-d₆) δ=7.36 (s, 2H), 7.31-7.28 (m, 2H), 6.01 (d, J=2.4 Hz, 1H), 5.54 (d, J=2.0 Hz, 1H), 4.08 (s, 1H), 2.35 (br s, 2H), 1.00-0.98 (m, 2H), 0.93 (t, J=2.8 Hz, 2H).

### Tert-butyl (3-methoxy-1-methyl-1H-indazol-5-yl)methanamine (Intermediate IN)

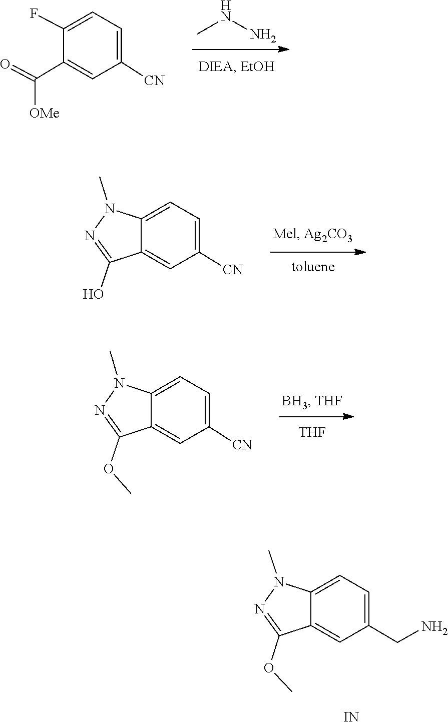

Step 1—3-Hydroxy-1-methyl-1H-indazole-5-carbonitrile. To a solution of methyl 5-cyano-2-fluoro-benzoate (1.00 g, 5.58 mmol, CAS #337362-21-7), methylhydrazine: sulfuric acid (2.41 g, 16.7 mmol, CAS #624-80-6) in EtOH (10 mL) was added DIEA (3.61 g, 27.9 mmol, 4.86 mL). The mixture was stirred at 25° C., for 2 h. On completion, the mixture was concentrated in vacuo then water (20 mL) was added and the mixture was filtered to give a residue. The filtrate was then extracted with EtOAc mL (20 mL×3). The combined organic layer was then with brine (30 mL×3), dried over anhydrous Na₂SO₄, and concentrated under reduced pressure to give the title compound (900 mg, 93% yield) as a white solid. LC-MS (ESI⁺) m/z 174.0 (M+H)⁺: ¹H NMR (400 MHZ, DMSO-d₆) δ=11.21 (br s, 1H), 8.16 (s, 1H), 7.62 (dd, J=1.2, 2.1 Hz, 2H), 3.83 (s, 3H).

Step 2—3-Methoxy-1-methyl-1H-indazole-5-carbonitrile. 3-Hydroxy-1-methyl-indazole-5-carbonitrile (800 mg, 4.62 mmol), iodomethane (6.56 g, 46.2 mmol, 2.88 mL) and Ag₂CO₃ (3.82 g, 13.8 mmol, 628 uL) were taken up in a microwave tube in toluene (8 mL). The sealed tube was heated at 60° C. for 2 h under microwave. On completion, the mixture was poured into H₂O (3 mL), then extracted with EtOAc mL (5 mL×3). The combined organic layer was washed with brine (5 mL), dried over anhydrous Na₂SO₄, filtered and concentrated under reduced pressure to give a residue. The residue was purified by column chromatography (SiO₂, Petroleum ether:Ethyl acetate=10:1 to Petroleum ether:Ethyl acetate=3:1) to give the title compound (600 mg, 69% yield) as a white solid. LC-MS (ESI⁺) m/z 187.8 (M+H)⁺: 1H NMR (400 MHZ, DMSO-d₆) δ=8.20 (d, J=1.0 Hz, 1H), 7.69 (t, J=1.2 Hz, 2H), 7.88-6.01 (m, 1H), 4.02 (s, 3H), 3.91 (s, 3H).

Step 3—Tert-butyl (3-methoxy-1-methyl-1H-indazol-5-yl)methanamine. To a solution of 3-methoxy-1-methyl-indazole-5-carbonitrile (600 mg, 3.21 mmol) in THF (10 mL) was added BH₃. THF (1 M, 11.2 mL) at 0° C. The mixture was stirred at 50° C., for 2 h. On completion, the reaction mixture was quenched by addition 1 M HCl until PH=4, and then addition NaOH aqueous solution until PH=10 and extracted with DCM 6 ml (2 ml×3). The combined organic layers were washed with brine 6 ml (3 ml×2), dried over Na₂SO₄, filtered and concentrated under reduced pressure to give a residue. To give the title compound (200 mg, crude) as a white solid. LC-MS (ESI⁺) m/z 175.2 (M+H)⁺: ¹H NMR (400 MHZ, DMSO-d₆) ¿=7.62 (s, 1H), 7.51-7.46 (m, 1H), 7.43-7.40 (m, 1H), 4.62 (s, 2H), 3.99 (s, 3H), 3.96 (s, 2H), 3.84 (s, 3H).

### (1,3-Dimethyl-1H-indazol-5-yl)methanamine (Intermediate IO)

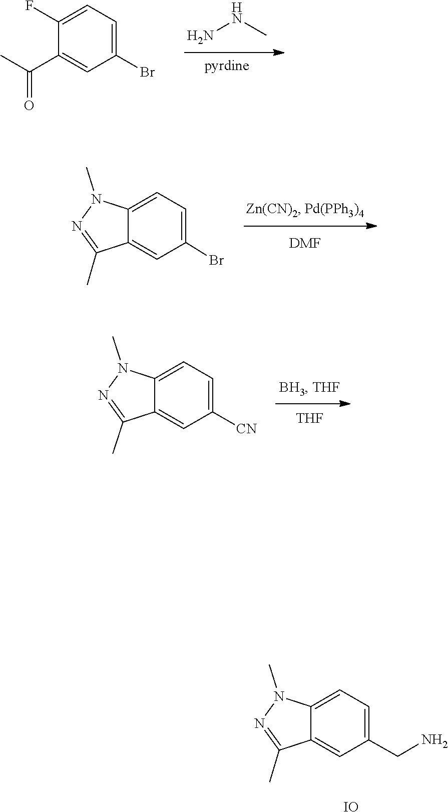

Step 1—5-Bromo-1,3-dimethyl-1H-indazole. To a solution of 1-(5-bromo-2-fluorophenyl)ethanone (500 mg, 2.30 mmol, CAS #198477-89-3) and methylhydrazine sulfuric acid (996 mg, 6.91 mmol, CAS #302-15-8) in pyridine (2.50 mL). The mixture was stirred at 100° C., for 12 hr. On completion, the reaction mixture was quenched with H₂O (5 mL), and then extracted with DCM (15 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=9/1) to afford the title compound (264 mg, 46% yield) as a yellow solid. ¹H NMR (400 MHZ, DMSO-d₆) δ=7.88 (d, J=1.2 Hz, 1H), 7.65 (d, J=8.4 Hz, 1H), 7.21 (dd, J=1.6, 8.5 Hz, 1H), 3.93 (s, 3H), 2.46 (s, 3H).

Step 2—1,3-Dimethyl-1H-indazole-5-carbonitrile. To a solution of 5-bromo-1,3-dimethyl-1H-indazole (264 mg, 1.17 mmol) in DMF (3.5 mL) was added Pd(PPh₃)⁺ (136 mg, 117 umol) and Zn(CN)₂ (207 mg, 1.76 mmol, 112 uL). The mixture was stirred at 100° C., for 12 hr. On completion, the reaction mixture was quenched with H₂O (10 mL), and extracted with EtOAc (15 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=91/9) to afford the title compound (157 mg, 77.4% yield) as a white solid. ¹H NMR (400 MHz, DMSO-d₆) δ=8.28 (s, 1H), 7.91 (d, J=8.4 Hz, 1H), 7.41 (dd, J=0.8, 8.3 Hz, 1H), 4.02 (s, 3H), 2.51 (br s, 3H).

Step 3—(1,3-Dimethyl-1H-indazol-5-yl)methanamine. To a solution of 1,3-dimethyl-1H-indazole-5-carbonitrile (157 mg, 917.07 umol) in THF (2.5 mL) was added BH₃. THF (1 M, 3.21 mL). The mixture was stirred at 0° C.˜50° C., for 2 hr. On completion, the reaction mixture was quenched with 1 M HCl until the pH=4. Then NaOH aqueous solution was added until the pH=10 and the mixture was extracted with DCM 15 ml (5 mL×3). The combined organic layers were washed with brine (3 mL×2), dried over Na₂SO₄, filtered and concentrated under reduced pressure to give a residue. The crude product was purified by reversed-phase HPLC (ACN/0.1% HCl=5%) to afford the title compound (91 mg, 56% yield) as a white solid. LC-MS (ESI⁺) m/z 176.3 (M+H)⁺.

### 1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)azetidine-3-carboxylic (Intermediate IP)

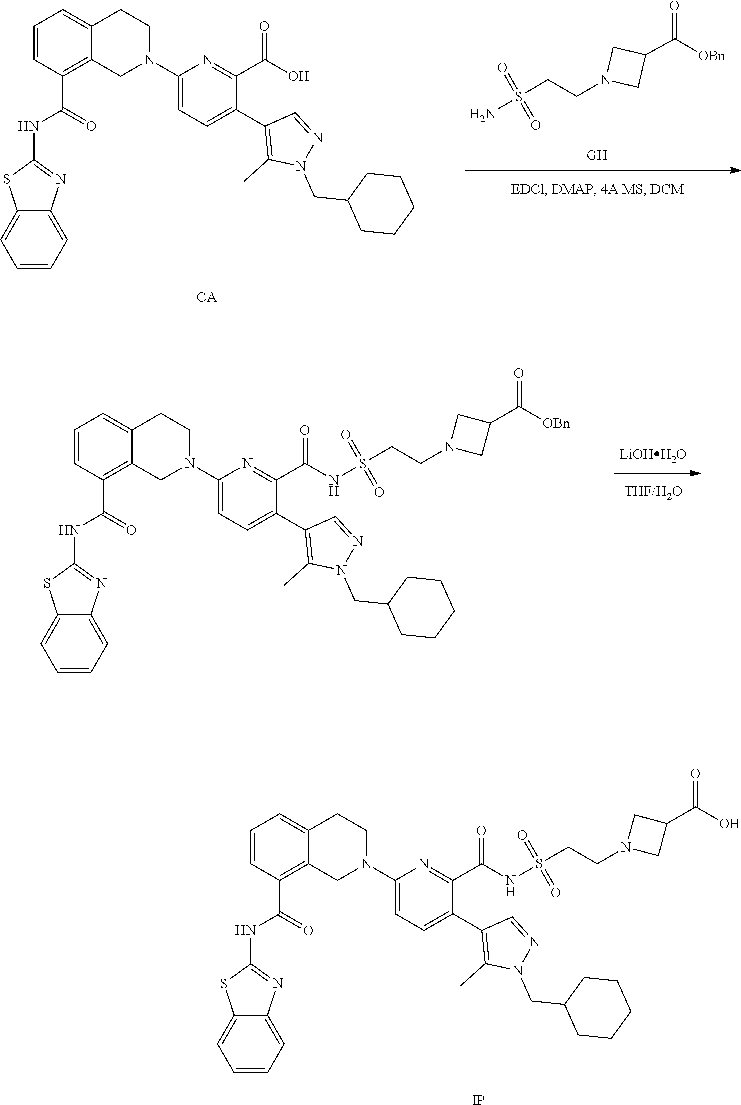 [SMILES: Cc1c(-c2ccc(N3CCc4cccc(C(=O)Nc5nc6ccccc6s5)c4C3)nc2C(=O)O)cnn1CC1CCCCC1 ; NS(=O)(=O)CCN1CC(C(=O)OCc2ccccc2)C1 ; Cc1c(-c2ccc(N3CCc4cccc(C(=O)Nc5nc6ccccc6s5)c4C3)nc2C(=O)NS(=O)(=O)CCN2CC(C(=O)OCc3ccccc3)C2)cnn1CC1CCCCC1 ; "LiOH·H2O" ; Cc1c(-c2ccc(N3CCc4cccc(C(=O)Nc5nc6ccccc6s5)c4C3)nc2C(=O)NS(=O)(=O)CCN2CC(C(=O)O)C2)cnn1CC1CCCCC1 ; "IP"]

Step 1—Benzyl 1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)azetidine-3-carboxylate. To a solution of benzyl 1-(2-sulfamoylethyl)azetidine-3-carboxylate (740 mg, 2.48 mmol, Intermediate GH) and 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (1.50 g, 2.48 mmol, Intermediate CA) in DCM (10 mL) was added EDCI (1.43 g, 7.44 mmol) DMAP (1.21 g, 9.92 mmol) and 4 Å molecular sieves (500 mg). The mixture was stirred at 25° C., for 2 hrs. On completion, the reaction mixture was diluted with H₂O (60 mL) and extracted with EA (75 mL×2). The combined organic layers were dried over Na₂SO₄, filtered and concentrated under reduced pressure to give a residue. The residue was purified by column chromatography (SiO₂, Petroleum ether/Ethyl acetate=1/1 to 0/1, DCM:MeOH=10:1) to give a title compound (2.5 g) as a yellow solid. LC-MS (ESI⁺) m/z 887.5 (M+H)+.

Step 2—1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)azetidine-3-carboxylic acid. To a solution of benzyl 1-[2-[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]ethyl]azetidine-3-carboxylate (2.5 g, 1.69 mmol, 60% purity) in THF (2 mL) and H₂O (2 mL) was added LiOH·H₂O (354 mg, 8.45 mmol). The mixture was stirred at 25° C., for 2 hours. On completion, the reaction mixture pH value of the solution to 6-7 by NH₄Cl, then diluted with H₂O (150 mL) and extracted with EA (100 mL×3). The combined organic layers were dried over Na₂SO₄, filtered and concentrated under reduced pressure to give a residue. The residue was purified by prep-HPLC (FA condition) to give the title compound (500 mg, 35% yield) as a yellow solid. LC-MS (ESI⁺) m/z 797.6 (M+H)⁺.

### Ethyl 1-(2-cyano-4-sulfamoylphenyl)piperidine-4-carboxylate (Intermediate IQ)

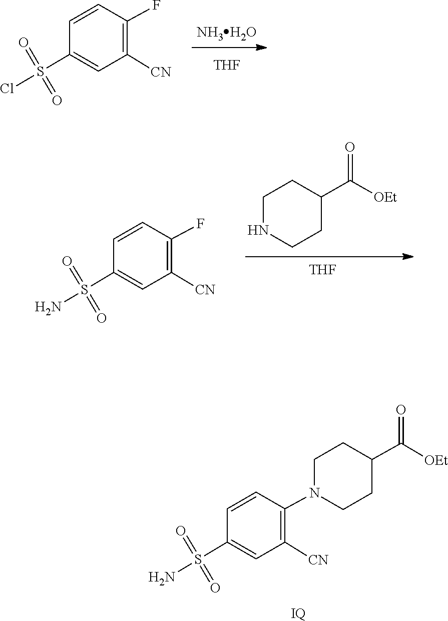

Step 1—3-Cyano-4-fluorobenzenesulfonamide. To a solution of 3-cyano-4-fluorobenzene-1-sulfonyl chloride (1.00 g, 4.55 mmol, CAS #351003-23-1) in THF (6 mL) was added NH₃·H₂O (6.38 g, 45.5 mmol, 7.01 mL, 25% solution) at 0° C. The mixture was then stirred at 0-25° C., for 1 h. On completion, the reaction was poured into water (5 mL) and extracted with EtOAc (10 mL×3). The combined organic phase is washed with brine (5 mL×2), and dried over sodium sulfate, filtered and concentrated to give a residue. The residue was purified by column chromatography (SiO₂, Petroleum ether/Ethyl acetate=0:1) to give the title compound (800 mg, 88% yield) as a brown solid. ¹H NMR (400 MHZ, DMSO-d₆) δ=8.32 (dd, J=2.0, 6.0 Hz, 1H), 8.19 (ddd, J=2.4, 5.4, 8.4 Hz, 1H), 7.76 (t, J=8.0 Hz, 1H), 7.63 (s, 2H).

Step 2—Ethyl 1-(2-cyano-4-sulfamoylphenyl)piperidine-4-carboxylate. To a solution of 3-cyano-4-fluorobenzenesulfonamide (350 mg, 1.75 mmol) and ethyl piperidine-4-carboxylate (412 mg, 2.62 mmol, 404 uL, CAS #1126 Sep. 6) in THF (4 mL). The sealed tube was heated at 100° C., for 0.7 h under microwave. On completion, the reaction mixture was concentrated under reduced pressure to give a residue. The residue was purified by column chromatography (SiO₂, Petroleum ether/Ethyl acetate=0:1) to give the title compound (350 mg) as a brown solid. LC-MS (ESI⁺) m/z 338.0 (M+H)⁺. ¹H NMR (400 MHZ, DMSO-d₆) δ=7.99 (d, J=2.4 Hz, 1H), 7.90 (dd, J=2.4, 9.2 Hz, 1H), 7.76 (t, J=8.8 Hz, 1H), 7.39 (br s, 2H), 4.10 (q, J=7.6 Hz, 2H), 3.65 (br d, J=12.4 Hz, 2H), 3.09-2.98 (m, 2H), 2.62-2.58 (m, 1H), 1.98 (br dd, J=3.2, 13.2 Hz, 2H), 1.79-1.66 (m, 2H), 1.20 (t, J=6.0 Hz, 3H).

### 1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)-2-cyanophenyl)piperidine-4-carboxylic acid (Intermediate IR)

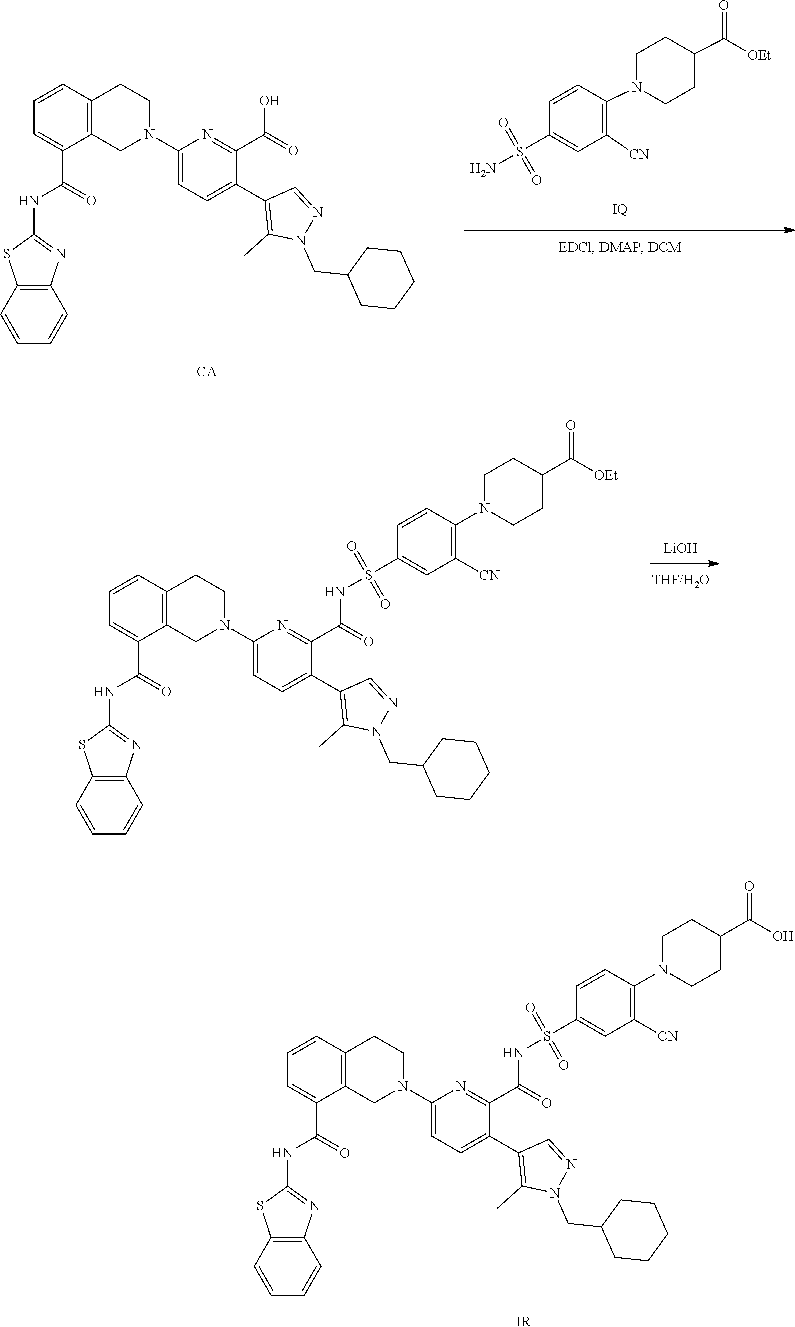

Step 1—Ethyl1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)-2-cyanophenyl)piperidine-4-carboxylate. To a solution of ethyl 1-(2-cyano-4-sulfamoylphenyl)piperidine-4-carboxylate (270 mg, 800 umol, Intermediate IQ), 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (405 mg, 667 umol, Intermediate CA) in DCM (3 mL) was added EDCI (320 mg, 1.67 mmol) and DMAP (163 mg, 1.33 mmol). The mixture was stirred at 25° C., for 12 h. On completion, the reaction was poured into water (5 mL) and extracted with EtOAc (10 mL×3). The combined organic phase was washed with brine (5 mL×2), and dried over sodium sulfate, filtered and concentrated to give a residue. The residue was purified by column chromatography (SiO₂, Petroleum ether/Ethyl acetate=5/1 to 1:1) to give the title compound (170 mg, 28% yield) as a yellow solid. LC-MS (ESI⁺) m/z=926.0 (M+H)⁺.

Step 2—1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)-2-cyanophenyl)piperidine-4-carboxylic acid To a solution of ethyl 1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)-2-cyanophenyl)piperidine-4-carboxylate (170 mg, 184 mol) in THF (2 mL) and H₂O (2 mL) was added LiOH·H₂O (38.5 mg, 918 umol). The mixture was stirred at 25° C., for 2 h. On completion, the pH of the reaction mixture was adjusted to 6˜ 5 by addition 2 M HCl, then the mixture was extracted with DCM (5 mL×5). The organic phase was separated, dried over sodium sulfate, filtered and concentrated under reduced pressure to give title compound (170 mg) as yellow solid. LC-MS (ESI⁺) m/z 898.8 (M+H)⁺.

### Ethyl 1-(2-sulfamoylethyl)piperidine-4-carboxylate (Intermediate IS)

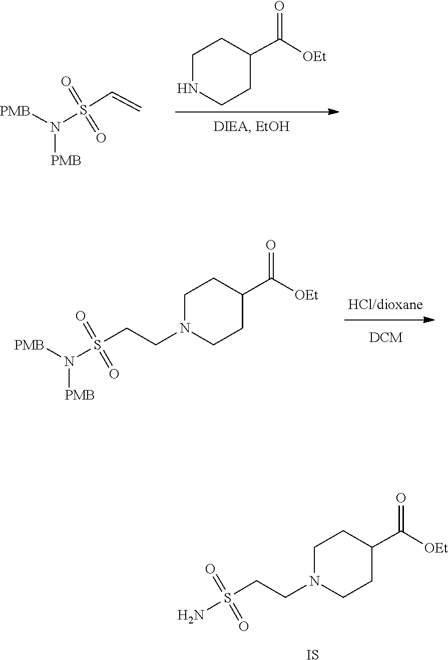

Step 1—Ethyl 1-(2-(N,N-bis(4-methoxybenzyl)sulfamoyl)ethyl)piperidine-4-carboxylate. To a solution of N, N, N-bis(4-methoxybenzyl)ethenesulfonamide (0.5 g, 1.44 mmol, CAS #133777-96-5) in EtOH (5 mL) was added DIEA (558 mg, 4.32 mmol) and ethyl piperidine-4-carboxylate (452 mg, 2.88 mmol, CAS #1126 Sep. 6). The mixture was stirred at 60° C., for 2 hrs. On completion, the reaction mixture was concentrated under reduced pressure to give a residue. The residue was purified by column chromatography (SiO₂, Petroleum ether/Ethyl acetate=10/1 to 0/1) to give the title compound (0.5 g, 68% yield) as a white solid. LC-MS (ESI⁺) m/z 505.8 (M+H)⁺.

Step 2—Ethyl 1-(2-sulfamoylethyl)piperidine-4-carboxylate. To a solution of ethyl 1-(2-(N, N-bis(4-methoxybenzyl)sulfamoyl)ethyl)piperidine-4-carboxylate (0.5 g, 990 umol) in DCM (10 mL) was added TFA (7.70 g, 67.5 mmol). The mixture was stirred at 40° C., for 36 hrs. On completion, the pH of reaction mixture was adjusted to weak basic condition (pH=8-9) with aq. NH₃ and then extracted with EA (15 mL×3). The combined organic layers were dried over Na₂SO₄ filtered and the filtrate was concentrated under reduced pressure to give a residue. The residue was purified by column chromatography (SiO₂, Petroleum ether/Ethyl acetate=10/1 to 0/1) to give the title compound (0.2 g, 76% yield) as a white solid. ¹H NMR (400 MHZ, DMSO-d₆) δ=6.75 (s, 2H), 4.08-4.02 (m, 2H), 3.15-3.11 (m, 2H), 2.78-2.66 (m, 4H), 2.03-2.02 (m, 2H), 1.77-1.76 (m, 2H), 1.55-1.54 (s, 1H), 1.17 (q, J=7.2 Hz, 3H).

### 1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)piperidine-4-carboxylic acid (Intermediate IT)

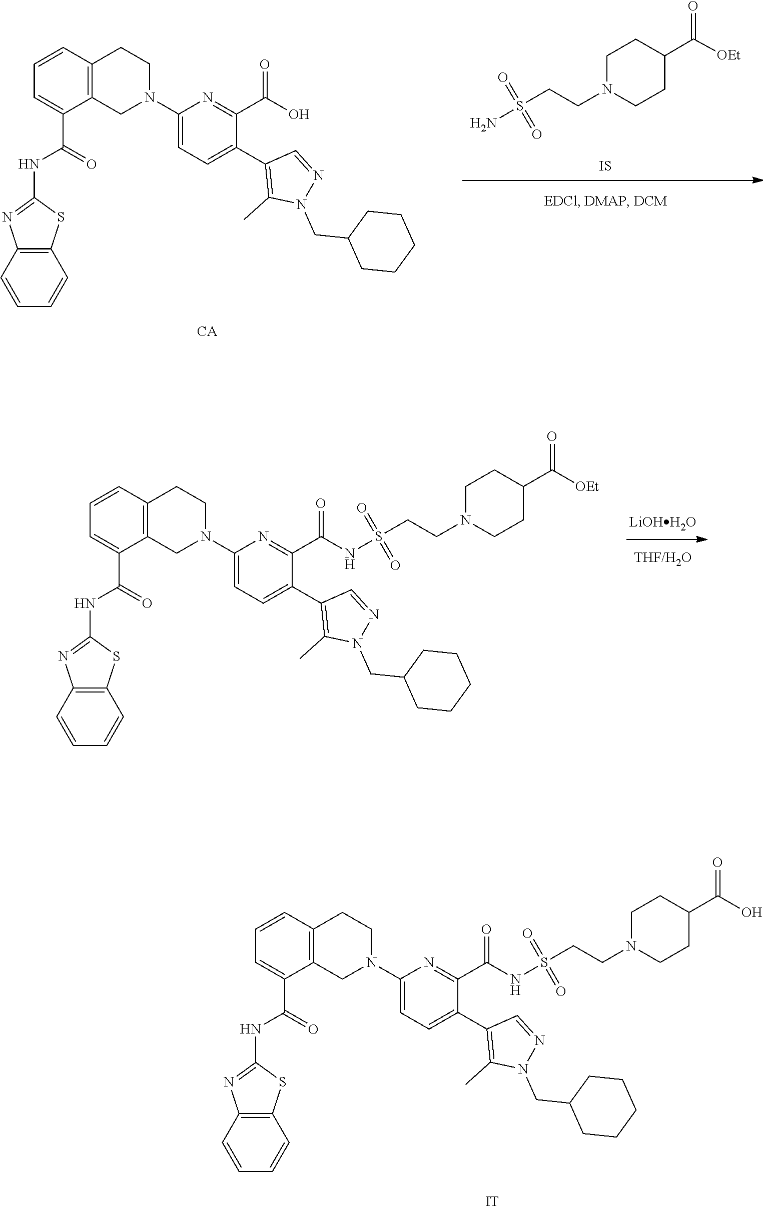

Step 1—Ethyl 1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)piperidine-4-carboxylate. To a solution of 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid (260 mg, 428 umol, Intermediate CA) and ethyl 1-(2-sulfamoylethyl)piperidine-4-carboxylate (169 mg, 642, umol, Intermediate IS) in DCM (4 mL) was added EDCI (246 mg, 1.29 mmol) and DMAP (209 mg, 1.71 mmol). The mixture was then stirred at 40° C., for 3 hrs. On completion, the reaction mixture was diluted with H₂O (15 mL) and extracted with EA (20 mL×3). The combined organic layers were dried over Na₂SO₄ filtered and the filtrate was concentrated under reduced pressure to give a residue. The residue was purified by column chromatography (SiO₂, DCM:MeOH=10:1) to give the title compound (220 mg, 60% yield) as a white solid. LC-MS (ESI⁺) m/z 853.7 (M+H)⁺.

Step 2—1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)piperidine-4-carboxylic acid. To a solution of ethyl 1-(2-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)ethyl)piperidine-4-carboxylate (0.2 g, 234 umol) in THF (2 mL) and H₂O (2 mL) was added LiOH—H₂O (98.3 mg, 2.34 mmol). The mixture was stirred at 25° C., for 12 hrs. On completion, the pH of reaction mixture was adjust to weak acid condition (pH=3-4) with HCl (1 M) and then extracted with EA (15 mL×3). The combined organic layers were dried over Na₂SO₄ filtered and concentrated under reduced pressure to give the title compound (150 mg) as a white solid. LC-MS (ESI⁺) m/z 825.1 (M+H)⁺.

### 1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl) propyl)piperidine-4-carboxylic acid (Intermediate IU)

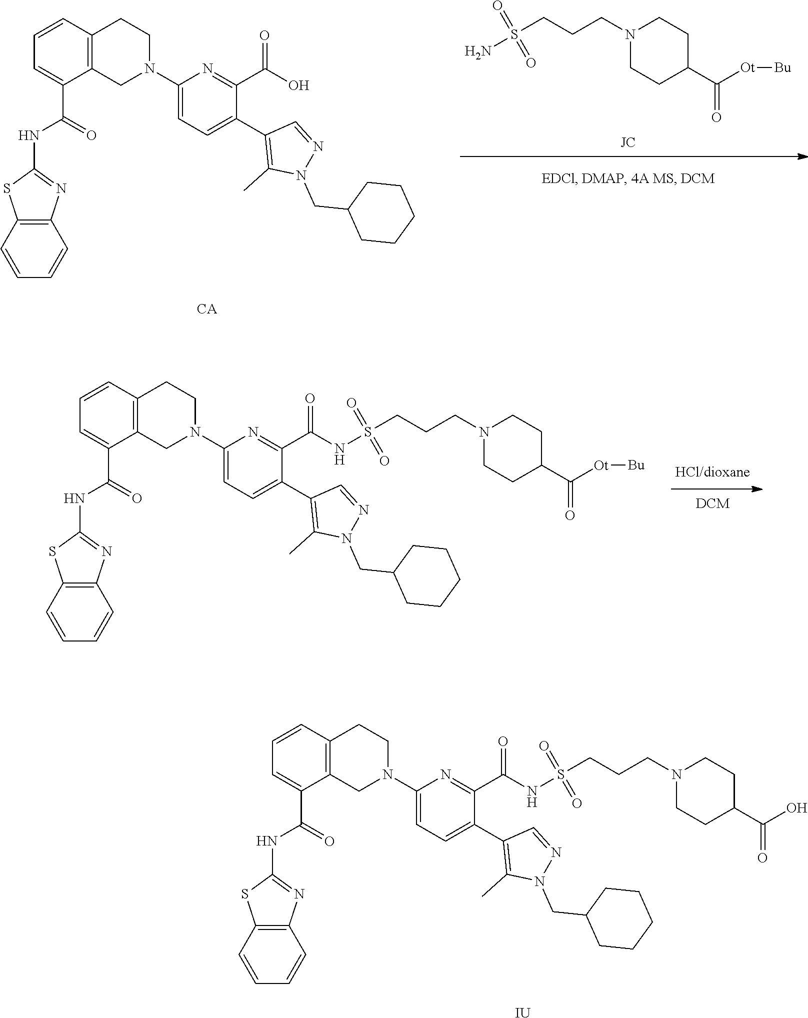

Step 1—Tert-butyl1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl) propyl)piperidine-4-carboxylate. To a solution of tert-butyl 1-(3-sulfamoylpropyl)piperidine-4-carboxylate (303 mg, 989 umol, Intermediate JC), 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (300 mg, 494 umol, Intermediate CA) in DCM (5 mL) was added EDCI (237 mg, 1.24 mmol), DMAP (181 mg, 1.48 mmol) and 4 Å molecular sieves (300 mg). The mixture was stirred at 25° C., for 12 h. On completion, the reaction was poured into water (5 mL) and extracted with DCM (10 mL×3). The combined organic phase is washed with brine (5 mL×2), dried over sodium sulfate, filtered and concentrated to give a residue. The crude product was purified by reversed-phase HPLC (0.1% FA condition) to give the title compound (130 mg, 29% yield) as a yellow solid. LC-MS (ESI⁺) m/z 895.1 (M+H)⁺.

Step 2—1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl) propyl)piperidine-4-carboxylic acid. To a solution of tert-butyl 1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl) propyl)piperidine-4-carboxylate (130 mg, 145 umol) in DCM (2 mL) was added HCl/dioxane (4 M, 2 mL). The mixture was stirred at 25° C., for 2 h. On completion, the reaction mixture was concentrated under reduced pressure to give the title compound (150 mg) as a white solid. LC-MS (ESI⁺) m/z 839.4 (M+H)⁺.

### 4-(1-Aminoethyl)-3-hydroxybenzonitrile (Intermediate IV)

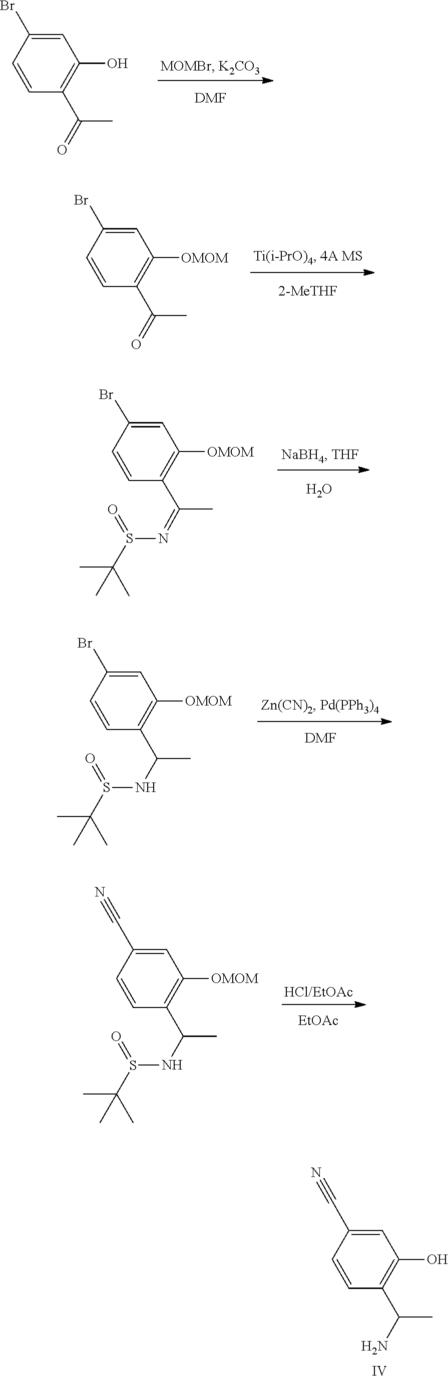

Step 1—1-(4-Bromo-2-(methoxymethoxy)phenyl)ethenone. To a solution of 1-(4-bromo-2-hydroxyphenyl)ethanone (10.0 g, 46.5 mmol, CAS #30186-18-6) and K₂CO; (19.3 g, 140 mmol) in DMF (90 mL) was added bromo(methoxy)methane (11.6 g, 93.0 mmol, 7.60 mL, CAS #13057-17-5) at 0° C. under N₂. The mixture was stirred at 0-25° C., for 12 h. On completion, the mixture was diluted with water (200 mL), and extracted with EtOAc (300 mL×3). The combined organic layers were washed with brine (80 mL×5), dried over Na₂SO₄ and evaporated. The residue was purified by column chromatography (SiO₂, Petroleum ether/Ethyl acetate=20/1 to 10/1) to afford the title compound (11.0 g, 91% yield) as a colorless oil. ¹H NMR (400 MHZ, DMSO-d₆) δ=7.53 (d, J=8.4 Hz, 1H), 7.42 (d, J=1.6 Hz, 1H), 7.28 (dd, J=1.6, 8.4 Hz, 1H), 5.37 (s, 2H), 3.43 (s, 3H), 2.55 (s, 3H).

Step 2—(Z)-N-(1-(4-bromo-2-(methoxymethoxy)phenyl)ethylidene)-2-methylpropane-2-sulfinamide. To a solution of 1-(4-bromo-2-(methoxymethoxy)phenyl)ethanone (11.0 g, 42.5 mmol), 2-methylpropane-2-sulfinamide (15.4 g, 127 mmol) and 4A molecular sieves (11.0 g) in 2-methyltetrahydrofuran (220 mL) was added Ti (i-PrO)⁺ (36.2 g, 127 mmol, 37.6 mL) at 0° C., under N₂. The mixture was heated to 90° C., for 12 h. On completion, the reaction mixture was diluted with EtOAc (800 mL), H₂O (100 mL) and filtered, evaporated. The mixture was diluted with water (100 mL), and extracted with EtOAc (200 mL×3). The combined organic layers were washed with brine (80 mL×3), dried over Na₂SO₄ and evaporated. The residue was purified by column chromatography (SiO₂, Petroleum ether/Ethyl acetate=3/1 to 2/1) to afford the title compound (3.50 g, 23% yield) as a yellow oil.

Step 3—N-(1-(4-bromo-2-(methoxymethoxy)phenyl)ethyl)-2-methylpropane-2-sulfinamide. To a solution of (Z)-N-(1-(4-bromo-2-(methoxymethoxy)phenyl)ethylidene)-2-methylpropane-2-sulfinamide (3.50 g, 9.66 mmol) in THF (34.3 mL) and H₂O (0.7 mL) was added NaBH₄ (1.83 g, 48.3 mmol) at −50° C., under N₂. The mixture was stirred at 25° C., for 12 h. On completion, the reaction mixture was quenched with H₂O (30 mL), and extracted with EtOAc (40 mL×3). The combined organic layers were washed with brine (10 mL×3), dried over Na₂SO₄ and evaporated. The residue was purified by column chromatography (SiO₂, Petroleum ether/Ethyl acetate=5/1 to 3/1) to afford the title compound (3.20 g, 91% yield) as a yellow solid. LC-MS (ESI⁺) m/z 365.9 (M+H)⁺.

Step 4—N-(1-(4-cyano-2-(methoxymethoxy)phenyl)ethyl)-2-methylpropane-2-sulfinamide. To a solution of N-(1-(4-bromo-2-(methoxymethoxy)phenyl)ethyl)-2-methylpropane-2-sulfinamide (3.10 g, 8.51 mmol) in DMF (31 mL) was added Pd(PPh₃)⁺ (983 mg, 851 umol) and Zn(CN) 2 (1.50 g, 12.8 mmol, 810 uL) under N₂. The mixture was stirred at 100° C., for 12 h. On completion, the reaction mixture was quenched with water (50 mL) and extracted with EtOAc (80 mL×3). The combined organic layers were washed with brine (20 mL×5), dried over Na₂SO₄ and evaporated. The residue was purified by column chromatography (SiO₂, Petroleum ether/Ethyl acetate=4/1 to 3/1) to afford the title compound (2.65 g) as a yellow oil. LC-MS (ESI⁺) m/z 310.9 (M+H)⁺.

Step 5—4-(1-aminoethyl)-3-hydroxybenzonitrile. To a solution of N-(1-(4-cyano-2-(methoxymethoxy)phenyl)ethyl)-2-methylpropane-2-sulfinamide (500 mg, 1.61 mmol) in EtOAc (3 mL) was added HCl/EtOAc (4 M, 5.00 mL). The mixture was stirred at 25° C., for 1 h. On completion, the reaction mixture was evaporated to afford the title compound (320 mg, HCl salt) as a white solid. 1-(3-methoxy-1-methyl-1H-indazol-5-yl)ethan-1-amine (Intermediate IW)

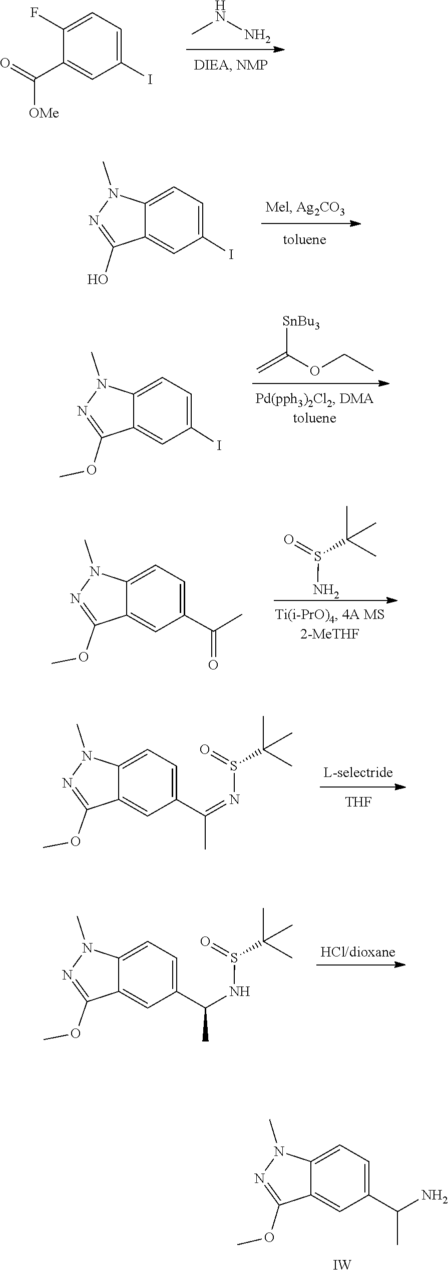

Step 1—5-Iodo-1-methyl-1H-indazol-3-ol. To a solution of methyl 5-bromo-2-fluoro-benzoate (3 g, 12.8 mmol, CAS #625471-27-4) and methylhydrazine (2.54 g, 22.1 mmol, 2.90 mL, 40% solution) in NMP (30 mL) was added DIEA (1.75 g, 13.5 mmol, 2.35 mL) at 20° C. Then the reaction was stirred at 140° C., for 6 h in a sealed tube. On completion, the reaction was poured into water (30 mL) and extracted with EtOAc (30 mL×2). The combined organic phase is washed with brine (20 mL×2), dried over Na₂SO₄, filtered and concentrated to give the title compound (1.30 g, 45% yield) as white solid.

Step 2—5-Iodo-3-methoxy-1-methyl-1H-indazole. To a solution of 5-bromo-1-methyl-indazol-3-ol (500 mg, 2.20 mmol) and Mel (3.13 g, 22.0 mmol, 1.37 mL) in toluene (9 mL) was added Ag₂CO₃ (1.82 g, 6.61 mmol, 299 uL) at 20° C., under nitrogen flow. Then the reaction was stirred in a microwave at 60° C., for 2 h. On completion, the reaction was filtered to get the filtrate then poured into water (10 mL) and extracted with EtOAc (20 mL×2). The combined organic phase was washed with brine (10 mL×2), dried over Na₂SO₄, filtered and concentrated to give a residue. The residue was purified by silica gel column chromagraphy (eluted with petroleum ether:ethyl acetate=100:1 to 100:30) to give the title compound (480 mg, 90% yield) as a yellow oil.

Step 3—1-(3-Methoxy-1-methyl-1H-indazol-5-yl)ethenone. To a solution of 5-iodo-3-methoxy-1-methyl-1H-indazole (430 mg, 1.78 mmol) and tributyl (1-ethoxyvinyl) stannane (0.76 g, 2.10 mmol, 710 uL, CAS #97674-02-7) in toluene (4.3 mL) was added Pd(PPh₃)₂Cl₂ (62.6 mg, 89.1 umol) under N₂, then the mixture was heated to 90° C., and stirred for 12 h. On completion, the reaction mixture was cooled to 25° C., filtered and washed with EtOAc (12 mL). The filtrate was evaporated and 5N HCl (3 mL) was added and the mixture was stirred at 25° C., for 1.5 h. On completion, the reaction mixture was quenched with water (5 mL) and the pH was adjusted to 8˜7 with sat. NaHCO₃. Then KF (500 mg) was added and the mixture was stirred for 1 h at 25° C. The solution was then extracted with EtOAc (10 mL×3) and the combined organic layers were washed with brine (3 mL×3), dried over Na₂SO₄ and evaporated. The residue was purified by column chromatography (SiO₂, Petroleum ether/Ethyl acetate=20/1 to 10/1) to give the title compound (300 mg, 82% yield) as yellow gum. ¹H NMR (400 MHz, DMSO-d₆) δ=8.28 (s, 1H), 7.94 (dd, J=1.2, 8.8 Hz, 1H), 7.55 (d, J=9.2 Hz, 1H), 4.03 (s, 3H), 3.89 (s, 3H), 2.60 (s, 3H).

Step 4—(R,Z)-N-(1-(3-methoxy-1-methyl-1H-indazol-5-yl)ethylidene)-2-methylpropane-2-sulfinamide. To a solution of 1-(3-methoxy-1-methyl-1H-indazol-5-yl)ethanone (300 mg, 1.47 mmol), (R)-2-methylpropane-2-sulfinamide (712 mg, 5.88 mmol, CAS #196929-78-9), and 4A molecular sieves (300 mg) in 2-MeTHF (3 mL) was added Ti (Oi-Pr)⁺ (1.25 g, 4.41 mmol, 1.30 mL) at 0° C., under N₂. Then the mixture was stirred at 100° C., for 12 h under N₂. On completion, the reaction mixture was quenched with aq. NH₄Cl (20 mL) at 20° C., and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (150 mg, 33.2% yield) as yellow solid. LC-MS (ESI⁺) m/z 308.0 (M+H)⁺.

Step 5 (R)-N-((S)-1-(3-methoxy-1-methyl-1H-indazol-5-yl)ethyl)-2-methylpropane-2-sulfinamide. To a solution of (R,Z)-N-(1-(3-methoxy-1-methyl-1H-indazol-5-yl)ethylidene)-2-methylpropane-2-sulfinamide (150 mg, 487 umol) in THF (2 mL) was added L-selectride (1 M, 731 uL) at 0° C., under N₂, then the mixture was stirred at 0-25° C., for 2 h under N₂ atmosphere. On completion, the reaction mixture was quenched with NH₄Cl 2 mL at 0° C., and then diluted with H₂O (3 mL) and extracted with EtOAc (5 mL×2). The combined organic layers were washed with aqueous NaCl (5 mL×2), dried over Na₂SO₄, filtered and concentrated under reduced pressure to give the title compound (150 mg) as yellow gum.

Step 6-1-(3-methoxy-1-methyl-1H-indazol-5-yl)ethanamine. To a solution of (R)-N-((S)-1-(3-methoxy-1-methyl-1H-indazol-5-yl)ethyl)-2-methylpropane-2-sulfinamide (150 mg, 484 umol) in dioxane (1 mL) was added HCl/dioxane (4 M, 1 mL), then the mixture was stirred at 25° C., for 0.5 h. On completion, the reaction mixture was concentrated in vacuo, and the residue was purified by prep-HPLC (0.1% FA condition) to give the title compound (100 mg) as yellow gum. LC-MS (ESI⁺) m/z 189.1 (M+H-17)⁺.

### 1-(1,3-dimethyl-1H-indazol-5-yl)ethanamine (Intermediate IX)

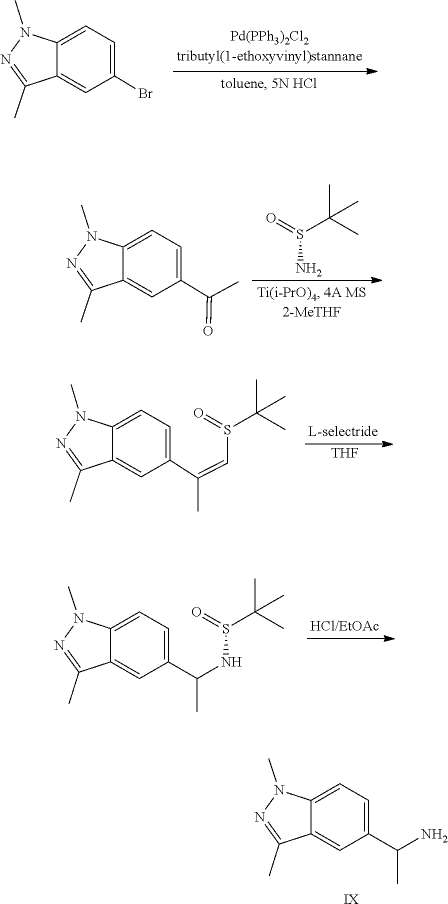

Step 1—1-(1,3-Dimethyl-1H-indazol-5-yl)ethenone. To a solution of 5-bromo-1,3-dimethyl-indazole (3.00 g, 13.3 mmol, synthesized via Step 1 of Intermediate IO) and tributyl (1-ethoxyvinyl) stannane (5.78 g, 16.0 mmol, 5.40 mL, CAS #97674-02-7) in toluene (30 mL) was added Pd(PPh₃)₂Cl₂ (468 mg, 666 umol) under N₂. The mixture was then heated to 90° C., and stirred for 12 h. On completion, the reaction mixture was cooled to 25° C., filtered and washed with EtOAc (20 mL). The filtrate was then evaporated and 5N HCl (20 mL) was added and the mixture was stirred at 25° C., for 1.5 h. On completion, the reaction mixture was quenched with water (5 mL) and the pH was adjusted to 8˜7 with sat. NaHCO₃, then extracted with EtOAc (40 mL×3). The combined organic layers were washed with brine (20 mL×3), dried over Na₂SO₄ and evaporated. The residue was purified by column chromatography (SiO₂, Petroleum ether/Ethyl acetate=5/1 to 3/1) to afford the title compound (2.20 g, 88% yield) as a yellow solid. ¹H NMR (400 MHZ, DMSO-d₆) δ=8.26 (s, 1H), 7.79 (d, J=8.4 Hz, 1H), 7.64 (dd, J=1.2, 8.4 Hz, 1H), 4.06 (s, 3H), 2.68 (s, 3H), 2.52-2.50 (m, 3H).

Step 2—(R,Z)-N-(1-(1,3-dimethyl-1H-indazol-5-yl)ethylidene)-2-methylpropane-2-sulfinamide. To a solution of 1-(1,3-dimethyl-1H-indazol-5-yl)ethanone (450 mg, 2.39 mmol), (R)-2-methylpropane-2-sulfinamide (869 mg, 7.17 mmol, CAS #196929-78-9) and 4A molecular sieves (500 mg) in 2-methyltetrahydrofuran (5 mL) was added Ti (i-PrO)⁺ (2.04 g, 7.17 mmol, 2.12 mL) at 0° C., under N₂. The mixture was then heated to 100° C., for 12 h. On completion, the reaction mixture was diluted with EtOAc (50 mL) and H₂O (10 mL) and filtered. The mixture was diluted with water (10 mL), extracted with EtOAc (20 mL×3). The combined organic layers were washed with brine (20 mL×3), dried over Na₂SO₄ and evaporated. The residue was purified by column chromatography (SiO₂, Petroleum ether/Ethyl acetate=3/1 to 2/1) to afford the title compound (900 mg, 27% yield) as a yellow solid. LC-MS (ESI⁺) m z 291.9 (M+H)⁺.

Step 3—(R)—N-(1-(1,3-dimethyl-1H-indazol-5-yl)ethyl)-2-methylpropane-2-sulfinamide. To a solution of (R,Z)-N-(1-(1,3-dimethyl-1H-indazol-5-yl)ethylidene)-2-methylpropane-2-sulfinamide (700 mg, 2.04 mmol) in THF (7 mL) was added L-selectride (1 M, 3.06 mL) at 0° C., under N₂. The mixture was stirred at 25° C., for 2 h. On completion, the reaction mixture was quenched with NH₄Cl (10 mL) at 0° C., and then diluted with H₂O (5 mL) and extracted with EtOAc (15 mL×3). The combined organic layers were washed with aqueous NaCl (5 mL×2), dried over Na₂SO₄, filtered and concentrated under reduced pressure to give the title compound (700 mg) as a yellow oil. LC-MS (ESI⁺) m/z 294.2 (M+H)⁺.

Step 4—1-(1,3-Dimethyl-1H-indazol-5-yl)ethanamine. To a solution of (R)—N-(1-(1,3-dimethyl-1H-indazol-5-yl)ethyl)-2-methylpropane-2-sulfinamide (700 mg, 1.91 mmol) in EtOAc (7 mL) was added HCl/EtOAc (4 M, 7.00 mL). The mixture was stirred at 25° C., for 1 h. On completion, the reaction mixture concentrated under reduced pressure to give the title compound (700 mg, HCl salt) as a white solid.

### (2S,4R)-1-((S)-2-amino-3,3-dimethylbutanoyl)-N-(4-ethynyl-2-(2-hydroxyethoxy)benzyl)-4-hydroxypyrrolidine-2-carboxamide (Intermediate IY)

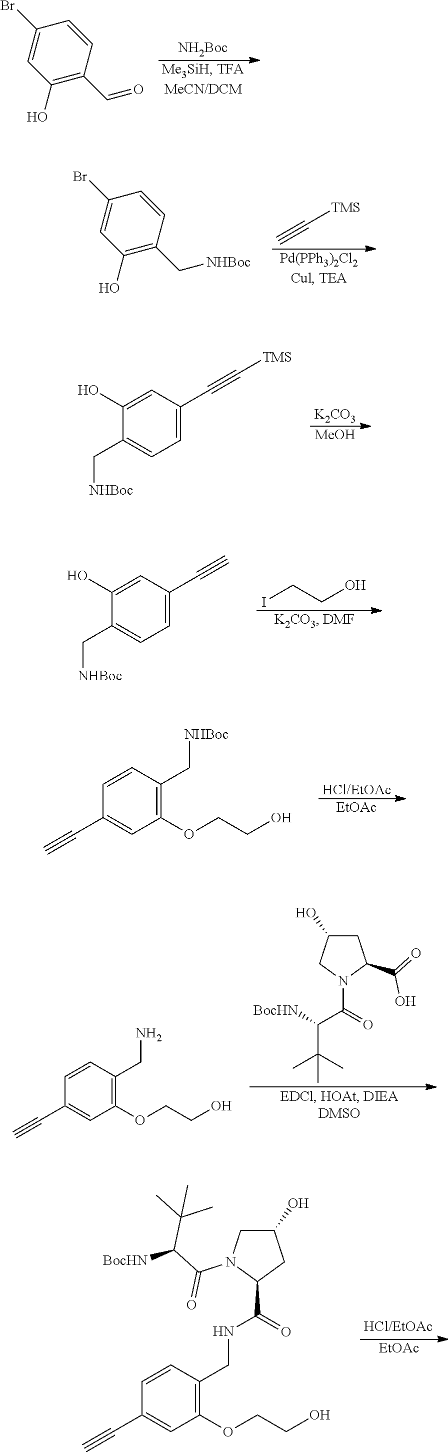

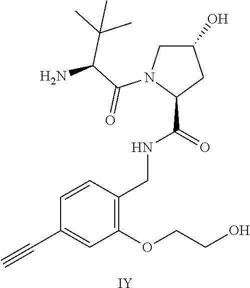

Step 1—Tert-butyl 4-bromo-2-hydroxybenzylcarbamate. To a solution of 4-bromo-2-hydroxy-benzaldehyde (30 g, 150 mmol) in DCM (100 mL) ACN (300 mL) was added Et₃SiH (52.06 g, 447.72 mmol, 71.51 mL) and TFA (34.03 g, 298.5 mmol, 22.10 mL) and tert-butyl carbamate (52.45 g, 447.7 mmol). The mixture was stirred at 25° C., for 12 hr. On completion, the reaction mixture was concentrated under reduced pressure to give a residue. The crude product was purified by MPLC (SiO₂, PE:EA=50:1 to 10:1) to give the title compound (58 g, 97% yield) as a white solid. LC-MS (ESI) m/z 326.1 (M+Na).

Step 2—Tert-butyl 2-hydroxy-4-((trimethylsilyl)ethynyl)benzylcarbamate. A mixture of tert-butyl N-[(4-bromo-2-hydroxy-phenyl)methyl]carbamate (35 g, 120 mmol), ethynyl (trimethyl)silane (91.0 g. 927 mmol. 128 mL), Pd(PPh₃)₂Cl₂ (4.07 g, 5.79 mmol), CuI (2.21 g, 11.6 mmol) in TEA (300 mL) was degassed and purged with N₂ three times. Then the mixture was stirred at 80° C., for 12 hours. The reaction mixture was quenched with sat. NH₄Cl (100 mL) at 25° C., and then diluted with ethyl acetate (100 mL) and extracted with ethyl acetate (150 mL×3). The combined organic layers were washed with sat. NaCl (300 mL×1), dried over Na₂SO₄, filtered and concentrated under reduced pressure to give a residue. The crude product was purified by MPLC (SiO₂, PE:EA=10/1 to 1/1) to give the title compound (12 g, 31% yield) as white solid: LC-MS (ESI) m/z 342.2 (M+Na).

Step 3—2-(Aminomethyl)-5-ethynylphenol. To a solution of tert-butyl N-∥2-hydroxy-4-(2-trimethylsilylethynyl)phenyl]methyl]carbamate (0.6 g, 2 mmol) in MeOH (7 mL) was added K₂CO₃ (519.13 mg. 3.76 mmol). The mixture was then stirred at 20° C., for 12 hr. On completion, the mixture was concentrated under reduce pressure to give the title compound (480 mg) as white solid. ¹H NMR (400 MHZ, DMSO-d₆) δ 9.76 (s, 1H), 7.24-7.15 (m, 1H), 7.08-6.99 (m, 1H), 6.98-6.91 (m, 1H), 6.90-6.83 (m, 1H), 4.11-3.99 (m, 3H), 1.39 (s, 9H).

Step 4—Tert-butyl 4-ethynyl-2-(2-hydroxyethoxy)benzylcarbamate. A mixture of tert-butyl 4-ethynyl-2-hydroxybenzylcarbamate (3 g, 12.13 mmol), 2-iodoethanol (3.80 g, 22.08 mmol, 1.73 mL), and K₂CO; (5.03 g, 36.4 mmol), in DMF (40 mL) was degassed and purged with N₂ three times. Then the mixture was stirred at 80° C., for 12 hr under N₂ atmosphere. On completion, the reaction mixture was cool down rt and diluted with water (100 mL) and extracted with EtOAc (20 mL×3). The combined organic layers were washed with brine (10 mL×3), dried over Na₂SO₄ and evaporated. The residue was purified by column chromatography (SiO₂, Petroleum ether/Ethyl acetate=10:1) to give the title compound (2.5 g. 63% yield) as a white solid. LC-MS (ESI) m/z 174.7 (M+H); ¹H NMR (400 MHZ, DMSO-d₆) δ=7.21 (br t, J=6.1 Hz, 1H), 7.12 (d, J=7.5 Hz, 1H), 7.07-7.02 (m, 2H), 4.87 (t, J=5.6 Hz, 1H), 4.15-4.10 (m, 3H), 4.01 (t, J=4.8 Hz, 2H), 3.72 (q. J=5.0 Hz, 2H), 2.89 (s, 1H), 2.73 (s, 1H), 1.39 (s, 9H), 0.95-0.71 (m, 1H).

Step 5—2-(2-(Aminomethyl)-5-ethynylphenoxy) ethanol. A mixture of tert-butyl 4-ethynyl-2-(2-hydroxyethoxy)benzylcarbamate (1.4 g, 2.77 mmol) and LiOH·H₂O (349.27 mg. 8.32 mmol) in MeOH (3 mL), THF (3 mL) and H₂O (3 mL) was degassed and purged with N₂ three times, and then the mixture was stirred at 25° C., for 16 hr under N₂ atmosphere. On completion, the reaction mixture was concentrated under vacuum and diluted with water (20 mL) and extracted with EtOAc (20 mL×3). The combined organic layers were washed with brine (20 mL×3), dried over Na₂SO₄ and evaporated to give the title compound (1 g, 70% yield) as a white solid. LC-MS (ESI⁺) m/z 174.7 (M+H)+.

Step 6-Tert-butyl ((S)-1-((2S,4R)-2-((4-ethynyl-2-(2-hydroxyethoxy)benzyl)carbamoyl)-4-hydroxypyrrolidin-1-yl)-3,3-dimethyl-1-oxobutan-2-yl) carbamate. A mixture of 2-(2-(aminomethyl)-5-ethynylphenoxy) ethanol (0.74 g, 3.87 mmol), (2S,4R)-1-((S)-2-((tert-butoxycarbonyl)amino)-3,3-dimethylbutanoyl)-4-hydroxypyrrolidine-2-carboxylic acid (1.33 g, 3.87 mmol), EDCI (1.63 g, 8.51 mmol), HOAt (1.16 g, 8.51 mmol, 1.19 mL) and DIEA (2.50 g, 19.35 mmol, 3.37 mL) in DMSO (5 mL) was degassed and purged with N₂ three times. Then the mixture was stirred at 25° C., for 2 hr under N₂ atmosphere. The crude product was purified by reversed-phase HPLC (ACN/0.1% FA=45%) to give the title compound (0.74 g, 36% yield) as a white solid. LC-MS (ESI⁺) m/z 418 (M+H)⁺.

Step 7-(2S,4R)-1-((S)-2-amino-3,3-dimethylbutanoyl)-N-(4-ethynyl-2-(2-hydroxyethoxy)benzyl)-4-hydroxypyrrolidine-2-carboxamide. A mixture of tert-butyl ((S)-1-((2S,4R)-2-((4-ethynyl-2-(2-hydroxyethoxy)benzyl)carbamoyl)-4-hydroxypyrrolidin-1-yl)-3,3-dimethyl-1-oxobutan-2-yl) carbamate (0.73 g, 1.41 mmol) and HCl/EtOAc (4 mL) in EtOAc (4 mL) was degassed and purged with N₂ three times, and then the mixture was stirred at 25° C., for 0.5 hr under N₂ atmosphere. On completion, the crude product was purified by reversed-phase HPLC (ACN/0.1% FA=35%) to give the title compound (120 mg, 17% yield) as a white solid. LC-MS (ESI⁺) m/z 417 (M+H)⁺.

### Ethyl 3-(1-sulfamoylpiperidin-4-yl) propiolate (Intermediate IZ)

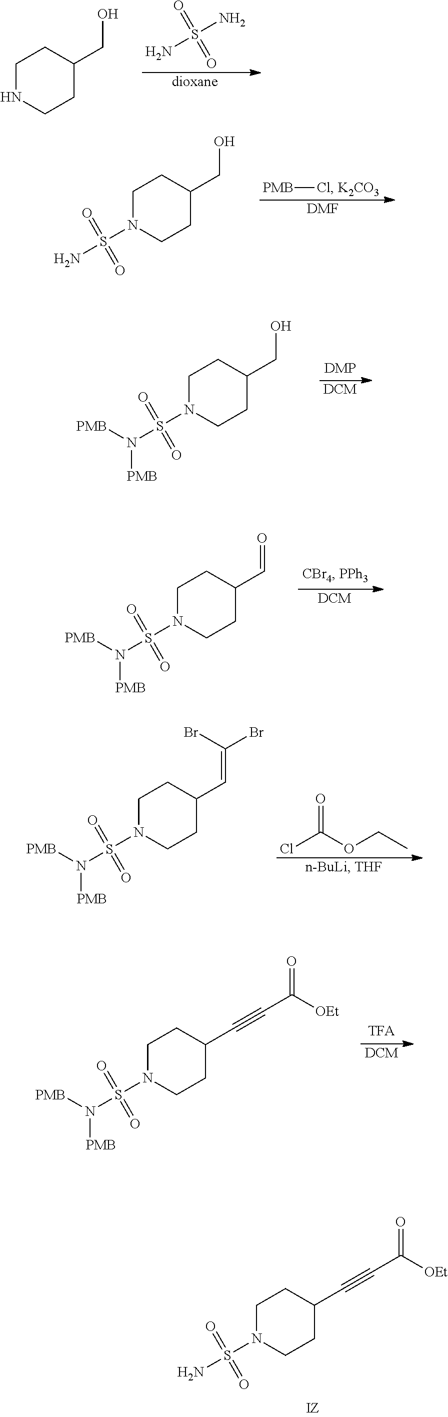

Step 1—4-(hydroxymethyl)piperidine-1-sulfonamide. A solution of sulfamide (4.59 g, 47.8 mmol. CAS #7803-58-9). 4-piperidylmethanol (5.00 g, 43.4 mmol. CAS #6457-49-4) in dioxane (75 mL) was degassed and purged with N₂ three times, and then the mixture was stirred at 105° C., for 12 h under N₂ atmosphere. On completion, the reaction mixture was dried over Na₂SO₄, filtered and concentrated under reduced pressure to give a residue. The residue was triturated with IM HCl (80 mL) at 25° C., for 5 min and filtered. Then the filter cake was dried in vacuo to give the title compound (4.74 g, 56% yield) as a white solid. ¹H NMR (400 MHZ. DMSO-d₆) ¿=3.92-3.89 (m, 1H), 3.56 (br s, 1H), 3.44 (br d, J=11.6 Hz. 2H), 3.26 (d, J=6.4 Hz, 2H), 2.52-2.43 (m, 2H), 1.72 (br d, J=11.2 Hz, 2H), 1.44-1.33 (m, 1H), 1.21-1.05 (m, 2H).

Step 2—4-(Hydroxymethyl)-N,N-bis(4-methoxybenzyl)piperidine-1-sulfonamide. To solution of 4-(hydroxymethyl)piperidine-1-sulfonamide (4.24 g, 21.8 mmol). PMB-CI (8.55 g, 54.6 mmol. 7.43 mL. CAS #824-94-2) in DMF (42 mL) at 0° C., was added K₂CO₃ (12.1 g, 87.3 mmol), then the mixture was stirred at 50° C., for 8 h. On completion, the reaction mixture was quenched with H₂O (100 mL), and then extracted with EtOAc (100 mL×3). The combined organic layers were washed with brine (50 mL×3), dried over Na₂SO₄, filtered and concentrated under reduced pressure to give a residue. The residue was purified by column chromatography (SiO₂. Petroleum ether/Ethyl acetate=3/1 to 1/1) to give the title compound (5.90 g, 62% yield) as a white solid. ¹H NMR (400 MHZ. DMSO-d₆) ¿=7.96 (s, 1H), 7.15 (d, J=8.8 Hz, 4H), 6.92-6.86 (m, 4H), 4.51 (t, J=5.2 Hz, 1H), 4.15 (s. 4H), 3.51 (br d, J=12.0 Hz, 2H). 3.24 (t, J=6.0 Hz, 2H), 2.89 (s, 2H), 2.74 (s, 1H), 2.66 (br d, J=2.4 Hz, 2H), 1.72-1.64 (m, 2H), 1.45 (br s. 1H), 1.14-1.02 (m, 2H).

Step 3—4-Formyl-N,N-bis(4-methoxybenzyl)piperidine-1-sulfonamide. To a solution of 4-(hydroxymethyl)-N,N-bis(4-methoxybenzyl)piperidine-1-sulfonamide (3.00 g, 6.90 mmol), dess-martin periodinane (4.39 g, 10.4 mmol) in DCM (60 mL) was degassed and purged with N₂ three times, and then the mixture was stirred at 25° C., for 1 h under N₂ atmosphere. On completion, the reaction mixture was quenched with H₂O (60 mL), and then extracted with DCM (80 mL×3). The combined organic layers were washed with brine (20 mL×3), dried over Na₂SO₄, filtered and concentrated under reduced pressure to give a residue. The residue was purified by column chromatography (SiO₂. Petroleum ether/Ethyl acetate=15/1 to 10/1) to give the title compound (2.08 g, 70% yield) as a white solid. LC-MS (ESI⁺) m/z 433.3 (M+H).

Step 4—4-(2,2-Dibromovinyl)-N,N-bis(4-methoxybenzyl)piperidine-1-sulfonamide. To a solution of 4-formyl-N,N-bis(4-methoxy benzyl)piperidine-1-sulfonamide (1.88 g, 4.35 mmol) in DCM (9.4 mL) was added CBr₄ (2.88 g, 8.70 mmol) and PPh: (4.56 g, 17.4 mmol) at 0° C. The mixture was stirred at 0° C., for 0.5 h. On completion, the reaction mixture was quenched with H₂O (10 mL), and then extracted with DCM (20 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=10/1 to 9/1) to give the title compound (2.00 g, 78% yield) as a white solid. ¹H NMR (400 MHZ, DMSO-d₆) δ=7.15 (d, J=8.4 Hz, 4H), 6.90 (d, J=8.4 Hz, 4H), 6.53 (d, J=8.8 Hz, 1H), 4.16 (s, 4H), 3.75 (s, 6H), 3.49 (br d, J=12.4 Hz, 2H), 2.82-2.72 (m, 2H), 2.40-2.28 (m, 1H), 1.67 (br d, J=10.8 Hz, 2H), 1.46-1.34 (m, 2H).

Step 5—Ethyl 3-(1-(N,N-bis(4-methoxybenzyl)sulfamoyl)piperidin-4-yl) propiolate. To a solution of 4-(2,2-dibromovinyl)-N,N-bis(4-methoxybenzyl)piperidine-1-sulfonamide (1.80 g, 3.06 mmol) in THF (36 mL) was added n-BuLi (2.5 M, 2.45 mL) at −78° C., under a nitrogen atmosphere, and the mixture was stirred at −78° C.-0° C., for 1 h. Then ethyl carbonochloridate (1.16 g, 10.7 mmol, CAS #541-41-3) was added at −78° C., and then the mixture was stirred at 25° C., for 0.5 h under N₂ atmosphere. On completion, the reaction mixture was quenched with HN₄Cl (60 mL), and then extracted with EtOAc (50 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=15/1 to 10/1) to give the title compound (1.22 g, 80% yield) as a white solid. ¹H NMR (400 MHZ, DMSO-d₆) δ=7.16 (d, J=8.8 Hz, 4H), 6.91 (d, J=8.8 Hz, 4H), 4.17 (t, J=3.6 Hz, 6H), 3.75 (s, 6H), 3.26 (br s, 2H), 2.95 (br s, 2H), 2.88-2.81 (m, 1H), 1.88-1.80 (m, 2H), 1.60-1.50 (m, 2H), 1.22 (t, J=7.2 Hz, 3H).

Step 6-Ethyl 3-(1-sulfamoylpiperidin-4-yl) propiolate. A solution of ethyl 3-(1-(N,N-bis(4-methoxy benzyl)sulfamoyl)piperidin-4-yl) propiolate (1.10 g, 2.20 mmol) and TFA (8.47 g, 74.3 mmol) in DCM (11 mL) was degassed and purged with N₂ three times. Then the mixture was stirred at 25° C., for 12 h under N₂ atmosphere. On completion, the reaction mixture was quenched with NaHCO; (50 mL), and then extracted with DCM (50 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3/1 to 1/1) to give the title compound (660 mg, 99% yield) as an orange solid. ¹H NMR (400 MHZ, DMSO-d₆) δ=6.78 (s, 2H), 4.15 (q, J=7.2 Hz, 2H), 4.03 (d, J=7.2 Hz, 1H), 3.75-3.53 (m, 2H), 3.23-3.16 (m, 2H), 2.85-2.76 (m, 3H), 1.99 (s, 1H), 1.89 (br d, J=2.8 Hz, 2H), 1.69-1.59 (m, 2H), 1.21 (t, J=7.2 Hz, 3H).

### 3-(1-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)piperidin-4-yl) propiolic (Intermediate JA) acid

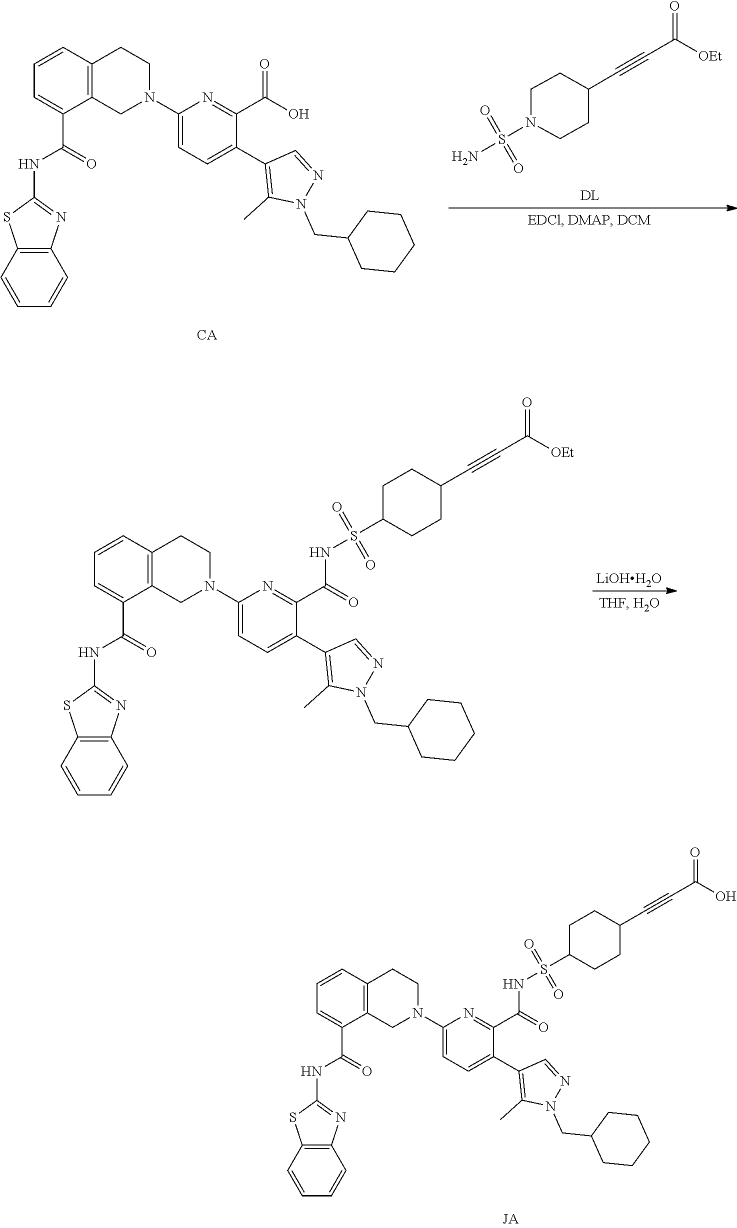 [SMILES: Cc1c(-c2ccc(N3CCc4cccc(C(=O)Nc5nc6ccccc6s5)c4C3)nc2C(=O)O)cnn1CC1CCCCC1 ; CCOC(=O)C#CC1CCN(S(N)(=O)=O)CC1 ; CCOC(=O)C#CC1CCC(S(=O)(=O)NC(=O)c2nc(N3CCc4cccc(C(=O)Nc5nc6ccccc6s5)c4C3)ccc2-c2cnn(CC3CCCCC3)c2C)CC1 ; "LiOH·H2O" ; Cc1c(-c2ccc(N3CCc4cccc(C(=O)Nc5nc6ccccc6s5)c4C3)nc2C(=O)NS(=O)(=O)C2CCC(C#CC(=O)O)CC2)cnn1CC1CCCCC1]

Step 1—Ethyl 3-(1-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)piperidin-4-yl) propiolate. To a solution of ethyl 3-(1-sulfamoyl-4-piperidyl) prop-2-ynoate (194.6 mg, 643 umol, Intermediate IZ), 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid (300 mg, 494 umol, Intermediate CA), EDCI (284 mg, 1.48 mmol), and DMAP (211 mg, 1.73 mmol) in DCM (3 mL) was degassed and purged with N₂ three times, and then the mixture was stirred at 25° C., for 3 h under N₂ atmosphere. On completion, the reaction mixture was quenched with H₂O (8 mL), and then extracted with DCM (10 mL×3). The combined organic layers were washed with brine (2 mL×3), dried over Na₂SO₄, filtered and concentrated under reduced pressure to give a residue. The residue was purified by reversed-phase HPLC (0.1% FA condition) to give the title compound (185 mg, 44% yield) as a yellow solid. LC-MS (ESI⁺) m/z 849.4 (M+H)⁺.

Step 2—3-(1-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)piperidin-4-yl) propiolic acid. To a solution of ethyl 3-(1-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)piperidin-4-yl) propiolate (165 mg, 194 umol) and LiOH·H₂O (40.8 mg, 972 umol) in THF (0.8 mL) and H₂O (0.8 mL) was degassed and purged with N₂ three times. Then the mixture was stirred at 25° C., for 2 h under N₂ atmosphere. On completion, the reaction mixture was quenched with H₂O (2 mL), and then extracted with DCM (3 mL×3). The combined aqueous phase was treated with HCl (2M) to adjust the pH=3˜4, then the mixture was extracted by DCM (2 mL×3). The combined organic phase was concentrated under reduced pressure to give the title compound (175 mg) as a white solid. LC-MS (ESI⁺) m/z 821.5 (M+H)⁺.

### 3-((tert-butoxycarbonyl)(2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)amino)propanoic acid (Intermediate JB)

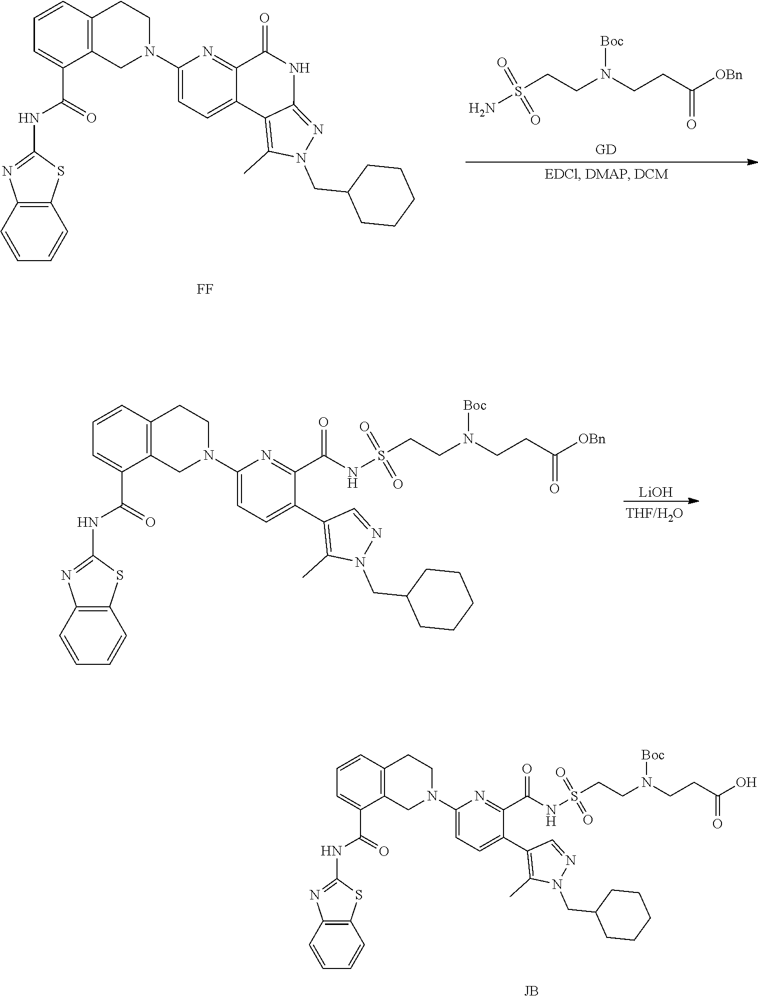 [SMILES: Cc1c2c(nn1CC1CCCCC1)[nH]c(=O)c1nc(N3CCc4cccc(C(=O)Nc5nc6ccccc6s5)c4C3)ccc12 ; CC(C)(C)OC(=O)N(CCC(=O)OCc1ccccc1)CCS(N)(=O)=O ; "FF" ; Cc1c(-c2ccc(N3CCc4cccc(C(=O)Nc5nc6ccccc6s5)c4C3)nc2C(=O)NS(=O)(=O)CCN(CCC(=O)OCc2ccccc2)C(=O)OC(C)(C)C)cnn1CC1CCCCC1 ; "LiOH" ; Cc1c(-c2ccc(N3CCc4cccc(C(=O)Nc5nc6ccccc6s5)c4C3)nc2C(=O)NS(=O)(=O)CCN(CCC(=O)O)C(=O)OC(C)(C)C)cnn1CC1CCCCC1]

Step 1—Benzyl 3-((tert-butoxycarbonyl)(2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)amino)propanoate. To a solution of benzyl 3-((tert-butoxycarbonyl)(2-sulfamoylethyl)amino)propanoate (267 mg, 691.10 umol, Intermediate GD) and 3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinic acid (300 mg, 494 umol, Intermediate FF) in DCM (3 mL) was added EDCI (189 mg, 987 umol), DMAP (151 mg, 1.23 mmol), and 4 Å molecular sieves (100 mg, 494 umol). The mixture was then stirred at 25° C., for 2 h. On completion, the reaction mixture was poured into ice water (5 mL) and extracted with DCM (5 mL×3). The combined organic layers were washed with brine (5 mL×3), dried over sodium sulfate, filtered and concentrated under reduced pressure to give a residue. The residue was purified by flash silica gel chromatography (ISCOR; 12 g SepaFlash® Silica Flash Column, Eluent of 0˜100% Ethyl acetate/Petroleum ether gradient @ 70 mL/min) to afford the title compound (280 mg, 58% yield) as a yellow solid. LC-MS (ESI⁺) m/z 976.5 (M+H)⁺.

Step 2—3-((Tert-butoxycarbonyl)(2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)amino)propanoic acid. To a solution of benzyl 3-((tert-butoxycarbonyl)(2-(N-(3-(1-(cyclohexylmethyl)-5-methyl-1H-pyrazol-4-yl)-6-(8-(thiazolo[5,4-b]pyridin-2-ylcarbamoyl)-3,4-dihydroisoquinolin-2 (1H)-yl)picolinoyl)sulfamoyl)ethyl)amino)propanoate (320 mg, 328 umol) in THF (3 mL) and H₂O (0.8 mL) was added LiOH·H₂O (68.8 mg, 1.64 mmol). The mixture was stirred at 25° C., for 12 h. On completion, the reaction mixture was poured into water (4 mL) and extracted with DCM (4 mL×3). The aqueous phase was acidified with IM HCl to pH=4, then extracted with DCM (4 mL×3). The combined organic layers were washed with brine (5 mL×3), dried over Na₂SO₄, filtered and concentrated under reduced pressure to give a residue (240 mg, 83% yield) as a yellow solid. LC-MS (ESI⁺) m z 886.4 (M+H)⁺.

### Tert-butyl 1-(3-sulfamoylpropyl)piperidine-4-carboxylate (Intermediate JC)

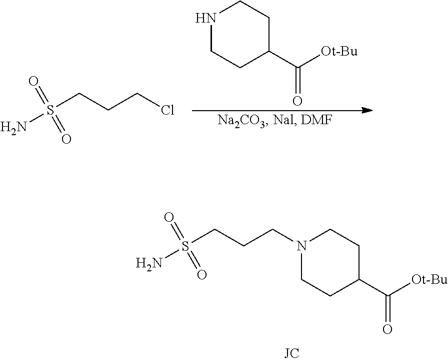

To a solution of 3-chloropropane-1-sulfonamide (2.5 g, 15.8 mmol) and tert-butyl piperidine-4-carboxylate hydrochloride (3.20 g, 14.4 mmol) in DMF (50 mL) was added Na₂CO; (9.17 g, 86.51 mmol) and Nal (2.16 g, 14.4 mmol) at 20° C. Then the reaction was stirred at 70° C., for 10 h. On completion, the reaction was extracted with ethyl acetate (2×70 mL). The combined organic phase was washed with brine (2×50 mL), dried over sodium sulfate, filtered and concentrated to give a residue. The residue was purified by column chromatography on silica gel (petroleum ether:ethyl acetate=5:1 to 3:1) to give the title compound (3.4 g, 77% yield) as a yellow solid. LC-MS (ESI⁺) m/z 307.0 (M+H)⁺.

### 1-[4-[[6-[8-(1,3-Benzothiazol-2-ylcarbamoyl)-3,4-dihydro-1H-isoquinolin-2-yl]-3-[1-(cyclohexylmethyl)-5-methylpyrazol-4-yl]pyridine-2-carbonyl]sulfamoyl]phenyl]piperidine-4-carboxylic acid (Intermediate JD)

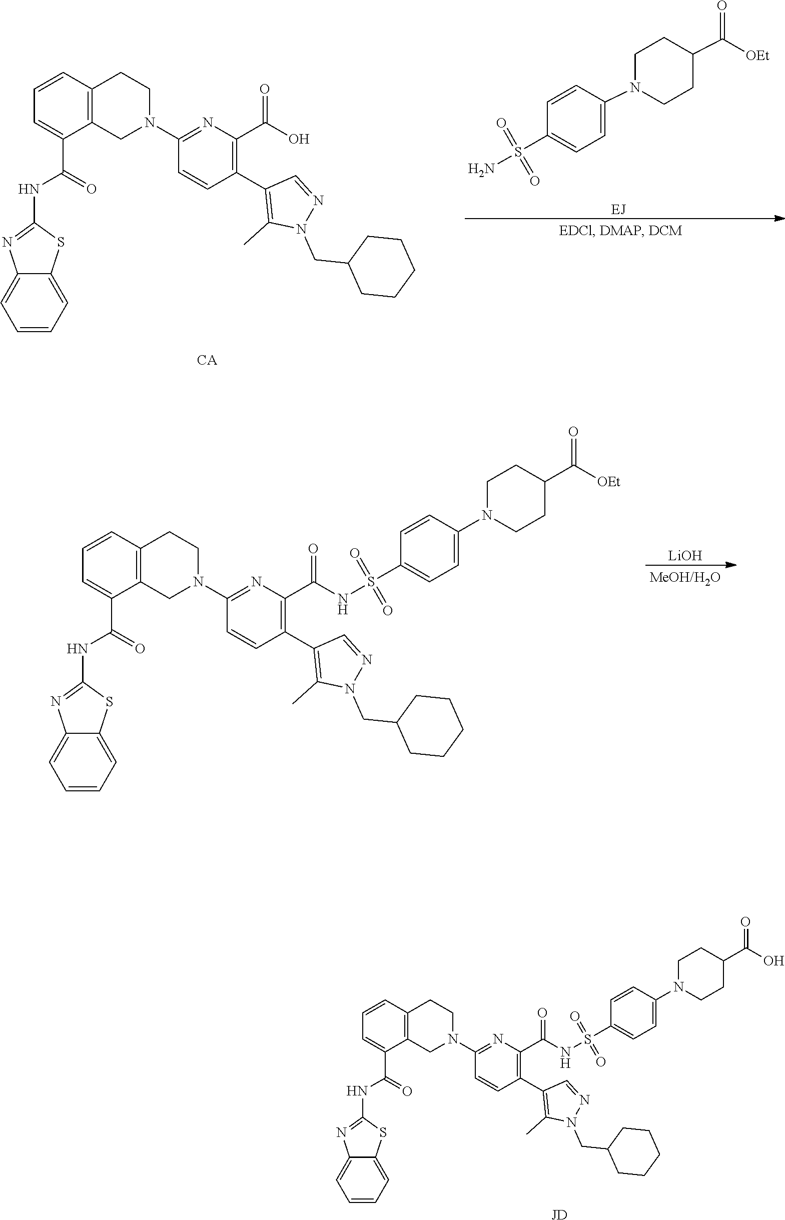

Step 1—Ethyl 1-[4-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]phenyl]piperidine-4-carboxylate. To a solution of ethyl 1-(4-sulfamoylphenyl)piperidine-4-carboxylate (154 mg, 494 umol, Intermediate EJ) and 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (200 mg, 329 umol, Intermediate CA) in DMF (2 mL) and DCM (2 mL) was added DMAP (201 mg, 1.65 mmol) and EDCI (252 mg, 1.32 mmol). The mixture was stirred at 25° C., for 2 h. On completion, the reaction was diluted with water (50 mL) and extracted with EtOAc (30 mL×2). The combined organic layer was dried over Na₂SO₄, filtered and concentrated in vacuo to give a residue. The residue was purified by column chromatography (SiO₂, PE:EtOAc=3:1 to 0:1) to give the title compound (160 mg, 53% yield) as a yellow solid. ¹H NMR (400 MHz, DMSO-d₆) δ 8.03 (d, J=8.0 Hz, 1H), 7.79 (d, J=8.0 Hz, 1H), 7.63 (d, J=7.6 Hz, 1H), 7.55 (d, J=8.4 Hz, 2H), 7.49-7.44 (m, 1H), 7.40-7.34 (m, 2H), 7.18 (s, 1H), 6.85 (d, J=7.6 Hz, 3H), 6.64 (d, J=4.8 Hz, 3H), 4.91 (s, 2H), 4.08 (q, J=7.2 Hz, 2H), 3.91 (t, J=5.6 Hz, 2H), 3.74 (d, J=7.2 Hz, 4H), 2.92 (s, 4H), 2.89-2.80 (m, 2H), 1.91-1.85 (m, 5H), 1.79-1.71 (m, 1H), 1.68-1.57 (m, 5H), 1.52 (d, J=12.0 Hz, 2H), 1.19 (t, J=7.2 Hz, 3H), 1.15 (d, J=7.2 Hz, 2H), 0.99-0.88 (m, 2H).

Step 2—1-[4-[6-[8-(1,3-Benzothiazol-2-ylcarbamoyl)-3,4-dihydro-1H-isoquinolin-2-yl]-3-[1-(cyclohexylmethyl)-5-methylpyrazol-4-yl]pyridine-2-carbonyl]sulfamoyl]phenyl]piperidine-4-carboxylic acid. To a solution of ethyl 1-[4-[[6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carbonyl]sulfamoyl]phenyl]piperidine-4-carboxylate (110 mg, 122 umol) in MeOH (2 mL) and H₂O (0.5 mL) was added LiOH·H₂O (15.3 mg, 366 umol). The mixture was stirred at 25° C., for 4 hrs. On completion, the reaction was acidified with HCl (4M) to pH=4-5 and diluted with water (20 mL) and extracted with EtOAc (2×20 mL). The combined organic layers were dried over Na₂SO₄, filtered and concentrated in vacuo to give the title compound (100 mg, 93% yield) as a yellow solid. LC-MS (ESI⁺) m/z 873.3 (M+H)⁺.

### (9H-fluoren-9-yl)methyl ((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamate (Intermediate JE)

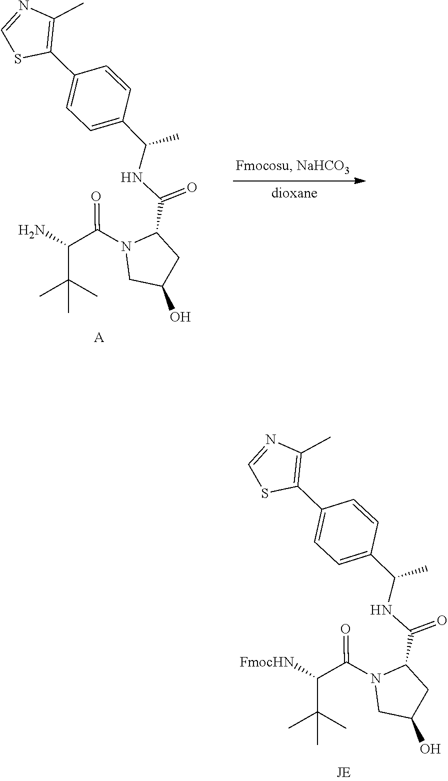

To a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (2.00 g, 4.16 mmol, HCl salt, Intermediate A) and (2,5-dioxopyrrolidin-1-yl) 9H-fluoren-9-ylmethyl carbonate (1.68 g, 4.99 mmol, CAS #82911-69-1) in dioxane (20 mL) was added NaHCO; (1.05 g, 12.5 mmol, 485 uL). The mixture was stirred at 25° C., for 4 h. On completion, the reaction mixture was quenched with NH₄Cl (20 mL), and then extracted with EtOAc (30 mL×3). The combined organic layers were washed with brine (15 mL×3), dried over sodium sulfate, filtered and concentrated under reduced pressure to give a residue. The residue was purified by column chromatography (SiO₂, Petroleum ether/Ethyl acetate=5/1 to 0/1) to give the title compound (2.00 g, 72% yield) as a yellow solid. LC-MS (ESI⁺) m/z 667.4 (M+H)⁺: ¹H NMR (400 MHZ, DMSO-d₆) δ=8.98 (s, 1H), 8.41 (br d, J=8.0 Hz, 1H), 7.89 (d, J=7.6 Hz, 2H), 7.78 (br dd, J=4.4, 7.2 Hz, 2H), 7.44-7.37 (m, 7H), 7.32 (br d, J=7.2 Hz, 2H), 5.11 (br d, J=3.6 Hz, 1H), 4.96-4.87 (m, 1H), 4.46 (br t, J=8.0 Hz, 1H), 4.26-4.18 (m, 3H), 3.68-3.54 (m, 2H), 2.59 (s, 1H), 2.46 (s, 3H), 2.07-1.97 (m, 2H), 1.44-1.33 (m, 4H), 1.01-0.95 (m, 9H).

### (3R,5S)-1-((S)-2-amino-3,3-dimethylbutanoyl)-5-(((S)-1-(4-(4-methylthiazol-5-yl)phenyl)ethyl)carbamoyl)pyrrolidin-3-yl 3-(di-tert-butoxyphosphoryl)propanoate (Intermediate JF)

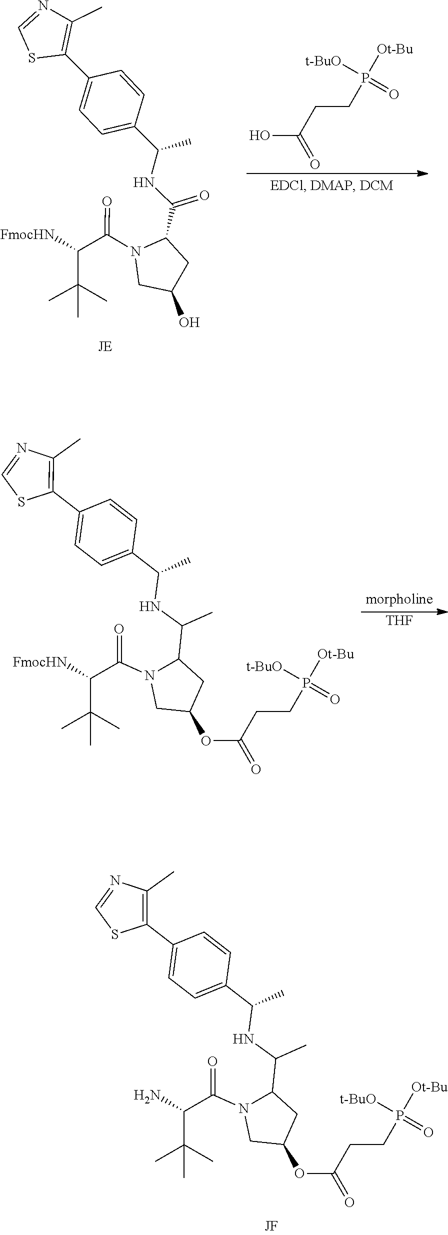

Step 1 (3R,5S)-1-((S)-2-((((9H-fluoren-9-yl)methoxy) carbonyl)amino)-3,3-dimethylbutanoyl)-5-(((S)-1-(4-(4-methylthiazol-5-yl)phenyl)ethyl)carbamoyl)pyrrolidin-3-yl3-(di-tert-butoxyphosphoryl) propanoate. To a solution of (9H-fluoren-9-yl)methyl ((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) carbamate (1.00 g, 1.50 mmol, Intermediate JE), 3-(di-tert-butoxyphosphoryl) propanoic acid (1.20 g, 4.50 mmol) in DCM (10 mL) was added EDCI (575 mg, 3.00 mmol) and DMAP (91.6 mg, 750 umol). The mixture was stirred at 40° C., for 4 h. On completion, the reaction was poured into water (10 mL) and extracted with DCM (15 mL×3). The combined organic phase is washed with brine (10 mL×2), dried over sodium sulfate, filtered and concentrated to give a residue. The residue was purified by column chromatography (SiO₂, Petroleum ether/Ethyl acetate=5/1 to 0:1) to give a title compound (1.10 g, 80% yield) as a yellow solid. LC-MS (ESI⁺) m/z 915.7 (M+H)⁺: ¹H NMR (400 MHZ, DMSO-d₆) δ=8.98 (s, 1H), 8.43 (br d, J=7.6 Hz, 1H), 7.89 (d, J=7.2 Hz, 2H), 7.78 (br dd, J=7.6, 10.8 Hz, 2H), 7.49-7.30 (m, 10H), 5.75 (s, 1H), 5.19 (br s, 1H), 4.92 (quin, J=7.2 Hz, 1H), 4.51 (br t, J=8.4 Hz, 1H), 4.32 (br dd, J=7.6, 10.0 Hz, 1H), 4.21 (br t, J=7.2 Hz, 1H), 4.13-4.06 (m, 2H), 4.00-3.92 (m, 1H), 3.73 (br dd, J=3.2, 11.2 Hz, 1H), 2.45 (s, 3H), 2.37 (td, J=7.6, 12.4 Hz, 3H), 2.30-2.22 (m, 1H), 1.88-1.71 (m, 3H), 1.30 (d, J=3.2 Hz, 18H), 1.04-0.96 (m, 9H).

Step 2—(3R,5S)-1-((S)-2-amino-3,3-dimethylbutanoyl)-5-(((S)-1-(4-(4-methylthiazol-5-yl)phenyl)ethyl)carbamoyl)pyrrolidin-3-yl 3-(di-tert-butoxyphosphoryl) propanoate. To a solution of (3R,5S)-1-((S)-2-((((9H-fluoren-9-yl)methoxy) carbonyl)amino)-3,3-dimethylbutanoyl)-5-(((S)-1-(4-(4-methylthiazol-5-yl)phenyl)ethyl)carbamoyl)pyrrolidin-3-yl3-(di-tert-butoxyphosphoryl) propanoate (1.10 g, 1.20 mmol) in THF (10 mL) was added morpholine (419 mg, 4.81 mmol, 423 uL). The mixture was stirred at 0-25° C., for 2 h. On completion, the reaction was poured into water (10 mL) and extracted with EtOAc (20 mL×3). The combined organic phase is washed with brine (10 mL×2), dried over sodium sulfate, filtered and concentrated to give a residue. The residue was purified by column chromatography prep-TLC (SiO₂, Petroleum ether/Ethyl acetate=3:1 to Dichloromethane:Methanol=20:1) to give the title compound (450 mg, 54% yield) as a yellow oil. LC-MS (ESI⁺) m/z 693.2 (M+H)⁺.

### (R)-methyl 1-(4-sulfamoylphenyl) pyrrolidine-3-carboxylate (Intermediate JG)

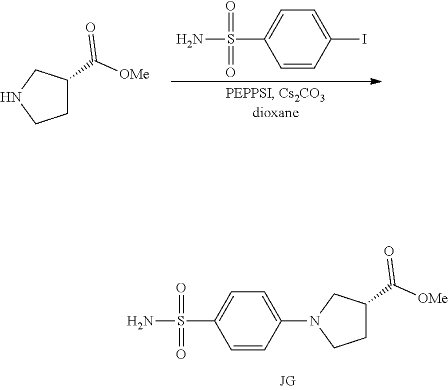 [SMILES: COC(=O)[C@@H]1CCNC1 ; NS(=O)(=O)c1ccc(I)cc1 ; COC(=O)[C@@H]1CCN(c2ccc(S(N)(=O)=O)cc2)C1]

To a solution of 4-iodobenzenesulfonamide (500 mg, 1.77 mmol, CAS #825-86-5) and (R)-methyl pyrrolidine-3-carboxylate (878 mg, 5.30 mmol, HCl, CAS #428518-43-8) in dioxane (20 mL) was added Pd-PEPPSI-IHeptCl (172 mg, 177 umol) and Cs₂CO; (2.30 g, 7.06 mmol). The mixture was stirred at 100° C., for 2 h under N₂ atmosphere. On completion, the reaction mixture was quenched with water (20 mL) at 20° C., and extracted with EtOAc (40 mL×2). The combined organic layers were washed with brine (80 mL×2), dried over Na₂SO₄, filtered and concentrated under reduced pressure to give a residue. The crude product was purified by reversed-phase HPLC (0.1% FA condition) to give the title compound (356 mg, 71% yield) as a white solid. LC-MS (ESI⁺) m/z 285.0 (M+H)⁺.

### (R)-1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylic acid (Intermediate JH)

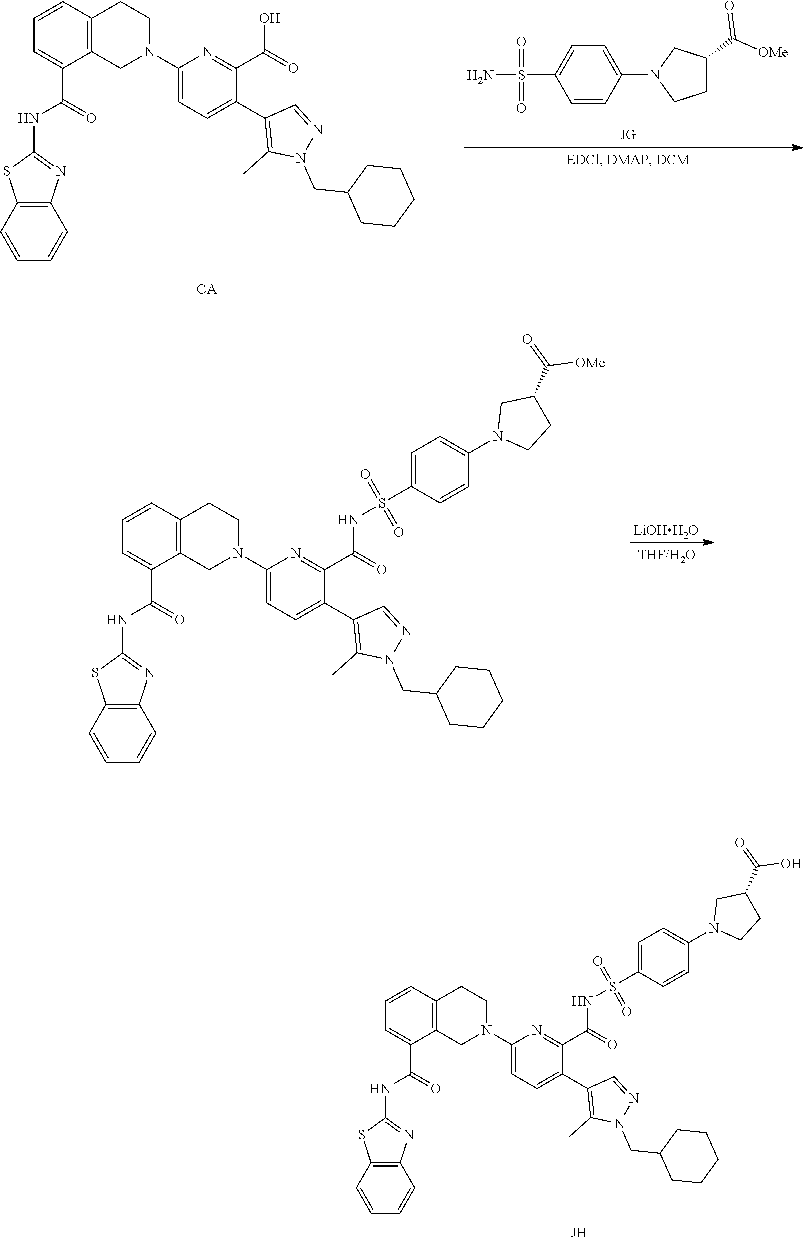 [SMILES: Cc1c(-c2ccc(N3CCc4cccc(C(=O)Nc5nc6ccccc6s5)c4C3)nc2C(=O)O)cnn1CC1CCCCC1 ; COC(=O)[C@@H]1CCN(c2ccc(S(N)(=O)=O)cc2)C1 ; COC(=O)[C@@H]1CCN(c2ccc(S(=O)(=O)NC(=O)c3nc(N4CCc5cccc(C(=O)Nc6nc7ccccc7s6)c5C4)ccc3-c3cnn(CC4CCCCC4)c3C)cc2)C1 ; "LiOH·H2O" ; Cc1c(-c2ccc(N3CCc4cccc(C(=O)Nc5nc6ccccc6s5)c4C3)nc2C(=O)NS(=O)(=O)c2ccc(N3CC[C@@H](C(=O)O)C3)cc2)cnn1CC1CCCCC1]

Step 1—(R)-methyl 1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylate. To a solution of methyl (R)-methyl 1-(4-sulfamoylphenyl) pyrrolidine-3-carboxylate (356 mg, 1.25 mmol, Intermediate JG), 6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinic acid (506 mg, 835 umol, Intermediate CA) and 4 Å molecular sieves (534 mg) in DCM (4 mL) was added DMAP (408 mg, 3.34 mmol) and EDCI (320 mg, 1.67 mmol). The mixture was stirred at 25° C., for 12 h. On completion, the reaction mixture was quenched with water (4 mL) at 20° C., and extracted with DCM (4 mL×2). The combined organic layers were washed with brine (10 mL×2), dried over Na₂SO₄, filtered and concentrated under reduced pressure to give a residue. The residue was purified by column chromatography (SiO₂, Petroleum ether/Ethyl acetate=3/1 to 1/2) and purified by reversed-phase HPLC (0.1% FA condition) to give the title compound (320 mg, 44% yield) as a yellow solid. LC-MS (ESI⁺) m/z 873.3 (M+H)⁺.

Step 2—(R)-1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylic acid. To a solution of methyl (R)-methyl 1-(4-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl) pyrrolidine-3-carboxylate (290 mg, 332 umol) in THF (2.25 mL) and H₂O (0.75 mL) was added LiOH·H₂O (69.7 mg, 1.66 mmol). The mixture was stirred at 25° C., for 12 h. On completion, the reaction mixture was quenched with water (3 mL) at 20° C., then the aqueous was adjust to pH=3˜4 and extracted with DCM (3 mL×2). The organic layers were washed with brine (6 mL×2), dried over Na₂SO₄, filtered and concentrated under reduced pressure to give the title compound (220 mg, 77% yield) as a yellow solid. LC-MS (ESI⁺) m/z 859.4 (M+H)⁺.

### Ethyl 1-(3-sulfamoylphenyl)piperidine-4-carboxylate (Intermediate JI)

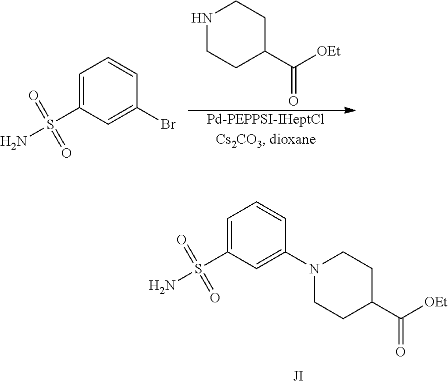

To a solution of ethyl piperidine-4-carboxylate (1.33 g, 8.47 mmol, 1.31 mL, CAS #1126 Sep. 6) and 3-bromobenzenesulfonamide (500 mg, 2.12 mmol, CAS #1126 Sep. 6) in dioxane (20 mL) was added Cs₂CO₃ (2.76 g, 8.47 mmol) and 1,3-bis[2,6-bis(1-propylbutyl)phenyl]-4,5-dichloro-2H-imidazol-1-ium-2-ide: 3-chloropyridine: dichloropalladium (206.0 mg, 211.8 umol). The mixture was stirred at 100° C., for 4 hr. On completion, the reaction mixture was quenched with H₂O (5 mL) at 25° C., and then diluted with DCM (10 mL) and extracted with DCM (10 mL×3). The combined organic layers were washed with H₂O (5 mL×3), dried over Na₂SO₄ filtered and concentrated under reduced pressure to give a residue. The residue was purified by column chromatography (SiO₂, Petroleum ether/Ethyl acetate=5/1 to 1/1) to afford the title compound (300.0 mg, 45% yield) as a yellow solid. ¹H NMR (400 MHZ, DMSO-d₆) δ=12.86 (br s, 1H), 12.12 (br d, J=2.4 Hz, 1H), 8.02 (d, J=8.0 Hz, 1H), 7.79 (br d, J=8.0 Hz, 1H), 7.72 (br s, 1H), 7.63 (br d, J=6.4 Hz, 1H), 7.50-7.42 (m, 5H), 7.40-7.32 (m, 3H), 7.06 (s, 1H), 6.98 (br d, J=8.0 Hz, 1H), 5.75 (s, 1H), 4.95 (br s, 2H), 4.02 (q, J=7.2 Hz, 3H), 3.92 (br t, J=6.0 Hz, 2H), 3.74 (d, J=7.2 Hz, 2H), 3.02 (br t, J=5.2 Hz, 2H), 2.89 (s, 1H), 2.73 (s, 1H), 2.66 (br t, J=7.6 Hz, 3H), 2.29 (t, J=7.2 Hz, 2H), 1.86-1.78 (m, 5H), 1.49 (br d, J=13.2 Hz, 2H), 1.23 (s, 1H), 1.18-1.12 (m, 6H), 0.91 (br d, J=11.6 Hz, 2H).

### 1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)piperidine-4-carboxylic acid (Intermediate JJ)

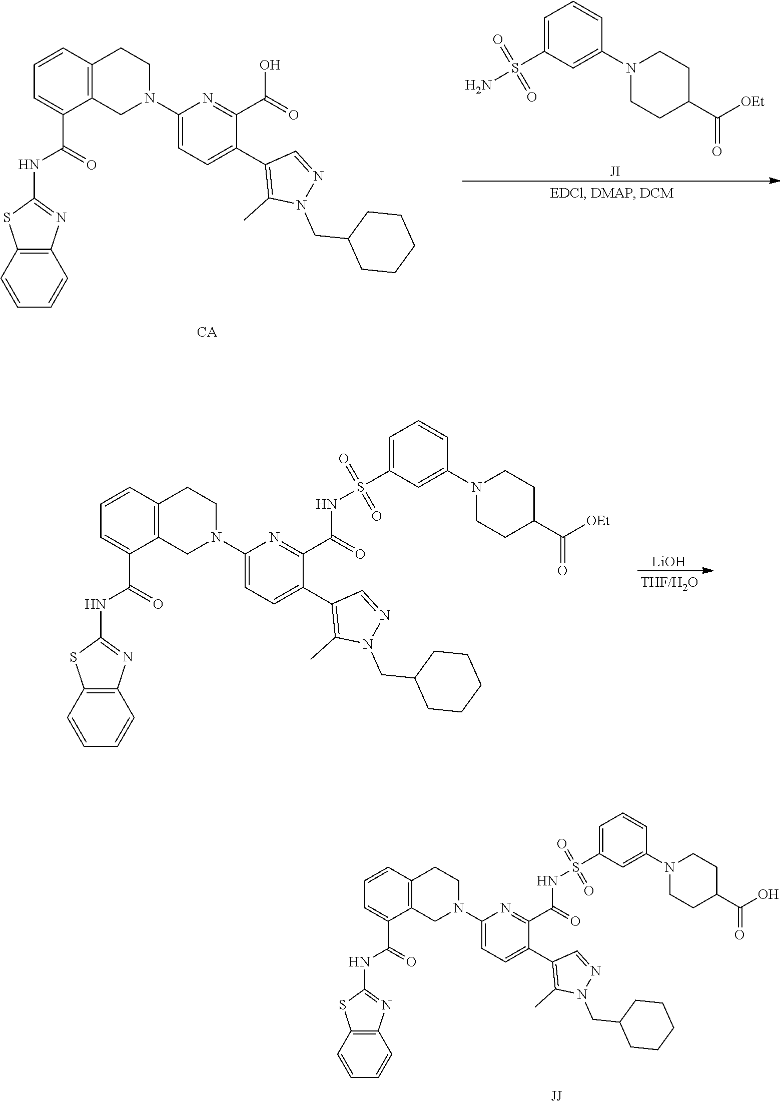 [SMILES: Cc1c(-c2ccc(N3CCc4cccc(C(=O)Nc5nc6ccccc6s5)c4C3)nc2C(=O)O)cnn1CC1CCCCC1 ; CCOC(=O)C1CCN(c2cccc(S(N)(=O)=O)c2)CC1 ; "JI" ; CCOC(=O)C1CCN(c2cccc(S(=O)(=O)NC(=O)c3nc(N4CCc5cccc(C(=O)Nc6nc7ccccc7s6)c5C4)ccc3-c3cnn(CC4CCCCC4)c3C)c2)CC1 ; "LiOH" ; Cc1c(-c2ccc(N3CCc4cccc(C(=O)Nc5nc6ccccc6s5)c4C3)nc2C(=O)NS(=O)(=O)c2cccc(N3CCC(C(=O)O)CC3)c2)cnn1CC1CCCCC1]

Step 1—Ethyl 1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)piperidine-4-carboxylate. To a solution of 6-[8-(1,3-benzothiazol-2-ylcarbamoyl)-3,4-dihydro-1H-isoquinolin-2-yl]-3-[1-(cyclohexylmethyl)-5-methyl-pyrazol-4-yl]pyridine-2-carboxylic acid (353 mg, 582 umol, Intermediate CA) and ethyl 1-(3-sulfamoylphenyl)piperidine-4-carboxylate (200 mg, 640 umol, Intermediate JI) in DCM (3 mL) was added EDCI (223 mg, 1.16 mmol) and DMAP (178 mg, 1.46 mmol). The mixture was stirred at 40° C., for 4 hr. On completion, the reaction mixture was quenched with H₂O (5 mL) at 25° C., and then diluted with DCM (10 mL) and extracted with DCM (10 mL×3). The combined organic layers were washed with water (5 mL×3), dried over Na₂SO₄ filtered and concentrated under reduced pressure to give a residue. The crude product was purified by reversed-phase HPLC (ACN/0.1% FA=65%) to give the title compound (300 mg, 57% yield) as a yellow solid. LC-MS (ESI⁺) m/z 901.4 (M+H)⁺.

Step 2—1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)piperidine-4-carboxylic acid. To a solution of ethyl 1-(3-(N-(6-(8-(benzo[d]thiazol-2-ylcarbamoyl)-3,4-dihydroisoquinolin-2 (1H)-yl)-3-(1-(cyclohexylmethyl)-5-methyl-1H-pyrazol-4-yl)picolinoyl)sulfamoyl)phenyl)piperidine-4-carboxylate (300 mg, 333 umol) in THF (2 mL) and H₂O (2 mL) was added LiOH·H₂O (69.9 mg, 1.66 mmol). The mixture was then stirred at 25° C., for 2 h. On completion, the reaction mixture was diluted with water (2 mL) and the combined organic layers were extracted with DCM (3 mL). The pH of the mixture was then adjusted to 5˜6 with 2 M HCl. The mixture was filtered and concentrated under reduced pressure to give the title compound (290 mg, 97% yield) as a yellow solid. LC-MS (ESI⁺) m/z 873.3 (M+H)⁺.

### (2S,4R)-1-((S)-2-amino-3,3-dimethylbutanoyl)-4-hydroxy-N-((R)-2-hydroxy-1-(4-(4-methylthiazol-5-yl)phenyl)ethyl)pyrrolidine-2-carboxamide (Intermediate JK)

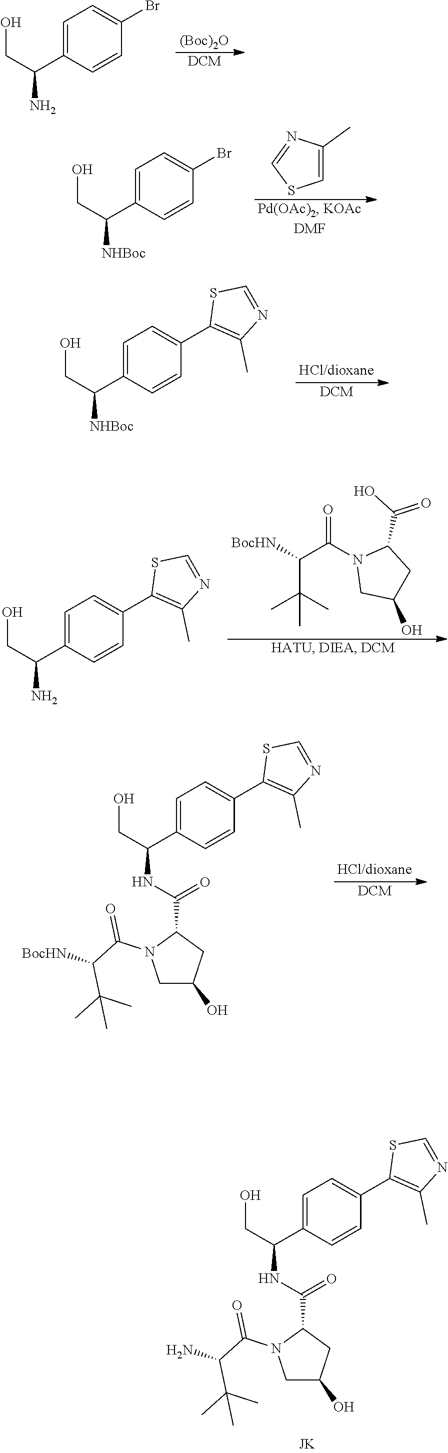

Step 1—(R)-tert-butyl (1-(4-bromophenyl)-2-hydroxyethyl)carbamate. To a solution of (2R)-2-amino-2-(4-bromophenyl)ethanol (1.00 g, 4.63 mmol, CAS #354153-64-3) in DCM (20 mL) was added (Boc) 20 (1.11 g, 5.09 mmol, 1.17 mL). The mixture was stirred at 20° C., for 2 hrs. On completion, the reaction mixture was concentrated under reduced pressure to give the title compound (1.41 g) as a white solid. LC-MS (ESI) m/z 259.8 (M-55); ¹H NMR (400 MHz. DMSO-d₆) δ=7.49 (d, J=8.4 Hz, 2H), 7.24 (d, J=8.4 Hz, 3H), 4.80 (t, J=5.6 Hz, 1H), 4.48 (d, J=6.8 Hz. IH), 3.50-3.42 (m, 2H), 1.36 (s, 9H).

Step 2—(R)-tert-butyl (2-hydroxy-1-(4-(4-methylthiazol-5-yl)phenyl)ethyl)carbamate. A mixture of tert-butyl N-](IR)-1-(4-bromophenyl)-2-hydroxy-ethyl]carbamate (1.4 g, 4.43 mmol). 4-methylthiazole (1.10 g, 11.1 mmol. 1.01 mL. CAS #693-95-8). KOAc (869 mg. 8.86 mmol), and Pd (OAc) 2 (99.4 mg. 443 umol) in DMF (30 mL) was degassed and purged with N₂ three times, and then the mixture was stirred at 100° C., for 12 hrs under N₂ atmosphere. The reaction mixture was filtered to give a liquid, and then diluted with H₂O (30 mL) and extracted with EA (40 mL×3). The combined organic layers were washed with brine (40 mL×3), dried over Na₂SO₄, filtered and concentrated under reduced pressure to give a residue. The residue was purified by column chromatography (SiO₂. Petroleum ether/Ethyl acetate=I/O to 1/1) to give the title compound (800 mg. 49% yield) as a yellow solid. LC-MS (ESI) m/z 335.2 (M+H).

Step 3—(R)-2-amino-2-(4-(4-methylthiazol-5-yl)phenyl)ethanol. To a solution of tert-butyl N—[(IR)-2-hydroxy-1-[4-(4-methylthiazol-5-yl)phenyl]ethyl]carbamate (800 mg. 2.39 mmol) in DCM (12 mL) was added HCl/dioxane (4 M. 3 mL). Then the mixture was stirred at 20° C., for 2 hrs. On completion, the reaction mixture was concentrated under reduced pressure to give the title compound (670 mg. HCl) as a yellow solid. LC-MS (ESI) m/z 235.1 (M+H).

Step 4—Tert-butyl ((S)-1-((2S,4R)-4-hydroxy-2-(((R)-2-hydroxy-1-(4-(4-methylthiazol-5-yl)phenyl)ethyl)carbamoyl)pyrrolidin-1-yl)-3,3-dimethyl-1-oxobutan-2-yl) carbamate. To a solution of (2S,4R)-1-[(2S)-2-(tert-butoxycarbonylamino)-3,3-dimethyl-butanoyl]-4-hydroxy-pyrrolidine-2-carboxylic acid (254 mg. 739 umol. CAS #630421-46-4) in DCM (8 mL) was added HATU (421 mg, 1.11 mmol) and DIEA (382 mg. 2.95 mmol) at 0° C. After addition, the mixture was stirred at this temperature for 0.5 hr, and then (2R)-2-amino-2-[4-(4-methylthiazol-5-yl)phenyl]ethanol (200 mg. 739 umol. HCl) was added at 0° C. The resulting mixture was stirred at 0° C., for 0.5 hr. On completion, the reaction mixture was diluted with H₂O (20 mL) and extracted with DCM (15 mL×3). The combined organic layers were dried over Na₂SO₄, filtered and concentrated under reduced pressure to give a residue. The residue was purified by column chromatography (SiO₂. Petroleum ether/Ethyl acetate=1/2 to 0/1) to give the title compound (348 mg. 83% yield) as a white solid. LC-MS (ESI) m/z 561.1 (M+H); ¹H NMR (400 MHZ. DMSO-d₆) ô=8.98 (s, 1H), 8.39 (d, J=7.6 Hz, 1H), 8.24-8.11 (m, 1H), 7.46-7.36 (m, 4H), 6.43 (d, J=9.2 Hz, 1H), 5.12 (d, J=3.2 Hz, 1H), 4.90-4.81 (m, 1H), 4.76 (t, J=6.0 Hz, 1H), 4.49 (t, J=8.0 Hz. IH), 4.29 (s, 1H), 4.14 (d, J=9.2 Hz, 1H), 3.65-3.55 (m. 5H), 3.14 (dt, J=3.2, 7.2 Hz, 1H), 2.46 (s, 3H), 2.07-2.00 (m. IH), 1.85-1.77 (m. IH), 1.41-1.36 (m. 9H), 0.93 (s, 9H).

Step 5—(2S,4R)-1-((S)-2-amino-3,3-dimethylbutanoyl)-4-hydroxy-N-((R)-2-hydroxy-1-(4-(4-methylthiazol-5-yl)phenyl)ethyl)pyrrolidine-2-carboxamide. To a solution of tert-butyl N-[(1S)-1-[(2S,4R)-4-hydroxy-2-[[(1R)-2-hydroxy-1-[4-(4-methylthiazol-5-yl)phenyl]ethyl]carbamoyl]pyrrolidine-1-carbonyl]-2,2-dimethyl-propyl]carbamate (340 mg, 606 umol) in DCM (12 mL) was added HCl/dioxane (4 M, 3 mL). Then the mixture was stirred at 25° C., for 1 hr. On completion, the reaction mixture was filtered to give the title compound (280 mg, HCl) as a yellow solid. LC-MS (ESI⁺) m/z 461.2 (M+H)⁺.

### Example 1 (Method 1); Synthesis of (2S,4R)-1-((S)-2-(10-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-10-oxodecanamido)-3,3-dimethylbutanoyl)-4-hydroxy-N-((S)-1-(4-(4-methylthiazol-5-yl)phenyl)ethyl)pyrrolidine-2-carboxamide (I-1)

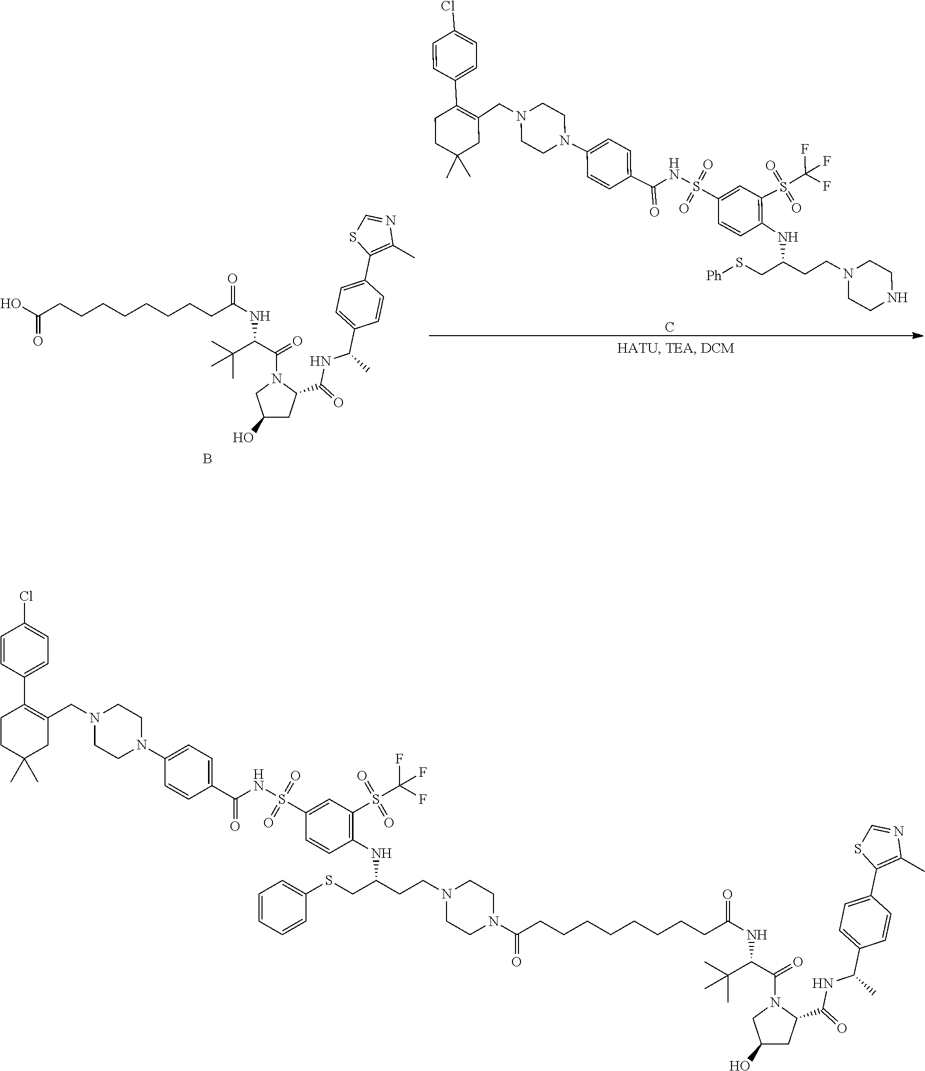

A mixture of 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (32.3 mg, 0.051 mmol, Intermediate B), (R)-4-(4-((4′-chloro-4,4-dimethyl-3,4,5,6-tetrahydro-[1, l′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (50.0 mg, 0.0510 mmol, Intermediate C), HATU (20.5 mg, 0.0539 mmol) and TEA (26.0 mg, 0.257 mmol) in DCM (1 mL) was stirred at 25° C., for 1 h under N₂ atmosphere. On completion, the reaction mixture was filtered and concentrated under reduced pressure to give a residue. The residue was purified by reversed-phase HPLC (Waters xbridge 150*25 mm 10 um; mobile phase: [water (NH₄HCO₃)-ACN]; B %: 58%-88%, 11 min) to give the title compound (30.0 mg, 37% yield) as white solid. LC-MS (ESI⁺) m/z 793.0 (1/2M+2)⁺; ¹H NMR (400 MHZ, DMSO-d₆) δ=8.99 (s, 1H), 8.37 (d, J=7.6 Hz, 1H), 8.13 (s, 1H), 7.97-7.93 (m, 1H), 7.78 (d, J=9.2 Hz, 1H), 7.72 (br d, J=9.2 Hz, 2H), 7.45-7.43 (m, 2H), 7.38 (d, J=8.4 Hz, 4H), 7.35-7.32 (m, 2H), 7.27 (t, J=7.6 Hz, 2H), 7.19 (d, J=7.6 Hz, 1H), 7.13 (br d, J=8.4 Hz, 2H), 7.03-6.96 (m, 1H), 6.88-6.84 (m, 2H), 5.09 (d, J=3.6 Hz, 1H), 4.92 (t, J=7.4 Hz, 1H), 4.52 (d, J=9.2 Hz, 1H), 4.42 (t, J=8.2 Hz, 1H), 4.30-4.27 (m, IH), 4.12-4.05 (m, 1H), 3.61 (br d, J=4.0 Hz, 2H), 3.39-3.35 (m, 4H), 3.25-3.17 (m, 4H), 2.53 (d, J=2.0 Hz, 12H), 2.46 (s, 3H), 2.27-2.20 (m, 8H), 2.14-2.08 (m, 2H), 2.00 (br s, 4H), 1.80 (ddd, J=4.8, 8.0, 12.8 Hz, 2H), 1.47-1.42 (m, 6H), 1.38 (d, J=6.8 Hz, 3H), 1.23 (br s, 8H), 0.98 (s, 6H), 0.94 (s, 9H).

TABLE 3
Compounds synthesized via Method 1, using the corresponding amines and acids for the coupling.

| | | | LCMS | |
|---|---|---|---|---|
| | | | (ESI+) | ¹H NMR |
| | | | m/z | (400 MHz, |
| I-#^{a} | Amine | Acid | (M + H)⁺ | DMSO-d6) |
| I-2^{e} | U | K | 1196.4 | 9.01-8.93 (m, 1H), 8.38 (br d, J = 8.0 Hz, 1H), 8.30 (s, 1H), 7.98 |
| | | | | (br d, J = 8.0 Hz, 1H), 7.88-7.79 (m, 1H), 7.74 (br d, J = 8.0 Hz, |
| | | | | 1H), 7.68 (br d, J = 7.2 Hz, 1H), 7.46-7.34 (m, 7H), 7.34-7.27 |
| | | | | (m, 2H), 7.26-7.13 (m, 2H), 7.07 (br d, J = 1.2 Hz, 1H), 4.93- |
| | | | | 4.88 (m, 1H), 4.84 (br s, 1H), 4.51 (br d, J = 9.2 Hz, 1H), 4.42 (br |
| | | | | t, J = 8.0 Hz, 1H), 4.35 (br s, 1H), 4.27 (br s, 1H), 4.06 (br t, J = |
| | | | | 5.6 Hz, 2H), 3.69 (br s, 3H), 3.60 (br s, 2H), 3.16 (br d, J = 7.2 Hz, |
| | | | | 3H), 3.01 (s, 3H), 2.88 (s, 1H), 2.44 (s, 2H), 2.32-2.26 (m, 2H), |
| | | | | 2.21 (dt, J = 7.2, 14.4 Hz, 2H), 2.00 (br t, J = 7.6 Hz, 3H), 1.81- |
| | | | | 1.69 (m, 3H), 1.36 (br d, J = 7.2 Hz, 2H), 1.23 (br d, J = 3.2 Hz, |
| | | | | 3H), 0.92 (br d, J = 5.2 Hz, 9H) |
| I-3^{e} | U | AC | 1224.4 | 8.98 (s, 1H), 8.35 (d, J = 7.6 Hz, 1H), 8.13 (s, 1H), 8.03 (d, J = 7.6 |
| | | | | Hz, 1H), 7.83-7.71 (m, 2H), 7.71-7.63 (m, 1H), 7.50-7.41 (m, |
| | | | | 4H), 7.39-7.35 (m, 3H), 7.34-7.25 (m, 2H), 7.23-7.16 (m, 1H), |
| | | | | 7.13-7.02 (m, 1H), 5.08 (d, J = 3.2 Hz, 1H), 4.95-4.89 (m, 1H), |
| | | | | 4.82 (s, 2H), 4.51 (d, J = 9.2 Hz, 1H), 4.44-4.33 (m, 3H), 4.27 (br |
| | | | | d, J = 2.4 Hz, 1H), 4.07 (br t, J = 6.0 Hz, 2H), 3.71 (br t, J = 5.6 |
| | | | | Hz, 2H), 3.60 (br s, 2H), 3.16 (br t, J = 6.8 Hz, 2H), 3.06-2.99 (m, |
| | | | | 4H), 2.88 (s, 1H), 2.45 (br s, 3H), 2.05-1.98 (m, 3H), 1.83-1.74 |
| | | | | (m, 1H), 1.54-1.45 (m, 4H), 1.37 (br d, J = 7.2 Hz, 3H), 1.28- |
| | | | | 1.23 (m, 4H), 0.93 (br s, 9H) |
| I-6^{e} | U | W | 629.2^{d} | 12.91-12.86 (m, 1H), 8.98-8.95 (m, 1H), 8.49-8.44 (m, 1H), |
| | | | | 8.03 (d, J = 8.4 Hz, 1H), 7.82-7.76 (m, 1H), 7.68-7.63 (m, 1H), |
| | | | | 7.49-7.33 (m, 6H), 7.29-7.23 (m, 2H), 7.19-7.15 (m, 1H), 7.11- |
| | | | | 7.06 (m, 1H), 7.01-6.98 (m, 1H), 6.96-6.92 (m, 1H), 5.19-5.10 |
| | | | | (m, 1H), 4.82 (s, 2H), 4.58 (br d, J = 9.2 Hz, 1H), 4.54-4.48 (m, |
| | | | | 1H), 4.42 (s, 1H), 4.38 (s, 1H), 4.35-4.32 (m, 1H), 4.30-4.23 (m, |
| | | | | 2H), 4.09-4.03 (m, 4H), 3.74-3.70 (m, 2H), 3.64-3.57 (m, 2H), |
| | | | | 3.20-3.13 (m, 3H), 3.06 (s, 2H), 3.04-3.01 (m, 2H), 2.91 (s, 1H), |
| | | | | 2.45-2.44 (m, 3H), 2.04-1.99 (m, 4H), 1.91 (br dd, J = 5.2, 8.8 |
| | | | | Hz, 1H), 1.39-1.32 (m, 2H), 1.24-1.19 (m, 3H), 1.16-1.13 (m, |
| | | | | 1H), 0.95 (s, 9H) |
| I-7^{e} | U | E | 643.2^{d} | 9.00-8.91 (m, 1H), 8.48 (s, 1H), 8.20 (s, 1H), 8.02 (br d, J = 7.6 |
| | | | | Hz, 1H), 7.78 (br d, J = 8.0 Hz, 1H), 7.66 (br d, J = 7.2 Hz, 1H), |
| | | | | 7.44 (br d, J = 7.6 Hz, 2H), 7.41-7.32 (m, 4H), 7.30-7.24 (m, |
| | | | | 2H), 7.19-7.16 (m, 1H), 7.11-7.05 (m, 1H), 6.99-6.92 (m, 2H), |
| | | | | 4.82 (br s, 2H), 4.59 (d, J = 9.2 Hz, 1H), 4.51 (t, J = 8.0 Hz, 1H), |
| | | | | 4.39 (s, 1H), 4.35 (s, 2H), 4.27 (br d, J = 5.2 Hz, 1H), 4.21 (br s, |
| | | | | 1H), 4.09-4.00 (m, 4H), 3.70 (br d, J = 5.2 Hz, 2H), 3.04 (s, 3H), |
| | | | | 2.89 (br d, J = 1.6 Hz, 2H), 2.45 (br s, 3H), 2.07 (s, 3H), 2.00 (br |
| | | | | d, J = 9.2 Hz, 3H), 1.76 (br d, J = 6.6 Hz, 2H), 1.71 (br s, 1H), 1.61- |
| | | | | 1.56 (m, 2H), 1.50-1.46 (m, 2H), 1.39-1.32 (m, 3H), 1.23 (s, |
| | | | | 3H), 1.17 (s, 1H), 0.95 (s, 9H) |
| I-9^{e} | AD | K | 1200.1 | 13.00-12.77 (m, 1H), 9.03-8.94 (m, 1H), 8.36 (d, J = 7.6 Hz, |
| | | | | 1H), 8.06-8.01 (m, 1H), 7.82-7.75 (m, 2H), 7.67 (d, J = 7.2 Hz, |
| | | | | 1H), 7.49-7.40 (m, 5H), 7.40-7.29 (m, 4H), 7.08-6.96 (m, 3H), |
| | | | | 5.16-5.00 (m, 1H), 4.94-4.89 (m, 1H), 4.83 (s, 2H), 4.50 (dd, J = |
| | | | | 4.8, 9.2 Hz, 1H), 4.44-4.38 (m, 1H), 4.27 (s, 1H), 4.03-3.97 |
| | | | | (m, 2H), 3.73 (t, J = 5.6 Hz, 2H), 3.59 (s, 3H), 3.23-3.19 (m, 2H), |
| | | | | 3.03 (t, J = 5.6 Hz, 2H), 2.87 (s, 1H), 2.76 (s, 1H), 2.62-2.58 (m, |
| | | | | 2H), 2.45 (s, 3H), 2.28-2.08 (m, 5H), 2.04-1.96 (m, 3H), 1.83- |
| | | | | 1.61 (m, 6H), 1.36 (d, J = 6.8 Hz, 3H), 0.91 (d, J = 4.0 Hz, 9H) |
| I-10^{e} | AD | AC | 1228.4 | 8.99 (s, 1H), 8.38 (br d, J = 7.5 Hz, 1H), 8.02 (br d, J = 7.6 Hz, |
| | | | | 1H), 7.78 (br d, J = 8.5 Hz, 2H), 7.68 (br d, J = 6.8 Hz, 1H), 7.54- |
| | | | | 7.28 (m, 8H), 7.12-6.92 (m, 3H), 4.94-4.89 (m, 1H), 4.89-4.78 |
| | | | | (m, 2H), 4.50 (br d, J = 8.9 Hz, 1H), 4.42 (br t, J = 7.8 Hz, 1H), |
| | | | | 4.27 (br s, 1H), 4.05-3.94 (m, 2H), 3.78-3.69 (m, 2H), 3.59 (br |
| | | | | s, 2H), 3.27-3.18 (m, 4H), 3.04 (br s, 2H), 2.89 (s, 2H), 2.76 (s, |
| | | | | 1H), 2.46 (s, 3H), 2.24-1.93 (m, 7H), 1.87-1.59 (m, 3H), 1.57- |
| | | | | 1.31 (m, 8H), 1.27-1.14 (m, 3H), 0.96-0.86 (m, 9H) |
| I-11^{e} | AD | B | 1270.5 | 13.05-12.73 (m, 1H), 8.98 (s, 1H), 8.36 (d, J = 7.9 Hz, 1H), 8.03 |
| | | | | (d, J = 7.9 Hz, 1H), 7.77 (br dd, J = 8.8, 16.8 Hz, 2H), 7.67 (br d, |
| | | | | J = 7.0 Hz, 1H), 7.50-7.32 (m, 8H), 7.10-6.94 (m, 3H), 5.09 (br |
| | | | | s, 1H), 4.91 (br t, J = 7.4 Hz, 1H), 4.82 (s, 2H), 4.50 (d, J = 9.3 Hz, |
| | | | | 1H), 4.42 (t, J = 8.0 Hz, 1H), 4.27 (br s, 1H), 4.04-3.95 (m, 2H), |
| | | | | 3.73 (br t, J = 5.8 Hz, 2H), 3.59 (br s, 2H), 3.26-3.16 (m, 4H), |
| | | | | 3.03 (br t, J = 5.8 Hz, 2H), 2.89 (s, 2H), 2.75 (s, 1H), 2.45 (s, 3H), |
| | | | | 2.24-1.97 (m, 7H), 1.85-1.60 (m, 3H), 1.43 (br dd, J = 7.1, 16.7 |
| | | | | Hz, 4H), 1.37 (d, J = 7.0 Hz, 3H), 1.26-1.09 (m, 9H), 0.92 (s, 9H) |
| I-12^{e} | AD | W | 1260.3 | 13.11-12.63 (m, 1H), 8.95 (d, J = 2.0 Hz, 1H), 8.47 (br t, J = 5.9 |
| | | | | Hz, 1H), 8.03 (d, J = 7.9 Hz, 1H), 7.79 (d, J = 7.8 Hz, 1H), 7.67 |
| | | | | (br d, J = 7.1 Hz, 1H), 7.51-7.32 (m, 5H), 7.28 (br d, J = 6.9 Hz, |
| | | | | 1H), 7.08-6.89 (m, 5H), 5.24-5.09 (m, 1H), 4.82 (br s, 2H), 4.59 |
| | | | | (d, J = 9.3 Hz, 1H), 4.52 (t, J = 8.2 Hz, 1H), 4.39-4.15 (m, 3H), |
| | | | | 4.07-3.93 (m, 4H), 3.76-3.68 (m, 2H), 3.67-3.57 (m, 2H), 3.27- |
| | | | | 3.15 (m, 4H), 3.02 (br t, J = 5.5 Hz, 2H), 2.92 (s, 2H), 2.78 (s, |
| | | | | 1H), 2.61-2.54 (m, 1H), 2.47-2.37 (m, 6H), 2.11-1.87 (m, 6H), |
| | | | | 1.80-1.61 (m, 2H), 1.42-1.31 (m, 2H), 1.22 (br dd, J = 2.5, 8.3 |
| | | | | Hz, 2H), 0.95 (s, 9H) |
| I-13^{e} | AD | AE | 1289.3 | 8.93 (d, J = 2.4 Hz, 1H), 8.47 (br d, J = 5.2 Hz, 1H), 7.99 (d, J = |
| | | | | 8.4 Hz, 1H), 7.75 (br d, J = 8.0 Hz, 1H), 7.64 (br d, J = 7.6 Hz, |
| | | | | 1H), 7.45-7.26 (m, 6H), 7.00-6.88 (m, 5H), 4.79 (br s, 2H), 4.56 |
| | | | | (br d, J = 8.8 Hz, 1H), 4.49 (br t, J = 7.2 Hz, 1H), 4.32 (br s, 1H), |
| | | | | 4.27-4.21 (m, 1H), 4.19-4.11 (m, 1H), 3.96 (br d, J = 5.6 Hz, |
| | | | | 4H), 3.69 (br d, J = 6.4 Hz, 2H), 3.64-3.53 (m, 2H), 3.21-3.14 |
| | | | | (m, 3H), 3.03-2.95 (m, 2H), 2.30 (br d, J = 1.6 Hz, 3H), 2.23- |
| | | | | 2.13 (m, 3H), 2.09-2.00 (m, 2H), 1.98-1.81 (m, 4H), 1.74-1.59 |
| | | | | (m, 5H), 1.53-1.42 (m, 3H), 1.37-1.30 (m, 3H), 1.22-1.13 (m, |
| | | | | 5H), 0.92 (s, 9H) |
| I-14^{e} | AD | V | 1331.3 | 13.00-12.65 (m, 1H), 8.97 (d, J = 1.6 Hz, 1H), 8.48 (t, J = 6.0 Hz, |
| | | | | 1H), 8.02 (d, J = 8.0 Hz, 1H), 7.78 (d, J = 8.0 Hz, 1H), 7.67 (br d, |
| | | | | J = 7.6 Hz, 1H), 7.49-7.43 (m, 2H), 7.39 (dd, J = 3.6, 7.6 Hz, 2H), |
| | | | | 7.35 (d, J = 7.2 Hz, 1H), 7.29 (dd, J = 2.4, 9.2 Hz, 1H), 7.07-6.92 |
| | | | | (m, 6H), 4.83 (s, 2H), 4.60 (d, J = 9.2 Hz, 1H), 4.52 (t, J = 8.0 Hz, |
| | | | | 1H), 4.35 (br s, 1H), 4.32-4.26 (m, 1H), 4.22-4.15 (m, 1H), 4.04- |
| | | | | 3.94 (m, 5H), 3.76-3.72 (m, 2H), 3.66-3.58 (m, 2H), 3.23 (br |
| | | | | dd, J = 8.4, 11.2 Hz, 4H), 3.03 (br s, 2H), 2.89 (s, 1H), 2.75 (s, 1H), |
| | | | | 2.62-2.55 (m, 1H), 2.45 (s, 3H), 2.19 (br t, J = 7.6 Hz, 1H), 2.12 |
| | | | | (br d, J = 7.6 Hz, 1H), 2.02-1.88 (m, 4H), 1.75-1.64 (m, 4H), |
| | | | | 1.47-1.31 (m, 8H), 1.23 (br dd, J = 2.4, 11.2 Hz, 8H), 0.96 (s, 9H) |
| I-15^{e} | AB | K | 1197.1 | 13.13-12.74 (m, 1H), 8.99 (s, 1H), 8.36 (br d, J = 7.6 Hz, 1H), |
| | | | | 8.04 (d, J = 7.6 Hz, 1H), 7.80 (br d, J = 8.4 Hz, 2H), 7.67 (br d, J = |
| | | | | 7.6 Hz, 1H), 7.53-7.33 (m, 9H), 7.32-7.25 (m, 1H), 7.24-7.18 |
| | | | | (m, 1H), 7.10-7.01 (m, 1H), 5.09 (br s, 1H), 4.97-4.88 (m, 1H), |
| | | | | 4.83 (s, 2H), 4.56-4.47 (m, 1H), 4.46-4.37 (m, 3H), 4.28 (br s, |
| | | | | 1H), 4.12 (br t, J = 6.0 Hz, 2H), 3.73 (br t, J = 5.6 Hz, 2H), 3.60 |
| | | | | (br s, 2H), 3.21 (br t, J = 7.6 Hz, 2H), 3.04 (br t, J = 5.6 Hz, 2H), |
| | | | | 3.00 (s, 2H), 2.88 (s, 1H), 2.46 (s, 3H), 2.32-2.11 (m, 4H), 2.06- |
| | | | | 1.93 (m, 3H), 1.83-1.64 (m, 3H), 1.37 (d, J = 7.2 Hz, 3H), 0.97- |
| | | | | 0.90 (m, 9H) |
| I-16^{e} | AB | AC | 1224.4 | 8.99 (s, 1H), 8.38 (br d, J = 6.8 Hz, 1H), 8.09-7.97 (m, 1H), 7.80 |
| | | | | (br d, J = 8.0 Hz, 2H), 7.70-7.64 (m, 1H), 7.55-7.32 (m, 9H), |
| | | | | 7.32-7.16 (m, 3H), 7.09-7.01 (m, 1H), 5.16-5.05 (m, 1H), 4.97- |
| | | | | 4.86 (m, 1H), 4.83 (br s, 2H), 4.50 (br d, J = 8.4 Hz, 1H), 4.46- |
| | | | | 4.34 (m, 3H), 4.32-4.24 (m, 1H), 4.11 (br t, J = 4.8 Hz, 2H), 3.74 |
| | | | | (br s, 2H), 3.60 (br d, J = 1.2 Hz, 2H), 3.23-3.18 (m, 2H), 3.08- |
| | | | | 2.98 (m, 4H), 2.46 (br s, 3H), 2.27-2.21 (m, 2H), 1.98 (br d, J = |
| | | | | 3.2 Hz, 5H), 1.85-1.76 (m, 1H), 1.50-1.41 (m, 4H), 1.37 (br d, J = |
| | | | | 6.4 Hz, 2H), 1.28-1.19 (m, 3H), 0.92 (br s, 9H) |
| I-17^{e} | AG | B | 1266.4 | 13.02-12.38 (m, 1H), 8.98 (s, 1H), 8.36 (d, J = 7.8 Hz, 1H), 8.03 |
| | | | | (d, J = 7.8 Hz, 1H), 7.81-7.71 (m, 2H), 7.67 (br d, J = 7.1 Hz, |
| | | | | 1H), 7.54-7.32 (m, 8H), 7.26 (td, J = 7.2, 10.7 Hz, 1H), 7.19 (br |
| | | | | d, J = 7.1 Hz, 1H), 7.10-7.00 (m, 1H), 4.91 (br t, J = 7.3 Hz, 1H), |
| | | | | 4.82 (s, 2H), 4.51 (br d, J = 9.5 Hz, 1H), 4.46-4.36 (m, 3H), 4.27 |
| | | | | (br s, 1H), 4.10 (br t, J = 6.1 Hz, 2H), 3.73 (br t, J = 5.7 Hz, 2H), |
| | | | | 3.63-3.53 (m, 2H), 3.23-3.17 (m, 2H), 3.08-2.95 (m, 4H), 2.86 |
| | | | | (s, 1H), 2.46-2.44 (m, 3H), 2.38-2.31 (m, 1H), 2.27-2.17 (m, |
| | | | | 2H), 2.12-1.91 (m, 4H), 1.79 (ddd, J = 4.9, 8.3, 12.9 Hz, 1H), 1.48- |
| | | | | 1.39 (m, 4H), 1.37 (d, J = 7.0 Hz, 2H), 1.28-1.15 (m, 10H), 0.92 |
| | | | | (s, 9H) |
| I-18^{e} | AB | E | 1256.3 | 13.47-12.14 (m, 1H), 8.99-8.86 (m, 1H), 8.52-8.39 (m, 1H), |
| | | | | 8.16 (s, 1H), 8.01 (d, J = 7.6 Hz, 1H), 7.78 (d, J = 8.0 Hz, 1H), |
| | | | | 7.66 (br d, J = 7.2 Hz, 1H), 7.51-7.33 (m, 5H), 7.32-7.21 (m, |
| | | | | 2H), 7.20-7.13 (m, 1H), 7.03 (dt, J = 5.6, 7.9 Hz, 1H), 6.99-6.89 |
| | | | | (m, 2H), 4.81 (s, 2H), 4.58 (br d, J = 9.2 Hz, 1H), 4.50 (br t, J = |
| | | | | 8.0 Hz, 1H), 4.46 (s, 1H), 4.40 (s, 1H), 4.33 (br s, 1H), 4.29-4.18 |
| | | | | (m, 2H), 4.14-3.99 (m, 4H), 3.74-3.67 (m, 2H), 3.65-3.57 (m, |
| | | | | 2H), 3.22-3.17 (m, 2H), 3.09-2.96 (m, 4H), 2.89 (s, 1H), 2.60 |
| | | | | (br d, J = 7.6 Hz, 1H), 2.52 (br s, 2H), 2.45-2.40 (m, 3H), 2.14- |
| | | | | 1.82 (m, 6H), 1.44-1.28 (m, 2H), 1.27-1.18 (m, 2H), 0.94 (s, 9H) |
| I-19^{e} | AG | AE | 1285 | 8.97-8.95 (m, 1H), 8.51-8.46 (m, 1H), 8.02 (d, J = 7.8 Hz, 1H), |
| | | | | 7.80-7.76 (m, 1H), 7.67-7.62 (m, 1H), 7.37-7.37 (m, 1H), 7.48- |
| | | | | 7.32 (m, 5H), 7.31-7.26 (m, 1H), 7.18 (br d, J = 7.4 Hz, 1H), |
| | | | | 7.06-7.00 (m, 1H), 6.96-6.90 (m, 2H), 4.81 (br s, 2H), 4.59 (d, J = |
| | | | | 9.8 Hz, 1H), 4.51 (t, J = 7.8 Hz, 1H), 4.38 (s, 1H), 4.36-4.32 |
| | | | | (m, 1H), 4.31-4.22 (m, 1H), 4.21-4.14 (m, 1H), 4.10 (t, J = 6.4 |
| | | | | Hz, 2H), 3.97 (q, J = 6.0 Hz, 2H), 3.74-3.69 (m, 2H), 3.66-3.58 |
| | | | | (m, 2H), 3.01 (s, 4H), 2.86 (s, 1H), 2.44-2.42 (m, 3H), 2.28 (br t, |
| | | | | J = 7.0 Hz, 2H), 2.10-2.05 (m, 1H), 2.00-1.89 (m, 4H), 1.73- |
| | | | | 1.68 (m, 2H), 1.55-1.48 (m, 2H), 1.45-1.30 (m, 6H), 1.24-1.19 |
| | | | | (m, 3H), 0.94 (s, 9H) |
| I-20^{e} | AG | V | 1325.49 | 8.97 (s, 1H), 8.53-8.47 (m, 1H), 7.99 (d, J = 7.4 Hz, 1H), 7.76 (d, |
| | | | | J = 8.4 Hz, 1H), 7.67 (br d, J = 6.6 Hz, 1H), 7.46-7.33 (m, 5H), |
| | | | | 7.32-7.27 (m, 2H), 7.24 (br d, J = 11.0 Hz, 1H), 7.22-7.16 (m, |
| | | | | 1H), 7.02-7.01 (m, 1H), 7.07-7.01 (m, 1H), 6.97-6.92 (m, 2H), |
| | | | | 4.83 (s, 2H), 4.61-4.57 (m, 1H), 4.54-4.48 (m, 1H), 4.41 (s, 1H), |
| | | | | 4.37 (s, 2H), 4.32-4.24 (m, 1H), 4.22-4.13 (m, 1H), 4.11-4.07 |
| | | | | (m, 2H), 4.01-3.93 (m, 2H), 3.73-3.69 (m, 2H), 3.64-3.56 (m, |
| | | | | 3H), 3.02-2.99 (m, 4H), 2.86 (s, 1H), 2.44 (s, 3H), 2.26-2.19 (m, |
| | | | | 3H), 2.13-2.04 (m, 2H), 2.01-1.92 (m, 4H), 1.75-1.65 (m, 4H), |
| | | | | 1.40-1.35 (m, 4H), 1.22 (br s, 6H), 0.95 (s, 9H) |
| I-30^{e} | A | BY | 1087.9 | 8.98 (s, 1H), 8.36 (br d, J = 7.8 Hz, 1H), 7.89-7.79 (m, 2H), 7.77- |
| | | | | 7.68 (m, 1H), 7.54-7.46 (m, 1H), 7.44-7.41 (m, 2H), 7.39-7.35 |
| | | | | (m, 2H), 7.24 (br s, 3H), 7.07-6.94 (m, 4H), 5.00 (br s, 2H), 4.95- |
| | | | | 4.88 (m, 1H), 4.50 (d, J = 9.2 Hz, 1H), 4.43-4.39 (m, 1H), 4.27 |
| | | | | (br s, 1H), 4.02 (br t, J = 6.0 Hz, 2H), 3.73 (s, 2H), 3.59 (br s, 2H), |
| | | | | 3.23-3.19 (m, 3H), 2.99 (br t, J = 5.6 Hz, 2H), 2.92-2.85 (m, |
| | | | | 1H), 2.82-2.76 (m, 1H), 2.61-2.55 (m, 1H), 2.45 (s, 3H), 2.00 |
| | | | | (br d, J = 5.6 Hz, 2H), 1.82-1.82 (m, 1H), 1.82-1.75 (m, 1H), |
| | | | | 1.36 (d, J = 6.8 Hz, 3H), 0.96 (s, 1H), 0.91 (s, 1H), 0.86 (s, 9H) |
| I-38 | C | O | 1526.3 | 8.99-8.94 (m, 1H), 8.54-8.42 (m, 1H), 8.12 (d, J = 1.6 Hz, 1H), |
| | | | | 7.95 (dd, J = 2.0, 9.2 Hz, 1H), 7.71 (d, J = 8.8 Hz, 2H), 7.44 (d, J = |
| | | | | 2.8 Hz, 1H), 7.42-7.41 (m, 1H), 7.47-7.30 (m, 1H), 7.29-7.23 |
| | | | | (m, 2H), 7.21-7.08 (m, 3H), 7.00 (br d, J = 9.2 Hz, 1H), 6.86 (br |
| | | | | d, J = 8.4 Hz, 3H), 6.05 (d, J = 10.0 Hz, 1H), 5.11 (dd, J = 3.6, 6.8 |
| | | | | Hz, 1H), 4.47-4.22 (m, 4H), 4.19-3.99 (m, 3H), 3.81-3.70 (m, |
| | | | | 1H), 3.66 (d, J = 9.6 Hz, 1H), 3.60-3.36 (m, 5H), 3.28-3.12 (m, |
| | | | | 6H), 3.10-2.71 (m, 3H), 2.67 (td, J = 2.0, 3.6 Hz, 1H), 2.48-2.42 |
| | | | | (m, 5H), 2.42-2.36 (m, 4H), 2.35-2.31 (m, 3H), 2.29-2.15 (m, |
| | | | | 6H), 2.14-1.96 (m, 5H), 1.95-1.68 (m, 5H), 1.43 (br t, J = 6.0 |
| | | | | Hz, 2H), 0.93 (s, 9H), 0.80 (dd, J = 6.8, 13.6 Hz, 3H), 0.67 (d, J = |
| | | | | 6.8 Hz, 1H), 0.57 (d, J = 6.8 Hz, 1H) |
| I-39 | C | P | 1578.5^{c} | 9.00-8.95 (m, 1H), 8.51 (t, J = 6.0 Hz, 1H), 8.11 (s, 1H), 7.96- |
| | | | | 7.92 (m, 1H), 7.71 (d, J = 8.8 Hz, 2H), 7.45-7.30 (m, 10H), 7.26 |
| | | | | (t, J = 7.6 Hz, 2H), 7.19-7.15 (m, 1H), 7.12 (d, J = 8.4 Hz, 2H), |
| | | | | 7.02-6.95 (m, 1H), 6.85 (d, J = 8.4 Hz, 3H), 6.06 (s, 1H), 5.14- |
| | | | | 5.08 (m, 1H), 4.39-4.29 (m, 4H), 4.17-4.01 (m, 4H), 3.79-3.73 |
| | | | | (m, 1H), 3.66 (d, J = 9.6 Hz, 1H), 3.59-3.53 (m, 1H), 3.44 (dd, J = |
| | | | | 5.2, 10.3 Hz, 2H), 3.24-3.10 (m, 5H), 2.46-2.43 (m, 4H), 2.23 |
| | | | | (dd, J = 2.0, 4.4 Hz, 10H), 1.99 (s, 4H), 1.92-1.88 (m, 1H), 1.72- |
| | | | | 1.59 (m, 3H), 1.53-1.39 (m, 6H), 1.37-1.19 (m, 6H), 1.05 (t, J = |
| | | | | 7.2 Hz, 1H), 0.99-0.93 (m, 10H), 0.87-0.76 (m, 4H) |
| I-40^{f} | A | BF | 929.4 | 8.99-8.95 (m, 1H), 8.39-8.28 (m, 2H), 8.02-7.96 (m, 1H), 7.89- |
| | | | | 7.67 (m, 2H), 7.64-7.56 (m, 1H), 7.49-7.26 (m, 10H), 6.74 (br |
| | | | | d, J = 8.4 Hz, 1H), 4.95-4.82 (m, 3H), 4.52-4.39 (m, 2H), 4.30- |
| | | | | 4.17 (m, 2H), 3.82 (br s, 2H), 2.99-2.91 (m, 3H), 2.84-2.70 (m, |
| | | | | 3H), 2.45 (s, 3H), 2.40-2.31 (m, 2H), 2.05-1.98 (m, 1H), 1.83- |
| | | | | 1.74 (m, 1H), 1.40-1.33 (m, 3H), 0.88 (s, 9H) |
| I-41^{f} | BP | BF | 500.7^{d} | 8.98 (s, 1H), 8.39 (br dd, J = 8.0, 13.2 Hz, 1H), 8.07-8.01 (m, |
| | | | | 1H), 7.95-7.83 (m, 1H), 7.79 (br d, J = 8.0 Hz, 1H), 7.59 (br d, J = |
| | | | | 8.0 Hz, 1H), 7.55-7.46 (m, 2H), 7.46-7.40 (m, 3H), 7.39-7.32 |
| | | | | (m, 5H), 6.96-6.90 (m, 1H), 4.92 (br s, 2H), 4.51-4.40 (m, 2H), |
| | | | | 4.28-4.23 (m, 1H), 4.14-4.02 (m, 1H), 3.99 (br d, J = 9.6 Hz, |
| | | | | 1H), 3.86 (br d, J = 2.8 Hz, 2H), 3.59-3.55 (m, 2H), 2.98 (br s, |
| | | | | 2H), 2.91 (s, 3H), 2.84 (br d, J = 7.2 Hz, 2H), 2.79-2.75 (m, 2H), |
| | | | | 2.45 (br s, 3H), 2.37 (br s, 3H), 2.05-1.97 (m, 1H), 1.81-1.73 (m, |
| | | | | 1H), 1.36 (br dd, J = 3.2, 6.8 Hz, 2H), 0.91 (br d, J = 14.8 Hz, 9H) |
| I-44^{e} | BE | AC | 618.3^{d} | 9.01-8.96 (m, 1H), 8.52 (br d, J = 8.0 Hz, 1H), 8.06 (br t, J = 5.1 |
| | | | | Hz, 1H), 7.88 (br d, J = 7.4 Hz, 1H), 7.82-7.76 (m, 2H), 7.65 (br |
| | | | | d, J = 7.4 Hz, 1H), 7.49 (s, 1H), 7.44-7.40 (m, 2H), 7.39-7.34 |
| | | | | (m, 3H), 7.29 (br d, J = 8.8 Hz, 1H), 7.25-7.18 (m, 1H), 6.92 (br |
| | | | | d, J = 8.9 Hz, 1H), 6.63 (br d, J = 8.6 Hz, 1H), 5.05-4.96 (m, 2H), |
| | | | | 4.93-4.87 (m, 1H), 4.51 (br d, J = 9.3 Hz, 1H), 4.44 (br t, J = 8.1 |
| | | | | Hz, 1H), 4.24 (br s, 1H), 4.06 (br t, J = 4.7 Hz, 2H), 3.85 (br t, J = |
| | | | | 5.1 Hz, 2H), 3.79 (br d, J = 7.1 Hz, 2H), 3.58 (br s, 2H), 2.45 (s, |
| | | | | 3H), 2.39 (br s, 2H), 2.19-2.12 (m, 5H), 1.81-1.71 (m, 3H), 1.69- |
| | | | | 1.58 (m, 4H), 1.57-1.44 (m, 8H), 1.36 (br d, J = 6.9 Hz, 3H), |
| | | | | 1.26-1.10 (m, 7H), 0.92 (s, 11H) |
| I-84 | C | E | 1576.0^{b} | 12.32-11.93 (m, 1H), 10.15-9.67 (m, 1H), 9.60-9.29 (m, 1H), |
| | | | | 8.98 (s, 1H), 8.49 (br t, J = 6.0 Hz, 1H), 8.18 (d, J = 1.6 Hz, 1H), |
| | | | | 8.08-7.92 (m, 1H), 7.77 (br d, J = 8.4 Hz, 2H), 7.45-7.36 (m, |
| | | | | 3H), 7.30-7.20 (m, 5H), 7.18-7.10 (m, 4H), 7.03-6.92 (m, 5H), |
| | | | | 4.59 (br d, J = 9.2 Hz, 1H), 4.52 (br t, J = 8.3 Hz, 1H), 4.35 (br s, |
| | | | | 1H), 4.33-4.18 (m, 2H), 4.16-3.99 (m, 4H), 3.97-3.84 (m, 2H), |
| | | | | 3.68-3.60 (m, 4H), 3.39-3.35 (m, 4H), 3.26-2.73 (m, 12H), 2.56 |
| | | | | (br d, J = 7.2 Hz, 2H), 2.45 (s, 3H), 2.28 (br s, 2H), 2.18-1.86 (m, |
| | | | | 9H), 1.48 (br t, J = 5.6 Hz, 2H), 1.41-1.28 (m, 2H), 1.22 (br d, J = |
| | | | | 8.4 Hz, 2H), 1.03-0.97 (m, 6H), 0.95 (s, 9H) |
| I-85 | C | F | 1617.5 | 12.29-12.01 (m, 1H), 9.81-9.58 (m, 1H), 9.45-9.18 (m, 1H), |
| | | | | 8.98 (s, 1H), 8.55-8.42 (m, 1H), 8.18 (d, J = 2.0 Hz, 1H), 8.00 |
| | | | | (dd, J = 1.6, 9.2 Hz, 1H), 7.77 (br d, J = 8.8 Hz, 2H), 7.45-7.37 |
| | | | | (m, 3H), 7.32-7.20 (m, 5H), 7.18-7.11 (m, 4H), 7.02-6.91 (m, |
| | | | | 5H), 4.60 (br d, J = 9.2 Hz, 1H), 4.52 (br t, J = 8.4 Hz, 1H), 4.35 |
| | | | | (br s, 1H), 4.31 (br s, 1H), 4.17 (br d, J = 5.6 Hz, 1H), 4.04 (br t, J = |
| | | | | 5.6 Hz, 4H), 3.92 (br d, J = 5.6 Hz, 1H), 3.65-3.59 (m, 4H), |
| | | | | 3.46-3.28 (m, 7H), 3.26-2.70 (m, 9H), 2.45 (s, 3H), 2.34 (br s, |
| | | | | 2H), 2.28 (br s, 2H), 2.21-2.00 (m, 6H), 1.92 (dt, J = 4.0, 8.7 Hz, |
| | | | | 1H), 1.81-1.72 (m, 2H), 1.54-1.42 (m, 6H), 1.41-1.32 (m, 4H), |
| | | | | 1.22 (br dd, J = 2.8, 8.3 Hz, 2H), 1.01 (s, 6H), 0.95 (s, 9H) |
| I-86 | C | K | 1514.2 | 8.98 (s, 1H), 8.38-8.33 (m, 1H), 8.13 (s, 1H), 7.92 (s, 1H), 7.83- |
| | | | | 7.78 (m, 1H), 7.72 (br d, J = 8.4 Hz, 2H), 7.44-7.41 (m, 2H), 7.37 |
| | | | | (d, J = 8.4 Hz, 4H), 7.34-7.31 (m, 2H), 7.26 (t, J = 7.6 Hz, 2H), |
| | | | | 7.18 (br d, J = 7.6 Hz, 1H), 7.12 (br d, J = 8.4 Hz, 2H), 6.89-6.85 |
| | | | | (m, 2H), 5.11-5.06 (m, 1H), 4.93-4.88 (m, 1H), 4.51 (d, J = 9.2 |
| | | | | Hz, 1H), 4.46-4.38 (m, 1H), 4.25 (s, 1H), 4.12-4.04 (m, 1H), |
| | | | | 3.62-3.57 (m, 2H), 3.36 (br s, 2H), 3.24-3.17 (m, 4H), 2.68- |
| | | | | 2.66 (m, 2H), 2.45 (s, 3H), 2.34-2.32 (m, 3H), 2.23 (br d, J = 7.6 |
| | | | | Hz, 10H), 2.01-1.96 (m, 6H), 1.83-1.76 (m, 2H), 1.70-1.66 (m, |
| | | | | 2H), 1.46-1.42 (m, 3H), 1.37 (d, J = 7.2 Hz, 3H), 1.26-1.20 (m, |
| | | | | 7H), 0.97 (s, 6H), 0.93 (s, 9H) |
| I-87^{e} | U | X | 619.2^{d} | 8.98 (s, 1H), 8.56-8.42 (m, 1H), 8.25 (s, 1H), 8.00 (br d, J = 7.2 |
| | | | | Hz, 1H), 7.76 (d, J = 8.4 Hz, 1H), 7.67 (br d, J = 8.4 Hz, 1H), 7.45- |
| | | | | 7.40 (m, 4H), 7.39-7.35 (m, 3H), 7.32 (br t, J = 7.7 Hz, 3H), |
| | | | | 7.19-7.16 (m, 1H), 7.12-7.08 (m, 1H), 6.07-6.02 (m, 1H), 4.84 |
| | | | | (br s, 2H), 4.41-4.30 (m, 6H), 4.14-4.10 (m, 1H), 4.08-4.03 (m, |
| | | | | 3H), 3.74-3.63 (m, 4H), 3.18-3.16 (m, 2H), 3.04-3.01 (m, 4H), |
| | | | | 2.88 (s, 1H), 2.45 (s, 3H), 2.03-1.99 (m, 3H), 1.92-1.88 (m, 3H), |
| | | | | 1.69 (br s, 3H), 1.57-1.48 (m, 3H), 1.40-1.33 (m, 3H), 0.97- |
| | | | | 0.93 (m, 3H), 0.81 (br dd, J = 6.4, 14.0 Hz, 3H) |
| I-88^{e} | U | Y | 1279.7 | 8.98 (d, J = 1.6 Hz, 1H), 8.64-8.44 (m, 1H), 7.84-7.71 (m, 2H), |
| | | | | 7.61-7.53 (m, 1H), 7.44-7.35 (m, 4H), 7.32-7.24 (m, 5H), 7.17 |
| | | | | (br d, J = 8.8 Hz, 1H), 7.12-7.06 (m, 2H), 6.09-6.00 (m, 1H), |
| | | | | 5.20-5.03 (m, 2H), 4.44-4.24 (m, 7H), 4.11-4.03 (m, 4H), 3.77- |
| | | | | 3.72 (m, 1H), 3.67-3.63 (m, 2H), 3.57 (br dd, J = 3.6, 9.6 Hz, |
| | | | | 1H), 3.22 (br d, J = 7.2 Hz, 2H), 3.03 (br d, J = 3.6 Hz, 2H), 2.99 |
| | | | | (br t, J = 5.6 Hz, 2H), 2.88 (s, 1H), 2.44 (s, 3H), 2.06-1.96 (m, |
| | | | | 4H), 1.93-1.84 (m, 2H), 1.70-1.58 (m, 3H), 1.53-1.44 (m, 3H), |
| | | | | 1.27 (br s, 2H), 1.23 (br s, 6H), 0.97-0.92 (m, 3H), 0.83-0.77 (m, |
| | | | | 3H) |
| I-89 | C | AW | 1614.3 | 9.00-8.94 (m, 1H), 8.46 (br t, J = 5.6 Hz, 1H), 8.08 (s, 1H), 7.93 |
| | | | | (br d, J = 8.8 Hz, 1H), 7.71 (d, J = 8.8 Hz, 2H), 7.41-7.33 (m, |
| | | | | 6H), 7.31-7.27 (m, 3H), 7.22-7.17 (m, 1H), 7.13-7.09 (m, 3H), |
| | | | | 6.93 (br d, J = 9.2 Hz, 2H), 6.81 (br d, J = 8.8 Hz, 3H), 5.17 (d, J = |
| | | | | 3.6 Hz, 1H), 4.60 (br d, J = 9.2 Hz, 1H), 4.53-4.47 (m, 1H), |
| | | | | 4.42-4.34 (m, 2H), 4.25-4.19 (m, 1H), 4.09-4.00 (m, 1H), 3.68- |
| | | | | 3.58 (m, 2H), 3.51-3.40 (m, 4H), 3.15 (br s, 4H), 3.10 (br d, J = |
| | | | | 7.2 Hz, 2H), 2.74 (br dd, J = 2.0, 5.6 Hz, 2H), 2.45 (s, 3H), 2.23 |
| | | | | (br d, J = 6.8 Hz, 4H), 2.14-2.07 (m, 4H), 2.01-1.94 (m, 4H), |
| | | | | 1.73-1.68 (m, 2H), 1.51 (br d, J = 12.0 Hz, 4H), 1.46-1.32 (m, |
| | | | | 6H), 1.24-1.21 (m, 2H), 1.18 (t, J = 7.2 Hz, 5H), 0.96 (s, 18H) |
| I-90 | C | AX | 1614.3 | 8.97 (s, 1H), 8.55 (br t, J = 6.0 Hz, 1H), 8.08 (s, 1H), 7.93 (dd, J = |
| | | | | 1.2, 9.6 Hz, 1H), 7.71 (d, J = 8.8 Hz, 2H), 7.43 (d, J = 8.0 Hz, 1H), |
| | | | | 7.38-7.32 (m, 5H), 7.31-7.26 (m, 3H), 7.19 (br d, J = 7.2 Hz, |
| | | | | 1H), 7.11 (d, J = 8.4 Hz, 2H), 7.00 (s, 1H), 6.90 (br d, J = 8.0 Hz, |
| | | | | 2H), 6.80 (br d, J = 8.8 Hz, 3H), 5.18 (d, J = 3.6 Hz, 1H), 4.77 (br |
| | | | | s, 1H), 4.60 (br d, J = 9.2 Hz, 1H), 4.53 (t, J = 8.0 Hz, 1H), 4.39- |
| | | | | 4.33 (m, 2H), 4.22-4.14 (m, 1H), 4.06-3.99 (m, 1H), 3.68-3.59 |
| | | | | (m, 2H), 3.44 (br s, 4H), 3.16 (br s, 4H), 3.12 (br s, 2H), 2.76 (br |
| | | | | s, 2H), 2.44 (s, 3H), 2.28 (br s, 4H), 2.22 (br s, 4H), 1.98 (br s, 5H), |
| | | | | 1.77-1.62 (m, 6H), 1.48-1.37 (m, 6H), 1.24-1.20 (m, 2H), 1.17 |
| | | | | (t, J = 7.2 Hz, 3H), 0.96 (br d, J = 1.6 Hz, 18H) |
| I-91 | C | AZ | 1587.5 | 8.97 (s, 1H), 8.48 (br t, J = 5.6 Hz, 1H), 8.09 (s, 1H), 7.93 (br d, J = |
| | | | | 9.6 Hz, 1H), 7.71 (br d, J = 8.8 Hz, 2H), 7.41 (d, J = 8.0 Hz, 1H), |
| | | | | 7.38 (s, 1H), 7.36 (s, 2H), 7.33 (s, 2H), 7.29-7.25 (m, 3H), 7.18 |
| | | | | (br d, J = 7.2 Hz, 1H), 7.11 (d, J = 8.4 Hz, 2H), 6.95 (br d, J = 6.8 |
| | | | | Hz, 2H), 6.82 (br dd, J = 2.8, 4.0 Hz, 2H), 6.72 (s, 1H), 5.16 (d, J = |
| | | | | 4.0 Hz, 1H), 4.80-4.75 (m, 1H), 4.59 (d, J = 8.8 Hz, 1H), 4.52 |
| | | | | (t, J = 8.4 Hz, 1H), 4.34 (br d, J = 5.2 Hz, 1H), 4.27 (br dd, J = 6.4, |
| | | | | 11.6 Hz, 2H), 4.08-4.03 (m, 1H), 3.68-3.56 (m, 4H), 3.48-3.37 |
| | | | | (m, 2H), 3.14 (br dd, J = 4.0, 7.2 Hz, 4H), 2.77-2.72 (m, 2H), 2.44 |
| | | | | (s, 4H), 2.23 (br d, J = 4.4 Hz, 6H), 2.13-2.04 (m, 2H), 1.98 (br s, |
| | | | | 3H), 1.96-1.89 (m, 2H), 1.76-1.68 (m, 1H), 1.43 (br t, J = 5.6 |
| | | | | Hz, 2H), 1.40-1.37 (m, 1H), 1.35-1.32 (m, 1H), 1.27-1.24 (m, |
| | | | | 6H), 1.23 (br d, J = 2.0 Hz, 2H), 1.21-1.19 (m, 1H), 1.18-1.14 |
| | | | | (m, 1H), 0.96 (s, 15H) |
| I-92 | C | BO | 1472.6 | 8.53 (t, J = 5.4 Hz, 1H), 8.13 (s, 1H), 7.96 (dd, J = 1.8, 9.2 Hz, |
| | | | | 1H), 7.72 (br d, J = 8.8 Hz, 2H), 7.37 (d, J = 8.4 Hz, 2H), 7.35- |
| | | | | 7.31 (m, 3H), 7.31-7.22 (m, 4H), 7.20-7.15 (m, 1H), 7.12 (d, J = |
| | | | | 8.4 Hz, 2H), 7.05-7.00 (m, 2H), 6.97 (d, J = 8.0 Hz, 1H), 6.86 |
| | | | | (br d, J = 8.8 Hz, 2H), 5.15 (d, J = 3.6 Hz, 1H), 4.89 (s, 2H), 4.59 |
| | | | | (d, J = 9.2 Hz, 1H), 4.48 (t, J = 8.4 Hz, 1H), 4.36-4.31 (m, 1H), |
| | | | | 4.30-4.19 (m, 2H), 4.14-4.12 (m, 1H), 3.69-3.54 (m, 3H), 3.48- |
| | | | | 3.33 (m, 9H), 3.25 (br d, J = 19.2 Hz, 8H), 2.54-2.51 (m, 3H), |
| | | | | 2.23 (br s, 3H), 2.12-2.02 (m, 2H), 1.99 (br s, 3H), 1.88 (ddd, J = |
| | | | | 4.4, 8.8, 12.8 Hz, 1H), 1.84-1.74 (m, 1H), 1.44 (br t, J = 6.4 Hz, |
| | | | | 2H), 1.41-1.32 (m, 2H), 1.24-1.19 (m, 2H), 0.98-0.93 (m, 15H) |
| I-93^{g} | BV | AC | 631.7^{d} | 8.98 (s, 1H), 8.39-8.34 (m, 2H), 7.99-7.90 (m, 1H), 7.78 (d, J = |
| | | | | 9.2 Hz, 1H), 7.71 (t, J = 8.4 Hz, 2H), 7.50-7.21 (m, 11H), 6.89- |
| | | | | 6.85 (m, 1H), 4.95-4.80 (m, 3H), 4.50 (d, J = 9.6 Hz, 1H), 4.42 |
| | | | | (t, J = 7.6 Hz, 1H), 4.27 (d, J = 1.2 Hz, 1H), 3.96 (dd, J = 1.6, 3.2 |
| | | | | Hz, 2H), 3.73-3.69 (m, 2H), 3.59 (s, 4H), 3.17 (d, J = 2.4 Hz, 2H), |
| | | | | 3.01 (d, J = 3.2 Hz, 2H), 2.45 (s, 3H), 2.30-2.19 (m, 5H), 2.16- |
| | | | | 2.08 (m, 2H), 2.04-1.88 (m, 4H), 1.84-1.68 (m, 2H), 1.54-1.41 |
| | | | | (m, 6H), 1.37 (d, J = 6.8 Hz, 3H), 1.30-1.19 (m, 4H), 0.92 (s, 9H) |
| I-94 | AS | AC | 1214.0 | 8.98 (s, 1H), 8.37 (br d, J = 8.0 Hz, 1H), 7.87 (br d, J = 7.6 Hz, |
| | | | | 1H), 7.81-7.77 (m, 1H), 7.48 (br s, 1H), 7.44-7.41 (m, 2H), 7.37 |
| | | | | (br d, J = 5.6 Hz, 2H), 7.35-7.33 (m, 1H), 7.31-7.29 (m, 1H), |
| | | | | 7.22-7.12 (m, 4H), 4.91 (quin, J = 6.8 Hz, 1H), 4.50 (br d, J = 9.2 |
| | | | | Hz, 1H), 4.41 (t, J = 8.0 Hz, 1H), 4.36-4.31 (m, 2H), 4.27 (br s, |
| | | | | 3H), 4.17-4.12 (m, 2H), 3.59 (br s, 2H), 3.01 (s, 2H), 2.89-2.84 |
| | | | | (m, 3H), 2.44 (s, 3H), 2.34 (s, 3H), 2.29-2.20 (m, 3H), 2.12 (br d, |
| | | | | J = 6.4 Hz, 3H), 2.03 (br s, 3H), 1.81-1.75 (m, 1H), 1.55-1.45 |
| | | | | (m, 5H), 1.36 (br d, J = 7.2 Hz, 3H), 1.29-1.22 (m, 4H), 0.92 (br |
| | | | | d, J = 4.1 Hz, 9H) |
| I-95^{e} | AS | B | 1254.6 | 8.97 (s, 1H), 8.37 (br d, J = 7.6 Hz, 1H), 7.87 (br d, J = 7.6 Hz, |
| | | | | 1H), 7.78 (br d, J = 9.2 Hz, 1H), 7.49 (br d, J = 3.6 Hz, 1H), 7.44- |
| | | | | 7.41 (m, 2H), 7.39-7.34 (m, 3H), 7.30 (br d, J = 10.4 Hz, 1H), |
| | | | | 7.26-7.10 (m, 4H), 4.91 (br t, J = 7.2 Hz, 1H), 4.51 (br d, J = 9.2 |
| | | | | Hz, 1H), 4.42 (t, J = 8.0 Hz, 1H), 4.36-4.31 (m, 2H), 4.27 (br s, |
| | | | | 3H), 4.14 (br t, J = 5.6 Hz, 2H), 3.59 (br s, 2H), 3.01 (s, 2H), 2.89- |
| | | | | 2.85 (m, 3H), 2.44 (s, 3H), 2.33 (s, 3H), 2.29 (br d, J = 7.2 Hz, |
| | | | | 1H), 2.24-2.20 (m, 1H), 2.12 (br dd, J = 6.0, 13.6 Hz, 3H), 2.07- |
| | | | | 1.97 (m, 4H), 1.78 (ddd, J = 4.8, 8.0, 12.4 Hz, 1H), 1.48 (br dd, J = |
| | | | | 4.8, 6.0 Hz, 5H), 1.36 (br d, J = 6.8 Hz, 3H), 1.23 (br s, 10H), |
| | | | | 0.92 (br s, 9H) |
| I-96 | BZ | CA | 1160.7 | 8.98 (s, 1H), 8.37 (d, J = 8.0 Hz, 1H), 8.10-8.06 (m, 1H), 8.00 (d, |
| | | | | J = 8.0 Hz, 1H), 7.79-7.74 (m, 2H), 7.61 (d, J = 7.6 Hz, 1H), 7.48- |
| | | | | 7.40 (m, 5H), 7.40-7.32 (m, 5H), 7.23 (s, 1H), 6.95 (d, J = 8.8 |
| | | | | Hz, 1H), 5.13-5.07 (m, 1H), 4.99 (s, 2H), 4.94-4.89 (m, 1H), |
| | | | | 4.54-4.50 (m, 1H), 4.45-4.40 (m, 1H), 4.27 (d, J = 4.0 Hz, 2H), |
| | | | | 3.89 (t, J = 5.2 Hz, 2H), 3.82 (d, J = 7.2 Hz, 2H), 3.60 (s, 2H), 3.01 |
| | | | | (s, 2H), 2.45 (s, 3H), 2.19 (dd, J = 7.2, 14.4 Hz, 2H), 2.07 (s, 1H), |
| | | | | 2.05 (s, 3H), 2.00 (d, J = 13.2 Hz, 2H), 1.78 (td, J = 4.0, 7.6 Hz, |
| | | | | 2H), 1.70-1.59 (m, 4H), 1.54 (d, J = 12.8 Hz, 2H), 1.37 (d, J = |
| | | | | 6.8 Hz, 4H), 1.26 (d, J = 6.8 Hz, 2H), 1.17-1.06 (m, 8H), 0.93 (s, |
| | | | | 9H) |
| I-97 | C | CK | 1448.8 | 0.92-0.99 (m, 15 H), 1.11-1.28 (m, 3 H), 1.32-1.47 (m, 4 H), |
| | | | | 1.76-1.84 (m, 1 H), 1.89 (ddd, J = 12.8, 8.4, 4.4 Hz, 1 H), 2.00 (br |
| | | | | s, 3 H), 2.04-2.09 (m, 1 H), 2.14-2.42 (m, 11 H), 2.77-2.97 (m, |
| | | | | 2 H), 3.19-3.27 (m, 5 H), 3.38-3.51 (m, 5 H), 3.57-3.67 (m, 2 |
| | | | | H), 4.11 (br d, J = 2.0 Hz, 1 H), 4.27 (br t, J = 5.2 Hz, 2 H), 4.33 (br |
| | | | | s, 1 H), 4.49 (t, J = 8.0 Hz, 1 H), 4.59 (d, J = 9.2 Hz, 1 H), 4.84 (s, |
| | | | | 2 H), 5.15 (d, J = 3.6 Hz, 1 H), 6.83-6.94 (m, 5 H), 7.02 (br d, J = |
| | | | | 8.8 Hz, 1 H), 7.11-7.19 (m, 4 H), 7.24-7.35 (m, 6 H), 7.38 (d, |
| | | | | J = 8.4 Hz, 2 H), 7.72 (br d, J = 8.8 Hz, 2 H), 7.96 (dd, J = 9.2, 2.0 |
| | | | | Hz, 1 H), 8.13 (s, 1 H), 8.49 (br t, J = 5.6 Hz, 1 H) |
| I-98 | C | CM | 1466.8 | 8.52-8.43 (m, 1H), 8.17-8.07 (m, 1H), 7.99-7.90 (m, 1H), 7.71 |
| | | | | (br d, J = 8.4 Hz, 2H), 7.39-7.32 (m, 4H), 7.31-7.23 (m, 4H), |
| | | | | 7.21-7.06 (m, 4H), 6.90-6.78 (m, 4H), 6.65 (dt, J = 2.4, 8.4 Hz, |
| | | | | 1H), 5.14 (d, J = 3.2 Hz, 1H), 4.88 (s, 2H), 4.58 (d, J = 9.2 Hz, 1H), |
| | | | | 4.46 (t, J = 8.0 Hz, 1H), 4.33 (br s, 1H), 4.22 (br dd, J = 5.6, 14.3 |
| | | | | Hz, 2H), 4.12-4.03 (m, 1H), 3.69-3.54 (m, 2H), 3.38 (br d, J = |
| | | | | 3.6 Hz, 5H), 3.19 (br dd, J = 3.2, 8.7 Hz, 5H), 2.97-2.70 (m, 2H), |
| | | | | 2.23 (br s, 5H), 2.10-1.93 (m, 6H), 1.91-1.71 (m, 3H), 1.45- |
| | | | | 1.29 (m, 5H), 1.26-1.18 (m, 3H), 0.98-0.92 (m, 15H) |
| I-99^{e} | CQ | K | 1152.7 | 8.98 (s, 1H), 8.44-8.32 (m, 1H), 7.88 (br d, J = 4.0 Hz, 1H), 7.78 |
| | | | | (br d, J = 9.2 Hz, 1H), 7.57 (br d, J = 7.6 Hz, 2H), 7.50 (br s, 1H), |
| | | | | 7.44-7.35 (m, 7H), 7.21 (br t, J = 7.2 Hz, 1H), 7.17-7.10 (m, |
| | | | | 1H), 5.20-5.04 (m, 1H), 4.93-4.87 (m, 1H), 4.49 (br d, J = 9.6 |
| | | | | Hz, 1H), 4.44-4.40 (m, 1H), 4.32 (br s, 2H), 4.26 (br s, 1H), 3.88 |
| | | | | (br d, J = 7.2 Hz, 2H), 3.68 (br d, J = 5.6 Hz, 2H), 3.59 (br s, 2H), |
| | | | | 2.90 (s, 2H), 2.81 (s, 1H), 2.45 (s, 3H), 2.36 (br s, 4H), 2.19 (s, |
| | | | | 3H), 2.08-2.00 (m, 2H), 1.79 (td, J = 8.0, 12.4 Hz, 2H), 1.70- |
| | | | | 1.59 (m, 6H), 1.54 (br d, J = 12.8 Hz, 2H), 1.36 (br d, J = 6.8 Hz, |
| | | | | 3H), 1.15 (br d, J = 8.0 Hz, 4H), 0.98 (br d, J = 11.2 Hz, 2H), 0.90 |
| | | | | (br d, J = 5.6 Hz, 9H) |
| I-100^{e} | CQ | AC | 1181.2 | 8.98 (s, 1H), 8.37 (dd, J = 3.2, 8.0 Hz, 1H), 7.92-7.83 (m, 1H), |
| | | | | 7.75 (br dd, J = 2.8, 9.2 Hz, 1H), 7.58 (br d, J = 8.4 Hz, 2H), 7.50 |
| | | | | (br d, J = 8.8 Hz, 1H), 7.44-7.40 (m, 3H), 7.39-7.35 (m, 4H), |
| | | | | 7.21 (br t, J = 7.6 Hz, 1H), 7.12 (br t, J = 8.8 Hz, 1H), 5.16-5.05 |
| | | | | (m, 1H), 4.91 (br t, J = 7.2 Hz, 1H), 4.49 (dd, J = 4.8, 9.6 Hz, 1H), |
| | | | | 4.41 (br t, J = 8.0 Hz, 1H), 4.32 (br d, J = 4.0 Hz, 2H), 4.26 (br s, |
| | | | | 1H), 3.88 (br d, J = 7.2 Hz, 2H), 3.68 (br dd, J = 5.6, 11.6 Hz, 2H), |
| | | | | 3.59 (br s, 2H), 2.91 (s, 2H), 2.80 (s, 1H), 2.45 (s, 3H), 2.36 (br s, |
| | | | | 3H), 2.19 (s, 3H), 2.11-2.06 (m, 1H), 2.04-1.96 (m, 3H), 1.82- |
| | | | | 1.76 (m, 2H), 1.70-1.63 (m, 3H), 1.58-1.51 (m, 3H), 1.45 (br d, |
| | | | | J = 7.6 Hz, 2H), 1.36 (br d, J = 6.8 Hz, 4H), 1.21-1.13 (m, 6H), |
| | | | | 0.98 (br d, J = 11.2 Hz, 2H), 0.91 (s, 5H), 0.90 (br s, 4H) |
| I-101^{e} | CQ | B | 1222.7 | 8.98 (s, 1H), 8.37 (d, J = 7.9 Hz, 1H), 7.87 (dt, J = 2.3, 4.7 Hz, |
| | | | | 1H), 7.75 (br dd, J = 9.3, 12.8 Hz, 1H), 7.59 (br dd, J = 4.5, 8.4 |
| | | | | Hz, 2H), 7.51 (br d, J = 11.1 Hz, 1H), 7.45-7.34 (m, 7H), 7.21 (br |
| | | | | t, J = 7.6 Hz, 1H), 7.17-7.10 (m, 1H), 5.09 (br d, J = 1.9 Hz, 1H), |
| | | | | 4.91 (br t, J = 7.3 Hz, 1H), 4.50 (dd, J = 1.8, 9.4 Hz, 1H), 4.41 (t, |
| | | | | J = 8.1 Hz, 1H), 4.33 (br d, J = 1.4 Hz, 2H), 4.27 (br s, 1H), 3.89 |
| | | | | (br d, J = 7.1 Hz, 2H), 3.73-3.65 (m, 2H), 3.59 (br s, 2H), 2.93- |
| | | | | 2.86 (m, 2H), 2.79 (s, 1H), 2.45 (s, 3H), 2.36 (br s, 2H), 2.26-2.21 |
| | | | | (m, 1H), 2.18 (s, 3H), 2.10-1.94 (m, 4H), 1.83-1.75 (m, 2H), |
| | | | | 1.72-1.58 (m, 4H), 1.54 (br d, J = 12.8 Hz, 2H), 1.44-1.40 (m, |
| | | | | 2H), 1.36 (br d, J = 7.0 Hz, 4H), 1.32-1.27 (m, 2H), 1.23 (s, 2H), |
| | | | | 1.14 (br s, 8H), 1.03-0.97 (m, 2H), 0.92 (br d, J = 2.6 Hz, 9H) |
| I-102^{g} | CQ | W | 1212.7 | 8.94 (d, J = 14.0 Hz, 1H), 8.60-8.44 (m, 1H), 8.17 (s, 1H), 7.93- |
| | | | | 7.75 (m, 1H), 7.65-7.46 (m, 3H), 7.43-7.34 (m, 3H), 7.28 (br d, |
| | | | | J = 8.4 Hz, 1H), 7.23-7.08 (m, 2H), 6.99-6.88 (m, 2H), 5.31- |
| | | | | 5.08 (m, 1H), 4.59 (br d, J = 9.2 Hz, 1H), 4.51 (br t, J = 7.6 Hz, |
| | | | | 1H), 4.40-4.28 (m, 3H), 4.27-4.15 (m, 2H), 4.09-3.97 (m, 2H), |
| | | | | 3.87 (br d, J = 7.2 Hz, 2H), 3.77-3.57 (m, 4H), 2.98-2.82 (m, |
| | | | | 3H), 2.42 (d, J = 11.2 Hz, 3H), 2.36-2.30 (m, 4H), 2.16 (d, J = |
| | | | | 14.4 Hz, 3H), 2.10-1.96 (m, 2H), 1.94-1.78 (m, 3H), 1.71-1.60 |
| | | | | (m, 3H), 1.54 (br d, J = 11.6 Hz, 2H), 1.41-1.31 (m, 2H), 1.27- |
| | | | | 1.12 (m, 6H), 0.94 (br d, J = 3.2 Hz, 11H) |
| I-103^{e} | CQ | AE | 1240.6 | 8.97 (d, J = 5.2 Hz, 1H), 8.48 (t, J = 5.9 Hz, 1H), 7.90-7.76 (m, |
| | | | | 1H), 7.58 (d, J = 8.6 Hz, 1H), 7.50 (d, J = 5.6 Hz, 1H), 7.41-7.26 |
| | | | | (m, 4H), 7.22-7.10 (m, 2H), 6.95-6.86 (m, 2H), 5.28-5.07 (m, |
| | | | | 1H), 4.58 (d, J = 9.6 Hz, 1H), 4.51 (t, J = 8.2 Hz, 1H), 4.39-4.23 |
| | | | | (m, 4H), 4.21-4.12 (m, 1H), 3.94 (t, J = 6.4 Hz, 2H), 3.86 (dd, J = |
| | | | | 4.4, 6.8 Hz, 2H), 3.75-3.56 (m, 4H), 2.92-2.79 (m, 3H), 2.43 |
| | | | | (d, J = 13.2 Hz, 3H), 2.36-2.33 (m, 3H), 2.17 (d, J = 7.2 Hz, 3H), |
| | | | | 2.10-2.03 (m, 2H), 1.95-1.75 (m, 2H), 1.73-1.48 (m, 9H), 1.45- |
| | | | | 1.31 (m, 5H), 1.26-1.10 (m, 6H), 0.94 (s, 11H) |
| I-104^{e} | CQ | V | 1283.5 | 9.01-8.95 (m, 1H), 8.52-8.45 (m, 1H), 7.94-7.75 (m, 1H), 7.59 |
| | | | | (dd, J = 4.4, 8.4 Hz, 1H), 7.51 (d, J = 10.4 Hz, 1H), 7.42-7.31 (m, |
| | | | | 3H), 7.30-7.25 (m, 1H), 7.22-7.12 (m, 2H), 6.98-6.90 (m, 2H), |
| | | | | 5.16 (d, J = 3.2 Hz, 1H), 4.59 (d, J = 9.6 Hz, 1H), 4.55-4.46 (m, |
| | | | | 1H), 4.39-4.24 (m, 4H), 4.22-4.12 (m, 1H), 3.99-3.94 (m, 1H), |
| | | | | 3.93-3.83 (m, 3H), 3.75-3.57 (m, 4H), 2.93-2.77 (m, 3H), 2.45 |
| | | | | (d, J = 3.2 Hz, 3H), 2.36 (s, 3H), 2.22 (d, J = 6.4 Hz, 1H), 2.18 (s, |
| | | | | 3H), 2.14-2.03 (m, 2H), 2.02-1.86 (m, 3H), 1.85-1.78 (m, 1H), |
| | | | | 1.73-1.49 (m, 8H), 1.42-1.30 (m, 5H), 1.24-1.14 (m, 10H), 1.02- |
| | | | | 0.98 (m, 1H), 0.95 (s, 9H) |
| I-105 | A | CT | 1127.4 | 8.98 (s, 1H), 8.40 (d, J = 8.0 Hz, 1H), 8.21 (s, 2H), 8.04 (d, J = 8.0 |
| | | | | Hz, 1H), 7.84-7.77 (m, 2H), 7.62 (d, J = 7.2 Hz, 1H), 7.51-7.47 |
| | | | | (m, 1H), 7.45-7.41 (m, 3H), 7.40-7.36 (m, 5H), 6.75 (br d, J = |
| | | | | 8.0 Hz, 1H), 4.94-4.85 (m, 3H), 4.52 (d, J = 9.6 Hz, 1H), 4.47- |
| | | | | 4.41 (m, 1H), 4.31-4.25 (m, 1H), 3.93 (br t, J = 5.6 Hz, 2H), 3.71 |
| | | | | (s, 3H), 3.61 (br s, 2H), 3.00 (br t, J = 5.2 Hz, 2H), 2.46 (s, 3H), |
| | | | | 2.25-2.07 (m, 6H), 2.00 (br dd, J = 3.6, 7.8 Hz, 1H), 1.82-1.74 |
| | | | | (m, 1H), 1.48-1.40 (m, 4H), 1.37 (d, J = 7.2 Hz, 3H), 1.22-1.15 |
| | | | | (m, 2H), 0.94 (s, 9H) |
| I-106 | A | CU | 1042.4 | 9.00-8.97 (m, 1H), 8.37 (d, J = 7.6 Hz, 1H), 7.84-7.72 (m, 3H), |
| | | | | 7.48-7.41 (m, 2H), 7.38 (d, J = 8.4 Hz, 4H), 7.12 (d, J = 8.4 Hz, |
| | | | | 2H), 6.94-6.87 (m, 2H), 5.10 (d, J = 3.4 Hz, 1H), 4.96-4.87 (m, |
| | | | | 1H), 4.50 (d, J = 9.2 Hz, 1H), 4.42 (t, J = 8.0 Hz, 1H), 4.28 (d, J = |
| | | | | 2.0 Hz, 1H), 3.65-3.54 (m, 2H), 3.41 (dd, J = 4.8, 12.8 Hz, 2H), |
| | | | | 3.26 (s, 4H), 2.76 (s, 2H), 2.46 (s, 4H), 2.30-2.21 (m, 7H), 2.12 |
| | | | | (dd, J = 7.2, 14.4 Hz, 1H), 2.00 (s, 3H), 1.79 (ddd, J = 4.4, 8.4, 12.8 |
| | | | | Hz, 1H), 1.67 (td, J = 7.2, 14.8 Hz, 2H), 1.54-1.40 (m, 5H), 1.39- |
| | | | | 1.35 (m, 4H), 0.97 (s, 6H), 0.93 (s, 9H) |
| I-107 | A | CW | 1069.5 | 11.67-11.40 (m, 1H), 8.98 (s, 1H), 8.35 (d, J = 7.8 Hz, 1H), 7.84- |
| | | | | 7.73 (m, 3H), 7.45-7.41 (m, 2H), 7.37 (d, J = 8.6 Hz, 4H), 7.12 |
| | | | | (d, J = 8.4 Hz, 2H), 6.91 (d, J = 9.0 Hz, 2H), 5.08 (d, J = 3.6 Hz, |
| | | | | 1H), 4.97-4.86 (m, 1H), 4.50 (d, J = 9.4 Hz, 1H), 4.41 (t, J = 8.0 |
| | | | | Hz, 1H), 4.27 (br d, J = 2.3 Hz, 1H), 3.64-3.55 (m, 2H), 3.47- |
| | | | | 3.41 (m, 2H), 3.26 (br s, 4H), 2.79-2.72 (m, 2H), 2.45 (s, 3H), |
| | | | | 2.30-2.19 (m, 7H), 2.13-1.98 (m, 4H), 1.79 (m, J = 12.8 Hz, 1H), |
| | | | | 1.68-1.60 (m, 2H), 1.50-1.40 (m, 4H), 1.39-1.33 (m, 5H), 1.23 |
| | | | | (br s, 4H), 0.97 (s, 6H), 0.92 (s, 9H) |
| I-108 | CY | AC | 1539.4 | 13.03-12.84 (m, 1H), 9.46 (ddd, J = 3.6, 7.0, 14.0 Hz, 1H), 8.98 |
| | | | | (s, 1H), 8.35 (d, J = 8.0 Hz, 1H), 8.19 (d, J = 1.6 Hz, 1H), 8.04- |
| | | | | 7.99 (m, 2H), 7.82-7.75 (m, 2H), 7.72-7.67 (m, 2H), 7.50-7.42 |
| | | | | (m, 4H), 7.40-7.34 (m, 4H), 7.32-7.29 (m, 2H), 7.25 (t, J = 7.6 |
| | | | | Hz, 2H), 7.20-7.16 (m, 1H), 7.12 (br d, J = 8.8 Hz, 1H), 7.00 (br |
| | | | | d, J = 8.6 Hz, 1H), 5.11-5.04 (m, 1H), 4.94-4.89 (m, 1H), 4.88- |
| | | | | 4.83 (m, 2H), 4.53-4.48 (m, 1H), 4.43-4.38 (m, 1H), 4.27 (br s, |
| | | | | 1H), 4.10 (br dd, J = 7.4, 12.2 Hz, 1H), 3.87 (br t, J = 5.6 Hz, 2H), |
| | | | | 3.62-3.56 (m, 2H), 3.06 (br d, J = 6.4 Hz, 2H), 2.90-2.81 (m, |
| | | | | 2H), 2.52 (d, J = 1.8 Hz, 7H), 2.45 (br s, 3H), 2.26-2.21 (m, 2H), |
| | | | | 2.15-2.06 (m, 4H), 2.04-1.98 (m, 2H), 1.85-1.74 (m, 2H), 1.50- |
| | | | | 1.44 (m, 4H), 1.37 (d, J = 7.0 Hz, 3H), 1.35-1.34 (m, 1H), 1.26- |
| | | | | 1.23 (m, 2H), 0.93 (s, 9H) |
| I-109ⁱ | DU | K | 1246.3 | 8.98 (s, 1H), 8.37 (br d, J = 7.6 Hz, 1H), 8.18 (s, 1H), 8.03 (br d, J = |
| | | | | 7.6 Hz, 1H), 7.87-7.68 (m, 2H), 7.52-7.24 (m, 8H), 6.97 (br s, |
| | | | | 1H), 6.84 (br d, J = 8.8 Hz, 1H), 6.80-6.73 (m, 1H), 5.29-5.03 |
| | | | | (m, 1H), 4.94-4.87 (m, 1H), 4.78 (br s, 2H), 4.50 (br dd, J = 4.0, |
| | | | | 9.0 Hz, 1H), 4.41 (br t, J = 8.0 Hz, 1H), 4.26 (br s, 1H), 3.84 (br d, |
| | | | | J = 7.0 Hz, 4H), 3.59 (br s, 2H), 3.51-3.44 (m, 4H), 3.00-2.85 |
| | | | | (m, 8H), 2.45 (s, 3H), 2.28-2.15 (m, 4H), 2.11 (s, 3H), 2.08-1.96 |
| | | | | (m, 2H), 1.81-1.73 (m, 2H), 1.72-1.57 (m, 6H), 1.52 (br d, J = |
| | | | | 12.0 Hz, 3H), 1.40-1.31 (m, 3H), 1.14 (br d, J = 7.2 Hz, 2H), 0.91 |
| | | | | (br d, J = 5.6 Hz, 9H) |
| I-110ⁱ | DU | AC | 1261.6 | 12.97-12.48 (m, 1H), 8.98 (s, 1H), 8.37 (br d, J = 7.6 Hz, 1H), |
| | | | | 8.16 (s, 1H), 8.03 (d, J = 7.6 Hz, 1H), 7.78 (br d, J = 7.6 Hz, 2H), |
| | | | | 7.51-7.41 (m, 4H), 7.40-7.31 (m, 3H), 7.27 (s, 1H), 6.97 (br s, |
| | | | | 1H), 6.86 (br d, J = 8.8 Hz, 1H), 6.80-6.70 (m, 1H), 5.22-5.03 |
| | | | | (m, 1H), 4.98-4.85 (m, 1H), 4.84-4.76 (m, 2H), 4.50 (br dd, J = |
| | | | | 5.4, 9.2 Hz, 1H), 4.42 (br t, J = 8.0 Hz, 1H), 4.27 (br s, 1H), 3.84 |
| | | | | (br d, J = 7.2 Hz, 4H), 3.59 (br s, 2H), 3.52-3.44 (m, 4H), 2.97- |
| | | | | 2.86 (m, 8H), 2.45 (s, 3H), 2.28-2.13 (m, 4H), 2.10 (s, 3H), 2.05- |
| | | | | 1.95 (m, 2H), 1.83-1.76 (m, 2H), 1.70-1.57 (m, 4H), 1.52 (br |
| | | | | d, J = 12.0Hz, 2H), 1.47-1.40 (m, 3H), 1.37 (br d, J = 6.8 Hz, 3H), |
| | | | | 1.22-1.09 (m, 6H), 0.91 (br d, J = 6.8 Hz, 9H) |
| I-111ⁱ | DU | B | 1303.6 | 13.26-12.45 (m, 1H), 8.98 (s, 1H), 8.37 (br d, J = 7.6 Hz, 1H), |
| | | | | 8.16 (s, 1H), 8.03 (d, J = 8.0 Hz, 1H), 7.78 (br d, J = 8.4 Hz, 2H), |
| | | | | 7.49-7.40 (m, 4H), 7.39-7.31 (m, 3H), 7.27 (s, 1H), 7.00-6.94 |
| | | | | (m, 1H), 6.91-6.82 (m, 1H), 6.74 (br d, J = 11.6 Hz, 1H), 5.17- |
| | | | | 5.03 (m, 1H), 4.91 (br t, J = 7.2 Hz, 1H), 4.83-4.74 (m, 2H), 4.51 |
| | | | | (br dd, J = 2.8, 9.2 Hz, 1H), 4.42 (br t, J = 8.0 Hz, 1H), 4.27 (br s, |
| | | | | 1H), 3.89-3.81 (m, 4H), 3.59 (br s, 2H), 3.52-3.42 (m, 4H), 2.99- |
| | | | | 2.84 (m, 8H), 2.45 (s, 3H), 2.27-2.13 (m, 4H), 2.10 (s, 3H), 2.05- |
| | | | | 1.96 (m, 2H), 1.78 (dt, J = 3.6, 8.0 Hz, 2H), 1.63 (br d, J = 15.2 |
| | | | | Hz, 4H), 1.52 (br d, J = 12.0 Hz, 2H), 1.44 (br s, 4H), 1.37 (br d, J = |
| | | | | 6.9 Hz, 3H), 1.15 (br s, 10H), 0.92 (br d, J = 3.2Hz, 9H) |
| I-112 | C | DW | 1560.4 | 8.96 (s, 1H), 8.58 (br d, J = 8.0 Hz, 1H), 8.12 (s, 1H), 7.94 (br d, J = |
| | | | | 9.2 Hz, 1H), 7.71 (br d, J = 8.8 Hz, 2H), 7.52 (d, J = 8.0 Hz, 1H), |
| | | | | 7.37 (br d, J = 8.0 Hz, 2H), 7.35-7.31 (m, 2H), 7.29-7.23 (m, |
| | | | | 3H), 7.17 (br d, J = 7.6 Hz, 1H), 7.12 (br d, J = 8.4 Hz, 2H), 7.04- |
| | | | | 6.98 (m, 1H), 6.96-6.91 (m, 2H), 6.85 (br d, J = 9.2 Hz, 2H), |
| | | | | 5.14 (d, J = 3.6 Hz, 1H), 5.05-4.95 (m, 2H), 4.91-4.83 (m, 1H), |
| | | | | 4.58-4.48 (m, 2H), 4.34 (br d, J = 1.6 Hz, 1H), 4.14-4.02 (m, |
| | | | | 1H), 3.64-3.54 (m, 2H), 3.49-3.34 (m, 4H), 3.23-3.01 (m, 5H), |
| | | | | 2.81-2.70 (m, 1H), 2.42 (s, 8H), 2.23 (br s, 6H), 2.10-2.03 (m, |
| | | | | 2H), 1.99 (br s, 5H), 1.80-1.73 (m, 1H), 1.43 (br t, J = 6.4 Hz, |
| | | | | 2H), 1.35 (br d, J = 6.8 Hz, 5H), 1.26-1.14 (m, 3H), 0.97 (s, 6H), |
| | | | | 0.88 (s, 9H) |
| I-113^{g} | A | EB | 1067.8 | 9.04-8.91 (m, 1H), 8.36 (d, J = 8.0 Hz, 1H), 8.13 (s, 1H), 7.97- |
| | | | | 7.90 (m, 1H), 7.88 (d, J = 7.2 Hz, 1H), 7.68-7.50 (m, 3H), 7.47- |
| | | | | 7.32 (m, 6H), 7.28-7.16 (m, 2H), 4.95-4.87 (m, 1H), 4.52 (d, J = |
| | | | | 9.2 Hz, 1H), 4.42 (t, J = 8.0 Hz, 1H), 4.27 (s, 1H), 4.20-4.10 |
| | | | | (m, 2H), 3.89 (d, J = 7.2 Hz, 2H), 3.61 (s, 2H), 2.45 (s, 3H), 2.41- |
| | | | | 2.34 (m, 4H), 2.28 (d, J = 7.6 Hz, 1H), 2.18 (s, 3H), 2.04-1.96 (m, |
| | | | | 1H), 1.95-1.86 (m, 2H), 1.86-1.74 (m, 2H), 1.71-1.59 (m, 3H), |
| | | | | 1.54 (br d, J = 11.6 Hz, 2H), 1.36 (d, J = 6.8 Hz, 3H), 1.28-1.07 |
| | | | | (m, 4H), 1.05-0.90 (m, 11H) |
| I-195 | A | JD | 1300.1 | 12.87 (s, 1H), 11.78 (s, 1H), 8.98 (s, 1H), 8.38 (d, J = 8.0 Hz, 1H), |
| | | | | 8.03 (d, J = 7.6 Hz, 1H), 7.86 (d, J = 9.2 Hz, 1H), 7.79 (d, J = 7.6 |
| | | | | Hz, 1H), 7.62 (d, J = 8.4 Hz, 2H), 7.52-7.41 (m, 5H), 7.40-7.32 |
| | | | | (m, 4H), 7.14 (s, 1H), 6.99 (d, J = 8.8 Hz, 1H), 6.92 (d, J = 8.8 Hz, |
| | | | | 2H), 5.10 (d, J = 3.2 Hz, 1H), 5.00-4.87 (m, 3H), 4.51 (d, J = 9.2 |
| | | | | Hz, 1H), 4.47-4.40 (m, 1H), 4.28 (s, 1H), 3.97-3.85 (m, 4H), |
| | | | | 3.74 (d, J = 7.2 Hz, 2H), 3.65-3.53 (m, 2H), 3.30 (s, 2H), 3.06- |
| | | | | 2.98 (m, 2H), 2.92-2.76 (m, 2H), 2.66-2.60 (m, 1H), 2.45 (s, |
| | | | | 3H), 2.05-1.96 (m, 1H), 1.83-1.75 (m, 5H), 1.72-1.58 (m, 6H), |
| | | | | 1.50 (d, J = 12.0 Hz, 2H), 1.38 (d, J = 7.2 Hz, 3H), 1.15 (d, J = 7.2 |
| | | | | Hz, 2H), 0.98-0.88 (m, 11H) |
| I-304 | A | EL | 1182.4 | 12.85 (br s, 1H), 11.76 (s, 1H), 8.98 (s, 1H), 8.37 (d, J = 7.6 Hz, |
| | | | | 1H), 8.03 (d, J = 8.0 Hz, 1H), 7.92 (br d, J = 9.2 Hz, 1H), 7.79 (br |
| | | | | d, J = 8.0 Hz, 1H), 7.63 (br d, J = 6.4 Hz, 1H), 7.53 (d, J = 8.8 Hz, |
| | | | | 1H), 7.49-7.42 (m, 4H), 7.39-7.33 (m, 4H), 7.27 (s, 1H), 7.00 |
| | | | | (d, J = 9.2 Hz, 1H), 4.96-4.89 (m, 3H), 4.51 (d, J = 9.2 Hz, 1H), |
| | | | | 4.43 (t, J = 8.0 Hz, 1H), 4.28 (br s, 1H), 3.94 (br t, J = 5.2 Hz, 2H), |
| | | | | 3.85 (br d, J = 7.2 Hz, 2H), 3.61 (br s, 2H), 3.31 (br s, 3H), 3.03 |
| | | | | (br t, J = 5.6 Hz, 2H), 2.45 (s, 3H), 2.38 (br d, J = 8.0 Hz, 1H), 2.30- |
| | | | | 2.25 (m, 1H), 2.10 (s, 3H), 2.03-1.98 (m, 1H), 1.87-1.77 (m, |
| | | | | 4H), 1.66 (br d, J = 7.2 Hz, 2H), 1.62-1.58 (m, 1H), 1.56-1.51 |
| | | | | (m, 2H), 1.44 (br d, J = 7.2 Hz, 1H), 1.37 (d, J = 6.8 Hz, 3H), 1.23 |
| | | | | (br s, 1H), 1.14 (br d, J = 7.2 Hz, 3H), 0.93 (s, 9H) |
| I-319 | EO | EM | 1118.2 | 12.90-12.83 (m, 1H), 12.09 (s, 1H), 8.53 (t, J = 6.0 Hz, 1H), 8.00 |
| | | | | (dd, J = 8.4, 16.4 Hz, 2H), 7.82-7.74 (m, 3H), 7.64 (br d, J = 8.0 |
| | | | | Hz, 1H), 7.49-7.42 (m, 3H), 7.40-7.30 (m, 8H), 7.11-7.09 (m, |
| | | | | 1H), 7.00 (d, J = 9.2 Hz, 1H), 4.96 (s, 2H), 4.54 (d, J = 9.6 Hz, 1H), |
| | | | | 4.44-4.37 (m, 2H), 4.34 (br s, 1H), 4.20-4.10 (m, 2H), 3.93 (br |
| | | | | t, J = 6.0 Hz, 2H), 3.72 (d, J = 7.6 Hz, 2H), 3.65 (br s, 2H), 3.02 |
| | | | | (br t, J = 5.6 Hz, 2H), 2.93-2.80 (m, 2H), 2.66-2.53 (m, 1H), 2.08- |
| | | | | 1.93 (m, 3H), 1.87 (ddd, J = 4.0, 8.8, 12.8 Hz, 1H), 1.78 (s, 3H), |
| | | | | 1.23 (s, 1H), 0.88-0.84 (m, 9H), 0.81 (d, J = 6.8 Hz, 6H) |
| I-352^{b} | EO | FG | 563.0 | 13.20-12.78 (m, 1H), 11.95-11.53 (m, 1H), 8.56-8.50 (m, 2H), |
| | | | | 8.17-8.13 (m, 1H), 7.86 (br d, J = 9.6 Hz, 1H), 7.64 (br d, J = 7.6 |
| | | | | Hz, 1H), 7.52 (dd, J = 4.8, 8.0 Hz, 2H), 7.46-7.43 (m, 1H), 7.40- |
| | | | | 7.34 (m, 3H), 7.33-7.30 (m, 2H), 7.28 (br s, 1H), 7.07-6.93 (m, |
| | | | | 1H), 5.12 (br d, J = 3.2 Hz, 1H), 4.95 (br s, 2H), 4.53 (d, J = 9.4 |
| | | | | Hz, 1H), 4.43-4.37 (m, 2H), 4.33 (br s, 1H), 4.17 (br dd, J = 5.6, |
| | | | | 15.9 Hz, 1H), 4.12 (s, 1H), 3.96-3.91 (m, 2H), 3.84 (br d, J = 7.2 |
| | | | | Hz, 2H), 3.68-3.61 (m, 2H), 3.02 (br t, J = 5.6 Hz, 2H), 2.28- |
| | | | | 2.20 (m, 1H), 2.11 (s, 3H), 2.08-1.97 (m, 2H), 1.90-1.83 (m, |
| | | | | 1H), 1.78 (dt, J = 3.2, 7.2 Hz, 1H), 1.67-1.50 (m, 8H), 1.46 (br d, |
| | | | | J = 8.0 Hz, 2H), 1.37-1.20 (m, 3H), 1.14 (br d, J = 8.0 Hz, 3H), |
| | | | | 0.97 (br s, 2H), 0.92 (s, 9H) |
| I-354 | FN | CR | 1154.3 | 12.96-12.77 (m, 1H), 11.74 (s, 1H), 8.52 (br t, J = 5.7 Hz, 1H), |
| | | | | 8.03 (d, J = 8.0 Hz, 1H), 7.91 (br d, J = 9.2 Hz, 1H), 7.79 (br d, J = |
| | | | | 8.0 Hz, 1H), 7.63 (br d, J = 7.2 Hz, 1H), 7.52 (br d, J = 8.8 Hz, |
| | | | | 1H), 7.50-7.46 (m, 1H), 7.44 (br d, J = 8.4 Hz, 1H), 7.40-7.34 |
| | | | | (m, 4H), 7.33-7.30 (m, 2H), 7.26 (br s, 1H), 6.99 (br d, J = 8.8 |
| | | | | Hz, 1H), 5.10 (d, J = 3.2 Hz, 1H), 4.95 (br s, 2H), 4.59 (br d, J = |
| | | | | 9.2 Hz, 1H), 4.38 (br d, J = 8.0 Hz, 2H), 4.32 (br d, J = 4.0 Hz, |
| | | | | 2H), 4.22-4.15 (m, 1H), 4.12 (s, 1H), 3.94 (br t, J = 5.3 Hz, 2H), |
| | | | | 3.85 (br d, J = 7.2 Hz, 2H), 3.64 (br s, 2H), 3.44 (br d, J = 4.4 Hz, |
| | | | | 2H), 3.26 (br s, 2H), 3.02 (br s, 2H), 2.22 (br d, J = 7.2 Hz, 1H), |
| | | | | 2.11 (s, 4H), 2.01 (br s, 1H), 1.90-1.83 (m, 1H), 1.82-1.75 (m, |
| | | | | 1H), 1.65 (br s, 2H), 1.60 (br d, J = 6.0 Hz, 2H), 1.53 (br d, J = |
| | | | | 14.0 Hz, 3H), 1.50-1.39 (m, 4H), 1.31 (br d, J = 8.0 Hz, 2H), 1.24 |
| | | | | (br s, 1H), 1.14 (br d, J = 8.0 Hz, 3H), 0.96 (s, 4H), 0.92 (s, 3H) |
| I-355^{b} | FO | CR | 577.4 | 12.85 (br s, 1H), 11.72 (br s, 1H), 8.20-8.13 (m, 1H), 8.00 (dd, J = |
| | | | | 8.0, 12.8 Hz, 2H), 7.79 (br d, J = 8.0 Hz, 1H), 7.63 (br d, J = 7.2 |
| | | | | Hz, 1H), 7.52 (br d, J = 8.8 Hz, 1H), 7.47 (s, 1H), 7.44 (br d, J = |
| | | | | 8.4 Hz, 1H), 7.40-7.36 (m, 3H), 7.35 (s, 1H), 7.26 (s, 1H), 7.22 |
| | | | | (d, J = 8.0 Hz, 2H), 6.98 (br d, J = 8.8 Hz, 1H), 5.13 (d, J = 4.0 Hz, |
| | | | | 1H), 4.94 (br s, 2H), 4.46 (br d, J = 8.0 Hz, 1H), 4.34 (br d, J = 6.8 |
| | | | | Hz, 2H), 4.32 (br s, 1H), 4.27 (br d, J = 6.4 Hz, 1H), 4.18 (br s, |
| | | | | 1H), 4.13-4.07 (m, 1H), 3.96-3.91 (m, 2H), 3.84 (br d, J = 7.2 |
| | | | | Hz, 2H), 3.82-3.77 (m, 1H), 3.46 (br d, J = 4.8 Hz, 3H), 3.25 (br |
| | | | | s, 2H), 3.02 (br s, 2H), 2.14 (br d, J = 6.8 Hz, 1H), 2.10 (s, 3H), |
| | | | | 2.04-1.97 (m, 2H), 1.95-1.89 (m, 1H), 1.78 (br s, 1H), 1.64 (br |
| | | | | s, 2H), 1.61-1.58 (m, 1H), 1.58-1.44 (m, 7H), 1.40-1.30 (m, |
| | | | | 2H), 1.29-1.22 (m, 2H), 1.13 (br d, J = 8.0 Hz, 3H), 0.96 (s, 7H) |
| I-356 | A | FQ | 1224.9 | 12.09-11.73 (m, 1H), 9.08-8.98 (m, 1H), 8.38 (br d, J = 8.0 Hz, |
| | | | | 1H), 7.97-7.86 (m, 2H), 7.84-7.75 (m, 2H), 7.59-7.53 (m, 1H), |
| | | | | 7.49 (br d, J = 8.4 Hz, 1H), 7.42 (br d, J = 8.8 Hz, 4H), 7.39-7.35 |
| | | | | (m, 2H), 7.26 (dt, J = 3.6, 7.4 Hz, 1H), 4.92-4.87 (m, 1H), 4.51 |
| | | | | (br s, 3H), 4.48 (br s, 3H), 4.41 (br d, J = 8.0 Hz, 3H), 3.78-3.74 |
| | | | | (m, 2H), 3.70-3.67 (m, 3H), 3.58 (br s, 2H), 3.33 (br d, J = 8.0 |
| | | | | Hz, 1H), 2.46 (d, J = 2.0 Hz, 3H), 2.43 (s, 3H), 2.21 (s, 3H), 2.16 |
| | | | | (s, 1H), 2.09-2.00 (m, 2H), 1.94 (br s, 3H), 1.79 (dt, J = 4.0, 8.4 |
| | | | | Hz, 1H), 1.65 (br s, 3H), 1.56 (br s, 8H), 1.44 (br d, J = 7.2 Hz, |
| | | | | 2H), 1.38-1.35 (m, 3H), 1.32 (br d, J = 7.2 Hz, 1H), 0.94-0.90 |
| | | | | (m, 9H) |
| I-359 | EP | ET | 1271.6 | 12.95-12.80 (m, 1H), 11.80-11.67 (m, 1H), 8.98 (s, 1H), 8.58 (br |
| | | | | t, J = 6.0 Hz, 1H), 8.21 (br d, J = 8.8 Hz, 1H), 8.03 (d, J = 8.0 Hz, |
| | | | | 1H), 7.82-7.76 (m, 1H), 7.63 (br d, J = 6.8 Hz, 1H), 7.58 (br d, J = |
| | | | | 8.8 Hz, 2H), 7.49-7.46 (m, 1H), 7.46-7.43 (m, 2H), 7.43-7.39 |
| | | | | (m, 3H), 7.38-7.35 (m, 2H), 7.35-7.32 (m, 1H), 7.18-7.12 (m, |
| | | | | 1H), 7.02-6.93 (m, 1H), 6.51-6.42 (m, 2H), 5.15 (d, J = 3.6 Hz, |
| | | | | 1H), 4.98-4.92 (m, 2H), 4.61 (d, J = 9.6 Hz, 1H), 4.48-4.41 (m, |
| | | | | 2H), 4.39-4.32 (m, 1H), 4.26-4.17 (m, 1H), 3.91 (br t, J = 5.6 |
| | | | | Hz, 2H), 3.74 (br d, J = 7.2 Hz, 2H), 3.70-3.62 (m, 2H), 3.35 (br |
| | | | | d, J = 4.8 Hz, 3H), 3.30 (s, 3H), 3.01 (br t, J = 5.6 Hz, 2H), 2.44 (s, |
| | | | | 3H), 2.30-2.20 (m, 2H), 2.08-1.99 (m, 2H), 1.91 (td, J = 4.4, 8.8 |
| | | | | Hz, 1H), 1.86 (br s, 2H), 1.78-1.58 (m, 5H), 1.55-1.47 (m, 2H), |
| | | | | 1.18-1.11 (m, 3H), 0.97 (s, 9H) |
| I-360 | EP | IR | 1312.2 | 12.97-12.76 (m, 1H), 12.37-11.98 (m, 1H), 8.98 (s, 1H), 8.58 (br |
| | | | | t, J = 6.0 Hz, 1H), 8.03 (d, J = 8.0 Hz, 1H), 7.94 (br d, J = 9.6 Hz, |
| | | | | 2H), 7.79 (br d, J = 8.0 Hz, 2H), 7.62 (br d, J = 7.2 Hz, 1H), 7.50- |
| | | | | 7.32 (m, 10H), 7.12-6.85 (m, 2H), 5.15 (d, J = 3.4 Hz, 1H), 4.91 |
| | | | | (br s, 1H), 4.56 (d, J = 9.2 Hz, 1H), 4.49-4.39 (m, 2H), 4.35 (br s, |
| | | | | 1H), 4.26-4.17 (m, 1H), 3.95-3.86 (m, 2H), 3.75 (br d, J = 7.2 |
| | | | | Hz, 2H), 3.69-3.64 (m, 2H), 3.22 (br s, 3H), 3.00 (br s, 2H), 2.66- |
| | | | | 2.54 (m, 1H), 2.44 (s, 3H), 2.09-1.98 (m, 2H), 1.90 (ddd, J = |
| | | | | 4.4, 8.6, 12.6 Hz, 4H), 1.79-1.70 (m, 4H), 1.70-1.58 (m, 4H), |
| | | | | 1.50 (br d, J = 11.2 Hz, 2H), 1.25-1.23 (m, 3H), 1.19-1.12 (m, |
| | | | | 3H), 0.95 (s, 9H) |
| I-361 | A | FA | 1273.7 | 12.87 (br d, J = 2.4 Hz, 1H), 11.85-11.70 (m, 1H), 8.98 (s, 1H), |
| | | | | 8.38 (d, J = 7.6 Hz, 1H), 8.03 (d, J = 8.0 Hz, 1H), 7.86 (br d, J = |
| | | | | 9.2 Hz, 1H), 7.79 (br d, J = 8.0 Hz, 1H), 7.66-7.59 (m, 3H), 7.49- |
| | | | | 7.42 (m, 5H), 7.39-7.32 (m, 4H), 7.17 (s, 1H), 7.01-6.95 (m, |
| | | | | 1H), 6.91 (br d, J = 8.4 Hz, 2H), 5.10 (d, J = 3.2 Hz, 1H), 4.98- |
| | | | | 4.89 (m, 3H), 4.51 (d, J = 9.2 Hz, 1H), 4.43 (s, 1H), 4.31-4.25 (m, |
| | | | | 1H), 3.95-3.89 (m, 3H), 3.71 (s, 2H), 3.64-3.55 (m, 2H), 3.02 |
| | | | | (br t, J = 5.6 Hz, 2H), 2.88-2.78 (m, 2H), 2.61 (br s, 2H), 2.45 (s, |
| | | | | 3H), 2.01 (br d, J = 2.4 Hz, 1H), 1.83-1.78 (m, 4H), 1.67 (br s, |
| | | | | 2H), 1.61-1.51 (m, 2H), 1.38 (d, J = 7.2 Hz, 3H), 0.96-0.93 (m, |
| | | | | 9H), 0.88 (s, 9H) |
| I-363 | A | FG | 1211.3 | 13.08-12.89 (m, 1H), 11.73 (br s, 1H), 8.98 (s, 1H), 8.51 (dd, J = |
| | | | | 1.2, 4.8 Hz, 1H), 8.36 (d, J = 8.0 Hz, 1H), 8.15 (dd, J = 1.2, 8.4 Hz, |
| | | | | 1H), 7.80 (d, J = 9.2 Hz, 1H), 7.64 (br d, J = 8.0 Hz, 1H), 7.52 (dd, |
| | | | | J = 4.8, 8.0 Hz, 2H), 7.43 (d, J = 8.4 Hz, 3H), 7.40-7.36 (m, 3H), |
| | | | | 7.26 (s, 1H), 7.06-6.98 (m, 1H), 5.09 (d, J = 3.6 Hz, 1H), 4.96 (br |
| | | | | s, 2H), 4.94-4.89 (m, 1H), 4.51 (d, J = 9.2 Hz, 1H), 4.42 (t, J = |
| | | | | 8.4 Hz, 1H), 4.27 (br d, J = 1.6 Hz, 1H), 3.94 (brt, J = 6.0 Hz, 2H), |
| | | | | 3.85 (br d, J = 7.2 Hz, 2H), 3.60 (br s, 2H), 3.30 (br s, 3H), 3.03 |
| | | | | (br t, J = 6.0 Hz, 2H), 2.45 (s, 3H), 2.25-2.19 (m, 1H), 2.11 (s, |
| | | | | 2H), 2.04-1.98 (m, 1H), 1.79 (dt, J = 4.4, 8.4 Hz, 2H), 1.68-1.58 |
| | | | | (m, 6H), 1.55-1.51 (m, 2H), 1.48-1.43 (m, 2H), 1.37 (d, J = 7.2 |
| | | | | Hz, 3H), 1.31 (br d, J = 7.2 Hz, 2H), 1.14 (br d, J = 8.8 Hz, 3H), |
| | | | | 0.97 (br s, 2H), 0.92 (s, 9H) |
| I-364 | FH | FG | 1138.3 | 13.10-12.76 (m, 1H), 11.85-11.64 (m, 1H), 8.51 (dd, J = 1.6, 4.8 |
| | | | | Hz, 1H), 8.35 (d, J = 7.6 Hz, 1H), 8.17-8.13 (m, 1H), 7.79 (d, J = |
| | | | | 9.2 Hz, 1H), 7.64 (br d, J = 7.2 Hz, 1H), 7.52 (dd, J = 4.8, 8.4 Hz, |
| | | | | 2H), 7.46-7.44 (m, 1H), 7.42-7.37 (m, 3H), 7.28 (br d, J = 8.4 |
| | | | | Hz, 3H), 7.06-6.94 (m, 1H), 5.09 (d, J = 3.2 Hz, 1H), 4.96 (br s, |
| | | | | 2H), 4.90-4.83 (m, 1H), 4.50 (d, J = 9.2 Hz, 1H), 4.40 (t, J = 8.0 |
| | | | | Hz, 1H), 4.29-4.23 (m, 1H), 4.12 (s, 1H), 3.96-3.91 (m, 2H), |
| | | | | 3.85 (br d, J = 7.2 Hz, 2H), 3.59 (br d, J = 2.8 Hz, 2H), 3.29 (br s, |
| | | | | 3H), 3.04-3.00 (m, 2H), 2.25 (br s, 1H), 2.11 (s, 2H), 2.00-1.95 |
| | | | | (m, 1H), 1.76 (td, J = 4.4, 13.3 Hz, 3H), 1.69-1.58 (m, 6H), 1.56- |
| | | | | 1.49 (m, 3H), 1.47-1.41 (m, 2H), 1.32 (br d, J = 6.8 Hz, 3H), |
| | | | | 1.17-1.11 (m, 3H), 1.01-0.94 (m, 2H), 0.91 (s, 9H) |
| I-367 | EP | FQ | 1210.1 | 11.91-11.65 (m, 1H), 8.99-8.95 (m, 1H), 8.56 (br d, J = 6.8 Hz, |
| | | | | 1H), 7.86 (br d, J = 8.8 Hz, 2H), 7.72-7.61 (m, 2H), 7.39 (q, J = |
| | | | | 8.4 Hz, 8H), 7.24-7.19 (m, 1H), 5.12 (d, J = 3.6 Hz, 1H), 4.53 (d, |
| | | | | J = 9.2 Hz, 1H), 4.45-4.40 (m, 2H), 4.34 (br d, J = 2.0 Hz, 1H), |
| | | | | 4.23-4.17 (m, 1H), 3.74 (br s, 2H), 3.64 (br s, 5H), 3.31 (s, 3H), |
| | | | | 2.47-2.46 (m, 3H), 2.43 (s, 3H), 2.20 (br s, 5H), 2.08 (br d, J = |
| | | | | 7.2 Hz, 3H), 1.93 (br s, 4H), 1.63 (br s, 3H), 1.58 (br s, 3H), 1.54 |
| | | | | (br s, 6H), 1.44 (br d, J = 6.2 Hz, 2H), 1.31-1.25 (m, 2H), 0.92 (s, |
| | | | | 9H) |
| I-368 | FH | FR | 1137.8 | 11.99-11.64 (m, 1H), 11.44-10.80 (m, 1H), 8.37 (d, J = 8.0 Hz, |
| | | | | 1H), 8.13 (s, 1H), 7.86 (s, 1H), 7.83 (d, J = 9.2 Hz, 1H), 7.78 (s, |
| | | | | 1H), 7.68 (d, J = 8.0 Hz, 1H), 7.63-7.47 (m, 1H), 7.43-7.36 (m, |
| | | | | 4H), 7.34 (d, J = 9.2 Hz, 1H), 7.28 (d, J = 8.4 Hz, 2H), 7.25-7.19 |
| | | | | (m, 1H), 5.10 (d, J = 3.2 Hz, 1H), 4.86 (quin, J = 7.2 Hz, 1H), 4.49 |
| | | | | (d, J = 9.2 Hz, 1H), 4.39 (t, J = 8.0 Hz, 1H), 4.25 (s, 1H), 4.13 (s, |
| | | | | 1H), 3.75 (s, 2H), 3.65 (s, 3H), 3.61-3.54 (m, 2H), 3.31 (s, 2H), |
| | | | | 2.39 (s, 3H), 2.29-2.23 (m, 1H), 2.18 (s, 3H), 2.14-2.07 (m, 1H), |
| | | | | 2.00-1.96 (m, 1H), 1.93 (s, 3H), 1.73 (td, J = 4.0, 8.4 Hz, 1H), 1.67- |
| | | | | 1.60 (m, 5H), 1.58 (s, 3H), 1.54 (s, 8H), 1.32 (d, J = 7.2 Hz, 3H), |
| | | | | 0.90 (s, 9H) |
| I-369 | EO | FR | 1124.1 | 11.91-11.66 (m, 1H), 11.25-10.83 (m, 1H), 8.54 (t, J = 6.0 Hz, |
| | | | | 1H), 7.97-7.85 (m, 2H), 7.79 (d, J = 1.2 Hz, 1H), 7.68 (d, J = 8.4 |
| | | | | Hz, 1H), 7.42-7.38 (m, 1H), 7.36 (d, J = 7.6 Hz, 4H), 7.33-7.30 |
| | | | | (m, 2H), 7.26-7.17 (m, 1H), 5.11 (d, J = 3.2 Hz, 1H), 4.52 (d, J = |
| | | | | 9.6 Hz, 1H), 4.44-4.36 (m, 2H), 4.33 (s, 1H), 4.16 (dd, J = 5.6, |
| | | | | 16.0 Hz, 1H), 4.12 (s, 1H), 3.75 (s, 2H), 3.65 (s, 3H), 3.62 (s, 1H), |
| | | | | 3.39-3.36 (m, 1H), 2.40 (s, 3H), 2.27 (d, J = 7.2 Hz, 1H), 2.18 (s, |
| | | | | 3H), 2.12 (d, J = 6.8 Hz, 1H), 1.99 (d, J = 10.8 Hz, 1H), 1.94 (s, |
| | | | | 3H), 1.86 (s, 1H), 1.64 (s, 6H), 1.60-1.51 (m, 12H), 1.23 (s, 1H), |
| | | | | 0.91 (s, 9H) |
| I-370 | A | FS | 1197 | 8.98 (s, 1H), 8.38 (d, J = 8.0 Hz, 1H), 8.15 (s, 1H), 7.91-7.78 (m, |
| | | | | 2H), 7.75-7.68 (m, 1H), 7.61-7.35 (m, 8H), 7.20 (br t, J = 7.6 |
| | | | | Hz, 1H), 5.10 (br d, J = 2.8 Hz, 1H), 4.95-4.87 (m, 1H), 4.52- |
| | | | | 4.41 (m, 2H), 4.27 (br s, 1H), 3.73 (s, 2H), 3.65-3.57 (m, 5H), |
| | | | | 2.45 (s, 3H), 2.36 (br s, 3H), 2.23 (s, 5H), 2.05-1.88 (m, 5H), 1.83- |
| | | | | 1.73 (m, 3H), 1.69-1.58 (m, 6H), 1.57-1.44 (m, 8H), 1.36 (d, J = |
| | | | | 7.2 Hz, 3H), 1.29-1.19 (m, 1H), 0.92 (s, 9H) |
| I-372 | A | FR | 1211.3 | 11.90-11.69 (m, 1H), 11.13-10.90 (m, 1H), 9.03-8.95 (m, 1H), |
| | | | | 8.37 (d, J = 7.6 Hz, 1H), 7.85-7.78 (m, 2H), 7.69 (d, J = 8.8 Hz, |
| | | | | 1H), 7.45-7.42 (m, 2H), 7.38 (d, J = 6.4 Hz, 4H), 7.34 (s, 1H), |
| | | | | 7.22 (d, J = 4.8 Hz, 1H), 5.10 (d, J = 2.8 Hz, 1H), 4.96-4.87 (m, |
| | | | | 1H), 4.51 (d, J = 9.2 Hz, 1H), 4.43 (t, J = 7.8 Hz, 1H), 4.27 (s, 1H), |
| | | | | 3.76 (s, 2H), 3.66 (s, 3H), 3.60 (d, J = 7.0 Hz, 2H), 3.39-3.37 (m, |
| | | | | 1H), 2.46 (s, 3H), 2.40 (s, 3H), 2.25 (s, 1H), 2.19 (s, 3H), 2.13 (d, |
| | | | | J = 6.8 Hz, 1H), 1.99 (d, J = 10.1 Hz, 1H), 1.94 (s, 3H), 1.79 (ddd, |
| | | | | J = 4.4, 8.4, 12.4 Hz, 1H), 1.64 (s, 6H), 1.60-1.54 (m, 11H), 1.37 |
| | | | | (d, J = 6.9 Hz, 3H), 0.92 (s, 9H) |
| I-373 | A | IT | 1251.5 | 12.87 (d, J = 1.6 Hz, 1H), 9.00-8.96 (m, 1H), 8.37 (d, J = 7.8 Hz, |
| | | | | 1H), 8.03 (d, J = 8.0 Hz, 1H), 7.86 (d, J = 8.8 Hz, 1H), 7.79 (d, J = |
| | | | | 8.0 Hz, 1H), 7.62 (d, J = 7.6 Hz, 1H), 7.50-7.46 (m, 1H), 7.46- |
| | | | | 7.40 (m, 5H), 7.39-7.35 (m, 5H), 6.82 (d, J = 8.4 Hz, 1H), 5.10 |
| | | | | (d, J = 3.2 Hz, 1H), 4.94-4.88 (m, 3H), 4.48 (d, J = 9.2 Hz, 1H), |
| | | | | 4.42 (t, J = 8.0 Hz, 1H), 4.27 (s, 1H), 3.93 (t, J = 5.6 Hz, 2H), 3.84 |
| | | | | (d, J = 6.8 Hz, 2H), 3.61-3.53 (m, 2H), 3.19-3.07 (m, 3H), 3.00 |
| | | | | (t, J = 5.2 Hz, 4H), 2.45 (s, 3H), 2.14 (s, 3H), 2.05-1.97 (m, 1H), |
| | | | | 1.79 (dd, J = 3.6, 8.8 Hz, 3H), 1.70-1.60 (m, 5H), 1.58-1.50 (m, |
| | | | | 4H), 1.37 (d, J = 6.8 Hz, 3H), 1.22-1.09 (m, 4H), 1.03-0.96 (m, |
| | | | | 2H), 0.95-0.89 (m, 11H) |
| I-375^{j} | EO | JB | 1126.1 | 8.58-8.53 (m, 1H), 8.51 (dd, J = 1.2, 4.7 Hz, 1H), 8.27 (br d, J = |
| | | | | 9.2 Hz, 1H), 8.17-8.11 (m, 1H), 7.62 (d, J = 7.6 Hz, 1H), 7.52 (dd, |
| | | | | J = 4.8, 8.1 Hz, 1H), 7.45-7.40 (m, 2H), 7.40-7.34 (m, 4H), 7.33- |
| | | | | 7.30 (m, 2H), 6.73 (d, J = 8.8 Hz, 1H), 5.30-4.98 (m, 1H), 4.87 |
| | | | | (s, 2H), 4.55 (d, J = 9.6 Hz, 1H), 4.45-4.37 (m, 2H), 4.34 (br s, |
| | | | | 1H), 4.17 (br dd, J = 5.2, 15.8 Hz, 1H), 4.12 (s, 1H), 3.91 (br t, J = |
| | | | | 6.0 Hz, 2H), 3.84 (br d, J = 7.2 Hz, 2H), 3.65 (br d, J = 3.2 Hz, |
| | | | | 1H), 3.60 (br d, J = 10 Hz, 1H), 3.22 (br d, J = 5.2 Hz, 3H), 3.06- |
| | | | | 2.97 (m, 6H), 2.57 (br dd, J = 7.2, 10.8 Hz, 2H), 2.16 (s, 3H), 2.05- |
| | | | | 1.98 (m, 1H), 1.88 (dt, J = 4.4, 8.4 Hz, 1H), 1.82-1.74 (m, 1H), |
| | | | | 1.70-1.62 (m, 2H), 1.60 (br d, J = 6.8 Hz, 1H), 1.55 (br d, J = 12.0 |
| | | | | Hz, 2H), 1.15 (br d, J = 8.0 Hz, 3H), 1.01-0.97 (m, 1H), 0.93 (s, |
| | | | | 9H) |
| I-376^{k} | A | JB | 1212.4 | 8.98 (s, 1H), 8.51 (dd, J = 1.6, 4.6 Hz, 1H), 8.38 (br d, J = 8.0 Hz, |
| | | | | 1H), 8.23 (br d, J = 9.2 Hz, 1H), 8.16-8.13 (m, 1H), 7.62 (br d, J = |
| | | | | 6.8 Hz, 1H), 7.52 (dd, J = 4.8, 8.1 Hz, 1H), 7.46-7.41 (m, 4H), |
| | | | | 7.40-7.34 (m, 4H), 6.80-6.67 (m, 1H), 4.95-4.88 (m, 2H), 4.87 |
| | | | | (s, 2H), 4.52 (br d, J = 9.2 Hz, 1H), 4.46-4.39 (m, 2H), 4.28 (br s, |
| | | | | 2H), 3.94-3.89 (m, 3H), 3.84 (br d, J = 7.2 Hz, 3H), 3.60 (br s, |
| | | | | 2H), 3.23 (br s, 2H), 3.04 (br d, J = 6.0 Hz, 3H), 2.99 (br d, J = 3.6 |
| | | | | Hz, 2H), 2.45 (s, 3H), 2.16 (s, 3H), 2.04-1.97 (m, 2H), 1.81-1.77 |
| | | | | (m, 2H), 1.67-1.62 (m, 3H), 1.54 (br d, J = 11.2 Hz, 2H), 1.37 (br |
| | | | | d, J = 7.2 Hz, 3H), 1.14 (br d, J = 8.0 Hz, 2H), 0.98 (br s, 2H), 0.93 |
| | | | | (s, 9H) |
| I-377^{k} | JK | JB | 1228.8 | 8.98 (s, 1H), 8.51 (dd, J = 1.6, 4.8 Hz, 1H), 8.36 (d, J = 8.0 Hz, |
| | | | | 1H), 8.23 (d, J = 9.2 Hz, 1H), 8.15 (dd, J = 1.6, 8.0 Hz, 1H), 7.62 |
| | | | | (d, J = 7.6 Hz, 1H), 7.54-7.50 (m, 1H), 7.45-7.40 (m, 5H), 7.38 |
| | | | | (dd, J = 4.4, 8.0 Hz, 3H), 6.76-6.71 (m, 1H), 5.23-5.03 (m, 1H), |
| | | | | 4.89-4.84 (m, 3H), 4.81-4.70 (m, 1H), 4.53 (d, J = 9.2 Hz, 1H), |
| | | | | 4.47 (t, J = 8.0 Hz, 1H), 4.28 (s, 1H), 3.91 (t, J = 5.6 Hz, 2H), 3.84 |
| | | | | (d, J = 7.2 Hz, 2H), 3.65-3.56 (m, 4H), 3.23 (d, J = 6.0 Hz, 2H), |
| | | | | 3.06 (d, J = 6.4 Hz, 4H), 2.99 (t, J = 5.6 Hz, 2H), 2.62-2.55 (m, |
| | | | | 2H), 2.45 (s, 3H), 2.18-2.14 (m, 3H), 2.05-1.98 (m, 1H), 1.85- |
| | | | | 1.76 (m, 2H), 1.69-1.59 (m, 3H), 1.55 (d, J = 11.2 Hz, 2H), 1.23 |
| | | | | (s, 1H), 1.15 (d, J = 8.8 Hz, 3H), 1.01-0.98 (m, 1H), 0.94 (s, 9H) |
| I-378^{j} | IF | JB | 1156.1 | 8.51 (dd, J = 1.2, 4.8 Hz, 1H), 8.35 (br d, J = 8.4 Hz, 1H), 8.23 (br |
| | | | | d, J = 9.2 Hz, 1H), 8.17-8.13 (m, 1H), 7.62 (d, J = 7.2 Hz, 1H), |
| | | | | 7.52 (dd, J = 4.8, 8.0 Hz, 1H), 7.47-7.33 (m, 6H), 7.33-7.26 (m, |
| | | | | 2H), 6.73 (d, J = 8.4 Hz, 1H), 4.89-4.79 (m, 3H), 4.78-4.69 (m, |
| | | | | 1H), 4.52 (d, J = 9.2 Hz, 1H), 4.45 (t, J = 8.0 Hz, 1H), 4.26 (br s, |
| | | | | 1H), 4.13 (s, 1H), 3.91 (br t, J = 5.4 Hz, 2H), 3.83 (br d, J = 7.2 Hz, |
| | | | | 2H), 3.64-3.51 (m, 5H), 3.05-2.96 (m, 7H), 2.61-2.52 (m, 2H), |
| | | | | 2.16 (s, 3H), 2.03-1.95 (m, 1H), 1.82-1.72 (m, 2H), 1.70-1.49 |
| | | | | (m, 6H), 1.25-1.10 (m, 4H), 0.98 (br s, 1H), 0.93 (s, 9H) |
| I-380 | A | JJ | 1300.1 | 12.90-12.82 (m, 1H), 12.05-11.93 (m, 1H), 8.98 (s, 1H), 8.38 (d, |
| | | | | J = 8.0 Hz, 1H), 8.02 (d, J = 8.0 Hz, 1H), 7.82 (br dd, J = 8.4, 16.6 |
| | | | | Hz, 2H), 7.64 (br d, J = 7.2 Hz, 1H), 7.50-7.42 (m, 5H), 7.40- |
| | | | | 7.34 (m, 6H), 7.22-7.12 (m, 3H), 6.99 (br d, J = 6.8 Hz, 1H), 5.10 |
| | | | | (d, J = 3.6 Hz, 1H), 4.96-4.89 (m, 3H), 4.50 (d, J = 9.6Hz, 1H), |
| | | | | 4.43 (t, J = 8.0 Hz, 1H), 4.27 (br d, J = 2.4 Hz, 1H), 3.94 (br t, J = |
| | | | | 6.0 Hz, 2H), 3.76-3.70 (m, 4H), 3.62-3.55 (m, 2H), 3.02 (br s, |
| | | | | 2H), 2.73 (br s, 2H), 2.45 (s, 3H), 1.99 (br d, J = 9.6 Hz, 1H), 1.85- |
| | | | | 1.77 (m, 5H), 1.73-1.59 (m, 8H), 1.50 (br d, J = 10.4 Hz, 2H), |
| | | | | 1.37 (d, J = 7.2 Hz, 3H), 1.14 (br d, J = 7.6 Hz, 3H), 0.94 (s, 9H), |
| | | | | 0.93-0.91 (m, 2H) |
| I-381 | A | IU | 1265.7 | 8.98 (s, 1H), 8.38 (d, J = 7.9 Hz, 1H), 8.04 (d, J = 8.0 Hz, 1H), 7.88- |
| | | | | 7.74 (m, 2H), 7.64 (d, J = 7.3 Hz, 1H), 7.48-7.34 (m, 10H), 6.88- |
| | | | | 6.74 (m, 1H), 5.12 (d, J = 3.4 Hz, 1H), 4.94-4.88 (m, 3H), 4.48 |
| | | | | (d, J = 9.3 Hz, 1H), 4.40 (t, J = 7.9 Hz, 1H), 4.28 (br s, 1H), 3.90 |
| | | | | (br t, J = 5.6 Hz, 2H), 3.88-3.72 (m, 3H), 3.18-3.08 (m, 5H), 3.04- |
| | | | | 2.98 (m, 3H), 2.84-2.74 (m, 2H), 2.44 (s, 3H), 2.14 (s, 3H), 2.00 |
| | | | | (br dd, J = 1.7, 9.1 Hz, 1H), 1.88-1.72 (m, 6H), 1.72-1.58 (m, |
| | | | | 6H), 1.54 (br dd, J = 1.7, 11.8 Hz, 3H), 1.38 (d, J = 7.0 Hz, 3H), |
| | | | | 1.24-1.08 (m, 4H), 0.98 (br s, 1H), 0.94-0.88 (m, 9H) |
| I-382 | FH | FQ | 1151.8 | 8.40-8.30 (m, 1H), 7.81-7.74 (m, 2H), 7.72-7.65 (m, 1H), 7.53 |
| | | | | (br d, J = 2.0 Hz, 1H), 7.47-7.32 (m, 6H), 7.28 (d, J = 8.4 Hz, 3H), |
| | | | | 5.08 (d, J = 4.0 Hz, 1H), 4.89-4.84 (m, 1H), 4.49 (d, J = 9.2 Hz, |
| | | | | 1H), 4.42-4.38 (m, 1H), 4.25 (br d, J = 3.2 Hz, 1H), 4.11 (s, 1H), |
| | | | | 3.74 (br s, 2H), 3.69 (s, 1H), 3.63 (br s, 3H), 3.58 (br s, 2H), 2.38- |
| | | | | 2.35 (m, 3H), 2.23-2.19 (m, 3H), 1.93 (br s, 5H), 1.64 (br d, J = |
| | | | | 2.4 Hz, 4H), 1.58 (br s, 2H), 1.55 (br s, 6H), 1.50 (br d, J = 1.6 Hz, |
| | | | | 3H), 1.32 (d, J = 7.2 Hz, 3H), 1.24 (s, 6H), 0.91 (s, 9H) |
| I-410 | EP | JH | 1271.5 | 12.99-12.80 (m, 1H), 11.79-11.62 (m, 1H), 8.98 (s, 1H), 8.60- |
| | | | | 8.56 (m, 1H), 8.18 (br d, J = 9.6 Hz, 1H), 8.03 (d, J = 8.0 Hz, 1H), |
| | | | | 7.79 (br d, J = 8.0 Hz, 1H), 7.66-7.55 (m, 3H), 7.50-7.46 (m, |
| | | | | 1H), 7.44 (br d, J = 4.4 Hz, 2H), 7.41 (br d, J = 8.8 Hz, 3H), 7.39- |
| | | | | 7.36 (m, 2H), 7.35-7.33 (m, 1H), 7.14 (s, 1H), 7.01-6.91 (m, |
| | | | | 1H), 6.54-6.44 (m, 2H), 5.14 (d, J = 3.6 Hz, 1H), 4.95 (br s, 2H), |
| | | | | 4.59 (d, J = 9.2 Hz, 1H), 4.48-4.40 (m, 2H), 4.35 (br s, 1H), 4.22 |
| | | | | (br dd, J = 5.6, 16.0 Hz, 1H), 3.95-3.88 (m, 2H), 3.74 (br d, J = |
| | | | | 7.2 Hz, 2H), 3.69-3.62 (m, 2H), 3.58-3.52 (m, 1H), 3.44-3.37 |
| | | | | (m, 1H), 3.03-2.99 (m, 2H), 2.44 (s, 3H), 2.42 (br s, 1H), 2.16- |
| | | | | 2.07 (m, 3H), 2.04 (br dd, J = 4.8, 8.4 Hz, 1H), 1.95-1.88 (m, 1H), |
| | | | | 1.87-1.81 (m, 3H), 1.77-1.59 (m, 5H), 1.51 (br d, J = 11.2 Hz, |
| | | | | 2H), 1.25-1.07 (m, 5H), 0.96 (s, 9H) |
| I-411^{k} | JF | EL | — | 8.97-8.94 (m, 1H), 8.50 (br d, J = 8.0 Hz, 1H), 8.03 (d, J = 8.0 |
| | | | | Hz, 1H), 7.89 (br d, J = 8.4 Hz, 1H), 7.78 (d, J = 8.0 Hz, 1H), 7.61 |
| | | | | (br d, J = 7.6 Hz, 1H), 7.49-7.46 (m, 1H), 7.45-7.42 (m, 2H), |
| | | | | 7.41 (s, 2H), 7.38 (br s, 1H), 7.36 (br s, 2H), 7.34 (br d, J = 3.6 Hz, |
| | | | | 1H), 7.32 (br d, J = 3.6 Hz, 1H), 6.78 (br d, J = 8.0 Hz, 1H), 5.19 |
| | | | | (br s, 1H), 4.87 (br s, 1H), 4.48 (br t, J = 8.0 Hz, 1H), 4.33 (br d, J = |
| | | | | 8.4 Hz, 1H), 3.95-3.91 (m, 3H), 3.83 (br d, J = 7.2 Hz, 2H), |
| | | | | 3.75 (br d, J = 7.6 Hz, 2H), 3.08-3.03 (m, 2H), 3.00 (br d, J = 5.2 |
| | | | | Hz, 3H), 2.44 (s, 3H), 2.43 (br s, 1H), 2.42-2.37 (m, 2H), 2.29- |
| | | | | 2.21 (m, 3H), 2.13 (s, 3H), 2.00-1.91 (m, 1H), 1.81-1.73 (m, |
| | | | | 3H), 1.71 (br d, J = 8.4 Hz, 1H), 1.68-1.63 (m, 3H), 1.60 (br s, |
| | | | | 1H), 1.53 (br d, J = 12.4 Hz, 2H), 1.36 (br d, J = 6.8 Hz, 3H), 1.18- |
| | | | | 1.11 (m, 3H), 0.95 (s, 9H), 0.91 (br s, 1H) |
| I-413 | EO | GG | 1139.6 | 13.46-12.61 (m, 1H), 8.57-8.49 (m, 2H), 8.15 (dd, J = 1.4, 8.1 |
| | | | | Hz, 2H), 7.63 (d, J = 7.4 Hz, 1H), 7.52 (dd, J = 4.7, 8.2 Hz, 1H), |
| | | | | 7.49-7.45 (m, 1H), 7.44 (s, 1H), 7.40-7.35 (m, 2H), 7.35-7.33 |
| | | | | (m, 2H), 7.33-7.29 (m, 2H), 6.95-6.86 (m, 1H), 5.17-5.06 (m, |
| | | | | 1H), 4.92 (s, 2H), 4.52 (d, J = 9.4 Hz, 1H), 4.43-4.35 (m, 2H), |
| | | | | 4.33 (s, 1H), 4.17 (dd, J = 5.4, 16.1 Hz, 1H), 4.12 (s, 1H), 3.94 (t, |
| | | | | J = 5.6 Hz, 2H), 3.84 (d, J = 7.2 Hz, 2H), 3.68-3.59 (m, 2H), 3.46- |
| | | | | 3.38 (m, 4H), 3.01 (t, J = 5.2 Hz, 2H), 2.96-2.87 (m, 2H), 2.87- |
| | | | | 2.74 (m, 2H), 2.34 (s, 3H), 2.13 (s, 3H), 2.04-1.97 (m, 1H), 1.87 |
| | | | | (dt, J = 4.4, 8.4 Hz, 1H), 1.83-1.74 (m, 1H), 1.65 (d, J = 1.2 Hz, |
| | | | | 2H), 1.60 (d, J = 5.6 Hz, 1H), 1.54 (d, J = 11.6 Hz, 2H), 1.20-1.11 |
| | | | | (m, 3H), 1.02-0.95 (m, 2H), 0.92 (s, 9H) |
| I-420 | A | JA | 1247.8 | 12.85 (br s, 1H), 11.60 (s, 1H), 8.98 (s, 1H), 8.61 (d, J = 9.2 Hz, |
| | | | | 1H), 8.39 (d, J = 8.0 Hz, 1H), 8.03 (d, J = 8.0 Hz, 1H), 7.79 (br d, |
| | | | | J = 8.0 Hz, 1H), 7.63 (br d, J = 7.2 Hz, 1H), 7.49 (dd, J = 8.8, 10.8 |
| | | | | Hz, 2H), 7.45-7.42 (m, 3H), 7.38 (s, 4H), 7.28 (s, 1H), 6.99 (br d, |
| | | | | J = 8.8 Hz, 1H), 5.11 (d, J = 3.6 Hz, 1H), 4.99 (br s, 2H), 4.90 (s, |
| | | | | 1H), 4.54 (d, J = 9.2 Hz, 1H), 4.43 (t, J = 8.0 Hz, 1H), 4.27 (br s, |
| | | | | 1H), 3.90 (br d, J = 6.0 Hz, 2H), 3.87 (br s, 2H), 3.64-3.59 (m, |
| | | | | 1H), 3.57 (br s, 1H), 3.43 (br d, J = 5.2 Hz, 2H), 3.04-3.00 (m, |
| | | | | 2H), 2.94 (br d, J = 4.8 Hz, 2H), 2.67 (br s, 1H), 2.45 (s, 3H), 2.11 |
| | | | | (s, 3H), 2.00 (br d, J = 8.4 Hz, 1H), 1.82-1.77 (m, 3H), 1.65 (br s, |
| | | | | 2H), 1.62-1.59 (m, 1H), 1.53 (br s, 4H), 1.37 (d, J = 6.8 Hz, 3H), |
| | | | | 1.23 (s, 2H), 1.15 (br s, 3H), 1.01-0.98 (m, 1H), 0.94 (s, 9H) |
| I-422 | EP | IP | 1210.2 | 13.02-12.71 (m, 1H), 8.98 (s, 1H), 8.57 (t, J = 6.0 Hz, 1H), 8.26- |
| | | | | 8.16 (m, 1H), 8.04 (d, J = 7.6 Hz, 1H), 7.79 (d, J = 7.9 Hz, 1H), |
| | | | | 7.61 (d, J = 7.3 Hz, 1H), 7.50-7.45 (m, 1H), 7.44-7.39 (m, 4H), |
| | | | | 7.39-7.33 (m, 5H), 6.78 (d, J = 8.6 Hz, 1H), 5.22-5.07 (m, 1H), |
| | | | | 4.89 (s, 2H), 4.56 (d, J = 9.3 Hz, 1H), 4.42 (t, J = 7.9 Hz, 2H), 4.35 |
| | | | | (s, 1H), 4.21 (dd, J = 5.4, 15.9 Hz, 1H), 3.92 (t, J = 5.6 Hz, 3H), |
| | | | | 3.84 (d, J = 7.3 Hz, 3H), 3.73-3.60 (m, 4H), 3.55 (s, 2H), 3.18 (d, |
| | | | | J = 3.3 Hz, 3H), 3.00 (br t, J = 5.4 Hz, 3H), 2.45-2.42 (m, 3H), |
| | | | | 2.14 (s, 3H), 2.02 (br d, J = 8.0 Hz, 1H), 1.90 (dt, J = 4.6, 8.5 Hz, |
| | | | | 1H), 1.78 (d, J = 3.3 Hz, 1H), 1.65 (d, J = 7.1 Hz, 3H), 1.54 (d, J = |
| | | | | 11.6 Hz, 2H), 1.14 (d, J = 8.6 Hz, 3H), 0.97 (s, 1H), 0.93 (s, 9H) |
| I-427^{k} | A | GC | 1226.2 | 8.98 (s, 1H), 8.39 (br d, J = 8.0 Hz, 1H), 8.24 (br d, J = 9.2 Hz, |
| | | | | 1H), 7.92-7.82 (m, 1H), 7.66-7.62 (m, 1H), 7.57-7.52 (m, 2H), |
| | | | | 7.45-7.41 (m, 2H), 7.40-7.31 (m, 4H), 7.20 (brt, J = 7.6 Hz, 1H), |
| | | | | 7.11 (br d, J = 8.0 Hz, 1H), 5.14 (br s, 1H), 4.97-4.86 (m, 1H), |
| | | | | 4.53 (d, J = 9.2 Hz, 1H), 4.42 (t, J = 8.0 Hz, 1H), 4.27 (br s, 1H), |
| | | | | 3.74 (s, 2H), 3.61 (s, 3H), 3.58-3.54 (m, 1H), 3.12-3.06 (m, 3H), |
| | | | | 2.68-2.54 (m, 3H), 2.45 (s, 3H), 2.37-2.33 (m, 3H), 2.24 (s, 3H), |
| | | | | 2.05-1.97 (m, 1H), 1.94 (br s, 3H), 1.78 (ddd, J = 4.8, 8.4, 12.8 |
| | | | | Hz, 1H), 1.70-1.44 (m, 13H), 1.37 (d, J = 6.8 Hz, 3H), 0.94 (s, |
| | | | | 9H) |
| I-428 | A | GE | 1239.7 | 8.99-8.96 (m, 1H), 8.37 (d, J = 7.8 Hz, 1H), 8.17 (br d, J = 8.8 |
| | | | | Hz, 1H), 7.87 (br d, J = 5.6 Hz, 1H), 7.75-7.69 (m, 1H), 7.64 (br |
| | | | | d, J = 8.8 Hz, 1H), 7.60-7.50 (m, 1H), 7.45-7.40 (m, 3H), 7.39- |
| | | | | 7.31 (m, 3H), 7.28-7.18 (m, 2H), 5.10 (br s, 1H), 4.96-4.85 (m, |
| | | | | 1H), 4.51 (d, J = 9.2 Hz, 1H), 4.46-4.39 (m, 1H), 4.31-4.19 (m, |
| | | | | 1H), 3.75 (s, 2H), 3.68-3.52 (m, 7H), 3.45 (br s, 1H), 3.15-2.84 |
| | | | | (m, 5H), 2.63-2.52 (m, 2H), 2.45 (s, 3H), 2.38 (br s, 3H), 2.21 (s, |
| | | | | 3H), 2.05-1.97 (m, 1H), 1.94 (br s, 3H), 1.78 (ddd, J = 4.8, 8.4, |
| | | | | 12.8 Hz, 1H), 1.69-1.62 (m, 3H), 1.61-1.51 (m, 10H), 1.36 (d, J = |
| | | | | 7.2 Hz, 3H), 0.92 (s, 9H) |
| I-429^{j} | EO | GC | 1138.7 | 8.55 (br t, J = 6.4 Hz, 1H), 8.27 (br d, J = 9.6 Hz, 1H), 8.14 (s, 1H), |
| | | | | 7.92-7.80 (m, 1H), 7.65 (s, 1H), 7.56-7.52 (m, 2H), 7.40-7.34 |
| | | | | (m, 3H), 7.32-7.29 (m, 2H), 7.20 (br t, J = 7.6 Hz, 1H), 7.10 (br |
| | | | | d, J = 8.4 Hz, 1H), 5.19-5.09 (m, 1H), 4.54 (d, J = 9.2 Hz, 1H), |
| | | | | 4.43-4.37 (m, 2H), 4.33 (br s, 1H), 4.19-4.10 (m, 2H), 3.74 (s, |
| | | | | 2H), 3.69-3.64 (m, 1H), 3.61 (s, 4H), 3.27 (br s, 3H), 3.10-3.03 |
| | | | | (m, 4H), 2.63-2.54 (m, 2H), 2.35 (br s, 3H), 2.24 (s, 3H), 2.00 (br |
| | | | | d, J = 7.2 Hz, 1H), 1.94 (br s, 3H), 1.90-1.84 (m, 1H), 1.67-1.62 |
| | | | | (m, 3H), 1.60-1.54 (m, 9H), 0.93 (s, 9H), 0.90 (br s, 1H) |
| I-430 | EO | GE | 1153.2 | 8.54 (t, J = 6.0 Hz, 1H), 8.18 (br d, J = 9.6 Hz, 1H), 8.13 (s, 1H), |
| | | | | 7.91-7.82 (m, 1H), 7.72 (s, 1H), 7.62 (br d, J = 8.4 Hz, 1H), 7.46 |
| | | | | (s, 1H), 7.41-7.34 (m, 3H), 7.33-7.29 (m, 2H), 7.25-7.18 (m, |
| | | | | 2H), 5.19-5.04 (m, 1H), 4.52 (d, J = 9.2 Hz, 1H), 4.43-4.35 (m, |
| | | | | 2H), 4.33 (br s, 1H), 4.17 (dd, J = 5.4, 16.0 Hz, 1H), 4.12 (s, 1H), |
| | | | | 3.74 (s, 2H), 3.67-3.58 (m, 5H), 3.44 (br d, J = 4.8 Hz, 3H), 3.05- |
| | | | | 2.85 (m, 4H), 2.43-2.35 (m, 6H), 2.22 (s, 3H), 2.04-1.97 (m, |
| | | | | 1H), 1.94 (br s, 3H), 1.87 (ddd, J = 4.8, 8.4, 12.8 Hz, 1H), 1.68- |
| | | | | 1.62 (m, 3H), 1.61-1.52 (m, 9H), 0.92 (s, 9H) |
| I-431^{j} | IY | GC | 1200 | 8.41 (br t, J = 5.8 Hz, 1H), 8.28 (br d, J = 9.5 Hz, 1H), 7.99 (s, 1H), |
| | | | | 7.65 (br s, 1H), 7.57-7.52 (m, 2H), 7.41-7.35 (m, 1H), 7.33 (d, J = |
| | | | | 7.5 Hz, 1H), 7.25-7.17 (m, 1H), 7.15-7.08 (m, 1H), 7.02 (s, |
| | | | | 1H), 6.92 (d, J = 8.5 Hz, 1H), 5.15 (br s, 1H), 4.91-4.85 (m, 1H), |
| | | | | 4.59-4.53 (m, 1H), 4.47-4.39 (m, 1H), 4.35-4.27 (m, 2H), 4.19 |
| | | | | (br d, J = 5.5 Hz, 1H), 4.16-4.12 (m, 1H), 4.02 (t, J = 4.8 Hz, 2H), |
| | | | | 3.76-3.68 (m, 5H), 3.61 (s, 4H), 3.13-3.05 (m, 3H), 2.70-2.57 |
| | | | | (m, 4H), 2.37-2.32 (m, 3H), 2.25 (s, 3H), 2.07-1.99 (m, 1H), |
| | | | | 1.94 (br s, 3H), 1.90-1.83 (m, 1H), 1.67-1.63 (m, 3H), 1.61- |
| | | | | 1.54 (m, 10H), 0.92 (s, 9H) |
| I-432 | A | GI | 1237.9 | 11.40-10.67 (m, 1H), 8.98 (s, 1H), 8.38 (d, J = 7.8 Hz, 1H), 8.21 |
| | | | | (d, J = 7.6 Hz, 1H), 7.94-7.83 (m, 1H), 7.68 (s, 1H), 7.57 (d, J = |
| | | | | 8.4 Hz, 1H), 7.51-7.49 (m, 1H), 7.45-7.41 (m, 2H), 7.39-7.34 |
| | | | | (m, 3H), 7.20 (t, J = 7.6 Hz, 1H), 7.15 (d, J = 8.4 Hz, 1H), 5.13 (br |
| | | | | s, 1H), 4.91 (t, J = 7.2 Hz, 1H), 4.53 (d, J = 9.2 Hz, 1H), 4.42 (t, J = |
| | | | | 8.2 Hz, 1H), 4.29 (s, 1H), 4.05-3.95 (m, 2H), 3.91-3.81 (m, |
| | | | | 2H), 3.74 (s, 2H), 3.62 (s, 2H), 3.60-3.56 (m, 2H), 3.26-3.23 (m, |
| | | | | 2H), 3.17 (s, 3H), 2.45 (s, 3H), 2.36 (s, 2H), 2.23 (s, 3H), 1.99 (s, |
| | | | | 2H), 1.94 (s, 3H), 1.79 (ddd, J = 4.6, 8.5, 12.7 Hz, 2H), 1.64 (s, |
| | | | | 4H), 1.61-1.51 (m, 11H), 1.37 (d, J = 7.0 Hz, 3H), 0.93 (s, 9H) |
| I-433 | EO | GI | 1150.8 | 11.57-10.63 (m, 1H), 8.56 (t, J = 6.0 Hz, 1H), 8.31-8.24 (m, 1H), |
| | | | | 7.91-7.83 (m, 1H), 7.68 (s, 1H), 7.58 (d, J = 8.4 Hz, 1H), 7.50 (s, |
| | | | | 1H), 7.41-7.35 (m, 3H), 7.34-7.30 (m, 2H), 7.21 (t, J = 8.0 Hz, |
| | | | | 1H), 7.16 (d, J = 8.8 Hz, 1H), 5.15 (s, 1H), 4.56 (d, J = 9.2 Hz, 1H), |
| | | | | 4.45-4.38 (m, 2H), 4.35 (s, 1H), 4.19 (d, J = 5.6 Hz, 1H), 4.15- |
| | | | | 4.12 (m, 1H), 4.08-3.96 (m, 2H), 3.90-3.84 (m, 1H), 3.83-3.78 |
| | | | | (m, 1H), 3.75 (s, 2H), 3.67 (d, J = 3.6 Hz, 1H), 3.63 (s, 3H), 3.62- |
| | | | | 3.59 (m, 1H), 3.27 (s, 2H), 3.16 (s, 3H), 2.37 (s, 3H), 2.24 (s, 3H), |
| | | | | 2.02 (s, 1H), 1.95 (s, 3H), 1.90 (d, J = 13.2 Hz, 1H), 1.69-1.64 |
| | | | | (m, 3H), 1.62-1.54 (m, 10H), 0.94 (s, 9H) |
| I-434 | A | GK | 1285.7 | 11.93-11.57 (m, 1H), 11.30-10.84 (m, 1H), 8.98 (s, 1H), 8.38 (d, |
| | | | | J = 8.0 Hz, 1H), 8.18-8.11 (m, 1H), 7.94-7.84 (m, 1H), 7.83- |
| | | | | 7.78 (m, 1H), 7.62 (s, 3H), 7.44-7.37 (m, 5H), 7.30-7.19 (m, |
| | | | | 3H), 6.48-6.38 (m, 2H), 5.10 (d, J = 3.6 Hz, 1H), 4.96-4.89 (m, |
| | | | | 1H), 4.56 (d, J = 9.2 Hz, 1H), 4.43 (t, J = 8.0 Hz, 1H), 4.27 (s, 1H), |
| | | | | 4.10-4.04 (m, 1H), 4.01-3.92 (m, 2H), 3.88-3.82 (m, 1H), 3.71 |
| | | | | (d, J = 6.8 Hz, 1H), 3.66-3.60 (m, 7H), 2.45 (s, 3H), 2.42 (d, J = |
| | | | | 3.2 Hz, 2H), 2.02-1.87 (m, 8H), 1.69-1.57 (m, 7H), 1.52 (s, 6H), |
| | | | | 1.37 (d, J = 6.8 Hz, 3H), 0.96-0.92 (m, 9H) |
| I-435 | EP | GM | 1286.7 | 11.98-11.79 (m, 1H), 11.51-10.77 (m, 1H), 8.99 (s, 1H), 8.57 (br |
| | | | | t, J = 6.2 Hz, 1H), 8.19 (br d, J = 9.6 Hz, 1H), 7.94-7.83 (m, 1H), |
| | | | | 7.75 (br s, 1H), 7.71-7.57 (m, 3H), 7.41 (q, J = 8.3 Hz, 5H), 7.30 |
| | | | | (br s, 2H), 7.23 (br d, J = 7.8 Hz, 1H), 6.26 (br s, 2H), 5.12 (d, J = |
| | | | | 3.4 Hz, 1H), 4.58 (br d, J = 9.4 Hz, 1H), 4.49-4.41 (m, 2H), 4.34 |
| | | | | (br s, 1H), 4.22 (br dd, J = 5.4, 15.8 Hz, 1H), 4.08 (br t, J = 7.7 Hz, |
| | | | | 1H), 4.03-3.91 (m, 2H), 3.84 (br d, J = 5.5 Hz, 1H), 3.77-3.71 |
| | | | | (m, 1H), 3.69 (s, 2H), 3.64 (br s, 5H), 2.45 (s, 3H), 2.40 (br s, 2H), |
| | | | | 2.38 (br s, 3H), 2.02 (br s, 3H), 1.95 (br s, 4H), 1.90 (br s, 1H), |
| | | | | 1.70-1.57 (m, 7H), 1.52 (br s, 6H), 0.95 (s, 9H) |
| I-436 | A | GP | 1174.3 | 11.88-11.71 (m, 1H), 11.41-11.01 (m, 1H), 8.98 (s, 1H), 8.36 (br |
| | | | | d, J = 7.6 Hz, 2H), 7.84 (br s, 1H), 7.78 (br d, J = 9.2 Hz, 1H), 7.70 |
| | | | | (br d, J = 8.8 Hz, 1H), 7.45-7.40 (m, 4H), 7.38-7.35 (m, 4H), |
| | | | | 4.90 (br t, J = 7.2 Hz, 1H), 4.49 (d, J = 9.6 Hz, 1H), 4.42-4.38 (m, |
| | | | | 1H), 4.26 (br s, 1H), 3.89 (br d, J = 7.2 Hz, 2H), 3.65 (s, 3H), 3.58 |
| | | | | (br s, 3H), 2.44 (s, 3H), 2.40 (br s, 2H), 2.18 (s, 3H), 2.07 (br d, J = |
| | | | | 8.0 Hz, 1H), 2.02-1.97 (m, 1H), 1.81-1.75 (m, 2H), 1.64 (br s, |
| | | | | 6H), 1.55 (br d, J = 13.6 Hz, 3H), 1.43 (br d, J = 6.8 Hz, 2H), 1.36 |
| | | | | (d, J = 7.2 Hz, 3H), 1.31-1.27 (m, 2H), 1.21-1.12 (m, 4H), 1.03- |
| | | | | 0.96 (m, 3H), 0.90 (s, 9H) |
| I-437 | EP | GK | 1272.3 | 11.91-11.56 (m, 1H), 11.29-10.85 (m, 1H), 8.99 (s, 1H), 8.57 (t, |
| | | | | J = 6.0 Hz, 1H), 8.20 (d, J = 9.2 Hz, 1H), 7.98-7.84 (m, 1H), 7.84- |
| | | | | 7.78 (m, 1H), 7.62 (d, J = 8.4 Hz, 3H), 7.40 (q, J = 8.4 Hz, 5H), |
| | | | | 7.33-7.28 (m, 1H), 7.25-7.19 (m, 2H), 6.43 d, J = 8.0 Hz, 2H), |
| | | | | 5.12 (d, J = 3.2 Hz, 1H), 4.59 (d, J = 9.2 Hz, 1H), 4.47-4.40 (m, |
| | | | | 2H), 4.37-4.32 (m, 1H), 4.21 (dd, J = 5.6, 15.9 Hz, 1H), 4.08 (t, J = |
| | | | | 8.4 Hz, 1H), 4.02-3.94 (m, 2H), 3.85 (t, J = 6.4 Hz, 1H), 3.75- |
| | | | | 3.68 (m, 2H), 3.64 (d, J = 7.2 Hz, 6H), 2.45 (s, 3H), 2.41 (d, J = |
| | | | | 4.0 Hz, 2H), 2.02 (d, J = 9.2 Hz, 1H), 1.97-1.87 (m, 7H), 1.69- |
| | | | | 1.57 (m, 7H), 1.51 (s, 6H), 0.94 (s, 9H) |
| I-438 | FH | GK | 1212.5 | 11.91-11.55 (m, 1H), 11.23-10.91 (m, 1H), 8.36 (d, J = 7.6 Hz, |
| | | | | 1H), 8.14 (d, J = 9.2 Hz, 1H), 7.98-7.84 (m, 1H), 7.81 (s, 1H), |
| | | | | 7.62 (d, J = 7.6 Hz, 3H), 7.44-7.36 (m, 3H), 7.29 (d, J = 8.0 Hz, |
| | | | | 3H), 7.26-7.19 (m, 2H), 6.42 (d, J = 8.4 Hz, 2H), 5.09 (d, J = 3.6 |
| | | | | Hz, 1H), 4.88 (quin, J = 7.2 Hz, 1H), 4.55 (d, J = 9.3 Hz, 1H), 4.41 |
| | | | | (t, J = 8.0 Hz, 1H), 4.26 (d, J = 2.4 Hz, 1H), 4.12 (s, 1H), 4.10- |
| | | | | 4.05 (m, 1H), 4.01-3.92 (m, 2H), 3.88-3.82 (m, 1H), 3.72-3.68 |
| | | | | (m, 1H), 3.64 (d, J = 7.2 Hz, 5H), 3.59 (s, 2H), 2.42 (s, 2H), 2.02- |
| | | | | 1.83 (m, 8H), 1.70-1.56 (m, 7H), 1.51 (s, 6H), 1.33 (d, J = 7.2 Hz, |
| | | | | 3H), 0.96-0.90 (m, 9H) |
| I-439 | A | GX | 1226.3 | 11.79 (br s, 1H), 11.40-11.03 (m, 1H), 8.98 (s, 1H), 8.35 (br d, J = |
| | | | | 8.0 Hz, 2H), 8.13 (s, 1H), 8.07-7.88 (m, 1H), 7.84 (br s, 1H), |
| | | | | 7.78 (br d, J = 9.2 Hz, 1H), 7.71 (br d, J = 8.8 Hz, 1H), 7.45-7.40 |
| | | | | (m, 3H), 7.37 (d, J = 7.2 Hz, 4H), 5.23-4.97 (m, 1H), 4.91 (quin, |
| | | | | J = 7.2 Hz, 1H), 4.49 (d, J = 9.6 Hz, 1H), 4.41 (t, J = 8.0 Hz, 1H), |
| | | | | 4.27 (br s, 1H), 3.75 (s, 2H), 3.66 (s, 3H), 3.58 (br s, 2H), 2.46- |
| | | | | 2.42 (m, 4H), 2.40 (br s, 3H), 2.19 (s, 4H), 2.10-2.05 (m, 1H), |
| | | | | 2.03-1.98 (m, 1H), 1.94 (br s, 3H), 1.79 (dt, J = 4.4, 8.4 Hz, 1H), |
| | | | | 1.64 (br s, 5H), 1.60-1.54 (m, 9H), 1.44 (br d, J = 7.2 Hz, 2H), |
| | | | | 1.36 (d, J = 7.2 Hz, 3H), 1.31 (br d, J = 7.2 Hz, 2H), 0.91 (s, 9H) |
| I-440 | E | GK | 1199.2 | 11.91-11.59 (m, 1H), 11.25-10.88 (m, 1H), 8.56 (t, J = 6.0 Hz, |
| | | | | 1H), 8.20 (d, J = 9.2 Hz, 1H), 8.00-7.85 (m, 1H), 7.84-7.76 (m, |
| | | | | 1H), 7.61 (d, J = 8.0 Hz, 3H), 7.40-7.30 (m, 6H), 7.26-7.20 (m, |
| | | | | 2H), 6.42 (d, J = 8.8 Hz, 2H), 5.12 (d, J = 3.6 Hz, 1H), 4.58 (d, J = |
| | | | | 9.2 Hz, 1H), 4.44-4.38 (m, 2H), 4.33 (s, 1H), 4.21-4.12 (m, 2H), |
| | | | | 4.10-4.05 (m, 1H), 4.01-3.93 (m, 2H), 3.85 (t, J = 6.4 Hz, 1H), |
| | | | | 3.74-3.69 (m, 1H), 3.64 (d, J = 7.6 Hz, 7H), 2.42 (s, 2H), 2.00- |
| | | | | 1.86 (m, 8H), 1.69-1.56 (m, 7H), 1.52 (s, 6H), 0.94 (s, 9H) |
| I-441 | A | GR | 1247.5 | 12.06-11.62 (m, 1H), 7.89 (br d, J = 6.8 Hz, 1H), 7.82 (s, 1H), |
| | | | | 7.67-7.49 (m, 4H), 7.39 (t, J = 7.6 Hz, 1H), 7.30 (br d, J = 8.4 Hz, |
| | | | | 1H), 7.26-7.16 (m, 2H), 6.94 (br d, J = 9.2 Hz, 2H), 3.87 (d, J = |
| | | | | 7.6 Hz, 2H), 3.76 (br d, J = 12.4 Hz, 2H), 3.63 (s, 3H), 2.95-2.84 |
| | | | | (m, 2H), 2.43 (br s, 3H), 2.33-2.26 (m, 1H), 1.91 (s, 3H), 1.79- |
| | | | | 1.71 (m, 2H), 1.65-1.54 (m, 5H), 1.53-1.47 (m, 3H), 1.39 (s, |
| | | | | 9H), 1.27-1.20 (m, 2H) |
| I-442 | A | GS | 1196.4 | 13.03-12.66 (m, 1H), 11.87-11.69 (m, 1H), 8.98 (s, 1H), 8.38 (br |
| | | | | d, J = 7.6 Hz, 1H), 8.03 (d, J = 7.6 Hz, 1H), 7.80 (dd, J = 9.2, 11.6 |
| | | | | Hz, 2H), 7.63 (br d, J = 7.6 Hz, 1H), 7.56-7.45 (m, 2H), 7.45- |
| | | | | 7.41 (m, 3H), 7.39-7.33 (m, 4H), 7.30-7.23 (m, 1H), 7.04-6.92 |
| | | | | (m, 1H), 5.10 (d, J = 3.6 Hz, 1H), 4.98-4.86 (m, 3H), 4.51 (d, J = |
| | | | | 9.2 Hz, 1H), 4.42 (t, J = 8.0 Hz, 1H), 4.27 (br s, 1H), 3.93 (br d, J = |
| | | | | 6.8 Hz, 4H), 3.60 (br s, 1H), 3.31 (br s, 2H), 3.01 (br d, J = 5.6 |
| | | | | Hz, 2H), 2.45 (s, 3H), 2.35-2.32 (m, 1H), 2.31-2.17 (m, 2H), |
| | | | | 2.13 (s, 3H), 2.09-1.95 (m, 2H), 1.82-1.75 (m, 1H), 1.60 (br d, J = |
| | | | | 2.8 Hz, 6H), 1.51-1.42 (m, 4H), 1.37 (d, J = 6.8 Hz, 3H), 1.25 |
| | | | | (br dd, J = 6.0, 12.8 Hz, 4H), 0.92 (s, 9H) |
| I-443 | A | GT | 1158.5 | 12.31-11.61 (m, 1H), 11.48-10.68 (m, 1H), 8.98 (s, 1H), 8.36 (br |
| | | | | d, J = 7.6 Hz, 1H), 8.13 (s, 1H), 7.92-7.82 (m, 1H), 7.78 (br d, J = |
| | | | | 9.2 Hz, 2H), 7.71-7.63 (m, 1H), 7.62-7.50 (m, 1H), 7.45-7.39 |
| | | | | (m, 3H), 7.39-7.35 (m, 3H), 7.35-7.28 (m, 1H), 7.21 (br t, J = |
| | | | | 7.6 Hz, 1H), 5.09 (d, J = 3.2 Hz, 1H), 4.95-4.87 (m, 1H), 4.50 (d, |
| | | | | J = 9.2 Hz, 1H), 4.42 (t, J = 7.6 Hz, 1H), 4.27 (br s, 1H), 3.98 (br |
| | | | | d, J = 7.6 Hz, 2H), 3.64 (s, 3H), 3.59 (br s, 2H), 2.45 (s, 3H), 2.38 |
| | | | | (br s, 3H), 2.36-2.32 (m, 1H), 2.24-2.18 (m, 4H), 2.11-2.05 (m, |
| | | | | 1H), 2.03-1.97 (m, 1H), 1.78 (ddd, J = 4.8, 8.4, 12.8 Hz, 1H), 1.62 |
| | | | | (br d, J = 3.2 Hz, 7H), 1.51 (br s, 2H), 1.46-1.41 (m, 2H), 1.36 (d, |
| | | | | J = 7.6 Hz, 3H), 1.29 (br d, J = 6.8 Hz, 5H), 0.91 (s, 9H) |
| I-444 | E | GS | 1109.5 | 12.96-12.76 (m, 1H), 11.89-11.67 (m, 1H), 8.55 (brt, J = 6.0 Hz, |
| | | | | 1H), 8.03 (d, J = 8.0 Hz, 1H), 7.88 (br d, J = 9.2 Hz, 1H), 7.79 (d, |
| | | | | J = 8.4 Hz, 1H), 7.63 (br d, J = 7.6 Hz, 1H), 7.56-7.46 (m, 2H), |
| | | | | 7.44 (br d, J = 8.8 Hz, 1H), 7.39-7.34 (m, 4H), 7.33-7.30 (m, |
| | | | | 2H), 7.29-7.22 (m, 1H), 7.06-6.93 (m, 1H), 5.12 (d, J = 3.6 Hz, |
| | | | | 1H), 4.94 (br s, 1H), 4.54 (d, J = 9.2 Hz, 1H), 4.43-4.37 (m, 2H), |
| | | | | 4.33 (br s, 1H), 4.20-4.14 (m, 1H), 4.13 (s, 1H), 3.93 (br d, J = |
| | | | | 6.4 Hz, 4H), 3.64 (br s, 2H), 3.31 (br s, 2H), 3.02 (br s, 2H), 2.35- |
| | | | | 2.31 (m, 1H), 2.26-2.20 (m, 1H), 2.13 (s, 3H), 2.10-2.03 (m, |
| | | | | 1H), 2.03-1.96 (m, 1H), 1.87 (ddd, J = 4.4, 8.5, 12.6 Hz, 1H), 1.60 |
| | | | | (br d, J = 4.0 Hz, 6H), 1.54-1.38 (m, 5H), 1.26 (br dd, J = 7.2, |
| | | | | 10.9 Hz, 5H), 0.92 (s, 8H) |
| I-445 | E | GV | 1110.6 | 13.01 (s, 1H), 12.20-11.13 (m, 1H), 8.57-8.53 (m, 1H), 8.51 (dd, |
| | | | | J = 1.2, 4.8 Hz, 1H), 8.15 (dd, J = 1.2, 8.0 Hz, 1H), 7.86 (d, J = 9.4 |
| | | | | Hz, 1H), 7.63 (d, J = 7.4 Hz, 1H), 7.52 (dd, J = 4.8, 8.0 Hz, 1H), |
| | | | | 7.47 (s, 1H), 7.44 (d, J = 7.6 Hz, 1H), 7.39-7.29 (m, 6H), 6.95 |
| | | | | (dd, J = 2.4, 4.8 Hz, 1H), 5.12 (s, 1H), 4.94 (s, 2H), 4.53 (d, J = 9.2 |
| | | | | Hz, 1H), 4.43-4.37 (m, 2H), 4.33 (s, 1H), 4.18 (s, 1H), 4.12 (s, |
| | | | | 1H), 3.93 (d, J = 7.2 Hz, 4H), 3.64 (s, 1H), 3.18 (d, J = 2.0 Hz, 2H), |
| | | | | 3.04-2.99 (m, 2H), 2.35-2.29 (m, 1H), 2.23 (dd, J = 7.2, 14.2 Hz, |
| | | | | 1H), 2.13 (s, 3H), 2.04-1.97 (m, 1H), 1.87 (ddd, J = 4.4, 8.4, 12.8 |
| | | | | Hz, 1H), 1.59 (dd, J = 3.6, 7 .. 2 Hz, 6H), 1.52-1.41 (m, 4H), 1.26 |
| | | | | (td, J = 6.8, 13.6 Hz, 6H), 0.92 (s, 9H) |
| I-447 | A | GX | 1153.8 | 11.79 (br s, 1H), 11.56-10.85 (m, 1H), 8.40-8.30 (m, 2H), 8.13 |
| | | | | (s, 1H), 8.01-7.87 (m, 1H), 7.84 (br s, 1H), 7.77 (br d, J = 9.2 Hz, |
| | | | | 1H), 7.71 (br d, J = 8.8 Hz, 1H), 7.45-7.39 (m, 3H), 7.38-7.33 |
| | | | | (m, 2H), 7.28 (d, J = 8.0 Hz, 2H), 5.16-4.96 (m, 1H), 4.86 (br t, J = |
| | | | | 7.2 Hz, 1H), 4.49 (d, J = 9.2 Hz, 1H), 4.39 (t, J = 8.0 Hz, 1H), |
| | | | | 4.25 (br s, 1H), 4.12 (s, 1H), 3.75 (s, 2H), 3.66 (s, 3H), 3.57 (br s, |
| | | | | 2H), 2.40 (br s, 3H), 2.19 (s, 4H), 2.07 (s, 2H), 1.94 (br s, 4H), 1.77- |
| | | | | 1.71 (m, 1H), 1.69-1.61 (m, 5H), 1.60-1.54 (m, 9H), 1.42 (td, |
| | | | | J = 6.8, 13.6 Hz, 2H), 1.32 (br d, J = 7.2 Hz, 5H), 0.90 (s, 9H) |
| I-448 | A | HR | 1241.1 | 11.48-10.90 (m, 1H), 8.97 (s, 1H), 8.40 (br d, J = 7.6 Hz, 1H), |
| | | | | 8.02-7.75 (m, 2H), 7.74-7.62 (m, 2H), 7.44-7.38 (m, 4H), 7.38- |
| | | | | 7.27 (m, 4H), 5.09 (br s, 1H), 4.89 (quin, J = 7.2 Hz, 1H), 4.50- |
| | | | | 4.42 (m, 2H), 4.30-4.24 (m, 1H), 3.74 (s, 2H), 3.65 (s, 3H), 3.61- |
| | | | | 3.52 (m, 3H), 3.02 (br s, 2H), 2.44 (s, 3H), 2.40 (br s, 3H), 2.22- |
| | | | | 2.16 (m, 6H), 2.00 (br d, J = 10.0 Hz, 1H), 1.93 (br s, 4H), 1.84- |
| | | | | 1.74 (m, 3H), 1.68-1.61 (m, 4H), 1.60-1.53 (m, 10H), 1.35 (d, J = |
| | | | | 7.2 Hz, 3H), 0.91 (s, 9H) |
| I-449^{b} | A | HT | 634.0 | 12.01-11.72 (m, 1H), 11.35-11.03 (m, 1H), 8.98 (s, 1H), 8.45- |
| | | | | 8.29 (m, 2H), 7.93-7.58 (m, 3H), 7.48-7.39 (m, 4H), 7.38-7.31 |
| | | | | (m, 4H), 5.11 (d, J = 3.2 Hz, 1H), 4.92-4.84 (m, 1H), 4.50-4.39 |
| | | | | (m, 2H), 4.27 (br s, 1H), 3.75 (s, 2H), 3.66 (br s, 3H), 3.60-3.53 |
| | | | | (m, 2H), 2.95-2.89 (m, 1H), 2.80-2.72 (m, 2H), 2.45 (s, 3H), |
| | | | | 2.41 (br s, 3H), 2.20 (br s, 3H), 2.09-1.98 (m, 3H), 1.97-1.91 (m, |
| | | | | 4H), 1.86-1.73 (m, 4H), 1.69-1.63 (m, 4H), 1.62-1.50 (m, 12H), |
| | | | | 1.36 (br d, J = 6.8 Hz, 3H), 0.91 (br s, 9H) |
| I-450 | A | GY | 1313 | 12.05-11.53 (m, 1H), 9.04-8.94 (m, 1H), 8.38 (br d, J = 8.4 Hz, |
| | | | | 2H), 7.88-7.78 (m, 2H), 7.70-7.55 (m, 3H), 7.46-7.41 (m, 3H), |
| | | | | 7.40-7.36 (m, 2H), 7.34-7.18 (m, 2H), 7.00-6.83 (m, 2H), 5.11 |
| | | | | (d, J = 3.6 Hz, 1H), 4.97-4.88 (m, 1H), 4.50 (d, J = 9.2 Hz, 1H), |
| | | | | 4.43 (brt, J = 8.4 Hz, 1H), 4.31-4.25 (m, 1H), 3.92-3.78 (m, 2H), |
| | | | | 3.68-3.56 (m, 7H), 2.88-2.75 (m, 2H), 2.46-2.42 (m, 5H), 2.07- |
| | | | | 1.85 (m, 8H), 1.82-1.48 (m, 20H), 1.38 (br d, J = 7.2 Hz, 3H), |
| | | | | 0.93 (s, 9H) |
| I-451 | EP | GX | 1211.4 | 11.77 (br s, 1H), 11.32-11.04 (m, 1H), 8.98 (s, 1H), 8.55 (br t, J = |
| | | | | 6.0 Hz, 1H), 8.41-8.31 (m, 1H), 7.84 (br d, J = 9.2 Hz, 3H), 7.73- |
| | | | | 7.62 (m, 1H), 7.39 (q, J = 8.4 Hz, 7H), 5.11 (d, J = 3.6 Hz, 1H), |
| | | | | 4.55-4.50 (m, 1H), 4.46-4.39 (m, 2H), 4.34 (br dd, J = 2.4, 3.8 |
| | | | | Hz, 1H), 4.23-4.17 (m, 1H), 3.76-3.72 (m, 2H), 3.65 (br s, 5H), |
| | | | | 2.44 (s, 3H), 2.39 (br s, 2H), 2.22-2.18 (m, 4H), 2.12-1.99 (m, |
| | | | | 3H), 1.93 (br s, 4H), 1.67-1.57 (m, 8H), 1.55 (br s, 7H), 1.47- |
| | | | | 1.41 (m, 2H), 1.33-1.23 (m, 3H), 0.91 (s, 9H) |
| I-452^{l} | A | GZ | 1197.7 | 11.91-11.73 (m, 1H), 11.37-11.06 (m, 1H), 8.98 (s, 1H), 8.36 (br |
| | | | | d, J = 7.6 Hz, 2H), 7.90 (br d, J = 9.2 Hz, 2H), 7.84 (s, 1H), 7.72 |
| | | | | (d, J = 8.8 Hz, 1H), 7.45-7.41 (m, 3H), 7.39-7.35 (m, 4H), 4.90 |
| | | | | (br t, J = 7.2 Hz, 1H), 4.49 (d, J = 9.2 Hz, 1H), 4.42 (t, J = 8.0 Hz, |
| | | | | 1H), 4.26 (br s, 1H), 3.75 (s, 2H), 3.66 (s, 3H), 3.58 (br s, 2H), 2.45 |
| | | | | (s, 3H), 2.40 (s, 3H), 2.36 (br d, J = 7.6 Hz, 1H), 2.28 (br d, J = 7.6 |
| | | | | Hz, 1H), 2.19 (s, 3H), 2.00 (br d, J = 2.8 Hz, 1H), 1.94 (br s, 4H), |
| | | | | 1.88-1.83 (m, 2H), 1.77 (br s, 1H), 1.67-1.58 (m, 6H), 1.55 (br |
| | | | | s, 7H), 1.36 (d, J = 6.8 Hz, 3H), 0.90 (s, 9H) |
| I-453 | EO | HA | 1125.2 | 11.84-11.73 (m, 1H), 11.28-11.14 (m, 1H), 8.53 (t, J = 6.0 Hz, |
| | | | | 1H), 8.41-8.34 (m, 1H), 8.03-7.95 (m, 1H), 7.91-7.80 (m, 2H), |
| | | | | 7.73-7.67 (m, 1H), 7.47-7.40 (m, 1H), 7.36 (d, J = 8.4 Hz, 4H), |
| | | | | 7.33-7.29 (m, 2H), 5.11 (d, J = 3.6 Hz, 1H), 4.52 (d, J = 9.6 Hz, |
| | | | | 1H), 4.43-4.36 (m, 2H), 4.33 (br d, J = 2.8 Hz, 1H), 4.18 (d, J = |
| | | | | 5.6 Hz, 1H), 4.15-4.12 (m, 1H), 3.75 (s, 2H), 3.66 (s, 3H), 3.65- |
| | | | | 3.58 (m, 2H), 2.41 (br s, 3H), 2.31-2.21 (m, 2H), 2.19 (s, 3H), |
| | | | | 2.12 (br dd, J = 6.8, 13.6 Hz, 1H), 2.04-1.97 (m, 1H), 1.94 (br s, |
| | | | | 3H), 1.90-1.82 (m, 1H), 1.69-1.62 (m, 5H), 1.58 (br s, 3H), 1.55 |
| | | | | (br s, 9H), 0.90 (s, 9H) |
| I-454 | FH | HA | 1139.2 | 11.90-11.69 (m, 1H), 11.31-11.07 (m, 1H), 8.35 (br d, J = 8.0 |
| | | | | Hz, 2H), 8.03-7.93 (m, 1H), 7.81 (br d, J = 9.2 Hz, 2H), 7.70 (br |
| | | | | d, J = 8.8 Hz, 1H), 7.46-7.42 (m, 1H), 7.40 (d, J = 8.4 Hz, 2H), |
| | | | | 7.38-7.34 (m, 2H), 7.28 (d, J = 8.4 Hz, 2H), 5.09 (d, J = 3.6 Hz, |
| | | | | 1H), 4.90-4.82 (m, 1H), 4.48 (d, J = 9.2 Hz, 1H), 4.39 (t, J = 8.0 |
| | | | | Hz, 1H), 4.25 (br s, 1H), 4.11 (s, 1H), 3.75 (s, 2H), 3.66 (s, 3H), |
| | | | | 3.61-3.53 (m, 3H), 2.40 (br s, 3H), 2.26 (br dd, J = 7.2, 14.8 Hz, |
| | | | | 2H), 2.19 (s, 3H), 2.10 (td, J = 6.8, 14.0 Hz, 2H), 2.02-1.96 (m, |
| | | | | 1H), 1.93 (br s, 3H), 1.74 (ddd, J = 4.8, 8.4, 12.8 Hz, 1H), 1.67- |
| | | | | 1.63 (m, 4H), 1.58 (br s, 3H), 1.54 (br s, 8H), 1.32 (d, J = 6.8 Hz, |
| | | | | 3H), 0.90 (s, 9H) |
| I-455 | A | HC | 1218 | 11.98-11.57 (m, 1H), 9.01-8.95 (m, 1H), 8.43-8.34 (m, 1H), |
| | | | | 7.94-7.76 (m, 3H), 7.60 (br s, 4H), 7.45-7.42 (m, 2H), 7.41- |
| | | | | 7.36 (m, 3H), 7.34-7.26 (m, 2H), 7.33-7.14 (m, 1H), 7.00-6.83 |
| | | | | (m, 2H), 5.11 (d, J = 3.2 Hz, 1H), 4.97-4.88 (m, 1H), 4.50 (br d, |
| | | | | J = 9.2 Hz, 1H), 4.43 (br t, J = 8.0 Hz, 1H), 4.28 (br s, 1H), 3.92- |
| | | | | 3.81 (m, 2H), 3.76 (br s, 2H), 3.63 (br s, 3H), 3.61-3.54 (m, 2H), |
| | | | | 2.78 (br d, J = 2.4 Hz, 2H), 2.54 (br s, 1H), 2.45 (s, 3H), 2.41 (br |
| | | | | s, 2H), 2.08-1.87 (m, 4H), 1.87-1.67 (m, 3H), 1.65-1.51 (m, |
| | | | | 3H), 1.42-1.34 (m, 3H), 0.94 (s, 9H), 0.91 (br s, 9H) |
| I-456 | A | HE | 1236.5 | 11.91-11.76 (m, 1H), 11.41-11.08 (m, 1H), 8.98 (s, 1H), 8.39 (br |
| | | | | d, J = 8.0 Hz, 2H), 8.05-7.93 (m, 1H), 7.89 (br s, 1H), 7.84 (br d, |
| | | | | J = 9.2 Hz, 1H), 7.66 (br d, J = 8.8 Hz, 1H), 7.62 (br d, J = 9.2 Hz, |
| | | | | 2H), 7.46-7.42 (m, 3H), 7.40-7.36 (m, 2H), 7.34 (br d, J = 8.8 |
| | | | | Hz, 1H), 7.27 (s, 1H), 6.98-6.92 (m, 2H), 4.92 (br t, J = 7.2 Hz, |
| | | | | 1H), 4.50 (br d, J = 9.2 Hz, 1H), 4.46-4.41 (m, 1H), 4.28 (br s, |
| | | | | 1H), 3.89 (br d, J = 13.2 Hz, 2H), 3.75 (s, 3H), 3.65 (s, 3H), 3.61- |
| | | | | 3.57 (m, 2H), 2.81 (br s, 2H), 2.45 (s, 3H), 2.44 (br s, 2H), 2.04- |
| | | | | 1.99 (m, 1H), 1.88 (s, 3H), 1.80 (br dd, J = 4.8, 8.4 Hz, 1H), 1.72 |
| | | | | (br s, 1H), 1.59 (br d, J = 0.8 Hz, 2H), 1.52 (br dd, J = 2.0, 15.2 Hz, |
| | | | | 2H), 1.38 (d, J = 7.2 Hz, 3H), 1.26 (br t, J = 6.8 Hz, 1H), 0.93 (s, |
| | | | | 9H), 0.89 (s, 9H) |
| I-457 | A | HG | 1247.5 | 12.03-11.66 (m, 1H), 8.98 (s, 1H), 8.38 (br d, J = 7.6 Hz, 2H), |
| | | | | 8.13 (s, 1H), 8.01-7.88 (m, 1H), 7.88-7.76 (m, 2H), 7.69-7.53 |
| | | | | (m, 3H), 7.46-7.41 (m, 3H), 7.40-7.36 (m, 2H), 7.34-7.15 (m, |
| | | | | 2H), 7.03-6.83 (m, 2H), 5.11 (d, J = 3.2 Hz, 1H), 4.97-4.87 (m, |
| | | | | 1H), 4.53-4.47 (m, 1H), 4.43 (br t, J = 8.0 Hz, 1H), 4.28 (br s, |
| | | | | 1H), 3.96-3.78 (m, 4H), 3.64 (s, 3H), 3.61-3.53 (m, 2H), 2.85- |
| | | | | 2.72 (m, 2H), 2.45 (s, 3H), 2.43 (br d, J = 4.4 Hz, 2H), 2.31-2.25 |
| | | | | (m, 1H), 2.05-1.94 (m, 2H), 1.89 (br s, 2H), 1.84-1.68 (m, 3H), |
| | | | | 1.61 (br d, J = 1.6 Hz, 7H), 1.53 (br d, J = 9.6 Hz, 3H), 1.38 (br d, |
| | | | | J = 7.2 Hz, 3H), 1.28-1.20 (m, 2H), 0.93 (s, 9H) |
| I-458^{m} | HH | HK | 1168.7 | 11.79-11.61 (m, 2H), 9.04 (s, 1H), 8.38 (br d, J = 8.0 Hz, 1H), |
| | | | | 8.31 (s, 1H), 8.06 (br d, J = 6.8 Hz, 1H), 7.98 (br d, J = 8.4 Hz, |
| | | | | 1H), 7.82 (br d, J = 9.2 Hz, 1H), 7.59 (br d, J = 7.2 Hz, 1H), 7.52 |
| | | | | (br d, J = 8.8 Hz, 1H), 7.46-7.41 (m, 3H), 7.40-7.36 (m, 3H), |
| | | | | 7.30 (s, 1H), 7.02 (br d, J = 8.8 Hz, 1H), 4.97 (br s, 2H), 4.94-4.88 |
| | | | | (m, 1H), 4.51 (br d, J = 9.2 Hz, 1H), 4.44-4.39 (m, 1H), 4.27 (br |
| | | | | s, 1H), 3.87 (br d, J = 7.2 Hz, 3H), 3.59 (br s, 2H), 3.31-3.25 (m, |
| | | | | 2H), 3.06-3.00 (m, 2H), 2.46 (s, 3H), 2.37 (s, 3H), 2.27-2.20 (m, |
| | | | | 1H), 2.10 (s, 3H), 2.00 (br d, J = 8.8 Hz, 1H), 1.83-1.75 (m, 2H), |
| | | | | 1.70-1.57 (m, 6H), 1.53 (br d, J = 13.2 Hz, 3H), 1.49-1.43 (m, |
| | | | | 2H), 1.37 (br d, J = 6.8 Hz, 3H), 1.34-1.22 (m, 3H), 1.20-1.08 |
| | | | | (m, 4H), 0.98 (br s, 1H), 0.92 (s, 9H) |
| I-459 | A | HL | 1229.6 | 9.02-8.93 (m, 1H), 8.41-8.33 (m, 1H), 7.89-7.77 (m, 2H), 7.68- |
| | | | | 7.60 (m, 1H), 7.52-7.26 (m, 11H), 6.94 (br d, J = 4.8 Hz, 1H), |
| | | | | 5.10 (d, J = 3.6 Hz, 1H), 4.97-4.88 (m, 3H), 4.51 (br d, J = 9.6 |
| | | | | Hz, 1H), 4.43 (t, J = 8.0 Hz, 1H), 4.27 (br s, 1H), 3.93 (br t, J = 5.6 |
| | | | | Hz, 2H), 3.84 (br d, J = 7.2 Hz, 2H), 3.60 (br s, 2H), 3.18 (br d, J = |
| | | | | 6.4 Hz, 2H), 3.02 (br t, J = 4.8 Hz, 2H), 2.45 (s, 3H), 2.23 (td, J = |
| | | | | 7.6, 14.4 Hz, 1H), 2.12 (s, 3H), 1.99 (br d, J = 10.0 Hz, 1H), 1.79 |
| | | | | (ddd, J = 4.4, 8.0, 12.4 Hz, 2H), 1.69-1.41 (m, 11H), 1.37 (br d, J = |
| | | | | 6.8 Hz, 3H), 1.31-1.23 (m, 3H), 1.19-1.11 (m, 3H), 0.93 (s, |
| | | | | 9H) |
| I-460 | A | HU | 1174.7 | 12.52-12.27 (m, 1H), 12.03-11.54 (m, 1H), 9.01-8.93 (m, 1H), |
| | | | | 8.41-8.35 (m, 1H), 8.16-8.13 (m, 1H), 7.83 (br d, J = 9.2 Hz, |
| | | | | 1H), 7.51 (br d, J = 7.6 Hz, 1H), 7.45-7.27 (m, 8H), 7.21 (d, J = |
| | | | | 1.2 Hz, 1H), 6.97-6.74 (m, 1H), 5.10 (br d, J = 3.6 Hz, 1H), 4.95- |
| | | | | 4.89 (m, 1H), 4.85 (br s, 2H), 4.51 (d, J = 9.6 Hz, 1H), 4.42 (br t, |
| | | | | J = 8.0 Hz, 1H), 4.27 (br s, 1H), 3.91 (br t, J = 5.6 Hz, 2H), 3.84 |
| | | | | (br d, J = 7.2 Hz, 2H), 3.60 (br s, 2H), 2.99 (br s, 2H), 2.45 (s, 3H), |
| | | | | 2.39 (br d, J = 1.2 Hz, 3H), 2.23 (br dd, J = 6.8, 14.4 Hz, 1H), 2.16- |
| | | | | 2.08 (m, 4H), 2.04-1.97 (m, 1H), 1.78 (ddd, J = 4.4, 8.4, 12.8 |
| | | | | Hz, 2H), 1.70-1.42 (m, 11H), 1.37 (br d, J = 7.2 Hz, 3H), 1.33- |
| | | | | 1.25 (m, 2H), 1.19-1.11 (m, 3H), 1.03-0.95 (m, 2H), 0.93 (s, 9H) |
| I-461 | HV | HK | 1158.9 | 11.71 (br s, 1H), 9.07-8.99 (m, 1H), 8.94 (t, J = 5.6 Hz, 1H), 8.37 |
| | | | | (br d, J = 7.6 Hz, 1H), 8.32 (s, 1H), 7.82 (br d, J = 9.2 Hz, 1H), |
| | | | | 7.54 (d, J = 8.8 Hz, 1H), 7.45-7.41 (m, 2H), 7.39-7.36 (m, 2H), |
| | | | | 7.34-7.27 (m, 4H), 7.10 (s, 1H), 6.87 (d, J = 8.8 Hz, 1H), 4.91 (br |
| | | | | t, J = 7.2 Hz, 1H), 4.81 (s, 2H), 4.56 (d, J = 5.6 Hz, 2H), 4.51 (br |
| | | | | d, J = 9.2 Hz, 1H), 4.42 (br t, J = 8.0 Hz, 1H), 4.27 (br s, 1H), 3.92- |
| | | | | 3.84 (m, 6H), 3.34-3.29 (m, 2H), 2.98 (br s, 2H), 2.46 (s, 3H), |
| | | | | 2.36-2.20 (m, 2H), 2.12 (s, 3H), 2.04-1.97 (m, 1H), 1.82-1.75 |
| | | | | (m, 2H), 1.71-1.58 (m, 6H), 1.57-1.44 (m, 5H), 1.38-1.31 (m, |
| | | | | 5H), 1.20-1.12 (m, 3H), 0.99 (br s, 1H), 0.92 (s, 9H) |
| I-462 | A | HW | 1174.8 | 12.69-12.32 (m, 1H), 11.97-11.51 (m, 1H), 8.98 (s, 1H), 8.37 (br |
| | | | | d, J = 7.2 Hz, 1H), 7.85-7.77 (m, 1H), 7.56-7.46 (m, 2H), 7.45- |
| | | | | 7.41 (m, 2H), 7.41-7.29 (m, 5H), 7.03-6.76 (m, 2H), 5.09 (br d, |
| | | | | J = 2.8 Hz, 1H), 4.97-4.82 (m, 3H), 4.54-4.48 (m, 1H), 4.43 (br |
| | | | | t, J = 7.6 Hz, 1H), 4.27 (br s, 1H), 3.91 (br s, 2H), 3.84 (br d, J = |
| | | | | 6.4 Hz, 2H), 3.60 (br s, 2H), 2.99 (br s, 2H), 2.45 (s, 3H), 2.30 (s, |
| | | | | 3H), 2.24 (br dd, J = 6.4, 14.4 Hz, 1H), 2.12 (br s, 4H), 2.04-1.97 |
| | | | | (m, 1H), 1.83-1.74 (m, 2H), 1.71-1.40 (m, 11H), 1.37 (br d, J = |
| | | | | 6.8 Hz, 3H), 1.31 (br s, 2H), 1.20-1.11 (m, 3H), 0.98 (br s, 2H), |
| | | | | 0.93 (br s, 9H) |
| I-463 | A | HY | 1243.4 | 12.85 (br s, 1H), 11.78 (br d, J = 3.2 Hz, 1H), 8.98 (s, 1H), 8.44- |
| | | | | 8.38 (m, 1H), 8.05-8.00 (m, 1H), 7.79 (br d, J = 8.0 Hz, 1H), 7.63 |
| | | | | (br d, J = 7.2 Hz, 1H), 7.52 (br d, J = 8.4 Hz, 1H), 7.50-7.46 (m, |
| | | | | 1H), 7.45-7.41 (m, 3H), 7.39-7.33 (m, 5H), 7.28 (s, 1H), 6.97 (s, |
| | | | | 1H), 5.12 (d, J = 3.6 Hz, 1H), 4.99-4.93 (m, 2H), 4.90 (br t, J = |
| | | | | 7.2 Hz, 1H), 4.53 (d, J = 9.6 Hz, 1H), 4.48-4.41 (m, 1H), 4.28 (br |
| | | | | s, 1H), 3.97-3.90 (m, 4H), 3.84 (br d, J = 7.2 Hz, 2H), 3.73 (br d, |
| | | | | J = 6.0 Hz, 2H), 3.62-3.52 (m, 8H), 3.05-2.99 (m, 2H), 2.45 (s, |
| | | | | 3H), 2.10 (s, 3H), 2.08-1.99 (m, 1H), 1.82-1.73 (m, 2H), 1.68- |
| | | | | 1.59 (m, 3H), 1.53 (br d, J = 12.0 Hz, 2H), 1.35 (d, J = 7.2 Hz, 3H), |
| | | | | 1.18-1.09 (m, 3H), 1.01-0.95 (m, 2H), 0.92 (s, 9H) |
| I-464 | HZ | FQ | 1153.2 | 11.21-10.67 (m, 1H), 8.48 (d, J = 7.2 Hz, 1H), 7.96-7.84 (m, |
| | | | | 1H), 7.82-7.73 (m, 3H), 7.67 (br s, 1H), 7.60-7.51 (m, 2H), 7.47 |
| | | | | (br d, J = 8.4 Hz, 2H), 7.38 (brt, J = 7.2 Hz, 1H), 7.21 (br t, J = 7.2 |
| | | | | Hz, 1H), 7.08 (br d, J = 8.4 Hz, 1H), 5.11 (d, J = 3.6 Hz, 1H), 4.92 |
| | | | | (br t, J = 7.2 Hz, 1H), 4.50 (br d, J = 9.2 Hz, 1H), 4.43 (t, J = 8.0 |
| | | | | Hz, 1H), 4.25 (br s, 1H), 3.72 (s, 2H), 3.66-3.55 (m, 5H), 2.95- |
| | | | | 2.83 (m, 2H), 2.35 (br s, 3H), 2.27-2.22 (m, 3H), 2.21-2.16 (m, |
| | | | | 1H), 2.15-2.07 (m, 1H), 2.04-1.97 (m, 1H), 1.94 (br s, 3H), 1.73 |
| | | | | (ddd, J = 4.8, 8.4, 12.8 Hz, 1H), 1.69-1.63 (m, 3H), 1.62-1.53 |
| | | | | (m, 10H), 1.51-1.41 (m, 4H), 1.34 (br d, J = 7.2 Hz, 3H), 1.28- |
| | | | | 1.18 (m, 3H), 0.92 (s, 9H) |
| I-465 | A | IA | 1184.8 | 12.91-12.77 (m, 1H), 11.85-11.65 (m, 1H), 8.98 (s, 1H), 8.37 (br |
| | | | | d, J = 7.6 Hz, 1H), 8.02 (d, J = 7.6 Hz, 1H), 7.79 (br dd, J = 4.4, |
| | | | | 8.6 Hz, 2H), 7.63 (br d, J = 7.6 Hz, 1H), 7.54-7.46 (m, 2H), 7.45- |
| | | | | 7.42 (m, 3H), 7.39-7.33 (m, 4H), 7.29 (br s, 1H), 7.05-6.89 (m, |
| | | | | 1H), 5.10 (d, J = 3.8 Hz, 1H), 4.98-4.88 (m, 3H), 4.51 (d, J = 9.2 |
| | | | | Hz, 1H), 4.47-4.40 (m, 1H), 4.27 (br d, J = 1.6 Hz, 1H), 3.94 (br |
| | | | | t, J = 5.8 Hz, 2H), 3.82 (s, 2H), 3.65-3.55 (m, 3H), 3.02 (br s, 2H), |
| | | | | 2.45 (s, 3H), 2.22 (br dd, J = 7.6, 14.3 Hz, 1H), 2.12-2.07 (m, 3H), |
| | | | | 2.04-1.98 (m, 1H), 1.82-1.75 (m, 1H), 1.64-1.56 (m, 2H), 1.48- |
| | | | | 1.42 (m, 2H), 1.37 (d, J = 7.6 Hz, 3H), 1.34-1.22 (m, 3H), 1.19- |
| | | | | 1.06 (m, 1H), 0.92 (s, 18H) |
| I-466^{b} | A | ID | 594.0 | 13.14-12.83 (m, 1H), 11.93-11.59 (m, 1H), 8.98 (s, 1H), 8.51 (br |
| | | | | d, J = 3.6 Hz, 1H), 8.37 (br d, J = 7.2 Hz, 1H), 8.15 (br d, J = 8.4 |
| | | | | Hz, 1H), 7.80 (br d, J = 8.8 Hz, 1H), 7.64 (br d, J = 7.6 Hz, 1H), |
| | | | | 7.52 (br dd, J = 4.8, 7.9 Hz, 2H), 7.46-7.41 (m, 3H), 7.40-7.34 |
| | | | | (m, 3H), 7.33-7.27 (m, 1H), 7.01 (br d, J = 2.0 Hz, 1H), 5.10 (br |
| | | | | s, 1H), 4.96 (br s, 2H), 4.93-4.86 (m, 1H), 4.54-4.48 (m, 1H), |
| | | | | 4.42 (br t, J = 8.0 Hz, 1H), 4.27 (br d, J = 1.2 Hz, 1H), 3.94 (br s, |
| | | | | 2H), 3.82 (s, 2H), 3.65-3.56 (m, 3H), 3.26 (br s, 2H), 3.03 (br s, |
| | | | | 2H), 2.45 (s, 3H), 2.26-2.19 (m, 1H), 2.11 (s, 3H), 2.03-1.96 (m, |
| | | | | 1H), 1.83-1.75 (m, 1H), 1.60 (br s, 2H), 1.45 (br dd, J = 6.4, 13.8 |
| | | | | Hz, 2H), 1.37 (br d, J = 6.8 Hz, 3H), 1.30 (br d, J = 6.4 Hz, 2H), |
| | | | | 0.92 (s, 18H) |
| I-467^{b} | EP | ID | 586.5 | 13.05-12.93 (m, 1H), 11.81-11.69 (m, 1H), 8.98 (s, 1H), 8.56 (br |
| | | | | t, J = 5.6 Hz, 1H), 8.51 (d, J = 4.8 Hz, 1H), 8.15 (d, J = 8.0 Hz, |
| | | | | 1H), 7.86 (br d, J = 9.2 Hz, 1H), 7.64 (br d, J = 7.2 Hz, 1H), 7.52 |
| | | | | (dd, J = 4.8, 8.0 Hz, 2H), 7.43 (br d, J = 5.2 Hz, 2H), 7.41-7.36 |
| | | | | (m, 4H), 7.29 (br s, 1H), 7.06-6.97 (m, 1H), 5.13 (br d, J = 2.8 |
| | | | | Hz, 1H), 4.96 (br s, 2H), 4.53 (d, J = 9.2 Hz, 1H), 4.46-4.39 (m, |
| | | | | 2H), 4.35 (br s, 1H), 4.21 (dd, J = 5.2, 15.6 Hz, 1H), 3.94 (br t, J = |
| | | | | 5.6 Hz, 2H), 3.82 (s, 2H), 3.69-3.61 (m, 2H), 3.26-3.22 (m, 2H), |
| | | | | 3.02 (br t, J = 5.2 Hz, 2H), 2.43 (s, 3H), 2.29-2.19 (m, 1H), 2.11 |
| | | | | (s, 3H), 2.06 (br d, J = 8.8 Hz, 1H), 2.04-1.99 (m, 1H), 1.90 (ddd, |
| | | | | J = 4.4, 8.4, 12.5 Hz, 1H), 1.67-1.55 (m, 2H), 1.52-1.39 (m, 2H), |
| | | | | 1.35-1.23 (m, 2H), 0.92 (s, 18H) |
| I-468 | EO | ID | 1099.1 | 12.99 (br s, 1H), 11.73 (br d, J = 0.8 Hz, 1H), 8.57-8.49 (m, 2H), |
| | | | | 8.15 (br d, J = 8.4 Hz, 1H), 7.85 (br d, J = 9.6 Hz, 1H), 7.64 (br d, |
| | | | | J = 7.6 Hz, 1H), 7.55-7.49 (m, 2H), 7.47-7.43 (m, 1H), 7.39 (br |
| | | | | d, J = 8.0 Hz, 1H), 7.37-7.34 (m, 2H), 7.33-7.30 (m, 2H), 7.28 |
| | | | | (s, 1H), 7.03 (br d, J = 9.2 Hz, 1H), 5.12 (d, J = 2.8 Hz, 1H), 4.97 |
| | | | | (s, 2H), 4.53 (d, J = 9.6 Hz, 1H), 4.44-4.36 (m, 2H), 4.33 (br s, |
| | | | | 1H), 4.16 (br dd, J = 5.2, 16.4 Hz, 1H), 4.12 (s, 1H), 3.97-3.90 |
| | | | | (m, 2H), 3.82 (s, 2H), 3.64 (br s, 2H), 3.28-3.23 (m, 3H), 3.05- |
| | | | | 3.00 (m, 2H), 2.29-2.19 (m, 1H), 2.11 (s, 3H), 1.99 (br d, J = 8.8 |
| | | | | Hz, 1H), 1.92-1.82 (m, 1H), 1.66-1.56 (m, 2H), 1.50-1.39 (m, |
| | | | | 2H), 1.34-1.26 (m, 2H), 0.92 (s, 18H) |
| I-469 | FH | ID | 1112.3 | 13.08-12.92 (m, 1H), 11.85-11.63 (m, 1H), 8.51 (dd, J = 1.2, 4.8 |
| | | | | Hz, 1H), 8.35 (br d, J = 8.0 Hz, 1H), 8.18-8.12 (m, 1H), 7.79 (br |
| | | | | d, J = 9.6 Hz, 1H), 7.64 (br d, J = 7.6 Hz, 1H), 7.52 (dd, J = 4.8, |
| | | | | 8.0 Hz, 2H), 7.47-7.43 (m, 1H), 7.42 (s, 1H), 7.40 (s, 1H), 7.38- |
| | | | | 7.35 (m, 1H), 7.29 (s, 2H), 7.27 (s, 1H), 7.06-6.95 (m, 1H), 5.09 |
| | | | | (br d, J = 2.8 Hz, 1H), 4.96 (br s, 2H), 4.87 (quin, J = 6.8 Hz, 1H), |
| | | | | 4.50 (d, J = 9.2 Hz, 1H), 4.40 (t, J = 8.0 Hz, 1H), 4.26 (br s, 1H), |
| | | | | 4.12 (s, 1H), 3.94 (br t, J = 5.6 Hz, 2H), 3.82 (s, 2H), 3.62-3.55 |
| | | | | (m, 2H), 3.25 (br d, J = 6.4 Hz, 1H), 3.03 (br t, J = 5.2 Hz, 2H), |
| | | | | 2.26-2.17 (m, 1H), 2.11 (s, 3H), 2.09-2.03 (m, 1H), 2.02-1.94 |
| | | | | (m, 1H), 1.78-1.70 (m, 1H), 1.60 (br d, J = 3.6 Hz, 2H), 1.52- |
| | | | | 1.37 (m, 3H), 1.32 (br d, J = 7.2 Hz, 3H), 1.30-1.23 (m, 2H), 0.92 |
| | | | | (d, J = 2.8 Hz, 18H) |
| I-470 | FH | IA | 1112.1 | 12.98-12.68 (m, 1H), 12.01-11.46 (m, 1H), 8.41-8.33 (m, 1H), |
| | | | | 8.03 (d, J = 7.6 Hz, 1H), 7.79 (br d, J = 8.0 Hz, 2H), 7.62 (br d, J = |
| | | | | 8.0 Hz, 1H), 7.51-7.31 (m, 8H), 7.28 (d, J = 8.4 Hz, 2H), 5.09 |
| | | | | (d, J = 3.6 Hz, 1H), 4.99-4.79 (m, 3H), 4.50 (d, J = 9.6 Hz, 1H), |
| | | | | 4.40 (t, J = 7.6 Hz, 1H), 4.25 (br s, 1H), 4.12 (s, 1H), 3.93 (br t, J = |
| | | | | 5.6 Hz, 2H), 3.81 (s, 2H), 3.64-3.55 (m, 2H), 3.02 (br d, J = 4.0 |
| | | | | Hz, 2H), 2.21 (br dd, J = 7.2, 14.3 Hz, 1H), 2.15-2.06 (m, 4H), |
| | | | | 2.02-1.93 (m, 1H), 1.78-1.70 (m, 1H), 1.63-1.50 (m, 2H), 1.44 |
| | | | | (br dd, J = 7.2, 14.3 Hz, 2H), 1.32 (d, J = 7.2 Hz, 3H), 1.27-1.19 |
| | | | | (m, 4H), 0.92 (s, 18H) |
| I-471 | EO | IA | 1097.9 | 13.05-12.59 (m, 1H), 12.28-11.30 (m, 1H), 8.58 (s, 1H), 8.03 (d, |
| | | | | J = 8.0 Hz, 1H), 7.85 (br d, J = 9.2 Hz, 1H), 7.79 (d, J = 8.0 Hz, |
| | | | | 1H), 7.62 (br d, J = 7.6 Hz, 1H), 7.50-7.40 (m, 3H), 7.38-7.31 |
| | | | | (m, 7H), 6.97-6.74 (m, 1H), 5.12 (br s, 1H), 4.91 (br s, 2H), 4.53 |
| | | | | (d, J = 9.6 Hz, 1H), 4.43-4.36 (m, 2H), 4.33 (br s, 1H), 4.21-4.09 |
| | | | | (m, 2H), 3.93 (br t, J = 5.6 Hz, 2H), 3.81 (s, 2H), 3.68-3.61 (m, |
| | | | | 2H), 3.01 (br d, J = 5.2 Hz, 3H), 2.22 (br dd, J = 7.2, 14.4 Hz, 1H), |
| | | | | 2.15-1.98 (m, 6H), 1.92-1.83 (m, 1H), 1.60-1.50 (m, 2H), 1.48- |
| | | | | 1.40 (m, 2H), 1.30-1.22 (m, 2H), 0.92 (s, 18H) |
| I-472 | EO | IE | 1059.5 | 11.93-11.53 (m, 1H), 8.53 (br t, J = 5.9 Hz, 1H), 8.13 (s, 1H), 7.95- |
| | | | | 7.75 (m, 3H), 7.70 (d, J = 8.6 Hz, 1H), 7.65-7.50 (m, 1H), 7.43- |
| | | | | 7.28 (m, 7H), 7.26-7.16 (m, 1H), 4.52 (d, J = 9.3 Hz, 1H), 4.45- |
| | | | | 4.36 (m, 2H), 4.33 (br s, 1H), 4.12 (s, 1H), 4.16 (br dd, J = 5.4, |
| | | | | 16.1 Hz, 1H), 3.87 (s, 2H), 3.72-3.58 (m, 6H), 3.33 (br d, J = 7.5 |
| | | | | Hz, 2H), 2.39 (s, 3H), 2.26-2.17 (m, 4H), 2.12-1.96 (m, 2H), |
| | | | | 1.86 (ddd, J = 4.6, 8.4, 12.6 Hz, 1H), 1.69-1.56 (m, 2H), 1.51- |
| | | | | 1.38 (m, 2H), 1.36-1.25 (m, 2H), 0.95 (s, 9H), 0.91 (s, 9H) |
| I-473 | FH | IE | 1073.5 | 11.95-11.66 (m, 1H), 11.30-10.84 (m, 1H), 8.35 (d, J = 7.6 Hz, |
| | | | | 1H), 8.14 (s, 1H), 7.88 (br s, 1H), 7.84-7.75 (m, 2H), 7.69 (br d, |
| | | | | J = 8.4 Hz, 1H), 7.64-7.50 (m, 1H), 7.44-7.37 (m, 4H), 7.34 (br |
| | | | | d, J = 8.0 Hz, 1H), 7.31-7.26 (m, 2H), 7.25-7.17 (m, 1H), 5.09 |
| | | | | (d, J = 3.2 Hz, 1H), 4.87 (quin, J = 7.2 Hz, 1H), 4.54-4.47 (m, |
| | | | | 1H), 4.40 (t, J = 8.0 Hz, 1H), 4.31-4.24 (m, 1H), 4.13 (s, 1H), 3.87 |
| | | | | (s, 2H), 3.66 (s, 3H), 3.58 (br s, 2H), 3.47-3.36 (m, 1H), 2.46- |
| | | | | 2.36 (m, 3H), 2.25-2.16 (m, 4H), 2.12-2.03 (m, 1H), 2.02-1.94 |
| | | | | (m, 1H), 1.81-1.71 (m, 1H), 1.69-1.56 (m, 2H), 1.49-1.39 (m, |
| | | | | 2H), 1.39-1.26 (m, 5H), 0.96 (s, 9H), 0.91 (s, 9H) |
| I-474ⁿ | IF | FQ | 1168.6 | 8.40-8.31 (m, 1H), 7.93-7.54 (m, 5H), 7.47-7.34 (m, 4H), 7.33- |
| | | | | 7.17 (m, 4H), 5.10 (d, J = 3.6 Hz, 1H), 4.86-4.79 (m, 1H), 4.76- |
| | | | | 4.70 (m, 1H), 4.53-4.41 (m, 2H), 4.29-4.23 (m, 1H), 4.16-4.10 |
| | | | | (m, 1H), 3.73 (s, 2H), 3.62 (br d, J = 9.2 Hz, 5H), 3.58-3.49 (m, |
| | | | | 3H), 2.41-2.33 (m, 3H), 2.25-2.17 (m, 4H), 2.12-2.06 (m, 1H), |
| | | | | 2.00-1.91 (m, 4H), 1.81-1.74 (m, 1H), 1.68-1.51 (m, 15H), 1.48- |
| | | | | 1.39 (m, 2H), 1.28 (br d, J = 6.4 Hz, 2H), 0.91 (s, 9H) |
| I-475 | IG | IH | 1167.5 | 9.78 (s, 1H), 8.25 (br d, J = 6.4 Hz, 1H), 7.99-7.82 (m, 1H), 7.77 |
| | | | | (br d, J = 9.2 Hz, 2H), 7.69-7.53 (m, 2H), 7.42-7.32 (m, 2H), |
| | | | | 7.24-7.17 (m, 1H), 7.12 (d, J = 8.0 Hz, 1H), 6.87-6.82 (m, 2H), |
| | | | | 5.09 (br d, J = 4.0 Hz, 2H), 4.49 (br d, J = 9.2 Hz, 1H), 4.43 (s, |
| | | | | 1H), 4.26 (br s, 1H), 4.03 (s, 1H), 3.73 (br s, 2H), 3.66-3.55 (m, |
| | | | | 6H), 2.37 (br s, 3H), 2.20 (br s, 4H), 2.07 (s, 1H), 2.01-1.91 (m, |
| | | | | 5H), 1.79-1.74 (m, 1H), 1.64 (br s, 4H), 1.58 (br s, 3H), 1.55 (br |
| | | | | s, 7H), 1.47-1.41 (m, 2H), 1.25 (br d, J = 6.8 Hz, 6H), 0.91 (s, |
| | | | | 9H) |
| I-476 | II | IH | 1181.5 | 12.07-11.57 (m, 1H), 11.41-10.64 (m, 1H), 8.40 (d, J = 8.0 Hz, |
| | | | | 1H), 8.13 (s, 1H), 7.95-7.85 (m, 1H), 7.80 (br d, J = 9.6 Hz, 2H), |
| | | | | 7.72-7.64 (m, 1H), 7.62-7.53 (m, 1H), 7.46-7.28 (m, 7H), 7.25- |
| | | | | 7.18 (m, 1H), 5.08 (d, J = 3.6 Hz, 1H), 4.99-4.91 (m, 1H), 4.54- |
| | | | | 4.42 (m, 2H), 4.24 (br d, J = 2.0 Hz, 1H), 4.13 (s, 1H), 3.75 (s, |
| | | | | 2H), 3.65 (s, 3H), 3.58 (br s, 2H), 3.53 (br dd, J = 6.6, 9.8 Hz, 1H), |
| | | | | 3.46-3.42 (m, 1H), 3.22 (s, 3H), 2.39 (br s, 3H), 2.26-2.15 (m, |
| | | | | 4H), 2.12-1.89 (m, 6H), 1.78-1.71 (m, 1H), 1.70-1.51 (m, 15H), |
| | | | | 1.48-1.39 (m, 2H), 1.36-1.24 (m, 2H), 1.00-0.86 (m, 9H) |
| I-477 | IJ | IH | 1212.2 | 11.90-11.67 (m, 1H), 11.12-10.95 (m, 1H), 8.25 (d, J = 8.0 Hz, |
| | | | | 1H), 7.98-7.84 (m, 1H), 7.77 (br d, J = 9.2 Hz, 2H), 7.69 (br d, J = |
| | | | | 8.8 Hz, 1H), 7.42-7.31 (m, 3H), 7.25-7.16 (m, 2H), 7.03-6.99 |
| | | | | (m, 2H), 5.18-5.05 (m, 2H), 4.88 (br d, J = 4.8 Hz, 1H), 4.49 (d, |
| | | | | J = 9.6 Hz, 1H), 4.41 (t, J = 8.0 Hz, 1H), 4.26 (br s, 1H), 4.12 (s, |
| | | | | 1H), 4.06-3.98 (m, 2H), 3.76-3.71 (m, 4H), 3.65 (s, 3H), 3.57 |
| | | | | (br s, 2H), 2.39 (br s, 3H), 2.18 (s, 3H), 2.11-2.04 (m, 1H), 2.03- |
| | | | | 1.97 (m, 1H), 1.94 (br s, 3H), 1.76 (dt, J = 4.4, 8.4 Hz, 1H), 1.68- |
| | | | | 1.62 (m, 5H), 1.58 (br s, 3H), 1.55 (br s, 7H), 1.49-1.41 (m, 2H), |
| | | | | 1.34-1.23 (m, 7H), 0.90 (s, 9H) |
| I-478 | II | IL | 1168.5 | 13.04-12.92 (m, 1H), 11.79-11.66 (m, 1H), 8.51 (dd, J = 1.2, 4.7 |
| | | | | Hz, 1H), 8.41 (br d, J = 8.0 Hz, 1H), 8.18-8.12 (m, 1H), 7.82 (br |
| | | | | d, J = 9.2 Hz, 1H), 7.63 (br d, J = 7.6 Hz, 1H), 7.52 (br dd, J = 4.8, |
| | | | | 8.2 Hz, 2H), 7.46-7.37 (m, 4H), 7.33-7.25 (m, 3H), 7.06-6.95 |
| | | | | (m, 1H), 5.08 (br d, J = 2.8 Hz, 1H), 4.95 (br s, 3H), 4.54-4.44 |
| | | | | (m, 2H), 4.24 (br s, 1H), 4.13 (s, 1H), 3.93 (br t, J = 6.0 Hz, 2H), |
| | | | | 3.84 (br d, J = 7.2 Hz, 2H), 3.58 (br s, 2H), 3.53 (dd, J = 6.4, 9.9 |
| | | | | Hz, 1H), 3.45 (dd, J = 6.0, 9.9 Hz, 1H), 3.22 (s, 3H), 3.02 (br s, |
| | | | | 2H), 2.27-2.19 (m, 1H), 2.11 (s, 3H), 2.02-1.95 (m, 1H), 1.80- |
| | | | | 1.70 (m, 2H), 1.70-1.47 (m, 9H), 1.47-1.39 (m, 2H), 1.35-1.22 |
| | | | | (m, 3H), 1.19-1.11 (m, 3H), 0.97 (br s, 2H), 0.92 (s, 9H) |
| I-479 | IG | IL | 1154.6 | 12.98 (br s, 1H), 11.73 (br d, J = 1.2 Hz, 1H), 9.78 (s, 1H), 8.51 |
| | | | | (dd, J = 1.6, 4.8 Hz, 1H), 8.24 (br d, J = 8.0 Hz, 1H), 8.15 (d, J = |
| | | | | 7.6 Hz, 1H), 7.79 (d, J = 9.2 Hz, 1H), 7.63 (br d, J = 7.2 Hz, 1H), |
| | | | | 7.52 (dd, J = 4.8, 8.0 Hz, 2H), 7.46-7.43 (m, 1H), 7.40-7.35 (m, |
| | | | | 1H), 7.26 (br s, 1H), 7.13 (d, J = 8.0 Hz, 1H), 6.89-6.82 (m, 2H), |
| | | | | 5.12-5.04 (m, 2H), 4.96 (br s, 2H), 4.50 (d, J = 9.2 Hz, 1H), 4.43 |
| | | | | (s, 1H), 4.26 (br d, J = 2.4 Hz, 1H), 4.03 (s, 1H), 3.93 (br t, J = 5.7 |
| | | | | Hz, 2H), 3.84 (br d, J = 7.4 Hz, 2H), 3.58 (br s, 2H), 3.03 (br d, J = |
| | | | | 4.4 Hz, 2H), 2.22 (br d, J = 8.0 Hz, 1H), 2.11 (s, 3H), 1.99 (br d, |
| | | | | J = 10.4 Hz, 2H), 1.81-1.74 (m, 2H), 1.66-1.58 (m, 4H), 1.55- |
| | | | | 1.51 (m, 2H), 1.45 (br d, J = 6.8 Hz, 2H), 1.35-1.22 (m, 9H), 1.14 |
| | | | | (br d, J = 8.0 Hz, 3H), 0.97 (br s, 2H), 0.92 (s, 9H) |
| I-480 | IJ | IL | 1200.4 | 12.98 (br s, 1H), 11.72 (br s, 1H), 8.51 (d, J = 4.0 Hz, 1H), 8.25 (br |
| | | | | d, J = 8.0 Hz, 1H), 8.15 (br d, J = 8.4 Hz, 1H), 7.79 (br d, J = 9.2 |
| | | | | Hz, 1H), 7.64 (br d, J = 7.2 Hz, 1H), 7.52 (dd, J = 4.4, 8.0 Hz, 2H), |
| | | | | 7.47-7.43 (m, 1H), 7.41-7.35 (m, 1H), 7.26 (s, 1H), 7.21-7.17 |
| | | | | (m, 1H), 7.05-6.99 (m, 3H), 5.18-5.12 (m, 1H), 5.08 (d, J = 3.6 |
| | | | | Hz, 1H), 4.96 (br s, 2H), 4.88 (t, J = 5.6 Hz, 1H), 4.51 (br d, J = |
| | | | | 9.6 Hz, 1H), 4.42 (br t, J = 8.0 Hz, 1H), 4.34 (t, J = 5.2 Hz, 1H), |
| | | | | 4.29-4.24 (m, 1H), 4.12 (s, 1H), 4.07-3.98 (m, 2H), 3.94 (br t, J = |
| | | | | 5.6 Hz, 2H), 3.85 (br d, J = 7.2 Hz, 2H), 3.73 (q, J = 4.8 Hz, 2H), |
| | | | | 3.59 (br s, 2H), 3.47-3.41 (m, 2H), 3.06-3.00 (m, 2H), 2.27- |
| | | | | 2.19 (m, 1H), 2.11 (s, 3H), 2.09-2.05 (m, 1H), 2.02-1.95 (m, |
| | | | | 1H), 1.78 (br dd, J = 4.4, 8.8 Hz, 2H), 1.62 (br d, J = 14.0 Hz, 5H), |
| | | | | 1.53 (br d, J = 11.6 Hz, 2H), 1.49-1.40 (m, 2H), 1.32 (br s, 1H), |
| | | | | 1.28 (br d, J = 6.8 Hz, 3H), 1.18-1.11 (m, 3H), 1.05 (t, J = 7.2 Hz, |
| | | | | 2H), 0.91 (s, 9H) |
| I-481 | IM | IH | 1164.1 | 11.86-11.70 (m, 1H), 11.12-10.95 (m, 1H), 8.79-8.71 (m, 1H), |
| | | | | 7.96-7.73 (m, 3H), 7.71-7.66 (m, 1H), 7.41-7.31 (m, 3H), 7.28- |
| | | | | 7.18 (m, 5H), 5.14-5.09 (m, 1H), 4.52 (d, J = 9.6 Hz, 1H), 4.38- |
| | | | | 4.30 (m, 2H), 4.08 (s, 1H), 3.75 (s, 2H), 3.68-3.58 (m, 5H), 2.41- |
| | | | | 2.35 (m, 3H), 2.28-2.14 (m, 5H), 2.11-2.06 (m, 1H), 1.97-1.91 |
| | | | | (m, 4H), 1.87-1.81 (m, 1H), 1.68-1.63 (m, 4H), 1.58 (br s, 3H), |
| | | | | 1.54 (br s, 7H), 1.48-1.41 (m, 2H), 1.34-1.28 (m, 2H), 1.21- |
| | | | | 1.14 (m, 3H), 1.12-1.07 (m, 1H), 0.90 (s, 8H) |
| I-482 | IM | IL | 1150.7 | 13.16-12.71 (m, 1H), 12.00-11.46 (m, 1H), 8.76 (s, 1H), 8.52 |
| | | | | (dd, J = 1.2, 4.6 Hz, 1H), 8.18-8.12 (m, 1H), 7.91 (br d, J = 9.2 |
| | | | | Hz, 1H), 7.64 (d, J = 7.6 Hz, 1H), 7.52 (br dd, J = 4.8, 8.2 Hz, 2H), |
| | | | | 7.45 (br d, J = 7.2 Hz, 1H), 7.40-7.36 (m, 1H), 7.30-7.24 (m, |
| | | | | 3H), 7.23-7.19 (m, 2H), 7.05-6.92 (m, 1H), 5.12 (br d, J = 2.0 |
| | | | | Hz, 1H), 4.95 (br s, 2H), 4.53 (d, J = 9.6 Hz, 1H), 4.35 (br d, J = |
| | | | | 8.0 Hz, 2H), 4.09 (s, 1H), 3.93 (br t, J = 6.0 Hz, 2H), 3.85 (br d, J = |
| | | | | 7.2 Hz, 2H), 3.63 (br s, 2H), 3.02 (br t, J = 5.6 Hz, 2H), 2.24 (br |
| | | | | dd, J = 7.2, 14.6 Hz, 1H), 2.11 (s, 3H), 1.97 (br dd, J = 7.6, 10.9 |
| | | | | Hz, 1H), 1.87-1.76 (m, 2H), 1.71-1.50 (m, 9H), 1.46 (br dd, J = |
| | | | | 7.2, 15.7 Hz, 2H), 1.33-1.27 (m, 2H), 1.23-1.10 (m, 8H), 1.04- |
| | | | | 0.96 (m, 2H), 0.92 (s, 9H) |
| I-483 | IN | IL | 1184.7 | 13.07-12.88 (m, 1H), 11.81-11.64 (m, 1H), 8.51 (d, J = 4.0 Hz, |
| | | | | 1H), 8.42 (br t, J = 6.0 Hz, 1H), 8.15 (d, J = 8.4 Hz, 1H), 7.80 (br |
| | | | | d, J = 9.6 Hz, 1H), 7.64 (br d, J = 7.6 Hz, 1H), 7.52 (dd, J = 4.8, |
| | | | | 8.1 Hz, 2H), 7.47-7.42 (m, 2H), 7.40-7.34 (m, 2H), 7.33-7.25 |
| | | | | (m, 2H), 7.05-6.97 (m, 1H), 5.09 (br d, J = 3.2 Hz, 1H), 4.96 (br |
| | | | | s, 2H), 4.53 (d, J = 9.6 Hz, 1H), 4.42-4.35 (m, 2H), 4.33-4.23 |
| | | | | (m, 2H), 3.99-3.90 (m, 5H), 3.86-3.79 (m, 5H), 3.69-3.58 (m, |
| | | | | 2H), 3.02 (br t, J = 5.6 Hz, 2H), 2.28-2.18 (m, 2H), 2.13-2.06 |
| | | | | (m, 4H), 2.05-1.92 (m, 2H), 1.90-1.76 (m, 2H), 1.69-1.57 (m, |
| | | | | 5H), 1.53 (br d, J = 12.0 Hz, 3H), 1.45 (br dd, J = 7.6, 15.4 Hz, |
| | | | | 2H), 1.36-1.22 (m, 3H), 1.14 (br d, J = 8.4 Hz, 3H), 0.93 (s, 9H) |
| I-484 | IO | IH | 1182.2 | 11.78 (br s, 1H), 11.23-10.86 (m, 1H), 8.53 (br t, J = 6.0 Hz, 1H), |
| | | | | 8.13 (s, 1H), 8.01-7.85 (m, 1H), 7.85-7.71 (m, 3H), 7.70-7.66 |
| | | | | (m, 1H), 7.56 (d, J = 8.4 Hz, 1H), 7.49-7.46 (m, 1H), 7.41-7.31 |
| | | | | (m, 3H), 7.26-7.18 (m, 1H), 7.01 (d, J = 8.4 Hz, 1H), 5.10 (d, J = |
| | | | | 3.2 Hz, 1H), 4.56-4.49 (m, 2H), 4.43 (br t, J = 8.0 Hz, 1H), 4.34 |
| | | | | (br s, 1H), 4.27 (br dd, J = 5.6, 15.6 Hz, 1H), 3.92 (s, 3H), 3.75 (s, |
| | | | | 2H), 3.69-3.62 (m, 5H), 2.45-2.42 (m, 4H), 2.39 (br s, 2H), 2.22- |
| | | | | 2.16 (m, 4H), 2.13-1.99 (m, 3H), 1.94 (br s, 4H), 1.68-1.62 (m, |
| | | | | 5H), 1.58 (br s, 3H), 1.55 (br s, 7H), 1.45 (br dd, J = 6.4, 14.0 Hz, |
| | | | | 2H), 1.35-1.28 (m, 2H), 0.95-0.89 (m, 9H) |
| I-485^{o} | IV | IK | 1155.5 | 12.98 (br s, 1H), 11.74 (br s, 1H), 10.31 (s, 1H), 8.52 (d, J = 3.2 |
| | | | | Hz, 1H), 8.45 (br d, J = 7.2 Hz, 1H), 8.15 (br d, J = 8.0 Hz, 1H), |
| | | | | 7.84 (br d, J = 8.8 Hz, 1H), 7.64 (br d, J = 7.2 Hz, 1H), 7.59 (d, J = |
| | | | | 8.0 Hz, 1H), 7.52 (dd, J = 4.8, 8.0 Hz, 2H), 7.45 (br d, J = 7.6 |
| | | | | Hz, 1H), 7.39 (br d, J = 7.6 Hz, 1H), 7.26 (br s, 1H), 7.11-6.96 |
| | | | | (m, 3H), 5.10 (br d, J = 3.2 Hz, 1H), 4.96 (br s, 3H), 4.49 (br d, J = |
| | | | | 9.6 Hz, 2H), 4.33 (br s, 1H), 3.98-3.89 (m, 2H), 3.85 (br d, J = |
| | | | | 7.2 Hz, 2H), 3.61 (br s, 2H), 3.28-3.25 (m, 2H), 3.06-3.00 (m, |
| | | | | 2H), 2.26-2.21 (m, 1H), 2.11 (s, 4H), 2.02-1.97 (m, 1H), 1.89 |
| | | | | (br dd, J = 4.8, 8.4 Hz, 1H), 1.81-1.75 (m, 1H), 1.67-1.59 (m, |
| | | | | 4H), 1.53 (br d, J = 12.0 Hz, 2H), 1.46 (br dd, J = 6.8, 14.8 Hz, |
| | | | | 2H), 1.28 (br d, J = 6.8 Hz, 5H), 1.14 (br d, J = 8.0 Hz, 3H), 1.01- |
| | | | | 0.90 (m, 3H), 0.83 (s, 9H) |
| I-486^{o} | IV | IK | 1155.5 | 12.98 (br d, J = 2.4 Hz, 1H), 11.74 (br s, 1H), 10.35 (s, 1H), 8.52 |
| | | | | (d, J = 4.8 Hz, 1H), 8.36 (br d, J = 7.2 Hz, 1H), 8.15 (br d, J = 8.0 |
| | | | | Hz, 1H), 7.79 (br d, J = 9.2 Hz, 1H), 7.64 (br d, J = 7.6 Hz, 1H), |
| | | | | 7.52 (dd, J = 4.8, 8.0 Hz, 2H), 7.47-7.43 (m, 1H), 7.39 (br d, J = |
| | | | | 7.6 Hz, 1H), 7.32-7.25 (m, 2H), 7.24-7.19 (m, 1H), 7.08 (s, 1H), |
| | | | | 7.05-6.97 (m, 1H), 5.10 (br s, 2H), 4.96 (br s, 2H), 4.52-4.39 (m, |
| | | | | 2H), 4.26 (br s, 1H), 3.93 (br t, J = 5.2 Hz, 2H), 3.85 (br d, J = 7.2 |
| | | | | Hz, 2H), 3.59 (br s, 2H), 3.28-3.22 (m, 2H), 3.03 (br s, 2H), 2.25- |
| | | | | 2.19 (m, 1H), 2.14-2.07 (m, 4H), 2.04-1.98 (m, 1H), 1.81-1.72 |
| | | | | (m, 2H), 1.67-1.59 (m, 4H), 1.56-1.51 (m, 2H), 1.46 (br d, J = |
| | | | | 6.8 Hz, 2H), 1.27 (br d, J = 6.8 Hz, 5H), 1.14 (br d, J = 8.0 Hz, |
| | | | | 3H), 1.03-0.85 (m, 12H) |
| I-487 | IW | IH | 1212.3 | 12.03-11.57 (m, 1H), 11.30-10.69 (m, 1H), 8.33-8.23 (m, 1H), |
| | | | | 7.94-7.82 (m, 1H), 7.78 (br d, J = 9.4 Hz, 2H), 7.71-7.64 (m, |
| | | | | 1H), 7.55-7.49 (m, 1H), 7.46-7.30 (m, 6H), 7.24-7.18 (m, 1H), |
| | | | | 5.12-5.03 (m, 1H), 5.01-4.91 (m, 1H), 4.49 (br d, J = 9.2 Hz, |
| | | | | 1H), 4.45-4.29 (m, 2H), 4.28-4.19 (m, 1H), 3.97 (d, J = 4.8 Hz, |
| | | | | 3H), 3.81 (d, J = 3.5 Hz, 3H), 3.74 (br s, 2H), 3.64 (br s, 3H), 3.58 |
| | | | | (br d, J = 4.4 Hz, 2H), 2.38 (br s, 3H), 2.19 (br s, 5H), 2.11-2.00 |
| | | | | (m, 2H), 1.93 (br s, 4H), 1.69-1.58 (m, 8H), 1.54 (br s, 7H), 1.48- |
| | | | | 1.42 (m, 2H), 1.36 (br d, J = 6.8 Hz, 3H), 1.32-1.22 (m, 3H), |
| | | | | 1.15 (br t, J = 7.6 Hz, 1H), 1.05 (t, J = 6.8 Hz, 1H), 0.92-0.86 (m, |
| | | | | 9H) |
| I-488^{b, n} | IF | IK | 578.0 | 13.28-12.71 (m, 1H), 12.08-11.42 (m, 1H), 8.51 (dd, J = 1.2, 4.8 |
| | | | | Hz, 1H), 8.34 (d, J = 8.0 Hz, 1H), 8.15 (dd, J = 1.2, 8.4 Hz, 1H), |
| | | | | 7.81 (d, J = 9.2 Hz, 1H), 7.64 (d, J = 7.6 Hz, 1H), 7.54-7.47 (m, |
| | | | | 2H), 7.46-7.36 (m, 4H), 7.33-7.25 (m, 3H), 6.97 (br s, 1H), 5.10 |
| | | | | (br s, 1H), 4.95 (s, 2H), 4.86-4.79 (m, 1H), 4.74 (t, J = 5.6 Hz, |
| | | | | 1H), 4.51 (d, J = 9.4 Hz, 1H), 4.45 (t, J = 8.0 Hz, 1H), 4.27 (br s, |
| | | | | 1H), 4.12 (s, 1H), 3.93 (brt, J = 5.6 Hz, 2H), 3.84 (br d, J = 7.6 Hz, |
| | | | | 2H), 3.62-3.50 (m, 4H), 3.24-3.16 (m, 2H), 3.02 (br t, J = 5.6 |
| | | | | Hz, 2H), 2.28-2.18 (m, 1H), 2.12-2.06 (m, 4H), 2.03-1.94 (m, |
| | | | | 1H), 1.78 (ddd, J = 4.4, 8.4, 12.6 Hz, 2H), 1.69-1.50 (m, 8H), 1.45 |
| | | | | (br dd, J = 7.2, 14.8 Hz, 2H), 1.29 (quin, J = 7.6 Hz, 2H), 1.17- |
| | | | | 1.09 (m, 3H), 0.92 (s, 10H) |
| I-489^{b, p} | IW | IL | 600.1 | 8.51 (d, J = 4.4 Hz, 1H), 8.31 (br d, J = 7.6 Hz, 1H), 8.14 (d, J = |
| | | | | 8.4 Hz, 1H), 7.79 (br d, J = 9.2 Hz, 1H), 7.62 (br d, J = 7.6 Hz, |
| | | | | 1H), 7.51 (dd, J = 4.4, 8.0 Hz, 1H), 7.47-7.28 (m, 7H), 7.00-6.63 |
| | | | | (m, 1H), 5.14-5.00 (m, 1H), 4.99-4.94 (m, 1H), 4.89 (br d, J = |
| | | | | 2.0 Hz, 1H), 4.50 (d, J = 9.6 Hz, 1H), 4.41 (br t, J = 7.6 Hz, 1H), |
| | | | | 4.24 (br s, 1H), 3.97 (s, 3H), 3.92 (brt, J = 5.2 Hz, 2H), 3.85-3.77 |
| | | | | (m, 5H), 3.59 (br s, 2H), 3.00 (br s, 2H), 2.23-2.17 (m, 1H), 2.14 |
| | | | | (s, 3H), 2.11-2.06 (m, 1H), 2.02-1.86 (m, 2H), 1.80-1.69 (m, |
| | | | | 2H), 1.68-1.60 (m, 3H), 1.56-1.50 (m, 3H), 1.48-1.40 (m, 3H), |
| | | | | 1.36 (br d, J = 6.8 Hz, 3H), 1.23 (br s, 3H), 1.18-1.13 (m, 3H), |
| | | | | 0.97 (br s, 2H), 0.93 (s, 9H), 0.87-0.83 (m, 1H) |
| I-490^{b, p} | IW | IL | 600.1 | 8.52 (d, J = 4.8 Hz, 1H), 8.27 (br d, J = 7.6 Hz, 1H), 8.15 (d, J = |
| | | | | 8.0 Hz, 1H), 7.80 (br d, J = 9.6 Hz, 1H), 7.63 (br d, J = 8.0 Hz, |
| | | | | 1H), 7.54-7.50 (m, 2H), 7.43 (br dd, J = 5.2, 7.2 Hz, 3H), 7.40- |
| | | | | 7.31 (m, 3H), 7.11-6.79 (m, 1H), 5.10 (br d, J = 4.4 Hz, 1H), 5.01- |
| | | | | 4.96 (m, 1H), 4.92 (br d, J = 0.8 Hz, 1H), 4.51 (br d, J = 9.2 Hz, |
| | | | | 1H), 4.42 (br t, J = 7.6 Hz, 1H), 4.34 (br s, 1H), 3.97 (s, 3H), 3.95- |
| | | | | 3.91 (m, 2H), 3.81 (s, 5H), 3.70-3.58 (m, 3H), 3.01 (br s, 2H), |
| | | | | 2.21 (br d, J = 6.0 Hz, 1H), 2.14 (br s, 3H), 2.11-2.06 (m, 1H), |
| | | | | 2.02-1.97 (m, 1H), 1.94-1.87 (m, 1H), 1.81-1.75 (m, 1H), 1.68- |
| | | | | 1.63 (m, 2H), 1.62-1.59 (m, 1H), 1.54 (br d, J = 12.8 Hz, 3H), |
| | | | | 1.48-1.41 (m, 3H), 1.37 (br d, J = 6.8 Hz, 3H), 1.24 (br s, 3H), |
| | | | | 1.14 (br d, J = 9.2 Hz, 3H), 0.98-0.92 (m, 3H), 0.88 (s, 9H) |
| I-491^{q} | IX | IH | 1195.9 | 11.91-11.63 (m, 1H), 11.17-10.87 (m, 1H), 8.34 (d, J = 8.0 Hz, |
| | | | | 1H), 7.99-7.83 (m, 1H), 7.77 (br d, J = 9.2 Hz, 2H), 7.69 (br d, J = |
| | | | | 9.2 Hz, 1H), 7.60 (d, J = 8.4 Hz, 1H), 7.38 (br d, J = 13.2 Hz, |
| | | | | 4H), 7.22 (br d, J = 7.2 Hz, 1H), 7.03 (d, J = 8.4 Hz, 1H), 5.07 (d, |
| | | | | J = 3.6 Hz, 1H), 4.99 (br t, J = 7.2 Hz, 1H), 4.50 (d, J = 9.2 Hz, |
| | | | | 1H), 4.43 (t, J = 8.0 Hz, 1H), 4.25 (br s, 1H), 3.91 (s, 3H), 3.75 (s, |
| | | | | 2H), 3.65 (s, 3H), 3.58 (br s, 2H), 3.47-3.34 (m, 2H), 2.43 (s, 3H), |
| | | | | 2.42-2.35 (m, 3H), 2.25-2.17 (m, 4H), 2.12-1.98 (m, 2H), 1.94 |
| | | | | (br s, 3H), 1.76 (ddd, J = 4.4, 8.0, 12.8 Hz, 1H), 1.64 (br s, 4H), |
| | | | | 1.55 (br s, 10H), 1.49-1.38 (m, 5H), 1.35-1.27 (m, 2H), 0.92 (s, |
| | | | | 9H) |
| I-492^{q} | IX | IH | 1195.9 | 11.99-11.57 (m, 1H), 11.37-10.63 (m, 1H), 8.40 (d, J = 8.0 Hz, |
| | | | | 1H), 7.96-7.83 (m, 1H), 7.78 (br s, 1H), 7.68 (br d, J = 9.2 Hz, |
| | | | | 2H), 7.59 (s, 1H), 7.54 (d, J = 8.4 Hz, 1H), 7.42-7.29 (m, 3H), |
| | | | | 7.24-7.17 (m, 1H), 7.10 (d, J = 8.4 Hz, 1H), 5.09 (d, J = 3.6 Hz, |
| | | | | 1H), 4.98 (br t, J = 7.6 Hz, 1H), 4.50-4.43 (m, 2H), 4.34 (br d, J = |
| | | | | 1.2 Hz, 1H), 3.95 (s, 3H), 3.74 (s, 2H), 3.64 (s, 5H), 3.40 (br s, |
| | | | | 2H), 2.42 (s, 3H), 2.38 (br s, 3H), 2.19 (s, 4H), 2.13-2.07 (m, 1H), |
| | | | | 2.05-1.99 (m, 1H), 1.93 (br s, 4H), 1.64 (br s, 4H), 1.58 (br s, |
| | | | | 10H), 1.47-1.38 (m, 5H), 1.34-1.27 (m, 2H), 0.84 (s, 9H) |

^{a}The coupling was run from 1-4 hr at-10-25° C. DIEA also could be used for the base. Final compounds were purified via standard techniques including prep-HPLC and chromatography. DMF could also be used as a solvent. HOAt was also added when chiral coupling partners were employed to help suppress racemization.
^{b}LCMS data reported as M + 2H⁺.
^{c}LCMS data reported as M + Na⁺.
^{d}LCMS data reported as (M/2 + H)⁺.
^{e}After the coupling, LiOH•H₂O in THF, H₂O, and MeOH at 25-60° C. for 0.5-12 hr was used to hydrolyze the ester to the acid final product, which was purified via prep-HPLC.
^{f}The product of the coupling was deprotected with TFA in DCM at rt for 1-3 hr. The final compound was purified by prep-HPLC.
^{g}The product of the coupling was hydrolyzed with NaOH, in THF and H₂O at 25-50° C. for 2-4 h.
Then the final compound was purified by prep-HPLC.
ⁱECDI and HATU were used for the coupling in DMF at rt for 0.5-1 hr.
^{j}The product of the coupling was then deprotected with HCl in EtOAc at rt for 2-22 hrs. The final product was purified by prep-HPLC.
^{k}The product of the coupling was then deprotected with HCl/Dioxane in DCM at rt for 1-12 hr. The final product was purified by prep-HPLC.
^{l}EDCI was also added to the reaction for the coupling.
^{m}The coupling was heated to 50° C. for 12 hr.
ⁿThe product of the coupling was deprotected with CsF in DMSO at rt for 16 h.
^{o}The product of the coupling was further separated by SFC (column: REGIS (s, s) WHELK-O1 (250 mm*30 mm, 5 um); mobile phase: [ACN/IPA(0.1% NH3H2O)]; B %: 50%-50%, A9; 150 min) to give the diastereomers. The absolute stereochemistry was assigned arbitrarily.
^{p}The product of the coupling was further separated by SFC (column: DAICEL CHIRALCEL OX (250 mm*30 mm, 10 um); mobile phase: [ACN/MeOH(0.1% NH3H2O)]; B %: 60%-60%, A3; 30 min) to give the diastereomers. The absolute stereochemistry was assigned arbitrarily.
^{q}The product of the coupling was further separated by SFC (column: DAICEL CHIRALCEL OX (250 mm*30 mm, 10 um); mobile phase: [0.1% NH3H20 MEOH]; B %: 60%-60%, A2.75; 30 min), then re-purified by prep-HPLC (column: Phenomenex luna C18 150*25 mm*10 um; mobile phase: [water(FA)-ACN]; B %: 64%-94%, 10 min) to give the diastereomers. The absolute stereochemistry was assigned arbitrarily.

### Example 2 (Method 2); Syntheses of (2S,4R)—N-(2-(((1R,4S)-4-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl) cyclohexyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (I-114) and (2S,4R)—N-(2-(((1S,4R)-4-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl) cyclohexyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (I-115)

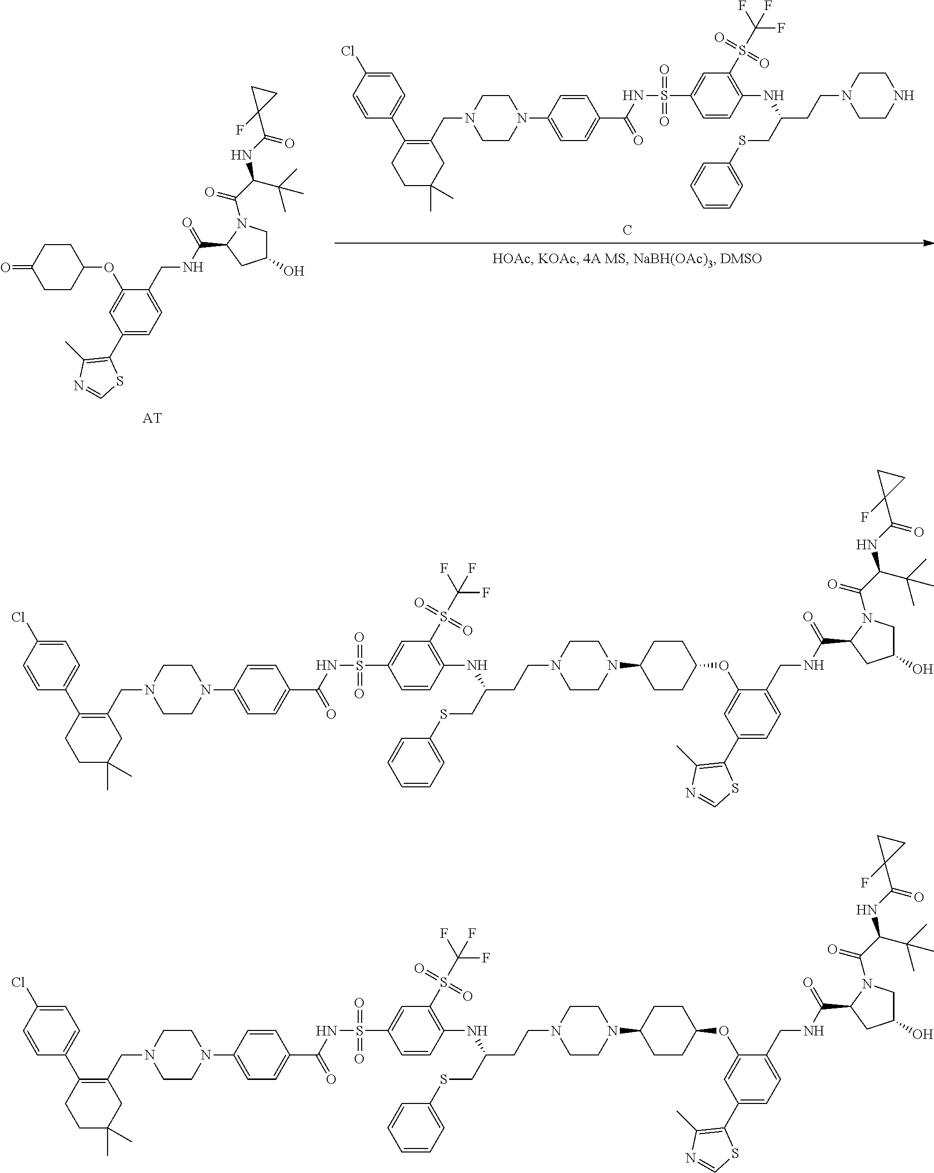

To a solution of (2S,4R)-1-((S)-2-(1-fluorocyclopropanecarboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide (80 mg, 130 umol, Intermediate AT) in THF (1 mL) and DMSO (1 mL) was added KOAc (37.5 mg, 382 umol), HOAc (22.9 mg, 382 umol), 4A molecular sieves (80 mg, 127.24 umol) and (R)-4-(4-((4′-chloro-4,4-dimethyl-3,4,5,6-tetrahydro-[1, l′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (124 mg, 127 umol, Intermediate C) and the mixture was stirred at 0° C., for 30 min. Then, NaBH (OAc); (80.9 mg, 382 umol) was added and the mixture was stirred at 0-20° C., for 12 h. On completion, the mixture was filtered, the filter liquor was concentrated under reduced pressure to give a residue. The residue was purified by prep-HPLC (TFA condition (column: Phenomenex luna C18 150*25 mm*10 um; mobile phase: [water (FA)-ACN]: B %: 32%-62%, 10 min) and basic condition (column: Phenomenex C18 150*25 mm*10 um; mobile phase: [water (NH₄HCO₃)-ACN]: B %: 56%-86%, 8 min)) to give 2S,4R)—N-(2-(((1R,4S)-4-(4-((R)-3-((4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl) cyclohexyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (8 mg, 4% yield) as a white solid (LC-MS (ESI⁺) m/z 1587.1 (M+H)⁺, ¹H NMR (400 MHZ, DMSO-d₆) δ=9.01-8.96 (m, 1H), 8.48-8.41 (m, 1H), 8.08-8.05 (m, 1H), 7.96-7.89 (m, 1H), 7.70 (br d, J=8.4 Hz, 2H), 7.42-7.32 (m, 6H), 7.29 (br t, J=7.6 Hz, 3H), 7.23-7.16 (m, 1H), 7.14-7.05 (m, 3H), 6.97-6.91 (m, 1H), 6.91-6.85 (m, 1H), 6.80-6.75 (m, 2H), 6.70 (br dd, J=0.8, 6.4 Hz, 1H), 5.17 (d, J=3.2 Hz, 1H), 4.62-4.56 (m, 1H), 4.53-4.48 (m, 1H), 4.40-4.32 (m, 2H), 4.30-4.14 (m, 2H), 4.07-3.98 (m, 1H), 3.67-3.55 (m, 4H), 3.15-3.07 (m, 4H), 2.78-2.63 (m, 8H), 2.45 (br s, 3H), 2.29-2.10 (m, 11H), 2.02-1.86 (m, 7H), 1.51-1.35 (m, 8H), 1.28-1.17 (m, 5H), 0.96 (br s, 15H)) and (2S,4R)—N-(2-(((1S,4R)-4-(4-((R)-3-((4-(N-(4-(4-((4′-chloro-4,4-dimethyl-3,4,5,6-tetrahydro-[1, l′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl) cyclohexyl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (12 mg, 6% yield) as a white solid. LC-MS (ESI⁺) m/z 1586.3 (M+H); (¹H NMR (400 MHZ, DMSO-d₆) ¿=8.98 (s, 1H), 8.50 (s, 1H), 8.05 (s, 1H), 7.99-7.90 (m, 1H), 7.72-7.65 (m, 2H), 7.50 (br d, J=1.2 Hz, 1H), 7.40-7.25 (m, 8H), 7.22-7.16 (m, 1H), 7.13-7.08 (m, 2H), 7.03-6.97 (m, 1H), 6.97-6.85 (m, 2H), 6.81-6.63 (m, 3H), 5.18 (d, J=3.6 Hz, 1H), 4.73 (dt, J=2.4, 8.8 Hz, 1H), 4.63-4.50 (m, 2H), 4.35 (br s, 2H), 4.31-4.23 (m, 1H), 4.10-3.97 (m, 2H), 3.67-3.56 (m, 4H), 3.18-3.03 (m, 5H), 2.78-2.69 (m, 3H), 2.68-2.66 (m, 2H), 2.45 (s, 3H), 2.36-2.30 (m, 2H), 2.29-2.17 (m, 7H), 2.13-1.87 (m, 8H), 1.81-1.54 (m, 6H), 1.48-1.37 (m, 4H), 1.29-1.19 (m, 5H), 0.95 (s, 15H)). Absolute stereochemistry of the diastereomers was arbitrarily assigned.

TABLE 4
Compounds synthesized via Method 2, using the corresponding
amines and aldehydes/ketones for the reductive amination.

| | | | LCMS | |
|---|---|---|---|---|
| | | | (ESI+) | |
| | | Aldehyde/ | m/z | |
| I-# | Amine | Ketone | (M + H)⁺ | ¹H NMR (400 MHz, DMSO-d6) |
| I-116^{a} | CE | CG | 1486.8 | 8.35 (br d, J = 8.4 Hz, 1H), 8.15 (br d, J = 3.5 Hz, 1H), 8.02- |
| | | | | 7.94 (m, 1H), 7.86-7.77 (m, 1H), 7.76-7.70 (m, 2H), 7.40 (br |
| | | | | t, J = 8.6 Hz, 4H), 7.34-7.29 (m, 4H), 7.26 (br t, J = 7.5 Hz, |
| | | | | 7H), 6.90 (br d, J = 7.5 Hz, 2H), 5.13-5.05 (m, 1H), 4.89- |
| | | | | 4.80 (m, 1H), 4.78-4.70 (m, 1H), 4.51 (br d, J = 9.3 Hz, 1H), |
| | | | | 4.47-4.41 (m, 1H), 4.30-4.23 (m, 1H), 4.17-4.08 (m, 2H), |
| | | | | 3.91-3.84 (m, 1H), 3.67-3.47 (m, 7H), 3.24-2.96 (m, 8H), |
| | | | | 2.31-2.20 (m, 8H), 2.17-1.92 (m, 8H), 1.86-1.71 (m, 2H), |
| | | | | 1.68-1.58 (m, 1H), 1.51-1.40 (m, 6H), 1.30-1.15 (m, 6H), |
| | | | | 0.99 (br s, 6H), 0.93 (s, 9H). |

^{a}No HPLC separation required. The product of the coupling was deprotected using CsF in DMSO at rt for 1 hr. Then the final compound was purified by prep-HPLC

### Example 3 (Method 3); Synthesis of 2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)-N-(6-(R)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexyl)sulfonyl)thiazole-4-carboxamide (I-26)

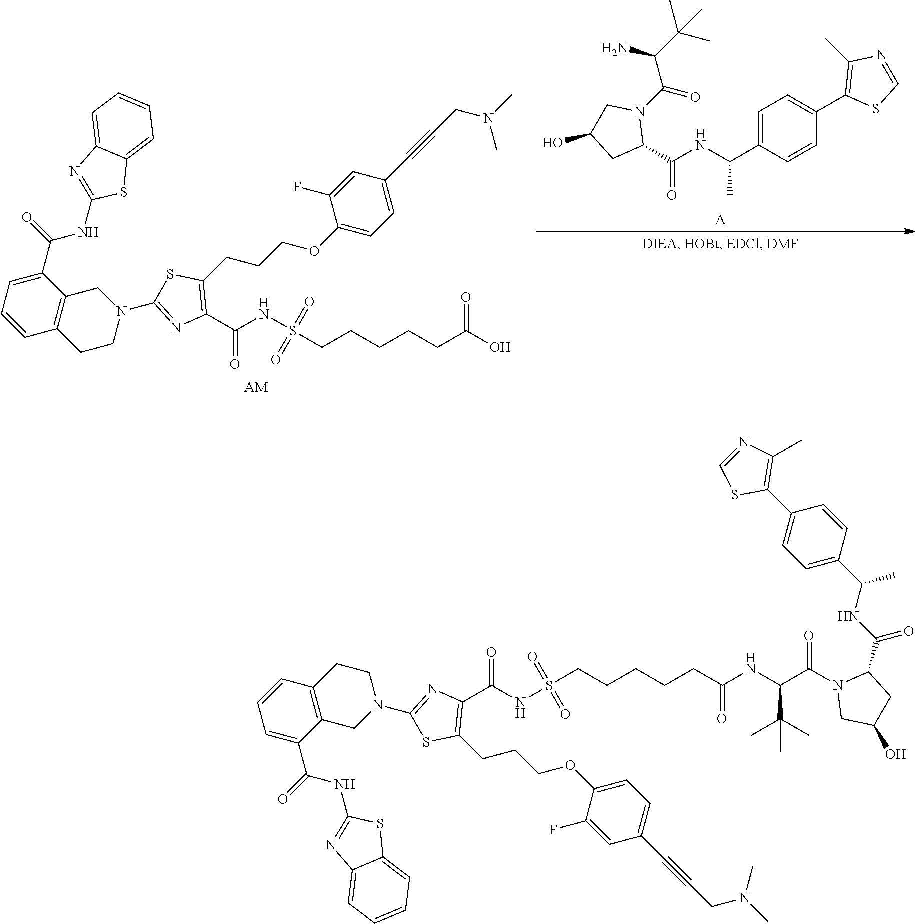

A solution of 6-(N-(2-(8-(benzo[d]thiazol-2-ylcarbamoyl)-3,4-dihydroisoquinolin-2 (1H)-yl)-5-(3-(4-(3-(dimethylamino)prop-1-yn-1-yl)-2-fluorophenoxy)propyl)thiazole-4-carbonyl)sulfamoyl)hexanoic acid (90.0 mg, 106 umol, Intermediate AM), (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (63.9 mg, 133 umol, HCl salt, Intermediate A), DIEA (57.2 mg, 443 umol), HOBt (17.9 mg, 133 umol) and EDCI (25.5 mg, 133 umol) in DMF (1 mL) was stirred at 25° C., for 24 h. On completion, the reaction mixture was filtered and concentrated under reduced pressure to give a residue. The residue was purified by prep-HPLC (Phenomenex luna C18 150*25 mm*10 um; mobile phase: [water (FA)-ACN]; B %: 35%-65%, 10 min) to give the title compound (10.4 mg, 9% yield) as white solid. LC-MS (ESI⁺) m/z 1273.9 (M+H)⁺. ¹H NMR (400 MHZ, DMSO-d₆) δ=8.98 (s, 1H), 8.38 (br d, J=7.6 Hz, 1H), 8.04 (br d, J=7.6 Hz, 1H), 7.83-7.78 (m, 2H), 7.68 (br d, J=7.6 Hz, 1H), 7.50-7.40 (m, 6H), 7.40-7.35 (m, 4H), 7.25 (br d, J=11.6 Hz, 1H), 7.19-7.11 (m, 2H), 5.19-5.03 (m, 1H), 4.94-4.89 (m, 1H), 4.80 (br s, 2H), 4.50 (br d, J=9.2 Hz, 1H), 4.42 (br t, J=8.0 Hz, 1H), 4.27 (br s, 1H), 4.08 (br t, J=6.0 Hz, 2H), 3.83 (br s, 2H), 3.59 (br s, 2H), 3.20-3.16 (m, 4H), 3.12 (br s, 1H), 3.03 (br s, 2H), 2.45 (s, 3H), 2.23 (s, 6H), 2.14-2.09 (m, 1H), 2.01 (br d, J=7.6 Hz, 4H), 1.84-1.72 (m, 2H), 1.66-1.60 (m, 2H), 1.47 (br d, J=7.2 Hz, 2H), 1.37 (br d, J=6.8 Hz, 3H), 1.34-1.29 (m, 2H), 0.92 (s, 9H).

TABLE 5
Compounds synthesized via Method 3, using the corresponding amines and acids for the coupling.

| | | | LCMS | |
|---|---|---|---|---|
| | | | (ESI+) | |
| | | | m/z | |
| I-#^{a} | Amine | Acid | (M + H)⁺ | 1H NMR (400 MHz, DMSO-d6) |
| I-27 | A | AO | 658.8^{b} | 8.98 (s, 1H), 8.36 (d, J = 8.0 Hz, 1H), 8.03 (d, J = 8.0 Hz, 1H), 7.80- |
| | | | | 7.74 (m, 2H), 7.68 (br d, J = 7.6 Hz, 1H), 7.49-7.41 (m, 5H), 7.40- |
| | | | | 7.32 (m, 5H), 7.25 (dd, J = 1.6, 12.0 Hz, 1H), 7.18 (br d, J = 9.2 |
| | | | | Hz, 1H), 7.13-7.08 (m, 1H), 4.91 (br t, J = 7.2 Hz, 1H), 4.80 (s, |
| | | | | 2H), 4.50 (br d, J = 9.2 Hz, 1H), 4.41 (br t, J = 7.6 Hz, 1H), 4.26 |
| | | | | (br d, J = 3.2 Hz, 1H), 4.07 (br t, J = 6.4 Hz, 2H), 3.87 (br t, J = 5.6 |
| | | | | Hz, 2H), 3.59 (br s, 2H), 3.47 (s, 4H), 3.17 (s, 2H), 3.03 (br t, J = |
| | | | | 5.6 Hz, 2H), 2.45 (s, 3H), 2.26 (s, 6H), 2.06-1.97 (m, 4H), 1.81- |
| | | | | 1.75 (m, 1H), 1.67-1.61 (m, 2H), 1.48-1.42 (m, 2H), 1.36 (br d, |
| | | | | J = 7.2 Hz, 3H), 1.32 (br d, J = 2.0 Hz, 2H), 1.23 (br s, 2H), 1.20 |
| | | | | (br s, 6H), 0.92 (s, 9H) |
| I-117 | A | BJ | 1235.9 | 12.97-12.74 (m, 1H), 11.95-11.57 (m, 1H), 8.98 (s, 1H), 8.42- |
| | | | | 8.31 (m, 1H), 8.03 (d, J = 7.6 Hz, 1H), 7.93-7.84 (m, 1H), 7.79 (d, |
| | | | | J = 7.2 Hz, 1H), 7.63 (d, J = 6.4 Hz, 1H), 7.54-7.42 (m, 5H), 7.39- |
| | | | | 7.27 (m, 5H), 7.03-6.90 (m, 1H), 5.09 (d, J = 2.8 Hz, 1H), 4.96- |
| | | | | 4.88 (m, 3H), 4.54-4.40 (m, 2H), 4.28 (d, J = 1.2 Hz, 1H), 3.94 (s, |
| | | | | 2H), 3.70 (s, 2H), 3.61 (s, 2H), 3.02 (s, 2H), 2.45 (s, 3H), 2.27 (d, |
| | | | | J = 15.4 Hz, 1H), 2.11 (s, 3H), 2.03-1.97 (m, 1H), 1.92 (s, 4H), 1.83 |
| | | | | (dd, J = 4.0, 8.5 Hz, 2H), 1.66-1.48 (m, 14H), 1.37 (d, J = 6.8 Hz, |
| | | | | 3H), 1.23 (s, 1H), 0.93 (s, 9H) |
| I-118 | A | CR | 1210.7 | 13.00-12.70 (m, 1H), 11.88-11.59 (m, 1H), 8.98 (s, 1H), 8.37 (br |
| | | | | d, J = 8.0 Hz, 1H), 8.03 (d, J = 8.0 Hz, 1H), 7.80 (br t, J = 9.2 Hz, |
| | | | | 2H), 7.63 (br d, J = 7.6 Hz, 1H), 7.48 (br d, J = 7.6 Hz, 1H), 7.45- |
| | | | | 7.42 (m, 3H), 7.39-7.33 (m, 5H), 7.27 (br s, 1H), 7.02-6.93 (m, |
| | | | | 1H), 5.10 (d, J = 3.6 Hz, 1H), 4.94 (br s, 2H), 4.51 (br d, J = 9.6 |
| | | | | Hz, 1H), 4.42 (br t, J = 8.0 Hz, 1H), 4.27 (br s, 1H), 3.95-3.92 (m, |
| | | | | 2H), 3.84 (br d, J = 7.2 Hz, 2H), 3.60 (br s, 2H), 3.25 (br s, 2H), |
| | | | | 3.03-3.00 (m, 2H), 2.45 (s, 3H), 2.25-2.21 (m, 1H), 2.11 (s, 3H), |
| | | | | 1.99 (br dd, J = 4, 6.4 Hz, 1H), 1.83-1.74 (m, 3H), 1.66-1.59 (m, |
| | | | | 4H), 1.53 (br d, J = 12.8 Hz, 4H), 1.47-1.43 (m, 2H), 1.37 (br d, |
| | | | | J = 7.2 Hz, 4H), 1.31-1.26 (m, 2H), 1.17-1.11 (m, 4H), 0.93 (s, 9H) |
| I-119 | A | DA | 1036.2 | 12.98-12.71 (m, 1H), 11.38-11.03 (m, 1H), 8.98 (s, 1H), 8.37 (br |
| | | | | d, J = 8.0 Hz, 1H), 8.02 (d, J = 8.0 Hz, 1H), 7.82-7.77 (m, 2H), |
| | | | | 7.73 (br s, 1H), 7.62 (br d, J = 7.6 Hz, 1H), 7.47-7.42 (m, 4H), |
| | | | | 7.39-7.35 (m, 4H), 7.30 (br d, J = 7.2 Hz, 1H), 7.10-7.00 (m, 1H), |
| | | | | 5.09 (br s, 1H), 4.93 (s, 2H), 4.50 (br d, J = 9.2 Hz, 1H), 4.42 (t, |
| | | | | J = 8.0 Hz, 1H), 4.27 (br s, 1H), 4.02 (br t, J = 5.2 Hz, 2H), 3.59 (br |
| | | | | s, 2H), 3.43-3.39 (m, 2H), 3.01 (br t, J = 5.6 Hz, 2H), 2.45 (s, 3H), |
| | | | | 2.25-2.19 (m, 1H), 2.12-2.07 (m, 1H), 2.01 (br d, J = 10.0 Hz, |
| | | | | 1H), 1.80-1.75 (m, 1H), 1.67 (br s, 2H), 1.51-1.42 (m, 3H), 1.38- |
| | | | | 1.32 (m, 5H), 0.92 (s, 9H) |
| I-120 | A | DD | 1172.4 | 8.98 (s, 1H), 8.37 (br d, J = 7.6 Hz, 1H), 7.90-7.84 (m, 1H), 7.82- |
| | | | | 7.73 (m, 2H), 7.63 (br dd, J = 4.4, 8.4 Hz, 1H), 7.45-7.33 (m, |
| | | | | 7H), 7.21 (br t, J = 7.2 Hz, 1H), 5.09 (br d, J = 2.8 Hz, 1H), 4.96- |
| | | | | 4.85 (m, 1H), 4.50 (br d, J = 9.2 Hz, 1H), 4.42 (br t, J = 7.6 Hz, |
| | | | | 1H), 4.27 (br s, 1H), 3.88 (br d, J = 7.2 Hz, 2H), 3.66-3.58 (m, |
| | | | | 5H), 2.37 (br s, 3H), 2.20 (br s, 3H), 2.08 (br t, J = 6.4 Hz, 1H), |
| | | | | 2.05-1.95 (m, 2H), 1.83-1.74 (m, 2H), 1.66 (br s, 3H), 1.61-1.50 |
| | | | | (m, 5H), 1.47-1.41 (m, 2H), 1.36 (br d, J = 6.8 Hz, 3H), 1.31-1.21 |
| | | | | (m, 5H), 1.16 (br d, J = 8.0 Hz, 3H), 1.04-0.95 (m, 3H), 0.92 (s, 9H) |
| I-121^{c} | DI | BI | 1323.6 | 12.91-12.76 (m, 1H), 11.77 (s, 1H), 8.97 (s, 1H), 8.48 (br t, J = 6.0 |
| | | | | Hz, 1H), 8.01 (d, J = 8.0 Hz, 1H), 7.77 (d, J = 8.0 Hz, 1H), 7.62 (br |
| | | | | d, J = 7.2 Hz, 1H), 7.52 (d, J = 8.8 Hz, 1H), 7.45 (s, 1H), 7.43-7.39 |
| | | | | (m, 2H), 7.38-7.32 (m, 2H), 7.31-7.28 (m, 1H), 7.26 (s, 1H), 6.97 |
| | | | | (d, J = 8.8 Hz, 1H), 6.95-6.90 (m, 2H), 4.95 (s, 2H), 4.59 (d, J = |
| | | | | 9.2 Hz, 1H), 4.52 (t, J = 8.4 Hz, 1H), 4.36-4.32 (m, 1H), 4.31- |
| | | | | 4.25 (m, 1H), 4.21-4.14 (m, 1H), 4.01-3.96 (m, 2H), 3.91 (br t, J = |
| | | | | 5.6 Hz, 2H), 3.68 (s, 2H), 3.35-3.28 (m, 3H), 3.02-2.98 (m, |
| | | | | 2H), 2.54 (s, 2H), 2.43 (s, 3H), 2.11 (s, 3H), 2.08-2.04 (m, 1H), |
| | | | | 1.96-1.91 (m, 1H), 1.88 (br s, 3H), 1.74-1.65 (m, 4H), 1.62-1.57 |
| | | | | (m, 3H), 1.53 (br s, 3H), 1.49 (br s, 7H), 1.39 (br s, 2H), 1.35-1.32 |
| | | | | (m, 1H), 1.21 (br dd, J = 2.4, 8.8 Hz, 2H), 0.95 (s, 9H) |
| I-122^{c, d} | DK | BI | 1365.2 | 13.00-12.69 (m, 1H), 11.92-11.46 (m, 1H), 8.97 (s, 1H), 8.49 (br |
| | | | | t, J = 6.0 Hz, 1H), 8.01 (d, J = 8.0 Hz, 1H), 7.78 (d, J = 8.0 Hz, 1H), |
| | | | | 7.63 (br d, J = 7.6 Hz, 1H), 7.52-7.45 (m, 2H), 7.44-7.39 (m, 2H), |
| | | | | 7.37-7.33 (m, 2H), 7.31-7.28 (m, 1H), 7.27 (s, 1H), 6.97-6.92 |
| | | | | (m, 3H), 5.16 (d, J = 3.2 Hz, 1H), 4.95 (s, 2H), 4.59 (d, J = 9.2 Hz, |
| | | | | 1H), 4.51 (t, J = 8.0 Hz, 1H), 4.35-4.27 (m, 2H), 4.22-4.15 (m, |
| | | | | 1H), 4.01 (br t, J = 6.0 Hz, 2H), 3.92 (br t, J = 6.0 Hz, 2H), 3.68 (s, |
| | | | | 2H), 3.65-3.58 (m, 2H), 3.24-3.20 (m, 2H), 3.03-2.99 (m, 2H), |
| | | | | 2.44 (s, 3H), 2.11 (s, 3H), 1.92-1.87 (m, 4H), 1.75-1.70 (m, 2H), |
| | | | | 1.63-1.59 (m, 4H), 1.54 (br s, 2H), 1.50 (br s, 6H), 1.43-1.37 (m, |
| | | | | 4H), 1.32-1.20 (m, 11H), 0.95 (s, 9H) |
| I-123 | A | DM | 1245.2 | 13.06-12.66 (m, 1H), 12.27-11.86 (m, 1H), 8.98 (s, 1H), 8.37 (br |
| | | | | d, J = 7.6 Hz, 1H), 8.03 (d, J = 7.6 Hz, 1H), 7.93 (br d, J = 9.2 Hz, |
| | | | | 1H), 7.83-7.74 (m, 2H), 7.63 (br d, J = 6.4 Hz, 2H), 7.49-7.32 |
| | | | | (m, 11H), 7.17-6.95 (m, 2H), 5.12 (d, J = 3.2 Hz, 1H), 4.99-4.87 |
| | | | | (m, 3H), 4.53 (br d, J = 9.4 Hz, 1H), 4.44 (s, 1H), 4.28 (br s, 1H), |
| | | | | 3.92 (br s, 2H), 3.74 (br d, J = 7.2 Hz, 2H), 3.61 (br s, 2H), 3.01 (br |
| | | | | s, 2H), 2.92-2.78 (m, 2H), 2.45 (s, 3H), 2.07-1.93 (m, 2H), 1.85- |
| | | | | 1.72 (m, 4H), 1.70-1.58 (m, 4H), 1.56-1.42 (m, 3H), 1.37 (br d, |
| | | | | J = 6.8 Hz, 3H), 1.15 (br d, J = 8.0 Hz, 3H), 0.88 (br s, 11H) |
| I-124 | A | DN | 1076.7 | 8.98 (s, 1H), 8.36 (br d, J = 8.0 Hz, 1H), 8.18-8.07 (m, 1H), 7.91- |
| | | | | 7.81 (m, 3H), 7.71 (br d, J = 8.4 Hz, 2H), 7.45-7.35 (m, 9H), 7.11 |
| | | | | (br d, J = 8.4 Hz, 2H), 6.87 (br d, J = 8.8 Hz, 2H), 5.11 (br d, J = |
| | | | | 2.8 Hz, 1H), 4.91 (br t, J = 7.2 Hz, 1H), 4.51-4.39 (m, 2H), 4.27 |
| | | | | (br s, 1H), 3.59 (br s, 2H), 3.23 (br s, 3H), 2.91-2.84 (m, 2H), 2.78 |
| | | | | (br s, 2H), 2.45 (s, 3H), 2.29 (br s, 4H), 2.22 (br s, 3H), 1.99 (br s, |
| | | | | 3H), 1.82-1.74 (m, 1H), 1.43 (br t, J = 5.6 Hz, 3H), 1.36 (br d, J = |
| | | | | 7.2 Hz, 3H), 1.24 (br s, 1H), 0.96 (s, 6H), 0.81 (s, 9H) |
| I-310 | A | EK | 1288.6 | 8.98 (s, 1H), 8.38 (d, J = 8.0 Hz, 1H), 8.13 (s, 1H), 7.86 (br d, J = |
| | | | | 9.6 Hz, 1H), 7.80 (br dd, J = 3.2, 5.2 Hz, 1H), 7.67-7.54 (m, 4H), |
| | | | | 7.44-7.42 (m, 2H), 7.38-7.32 (m, 4H), 7.27 (s, 1H), 7.17-7.14 |
| | | | | (m, 1H), 6.99 (br d, J = 6.0 Hz, 2H), 5.11 (d, J = 3.6 Hz, 1H), 4.91 |
| | | | | (t, J = 7.2 Hz, 1H), 4.51 (d, J = 9.2 Hz, 1H), 4.44-4.40 (m, 1H), |
| | | | | 4.27 (br s, 1H), 4.06 (br s, 2H), 3.96-3.91 (m, 2H), 3.77 (br d, J = |
| | | | | 7.2 Hz, 2H), 3.60-3.57 (m, 2H), 2.86 (br t, J = 5.6 Hz, 3H), 2.45 |
| | | | | (s, 3H), 2.32-2.31 (m, 3H), 2.03-1.97 (m, 4H), 1.90-1.82 (m, |
| | | | | 4H), 1.78 (br dd, J = 4.2, 8.8 Hz, 3H), 1.68 (br s, 4H), 1.64 (br s, |
| | | | | 3H), 1.53 (br d, J = 13.6 Hz, 2H), 1.37 (d, J = 7.2 Hz, 3H), 1.25- |
| | | | | 1.19 (m, 2H), 1.18-1.15 (m, 2H), 0.94 (s, 9H) |
| I-318 | M | EM | 1191.6 | 12.88 (br s, 1H), 12.12 (s, 1H), 8.98 (s, 1H), 8.56 (t, J = 6.0 Hz, 1H), |
| | | | | 8.02 (t, J = 9.3 Hz, 2H), 7.84-7.72 (m, 3H), 7.64 (br d, J = 7.5 Hz, |
| | | | | 1H), 7.52-7.42 (m, 4H), 7.42-7.32 (m, 7H), 7.11 (s, 1H), 6.98 (d, |
| | | | | J = 9.0 Hz, 1H), 5.02-4.94 (m, 2H), 4.54 (d, J = 9.5 Hz, 1H), 4.48- |
| | | | | 4.38 (m, 2H), 4.34 (br s, 1H), 4.26-4.14 (m, 1H), 3.94 (br t, J = |
| | | | | 5.5 Hz, 2H), 3.74 (br d, J = 7.5 Hz, 2H), 3.68-3.62 (m, 2H), 3.04- |
| | | | | 2.98 (m, 2H), 2.92-2.82 (m, 2H), 2.70-2.56 (m, 2H), 2.44 (s, 3H), |
| | | | | 2.04-1.96 (m, 2H), 1.90 (ddd, J = 4.8, 8.5, 12.8 Hz, 1H), 1.78 (s, |
| | | | | 3H), 1.24 (br s, 1H), 0.86 (s, 9H), 0.84-0.78 (m, 6H) |
| I-320 | EP | ER | 1249.2 | 12.88 (br s, 1H), 12.46-12.31 (m, 1H), 8.98 (s, 1H), 8.83 (br d, J = |
| | | | | 2.0 Hz, 1H), 8.57 (s, 1H), 8.05-8.00 (m, 2H), 7.80 (br d, J = 7.6 |
| | | | | Hz, 1H), 7.66-7.61 (m, 1H), 7.49-7.42 (m, 5H), 7.41-7.33 (m, |
| | | | | 6H), 7.08 (s, 1H), 7.02-6.91 (m, 1H), 5.15 (d, J = 3.6 Hz, 1H), 4.95 |
| | | | | (br s, 2H), 4.55 (d, J = 9.6 Hz, 1H), 4.46-4.40 (m, 2H), 4.35 (br s, |
| | | | | 1H), 4.25-4.17 (m, 1H), 3.92 (br t, J = 5.2 Hz, 2H), 3.75 (br d, J = |
| | | | | 7.2 Hz, 2H), 3.66 (br d, J = 3.6 Hz, 2H), 3.04-3.00 (m, 3H), 2.80- |
| | | | | 2.74 (m, 1H), 2.44 (s, 3H), 2.03 (br s, 2H), 1.95-1.86 (m, 2H), 1.80 |
| | | | | (br s, 3H), 1.70-1.64 (m, 3H), 1.62 (br d, J = 4.0 Hz, 1H), 1.54- |
| | | | | 1.44 (m, 3H), 1.24 (s, 1H), 1.19-1.13 (m, 3H), 0.89 (s, 9H) |
| I-348 | A | EX | 1286.2 | 12.92-12.80 (m, 1H), 11.89-11.74 (m, 1H), 8.98 (s, 1H), 8.38 (d, |
| | | | | J = 8.0 Hz, 1H), 8.03 (d, J = 7.2 Hz, 1H), 7.88-7.84 (m, 1H), 7.81- |
| | | | | 7.77 (m, 1H), 7.64-7.60 (m, 2H), 7.50-7.47 (m, 1H), 7.45-7.41 |
| | | | | (m, 4H), 7.39-7.33 (m, 4H), 7.26-7.17 (m, 1H), 7.13 (br s, 1H), |
| | | | | 7.00-6.85 (m, 3H), 5.32 (t, J = 4.4 Hz, 1H), 5.10 (d, J = 3.6 Hz, |
| | | | | 1H), 4.96-4.89 (m, 3H), 4.51 (d, J = 9.2 Hz, 1H), 4.47-4.42 (m, |
| | | | | 1H), 4.32-4.24 (m, 1H), 3.93 (br s, 2H), 3.84-3.81 (m, 2H), 3.59 |
| | | | | (br d, J = 4.0 Hz, 1H), 3.01 (br s, 2H), 2.88-2.77 (m, 2H), 2.64- |
| | | | | 2.59 (m, 1H), 2.45 (s, 3H), 2.44 (br s, 1H), 2.04-1.97 (m, 4H), 1.83- |
| | | | | 1.78 (m, 3H), 1.61-1.57 (m, 4H), 1.53-1.49 (m, 2H), 1.38 (d, J = |
| | | | | 7.2 Hz, 3H), 1.23 (br s, 6H), 0.95 (s, 9H) |
| I-349 | A | FD | 1288.6 | 12.98-12.79 (m, 1H), 11.93-11.67 (m, 1H), 8.98 (s, 1H), 8.38 (d, |
| | | | | J = 8.0 Hz, 1H), 8.03 (d, J = 8.0 Hz, 1H), 7.85 (br d, J = 9.6 Hz, |
| | | | | 1H), 7.79 (br d, J = 8.0 Hz, 1H), 7.67-7.59 (m, 3H), 7.50-7.41 |
| | | | | (m, 5H), 7.41-7.32 (m, 4H), 7.15 (s, 1H), 7.02-6.86 (m, 3H), 5.10 |
| | | | | (d, J = 3.6 Hz, 1H), 4.99-4.88 (m, 3H), 4.51 (d, J = 9.2 Hz, 1H), |
| | | | | 4.47-4.40 (m, 1H), 4.28 (br s, 1H), 3.97-3.84 (m, 4H), 3.79 (br d, |
| | | | | J = 7.6 Hz, 2H), 3.65-3.54 (m, 2H), 3.02 (br t, J = 5.6 Hz, 2H), |
| | | | | 2.90-2.75 (m, 2H), 2.64 (br dd, J = 9.6, 11.2 Hz, 1H), 2.46-2.44 |
| | | | | (m, 3H), 2.05-1.96 (m, 1H), 1.83-1.76 (m, 4H), 1.72-1.64 (m, |
| | | | | 2H), 1.63-1.49 (m, 2H), 1.40-1.33 (m, 4H), 1.26-1.20 (m, 4H), |
| | | | | 0.97-0.91 (m, 9H), 0.83 (t, J = 7.2 Hz, 6H) |
| I-423^{c} | FW | FP | 1011.9 | 11.94-11.77 (m, 1H), 11.07 (s, 1H), 7.94-7.83 (m, 2H), 7.75 (br |
| | | | | d, J = 7.6 Hz, 1H), 7.57 (br s, 1H), 7.43-7.34 (m, 3H), 7.23 (br t, |
| | | | | J = 7.6 Hz, 1H), 6.96-6.84 (m, 2H), 6.77 (d, J = 7.2 Hz, 1H), 5.43- |
| | | | | 5.21 (m, 1H), 3.73 (br s, 2H), 3.64 (s, 3H), 3.48 (s, 3H), 3.39 (br d, |
| | | | | J = 7.2 Hz, 2H), 2.85 (br d, J = 14.8 Hz, 1H), 2.79 (br s, 2H), 2.67 |
| | | | | (br d, J = 2.0 Hz, 1H), 2.58 (br d, J = 3.2 Hz, 1H), 2.40 (s, 3H), |
| | | | | 2.21-2.15 (m, 3H), 1.92 (br s, 4H), 1.64 (br d, J = 11.4 Hz, 5H), |
| | | | | 1.58-1.50 (m, 11H), 1.47-1.38 (m, 2H) |
| I-424^{c} | FZ | FP | 1011.5 | 12.00-11.61 (m, 1H), 11.07 (s, 1H), 7.94-7.76 (m, 2H), 7.70 (br |
| | | | | d, J = 8.4 Hz, 2H), 7.43-7.28 (m, 3H), 7.21 (br t, J = 7.2 Hz, 1H), |
| | | | | 6.94-6.87 (m, 2H), 6.75 (d, J = 8.4 Hz, 1H), 5.30 (br dd, J = 5.2, |
| | | | | 12.8 Hz, 1H), 3.74-3.61 (m, 5H), 3.27 (s, 3H), 2.94-2.83 (m, 1H), |
| | | | | 2.75-2.53 (m, 3H), 2.48-2.43 (m, 2H), 2.39 (br s, 3H), 2.18 (s, |
| | | | | 3H), 2.00-1.89 (m, 4H), 1.68-1.43 (m, 17H), 1.34-1.27 (m, 2H) |
| I-425^{c} | FZ | CA | 997.8 | 12.99-12.75 (m, 1H), 11.91-11.66 (m, 1H), 11.07 (s, 1H), 8.01 |
| | | | | (d, J = 7.6 Hz, 1H), 7.79 (d, J = 8.0 Hz, 1H), 7.62 (br d, J = 7.2 Hz, |
| | | | | 1H), 7.55-7.42 (m, 2H), 7.41-7.32 (m, 3H), 7.28 (br s, 1H), 7.00- |
| | | | | 6.96 (m, 2H), 6.83 (br d, J = 8.4 Hz, 1H), 5.32 (br dd, J = 5.2, 12.8 |
| | | | | Hz, 1H), 4.94 (br s, 2H), 3.92 (br t, J = 5.6 Hz, 2H), 3.81 (br d, J = |
| | | | | 7.2 Hz, 2H), 3.30 (br s, 1H), 3.28 (s, 3H), 3.02-2.91 (m, 2H), 2.90- |
| | | | | 2.83 (m, 1H), 2.74-2.52 (m, 5H), 2.11 (s, 3H), 2.01-1.95 (m, |
| | | | | 1H), 1.80-1.72 (m, 1H), 1.69-1.44 (m, 10H), 1.39-1.30 (m, 2H), |
| | | | | 1.15-1.06 (m, 3H), 0.98-0.88 (m, 2H) |
| I-426^{c} | FP | CA | 997.6 | 12.90-12.78 (m, 1H), 11.81-11.73 (m, 1H), 11.11-11.03 (m, 1H), |
| | | | | 8.00 (d, J = 8.0 Hz, 1H), 7.78 (br d, J = 7.6 Hz, 1H), 7.62 (br d, J = |
| | | | | 7.2 Hz, 1H), 7.52-7.44 (m, 2H), 7.41-7.32 (m, 3H), 7.27 (br d, J = |
| | | | | 0.8 Hz, 1H), 6.99-6.90 (m, 3H), 6.83 (dd, J = 1.2, 7.4 Hz, 1H), |
| | | | | 5.33 (s, 1H), 4.95 (br s, 2H), 3.91 (br t, J = 6.0 Hz, 2H), 3.81 (br d, |
| | | | | J = 7.2 Hz, 2H), 3.51 (s, 3H), 3.00-2.96 (m, 2H), 2.86-2.82 (m, |
| | | | | 2H), 2.74 (br s, 1H), 2.70 (br d, J = 4.0 Hz, 1H), 2.68-2.66 (m, |
| | | | | 1H), 2.63 (br s, 2H), 2.11 (s, 3H), 2.00-1.95 (m, 1H), 1.82-1.71 |
| | | | | (m, 2H), 1.63 (br d, J = 3.6 Hz, 4H), 1.57-1.51 (m, 3H), 1.50-1.43 |
| | | | | (m, 3H), 1.23 (br s, 1H), 1.11 (br d, J = 8.4 Hz, 2H), 0.99-0.89 (m, 2H) |
| I-446 | EO | GX | 1138.6 | 8.55 (br t, J = 6.8 Hz, 1H), 8.35 (br d, J = 3.2 Hz, 1H), 8.14 (s, 1H), |
| | | | | 8.03-7.6 (m, 1H), 7.84 (br d, J = 9.2 Hz, 1H), 7.80-7.73 (m, 1H), |
| | | | | 7.65-7.47 (m, 2H), 7.44-7.40 (m, 1H), 7.38-7.28 (m, 5H), 5.11 |
| | | | | (d, J = 3.2 Hz, 1H), 4.52 (br d, J = 9.2 Hz, 1H), 4.43-4.36 (m, 2H), |
| | | | | 4.33 (br s, 1H), 4.18 (br d, J = 5.6 Hz, 1H), 4.15-4.12 (m, 1H), |
| | | | | 3.73 (s, 2H), 3.64 (br s, 5H), 2.37 (br s, 3H), 2.23 (br s, 4H), 2.10 |
| | | | | (br d, J = 14.4 Hz, 1H), 2.05-1.97 (m, 2H), 1.93 (br s, 3H), 1.89- |
| | | | | 1.84 (m, 1H), 1.68-1.63 (m, 3H), 1.60-1.54 (m, 11H), 1.47-1.41 |
| | | | | (m, 2H), 1.31-1.22 (m, 3H), 0.92 (s, 9H) |

^{a}The reaction was run at rt for 2-24 hrs. The final compounds were purified via prep-HPLC and chromatography. DCM could also be used as the solvent.
^{b}LCMS data reported as (M/2 + H)⁺.
^{c}EDCI, DMAP, and DCM was used for the coupling at at 25-40° C. for 2-36 hrs. The product of the coupling was further deprotected with CsF in DMSO at rt for 12 hrs. The final compound was purified by prep-HPLC.

### Example 4 (Method 4); Synthesis of 6-(8-(benzo[d]thiazol-2-ylcarbamoyl)-3,4-dihydroisoquinolin-2 (1H)-yl)-3-(6-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)amino)-6-oxohexyl)picolinic acid (1-36)

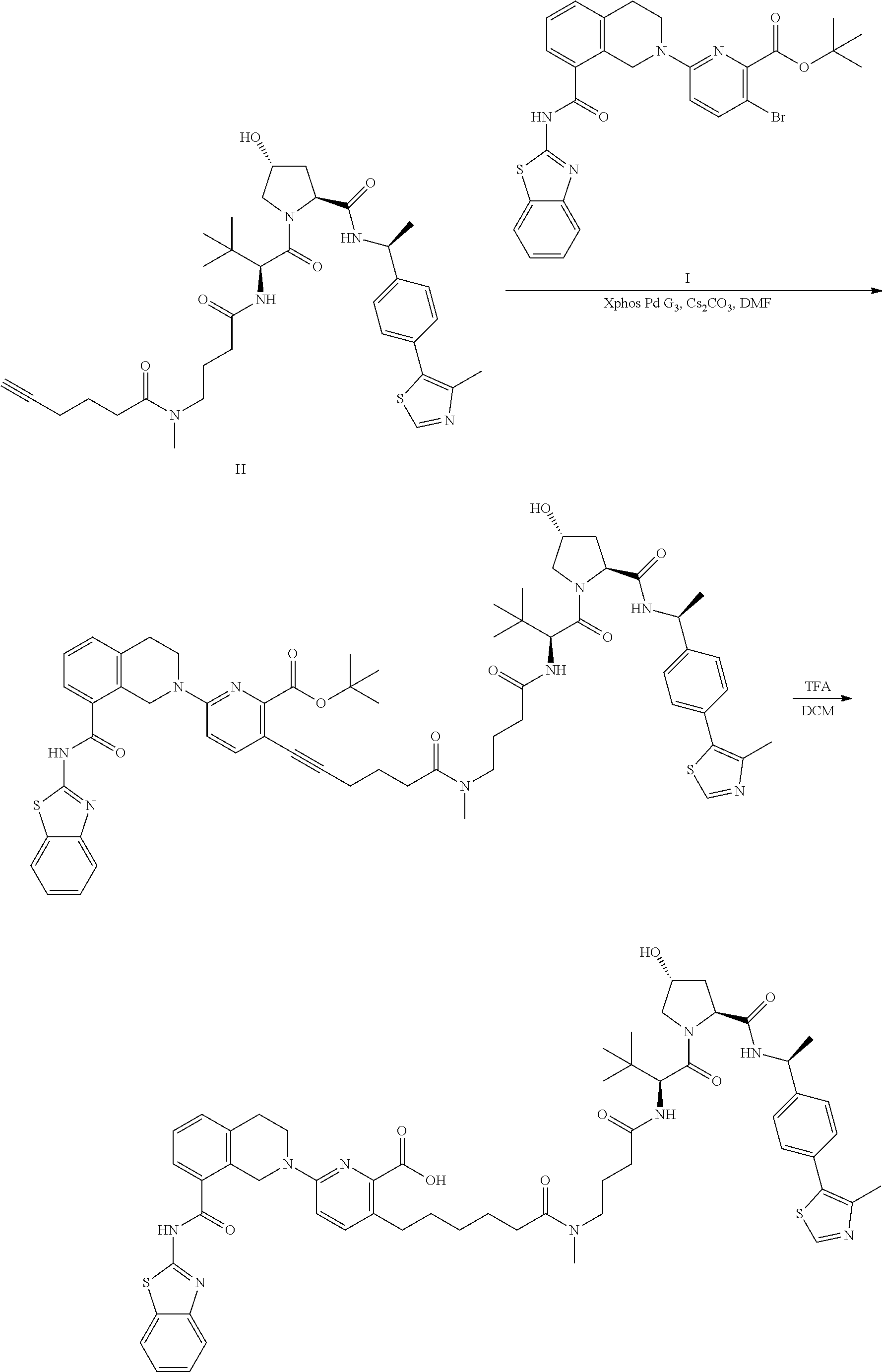

### Step 1—tert-butyl 6-(8-(benzo[d]thiazol-2-ylcarbamoyl)-3,4-dihydroisoquinolin-2 (1H)-yl)-3-(6-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)amino)-6-oxohex-1-yn-1-yl)picolinate

The mixture of tert-butyl 6-(8-(benzo[d]thiazol-2-ylcarbamoyl)-3,4-dihydroisoquinolin-2 (1H)-yl)-3-bromopicolinate (150 mg. 0.265 mmol, Intermediate I), (2S,4R)-1-((S)-3,3-dimethyl-2-(4-(N-methylhex-5-ynamido)butanamido)butanoyl)-4-hydroxy-N-((S)-1-(4-(4-methylthiazol-5-yl)phenyl)ethyl)pyrrolidine-2-carboxamide (169 mg, 0.265 mmol, Intermediate H), XPhos Pd G3 (22.4 mg, 0.0270 mmol) and Cs₂CO₃ (216 mg. 0.660 mmol) in DMF (3 mL) was degassed and purged with N₂ three times. Then the mixture was stirred at 60° C., for 2 h under N₂ atmosphere. On completion, the reaction mixture was quenched with water (3 ml), and extracted with EtOAc (3 mL×3). The combined organic layers were washed with brine (3 mL×2), dried over Na₂SO₄ and evaporated. The residue was purified by flash silica gel chromatography (ISCOR: 4 g SepaFlash® Silica Flash Column, Eluent of 95% Dichloromethane/Methanol ethergradient @20 mL/min) to the title compound (88.0 mg. 22% yield) as a yellow solid. ¹H NMR (400 MHZ, DMSO-d₆)_{6=8.99} (s, 1H), 8.36 (br d, J=8.0 Hz, 1H), 8.03 (d, J=8.0 Hz, 1H), 7.88 (br t, J=10.0 Hz, 1H), 7.78 (br d, J=8.4 Hz, 1H), 7.62-7.57 (m, 2H), 7.46-7.41 (m, 4H), 7.38 (br d, J=8.0 Hz, 4H), 6.88 (dd, J=3.6, 8.8 Hz, 1H), 5.09 (d, J=3.6 Hz, 1H), 4.98 (s, 1H), 4.92 (br t, J=7.3 Hz, 1H), 4.51 (dd, J=3.6, 9.2 Hz, 1H), 4.42 (br t, J=8.1 Hz, 1H), 4.28 (br s, 1H), 3.82 (br t, J=5.9 Hz, 2H), 3.60 (br s, 1H), 3.57 (s, 3H), 3.27-3.20 (m, 3H), 3.01 (br t, J=5.8 Hz, 2H), 2.46 (s, 3H), 2.42-2.38 (m, 4H), 1.71 (br d, J=4.8 Hz, 2H), 1.37 (br d, J=7.2 Hz, 4H), 1.33 (d, J=3.6 Hz, 9H), 1.24 (s. 3H), 0.93 (br d, J=6.8 Hz, 9H)

### Step 2—6-(8-(benzo[d]thiazol-2-ylcarbamoyl)-3,4-dihydroisoquinolin-2 (1H)-yl)-3-(6-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)amino)-6-oxohexyl)picolinic acid

A mixture of tert-butyl 6-(8-(benzo[d]thiazol-2-ylcarbamoyl)-3,4-dihydroisoquinolin-2 (1H)-yl)-3-(6-((4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)(methyl)amino)-6-oxohex-1-yn-1-yl)picolinate (68.0 mg. 0.0606 mmol) in TFA (0.55 mL) and DCM (2.72 mL) was stirred at 20° C., for 2 h under N₂ atmosphere. On completion, the reaction mixture was concentrated under reduced pressure to give a residue. The residue was purified by reversed-phase HPLC (Waters xbridge 150*25 mm 10 um; mobile phase: [water (NH₄HCO₃)-ACN]: B %: 38%-68%. 8 min) to give the title compound (15.0 mg. 22% yield) as green solid. LC-MS (ESI⁺) m/z 1066.5 (M+H). ¹H NMR (400 MHz. DMSO-d₆) ¿=12.97-12.74 (m, 1H), 9.02-8.89 (m, 1H), 8.38-8.32 (m, 1H), 8.06-8.01 (m, 1H), 7.90-7.83 (m, 1H), 7.79 (br d, J=8.8 Hz, 2H), 7.62 (br d, J=7.2 Hz, 1H), 7.45-7.40 (m, 4H), 7.37 (br d, J=6.8 Hz, 6H), 6.40 (br s, 1H), 5.17-4.98 (m, 4H), 4.94-4.88 (m, 1H), 4.50 (br d, J=10.0 Hz, 1H), 4.44-4.38 (m, 1H), 4.27 (br s, 1H), 3.95 (br s, 2H), 3.60 (br s, 2H), 3.02 (br s, 3H), 2.89 (br s, 2H), 2.76 (br s, 2H), 2.45 (br s, 4H), 2.33 (br s, 2H), 1.85-1.76 (m, 4H), 1.72-1.59 (m, 3H), 1.36 (br d, J=6.0 Hz, 3H), 0.92 (br d, J=6.8 Hz, 9H).

TABLE 6
Compounds synthesized via Method 4, using the corresponding
bromides and alkynes for the coupling.

| | | | LCMS | |
|---|---|---|---|---|
| | | | (ESI+) | |
| | | | m/z | |
| I-#^{a} | Bromide | Alkyne | (M + H)⁺ | ¹H NMR (400 MHz, DMSO-d6) |
| I-37 | I | J | 1123.2 | 12.93-12.75 (m, 1H), 8.98 (s, 1H), 8.35 (br d, J = 8.4 Hz, 1H), |
| | | | | 8.09-7.99 (m, 1H), 7.93-7.82 (m, 1H), 7.80-7.77 (m, 1H), |
| | | | | 7.65-7.58 (m, 1H), 7.51-7.41 (m, 4H), 7.40-7.29 (m, 6H), |
| | | | | 6.40 (s, 1H), 5.08-5.03 (m, 2H), 4.94-4.86 (m, 1H), 4.56-4.46 |
| | | | | (m, 1H), 4.44-4.37 (m, 1H), 4.32-4.21 (m, 1H), 4.00-3.91 |
| | | | | (m, 2H), 3.60 (br s, 1H), 3.49-3.37 (m, 1H), 3.25-3.19 (m, |
| | | | | 3H), 3.01 (br s, 2H), 2.90 (s, 1H), 2.75 (s, 1H), 2.45 (br s, 3H), |
| | | | | 2.27-2.19 (m, 4H), 2.16-2.09 (m, 2H), 2.04-1.93 (m, 2H), |
| | | | | 1.82-1.71 (m, 2H), 1.66-1.53 (m, 4H), 1.50-1.44 (m, 2H), |
| | | | | 1.37 (br d, J = 7.2 Hz, 2H), 1.31-1.23 (m, 6H), 0.93 (br s, 9H) |

^{a}Step 1 run from 2-16 hrs at 60° C. TEA could also be used as the base. Step 2 was run from 2-20 hrs at rt.

### Example 5 (Method 5); Synthesis of 7-(4-(R)-3-(4-(N-(4-(4-(4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)-N-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-2-azaspiro[3.5]nonane-2-carboxamide (I-125)

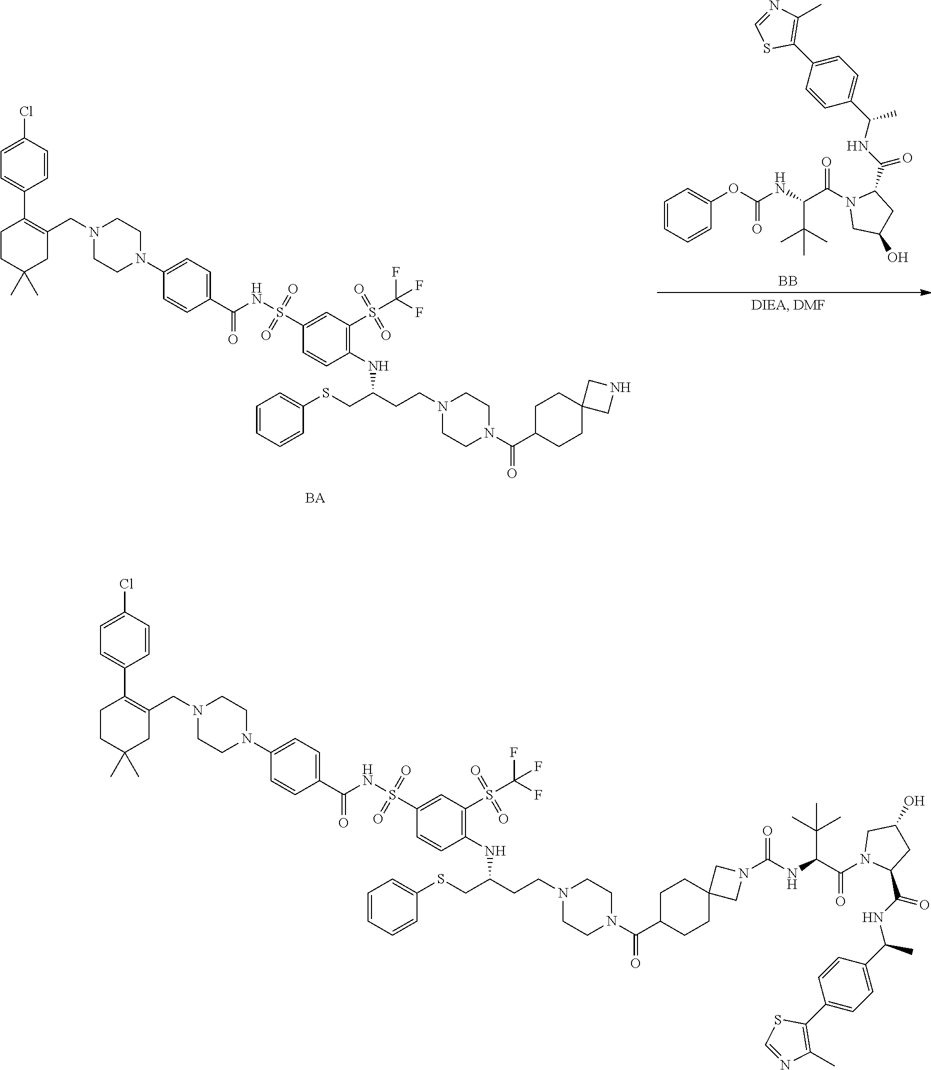 [SMILES: CC1(C)CCC(c2ccc(Cl)cc2)=C(CN2CCN(c3ccc(C(=O)NS(=O)(=O)c4ccc(N[C@H](CCN5CCN(C(=O)C6CCC7(CC6)CNC7)CC5)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)Oc2ccccc2)C(C)(C)C)cc1 ; "BB" ; "DIEA, DMF" ; "BA" ; Cc1ncsc1-c1ccc([C@H](C)NC(=O)[C@@H]2C[C@@H](O)CN2C(=O)[C@@H](NC(=O)N2CC3(CCC(C(=O)N4CCN(CC[C@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)C2)C(C)(C)C)cc1]

To solution of (R)-N-((4-((4-(4-(2-azaspiro[3.5]nonane-7-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1,1′-biphenyl]-2-yl)methyl)piperazin-1-yl)benzamide (55 mg, 49 umol, Intermediate BA) and phenyl N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamate (27.6 mg, 48.9 umol, Intermediate BB) in DMF (1 mL) was added DIEA (31.6 mg, 245 umol), then the mixture was stirred at 80° C., for 2 h. On completion, the mixture was purified by prep-HPLC (neutral condition: column: Phenomenex C18 150*25 mm*10 um; mobile phase: [water (NH₄HCO₃)-ACN]; B %: 53%-63%, 8 min) to give the title compound (19 mg, 24% yield) as a white solid. LC-MS (ESI⁺) m/z 1594.9 (M+H)⁺. ¹H NMR (400 MHZ, DMSO-d₆) δ=8.98 (s, 1H), 8.40-8.33 (m, 1H), 8.12 (s, 1H), 7.98-7.92 (m, 1H), 7.71 (br d, J=8.4 Hz, 2H), 7.45 (br d, J=2.0 Hz, 2H), 7.39-7.35 (m, 4H), 7.34-7.31 (m, 2H), 7.26 (br t, J=7.2 Hz, 2H), 7.20-7.15 (m, 1H), 7.14-7.10 (m, 2H), 6.88-6.82 (m, 2H), 5.55 (br d, J=8.8 Hz, 1H), 5.12-5.06 (m, 1H), 4.94-4.87 (m, 1H), 4.45-4.39 (m, 1H), 4.33-4.26 (m, 2H), 4.11-4.04 (m, 1H), 3.63-3.41 (m, 9H), 3.24-3.15 (m, 5H), 2.89 (s, 1H), 2.73 (s, 1H), 2.69-2.65 (m, 3H), 2.45 (br s, 3H), 2.33 (br s, 4H), 2.27-2.21 (m, 5H), 1.99 (br s, 4H), 1.84-1.74 (m, 4H), 1.60-1.39 (m, 8H), 1.37 (br d, J=7.2 Hz, 3H), 1.34-1.22 (m, 3H), 0.95 (br d, J=17.6 Hz, 14H).

TABLE 7
Compounds synthesized via Method 5, using the corresponding
amines and carbamates for the coupling.

| | | | LCMS | |
|---|---|---|---|---|
| | | | (ESI+) | |
| | | | m/z | |
| I-#^{a} | Amine | Carbamate | (M + H)⁺ | ¹H NMR (400 MHz, DMSO-d6) |
| I-126 | BK | BL | 1625.0 | 8.98 (s, 1H), 8.37 (d, J = 7.2 Hz, 1H), 8.14-8.10 (m, 1H), |
| | | | | 7.95 (br d, J = 9.6 Hz, 1H), 7.71 (br d, J = 8.4 Hz, 2H), 7.45- |
| | | | | 7.42 (m, 2H), 7.37 (d, J = 8.4 Hz, 4H), 7.35-7.32 (m, 2H), |
| | | | | 7.27 (t, J = 7.6 Hz, 2H), 7.20-7.15 (m, 1H), 7.12 (d, J = 8.4 |
| | | | | Hz, 2H), 7.03-6.94 (m, 1H), 6.85 (br d, J = 8.0 Hz, 2H), |
| | | | | 5.69-5.63 (m, 1H), 5.11-5.05 (m, 1H), 4.94-4.87 (m, 1H), |
| | | | | 4.45-4.36 (m, 2H), 4.28 (br s, 1H), 4.11-4.03 (m, 1H), 3.65- |
| | | | | 3.58 (m, 2H), 2.85-2.71 (m, 2H), 2.52 (br d, J = 2.0 Hz, |
| | | | | 9H), 2.45 (s, 3H), 2.31-2.13 (m, 9H), 1.99 (br s, 6H), 1.79 |
| | | | | (ddd, J = 4.4, 8.4, 12.8 Hz, 2H), 1.68-1.62 (m, 2H), 1.53- |
| | | | | 1.31 (m, 14H), 1.24 (br s, 3H), 1.18-1.08 (m, 3H), 1.06 (t, |
| | | | | J = 6.8 Hz, 1H), 0.95 (br d, J = 13.2 Hz, 16H) |
| I-127 | BK | BM | 1551.6 | 8.36 (d, J = 7.6 Hz, 1H), 8.07 (d, J = 1.2 Hz, 1H), 7.92 (dd, |
| | | | | J = 2.0, 8.8 Hz, 1H), 7.70 (d, J = 8.8 Hz, 2H), 7.42-7.34 (m, |
| | | | | 6H), 7.31-7.27 (m, 4H), 7.22-7.17 (m, 1H), 7.12 (d, J = |
| | | | | 8.4 Hz, 2H), 6.79 (br d, J = 8.8 Hz, 2H), 5.65 (br d, J = 8.8 |
| | | | | Hz, 1H), 5.07 (d, J = 3.6 Hz, 1H), 4.86 (br t, J = 7.2 Hz, 1H), |
| | | | | 4.44-4.34 (m, 2H), 4.26 (br s, 1H), 4.11 (s, 1H), 4.06-3.98 |
| | | | | (m, 1H), 3.65-3.56 (m, 3H), 3.43-3.37 (m, 4H), 3.14 (br s, |
| | | | | 4H), 2.75 (br d, J = 1.2 Hz, 3H), 2.67 (br d, J = 1.6 Hz, 1H), |
| | | | | 2.35-2.19 (m, 12H), 2.02-1.94 (m, 4H), 1.78-1.69 (m, |
| | | | | 2H), 1.69-1.60 (m, 3H), 1.51-1.37 (m, 9H), 1.32 (d, J = |
| | | | | 7.2 Hz, 3H), 1.28-1.20 (m, 6H), 1.17-1.08 (m, 3H), 0.95 |
| | | | | (br d, J = 14.8 Hz, 15H) |

^{a}The reaction was run from 80-90° C. for 2-12 hrs. The final compounds were purified via prep-HPLC and chromatography under a variety of conditions.

### Example 6 (Method 6); Synthesis of 6-(8-(benzo[d]thiazol-2-ylcarbamoyl)-3,4-dihydroisoquinolin-2 (1H)-yl)-3-(1-((1-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)methyl)-5-methyl-1H-pyrazol-4-yl)picolinic acid (I-128)

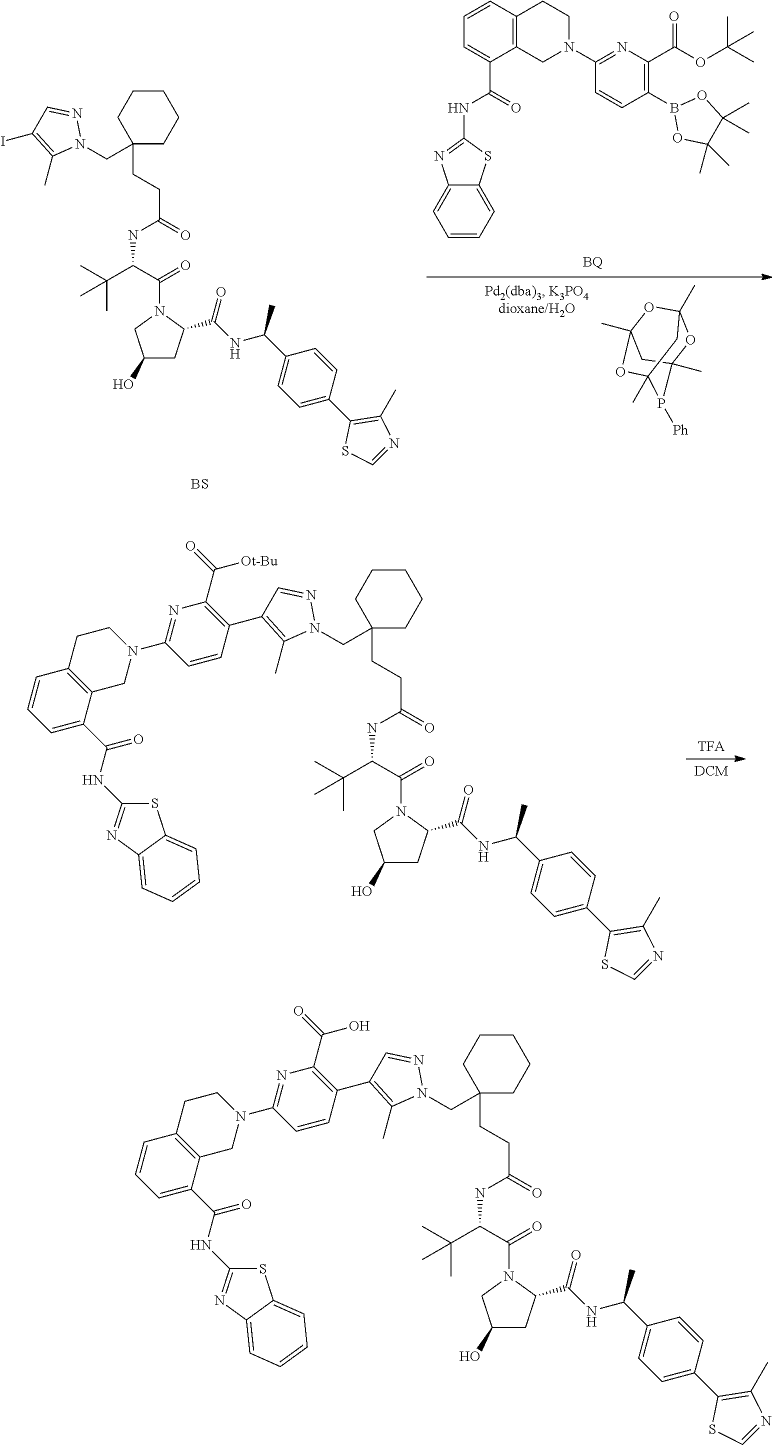

### Step 1—Tert-butyl 6-(8-(benzo]d]thiazol-2-ylcarbamoyl)-3,4-dihydroisoquinolin-2 (1H)-yl)-3-(1-((1-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)methyl)-5-methyl-1H-pyrazol-4-yl)picolinate

To a solution of tert-butyl 6-(8-(benzo[d]thiazol-2-ylcarbamoyl)-3,4-dihydroisoquinolin-2 (1H)-yl)-3-(4,4,5,5-tetramethyl-1,3,2-dioxaborolan-2-yl)picolinate (91.5 mg, 149 umol, Intermediate BQ) and (2S,4R)-4-hydroxy-1-((S)-2-(3-(1-((4-iodo-5-methyl-1H-pyrazol-1-yl)methyl)cyclohexyl)propanamido)-3,3-dimethylbutanoyl)-N-((S)-1-(4-(4-methylthiazol-5-yl)phenyl)ethyl)pyrrolidine-2-carboxamide (80.0 mg, 99.6 umol, Intermediate BS) in dioxane (1 mL) and H₂O (1 mL) was added to Pd₂(dba); (9.13 mg, 9.97 umol), (1S,3R,5R,7S)-1,3,5,7-tetramethyl-8-phenyl-2,4,6-trioxa-8-phosphatricyclo[3.3.1.1^{3,7}]decane (11.6 mg, 39.8 umol, CAS #97739-46-3), and K₃PO₄ (63.4 mg, 298 umol). Then the mixture was stirred at 100° C., for 2 h under N₂. On completion, the reaction mixture was quenched with sat. NH₄Cl (2 mL), and then extracted with EtOAc (3 mL×3). The combined organic layers were washed with brine (3 mL×3), dried over Na₂SO₄, filtered and concentrated under reduced pressure to give a residue. The residue was purified by prep-TLC (SiO₂, Petroleum ether/Ethyl acetate=0/1) to give the title compound (60.0 mg, 26% yield) as a yellow solid. LC-MS (ESI⁺) m/z 1161.5 (M+H)⁺.

### Step 2—6-(8-(benzo[d]thiazol-2-ylcarbamoyl)-3,4-dihydroisoquinolin-2 (1H)-yl)-3-(1-((1-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)methyl)-5-methyl-1H-pyrazol-4-yl)picolinic acid

To a solution of tert-butyl 6-(8-(benzo[d]thiazol-2-ylcarbamoyl)-3,4-dihydroisoquinolin-2 (1H)-yl)-3-(1-((1-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)cyclohexyl)methyl)-5-methyl-1H-pyrazol-4-yl)picolinate (60.0 mg, 51.6 umol) in CH₂Cl₂ (1 mL) was added to TFA (1.54 g, 13.5 mmol), then the mixture was stirred at 25° C., for 12 h. On completion, the reaction mixture was concentrated under reduced pressure to give a residue. The residue was purified by prep-HPLC (column: Phenomenex luna C18 150*25 mm*10 um; mobile phase: [water (FA)-ACN]: B %; 56%-86%, 10 min) to give the title compound (10.5 mg, 18% yield) as a white solid. LC-MS (ESI⁺) m/z 1106.2 (M+H)⁺: ¹H NMR (400 MHZ, DMSO-d₆) δ=8.98 (s, 1H), 8.39 (d, J=7.6 Hz, 1H), 8.05-8.00 (m, 1H), 7.84-7.76 (m, 2H), 7.61 (d, J=6.8 Hz, 1H), 7.50-7.39 (m, 6H), 7.38-7.33 (m, 4H), 6.96-6.86 (m, 1H), 4.97-4.88 (m, 3H), 4.54-4.48 (m, 1H), 4.45-4.39 (m, 1H), 4.31-4.23 (m, 1H), 3.90-3.84 (m, 4H), 3.62-3.56 (m, 2H), 3.02-2.98 (m, 2H), 2.45 (s, 3H), 2.13 (s, 3H), 2.06-1.94 (m, 2H), 1.84-1.71 (m, 2H), 1.62-1.56 (m, 2H), 1.46 (s, 3H), 1.37-1.23 (m, 10H), 1.16-1.13 (m, 1H), 0.94-0.90 (m, 9H).

TABLE 8
Compounds synthesized via Method 6, using the corresponding
iodides and boronic acids for the coupling in Step 1.

| | | | LCMS | |
|---|---|---|---|---|
| | | | (ESI+) | |
| | | Boronic | m/z | |
| I-#^{a} | Iodide | Acid | (M + H)⁺ | ¹H NMR (400 MHz, DMSO-d6) |
| I-129 | CD | BQ | 1190.3 | 12.94-12.76 (m, 2H), 8.98 (s, 1H), 8.37 (d, J = 7.6 Hz, 1H), 8.06- |
| | | | | 8.01 (m, 1H), 7.88-7.82 (m, 1H), 7.81-7.75 (m, 2H), 7.61 (br |
| | | | | d, J = 7.6 Hz, 1H), 7.51-7.47 (m, 2H), 7.46-7.42 (m, 4H), 7.37 |
| | | | | (br d, J = 8.0 Hz, 4H), 7.29 (s, 1H), 6.97-6.93 (m, 1H), 4.95 (br |
| | | | | s, 2H), 4.93-4.88 (m, 1H), 4.50 (d, J = 9.2 Hz, 1H), 4.42 (t, J = |
| | | | | 7.6 Hz, 1H), 4.27 (br d, J = 1.2 Hz, 1H), 3.88 (br t, J = 5.6 Hz, |
| | | | | 3H), 3.85 (s, 2H), 3.59 (br d, J = 3.2 Hz, 3H), 3.01 (br s, 6H), 2.45 |
| | | | | (s, 3H), 2.27-2.20 (m, 2H), 2.12 (s, 3H), 2.09-2.05 (m, 2H), 1.59 |
| | | | | (td, J = 3.6, 6.8 Hz, 4H), 1.49-1.44 (m, 4H), 1.36 (br d, J = 7.2 |
| | | | | Hz, 3H), 1.29 (br s, 4H), 0.93 (s, 9H) |
| I-130 | DF | BQ | 1274.3 | 13.00-12.69 (m, 1H), 8.95 (s, 1H), 8.50 (d, J = 8.0 Hz, 1H), 8.03 |
| | | | | (d, J = 7.6 Hz, 1H), 7.84 (d, J = 9.2 Hz, 1H), 7.79 (d, J = 8.0 Hz, |
| | | | | 1H), 7.61 (d, J = 7.6 Hz, 1H), 7.49-7.45 (m, 1H), 7.44-7.39 (m, |
| | | | | 2H), 7.38-7.32 (m, 7H), 7.24-7.21 (m, 1H), 6.76 (d, J = 8.8 Hz, |
| | | | | 1H), 5.21-4.98 (m, 1H), 4.91-4.86 (m, 3H), 4.51 (d, J = 9.2 Hz, |
| | | | | 1H), 4.44 (t, J = 8.0 Hz, 1H), 4.27 (s, 1H), 3.91-3.87 (m, 2H), |
| | | | | 3.85 (s, 2H), 3.65-3.49 (m, 8H), 2.98 (t, J = 5.6 Hz, 2H), 2.76 (s, |
| | | | | 2H), 2.52 (s, 2H), 2.45-2.43 (m, 1H), 2.42 (s, 3H), 2.26-2.19 |
| | | | | (m, 4H), 2.08 (s, 3H), 1.79 (ddd, J = 4.8, 8.4, 12.8 Hz, 1H), 1.73- |
| | | | | 1.67 (m, 2H), 1.58-1.37 (m, 10H), 1.35-1.26 (m, 7H), 0.94 (s, 9H) |
| I-131 | DX | BQ | 1302.4 | 12.92-12.81 (m, 1H), 8.98 (s, 1H), 8.38 (d, J = 8.0 Hz, 1H), 8.03- |
| | | | | (d, J = 7.6 Hz, 1H), 7.82-7.76 (m, 2H), 7.63-7.58 (m, 1H), 7.49- |
| | | | | 7.41 (m, 4H), 7.38-7.33 (m, 5H), 7.24 (s, 1H), 6.82-6.73 (m, |
| | | | | 1H), 4.92-4.87 (m, 3H), 4.51 (d, J = 9.2 Hz, 1H), 4.44-4.39 (m, |
| | | | | 1H), 4.27 (d, J = 3.6 Hz, 1H), 3.90-3.84 (m, 4H), 3.60 (s, 2H), |
| | | | | 2.99 (d, J = 4.4 Hz, 2H), 2.54 (s, 2H), 2.45 (s, 3H), 2.29-2.22 (m, |
| | | | | 5H), 2.16-2.11 (m, 2H), 2.09 (s, 3H), 2.04-1.97 (m, 2H), 1.84- |
| | | | | 1.74 (m, 2H), 1.54-1.34 (m, 20H), 1.30-1.20 (m, 7H), 0.93 (s, 9H) |

^{a}Step 1 was run 2-12 hrs at 100° C. The final compounds were purified via prep-HPLC and chromatography under a variety of conditions

### Example 7: Synthesis of (2S,4R)—N-(2-(3-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl) propoxy)-4 (4-methylthiazol-5-yl)benzyl)-1-((S)-2-(1-fluorocyclopropanecarboxamido)-3,3-dimethylbutanoyl)-4-hydroxypyrrolidine-2-carboxamide (I-132)

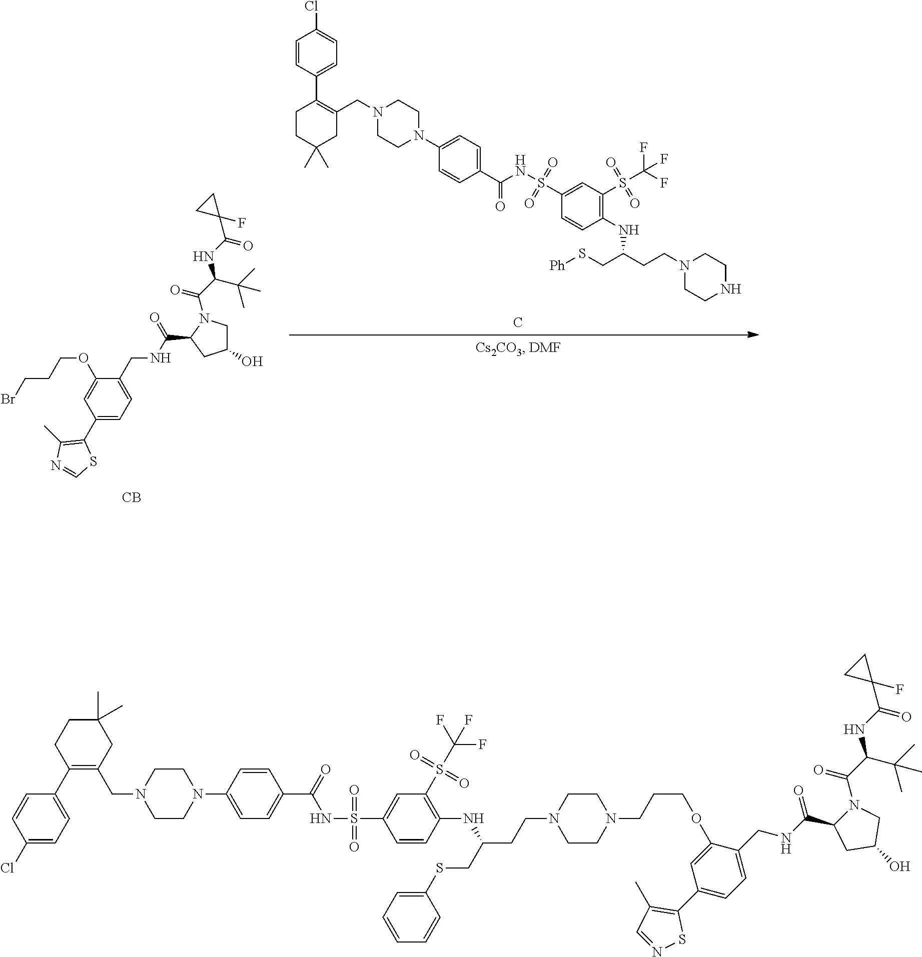

To a solution of (2S,4R)—N-(2-(3-bromopropoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (80 mg, 61.2 umol, Intermediate CB) and (R)-4-(4-((4′-chloro-4,4-dimethyl-3,4,5,6-tetrahydro-[1, l′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (59.6 mg, 61.2 umol, Intermediate C) in DMF (1 mL) was added Cs₂CO; (39.9 mg, 122 umol). The mixture was stirred at 90° C., for 12 h. On completion, the reaction mixture was filtered and concentrated under reduced pressure to give a residue. The residue was purified by prep-HPLC (column: Phenomenex luna C18 150*25 mm*10 um; mobile phase: [water (FA)-ACN]; B %: 33%-63%, 10 min) to give the title compound (28 mg, 28% yield, 95% purity) as a yellow solid. ¹H NMR (400 MHZ, DMSO-d₆) δ 8.99-8.96 (m, 1H), 8.53-8.47 (m, 1H), 8.07 (s, 1H), 7.95 (br d, J=8.0 Hz, 1H), 7.70 (br d, J=8.6 Hz, 2H), 7.42 (br d, J=7.6 Hz, 1H), 7.36 (br t, J=8.4 Hz, 4H), 7.32-7.23 (m, 4H), 7.19 (br t, J=7.2 Hz, 1H), 7.11 (d, J=8.4 Hz, 2H), 7.00-6.95 (m, 2H), 6.92-6.85 (m, 1H), 6.81-6.68 (m, 3H), 5.17 (br d, J=3.4 Hz, 1H), 4.63-4.56 (m, 1H), 4.52 (br t, J=8.0 Hz, 1H), 4.38-4.17 (m, 4H), 4.13-3.97 (m, 4H), 3.67-3.57 (m, 2H), 3.17-3.08 (m, 5H), 2.74 (br s, 2H), 2.46-2.44 (m, 8H), 2.29-2.19 (m. 8H), 2.13-2.04 (m. 3H), 2.02-1.88 (m. 7H), 1.45-1.30 (m. 5H), 1.22 (br dd, J=2.8, 8.6 Hz. 2H), 0.95 (br d, J=3.2 Hz, 15H); LC-MS (ESI) m/z 1548.1 (M+H).

### Example 8. BCL-XL Degradation Assay

Compounds treatment: compounds were reconstituted in DMSO to make a stock concentration of 10 mM. MOLT-4 cells were maintained in RPMI-1640+10% FBS+1% Penicillin-Streptomycin. Cells were seeded into 6-well plates in 2 mL at the seeding density of 2e6 cells/mL. Compounds were added to the cells to a final concentration ranging 0.02-10 μM. After 16 hr incubation at 37° C., in a CO₂ incubator, cells were collected in an Eppendorf tube and centrifuged at 200×g for 5 minutes, the supernatant removed and pellets washed with ice-cold PBS, centrifuged and supernatant removed again. Then. 50 μl of pre-chilled RIPA lysis buffer (Boston BioProducts. BP-115D) with protease and phosphatase inhibitors (Roche Applied Science. #04906837001 and #04693116001) was added into cell pellet to lyze the cells for 20 minutes under 4° C. Cell lysates were spin at 13000 rpm for 10 minutes. Supernatants were collected and protein concentration was measured by BCA assay kit (Solarbio. PC0020-500) following the manufacturer's protocol. Samples' final protein concentration was adjusted to 3.75 μg/uL with the RIPA lysis buffer based on BCA assay results and 4× loading buffer (Invitrogen. NP0007) was added into the samples and samples were heated to 85° C., for 10 min and then let cool at RT. Samples were centrifuged at 13000 rpm for 10 min and then loaded into a 26-well NuPAGER. Novex 4-12% Bis-Tris Midi Gel (Invitrogen. WG1403BOX) and run in NuPAGER MOPS SDS Running Buffer (Invitrogen. NP0001) for 180 minutes at 70 V into a XCell4 SureLock™ Midi-Cell Runner (Invitrogen. WR0100). Gel were transferred using the Invitrogen iBlot gel transfer device (Invitrogen. IB21001) and the nitrocellulose transfer stacks (Invitrogen, IB23001) with Program PO for 9 minutes. The membrane was then blocked with 5% bovin serum albumin for 1 hr at RT, then incubated with primary antibodies diluted in blocking buffer (LI-COR. 927-60001) and shake at 4° C., overnight. The primary antibodies were anti-BCL-xL antibody (CST. 2762:1:1000), anti-beta-actin (CST. 3700; 1:5000) and anti-GAPDH (CST. 97166:1:5000). Membranes were then washed three times with PBST for 15 minutes at RT. Secondary antibodies IR-Dye 680 CW Goat anti Mouse IgG (LI-COR. 926-68070) and IR-Dye 800 CW Goat anti Rabbit IgG (LI-COR. 926-32211) were diluted at a ratio of 1:10000 into PBST and membranes incubated for 1 hour at RT. Membranes were then washed three times with PBST for 15 minutes at RT. The western blot images were obtained using Odyssey Imaging System and quantified using Image Studio Lite Ver 5.2 software.

BCL-XL degradation results in MOLT-4 cells for compounds of the invention are presented in Table 9A and 9B. The letter codes for BCL-XL DC50 include: A (<0.01 μM); B (0.01-0.1 μM); C (>0.1-0.5 MM); D (>0.5-1.0 μM); and E (>1.0 μM). The letter codes for Dmax % include A (>75%); B (50 to 75%); C (<50%). “-” is inactive or not tested.

TABLE 9A
BCL-XL Degradation Results

| | Bcl-xL WB in MOLT-4 | Bcl-xL WB in MOLT-4 |
|---|---|---|
| | degradation 16 h: | degradation 16 h: |
| I-# | Mean external-Abs DC₅₀ | Average Dmax % |
| I-1 | C | A |
| I-2 | C | A |
| I-3 | B | A |
| I-6 | C | A |
| I-7 | D | B |
| I-9 | C | A |
| I-10 | C | A |
| I-11 | C | A |
| I-12 | D | B |
| I-13 | C | A |
| I-14 | C | A |
| I-15 | D | B |
| I-16 | C | A |
| I-17 | B | A |
| I-18 | E | B |
| I-19 | E | C |
| I-20 | C | A |
| I-26 | A | A |
| I-27 | A | A |
| I-30 | E | C |
| I-36 | E | C |
| I-37 | E | C |
| I-38 | C | B |
| I-39 | B | A |
| I-40 | E | C |
| I-43 | C | B |
| I-44 | C | A |
| I-45 | B | A |
| I-46 | C | B |
| I-47 | E | C |
| I-48 | C | B |
| I-72 | — | C |
| I-86 | E | B |
| I-87 | B | A |
| I-88 | B | A |
| I-89 | E | C |
| I-90 | E | C |
| I-91 | E | C |
| I-92 | C | A |
| I-93 | E | B |
| I-95 | A | A |
| I-96 | B | A |
| I-97 | E | C |
| I-98 | E | C |
| I-99 | E | B |
| I-100 | C | B |
| I-101 | E | C |
| I-102 | E | B |
| I-103 | E | C |
| I-104 | E | C |
| I-105 | C | B |
| I-106 | E | C |
| I-107 | E | B |
| I-108 | B | A |
| I-109 | E | C |
| I-110 | E | C |
| I-112 | D | B |
| I-113 | E | C |
| I-114 | E | C |
| I-115 | E | C |
| I-116 | B | A |
| I-117 | A | A |
| I-118 | A | A |
| I-119 | E | C |
| I-120 | B | A |
| I-121 | A | A |
| I-122 | A | A |
| I-123 | A | A |
| I-124 | E | C |
| I-125 | E | C |
| I-126 | E | C |
| I-127 | E | C |
| I-128 | E | C |
| I-129 | C | A |
| I-130 | C | B |
| I-131 | C | B |
| I-132 | B | A |
| I-133 | B | A |
| I-134 | E | C |
| I-135 | B | A |
| I-136 | E | C |
| I-137 | C | A |
| I-138 | E | C |
| I-139 | C | A |
| I-140 | A | A |
| I-141 | A | A |
| I-142 | C | A |
| I-143 | A | A |
| I-144 | D | B |
| I-145 | C | A |
| I-151 | C | C |
| I-158 | C | B |
| I-170 | B | A |
| I-171 | C | B |
| I-172 | A | A |
| I-173 | E | B |
| I-174 | E | C |
| I-175 | E | C |
| I-176 | B | A |
| I-177 | A | A |
| I-178 | B | A |
| I-179 | B | A |
| I-180 | E | C |
| I-181 | A | A |
| I-182 | B | A |
| I-183 | A | A |
| I-184 | B | A |
| I-185 | B | A |
| I-186 | B | A |
| I-187 | B | A |
| I-188 | E | B |
| I-189 | B | A |
| I-190 | D | B |
| I-191 | C | B |
| I-192 | A | B |
| I-193 | B | A |
| I-194 | A | A |
| I-195 | A | A |
| I-196 | A | B |
| I-197 | B | A |
| I-198 | B | A |
| I-199 | B | A |
| I-200 | C | B |
| I-201 | B | A |
| I-202 | C | B |
| I-203 | E | C |
| I-204 | A | A |
| I-205 | A | A |
| I-206 | A | A |
| I-207 | C | B |
| I-208 | D | B |
| I-209 | B | A |
| I-210 | C | A |
| I-212 | A | A |
| I-213 | C | B |
| I-226 | A | A |
| I-230 | A | A |
| I-234 | B | B |
| I-236 | A | A |
| I-237 | A | A |
| I-241 | A | A |
| I-242 | A | A |
| I-246 | A | A |
| I-248 | B | B |
| I-249 | A | A |
| I-275 | A | A |
| I-340 | C | A |
| I-345 | C | A |
| I-351 | C | B |
| I-353 | B | A |
| I-383 | B | A |
| I-384 | A | A |
| I-385 | B | A |
| I-386 | B | A |
| I-387 | B | A |
| I-388 | B | A |
| I-389 | B | A |
| I-390 | B | A |
| I-391 | B | A |
| I-392 | E | C |
| I-393 | E | C |
| I-394 | A | A |
| I-395 | A | A |
| I-396 | A | A |
| I-397 | A | A |
| I-398 | E | C |
| I-399 | A | A |

TABLE 9B
BCL-XL Degradation Results

| | Bcl-xL ELISA in MOLT-4 | Bcl-xL ELISA in MOLT-4 |
|---|---|---|
| | degradation 6 h: Mean | degradation 6 h: |
| I-# | external-Abs DC₅₀ (nM) | Average Dmax % |
| I-71 | — | C |
| I-72 | B | C |
| I-73 | — | C |
| I-96 | B | B |
| I-118 | A | A |
| I-121 | B | A |
| I-123 | B | A |
| I-158 | E | C |
| I-170 | B | B |
| I-172 | B | B |
| I-176 | C | B |
| I-177 | B | A |
| I-178 | B | B |
| I-179 | B | A |
| I-181 | B | B |
| I-182 | C | B |
| I-183 | C | B |
| I-184 | C | B |
| I-185 | B | B |
| I-186 | B | B |
| I-187 | B | B |
| I-189 | B | B |
| I-192 | E | C |
| I-194 | B | A |
| I-195 | B | A |
| I-197 | E | C |
| I-204 | B | A |
| I-205 | B | A |
| I-206 | B | B |
| I-207 | E | C |
| I-208 | E | C |
| I-209 | C | B |
| I-210 | C | B |
| I-211 | E | C |
| I-212 | C | B |
| I-214 | B | A |
| I-215 | C | B |
| I-216 | E | C |
| I-217 | E | C |
| I-218 | — | C |
| I-219 | B | A |
| I-220 | A | A |
| I-221 | E | C |
| I-222 | — | C |
| I-223 | B | B |
| I-225 | B | B |
| I-226 | B | B |
| I-227 | E | C |
| I-228 | C | B |
| I-229 | — | C |
| I-230 | B | A |
| I-231 | — | C |
| I-232 | C | B |
| I-233 | E | C |
| I-234 | E | C |
| I-235 | — | C |
| I-236 | C | B |
| I-237 | C | B |
| I-238 | — | C |
| I-239 | — | C |
| I-240 | B | A |
| I-241 | B | A |
| I-242 | B | A |
| I-243 | B | A |
| I-244 | B | A |
| I-245 | B | A |
| I-246 | B | A |
| I-247 | E | C |
| I-248 | E | C |
| I-249 | E | C |
| I-250 | B | A |
| I-252 | B | A |
| I-253 | A | A |
| I-254 | A | A |
| I-255 | C | B |
| I-256 | C | B |
| I-258 | C | B |
| I-259 | B | A |
| I-260 | C | B |
| I-261 | B | A |
| I-262 | A | A |
| I-263 | B | B |
| I-264 | B | B |
| I-266 | C | B |
| I-267 | E | C |
| I-268 | A | A |
| I-269 | E | C |
| I-270 | E | C |
| I-271 | B | A |
| I-272 | E | C |
| I-273 | A | A |
| I-274 | B | B |
| I-275 | B | A |
| I-276 | B | A |
| I-278 | B | A |
| I-281 | R | C |
| I-282 | C | B |
| I-283 | A | A |
| I-284 | A | A |
| I-285 | C | B |
| I-286 | B | B |
| I-287 | B | A |
| I-288 | D | B |
| I-289 | B | B |
| I-290 | C | B |
| I-291 | D | C |
| I-292 | C | B |
| I-293 | D | C |
| I-294 | B | B |
| I-295 | B | B |
| I-296 | C | B |
| I-297 | D | C |
| I-298 | D | C |
| I-299 | C | A |
| I-300 | C | B |
| I-301 | E | B |
| I-302 | D | B |
| I-303 | C | A |
| I-304 | A | A |
| I-305 | E | C |
| I-306 | B | A |
| I-307 | E | C |
| I-308 | C | A |
| I-309 | C | B |
| I-310 | B | A |
| I-311 | C | A |
| I-312 | B | A |
| I-313 | E | C |
| I-314 | E | C |
| I-315 | B | A |
| I-316 | C | B |
| I-317 | B | A |
| I-318 | B | A |
| I-319 | E | B |
| I-320 | C | A |
| I-321 | B | A |
| I-322 | C | B |
| I-323 | C | B |
| I-324 | E | C |
| I-325 | C | B |
| I-326 | E | C |
| I-327 | B | A |
| I-328 | B | A |
| I-329 | B | A |
| I-330 | B | B |
| I-331 | C | B |
| I-332 | B | A |
| I-333 | C | B |
| I-334 | C | A |
| I-335 | A | A |
| I-336 | B | A |
| I-337 | A | A |
| I-338 | B | A |
| I-339 | A | A |
| I-340 | B | B |
| I-341 | B | A |
| I-342 | C | A |
| I-343 | B | A |
| I-344 | B | A |
| I-345 | C | B |
| I-346 | B | A |
| I-347 | B | A |
| I-348 | A | A |
| I-349 | A | A |
| I-350 | B | A |
| I-351 | C | A |
| I-352 | A | A |
| I-353 | C | A |
| I-354 | A | A |
| I-355 | C | A |
| I-356 | A | A |
| I-357 | C | A |
| I-358 | B | A |
| I-359 | A | A |
| I-360 | A | A |
| I-361 | B | A |
| I-362 | B | A |
| I-363 | B | A |
| I-364 | A | A |
| I-365 | B | A |
| I-366 | C | A |
| I-367 | B | A |
| I-368 | B | A |
| I-369 | B | A |
| I-370 | B | A |
| I-371 | A | A |
| I-372 | B | A |
| I-373 | B | A |
| I-374 | A | A |
| I-375 | B | A |
| I-376 | B | A |
| I-377 | A | A |
| I-378 | B | A |
| I-379 | A | A |
| I-380 | A | A |
| I-381 | B | A |
| I-382 | A | A |
| I-383 | B | A |
| I-388 | C | B |
| I-389 | E | C |
| I-394 | B | B |
| I-395 | B | C |
| I-396 | D | B |
| I-397 | B | B |
| I-399 | C | C |
| I-400 | B | A |
| I-407 | C | B |
| I-408 | A | B |
| I-409 | B | A |
| I-410 | B | A |
| I-413 | B | A |
| I-414 | E | B |
| I-415 | E | B |
| I-416 | C | A |
| I-417 | E | B |
| I-418 | C | A |
| I-419 | A | A |
| I-420 | B | A |
| I-421 | B | A |
| I-422 | C | A |
| I-423 | A | A |
| I-424 | A | A |
| I-425 | A | A |
| I-426 | A | A |
| I-428 | B | A |
| I-429 | B | A |
| I-430 | B | A |
| I-433 | C | A |
| I-434 | B | A |
| I-436 | D | A |
| I-437 | B | A |
| I-438 | A | A |
| I-440 | A | A |
| I-442 | B | A |
| I-443 | C | A |
| I-444 | A | A |
| I-445 | B | A |
| I-446 | B | A |
| I-447 | B | A |
| I-449 | C | A |
| I-450 | B | A |
| I-451 | B | A |
| I-452 | C | A |
| I-453 | C | A |
| I-454 | B | A |
| I-455 | D | A |
| I-456 | E | C |
| I-457 | D | B |
| I-459 | A | A |
| I-460 | B | A |
| I-463 | B | A |
| I-464 | B | A |
| I-465 | B | A |
| I-466 | C | A |
| I-467 | C | A |
| I-471 | B | A |
| I-473 | D | A |
| I-474 | A | A |
| I-475 | A | A |
| I-476 | A | A |
| I-477 | A | A |
| I-478 | A | A |
| I-479 | A | A |
| I-480 | A | A |
| I-481 | A | A |
| I-482 | A | A |
| I-483 | B | A |
| I-484 | A | A |
| I-486 | B | A |
| I-487 | A | A |
| I-488 | B | A |
| I-489 | B | A |
| I-490 | C | A |
| I-491 | A | A |
| I-492 | B | A |

### Example 9. CTG Cell Viability Assay

Compound-mediated viability effect on MOLT-4 and Caki-2 was quantitatively determined using the CellTiter-Glo® Luminescent Cell Viability Assay kit from Promega (Catalog number G7570) following manufacturer's recommended procedures. Briefly, MOLT-4 and Caki-2 cells were seeded into 384 well plates (Grenier Bio-One, Catalog number 781080) with a density of 10,000 cells per well. Compounds were then added to the assay plate with final top concentration of 10 μM and 1:3 dilution series with total of 9 doses. The final DMSO concentration is normalized to 0.2%. The assay plates were incubated at 37° C., for 3 days under 5% CO₂. Then the assay plate was equilibrated at room temperature for 10 minutes. To determine cell viability, 30 uL CellTiter Glo reagent was added to each well and the assay plate was centrifuged at 1000 rpm for 30 second, incubated at room temperature for 10 min, and analyzed by detecting the luminescence using a multimode plate reader (EnVision 2105, PerkinElmer). The data was then analyzed by software Prism 7.0 from GraphPad and the dose response curves are fit using a three-parameter logistic equation to calculate IC₅₀.

MOLT-4 and Caki-2 cell viability results for compounds of the invention are presented in Table 10. The letter codes for MOLT-4 IC₅₀ include: A (<0.1 μM); B (0.1-1.0 μM); C (>1.0-3.0 μM); and D (>3.0 μM). The letter codes for Caki-2 IC₅₀ include: A (<0.1 μM); B (0.1-1.0 μM); C (>1.0-10.0 μM); and D (>10.0 μM). “-” is inactive or not tested.

TABLE 10
Cell Viability Results

| | CTG Cell Viability Assay - | CTG Chempartner - Caki- |
|---|---|---|
| | MOLT-4 3 day: Average | 2 3 day: Average |
| I-# | external-IC₅₀ (uM) | external-IC₅₀ (uM) |
| I-15 | — | D |
| I-16 | — | D |
| I-17 | — | D |
| I-43 | B | — |
| I-44 | — | D |
| I-45 | C | — |
| I-46 | C | — |
| I-47 | D | — |
| I-48 | B | — |
| I-96 | B | D |
| I-108 | C | — |
| I-116 | A | — |
| I-118 | — | D |
| I-120 | B | D |
| I-121 | A | B |
| I-122 | A | — |
| I-123 | A | C |
| I-133 | C | — |
| I-134 | D | — |
| I-135 | B | — |
| I-136 | D | — |
| I-137 | C | — |
| I-138 | D | — |
| I-139 | C | — |
| I-140 | B | — |
| I-141 | A | C |
| I-142 | D | — |
| I-143 | A | C |
| I-144 | B | — |
| I-145 | D | — |
| I-146 | B | — |
| I-147 | C | — |
| I-148 | A | — |
| I-149 | D | — |
| I-150 | B | — |
| I-151 | C | — |
| I-152 | C | — |
| I-153 | C | — |
| I-154 | C | — |
| I-155 | A | — |
| I-156 | D | — |
| I-157 | D | — |
| I-158 | C | D |
| I-159 | B | — |
| I-160 | D | — |
| I-161 | D | — |
| I-162 | B | — |
| I-163 | B | — |
| I-164 | B | — |
| I-165 | B | — |
| I-166 | B | — |
| I-167 | B | — |
| I-168 | B | D |
| I-169 | B | — |
| I-170 | B | — |
| I-171 | C | — |
| I-172 | B | — |
| I-173 | B | — |
| I-174 | C | — |
| I-175 | B | — |
| I-176 | B | — |
| I-177 | A | C |
| I-178 | B | D |
| I-179 | B | D |
| I-180 | D | — |
| I-181 | B | — |
| I-182 | B | — |
| I-183 | A | D |
| I-184 | B | — |
| I-185 | B | — |
| I-186 | B | — |
| I-187 | B | D |
| I-188 | D | — |
| I-189 | B | — |
| I-190 | B | — |
| I-191 | B | — |
| I-192 | C | — |
| I-193 | B | — |
| I-194 | A | — |
| I-195 | A | C |
| I-196 | B | C |
| I-197 | B | — |
| I-198 | A | D |
| I-199 | B | D |
| I-200 | B | — |
| I-201 | B | D |
| I-202 | C | — |
| I-203 | B | — |
| I-204 | B | — |
| I-205 | B | D |
| I-206 | B | — |
| I-207 | B | — |
| I-208 | B | — |
| I-209 | A | — |
| I-210 | B | — |
| I-211 | B | — |
| I-212 | B | — |
| I-213 | C | — |
| I-214 | B | — |
| I-215 | B | — |
| I-216 | C | — |
| I-217 | C | — |
| I-218 | D | — |
| I-219 | B | — |
| I-220 | A | — |
| I-221 | D | — |
| I-222 | C | — |
| I-283 | A | C |
| I-284 | A | B |
| I-285 | B | C |
| I-286 | B | — |
| I-287 | B | — |
| I-288 | B | — |
| I-289 | B | — |
| I-290 | B | — |
| I-291 | C | C |
| I-292 | B | C |
| I-293 | C | C |
| I-294 | B | C |
| I-295 | B | C |
| I-296 | B | C |
| I-297 | E | C |
| I-298 | B | C |
| I-299 | B | C |
| I-300 | B | C |
| I-301 | B | C |
| I-302 | C | C |
| I-303 | B | C |
| I-304 | B | C |
| I-305 | C | — |
| I-306 | A | C |
| I-307 | C | D |
| I-308 | A | C |
| I-309 | C | C |
| I-310 | A | D |
| I-311 | A | C |
| I-312 | A | C |
| I-313 | C | — |
| I-314 | B | C |
| I-315 | A | D |
| I-316 | B | D |
| I-317 | B | C |
| I-318 | A | C |
| I-319 | D | D |
| I-320 | C | D |
| I-321 | A | C |
| I-322 | B | B |
| I-323 | B | D |
| I-324 | D | D |
| I-325 | A | C |
| I-326 | D | D |
| I-327 | A | C |
| I-328 | A | C |
| I-329 | A | C |
| I-330 | A | C |
| I-331 | B | D |
| I-332 | B | D |
| I-333 | B | D |
| I-334 | C | C |
| I-335 | A | C |
| I-336 | C | D |
| I-337 | A | D |
| I-338 | B | D |
| I-339 | A | C |
| I-340 | B | D |
| I-341 | B | D |
| I-342 | B | D |
| I-343 | A | C |
| I-344 | B | D |
| I-345 | C | D |
| I-346 | B | D |
| I-347 | B | D |
| I-348 | A | D |
| I-349 | A | D |
| I-350 | B | C |
| I-351 | D | D |
| I-352 | A | D |
| I-353 | C | D |
| I-354 | A | D |
| I-355 | C | D |
| I-356 | A | D |
| I-357 | C | C |
| I-358 | A | C |
| I-359 | A | D |
| I-360 | B | D |
| I-361 | A | D |
| I-362 | B | D |
| I-363 | A | D |
| I-364 | A | D |
| I-365 | A | D |
| I-366 | C | D |
| I-367 | A | D |
| I-368 | A | D |
| I-369 | A | D |
| I-370 | A | D |
| I-371 | B | D |
| I-372 | A | D |
| I-373 | B | D |
| I-374 | A | C |
| I-375 | C | D |
| I-376 | D | D |
| I-377 | C | D |
| I-378 | B | D |
| I-379 | A | D |
| I-380 | A | C |
| I-381 | B | D |
| I-382 | A | D |
| I-383 | B | C |
| I-384 | B | C |
| I-387 | — | D |
| I-388 | A | D |
| I-389 | A | C |
| I-392 | D | — |
| I-394 | A | D |
| I-395 | A | B |
| I-396 | B | — |
| I-397 | A | B |
| I-398 | D | — |
| I-399 | B | — |
| I-400 | B | D |
| I-407 | B | C |
| I-408 | A | D |
| I-409 | A | D |
| I-410 | A | D |
| I-411 | B | D |
| I-412 | D | D |
| I-413 | B | D |
| I-414 | D | D |
| I-415 | D | D |
| I-416 | D | D |
| I-417 | D | D |
| I-418 | B | D |
| I-419 | A | C |
| I-420 | A | C |
| I-421 | C | D |
| I-422 | B | D |
| I-423 | A | C |
| I-424 | A | C |
| I-425 | A | C |
| I-426 | A | C |
| I-427 | B | D |
| I-428 | A | D |
| I-429 | B | D |
| I-430 | A | D |
| I-431 | B | D |
| I-432 | C | D |
| I-433 | B | D |
| I-434 | A | D |
| I-435 | A | D |
| I-436 | C | D |
| I-437 | A | D |
| I-438 | A | D |
| I-439 | B | D |
| I-440 | A | D |
| I-441 | C | D |
| I-442 | A | D |
| I-443 | D | D |
| I-444 | A | D |
| I-445 | B | D |
| I-446 | B | D |
| I-447 | B | D |
| I-448 | B | D |
| I-449 | D | D |
| I-450 | C | D |
| I-451 | B | D |
| I-452 | C | D |
| I-453 | D | D |
| I-454 | C | D |
| I-455 | C | D |
| I-456 | D | D |
| I-457 | C | D |
| I-458 | D | D |
| I-459 | A | D |
| I-460 | A | D |
| I-461 | B | D |
| I-462 | B | D |
| I-463 | A | C |
| I-464 | B | D |
| I-465 | B | D |
| I-466 | C | D |
| I-467 | C | D |
| I-468 | C | D |
| I-469 | B | D |
| I-470 | A | D |
| I-471 | A | D |
| I-472 | B | D |
| I-473 | C | D |
| I-474 | A | D |
| I-475 | A | D |
| I-476 | A | D |
| I-477 | A | D |
| I-478 | A | D |
| I-479 | A | D |
| I-480 | A | D |
| I-481 | A | D |
| I-482 | A | D |
| I-483 | A | D |
| I-484 | A | D |
| I-485 | D | D |
| I-486 | B | D |
| I-487 | A | D |
| I-488 | A | D |
| I-489 | A | D |
| I-490 | B | D |
| I-491 | A | D |
| I-492 | B | D |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I-aa-1: 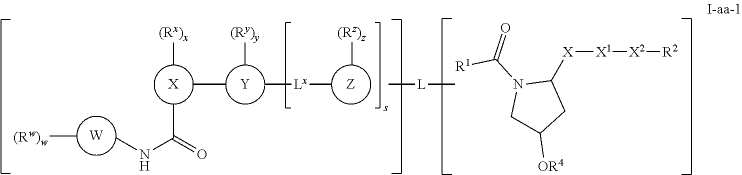
or a pharmaceutically acceptable salt, wherein:
Ring W and Ring Z are, independently, a ring selected from phenyl, naphthyl, a 5-10 membered heteroaryl containing 1-4 heteroatoms independently selected from nitrogen, oxygen, selenium, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring X is a bicyclic ring selected from a 9-11 membered partially unsaturated heterocyclylenyl with 1-4 heteroatoms independently selected from nitrogen, oxygen, and sulfur, and a 9-10 membered heteroarylenyl containing 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R—S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C1-6 aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur:
each R^{A} is independently an optionally substituted group selected from C1-6 aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C1-s hydrocarbon chain, wherein 0-3 methylene units of L^{x} are independently replaced by optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s is 0 or 1; and
w, x, y, and z are, independently, 0, 1, 2, 3, or 4;
L is a covalent bond or a bivalent, saturated or partially unsaturated, straight or branched C1-so hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 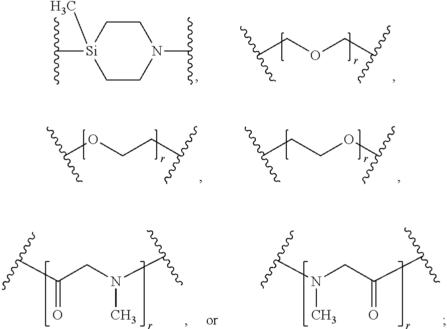
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is-C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C1-6 saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R2 is hydrogen, halogen, —CN 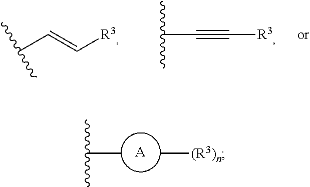
Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R) OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO—R, —NP(O)(R)₂, —N(R)P(O)(OR)₂, —N(R)P(O)(OR)N(R)₂, —N(R)P(O)(N(R)₂)₂, or —N(R)SO₂R; or
two R3 groups are optionally taken together to form an optionally substituted 5-7 membered partially unsaturated or aryl fused ring having 0-2 heteroatoms independently selected from nitrogen, oxygen, and sulfur:
R⁴ is hydrogen, —C(O)R, —C(O)OR, —C(O)NR₂, —P(O)R₂, —P(OXOR)₂, —(CR₂)₁₋₃P(O)R2, —(CR₂)₁₋₃P(O)(OR)₂, —(CR₂)₁₋₃OP(O)R₂, —(CR₂)₁₋₃OP(O)(OR)₂, —C(OX(CR₂)₁₋₃P(O)R₂, —C(O)(CR₂)₁₋₃P(OXOR)₂, —C(OX(CR₂)₁₋₃OP(O)R₂, —C(O)(CR₂)₁₋₃OP(OX(OR)₂, or R^{A};
n is 0, 1, 2, 4, or 5.

2. The compound of claim 1, wherein the compound is a compound of any one of the following formulae: 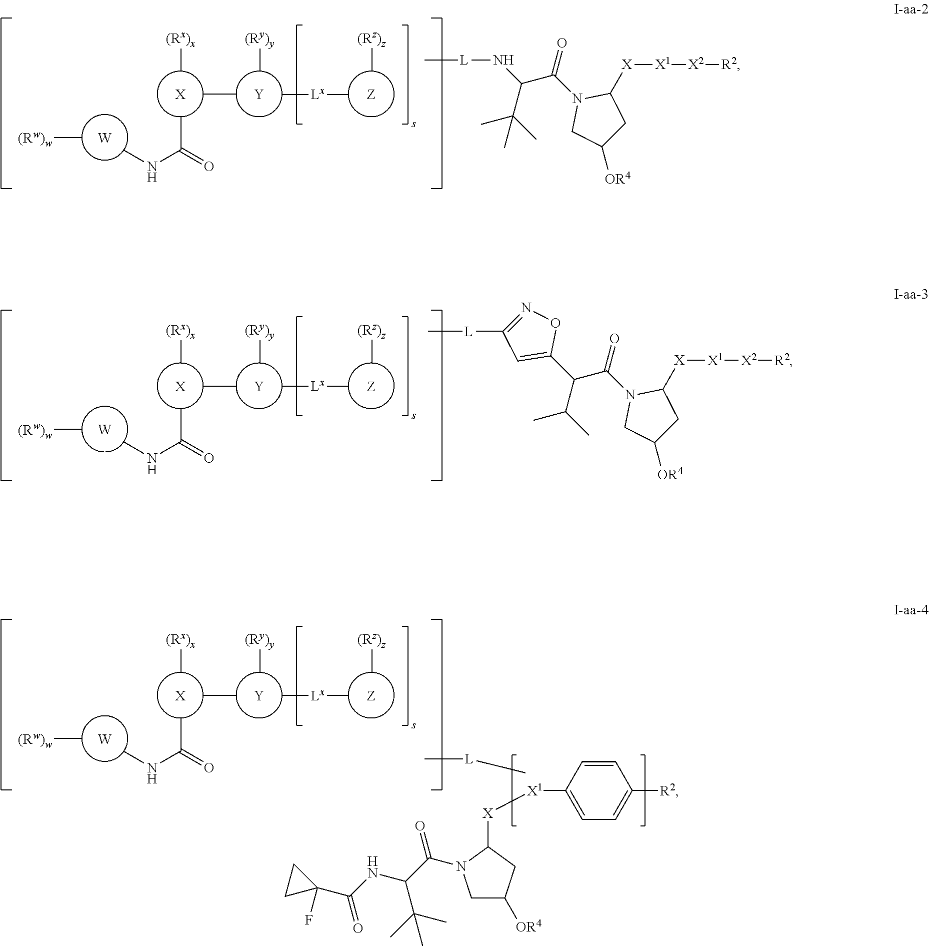 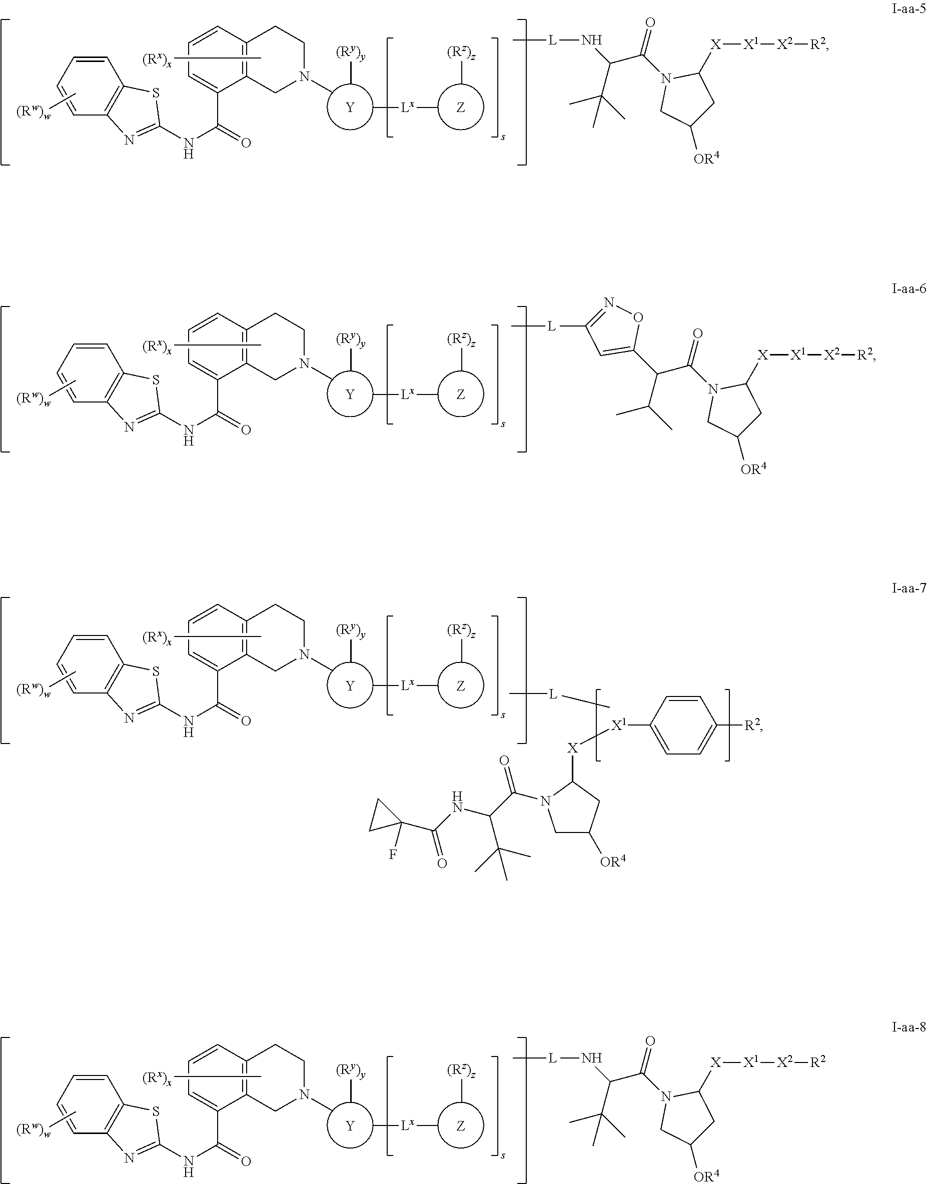
wherein L does not connect (e.g., covalently bond) to BBM through 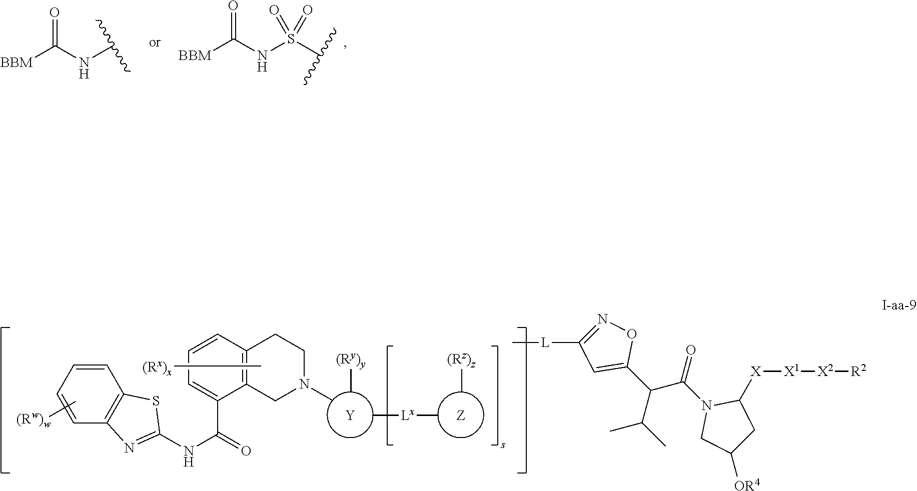
wherein L does not connect (e.g., covalently bond) to BBM through 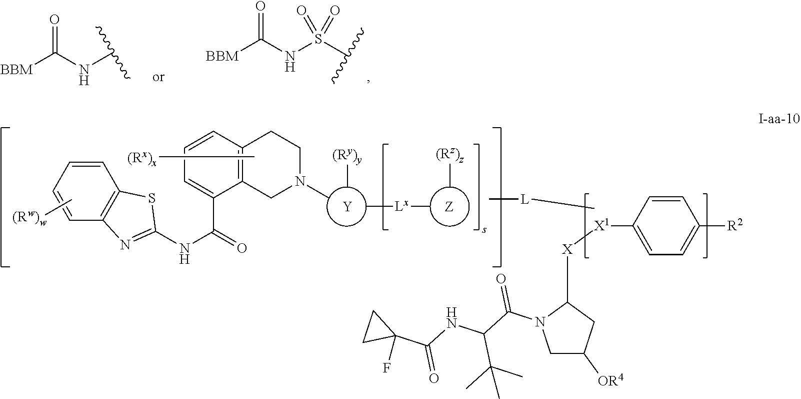
wherein L does not connect (e.g., covalently bond) to BBM through 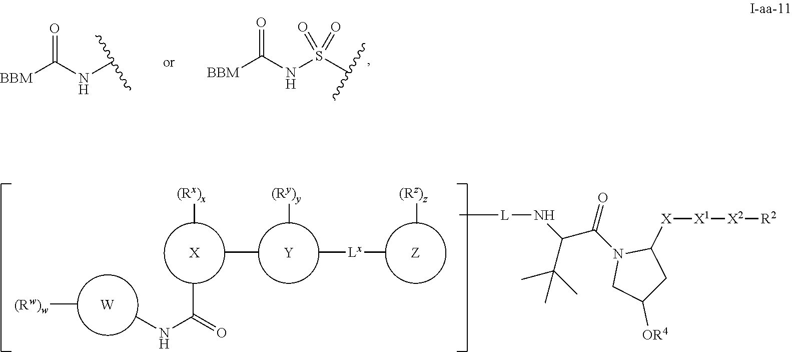
wherein
Ring Z is a ring selected from phenyl, naphthyl, a 5-10 membered heteroaryl containing 1-4 heteroatoms independently selected from nitrogen, oxygen, selenium, and sulfur, and a 5-9 membered saturated or partially unsaturated monocyclic, bicyclic, bridged bicylic, or spirocyclic carbocyclyl or heterocyclyl with 1-4 heteroatoms independently selected from nitrogen, oxygen, and sulfur, 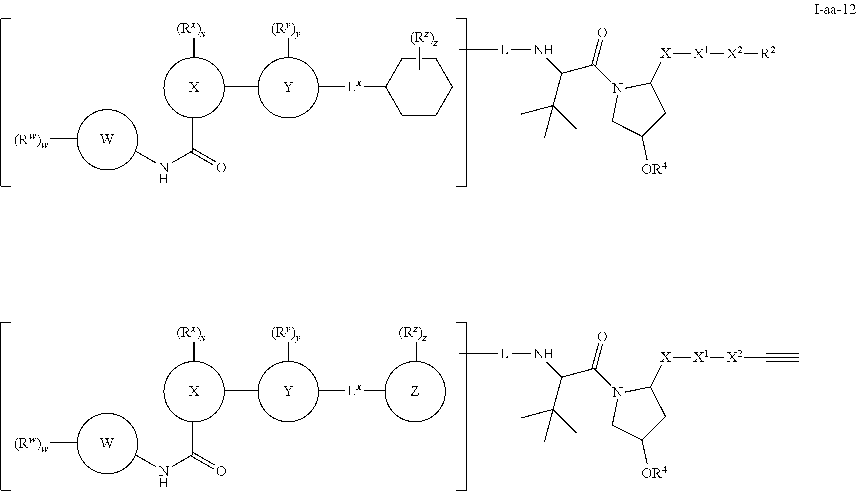
wherein
Ring Z is a ring selected from phenyl, naphthyl, a 5-10 membered heteroaryl containing 1-4 heteroatoms independently selected from nitrogen, oxygen, selenium, and sulfur, and a 5-9 membered saturated or partially unsaturated monocyclic, bicyclic, bridged bicylic, or spirocyclic carbocyclyl or heterocyclyl with 1-4 heteroatoms independently selected from nitrogen, oxygen, and sulfur, 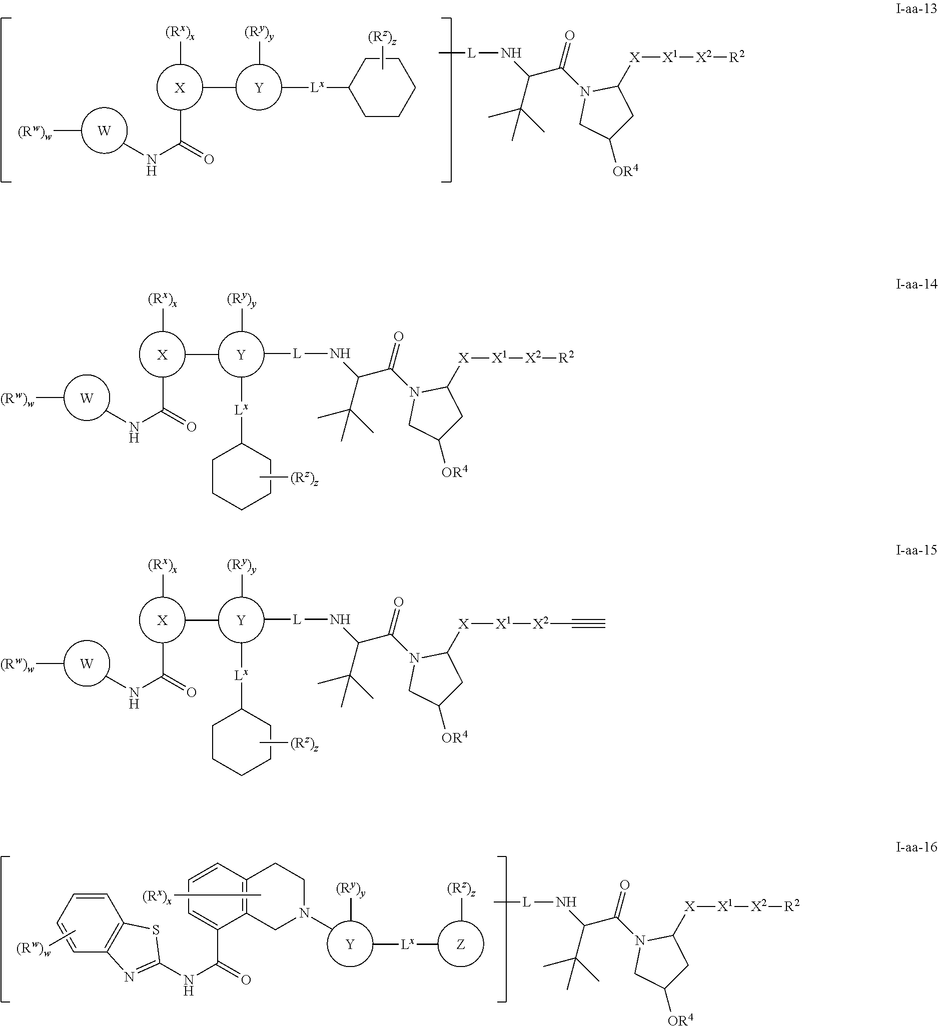
wherein
Ring Z is a ring selected from phenyl, naphthyl, a 5-10 membered heteroaryl containing 1-4 heteroatoms independently selected from nitrogen, oxygen, selenium, and sulfur, and a 5-9 membered saturated or partially unsaturated monocyclic, bicyclic, bridged bicylic, or spirocyclic carbocyclyl or heterocyclyl with 1-4 heteroatoms independently selected from nitrogen, oxygen, and sulfur, 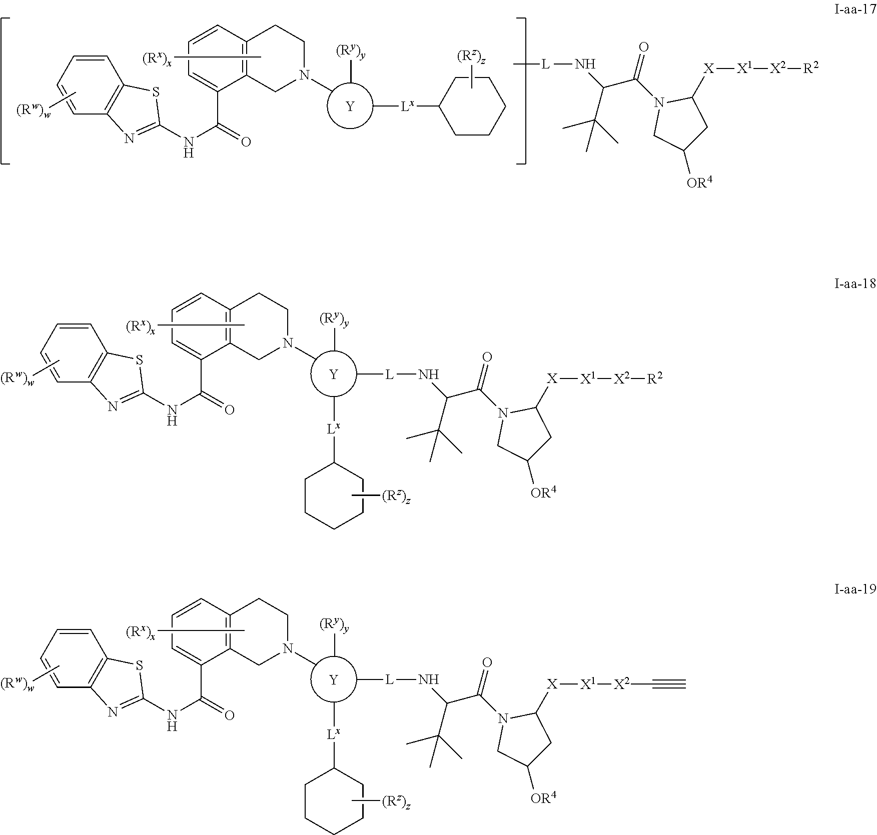
or a pharmaceutically acceptable salt thereof.

3. A compound of formula I-bb-1: 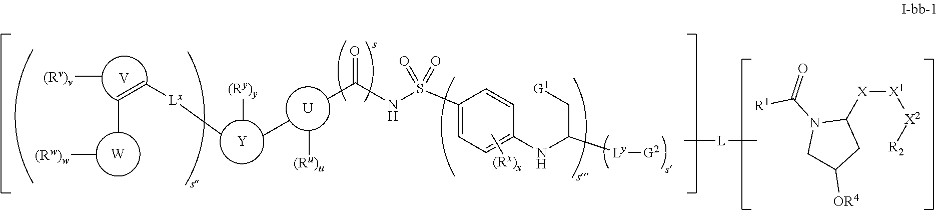
or a pharmaceutically acceptable salt, wherein;
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
Ring V is a bivalent ring selected from a 5-6 membered partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
R^{u}, R^{v}, R^{w}, R^{x1}, R^{y}, and R^{z} are, independently, hydrogen, R^{A}, halogen, —CN, —NO₂, —OR, —SR, —N(R)₂, —Si(R)₃, —S(O)₂R, —S(O)—N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C1-6 aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur:
each R^{A} is independently an optionally substituted group selected from C1-6 aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G¹ is-S-aryl, —S-heteroaryl, or —R^{A};
G² is hydrogen or 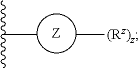
Ring Z is a ring selected from phenyl, naphthyl, a 5-10 membered heteroaryl containing 1-4 heteroatoms independently selected from nitrogen, oxygen, and sulfur, and a 3-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
L^{x} and L^{y} are, independently, a covalent bond or a bivalent, saturated or partially unsaturated, straight or branched C₁₋₅ hydrocarbon chain, wherein 0-3 methylene units of L′ are independently replaced by optionally substituted 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
S, s″, and s′″ are, independently, 0 or 1;
s′ is 1 or 2;
u, v, x, w, y, and z are, independently, 0, 1, 2, 3, or 4;
L is a covalent bond or a bivalent, saturated or partially unsaturated, straight or branched C1-50 hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 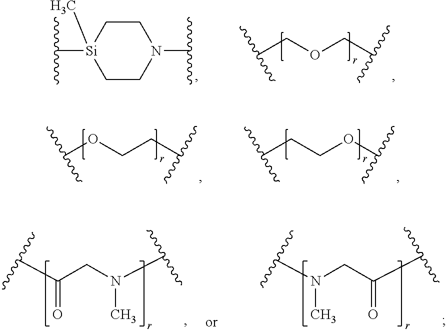
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is-C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-9 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R2 is hydrogen, halogen, —CN, 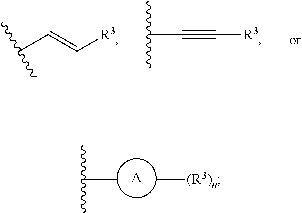
Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R3 is independently hydrogen, R^{A}, halogen, —CN, —NO₂, —OR, —SR, —N(R)₂, —Si(R)₃, —SO₂R, —SON(R)₂, —S(O)R, —C(O)R, —C(O)OR, —C(O)N(R)₂, —C(O)N(R)OR, —C(R)NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R) C(O)OR, —N(R) C(O)R, —NRC(O)N(R)₂, —N(R) SO₂R, —NP(O)(R)₂, —N(R)P(O)(OR)₂, —N(R)P(O)(OR)N(R)₂, —N(R)P(O)(N(R)₂)₂, or —N(R)SO₂R; or
two R3 groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃P(O)R₂, —(CR₂)₁₋₃P(O)(OR)₂, —(CR₂)₁₋₃OP(O)R₂, —(CR₂)₁₋₃OP(O)(OR)₂, —C(O)(CR₂)₁₋₃P(O)R₂, —C(O)(CR₂)₁₋₃P(O)(OR)₂, —C(O)(CR₂)₁₋₃OP(O)R₂, —C(O)(CR₂)₁₋₃OP(O)(OR)₂, or R^{A};
n is 0, 1, 2, 4, or 5.

4. The compound of claim 3, wherein the compound is a compound of any one of the following formulae: 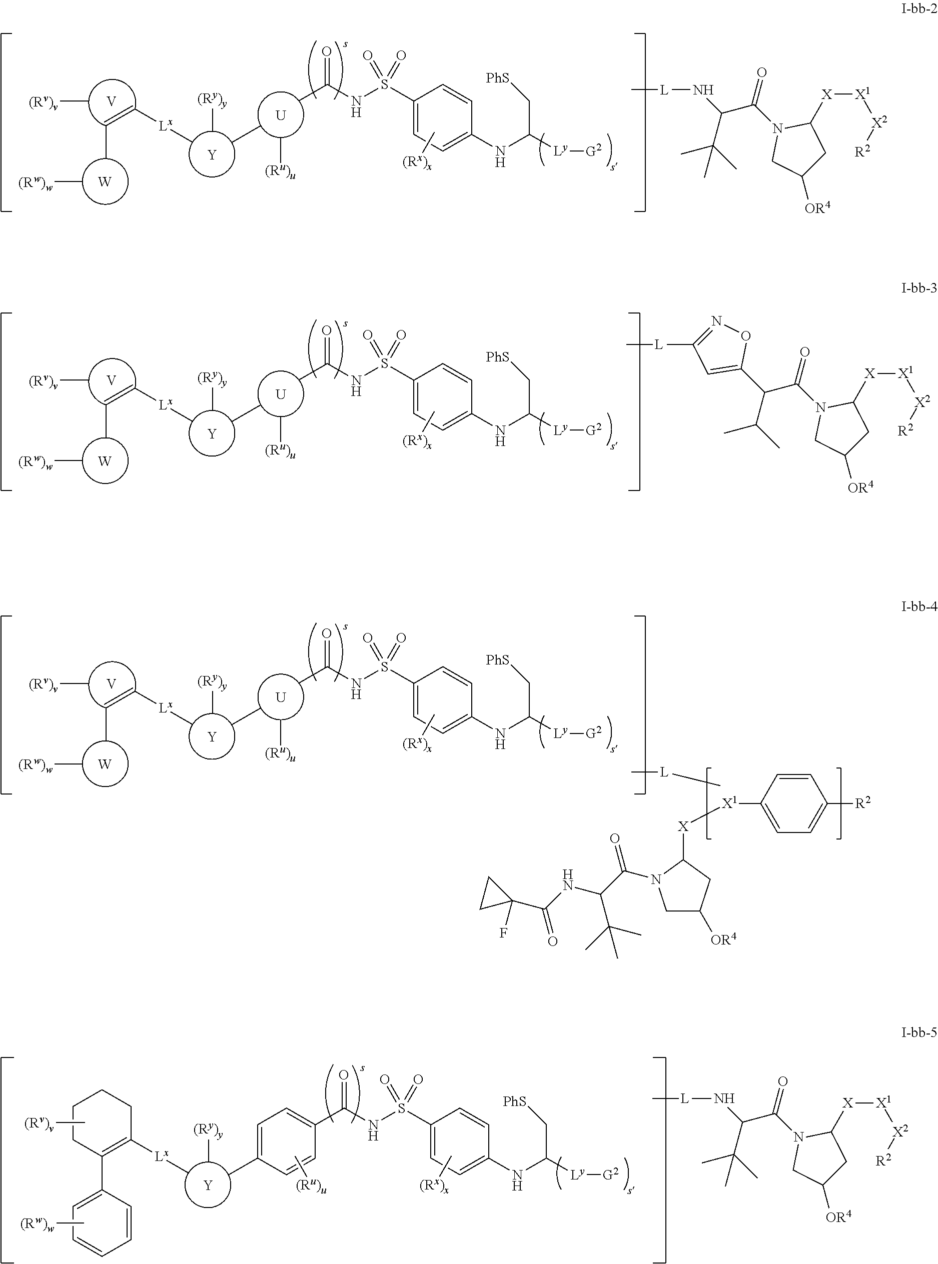 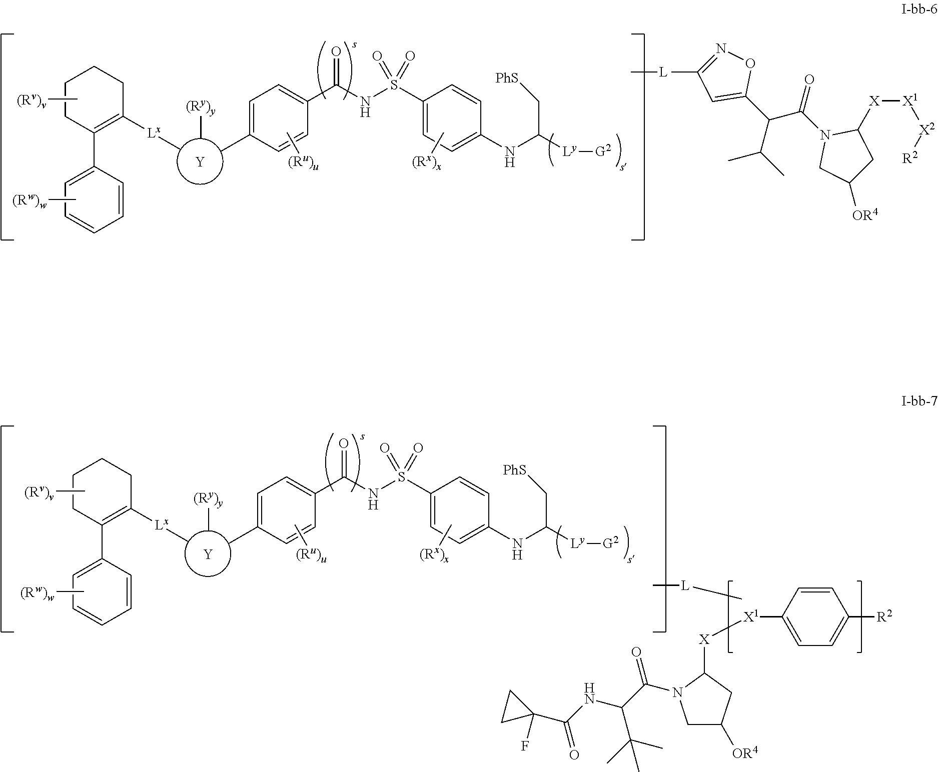
or a pharmaceutically acceptable salt thereof.

5-8. (canceled)

9. The compound of claim 1, wherein Ring W is phenyl or a 5-10 membered heteroaryl containing 1-4 heteroatoms independently selected from nitrogen, oxygen, and sulfur.

10. The compound of claim 1, wherein Ring Y is a 5-7 membered saturated or partially unsaturated heterocyclylenyl with 1-4 heteroatoms independently selected from nitrogen, oxygen, and sulfur, or a 5-6 membered heteroarylenyl containing 1-4 heteroatoms independently selected from nitrogen, oxygen, and sulfur.

11. The compound of claim 1, wherein Ring Z is phenyl, a 5-7 membered saturated or partially unsaturated heterocyclyl with 1-4 heteroatoms independently selected from nitrogen, oxygen, and sulfur, or a 5-6 membered heteroaryl containing 1-4 heteroatoms independently selected from nitrogen, oxygen, and sulfur.

12-13. (canceled)

14. The compound of claim 1, wherein X² is phenylenyl or a 5-6 membered heteroarylenyl containing 1-4 heteroatoms independently selected from nitrogen, oxygen, and sulfur.

15. The compound of claim 1, wherein R2 is

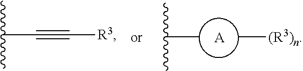

16. The compound of claim 1, wherein Ring A is a 5-6 membered heteroaryl containing 1-4 heteroatoms independently selected from nitrogen, oxygen, and sulfur.

17. The compound of claim 1, wherein L is a covalent bond or a bivalent, saturated or partially unsaturated, straight or branched C₁₋₂₀ hydrocarbon chain, wherein 0-6 methylene units of L are independently replaced by -Cy-, —O—, —N(R)—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, or —N(R)C(O)O—.

18. The compound of claim 1, wherein L is-O—,

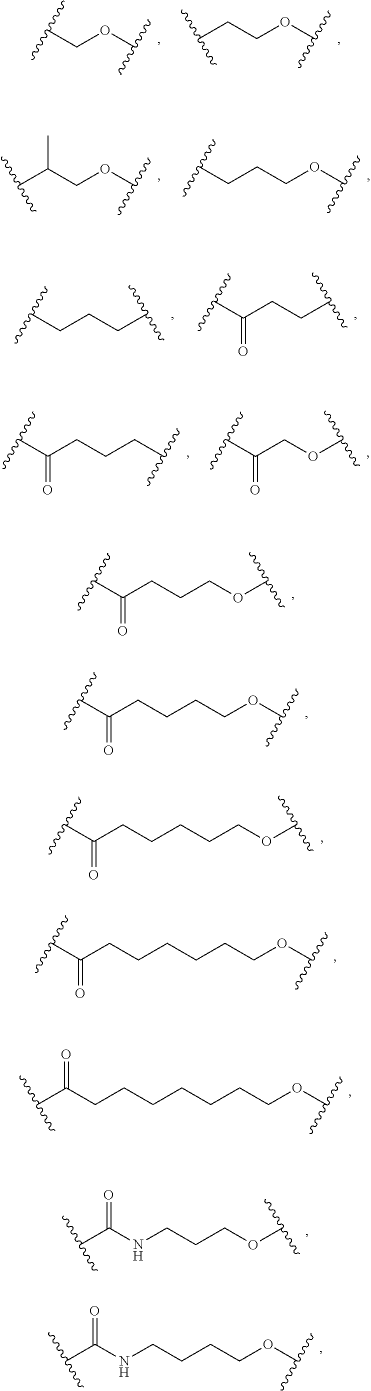

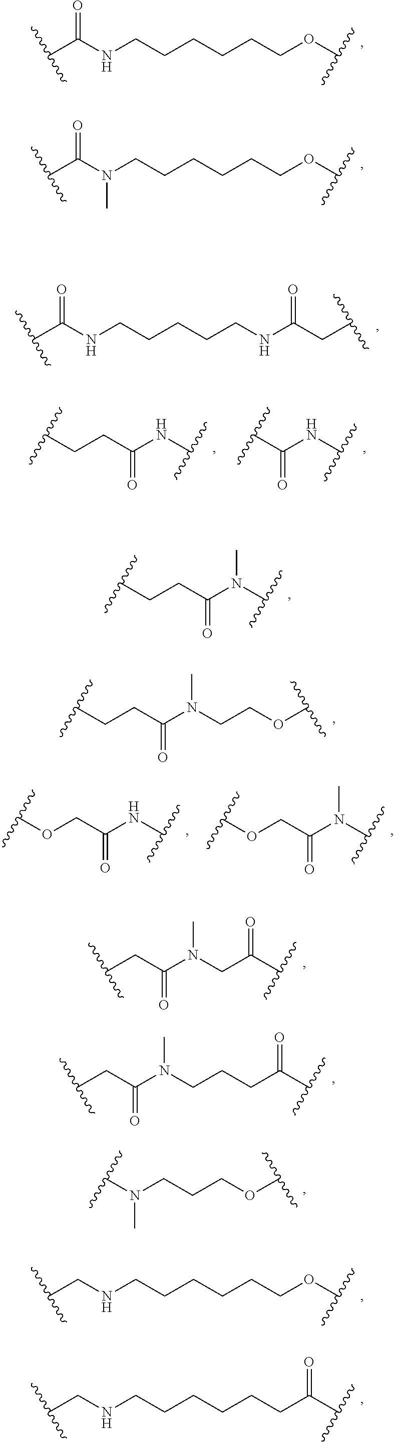

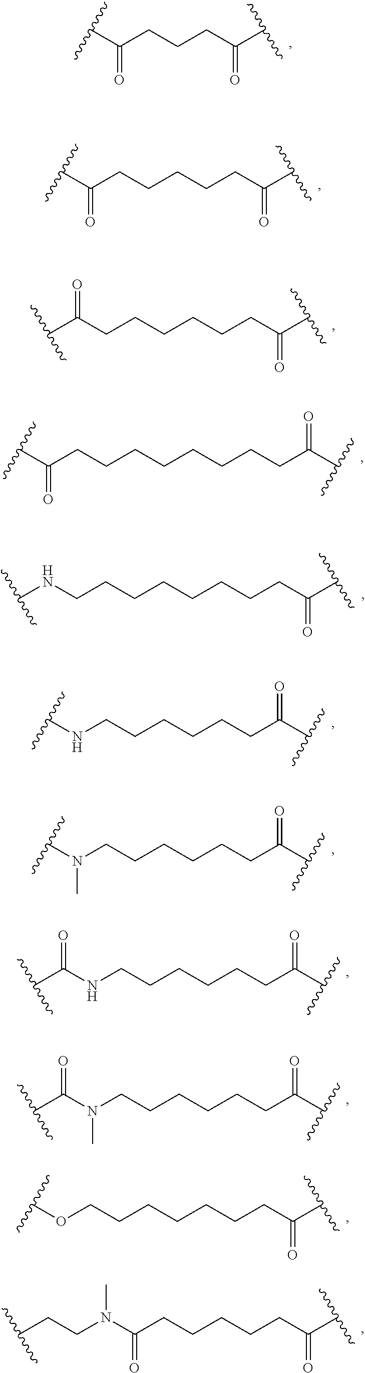

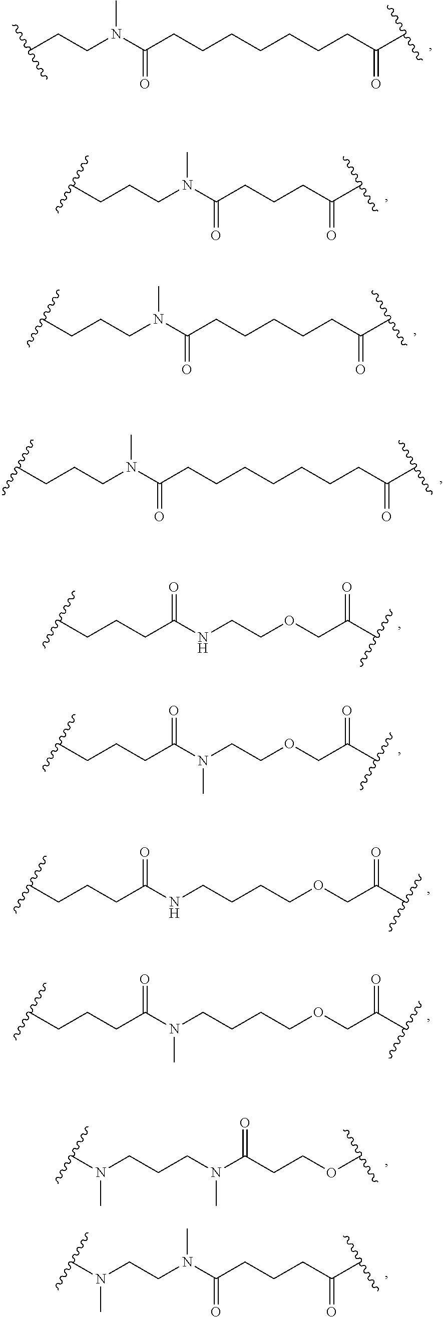

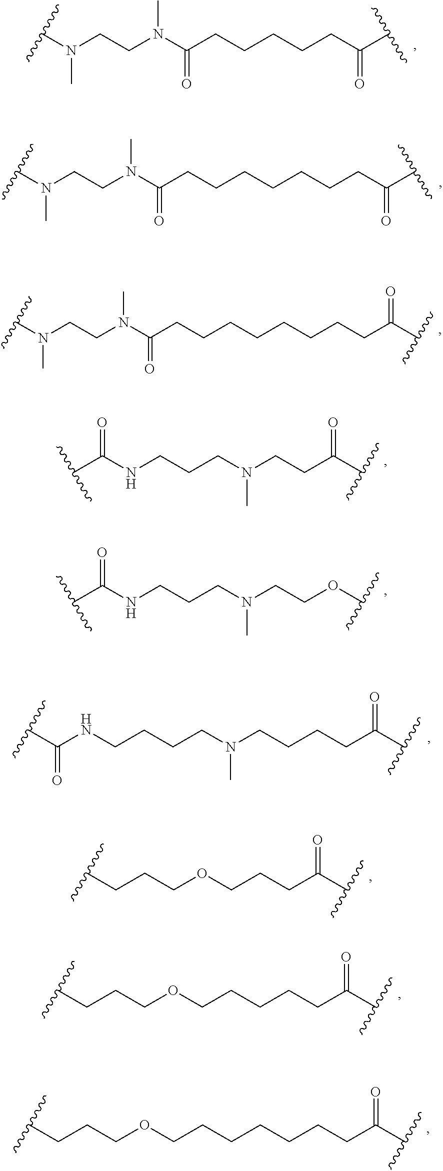

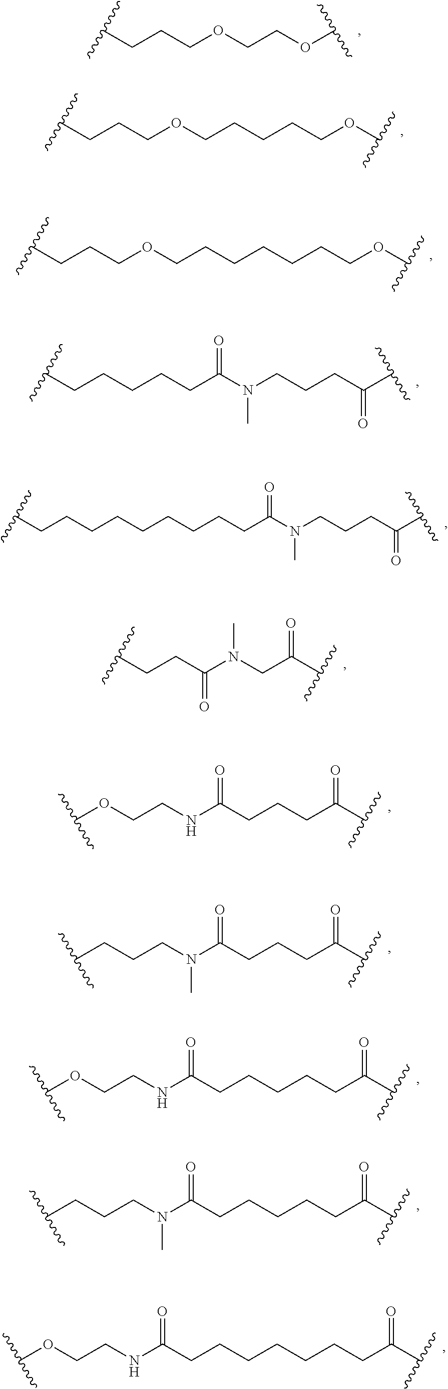

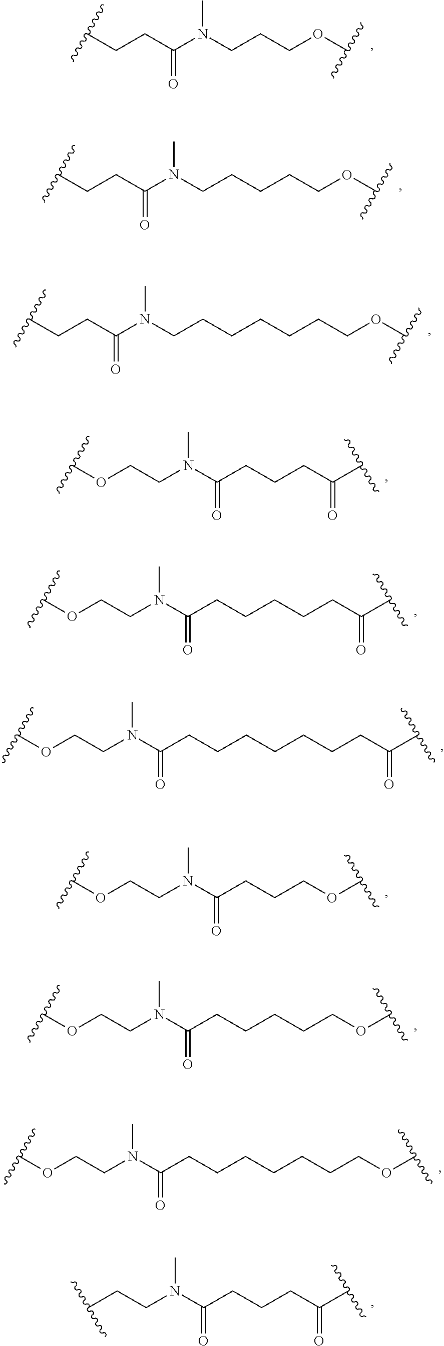

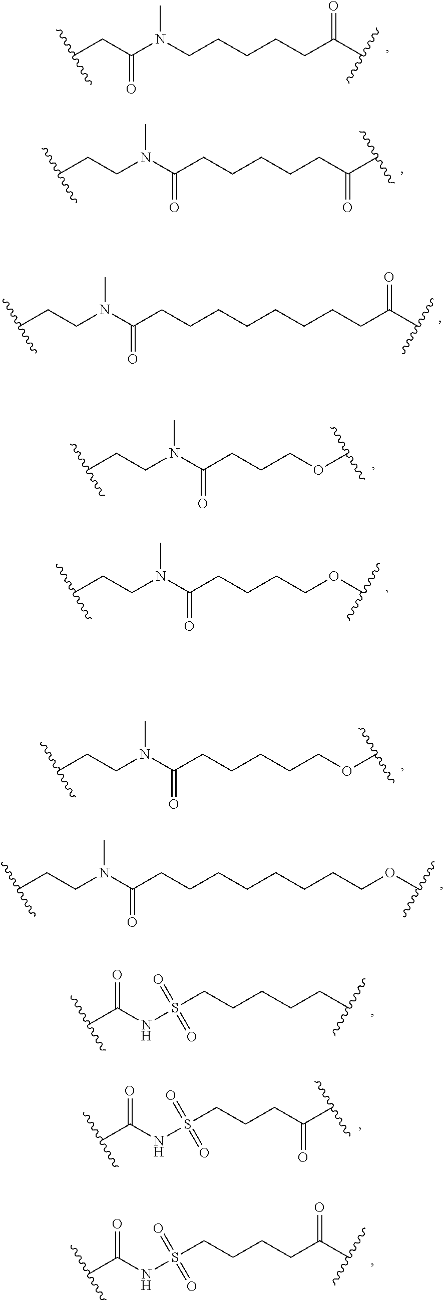

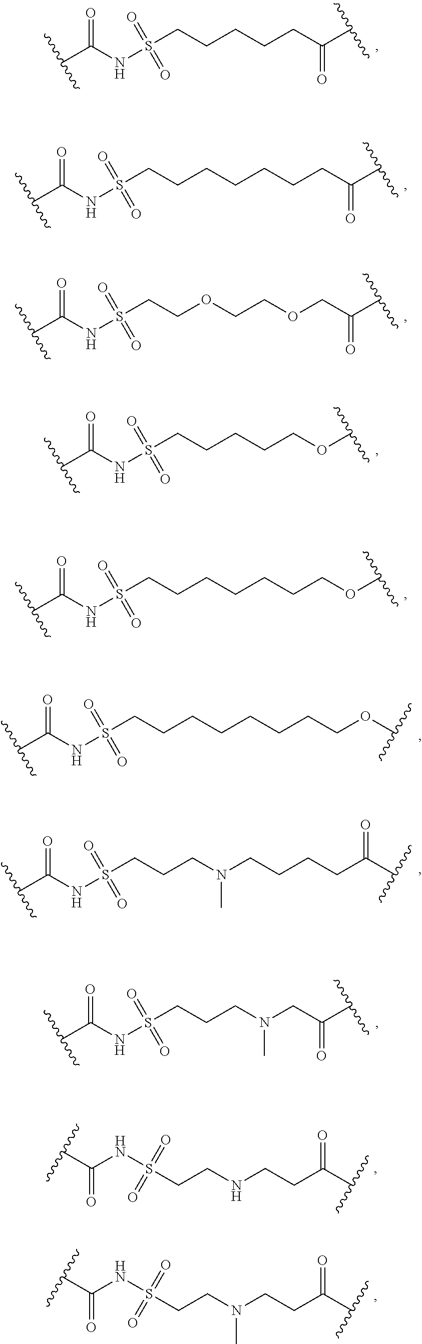

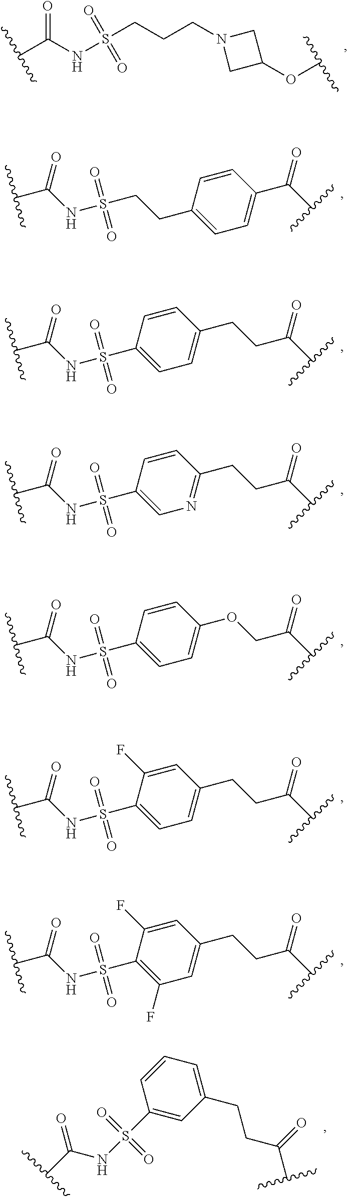

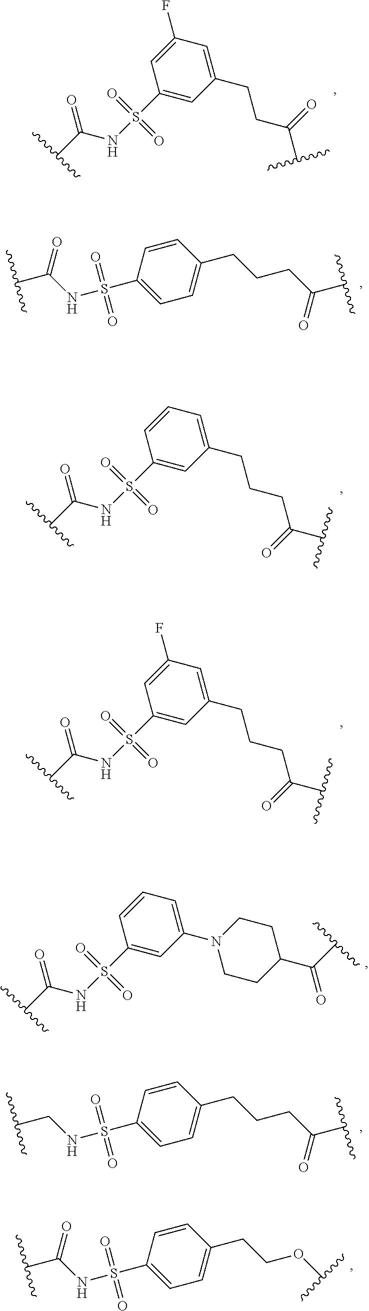

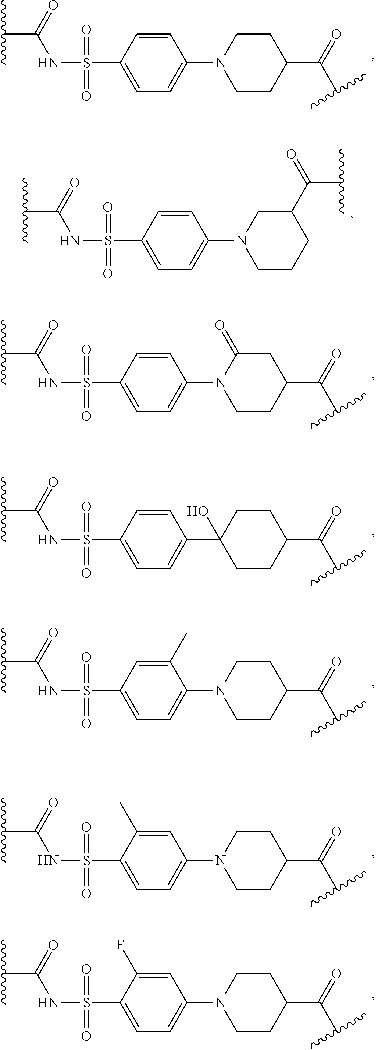

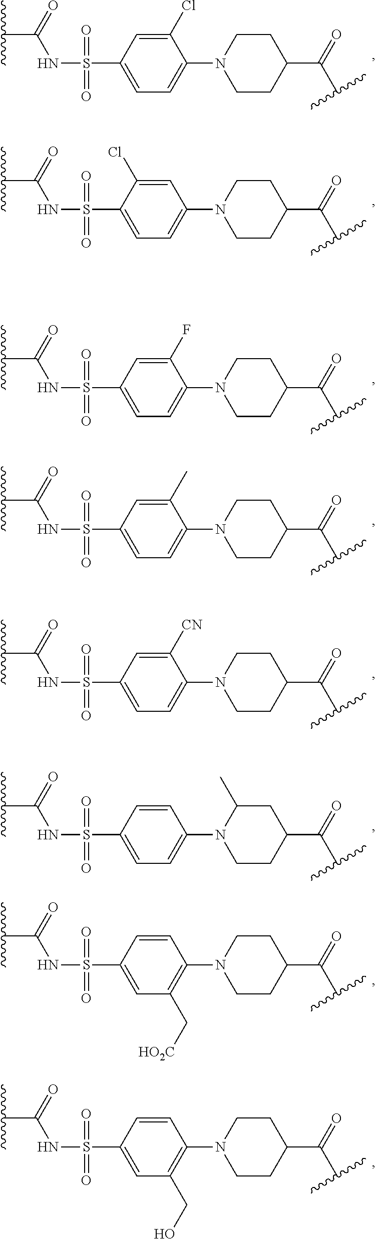

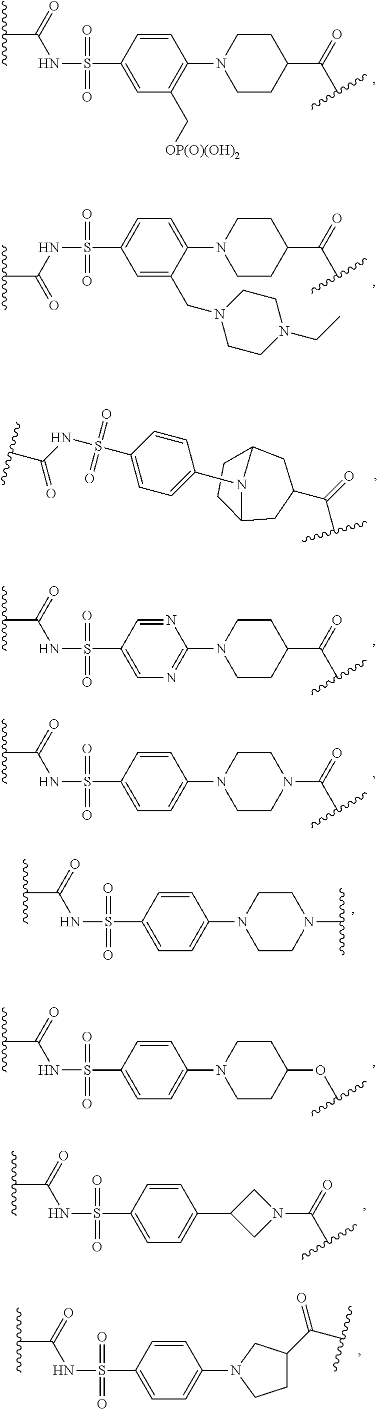

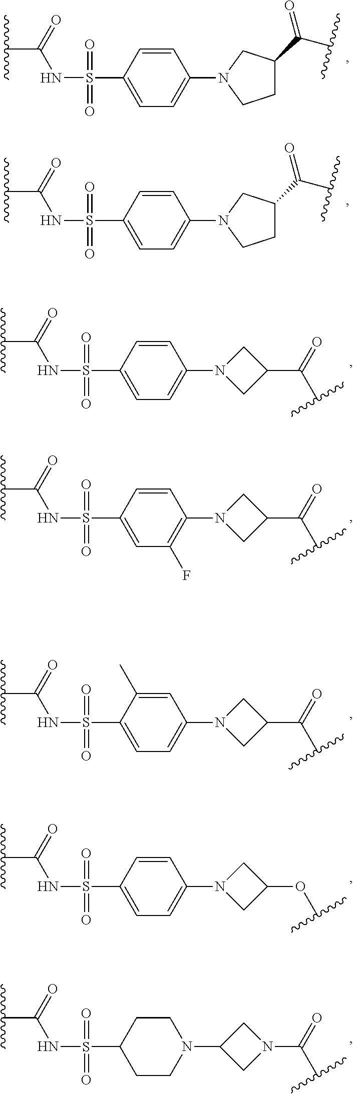

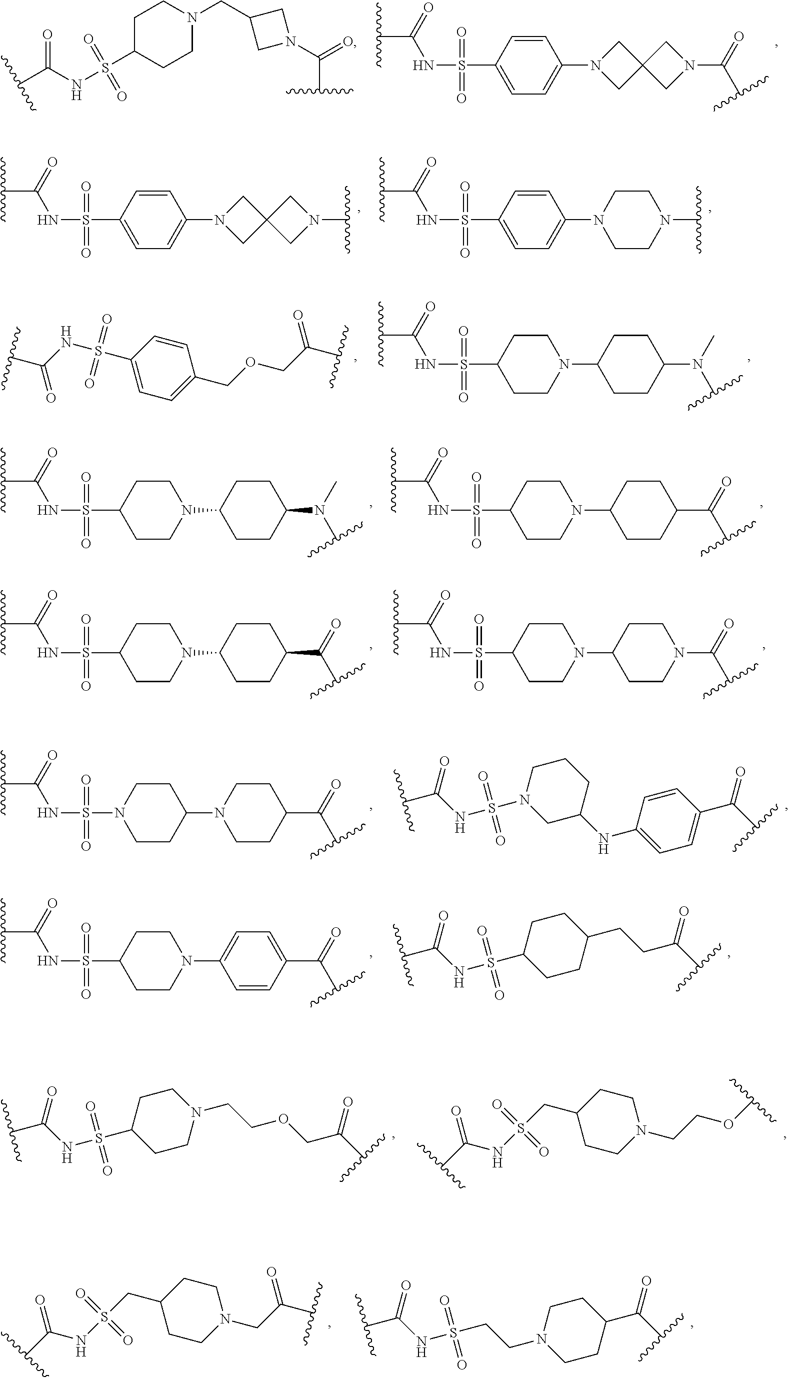

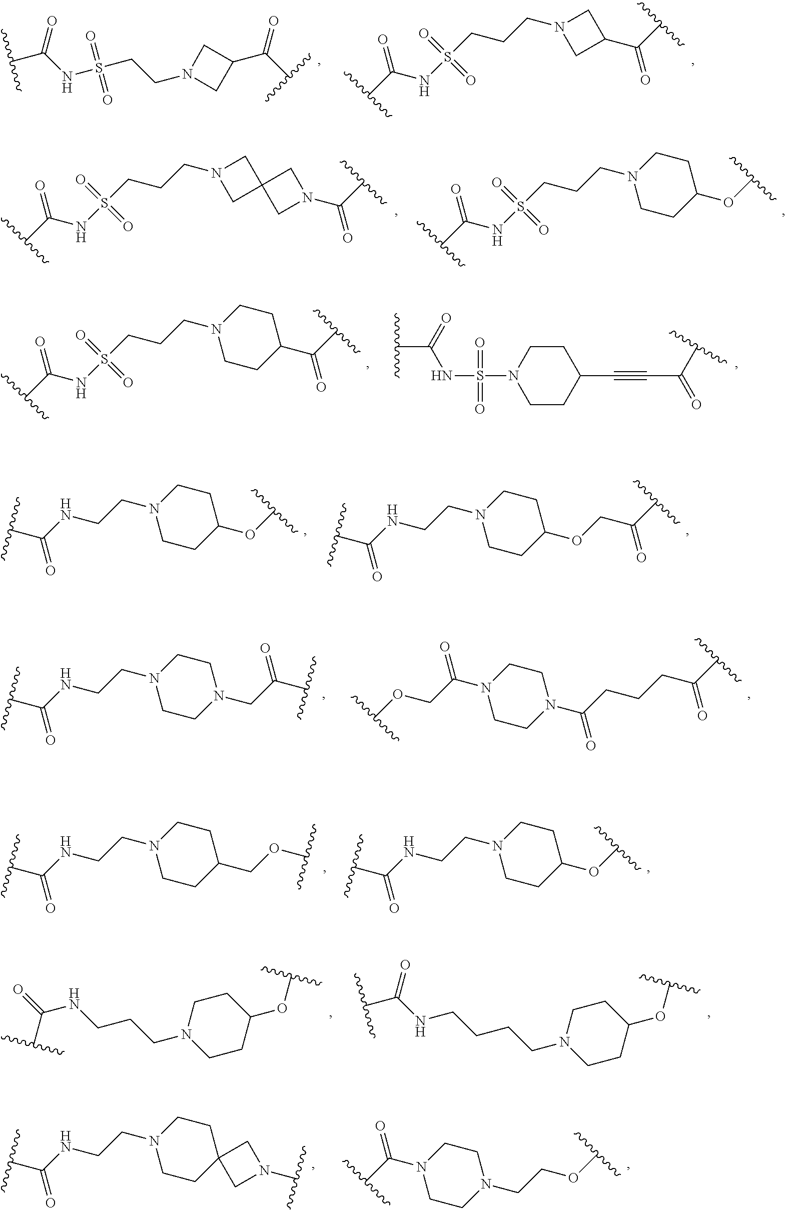

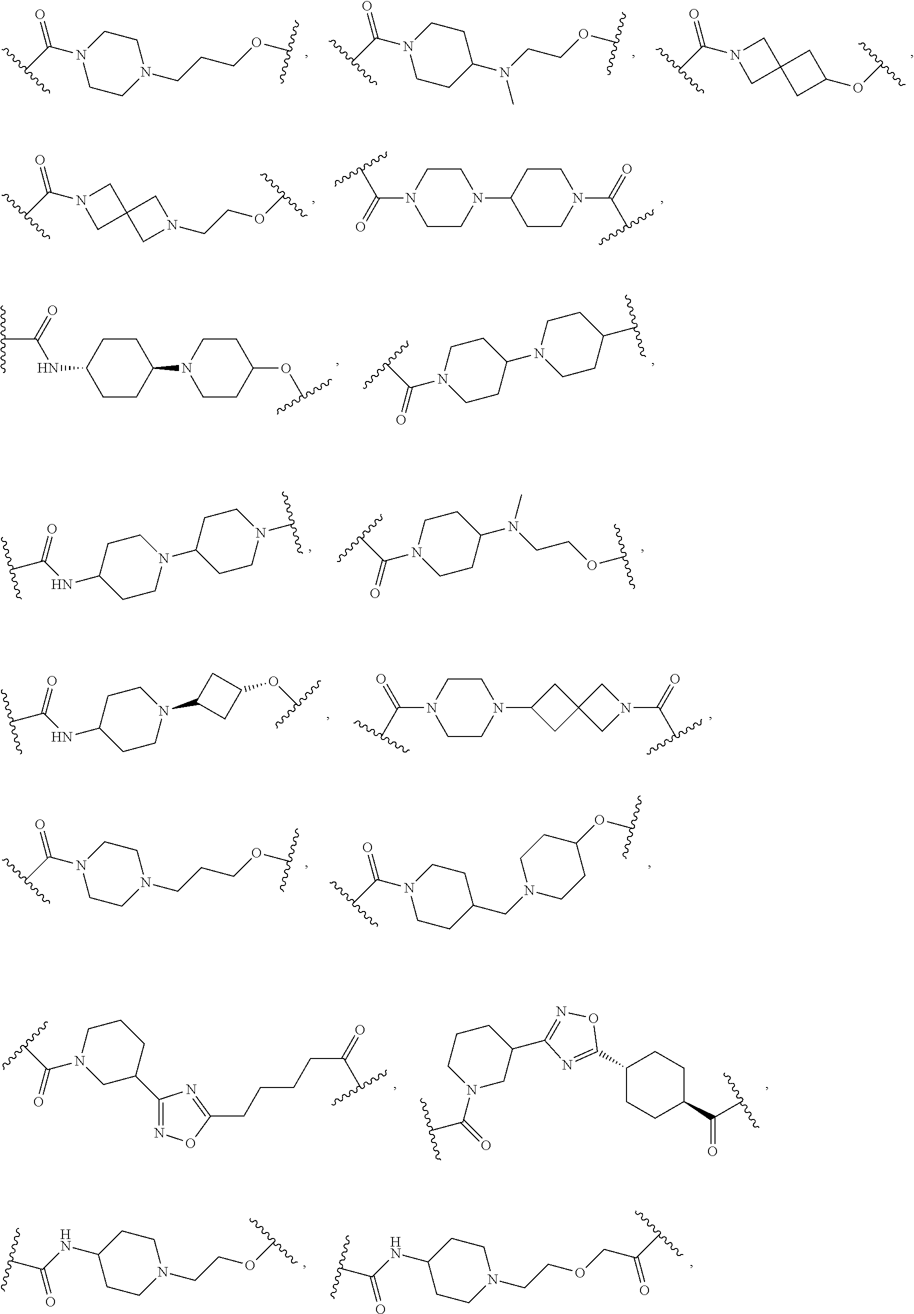

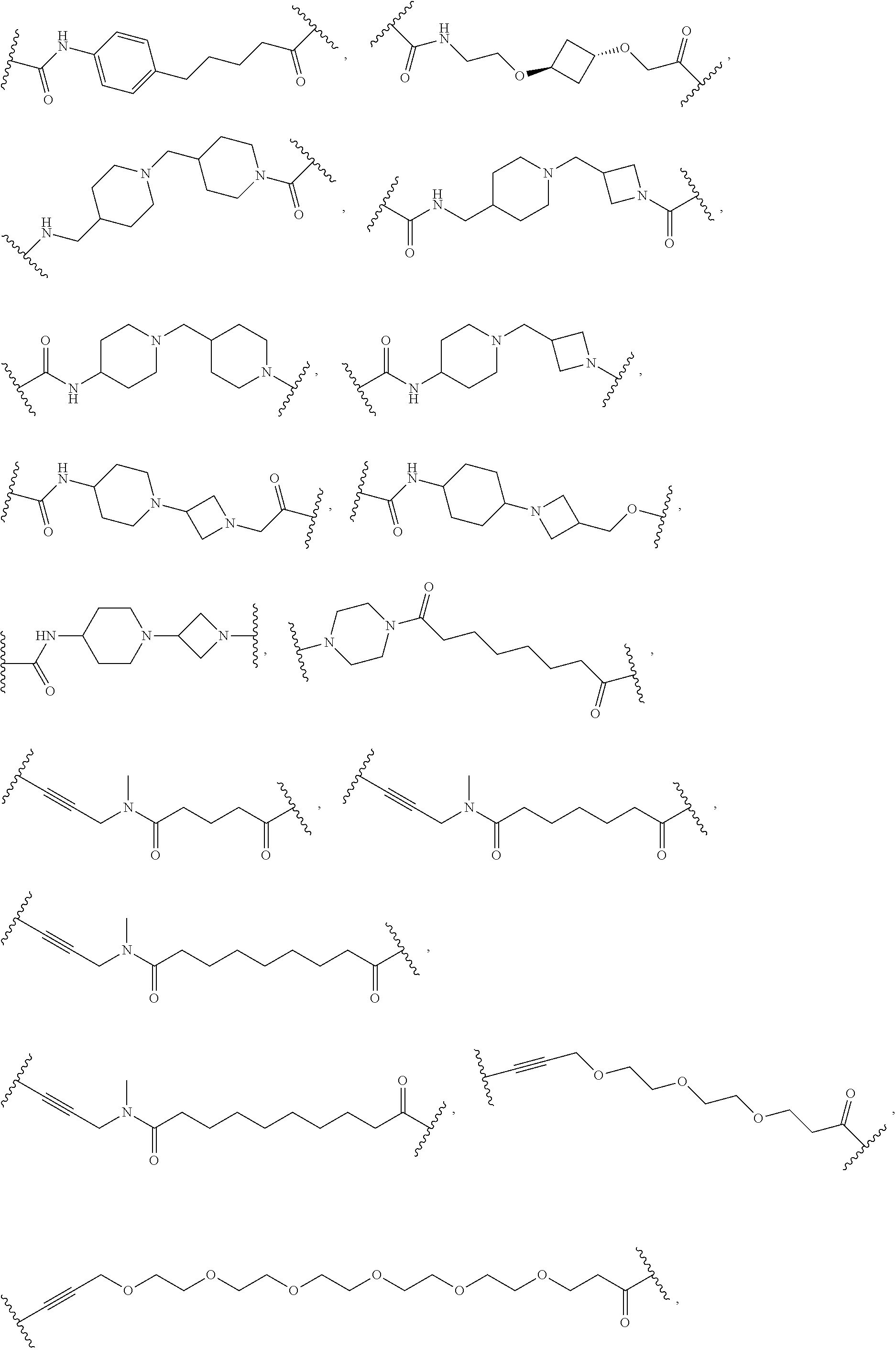

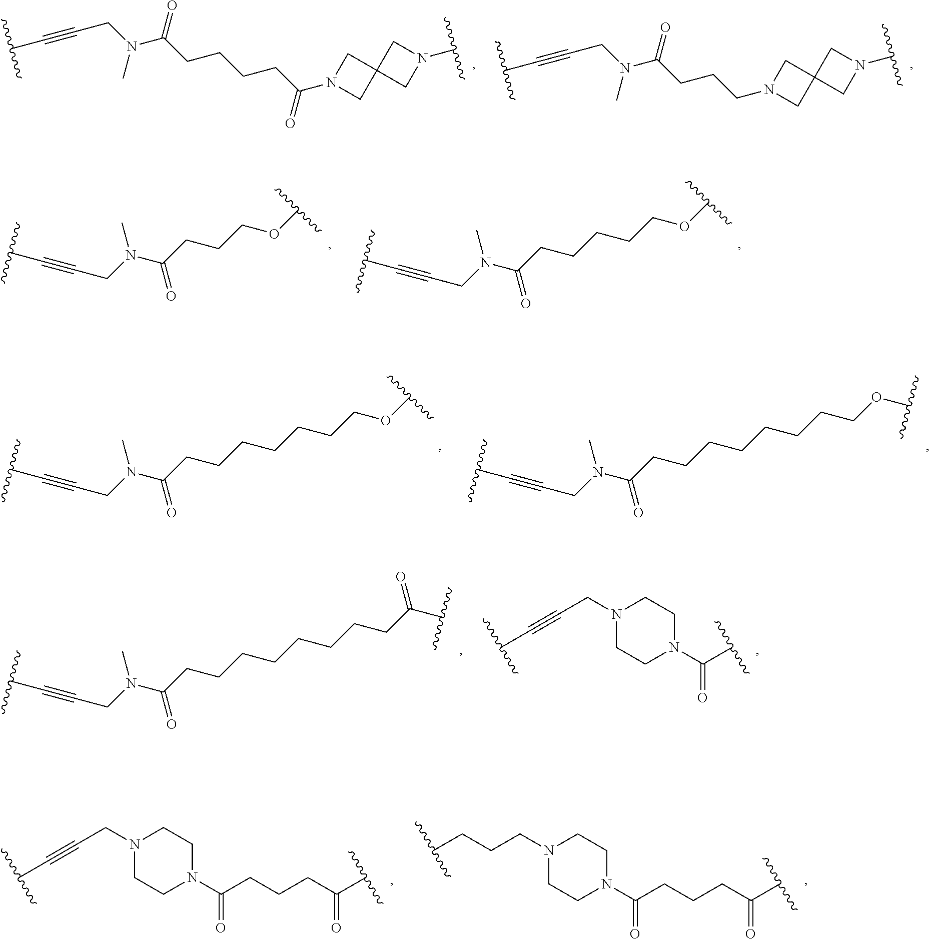

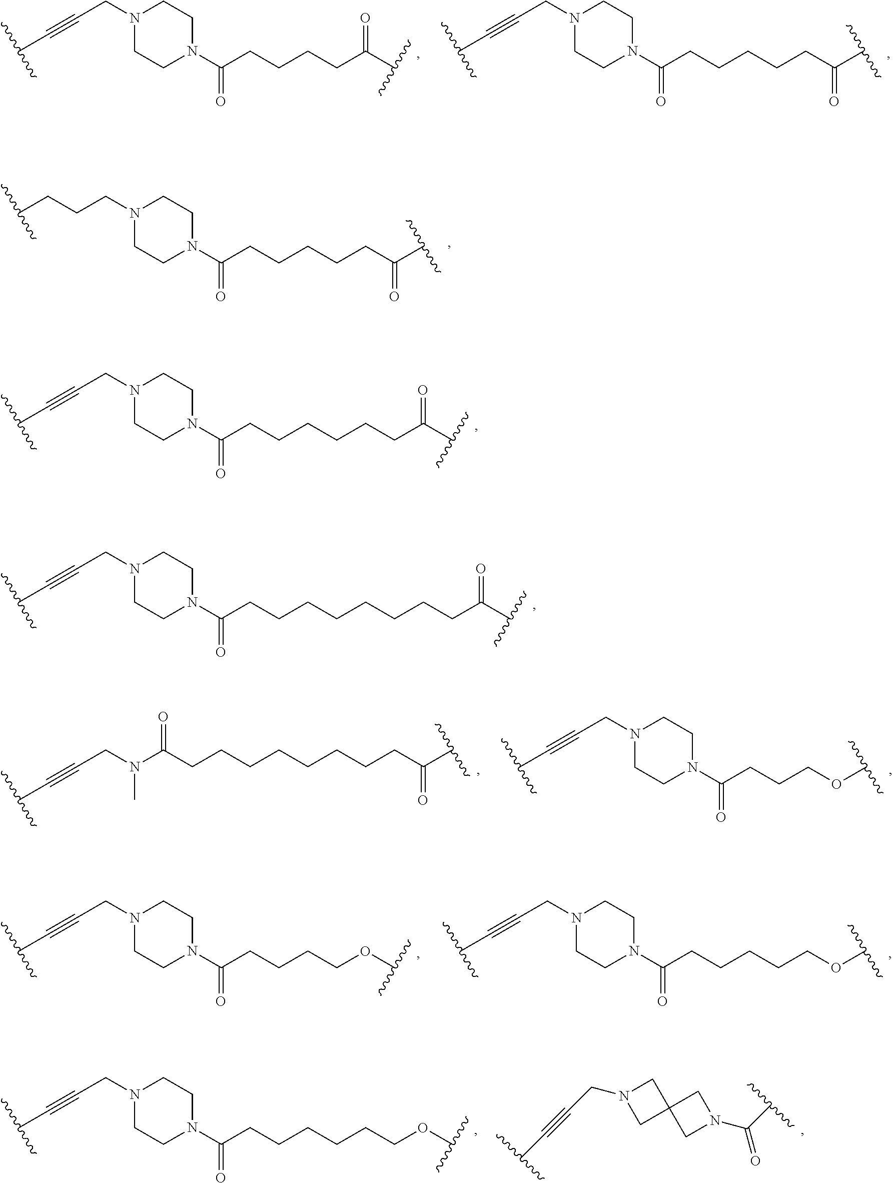

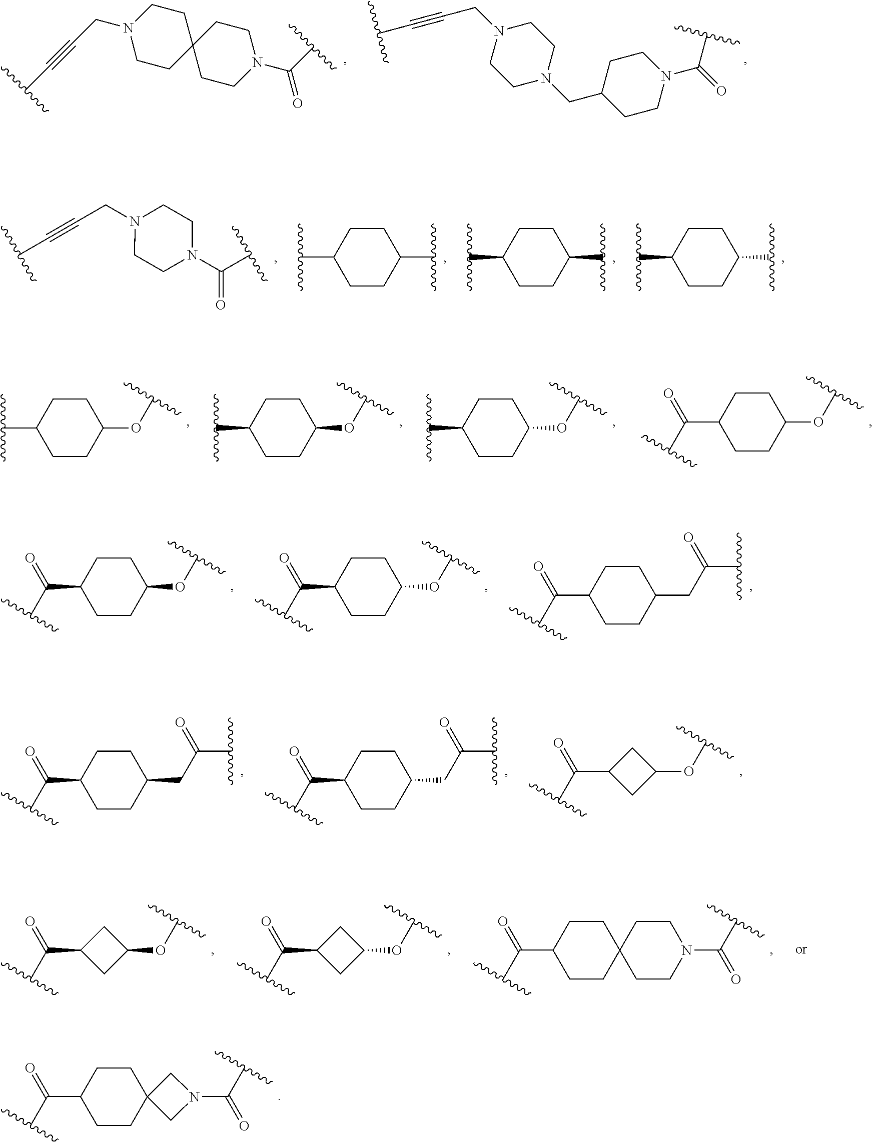

19. A compound selected from any one of the compounds depicted in Table 1, or a pharmaceutically acceptable salt thereof.

20. A pharmaceutical composition comprising a compound according to claim 1, and a pharmaceutically acceptable carrier, adjuvant, or vehicle.

21. The pharmaceutical composition according to claim 20, further comprising an additional therapeutic agent.

22. A method of degrading BCL-XL protein in a patient or biological sample comprising administering to said patient or contacting said biological sample with a compound according to claim 1, or a pharmaceutical composition thereof.

23. A method of treating an BCL-XL-mediated disorder, disease, or condition in a patient comprising administering to said patient a compound according to claim 1, or a pharmaceutical composition thereof.

24. The method of claim 23, further comprising administration of an additional therapeutic agent.

25. The method of claim 24, wherein the BCL-XL-mediated disorder, disease or condition is a cancer, an autoimmune disease, or inflammation.

26. The method of claim 25, wherein the cancer is selected from synovial sarcoma, Burkitt lymphoma, Hodgkin lymphoma, multiple myeloma, neuroblastoma, glioblastoma, small cell lung cancer, pancreatic cancer, hepatocellular (liver) cancer, endometrial cancer, ovarian cancer, cervical cancer, breast cancer, prostate cancer, bladder cancer, melanoma, rhabdomyosarcoma, osteosarcoma/malignant fibrous histiocytoma of bone, choriocarcinoma, kidney cancer (renal cell cancer), thyroid cancer, and leukemias (acute lymphoblastic, acute myeloid, chronic lymphocytic, and chronic myelogenous).
